(19) United States
(12) Patent Application Publication
(10) Pub. No.: US 2025/0099448 A1
(43) Pub. Date: Mar. 27, 2025

(54) HETEROCYCLIC COMPOUNDS

(71) Applicant: Hoffmann-La Roche Inc., Little Falls, NJ (US)

(72) Inventors: Machoud AMOUSSA, Berlin (DE); Joerg BENZ, Rheinfelden (DE); Julie Elisabeth Francoise BLAISING, Basel (CH); Jason Jacques DENIZOT, Savigny-en-Sancerre (FR); Kallie FRISTON, Stockton-On-Tees (GB); Rudolf Liun Zaccaria GANZONI, Zuerich (CH); Maude GIROUD, Basel (CH); Uwe GRETHER, Efringen-Kirchen (DE); Benoit HORNSPERGER, Altkirch (FR); Isabelle KAUFMANN, Wettingen (CH); Bernd KUHN, Reinach BL (CH); Camiel John LEAKE, Brasschaat (BE); Jacopo MARGARINI, Zuerich (CH); Rainer Eugen MARTIN, Basel (CH); Fionn Susannah O'HARA, Basel (CH); Bernd PUELLMANN, Aesch (CH); Martin RITTER, Mumpf (CH); Didier ROMBACH, Mulhouse (FR); Valerie RUNTZ-SCHMITT, Rixheim (FR); Philipp Claudio SCHMID, Birsfelden (CH); Matthias Beat WITTWER, Riehen (CH)

(73) Assignee: Hoffmann-La Roche Inc., Little Falls, NJ (US)

(21) Appl. No.: 18/832,840

(22) PCT Filed: Jan. 25, 2023
(86) PCT No.: PCT/EP2023/051723

§ 371 Date: Jul. 24, 2024

(30) Foreign Application Priority Data
Jan. 25, 2022 (EP) 22153215.3
Oct. 24, 2022 (EP) 22203186.6

(51) Int. Cl. A61K 31/4439; A61K 31/4196; A61K 31/497; A61K 31/501; A61K 31/506; A61K 31/675; C07D 401/14; C07D 403/14; C07D 487/10

(52) U.S. Cl. CPC A61K 31/4439; A61K 31/4196; A61K 31/497; A61K 31/501; A61K 31/506; A61K 31/675; C07D 401/14; C07D 403/14; C07D 487/10

(57) ABSTRACT

The invention provides new heterocyclic compounds having the general formula (I) wherein A and R¹ to R⁴ are as described heroin, compositions including the compounds, processes of manufacturing the compounds and methods of using the compounds.

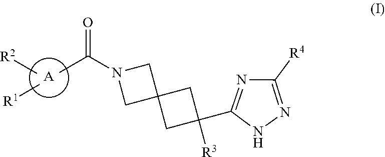

### FIELD OF THE INVENTION

The present invention relates to organic compounds useful for therapy or prophylaxis in a mammal, and in particular to monoacylglycerol lipase (MAGL) inhibitors for the treatment or prophylaxis of neuroinflammation, neurodegenerative diseases, pain, cancer, mental disorders, multiple sclerosis, Alzheimer's disease, Parkinson's disease, amyotrophic lateral sclerosis, traumatic brain injury, neurotoxicity, stroke, epilepsy, anxiety, migraine, depression, inflammatory bowel disease, abdominal pain, abdominal pain associated with irritable bowel syndrome and/or visceral pain in a mammal.

### BACKGROUND OF THE INVENTION

Endocannabinoids (ECs) are signaling lipids that exert their biological actions by interacting with cannabinoid receptors (CBRs), CB1 and CB2. They modulate multiple physiological processes including neuroinflammation, neurodegeneration and tissue regeneration (Iannotti, F. A., et al., Progress in lipid research 2016, 62, 107-28.). In the brain, the main endocannabinoid, 2-arachidonoylglycerol (2-AG), is produced by diacyglycerol lipases (DAGL) and hydrolyzed by the monoacylglycerol lipase, MAGL. MAGL hydrolyses 85% of 2-AG; the remaining 15% being hydrolysed by ABHD6 and ABDH12 (Nomura, D. K., et al., Science 2011, 334, 809.). MAGL is expressed throughout the brain and in most brain cell types, including neurons, astrocytes, oligodendrocytes and microglia cells (Chanda, P. K., et al., Molecular pharmacology 2010, 78, 996; Viader, A., et al., Cell reports 2015, 12, 798.). 2-AG hydrolysis results in the formation of arachidonic acid (AA), the precursor of prostaglandins (PGs) and leukotrienes (LTs). Oxidative metabolism of AA is increased in inflamed tissues. There are two principal enzyme pathways of arachidonic acid oxygenation involved in inflammatory processes, the cyclo-oxygenase which produces PGs and the 5-lipoxygenase which produces LTs. Of the various cyclooxygenase products formed during inflammation, PGE2 is one of the most important. These products have been detected at sites of inflammation, e.g. in the cerebrospinal fluid of patients suffering from neurodegenerative disorders and are believed to contribute to inflammatory response and disease progression. Mice lacking MAGL (Mgll−/−) exhibit dramatically reduced 2-AG hydrolase activity and elevated 2-AG levels in the nervous system while other arachidonoyl-containing phospho- and neutral lipid species including anandamide (AEA), as well as other free fatty acids, are unaltered. Conversely, levels of AA and AA-derived prostaglandins and other eicosanoids, including prostaglandin E2 (PGE2), D2 (PGD2), F2 (PGF2), and thromboxane B2 (TXB2), are strongly decreased. Phospholipase A₂ (PLA₂) enzymes have been viewed as the principal source of AA, but cPLA2-deficient mice have unaltered AA levels in their brain, reinforcing the key role of MAGL in the brain for AA production and regulation of the brain inflammatory process.

Neuroinflammation is a common pathological change characteristic of diseases of the brain including, but not restricted to, neurodegenerative diseases (e.g. multiple sclerosis, Alzheimer's disease, Parkinson disease, amyotrophic lateral sclerosis, traumatic brain injury, neurotoxicity, stroke, epilepsy and mental disorders such as anxiety and migraine). In the brain, production of eicosanoids and prostaglandins controls the neuroinflammation process. The pro-inflammatory agent lipopolysaccharide (LPS) produces a robust, time-dependent increase in brain eicosanoids that is markedly blunted in Mgll−/− mice. LPS treatment also induces a widespread elevation in pro-inflammatory cytokines including interleukin-1-a (IL-1-a), IL-1b, IL-6, and tumor necrosis factor-a (TNF-α) that is prevented in Mgll−/− mice.

Neuroinflammation is characterized by the activation of the innate immune cells of the central nervous system, the microglia and the astrocytes. It has been reported that anti-inflammatory drugs can suppress in preclinical models the activation of glia cells and the progression of disease including Alzheimer's disease and multiple sclerosis (Lleo A., Cell Mol Life Sci. 2007, 64, 1403.). Importantly, genetic and/or pharmacological disruption of MAGL activity also blocks LPS-induced activation of microglial cells in the brain (Nomura, D. K., et al., Science 2011, 334, 809.).

In addition, genetic and/or pharmacological disruption of MAGL activity was shown to be protective in several animal models of neurodegeneration including, but not restricted to, Alzheimer's disease, Parkinson's disease and multiple sclerosis. For example, an irreversible MAGL inhibitor has been widely used in preclinical models of neuroinflammation and neurodegeneration (Long, J. Z., et al., Nature chemical biology 2009, 5, 37.). Systemic injection of such inhibitor recapitulates the Mgll−/− mice phenotype in the brain, including an increase in 2-AG levels, a reduction in AA levels and related eicosanoids production, as well as the prevention of cytokines production and microglia activation following LPS-induced neuroinflammation (Nomura, D. K., et al., Science 2011, 334, 809.), altogether confirming that MAGL is a druggable target.

Consecutive to the genetic and/or pharmacological disruption of MAGL activity, the endogenous levels of the MAGL natural substrate in the brain, 2-AG, are increased. 2-AG has been reported to show beneficial effects on pain with, for example, anti-nociceptive effects in mice (Ignatowska-Jankowska B. et al., J. Pharmacol. Exp. Ther. 2015, 353, 424.) and on mental disorders, such as depression in chronic stress models (Zhong P. et al., Neuropsychopharmacology 2014, 39, 1763.).

Furthermore, oligodendrocytes (OLs), the myelinating cells of the central nervous system, and their precursors (OPCs) express the cannabinoid receptor 2 (CB2) on their membrane. 2-AG is the endogenous ligand of CB1 and CB2 receptors. It has been reported that both cannabinoids and pharmacological inhibition of MAGL attenuate OLs's and OPCs's vulnerability to excitotoxic insults and therefore may be neuroprotective (Bernal-Chico, A., et al., Glia 2015, 63, 163.). Additionally, pharmacological inhibition of MAGL increases the number of myelinating OLs in the brain of mice, suggesting that MAGL inhibition may promote differentiation of OPCs in myelinating OLs in vivo (Alpar, A., et al., Nature communications 2014, 5, 4421.). Inhibition of MAGL was also shown to promote remyelination and functional recovery in a mouse model of progressive multiple sclerosis (Feliu A. et al., Journal of Neuroscience 2017, 37 (35), 8385.).

In addition, in recent years, metabolism is talked highly important in cancer research, especially the lipid metabolism. Researchers believe that the de novo fatty acid synthesis plays an important role in tumor development. Many studies illustrated that endocannabinoids have anti-tumorigenic actions, including anti-proliferation, apoptosis induction and anti-metastatic effects. MAGL as an important decomposing enzyme for both lipid metabolism and the endocannabinoids system, additionally as a part of a gene expression signature, contributes to different aspects of tumourigenesis, including in glioblastoma (Qin, H., et al., Cell Biochem. Biophys. 2014, 70, 33; Nomura D K et al., Cell 2009, 140(1), 49-61; Nomura D K et al., Chem. Biol. 2011, 18(7), 846-856, Jinlong Yin et al, Nature Communications 2020, 11, 2978).

The endocannabinoid system is also involved in many gastrointestinal physiological and physiopathological actions (Marquez, Suarez et al. 2009). All these effects are driven mainly via cannabinoid receptors (CBRs), CB1 and CB2. CB1 receptors are present throughout the GI tract of animals and healthy humans, especially in the enteric nervous system (ENS) and the epithelial lining, as well as smooth muscle cells of blood vessels in the colonic wall (Wright, Rooney et al. 2005), (Duncan, Davison et al. 2005). Activation of CB1 produces anti-emetic, anti-motility, and anti-inflammatory effect, and help to modulate pain (Perisetti, Rimu et al. 2020). CB2 receptors are expressed in immune cells such as plasma cells and macrophages, in the lamina propria of the GI tract (Wright, Rooney et al. 2005), and primarily on the epithelium of human colonic tissue associated with inflammatory bowel disease (IBD). Activation of CB2 exerts anti-inflammatory effect by reducing pro-inflammatory cytokines. Expression of MAGL is increased in colonic tissue in UC patients (Marquez, Suarez et al. 2009) and 2-AG levels are increased in plasma of IBD patients (Grill, Hogenauer et al. 2019). Several animal studies have demonstrated the potential of MAGL inhibitors for symptomatic treatment of IBD. MAGL inhibition prevents TNBS-induced mouse colitis and decreases local and circulating inflammatory markers via a CB1/CB2 MoA (Marquez, Suarez et al. 2009). Furthermore, MAGL inhibition improves gut wall integrity and intestinal permeability via a CB1 driven MoA (Wang, Zhang et al. 2020).

In conclusion, suppressing the action and/or the activation of MAGL is a promising new therapeutic strategy for the treatment or prevention of neuroinflammation, neurodegenerative diseases, pain, cancer, mental disorders, inflammatory bowel disease, abdominal pain and abdominal pain associated with irritable bowel syndrome. Furthermore, suppressing the action and/or the activation of MAGL is a promising new therapeutic strategy for providing neuroprotection and myelin regeneration. Accordingly, there is a high unmet medical need for new MAGL inhibitors.

### SUMMARY OF THE INVENTION

In a first aspect, the present invention provides compounds of formula (I)

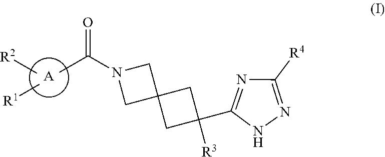

wherein A and R¹ to R⁴ are as defined herein.

In a further aspect, the present invention provides a process of manufacturing the compounds of formula (I) described herein, or pharmaceutically acceptable salts thereof, wherein the process is as described in any one of schemes 1 to 40.

In a further aspect, the present invention provides a compound of formula (I) as described herein, or a pharmaceutically acceptable salt thereof, when manufactured according to the processes described herein.

In a further aspect, the present invention provides a compound of formula (I) as described herein, or a pharmaceutically acceptable salt thereof, for use as therapeutically active substance.

In a further aspect, the present invention provides a pharmaceutical composition comprising a compound of formula (I) as described herein, or a pharmaceutically acceptable salt thereof, and a therapeutically inert carrier.

In a further aspect, the present invention provides a compound of formula (I) as described herein, or a pharmaceutically acceptable salt thereof, for use in a method of inhibiting monoacylglycerol lipase in a mammal.

In a further aspect, the present invention provides a compound of formula (I) as described herein, or a pharmaceutically acceptable salt thereof, for use in the treatment or prophylaxis of neuroinflammation, neurodegenerative diseases, pain, cancer, mental disorders and/or inflammatory bowel disease in a mammal.

In a further aspect, the present invention provides a compound of formula (I) as described herein, or a pharmaceutically acceptable salt thereof, for use in the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “alkyl” refers to a mono- or multivalent, e.g., a mono- or bivalent, linear or branched saturated hydrocarbon group of 1 to 12 carbon atoms. In some preferred embodiments, the alkyl group contains 1 to 6 carbon atoms (“C₁₋₆-alkyl”), e.g., 1, 2, 3, 4, 5, or 6 carbon atoms. In other embodiments, the alkyl group contains 1 to 3 carbon atoms, e.g., 1, 2 or 3 carbon atoms. Some non-limiting examples of alkyl include methyl, ethyl, propyl, 2-propyl (isopropyl), n-butyl, iso-butyl, sec-butyl, tert-butyl, and 2,2-dimethylpropyl. Particularly preferred, yet non-limiting examples of alkyl are methyl, ten-butyl, and 2,2-dimethylpropyl.

The term “alkoxy” refers to an alkyl group, as previously defined, attached to the parent molecular moiety via an oxygen atom. Unless otherwise specified, the alkoxy group contains 1 to 12 carbon atoms. In some preferred embodiments, the alkoxy group contains 1 to 6 carbon atoms (“C₁₋₆-alkoxy”). In other embodiments, the alkoxy group contains 1 to 4 carbon atoms. In still other embodiments, the alkoxy group contains 1 to 3 carbon atoms. Some non-limiting examples of alkoxy groups include methoxy, ethoxy, n-propoxy, isopropoxy, n-butoxy, isobutoxy and tert-butoxy. A particularly preferred, yet non-limiting example of alkoxy is methoxy.

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

The term “cycloalkyl” as used herein refers to a saturated or partly unsaturated monocyclic or bicyclic hydrocarbon group of 3 to 10 ring carbon atoms (“C₃₋₁₀-cycloalkyl”). In some preferred embodiments, the cycloalkyl group is a saturated monocyclic hydrocarbon group of 3 to 8 ring carbon atoms. “Bicyclic cycloalkyl” refers to cycloalkyl moieties consisting of two saturated carbocycles having two carbon atoms in common, i.e., the bridge separating the two rings is either a single bond or a chain of one or two ring atoms, and to spirocyclic moieties, i.e., the two rings are connected via one common ring atom. Preferably, the cycloalkyl group is a saturated monocyclic hydrocarbon group of 3 to 6 ring carbon atoms, e.g., of 3, 4, 5 or 6 carbon atoms. Some non-limiting examples of cycloalkyl include cyclopropyl, cyclobutyl, cyclopentyl, cyclohexyl, cycloheptyl, 1-bicyclo[1.1.1]pentanyl, norbornanyl, and 1-bicyclo[2.2.2]octanyl. A particularly preferred, yet non-limiting example of cycloalkyl is cyclopropyl.

The term “aryl” refers to a monocyclic, bicyclic, or tricyclic carbocyclic ring system having a total of 6 to 14 ring members (“C₆-C₁₄-aryl”), preferably 6 to 12 ring members, and more preferably 6 to 10 ring members, and wherein at least one ring in the system is aromatic. Some non-limiting examples of aryl include phenyl and 9H-fluorenyl (e.g. 9H-fluoren-9-yl). A particularly preferred, yet non-limiting example of aryl is phenyl.

The term “heteroaryl” refers to a mono- or multivalent, monocyclic, bicyclic or tricyclic, preferably monocyclic or bicyclic ring system having a total of 5 to 14 ring members, preferably, 5 to 12 ring members, more preferably 5 to 10 ring members, in particular 5 to 9 ring members, 5 to 8 ring members or 5 to 6 ring members, wherein at least one ring in the system is aromatic, and at least one ring in the system contains one or more heteroatoms. Preferably, “heteroaryl” refers to a 5 to 10 membered heteroaryl comprising 1, 2, 3 or 4 heteroatoms independently selected from O, S and N. Most preferably, “heteroaryl” refers to a 5-10 membered heteroaryl, 5-9 membered heteroaryl, 5-8 membered heteroaryl, or 5-6 membered heteroaryl comprising 1 to 2 heteroatoms independently selected from O, S and N. Some non-limiting examples of heteroaryl include spiro[cyclopropane-1,3′-indoline] (e.g., spiro[cyclopropane-1,3′-indoline]-1′-yl), 2-pyridyl, 3-pyridyl, 4-pyridyl, pyrazin-2-yl, pyrimidin-2-yl, pyrimidin-4-yl, pyrimidin-5-yl, pyrimidin-6-yl, indol-1-yl, 1H-indol-2-yl, 1H-indol-3-yl, 1H-indol-4-yl, 1H-indol-5-yl, 1H-indol-6-yl, 1H-indol-7-yl, 1,2-benzoxazol-3-yl, 1,2-benzoxazol-4-yl, 1,2-benzoxazol-5-yl, 1,2-benzoxazol-6-yl, 1,2-benzoxazol-7-yl, 1H-indazol-3-yl, 1H-indazol-4-yl, 1H-indazol-5-yl, 1H-indazol-6-yl, 1H-indazol-7-yl, pyrazol-1-yl, 1H-pyrazol-3-yl, 1H-pyrazol-4-yl, 1H-pyrazol-5-yl, pyrazolo[1,5-a]pyridine, 2H-pyrazolo[4,3-b]pyridine, [1,2,4]triazolo[1,5-a]pyridine, 1H-pyrrolo[2,3-b]pyridine, imidazol-1-yl, 1H-imidazol-2-yl, 1H-imidazol-4-yl, 1H-imidazol-5-yl, oxazol-2-yl, oxazol-4-yl, oxazol-5-yl, 1,2,4-oxadiazolyl, 1,3,4-oxadiazolyl, thiazol-2-yl, thiazol-4-yl, thiazol-5-yl, pyridazin-3-yl, pyridazin-4-yl, 1,2,4-triazol-4-yl, 1,2,4-triazol-1-yl, 4H-1,2,4-triazol-3-yl, trizaol-2-yl, 2H-triazolyl, 4,5,6,7-tetrahydroindazol-2-yl, 6,7-dihydro-4H-pyrano[4,3-c]pyrazol-2-yl, thiazolyl, benzofurazan-4-yl, tetrazolyl, isoxazolyl, pyrrolyl, and morpholinyl. Particularly preferred, yet non-limiting examples of heteroaryl are pyridyl, pyrazinyl, pyrimidinyl, pyrazolyl, imidazolyl, oxazolyl, oxadiazolyl and triazolyl.

The term “heterocyclyl” refers to a saturated or partly unsaturated mono- or bicyclic, preferably monocyclic ring system of 3 to 14 ring atoms, preferably 3 to 10 ring atoms, more preferably 3 to 8 ring atoms, more preferably 3 to 6 ring atoms, in particular 3, 4, 5 or 6 ring atoms, wherein 1, 2, or 3 of said ring atoms are heteroatoms selected from N, O and S, the remaining ring atoms being carbon. Preferably, 1 to 2 of said ring atoms are selected from N and O, the remaining ring atoms being carbon. “Bicyclic heterocyclyl” refers to heterocyclic moieties consisting of two cycles having two ring atoms in common, i.e., the bridge separating the two rings is either a single bond or a chain of one or two ring atoms, and to spirocyclic moieties, i.e., the two rings are connected via one common ring atom. Some non-limiting examples of heterocyclyl groups include azetidinyl, pyrrolidinyl, oxetanyl, 5-azaspiro[2.5]octan-5-yl, piperidyl, 3,3a,4,5,6,6a-hexahydro-1H-cyclopenta[c]pyrrol-2-yl, 2-azaspiro[3.3]heptan-2-yl, 2,6-diazaspiro[3.3]heptanyl, 2-azaspiro[3.4]octane, 2-azaspiro[3.5]nonan-2-yl, 1,2-dihydropyridiynl, piperidyl, and thietanyl.

The term “hydroxy” refers to an —OH group.

The term “cyano” refers to a —CN (nitrile) group.

The term “carboxy” refers to a —COOH group (i.e., a carboxylic acid group).

The term “alkoxycarbonyl” refers to a —C(O)—O—C₁-C₆-alkyl group (i.e., a carboxylic acid ester group).

The term “oxo” refers to a double bonded oxygen (═O).

The term “carbamoyl” refers to a group H₂N—C(O)—.

The term “haloalkyl” refers to an alkyl group, wherein at least one of the hydrogen atoms of the alkyl group has been replaced by a halogen atom, preferably fluoro. Preferably, “haloalkyl” refers to an alkyl group wherein 1, 2 or 3 hydrogen atoms of the alkyl group have been replaced by a halogen atom, most preferably fluoro. Particularly preferred, yet non-limiting examples of haloalkyl are trifluoromethyl, difluoromethyl, 1,1-difluoroethyl, 2,2-difluoroethyl, and 2,2,2-trifluoroethyl.

The term “hydroxyalkyl” refers to an alkyl group, wherein at least one of the hydrogen atoms of the alkyl group has been replaced by a hydroxy group. Preferably, “haloalkyl” refers to an alkyl group wherein 1, 2 or 3 hydrogen atoms, in particular 1 hydrogen atom, of the alkyl group have been replaced by a hydroxy group. A particularly preferred, yet non-limiting example of hydroxyalkyl is hydroxymethyl.

The term “carboxyalkyl” refers to an alkyl group, wherein at least one of the hydrogen atoms of the alkyl group has been replaced by carboxy group. Preferably, “carboxyalkyl” refers to an alkyl group wherein 1, 2 or 3 hydrogen atoms, in particular 1 hydrogen atom, of the alkyl group have been replaced by a carboxy group. A particularly preferred, yet non-limiting example of carboxyalkyl is 1-carboxy-1-methyl-ethyl.

The term “haloalkoxy” refers to an alkoxy group, wherein at least one of the hydrogen atoms of the alkoxy group has been replaced by a halogen atom, preferably fluoro. Preferably, “haloalkoxy” refers to an alkoxy group wherein 1, 2 or 3 hydrogen atoms of the alkoxy group have been replaced by a halogen atom, most preferably fluoro. Particularly preferred, yet non-limiting examples of haloalkoxy are trifluoromethoxy, difluoromethoxy, 2,2,2-trifluoro-1,1-dimethyl-ethoxy, (1,1,1-trifluoropropan-2-yl)oxy, and 2,2,2-trifluoroethoxy.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like.

The compounds of formula (I) can contain several asymmetric centers and can be present in the form of optically pure enantiomers, mixtures of enantiomers such as, for example, racemates, optically pure diastereioisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The abbreviation “MAGL” refers to the enzyme monoacylglycerol lipase. The terms “MAGL” and “monoacylglycerol lipase” are used herein interchangeably.

The term “treatment” as used herein includes: (1) inhibiting the state, disorder or condition (e.g. arresting, reducing or delaying the development of the disease, or a relapse thereof in case of maintenance treatment, of at least one clinical or subclinical symptom thereof); and/or (2) relieving the condition (i.e., causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

The term “prophylaxis” as used herein includes: preventing or delaying the appearance of clinical symptoms of the state, disorder or condition developing in a mammal and especially a human that may be afflicted with or predisposed to the state, disorder or condition but does not yet experience or display clinical or subclinical symptoms of the state, disorder or condition.

The term “neuroinflammation” as used herein relates to acute and chronic inflammation of the nervous tissue, which is the main tissue component of the two parts of the nervous system; the brain and spinal cord of the central nervous system (CNS), and the branching peripheral nerves of the peripheral nervous system (PNS). Chronic neuroinflammation is associated with neurodegenerative diseases such as Alzheimer's disease, Parkinson's disease and multiple sclerosis. Acute neuroinflammation usually follows injury to the central nervous system immediately, e.g., as a result of traumatic brain injury (TBI).

The term “traumatic brain injury” (“TB”, also known as “intracranial injury”), relates to damage to the brain resulting from external mechanical force, such as rapid acceleration or deceleration, impact, blast waves, or penetration by a projectile.

The teen “neurodegenerative diseases” relates to diseases that are related to the progressive loss of structure or function of neurons, including death of neurons. Examples of neurodegenerative diseases include, but are not limited to, multiple sclerosis, Alzheimer's disease, Parkinson's disease and amyotrophic lateral sclerosis.

The term “mental disorders” (also called mental illnesses or psychiatric disorders) relates to behavioral or mental patterns that may cause suffering or a poor ability to function in life. Such features may be persistent, relapsing and remitting, or occur as a single episode. Examples of mental disorders include, but are not limited to, anxiety and depression.

The term “pain” relates to an unpleasant sensory and emotional experience associated with actual or potential tissue damage. Examples of pain include, but are not limited to, nociceptive pain, chronic pain (including idiopathic pain), neuropathic pain including chemotherapy induced neuropathy, phantom pain and phsychogenic pain. A particular example of pain is neuropathic pain, which is caused by damage or disease affecting any part of the nervous system involved in bodily feelings (i.e., the somatosensory system). In one embodiment, “pain” is neuropathic pain resulting from amputation or thoracotomy. In one embodiment, “pain” is chemotherapy induced neuropathy.

The term “neurotoxicity” relates to toxicity in the nervous system. It occurs when exposure to natural or artificial toxic substances (neurotoxins) alter the normal activity of the nervous system in such a way as to cause damage to nervous tissue. Examples of neurotoxicity include, but are not limited to, neurotoxicity resulting from exposure to substances used in chemotherapy, radiation treatment, drug therapies, drug abuse, and organ transplants, as well as exposure to heavy metals, certain foods and food additives, pesticides, industrial and/or cleaning solvents, cosmetics, and some naturally occurring substances.

The term “cancer” refers to a disease characterized by the presence of a neoplasm or tumor resulting from abnormal uncontrolled growth of cells (such cells being “cancer cells”). As used herein, the term cancer explicitly includes, but is not limited to, hepatocellular carcinoma, colon carcinogenesis and ovarian cancer.

The term “mammal” as used herein includes both humans and non-humans and includes but is not limited to humans, non-human primates, canines, felines, murines, bovines, equines, and porcines. In a particularly preferred embodiment, the term “mammal” refers to humans.

### COMPOUNDS OF THE INVENTION

In a first aspect, the present invention provides a compound of Formula (I)

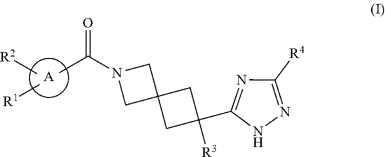

or a pharmaceutically acceptable salt thereof, wherein:
A is selected from:

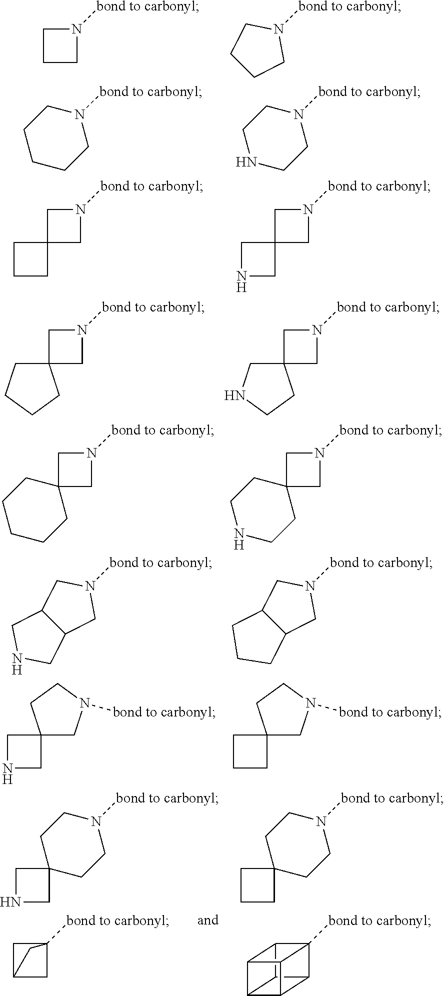

B is selected from C₆-C₁₄-aryl, C₃-C₁₀-cycloalkyl, 5- to 14-membered heteroaryl, and 3- to 14-membered heterocyclyl;
C is selected from C₆-C₁₄-aryl, C₃-C₁₀-cycloalkyl, 5- to 14-membered heteroaryl, heterocyclyl, and 5- to 14-membered heteroaryl;
L¹ is selected from a covalent bond, carbonyl, —CR^{8a}R^{8b}—, —CH₂O—, —OCH₂—, —CH₂NR^{13a}—, —NR^{13b}CH₂—, —CH₂CH₂—, —CH₂NR^{13c}CH₂—, —CH₂NHCO—, —O—, —NH—, —SO₂NH—, —NHSO₂—, —SO₂NHCH₂—, —CH₂NHSO₂—, —SO₂—, —CH₂SO₂—, —SO₂CH₂—, —NHC(O)—, —C(O)NH—, and

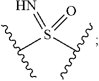 [SMILES: *S(*)(=N)=O]

L² is selected from a covalent bond, —CR^{8a}R^{8b}—, —NH—, —N(C₁-C₆-alkyl)-, —O—, —CH₂NH—, —NHCH₂—, —CH₂O—, —OCH₂—, —SO₂—, —CH₂SO₂—, and —SO₂CH₂—;
X is NH or O;
R¹ is selected from a group

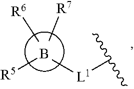

C₁-C₆-alkyl-SO₂—NH—, C₁-C₆-alkyl-SO₂—, and halo-C₁-C₆-alkyl-C(O)—;
R² is selected from hydrogen and hydroxy;
R³ is selected from hydrogen, halogen, cyano, C₁-C₆-alkyl, C₁-C₆-alkoxy, and hydroxy;
R⁴ is selected from hydrogen and a group —C(R^{4a}R^{4b}R^{4c});
R^{4a} is selected from C₁-C₆-alkyl and halo-C₁-C₆-alkyl; R^{4b} is selected from hydrogen and hydroxy; and R^{4c} is hydrogen; or
R^{4a}, R^{4b}, and R^{4c} are each independently selected from hydrogen and halogen; or
R^{4a} and R^{4b}, taken together with the carbon atom to which they are attached, form a C₃-C₁₀-cycloalkyl or a 3- to 14-membered heterocyclyl; and R^{4c} is selected from hydrogen, hydroxy, amino, and halogen; wherein said C₃-C₁₀-cycloalkyl and 3- to 14-membered heterocyclyl are optionally substituted with 1-2 halogen substituents;
R⁵ is selected from hydrogen, hydroxy, halogen, cyano, C₁-C₆-alkyl, C₁-C₆-alkoxy, halo-C₁-C₆-alkyl, hydroxy-C₁-C₆-alkyl, hydroxy-halo-C₁-C₆-alkyl, carboxy-C₁-C₆-alkyl, halo-C₁-C₆-alkoxy, oxo, NH₂—SO₂—, amino, C₁-C₆-alkyl-NH—, (C₁-C₆-alkyl)₂N—, carbamoyl, C₁-C₆-alkyl-NH—C(O)—, (C₁-C₆-alkyl)₂N—C(O)—, carboxy, C₁-C₆-alkoxycarbonyl, C₁-C₆-alkoxycarbonyl-C₁-C₆-alkyl-, (C₁-C₆-alkyl)₂P(O)—, (C₁-C₆-alkyl)₂P(O)—C₁-C₆-alkyl-, a group

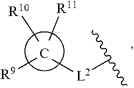

and a group

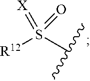

R⁶ is selected from hydrogen, halogen, cyano, oxo, C₁-C₆-alkoxy, halo-C₁-C₆-alkoxy, halo-C₁-C₆-alkyl, and C₁-C₆-alkyl-SO₂—;
R⁷ is selected from hydrogen and C₁-C₆-alkyl;
R^{8a} is selected from hydrogen, halogen, C₁-C₆-alkyl, and carbamoyl;
R^{8b} is selected from hydrogen and halogen;
R⁹ is selected from hydrogen, halogen, cyano, hydroxy, oxo, carboxy, C₁-C₆-alkoxycarbonyl, amino, C₁-C₆-alkyl-NH—, (C₁-C₆-alkyl)₂N—, carbamoyl, C₁-C₆-alkyl-NH—C(O)—, C₁-C₆-alkyl, hydroxy-C₁-C₆-alkyl, halo-C₁-C₆-alkyl, C₁-C₆-alkoxy-C₁-C₆-alkyl, C₁-C₆-alkoxy, halo-C₁-C₆-alkoxy, C₃-C₁₀-cycloalkyl, cyano, C₁-C₆-alkyl-SO₂—, halo-C₁-C₆-alkyl-SO₂—, and 5- to 14-membered heteroaryl; wherein said C₃-C₁₀-cycloalkyl and 5- to 14-membered heteroaryl is optionally substituted with one substituent selected from C₁-C₆-alkyl and halo-C₁-C₆-alkyl; or
R⁹ and R⁵ together form a group

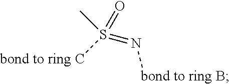

R¹⁰ is selected from hydrogen, halogen, C₁-C₆-alkyl, halo-C₁-C₆-alkyl, and oxo;
R¹¹ is selected from hydrogen and halogen;
R¹² is selected from amino, C₁-C₆-alkyl and halo-C₁-C₆-alkyl; and
R^{13a}, R^{13b}, and R^{13c} are each independently selected from hydrogen, C₁-C₆-alkyl, C₃-C₁₀-cycloalkyl, C₃-C₁₀-cycloalkyl-C₁-C₆-alkyl-.

In one embodiment, the present invention provides a compound of formula (I) as described herein, or a pharmaceutically acceptable salt thereof, wherein:
A is selected from:

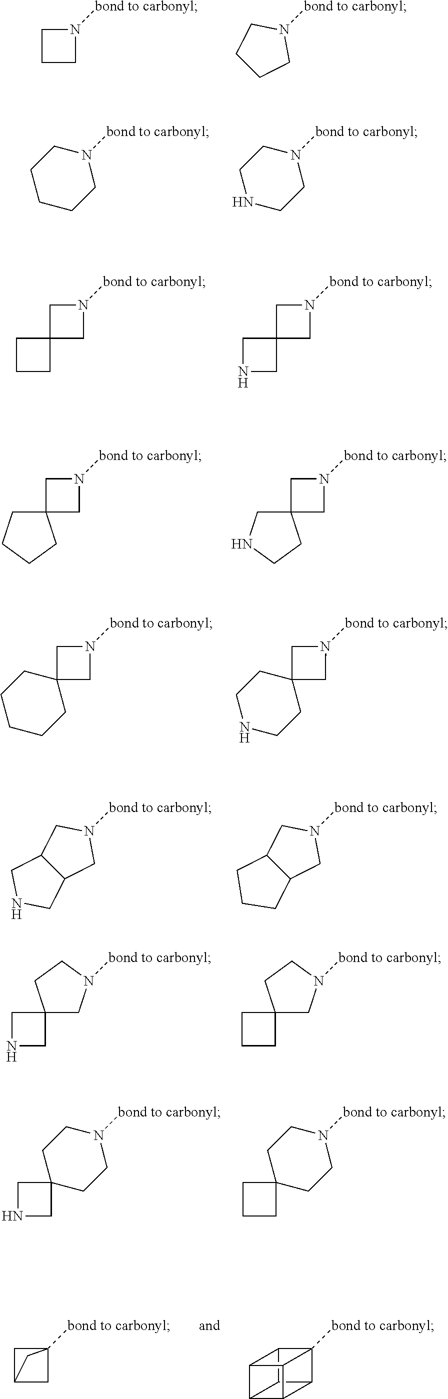

B is selected from C₆-C₁₄-aryl, C₃-C₁₀-cycloalkyl, 5- to 14-membered heteroaryl, and 3- to 14-membered heterocyclyl;
C is selected from C₆-C₁₄-aryl, C₃-C₁₀-cycloalkyl, 3- to 14-membered heterocyclyl, and 5- to 14-membered heteroaryl;
L¹ is selected from a covalent bond, carbonyl, —CR^{8a}R^{8b}—, —CH₂O—, —OCH₂—, —CH₂NR^{13a}, —NR^{13b}CH₂—, —CH₂CH₂—, —CH₂NR^{13c}CH₂, —CH₂NHCO—, —O—, —NH—, —SO₂NH—, —NHSO₂—, —SO₂NHCH₂—, —CH₂NHSO₂—, —SO₂—, —CH₂SO₂-, —SO₂CH₂—, —NHC(O)—, —C(O)NH—, and

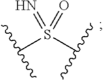 [SMILES: *S(*)(=N)=O]

L² is selected from a covalent bond, —CR^{8a}R^{8b}—, —NH—, —N(C₁-C₆-alkyl)-, —O—, —CH₂NH—, —NHCH₂—, —CH₂O—, —OCH₂—, —SO₂—, —CH₂SO₂—, and —SO₂CH₂—;
X is NH or O;
R¹ is selected from a group

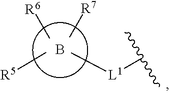

C₁-C₆-alkyl-SO₂—NH—, C₁-C₆-alkyl-SO₂—, and halo-C₁-C₆-alkyl-C(O)—;
R² is selected from hydrogen and hydroxy;
R³ is selected from hydrogen, halogen, cyano, C₁-C₆-alkyl, C₁-C₆-alkoxy, and hydroxy;
R⁴ is selected from hydrogen and a group —C(R^{4a}R^{4b}R^{4c});
R^{4a} is selected from C₁-C₆-alkyl and halo-C₁-C₆-alkyl; R^{4b} is selected from hydrogen and hydroxy; and R^{4c} is hydrogen; or
R^{4a}, R^{4b}, and R^{4c} are each independently selected from hydrogen and halogen; or
R^{4a} and R^{4b}, taken together with the carbon atom to which they are attached, form a C₃-C₁₀-cycloalkyl or a 3- to 14-membered heterocyclyl; and R^{4c} is selected from hydrogen, hydroxy, amino, and halogen; wherein said C₃-C₁₀-cycloalkyl and 3- to 14-membered heterocyclyl are optionally substituted with 1-2 halogen substituents;
R⁵ is selected from hydrogen, hydroxy, halogen, cyano, C₁-C₆-alkyl, C₁-C₆-alkoxy, halo-C₁-C₆-alkyl, hydroxy-C₁-C₆-alkyl, hydroxy-halo-C₁-C₆-alkyl, carboxy-C₁-C₆-alkyl, halo-C₁-C₆-alkoxy, oxo, NH₂—SO₂—, amino, C₁-C₆-alkyl-NH—, (C₁-C₆-alkyl)₂N—, carbamoyl, C₁-C₆-alkyl-NH—C(O)—, (C₁-C₆-alkyl)₂N—C(O)—, carboxy, C₁-C₆-alkoxycarbonyl, C₁-C₆-alkoxycarbonyl-C₁-C₆-alkyl-, (C₁-C₆-alkyl)₂P(O)—, (C₁-C₆-alkyl)₂P(O)—C₁-C₆-alkyl-, a group

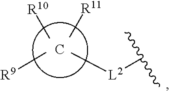

and a group

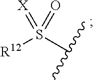

R⁶ is selected from hydrogen, halogen, cyano, oxo, C₁-C₆-alkoxy, halo-C₁-C₆-alkoxy, halo-C₁-C₆-alkyl, and C₁-C₆-alkyl-SO₂—;
R⁷ is selected from hydrogen and C₁-C₆-alkyl;
R^{8a} is selected from hydrogen, halogen, and carbamoyl;
R^{8b} is selected from hydrogen and halogen;
R⁹ is selected from hydrogen, halogen, cyano, hydroxy, oxo, carboxy, C₁-C₆-alkoxycarbonyl, amino, C₁-C₆-alkyl-NH—, (C₁-C₆-alkyl)₂N—, carbamoyl, C₁-C₆-alkyl-NH—C(O)—, C₁-C₆-alkyl, hydroxy-C₁-C₆-alkyl, halo-C₁-C₆-alkyl, C₁-C₆-alkoxy-C₁-C₆-alkyl, C₁-C₆-alkoxy, halo-C₁-C₆-alkoxy, C₃-C₁₀-cycloalkyl, cyano, C₁-C₆-alkyl-SO₂—, halo-C₁-C₆-alkyl-SO₂—, and 5- to 14-membered heteroaryl; wherein said C₃-C₁₀-cycloalkyl and 5- to 14-membered heteroaryl is optionally substituted with one substituent selected from C₁-C₆-alkyl and halo-C₁-C₆-alkyl; or
R⁹ and R⁵ together form a group

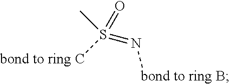

R¹⁰ is selected from hydrogen, halogen, C₁-C₆-alkyl, halo-C₁-C₆-alkyl, and oxo;
R¹¹ is selected from hydrogen and halogen;
R¹² is selected from amino, C₁-C₆-alkyl and halo-C₁-C₆-alkyl;
R^{13a} is selected from hydrogen, C₁-C₆-alkyl, C₃-C₁₀-cycloalkyl, C₃-C₁₀-cycloalkyl-C₁-C₆-alkyl-;
R^{13b} is hydrogen; and
R^{13c} is selected from hydrogen and C₁-C₆-alkyl.

In one embodiment, the present invention provides a compound of formula (I) as described herein, or a pharmaceutically acceptable salt thereof, wherein A is selected from:

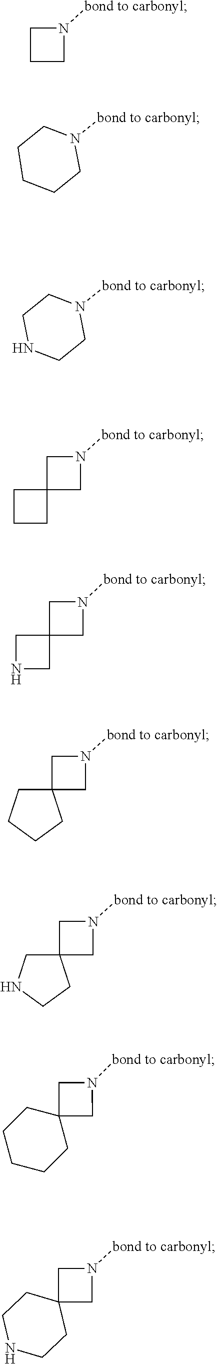

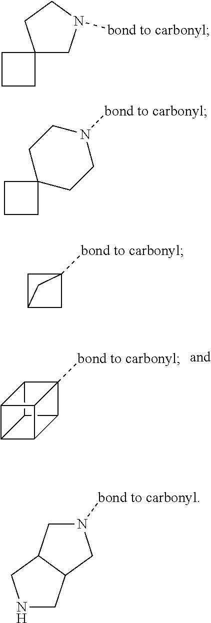

In a preferred embodiment, the present invention provides a compound of formula (I) as described herein, or a pharmaceutically acceptable salt thereof, wherein A is selected from:

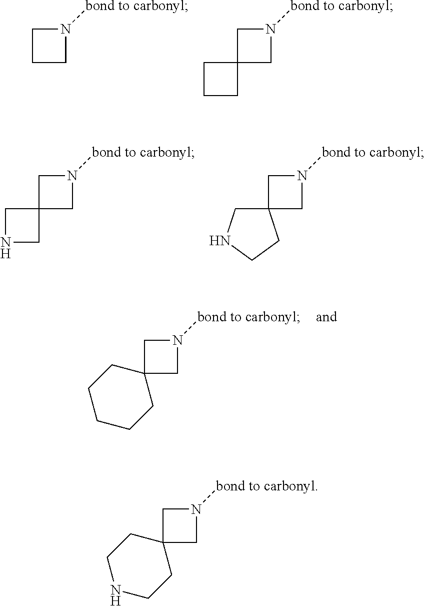

In one embodiment, the present invention provides a compound of formula (I) as described herein, or a pharmaceutically acceptable salt thereof, wherein:
B is selected from C₆-C₁₄-aryl, C₃-C₁₀-cycloalkyl, 5- to 14-membered heteroaryl, and 3- to 14-membered heterocyclyl;
C is selected from C₆-C₁₄-aryl, C₃-C₁₀-cycloalkyl, 3- to 14-membered heterocyclyl, and 5- to 14-membered heteroaryl;
L¹ is selected from a covalent bond, carbonyl, —CR^{8a}R^{8b}—, —CH₂O—, —OCH₂—, —CH₂NR^{13a}, —NR^{13b}CH₂—, —CH₂CH₂—, —CH₂NR^{13c}CH₂—, —CH₂NHCO—, —O—, —NH—, —SO₂NH—, —NHSO₂—, —SO₂NHCH₂—, —CH₂NHSO₂—, —SO₂—, —NHC(O)—, —C(O)NH—, and

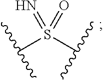 [SMILES: *S(*)(=N)=O]

L² is selected from a covalent bond, —CR^{8a}R^{8b}—, —NH—, —N(C₁-C₆-alkyl)-, —O—, —CH₂NH—, —NHCH₂—, —CH₂O—, —OCH₂—, —SO₂—, and —CH₂SO₂—;
X is NH or O;
R¹ is selected from a group

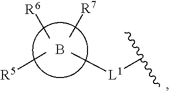

C₁-C₆-alkyl-SO₂—NH—, C₁-C₆-alkyl-SO₂—, and halo-C₁-C₆-alkyl-C(O)—;
R² is selected from hydrogen and hydroxy;
R⁵ is selected from hydrogen, hydroxy, halogen, cyano, C₁-C₆-alkyl, C₁-C₆-alkoxy, halo-C₁-C₆-alkyl, hydroxy-C₁-C₆-alkyl, hydroxy-halo-C₁-C₆-alkyl, carboxy-C₁-C₆-alkyl, halo-C₁-C₆-alkoxy, oxo, NH₂—SO₂—, C₁-C₆-alkyl-NH—, carbamoyl, C₁-C₆-alkyl-NH—C(O)—, carboxy, C₁-C₆-alkoxycarbonyl, C₁-C₆-alkoxycarbonyl-C₁-C₆-alkyl-, (C₁-C₆-alkyl)₂P(O)—, (C₁-C₆-alkyl)₂P(O)—C₁-C₆-alkyl-, a group

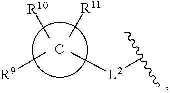

and a group

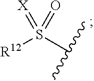

R⁶ is selected from hydrogen, halogen, cyano, oxo, C₁-C₆-alkoxy, halo-C₁-C₆-alkoxy, halo-C₁-C₆-alkyl, and C₁-C₆-alkyl-SO₂—;
R⁷ is selected from hydrogen and C₁-C₆-alkyl;
R^{8a} is selected from hydrogen, halogen, C₁-C₆-alkyl, and carbamoyl;
R^{8b} is selected from hydrogen and halogen;
R⁹ is selected from hydrogen, halogen, cyano, hydroxy, oxo, carboxy, C₁-C₆-alkoxycarbonyl, C₁-C₆-alkyl-NH—, carbamoyl, C₁-C₆-alkyl-NH—C(O)—, C₁-C₆-alkyl, hydroxy-C₁-C₆-alkyl, halo-C₁-C₆-alkyl, C₁-C₆-alkoxy-C₁-C₆-alkyl, C₁-C₆-alkoxy, halo-C₁-C₆-alkoxy, C₃-C₁₀-cycloalkyl, cyano, C₁-C₆-alkyl-SO₂—, halo-C₁-C₆-alkyl-SO₂—, and 5- to 14-membered heteroaryl; wherein said C₃-C₁₀-cycloalkyl and 5- to 14-membered heteroaryl is optionally substituted with one substituent selected from C₁-C₆-alkyl and halo-C₁-C₆-alkyl; or
R⁹ and R⁵ together form a group

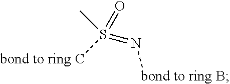

R¹⁰ is selected from hydrogen, halogen, C₁-C₆-alkyl, halo-C₁-C₆-alkyl, and oxo;
R¹¹ is selected from hydrogen and halogen;
R¹² is selected from amino, C₁-C₆-alkyl and halo-C₁-C₆-alkyl;
R¹³ is selected from hydrogen, C₁-C₆-alkyl, C₃-C₁₀-cycloalkyl, C₃-C₁₀-cycloalkyl-C₁-C₆-alkyl-;
R^{13b} is hydrogen; and
R^{13c} is selected from hydrogen and C₁-C₆-alkyl.

In one embodiment, the present invention provides a compound of formula (I) as described herein, or a pharmaceutically acceptable salt thereof, wherein:
B is selected from C₆-C₁₄-aryl, C₃-C₁₀-cycloalkyl, 5- to 14-membered heteroaryl, and 3- to 14-membered heterocyclyl;
C is selected from C₆-C₁₄-aryl, C₃-C₁₀-cycloalkyl, 3- to 14-membered heterocyclyl, and 5- to 14-membered heteroaryl;
L¹ is selected from a covalent bond, carbonyl, —CR^{8a}R^{8b}—, —CH₂O—, —OCH₂—, —CH₂NR^{13a}—, —NR^{13b}CH₂—, —CH₂CH₂—, —CH₂NR^{13c}CH₂—, —CH₂NHCO—, —O—, —NH—, —SO₂NH—, —NHSO₂—, —SO₂NHCH₂—, —CH₂NHSO₂—, —SO₂—, —NHC(O)—, —C(O)NH—, and

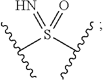 [SMILES: *S(*)(=N)=O]

L² is selected from a covalent bond, —CR^{8a}R^{8b}—, —NH—, —N(C₁-C₆-alkyl)-, —O—, —CH₂NH—, —NHCH₂—, —CH₂O—, —SO₂—, and —CH₂SO₂—;
X is NH or O;
R¹ is selected from a group

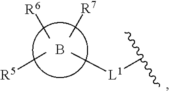

C₁-C₆-alkyl-SO₂—NH—, C₁-C₆-alkyl-SO₂—, and halo-C₁-C₆-alkyl-C(O)—;
R² is selected from hydrogen and hydroxy;
R⁵ is selected from hydrogen, hydroxy, halogen, cyano, C₁-C₆-alkyl, C₁-C₆-alkoxy, halo-C₁-C₆-alkyl, hydroxy-C₁-C₆-alkyl, hydroxy-halo-C₁-C₆-alkyl, carboxy-C ₁-C₆-alkyl, halo-C₁-C₆-alkoxy, oxo, NH₂—SO₂—, C₁-C₆-alkyl-NH—, carbamoyl, C₁-C₆-alkyl-NH—C(O)—, carboxy, C₁-C₆-alkoxycarbonyl, C₁-C₆-alkoxycarbonyl-C₁-C₆-alkyl-, (C₁-C₆-alkyl)₂P(O)—, (C₁-C₆-alkyl)₂P(O)—C₁-C₆-alkyl-, a group

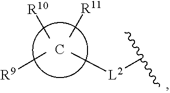

and a group

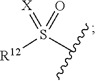

R⁶ is selected from hydrogen, halogen, cyano, oxo, C₁-C₆-alkoxy, halo-C₁-C₆-alkoxy, halo-C₁-C₆-alkyl, and C₁-C₆-alkyl-SO₂—;
R⁷ is selected from hydrogen and C₁-C₆-alkyl;
R^{8a} is selected from hydrogen, halogen and carbamoyl;
R^{8b} is selected from hydrogen and halogen;
R⁹ is selected from hydrogen, halogen, cyano, hydroxy, oxo, carboxy, C₁-C₆-alkoxycarbonyl, C₁-C₆-alkyl-NH—, carbamoyl, C₁-C₆-alkyl-NH—C(O)—, C₁-C₆-alkyl, hydroxy-C₁-C₆-alkyl, halo-C₁-C₆-alkyl, C₁-C₆-alkoxy-C₁-C₆-alkyl, C₁-C₆-alkoxy, halo-C₁-C₆-alkoxy, C₃-C₁₀-cycloalkyl, cyano, C₁-C₆-alkyl-SO₂—, halo-C₁-C₆-alkyl-SO₂—, and 5- to 14-membered heteroaryl; wherein said C₃-C₁₀-cycloalkyl and 5- to 14-membered heteroaryl is optionally substituted with one substituent selected from C₁-C₆-alkyl and halo-C₁-C₆-alkyl; or
R⁹ and R⁵ together form a group

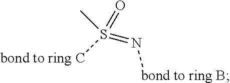

R¹⁰ is selected from hydrogen, halogen, C₁-C₆-alkyl, halo-C₁-C₆-alkyl, and oxo;
R¹¹ is selected from hydrogen and halogen;
R¹² is selected from amino, C₁-C₆-alkyl and halo-C₁-C₆-alkyl;
R^{13a} is selected from hydrogen, C₁-C₆-alkyl, C₃-C₁₀-cycloalkyl, C₃-C₁₀-cycloalkyl-C₁-C₆-alkyl-;
R^{13b} is hydrogen; and
R^{13c} is selected from hydrogen and C₁-C₆-alkyl.

In a preferred embodiment, the present invention provides a compound of formula (I) as described herein, or a pharmaceutically acceptable salt thereof, wherein:
B is selected from C₆-C₁₄-aryl, C₃-C₁₀-cycloalkyl, 5- to 14-membered heteroaryl, and 3- to 14-membered heterocyclyl;
C is selected from C₃-C₁₀-cycloalkyl and 5- to 14-membered heteroaryl;
L¹ is selected from a covalent bond, —CR^{8a}R^{8b}—, and —CH₂O—;
L² is selected from a covalent bond, —NH—, and —CH₂NH—;
X is NH or O;
R¹ is a group

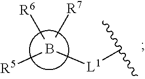

R² is hydrogen;
R⁵ is selected from halogen, cyano, halo-C₁-C₆-alkyl, oxo, halo-C₁-C₆-alkoxy, C₁-C₆-alkyl-SO₂—, halo-C₁-C₆-alkyl-SO₂—, a group

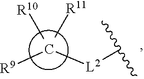

and a group

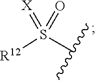

R⁶ is selected from hydrogen, halogen, cyano, and halo-C₁-C₆-alkyl;
R⁷ is selected from hydrogen and C₁-C₆-alkyl;
R^{8a} is selected from hydrogen and halogen;
R^{8b} is selected from hydrogen and halogen;
R⁹ is selected from hydrogen, C₁-C₆-alkyl and halo-C₁-C₆-alkyl;
R¹⁰ is selected from hydrogen, C₁-C₆-alkyl, and halo-C₁-C₆-alkyl;
R¹¹ is hydrogen; and
R¹² is halo-C₁-C₆-alkyl.

In a particularly preferred embodiment, the present invention provides a compound of formula (I) as described herein, or a pharmaceutically acceptable salt thereof, wherein:
B is selected from phenyl, bicyclo[1.1.1]pentyl, pyridyl, oxazolyl, pyrimidinyl, pyrazinyl, pyridazinyl, 1,2,4-thiadiazolyl, pyrazolyl, imidazolyl, 1H-1,2,4-triazolyl, triazol-2-yl, 2H-triazolyl, isoxazolyl, 1,2,4-oxadiazolyl, 1,3,4-oxadiazolyl, 1,2-dihydropyridyl, and 1,2-dihydropyrazinyl;
C is selected from cyclopropyl, pyrazolyl, 1,3,4-oxadiazolyl, 1,3,4-thiadiazolyl, and pyrazinyl;
L¹ is selected from a covalent bond, —CR^{8a}R^{8b}—, and —CH₂O—;
L² is selected from a covalent bond, —NH—, and —CH₂NH—;
X is NH or O;
R¹ is a group

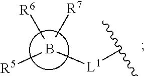

R² is hydrogen;
R⁵ is selected from chloro, fluoro, cyano, CF₃, 2,2,2-trifluoroethyl, CF₃O, oxo, methylsulfonyl, CF₃-SO₂—, a group

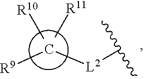

and a group

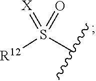

R⁶ is selected from hydrogen, fluoro, cyano, and CF₃;
R⁷ is selected from hydrogen and methyl;
R^{8a} is selected from hydrogen and fluoro;
R^{8b} is selected from hydrogen and fluoro;
R⁹ is selected from hydrogen, methyl, 2,2-dimethylpropyl, CHF₂, and CF₃;
R¹⁰ is selected from hydrogen, methyl, and CF₃;
R¹¹ is hydrogen; and
R¹² is CF₃.

In a particularly preferred embodiment, the present invention provides a compound of formula (I) as described herein, or a pharmaceutically acceptable salt thereof, wherein:
B is selected from phenyl, bicyclo[1.1.1]pentyl, pyridyl, oxazolyl, pyrimidinyl, pyrazinyl, pyridazinyl, 1,2,4-thiadiazolyl, pyrazolyl, imidazolyl, 1H-1,2,4-triazolyl, triazol-2-yl, 2H-triazolyl, isoxazolyl, 1,2,4-oxadiazolyl, 1,3,4-oxadiazolyl, and 1,2-dihydropyridyl;
C is selected from cyclopropyl, pyrazolyl, 1,3,4-oxadiazolyl, 1,3,4-thiadiazolyl, and pyrazinyl;
L¹ is selected from a covalent bond, —CR^{8a}R^{8b}—, and —CH₂O—;
L² is selected from a covalent bond, —NH—, and —CH₂NH—;
X is NH or O;
R¹ is a group

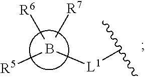

R² is hydrogen;
R⁵ is selected from chloro, fluoro, cyano, CF₃, CF₃O, oxo, methylsulfonyl, CF₃-SO₂—, a group

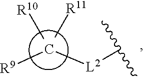

and a group

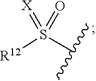

R⁶ is selected from hydrogen, fluoro, cyano, and CF₃;
R⁷ is selected from hydrogen and methyl;
R^{8a} is selected from hydrogen and fluoro;
R^{8b} is selected from hydrogen and fluoro;
R⁹ is selected from hydrogen, methyl, 2,2-dimethylpropyl, CHF₂, and CF₃;
R¹⁰ is selected from hydrogen, methyl, and CF₃;
R¹¹ is hydrogen; and
R¹² is CF₃.

In one embodiment, the present invention provides a compound of formula (I) as described herein, or a pharmaceutically acceptable salt thereof, wherein:
A is selected from:

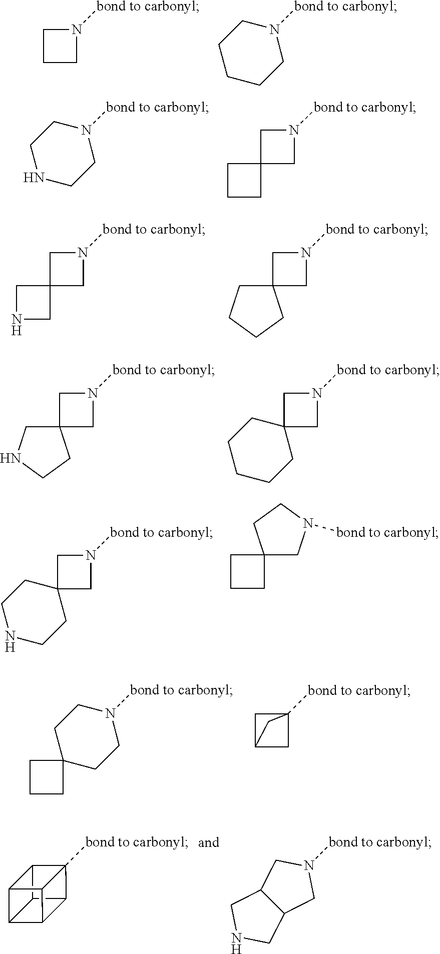

B is selected from C₆-C₁₄-aryl, C₃-C₁₀-cycloalkyl, 5- to 14-membered heteroaryl, and 3- to 14-membered heterocyclyl;
C is selected from C₆-C₄-aryl, C₃-C₁₀-cycloalkyl, 3- to 14-membered heterocyclyl, and 5- to 14-membered heteroaryl;
L¹ is selected from a covalent bond, carbonyl, —CR^{8a}R^{8b}—, —CH₂O—, —OCH₂—, —CH₂NR^{13a}—, —NR^{13b}CH₂—, —CH₂CH₂—, CH₂NHCH₂—, —CH₂NHCO—, —O—, —NH—, —SO₂NH—, NHSO₂—, —SO₂NHCH₂—, —CH₂NHSO₂—, —SO₂—, —NHC(O)—, —C(O)NH—, and

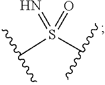 [SMILES: *S(*)(=N)=O]

L² is selected from a covalent bond, —CR^{8a}R^{8b}—, —NH—, —N(C₁-C₆-alkyl)-, —O—, —CH₂NH—, —NHCH₂—, —CH₂O—, —OCH₂—, —SO₂—, and —CH₂SO₂—;
X is NH or O;
R¹ is selected from a group

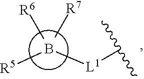

C₁-C₆-alkyl-SO₂—NH—, C₁-C₆-alkyl-SO₂—, and halo-C₁-C₆-alkyl-C(O)—;
R² is selected from hydrogen and hydroxy;
R³ is hydrogen;
R⁴ is selected from hydrogen and a group —C(R^{4a}R^{4b}R^{4c});
R^{4a} is halo-C₁-C₆-alkyl; R^{4b} is hydroxy; and R is hydrogen; or
R^{4a}, R^{4b}, and R^{4c} are each independently halogen; or
R^{4a} and R^{4b}, taken together with the carbon atom to which they are attached, form a C₃-C₁₀-cycloalkyl or a 3- to 14-membered heterocyclyl; and R¹ is selected from hydrogen, hydroxy, amino, and halogen; wherein said C₃-C₁₀-cycloalkyl and 3- to 14-membered heterocyclyl are optionally substituted with 1-2 halogen substituents;
R⁵ is selected from hydrogen, hydroxy, halogen, cyano, C₁-C₆-alkyl, C₁-C₆-alkoxy, halo-C₁-C₆-alkyl, hydroxy-C₁-C₆-alkyl, hydroxy-halo-C₁-C₆-alkyl, carboxy-C₁-C₆-alkyl, halo-C₁-C₆-alkoxy, oxo, NH₂—SO₂—, C₁-C₆-alkyl-NH—, carbamoyl, C₁-C₆-alkyl-NH—C(O)—, carboxy, C₁-C₆-alkoxycarbonyl, C₁-C₆-alkoxycarbonyl-C₁-C₆-alkyl-, (C₁-C₆-alkyl)₂P(O)—, (C₁-C₆-alkyl)₂P(O)—C₁-C₆-alkyl-, a group

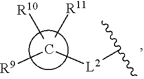

and a group

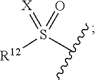

R⁶ is selected from hydrogen, halogen, cyano, oxo, C₁-C₆-alkoxy, halo-C₁-C₆-alkoxy, halo-C₁-C₆-alkyl, and C₁-C₆-alkyl-SO₂—;
R⁷ is selected from hydrogen and C₁-C₆-alkyl;
R^{8a} is selected from hydrogen, halogen, C₁-C₆-alkyl, and carbamoyl;
R^{8b} is selected from hydrogen and halogen;
R⁹ is selected from hydrogen, halogen, cyano, hydroxy, oxo, carboxy, C₁-C₆-alkoxycarbonyl, C₁-C₆-alkyl-NH—, carbamoyl, C₁-C₆-alkyl-NH—C(O)—, C₁-C₆-alkyl, hydroxy-C₁-C₆-alkyl, halo-C₁-C₆-alkyl, C₁-C₆-alkoxy-C₁-C₆-alkyl, C₁-C₆-alkoxy, halo-C₁-C₆-alkoxy, C₃-C₁₀-cycloalkyl, cyano, C₁-C₆-alkyl-SO₂—, halo-C₁-C₆-alkyl-SO₂—, and 5- to 14-membered heteroaryl; wherein said C₃-C₁₀-cycloalkyl and 5- to 14-membered heteroaryl is optionally substituted with one substituent selected from C₁-C₆-alkyl and halo-C₁-C₆-alkyl; or
R⁹ and R⁵ together form a group

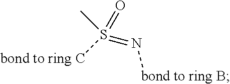

R¹⁰ is selected from hydrogen, halogen, C₁-C₆-alkyl, halo-C₁-C₆-alkyl, and oxo;
R¹¹ is selected from hydrogen and halogen;
R¹² is selected from amino, C₁-C₆-alkyl and halo-C ₁-C₆-alkyl;
R^{13a} is selected from hydrogen, C₁-C₆-alkyl, C₃-C₁₀-cycloalkyl, C₃-C₁₀-cycloalkyl-C ₁-C₆-alkyl-;
R^{13b} is hydrogen; and
R^{13c} is selected from hydrogen and C₁-C₆-alkyl.

In one embodiment, the present invention provides a compound of formula (I) as described herein, or a pharmaceutically acceptable salt thereof, wherein:
A is selected from:

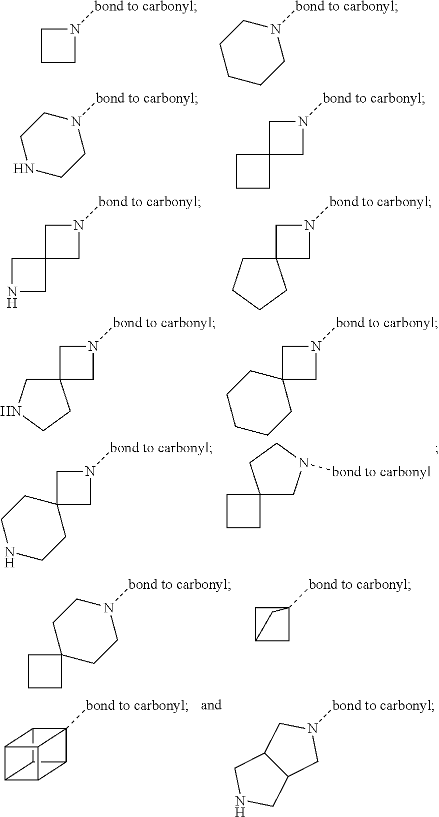

B is selected from C₆-C₁₄-aryl, C₃-C₁₀-cycloalkyl, 5- to 14-membered heteroaryl, and 3- to 14-membered heterocyclyl;
C is selected from C₆-C₁₄-aryl, C₃-C₁₀-cycloalkyl, 3- to 14-membered heterocyclyl, and 5- to 14-membered heteroaryl;
L¹ is selected from a covalent bond, carbonyl, —CR^{8a}R^{8b}—, —CH₂O—, —OCH₂—, —CH₂NR^{13a}—, —NR^{13b}CH₂—, —CH₂CH₂—, —CH₂NR^{13c}CH—, —CH₂NHCO—, —O—, —NH—, —SO₂NH—, —NHSO₂—, —SO₂NHCH₂—, —CH₂NHSO₂—, —SO₂—, —NHC(O)—, and

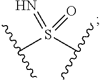 [SMILES: *S(*)(=N)=O]

L² is selected from a covalent bond, —CR^{8a}R^{8b}—, NH—, —N(C₁-C₆-alkyl)-, —O—, —CH₂NH—, —NHCH₂—, —CH₂O—, —SO₂—, and —CH₂SO₂—;
X is NH or O;
R¹ is selected from a group

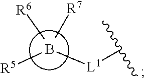

C₁-C₆-alkyl-SO₂—NH—, C₁-C₆-alkyl-SO₂—, and halo-C ₁-C₆-alkyl-C(O)—;
R² is selected from hydrogen and hydroxy;
R³ is hydrogen;
R⁴ is selected from hydrogen and a group
R^{4a} is halo-C₁-C₆-alkyl; R^{4b} is hydroxy; and R is hydrogen; or
R^{4a}, R^{4b}, and R^{4c} are each independently halogen; or
R^{4a} and R^{4b}, taken together with the carbon atom to which they are attached, form a C₃-C₁₀-cycloalkyl or a 3- to 14-membered heterocyclyl; and R is selected from hydrogen, hydroxy, amino, and halogen; wherein said C ₄-C₁₀-cycloalkyl and 3- to 14-membered heterocyclyl are optionally substituted with 1-2 halogen substituents;
R⁵ is selected from hydrogen, hydroxy, halogen, cyano, C₁-C₆-alkyl, C₁-C₆-alkoxy, halo-C₁-C₆-alkyl, hydroxy-C₁-C₆-alkyl, hydroxy-halo-C₁-C₆-alkyl, carboxy-C₁-C₆-alkyl, halo-C₁-C₆-alkoxy, oxo, NH₂—SO₂—, C₁-C₆-alkyl-NH—, carbamoyl, C₁-C₆-alkyl-NH—C(O)—, carboxy, C₁-C₆-alkoxycarbonyl, C₁-C₆-alkoxycarbonyl-C₁-C₆-alkyl-, (C₁-C₆-alkyl)₂P(O)—, (C₁-C₆-alkyl)₂P(O)—C₁-C₆-alkyl-, a group

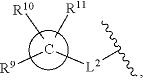

and a group

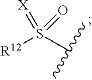

R⁶ is selected from hydrogen, halogen, cyano, oxo, C₁-C₆-alkoxy, halo-C₁-C₆-alkoxy, halo-C₁-C₆-alkyl, and C₁-C₆-alkyl-SO₂—;
R⁷ is selected from hydrogen and C₁-C₆-alkyl;
R^{8a} is selected from hydrogen, halogen and carbamoyl;
R^{8b} is selected from hydrogen and halogen;
R⁹ is selected from hydrogen, halogen, cyano, hydroxy, oxo, carboxy, C₁-C₆-alkoxycarbonyl, C₁-C₆-alkyl-NH—, carbamoyl, C₁-C₆-alkyl-NH—C(O)—, C₁-C₆-alkyl, hydroxy-C₁-C₆-alkyl, halo-C₁-C₆-alkyl, C₁-C₆-alkoxy-C₁-C₆-alkyl, C₁-C₆-alkoxy, halo-C₁-C₆-alkoxy, C₃-C₁₀-cycloalkyl, cyano, C₁-C₆-alkyl-SO₂—, halo-C₁-C₆-alkyl-SO₂—, and 5- to 14-membered heteroaryl; wherein said C₃-C₁₀-cycloalkyl and 5- to 14-membered heteroaryl is optionally substituted with one substituent selected from C₁-C₆-alkyl and halo-C₁-C₆-alkyl; or
R⁹ and R⁵ together form a group

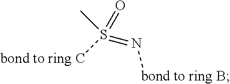

R¹⁰ is selected from hydrogen, halogen, C₁-C₆-alkyl, halo-C₁-C₆-alkyl, and oxo;
R¹¹ is selected from hydrogen and halogen; R¹² is selected from amino, C₁-C₆-alkyl and halo-C₁-C₆-alkyl;
R^{13a} is selected from hydrogen, C₁-C₆-alkyl, C₃-C₁₀-cycloalkyl, C₃-C₁₀-cycloalkyl-C₁-C₆-alkyl-;
R^{13b} is hydrogen; and
R^{13c} is selected from hydrogen and C₁-C₆-alkyl.

In a preferred embodiment, the present invention provides a compound of formula (I) as described herein, or a pharmaceutically acceptable salt thereof, wherein:
A is selected from:

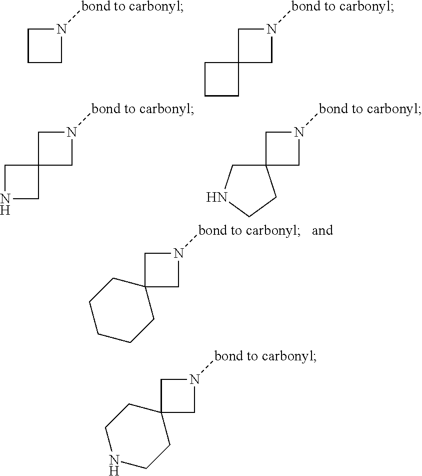

B is selected from C₆-C₁₄-aryl, C₃-C₁₀-cycloalkyl, 5- to 14-membered heteroaryl, and 3- to 14-membered heterocyclyl;
C is selected from C₃-C₁₀-cycloalkyl and 5- to 14-membered heteroaryl;
L¹ is selected from a covalent bond, —CR^{8a}R^{8b}, and —CH₂O—;
L² is selected from a covalent bond, —NH—, and —CH₂NH—;
X is NH or O;
R¹ is a group

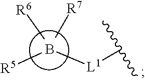

R² is hydrogen;
R³ is hydrogen;
R⁴ is a group —C(R^{4a}R^{4b}R^{4c});
R^{4a}, R^{4b}, and R^{4c} are each independently halogen; or
R^{4a} and R^{4b}, taken together with the carbon atom to which they are attached, form a C₃-C₁₀-cycloalkyl; and R^{4c} is selected from hydrogen, hydroxy, and halogen; wherein said C₃-C₁₀-cycloalkyl is optionally substituted with 1-2 halogen substituents;
R⁵ is selected from halogen, cyano, halo-C₁-C₆-alkyl, halo-C₁-C₆-alkoxy, C₁-C₆-alkyl-SO₂—, halo-C₁-C₆-alkyl-SO₂—, oxo, a group

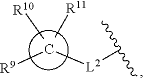

and a group

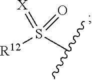

R⁶ is selected from hydrogen, halogen, cyano, and halo-C₁-C₆-alkyl;
R⁷ is selected from hydrogen and C₁-C₆-alkyl;
R^{8a} is selected from hydrogen and halogen;
R^{8b} is selected from hydrogen and halogen;
R⁹ is selected from hydrogen, C I-C₆-alkyl and halo-C ₁-C₆-alkyl;
R¹⁰ is selected from hydrogen, C₁-C₆-alkyl, and halo-C₁-C₆-alkyl;
R¹¹ is hydrogen; and
R¹² is halo-C₁-C₆-alkyl.

In a particularly preferred embodiment, the present invention provides a compound of formula (I) as described herein, or a pharmaceutically acceptable salt thereof, wherein:
A is selected from:

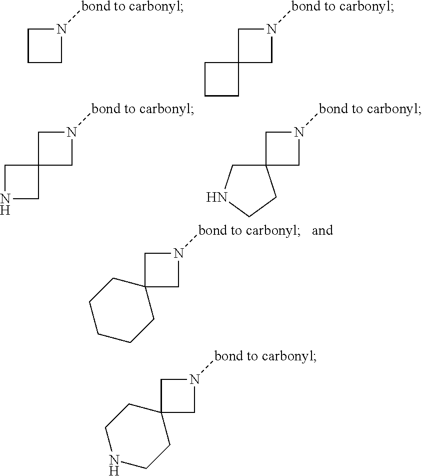

B is selected from phenyl, bicyclo[1.1.1]pentanyl, pyridyl, oxazolyl, pyrimidinyl, pyrazinyl, pyridazinyl, 1,2,4-thiadiazolyl, pyrazolyl, imidazolyl, 1H-1,2,4-triazolyl, triazol-2-yl, 2H-triazolyl, isoxazolyl, 1,2,4-oxadiazolyl, 1,3,4-oxadiazolyl, 1,2-dihydropyridyl, and 1,2-dihydropyrazinyl;
C is selected from cyclopropyl, pyrazolyl, 1,3,4-oxadiazole, pyrazinyl, and 1,3,4-thiadiazolyl;
L¹ is selected from a covalent bond, —CR^{8a}R^{8b}—, and —CH₂O—;
L² is selected from a covalent bond, —NH—, and —CH₂NH—;
X is NH or O;

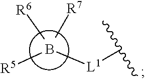

R¹ is a group
R² is hydrogen;
R³ is hydrogen;
R⁴ is a group —C(R^{4a}R^{4b}R^{4c});
R^{4a}, R^{4b}, and R^{4c} are each independently fluoro; or
R^{4a} and R^{4b}, taken together with the carbon atom to which they are attached, form a cyclopropyl; and R^{4c} is selected from hydrogen, hydroxy, and fluoro; wherein said cyclopropyl is optionally substituted with 1-2 fluoro substituents;
R⁵ is selected from chloro, fluoro, cyano, CF₃, 2,2,2-trifluoroethyl, CF₃O, methylsulfonyl, CF₃—SO₂—, oxo, a group

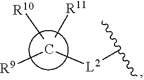

and a group

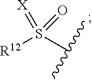

R⁶ is selected from hydrogen, fluoro, cyano, and CF₃;
R⁷ is selected from hydrogen and methyl;
R^{8a} is selected from hydrogen and fluoro;
R^{8b} is selected from hydrogen and fluoro;
R⁹ is selected from hydrogen, methyl, 2,2-dimethylpropyl, CHF₂ and CF₃;
R¹⁰ is selected from hydrogen, methyl, and CF₃;
R¹¹ is hydrogen; and
R¹² is CF₃.

In a particularly preferred embodiment, the present invention provides a compound of formula (I) as described herein, or a pharmaceutically acceptable salt thereof, wherein:
A is selected from:

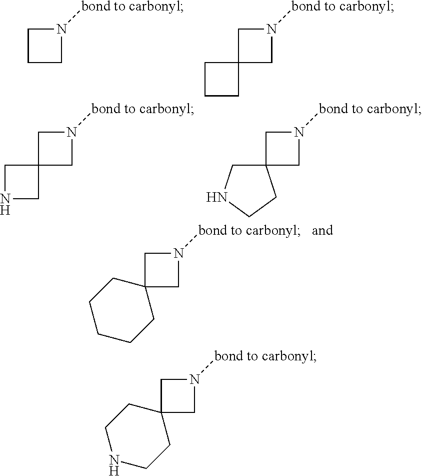

B is selected from phenyl, bicyclo[1.1.1]pentanyl, pyridyl, oxazolyl, pyrimidinyl, pyrazinyl, pyridazinyl, 1,2,4-thiadiazolyl, pyrazolyl, imidazolyl, 1H-1,2,4-triazolyl, isoxazolyl, 1,2,4-oxadiazolyl, 1,3,4-oxadiazolyl, and 1,2-dihydropyridyl;
C is selected from cyclopropyl, pyrazolyl, 1,3,4-oxadiazole, pyrazinyl, and 1,3,4-thiadiazolyl;
L¹ is selected from a covalent bond, —CR^{8a}R^{8b}—, and —CH₂O—;
L² is selected from a covalent bond, —NH—, and —CH₂NH—;
X is NH or O;
R¹ is a group

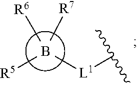

R² is hydrogen;
R³ is hydrogen;
R⁴ is a group
R^{4a}, R^{4b}, and R^{4c} are each independently fluoro; or
R^{4a} and R^{4b}, taken together with the carbon atom to which they are attached, form a cyclopropyl; and R^{4c} is selected from hydrogen, hydroxy, and fluoro; wherein said cyclopropyl is optionally substituted with 1-2 fluoro substituents;
R⁵ is selected from chloro, fluoro, CF₃, CF₃O, methylsulfonyl, CF₃-SO₂—, oxo, a group

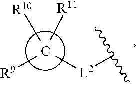

and a group

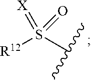

R⁶ is selected from hydrogen, fluoro, cyano, and CF₃;
R⁷ is selected from hydrogen and methyl;
R^{8a} is selected from hydrogen and fluoro;
R^{8b} is selected from hydrogen and fluoro;
R⁹ is selected from methyl, 2,2-dimethylpropyl, CHF₂ and CF₃;
R¹⁰ is selected from hydrogen, methyl, and CF₃;
R¹¹ is hydrogen; and
R¹² is CF₃.

In a particularly preferred embodiment, the present invention provides a compound of formula (I) as described herein, or a pharmaceutically acceptable salt thereof, wherein:
A is

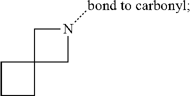

B is a 5- to 6-membered heteroaryl;
L¹ is —CR^{8a}R^{8b}—;
R¹ is a group

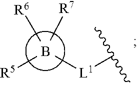

R² is hydrogen;
R³ is hydrogen;
R⁴ is a group —C(R^{4a}R^{4b}R^{4c});
R^{4a} and R^{4b}, taken together with the carbon atom to which they are attached, form a cyclopropyl; and R is hydrogen;
R⁵ is halo-C₁-C₆-alkyl;
R⁶ is hydrogen;
R⁷ is hydrogen;
R^{8a} is hydrogen; and
R^{8b} is hydrogen.

In one embodiment, the present invention provides a compound of formula (I) as described herein, or a pharmaceutically acceptable salt thereof, wherein B is a 5- to 9-membered heteroaryl.

In a preferred embodiment, the present invention provides a compound of formula (I) as described herein, or a pharmaceutically acceptable salt thereof, wherein B is a 5- to 6-membered heteroaryl.

In a preferred embodiment, the present invention provides a compound of formula (I) as described herein, or a pharmaceutically acceptable salt thereof, wherein B is a 5-membered heteroaryl.

In preferred embodiment, the present invention provides a compound of formula (I) as described herein, or a pharmaceutically acceptable salt thereof, wherein B is a 6-membered heteroaryl.

In a particularly preferred embodiment, the present invention provides a compound of formula (I) as described herein, or a pharmaceutically acceptable salt thereof, wherein B is selected from phenyl, bicyclo[1.1.1]pentyl, pyridyl, oxazolyl, pyrimidinyl, pyrazinyl, pyridazinyl, 1,2,4-thiadiazolyl, pyrazolyl, imidazolyl, 1H-1,2,4-triazolyl, triazol-2-yl, 2H-triazolyl, isoxazolyl, 1,2,4-oxadiazolyl, 1,3,4-oxadiazolyl, 1,2-dihydropyridyl, and 1,2-dihydropyrazinyl.

In a particularly preferred embodiment, the present invention provides a compound of formula (I) as described herein, or a pharmaceutically acceptable salt thereof, wherein B is selected from pyrimidinyl, pyrazinyl, and 2H-triazolyl.

In a particularly preferred embodiment, the present invention provides a compound of formula (I) as described herein, or a pharmaceutically acceptable salt thereof, wherein B is phenyl.

In a particularly preferred embodiment, the present invention provides a compound of formula (I) as described herein, or a pharmaceutically acceptable salt thereof, wherein B is bicyclo[1.1.1]pentyl.

In a particularly preferred embodiment, the present invention provides a compound of formula (I) as described herein, or a pharmaceutically acceptable salt thereof, wherein B is oxazolyl.

In a particularly preferred embodiment, the present invention provides a compound of formula (I) as described herein, or a pharmaceutically acceptable salt thereof, wherein B is pyrazinyl.

In a particularly preferred embodiment, the present invention provides a compound of formula (I) as described herein, or a pharmaceutically acceptable salt thereof, wherein B is pyridazinyl.

In a particularly preferred embodiment, the present invention provides a compound of formula (I) as described herein, or a pharmaceutically acceptable salt thereof, wherein B is pyrazolyl.

In a particularly preferred embodiment, the present invention provides a compound of formula (I) as described herein, or a pharmaceutically acceptable salt thereof, wherein B is imidazolyl.

In a particularly preferred embodiment, the present invention provides a compound of formula (I) as described herein, or a pharmaceutically acceptable salt thereof, wherein B is 1H-1,2,4-triazolyl.

In a particularly preferred embodiment, the present invention provides a compound of formula (I) as described herein, or a pharmaceutically acceptable salt thereof, wherein B is 2H-triazolyl.

In a particularly preferred embodiment, the present invention provides a compound of formula (I) as described herein, or a pharmaceutically acceptable salt thereof, wherein B is isoxazolyl.

In a particularly preferred embodiment, the present invention provides a compound of formula (I) as described herein, or a pharmaceutically acceptable salt thereof, wherein B is 1,2,4-oxadiazolyl.

In a particularly preferred embodiment, the present invention provides a compound of formula (I) as described herein, or a pharmaceutically acceptable salt thereof, wherein B is 1,3,4-oxadiazolyl.

In a particularly preferred embodiment, the present invention provides a compound of formula (I) as described herein, or a pharmaceutically acceptable salt thereof, wherein B is 1,2-dihydropyrazinyl.

In a particularly preferred embodiment, the present invention provides a compound of formula (I) as described herein, or a pharmaceutically acceptable salt thereof, wherein B is selected from pyridyl, 1,2-dihydropyridyl, pyrimidinyl, 1,2,4-thiadiazolyl, 1,2,3-oxadiazolyl, triazol-2-yl, and pyrazolyl.

In a particularly preferred embodiment, the present invention provides a compound of formula (I) as described herein, or a pharmaceutically acceptable salt thereof, wherein B is 1,2,3-oxadiazolyl.

In a particularly preferred embodiment, the present invention provides a compound of formula (I) as described herein, or a pharmaceutically acceptable salt thereof, wherein B is 1,2-dihydropyridyl.

In a particularly preferred embodiment, the present invention provides a compound of formula (I) as described herein, or a pharmaceutically acceptable salt thereof, wherein B is triazol-2-yl.

In a preferred embodiment, the present invention provides a compound of formula (I) as described herein, or a pharmaceutically acceptable salt thereof, wherein:
C is C₃-C₁₀-cycloalkyl;
R⁹ is halo-C₁-C₆-alkyl;
R¹⁰ is hydrogen; and
R¹¹ is hydrogen.

In a particularly preferred embodiment, the present invention provides a compound of formula (I) as described herein, or a pharmaceutically acceptable salt thereof, wherein:
C is cyclopropyl;
R⁹ is CF₃;
R¹⁰ is hydrogen; and
R¹¹ is hydrogen.

In one embodiment, the present invention provides a compound of formula (I) as described herein, or a pharmaceutically acceptable salt thereof, wherein L¹ is a covalent bond.

In one embodiment, the present invention provides a compound of formula (I) as described herein, or a pharmaceutically acceptable salt thereof, wherein
L¹ is —CR^{8a}R^{8b}—;
R^{8a} is selected from hydrogen and halogen; and
R^{8b} is selected from hydrogen and halogen.

In a preferred embodiment, the present invention provides a compound of formula (I) as described herein, or a pharmaceutically acceptable salt thereof, wherein
L¹ is —CR^{8a}R^{8b}—;
R^{8a} is selected from hydrogen and fluoro; and
R^{8b} is selected from hydrogen and fluoro.

In a particularly preferred embodiment, the present invention provides a compound of formula (I) as described herein, or a pharmaceutically acceptable salt thereof, wherein L¹ is —CH₂—.

In one embodiment, the present invention provides a compound of formula (I) as described herein, or a pharmaceutically acceptable salt thereof, wherein L¹ is —CH₂O—.

In a particularly preferred embodiment, the present invention provides a compound of formula (I) as described herein, or a pharmaceutically acceptable salt thereof, wherein:
L¹ is —CH₂—; and
B is a 5- to 6-membered heteroaryl.

In a particularly preferred embodiment, the present invention provides a compound of formula (I) as described herein, or a pharmaceutically acceptable salt thereof, wherein:
L¹ is —CH₂—; and
B is selected from pyrimidinyl, pyrazinyl, and 2H-triazolyl.

In one embodiment, the present invention provides a compound of formula (I) as described herein, or a pharmaceutically acceptable salt thereof, wherein:
L² is selected from a covalent bond, —CR^{8a}R^{8b}—, —NH—, N(C₁-C₆-alkyl)-, —O—, —CH₂NH—, —NHCH₂—, —CH₂O—, —OCH₂—, —SO₂—, and —CH₂SO₂—;
R^{8a} is selected from hydrogen, halogen, C₁-C₆-alkyl, and carbamoyl; and
R^{8b} is selected from hydrogen and halogen.

In a preferred embodiment, the present invention provides a compound of formula (I) as described herein, or a pharmaceutically acceptable salt thereof, wherein L² is a covalent bond.

In a preferred embodiment, the present invention provides a compound of formula (I) as described herein, or a pharmaceutically acceptable salt thereof, wherein R³ is hydrogen.

In one embodiment, the present invention provides a compound of formula (I) as described herein, or a pharmaceutically acceptable salt thereof, wherein
R⁴ is a group —C(R^{4a}R^{4b}R^{4c}); wherein
R^{4a}, R^{4b}, and R^{4c} are each independently halogen; or
R^{4a} and R^{4b}, taken together with the carbon atom to which they are attached, form a C₃-C₁₀-cycloalkyl; and R^{4c} is selected from hydrogen, hydroxy, and halogen; wherein said C₃-C₁₀-cycloalkyl is optionally substituted with 1-2 halogen substituents.

In a preferred embodiment, the present invention provides a compound of formula (I) as described herein, or a pharmaceutically acceptable salt thereof, wherein
R⁴ is a group C(R^{4a}R^{4b}R^{4c}); wherein
R^{4a}, R^{4b}, and R^{4c} are each independently halogen; or
R^{4a} and R^{4b}, taken together with the carbon atom to which they are attached, form a C₃-C₁₀-cycloalkyl; and R^{4c} is selected from hydrogen, hydroxy, and halogen; wherein said C₃-C₁₀-cycloalkyl is optionally substituted with 1-2 halogen substituents.

In a particularly preferred embodiment, the present invention provides a compound of formula (I) as described herein, or a pharmaceutically acceptable salt thereof, wherein:
R⁴ is a group —(R^{4a}R^{4b}R^{4c}); wherein
R^{4a}, R^{4b}, and R^{4c} are each independently fluoro; or
R^{4a} and R^{4b}, taken together with the carbon atom to which they are attached, form a cyclopropyl or a cyclobutyl; and R^{4c} is selected from hydrogen, hydroxy, and fluoro; wherein said cyclopropyl is optionally substituted with 1-2 fluoro substituents.

In one embodiment, the present invention provides a compound of formula (I) as described herein, or a pharmaceutically acceptable salt thereof, wherein R⁴ is selected from halo-C₁-C₆-alkyl and C₃-C₁₀-cycloalkyl, wherein said C₃-C₁₀-cycloalkyl is optionally substituted with 1 to 3 substituents selected from halogen and hydroxy.

In one embodiment, the present invention provides a compound of formula (I) as described herein, or a pharmaceutically acceptable salt thereof, wherein R⁴ is halo-C ₁-C₆-alkyl.

In a preferred embodiment, the present invention provides a compound of formula (I) as described herein, or a pharmaceutically acceptable salt thereof, wherein R⁴ is CF₃.

In one embodiment, the present invention provides a compound of formula (I) as described herein, or a pharmaceutically acceptable salt thereof, wherein R⁴ is C₃-C₁₀-cycloalkyl, wherein said C₃-C₁₀-cycloalkyl is optionally substituted with 1 to 3 substituents selected from halogen and hydroxy.

In a preferred embodiment, the present invention provides a compound of formula (I) as described herein, or a pharmaceutically acceptable salt thereof, wherein R⁴ is cyclopropyl or cyclobutyl, wherein said cyclopropyl or cyclobutyl is optionally substituted with 1 to 3 substituents selected from halogen and hydroxy.

In a particularly preferred embodiment, the present invention provides a compound of formula (I) as described herein, or a pharmaceutically acceptable salt thereof, wherein R⁴ is selected from CF₃, cyclopropyl, 1-fluorocycloproyl, 1-hydroxycyclopropyl, and 3,3-difluorocyclobutyl.

In a particularly preferred embodiment, the present invention provides a compound of formula (I) as described herein, or a pharmaceutically acceptable salt thereof, wherein R⁴ is

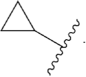 [SMILES: *C1CC1]

In a preferred embodiment, the present invention provides a compound of formula (I) as described herein, or a pharmaceutically acceptable salt thereof, wherein R⁵ is selected from halogen, cyano, halo-C₁-C₆-alkyl, oxo, halo-C₁-C₆-alkoxy, C₁-C₆-alkyl-SO₂—, halo-C₁-C₆-alkyl-SO₂—, a group

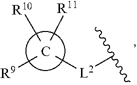

and a group

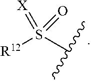

In a preferred embodiment, the present invention provides a compound of formula (I) as described herein, or a pharmaceutically acceptable salt thereof, wherein R⁵ is selected from halo-C₁-C₆-alkyl, a group

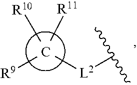

and a group

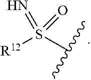

In a preferred embodiment, the present invention provides a compound of formula (I) as described herein, or a pharmaceutically acceptable salt thereof, wherein R⁵ is halo-C₁-C₆-alkyl.

In a preferred embodiment, the present invention provides a compound of formula (I) as described herein, or a pharmaceutically acceptable salt thereof, wherein R⁵ is a group

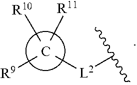

In a preferred embodiment, the present invention provides a compound of formula (I) as described herein, or a pharmaceutically acceptable salt thereof, wherein R⁵ is a group

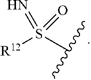

In a particularly preferred embodiment, the present invention provides a compound of formula (I) as described herein, or a pharmaceutically acceptable salt thereof, wherein R⁵ is selected from chloro, fluoro, cyano, CF₃, 2,2,2-trifluoroethyl, CF₃O, oxo, methylsulfonyl, CF₃-SO₂—, a group

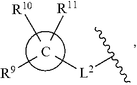

and a group

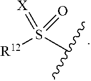

In a particularly preferred embodiment, the present invention provides a compound of formula (I) as described herein, or a pharmaceutically acceptable salt thereof, wherein R⁵ is CFA.

In a preferred embodiment, the present invention provides a compound of formula (I) as described herein, or a pharmaceutically acceptable salt thereof, wherein R⁶ is selected from hydrogen and oxo.

In a particularly preferred embodiment, the present invention provides a compound of formula (I) as described herein, or a pharmaceutically acceptable salt thereof, wherein R⁶ is hydrogen.

In a particularly preferred embodiment, the present invention provides a compound of formula (I) as described herein, or a pharmaceutically acceptable salt thereof, wherein R⁶ is oxo.

In a particularly preferred embodiment, the present invention provides a compound of formula (I) as described herein, or a pharmaceutically acceptable salt thereof, wherein R⁷ is hydrogen.

In a particularly preferred embodiment, the present invention provides a compound of formula (I) as described herein, or a pharmaceutically acceptable salt thereof, wherein R⁶ and R⁷ are both hydrogen.

In a particularly preferred embodiment, the present invention provides a compound of formula (I) as described herein, or a pharmaceutically acceptable salt thereof, wherein the group

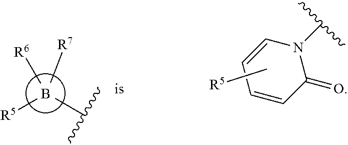

In one embodiment, the present invention provides a compound of formula (I) as described herein, or a pharmaceutically acceptable salt thereof, wherein:
A is selected from:

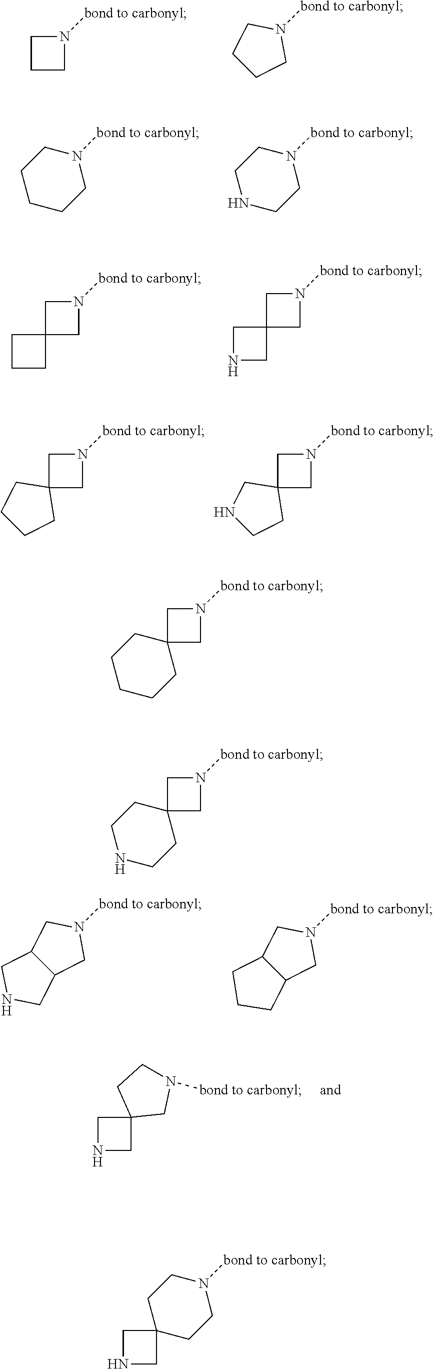

B is selected from C₆-C₁₄-aryl, C₃-C₁₀-cycloalkyl, 5- to 14-membered heteroaryl, and 3- to 14-membered heterocyclyl;
C is selected from C₆-C₁₄-aryl, C₃-C₁₀-cycloalkyl, 3- to 14-membered heterocyclyl, and 5- to 14-membered heteroaryl;
L¹ is selected from a covalent bond, —CHR⁸—, —CH₂O—, —OCH₂—, —CH₂NR^{13a}—, —NR^{13b}CH₂—, —CH₂CH₂—, —CH₂NHCH₂—, —CH₂NHCO—, —O—, —NH—, —SO₂NH—NHSO₂—, —SO₂NHCH₂—, —CH₂NHSO₂—, —SO₂—, —NHC(O)— and —C(O)NH—;
L² is selected from a covalent bond, —CH₂—, —NH—, —N(C₁-C₆-alkyl)-, —O—, —CH₂NH—, —NHCH₂—, —CH₂O—, and SO₂—;
R¹ is selected from a group

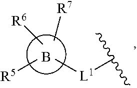

C₁-C₆-alkyl-SO₂—NH—, C₁-C₆-alkyl-SO₂—, and halo-C₁-C₆-alkyl-C(O)—;
R² is selected from hydrogen and hydroxy;
R³ is selected from hydrogen, halogen, cyano, C₁-C₆-alkyl, C₁-C₆-alkoxy, and hydroxy;
R⁴ is a group C(R^{4a}R^{4b}R^{4c});
R^{4a} is halo-C₁-C₆-alkyl; R^{4b} is hydroxy; and R^{4c} is hydrogen; or
R^{4a} and R^{4b}, taken together with the carbon atom to which they are attached, form a C₃-C₁₀-cycloalkyl; and R^{t} is hydrogen or hydroxy;
R⁵ is selected from hydrogen, hydroxy, halogen, C₁-C₆-alkyl, C₁-C₆-alkoxy, halo-C₁-C₆-alkyl, carboxy-C₁-C₆-alkyl, halo-C₁-C₆-alkoxy, oxo, NH₂—SO₂—, C₁-C₆-alkyl-SO₂—, halo-C₁-C₆-alkyl-SO₂—, C₁-C₆-alkyl-NH—C(O)—, carboxy, C₁-C₆-alkoxycarbonyl, C₁-C₆-alkoxycarbonyl-C₁-C₆-alkyl-, (C₁-C₆-alkyl)₂P(O)—C₁-C₆-alkyl-, a group

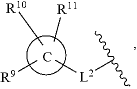

and a group

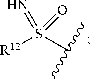

R⁶ is selected from hydrogen, halogen, cyano, oxo, C₁-C₆-alkoxy, halo-C₁-C₆-alkyl, and C₁-C₆-alkyl-SO₂—;
R⁷ is selected from hydrogen and C₁-C₆-alkyl;
R⁸ is selected from hydrogen and carbamoyl;
R⁹ is selected from hydrogen, halogen, hydroxy, oxo, carboxy, C₁-C₆-alkoxycarbonyl, carbamoyl, C₁-C₆-alkyl-NH—C(O)—, C₁-C₆-alkyl, hydroxy-C₁-C₆-alkyl, C₁-C₆-alkoxy-C₁-C₆-alkyl, C₃-C₁₀-cycloalkyl, cyano, C₁-C₆-alkyl-SO₂—, and 5- to 14-membered heteroaryl; wherein said C₃-C₁₀-cycloalkyl and 5- to 14-membered heteroaryl is optionally substituted with one substituent selected from C₁-C₆-alkyl and halo-C₁-C₆-alkyl;
R¹⁰ is selected from hydrogen, halogen, C₁-C₆-alkyl, halo-C₁-C₆-alkyl, and oxo;
R¹¹ is selected from hydrogen and halogen;
R¹² is selected from C₁-C₆-alkyl and halo-C₁-C₆-alkyl;
R^{13a} is selected from hydrogen, C₃-C₁₀-cycloalkyl, C₃-C₁₀-cycloalkyl-C₁-C₆-alkyl-; and
R^{13b} is hydrogen.

In one embodiment, the present invention provides a compound of formula (I) as described herein, or a pharmaceutically acceptable salt thereof, wherein A is selected from:

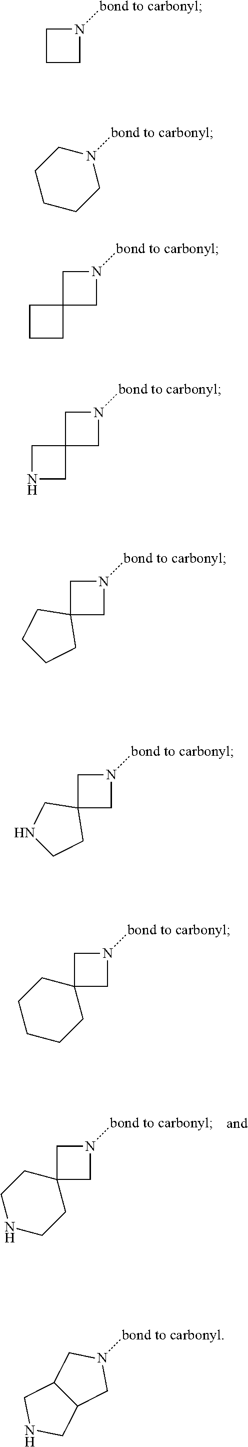

In a preferred embodiment, the present invention provides a compound of formula (I) as described herein, or a pharmaceutically acceptable salt thereof, wherein A is selected from:

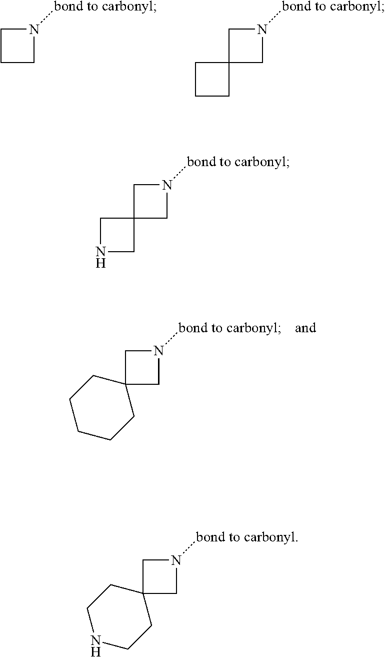

In a particularly preferred embodiment, the present invention provides a compound of formula (I) as described herein, or a pharmaceutically acceptable salt thereof, wherein A is

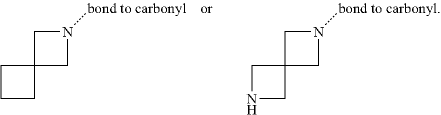

In a particularly preferred embodiment, the present invention provides a compound of formula (I) as described herein, or a pharmaceutically acceptable salt thereof, wherein A is

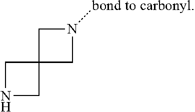

In a particularly preferred embodiment, the present invention provides a compound of formula (I) as described herein, or a pharmaceutically acceptable salt thereof, wherein A is

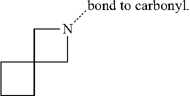

In one embodiment, the present invention provides a compound of formula (I) as described herein, or a pharmaceutically acceptable salt thereof, wherein B is selected from C₆-C₁₄-aryl, C₃-C₁₀-cycloalkyl, and 5- to 14-membered heteroaryl.

In a preferred embodiment, the present invention provides a compound of formula (I) as described herein, or a pharmaceutically acceptable salt thereof, wherein B is a 5- to 14-membered heteroaryl.

In a preferred embodiment, the present invention provides a compound of formula (I) as described herein, or a pharmaceutically acceptable salt thereof, wherein B is a 5- to 10-membered heteroaryl.

In a preferred embodiment, the present invention provides a compound of formula (I) as described herein, or a pharmaceutically acceptable salt thereof, wherein B is a 5- to 6-membered heteroaryl.

In a particularly preferred embodiment, the present invention provides a compound of formula (I) as described herein, or a pharmaceutically acceptable salt thereof, wherein B is selected from phenyl, bicyclo[1.1.1]pentane, pyridyl, oxazolyl, pyrimidine, pyrazine, pyridazine, 1,2,4-thiadiazole, pyrazolyl, imidazolyl, and 1H-1,2,4-triazole.

In a particularly preferred embodiment, the present invention provides a compound of formula (I) as described herein, or a pharmaceutically acceptable salt thereof, wherein B is phenyl.

In a particularly preferred embodiment, the present invention provides a compound of formula (I) as described herein, or a pharmaceutically acceptable salt thereof, wherein B is bicyclo[1.1.1]pentane.

In a particularly preferred embodiment, the present invention provides a compound of formula (I) as described herein, or a pharmaceutically acceptable salt thereof, wherein B is pyridyl.

In a particularly preferred embodiment, the present invention provides a compound of formula (I) as described herein, or a pharmaceutically acceptable salt thereof, wherein B is oxazolyl.

In a particularly preferred embodiment, the present invention provides a compound of formula (I) as described herein, or a pharmaceutically acceptable salt thereof, wherein B is pyrimidine.

In a particularly preferred embodiment, the present invention provides a compound of formula (I) as described herein, or a pharmaceutically acceptable salt thereof, wherein B is pyrazine.

In a particularly preferred embodiment, the present invention provides a compound of formula (I) as described herein, or a pharmaceutically acceptable salt thereof, wherein B is pyridazine.

In a particularly preferred embodiment, the present invention provides a compound of formula (I) as described herein, or a pharmaceutically acceptable salt thereof, wherein B is 1,2,4-thiadiazole.

In a particularly preferred embodiment, the present invention provides a compound of formula (I) as described herein, or a pharmaceutically acceptable salt thereof, wherein B is pyrazolyl.

In a particularly preferred embodiment, the present invention provides a compound of formula (I) as described herein, or a pharmaceutically acceptable salt thereof, wherein B is imidazolyl.

In a particularly preferred embodiment, the present invention provides a compound of formula (I) as described herein, or a pharmaceutically acceptable salt thereof, wherein B is 1H-1,2,4-triazole.

In one embodiment, the present invention provides a compound of formula (I) as described herein, or a pharmaceutically acceptable salt thereof, wherein Cis selected from C₃-C₁₀-cycloalkyl and 5- to 14-membered heteroaryl.

In a preferred embodiment, the present invention provides a compound of formula (I) as described herein, or a pharmaceutically acceptable salt thereof, wherein C is selected from cyclopropyl, pyrazolyl, and 1,3,4-oxadiazole.

In a particularly preferred embodiment, the present invention provides a compound of formula (I) as described herein, or a pharmaceutically acceptable salt thereof, wherein C is cyclopropyl.

In a particularly preferred embodiment, the present invention provides a compound of formula (I) as described herein, or a pharmaceutically acceptable salt thereof, wherein C is pyrazolyl.

In a particularly preferred embodiment, the present invention provides a compound of formula (I) as described herein, or a pharmaceutically acceptable salt thereof, wherein C is 1,3,4-oxadiazole.

In a preferred embodiment, the present invention provides a compound of formula (I) as described herein, or a pharmaceutically acceptable salt thereof, wherein:
L¹ is selected from a covalent bond, —CHR⁸—, —CH₂O—, and —SO₂—; and
R⁸ is hydrogen.

In a particularly preferred embodiment, the present invention provides a compound of formula (I) as described herein, or a pharmaceutically acceptable salt thereof, wherein L′ is a covalent bond.

In a particularly preferred embodiment, the present invention provides a compound of formula (I) as described herein, or a pharmaceutically acceptable salt thereof, wherein L¹ is —CH₂—.

In a particularly preferred embodiment, the present invention provides a compound of formula (I) as described herein, or a pharmaceutically acceptable salt thereof, wherein L¹ is —CH₂O—.

In a particularly preferred embodiment, the present invention provides a compound of formula (I) as described herein, or a pharmaceutically acceptable salt thereof, wherein L¹ is —SO₂—.

In a preferred embodiment, the present invention provides a compound of formula (I) as described herein, or a pharmaceutically acceptable salt thereof, wherein:
B is a 5- to 6-membered heteroaryl; and
L¹ is —CH₂—.

In a preferred embodiment, the present invention provides a compound of formula (I) as described herein, or a pharmaceutically acceptable salt thereof, wherein L² is selected from a covalent bond and —CH₂NH—.

In a preferred embodiment, the present invention provides a compound of formula (I) as described herein, or a pharmaceutically acceptable salt thereof, wherein R¹ is a group

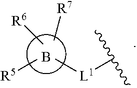

In a preferred embodiment, the present invention provides a compound of formula (I) as described herein, or a pharmaceutically acceptable salt thereof, wherein R² is hydrogen.

In one embodiment, the present invention provides a compound of formula (I) as described herein, or a pharmaceutically acceptable salt thereof, wherein R⁵ is selected from halogen, halo-C₁-C₆-alkyl, halo-C₁-C₆-alkoxy, C₁-C₆-alkyl-SO₂—, halo-C₁-C₆-alkyl-SO₂—, a group

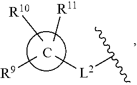

and a group

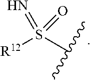

In a preferred embodiment, the present invention provides a compound of formula (I) as described herein, or a pharmaceutically acceptable salt thereof, wherein R⁵ is selected from chloro, fluoro, CF₃, CF₃O, methylsulfonyl, CF₃—SO₂—, a group

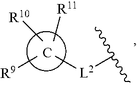

and a group

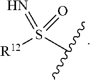

In a preferred embodiment, the present invention provides a compound of formula (I) as described herein, or a pharmaceutically acceptable salt thereof, wherein R⁵ is halo-C₁-C₆-alkyl.

In a particularly preferred embodiment, the present invention provides a compound of formula (I) as described herein, or a pharmaceutically acceptable salt thereof, wherein R⁵ is CF₃.

In a preferred embodiment, the present invention provides a compound of formula (I) as described herein, or a pharmaceutically acceptable salt thereof, wherein:
B is a 5- to 6-membered heteroaryl;
L¹ is —CH₂—; and
R⁵ is halo-C₁-C₆-alkyl.

In a preferred embodiment, the present invention provides a compound of formula (I) as described herein, or a pharmaceutically acceptable salt thereof, wherein R⁶ is selected from hydrogen, halogen, and cyano.

In a particularly preferred embodiment, the present invention provides a compound of formula (I) as described herein, or a pharmaceutically acceptable salt thereof, wherein R⁶ is selected from hydrogen, fluoro, and cyano.

In a particularly preferred embodiment, the present invention provides a compound of formula (I) as described herein, or a pharmaceutically acceptable salt thereof, wherein R⁶ is hydrogen.

In a particularly preferred embodiment, the present invention provides a compound of formula (I) as described herein, or a pharmaceutically acceptable salt thereof, wherein:
B is a 5- to 6-membered heteroaryl;
L¹ is —CH₂—;
R⁵ is halo-C₁-C₆-alkyl; and
R⁶ is hydrogen.

In a preferred embodiment, the present invention provides a compound of formula (I) as described herein, or a pharmaceutically acceptable salt thereof, wherein R⁷ is hydrogen.

In a particularly preferred embodiment, the present invention provides a compound of formula (I) as described herein, or a pharmaceutically acceptable salt thereof, wherein:
B is a 5- to 6-membered heteroaryl;
L¹ is —CH₂;
R⁵ is halo-C₁-C₆-alkyl;
R⁶ is hydrogen; and
R⁷ is hydrogen

In a preferred embodiment, the present invention provides a compound of formula (I) as described herein, or a pharmaceutically acceptable salt thereof, wherein R⁸ is hydrogen.

In a preferred embodiment, the present invention provides a compound of formula (I) as described herein, or a pharmaceutically acceptable salt thereof, wherein R⁹ is selected from C₁-C₆-alkyl and halo-C ₁-C₆-alkyl.

In a particularly preferred embodiment, the present invention provides a compound of formula (I) as described herein, or a pharmaceutically acceptable salt thereof, wherein R⁹ is selected from methyl, 2,2-dimethylpropyl, and CF₃.

In a preferred embodiment, the present invention provides a compound of formula (I) as described herein, or a pharmaceutically acceptable salt thereof, wherein R¹⁰ is selected from hydrogen and halo-C₁-C₆-alkyl.

In a particularly preferred embodiment, the present invention provides a compound of formula (I) as described herein, or a pharmaceutically acceptable salt thereof, wherein R¹⁰ is selected from hydrogen and CF₃.

In a preferred embodiment, the present invention provides a compound of formula (I) as described herein, or a pharmaceutically acceptable salt thereof, wherein R¹¹ is hydrogen.

In a preferred embodiment, the present invention provides a compound of formula (I) as described herein, or a pharmaceutically acceptable salt thereof, wherein:
C is selected from C₃-C₁₀-cycloalkyl and 5- to 14-membered heteroaryl;
L² is selected from a covalent bond and —CH₂NH—;
R⁹ is selected from C₁-C₆-alkyl and halo-C₁-C₆-alkyl;
R¹⁰ is selected from hydrogen and halo-C₁-C₆-alkyl; and
R¹¹ is hydrogen.

In a particularly preferred embodiment, the present invention provides a compound of formula (I) as described herein, or a pharmaceutically acceptable salt thereof, wherein:
C is selected from cyclopropyl, pyrazolyl, and 1,3,4-oxadiazole;
L² is selected from a covalent bond and —CH₂NH—;
R⁹ is selected from C₁-C₆-alkyl and halo-C₁-C₆-alkyl;
R¹⁰ is selected from hydrogen and halo-C₁-C₆-alkyl; and
R¹¹ is hydrogen.

In a preferred embodiment, the present invention provides a compound of formula (I) as described herein, or a pharmaceutically acceptable salt thereof, wherein R¹² is halo-C₁-C₆-alkyl.

In a particularly preferred embodiment, the present invention provides a compound of formula (I) as described herein, or a pharmaceutically acceptable salt thereof, wherein R¹² is CF₃.

In a preferred embodiment, the present invention provides a compound of formula (I) as described herein, or a pharmaceutically acceptable salt thereof, wherein:
B is selected from C₆-C₁₄-aryl, C₃-C₁₀-cycloalkyl, and 5- to 14-membered heteroaryl;
C is selected from C₃-C₁₀-cycloalkyl and 5- to 14-membered heteroaryl;
L¹ is selected from a covalent bond, —CHR⁸, —CH₂O—, and —SO₂—;
L² is selected from a covalent bond and —CH₂NH—;
R¹ is a group

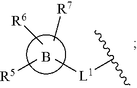

R² is hydrogen;
R⁵ is selected from halogen, halo-C₁-C₆-alkyl, halo-C₁-C₆-alkoxy, C₁-C₆-alkyl-SO₂—, halo-C₁-C₆-alkyl-SO₂—, a group

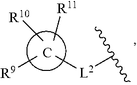

and a group

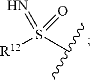

R⁶ is selected from hydrogen, halogen, and cyano;
R⁷ is hydrogen; R⁸ is hydrogen;
R⁹ is selected from C₁-C₆-alkyl and halo-C₁-C₆-alkyl;
R¹⁰ is selected from hydrogen and halo-C₁-C₆-alkyl;
R¹¹ is hydrogen; and
R¹² is halo-C₁-C₆-alkyl.

In a particularly preferred embodiment, the present invention provides a compound of formula (I) as described herein, or a pharmaceutically acceptable salt thereof, wherein:
B is selected from phenyl, bicyclo[1.1.1]pentane, pyridyl, oxazolyl, pyrimidine, pyrazine, pyridazine, 1,2,4-thiadiazole, pyrazolyl, imidazolyl, and 1H-1,2,4-triazole;
C is selected from cyclopropyl, pyrazolyl, and 1,3,4-oxadiazole;
L¹ is selected from a covalent bond, —CR⁸R⁹—, —CH₂O—, and —SO₂—;
L² is selected from a covalent bond and —CH₂NH—;
R¹ is a group

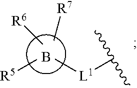

R² is hydrogen;
R⁵ is selected from chloro, fluoro, CF₃, CF₃O, methylsulfonyl, CF₃-SO₂—, a group

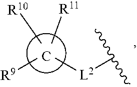

and a group

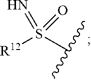

R⁶ is selected from hydrogen, fluoro, and cyano;
R⁷ is hydrogen;
R⁸ is hydrogen;
R⁹ is selected from methyl, 2,2-dimethylpropyl, and CF₃;
R¹⁰ is selected from hydrogen and CF₃;
R¹¹ is hydrogen; and
R¹² is CF₃.

In a preferred embodiment, the present invention provides a compound of formula (I) as described herein, or a pharmaceutically acceptable salt thereof, wherein R³ is hydrogen.

In a preferred embodiment, the present invention provides a compound of formula (I) as described herein, or a pharmaceutically acceptable salt thereof, wherein:
R⁴ is a group —C(R^{4a}R^{4b}R^{4c}); wherein
R^{4a} and R^{4b}, taken together with the carbon atom to which they are attached, form a C₃-C₁₀-cycloalkyl; and
R^{4c} is hydrogen or hydroxy.

In a particularly preferred embodiment, the present invention provides a compound of formula (I) as described herein, or a pharmaceutically acceptable salt thereof, wherein:
R⁴ is a group —C(R^{4a}R^{4b}R^{4c}); wherein
R^{4a} and R^{4b}, taken together with the carbon atom to which they are attached, form a cyclopropyl; and
R^{4c} is hydrogen or hydroxy.

In a particularly preferred embodiment, the present invention provides a compound of formula (I) as described herein, or a pharmaceutically acceptable salt thereof, wherein R⁴ is C₃-C₁₀-cycloalkyl.

In a particularly preferred embodiment, the present invention provides a compound of formula (I) as described herein, or a pharmaceutically acceptable salt thereof, wherein R⁴ is cyclopropyl.

In one embodiment, the present invention provides a compound of formula (I) as described herein, or a pharmaceutically acceptable salt thereof, wherein:
A is selected from:

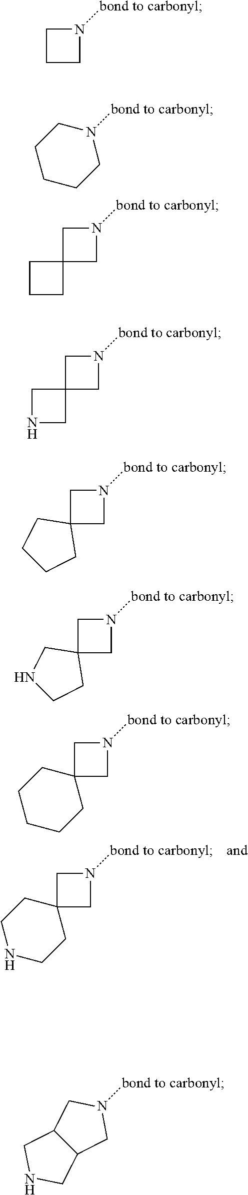

B is selected from C₆-C₁₄-aryl, C₃-C₁₀-cycloalkyl, 5- to 14-membered heteroaryl, and 3- to 14-membered heterocyclyl;
C is selected from C₆-C₁₄-aryl, C₃-C₁₀-cycloalkyl, 3- to 14-membered heterocyclyl, and 5- to 14-membered heteroaryl;
L¹ is selected from a covalent bond, —CHR⁸—, —CH₂O—, —OCH₂—, —CH₂NR^{13a}—, —NR^{13b}CH₂, —CH₂CH₂—, —CH₂NHCH₂—, —CH₂NHCO—, —O—, —NH—, —SO₂NH—, —NHSO₂—, —SO₂NHCH₂—, —CH₂NHSO₂—, —SO₂—, —NHC(O)— and —C(O)NH—;
L² is selected from a covalent bond, —CH₂—, —NH—, —N(C₁-C₆-alkyl)-, —O—, —CH₂NH—, —NHCH₂—, —CH₂O—, and SO₂—;
R¹ is selected from a group

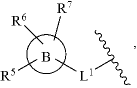

C₁-C₆-alkyl-SO₂—NH—, C₁-C₆-alkyl-SO₂—, and halo-C₁-C₆-alkyl-C(O)—;
R² is selected from hydrogen and hydroxy;
R³ is hydrogen;
R⁴ is a group
R^{4a} is halo-C₁-C₆-alkyl; R^{4b} is hydroxy; and R^{4c} is hydrogen; or
R^{4a} and R^{4b}, taken together with the carbon atom to which they are attached, form a C₃-C₁₀-cycloalkyl; and R is hydrogen or hydroxy;
R⁵ is selected from hydrogen, hydroxy, halogen, C₁-C₆-alkyl, C₁-C₆-alkoxy, halo-C₁-C₆-alkyl, carboxy-C₁-C₆-alkyl, halo-C₁-C₆-alkoxy, oxo, NH₂—SO₂—, C₁-C₆-alkyl-SO₂—, halo-C₁-C₆-alkyl-SO₂—, C₁-C₆-alkyl-NH—C(O)—, carboxy, C₁-C₆-alkoxycarbonyl, C₁-C₆-alkoxycarbonyl-C₁-C₆-alkyl-, (C₁-C₆-alkyl)₂P(O)—C₁-C₆-alkyl-, a group

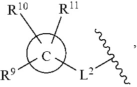

and a group

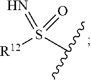

R⁶ is selected from hydrogen, halogen, cyano, oxo, C₁-C₆-alkoxy, halo-C₁-C₆-alkyl, and C₁-C₆-alkyl-SO₂—;
R⁷ is selected from hydrogen, C₁-C₆-alkyl;
R⁸ is selected from hydrogen and carbamoyl;
R⁹ is selected from hydrogen, halogen, hydroxy, oxo, carboxy, C₁-C₆ alkoxycarbonyl, carbamoyl, C₁-C₆-alkyl-NH—C(O)—, C₁-C₆-alkyl, hydroxy-C₁—CF-alkyl, halo-C₁-C₆-alkyl, C₁-C₆-alkoxy-C₁-C₆-alkyl, C₃-C₁₀-cycloalkyl, cyano, C₁-C₆-alkyl-SO₂—, and 5- to 14-membered heteroaryl; wherein said C₃-C₁₀-cycloalkyl and 5- to 14-membered heteroaryl is optionally substituted with one substituent selected from C₁-C₆-alkyl and halo-C₁-C₆-alkyl;
R¹⁰ is selected from hydrogen, halogen, C₁-C₆-alkyl, halo-C₁-C₆-alkyl, and oxo;
R¹¹ is selected from hydrogen and halogen;
R¹² is selected from C₁-C₆-alkyl and halo-C₁-C₆-alkyl;
R^{13a} is selected from hydrogen, C₃-C₁₀-cycloalkyl, C₃-C₁₀-cycloalkyl-C₁-C₆-alkyl-; and
R^{13b} is hydrogen.

In a preferred embodiment, the present invention provides a compound of formula (I) as described herein, or a pharmaceutically acceptable salt thereof, wherein:
A is selected from:

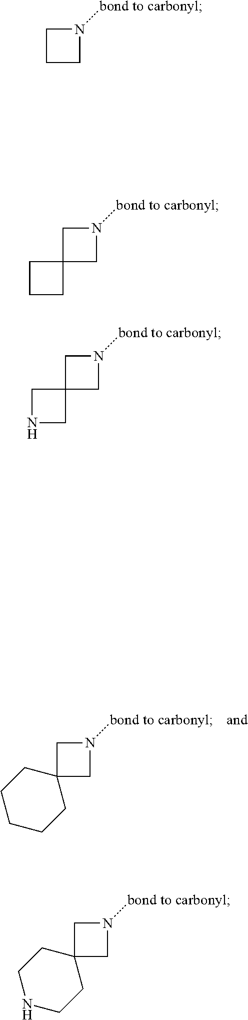

B is selected from C₆-C₁₄-aryl, C₃-C₁₀-cycloalkyl, and 5- to 14-membered heteroaryl;
C is selected from C₃-C₁₀-cycloalkyl and 5- to 14-membered heteroaryl;
L¹ is selected from a covalent bond, —CHR⁸—, —CH₂O—, and —SO₂—,
L² is selected from a covalent bond and —CH₂NH—;
R¹ is a group

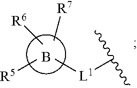

R² is hydrogen;
R³ is hydrogen;
R⁴ is a group —C(R^{4a}R^{4b}R^{4c});
R^{4a} and R^{4b}, taken together with the carbon atom to which they are attached, form a C₃-C₁₀-cycloalkyl;
R^{4c} is hydrogen or hydroxy;
R⁵ is selected from halogen, halo-C₁-C₆-alkyl, halo-C₁-C₆-alkoxy, C₁-C₆-alkyl-SO₂—, halo-C₁-C₆-alkyl-SO₂—, a group

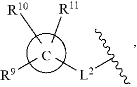

and a group

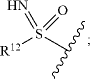

R⁶ is selected from hydrogen, halogen, and cyano;
R⁷ is hydrogen;
R⁸ is selected from hydrogen, halogen, and cyan;
R⁹ is selected from C₁-C₆-alkyl and halo-C₁-C₆-alkyl;
R¹⁰ is selected from hydrogen and halo-C₁-C₆-alkyl;
R¹¹ is hydrogen; and
R¹² is halo-C₁-C₆-alkyl.

In a particularly preferred embodiment, the present invention provides a compound of formula (I) as described herein, or a pharmaceutically acceptable salt thereof, wherein:
A is selected from:

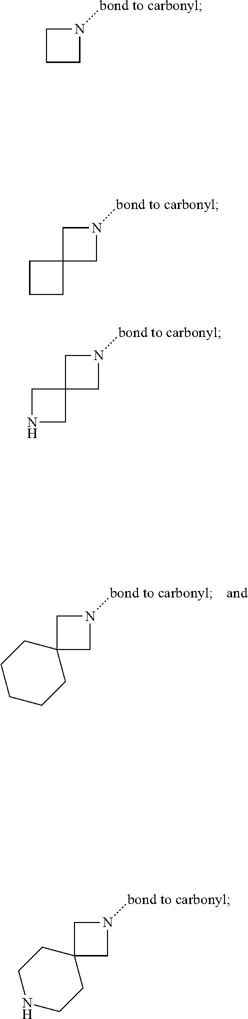

B is selected from phenyl, bicyclo[1.1.1]pentane, pyridyl, oxazolyl, pyrimidine, pyrazine, pyridazine, 1,2,4-thiadiazole, pyrazolyl, imidazolyl, and 1H-1,2,4-triazole;
C is selected from cyclopropyl, pyrazolyl, and 1,3,4-oxadiazole;
L¹ is selected from a covalent bond, —CR⁸R⁹—, —CH₂O—, and —SO₂—;
L² is selected from a covalent bond and —CH₂NH—;
R¹ is a group

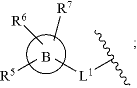

R² is hydrogen;
R³ is hydrogen;
R⁴ is a group —C(R^{4a}R^{4b}R^{4c});
R^{4a} and R^{4b}, taken together with the carbon atom to which they are attached, form a cyclopropyl;
R^{4c} is hydrogen or hydroxy;
R⁵ is selected from chloro, fluoro, CF₃, CF₃O, methylsulfonyl, CF₃-SO₂—, a group

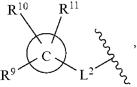

and a group

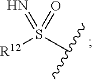

R⁶ is selected from hydrogen, fluoro, and cyano;
R⁷ is hydrogen;
R⁸ is hydrogen;
R⁹ is selected from methyl, 2,2-dimethylpropyl, and CF₃;
R¹⁰ is selected from hydrogen and CF₃;
R¹¹ is hydrogen; and
R¹² is CF₃.

In one embodiment, the present invention provides a compound of formula (I) as described herein, or a pharmaceutically acceptable salt thereof, wherein said compound of formula (I) is selected from:
[6-(5-cyclopropyl-4H-1,2,4-triazol-3-yl)-2-azaspiro[3.3]heptan-2-yl]-[3-[6-[[1-(trifluoromethyl)cyclopropyl]amino]-3-pyridyl]azetidin-1-yl]methanone;
[6-(3-cyclopropyl-1H-1,2,4-triazol-5-yl)-2-azaspiro[3.3]heptan-2-yl]-[6-[4-(trifluoromethylsulfonimidoyl)benzyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[5-(trifluoromethyl)pyrazin-2-yl]methyl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[[5-(trifluoromethyl)pyrazin-2-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[6-(trifluoromethyl)-3-pyridyl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[6-(trifluoromethyl)-3-pyridyl]methyl]-2-azaspiro[3.3]heptan-2-yl]methanone;
[6-(5-cyclobutyl-4H-1,2,4-triazol-3-yl)-2-azaspiro[3.3]heptan-2-yl]-[6-[[6-(trifluoromethyl)-3-pyridyl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[5-(trifluoromethoxy)-2-pyridyl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[5-(trifluoromethoxy)-2-pyridyl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[3-fluoro-5-(trifluoromethyl)-2-pyridyl]methyl]-2-azaspiro[3.3]heptan-2-yl]methanone;
[6-[[3-fluoro-5-(trifluoromethyl)-2-pyridyl]methyl]-2-azaspiro[3.3]heptan-2-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[6-[(4-methylsulfonylphenyl)methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[(4-methylsulfonylphenyl)methyl]-2-azaspiro[3.3]heptan-2-yl]methanone;
[6-(5-cyclobutyl-4H-1,2,4-triazol-3-yl)-2-azaspiro[3.3]heptan-2-yl]-[6-[(4-methylsulfonylphenyl)methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[(3-methylsulfonylphenyl)methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[(3-methylsulfonylphenyl)methyl]-2-azaspiro[3.3]heptan-2-yl]methanone;
[6-(5-cyclobutyl-4H-1,2,4-triazol-3-yl)-2-azaspiro[3.3]heptan-2-yl]-[6-[(3-methylsulfonylphenyl)methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[(3-fluoro-5-methylsulfonyl-phenyl)methyl]-2-azaspiro[3.3]heptan-2-yl]methanone;
[6-[(3-fluoro-5-methylsulfonyl-phenyl)methyl]-2-azaspiro[3.3]heptan-2-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[6-[[2-(trifluoromethyl)pyrimidin-5-yl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[2-(trifluoromethyl)pyrimidin-5-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[6-(trifluoromethyl)pyridazin-3-yl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[6-(trifluoromethyl)pyridazin-3-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[(4-fluoro-2-methylsulfonyl-phenyl)methyl]-2-azaspiro[3.3]heptan-2-yl]methanone;
[6-(5-cyclobutyl-4H-1,2,4-triazol-3-yl)-2-azaspiro[3.3]heptan-2-yl]-[6-[(4-fluoro-2-methylsulfonyl-phenyl)methyl]-2-azaspiro[3.3]heptan-2-yl]methanone;
5-[[2-[6-(5-cyclopropyl-4H-1,2,4-triazol-3-yl)-2-azaspiro[3.3]heptane-2-carbonyl]-2-azaspiro[3.3]heptan-6-yl]methyl]-2-(trifluoromethyl)benzonitrile;
5-[[2-[6-[5-(1-hydroxycyclopropyl)-4H-1,2,4-triazol-3-yl]-2-azaspiro[3.3]heptane-2-carbonyl]-2-azaspiro[3.3]heptan-6-yl]methyl]-2-(trifluoromethyl)benzonitrile;
[6-(5-cyclopropyl-4H-1,2,4-triazol-3-yl)-2-azaspiro[3.3]heptan-2-yl]-[6-[[5-(trifluoromethyl)pyrimidin-2-yl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[5-(trifluoromethyl)pyrimidin-2-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[3-(trifluoromethylsulfonyl)phenyl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[3-(trifluoromethylsulfonyl)phenyl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[4-fluoro-2-(trifluoromethyl)phenyl]methyl]-2-azaspiro[3.3]heptan-2-yl]methanone;
[6-[[4-fluoro-2-(trifluoromethyl)phenyl]methyl]-2-azaspiro[3.3]heptan-2-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[6-[(2,4-difluorophenyl)methyl]-2-azaspiro[3.3]heptan-2-yl]methanone;
[6-[(2,4-difluorophenyl)methyl]-2-azaspiro[3.3]heptan-2-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[6-[[2-fluoro-4-(trifluoromethyl)phenyl]methyl]-2-azaspiro[3.3]heptan-2-yl]methanone;
[6-[[2-fluoro-4-(trifluoromethyl)phenyl]methyl]-2-azaspiro[3.3]heptan-2-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[6-[[2-methylsulfonyl-4-(trifluoromethyl)phenyl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[2-methylsulfonyl-4-(trifluoromethyl)phenyl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[3-methylsulfonyl-4-(trifluoromethyl)phenyl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[3-methylsulfonyl-4-(trifluoromethyl)phenyl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[(5-fluoro-2-pyridyl)methyl]-2-azaspiro[3.3]heptan-2-yl]methanone;
[6-[(5-fluoro-2-pyridyl)methyl]-2-azaspiro[3.3]heptan-2-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[6-[(5-fluoro-3-pyridyl)methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[4-methylsulfonyl-3-trifluoromethyl)phenyl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[4-methylsulfonyl-3-(trifluoromethyl)phenyl]methyl]-2-azaspiro[3.3]heptan-2-yl]methanone;
[6-(5-cyclopropyl-4H-1,2,4-triazol-3-yl)-2-azaspiro[3.3]heptan-2-yl]-[7-[[5-(trifluoromethyl)-2-pyridyl]methyl]-2-azaspiro[3.5]nonan-2-yl]methanone;
[6-[5-(1-hydroxycyclopropyl)-4H-1,2,4-triazol-3-yl]-2-azaspiro[3.3]heptan-2-yl]-[7-[[5-(trifluoromethyl)-2-pyridyl]methyl]-2-azaspiro[3.5]nonan-2-yl]methanone;
[6-(5-cyclopropyl-4H-1,2,4-triazol-3-yl)-2-azaspiro[3.3]heptan-2-yl]-[6-[[5-(trifluoromethyl)-2-pyridyl]methyl]-2-azaspiro[3.4]octan-2-yl]methanone;
[6-(5-cyclopropyl-4H-1,2,4-triazol-3-yl)-2-azaspiro[3.3]heptan-2-yl]-[7-[6-(trifluoromethyl)pyridazin-3-yl]oxy-2-azaspiro[3.5]nonan-2-yl]methanone;
[6-(5-cyclobutyl-4H-1,2,4-triazol-3-yl)-2-azaspiro[3.3]heptan-2-yl]-[7-[6-(trifluoromethyl)pyridazin-3-yl]oxy-2-azaspiro[3.5]nonan-2-yl]methanone;
[6-(5-cyclopropyl-4H-1,2,4-triazol-3-yl)-2-azaspiro[3.3]heptan-2-yl]-[3-[6-(4-methyl-4-oxo-1,4λ⁵-azaphosphinan-1-yl)-3-pyridyl]azetidin-1-yl]methanone;
[6-(5-cyclopropyl-4H-1,2,4-triazol-3-yl)-2-azaspiro[3.3]heptan-2-yl]-[6-[[[1-(trifluoromethyl)cyclopropyl]amino]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[[1-(trifluoromethyl)cyclopropyl]amino]methyl]-2-azaspiro[3.3]heptan-2-yl]methanone;
N-[2-[6-(5-cyclopropyl-4H-1,2,4-triazol-3-yl)-2-azaspiro[3.3]heptane-2-carbonyl]-2-azaspiro[3.3]heptan-6-yl]-3-(trifluoromethyl)benzenesulfonamide;
[6-(5-cyclopropyl-4H-1,2,4-triazol-3-yl)-2-azaspiro[3.3]heptan-2-yl]-[6-[[6-(trifluoromethyl)pyridazin-3-yl]amino]-2-azaspiro[3.3]heptan-2-yl]methanone;
[6-(5-cyclopropyl-4H-1,2,4-triazol-3-yl)-2-azaspiro[3.3]heptan-2-yl]-[6-[[4-(trifluoromethyl)pyrazol-1-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[3-(trifluoromethyl)-1,2,4-thiadiazol-5-yl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[3-(trifluoromethyl)-1,2,4-thiadiazol-5-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[4-(trifluoromethyl)oxazol-2-yl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[4-(trifluoromethyl)oxazol-2-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[4-(trifluoromethyl)imidazol-1-yl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[4-(trifluoromethyl)imidazol-1-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[7-[[1-(trifluoromethyl)cyclopropyl]methoxy]-2-azaspiro[3.5]nonan-2-yl]methanone;
[6-[5-(1-hydroxycyclopropyl)-4H-1,2,4-triazol-3-yl]-2-azaspiro[3.3]heptan-2-yl]-[7-[[1-(trifluoromethyl)cyclopropyl]methoxy]-2-azaspiro[3.5]nonan-2-yl]methanone;
[6-(5-cyclopropyl-4H-1,2,4-triazol-3-yl)-2-azaspiro[3.3]heptan-2-yl]-[6-[5-(trifluoromethyl)pyrazin-2-yl]-2-azaspiro[3.3]heptan-2-yl]methanone;
[6-(5-cyclopropyl-4H-1,2,4-triazol-3-yl)-2-azaspiro[3.3]heptan-2-yl]-[6-[[1-(trifluoromethyl)cyclopropyl]methoxy]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[1-(trifluoromethyl)cyclopropyl]methoxy]-2-azaspiro[3.3]heptan-2-yl]methanone;
[6-(5-cyclopropyl-4H-1,2,4-triazol-3-yl)-2-azaspiro[3.3]heptan-2-yl]-[6-[[1-(trifluoromethyl)cyclopropyl]amino]-2-azaspiro[3.3]heptan-2-yl]methanone;
[6-(5-cyclopropyl-4H-1,2,4-triazol-3-yl)-2-azaspiro[3.3]heptan-2-yl]-[6-[[3-(trifluoromethyl)-1,2,4-triazol-1-yl]methyl]-2-azaspiro[3.3]heptan-2-yl]methanone;
2-[6-(5-cyclopropyl-4H-1,2,4-triazol-3-yl)-2-azaspiro[3.3]heptane-2-carbonyl]-N-[1-(trifluoromethyl)cyclopropyl]-2-azaspiro[3.3]heptane-6-carboxamide;
[6-(5-cyclopropyl-4H-1,2,4-triazol-3-yl)-2-azaspiro[3.3]heptan-2-yl]-[7-[2-(trifluoromethyl)pyrimidin-4-yl]oxy-2-azaspiro[3.5]nonan-2-yl]methanone;
[6-(5-cyclopropyl-4H-1,2,4-triazol-3-yl)-2-azaspiro[3.3]heptan-2-yl]-[7-[5-(trifluoromethyl)pyrazin-2-yl]oxy-2-azaspiro[3.5]nonan-2-yl]methanone;
[6-(5-cyclopropyl-4H-1,2,4-triazol-3-yl)-2-azaspiro[3.3]heptan-2-yl]-[6-[5-(trifluoromethyl)pyrazin-2-yl]oxy-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[5-(trifluoromethyl)pyrazin-2-yl]oxy-2-azaspiro[3.3]heptan-2-yl]methanone;
[6-(5-cyclopropyl-4H-1,2,4-triazol-3-yl)-2-azaspiro[3.3]heptan-2-yl]-[6-[(4-methylsulfonylphenyl)methyl]-2-azaspiro[3.4]octan-2-yl]methanone;
[6-(5-cyclopropyl-4H-1,2,4-triazol-3-yl)-2-azaspiro[3.3]heptan-2-yl]-[6-[6-(trifluoromethyl)pyridazin-3-yl]oxy-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[6-(trifluoromethyl)pyridazin-3-yl]oxy-2-azaspiro[3.3]heptan-2-yl]methanone;
[6-(5-cyclopropyl-4H-1,2,4-triazol-3-yl)-2-azaspiro[3.3]heptan-2-yl]-[3-[6-(1,1-dioxo-1,4-thiazinan-4-yl)-3-pyridyl]azetidin-1-yl]methanone;
[6-(5-cyclopropyl-4H-1,2,4-triazol-3-yl)-2-azaspiro[3.3]heptan-2-yl]-[3-[6-[(1,1-dioxothiolan-3-yl)amino]-3-pyridyl]azetidin-1-yl]methanone;
[6-(5-cyclopropyl-4H-1,2,4-triazol-3-yl)-2-azaspiro[3.3]heptan-2-yl]-[3-[6-(3-hydroxy-3-methyl-azetidin-1-yl)-3-pyridyl]azetidin-1-yl]methanone;
[6-(5-cyclopropyl-4H-1,2,4-triazol-3-yl)-2-azaspiro[3.3]heptan-2-yl]-[6-[3-(trifluoromethyl)azetidin-1-yl]-2-azaspiro[3.3]heptan-2-yl]methanone;
[6-(5-cyclopropyl-4H-1,2,4-triazol-3-yl)-2-azaspiro[3.3]heptan-2-yl]-[6-[3-(2,2,2-trifluoroethyl)azetidin-1-yl]-2-azaspiro[3.3]heptan-2-yl]methanone;
[6-(5-cyclopropyl-4H-1,2,4-triazol-3-yl)-2-azaspiro[3.3]heptan-2-yl]-[7-[[4-(trifluoromethylsulfonyl)phenyl]methyl]-2,7-diazaspiro[3.5]nonan-2-yl]methanone.
[6-(5-cyclopropyl-4H-1,2,4-triazol-3-yl)-2-azaspiro[3.3]heptan-2-yl]-[6-[[4-(trifluoromethylsulfonyl)phenyl]methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[2-[3-(trifluoromethoxy)phenyl]sulfonyl-2,6-diazaspiro[3.3]heptan-6-yl]methanone;
[6-[5-(1-hydroxycyclopropyl)-4H-1,2,4-triazol-3-yl]-2-azaspiro[3.3]heptan-2-yl]-[2-[3-(trifluoromethoxy)phenyl]sulfonyl-2,6-diazaspiro[3.3]heptan-6-yl]methanone;
[6-(5-cyclobutyl-4H-1,2,4-triazol-3-yl)-2-azaspiro[3.3]heptan-2-yl]-[2-[3-(trifluoromethoxy)phenyl]sulfonyl-2,6-diazaspiro[3.3]heptan-6-yl]methanone;
[6-(5-cyclopropyl-4H-1,2,4-triazol-3-yl)-2-azaspiro[3.3]heptan-2-yl]-[(6S)-6-[5-(trifluoromethyl)pyrazin-2-yl]oxy-2-azaspiro[3.4]octan-2-yl]methanone;
[6-(5-cyclopropyl-4H-1,2,4-triazol-3-yl)-2-azaspiro[3.3]heptan-2-yl]-[2-[4-(trifluoromethoxy)phenyl]sulfonyl-2,6-diazaspiro[3.3]heptan-6-yl]methanone;
[6-[5-(1-hydroxycyclopropyl)-4H-1,2,4-triazol-3-yl]-2-azaspiro[3.3]heptan-2-yl]-[2-[4-(trifluoromethoxy)phenyl]sulfonyl-2,6-diazaspiro[3.3]heptan-6-yl]methanone;
[6-(5-cyclobutyl-4H-1,2,4-triazol-3-yl)-2-azaspiro[3.3]heptan-2-yl]-[2-[4-(trifluoromethoxy)phenyl]sulfonyl-2,6-diazaspiro[3.3]heptan-6-yl]methanone;
[6-(5-cyclopropyl-4H-1,2,4-triazol-3-yl)-2-azaspiro[3.3]heptan-2-yl]-[2-[2-(trifluoromethoxy)phenyl]sulfonyl-2,6-diazaspiro[3.3]heptan-6-yl]methanone;
[6-[5-(1-hydroxycyclopropyl)-4H-1,2,4-triazol-3-yl]-2-azaspiro[3.3]heptan-2-yl]-[2-[2-(trifluoromethoxy)phenyl]sulfonyl-2,6-diazaspiro[3.3]heptan-6-yl]methanone;
[6-(5-cyclopropyl-4H-1,2,4-triazol-3-yl)-2-azaspiro[3.3]heptan-2-yl]-[2-[2-(trifluoromethyl)phenyl]sulfonyl-2,6-diazaspiro[3.3]heptan-6-yl]methanone;
[6-[5-(1-hydroxycyclopropyl)-4H-1,2,4-triazol-3-yl]-2-azaspiro[3.3]heptan-2-yl]-[2-[2-(trifluoromethyl)phenyl]sulfonyl-2,6-diazaspiro[3.3]heptan-6-yl]methanone;
[6-(5-cyclopropyl-4H-1,2,4-triazol-3-yl)-2-azaspiro[3.3]heptan-2-yl]-[2-[4-fluoro-2-(trifluoromethyl)phenyl]sulfonyl-2,6-diazaspiro[3.3]heptan-6-yl]methanone;
[6-(5-cyclopropyl-4H-1,2,4-triazol-3-yl)-2-azaspiro[3.3]heptan-2-yl]-[2-[3-(trifluoromethyl)phenyl]sulfonyl-2,6-diazaspiro[3.3]heptan-6-yl]methanone;
[6-[5-(1-hydroxycyclopropyl)-4H-1,2,4-triazol-3-yl]-2-azaspiro[3.3]heptan-2-yl]-[2-[3-(trifluoromethyl)phenyl]sulfonyl-2,6-diazaspiro[3.3]heptan-6-yl]methanone;
[6-(5-cyclopropyl-4H-1,2,4-triazol-3-yl)-2-azaspiro[3.3]heptan-2-yl]-[2-[3-fluoro-5-(trifluoromethyl)phenyl]sulfonyl-2,6-diazaspiro[3.3]heptan-6-yl]methanone;
[6-(5-cyclopropyl-4H-1,2,4-triazol-3-yl)-2-azaspiro[3.3]heptan-2-yl]-[2-[4-fluoro-3-(trifluoromethyl)phenyl]sulfonyl-2,6-diazaspiro[3.3]heptan-6-yl]methanone;
[6-(5-cyclopropyl-4H-1,2,4-triazol-3-yl)-2-azaspiro[3.3]heptan-2-yl]-[2-[4-(trifluoromethyl)phenyl]sulfonyl-2,6-diazaspiro[3.3]heptan-6-yl]methanone;
[6-[5-(1-hydroxycyclopropyl)-4H-1,2,4-triazol-3-yl]-2-azaspiro[3.3]heptan-2-yl]-[2-[4-(trifluoromethyl)phenyl]sulfonyl-2,6-diazaspiro[3.3]heptan-6-yl]methanone;
[6-(5-cyclopropyl-4H-1,2,4-triazol-3-yl)-2-azaspiro[3.3]heptan-2-yl]-[2-(2,4-difluorophenyl)sulfonyl-2,6-diazaspiro[3.3]heptan-6-yl]methanone;
[6-(5-cyclopropyl-4H-1,2,4-triazol-3-yl)-2-azaspiro[3.3]heptan-2-yl]-[2-(3,4-difluorophenyl)sulfonyl-2,6-diazaspiro[3.3]heptan-6-yl]methanone;
[2-(2-chloro-4-fluoro-phenyl)sulfonyl-2,6-diazaspiro[3.3]heptan-6-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[2-(2-methoxyphenyl)sulfonyl-2,6-diazaspiro[3.3]heptan-6-yl]methanone;
[6-(5-cyclopropyl-4H-1,2,4-triazol-3-yl)-2-azaspiro[3.3]heptan-2-yl]-[2-[[5-(trifluoromethyl)-3-pyridyl]sulfonyl]-2,6-diazaspiro[3.3]heptan-6-yl]methanone;
[6-(5-cyclopropyl-4H-1,2,4-triazol-3-yl)-2-azaspiro[3.3]heptan-2-yl]-[6-[[5-(trifluoromethyl)-2-pyridyl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[5-(trifluoromethyl)-2-pyridyl]methyl]-2-azaspiro[3.3]heptan-2-yl]methanone;
[6-(5-cyclobutyl-4H-1,2,4-triazol-3-yl)-2-azaspiro[3.3]heptan-2-yl]-[6-[[5-(trifluoromethyl)-2-pyridyl]methyl]-2-azaspiro[3.3]heptan-2-yl]methanone;
[6-[(5-chloro-3-pyridyl)methyl]-2-azaspiro[3.3]heptan-2-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[6-[[5-(trifluoromethyl)-3-pyridyl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[(3,5-difluoro-2-pyridyl)methyl]-2-azaspiro[3.3]heptan-2-yl]methanone;
[6-[(3,5-difluoro-2-pyridyl)methyl]-2-azaspiro[3.3]heptan-2-yl]-[6-[5-(1-hydroxycyclopropyl)-4H-1,2,4-triazol-3-yl]-2-azaspiro[3.3]heptan-2-yl]methanone;
[6-[(3-chloro-5-fluoro-2-pyridyl)methyl]-2-azaspiro[3.3]heptan-2-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[6-[[4-(trifluoromethyl)-2-pyridyl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[(5-methylsulfonyl-2-pyridyl)methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[(5-methylsulfonyl-3-pyridyl)methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[3-fluoro-5-(trifluoromethyl)phenyl]methyl]-2-azaspiro[3.3]heptan-2-yl]methanone;
[6-[[3-fluoro-5-(trifluoromethyl)phenyl]methyl]-2-azaspiro[3.3]heptan-2-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[6-[[4-(methylsulfonimidoyl)phenyl]methyl]-2-azaspiro[3.3]heptan-2-yl]methanone;
   [6-(5-cyclopropyl-4H-1,2,4-triazol-3-yl)-2-azaspiro[3.3]heptan-2-yl]-[6-[[3-(methylsulfonimidoyl)phenyl]methyl]-2-azaspiro[3.3]heptan-2-yl]methanone;
(rac)-[6-(5-cyclopropyl-4H-1,2,4-triazol-3-yl)-2-azaspiro[3.3]heptan-2-yl]-[6-[[3-(trifluoromethylsulfonimidoyl)phenyl]methyl]-2-azaspiro[3.3]heptan-2-yl]methanone;
(S)-[6-(5-cyclopropyl-4H-1,2,4-triazol-3-yl)-2-azaspiro[3.3]heptan-2-yl]-[6-[[3-(trifluoromethylsulfonimidoyl)phenyl]methyl]-2-azaspiro[3.3]heptan-2-yl]methanone;
(R)-[6-(5-cyclopropyl-4H-1,2,4-triazol-3-yl)-2-azaspiro[3.3]heptan-2-yl]-[6-[[3-(trifluoromethylsulfonimidoyl)phenyl]methyl]-2-azaspiro[3.3]heptan-2-yl]methanone;
(R)-[6-(5-cyclopropyl-4H-1,2,4-triazol-3-yl)-2-azaspiro[3.3]heptan-2-yl]-[6-[[4-(trifluoromethylsulfonimidoyl)phenyl]methyl]-2-azaspiro[3.3]heptan-2-yl]methanone;
(S)-[6-(5-cyclopropyl-4H-1,2,4-triazol-3-yl)-2-azaspiro[3.3]heptan-2-yl]-[6-[[4-(trifluoromethylsulfonimidoyl)phenyl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[(2-fluoro-4-methylsulfonyl-phenyl)methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[6-(trifluoromethyl)-3-pyridyl]oxy]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[6-(trifluoromethyl)-3-pyridyl]oxy]-2-azaspiro[3.3]heptan-2-yl]methanone;
[6-(5-cyclopropyl-4H-1,2,4-triazol-3-yl)-2-azaspiro[3.3]heptan-2-yl]-[6-[6-(trifluoromethyl)pyrazin-2-yl]oxy-2-azaspiro[3.3]heptan-2-yl]methanone;
[6-(5-cyclopropyl-4H-1,2,4-triazol-3-yl)-2-azaspiro[3.3]heptan-2-yl]-[7-[5-(trifluoromethyl)pyrimidin-2-yl]oxy-2-azaspiro[3.5]nonan-2-yl]methanone;
[6-(5-cyclopropyl-4H-1,2,4-triazol-3-yl)-2-azaspiro[3.3]heptan-2-yl]-[2-[[3-(trifluoromethyl)-1-bicyclo[1.1.1]pentanyl]sulfonyl]-2,6-diazaspiro[3.3]heptan-6-yl]methanone;
[6-(5-cyclopropyl-4H-1,2,4-triazol-3-yl)-2-azaspiro[3.3]heptan-2-yl]-[7-[[5-(trifluoromethyl)pyrazin-2-yl]methyl]-2-azaspiro[3.5]nonan-2-yl]methanone;
[6-[5-(1-hydroxycyclopropyl)-4H-1,2,4-triazol-3-yl]-2-azaspiro[3.3]heptan-2-yl]-[7-[[5-(trifluoromethyl)pyrazin-2-yl]methyl]-2-azaspiro[3.5]nonan-2-yl]methanone;
[7-[(5-chloro-2-pyridyl)methyl]-2-azaspiro[3.5]nonan-2-yl]-[6-(5-cyclopropyl-4H-1,2,4-triazol-3-yl)-2-azaspiro[3.3]heptan-2-yl]methanone;
[7-[(5-chloro-2-pyridyl)methyl]-2-azaspiro[3.5]nonan-2-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[7-[[6-(trifluoromethyl)-3-pyridyl]methyl]-2-azaspiro[3.5]nonan-2-yl]methanone;
[6-[5-(1-hydroxycyclopropyl)-4H-1,2,4-triazol-3-yl]-2-azaspiro[3.3]heptan-2-yl]-[7-[[6-(trifluoromethyl)-3-pyridyl]methyl]-2-azaspiro[3.5]nonan-2-yl]methanone;
[6-(5-cyclopropyl-4H-1,2,4-triazol-3-yl)-2-azaspiro[3.3]heptan-2-yl]-[7-[(3,5-difluoro-2-pyridyl)methyl]-2-azaspiro[3.5]nonan-2-yl]methanone;
[7-[(3,5-difluoro-2-pyridyl)methyl]-2-azaspiro[3.5]nonan-2-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[7-[(5-fluoro-2-pyridyl)methyl]-2-azaspiro[3.5]nonan-2-yl]methanone;
[7-[(5-fluoro-2-pyridyl)methyl]-2-azaspiro[3.5]nonan-2-yl]-[6-[5-(1-hydroxycyclopropyl)-4H-1,2,4-triazol-3-yl]-2-azaspiro[3.3]heptan-2-yl]methanone;
[7-[[6-(difluoromethoxy)-3-pyridyl]methyl]-2-azaspiro[3.5]nonan-2-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[6-[(3,5-difluoro-2-pyridyl)methyl]-2-azaspiro[3.4]octan-2-yl]methanone:
[6-(5-cyclopropyl-4H-1,2,4-triazol-3-yl)-2-azaspiro[3.3]heptan-2-yl]-[rac-(6S)-6-[(3,5-difluoro-2-pyridyl)methyl]-2-azaspiro[3.4]octan-2-yl]methanone;
[6-(5-cyclopropyl-4H-1,2,4-triazol-3-yl)-2-azaspiro[3.3]heptan-2-yl]-[rac-(6R)-6-[(3,5-difluoro-2-pyridyl)methyl]-2-azaspiro[3.4]octan-2-yl]methanone;
[6-(5-cyclopropyl-4H-1,2,4-triazol-3-yl)-2-azaspiro[3.3]heptan-2-yl]-[rac-(6R)-6-[[5-(trifluoromethyl)pyrazin-2-yl]methyl]-2-azaspiro[3.4]octan-2-yl]methanone;
[6-(5-cyclopropyl-4H-1,2,4-triazol-3-yl)-2-azaspiro[3.3]heptan-2-yl]-[rac-(6S)-6-[[5-(trifluoromethyl)pyrazin-2-yl]methyl]-2-azaspiro[3.4]octan-2-yl]methanone;
[6-(5-cyclopropyl-4H-1,2,4-triazol-3-yl)-2-azaspiro[3.3]heptan-2-yl]-[6-[[6-(trifluoromethyl)-3-pyridyl]methyl]-2-azaspiro[3.4]octan-2-yl]methanone;
[6-(5-cyclopropyl-4H-1,2,4-triazol-3-yl)-2-azaspiro[3.3]heptan-2-yl]-[6-[[4-methylsulfonyl-3-(trifluoromethyl)phenyl]methyl]-2-azaspiro[3.4]octan-2-yl]methanone;
5-[[rac-(6S)-2-[6-(5-cyclopropyl-4H-1,2,4-triazol-3-yl)-2-azaspiro[3.3]heptane-2-carbonyl]-2-azaspiro[3.4]octan-6-yl]oxy]-2-(trifluoromethyl)pyridine-4-carbonitrile;
[6-(5-cyclopropyl-4H-1,2,4-triazol-3-yl)-2-azaspiro[3.3]heptan-2-yl]-[rac-(6S)-6-[4-methylsulfonyl-3-(trifluoromethyl)phenoxy]-2-azaspiro[3.4]octan-2-yl]methanone;
[6-(5-cyclopropyl-4H-1,2,4-triazol-3-yl)-2-azaspiro[3.3]heptan-2-yl]-[6-[(2,4-difluorophenyl)methyl]-2,6-diazaspiro[3.3]heptan-2-yl]methanone;
[6-[(2,4-difluorophenyl)methyl]-2,6-diazaspiro[3.3]heptan-2-yl]-[6-[5-(1-hydroxycyclopropyl)-4H-1,2,4-triazol-3-yl]-2-azaspiro[3.3]heptan-2-yl]methanone;
[6-(5-cyclobutyl-4H-1,2,4-triazol-3-yl)-2-azaspiro[3.3]heptan-2-yl]-[6-[(2,4-difluorophenyl)methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[(3,5-difluorophenyl)methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[[6-(trifluoromethyl)pyridazin-3-yl]methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[[5-(trifluoromethyl)pyrimidin-2-yl]methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[[2-(trifluoromethyl)pyrimidin-5-yl]methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[[5-(trifluoromethyl)-2-pyridyl]methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[[5-(trifluoromethyl)pyrazin-2-yl]methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[(3,5-difluoro-2-pyridyl)methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[(4-methylsulfonylphenyl)methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[(3-methylsulfonylphenyl)methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[(2-methylsulfonylphenyl)methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[[4-fluoro-2-(trifluoromethyl)phenyl]methyl]-2,6-diazaspiro[3.3]heptan-2-yl]methanone;
2-[[2-[6-(5-cyclopropyl-4H-1,2,4-triazol-3-yl)-2-azaspiro[3.3]heptane-2-carbonyl]-2,6-diazaspiro[3.3]heptan-6-yl]methyl]benzenesulfonamide;
methyl 2-[[2-[6-(5-cyclopropyl-4H-1,2,4-triazol-3-yl)-2-azaspiro[3.3]heptane-2-carbonyl]-2,6-diazaspiro[3.3]heptan-6-yl]methyl]benzoate;
2-[[2-[6-(5-cyclopropyl-4H-1,2,4-triazol-3-yl)-2-azaspiro[3.3]heptane-2-carbonyl]-2,6-diazaspiro[3.3]heptan-6-yl]methyl]benzoic acid;
2-[[2-[6-(5-cyclopropyl-4H-1,2,4-triazol-3-yl)-2-azaspiro[3.3]heptane-2-carbonyl]-2,6-diazaspiro[3.3]heptan-6-yl]methyl]-N-methyl-benzamide;
[6-(5-cyclopropyl-4H-1,2,4-triazol-3-yl)-2-azaspiro[3.3]heptan-2-yl]-[6-[[2-[1-(methoxymethyl)-1,2,4-triazol-3-yl]phenyl]methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[[2-[1-(methoxymethyl)imidazol-2-yl]phenyl]methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[7-[[6-(trifluoromethyl)-3-pyridyl]methyl]-2,7-diazaspiro[3.5]nonan-2-yl]methanone;
[6-(5-cyclopropyl-4H-1,2,4-triazol-3-yl)-2-azaspiro[3.3]heptan-2-yl]-[7-[[2-fluoro-4-(trifluoromethyl)phenyl]methyl]-2,7-diazaspiro[3.5]nonan-2-yl]methanone;
[6-(5-cyclopropyl-4H-1,2,4-triazol-3-yl)-2-azaspiro[3.3]heptan-2-yl]-[7-[[5-(trifluoromethyl)-2-pyridyl]methyl]-2,7-diazaspiro[3.5]nonan-2-yl]methanone;
[6-(5-cyclopropyl-4H-1,2,4-triazol-3-yl)-2-azaspiro[3.3]heptan-2-yl]-[7-[(4-methylsulfonylphenyl)methyl]-2,7-diazaspiro[3.5]nonan-2-yl]methanone;
[6-(5-cyclopropyl-4H-1,2,4-triazol-3-yl)-2-azaspiro[3.3]heptan-2-yl]-[7-[[3-(trifluoromethylsulfonyl)phenyl]methyl]-2,7-diazaspiro[3.5]nonan-2-yl]methanone;
[6-(5-cyclopropyl-4H-1,2,4-triazol-3-yl)-2-azaspiro[3.3]heptan-2-yl]-[7-[(3-methylsulfonylphenyl)methyl]-2,7-diazaspiro[3.5]nonan-2-yl]methanone;
[6-(5-cyclopropyl-4H-1,2,4-triazol-3-yl)-2-azaspiro[3.3]heptan-2-yl]-[7-[[5-(trifluoromethyl)-2-pyridyl]methyl]-2,7-diazaspiro[3.4]octan-2-yl]methanone;
[6-(5-cyclopropyl-4H-1,2,4-triazol-3-yl)-2-azaspiro[3.3]heptan-2-yl]-[7-[[6-(trifluoromethyl)pyridazin-3-yl]methyl]-2,7-diazaspiro[3.4]octan-2-yl]methanone;
[6-(5-cyclopropyl-4H-1,2,4-triazol-3-yl)-2-azaspiro[3.3]heptan-2-yl]-[7-[[5-(trifluoromethyl)pyrazin-2-yl]methyl]-2,7-diazaspiro[3.4]octan-2-yl]methanone;
[6-(5-cyclopropyl-4H-1,2,4-triazol-3-yl)-2-azaspiro[3.3]heptan-2-yl]-[6-[[2-fluoro-4-(trifluoromethyl)phenyl]methy (amino]-2-azaspiro[3.3]heptan-2-yl]methanone;
[6-(5-cyclopropyl-4H-1,2,4-triazol-3-yl)-2-azaspiro[3.3]heptan-2-yl]-[3-[[4-(trifluoromethyl)phenyl]methylamino]azetidin-1-yl]methanone;
[6-(5-cyclopropyl-4H-1,2,4-triazol-3-yl)-2-azaspiro[3.3]heptan-2-yl]-[3-[[5-fluoro-2-(trifluoromethyl)phenyl]methylamino]azetidin-1-yl]methanone;
[3-[[2-chloro-5-(trifluoromethyl)phenyl]methylamino]azetidin-1-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[3-[[2-fluoro-4-(trifluoromethoxy)phenyl]methylamino]azetidin-1-yl]methanone;
[3-[[2-chloro-4-(trifluoromethyl)phenyl]methylamino]azetidin-1-yl]-[6-(5-cyclopropyl-4H-1,2,4-triazol-3-yl)-2-azaspiro[3.3]heptan-2-yl]methanone:
[6-(5-cyclopropyl-4H-1,2,4-triazol-3-yl)-2-azaspiro[3.3]heptan-2-yl]-[3-[[4-(trifluoromethoxy)phenyl]methylamino]azetidin-1-yl]methanone;
[6-(5-cyclopropyl-4H-1,2,4-triazol-3-yl)-2-azaspiro[3.3]heptan-2-yl]-[3-[[2-fluoro-4-(trifluoromethyl)phenyl]methylamino]azetidin-1-yl]methanone;
[6-(5-cyclopropyl-4H-1,2,4-triazol-3-yl)-2-azaspiro[3.3]heptan-2-yl]-[3-[[3-fluoro-4-(trifluoromethyl)phenyl]methylamino]azetidin-1-yl]methanone;
[3-[[3-chloro-5-(trifluoromethyl)phenyl]methylamino]azetidin-1-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[3-[[2-fluoro-5-(trifluoromethyl)phenyl]methylamino]azetidin-1-yl]methanone:
[6-(5-cyclopropyl-4H-1,2,4-triazol-3-yl)-2-azaspiro[3.3]heptan-2-yl]-[3-[[4-fluoro-2-(trifluoromethyl)phenyl]methylamino]azetidin-1-yl]methanone;
[6-(5-cyclopropyl-4H-1,2,4-triazol-3-yl)-2-azaspiro[3.3]heptan-2-yl]-[3-[[2-methoxy-4-(trifluoromethyl)phenyl]methylamino]azetidin-1-yl]methanone;
[3-[[2-fluoro-4-(trifluoromethyl)phenyl]methylamino]azetidin-1-yl]-[6-[5-(1-hydroxycyclopropyl)-4H-1,2,4-triazol-3-yl]-2-azaspiro[3.3]heptan-2-yl]methanone;
[3-[[5-chloro-2-(trifluoromethyl)phenyl]methylamino]azetidin-1-yl]-[6-(5-cyclopropyl-4H-1,2,4-triazol-3-yl)-2-azaspiro[3.3]heptan-2-yl]methanone;
[3-[cyclopropyl-[[2-fluoro-4-(trifluoromethyl)phenyl]methyl]amino]azetidin-1-yl]-[6-(5-cyclopropyl-4H-1,2,4-triazol-3-yl)-2-azaspiro[3.3]heptan-2-yl]methanone;
[3-[cyclopropylmethyl-[[2-fluoro-4-(trifluoromethyl)phenyl]methyl]amino]azetidin-1-yl]-[6-(5-cyclopropyl-4H-1,2,4-triazol-3-yl)-2-azaspiro[3.3]heptan-2-yl]methanone;
N-[1-[6-(5-cyclopropyl-4H-1,2,4-triazol-3-yl)-2-azaspiro[3.3]heptane-2-carbonyl]azetidin-3-yl]-3-(trifluoromethyl)benzenesulfonamide;
N-[1-[6-(5-cyclopropyl-4H-1,2,4-triazol-3-yl)-2-azaspiro[3.3]heptane-2-carbonyl]azetidin-3-yl]-2-fluoro-4-(trifluoromethyl)benzenesulfonamide;
N-[1-[6-(5-cyclopropyl-4H-1,2,4-triazol-3-yl)-2-azaspiro[3.3]heptane-2-carbonyl]azetidin-3-yl]-4,4-difluoro-cyclohexanesulfonamide;
N-[1-[6-(5-cyclopropyl-4H-1,2,4-triazol-3-yl)-2-azaspiro[3.3]heptane-2-carbonyl]azetidin-3-yl]-3-(trifluoromethoxy)benzenesulfonamide;
[6-(5-cyclobutyl-4H-1,2,4-triazol-3-yl)-2-azaspiro[3.3]heptan-2-yl]-[3-[[2-fluoro-4-(trifluoromethyl)phenyl]methylamino]azetidin-1-yl]methanone;
[6-(5-cyclopropyl-4H-1,2,4-triazol-3-yl)-2-azaspiro[3.3]heptan-2-yl]-[3-[4-(2,2,2-trifluoroethoxy)pyrazol-1-yl]azetidin-1-yl]methanone;
N-[[1-[6-(5-cyclopropyl-4H-1,2,4-triazol-3-yl)-2-azaspiro[3.3]heptane-2-carbonyl]azetidin-3-yl]methyl]-3-(trifluoromethyl)benzenesulfonamide;
N-[[1-[6-(5-cyclopropyl-4H-1,2,4-triazol-3-yl)-2-azaspiro[3.3]heptane-2-carbonyl]azetidin-3-yl]methyl]-4-(trifluoromethyl)benzenesulfonamide;
N-[[1-[6-[5-(1-hydroxycyclopropyl)-4H-1,2,4-triazol-3-yl]-2-azaspiro[3.3]heptane-2-carbonyl]azetidin-3-yl]methyl]-4-(trifluoromethyl)benzenesulfonamide;
N-[[1-[6-[5-(1-hydroxycyclopropyl)-4H-1,2,4-triazol-3-yl]-2-azaspiro[3.3]heptane-2-carbonyl]azetidin-3-yl]methyl]-3-(trifluoromethyl)benzenesulfonamide;
[6-(5-cyclopropyl-4H-1,2,4-triazol-3-yl)-2-azaspiro[3.3]heptan-2-yl]-[2-(2,2-dimethylpropylsulfonyl)-2,6-diazaspiro[3.3]heptan-6-yl]methanone;
6-[6-(5-cyclopropyl-4H-1,2,4-triazol-3-yl)-2-azaspiro[3.3]heptane-2-carbonyl]-N-(1-methylcyclopropyl)-2,6-di azaspiro[3.3]heptane-2-sulfonamide;
6-[6-(5-cyclopropyl-4H-1,2,4-triazol-3-yl)-2-azaspiro[3.3]heptane-2-carbonyl]-N-[(4-fluorophenyl)methyl]-2,6-diazaspiro[3.3]heptane-2-sulfonamide;
6-[6-(5-cyclopropyl-4H-1,2,4-triazol-3-yl)-2-azaspiro[3.3]heptane-2-carbonyl]-N-[1-(trifluoromethyl)cyclopropyl]-2,6-diazaspiro[3.3]heptane-2-sulfonamide;
6-[6-(5-cyclopropyl-4H-1,2,4-triazol-3-yl)-2-azaspiro[3.3]heptane-2-carbonyl]-N-[[1-(trifluoromethyl)cyclopropyl]methyl]-2,6-diazaspiro[3.3]heptane-2-sulfonamide;
1-[2-[6-(5-cyclopropyl-4H-1,2,4-triazol-3-yl)-2-azaspiro[3.3]heptane-2-carbonyl]-2,6-diazaspiro[3.3]heptan-6-yl]-2,2,2-trifluoro-ethanone;
[6-(5-cyclopropyl-4H-1,2,4-triazol-3-yl)-2-azaspiro[3.3]heptan-2-yl]-[6-[5-(trifluoromethyl)pyrazin-2-yl]-2,6-diazaspiro[3.3]heptan-2-yl]methanone;
[6-(5-cyclopropyl-4H-1,2,4-triazol-3-yl)-2-azaspiro[3.3]heptan-2-yl]-[3-[(4,4-difluoro-1-piperidyl)methyl]azetidin-1-yl]methanone;
[6-(5-cyclopropyl-4H-1,2,4-triazol-3-yl)-2-azaspiro[3.3]heptan-2-yl]-[6-(1,1-dioxo-1,4-thiazinan-4-yl)-2-azaspiro[3.3]heptan-2-yl]methanone;
[6-(5-cyclopropyl-4H-1,2,4-triazol-3-yl)-2-azaspiro[3.3]heptan-2-yl]-[6-[[1-(trifluoromethyl)cyclopropyl]methylamino]-2-azaspiro[3.3]heptan-2-yl]methanone;
N-[2-[6-(5-cyclopropyl-4H-1,2,4-triazol-3-yl)-2-azaspiro[3.3]heptane-2-carbonyl]-2-azaspiro[3.3]heptan-6-yl]-1-(trifluoromethyl)cyclopropanecarboxamide;
N-[2-[6-(5-cyclopropyl-4H-1,2,4-triazol-3-yl)-2-azaspiro[3.3]heptane-2-carbonyl]-2-azaspiro[3.3]heptan-6-yl]-4-(trifluoromethyl)benzenesulfonamide;
N-[2-[6-(5-cyclopropyl-4H-1,2,4-triazol-3-yl)-2-azaspiro[3.3]heptane-2-carbonyl]-2-azaspiro[3.3]heptan-6-yl]-2-(trifluoromethyl)benzenesulfonamide;
N-[2-[6-(5-cyclopropyl-4H-1,2,4-triazol-3-yl)-2-azaspiro[3.3]heptane-2-carbonyl]-2-azaspiro[3.3]heptan-6-yl]-2,2-dimethyl-propane-1-sulfonamide;
[6-(5-cyclopropyl-4H-1,2,4-triazol-3-yl)-2-azaspiro[3.3]heptan-2-yl]-[3-[4-[1-(trifluoromethyl)cyclopropyl]phenyl]azetidin-1-yl]methanone;
[6-[5-(1-hydroxycyclopropyl)-4H-1,2,4-triazol-3-yl]-2-azaspiro[3.3]heptan-2-yl]-[3-[4-[1-(trifluoromethyl)cyclopropyl]phenyl]azetidin-1-yl]methanone;
[6-[5-(2,2,2-trifluoro-1-hydroxy-ethyl)-4H-1,2,4-triazol-3-yl]-2-azaspiro[3.3]heptan-2-yl]-[3-[4-[1-(trifluoromethyl)cyclopropyl]phenyl]azetidin-1-yl]methanone;
[6-[5-[(1R)-2,2,2-trifluoro-1-hydroxy-ethyl]-4H-1,2,4-triazol-3-yl]-2-azaspiro[3.3]heptan-2-yl]-[3-[4-[1-(trifluoromethyl)cyclopropyl]phenyl]azetidin-1-yl]methanone:
[6-[5-[(1 S)-2,2,2-trifluoro-1-hydroxy-ethyl]-4H-1,2,4-triazol-3-yl]-2-azaspiro[3.3]heptan-2-yl]-[3-[4-[1-(trifluoromethyl)cyclopropyl]phenyl]azetidin-1-yl]methanone;
[6-(5-cyclopropyl-4H-1,2,4-triazol-3-yl)-2-azaspiro[3.3]heptan-2-yl]-[3-[6-[1-(trifluoromethyl)cyclopropyl]-3-pyridyl]azetidin-1-yl]methanone;
[6-[5-(1-hydroxycyclopropyl)-4H-1,2,4-triazol-3-yl]-2-azaspiro[3.3]heptan-2-yl]-[3-[6-[1-(trifluoromethyl)cyclopropyl]-3-pyridyl]azetidin-1-yl]methanone;
[6-(5-cyclopropyl-4H-1,2,4-triazol-3-yl)-2-azaspiro[3.3]heptan-2-yl]-[3-[5-[1-(trifluoromethyl)cyclopropyl]pyrazin-2-yl]azetidin-1-yl]methanone;
[6-(5-cyclopropyl-4H-1,2,4-triazol-3-yl)-2-azaspiro[3.3]heptan-2-yl]-[3-[6-[1-(trifluoromethyl)cyclopropyl]pyridazin-3-yl]azetidin-1-yl]methanone;
[6-(5-cyclopropyl-4H-1,2,4-triazol-3-yl)-2-azaspiro[3.3]heptan-2-yl]-[3-[2-[1-(trifluoromethyl)cyclopropyl]pyrimidin-5-yl]azetidin-1-yl]methanone;
[6-(5-cyclopropyl-4H-1,2,4-triazol-3-yl)-2-azaspiro[3.3]heptan-2-yl]-[3-[5-[1-(trifluoromethyl)cyclopropyl]-2-pyridyl]azetidin-1-yl]methanone;
[6-(5-cyclopropyl-4H-1,2,4-triazol-3-yl)-2-azaspiro[3.3]heptan-2-yl]-[7-[[3-(trifluoromethyl)-1-bicyclo[1.1.1]pentanyl]sulfonyl]-2,7-diazaspiro[3.5]nonan-2-yl]methanone;
[6-[5-(1-hydroxycyclopropyl)-4H-1,2,4-triazol-3-yl]-2-azaspiro[3.3]heptan-2-yl]-[7-[[3-(trifluoromethyl)-1-bicyclo[1.1.1]pentanyl]sulfonyl]-2,7-diazaspiro[3.5]nonan-2-yl]methanone;
[6-(5-cyclobutyl-4H-1,2,4-triazol-3-yl)-2-azaspiro[3.3]heptan-2-yl]-[7-[[3-(trifluoromethyl)-1-bicyclo[1.1.1]pentanyl]sulfonyl]-2,7-diazaspiro[3.5]nonan-2-yl]methanone;
[6-(5-cyclopropyl-4H-1,2,4-triazol-3-yl)-2-azaspiro[3.3]heptan-2-yl]-[7-[2-fluoro-4-(trifluoromethyl)phenyl]sulfonyl-2,7-diazaspiro[3.5]nonan-2-yl]methanone;
[6-(5-cyclopropyl-4H-1,2,4-triazol-3-yl)-2-azaspiro[3.3]heptan-2-yl]-[3-[3-fluoro-4-(trifluoromethoxy)phenyl]azetidin-1-yl]methanone;
[3-[3-fluoro-4-(trifluoromethoxy)phenyl]azetidin-1-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[3-[4-(trifluoromethylsulfonyl)phenyl]azetidin-1-yl]methanone;
[6-[5-(1-hydroxycyclopropyl)-4H-1,2,4-triazol-3-yl]-2-azaspiro[3.3]heptan-2-yl]-[3-[4-(2,2,2-trifluoroethyl)phenyl]azetidin-1-yl]methanone;
[6-(5-cyclopropyl-4H-1,2,4-triazol-3-yl)-2-azaspiro[3.3]heptan-2-yl]-[3-[3-[[1-(trifluoromethyl)cyclopropyl]methyl]-1,2,4-oxadiazol-5-yl]azetidin-1-yl]methanone;
[6-(5-cyclopropyl-4H-1,2,4-triazol-3-yl)-2-azaspiro[3.3]heptan-2-yl]-[3-[5-[1-(trifluoromethyl)cyclopropyl]-1,2,4-oxadiazol-3-yl]azetidin-1-yl]methanone;
[3-(2-chloro-4-isopropylsulfonyl-phenyl)azetidin-1-yl]-[6-(5-cyclopropyl-4H-1,2,4-triazol-3-yl)-2-azaspiro[3.3]heptan-2-yl]methanone;
N-[1-[6-(5-cyclopropyl-4H-1,2,4-triazol-3-yl)-2-azaspiro[3.3]heptane-2-carbonyl]-4-piperidyl]-4-(trifluoromethyl)benzenesulfonamide;
N-[1-[6-(5-cyclopropyl-4H-1,2,4-triazol-3-yl)-2-azaspiro[3.3]heptane-2-carbonyl]-4-piperidyl]-4-fluoro-benzenesulfonamide;
N-[[1-[6-(5-cyclopropyl-4H-1,2,4-triazol-3-yl)-2-azaspiro[3.3]heptane-2-carbonyl]-4-piperidyl]methyl]-4-(trifluoromethyl)benzenesulfonamide;
N-[[1-[6-(S-cyclopropyl-4H-1,2,4-triazol-3-yl)-2-azaspiro[3.3]heptane-2-carbonyl]-4-piperidyl]methyl]-4-(trifluoromethoxy)benzenesulfonamide;
4-chloro-N-[[1-[6-(5-cyclopropyl-H-1,2,4-triazol-3-yl)-2-azaspiro[3.3]heptane-2-carbonyl]-4-piperidyl]methyl]benzenesulfonamide;
[6-(5-cyclopropyl-4H-1,2,4-triazol-3-yl)-2-azaspiro[3.3]heptan-2-yl]-[3-[6-[[1-(trifluoromethyl)cyclopropyl]methylamino]-3-pyridyl]azetidin-1-yl]methanone;
[6-[5-(1-hydroxycyclopropyl)-4H-1,2,4-triazol-3-yl]-2-azaspiro[3.3]heptan-2-yl]-[3-[6-[[1-(trifluoromethyl)cyclopropyl]methylamino]-3-pyridyl]azetidin-1-yl]methanone;
[6-(5-cyclopropyl-4H-1,2,4-triazol-3-yl)-2-azaspiro[3.3]heptan-2-yl]-[3-[5-[[1-(trifluoromethyl)cyclopropyl]methylamino]pyrazin-2-yl]azetidin-1-yl]methanone;
[6-(5-cyclopropyl-4H-1,2,4-triazol-3-yl)-2-azaspiro[3.3]heptan-2-yl]-[3-[5-[[1-(trifluoromethyl)cyclopropyl]methylamino]-2-pyridyl]azetidin-1-yl]methanone;
[6-(5-cyclopropyl-4H-1,2,4-triazol-3-yl)-2-azaspiro[3.3]heptan-2-yl]-[3-[4-[[1-(trifluoromethyl)cyclopropyl]methylamino]phenyl]azetidin-1-yl]methanone;
[6-(5-cyclopropyl-4H-1,2,4-triazol-3-yl)-2-azaspiro[3.3]heptan-2-yl]-[3-[6-[3-(trifluoromethyl)azetidin-1-yl]-3-pyridyl]azetidin-1-yl]methanone;
[6-[5-(1-hydroxycyclopropyl)-4H-1,2,4-triazol-3-yl]-2-azaspiro[3.3]heptan-2-yl]-[3-[6-[3-(trifluoromethyl)azetidin-1-yl]-3-pyridyl]azetidin-1-yl]methanone;
[6-(5-cyclobutyl-4H-1,2,4-triazol-3-yl)-2-azaspiro[3.3]heptan-2-yl]-[3-[6-[3-(trifluoromethyl)azetidin-1-yl]-3-pyridyl]azetidin-1-yl]methanone;
[6-(5-cyclopropyl-4H-1,2,4-triazol-3-yl)-2-azaspiro[3.3]heptan-2-yl]-[3-[4-[3-(trifluoromethyl)azetidin-1-yl]phenyl]azetidin-1-yl]methanone:
[6-[5-(1-hydroxycyclopropyl)-4H-1,2,4-triazol-3-yl]-2-azaspiro[3.3]heptan-2-yl]-[3-[4-[3-(trifluoromethyl)azetidin-1-yl]phenyl]azetidin-1-yl]methanone;
[6-(5-cyclopropyl-4H-1,2,4-triazol-3-yl)-2-azaspiro[3.3]heptan-2-yl]-[3-[2-[3-(trifluoromethyl)azetidin-1-yl]pyrimidin-5-yl]azetidin-1-yl]methanone;
[6-(5-cyclopropyl-4H-1,2,4-triazol-3-yl)-2-azaspiro[3.3]heptan-2-yl]-[3-[2-[3-(trifluoromethyl)pyrrolidin-1-yl]pyrimidin-5-yl]azetidin-1-yl]methanone;
[6-(5-cyclopropyl-4H-1,2,4-triazol-3-yl)-2-azaspiro[3.3]heptan-2-yl]-[3-[2-[(3R)-3-(trifluoromethyl)pyrrolidin-1-yl]pyrimidin-5-yl]azetidin-1-yl]methanone;
[6-(5-cyclopropyl-4H-1,2,4-triazol-3-yl)-2-azaspiro[3.3]heptan-2-yl]-[3-[2-[(3S)-3-(trifluoromethyl)pyrrolidin-1-yl]pyrimidin-5-yl]azetidin-1-yl]methanone;
[6-(5-cyclopropyl-4H-1,2,4-triazol-3-yl)-2-azaspiro[3.3]heptan-2-yl]-[3-[6-[(3S)-3-(trifluoromethyl)pyrolidin-1-yl]-3-pyridyl]azetidin-1-yl]methanone;
[6-(5-cyclopropyl-4H-1,2,4-triazol-3-yl)-2-azaspiro[3.3]heptan-2-yl]-[3-[6-[(3R)-3-(trifluoromethyl)pyrrolidin-1-yl]-3-pyridyl]azetidin-1-yl]methanone;
[6-(5-cyclopropyl-4H-1,2,4-triazol-3-yl)-2-azaspiro[3.3]heptan-2-yl]-[3-[6-[3-hydroxy-3-(trifluoromethyl)pyrrolidin-1-yl]-3-pyridyl]azetidin-1-yl]methanone;
[6-(5-cyclopropyl-4H-1,2,4-triazol-3-yl)-2-azaspiro[3.3]heptan-2-yl]-[3-[6-[rac-(3R)-3-hydroxy-3-(trifluoromethyl)pyrrolidin-1-yl]-3-pyridyl]azetidin-1-yl]methanone;
[6-(5-cyclopropyl-4H-1,2,4-triazol-3-yl)-2-azaspiro[3.3]heptan-2-yl]-[3-[6-[rac-(3S)-3-hydroxy-3-(trifluoromethyl)pyrrolidin-1-yl]-3-pyridyl]azetidin-1-yl]methanone;
[6-[5-(1-hydroxycyclopropyl)-4H-1,2,4-triazol-3-yl]-2-azaspiro[3.3]heptan-2-yl]-[3-[6-[[1-(trifluoromethyl)cyclopropyl]amino]-3-pyridyl]azetidin-1-yl]methanone;
[6-(5-cyclobutyl-4H-1,2,4-triazol-3-yl)-2-azaspiro[3.3]heptan-2-yl]-[3-[6-[[1-(trifluoromethyl)cyclopropyl]amino]-3-pyridyl]azetidin-1-yl]methanone;
[6-(5-cyclopropyl-4H-1,2,4-triazol-3-yl)-2-azaspiro[3.3]heptan-2-yl]-[3-[4-[[1-(trifluoromethyl)cyclopropyl]amino]phenyl]azetidin-1-yl]methanone;
[6-(5-cyclopropyl-4H-1,2,4-triazol-3-yl)-2-azaspiro[3.3]heptan-2-yl]-[3-[6-[3-hydroxy-3-(trifluoromethyl)azetidin-1-yl]-3-pyridyl]azetidin-1-yl]methanone;
[6-(5-Cyclopropyl-4H-1,2,4-triazol-3-yl)-2-azaspiro[3.3]heptan-2-yl]-[3-[4-(3,5-diisopropylpyrazol-1-yl)phenyl]azetidin-1-yl]methanone;
[3-[4-(3-tert-Butyl-5-methyl-pyrazol-1-yl)phenyl]azetidin-1-yl]-[6-(5-cyclopropyl-4H-1,2,4-triazol-3-yl)-2-azaspiro[3.3]heptan-2-yl]methanone;
[6-(5-Cyclopropyl-4H-1,2,4-triazol-3-yl)-2-azaspiro[3.3]heptan-2-yl]-[3-[4-[5-methyl-3-(trifluoromethyl)pyrazol-1-yl]phenyl]azetidin-1-yl]methanone;
(4R) or (4S)-1-[4-[1-[6-(5-Cyclopropyl-4H-1,2,4-triazol-3-yl)-2-azaspiro[3.3]heptane-2-carbonyl]azetidin-3-yl]phenyl]-4-(trifluoromethyl)piperidin-2-one;
(4S) or (4R)-1-[4-[1-[6-(5-Cyclopropyl-4H-1,2,4-triazol-3-yl)-2-azaspiro[3.3]heptane-2-carbonyl]azetidin-3-yl]phenyl]-4-(trifluoromethyl)piperidin-2-one;
[7-(5-Chloro-3-fluoro-2-pyridyl)-2,7-diazaspiro[3.5]nonan-2-yl]-[6-(5-cyclopropyl-4H-1,2,4-triazol-3-yl)-2-azaspiro[3.3]heptan-2-yl]methanone;
[3-[4-[3-(Difluoromethyl)cyclobutyl]phenyl]azetidin-1-yl]-[6-[5-(1-hydroxycyclopropyl)-4H-1,2,4-triazol-3-yl]-2-azaspiro[3.3]heptan-2-yl]methanone;
[7-(4-Chloro-2-fluoro-phenyl)-2,7-diazaspiro[3.5]nonan-2-yl]-[6-(5-cyclopropyl-4H-1,2,4-triazol-3-yl)-2-azaspiro[3.3]heptan-2-yl]methanone;
1-[4-[1-[6-(5-Cyclopropyl-4H-1,2,4-triazol-3-yl)-2-azaspiro[3.3]heptane-2-carbonyl]azetidin-3-yl]phenyl]-4-(trifluoromethyl)pyridin-2-one;
[6-(5-Cyclopropyl-4H-1,2,4-triazol-3-yl)-2-azaspiro[3.3]heptan-2-yl]-[3-[4-[3-(difluoromethyl)-5-methyl-pyrazol-1-yl]phenyl]azetidin-1-yl]methanone;
[6-(5-Cyclopropyl-4H-1,2,4-triazol-3-yl)-2-azaspiro[3.3]heptan-2-yl]-[3-(5-spiro[3.3]heptan-2-ylpyrazin-2-yl)azetidin-1-yl]methanone;
[3-[4-(5-Cyclopropyl-3-methyl-pyrazol-1-yl)phenyl]azetidin-1-yl]-[6-(5-cyclopropyl-4H-1,2,4-triazol-3-yl)-2-azaspiro[3.3]heptan-2-yl]methanone;
[3-[4-(3-Cyclopropyl-5-methyl-pyrazol-1-yl)phenyl]azetidin-1-yl]-[6-(5-cyclopropyl-4H-1,2,4-triazol-3-yl)-2-azaspiro[3.3]heptan-2-yl]methanone;
[6-(5-Cyclopropyl-4H-1,2,4-triazol-3-yl)-2-azaspiro[3.3]heptan-2-yl]-[3-[6-(2,2-difluoro-5-azaspiro[2.4]heptan-5-yl)-3-pyridyl]azetidin-1-yl]methanone;
[6-(5-Cyclopropyl-4H-1,2,4-triazol-3-yl)-2-azaspiro[3.3]heptan-2-yl]-[3-[6-(6-oxa-2-azaspiro[3.4]octan-2-yl)-3-pyridyl]azetidin-1-yl]methanone;
[6-(5-Cyclopropyl-4H-1,2,4-triazol-3-yl)-2-azaspiro[3.3]heptan-2-yl]-[3-[4-[3-hydroxy-3-(trifluoromethyl)cyclobutyl]phenyl]azetidin-1-yl]methanone;
[6-(5-Cyclopropyl-4H-1,2,4-triazol-3-yl)-2-azaspiro[3.3]heptan-2-yl]-[3-[5-[3-(trifluoromethyl)azetidin-1-yl]-2-pyridyl]azetidin-1-yl]methanone;
[6-(5-Cyclopropyl-4H-1,2,4-triazol-3-yl)-2-azaspiro[3.3]heptan-2-yl]-[3-[6-(5-oxa-2-azaspiro[3.4]octan-2-yl)-3-pyridyl]azetidin-1-yl]methanone;
[6-(5-Cyclopropyl-4H-1,2,4-triazol-3-yl)-2-azaspiro[3.3]heptan-2-yl]-[3-[4-(3,5-dimethylpyrazol-1-yl)phenyl]azetidin-1-yl]methanone;
[6-(5-Cyclopropyl-4H-1,2,4-triazol-3-yl)-2-azaspiro[3.3]heptan-2-yl]-[3-[4-[1-(hydroxymethyl)cyclopropyl]phenyl]azetidin-1-yl]methanone:
[6-(5-Cyclopropyl-4H-1,2,4-triazol-3-yl)-2-azaspiro[3.3]heptan-2-yl]-[3-[6-(2-oxaspiro[3.3]heptan-6-yl)-3-pyridyl]azetidin-1-yl]methanone; 4-methylbenzenesulfonic acid;
[3-(4-Cyclobutyl-3-methylsulfonyl-phenyl)azetidin-1-yl]-[6-(5-cyclopropyl-4H-1,2,4-triazol-3-yl)-2-azaspiro[3.3]heptan-2-yl]methanone;
[6-(5-Cyclopropyl-4H-1,2,4-triazol-3-yl)-2-azaspiro[3.3]heptan-2-yl]-[3-[6-[3-(1-hydroxy-1-methyl-ethyl)azetidin-1-yl]-3-pyridyl]azetidin-1-yl]methanone;
[6-(5-Cyclopropyl-4H-1,2,4-triazol-3-yl)-2-azaspiro[3.3]heptan-2-yl]-[3-[6-(2,2-dimethylpropyl)-3-pyridyl]azetidin-1-yl]methanone;
(trans)-[6-(5-Cyclopropyl-4H-1,2,4-triazol-3-yl)-2-azaspiro[3.3]heptan-2-yl]-[3-[6-[3-(trifluoromethyl)cyclobutyl]-3-pyridyl]azetidin-1-yl]methanone;
(trans)-[6-(5-Cyclopropyl-4H-1,2,4-triazol-3-yl)-2-azaspiro[3.3]heptan-2-yl]-[3-[6-[3-(difluoromethyl)cyclobutyl]-3-pyridyl]azetidin-1-yl]methanone;
[3-[6-(2-Azaspiro[3.4]octan-2-yl)-3-pyridyl]azetidin-1-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[3-[6-(2-oxa-7-azaspiro[3.4]octan-7-yl)-3-pyridyl]azetidin-1-yl]methanone;
trans-[6-(5-Cyclopropyl-4H-1,2,4-triazol-3-yl)-2-azaspiro[3.3]heptan-2-yl]-[3-[4-[3-(difluoromethyl)cyclobutyl]phenyl]azetidin-1-yl]methanone;
cis-[6-(5-Cyclopropyl-4H-1,2,4-triazol-3-yl)-2-azaspiro[3.3]heptan-2-yl]-[3-[4-[3-(difluoromethyl)cyclobutyl]phenyl]azetidin-1-yl]methanone;
[6-(5-Cyclopropyl-4H-1,2,4-triazol-3-yl)-2-azaspiro[3.3]heptan-2-yl]-[3-hydroxy-3-[4-[3-(trifluoromethyl)azetidin-1-yl]phenyl]azetidin-1-yl]methanone;
[6-(5-cyclopropyl-4H-1,2,4-triazol-3-yl)-2-azaspiro[3.3]heptan-2-yl]-[3-[[2-methoxy-4-(trifluoromethyl)phenyl]methoxy]azetidin-1-yl]methanone;
[6-(5-cyclopropyl-4H-1,2,4-triazol-3-yl)-2-azaspiro[3.3]heptan-2-yl]-[3-[[2-methoxy-4-(trifluoromethyl)phenyl]methoxy]azetidin-1-yl]methanone;
[6-(5-cyclopropyl-4H-1,2,4-triazol-3-yl)-2-azaspiro[3.3]heptan-2-yl]-[3-[[2-fluoro-4-(trifluoromethylsulfonyl)phenyl]methoxy]azetidin-1-yl]methanone;
2-cyclopropyl-6-[1-[6-(5-cyclopropyl-4H-1,2,4-triazol-3-yl)-2-azaspiro[3.3]heptane-2-carbonyl]azetidin-3-yl]oxy-benzonitrile;
[6-(5-cyclopropyl-4H-1,2,4-triazol-3-yl)-2-azaspiro[3.3]heptan-2-yl]-[3-[[2-fluoro-4-(trifluoromethyl)phenyl]methoxy]azetidin-1-yl]methanone;
[3-(4-cyclopropylphenoxy)azetidin-1-yl]-[6-[5-(1-hydroxycyclopropyl)-4H-1,2,4-triazol-3-yl]-2-azaspiro[3.3]heptan-2-yl]methanone;
[3-(4-cyclopropyl-2-fluoro-phenoxy)azetidin-1-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[3-[6-(trifluoromethyl)pyridazin-3-yl]oxyazetidin-1-yl]methanone;
[6-(5-cyclopropyl-4H-1,2,4-triazol-3-yl)-2-azaspiro[3.3]heptan-2-yl]-[3-[[4-(trifluoromethoxy)phenyl]methoxy]azetidin-1-yl]methanone;
[6-(5-cyclopropyl-4H-1,2,4-triazol-3-yl)-2-azaspiro[3.3]heptan-2-yl]-[3-[[4-(trifluoromethylsulfonyl)phenyl]methoxy]azetidin-1-yl]methanone;
5-cyclopropyl-2-[1-[6-[5-(1-hydroxycyclopropyl)-4H-1,2,4-triazol-3-yl]-2-azaspiro[3.3]heptane-2-carbonyl]azetidin-3-yl]oxy-benzonitrile;
methyl 2-[3-[1-[6-(5-cyclopropyl-4H-1,2,4-triazol-3-yl)-2-azaspiro[3.3]heptane-2-carbonyl]azetidin-3-yl]oxyphenyl]-2-methyl-propanoate;
[3-[3-cyclopropyl-4-(trifluoromethyl)phenoxy]azetidin-1-yl]-[6-[5-(1-hydroxycyclopropyl)-4H-1,2,4-triazol-3-yl]-2-azaspiro[3.3]heptan-2-yl]methanone;
[3-[(2-chloro-4-fluoro-phenyl)methoxy]azetidin-1-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[3-[[3-(trifluoromethylsulfonyl)phenyl]methoxy]azetidin-1-yl]methanone;
[3-(3-cyclopropyl-2-fluoro-phenoxy)azetidin-1-yl]-[6-[5-(1-hydroxycyclopropyl)-4H-1,2,4-triazol-3-yl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[3-[4-(trifluoromethyl)phenoxy]azetidin-1-yl]methanone;
[3-[[2-fluoro-4-(trifluoromethyl)phenyl]methoxy]azetidin-1-yl]-[6-[5-(1-hydroxycyclopropyl)-4H-1,2,4-triazol-3-yl]-2-azaspiro[3.3]heptan-2-yl]methanone;
[3-(4-chloro-3-cyclopropyl-phenoxy)azetidin-1-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[3-[(4,4-difluorocyclohexyl)methoxy]azetidin-1-yl]methanone;
[6-(5-cyclopropyl-4H-1,2,4-triazol-3-yl)-2-azaspiro[3.3]heptan-2-yl]-[3-[(6,6-difluoro-3-bicyclo[3.1.0]hexanyl)methoxy]azetidin-1-yl]methanone;
[3-[(6-chloro-5-cyclopropyl-3-pyridyl)oxy]azetidin-1-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[3-[[5-(trifluoromethyl)-3-pyridyl]methoxy]azetidin-1-yl]methanone;
[6-(5-cyclopropyl-4H-1,2,4-triazol-3-yl)-2-azaspiro[3.3]heptan-2-yl]-[3-[(2-fluoro-4-methylsulfonyl-phenyl)methoxy]azetidin-1-yl]methanone;
[6-(5-cyclopropyl-4H-1,2,4-triazol-3-yl)-2-azaspiro[3.3]heptan-2-yl]-[3-[[1-(trifluoromethyl)cyclopropyl]methoxy]azetidin-1-yl]methanone;
[6-(5-cyclopropyl-4H-1,2,4-triazol-3-yl)-2-azaspiro[3.3]heptan-2-yl]-[3-[[6-(trifluoromethyl)-3-pyridyl]methoxy]azetidin-1-yl]methanone;
[6-(5-cyclopropyl-4H-1,2,4-triazol-3-yl)-2-azaspiro[3.3]heptan-2-yl]-[3-[(4-methylsulfonylphenyl)methoxy]azetidin-1-yl]methanone;
[6-(5-cyclopropyl-4H-1,2,4-triazol-3-yl)-2-azaspiro[3.3]heptan-2-yl]-[3-[(3-methylsulfonylphenyl)methoxy]azetidin-1-yl]methanone;
[6-(5-cyclopropyl-4H-1,2,4-triazol-3-yl)-2-azaspiro[3.3]heptan-2-yl]-[3-[(2-methylsulfonylphenyl)methoxy]azetidin-1-yl]methanone;
[3-[[2-fluoro-4-(trifluoromethyl)phenyl]methoxy]azetidin-1-yl]-[6-[5-(2,2,2-trifluoro-1-hydroxy-ethyl)-4H-1,2,4-triazol-3-yl]-2-azaspiro[3.3]heptan-2-yl]methanone;
[3-[4-(4-chloro-2-methylsulfonyl-phenyl)phenyl]azetidin-1-yl]-[6-(5-cyclopropyl-4H-1,2,4-triazol-3-yl)-2-azaspiro[3.3]heptan-2-yl]methanone;
[3-[4-(2-chloro-4-methylsulfonyl-phenyl)phenyl]azetidin-1-yl]-[6-(5-cyclopropyl-4H-1,2,4-triazol-3-yl)-2-azaspiro[3.3]heptan-2-yl]methanone;
methyl 2-[4-[1-[6-(5-cyclopropyl-4H-1,2,4-triazol-3-yl)-2-azaspiro[3.3]heptane-2-carbonyl]azetidin-3-yl]phenyl]benzoate;
2-[4-[1-[6-(5-cyclopropyl-4H-1,2,4-triazol-3-yl)-2-azaspiro[3.3]heptane-2-carbonyl]azetidin-3-yl]phenyl]benzamide;
[6-(5-cyclopropyl-4H-1,2,4-triazol-3-yl)-2-azaspiro[3.3]heptan-2-yl]-[3-[3-[[[1-(trifluoromethyl)cyclopropyl]amino]methyl]-1-bicyclo[1.1.1]pentanyl]azetidin-1-yl]methanone;
[6-(5-cyclopropyl-4H-1,2,4-triazol-3-yl)-2-azaspiro[3.3]heptan-2-yl]-[3-[3-[[1-(trifluoromethyl)cyclopropyl]methylamino]-1-bicyclo[1.1.1]pentanyl]azetidin-1-yl]methanone;
[6-(5-cyclopropyl-4H-1,2,4-triazol-3-yl)-2-azaspiro[3.3]heptan-2-yl]-[3-[3-[5-(2,2-dimethylpropyl)-1,3,4-oxadiazol-2-yl]-1-bicyclo[1.1.1]pentanyl]azetidin-1-yl]methanone;
[6-(5-cyclopropyl-4H-1,2,4-triazol-3-yl)-2-azaspiro[3.3]heptan-2-yl]-[3-[3-[5-(2,2,2-trifluoroethyl)-1,3,4-oxadiazol-2-yl]-1-bicyclo[1.1.1]pentanyl]azetidin-1-yl]methanone;
[3-[3-(5-cyclopropyl-1,3,4-oxadiazol-2-yl)-1-bicyclo[1.1.1]pentanyl]azetidin-1-yl]-[6-(5-cyclopropyl-4H-1,2,4-triazol-3-yl)-2-azaspiro[3.3]heptan-2-yl]methanone;
[7-(2,5-dichlorophenoxy)-2-azaspiro[3.5]nonan-2-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[3-[[2-fluoro-4-(trifluoromethyl)phenoxy]methyl]azetidin-1-yl]methanone;
N-[2-[6-(5-cyclopropyl-4H-1,2,4-triazol-3-yl)-2-azaspiro[3.3]heptane-2-carbonyl]-2-azaspiro[3.5]nonan-7-yl]-3-(trifluoromethoxy)benzenesulfonamide;
[3-[2-[2-fluoro-4-(trifluoromethyl)phenyl]ethyl]azetidin-1-yl]-[6-[3-(1-hydroxycyclopropyl)-1H-1,2,4-triazol-5-yl]-2-azaspiro[3.3]heptan-2-yl]methanone;
[7-(5-chloro-2-methylsulfonyl-phenoxy)-2-azaspiro[3.5]nonan-2-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[7-[[5-(trifluoromethyl)pyrazin-2-yl]amino]-2-azaspiro[3.5]nonan-2-yl]methanone;
[6-(5-cyclopropyl-4H-1,2,4-triazol-3-yl)-2-azaspiro[3.3]heptan-2-yl]-[6-[[[1-(trifluoromethyl)cyclopropyl]methylamino]methyl]-2-azaspiro[3.3]heptan-2-yl]methanone;
[3-[(2-chloro-4-fluoro-phenoxy)methyl]azetidin-1-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[6-[[[3-(trifluoromethyl)oxetan-3-yl]amino]methyl]-2-azaspiro[3.3]heptan-2-yl]methanone;
[6-(5-cyclopropyl-4H-1,2,4-triazol-3-yl)-2-azaspiro[3.3]heptan-2-yl]-[7-(4-fluoro-2-methylsulfonyl-phenoxy)-2-azaspiro[3.5]nonan-2-yl]methanone;
[6-(2-chloro-4-fluoro-phenyl)sulfonyl-2-azaspiro[3.3]heptan-2-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[6-[[(3-methyl-1,1-dioxo-thietan-3-yl)amino]methyl]-2-azaspiro[3.3]heptan-2-yl]methanone;
N-[2-[6-(5-cyclopropyl-4H-1,2,4-triazol-3-yl)-2-azaspiro[3.3]heptane-2-carbonyl]-2-azaspiro[3.5]nonan-7-yl]-4,4-di fluoro-cyclohexanesulfonamide;
2-[6-(5-cyclopropyl-4H-1,2,4-triazol-3-yl)-2-azaspiro[3.3]heptane-2-carbonyl]-N-[[1-(trifluoromethyl)cyclopropyl]methyl]-2-azaspiro[3.3]heptane-6-carboxamide;
[6-(5-cyclopropyl-4H-1,2,4-triazol-3-yl)-2-azaspiro[3.3]heptan-2-yl]-[3-[1-[[4-(trifluoromethyl)phenyl]methyl]azetidin-3-yl]azetidin-1-yl]methanone;
[3-[(4-chlorophenyl)methyl]azetidin-1-yl]-[6-[5-(1-hydroxycyclopropyl)-4H-1,2,4-triazol-3-yl]-2-azaspiro[3.3]heptan-2-yl]methanone;
2-[1-[6-(5-cyclopropyl-4H-1,2,4-triazol-3-yl)-2-azaspiro[3.3]heptane-2-carbonyl]-4-piperidyl]-2-(4-fluorophenyl)acetamide;
[(3aR,6aS)-2-benzyl-1,3,3a,4,6,6a-hexahydropyrrolo[3,4-c]pyrrol-5-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[6-[[(1,1-dioxothietan-3-yl)amino]methyl]-2-azaspiro[3.3]heptan-2-yl]methanone;
[3-[4-[1-(2-tert-butyltetrazol-5-yl)cyclopropyl]phenyl]azetidin-1-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[3-[4-(2,2,2-trifluoro-1,1-dimethyl-ethoxy)phenyl]azetidin-1-yl]methanone;
[3-[6-(2-chlorophenoxy)-3-pyridyl]azetidin-1-yl]-[6-(5-cyclopropyl-4H-1,2,4-triazol-3-yl)-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[3-[4-(2,2,2-trifluoro-1,1-dimethyl-ethoxy)phenyl]azetidin-1-yl]methanone;
[6-(5-cyclopropyl-4H-1,2,4-triazol-3-yl)-2-azaspiro[3.3]heptan-2-yl]-[3-[4-[3-(2,2-dimethylpropyl)triazol-4-yl]phenyl]azetidin-1-yl]methanone;
[6-(5-cyclopropyl-4H-1,2,4-triazol-3-yl)-2-azaspiro[3.3]heptan-2-yl]-[3-[4-[5-(trifluoromethyl)pyrazin-2-yl]oxyphenyl]azetidin-1-yl]methanone;
[3-[4-(benzenesulfonyl)phenyl]azetidin-1-yl]-[6-(5-cyclopropyl-4H-1,2,4-triazol-3-yl)-2-azaspiro[3.3]heptan-2-yl]methanone;
methyl 1-[4-[1-[6-(5-cyclopropyl-4H-1,2,4-triazol-3-yl)-2-azaspiro[3.3]heptane-2-carbonyl]azetidin-3-yl]phenyl]cyclopropanecarboxylate;
[3-(4-cyclohexylsulfonylphenyl)azetidin-1-yl]-[6-(5-cyclopropyl-4H-1,2,4-triazol-3-yl)-2-azaspiro[3.3]heptan-2-yl]methanone;
[3-[6-(2-chlorophenoxy)-3-pyridyl]azetidin-1-yl]-[6-[5-(1-hydroxycyclopropyl)-4H-1,2,4-triazol-3-yl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[3-[4-[[1-(trifluoromethyl)cyclopropyl]methoxy]phenyl]azetidin-1-yl]methanone;
1-[4-[1-[6-(5-cyclopropyl-4H-1,2,4-triazol-3-yl)-2-azaspiro[3.3]heptane-2-carbonyl]azetidin-3-yl]phenyl]cyclopropanecarbonitrile;
[6-(3-cyclopropyl-1H-1,2,4-triazol-5-yl)-2-azaspiro[3.3]heptan-2-yl]-[3-[4-[5-[1-(trifluoromethyl)cyclopropyl]-4H-1,2,4-triazol-3-yl]phenyl]azetidin-1-yl]methanone;
[3-[6-(2-chlorophenoxy)pyridazin-3-yl]azetidin-1-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[3-[4-[1-(1H-tetrazol-5-yl)cyclopropyl]phenyl]azetidin-1-yl]methanone;
4-[1-[6-(5-cyclopropyl-4H-1,2,4-triazol-3-yl)-2-azaspiro[3.3]heptane-2-carbonyl]azetidin-3-yl]benzenesulfonamide;
[6-(5-cyclopropyl-4H-1,2,4-triazol-3-yl)-2-azaspiro[3.3]heptan-2-yl]-[3-[4-(dimethylphosphorylmethyl)phenyl]azetidin-1-yl]methanone;
[6-(5-cyclopropyl-4H-1,2,4-triazol-3-yl)-2-azaspiro[3.3]heptan-2-yl]-[3-[6-(4-isopropyl-N-methyl-anilino)-3-pyridyl]azetidin-1-yl]methanone;
[6-(5-cyclopropyl-4H-1,2,4-triazol-3-yl)-2-azaspiro[3.3]heptan-2-yl]-[3-[6-(N-methylanilino)-3-pyridyl]azetidin-1-yl]methanone;
1-[4-[1-[6-(5-cyclopropyl-4H-1,2,4-triazol-3-yl)-2-azaspiro[3.3]heptane-2-carbonyl]azetidin-3-yl]phenyl]piperidine-2-carboxamide;
1-[4-[1-[6-(5-cyclopropyl-4H-1,2,4-triazol-3-yl)-2-azaspiro[3.3]heptane-2-carbonyl]azetidin-3-yl]phenyl]-4,4-difluoro-piperidine-2-carboxamide;
1-[4-[1-[6-(5-cyclopropyl-4H-1,2,4-triazol-3-yl)-2-azaspiro[3.3]heptane-2-carbonyl]azetidin-3-yl]phenyl]pyrrolidine-2-carboxamide;
2-[3-[1-[6-(5-cyclopropyl-4H-1,2,4-triazol-3-yl)-2-azaspiro[3.3]heptane-2-carbonyl]azetidin-3-yl]oxyphenyl]-2-methyl-propanoic acid;
[3-[[2-fluoro-4-(trifluoromethyl)phenyl]methoxy]azetidin-1-yl]-[6-[5-[(1R)-2,2,2-trifluoro-1-hydroxy-ethyl]-4H-1,2,4-triazol-3-yl]-2-azaspiro[3.3]heptan-2-yl]methanone;
[3-[[2-fluoro-4-(trifluoromethyl)phenyl]methoxy]azetidin-1-yl]-[6-[5-[(1S)-2,2,2-trifluoro-1-hydroxy-ethyl]-4H-1,2,4-triazol-3-yl]-2-azaspiro[3.3]heptan-2-yl]methanone;
2-[4-[1-[6-(5-cyclopropyl-4H-1,2,4-triazol-3-yl)-2-azaspiro[3.3]heptane-2-carbonyl]azetidin-3-yl]phenyl]benzoic acid;
2-[4-[1-[6-(5-cyclopropyl-4H-1,2,4-triazol-3-yl)-2-azaspiro[3.3]heptane-2-carbonyl]azetidin-3-yl]phenyl]-N-ethyl-benzamide;
1-[4-[1-[6-(5-cyclopropyl-4H-1,2,4-triazol-3-yl)-2-azaspiro[3.3]heptane-2-carbonyl]azetidin-3-yl]phenyl]cyclopropanecarboxylic acid;
1-[4-[1-[6-(5-cyclopropyl-4H-1,2,4-triazol-3-yl)-2-azaspiro[3.3]heptane-2-carbonyl]azetidin-3-yl]phenyl]cyclopropanecarboxamide;
(2S)-1-[4-[1-[6-(5-cyclopropyl-4H-1,2,4-triazol-3-yl)-2-azaspiro[3.3]heptane-2-carbonyl]azetidin-3-yl]phenyl]piperidine-2-carboxamide;
(2S)-1-[4-[1-[6-(5-cyclopropyl-4H-1,2,4-triazol-3-yl)-2-azaspiro[3.3]heptane-2-carbonyl]azetidin-3-yl]phenyl]-4,4-difluoro-piperidine-2-carboxamide;
(2R)-1-[4-[1-[6-(5-cyclopropyl-4H-1,2,4-triazol-3-yl)-2-azaspiro[3.3]heptane-2-carbonyl]azetidin-3-yl]phenyl]-4,4-difluoro-piperidine-2-carboxamide;
(2S)-1-[4-[1-[6-(5-cyclopropyl-4H-1,2,4-triazol-3-yl)-2-azaspiro[3.3]heptane-2-carbonyl]azetidin-3-yl]phenyl]pyrolidine-2-carboxamide;
(2R)-1-[4-[1-[6-(5-cyclopropyl-4H-1,2,4-triazol-3-yl)-2-azaspiro[3.3]heptane-2-carbonyl]azetidin-3-yl]phenyl]pyrrolidine-2-carboxamide;
[3-(6-tert-butyl-3-pyridyl)azetidin-1-yl]-[6-(3-cyclopropyl-1H-1,2,4-triazol-5-yl)-2-azaspiro[3.3]heptan-2-yl]methanone;
[[6-(5-cyclopropyl-4H-1,2,4-triazol-3-yl)-2-azaspiro[3.3]heptan-2-yl]-[6-[[6-(trifluoromethyl)pyrimidin-4-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-(trifluoromethyl)pyrazol-4-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[2-(trifluoromethyl)pyrimidin-4-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[4-(trifluoromethyl)pyrimidin-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)-1H-pyrazol-5-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-cyclopropyl-3-(trifluoromethyl)pyrazol-4-yl]methyl]-2-azaspiro[3.3]heptan-2-yl]methanone;
1-[[2-[6-(3-cyclopropyl-1H-1,2,4-triazol-5-yl)-2-azaspiro[3.3]heptane-2-carbonyl]-2-azaspiro[3.3]heptan-6-yl]methyl]-5-(trifluoromethyl)-2-pyridone;
bis[6-(3-cyclopropyl-1H-1,2,4-triazol-5-yl)-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[3-[4-[5-methyl-3-(trifluoromethyl)pyrazol-1-yl]phenyl]azetidin-1-yl]methanone;
1-[[2-[6-(3-cyclopropyl-1H-1,2,4-triazol-5-yl)-2-azaspiro[3.3]heptane-2-carbonyl]-2-azaspiro[3.3]heptan-6-ylidene]methyl]-5-(trifluoromethyl)-2-pyridone;
[6-(3-cyclopropyl-1H-1,2,4-triazol-5-yl)-2-azaspiro[3.3]heptan-2-yl]-[6-[[5-(trifluoromethyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[4-(trifluoromethyl)triazol-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[4-(trifluoromethyl)triazol-1-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-(4-triflyphenyl)-2-azaspiro[3.3]heptan-2-yl]methanone;
[2-(4-chloro-2-mesyl-benzyl)-6-azaspiro[3.4]octan-6-yl]-[6-(5-cyclopropyl-4H-1,2,4-triazol-3-yl)-2-azaspiro[3.3]heptan-2-yl]methanone;
[6-[(2-tert-butylthiazol-5-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[4-[5-chloro-1-(2-hydroxy-2-methyl-propyl)indazol-3-yl]piperidino]-[6-(5-cyclopropyl-4H-1,2,4-triazol-3-yl)-2-azaspiro[3.3]heptan-2-yl]methanone;
1-[[2-[6-[3-(1-hydroxycyclopropyl)-1H-1,2,4-triazol-5-yl]-2-azaspiro[3.3]heptane-2-carbonyl]-2-azaspiro[3.3]heptan-6-yl]methyl]-5-(trifluoromethyl)-2-pyridone;
[6-(3-cyclopropyl-1H-1,2,4-triazol-5-yl)-2-azaspiro[3.3]heptan-2-yl]-[4-[3-[5-[1-(trifluoromethyl)cyclopropyl]-1,3,4-oxadiazol-2-yl]-1-bicyclo[1.1.1]pentanyl]piperidino]methanone;
[6-[3-(1-hydroxycyclopropyl)-1H-1,2,4-triazol-5-yl]-2-azaspiro[3.3]heptan-2-yl]-[3-[3-(4-triflylphenyl)-1-bicyclo[1.1.1]pentanyl]azetidin-1-yl]methanone;
[6-[3-cyclopropyl-1H-1,2,4-triazol-5-yl)-2-azaspiro[3.3]heptan-2-yl]-[6-(4-triflylbenzyl)-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[7-[3-(trifluoromethyl)phenyl]sulfonyl-2-azaspiro[3.5]nonan-2-yl]methanone;
[6-(3-cyclopropyl-1H-1,2,4-triazol-5-yl)-2-azaspiro[3.3]heptan-2-yl]-[3-[3-[5-[1-(trifluoromethyl)cyclopropyl]-1,3,4-oxadiazol-2-yl]-1-bicyclo[1.1.1]pentanyl]azetidin-1-yl]methanone;
[6-(3-cyclopropyl-1H-1,2,4-triazol-5-yl)-2-azaspiro[3.3]heptan-2-yl]-[6-[4-(trifluoromethyl)thiazol-2-yl]oxy-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[5-(trifluoromethyl)pyrazin-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[1-methyl-3-(trifluoromethyl)pyrazol-4-yl]oxy-2-azaspiro[3.3]heptan-2-yl]methanone;
4-[[2-[6-(3-cyclopropyl-1H-1,2,4-triazol-5-yl)-2-azaspiro[3.3]heptane-2-carbonyl]-2,6-diazaspiro[3.3]heptan-6-yl]methyl]-1-(2,2,2-trifluoroethyl)-2-pyridone;
[6-[3-(1-fluorocyclopropyl)-1H-1,2,4-triazol-5-yl]-2-azaspiro[3.3]heptan-2-yl]-[6-(4-triflylbenzyl)-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[3-methyl-5-(trifluoromethyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[7-[[4-(trifluoromethyl)phenyl]sulfonimidoyl]-2-azaspiro[3.5]nonan-2-yl]methanone;
[6-(3-cyclopropyl-1H-1,2,4-triazol-5-yl)-2-azaspiro[3.3]heptan-2-yl]-[6-[[4-(trifluoromethyl)thiazol-2-yl]methyl]-2,6-diazaspiro[3.3]heptan-2-yl]methanone;
[6-[3-(1-fluorocyclopropyl)-1H-1,2,4-triazol-5-yl]-2-azaspiro[3.3]heptan-2-yl]-[3-[5-[[1-(trifluoromethyl)cyclopropyl]methoxy]pyrazin-2-yl]azetidin-1-yl]methanone;
[6-(3-cyclopropyl-1H-1,2,4-triazol-5-yl)-2-azaspiro[3.3]heptan-2-yl]-[7-[[5-(trifluoromethyl)isothiazol-3-yl]methyl]-2,7-diazaspiro[3.5]nonan-2-yl]methanone;
5-[[2-[6-[5-(1-hydroxycyclopropyl)-4H-1,2,4-triazol-3-yl]-2-azaspiro[3.3]heptane-2-carbonyl]-2-azaspiro[3.3]heptan-6-yl]methyl]-2-(trifluoromethyl)isonicotinonitrile;
[6-(3-cyclopropyl-1H-1,2,4-triazol-5-yl)-2-azaspiro[3.3]heptan-2-yl]-[6-[(5-fluoro-2-pyridyl)oxy]-2-azaspiro[3.3]heptan-2-yl]methanone;
[6-(3-cyclopropyl-1H-1,2,4-triazol-5-yl)-2-azaspiro[3.3]heptan-2-yl]-[7-[3-(trifluoromethyl)phenyl]sulfonyl-2-azaspiro[3.5]nonan-2-yl]methanone;
[6-(3-cyclopropyl-1H-1,2,4-triazol-5-yl)-2-azaspiro[3.3]heptan-2-yl]-[7-[[5-(trifluoromethyl)-4H-1,2,4-triazol-3-yl]methyl]-2-azaspiro[3.5]nonan-2-yl]methanone;
[6-(3-cyclopropyl-1H-1,2,4-triazol-5-yl)-2-azaspiro[3.3]heptan-2-yl]-[6-[[I-methyl-5-(trifluoromethyl)triazol-4-yl]methyl]-2,6-diazaspiro[3.3]heptan-2-yl]methanone;
[3-[3-[[2-fluoro-4-(trifluoromethyl)phenyl]methyl]-1-bicyclo[1.1.1]pentanyl]azetidin-1-yl]-[6-[3-(1-hydroxycyclopropyl)-1H-1,2,4-triazol-5-yl]-2-azaspiro[3.3]heptan-2-yl]methanone;
[6-(3-cyclopropyl-1H-1,2,4-triazol-5-yl)-2-azaspiro[3.3]heptan-2-yl]-[4-[3-(5-neopentyl-1,3,4-oxadiazol-2-yl)-1-bicyclo[1.1.1]pentanyl]piperidino]methanone;
[6-(3-cyclopropyl-1H-1,2,4-triazol-5-yl)-2-azaspiro[3.3]heptan-2-yl]-[6-[[3-methyl-1-(2,2,2-trifluoroethyl)pyrazol-4-yl]methyl]-2,6-diazaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[3-(trifluoromethyl)-1,2,4-triazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-(2,2,2-trifluoroethyl)triazol-4-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[3-[5-[[[1-(trifluoromethyl); cyclopropyl]amino]methyl]pyrazin-2-yl]azetidin-1-yl]methanone;
[6-(3-cyclopropyl-1H-1,2,4-triazol-5-yl)-2-azaspiro[3.3]heptan-2-yl]-[6-[[5-(trifluoromethyl)-1,2,4-triazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-methyl-3-(trifluoromethyl)pyrazol-1-yl]methyl]-2-azaspiro[3.3]heptan-2-yl]methanone;
[6-[3-cyclopropyl-1H-1,2,4-triazol-5-yl)-2-azaspiro[3.3]heptan-2-yl]-[6-[difluoro-[6-(trifluoromethyl)pyridazin-3-yl]methyl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[3-[3-[5-[1-(trifluoromethyl)cyclopropyl]-1,3,4-thiadiazol-2-yl]-1-bicyclo[1.1.1]pentanyl]azetidin-1-yl]methanone;
[6-[difluoro-[6-(trifluoromethyl)pyridazin-3-yl]methyl]-2-azaspiro[3.3]heptan-2-yl]-[6-[3-(1-hydroxycyclopropyl)-1H-1,2,4-triazol-5-yl]-2-azaspiro[3.3]heptan-2-yl]methanone;
[6-(5-cyclopropyl-4H-1,2,4-triazol-3-yl)-2-azaspiro[3.3]heptan-2-yl]-[3-[4-(5-ethoxy-3-methyl-pyrazol-1-yl)phenyl]azetidin-1-yl]methanone;
[6-(3-cyclopropyl-1H-1,2,4-triazol-5-yl)-2-azaspiro[3.3]heptan-2-yl]-[6-[[5-(trifluoromethyl)-1,2,4-thiadiazol-3-yl]methyl]-2-azaspiro[3.3]heptan-2-yl]methanone;
[6-(2-fluoro-4-triflyl-benzyl)-2,6-diazaspiro[3.3]heptan-2-yl]-[6-[3-(1-hydroxycyclopropyl)-1H-1,2,4-triazol-5-yl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[6-(trifluoromethyl)pyridazin-3-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[2-(trifluoromethyl)imidazol-1-yl]methyl]-2-azaspiro[3.3]heptan-2-yl]methanone;
[3-[4-(4-chloro-2-mesyl-phenyl)phenyl]azetidin-1-yl]-[6-(1H-1,2,4-triazol-5-yl)-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[3-fluoro-5-(trifluoromethyl)benzyl]-2-azaspiro[3.3]heptan-2-yl]methanone;
[6-(3-cyclopropyl-1H-1,2,4-triazol-5-yl)-2-azaspiro[3.3]heptan-2-yl]-[7-[[3-(trifluoromethyl)phenyl]sulfonimidoyl]-2-azaspiro[3.5]nonan-2-yl]methanone;
[6-[3-(1-fluorocyclopropyl)-1H-1,2,4-triazol-5-yl]-2-azaspiro[3.3]heptan-2-yl]-[6-[[5-(trifluoromethyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-methyl-4-(trifluoromethyl)thiazol-2-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[3-[3-[3-(trifluoromethoxy)phenyl]-1-bicyclo[1.1.1]pentanyl]azetidin-1-yl]methanone;
[6-[3-(1-fluorocyclopropyl)-1H-1,2,4-triazol-5-yl]-2-azaspiro[3.3]heptan-2-yl]-[6-[[6-(trifluoromethyl)pyridazin-3-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[7-[[2-methyl-4-(trifluoromethyl)pyrazol-3-yl]methyl]-2,7-diazaspiro[3.5]nonan-2-yl]methanone;
[6-[3-cyclopropyl-1H-1,2,4-triazol-5-yl)-2-azaspiro[3.3]heptan-2-yl]-[6-(4-dimethylphosphoryl-2-fluoro-benzyl)-2-azaspiro[3.3]heptan-2-yl]methanone;
2-[3-[1-[6-(3-cyclopropyl-1H-1,2,4-triazol-5-yl)-2-azaspiro[3.3]heptane-2-carbonyl]azetidin-3-yl]-1-bicyclo[1.1.1]pentanyl]-5-fluoro-benzonitrile;
3-[[2-[6-[3-(1-fluorocyclopropyl)-1H-1,2,4-triazol-5-yl]-2-azaspiro[3.3]heptane-2-carbonyl]-2-azaspiro[3.3]heptan-6-yl]methyl]-5-(trifluoromethyl)benzonitrile;
[6-[3-(3,3-difluorocyclobutyl)-1H-1,2,4-triazol-5-yl]-2-azaspiro[3.3]heptan-2-yl]-[6-[[5-(trifluoromethyl)pyrazin-2-yl]methyl]-2-azaspiro[3.3]heptan-2-yl]methanone;
[3-[3-[5-(2,2-dimethylpropyl)-1,3,4-thiadiazol-2-yl]-1-bicyclo[1.1.1];
pentanyl]azetidin-1-yl]-[6-[3-(1-hydroxycyclopropyl)-1H-1,2,4-triazol-5-yl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[2-fluoro-4-(trifluoromethyl)benzyl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[4-fluoro-2-(trifluoromethyl)benzyl]-2-azaspiro[3.3]heptan-2-yl]methanone;
[6-(3-cyclopropyl-1H-1,2,4-triazol-5-yl)-2-azaspiro[3.3]heptan-2-yl]-[3-[5-[[1-(trifluoromethyl)cyclopropyl]methoxy]pyrazin-2-yl]azetidin-1-yl]methanone;
[6-[3-(1-fluorocyclopropyl)-1H-1,2,4-triazol-5-yl]-2-azaspiro[3.3]heptan-2-yl]-[3-(2-fluoro-4-triflyl-benzyl)oxyazetidin-1-yl]methanone;
[6-[3-(1-hydroxycyclopropyl)-1H-1,2,4-triazol-5-yl]-2-azaspiro[3.3]heptan-2-yl]-[3-[5-[1-(trifluoromethyl)cyclopropyl]-2-pyridyl]azetidin-1-yl]methanone;
[6-(3-cyclopropyl-1H-1,2,4-triazol-5-yl)-2-azaspiro[3.3]heptan-2-yl]-[6-[[1-methyl-3-(trifluoromethyl)pyrazol-4-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[1-methyl-3-(trifluoromethyl)pyrazol-4-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[3-[[5-(trifluoromethyl)pyrazin-2-yl]amino]-1-bicyclo[1.1.1]pentanyl]methanone;
[6-[3-(1-aminocyclopropyl)-1H-1,2,4-triazol-5-yl]-2-azaspiro[3.3]heptan-2-yl]-[6-[[4-(trifluoromethyl-sulfonimidoyl)phenyl]methyl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[1-methyl-5-(trifluoromethyl)pyrazol-4-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[3-[(8S)-8-methyl-8-oxo-8λ⁶-thia-9-azatricyclo[8.4.0.02,7]tetradeca-1(10),2(7),3,5,8,11,13-heptaen-12-yl]azetidin-1-yl]methanone;
2-[[2-[6-[5-(1-hydroxycyclopropyl)-4H-1,2,4-triazol-3-yl]-2-azaspiro[3.3]heptane-2-carbonyl]-2-azaspiro[3.3]heptan-6-yl]methyl]-5-(trifluoromethyl)benzonitrile;
[6-(5-cyclopropyl-4H-1,2,4-triazol-3-yl)-2-azaspiro[3.3]heptan-2-yl]-[3-[(8R)-8-methyl-8-oxo-8λ⁶-thia-9-azatricyclo[8.4.0.02,7]tetradeca-1(10),2(7),3,5,8,11,13-heptaen-12-yl]azetidin-1-yl]methanone;
[6-(3-cyclopropyl-1H-1,2,4-triazol-5-yl)-2-azaspiro[3.3]heptan-2-yl]-[6-[[1-methyl-4-(trifluoromethyl)pyrazol-3-yl]methyl]-2-azaspiro[3.3]heptan-2-yl]methanone;
1-[[2-[6-(3-cyclopropyl-1H-1,2,4-triazol-5-yl)-2-azaspiro[3.3]heptane-2-carbonyl]-2-azaspiro[3.3]heptan-6-yl]methyl]-6-keto-nicotinonitrile;
[6-(3-cyclopropyl-1H-1,2,4-triazol-5-yl)-2-azaspiro[3.3]heptan-2-yl]-[6-[2-(methylsulfonimidoyl)-4-(trifluoromethyl)benzyl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[7-(3-triflylbenzyl)-2,7-diazaspiro[3.4]octan-2-yl]methanone;
[6-[(4-tert-butyloxazol-2-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[3-(trifluoromethoxy)phenyl]sulfonyl-2-azaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethyl)-1,3,4-thiadiazol-2-yl]methyl]-2,6-diazaspiro[3.3]heptan-2-yl]methanone;
4-[[2-[6-[3-(3,3-difluorocyclobutyl)-1H-1,2,4-triazol-5-yl]-2-azaspiro[3.3]heptane-2-carbonyl]-2-azaspiro[3.3]heptan-6-yl]methyl]-3-fluoro-benzonitrile;
[6-[(5-tert-butyl-1,3,4-thiadiazol-2-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-[(4-cyclopropylthiadiazol-5-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[4-(trifluoromethyl)thiazol-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[3-[3-[4-(trifluoromethoxy)phenyl]-1-bicyclo[1.1.1]pentanyl]azetidin-1-yl]methanone;
[6-[3-(1-hydroxycyclopropyl)-1H-1,2,4-triazol-5-yl]-2-azaspiro[3.3]heptan-2-yl]-[6-[[1-methyl-5-(trifluoromethyl)pyrazol-3-yl]methyl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[3-[3-(5-neopentyl-1,3,4-thiadiazol-2-yl)-1-bicyclo[1.1.1]pentanyl]azetidin-1-yl]methanone;
[6-[3-(3,3-difluorocyclobutyl)-1H-1,2,4-triazol-5-yl]-2-azaspiro[3.3]heptan-2-yl]-[6-[3-(methylsulfonimidoyl)benzyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethyl)-1H-1,2,4-triazol-3-yl]methyl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[5-(difluoromethyl)-1H-pyrazol-3-yl]methyl]-2-azaspiro[3.3]heptan-2-yl]methanone;
[3-[4-(cyclopropylsulfonimidoyl);
phenyl]azetidin-1-yl]-[6-[3-(1-hydroxycyclopropyl)-1H-1,2,4-triazol-5-yl]-2-azaspiro[3.3]heptan-2-yl]methanone;
[6-(5-cyclopropyl-4H-1,2,4-triazol-3-yl)-2-azaspiro[3.3]heptan-2-yl]-[6-(5-fluoro-3-pyridyl)-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[4-[3-(5-neopentyl-1,3,4-oxadiazol-2-yl)-1-bicyclo[1.1.1]pentanyl]piperidino]methanone;
5-[[2-[6-(3-cyclopropyl-1H-1,2,4-triazol-5-yl)-2-azaspiro[3.3]heptane-2-carbonyl]-2-azaspiro[3.3]heptan-6-yl]oxy]-2-(trifluoromethyl)benzonitrile;
[6-(5-cyclopropyl-4H-1,2,4-triazol-3-yl)-2-azaspiro[3.3]heptan-2-yl]-[3-[4-(4-mesylpyrazol-1-yl)phenyl]azetidin-1-yl]methanone;
[6-(3-cyclopropyl-1H-1,2,4-triazol-5-yl)-2-azaspiro[3.3]heptan-2-yl]-[6-[[6-(trifluoromethoxy)-3-pyridyl]methyl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[3-(2-fluoro-4-triflyl-benzyl)oxyazetidin-1-yl]methanone;
[6-[3-(1-fluorocyclopropyl)-1H-1,2,4-triazol-5-yl]-2-azaspiro[3.3]heptan-2-yl]-[3-[5-[[1-(trifluoromethyl);
cyclopropyl]methylamino]pyrazin-2-yl]azetidin-1-yl]methanone;
[6-(3-cyclopropyl-1H-1,2,4-triazol-5-yl)-2-azaspiro[3.3]heptan-2-yl]-[6-[[2-methyl-4-(trifluoromethyl)thiazol-5-yl]methyl]-2,6-diazaspiro[3.3]heptan-2-yl]methanone;
[6-(4-cyclopropylsulfonylbenzyl)-2-azaspiro[3.3]heptan-2-yl]-[6-[3-(1-hydroxycyclopropyl)-1H-1,2,4-triazol-5-yl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-methyl-5-(trifluoromethyl)pyrazol-3-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[7-[[5-(difluoromethyl)thiazol-2-yl]methyl]-2,7-diazaspiro[3.5]nonan-2-yl]methanone;
[6-[3-(3,3-difluorocyclobutyl)-1H-1,2,4-triazol-5-yl]-2-azaspiro[3.3]heptan-2-yl]-[6-[[3-fluoro-5-(trifluoromethyl)-2-pyridyl]methyl]-2-azaspiro[3.3]heptan-2-yl]methanone;
5-fluoro-2-[3-[1-[6-[3-(1-hydroxycyclopropyl)-1H-1,2,4-triazol-5-yl]-2-azaspiro[3.3]heptane-2-carbonyl]azetidin-3-yl]-1-bicyclo[1.1.1]pentanyl]benzonitrile;
1-[[2-[6-(3-cyclopropyl-1H-1,2,4-triazol-5-yl)-2-azaspiro[3.3]heptane-2-carbonyl]-2-azaspiro[3.3]heptan-6-yl]methyl]-2-keto-nicotinonitrile;
[6-[3-(3,3-difluorocyclobutyl)-1H-1,2,4-triazol-5-yl]-2-azaspiro[3.3]heptan-2-yl]-[6-[[1-methyl-5-(trifluoromethyl)pyrazol-3-yl]methyl]-2-azaspiro[3.3]heptan-2-yl]methanone;
5-[[2-[6-[3-(1-fluorocyclopropyl)-1H-1,2,4-triazol-5-yl]-2-azaspiro[3.3]heptane-2-carbonyl]-2-azaspiro[3.3]heptan-6-yl]methyl]-3-(trifluoromethyl)picolinonitrile;
[6-(3-cyclopropyl-1H-1,2,4-triazol-5-yl)-2-azaspiro[3.3]heptan-2-yl]-[6-[[1-(trifluoromethyl)pyrazol-4-yl]methyl]-2,6-diazaspiro[3.3]heptan-2-yl]methanone;
[3-[3-(3-cyclopropyl-1H-pyrazol-5-yl)-1-bicyclo[1.1.1]pentanyl]azetidin-1-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethyl)-1H-pyrazol-4-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[3-[4-(2-mesylphenyl)phenyl]azetidin-1-yl]methanone;
[6-[3-(1-fluorocyclopropyl)-1H-1,2,4-triazol-5-yl]-2-azaspiro[3.3]heptan-2-yl]-[6-[[5-(trifluoromethyl)-2-pyridyl]methyl]-2-azaspiro[3.3]heptan-2-yl]methanone;
[6-(3-fluoro-5-triflyl-benzyl)-2-azaspiro[3.3]heptan-2-yl]-[6-[3-(1-hydroxycyclopropyl)-1H-1,2,4-triazol-5-yl]-2-azaspiro[3.3]heptan-2-yl]methanone;
[6-(5-cyclopropyl-4H-1,2,4-triazol-3-yl)-2-azaspiro[3.3]heptan-2-yl]-[4-[1-(2-hydroxyethyl)-5-(trifluoromethyl)indazol-3-yl]piperidino]methanone:
[6-(3-cyclopropyl-1H-1,2,4-triazol-5-yl)-2-azaspiro[3.3]heptan-2-yl]-[6-[[5-(trifluoromethyl)-3-pyridyl]oxy]-2-azaspiro[3.3]heptan-2-yl]methanone; [6-(3-cyclopropyl-1H-1,2,4-triazol-5-yl)-2-azaspiro[3.3]heptan-2-yl]-[6-[[1-(2,2,2-trifluoroethyl)tetrazol-5-yl]methyl]-2,6-diazaspiro[3.3]heptan-2-yl]methanone;
[6-[3-(1-fluorocyclopropyl)-1H-1,2,4-triazol-5-yl]-2-azaspiro[3.3]heptan-2-yl]-[6-[2-fluoro-4-(trifluoromethyl)benzyl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[4-(trifluoromethyl)pyrazol-1-yl]methyl]-2-azaspiro[3.3]heptan-2-yl]methanone;
[6-[3-cyclopropyl-1H-1,2,4-triazol-5-yl)-2-azaspiro[3.3]heptan-2-yl]-[6-[[4-(trifluoromethyl)-1H-imidazol-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[7-[[5-(trifluoromethyl)-1H-pyrazol-3-yl]methyl]-2,7-diazaspiro[3.5]nonan-2-yl]methanone;
[6-[3-(3,3-difluorocyclobutyl)-1H-1,2,4-triazol-5-yl]-2-azaspiro[3.3]heptan-2-yl]-[6-(4-triflylbenzyl)-2-azaspiro[3.3]heptan-2-yl]methanone;
[6-(3-cyclopropyl-1H-1,2,4-triazol-5-yl)-2-azaspiro[3.3]heptan-2-yl]-[3-[3-[[2-fluoro-4-(trifluoromethyl);
phenyl]methyl]-1-bicyclo[1.1.1];
pentanyl]azetidin-1-yl]methanone;
[6-(3-cyclopropyl-1H-1,2,4-triazol-5-yl)-2-azaspiro[3.3]heptan-2-yl]-[3-[3-[[1-(trifluoromethyl)cyclopropyl];
methylsulfonyl]-1-bicyclo[1.1.1];
pentanyl]azetidin-1-yl]methanone;
[6-[3-(1-hydroxycyclopropyl)-1H-1,2,4-triazol-5-yl]-2-azaspiro[3.3]heptan-2-yl]-[3-[3-[3-(trifluoromethoxy)phenyl]-1-bicyclo[1.1.1]pentanyl]azetidin-1-yl]methanone;
[6-[3-(3,3-difluorocyclobutyl)-1H-1,2,4-triazol-5-yl]-2-azaspiro[3.3]heptan-2-yl]-[6-[[I-methyl-5-(trifluoromethyl)pyrazol-4-yl]methyl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[3-(trifluoromethyl)isothiazol-5-yl]methyl]-2,6-diazaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[3-(trifluoromethylsulfonimidoyl)benzyl]-2-azaspiro[3.3]heptan-2-yl]methanone;
[6-(3-cyclopropyl-1H-1,2,4-triazol-5-yl)-2-azaspiro[3.3]heptan-2-yl]-[6-[3-(trifluoromethoxy)phenyl]sulfonyl-2-azaspiro[3.3]heptan-2-yl]methanone;
[6-(5-cyclopropyl-4H-1,2,4-triazol-3-yl)-2-azaspiro[3.3]heptan-2-yl]-[3-[4-[3-(difluoromethyl)-5-methyl-1,2,4-triazol-1-yl]phenyl]azetidin-1-yl]methanone;
5-[[2-[6-(5-cyclopropyl-4H-1,2,4-triazol-3-yl)-2-azaspiro[3.3]heptane-2-carbonyl]-2-azaspiro[3.3]heptan-6-yl]methyl]-3-(trifluoromethyl)picolinonitrile;
[6-[3-(1-hydroxycyclopropyl)-1H-1,2,4-triazol-5-yl]-2-azaspiro[3.3]heptan-2-yl]-[6-[[4-(trifluoromethyl)thiazol-2-yl]methyl]-2-azaspiro[3.3]heptan-2-yl]methanone;
5-[[2-[6-(3-cyclopropyl-1H-1,2,4-triazol-5-yl)-2-azaspiro[3.3]heptane-2-carbonyl]-2,6-diazaspiro[3.3]heptan-6-yl]methyl]-1-methyl-imidazole-4-carbonitrile;
[2-(4-chloro-2-mesyl-phenyl)-6-azaspiro[3.4]octan-6-yl]-[6-(5-cyclopropyl-4H-1,2,4-triazol-3-yl)-2-azaspiro[3.3]heptan-2-yl]methanone;
[6-(3-cyclopropyl-1H-1,2,4-triazol-5-yl)-2-azaspiro[3.3]heptan-2-yl]-[7-[[2-methyl-5-(trifluoromethyl)pyrazol-3-yl]methyl]-2,7-diazaspiro[3.5]nonan-2-yl]methanone;
[6-(3-cyclopropyl-1H-1,2,4-triazol-5-yl)-2-azaspiro[3.3]heptan-2-yl]-[3-[3-(4-triflylphenyl)-1-bicyclo[1.1.1]pentanyl]azetidin-1-yl]methanone;
5-[[2-[6-(3-cyclopropyl-1H-1,2,4-triazol-5-yl)-2-azaspiro[3.3]heptane-2-carbonyl]-2,6-diazaspiro[3.3]heptan-6-yl]methyl]-2-pyridone;
[6-(3-cyclopropyl-1H-1,2,4-triazol-5-yl)-2-azaspiro[3.3]heptan-2-yl]-[6-(1-methylpyrazol-4-yl)oxy-2-azaspiro[3.3]heptan-2-yl]methanone;
[6-(3-cyclopropyl-1H-1,2,4-triazol-5-yl)-2-azaspiro[3.3]heptan-2-yl]-[(6S)-6-(3-triflylbenzyl)-2-azaspiro[3.4]octan-2-yl]methanone;
[6-(3-cyclopropyl-1H-1,2,4-triazol-5-yl)-2-azaspiro[3.3]heptan-2-yl]-[7-[[5-(trifluoromethyl)isoxazol-3-yl]methyl]-2,7-diazaspiro[3.5]nonan-2-yl]methanone;
[6-[3-(3,3-difluorocyclobutyl)-1H-1,2,4-triazol-5-yl]-2-azaspiro[3.3]heptan-2-yl]-[6-[[5-(trifluoromethyl)-3-pyridyl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[2-(2,2,2-trifluoroethyl)tetrazol-5-yl]methyl]-2,6-diazaspiro[3.3]heptan-2-yl]methanone;
5-[[2-[6-[3-(3,3-difluorocyclobutyl)-1H-1,2,4-triazol-5-yl]-2-azaspiro[3.3]heptane-2-carbonyl]-2-azaspiro[3.3]heptan-6-yl]methyl]-2-(trifluoromethyl)benzonitrile;
[6-[3-(1-fluorocyclopropyl)-1H-1,2,4-triazol-5-yl]-2-azaspiro[3.3]heptan-2-yl]-[6-[[4-(trifluoromethyl)triazol-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[7-(3-triflylbenzyl)-2,7-diazaspiro[3.4]octan-2-yl]methanone;
[6-[3-(1-hydroxycyclopropyl)-1H-1,2,4-triazol-5-yl]-2-azaspiro[3.3]heptan-2-yl]-[7-[4-(trifluoromethyl)phenyl]sulfonyl-2-azaspiro[3.5]nonan-2-yl]methanone;
[6-[3-(1-hydroxycyclopropyl)-1H-1,2,4-triazol-5-yl]-2-azaspiro[3.3]heptan-2-yl]-[3-[3-[4-(trifluoromethoxy)phenyl]-1-bicyclo[1.1.1]pentanyl]azetidin-1-yl]methanone;
[6-[3-(1-hydroxycyclopropyl)-1H-1,2,4-triazol-5-yl]-2-azaspiro[3.3]heptan-2-yl]-[3-[3-[5-(trifluoromethyl)pyrazin-2-yl]oxy-1-bicyclo[1.1.1]pentanyl]azetidin-1-yl]methanone;
[6-(3-cyclopropyl-1H-1,2,4-triazol-5-yl)-2-azaspiro[3.3]heptan-2-yl]-[6-[[2-ethyl-5-(trifluoromethyl)pyrazol-3-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-(2-fluoro-4-triflyl-benzyl)-2,6-diazaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[4-(trifluoromethylsulfonimidoyl)benzyl]-2-azaspiro[3.3]heptan-2-yl]methanone;
2-[[2-[6-(5-cyclopropyl-4H-1,2,4-triazol-3-yl)-2-azaspiro[3.3]heptane-2-carbonyl]-2-azaspiro[3.3]heptan-6-yl]methyl]-5-(trifluoromethyl)benzonitrile;
[6-(3-cyclopropyl-1H-1,2,4-triazol-5-yl)-2-azaspiro[3.3]heptan-2-yl]-[3-[3-[5-[1-(trifluoromethyl)cyclopropyl]-1,3,4-thiadiazol-2-yl]-1-bicyclo[1.1.1]pentanyl]azetidin-1-yl]methanone;
[6-[3-(1-hydroxycyclopropyl)-1H-1,2,4-triazol-5-yl]-2-azaspiro[3.3]heptan-2-yl]-[6-[4-(trifluoromethylsulfonimidoyl)benzyl]-2-azaspiro[3.3]heptan-2-yl]methanone;
[6-(5-cyclopropyl-4H-1,2,4-triazol-3-yl)-2-azaspiro[3.3]heptan-2-yl]-[3-[4-(3-ethoxy-5-methyl-pyrazol-1-yl)phenyl]azetidin-1-yl]methanone;
[6-[3-(1-hydroxycyclopropyl)-1H-1,2,4-triazol-5-yl]-2-azaspiro[3.3]heptan-2-yl]-[6-[(5-triflyl-3-pyridyl)methyl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[3-(trifluoromethyl)isothiazol-5-yl]methyl]-2,6-diazaspiro[3.3]heptan-2-yl]methanone;
[3-[3-(4-chloro-2-mesyl-phenyl)-1-bicyclo[1.1.1]pentanyl]azetidin-1-yl]-[6-(5-cyclopropyl-4H-1,2,4-triazol-3-yl)-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[3-[4-[1-(trifluoromethyl)cyclopropyl]phenyl]azetidin-1-yl]methanone;
[6-[3-(1-fluorocyclopropyl)-1H-1,2,4-triazol-5-yl]-2-azaspiro[3.3]heptan-2-yl]-[3-[4-(trifluoromethylsulfonimidoyl)phenyl]azetidin-1-yl]methanone;
[6-[3-(1-fluorocyclopropyl)-1H-1,2,4-triazol-5-yl]-2-azaspiro[3.3]heptan-2-yl]-[6-[[2-(trifluoromethyl)pyrimidin-5-yl]methyl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[6-(trifluoromethoxy)-3-pyridyl]methyl]-2-azaspiro[3.3]heptan-2-yl]methanone;
1-[[2-[6-[3-(3,3-difluorocyclobutyl)-1H-1,2,4-triazol-5-yl]-2-azaspiro[3.3]heptane-2-carbonyl]-2-azaspiro[3.3]heptan-6-yl]methyl]pyrazole-4-carbonitrile;
[6-(3-cyclopropyl-1H-1,2,4-triazol-5-yl)-2-azaspiro[3.3]heptan-2-yl]-[6-[[1-(2,2,2-trifluoroethyl)pyrazol-4-yl]methyl]-2,6-diazaspiro[3.3]heptan-2-yl]methanone;
4-[[2-[6-[3-(1-fluorocyclopropyl)-1H-1,2,4-triazol-5-yl]-2-azaspiro[3.3]heptane-2-carbonyl]-2-azaspiro[3.3]heptan-6-yl]methyl]-2-(trifluoromethyl)benzonitrile;
[6-[3-(1-hydroxycyclopropyl)-1H-1,2,4-triazol-5-yl]-2-azaspiro[3.3]heptan-2-yl]-[3-[3-[[6-(trifluoromethyl)-3-pyridyl]methyl]-1-bicyclo[1.1.1]pentanyl]azetidin-1-yl]methanone;
[6-(3-cyclopropyl-1H-1,2,4-triazol-5-yl)-2-azaspiro[3.3]heptan-2-yl]-[6-[[1-(2,2,2-trifluoroethyl)-3-(trifluoromethyl)pyrazol-4-yl]methyl]-2-azaspiro[3.3]heptan-2-yl]methanone;
3-[[2-[6-(3-cyclopropyl-1H-1,2,4-triazol-5-yl)-2-azaspiro[3.3]heptane-2-carbonyl]-2,6-diazaspiro[3.3]heptan-6-yl]methyl]-1-(2,2,2-trifluoroethyl)-2-pyridone;
[6-[3-(1-hydroxycyclopropyl)-1H-1,2,4-triazol-5-yl]-2-azaspiro[3.3]heptan-2-yl]-[3-[3-[5-[1-(trifluoromethyl)cyclopropyl]-1,3,4-oxadiazol-2-yl]-1-bicyclo[1.1.1]pentanyl]azetidin-1-yl]methanone;
[6-[3-(3,3-difluorocyclobutyl)-1H-1,2,4-triazol-5-yl]-2-azaspiro[3.3]heptan-2-yl]-[7-[[5-(trifluoromethyl)-2-pyridyl]methyl]-2-azaspiro[3.5]nonan-2-yl]methanone;
[6-(5-cyclopropyl-4H-1,2,4-triazol-3-yl)-2-azaspiro[3.3]heptan-2-yl]-[6-[[3-(trifluoromethoxy)phenyl]sulfonimidoyl]-2-azaspiro[3.3]heptan-2-yl]methanone;
[3-[2-fluoro-4-(trifluoromethyl-sulfonimidoyl)benzyl]oxyazetidin-1-yl]-[6-[3-(1-hydroxycyclopropyl)-1H-1,2,4-triazol-5-yl]-2-azaspiro[3.3]heptan-2-yl]methanone;
[2-(4-chloro-2-mesyl-phenyl)-6-azaspiro[3.4]octan-6-yl]-[6-(5-cyclopropyl-4H-1,2,4-triazol-3-yl)-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[3-[3-[[1-(trifluoromethyl)-cyclopropyl]methylamino]-1-bicyclo[1.1.1]pentanyl]azetidin-1-yl]methanone;
3-[[2-[6-[3-(3,3-difluorocyclobutyl)-1H-1,2,4-triazol-5-yl]-2-azaspiro[3.3]heptane-2-carbonyl]-2-azaspiro[3.3]heptan-6-yl]methyl]-5-(trifluoromethyl)benzonitrile;
4-[[2-[6-(3-cyclopropyl-1H-1,2,4-triazol-5-yl)-2-azaspiro[3.3]heptane-2-carbonyl]-2,6-diazaspiro[3.3]heptan-6-yl]methyl]-2-methyl-pyrazole-3-sulfonamide;
[6-(3-cyclopropyl-1H-1,2,4-triazol-5-yl)-2-azaspiro[3.3]heptan-2-yl]-[3-[3-[5-(2,2-dimethylpropyl)-1,3,4-thiadiazol-2-yl]-1-bicyclo[1.1.1]-pentanyl]azetidin-1-yl]methanone;
[6-[3-(3,3-difluorocyclobutyl)-1H-1,2,4-triazol-5-yl]-2-azaspiro[3.3]heptan-2-yl]-[6-(4-mesyl benzyl)-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[3-[3-(5-neopentyl-1,3,4-thiadiazol-2-yl)-1-bicyclo[1.1.1]pentanyl]azetidin-1-yl]methanone;
[6-(3-cyclopropyl-1H-1,2,4-triazol-5-yl)-2-azaspiro[3.3]heptan-2-yl]-[6-[2-fluoro-4-(trifluoromethyl)benzyl]-2,6-diazaspiro[3.3]heptan-2-yl]methanone;
5-[[2-[6-[3-(1-fluorocyclopropyl)-1H-1,2,4-triazol-5-yl]-2-azaspiro[3.3]heptane-2-carbonyl]-2-azaspiro[3.3]heptan-6-yl]methyl]-2-(trifluoromethyl)benzonitrile;
[6-(2,4-difluorobenzyl)-2-azaspiro[3.3]heptan-2-yl]-[6-[3-(3,3-difluorocyclobutyl)-1H-1,2,4-triazol-5-yl]-2-azaspiro[3.3]heptan-2-yl]methanone;
[3-[3-(5-cyclopropyl-3-methyl-pyrazol-1-yl)-1-bicyclo[1.1.1]pentanyl]azetidin-1-yl]-[6-(3-cyclopropyl-1H-1,2,4-triazol-5-yl)-2-azaspiro[3.3]heptan-2-yl]methanone;
[3-[3-(5-cyclopropyl-3-methyl-pyrazol-1-yl)-1-bicyclo[1.1.1]pentanyl]azetidin-1-yl]-[6-[3-(1-hydroxycyclopropyl)-1H-1,2,4-triazol-5-yl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[[3-(trifluoromethyl)-1H-pyrazol-5-yl]methyl]-2-azaspiro[3.3]heptan-2-yl]methanone;
4-[[2-[6-(3-cyclopropyl-1H-1,2,4-triazol-5-yl)-2-azaspiro[3.3]heptane-2-carbonyl]-2-azaspiro[3.3]heptan-6-yl]methyl]-2-(trifluoromethyl)benzonitrile;
[6-[(4-tert-butylisoxazol-3-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[1-methyl-5-(trifluoromethyl)pyrazol-4-yl]oxy-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[3-[3-[[5-(trifluoromethyl)pyrazin-2-yl]amino]-1-bicyclo[1.1.1]pentanyl]azetidin-1-yl]methanone;
[6-(3-cyclopropyl-1H-1,2,4-triazol-5-yl)-2-azaspiro[3.3]heptan-2-yl]-[(6R)-6-(3-triflylbenzyl)-2-azaspiro[3.4]octan-2-yl]methanone;
[6-(3-cyclopropyl-1H-1,2,4-triazol-5-yl)-2-azaspiro[3.3]heptan-2-yl]-[3-[3-(3-methyl-1H-pyrazol-5-yl)-1-bicyclo[1.1.1]pentanyl]azetidin-1-yl]methanone;
[6-[3-(3,3-difluorocyclobutyl)-1H-1,2,4-triazol-5-yl]-2-azaspiro[3.3]heptan-2-yl]-[6-[[5-(trifluoromethyl)thiazol-2-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[2-(2,2-difluoroethyl)pyrazol-3-yl]methyl]-2,6-diazaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[5-(trifluoromethyl)thiazol-2-yl]methyl]-2,6-diazaspiro[3.3]heptan-2-yl]methanone;
[6-[3-(1-hydroxycyclopropyl)-1H-1,2,4-triazol-5-yl]-2-azaspiro[3.3]heptan-2-yl]-[(6S)-6-(4-triflylbenzyl)-2-azaspiro[3.4]octan-2-yl]methanone;
[6-(5-cyclopropyl-4H-1,2,4-triazol-3-yl)-2-azaspiro[3.3]heptan-2-yl]-[3-[4-[5-(methylamino)-1,3,4-thiadiazol-2-yl]phenyl]azetidin-1-yl]methanone;
[6-(3-cyclopropyl-1H-1,2,4-triazol-5-yl)-2-azaspiro[3.3]heptan-2-yl]-[6-[[1-(trifluoromethyl)pyrazol-3-yl]methyl]-2,6-diazaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[1-methyl-5-(trifluoromethyl)pyrazol-3-yl]methyl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[4-(trifluoromethylsulfonimidoyl)benzyl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-(3-triflylbenzyl)-2-azaspiro[3.3]heptan-2-yl]methanone;
4-[[2-[6-(3-cyclopropyl-1H-1,2,4-triazol-5-yl)-2-azaspiro[3.3]heptane-2-carbonyl]-2-azaspiro[3.3]heptan-6-yl]oxy]-2-(trifluoromethyl)benzonitrile;
5-[[2-[6-[5-(1-hydroxycyclopropyl)-4H-1,2,4-triazol-3-yl]-2-azaspiro[3.3]heptane-2-carbonyl]-2-azaspiro[3.3]heptan-6-yl]methyl]-2-(trifluoromethyl)nicotinonitrile;
[6-(3-cyclopropyl-1H-1,2,4-triazol-5-yl)-2-azaspiro[3.3]heptan-2-yl]-[6-[[5-(trifluoromethyl)-1H-pyrazol-4-yl]methyl]-2-azaspiro[3.3]heptan-2-yl]methanone;
[6-[(4-cyclobutylthiadiazol-5-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethyl)isoxazol-3-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[5-(difluoromethyl)-1H-pyrazol-3-yl]methyl]-2-azaspiro[3.3]heptan-2-yl]methanone;
5-[[(6S)-2-[6-(5-cyclopropyl-4H-1,2,4-triazol-3-yl)-2-azaspiro[3.3]heptane-2-carbonyl]-2-azaspiro[3.4]octan-6-yl]methyl]-2-(trifluoromethyl)isonicotinonitrile;
[6-(3-cyclopropyl-1H-1,2,4-triazol-5-yl)-2-azaspiro[3.3]heptan-2-yl]-[(6S)-6-(4-triflylbenzyl)-2-azaspiro[3.4]octan-2-yl]methanone;
[6-[(5-chloro-3-fluoro-2-pyridyl)methyl]-2-azaspiro[3.3]heptan-2-yl]-[6-(3-cyclopropyl-1H-1,2,4-triazol-5-yl)-2-azaspiro[3.3]heptan-2-yl]methanone:
[6-[(5-cyclobutyl-1H-pyrazol-3-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-[[5-(difluoromethyl)-1H-pyrazol-3-yl]methyl]-2-azaspiro[3.3]heptan-2-yl]-[6-[3-(1-hydroxycyclopropyl)-1H-1,2,4-triazol-5-yl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[(5-triflyl-2-pyridyl)methyl]-2-azaspiro[3.3]heptan-2-yl]methanone;
[6-[(3-chloro-5-fluoro-2-pyridyl)methyl]-2-azaspiro[3.3]heptan-2-yl]-[6-[3-(3,3-difluorocyclobutyl)-1H-1,2,4-triazol-5-yl]-2-azaspiro[3.3]heptan-2-yl]methanone;
[6-[3-cyclopropyl-1H-1,2,4-triazol-5-yl)-2-azaspiro[3.3]heptan-2-yl]-[6-[[1-(2,2,2-trifluoroethyl)pyrazol-3-yl]methyl]-2,6-diazaspiro[3.3]heptan-2-yl]methanone;
[6-[(5-chloro-2-pyridyl)oxy]-2-azaspiro[3.3]heptan-2-yl]-[6-(3-cyclopropyl-1H-1,2,4-triazol-5-yl)-2-azaspiro[3.3]heptan-2-yl]methanone;
[6-[[3-fluoro-5-(trifluoromethyl)-2-pyridyl]methyl]-2-azaspiro[3.3]heptan-2-yl]-[6-(1H-1,2,4-triazol-5-yl)-2-azaspiro[3.3]heptan-2-yl]methanone;
1-[[2-[6-[3-(1-hydroxycyclopropyl)-1H-1,2,4-triazol-5-yl]-2-azaspiro[3.3]heptane-2-carbonyl]-2-azaspiro[3.3]heptan-6-yl]methyl]-5-(trifluoromethyl)-2-pyridone;
[6-[(3-chloro-5-fluoro-2-pyridyl)oxy]-2-azaspiro[3.3]heptan-2-yl]-[6-(3-cyclopropyl-1H-1,2,4-triazol-5-yl)-2-azaspiro[3.3]heptan-2-yl]methanone;
[6-[(5-cyclopropyl-2-methyl-pyrazol-3-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[[5-(difluoromethyl)thiazol-2-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethyl)-1H-pyrazol-3-yl]methyl]-2,6-diazaspiro[3.3]heptan-2-yl]methanone;
[6-[3-(1-hydroxycyclopropyl)-1H-1,2,4-triazol-5-yl]-2-azaspiro[3.3]heptan-2-yl]-[6-([1,2,4]triazolo[1,5-a]pyridin-6-ylmethyl)-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-(3-triflylbenzyl)-2-azaspiro[3.3]heptan-2-yl]methanone;
[6-(3-cyclopropyl-1H-1,2,4-triazol-5-yl)-2-azaspiro[3.3]heptan-2-yl]-[6-[[1-(difluoromethyl)pyrazol-4-yl]methyl]-2,6-diazaspiro[3.3]heptan-2-yl]methanone;
4-[[2-[6-[3-(1-hydroxycyclopropyl)-1H-1,2,4-triazol-5-yl]-2-azaspiro[3.3]heptane-2-carbonyl]-2-azaspiro[3.3]heptan-6-yl]methyl]-2-(trifluoromethyl)benzonitrile;
[6-(5-cyclopropyl-4H-1,2,4-triazol-3-yl)-2-azaspiro[3.3]heptan-2-yl]-[6-[2-(trifluoromethyl)pyrimidin-5-yl]-2-azaspiro[3.3]heptan-2-yl]methanone;
[6-(3-cyclopropyl-1H-1,2,4-triazol-5-yl)-2-azaspiro[3.3]heptan-2-yl]-[6-[1-(2,2,2-trifluoroethyl)pyrazol-4-yl]oxy-2-azaspiro[3.3]heptan-2-yl]methanone;
[6-(3-cyclopropyl-1H-1,2,4-triazol-5-yl)-2-azaspiro[3.3]heptan-2-yl]-[3-[2-fluoro-4-(trifluoromethdylsulfonimidoyl)benzyl]oxyazetidin-1-yl]methanone;
[6-[2-mesyl-4-(trifluoromethyl)benzyl]-2-azaspiro[3.3]heptan-2-yl]-[6-(1H-1,2,4-triazol-5-yl)-2-azaspiro[3.3]heptan-2-yl]methanone;
[6-[3-(oxetan-3-yl)-1H-1,2,4-triazol-5-yl]-2-azaspiro[3.3]heptan-2-yl]-[6-[[4-(trifluoromethyl)pyrazol-1-yl]methyl]-2-azaspiro[3.3]heptan-2-yl]methanone;
[6-(1H-1,2,4-triazol-5-yl)-2-azaspiro[3.3]heptan-2-yl]-[3-[4-[1-(trifluoromethyl)cyclopropyl]phenyl]azetidin-1-yl]methanone;
[6-[3-(1-aminocyclopropyl)-1H-1,2,4-triazol-5-yl]-2-azaspiro[3.3]heptan-2-yl]-[6-[4-fluoro-2-(trifluoromethyl)benzyl]-2-azaspiro[3.3]heptan-2-yl]methanone;
[6-[(3-cyclopropyl-1,2,4-thiadiazol-5-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[3-[4-(trifluoromethylsulfonimidoyl)phenyl]azetidin-1-yl]methanone;
[6-[3-(1-hydroxycyclopropyl)-1H-1,2,4-triazol-5-yl]-2-azaspiro[3.3]heptan-2-yl]-[6-[3-(trifluoromethylsulfonimidoyl)benzyl]-2-azaspiro[3.3]heptan-2-yl]methanone;
[-(3-cyclopropyl-1H-1,2,4-triazol-5-yl)-2-azaspiro[3.3]heptan-2-yl]-[6-[[5-methyl-1-(2,2,2-trifluoroethyl)pyrazol-4-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethyl)-1H-imidazo-2-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[2-(trifluoromethyl)oxazol-5-yl]methyl]-2,6-diazaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[4-(trifluoromethyl)thiazol-2-yl]methyl]-2,6-diazaspiro[3.3]heptan-2-yl]methanone;
[6-[(3-cyclopropyl-1H-pyrazol-5-yl)methyl]-2-azaspiro[3.3]heptan-2-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-([1,2,4]triazolo[1,5-a]pyridin-6-ylmethyl)-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[3-[6-[3-hydroxy-3-(trifluoromethyl)azetidin-1-yl]-3-pyridyl]azetidin-1-yl]methanone;
[6-[3-(1-fluorocyclopropyl)-1H-1,2,4-triazol-5-yl]-2-azaspiro[3.3]heptan-2-yl]-[6-[[5-(trifluoromethyl)pyrimidin-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-(3-hydroxy-1,2,4-triazol-1-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[3-fluoro-5-(trifluoromethylsulfonimidoyl)benzyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[2-methyl-5-(trifluoromethyl)pyrazol-3-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[2-(difluoromethyl)thiazol-5-yl]methyl]-2,6-diazaspiro[3.3]heptan-2-yl]methanone;
[6-[3-(1-fluorocyclopropyl)-1H-1,2,4-triazol-5-yl]-2-azaspiro[3.3]heptan-2-yl]-[6-[(5-triflyl-2-pyridyl)methyl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[(6R)-6-(4-triflylbenzyl)-2-azaspiro[3.4]octan-2-yl]methanone;
[6-(3-cyclopropyl-1H-1,2,4-triazol-5-yl)-2-azaspiro[3.3]heptan-2-yl]-[6-(3-fluoro-5-triflyl-benzyl)-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[4-(trifluoromethyl)isothiazol-5-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[2-(trifluoromethyl)pyrazol-3-yl]methyl]-2,6-diazaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[3-(trifluoromethylsulfonimidoyl)phenyl]methyl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[2-methyl-4-(trifluoromethyl)pyrazol-3-yl]methyl]-2,6-diazaspiro[3.3]heptan-2-yl]methanone;
[6-[(4-tert-butyl-1H-imidazol-2-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[3-(methylsulfonimidoyl)-5-(trifluoromethyl)benzyl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[5-(trifluoromethyl)-2-pyridyl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)-1,2,4-oxadiazol-5-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)isoxazol-4-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[2-methyl-4-(trifluoromethyl)pyrazol-3-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[3-fluoro-5-(trifluoromethyl)-2-pyridyl]oxy]-2-azaspiro[3.3]heptan-2-yl]methanone;
[6-(3-cyclopropyl-1H-1,2,4-triazol-5-yl)-2-azaspiro[3.3]heptan-2-yl]-[3-[2-[(3R)-3-(trifluoromethyl)pyrrolidino]pyrimidin-5-yl]azetidin-1-yl]methanone;
[6-(3-cyclopropyl-1H-1,2,4-triazol-5-yl)-2-azaspiro[3.3]heptan-2-yl]-[(6R)-6-(4-triflylbenzyl)-2-azaspiro[3.4]octan-2-yl]methanone;
[6-[3-(3,3-difluorocyclobutyl)-1H-1,2,4-triazol-5-yl]-2-azaspiro[3.3]heptan-2-yl]-[6-[[3-(trifluoromethylsulfonimidoyl)phenyl]methyl]-2-azaspiro[3.3]heptan-2-yl]methanone; 5-[[(6R)-2-[6-(5-cyclopropyl-4H-1,2,4-triazol-3-yl)-2-azaspiro[3.3]heptane-2-carbonyl]-2-azaspiro[3.4]octan-6-yl]methyl]-2-(trifluoromethyl)isonicotinonitrile;
[6-[3-(1-hydroxycyclopropyl)-1H-1,2,4-triazol-5-yl]-2-azaspiro[3.3]heptan-2-yl]-[6-[3-mesyl-5-(trifluoromethyl)benzyl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[[4-(trifluoromethyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)isoxazol-5-yl]methyl]-2,6-diazaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[[3-(trifluoromethyl)pyrazol-1-yl]methyl]-2-azaspiro[3.3]heptan-2-yl]methanone;
[3-[3-(4-fluorophenyl)-1-bicyclo[1.1.1]pentanyl]azetidin-1-yl]-[6-[3-(1-hydroxycyclopropyl)-1H-1,2,4-triazol-5-yl]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethyl)isothiazol-3-yl]methyl]-2,6-diazaspiro[3.3]heptan-2-yl]methanone;
[6-[(5-chloro-3-fluoro-2-pyridyl)methyl]-2-azaspiro[3.3]heptan-2-yl]-[6-[3-(1-hydroxycyclopropyl)-1H-1,2,4-triazol-5-yl]-2-azaspiro[3.3]heptan-2-yl]methanone;
4-[[2-[6-[3-(3,3-difluorocyclobutyl)-1H-1,2,4-triazol-5-yl]-2-azaspiro[3.3]heptane-2-carbonyl]-2-azaspiro[3.3]heptan-6-yl]methyl]-2-(trifluoromethyl)benzonitrile;
[6-(5-cyclopropyl-4H-1,2,4-triazol-3-yl)-2-azaspiro[3.3]heptan-2-yl]-[6-[3-fluoro-5-(trifluoromethyl)-2-pyridyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[2-(2,2,2-trifluoroethyl)-5-(trifluoromethyl)pyrazol-3-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-(3-dimethylphosphoryl-5-fluoro-benzyl)-2-azaspiro[3.3]heptan-2-yl]methanone;
[6-(3-cyclopropyl-1H-1,2,4-triazol-5-yl)-2-azaspiro[3.3]heptan-2-yl]-[6-[[1-methyl-5-(trifluoromethyl)pyrazol-3-yl]methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[5-(trifluoromethyl)-2-pyridyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)-1,2,4-oxadiazol-5-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-(1H-pyrazolo[4,3-b]pyridin-5-ylmethyl)-2-azaspiro[3.3]heptan-2-yl]methanone;
[6-(3-cyclopropyl-1H-1,2,4-triazol-5-yl)-2-azaspiro[3.3]heptan-2-yl]-[3-[3-(4-fluoro-2-mesyl-phenyl)-1-bicyclo[1.1.1]pentanyl]azetidin-1-yl]methanone;
[6-[(3-cyclopropyl-5-methyl-isoxazol-4-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[4-(trifluoromethylsulfonimidoyl)phenyl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-methyl-5-(trifluoromethyl)pyrazol-4-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[2-(2,2,2-trifluoroethyl)triazol-4-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[4-(2,2,2-trifluoroethyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[2-methyl-4-(trifluoromethyl)pyrazol-3-yl]methyl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[6-(1H-pyrazolo[4,3-b]pyridin-5-ylmethyl)-2-azaspiro[3.3]heptan-2-yl]methanone;
[6-(3-cyclopropyl-1H-1,2,4-triazol-5-yl)-2-azaspiro[3.3]heptan-2-yl]-[6-[[4-(trifluoromethyl)oxazol-2-yl]methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[5-(trifluoromethyl)pyrimidin-2-yl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[3-(2,2,2-trifluoroethyl)-1,2,4-oxadiazol-5-yl]methyl]-2,6-diazaspiro[3.3]heptan-2-yl]methanone;
5-[[(6S)-2-[6-[3-(1-hydroxy-cyclopropyl)-1H-1,2,4-triazol-5-yl]-2-azaspiro[3.3]heptane-2-carbonyl]-2-azaspiro[3.4]octan-6-yl]oxy]-2-(trifluoromethyl)isonicotinonitrile;
[6-[3-(1-hydroxycyclopropyl)-1H-1,2,4-triazol-5-yl]-2-azaspiro[3.3]heptan-2-yl]-[3-[4-(trifluoromethylsulfonimidoyl)phenyl]azetidin-1-yl]methanone;
[6-(3-cyclopropyl-1H-1,2,4-triazol-5-yl)-2-azaspiro[3.3]heptan-2-yl]-[6-[[1-(2,2,2-trifluoroethyl)imidazol-2-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[3-[3-[[5-(trifluoromethyl)pyrazin-2-yl]-amino]-1-bicyclo[1.1.1]-pentanyl]azetidin-1-yl]methanone;
[6-(3-cyclopropyl-1H-1,2,4-triazol-5-yl)-2-azaspiro[3.3]heptan-2-yl]-[6-[[4-(trifluoromethyl)isothiazol-5-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethyl)oxazol-4-yl]methyl]-2,6-diazaspiro[3.3]heptan-2-yl]methanone;
[3-[3-(3-cyclopropyl-1H-pyrazol-5-yl)-1-bicyclo[1.1.1]pentanyl]azetidin-1-yl]-[6-[3-(3,3-difluorocyclobutyl)-1H-1,2,4-triazol-5-yl]-2-azaspiro[3.3]heptan-2-yl]methanone;
[6-(3-cyclopropyl-1H-1,2,4-triazol-5-yl)-2-azaspiro[3.3]heptan-2-yl]-[7-(4-triflylbenzyl)-2,7-di azaspiro[3.4]octan-2-yl]methanone;
[6-(3-cyclopropyl-1H-1,2,4-triazol-5-yl)-2-azaspiro[3.3]heptan-2-yl]-[6-[[4-(trifluoromethyl)thiazol-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[3-[3-(3-fluoro-5-methoxy-phenyl)-1-bicyclo[1.1.1]pentanyl]azetidin-1-yl]methanone;
[6-[3-(1-hydroxycyclopropyl)-1H-1,2,4-triazol-5-yl]-2-azaspiro-[3.3]heptan-2-yl]-[4-[3-[5-[1-(trifluoromethyl)cyclopropyl]-1,3,4-oxadiazol-2-yl]-1-bicyclo-[1.1.1]pentanyl]piperidino]methanone;
[6-(3-cyclopropyl-1H-1,2,4-triazol-5-yl)-2-azaspiro[3.3]heptan-2-yl]-[3-[3-[5-(trifluoromethyl)pyrazin-2-yl]oxy-1-bicyclo[1.1.1]pentanyl]azetidin-1-yl]methanone;
[6-(3-cyclopropyl-1H-1,2,4-triazol-5-yl)-2-azaspiro[3.3]heptan-2-yl]-[3-[2-[(3S)-3-(trifluoromethyl)pyrrolidino]pyrimidin-5-yl]azetidin-1-yl]methanone;
[6-(3-cyclopropyl-1H-1,2,4-triazol-5-yl)-2-azaspiro[3.3]heptan-2-yl]-[6-[[1-(trifluoromethyl)imidazol-2-yl]methyl]-2,6-diazaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[[6-(trifluoromethoxy)-3-pyridyl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[4-[1-(trifluoromethyl)cyclopropyl]-pyrazol-1-yl]methyl]-2-azaspiro[3.3]heptan-2-yl]methanone;
[3-[3-(3,5-dimethylpyrazol-1-yl)-1-bicyclo[1.1.1]pentanyl]azetidin-1-yl]-[6-[3-(1-hydroxycyclopropyl)-1H-1,2,4-triazol-5-yl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)isothiazol-5-yl]methyl]-2,6-diazaspiro[3.3]heptan-2-yl]methanone;
[6-[3-(1-hydroxycyclopropyl)-1H-1,2,4-triazol-5-yl]-2-azaspiro[3.3]heptan-2-yl]-[7-(4-triflylbenzyl)-2,7-diazaspiro[3.4]octan-2-yl]methanone;
[6-[2-fluoro-4-(trifluoromethylsulfonimidoyl)benzyl]-2-azaspiro[3.3]heptan-2-yl]-[6-[3-(1-hydroxycyclopropyl)-1H-1,2,4-triazol-5-yl]-2-azaspiro[3.3]heptan-2-yl]methanone;
[6-[3-(oxetan-3-yl)-1H-1,2,4-triazol-5-yl]-2-azaspiro[3.3]heptan-2-yl]-[6-[[5-(trifluoromethyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[2-(2,2,2-trifluoroethyl)pyrazol-3-yl]methyl]-2,6-diazaspiro[3.3]heptan-2-yl]methanone;
3-[[2-[6-(3-cyclopropyl-1H-1,2,4-triazol-5-yl)-2-azaspiro[3.3]heptane-2-carbonyl]-2,6-diazaspiro[3.3]heptan-6-yl]methyl]-2-pyridone;
1-[[2-[6-[3-(1-hydroxycyclopropyl)-1H-1,2,4-triazol-5-yl]-2-azaspiro[3.3]heptane-2-carbonyl]-2-azaspiro[3.3]heptan-6-yl]methyl]pyrazole-4-carbonitrile;
[6-(3-cyclopropyl-1H-1,2,4-triazol-5-yl)-2-azaspiro[3.3]heptan-2-yl]-[6-[[2-methyl-5-(trifluoromethyl)triazol-4-yl]methyl]-2-azaspiro[3.3]heptan-2-yl]methanone;
[6-(1H-1,2,4-triazol-5-yl)-2-azaspiro[3.3]heptan-2-yl]-[6-[[5-(trifluoromethoxy)-2-pyridyl]methyl]-2-azaspiro[3.3]heptan-2-yl]methanone;
[6-(3-cyclopropyl-1H-1,2,4-triazol-5-yl)-2-azaspiro[3.3]heptan-2-yl]-[3-[3-[4-(trifluoromethyl)phenyl]sulfonyl-1-bicyclo[1.1.1]pentanyl]azetidin-1-yl]methanone;
[6-(3-cyclopropyl-1H-1,2,4-triazol-5-yl)-2-azaspiro[3.3]heptan-2-yl]-[7-[4-(trifluoromethyl)phenyl]sulfonyl-2-azaspiro[3.5]nonan-2-yl]methanone;
1-[[2-[6-(3-cyclopropyl-1H-1,2,4-triazol-5-yl)-2-azaspiro[3.3]heptane-2-carbonyl]-2-azaspiro[3.3]heptan-6-yl]methyl]-5-(difluoromethoxy)-2-pyridone;
[6-(3-cyclopropyl-1H-1,2,4-triazol-5-yl)-2-azaspiro[3.3]heptan-2-yl]-[3-[3-(3,5-dimethylpyrazol-1-yl)-1-bicyclo[1.1.1]pentanyl]azetidin-1-yl]methanone;
[6-[3-(1-hydroxycyclopropyl)-1H-1,2,4-triazol-5-yl]-2-azaspiro[3.3]heptan-2-yl]-[(6S)-6-(3-triflylbenzyl)-2-azaspiro[3.4]octan-2-yl]methanone;
[6-[3-(1-hydroxycyclopropyl)-1H-1,2,4-triazol-5-yl]-2-azaspiro[3.3]heptan-2-yl]-[6-[[1-(trifluoromethyl)pyrazol-4-yl]methyl]-2-azaspiro[3.3]heptan-2-yl]methanone;
[6-[3-(oxetan-3-yl)-1H-1,2,4-triazol-5-yl]-2-azaspiro[3.3]heptan-2-yl]-[6-(3-triflylbenzyl)-2-azaspiro[3.3]heptan-2-yl]methanone;
[6-(3-cyclopropyl-1H-1,2,4-triazol-5-yl)-2-azaspiro[3.3]heptan-2-yl]-[6-[[1-methyl-5-(trifluoromethyl)pyrazol-4-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[4-(trifluoromethylsulfonimidoyl)phenoxy]-2-azaspiro[3.3]heptan-2-yl]methanone;
[6-(3-cyclopropyl-1H-1,2,4-triazol-5-yl)-2-azaspiro[3.3]heptan-2-yl]-[6-[(5-triflyl-3-pyridyl)methyl]-2-azaspiro[3.3]heptan-2-yl]methanone;
[6-(3-cyclopropyl-1H-1,2,4-triazol-5-yl)-2-azaspiro[3.3]heptan-2-yl]-[7-[[4-(trifluoromethyl)thiazol-2-yl]methyl]-2,7-diazaspiro[3.5]nonan-2-yl]methanone;
[6-[[3-(cyclopropylmethyl)-1,2,4-oxadiazol-5-yl]methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-(1H-1,2,4-triazol-5-yl)-2-azaspiro[3.3]heptan-2-yl]-[6-[3-(trifluoromethylsulfonimidoyl)benzyl]-2-azaspiro[3.3]heptan-2-yl]methanone;
[6-(3-cyclopropyl-1H-1,2,4-triazol-5-yl)-2-azaspiro[3.3]heptan-2-yl]-[6-[4-fluoro-2-(methylsulfonimidoyl)benzyl]-2-azaspiro[3.3]heptan-2-yl]methanone;
[6-(3-cyclopropyl-1H-1,2,4-triazol-5-yl)-2-azaspiro[3.3]heptan-2-yl]-[3-[3-[[5-(trifluoromethyl)-2-pyridyl]oxy]-1-bicyclo[1.1.1]pentanyl]azetidin-1-yl]methanone;
[6-[(5-chloro-3-fluoro-2-pyridyl)methyl]-2-azaspiro[3.3]heptan-2-yl]-[6-[3-(3,3-difluorocyclobutyl)-1H-1,2,4-triazol-5-yl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[5-[1-(trifluoromethyl)cyclopropyl]-1,3,4-oxadiazol-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethylsulfonimidoyl)-2-pyridyl]methyl]-2-azaspiro[3.3]heptan-2-yl]methanone;
[6-[(4-tert-butylthiazol-2-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[(6R)-6-(3-triflylbenzyl)-2-azaspiro[3.4]octan-2-yl]methanone;
[6-[3-fluoro-5-(trifluoromethyl)benzyl]-2-azaspiro[3.3]heptan-2-yl]-[6-[3-(oxetan-3-yl)-1H-1,2,4-triazol-5-yl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(1,1-difluoroethyl)-1,2,4-oxadiazol-5-yl]methyl]-2,6-diazaspiro[3.3]heptan-2-yl]methanone;
[6-[[1-methyl-5-(trifluoromethyl)pyrazol-3-yl]methyl]-2-azaspiro[3.3]heptan-2-yl]-[6-[3-(oxetan-3-yl)-1H-1,2,4-triazol-5-yl]-2-azaspiro[3.3]heptan-2-yl]methanone;
[3-[4-(cyclopropyisulfonimidoyl)phenyl]azetidin-1-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-(3-cyclopropyl-1,2,4-triazol-1-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[[4-(trifluoromethyl)-1H-pyrazol-3-yl]methyl]-2-azaspiro[3.3]heptan-2-yl]methanone;
[6-[3-(oxetan-3-yl)-1H-1,2,4-triazol-5-yl]-2-azaspiro[3.3]heptan-2-yl]-[6-[[5-(trifluoromethyl)-3-pyridyl]methyl]-2-azaspiro[3.3]heptan-2-yl]methanone;
3-[[2-[6-[3-(1-hydroxycyclopropyl)-1H-1,2,4-triazol-5-yl]-2-azaspiro[3.3]heptane-2-carbonyl]-2-azaspiro[3.3]heptan-6-yl]methyl]-5-(trifluoromethyl)benzonitrile;
[6-[3-(3,3-difluorocyclobutyl)-1H-1,2,4-triazol-5-yl]-2-azaspiro-[3.3]heptan-2-yl]-[6-[[5-(trifluoromethyl)pyrazin-2-yl]methyl]-2,6-diazaspiro[3.3]heptan-2-yl]methanone;
[3-[4-(3,5-diisopropylpyrazol-1-yl)phenyl]azetidin-1-yl]-[6-[3-(1-hydroxycyclopropyl)-1H-1,2,4-triazol-5-yl]-2-azaspiro[3.3]heptan-2-yl]methanone;
3-fluoro-4-[[2-[6-[3-(1-hydroxycyclopropyl)-1H-1,2,4-triazol-5-yl]-2-azaspiro[3.3]heptane-2-carbonyl]-2-azaspiro[3.3]heptan-6-yl]methyl]benzonitrile;
[6-[3-mesyl-4-(trifluoromethyl)benzyl]-2-azaspiro[3.3]heptan-2-yl]-[6-(1H-1,2,4-triazol-5-yl)-2-azaspiro[3.3]heptan-2-yl]methanone;
[6-[4-fluoro-2-(trifluoromethyl)benzyl]-2-azaspiro[3.3]heptan-2-yl]-[6-[3-(oxetan-3-yl)-1H-1,2,4-triazol-5-yl]-2-azaspiro[3.3]heptan-2-yl]methanone;
[6-[3-(oxetan-3-yl)-1H-1,2,4-triazol-5-yl]-2-azaspiro[3.3]heptan-2-yl]-[6-(4-triflylbenzyl)-2-azaspiro[3.3]heptan-2-yl]methanone;
[7-[(5-chloro-2-pyridyl)methyl]-2-azaspiro[3.5]nonan-2-yl]-[6-[3-(3,3-difluorocyclobutyl)-1H-1,2,4-triazol-5-yl]-2-azaspiro[3.3]heptan-2-yl]methanone;
1-[[2-[6-(3-cyclopropyl-1H-1,2,4-triazol-5-yl)-2-azaspiro[3.3]heptane-2-carbonyl]-2-azaspiro[3.3]heptan-6-yl]methyl]-5-(trifluoromethoxy)-2-pyridone;
[6-[3-(3,3-difluorocyclobutyl)-1H-1,2,4-triazol-5-yl]-2-azaspiro[3.3]heptan-2-yl]-[6-[[3-(trifluoromethyl)isoxazol-5-yl]methyl]-2,6-diazaspiro[3.3]heptan-2-yl]methanone;
3-[[2-[6-[3-(oxetan-3-yl)-1H-1,2,4-triazol-5-yl]-2-azaspiro[3.3]heptane-2-carbonyl]-2-azaspiro[3.3]heptan-6-yl]methyl]-5-(trifluoromethyl)benzonitrile;
[6-(3-cyclopropyl-1H-1,2,4-triazol-5-yl)-2-azaspiro[3.3]heptan-2-yl]-[3-[3-(5-fluoro-3-pyridyl)-1-bicyclo[1.1.1]pentanyl]azetidin-1-yl]methanone;
[6-[3-(oxetan-3-yl)-1H-1,2,4-triazol-5-yl]-2-azaspiro[3.3]heptan-2-yl]-[6-[[5-(trifluoromethyl)pyrazin-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[(3-ethyl-1,2,4-thiadiazol-5-yl)methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[2-(trifluoromethyl)oxazol-4-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[1-(2,2,2-trifluoroethyl)pyrazol-4-yl]methyl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[[1-(2,2,2-trifluoroethyl)pyrazol-4-yl]methyl]-2-azaspiro[3.3]heptan-2-yl]methanone;
1-[[2-[6-(3-cyclopropyl-1H-1,2,4-triazol-5-yl)-2-azaspiro[3.3]heptane-2-carbonyl]-2-azaspiro[3.3]heptan-6-yl]methyl]-4-(trifluoromethyl)pyridin-2-one;
5-chloro-2-[3-[1-[6-(5-cyclopropyl-4H-1,2,4-triazol-3-yl)-2-azaspiro[3.3]heptane-2-carbonyl]azetidin-3-yl]-1-bicyclo[1.1.1]pentanyl]-N-ethyl-benzamide;
[6-[2-fluoro-4-(trifluoromethyl)benzyl]-2-azaspiro[3.3]heptan-2-yl]-[6-[3-(oxetan-3-yl)-1H-1,2,4-triazol-5-yl]-2-azaspiro[3.3]heptan-2-yl]methanone;
[6-[3-(oxetan-3-yl)-1H-1,2,4-triazol-5-yl]-2-azaspiro[3.3]heptan-2-yl]-[6-[[6-(trifluoromethoxy)-3-pyridyl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-methyl-3-(trifluoromethyl)pyrazol-4-yl]methyl]-2-azaspiro[3.3]heptan-2-yl]methanone;
4-[[2-[6-[3-(oxetan-3-yl)-1H-1,2,4-triazol-5-yl]-2-azaspiro[3.3]heptane-2-carbonyl]-2-azaspiro[3.3]heptan-6-yl]methyl]-2-(trifluoromethyl)benzonitrile;
4-[[2-[6-(3-cyclopropyl-1H-1,2,4-triazol-5-yl)-2-azaspiro[3.3]heptane-2-carbonyl]-2-azaspiro[3.3]heptan-6-yl]methyl]-3-fluoro-benzonitrile;
[6-(3-cyclopropyl-1H-1,2,4-triazol-5-yl)-2-azaspiro[3.3]heptan-2-yl]-[3-[3-(4-fluorophenyl)-1-bicyclo[1.1.1]pentanyl]azetidin-1-yl]methanone;
[6-[[1-methyl-5-(trifluoromethyl)pyrazol-4-yl]methyl]-2-azaspiro[3.3]heptan-2-yl]-[6-[3-(oxetan-3-yl)-1H-1,2,4-triazol-5-yl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[[2-methyl-5-(trifluoromethyl)pyrazol-3-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[3-[4-(1-mesylcyclopropyl)phenyl]azetidin-1-yl]methanone;
[6-[3-(1-hydroxycyclopropyl)-1H-1,2,4-triazol-5-yl]-2-azaspiro[3.3]heptan-2-yl]-[6-[[2-methyl-4-(trifluoromethyl)pyrazol-3-yl]methyl]-2-azaspiro[3.3]heptan-2-yl]methanone;
4-[[2-[6-(5-cyclopropyl-4H-1,2,4-triazol-3-yl)-2-azaspiro[3.3]heptane-2-carbonyl]-2-azaspiro[3.3]heptan-6-yl]methyl]-6-(trifluoromethyl)picolinonitrile;
[6-(3-cyclopropyl-1H-1,2,4-triazol-5-yl)-2-azaspiro[3.3]heptan-2-yl]-[7-[[5-(trifluoromethyl)-1,3,4-oxadiazol-2-yl]methyl]-2-azaspiro[3.5]nonan-2-yl]methanone;
[6-(3-cyclopropyl-1H-1,2,4-triazol-5-yl)-2-azaspiro[3.3]heptan-2-yl]-[6-[[4-(trifluoromethyl)thiazol-2-yl]methyl]-2,6-diazaspiro[3.3]heptan-2-yl]methanone;
[6-[3-(1-hydroxycyclopropyl)-1H-1,2,4-triazol-5-yl]-2-azaspiro[3.3]heptan-2-yl]-[3-[4-(2-mesylphenyl)phenyl]azetidin-1-yl]methanone;
[6-[3-(oxetan-3-yl)-1H-1,2,4-triazol-5-yl]-2-azaspiro[3.3]heptan-2-yl]-[6-[[4-(trifluoromethylsulfonimidoyl)phenyl]methyl]-2-azaspiro[3.3]heptan-2-yl]methanone;
[6-[3-(oxetan-3-yl)-1H-1,2,4-triazol-5-yl]-2-azaspiro[3.3]heptan-2-yl]-[6-[[4-(trifluoromethyl)thiazol-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[2-(trifluoromethyl)thiazol-5-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[2-methyl-5-(trifluoromethyl)pyrazol-3-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[3-[3-(4-methylsulfonylphenyl)-1-bicyclo[1.1.1]pentanyl]azetidin-1-yl]methanone;
[6-(3-cyclopropyl-1H-1,2,4-triazol-5-yl)-2-azaspiro[3.3]heptan-2-yl]-[6-[[3-[1-(trifluoromethyl)cyclopropyl]-1,2,4-triazol-1-yl]methyl]-2-azaspiro[3.3]heptan-2-yl]methanone;
[6-[[5-(trifluoromethyl)pyrazin-2-yl]methyl]-2-azaspiro[3.3]heptan-2-yl]-[6-[3-(trifluoromethyl)-1H-1,2,4-triazol-5-yl]-2-azaspiro[3.3]heptan-2-yl]methanone;
[6-[(3-chloro-5-fluoro-2-pyridyl)methyl]-2-azaspiro[3.3]heptan-2-yl]-[6-(1H-1,2,4-triazol-5-yl)-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[3-[4-(1-mesylcyclopropyl)phenyl]azetidin-1-yl]methanone;
[6-(4-mesylbenzyl)-2-azaspiro[3.3]heptan-2-yl]-[6-[3-(trifluoromethyl)-1H-1,2,4-triazol-5-yl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[5-[1-(trifluoromethyl);
cyclopropyl]-1,3,4-oxadiazol-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-(2,2,2-trifluoroethyl)-5-(trifluoromethyl)pyrazol-3-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethyl)-1,2,4-oxadiazol-3-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethyl)-1H-imidazol-4-yl]methyl]-2,6-diazaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[[5-(trifluoromethyl)pyrazol-1-yl]methyl]-2-azaspiro[3.3]heptan-2-yl]methanone;
3-fluoro-4-[[2-[6-[3-(oxetan-3-yl)-1H-1,2,4-triazol-5-yl]-2-azaspiro[3.3]heptane-2-carbonyl]-2-azaspiro[3.3]heptan-6-yl]methyl]benzonitrile;
[6-(3-cyclopropyl-1H-1,2,4-triazol-5-yl)-2-azaspiro[3.3]heptan-2-yl]-[3-[3-[difluoro-[4-(trifluoromethyl)-phenyl]methyl]-1-bicyclo[1.1.1]-pentanyl]azetidin-1-yl]methanone;
[6-(3-cyclopropyl-1H-1,2,4-triazol-5-yl)-2-azaspiro[3.3]heptan-2-yl]-[6-(1H-pyrazol-3-ylmethyl)-2,6-diazaspiro[3.3]heptan-2-yl]methanone;
[3-[3-(5-neopentyl-1,3,4-thiadiazol-2-yl)-1-bicyclo[1.1.1]pentanyl]azetidin-1-yl]-[6-[3-(oxetan-3-yl)-1H-1,2,4-triazol-5-yl]-2-azaspiro[3.3]heptan-2-yl]methanone;
1-[[2-[6-(3-cyclopropyl-1H-1,2,4-triazol-5-yl)-2-azaspiro[3.3]heptane-2-carbonyl]-2-azaspiro[3.3]heptan-6-yl]methyl]pyrazole-4-carbonitrile;
[6-(5-cyclopropyl-4H-1,2,4-triazol-3-yl)-2-azaspiro[3.3]heptan-2-yl]-[3-[4-(2,5-dimethylthiazol-4-yl)phenyl]azetidin-1-yl]methanone;
[6-(5-cyclopropyl-4H-1,2,4-triazol-3-yl)-2-azaspiro[3.3]heptan-2-yl]-[6-[2-mesyl-4-(trifluoromethyl)phenyl]-2-azaspiro[3.3]heptan-2-yl]methanone;
[6-(3-cyclopropyl-1H-1,2,4-triazol-5-yl)-2-azaspiro[3.3]heptan-2-yl]-[7-[[6-(trifluoromethyl)-3-pyridyl]methyl]-2,7-diazaspiro[3.4]octan-2-yl]methanone;
[6-(3-cyclopropyl-1H-1,2,4-triazol-5-yl)-2-azaspiro[3.3]heptan-2-yl]-[6-[6-(trifluoromethyl)-3-pyridyl]-2-azaspiro[3.3]heptan-2-yl]methanone;
[6-(5-cyclopropyl-4H-1,2,4-triazol-3-yl)-2-azaspiro[3.3]heptan-2-yl]-[3-[4-(4-methylimidazol-1-yl)phenyl]azetidin-1-yl]methanone;
[6-(3-cyclopropyl-1H-1,2,4-triazol-5-yl)-2-azaspiro[3.3]heptan-2-yl]-[3-[3-[[5-(trifluoromethyl)-2-pyridyl]-sulfonyl]-1-bicyclo[1.1.1]pentanyl]-azetidin-1-yl]methanone;
5-[[2-[6-[3-(oxetan-3-yl)-1H-1,2,4-triazol-5-yl]-2-azaspiro[3.3]heptane-2-carbonyl]-2-azaspiro[3.3]heptan-6-yl]methyl]-2-(trifluoromethyl)benzonitrile;
[6-(3-cyclopropyl-1H-1,2,4-triazol-5-yl)-2-azaspiro[3.3]heptan-2-yl]-[6-[3-mesyl-5-(trifluoromethyl)benzyl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[[1-methyl-3-(trifluoromethyl)pyrazol-4-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[4-[[5-(trifluoromethyl)pyrazin-2-yl]amino]cuban-1-yl]methanone;
[6-[3-(1-hydroxycyclopropyl)-1H-1,2,4-triazol-5-yl]-2-azaspiro[3.3]heptan-2-yl]-[3-[4-(3-neopentyltriazol-4-yl)phenyl]azetidin-1-yl]methanone;
[6-(3-cyclopropyl-1H-1,2,4-triazol-5-yl)-2-azaspiro[3.3]heptan-2-yl]-[6-[(5-triflyl-2-pyridyl)methyl]-2-azaspiro[3.3]heptan-2-yl]methanone;
[6-[3-(oxetan-3-yl)-1H-1,2,4-triazol-5-yl]-2-azaspiro[3.3]heptan-2-yl]-[7-[[5-(trifluoromethyl)-2-pyridyl]methyl]-2-azaspiro[3.5]nonan-2-yl]methanone;
[6-[3-(1-hydroxycyclopropyl)-1H-1,2,4-triazol-5-yl]-2-azaspiro[3.3]heptan-2-yl]-[6-[[1-methyl-5-trifluoromethyl)pyrazol-4-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[5-[1-(trifluoromethyl)cyclopropyl]-1H-1,2,4-triazol-3-yl]methyl]-2-azaspiro[3.3]heptan-2-yl]methanone;
[6-(4-mesylbenzyl)-2-azaspiro[3.3]heptan-2-yl]-[6-[3-(oxetan-3-yl)-1H-1,2,4-triazol-5-yl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(difluoromethyl)-5-methyl-pyrazol-1-yl]methyl]-2-azaspiro[3.3]heptan-2-yl]methanone;
[6-[(3,5-difluoro-2-pyridyl)methyl]-2-azaspiro[3.3]heptan-2-yl]-[6-[3-(trifluoromethyl)-1H-1,2,4-triazol-5-yl]-2-azaspiro[3.3]heptan-2-yl]methanone;
[3-[4-(3-tert-butyl-5-methyl-pyrazol-1-yl)phenyl]azetidin-1-yl]-[6-[3-(1-hydroxycyclopropyl)-1H-1,2,4-triazol-5-yl]-2-azaspiro[3.3]heptan-2-yl]methanone;
[3-[4-(3-cyclopropyl-5-methyl-pyrazol-1-yl)phenyl]azetidin-1-yl]-[6-[3-(1-hydroxycyclopropyl)-1H-1,2,4-triazol-5-yl]-2-azaspiro[3.3]heptan-2-yl]methanone;
[6-[(3-cyclopropylpyrazol-1-yl)methyl]-2-azaspiro[3.3]heptan-2-yl]-[6-(3-cyclopropyl-1H-1,2,4-triazol-5-yl)-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[3-[4-[1-methyl-3-(trifluoromethyl)pyrazol-4-yl]phenyl]azetidin-1-yl]methanone;
[6-[3-(oxetan-3-yl)-1H-1,2,4-triazol-5-yl]-2-azaspiro[3.3]heptan-2-yl]-[6-[[5-(trifluoromethyl)-2-pyridyl]methyl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[3-[3-[[1-(trifluoromethyl)cyclopropyl]methylamino]-1-bicyclo[1.1.1]pentanyl]azetidin-1-yl]methanone;
[6-(5-cyclopropyl-4H-1,2,4-triazol-3-yl)-2-azaspiro[3.3]heptan-2-yl]-[6-[difluoro-[6-(trifluoromethyl)-3-pyridyl]methyl]-2-azaspiro[3.3]heptan-2-yl]methanone;
[6-[2-mesyl-4-(trifluoromethyl)benzyl]-2-azaspiro[3.3]heptan-2-yl]-[6-[3-(oxetan-3-yl)-1H-1,2,4-triazol-5-yl]-2-azaspiro[3.3]heptan-2-yl]methanone;
6-(5-cyclopropyl-4H-1,2,4-triazol-3-yl)-N-[[6-(trifluoromethyl)-3,4-diazatricyclo[7.1.1.03,7]undeca-4,6-lien-1-yl]methyl]-2-azaspiro[3.3]heptane-2-carboxamide;
[6-[3-(1-hydroxycyclopropyl)-1H-1,2,4-triazol-5-yl]-2-azaspiro[3.3]heptan-2-yl]-[6-[6-(trifluoromethyl)-3-pyridyl]-2-azaspiro[3.3]heptan-2-yl]methanone;
[6-[3-(oxetan-3-yl)-1H-1,2,4-triazol-5-yl]-2-azaspiro[3.3]heptan-2-yl]-[6-[4-(trifluoromethylsuifonimidoyl)benzyl]-2-azaspiro[3.3]heptan-2-yl]methanone;
[6-[(5-chloro-2-pyridyl)methyl]-2-azaspiro[3.3]heptan-2-yl]-[6-(1H-1,2,4-triazol-5-yl)-2-azaspiro[3.3]heptan-2-yl]methanone;
[3-[4-(4-cyclopropylimidazol-1-yl)phenyl]azetidin-1-yl]-[6-(5-cyclopropyl-4H-1,2,4-triazol-3-yl)-2-azaspiro[3.3]heptan-2-yl]methanone;
5-[[2-[6-(5-cyclopropyl-4H-1,2,4-triazol-3-yl)-2-azaspiro[3.3]heptane-2-carbonyl]-2-azaspiro[3.3]heptan-6-yl]methyl]-2-(trifluoromethyl)nicotinonitrile;
[6-(5-cyclopropyl-4H-1,2,4-triazol-3-yl)-2-azaspiro[3.3]heptan-2-yl]-[3-[4-[1-methyl-3-(trifluoromethyl)pyrazol-4-yl]phenyl]azetidin-1-yl]methanone;
[6-(2,4-difluorobenzyl)-2-azaspiro[3.3]heptan-2-yl]-[6-[3-(oxetan-3-yl)-1H-1,2,4-triazol-5-yl]-2-azaspiro[3.3]heptan-2-yl]methanone;
[6-(3-cyclopropyl-1H-1,2,4-triazol-5-yl)-2-azaspiro[3.3]heptan-2-yl]-[2-[[3-methyl-5-(trifluoromethyl)-2-pyridyl]oxy]-7-azaspiro[3.5]nonan-7-yl]methanone;
[6-[3-(3,3-difluorocyclobutyl)-1H-1,2,4-triazol-5-yl]-2-azaspiro[3.3]heptan-2-yl]-[6-[[4-(trifluoromethyl)triazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[(4-dimethylphosphoryl-2-methyl-pyrazol-3-yl)methyl]-2,6-diazaspiro[3.3]heptan-2-yl]methanone;
[3-[4-[5-cyclopropyl-3-(difluoromethyl)pyrazol-1-yl]phenyl]azetidin-1-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[3-[4-[4-(1-hydroxyethyl)-5-methyl-thiazol-2-yl]phenyl]azetidin-1-yl]methanone;
[6-(5-cyclopropyl-4H-1,2,4-triazol-3-yl)-2-azaspiro[3.3]heptan-2-yl]-[3-[4-(3,5-dimethyl-1,2,4-triazol-1-yl)phenyl]azetidin-1-yl]methanone;
[6-(5-cyclopropyl-4H-1,2,4-triazol-3-yl)-2-azaspiro[3.3]heptan-2-yl]-[4-[[6-(trifluoromethyl)-3-pyridyl]methyl]piperazino]methanone;
5-[[2-[6-(5-cyclopropyl-4H-1,2,4-triazol-3-yl)-2-azaspiro[3.3]heptane-2-carbonyl]-2-azaspiro[3.3]heptan-6-yl]methyl]-2-(trifluoromethyl)isonicotinonitrile;
[6-(3-cyclopropyl-1H-1,2,4-triazol-5-yl)-2-azaspiro[3.3]heptan-2-yl]-[6-[[3-(trifluoromethyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[fluoro-[6-(trifluoromethyl)pyridin-3-yl]methyl]-2-azaspiro[3.3]heptan-2-yl]methanone;
[6-[(5-cyclopropyl-3-methyl-pyrazol-1-yl)methyl]-2-azaspiro[3.3]heptan-2-yl]-[6-(3-cyclopropyl-1H-1,2,4-triazol-5-yl)-2-azaspiro[3.3]heptan-2-yl]methanone;
[6-[(3-cyclopropyl-5-methyl-pyrazol-1-yl)methyl]-2-azaspiro[3.3]heptan-2-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[3-(trifluoromethylsulfonimidoyl)phenoxy]-2-azaspiro[3.3]heptan-2-yl]methanone;
[6-(3-cyclopropyl-1H-1,2,4-triazol-5-yl)-2-azaspiro[3.3]heptan-2-yl]-[6-[2-fluoro-4-(trifluoromethylsulfonimidoyl)benzyl]-2-azaspiro[3.3]heptan-2-yl]methanone;
[6-[3-(trifluoromethyl)-1H-1,2,4-triazol-5-yl]-2-azaspiro[3.3]heptan-2-yl]-[6-(4-triflylbenzyl)-2,6-diazaspiro[3.3]heptan-2-yl]methanone:
[6-(5-cyclopropyl-4H-1,2,4-triazol-3-yl)-2-azaspiro[3.3]heptan-2-yl]-[3-[4-[3-(trifluoromethyl)-1H-pyrazol-5-yl]phenyl]azetidin-1-yl]methanone;
[6-[3-(1-aminocyclopropyl)-1H-1,2,4-triazol-5-yl]-2-azaspiro[3.3]heptan-2-yl]-[6-[2-fluoro-4-(trifluoromethyl)benzyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)-1H-pyrrolo[2,3-b]pyridin-6-yl]methyl]-2-azaspiro[3.3]heptan-2-yl]methanone;
5-[[2-[6-[3-(1-aminocyclopropyl)-1H-1,2,4-triazol-5-yl]-2-azaspiro[3.3]heptane-2-carbonyl]-2-azaspiro[3.3]heptan-6-yl]methyl]-2-(trifluoromethyl)benzonitrile;
3-[[2-[6-[3-(1-aminocyclopropyl)-1H-1,2,4-triazol-5-yl]-2-azaspiro[3.3]heptane-2-carbonyl]-2-azaspiro[3.3]heptan-6-yl]methyl]-5-(trifluoromethyl)benzonitrile;
[6-(3-cyclopropyl-1H-1,2,4-triazol-5-yl)-2-azaspiro[3.3]heptan-2-yl]-[6-[[1-methyl-3-(trifluoromethyl)pyrrolo[2,3-b]pyridin-6-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[4-(trifluoromethoxy)benzyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-(2-hydroxyethyl)-3-(trifluoromethyl)pyrrolo[2,3-b]pyridin-6-yl]methyl]-2-azaspiro[3.3]heptan-2-yl]methanone;
[6-(4-cyclopropylsulfonylbenzyl)-2-azaspiro[3.3]heptan-2-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[[4-[1-(trifluoromethyl)cyclopropyl]-imidazol-1-yl]methyl]-2-azaspiro[3.3]heptan-2-yl]methanone;
4-[[1-[6-(5-cyclopropyl-4H-1,2,4-triazol-3-yl)-2-azaspiro[3.3]heptane-2-carbonyl]-4-piperidyl]oxymethyl]-3-methoxy-benzonitrile;
[6-(5-cyclopropyl-4H-1,2,4-triazol-3-yl)-2-azaspiro[3.3]heptan-2-yl]-[6-[[5-[1-(trifluoromethyl)cyclopropyl]-1,3,4-oxadiazol-2-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[(5-dimethylphosphoryl-2-pyridyl)methyl]-2-azaspiro[3.3]heptan-2-yl]methanone;
[4-[5-chloro-1-[(2S)-3,3,3-trifluoro-2-hydroxy-propyl]indazol-3-yl]piperidino]-[6-(5-cyclopropyl-4H-1,2,4-triazol-3-yl)-2-azaspiro[3.3]heptan-2-yl]methanone;
[4-[5-chloro-1-[(2R)-3,3,3-trifluoro-2-hydroxy-propyl]indazol-3-yl]piperidino]-[6-(5-cyclopropyl-4H-1,2,4-triazol-3-yl)-2-azaspiro[3.3]heptan-2-yl]methanone;
[6-(3-cyclopropyl-1H-1,2,4-triazol-5-yl)-2-azaspiro[3.3]heptan-2-yl]-[3-[3-[[6-(trifluoromethyl)-3-pyridyl]methyl]-1-bicyclo[1.1.1]pentanyl]azetidin-1-yl]methanone;
3-[[2-[6-(3-cyclopropyl-1H-1,2,4-triazol-5-yl)-2-azaspiro[3.3]heptane-2-carbonyl]-2-azaspiro[3.3]heptan-6-yl]methyl]-5-(trifluoromethyl)benzonitrile;
[7-(4-chloro-2-mesyl-phenyl)-2,7-diazaspiro[3.5]nonan-2-yl]-[6-(5-cyclopropyl-4H-1,2,4-triazol-3-yl)-2-azaspiro[3.3]heptan-2-yl]methanone;
[4-[6-(5-cyclopropyl-4H-1,2,4-triazol-3-yl)-2-azaspiro[3.3]heptane-2-carbonyl]piperazino]-(4-ethylphenyl)methanone;
[4-[6-(5-cyclopropyl-4H-1,2,4-triazol-3-yl)-2-azaspiro[3.3]heptane-2-carbonyl]piperazino]-[5-(2-pyridyl)-2-thienyl]methanone;
[4-[6-(5-cyclopropyl-4H-1,2,4-triazol-3-yl)-2-azaspiro[3.3]heptane-2-carbonyl]piperazino]-[4-(methylamino)phenyl]methanone;
[4-[6-(5-cyclopropyl-4H-1,2,4-triazol-3-yl)-2-azaspiro[3.3]heptane-2-carbonyl]piperazino]-(4-methyl-2-thienyl)methanone;
[6-(5-cyclopropyl-4H-1,2,4-triazol-3-yl)-2-azaspiro[3.3]heptan-2-yl]-[3-[4-[3-(trifluoromethyl)pyrazol-1-yl]phenyl]azetidin-1-yl]methanone;
[6-(5-cyclopropyl-4H-1,2,4-triazol-3-yl)-2-azaspiro[3.3]heptan-2-yl]-[6-[[4-methyl-3-(trifluoromethyl)-1H-pyrazol-5-yl]methyl]-2,6-diazaspiro[3.3]heptan-2-yl]methanone;
[3-[4-[3-cyclopropyl-5-(difluoromethyl)pyrazol-1-yl]phenyl]azetidin-1-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[4-[[5-(trifluoromethyl)-2-pyridyl]methyl]piperazino]methanone;
[6-(5-cyclopropyl-4H-1,2,4-triazol-3-yl)-2-azaspiro[3.3]heptan-2-yl]-[6-[(2,4-dimethylthiazol-5-yl)methyl]-2,6-diazaspiro[3.3]heptan-2-yl]methanone;
[6-[(5-chloro-1,3-dimethyl-pyrazol-4-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[4-[(2-methyloxazol-4-yl)methyl]piperazino]methanone;
[6-(3-cyclopropyl-1H-1,2,4-triazol-5-yl)-2-azaspiro[3.3]heptan-2-yl]-[4-[(4-methylthiazol-5-yl)methyl]piperazino]methanone;
[4-[(5-chloro-1,3-dimethyl-pyrazol-4-yl)methyl]piperazino]-[6-(3-cyclopropyl-1H-1,2,4-triazol-5-yl)-2-azaspiro[3.3]heptan-2-yl]methanone;
4-[[2-[6-(3-cyclopropyl-1H-1,2,4-triazol-5-yl)-2-azaspiro[3.3]heptane-2-carbonyl]-2,6-diazaspiro[3.3]heptan-6-yl]methyl]-3-(trifluoromethyl)benzonitrile;
[6-(3-cyclopropyl-1H-1,2,4-triazol-5-yl)-2-azaspiro[3.3]heptan-2-yl]-[6-[[5-(difluoromethyl)-3-methyl-pyrazol-1-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[3-dimethylphosphoryl-5-(trifluoromethyl)benzyl]-2-azaspiro[3.3]heptan-2-yl]methanone;
4-[[1-[6-(5-cyclopropyl-4H-1,2,4-triazol-3-yl)-2-azaspiro[3.3]heptane-2-carbonyl]-4-piperidyl]oxymethyl]-3-methoxy-benzamide;
[6-[(3-tert-butyl-1,2,4-oxadiazol-5-yl)methyl]-2,6-diazaspiro[3.3]heptan-2-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[6-(pyrazolo[1,5-a]pyridin-3-ylmethyl)-2,6-diazaspiro[3.3]heptan-2-yl]methanone;
[6-(5-cyclopropyl-4H-1,2,4-triazol-3-yl)-2-azaspiro[3.3]heptan-2-yl]-[6-[(3-phenyl-1,2,4-oxadiazol-5-yl)methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[2-mesyl-4-(trifluoromethyl)benzyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[4-dimethylphosphoryl-2-(trifluoromethyl)benzyl]-2-azaspiro[3.3]heptan-2-yl]methanone;
[3-[(4-chloro-2-methoxy-benzyl)-methyl-amino]azetidin-1-yl]-[6-(5-cyclopropyl-4H-1,2,4-triazol-3-yl)-2-azaspiro[3.3]heptan-2-yl]methanone;
[2-(4-chloro-2-mesyl-benzyl)-6-azaspiro[3.4]octan-6-yl]-[6-(5-cyclopropyl-4H-1,2,4-triazol-3-yl)-2-azaspiro[3.3]heptan-2-yl]methanone;
[4-[(4-chloro-2-methoxy-benzyl)-methyl-amino]piperidino]-[6-(5-cyclopropyl-4H-1,2,4-triazol-3-yl)-2-azaspiro[3.3]heptan-2-yl]methanone;
[3-[[(4-chloro-2-methoxy-benzyl)-methyl-amino]methyl]azetidin-1-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[6-[(4-methylthiazol-2-yl)methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[(2-methylthiazol-4-yl)methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[(1,5-dimethylpyrazol-4-yl)methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[(2,4-dimethyloxazol-5-yl)methyl]-2,6-diazaspiro[3.3]heptan-2-yl]methanone;
5-chloro-2-[[1-[6-(3-cyclopropyl-1H-1,2,4-triazol-5-yl)-2-azaspiro[3.3]heptane-2-carbonyl]-4-piperidyl]methoxy]nicotinamide;
[6-[(3-cyclopropyl-1H-pyrazol-5-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[(2-methylpyrazol-3-yl)methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[(1-methylpyrazol-3-yl)methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[4-[[6-(trifluoromethyl)-3-pyridyl]oxymethyl]piperidino]methanone;
4-[[1-[6-(3-cyclopropyl-1H-1,2,4-triazol-5-yl)-2-azaspiro[3.3]heptane-2-carbonyl]-4-piperidyl]oxymethyl]-2-methoxy-benzonitrile;
[6-(3-cyclopropyl-1H-1,2,4-triazol-5-yl)-2-azaspiro[3.3]heptan-2-yl]-[4-[2-fluoro-4-(trifluoromethoxy)benzyl]oxypiperidino]methanone;
[6-(3-cyclopropyl-1H-1,2,4-triazol-5-yl)-2-azaspiro[3.3]heptan-2-yl]-[4-[2-fluoro-4-(trifluoromethyl)benzyl]oxypiperidino]methanone;
[6-(3-cyclopropyl-1H-1,2,4-triazol-5-yl)-2-azaspiro[3.3]heptan-2-yl]-[4-[2-mesyl-4-(trifluoromethyl)benzyl]-oxypiperidino]methanone;
5-chloro-2-[[1-[6-(3-cyclopropyl-1H-1,2,4-triazol-5-yl)-2-azaspiro[3.3]heptane-2-carbonyl]-4-piperidyl]methoxy]nicotinonitrile;
[6-(3-cyclopropyl-1H-1,2,4-triazol-5-yl)-2-azaspiro[3.3]heptan-2-yl]-[3-[3-(2-methylsulfonylphenyl)-1-bicyclo[1.1.1]pentanyl]azetidin-1-yl]methanone;
4-[4-[1-[6-(5-cyclopropyl-4H-1,2,4-triazol-3-yl)-2-azaspiro[3.3]heptane-2-carbonyl]azetidin-3-yl]phenyl]-1H-triazole-5-carbonitrile;
4-[[2-[6-(3-cyclopropyl-1H-1,2,4-triazol-5-yl)-2-azaspiro[3.3]heptane-2-carbonyl]-2-azaspiro[3.3]heptan-6-yl]methyl]-2-(trifluoromethoxy)benzoic acid methyl ester;
5-[[2-[6-(3-cyclopropyl-1H-1,2,4-triazol-5-yl)-2-azaspiro[3.3]heptane-2-carbonyl]-2-azaspiro[3.3]heptan-6-yl]methyl]-2-(trifluoromethoxy)benzoic acid methyl ester;
4-[[2-[6-(3-cyclopropyl-1H-1,2,4-triazol-5-yl)-2-azaspiro[3.3]heptane-2-carbonyl]-2-azaspiro[3.3]heptan-6-yl]methyl]-2-(trifluoromethoxy)benzamide;
5-[[2-[6-(3-cyclopropyl-1H-1,2,4-triazol-5-yl)-2-azaspiro[3.3]heptane-2-carbonyl]-2-azaspiro[3.3]heptan-6-yl]methyl]-2-(trifluoromethoxy)benzamide;
[6-(3-cyclopropyl-1H-1,2,4-triazol-5-yl)-2-azaspiro[3.3]heptan-2-yl]-[4-[[2-mesyl-4-(trifluoromethyl)phenoxy]-methyl]piperidino]methanone;
[4-[[4-fluoro-3-(trifluoromethyl)phenoxy]methyl]piperidino]-[6-[3-(1-hydroxycyclopropyl)-1H-1,2,4-triazol-5-yl]-2-azaspiro[3.3]heptan-2-yl]methanone;
[6-(5-cyclopropyl-4H-1,2,4-triazol-3-yl)-2-azaspiro[3.3]heptan-2-yl]-[4-[[2-mesyl-4-(trifluoromethoxy)phenoxy]methyl]piperidino]methanone;
[4-(2-chloro-4-mesyl-benzyl)oxypiperidino]-[6-[3-(1-hydroxycyclopropyl)-1H-1,2,4-triazol-5-yl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[4-[[2-mesyl-4-(trifluoromethyl)phenoxy]methyl]-piperidino]methanone;
[6-[3-(1-hydroxycyclopropyl)-1H-1,2,4-triazol-5-yl]-2-azaspiro[3.3]heptan-2-yl]-[6-(4-triflylbenzyl)-2,6-diazaspiro[3.3]heptan-2-yl]methanone;
[4-[[3-fluoro-5-(trifluoromethyl)phenoxy]methyl]piperidino]-[6-[3-(1-hydroxycyclopropyl)-1H-1,2,4-triazol-5-yl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[4-[[4-mesyl-3-(trifluoromethyl)phenoxy]-methyl]piperidino]methanone;
[6-[3-(1-hydroxycyclopropyl)-1H-1,2,4-triazol-5-yl]-2-azaspiro[3.3]heptan-2-yl]-[4-[[2-mesyl-4-(trifluoromethoxy)phenoxy]methyl]-piperidino]methanone;
[6-(5-cyclopropyl-1H-1,2,4-triazol-3-yl)-2-azaspiro[3.3]heptan-2-yl]-[4-[[4-mesyl-3-(trifluoromethyl)phenoxy]-methyl]piperidino]methanone;
[4-(2-chloro-4-mesyl-benzyl)oxypiperidino]-[6-(5-cyclopropyl-1H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[6-(4-dimethylphosphorylbenzyl)-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[[5-(trifluoromethyl)-2-pyridyl]methyl]-2-azaspiro[3.4]octan-2-yl]methanone;
[3-[3-[(5-cyclopropyl-1,3,4-oxadiazol-2-yl)methyl]-1-bicyclo[1.1.1]pentanyl]azetidin-1-yl]-[6-(3-cyclopropyl-1H-1,2,4-triazol-5-yl)-2-azaspiro[3.3]heptan-2-yl]methanone;
[6-[(3,5-difluoro-2-pyridyl)methyl]-2-azaspiro[3.4]octan-2-yl]-[6-[3-(1-hydroxycyclopropyl)-1H-1,2,4-triazol-5-yl]-2-azaspiro[3.3]heptan-2-yl]methanone;
[6-(5-cyclopropyl-4H-1,2,4-triazol-3-yl)-2-azaspiro[3.3]heptan-2-yl]-[3-[4-[3-(trifluoromethyl)-1,2,4-triazol-1-yl]phenyl]azetidin-1-yl]methanone;
[6-(5-cyclopropyl-4H-1,2,4-triazol-3-yl)-2-azaspiro[3.3]heptan-2-yl]-[6-(4-fluorophenyl)-2-azaspiro[3.3]heptan-2-yl]methanone;
[2-(4-chloro-2-fluoro-phenyl)-6-azaspiro[3.4]octan-6-yl]-[6-(5-cyclopropyl-4H-1,2,4-triazol-3-yl)-2-azaspiro[3.3]heptan-2-yl]methanone;
[2-(4-chloro-2-fluoro-phenyl)-6-azaspiro[3.4]octan-6-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[3-[4-(3-cyclopropyl-1,2,4-triazol-1-yl)phenyl]azetidin-1-yl]methanone;
[6-(5-cyclopropyl-4H-1,2,4-triazol-3-yl)-2-azaspiro[3.3]heptan-2-yl]-[6-[(3-dimethylphosphorylphenyl)methyl]-2-azaspiro[3.3]heptan-2-yl]methanone;
[6-(5-cyclopropyl-4H-1,2,4-triazol-3-yl)-2-azaspiro[3.3]heptan-2-yl]-[6-(5-fluoro-3-pyridyl)-2,6-diazaspiro[3.3]heptan-2-yl]methanone;
[3-[6-(2-azaspiro[3.4]octan-2-yl)-3-pyridyl]azetidin-1-yl]-[6-[3-(1-hydroxycyclopropyl)-1H-1,2,4-triazol-5-yl]-2-azaspiro[3.3]heptan-2-yl]methanone;
[6-(5-cyclopropyl-4H-1,2,4-triazol-3-yl)-2-azaspiro[3.3]heptan-2-yl]-[6-[2-fluoro-4-(trifluoromethyl)phenyl]-2-azaspiro[3.3]heptan-2-yl]methanone;
[6-(5-cyclopropyl-4H-1,2,4-triazol-3-yl)-2-azaspiro[3.3]heptan-2-yl]-[6-(4-mesylphenyl)-2-azaspiro[3.3]heptan-2-yl]methanone;
[6-(5-cyclopropyl-4H-1,2,4-triazol-3-yl)-2-azaspiro[3.3]heptan-2-yl]-[6-[4-(trifluoromethyl)phenyl]-2-azaspiro[3.3]heptan-2-yl]methanone;
[6-(3-cyclopropyl-1H-1,2,4-triazol-5-yl)-2-azaspiro[3.3]heptan-2-yl]-[3-[3-[[5-(trifluoromethyl)-2-pyridyl]oxymethyl]-1-bicyclo[1.1.1]pentanyl]azetidin-1-yl]methanone;
[6-(3-cyclopropyl-1H-1,2,4-triazol-5-yl)-2-azaspiro[3.3]heptan-2-yl]-[3-[3-[[4-(trifluoromethyl)-2-pyridyl]oxymethyl]-1-bicyclo[1.1.1]pentanyl]azetidin-1-yl]methanone;
[6-(3-cyclopropyl-1H-1,2,4-triazol-5-yl)-2-azaspiro[3.3]heptan-2-yl]-[6-[[3-methoxy-5-(trifluoromethyl)pyrazin-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[difluoro-[5-(trifluoromethyl)pyrazin-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-[1-(trifluoromethyl)cyclopropyl]-1H-pyrazol-3-yl]methyl]-2-azaspiro[3.3]heptan-2-yl]methanone;
1-[[3-[1-[6-(3-cyclopropyl-1H-1,2,4-triazol-5-yl)-2-azaspiro[3.3]heptane-2-carbonyl]azetidin-3-yl]-1-bicyclo[1.1.1]pentanyl]methyl]-5-(trifluoromethyl)-2-pyridone;
1-[[3-[1-[6-(3-cyclopropyl-1H-1,2,4-triazol-5-yl)-2-azaspiro[3.3]heptane-2-carbonyl]azetidin-3-yl]-1-bicyclo[1.1.1]pentanyl]methyl]-4-(trifluoromethyl)-2-pyridone;
[6-[3-(1-aminocyclopropyl)-1H-1,2,4-triazol-5-yl]-2-azaspiro[3.3]heptan-2-yl]-[6-[[5-(trifluoromethyl)-2-pyridyl]methyl]-2-azaspiro[3.3]heptan-2-yl]methanone;
[6-(3-cyclopropyl-1H-1,2,4-triazol-5-yl)-2-azaspiro[3.3]heptan-2-yl]-[3-[3-[[4-(trifluoromethyl)pyrazol-1-yl]methyl]-1-bicyclo[1.1.1]pentanyl]azetidin-1-yl]methanone;
[6-(3-cyclopropyl-1H-1,2,4-triazol-5-yl)-2-azaspiro[3.3]heptan-2-yl]-[6-[[4-(trifluoromethylsulfonimidoyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[(4-triflylpyrazol-1-yl)methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[4-(methylsulfonimidoyl)-3-(trifluoromethyl)benzyl]-2-azaspiro[3.3]heptan-2-yl]methanone;
[6-(3-cyclopropyl-1H-1,2,4-triazol-5-yl)-2-azaspiro[3.3]heptan-2-yl]-[6-[(4-mesylpyrazol-1-yl)methyl]-2-azaspiro[3.3]heptan-2-yl]methanone;
5-[[2-[6-(3-cyclopropyl-1H-1,2,4-triazol-5-yl)-2-azaspiro[3.3]heptane-2-carbonyl]-2-azaspiro[3.3]heptan-6-yl]methyl]-3-(difluoromethyl)-2-pyridone;
[6-(3-cyclopropyl-1H-1,2,4-triazol-5-yl)-2-azaspiro[3.3]heptan-2-yl]-[7-[[3-(trifluoromethyl)isoxazol-5-yl]methyl]-2,7-diazaspiro[3.5]nonan-2-yl]methanone;
3-[[2-[6-(3-cyclopropyl-1H-1,2,4-triazol-5-yl)-2-azaspiro[3.3]heptane-2-carbonyl]-2-azaspiro[3.3]heptan-6-yl]methyl]-5-(difluoromethyl)-2-pyridone;
[6-(3-cyclopropyl-1H-1,2,4-triazol-5-yl)-2-azaspiro[3.3]heptan-2-yl]-[6-[[5-(trifluoromethyl)-1,2,4-oxadiazol-3-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[4-methyl-5-(trifluoromethyl)-1,2,4-triazol-3-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[3-[1-(trifluoromethyl)cyclopropyl]-1,2,4-oxadiazol-5-yl]methyl]-2,6-diazaspiro[3.3]heptan-2-yl]methanone;
1-[[2-[6-(3-cyclopropyl-1H-1,2,4-triazol-5-yl)-2-azaspiro[3.3]heptane-2-carbonyl]-2-azaspiro[3.3]heptan-6-yl]methyl]-5-(trifluoromethyl)pyrazin-2-one;
[6-(3-cyclopropyl-1H-1,2,4-triazol-5-yl)-2-azaspiro[3.3]heptan-2-yl]-[6-[[2-(2,2,2-trifluoroethyl)triazol-4-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-(2,2,2-trifluoroethyl)-1,2,4-triazol-3-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[6-(2,2,2-trifluoroethoxy)-3-pyridyl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[5-[1-(trifluoromethyl)cyclopropyl]imidazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[6-isopropoxy-5-(trifluoromethyl)-2-pyridyl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[(1 S)-1-[4-methyl-5-(trifluoromethyl)-2-pyridyl]ethyl]-2-azaspiro[3.3]heptan-2-yl]methanone;
[6-(3-cyclopropyl-1 #H-1,2,4-triazol-5-yl)-2-azaspiro[3.3]heptan-2-yl]-[6-[(1 #R)-1-[4-methyl-5-(trifluoromethyl)pyridin-2-yl]ethyl]-2-azaspiro[3.3]heptan-2-yl]methanone;
[6-(3-cyclopropyl-1H-1,2,4-triazol-5-yl)-2-azaspiro[3.3]heptan-2-yl]-[3-[4-(1-triflylcyclopropyl)phenyl]azetidin-1-yl]methanone;
[6-(3-cyclopropyl-1H-1,2,4-triazol-5-yl)-2-azaspiro[3.3]heptan-2-yl]-[6-[[4-(trifluoromethyl)-2-pyridyl]oxy]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethyl)-2-pyridyl]oxymethyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-(2,2,2-trifluoroethyl)triazol-4-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-(3,3,3-trifluoropropyl)pyrazol-3-yl]methyl]-2,6-diazaspiro[3.3]heptan-2-yl]methanone;
[6-[(5-cyclopropylpyrazol-1-yl)methyl]-2-azaspiro[3.3]heptan-2-yl]-[6-(3-cyclopropyl-1H-1,2,4-triazol-5-yl)-2-azaspiro[3.3]heptan-2-yl]methanone;
[3-[3-[difluoro-[4-(trifluoromethyl)phenyl]methyl]-1-bicyclo[1.1.1]pentanyl]azetidin-1-yl]-[6-[3-(1-hydroxycyclopropyl)-1H-1,2,4-triazol-5-yl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[3-[3-(3-methyl-1H-pyrazol-5-yl)-1-bicyclo[1.1.1]pentanyl]azetidin-1-yl]methanone;
[6-[3-(3,3-difluorocyclobutyl)-1H-1,2,4-triazol-5-yl]-2-azaspiro[3.3]heptan-2-yl]-[6-[4-(methylsulfonimidoyl)benzyl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[(5-mesyl-2-pyridyl)methyl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[3-[2-[3-(trifluoromethyl)azetidin-1-yl]pyrimidin-5-yl]azetidin-1-yl]methanone;
[4-[6-(5-cyclopropyl-4H-1,2,4-triazol-3-yl)-2-azaspiro[3.3]heptane-2-carbonyl]piperazino]-(3-fluorophenyl)methanone;
4-[[1-[6-(3-cyclopropyl-1H-1,2,4-triazol-5-yl)-2-azaspiro[3.3]heptane-2-carbonyl]-4-piperidyl]oxymethyl]-2-methoxy-benzamide;
[3-[4-(2-chloro-4-mesyl-phenyl)phenyl]azetidin-1-yl]-[6-(l H-1,2,4-triazol-5-yl)-2-azaspiro[3.3]heptan-2-yl]methanone;
[6-[2-fluoro-4-(trifluoromethyl)phenyl]-2-azaspiro[3.3]heptan-2-yl]-[6-(1H-1,2,4-triazol-5-yl)-2-azaspiro[3.3]heptan-2-yl]methanone;
[6-(1H-1,2,4-triazol-5-yl)-2-azaspiro[3.3]heptan-2-yl]-[6-[[[1-(trifluoromethyl)cyclopropyl]amino]methyl]-2-azaspiro[3.3]heptan-2-yl]methanone;
[6-[4-mesyl-3-(trifluoromethyl)benzyl]-2-azaspiro[3.3]heptan-2-yl]-[6-(1H-1,2,4-triazol-5-yl)-2-azaspiro[3.3]heptan-2-yl]methanone;
[6-(1H-1,2,4-triazol-5-yl)-2-azaspiro[3.3]heptan-2-yl]-[6-[[4-(trifluoromethyl)-2-pyridyl]methyl]-2-azaspiro[3.3]heptan-2-yl]methanone;
[6-(1H-1,2,4-triazol-5-yl)-2-azaspiro[3.3]heptan-2-yl]-[6-[[6-(trifluoromethyl)-3-pyridyl]methyl]-2-azaspiro[3.3]heptan-2-yl]methanone;
[6-(1H-1,2,4-triazol-5-yl)-2-azaspiro[3.3]heptan-2-yl]-[6-[[5-(trifluoromethyl)-2-pyridyl]methyl]-2-azaspiro[3.3]heptan-2-yl]methanone;
[6-[(3,5-difluoro-2-pyridyl)methyl]-2-azaspiro[3.3]heptan-2-yl]-[6-(1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)-1H-indazol-6-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[2-(trifluoromethyl)imidazo[1,2-a]pyridin-6-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[2-(trifluoromethyl)imidazo[1,2-a]pyrazin-6-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)imidazo[1,5-a]pyridin-6-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[2-(trifluoromethyl)-[1,2,4]triazolo[1,5-a]pyridin-6-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)-1H-indazol-5-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)-[1,2,4]triazolo[4,3-a]pyridin-6-yl]methyl]-2-azaspiro[3.3]heptan-2-yl]methanone; and
[6-(3-cyclopropyl-1H-1,2,4-triazol-5-yl)-2-azaspiro[3.3]heptan-2-yl]-[6-[[2-(trifluoromethyl)pyrazolo[1,5-a]pyrimidin-6-yl]methyl]-2-azaspiro[3.3]heptan-2-yl]methanone.

In a preferred embodiment, the present invention provides a compound of formula (I) as described herein, or a pharmaceutically acceptable salt thereof, wherein said compound of formula (I) is selected from:
[6-(5-cyclopropyl-4H-1,2,4-triazol-3-yl)-2-azaspiro[3.3]heptan-2-yl]-[6-[[5-(trifluoromethyl)pyrazin-2-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[5-(trifluoromethyl)pyrimidin-2-yl]methyl]-2-azaspiro[3.3]heptan-2-yl]methanone;
[[6-(5-cyclopropyl-4H-1,2,4-triazol-3-yl)-2-azaspiro[3.3]heptan-2-yl]-[6-[[6-(trifluoromethyl)pyrimidin-4-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-(trifluoromethyl)pyrazol-4-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[4-(trifluoromethyl)pyrimidin-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)-1H-pyrazol-5-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-cyclopropyl-3-(trifluoromethyl)pyrazol-4-yl]methyl]-2-azaspiro[3.3]heptan-2-yl]methanone;
1-[[2-[6-(3-cyclopropyl-1H-1,2,4-triazol-5-yl)-2-azaspiro[3.3]heptane-2-carbonyl]-2-azaspiro[3.3]heptan-6-yl]methyl]-5-(trifluoromethyl)-2-pyridone;
[6-(3-cyclopropyl-1H-1,2,4-triazol-5-yl)-2-azaspiro[3.3]heptan-2-yl]-[6-[[5-(trifluoromethyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[4-(trifluoromethyl)triazol-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-(4-triflylbenzyl)-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[5-(trifluoromethyl)pyrazin-2-yl]methyl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-(4-triflylbenzyl)-2,6-diazaspiro[3.3]heptan-2-yl]methanone:
[6-(3-cyclopropyl-1H-1,2,4-triazol-5-yl)-2-azaspiro[3.3]heptan-2-yl]-[6-[[3-methyl-5-(trifluoromethyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-methyl-3-(trifluoromethyl)pyrazol-1-yl]methyl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[6-(trifluoromethyl)pyridazin-3-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[7-[[2-methyl-4-(trifluoromethyl)pyrazol-3-yl]methyl]-2,7-diazaspiro[3.5]nonan-2-yl]methanone;
[6-[3-(3,3-difluorocyclobutyl)-1H-1,2,4-triazol-5-yl]-2-azaspiro[3.3]heptan-2-yl]-[6-[[5-(trifluoromethyl)pyrazin-2-yl]methyl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[3-(2-fluoro-4-triflyl-benzyl)oxyazetidin-1-yl]methanone;
[6-[3-(1-hydroxycyclopropyl)-1H-1,2,4-triazol-5-yl]-2-azaspiro[3.3]heptan-2-yl]-[6-[[1-methyl-5-(trifluoromethyl)pyrazol-3-yl]methyl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[3-[5-[[1-(trifluoromethyl);
5-[[2-[6-[3-(1-fluorocyclopropyl)-1H-1,2,4-triazol-5-yl]-2-azaspiro[3.3]heptane-2-carbonyl]-2-azaspiro[3.3]heptan-6-yl]methyl]-3-(trifluoromethyl)picolinonitrile;
[6-[3-(1-fluorocyclopropyl)-1H-1,2,4-triazol-5-yl]-2-azaspiro[3.3]heptan-2-yl]-[6-[[5-(trifluoromethyl)-2-pyridyl]methyl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[4-(trifluoromethyl)pyrazol-1-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[3-[4-[3-(difluoromethyl)-5-methyl-1,2,4-triazol-1-yl]phenyl]azetidin-1-yl]methanone;
[6-(3-cyclopropyl-1H-1,2,4-triazol-5-yl)-2-azaspiro[3.3]heptan-2-yl]-[7-[[5-(trifluoromethyl)isoxazol-3-yl]methyl]-2,7-diazaspiro[3.5]nonan-2-yl]methanone;
[6-(3-cyclopropyl-1H-1,2,4-triazol-5-yl)-2-azaspiro[3.3]heptan-2-yl]-[7-(3-triflylbenzyl)-2,7-diazaspiro[3.4]octan-2-yl]methanone;
[6-[3-(1-fluorocyclopropyl)-1H-1,2,4-triazol-5-yl]-2-azaspiro[3.3]heptan-2-yl]-[3-[4-(trifluoromethylsulfonimidoyl)phenyl]azetidin-1-yl]methanone;
[6-[3-(1-fluorocyclopropyl)-1H-1,2,4-triazol-5-yl]-2-azaspiro[3.3]heptan-2-yl]-[6-[[2-(trifluoromethyl)pyrimidin-5-yl]methyl]-2-azaspiro[3.3]heptan-2-yl]methanone;
4-[[2-[6-[3-(1-fluorocyclopropyl)-1H-1,2,4-triazol-5-yl]-2-azaspiro[3.3]heptane-2-carbonyl]-2-azaspiro[3.3]heptan-6-yl]methyl]-2-(trifluoromethyl)benzonitrile:
[6-(3-cyclopropyl-1H-1,2,4-triazol-5-yl)-2-azaspiro[3.3]heptan-2-yl]-[3-[3-[5-(2,2-dimethylpropyl)-1,3,4-thiadiazol-2-yl]-1-bicyclo[1.1.1]-pentanyl]azetidin-1-yl]methanone;
5-[[2-[6-[3-(1-fluorocyclopropyl)-1H-1,2,4-triazol-5-yl]-2-azaspiro[3.3]heptane-2-carbonyl]-2-azaspiro[3.3]heptan-6-yl]methyl]-2-(trifluoromethyl)benzonitrile;
[6-[3-(1-fluorocyclopropyl)-1H-1,2,4-triazol-5-yl]-2-azaspiro[3.3]heptan-2-yl]-[6-[[1-methyl-5-(trifluoromethyl)pyrazol-3-yl]methyl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-(3-triflylbenzyl)-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[5-(trifluoromethyl)pyrimidin-2-yl]methyl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-[(5-triflyl-2-pyridyl)methyl]-2-azaspiro[3.3]heptan-2-yl]methanone;
[6-(3-cyclopropyl-1H-1,2,4-triazol-5-yl)-2-azaspiro[3.3]heptan-2-yl]-[6-(3-fluoro-5-triflyl-benzyl)-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)-1,2,4-oxadiazol-5-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[3-[3-[[5-(trifluoromethyl)pyrazin-2-yl]-amino]-1-bicyclo[1.1.1]-pentanyl]azetidin-1-yl]methanone;
[6-(3-cyclopropyl-1H-1,2,4-triazol-5-yl)-2-azaspiro[3.3]heptan-2-yl]-[6-[[4-[1-(trifluoromethyl)cyclopropyl]-pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[2-methyl-5-(trifluoromethyl)triazol-4-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[(5-triflyl-3-pyridyl)methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethylsulfonimidoyl)-2-pyridyl]methyl]-2-azaspiro[3.3]heptan-2-yl]methanone;
[6-[[5-(trifluoromethyl)pyrazin-2-yl]methyl]-2-azaspiro[3.3]heptan-2-yl]-[6-[3-(trifluoromethyl)-1H-1,2,4-triazol-5-yl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[5-[1-(trifluoromethyl);
[6-(3-cyclopropyl-1H-1,2,4-triazol-5-yl)-2-azaspiro[3.3]heptan-2-yl]-[6-[(5-triflyl-2-pyridyl)methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[difluoro-[6-(trifluoromethyl)-3-pyridyl]methyl]-2-azaspiro[3.3]heptan-2-yl]methanone;
5-[[2-[6-(5-cyclopropyl-4H-1,2,4-triazol-3-yl)-2-azaspiro[3.3]heptane-2-carbonyl]-2-azaspiro[3.3]heptan-6-yl]methyl]-2-(trifluoromethyl)nicotinonitrile;
5-[[2-[6-(5-cyclopropyl-4H-1,2,4-triazol-3-yl)-2-azaspiro[3.3]heptane-2-carbonyl]-2-azaspiro[3.3]heptan-6-yl]methyl]-2-(trifluoromethyl)isonicotinonitrile;
[6-(3-cyclopropyl-1H-1,2,4-triazol-5-yl)-2-azaspiro[3.3]heptan-2-yl]-[6-[[3-(trifluoromethyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[fluoro-[6-(trifluoromethyl)pyridin-3-yl]methyl]-2-azaspiro[3.3]heptan-2-yl]methanone;
[6-[(5-cyclopropyl-3-methyl-pyrazol-1-yl)methyl]-2-azaspiro[3.3]heptan-2-yl]-[6-(3-cyclopropyl-1H-1,2,4-triazol-5-yl)-2-azaspiro[3.3]heptan-2-yl]methanone;
[6-[(3-cyclopropyl-5-methyl-pyrazol-1-yl)methyl]-2-azaspiro[3.3]heptan-2-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[2-fluoro-4-(trifluoromethylsulfonimidoyl)benzyl]-2-azaspiro[3.3]heptan-2-yl]methanone;
[6-[3-(trifluoromethyl)-1H-1,2,4-triazol-5-yl]-2-azaspiro[3.3]heptan-2-yl]-[6-(4-triflylbenzyl)-2,6-diazaspiro[3.3]heptan-2-yl]methanone;
[6-(5-cyclopropyl-4H-1,2,4-triazol-3-yl)-2-azaspiro[3.3]heptan-2-yl]-[6-[[4-(trifluoromethyl)pyrazol-1-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[3-(trifluoromethyl)-1,2,4-thiadiazol-5-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[difluoro-[5-(trifluoromethyl)pyrazin-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-[1-(trifluoromethyl)cyclopropyl]-1H-pyrazol-3-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[(4-triflylpyrazol-1-yl)methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-[1-(trifluoromethyl)cyclopropyl]-1,2,4-oxadiazol-5-yl]methyl]-2,6-diazaspiro[3.3]heptan-2-yl]methanone;
1-[[2-[6-(3-cyclopropyl-1H-1,2,4-triazol-5-yl)-2-azaspiro[3.3]heptane-2-carbonyl]-2-azaspiro[3.3]heptan-6-yl]methyl]-5-(trifluoromethyl)pyrazin-2-one; and
[6-(3-cyclopropyl-1H-1,2,4-triazol-5-yl)-2-azaspiro[3.3]heptan-2-yl]-[6-[[1-(2,2,2-trifluoroethyl)-1,2,4-triazol-3-yl]methyl]-2-azaspiro[3.3]heptan-2-yl]methanone.

In a particularly preferred embodiment, the present invention provides a compound of formula (I) as described herein, or a pharmaceutically acceptable salt thereof, wherein said compound of formula (I) is selected from:
[6-(5-cyclopropyl-4H-1,2,4-triazol-3-yl)-2-azaspiro[3.3]heptan-2-yl]-[6-[[5-(trifluoromethyl)pyrazin-2-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[5-(trifluoromethyl)pyrimidin-2-yl]methyl]-2-azaspiro[3.3]heptan-2-yl]methanone;
[[6-(5-cyclopropyl-4H-1,2,4-triazol-3-yl)-2-azaspiro[3.3]heptan-2-yl]-[6-[[6-(trifluoromethyl)pyrimidin-4-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-(trifluoromethyl)pyrazol-4-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)-1H-pyrazol-5-yl]methyl]-2-azaspiro[3.3]heptan-2-yl]methanone;
1-[[2-[6-(3-cyclopropyl-1H-1,2,4-triazol-5-yl)-2-azaspiro[3.3]heptane-2-carbonyl]-2-azaspiro[3.3]heptan-6-yl]methyl]-5-(trifluoromethyl)-2 pyridone;
[6-(3-cyclopropyl-1H-1,2,4-triazol-5-yl)-2-azaspiro[3.3]heptan-2-yl]-[6-[[4-(trifluoromethyl)triazol-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)-1,2,4-oxadiazol-5-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[4-[1-(trifluoromethyl)cyclopropyl]-pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[(5-triflyl-3-pyridyl)methyl]-2-azaspiro[3.3]heptan-2-yl]methanone;
[6-[[5-(trifluoromethyl)pyrazin-2-yl]methyl]-2-azaspiro[3.3]heptan-2-yl]-[6-[3-(trifluoromethyl)-1H-1,2,4-triazol-5-yl]-2-azaspiro[3.3]heptan-2-yl]methanone;
[6-(5-cyclopropyl-4H-1,2,4-triazol-3-yl)-2-azaspiro[3.3]heptan-2-yl]-[6-[difluoro-[6-(trifluoromethyl)-3-pyridyl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)pyrazol-1-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[4-(trifluoromethyl)pyrazol-1-yl]methyl]-2-azaspiro[3.3]heptan-2-yl]methanone; and
[6-(5-cyclopropyl-4H-1,2,4-triazol-3-yl)-2-azaspiro[3.3]heptan-2-yl]-[6-[[3-(trifluoromethyl)-1,2,4-thiadiazol-5-yl]methyl]-2-azaspiro[3.3]heptan-2-yl]methanone.

In a particularly preferred embodiment, the present invention provides a compound of formula (I) as described herein, or a pharmaceutically acceptable salt thereof, wherein said compound of formula (I) is [6-(5-cyclopropyl-4H-1,2,4-triazol-3-yl)-2-azaspiro[3.3]heptan-2-yl]-[6-[[5-(trifluoromethyl)pyrazin-2-yl]methyl]-2-azaspiro[3.3]heptan-2-yl]methanone.

In a particularly preferred embodiment, the present invention provides a compound of formula (I) as described herein, or a pharmaceutically acceptable salt thereof, wherein said compound of formula (I) is [6-(5-cyclopropyl-4H-1,2,4-triazol-3-yl)-2-azaspiro[3.3]heptan-2-yl]-[6-[[5-(trifluoromethyl)pyrimidin-2-yl]methyl]-2-azaspiro[3.3]heptan-2-yl]methanone.

In a particularly preferred embodiment, the present invention provides a compound of formula (I) as described herein, or a pharmaceutically acceptable salt thereof, wherein said compound of formula (I) is [[6-(5-cyclopropyl-4H-1,2,4-triazol-3-yl)-2-azaspiro[3.3]heptan-2-yl]-[6-[[6-(trifluoromethyl)pyrimidin-4-yl]methyl]-2-azaspiro[3.3]heptan-2-yl]methanone.

In a particularly preferred embodiment, the present invention provides a compound of formula (I) as described herein, or a pharmaceutically acceptable salt thereof, wherein said compound of formula (I) is [6-(3-cyclopropyl-1H-1,2,4-triazol-5-yl)-2-azaspiro[3.3]heptan-2-yl]-[6-[[1-(trifluoromethyl)pyrazol-4-yl]methyl]-2-azaspiro[3.3]heptan-2-yl]methanone.

In a particularly preferred embodiment, the present invention provides a compound of formula (I) as described herein, or a pharmaceutically acceptable salt thereof, wherein said compound of formula (I) is [6-(3-cyclopropyl-1H-1,2,4-triazol-_5-yl)-2-azaspiro[3.3]heptan-2-yl]-[6-[[3-(trifluoromethyl)-1H-pyrazol-5-yl]methyl]-2-azaspiro[3.3]heptan-2-yl]methanone.

In a particularly preferred embodiment, the present invention provides a compound of formula (I) as described herein, or a pharmaceutically acceptable salt thereof, wherein said compound of formula (I) is 1-[[2-[6-(3-cyclopropyl-1H-1,2,4-triazol-5-yl)-2-azaspiro[3.3]heptane-2-carbonyl]-2-azaspiro[3.3]heptan-6-yl]methyl]-5-(trifluoromethyl)-2-pyridone.

In a particularly preferred embodiment, the present invention provides a compound of formula (I) as described herein, or a pharmaceutically acceptable salt thereof, wherein said compound of formula (I) is [6-(3-cyclopropyl-1H-1,2,4-triazol-5-yl)-2-azaspiro[3.3]heptan-2-yl]-[6-[[4-(trifluoromethyl)triazol-2-yl]methyl]-2-azaspiro[3.3]heptan-2-yl]methanone.

In a particularly preferred embodiment, the present invention provides a compound of formula (I) as described herein, or a pharmaceutically acceptable salt thereof, wherein said compound of formula (I) is [6-(3-cyclopropyl-1H-1,2,4-triazol-5-yl)-2-azaspiro[3.3]heptan-2-yl]-[6-[[3-(trifluoromethyl)-1,2,4-oxadiazol-5-yl]methyl]-2-azaspiro[3.3]heptan-2-yl]methanone.

In a particularly preferred embodiment, the present invention provides a compound of formula (I) as described herein, or a pharmaceutically acceptable salt thereof, wherein said compound of formula (I) is [6-(3-cyclopropyl-1H-1,2,4-triazol-5-yl)-2-azaspiro[3.3]heptan-2-yl]-[6-[[4-[1-(trifluoromethyl)cyclopropyl]-pyrazol-1-yl]methyl]-2-azaspiro[3.3]heptan-2-yl]methanone.

In a particularly preferred embodiment, the present invention provides a compound of formula (I) as described herein, or a pharmaceutically acceptable salt thereof, wherein said compound of formula (I) is [6-(3-cyclopropyl-1H-1,2,4-triazol-5-yl)-2-azaspiro[3.3]heptan-2-yl]-[6-[(5-triflyl-3-pyridyl)methyl]-2-azaspiro[3.3]heptan-2-yl]methanone.

In a particularly preferred embodiment, the present invention provides a compound of formula (I) as described herein, or a pharmaceutically acceptable salt thereof, wherein said compound of formula (I) is [6-[[5-(trifluoromethyl)pyrazin-2-yl]methyl]-2-azaspiro[3.3]heptan-2-yl]-[6-[3-(trifluoromethyl)-1H-1,2,4-triazol-5-yl]-2-azaspiro[3.3]heptan-2-yl]methanone.

In a particularly preferred embodiment, the present invention provides a compound of formula (I) as described herein, or a pharmaceutically acceptable salt thereof, wherein said compound of formula (I) is [6-(5-cyclopropyl-4H-1,2,4-triazol-3-yl)-2-azaspiro[3.3]heptan-2-yl]-[6-[difluoro-[6-(trifluoromethyl)-3-pyridyl]methyl]-2-azaspiro[3.3]heptan-2-yl]methanone.

In a particularly preferred embodiment, the present invention provides a compound of formula (I) as described herein, or a pharmaceutically acceptable salt thereof, wherein said compound of formula (I) is [6-(3-cyclopropyl-1H-1,2,4-triazol-5-yl)-2-azaspiro[3.3]heptan-2-yl]-[6-[[3-(trifluoromethyl)pyrazol-1-yl]methyl]-2-azaspiro[3.3]heptan-2-yl]methanone.

In a particularly preferred embodiment, the present invention provides a compound of formula (I) as described herein, or a pharmaceutically acceptable salt thereof, wherein said compound of formula (I) is [6-(5-cyclopropyl-4H-1,2,4-triazol-3-yl)-2-azaspiro[3.3]heptan-2-yl]-[6-[[4-(trifluoromethyl)pyrazol-1-yl]methyl]-2-azaspiro[3.3]heptan-2-yl]methanone.

In a particularly preferred embodiment, the present invention provides a compound of formula (I) as described herein, or a pharmaceutically acceptable salt thereof, wherein said compound of formula (I) is [6-(5-cyclopropyl-4H-1,2,4-triazol-3-yl)-2-azaspiro[3.3]heptan-2-yl]-[6-[[3-(trifluoromethyl)-1,2,4-thiadiazol-5-yl]methyl]-2-azaspiro[3.3]heptan-2-yl]methanone.

In a particular embodiment, the present invention provides pharmaceutically acceptable salts of the compounds according to formula (I) as described herein. In a further particular embodiment, the present invention provides compounds according to formula (I) as described herein as free bases.

In some embodiments, the compounds of formula (I) are isotopically-labeled by having one or more atoms therein replaced by an atom having a different atomic mass or mass number. Such isotopically-labeled (i.e., radiolabeled) compounds of formula (I) are considered to be within the scope of this disclosure. Examples of isotopes that can be incorporated into the compounds of formula (I) include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O, ¹⁷O, ¹⁸O, ³¹p, ³²P, ³⁵S, ¹⁸F, ³⁶Cl, ¹²³I, and ¹²⁵I, respectively. Certain isotopically-labeled compounds of formula (I), for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of formula (I) can be enriched with 1, 2, 5, 10, 25, 50, 75, 90, 95, or 99 percent of a given isotope.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the Examples as set out below using an appropriate isotopically-labeled reagent in place of the non-labeled reagent previously employed.

### Processes of Manufacturing

The preparation of compounds of formula (I) of the present invention may be carried out in sequential or convergent synthetic routes. Syntheses of the invention are shown in the following general schemes. The skills required for carrying out the reaction and purification of the resulting products are known to those persons skilled in the art. The substituents and indices used in the following description of the processes have the significance given herein, unless indicated to the contrary.

If one of the starting materials, intermediates or compounds of formula (I) contain one or more functional groups which are not stable or are reactive under the reaction conditions of one or more reaction steps, appropriate protective groups (as described e.g., in “Protective Groups in Organic Chemistry” by T. W. Greene and P. G. M. Wutts, 5th Ed., 2014, John Wiley & Sons, N.Y.) can be introduced before the critical step applying methods well known in the art. Such protective groups can be removed at a later stage of the synthesis using standard methods described in the literature.

If starting materials or intermediates contain stereogenic centers, compounds of formula (I) can be obtained as mixtures of diastereomers or enantiomers, which can be separated by methods well known in the art e.g., chiral HPLC, chiral SFC or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent. It is equally possible to separate starting materials and intermediates containing stereogenic centers to afford diastereomerically/enantiomerically enriched starting materials and intermediates. Using such diastereomerically/enantiomerically enriched starting materials and intermediates in the synthesis of compounds of formula (I) will typically lead to the respective diastereomerically/enantiomerically enriched compounds of formula (I).

A person skilled in the art will acknowledge that in the synthesis of compounds of formula (I)—insofar not desired otherwise—an orthogonal protection group strategy” will be applied, allowing the cleavage of several protective groups one at a time each without affecting other protective groups in the molecule. The principle of orthogonal protection is well known in the art and has also been described in literature (e.g. Barany and R. B. Merrifield, J. Am. Chem. Soc. 1977, 99, 7363; H. Waldmann et al., Angew. Chem. In. Ed. Engl. 1996, 3S, 2056).

A person skilled in the art will acknowledge that the sequence of reactions may be varied depending on reactivity and nature of the intermediates.

In more detail, the compounds of formula (I) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 2nd Edition, Richard C. Larock John Wiley & Sons, New York, NY. 1999). It was found convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux. The time required for the reaction may also vary widely, depending on many factors, notably the reaction temperature and the nature of the reagents. However, a period of from 0.5 hours to several days will usually suffice to yield the described intermediates and compounds. The reaction sequence is not limited to the one displayed in the schemes, however, depending on the starting materials and their respective reactivity, the sequence of reaction steps can be freely altered.

If starting materials or intermediates are not commercially available or their synthesis not described in literature, they can be prepared in analogy to existing procedures for close analogues or as outlined in the experimental section.

The following abbreviations are used in the present text: AcOH=acetic acid, ACN=acetonitrile, Bn=benzyl, BINAP=(2,2′-bis(diphenylphosphino)-1,1′-binaphthyl), Boc=tert-butyloxycarbonyl, CAS RN=chemical abstracts registration number, Cbz=benzyloxycarbonyl, CDI=N,N′-Carbonyldiimidazole, Cs₂CO₃=cesium carbonate, CO=carbon monoxide, CuCl=copper(I) chloride, CuCN=copper(I) cyanide, Cu=copper(I) iodide, DABCO=1,4-Diazabicyclo[2.2.2]octane; triethylenediamine, DAST=(diethylamino)sulfur trifluoride, DBU=1,8-diazabicyclo[5,4,0]undec-7-ene, DEAD=diethyl azodicarboxylate, DIAD=diisopropyl azodicarboxylate, DIBAL-H=diisobutyl aluminium hydride, DMAP=4-dimethylaminopyridine, DME=dimethoxyethane, DMEDA=N,N′-dimethylethylenediamine, DMF=N,N-dimethylformamide, DIPEA=N,N-diisopropylethylamine, dppf=1,1 bis(diphenyl phosphino)ferrocene, EDC·HCl=N-(3-dimethylaminopropyl)-N′-ethylcarbodiimide hydrochloride, EI=electron impact, ESI=electrospray ionization, EtOAc=ethyl acetate, EtOH=ethanol, h=hour(s), FA=formic acid, H₂O=water, H₂SO₄=sulfuric acid, HATU=1-[bis(dimethylamino)methylene]-1H-1,2,3-triazolo[4,5-b]pyridinium-3-oxide hexafluorophosphate, HBTU=O-benzotriazole-N,N,N′,N′-tetramethyl-uronium-hexafluoro-phosphate, HCl=hydrogen chloride, HOBt=1-hydroxy-1H-benzotriazole; HPLC=high performance liquid chromatography, iPrMgCl=isopropylmagnesium chloride, I2=iodine, IPA=2-propanol, ISP=ion spray positive (mode), ISN=ion spray negative (mode), K₂CO₃=potassium carbonate, KHCO₃=potassium bicarbonate, KI=potassium iodide, KOH=potassium hydroxide, K₃PO₄=potassium phosphate tribasic, LiAlH₄ or LAH=lithium aluminium hydride, LiHMDS=lithium bis(trimethylsilyl)amide, LiOH=lithium hydroxide, mCPBA=meta-chloroperoxybenzoic acid, MgSO₄=magnesium sulfate, min=minute(s), mL=milliliter, MPLC=medium pressure liquid chromatography, MS=mass spectrum, MTBE=Methyl tert-butyl ether, nBuLi=n-butyllithium, NaBH₃CN=sodium cyanoborohydride, NaH=sodium hydride, NBS=N-bromosuccinimide, NaHCO₃=sodium hydrogen carbonate, NaNO₂=sodium nitrite, NaBH(OAc)₃=sodium triacetoxyborohydride, NaOH=sodium hydroxide, Na₂CO₃=sodium carbonate, Na₂SO₄=sodium sulfate, Na₂S₂O₃=sodium thiosulfate, NEt₃=triethylamine (TEA), NH₄Cl=ammonium chloride, NMP=N-methyl-2-pyrrolidone, OAc=Acetoxy, T3P=propylphosphonic anhydride, PE=petroleum ether, PG=protective group, Pd—C=palladium on activated carbon, PdCl2(dppf)-CH₂Cl₂=1,1′-bis(diphenylphosphino)ferrocene-palladium(II)dichloride dichloromethane complex, Pd₂(dba)₃=tris(dibenzylideneacetone)dipalladium(0), Pd(OAc)₂=palladium(II) acetate, Pd(OH)₂=palladium hydroxide, Pd(PPh₃)₄=tetrakis(triphenylphosphine)palladium(0), PE=petroleum ether, PMP=1,2,2,6,6-Pentamethylpiperidine, PTSA=p-toluenesulfonic acid, R=any group, RP=reverse phase, RT=room temperature, SFC=Supercritical Fluid Chromatography, S-PHOS=2-dicyclohexylphosphino-2′,6′-dimethoxybiphenyl, TBAI=tetra butyl ammonium iodine, TEA=triethylamine, TFA=trifluoroacetic acid, THE=tetrahydrofuran, TMEDA=N,N,N′,N′-tetramethylethylenediamine, TS-TPP=triphenylphospine—polymer bound, XPhos=2-Dicyclohexylphosphino-2′,4′,6′-triisopropylbiphenyl, ZnCl₂=zinc chloride, Hal=halogen, prep-TLC=preparative thin layer chromatography.

The present compounds of formula I can be prepared by reacting an activated intermediate of formula 2 with the nucleophilic spirocyclic amine 1 by heating in a solvent such as DMF or CH₃CN in the presence of a base such as DIPEA. (Scheme 1)

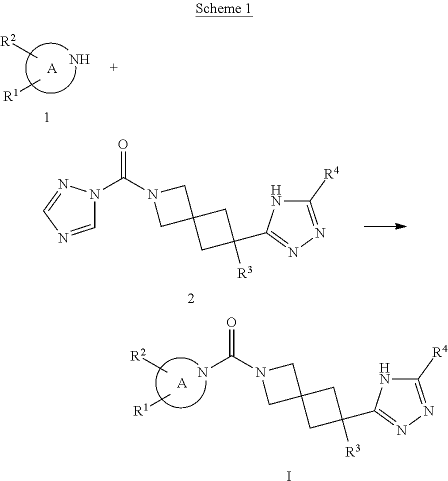

The activated intermediate 2 can be generated transiently in the reaction mixture, or by reacting an amine 3 with a coupling agent such as di(1H-1,2,4-triazol-1-yl)methanone in a solvent such as CH₂Cl₂ in the presence of a base such as DIPEA (Scheme 2).

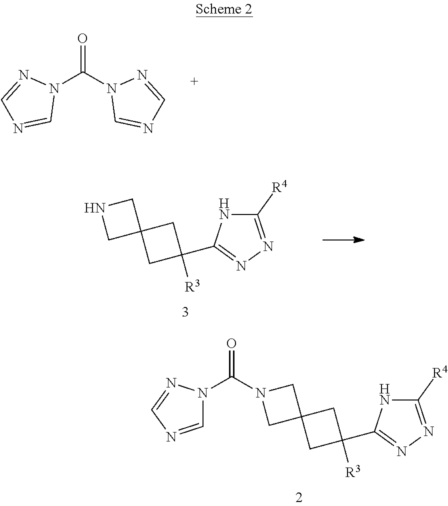

Building blocks of formula 4 where L₁=CH₂ can be generated by Suzuki reaction (e.g. (Pd(dppf)Cl₂, K₂CO₃, dioxane/H₂O), (X=Br, I) followed by hydrogenation (e.g. Pd/C, H₂). The required boronate intermediate 5 can be generated by reacting a ketone with 4,4,5,5-tetramethyl-2-[(tetramethyl-1,3,2-dioxaborolan-2-yl)methyl]-1,3,2-dioxaborolane (LiTMP, THF, −78° C.). (Scheme 3). Where B=N-linked heteroaryl, a Chan-Lam type coupling can be used in place of the Suzuki reaction (e.g. using Cu(OAc)₂ under oxygen atmosphere), followed by the hydrogenation/deprotection. A similar sequence could also be used to generate building blocks where e.g. L₁=—CH(Me)-, starting from a suitably functionalized boronate 5, with an additional Me group on the alkene carbon bearing the boronate.

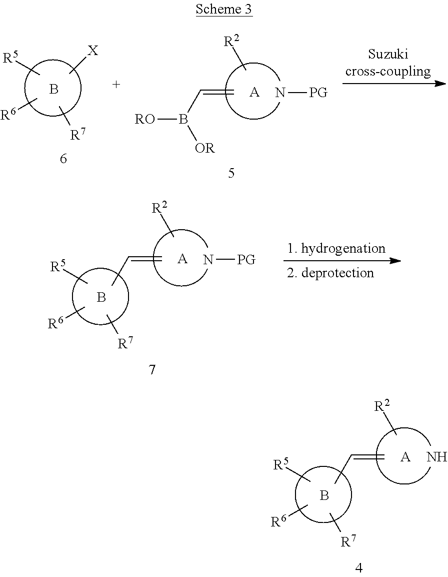

Building blocks of formula 8 with L₁=bond and B=C-linked (hetero)aryl can be generated by coupling a suitably protected boronic acid derivative 9 (X=B(OR)₂) with an iodide or bromide (Y=I or Br) 10 under nickel or palladium catalysis. Alternatively a bromide 9 (X=Br) can be coupled directly with 10 (Y=I or Br) in a photochemical reaction using Ir[dF(CF₃)ppy]₂(dtbbpy)PF₆, NiCl₂·DME, dtbbpy and (TMS)₃SiH. (Scheme 4) Alternatively the cross-coupling can be carried out under Negishi conditions with a zincate transiently generated from 10 (Y=I) and a (hetero)aryl halide (X=I, Br); or alternatively, the reaction of a p-tolylsulfonylhydrazono derivative of 10 and a boronic acid 9 (Y=B(OH)₂). Alternatively C-linked heteroaryl rings B may be installed using standard heterocyclic ring syntheses, typically starting from acid or cyano derivatives of the (spiro)cyclic amine A.

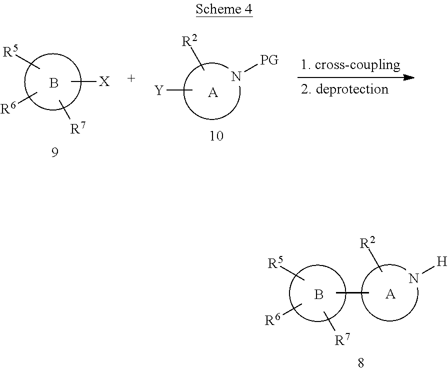

Alternatively, building blocks of formula 11 with L₁=oxygen and B is (hetero)aryl (where X is in a position suitable for SNAr displacement) can be prepared by reacting 12 (X is a leaving group such as Cl, Br, typically adjacent to aromatic N for SNAr reaction), with a suitably protected alcohol building block 13 in the presence of a base such as NaOtBu, followed by deprotection under standard conditions (e.g. with TsOH when PG=Boc). (Scheme 5). A similar scheme could be used for installing a small aliphatic unit or an aliphatic (hetero)cycle such as cyclopropyl as the B ring, via S_{N}2 displacement of a leaving group X (typically OMs, Br or 1) using a base such as NaH. Alternatively, building blocks of formula 11 with L₁=oxygen and B is (hetero)aryl can be generated by a palladium-catalyzed cross coupling of the alcohol 13 with the (hetero)aryl halide 12 (X=typically Br, I), followed by deprotection.

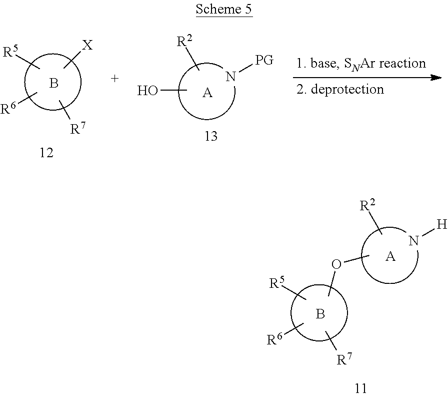

Alternatively, building blocks of formula 14 with L₁=NHC(O) could be prepared by reacting a suitably protected carboxylic acid 15 (or alternatively an acid chloride) with an amine 16 to produce amide 17 using standard amide coupling techniques (e.g. HATU, Et₃N), followed by deprotection. The amide 17 could also be reduced prior to deprotection (e.g. using borane-methyl sulfide complex) to yield amine building blocks of formula 18. (Scheme 6). These two sequences are also appropriate to install B rings when L₁=—CH₂NHCH₂— or —CH₂NHC(O)— starting from an amine building block 15 bearing a —CH₂NH₂ group in place of the amine.

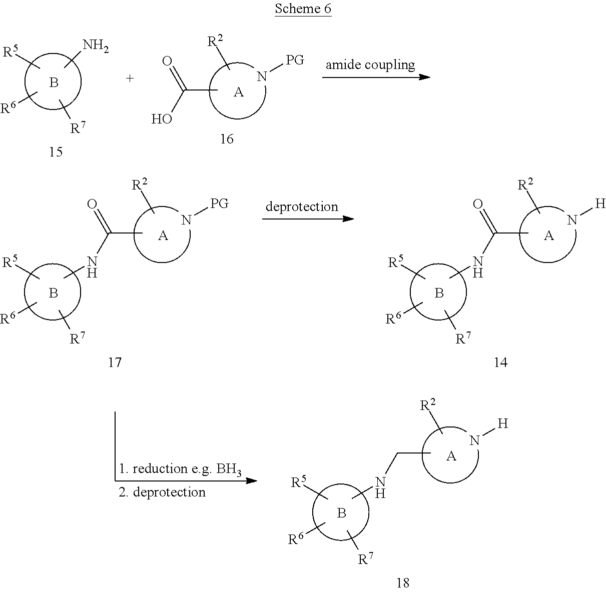

Alternatively, building blocks of formula 19 with L₁=SO₂NH can be prepared by reacting sulfonyl chloride 20 with a suitably protected amine building block 21 in the presence of a base such as DIPEA, followed by deprotection under standard conditions (e.g. with TsOH or TFA when PG=Boc). (Scheme 7)

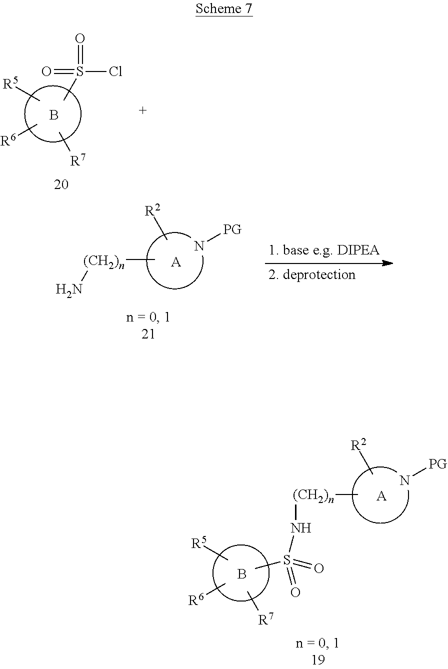

Alternatively, building blocks of formula 22 with L₁=NH or CH₂NH and B is (hetero)aryl (where X is in a position suitable for SNAr displacement) can be prepared by reacting 12 (X is a leaving group such as Cl, Br, often adjacent to aromatic N for SNAr reaction), with a suitably protected amine building block 21 in the presence of a base such as DIPEA, followed by deprotection under standard conditions (e.g. with TsOH when PG=Boc). (Scheme 8)

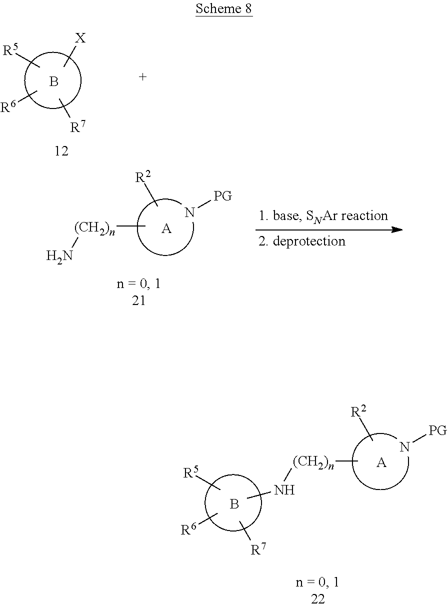

Alternatively, building blocks of formula 22 with L₁=bond and B is N-linked heterocyclic can be prepared by reacting nucleophilic heterocycle B (24), with a suitably protected building block 25 (X=leaving group such as OMs, I, Br) in the presence of a base such as Cs₂CO₃, followed by deprotection under standard conditions (e.g. with TsOH when PG=Boc). (Scheme 9) Typically mesylate building blocks 25 were used (X=OMs), which can conveniently be generated from the hydroxyl analog by reacting with MsCl in the presence of a mild base such as Et₃N.

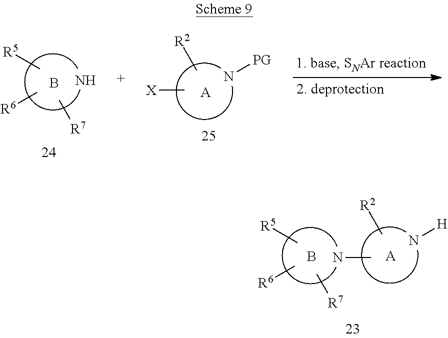

Alternatively, building blocks of formula 22 with L₁=bond and B is N-linked heterocyclic can be prepared by a reductive amination reaction of heterocycle B (24) with a suitably protected ketone building block 26 in the presence of a reducing agent such as sodium triacetoxyborohydride or sodium cyanoborohydride, followed by deprotection under standard conditions (e.g. with TsOH when PG=Boc). (Scheme 10)

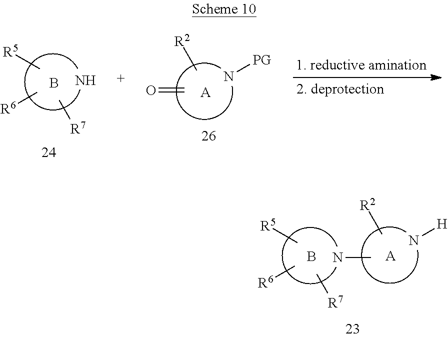

Alternatively, building blocks of formula 27 with L₁=CH₂N may be installed by a reductive amination reaction of amine 28 with a suitably protected ketone building block 26 in the presence of a reducing agent such as sodium triacetoxyborohydride or sodium cyanoborohydride, followed by deprotection under standard conditions (e.g. with TsOH when PG=Boc). (Scheme 11)

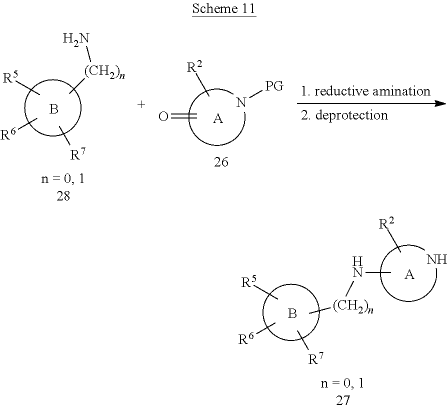

Alternatively, building blocks of formula 28 with L₁=CH₂ and A is N-linked can be prepared by a reductive amination reaction of aldehyde 29 with suitably protected heterocycle A (30) in the presence of a reducing agent such as sodium triacetoxyborohydride or sodium cyanoborohydride, followed by deprotection under standard conditions (e.g. with TsOH when PG=Boc). (Scheme 12) The same sequence can also be carried out to generate building blocks of formula 31 where with L₁=CH—NH or CH₂NHCH₂ from aldehyde 29 and amine 21. (Scheme 12) Alternatively building blocks 31a where L₁=CH₂NR^{13a} can be generated via reductive amination of an aldehyde 29 with a suitably protected alkylamine building block 21a. Alternatively building blocks 31a where L₁=CH₂NR^{13a} can be generated via reductive amination of an aldehyde 29 with a suitably protected amine building block 21a followed by alkylation of the amino group (e.g. using Cs₂CO₃, and a suitable alkylbalide R^{13a}—X where X=Br or I, in a solvent such as DMF), and deprotection. (Scheme 13).

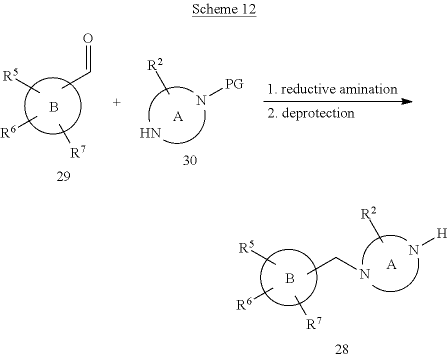

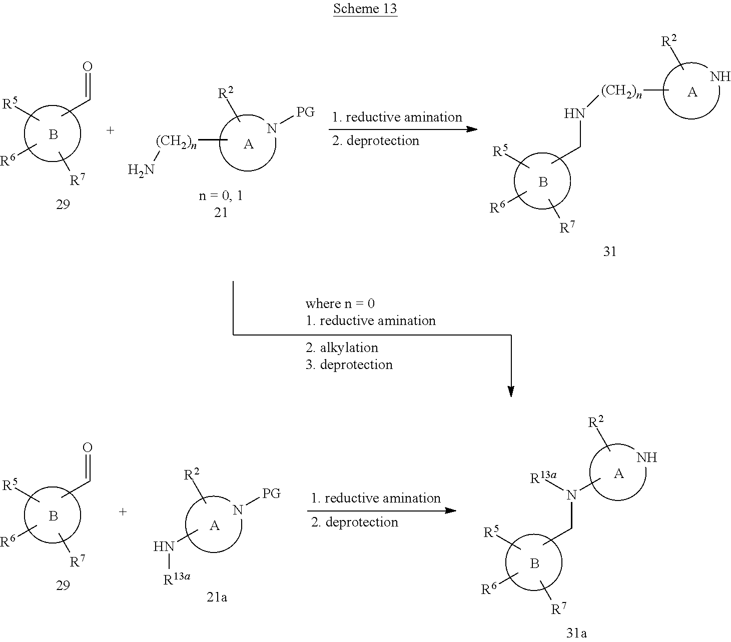

Alternatively, building blocks of formula 32 with L₁=SO₂ and A is N-linked can be prepared from a sulfonyl chloride 33 and suitably protected heterocycle A (30) in the presence of a base such as DIPEA, followed by deprotection under standard conditions (e.g. with TsOH when PG=Boc). (Scheme 14) This sequence is also appropriate where A is N-linked and R₁ is C₁-C₆-alkyl-SO₂—.

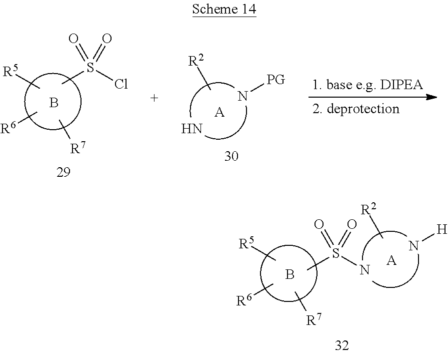

Alternatively, building blocks of formula 11 with L₁=oxygen and B is (hetero)aryl (where OH is in a position such that it is nucleophilic) can be prepared by reacting a suitably protected building block 13 (X is a leaving group such as Cl, Br, I or OMs), with alcohol 34 in the presence of a base such as NaH, followed by deprotection under standard conditions (e.g. with TsOH when PG=Boc). (Scheme 15) A similar procedure could be used for installing a small aliphatic unit in place of the B ring, or an aliphatic (hetero)cycle such as cyclopropyl as the B ring with an additional CH₂ linker, via S_{N}2 displacement of the leaving group X (typically OMs, Br or I) by a small aliphatic alcohol.

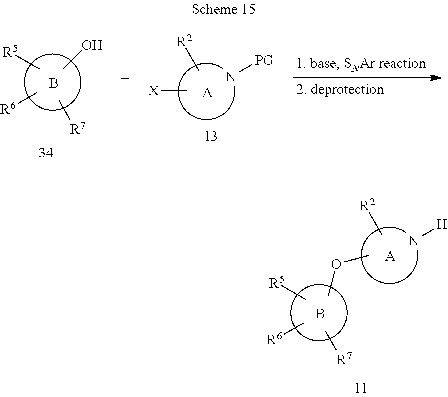

Alternatively, building blocks of formula 28 with L₁=CH₂ and A is N-linked can be prepared by a reductive amination reaction of (hetero)aryl methylhalide (X=Cl, Br, I) 35 with suitably protected heterocycle A (30) in the presence of a base such as K₂CO₃ in a solvent such as ACN, followed by deprotection under standard conditions (e.g. with TsOH when PG=Boc). (Scheme 16)

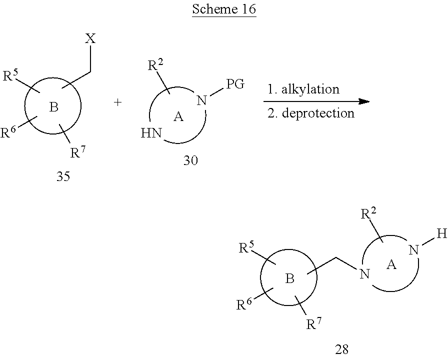

Alternatively, building blocks of formula 28 with L₁=CH₂ and A is N-linked can be prepared by a reductive amination reaction of acid chloride 36 with suitably protected heterocycle A (30) in the presense of a base (e.g. DIPEA) to form an amide, follow by reduction of the amide (e.g. using borane tetrahydrofuran complex), and deprotection under standard conditions (e.g. with TsOH when PG=Boc). (Scheme 17)

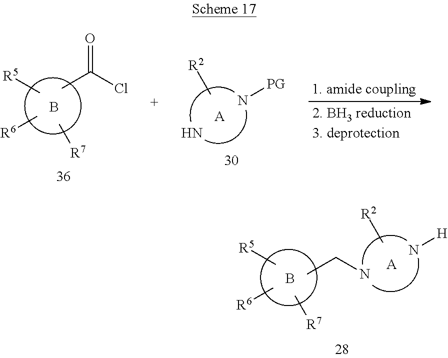

Alternatively, sulfonylurea building blocks of formula 37 with L₁=NHSO₂— or —CH₂NHSO₂—, can be prepared by activating 2-methyl-1-(2-methylimidazol-1-yl)sulfonyl-imidazole by methylation with methyl trifluoromethanesulfonate, followed by reaction with suitably protected amine 30; a further sequence of activation by methylation with methyl trifluoromethanesulfonate followed by reaction with amine 28; and finally deprotection under standard conditions (e.g. with TsOH or TFA when PG=Boc). (Scheme 18) Alternatively sulfonylurea building blocks 37 can be generated from sulfuryl chloride followed by sequential additions of 30 and 28 in the presence of a base such as Et₃N or DIPEA, and finally deprotection under standard conditions.

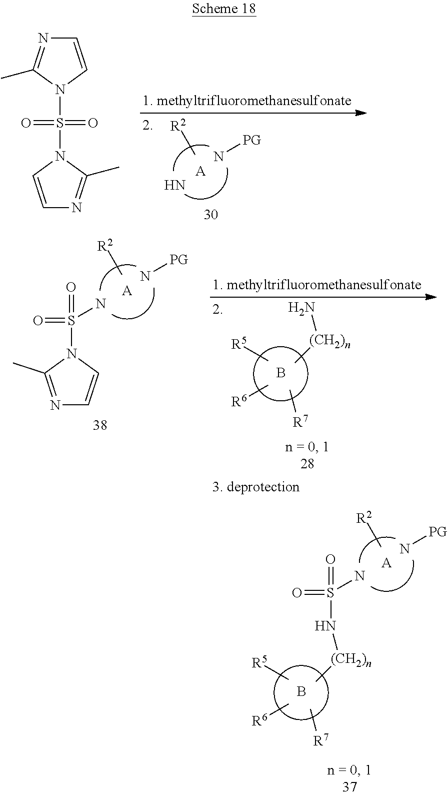

Alternatively, building blocks of formula 38 with L₁=bond, A is N-linked and B is (hetero)aryl can be prepared using a metal-catalysed cross-coupling reaction (e.g. Buchwald reaction, Pd-catalysis) between a suitably protected heterocycle A (30) and a (hetero)arylhalide (X=Br, I, Cl), followed by deprotection under standard conditions (e.g. with TsOH or TFA when PG=Boc). (Scheme 19) In certain cases, if the (hetero)aryl 39 is suitable for SNAr reaction (X=F) then building blocks of formula 38 can be generated by SNAr reaction of (spiro)cycle A 30 and 39, in the presence of a base (e.g. K₂CO₃ or Et₃N, DMSO, heating).

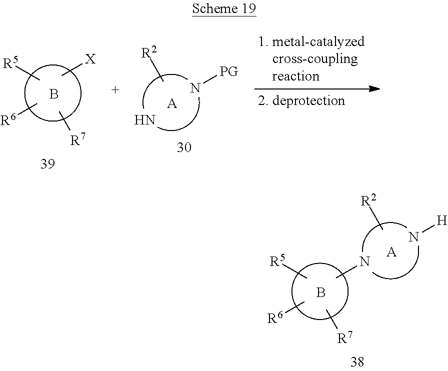

Alternatively, building blocks of formula 40 with L₁=bond and B is N-linked heterocyclic can be prepared by a reductive amination reaction of suitably protected aldehyde 41 with a heterocyclic amine 24 in the presence of a reducing agent such as sodium triacetoxyborohydride or sodium cyanoborohydride, followed by deprotection under standard conditions (e.g. with TsOH when PG=Boc). (Scheme 20)

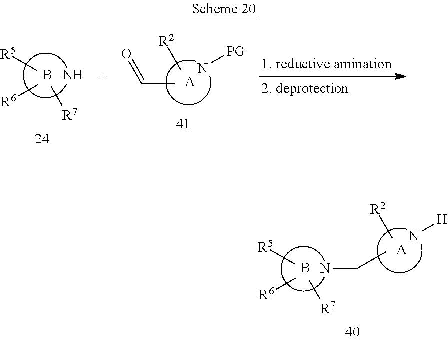

Alternatively, building blocks of formula 42 with L₁=C(O)NH could be prepared by reacting a suitably protected amine 44 with a carboxylic acid 43 to produce amide 45 using standard amide coupling techniques (e.g. HATU, DIPEA), followed by deprotection under standard conditions (e.g. with TsOH or TFA when PG=Boc). (Scheme 21).

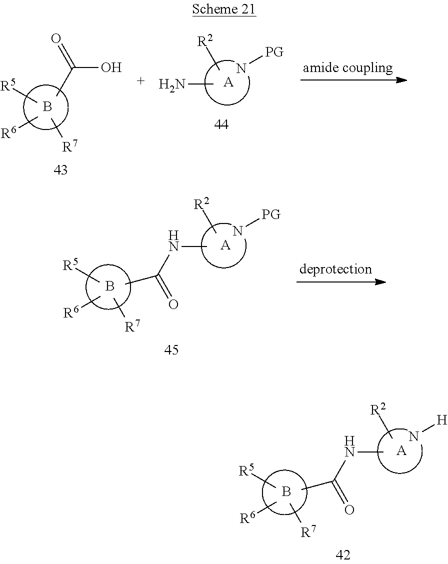

Alternatively, building blocks of formula 46, where L₁=bond, B=(hetero)aryl, L₂=bond and C=cyclopropyl can be generated by a metal-catalyzed cross coupling of a suitably protected halide 10 (Y=I, Br) with a cyclopropyl(hetero)aryl halide 49 (X=Br, Cl), such as photochemical reaction of 10 (Y=Br) with halide 49 using Ir[dF(CF₃)ppy]₂(dtbbpy)PF₆, NiCl₂·DME, dtbbpy and (TMS)₃SiH. Where R₉ and R₁₁=H, and R₁₀=CF₃, the trifluoromethylcyclopropyl building blocks 49 were not generally available, and were instead generated from halide building block 47 (X₁=I, Br needs to be more reactive to Suzuki reaction than X₂=typically Br, Cl adjacent to aromatic N) via Suzuki reaction with 1-(trifluoromethyl)vinylboronic acid to give 48. Cyclopropanation using diphenyl(methyl)sulfonium tetrafluoroborate and LiHMDS gave the required building block 49. (Scheme 22) Alternatively the order of the steps could be exchanged such that a Negishi cross-coupling of 10 (Y=I) was carried out with 47 first (in this case X₁ needs to be less reactive to cross-coupling than X₂) to yield intermediate 52 (Scheme 23), followed by the Suzuki-cyclopropanation sequence and deprotection.

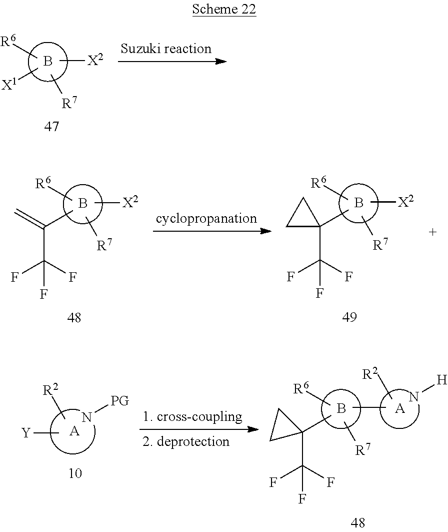

Building blocks with L₁=bond and B=C-linked (hetero)aryl and R₅=amine (L₂=—NH— or —CH₂NH— and C is an aliphatic heterocycle such as cyclopropyl (50) or C=N-linked heterocycle (51)) can be generated by metal-catalyzed amination of intermediate 52 (X₁=Br, Cl adjacent to aromatic N) with the relevant amine building block 53 or 54 (typically Pd-catalysis, Buchwald reaction). The required intermediate 52 could be generated by a cross-coupling such as Negishi reaction between a zincate transiently generated suitably protected building block 10 (Y=I) and a suitable dihalogenated (hetero)aryl building block 47 (X₂ needs to be more reactive to the cross coupling conditions than XI; typically X₂=I or Br, and X₁=Br). (Scheme 23) Alternatively the intermediate 52 can be generated by reacting a p-tolylsulfonylhydrazono derivative of 10 (Y==N—NH-Ts) with a boronic acid 47 (X₂=B(OH)₂) in the presence of a base such as K₂CO₃.

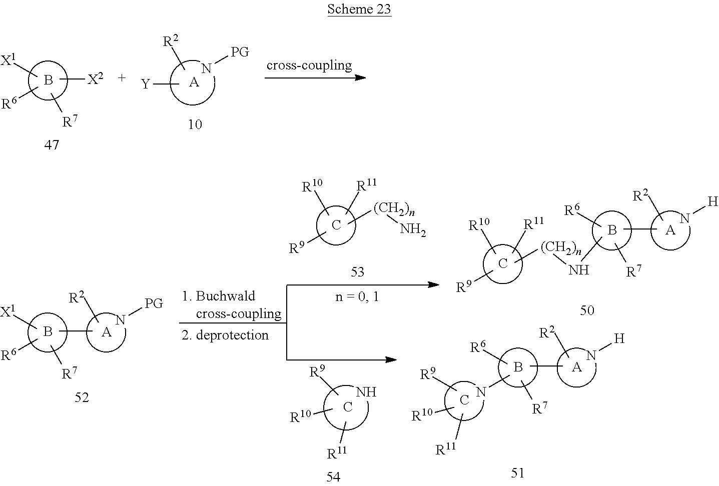

Alternatively, the amine 53 or 54 can be reacted with (hetero)aryl building block 47 (X₁=F) in the presence of a base (such as DIEA or K₂CO₃) in an SNAr reaction to generate intermediate 55 or 56, before carrying out the cross coupling reaction with the A ring (typically using photochemical reaction of 10 (Y=Br) with halide 55 or 56 using Ir[dF(CF₃)ppy]₂(dtbbpy)PF₆, NiCl₂·DME, dtbbpy and (TMS)₃SiH), followed by deprotection. (Scheme 23) Alternatively this SNAr approach to produce the R₅ amine could be carried out on intermediates such as 52 (where X₁=F). (Scheme 24) Occasionally copper-catalyzed SNAr or Ullmann type reactions were used for the reaction of 47 and 54 to give 56.

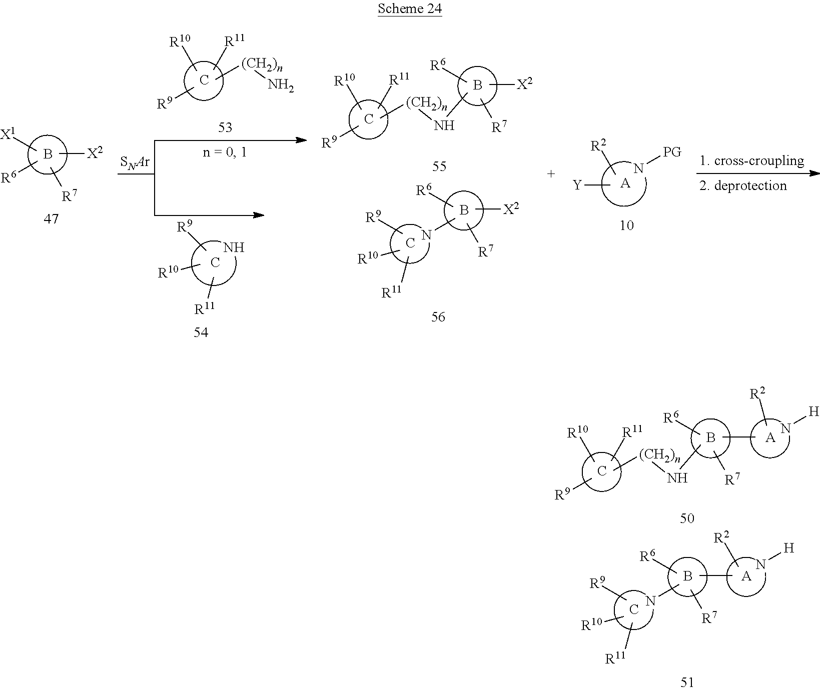

Building blocks with L₁=bond and B=C-linked (hetero)aryl and C=N-linked heteroaryl (57) can be generated by Chan-Lam coupling (in presence of Cu(OAc)₂) of a boronic acid 47 (X₁=B(OH)₂, X₂=Br) with the heteroaryl to give intermediate 56, which could then be reacted with suitably protected (spiro)cyclic amine 10 (Y=Br) in a photochemical cross-coupling using Ir[dF(CF₃)ppy]₂(dtbbpy)PF₆, NiCl₂·DME, dtbbpy and (TMS)₃SiH), followed by deprotection. (Scheme 25) Alternatively, for C-linked heterocycles, the heterocycle C can be constructed via standard heterocyclic synthesis techniques prior to the photochemical cross coupling reaction and deprotection. In some instances, the order of the steps can also be reversed.

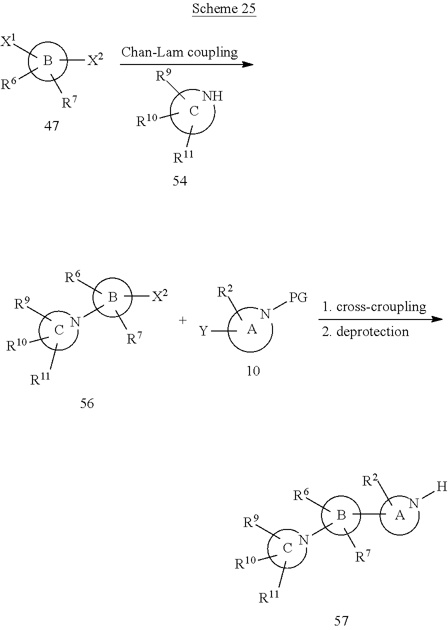

Building blocks (58) with L₁=—CH₂O— can be generated from a suitably protected hydroxylated building block 13 and a (hetero)aryl building block 35 bearing a leaving group in the benzylic position (X=typically Br), via alkylation (e.g. using potassium tertbutoxide) followed by a deprotection (e.g. with TsOH or TFA when PG=Boc). (Scheme 26) A similar approach can also be used for the installation of small alkyl units, bearing an aliphatic B ring.

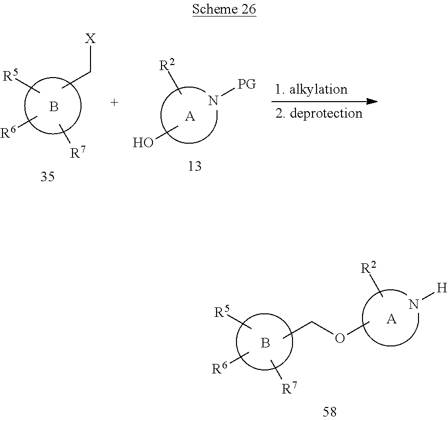

Alternatively, building blocks of formula 59 with L₁=oxygen and B is (hetero)aryl can be prepared by reacting a suitably protected (spiro)cyclic amine bearing a hydroxyl group (13) with a nucleophilic (hetero)aryl alcohol (60) under Mitsunobu-type conditions (e.g. using Tsunoda reagent, (tributylphosphoranylidene)acetonitrile or PPh₃/DIAD), followed by deprotection. (Scheme 27)

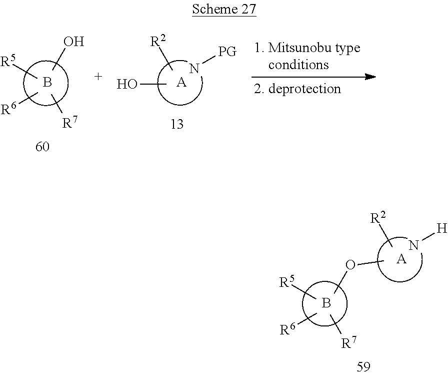

Alternatively, building blocks of formula 61 with L₁ and L₂=bond and B and C are (hetero)aryl can be prepared by reacting a suitably protected (spiro)cyclic amine (Y=I or Br) in two sequential cross coupling reactions, followed by deprotection. Most typically this involves a Ni-catalyzed cross-coupling between 10 (Y=I) and a building block 62 bearing both a bromide and boronic acid functionality to give bromo (hetero)aryl intermediate 63. Intermediate 63 could be coupled with a boronic acid derivative 64 under Suzuki conditions followed by deprotection to give 61. (Scheme 28)

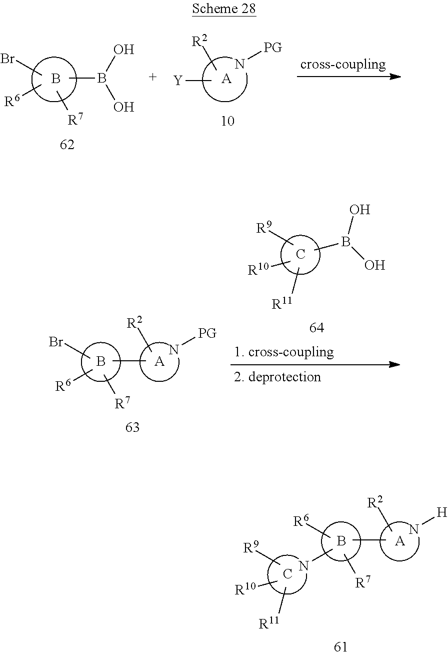

Building blocks of formula 65 where L₁=bond and L₂=—NHCH₂— can be generated by reductive amination reaction of suitably protected aldehyde 66 with an amine 53 in the presence of a reducing agent such as sodium triacetoxyborohydride or sodium cyanoborohydride, followed by deprotection under standard conditions (e.g. with TsOH when PG=Boc). In some instances, the reductive amination step can occur prior to the coupling of A and B, which can be done as described in Scheme 4 (Scheme 29)

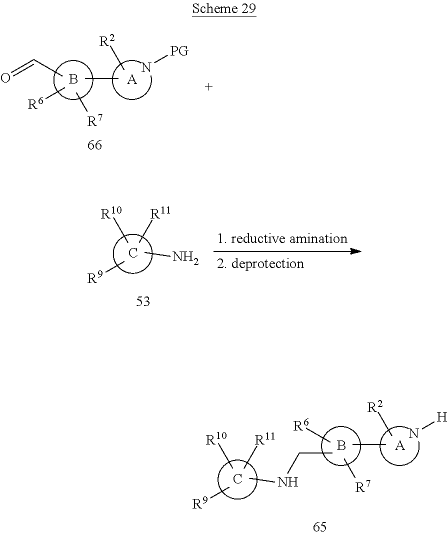

Building blocks of formula 67 where L₁=bond and L₂=—NHCH₂— can be generated by Curtius rearrangement of suitably protected acid building block 68 (e.g. using diphenylphosphonic azide, benzyl alcohol) and a deprotection of the generated carbamate (e.g. with Pd/C, H₂) to give amine 69, followed by further reductive amination of amine 69 with a suitable aldehyde 7U. and subsequent deprotection. (Scheme 3U)

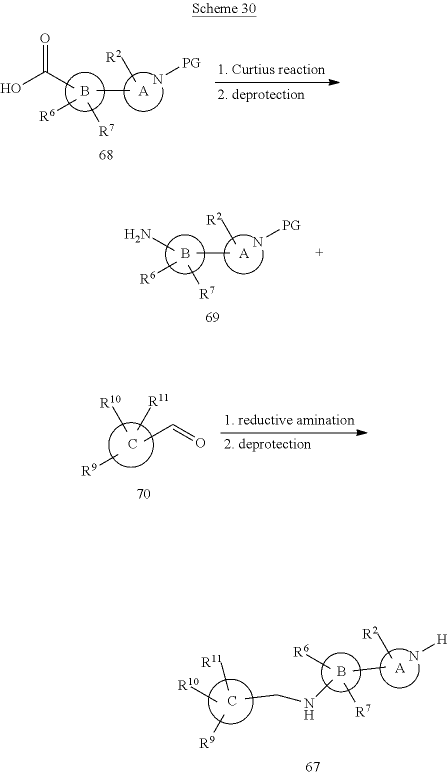

Building blocks of formula 71 where L₁=bond and L₂=bond and C=heteroaryl can be generated using standard heteroaryl synthesis techniques starting from a building block 72 bearing a cyano or carboxylic acid group (X=CN, COOH). Typical heterocyclic synthesis reactions can include e.g. reaction of a carboxylic acid with a hydrazide to generate a 1,3,4 oxadiazole derivative, reaction of a cyano group with a hydrazide derivative to generate a 1,2,4 triazole, cycloaddition of a cyano group with azidotrimethylsilane to yield a tetrazole. (Scheme 31)

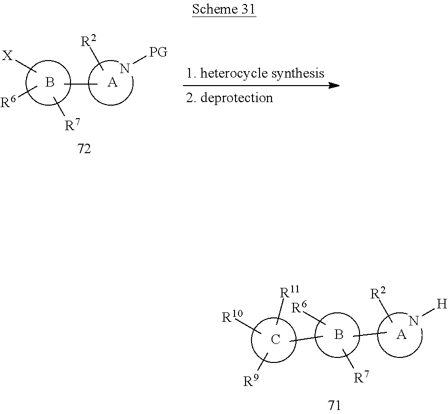

Alternatively, building blocks of formula 73 with L₁=—CH₂NH— or —CH₂NH— may be installed by a reductive amination reaction of amine 28 with a suitably protected aldehyde building block 41 in the presence of a reducing agent such as sodium triacetoxyborohydride or sodium cyanoborohydride, followed by deprotection under standard conditions (e.g. with TsOH when PG=Boc). (Scheme 32)

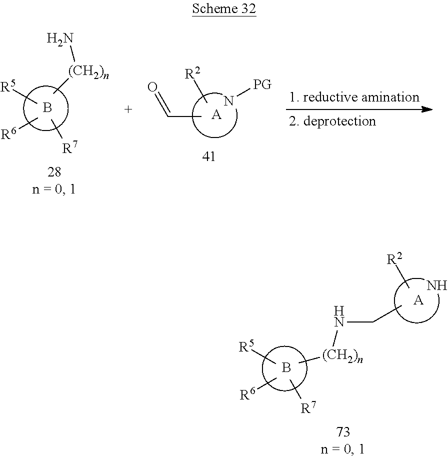

Alternatively, building blocks of formula 74 where A is C-linked and L₁=—SO₂— may be generated by S_{N}2 reaction of a thiol 75 with (spiro)cyclic amine A (Y=leaving group, such as OMs) under basic conditions (e.g. K₂CO₃) followed by oxidation of the thioether to the sulfone (e.g. with mCPBA) and deprotection under standard conditions (e.g. with TsOH when PG=Boc). (Scheme 33) The same sequence can be used to generate sulfoxime (L₁=—SNO—) using a modified oxidation step to install the sulfoximine from the thioether (e.g. using iodobenzene diacetate and ammonium carbamate).

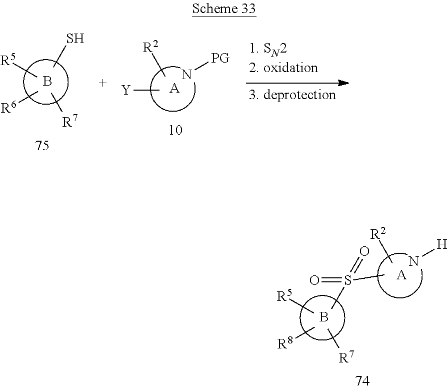

Alternatively, building blocks of formula 76 with L₁=—SO₂NH— can be prepared from a sulfonyl chloride 33 and suitably protected (spiro)cyclic amine (44) in the presence of a base such as DIPEA, followed by deprotection under standard conditions (e.g. with TsOH when PG=Boc). (Scheme 34)

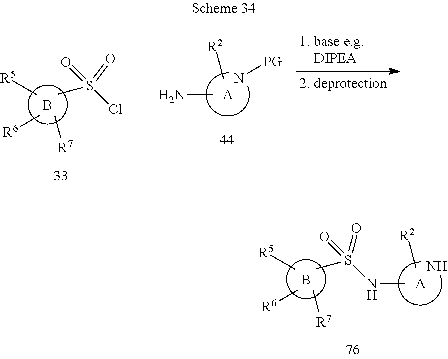

Alternatively, building blocks of formula 77 with L₁ and L₂=bond and B is (hetero)aryl and C is a N-linked cyclic amine, can be prepared by reacting a suitably protected (spiro)cyclic amine (Y=I or Br) in two sequential cross coupling reactions, followed by deprotection. Most typically this involves a Ni-catalyzed cross-coupling between 10 (Y=I) and a building block 62 bearing both a bromide and boronic acid functionality to give bromo (hetero)aryl intermediate 63. Intermediate 63 could be coupled with an amine 78 (e.g. under Buchwald conditions, Pd-catalysis) followed by deprotection to give 77. (Scheme 35)

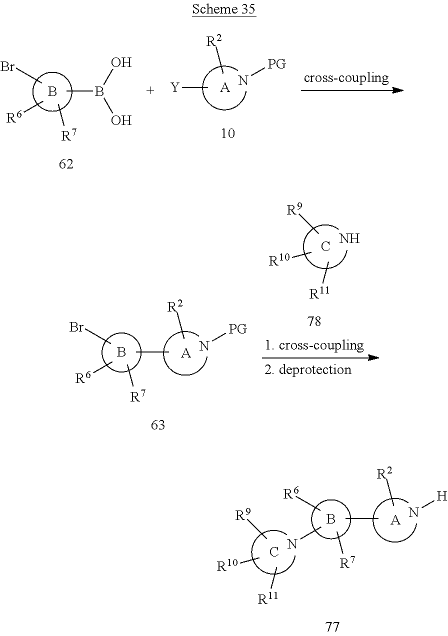

Building blocks with L₁=bond, L₂=O, and B=C-linked (hetero)aryl and C=(hetero)aryl (79) can be generated by SNAr reaction of a (hetero)aryl alcohol 80 with a dihalo(hetero)aryl 47 (X₁, X₂=halide e.g. Br) (e.g. using K₂CO₃), followed by a cross-coupling to install the A ring (e.g. photochemical cross-coupling using Ir[dF(CF₃)ppy]₂(dtbbpy)PF₆, NiCl₂·DME, dtbbpy and (TMS)₃SiH), followed by deprotection. (Scheme 36)

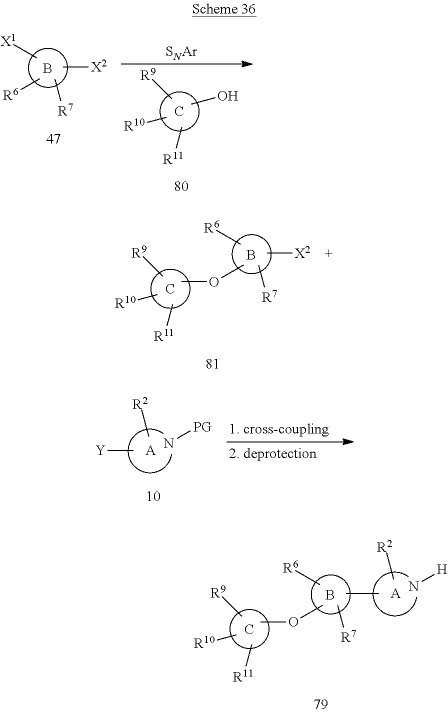

Alternatively, building blocks with L₁=bond, L₂=O, and B=C-linked (hetero)aryl or C₃-C₁₀-cycloalkyl and C=(hetero)aryl (79) can be generated by a cross-coupling between (spiro)cyclic amine 10 (Y=Br, I) and halophenol building block 82 (X₁=Br, I) (e.g. photochemical cross-coupling using Ir[dF(CF₃)ppy]₂(dtbbpy)PF₆, NiCl₂·DME, dtbbpy and (TMS)₃SiH), followed by SNAr reaction of phenol 83 with (hetero)aryl halide 84 (X₂=F, Cl, Br) (e.g. using K₂CO₃), and deprotection. (Scheme 37) A similar scheme is also suitable for installing small (cyclic)aliphatic units onto the phenol oxygen via S_{N}2 reaction. In some instances, in particular when B=C₃-C₁₀-cycloalkyl, intermediates of general formula 83 is commercially available and can be used directly in the aromatic substitution step.

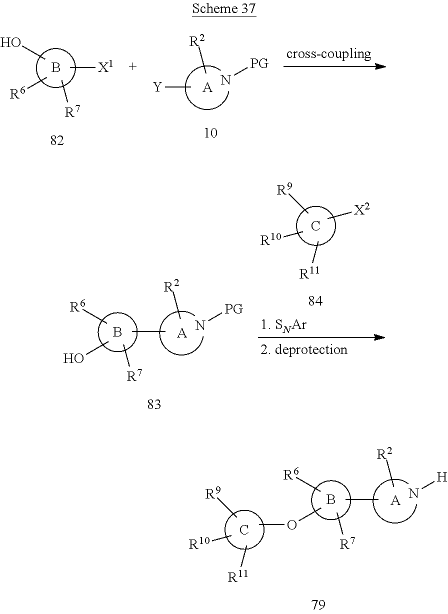

Building blocks of formula 61, with L₁ and L₂=bond, B=(hetero)aryl and C=C₃-C₁₀-cycloalkyl, 3- to 14-membered heterocyclyl, can be prepared by reacting a suitably protected (spiro)cyclic amine 10 (Y=I or Br) in two sequential cross coupling reactions, followed by deprotection. Most typically this involves a Negishi cross-coupling with a zincate transiently generated from 10 (Y=I) and a building block 85 bearing two bromide functionalities, to give bromo (hetero)aryl intermediate 63. Cross-coupling between intermediate 63 and bromide 86 in a photochemical reaction using Ir[dF(CF₃)ppy]₂(dtbbpy)PF₆, NiCl₂·DME, dtbbpy and (TMS)₃SiH followed by deprotection generates building blocks of general formula 61. (Scheme 38)

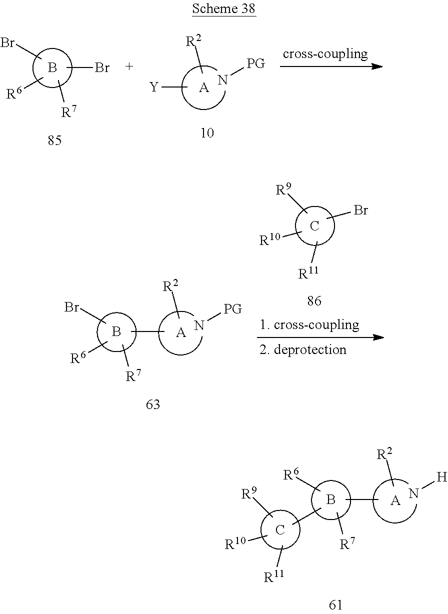

Building blocks of formula 3 where R³=H and R⁴=halo-C₁-C₆-(cyclo)alkyl, can be generated by cyclization between nitrile 87 and carbohydrazide 88 in the presence of a base such as K₂CO₃, in a polar protic solvent such as n-BuOH, to give intermediate 89. Building block 3 can be generated via deprotection of intermediate 89 (e.g. using TsOH or TFA where PG=Boc, or hydrogenation with Pd(OH)₂/C catalyst where PG=Cbz). (Scheme 39) If, due to the required protecting group, the cyano building block 87 was non commercial, this could be conveniently generated from the alcohol via conversion to the mesylate (e.g. using MsCl, Et₃N) and S_{N}2 displacement with a cyano group (e.g. using NaCN in DMF).

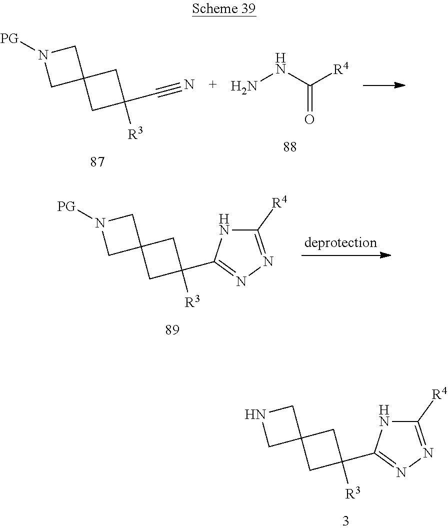

Alternatively, where R⁴=H, building blocks of formula 3 could be generated by heating a suitably protected amide in the presence of DMF-DMA, followed by heating in the presence of hydrazine and acetic acid to generate the 1,2,4-triazole, and deprotection under standard conditions (e.g. using TsOH or TFA where PG=Boc).

Alternatively, when building blocks of formula 3 where R³=H and R⁴=group —C(R^{4a}R^{4b}R^{4c}) where R is halo-C₁-C₆-(cyclo)alkyl; R^{4b} is hydroxy; and R^{4c} is hydrogen, the nitrile building block 87 can be converted to the corresponding thioamide 90 using for example using (NH₄)₂S and MgCl₂. Intermediate 90 can in turn be converted to carboxyimidothioate 91 using Mel in a solvent such as acetone or THF. Triazole 89 can be obtained after cyclization between intermediates 88 and 91 in the presence of a base such as K₂CO₃, in a polar protic solvent such as n-BuOH. Building block 3 can be generated from intermediate 89 after a deprotection step (e.g. using TsOH or TFA where PG=Boc). (Scheme 40)

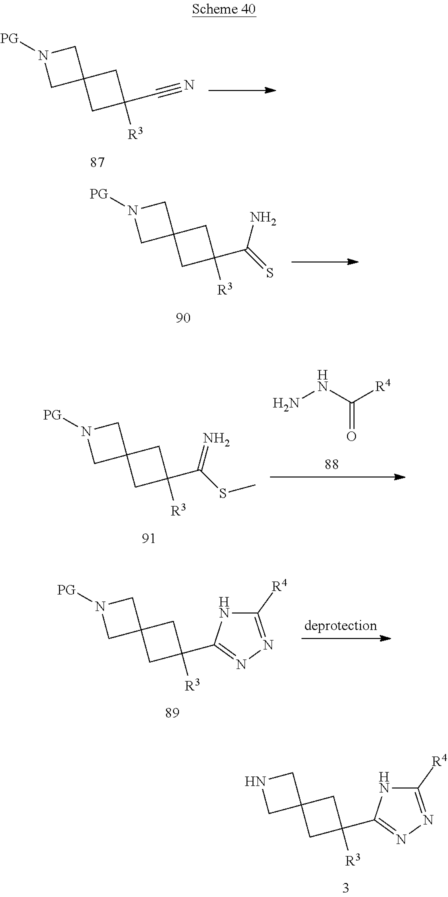

Alternatively compounds of formula I, where the A-ring is C-linked to the carbonyl can be generated by the amide coupling of acid 92 with amine 3 under standard conditions, e.g. using HATU and DIPEA in DMF. (Scheme 41)

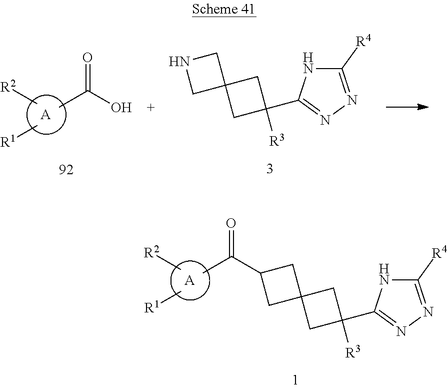

Alternatively, acid building blocks of general formula 93 with L₁=NH, A is C-linked, and B is (hetero)aryl can be prepared using SNAr reaction (e.g. using DIPEA, DMF) between (hetero)arylhalide 39 (X=F, Cl, Br, adjacent to pyridyl-type nitrogen) and amine 94, followed by deprotection of the methyl ester to the carboxylic acid (e.g. using aqueous base such as LiOH (aq.)). (Scheme 42)

It can also be understood that in Schemes 3-38, 43-45, and 47-61, the N-PG on the A ring can also be a suitably protected carboxylic acid equivalent (e.g. CH(COOPG)), and the same synthetic sequences can be carried out to further functionalize the A ring, to generate additional acid building blocks 92.

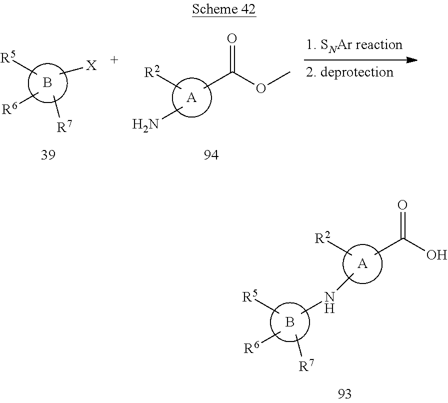

Building blocks of formula 95 where L₁=bond and L₂=-NHCH₂— can be generated by Curtius rearrangement of suitably protected acid building block 96 (e.g. using diphenylphosphonic azide, benzyl alcohol) and a deprotection of the generated carbamate (e.g. with Pd/C, H₂) to give amine 97, followed by further reductive amination of amine 97 with a suitable aldehyde 98, and subsequent deprotection. (Scheme 39) Alternatively the amine 97 can be used in a heterocylic synthesis reaction to generate an N-linked heterocylic C ring. (e.g. condensation reaction with a suitable 1,3-dione O-(4-nitrobenzoyl)hydroxylamine to generate a pyrazole).

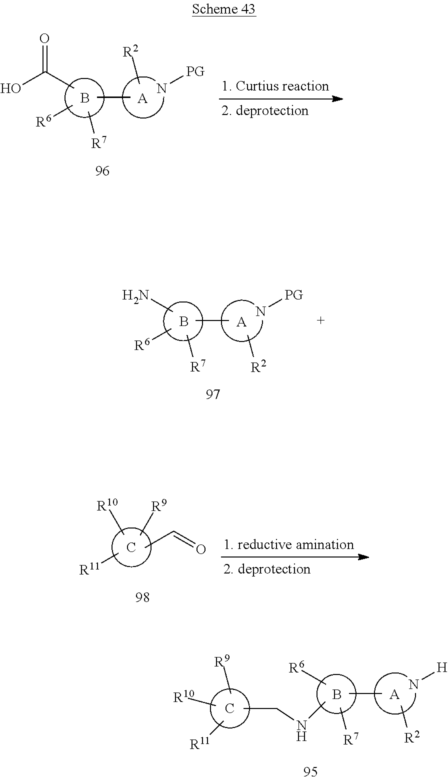

Alternatively, building blocks of general formula 99 where L₁=bond and L₂=CH₂, and B is a C-linked heteroaryl or a C3-C10-(cyclo)alkyl, can be prepared by a Mitsunobu type reaction of heterocycle B (100) with a hydroxyl building block 101 (e.g. using diisopropyl azodicarboxylate and triphenylphosphine, or Tsunoda reagent (cyanomethylenetrimethyiphosphorane)), followed by deprotection under standard conditions (e.g. with TsOH when PG=Boc). (Scheme 44) Alternatively, building blocks of formula 99 can be prepared by conversion of hydroxyl building block 101 to a mesylate (e.g. using MsCl, Et₃N) followed by an S_{N}2 reaction with the heterocycle B (100) in the presence of a base such as NaH.

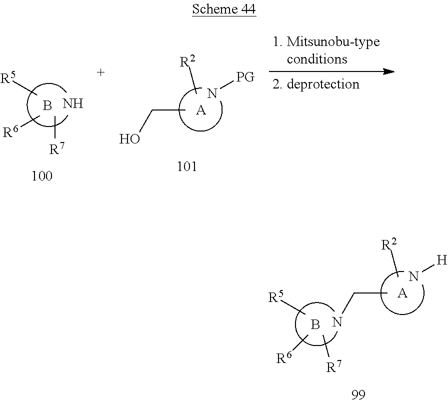

Alternatively, building blocks of formula 102 where B is a C-linked heteroaryl and L₁=-CH₂— may be generated using standard heterocyclic synthesis techniques starting from a suitable carboxylic acid or (104) or ester, nitrile (105) or diketone (106) derivative. The nitrite derivatives can be generated from the hydroxyl derivatives (101) via conversion to a mesylate (e.g. using MsCl. Et₃N) followed by S_{N}2 displacement of the mesylate group with cyanide (e.g. using KCN). The diketone derivatives can be generated from the commercial ester derivatives (103). (Scheme 45)

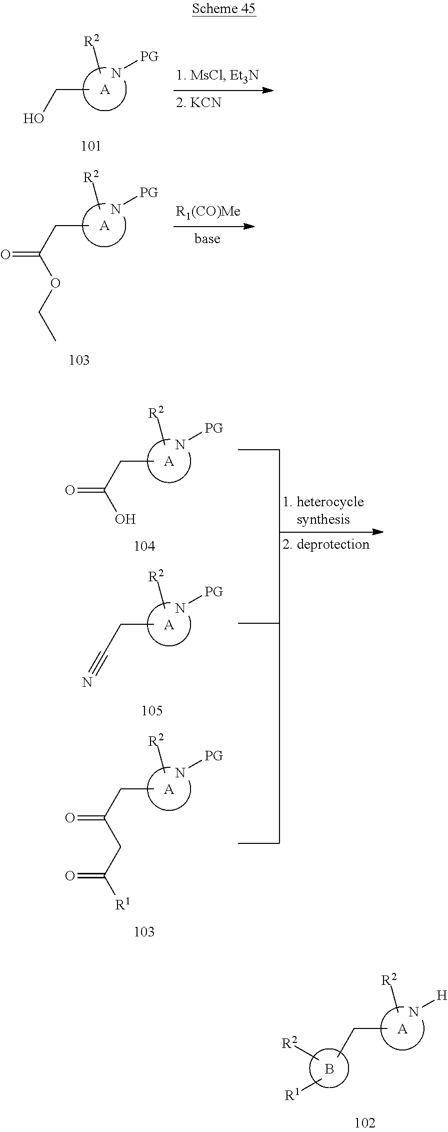

(Hetero)aryl trifluoromethylcyclopropyl building blocks 107 were not generally available, and were instead generated from halide building block 108 (X=I, Br) via Suzuki reaction with 1-(trifluoromethyl)vinylboronic acid to give 109. Cyclopropanation using diphenyl(methyl)sulfonium tetrafluoroborate and LiHMDS gave the required building block 107. (Scheme 46) This sequence could also be carried out in-between steps in other synthetic schemes, e.g. for N-linked heteroaryl rings B, these functionalization steps could also be carried out while making building blocks of formula 4, after the Suzuki/hydrogenation sequence, but prior to final deprotection (see Scheme 3). Alternatively the (hetero)aryl trifluoromethylcyclopropyl building blocks could be generated from commercial building blocks via standard heterocyclic synthesis techniques.

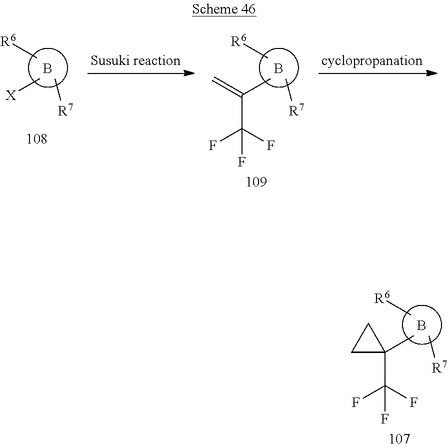

Building blocks of formula 61, where L₁ and L₂=bond, B=C₃-C₁₀-cycloalkyl and C=C₃-C₁₀-cycloalkyl, 3- to 14-membered heteroaryl, can be prepared by the reaction of a redox-active ester 110 with intermediate 86. A carboxylic acid 96 can be activated with a coupling agent such as DCC in the presence of DMAP and 2-hydroxyisoindoline-1,3-quinone to give the corresponding phthalimidooxycarbonyl 110. Photoreaction of intermediate 110 with bromide 86 in the presence of Ni(dtbbpy)Br₂, a base such as NaHCO₃, and 2,6-dimethyl-1,4-dihydropyridine-3,5-dicarboxylic acid diethyl ester under an LED lamp generated building blocks of general formula 61, after suitable deprotection. (Scheme 47)

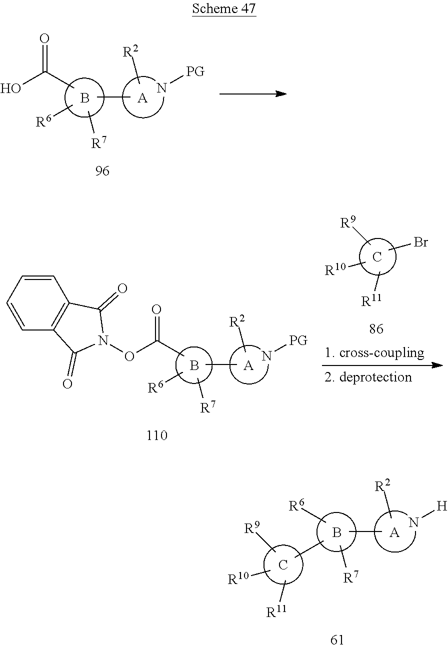

Building blocks of general formula 61, where L₁ and L₂=bond, B=C₃-C₁₀-cycloalkyl and C=3- to 14-membered heteroaryl, can be prepared by reacting a suitably protected, commercially available intermediate 111 with a Grignard reagent 112 (commercially available or generated in situ from the corresponding bromide or iodide) in an iron-catalyzed Kumada-type cross-coupling, using TMEDA and Fe(acac)₃ in a polar aprotic solvent such as THF, followed by deprotection. (Scheme 48)

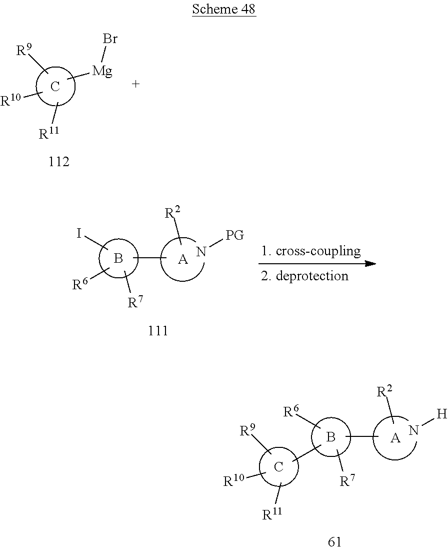

Building blocks of general formula 113, where L₁=bond, L₂=CH₂, B=C₃-C₁₀-cycloalkyl and C=3- to 14-membered heteroaryl, can be prepared starting from aldehyde building block 70, which, upon condensation with 4-methylbenzenesulfonhydrazide (114), gives tosylhydrazones 115. Such intermediates 115 can then be reacted with a commercially available, suitably protected boronic acid 116 (commercially available or prepared using standard chemistry from the corresponding halides) in the presence of a base such as cesium carbonate, to give, after a deprotection step, the corresponding intermediate of general formula 113. (Scheme 49)

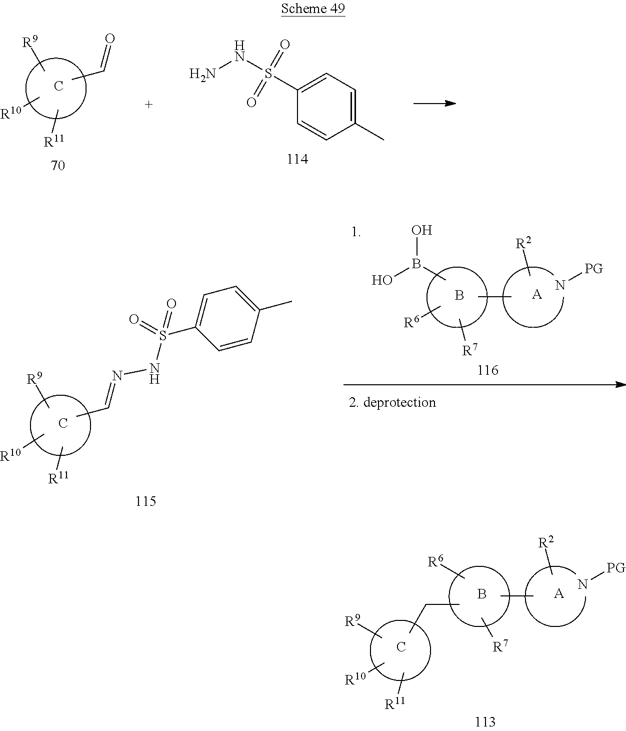

Building blocks of formula 117, where L₁=bond, L₂=CF₂, B=C₃-C₁₀-cycloalkyl and C=3- to 14-membered heteroaryl can be prepared starting from commercially available building block 96. This suitable protected acid 96 can be treated with CDI and N,O-dimethylhydroxylamine hydrochloride, to generate the corresponding Weinreb anode of general formula 118. Ketone intermediate 119 can be obtained by reacting Weinreb amide 118 with Grignard reagent 112 (which can be commercially available or prepared in situ from the corresponding halide following standard procedures). Intermediate of general formula 117 can be obtained via fluorination of ketone intermediate 119 using a fluorinating agent such as bis(2-methoxyethyl)aminosulfur trifluoride followed by a suitable deprotection. (Scheme 50)

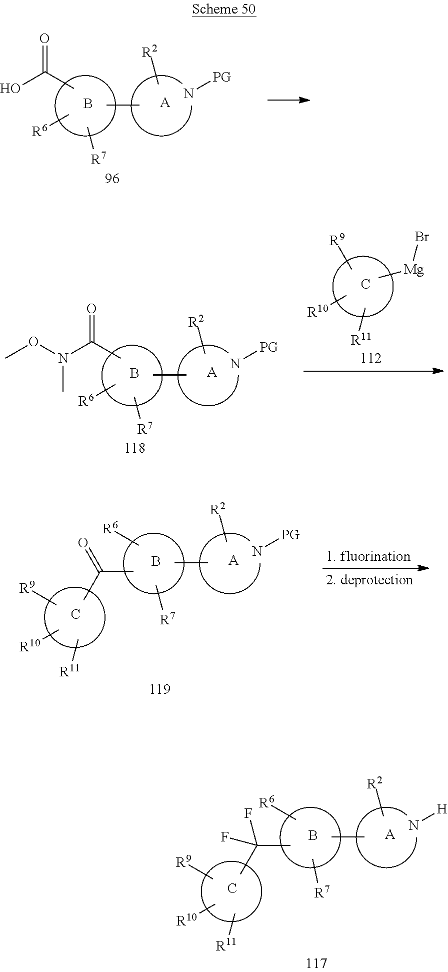

Alternatively, building blocks of general formula 113, where L₁=bond, L₂=CH₂, B=C₃-C₁₀-cycloalkyl and C=3- to 14-membered heteroaryl can be prepared starting from a suitably protected carboxylic acid 96. Acid 96 can be treated with isobutylchloroformate under basic conditions (e.g. 4-methylmorpholine) in a polar solvent such as THF, to give the corresponding intermediate 120, which can be transformed to the corresponding azide 121 using diazomethane (with caution). Intermediates of formula 121 give the corresponding Amst-Eistert-type product 122 upon treatment with silver trifluoroacetate in the presence of a base such as trimethylamine. Coupling of intermediate 122 with a carbohydrazide 123 and a coupling agent such as CDI gives intermediates 124. Building blocks of general formula 113 can be obtained following condensation of intermediate 124 using for example p-toluenesulfonyl chloride under basic conditions and suitable deprotection. (Scheme 51)

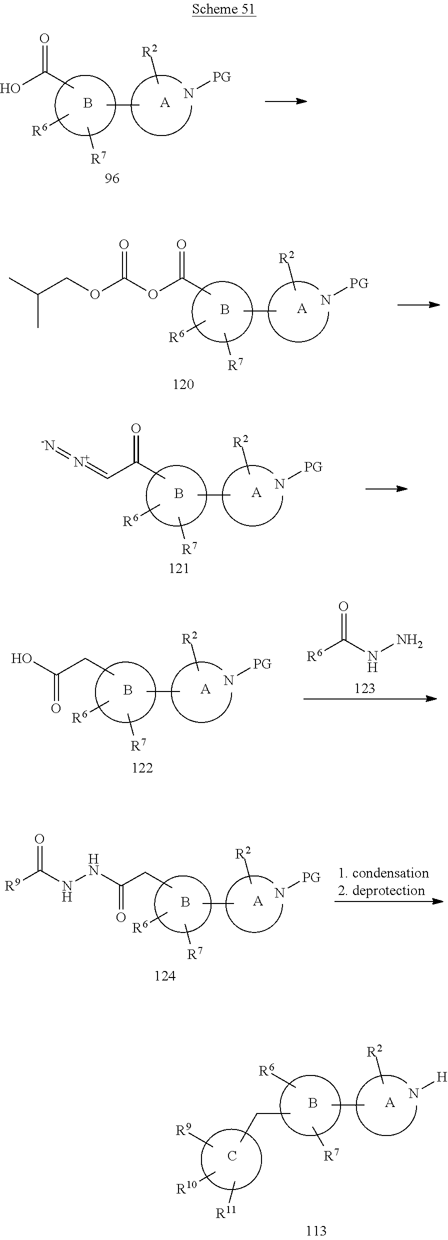

Alternatively, building blocks of general formula 61 where L₁=bond, L₂=bond, B=C₃-C₁₀-cycloalkyl and C=3- to 14-membered heteroaryl can be generated from suitably protected carboxylic acid %. Diketone 125 can be obtained from the reaction of acid 96 with a ketone 126 in the presence of 4-methylmorpholine, isobutyl chloroformate, and diisopropylamine. Following standard heterocycle synthesis conditions and a suitable deprotection step, building blocks of general formula 61 can be obtained. (Scheme 52)

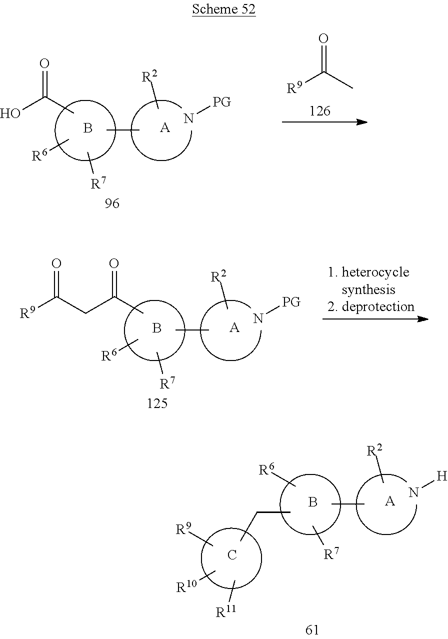

Building blocks of general formula 127, where L₁=bond, L₂=OCH₂, B=C₃-C₁₀-cycloalkyl and C=3- to 14-membered heteroaryl can be prepared starting from suitable protected carboxylic acids 96. Reduction of acid 96 with a reducing agent such as borane methyl sulfide complex gives the corresponding alcohol 128. Alcohol 128 can be transformed to the corresponding mesylate 129 using MsCl in the presence of a base. Intermediate of general formula 127 can be obtained from the reaction between alcohols of formula 80 and mesylate 129 under basic conditions (e.g. NaH), followed by a suitable deprotection step. (Scheme 53)

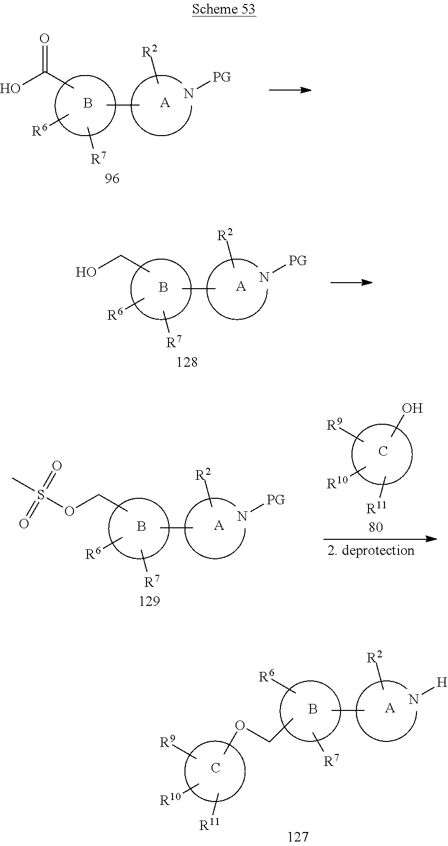

Alternatively, building blocks of general formula 130, where L₁=bond, L₂=NH, B=C₃-C₁₀-cycloalkyl and C=3- to 14-membered heteroaryl can be prepared starting from a Curtius rearrangement of a suitably protected acid building block 96 (e.g. using diphenylphosphonic azide, benzyl alcohol) and a deprotection of the generated carbamate (e.g. with Pd/C, H₂), to give amine 97. SNAr between amine 97 and (hetero)aryl halide 84 (X²=F, Cl, Br) in the presence of a base (e.g. trimethylamine) generates intermediates of general formula 130. (Scheme 54)

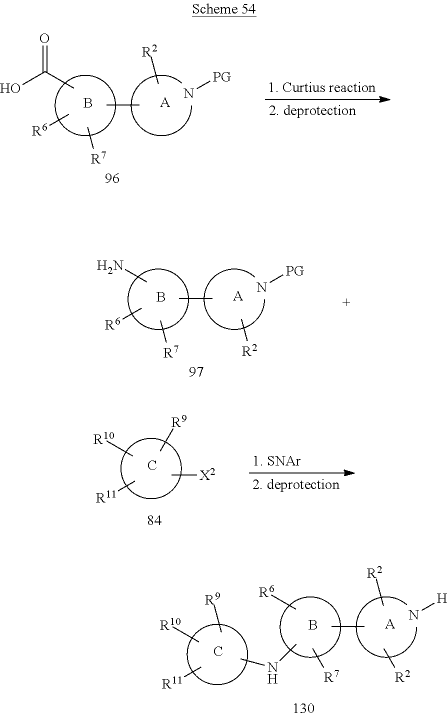

Alternatively, building blocks of general formula 131, where L₁=bond, L₂=SO₂, B=C₃-C₁₀-cycloalkyl and C=3- to 14-membered heteroaryl can be prepared starting from the iodine-promoted dimerization of thiol building block 132 to give disulfide intermediate 133. A suitably protected carboxylic acid 96 can be transformed to the corresponding Barton ester in situ, which following treatment with 133 and irradiation with a 500 W halogen lamp gives intermediates of formula 134. Building blocks of general formula 131 can be obtained following oxidation of the thioether 134 with oxidants such as mCPBA and suitable deprotection of the protecting group. (Scheme 55)

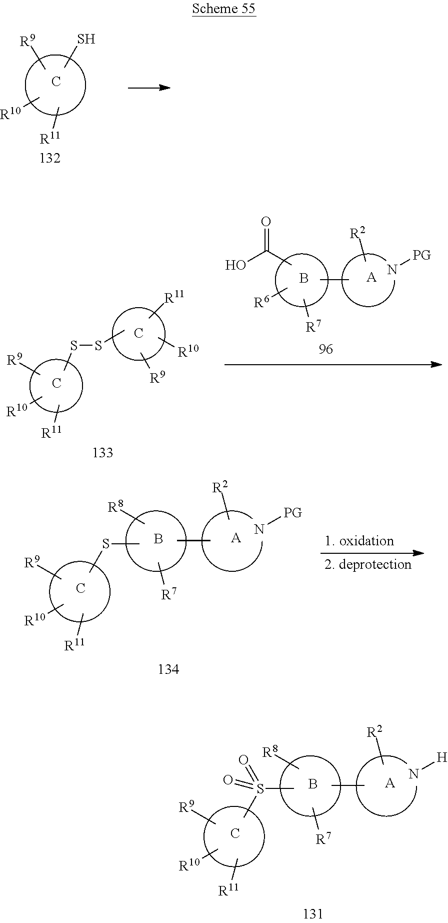

Alternatively, building blocks of general formula 131, where L₁=bond, L₂=SO₂, B=C₃-C₁₀-cycloalkyl and C=C₃-C ₁₀-cycloalkyl can be prepared starting from a suitably protected intermediate 135 (generated as described in Scheme 55). Intermediate 136 can be obtained upon treatment of 135 with a base such as NaH and ethanodiol. Alkylation of intermediate 136 with an alkyl halide 137 (X=Cl, Br, I) followed by deprotection can generate intermediates of general formula 131. (Scheme 56)

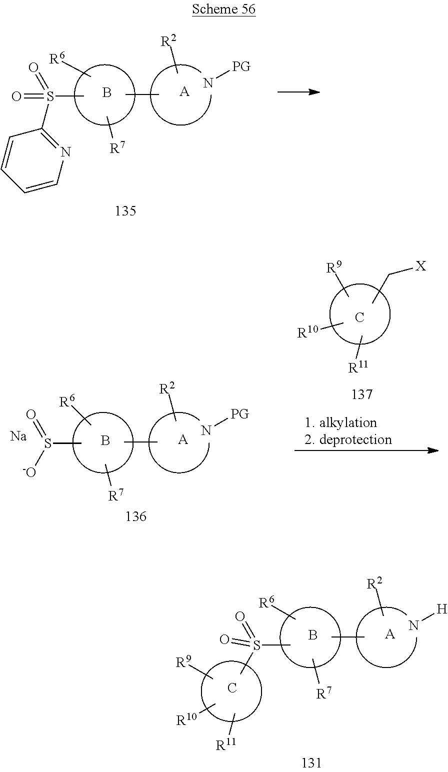

Alternatively, building blocks of general formula 57 with L₁=bond and B=C-linked (hetero)aryl or C₃-C₁₀-cycloalkyl and C=N-linked heteroaryl can be generated by Chan-Lam coupling (in presence of Cu(OAc)₂) of a suitably protected boronic acid 116 with the intermediate of general formula 54 to give, after a deprotection step, intermediate 57. (Scheme 57)

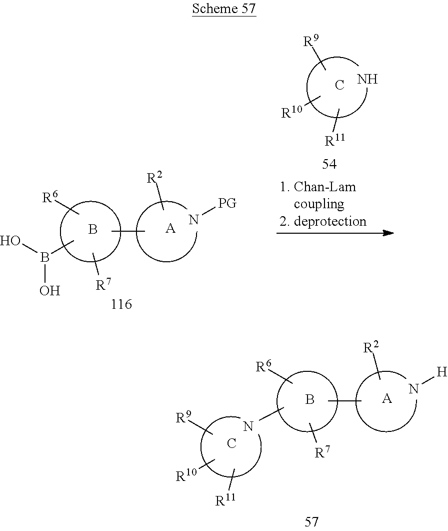

Alternatively, building blocks of general formula 8 with L₁=bond, B=C-linked (hetero)aryl and R⁵=hydroxy-C₁-C₆-alkyl can be generated by coupling a suitably protected Weinreb amide 138 with an aryl halide 139 (Y=₁ or Br) in the presence of n-BuLi, to generate a ketone intermediate 140. Standard heterocycle synthesis followed by deprotection yields building blocks of type 8. For example, an indazole B ring can be generated by using hydrazine under basic conditions. Further modifications on intermediate 8 may also be carried out prior to deprotection. (Scheme 58)

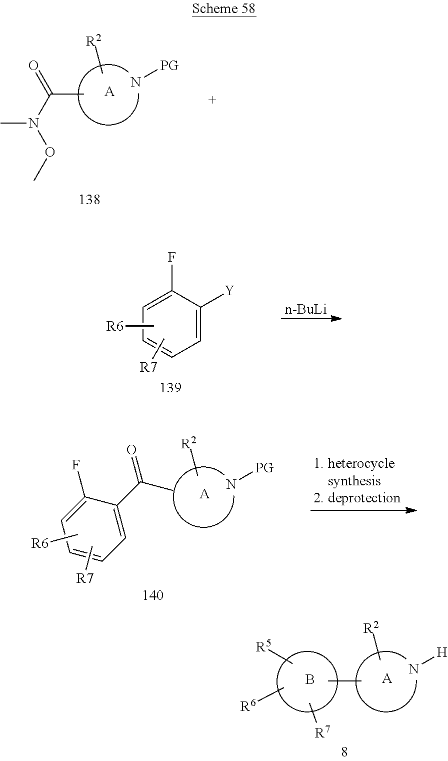

Alternatively, building blocks of formula 141 with L₁=C═O and A is N-linked can be prepared from an acyl chloride 142 and suitably protected amine A (30) in the presence of a base such as DIPEA or TEA, followed by deprotection under standard conditions (e.g. with TsOH, TFA or HCl when PG=Boc). (Scheme 59)

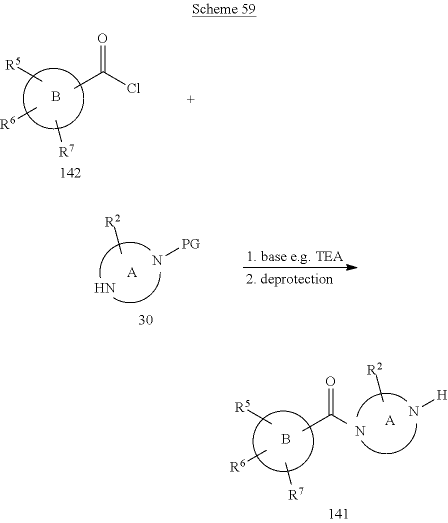

Building blocks of formula 143, where L₁=CF₂, can be generated via deoxyfluorination of a suitable ketone 145 (e.g. using DAST, diethylaminosulfur trifluoride), followed by a suitable deprotection (e.g. using TsOH or TFA, where PG=Boc). The ketone can be generated using oxidation of a benzylic CH₂ group on a suitable intermediate 144 (generated in Scheme 3), e.g. using SeO₂, or alternatively via nucleophilic attack of a metallated-anion derivative of a suitable (hetero)aryl 147 onto a Weinreb amide 148. The intermediate 147 can be generated from a suitable halogenated (hetero)aryl derivate 146 (X=Br, I, Cl) via halogen-metal exchange (e.g. using nBuLi, M=Li). (Scheme 60)

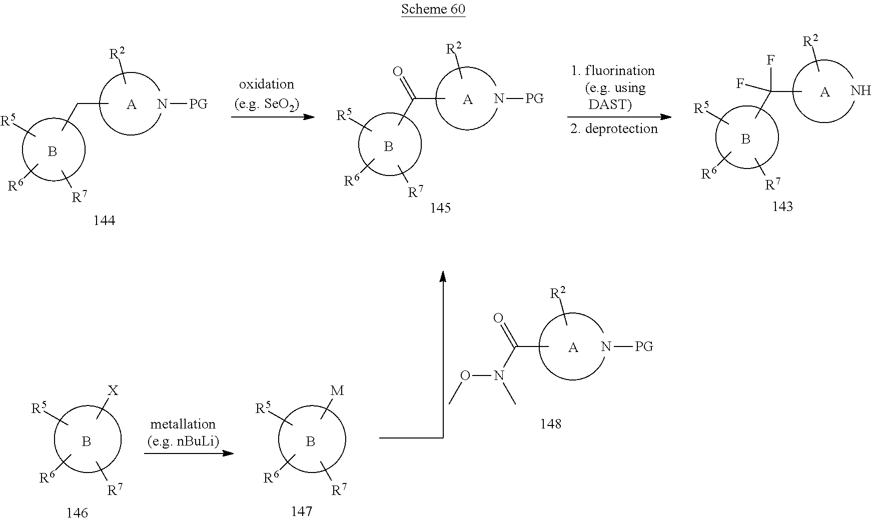

Building blocks of formula 149, where L₁=CFH, can be generated via deoxyfluorination of a suitable alcohol 151 (e.g. using 4-Morpholinylsulfur trifluoride or diethylaminosulfur trifluoride (DAST)), followed by a suitable deprotection (e.g. using TsOH or TFA, where PG=Boc). The alcohol 151 can be generated via nucleophilic attack of a metallated-anion derivative of a suitable (hetero)aryl 147 (wherein M is, for example, Li) onto a aldehyde 150. If required, the aldehyde 150 can be generated from an alcohol (via oxidation, e.g. using Dess-Martin periodinane, DMP) or from an acid (via reduction, e.g. using borane). (Scheme 61)

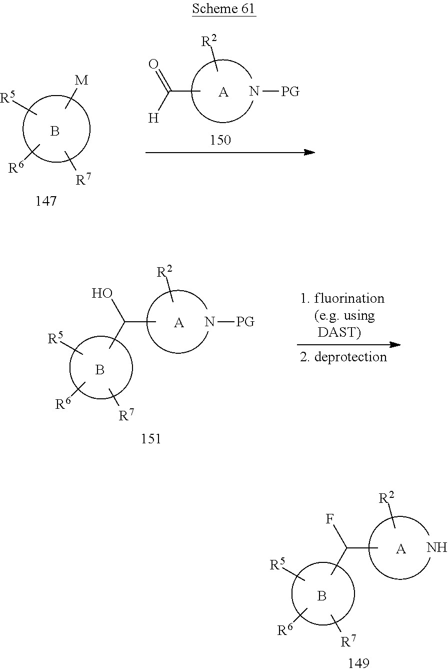

Building blocks of formula 1 where L₁=—CH₂CH₂— can be generated via a Wittig-type coupling between the A and B rings to generate an alkene, followed by reduction.

Building blocks of formula 1 where B is a cyclic amine, C=(hetero)aryl, L₁=bond, and L₂=-CH₂— N-linked to B-ring can be generated via reductive amination in a similar process as to that depicted above.

Building blocks of formula I where L₁=—CHR⁸—, B is (hetero)aryl and R⁸ is carbamoyl can be generated by aldol condensation of a (hetero)aryl acetonitrile and a suitably protected ketone building block 26 (using e.g. sodium ethanolate), followed by reduction of the double bond (e.g. Pd/C, H₂), conversion of the nitrite to the carbonyl group and deprotection.

In some cases, the R⁴ group contained an —NH₂ group. In these cases, typically the amine would be carried through the synthesis with Boc protection, and final deprotection to yield the —NH₂ group (e.g. using TFA) after the urea coupling step. In these cases the synthesis of the amine building block 3 would be carried out with an orthogonal protecting group, (e.g. Cbz), which could be deprotected via hydrogenation. (see Scheme 39 and 40, PG=Cbz)

In some cases, compounds of formula I could also be generated by combining the steps already described in new combinations e.g. carrying out the coupling in Scheme 1 prior to elaboration of the individual building blocks using the same sequences as described above.

In some cases, compounds of formula I could be further functionalized to give other compounds of formula I. For example, a compound of formula I bearing a (hetero)aryl bromide or iodide can be further functionalized with other groups e.g. small amine, small alkyl using metal catalyzed cross-coupling conditions such as Buchwald or Suzuki reactions. Building blocks 1 can also be subjected to further functionalization reactions (e.g. formation of an amide under standard conditions, alkylation of an alcohol (e.g. using NaH and an alkylating agent in DMF), conversion of boron-containing groups to hydroxyl using alkaline peroxide conditions, oxidation of thioethers to sulfones, or installation of small alkyl groups in place of Br or I groups using metal catalyzed cross-coupling conditions such as Buchwald or Suzuki reactions) before or after deprotection of the nucleophilic amine, to yield other building blocks of formula 1. A compound of formula I bearing a (hetero)aryl fluoride can be further functionalized via nucleophilic aromatic substitution, to introduce e.g. amines, ethers or thioethers.

In some cases, building blocks could be generated from commercially available fragments using standard functional group interconversion techniques (e.g. installation of a halide (e.g. using NIS or NBS, removal of a halide (e.g. under hydrogenation conditions), conversion of halides to other groups e.g. small amine, small alkyl using metal catalyzed cross-coupling conditions such as Buchwald or Suzuki reactions, installation and removal of protecting groups, hydrolysis of an ester to an acid, generation of an amide from an acid and a small amine, conversion of boron-containing groups to hydroxyl using alkaline peroxide conditions, cycloaddition of azidotrimethylsilane with a nitrite to generate a tetrazole, Sandmeyer reaction of an aniline to a bromide, oxidation of thioethers to sulfones, oxidation of thioethers to sulfoximines using PhI(OAc)₂ and NH₂COONH₄, alkylation of hydroxyl or amine groups via S_{N}2 reaction or reductive amination, acylation using an activated carbonyl derivative, or installation of —SO₂Me or —SO₂CF₃ groups from a iodo- or bromo-building block using literature techniques, installation of small alkyl groups or rings such as cyclopropyl onto heteroaromatic nitrogen using either S_{N}2 alkylation or Chan-Lam type conditions using an alkylboronate, cyclopropanation of an alkene, or installation of a dimethylphosphoryl group in place of a (hetero)aromatic halide (X=Br, I) group via Pd-catalyzed cross-coupling with dimethylphosphine oxide in the presence of a base such as Et₃N). Such techniques may also be used to elaborate commercially available fragments before, after, or intermediate within the synthetic sequences described above.

In one aspect, the present invention provides a process of manufacturing a compound of formula (I) described herein, or a pharmaceutically acceptable salt thereof, wherein the process is as described in any one of the schemes above.

In one aspect, the present invention provides a process for manufacturing a compound of formula (I) described herein, or a pharmaceutically acceptable salt thereof, comprising reacting a compound of formula 2;

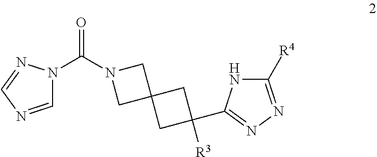

wherein R³ and R⁴ are as defined herein, with a compound of formula 1;

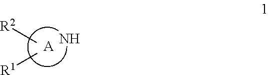

wherein R³ and R⁴ are as defined herein; by heating in a solvent, such as DMF or CH₃CN, in the presence of a base, such as DIPEA, to form said compound of formula (I).

In one aspect, the present invention provides a compound of formula (I) as described herein, or a pharmaceutically acceptable salt thereof, when manufactured according to any one of the processes described herein.

### MAGL Inhibitory Activity

Compounds of the present invention are MAGL inhibitors. Thus, in one aspect, the present invention provides the use of compounds of formula (I) as described herein for inhibiting MAGL in a mammal.

In a further aspect, the present invention provides compounds of formula (I) as described herein for use in a method of inhibiting MAGL in a mammal.

In a further aspect, the present invention provides the use of compounds of formula (I) as described herein for the preparation of a medicament for inhibiting MAGL in a mammal.

In a further aspect, the present invention provides a method for inhibiting MAGL in a mammal, which method comprises administering an effective amount of a compound of formula (I) as described herein to the mammal.

Compounds of formula (I) were profiled for MAGL inhibitory activity by determining the enzymatic activity by following the hydrolysis of the natural substrate 2-arachidonoylglycerol (2-AG) resulting in arachidonic acid, which can be followed by mass spectrometry. This assay is hereinafter abbreviated “2-AG assay”. The 2-AG assay was carried out in 384 well polypropylene assay plates. Compound dilutions were made in 100% DMSO in a polypropylene plate in 3-fold dilution steps to give a final concentration range in the assay from 12.5 μM to 0.8 μM. Compound dilutions were added to MAGL protein in assay buffer (50 mM TRIS, 1 mM EDTA, 0.01% (v/v) Tween-20, 2.5% (v/v) DMSO). After shaking, the plate was incubated for 15 min at RT. To start the reaction, 2-arachidonoylglycerol in assay buffer was added. The final concentrations in the assay was 50 μM for MAGL protein and 8 μM 2-arachidonoylglyerol. After shaking and 30 min incubation at RT, the reaction was quenched by the addition of two assay volumes of acetonitrile containing 4 μM of d8-arachidonic acid. The amount of arachidonic acid formed was traced by an online SPE system (Agilent Rapidfire) coupled to a triple quadrupole mass spectrometer. A C18 SPE cartridge (Agilent G9205A) was used in an acetonitrile/water liquid setup. The mass spectrometer was operated in negative electrospray mode following the mass transitions 303.1→259.1 for arachidonic acid and 311.1→267.0 for d8-arachidonic acid. The activity of the compounds was calculated based on the ratio of intensities [arachidonic acid/d8-arachidonic acid].

TABLE 1

| | IC₅₀ MAGL |
|---|---|
| Ex. | [μM] |
| 1 | 0.00094 |
| 2 | 0.00019 |
| 3 | 0.00069 |
| 4 | 0.00489 |
| 5 | 0.00016 |
| 6 | 0.00115 |
| 7 | 0.00365 |
| 8 | 0.00013 |
| 9 | 0.00057 |
| 10 | 0.00005 |
| 11 | 0.00030 |
| 12 | 0.00081 |
| 13 | 0.00472 |
| 14 | 0.01480 |
| 15 | 0.00133 |
| 16 | 0.00151 |
| 17 | 0.01521 |
| 18 | 0.00030 |
| 19 | 0.00481 |
| 20 | 0.00074 |
| 21 | 0.00533 |
| 22 | 0.00080 |
| 23 | 0.00443 |
| 24 | 0.00070 |
| 25 | 0.00448 |
| 26 | 0.00002 |
| 27 | 0.00011 |
| 28 | 0.00094 |
| 29 | 0.00020 |
| 30 | 0.00113 |
| 31 | 0.00000 |
| 32 | 0.00005 |
| 33 | 0.00001 |
| 34 | 0.00001 |
| 35 | 0.00007 |
| 36 | 0.00005 |
| 37 | 0.00001 |
| 38 | 0.00001 |
| 39 | 0.00015 |
| 40 | 0.00040 |
| 41 | 0.00024 |
| 42 | 0.00088 |
| 43 | 0.00058 |
| 44 | 0.00224 |
| 45 | 0.00237 |
| 46 | 0.00090 |
| 47 | 0.00027 |
| 48 | 0.00013 |
| 49 | 0.00081 |
| 50 | 0.00019 |
| 51 | 0.00177 |
| 52 | 0.01872 |
| 53 | 0.17933 |
| 54 | 0.00280 |
| 55 | 0.02196 |
| 56 | 0.00074 |
| 57 | 0.02569 |
| 58 | 0.00032 |
| 59 | 0.00053 |
| 60 | 0.00403 |
| 61 | 0.00066 |
| 62 | 0.00806 |
| 63 | 0.00359 |
| 64 | 0.03791 |
| 65 | 0.00146 |
| 66 | 0.00191 |
| 67 | 0.00586 |
| 68 | 0.00536 |
| 69 | 0.02128 |
| 70 | 0.00725 |
| 71 | 0.00174 |
| 72 | 0.15732 |
| 73 | 0.00324 |
| 74 | 0.00077 |
| 75 | 0.00555 |
| 76 | 0.03798 |
| 77 | 0.00069 |
| 78 | 0.00392 |
| 79 | 0.04342 |
| 80 | 0.05832 |
| 81 | 0.02823 |
| 82 | 0.02053 |
| 83 | 0.02574 |
| 84 | 0.03272 |
| 85 | 0.00102 |
| 86 | 0.00092 |
| 87 | 0.00821 |
| 88 | 0.04329 |
| 89 | 0.13105 |
| 90 | 0.00788 |
| 91 | 0.00405 |
| 92 | 0.04400 |
| 93 | 0.14278 |
| 94 | 0.00388 |
| 95 | 0.04751 |
| 96 | 0.00294 |
| 97 | 0.02795 |
| 98 | 0.00192 |
| 99 | 0.00497 |
| 100 | 0.06612 |
| 101 | 0.00370 |
| 102 | 0.01505 |
| 103 | 0.02504 |
| 104 | 0.19755 |
| 105 | 0.02127 |
| 106 | 0.02060 |
| 107 | 0.00314 |
| 108 | 0.03406 |
| 109 | 0.02012 |
| 110 | 0.00021 |
| 111 | 0.00060 |
| 112 | 0.00204 |
| 113 | 0.00009 |
| 114 | 0.00022 |
| 115 | 0.00020 |
| 116 | 0.00239 |
| 117 | 0.00010 |
| 118 | 0.00019 |
| 119 | 0.00652 |
| 120 | 0.04196 |
| 121 | 0.00001 |
| 122 | <0.00005 |
| 123 | 0.00398 |
| 124 | 0.00246 |
| 125 | 0.00008 |
| 126 | 0.00006 |
| 127 | 0.00006 |
| 128 | 0.00016 |
| 129 | 0.00010 |
| 130 | 0.00009 |
| 131 | 0.00279 |
| 132 | 0.00598 |
| 133 | 0.00173 |
| 134 | 0.00189 |
| 135 | 0.02532 |
| 136 | 0.00057 |
| 137 | 0.00679 |
| 138 | 0.00030 |
| 139 | 0.00387 |
| 140 | 0.00031 |
| 141 | 0.01056 |
| 142 | 0.00084 |
| 143 | 0.01448 |
| 144 | 0.00063 |
| 145 | 0.01263 |
| 146 | 0.00979 |
| 147 | 0.00029 |
| 148 | 0.00017 |
| 149 | 0.00039 |
| 150 | 0.00082 |
| 151 | 0.00051 |
| 152 | 0.00043 |
| 153 | 0.00027 |
| 154 | 0.00350 |
| 155 | 0.00199 |
| 156 | 0.00408 |
| 157 | 0.01580 |
| 158 | 0.15024 |
| 159 | 0.00608 |
| 160 | 0.06126 |
| 161 | 0.07882 |
| 162 | 0.03269 |
| 163 | 0.00411 |
| 164 | 0.01833 |
| 165 | 0.03087 |
| 166 | 0.07883 |
| 167 | 0.11289 |
| 168 | 0.03432 |
| 169 | 0.00062 |
| 170 | 0.02811 |
| 171 | 0.00921 |
| 172 | 0.50168 |
| 173 | 0.22968 |
| 174 | 0.04066 |
| 175 | 0.03429 |
| 176 | 0.00871 |
| 177 | 0.00038 |
| 178 | 0.00180 |
| 179 | 0.03361 |
| 180 | 0.00091 |
| 181 | 0.03617 |
| 182 | 0.00591 |
| 183 | 0.03132 |
| 184 | 0.01174 |
| 185 | 0.00165 |
| 186 | 0.04026 |
| 187 | 0.07055 |
| 188 | 0.02065 |
| 189 | 0.03863 |
| 190 | 0.01260 |
| 191 | 0.04564 |
| 192 | 0.02325 |
| 193 | 0.04555 |
| 194 | 0.01837 |
| 195 | 0.12993 |
| 196 | 0.11126 |
| 197 | 0.01558 |
| 198 | 0.17680 |
| 199 | 0.03193 |
| 200 | 0.06972 |
| 201 | 0.01699 |
| 202 | 0.33181 |
| 203 | 0.36305 |
| 204 | 1.35556 |
| 205 | 0.15498 |
| 206 | 0.23195 |
| 207 | 0.06028 |
| 208 | 0.021623 |
| 209 | 0.31578 |
| 210 | 0.48187 |
| 211 | 0.13620 |
| 212 | 0.05810 |
| 213 | 0.04503 |
| 214 | 0.01987 |
| 215 | 0.01743 |
| 216 | 0.01103 |
| 217 | 3.58154 |
| 218 | 0.17198 |
| 219 | 0.92256 |
| 220 | 0.20695 |
| 221 | 0.00851 |
| 222 | 0.04070 |
| 223 | 0.00033 |
| 224 | 0.00070 |
| 225 | 0.00367 |
| 226 | 0.00011 |
| 227 | 0.00091 |
| 228 | 0.01476 |
| 229 | 0.03394 |
| 230 | 0.00389 |
| 231 | 0.00339 |
| 232 | 0.04360 |
| 233 | 0.01378 |
| 234 | 0.13878 |
| 235 | 0.02189 |
| 236 | 0.00452 |
| 237 | 0.01709 |
| 238 | 0.35434 |
| 239 | 0.84155 |
| 240 | 0.00127 |
| 241 | 0.00024 |
| 242 | 0.00223 |
| 243 | 0.00057 |
| 244 | 0.00796 |
| 245 | 0.05017 |
| 246 | 0.06681 |
| 247 | 0.00370 |
| 248 | 0.67585 |
| 249 | 1.29810 |
| 250 | 0.04150 |
| 251 | 0.02390 |
| 252 | 0.06826 |
| 253 | 0.00014 |
| 254 | 0.00125 |
| 255 | 0.00068 |
| 256 | 0.00078 |
| 257 | 0.00008 |
| 258 | 0.00064 |
| 259 | 0.00363 |
| 260 | 0.01971 |
| 261 | 0.00036 |
| 262 | 0.00196 |
| 263 | 0.00821 |
| 264 | 0.00227 |
| 265 | 0.00438 |
| 266 | 0.00293 |
| 267 | 0.00031 |
| 268 | 0.00035 |
| 269 | 0.00229 |
| 270 | 0.00493 |
| 271 | 0.00273 |
| 272 | 0.00367 |
| 273 | 0.01069 |
| 274 | 0.00023 |
| 275 | 0.00207 |
| 276 | 0.00017 |
| 277 | 0.00017 |
| 278 | 0.00030 |
| 279 | 0.00516 |
| 280 | 0.00382 |
| 281 | 0.00102 |
| 282 | 0.00103 |
| 283 | 0.00088 |
| 284 | 0.00159 |
| 285 | 0.00037 |
| 286 | 0.00048 |
| 287 | 0.00013 |
| 288 | 0.00019 |
| 289 | 0.00045 |
| 290 | 0.02101 |
| 291 | 0.00057 |
| 292 | 0.01070 |
| 293 | 0.01115 |
| 294 | 0.00056 |
| 295 | 0.00141 |
| 296 | 4.11604 |
| 297 | 0.00316 |
| 298 | 0.00803 |
| 299 | 0.00005 |
| 300 | 0.00100 |
| 301 | 0.00169 |
| 302 | 0.00048 |
| 303 | 0.00631 |
| 304 | 0.00018 |
| 305 | 0.00007 |
| 306 | 0.00219 |
| 307 | 0.00044 |
| 308 | 0.00212 |
| 309 | 0.00220 |
| 310 | 0.00230 |
| 311 | 0.00235 |
| 312 | 0.00297 |
| 313 | 0.00459 |
| 314 | 0.00476 |
| 315 | 0.00493 |
| 316 | 0.00621 |
| 317 | 0.00718 |
| 318 | 0.00730 |
| 319 | 0.00804 |
| 320 | 0.00828 |
| 321 | 0.01080 |
| 322 | 0.01171 |
| 323 | 0.01235 |
| 324 | 0.01434 |
| 325 | 0.01470 |
| 326 | 0.02285 |
| 327 | 0.02488 |
| 328 | 0.03540 |
| 329 | 0.03813 |
| 330 | 0.05992 |
| 331 | 0.10943 |
| 332 | 0.15871 |
| 333 | 0.15893 |
| 334 | 0.27222 |
| 335 | 0.46731 |
| 336 | 0.05584 |
| 337 | 0.00007 |
| 338 | 0.00009 |
| 339 | 0.00016 |
| 340 | 0.00109 |
| 341 | 0.00012 |
| 342 | 0.00049 |
| 343 | 0.00097 |
| 344 | 0.00759 |
| 345 | 0.00915 |
| 346 | 0.00013 |
| 347 | 0.00031 |
| 348 | 0.00063 |
| 349 | 0.00248 |
| 350 | 0.00343 |
| 351 | 0.00359 |
| 352 | 0.00394 |
| 353 | 0.00512 |
| 354 | 0.00539 |
| 355 | 0.00886 |
| 356 | 0.01173 |
| 357 | 0.02564 |
| 358 | 0.02630 |
| 359 | 0.02844 |
| 360 | 0.03199 |
| 361 | 0.04563 |
| 362 | 0.10641 |
| 363 | 0.24448 |
| 364 | 0.37710 |
| 365 | 0.00006 |
| 366 | 0.00008 |
| 367 | 0.00015 |
| 368 | 0.00017 |
| 369 | 0.00018 |
| 370 | 0.00034 |
| 371 | 0.00040 |
| 372 | 0.00057 |
| 373 | 0.00058 |
| 374 | 0.00161 |
| 375 | 0.00189 |
| 376 | 0.00222 |
| 377 | 0.00429 |
| 378 | 0.01661 |
| 379 | 0.06098 |
| 380 | 0.06418 |
| 381 | 0.00069 |
| 382 | 0.02137 |
| 383 | 0.00209 |
| 384 | 0.00264 |
| 385 | 0.00376 |
| 386 | 0.24570 |
| 387 | 0.07962 |
| 388 | 0.41011 |
| 389 | 0.01521 |
| 390 | 0.00106 |
| 391 | 0.04826 |
| 392 | 0.00659 |
| 393 | 0.00120 |
| 394 | 0.00149 |
| 395 | 0.00128 |
| 396 | 0.00477 |
| 397 | 0.00242 |
| 398 | 0.00240 |
| 399 | 0.00263 |
| 400 | 0.00117 |
| 401 | 0.0004 |
| 402 | 0.00095 |
| 403 | 0.00075 |
| 404 | 0.00029 |
| 405 | 0.00057 |
| 406 | 0.00175 |
| 407 | 0.00924 |
| 408 | 0.00146 |
| 409 | 0.00482 |
| 410 | 0.00014 |
| 411 | 0.00029 |
| 412 | 0.04217 |
| 413 | 0.00034 |
| 414 | 0.0381 |
| 415 | 0.00443 |
| 416 | 0.01328 |
| 417 | 0.0062 |
| 418 | 0.19189 |
| 419 | 0.0005 |
| 420 | 0.00002 |
| 421 | 0.05045 |
| 422 | 0.01432 |
| 423 | 0.00111 |
| 424 | 0.00046 |
| 425 | 0.00222 |
| 426 | 0.27444 |
| 427 | 0.00133 |
| 428 | 0.00031 |
| 429 | 0.01183 |
| 430 | 0.00522 |
| 431 | 0.00039 |
| 432 | 0.00345 |
| 433 | 0.00090 |
| 434 | 0.00552 |
| 435 | 0.00395 |
| 436 | 0.03227 |
| 437 | 0.03957 |
| 438 | 0.00346 |
| 439 | 0.00422 |
| 440 | 0.10944 |
| 441 | 0.41378 |
| 442 | 0.73524 |
| 443 | 0.00247 |
| 444 | 0.00325 |
| 445 | 0.00024 |
| 446 | 0.00171 |
| 447 | 0.10126 |
| 448 | 0.00820 |
| 449 | 0.00045 |
| 450 | 0.00024 |
| 451 | 0.00152 |
| 452 | 0.41268 |
| 453 | 0.00173 |
| 454 | 0.12887 |
| 455 | 0.00710 |
| 456 | 0.00888 |
| 457 | 0.00031 |
| 458 | 0.02279 |
| 459 | 0.00002 |
| 460 | 0.00264 |
| 461 | 0.00100 |
| 462 | 0.00309 |
| 463 | 0.00056 |
| 464 | 0.00007 |
| 465 | 0.11387 |
| 466 | 0.02286 |
| 467 | 0.00033 |
| 468 | 0.00250 |
| 469 | 0.00112 |
| 470 | 0.00508 |
| 471 | 0.02026 |
| 472 | 0.01774 |
| 473 | 0.06785 |
| 474 | 0.00459 |
| 475 | 0.00008 |
| 476 | 0.00241 |
| 477 | 0.00008 |
| 478 | 0.00159 |
| 479 | 0.00018 |
| 480 | 0.01209 |
| 481 | 0.00106 |
| 482 | 0.00339 |
| 483 | 0.00213 |
| 484 | 0.08920 |
| 485 | 0.06451 |
| 486 | 0.01059 |
| 487 | 0.00982 |
| 488 | 0.11077 |
| 489 | 0.07529 |
| 490 | 0.00005 |
| 491 | 0.00034 |
| 492 | 0.00384 |
| 493 | 0.95577 |
| 494 | 0.00369 |
| 495 | 0.13980 |
| 496 | 0.03697 |
| 497 | 0.02227 |
| 498 | 0.19494 |
| 499 | 0.00346 |
| 500 | 0.00948 |
| 501 | 0.00092 |
| 502 | 0.26175 |
| 503 | 0.00069 |
| 504 | 0.00104 |
| 505 | 0.00130 |
| 506 | 0.00012 |
| 507 | 0.00584 |
| 508 | 0.00666 |
| 509 | 0.00516 |
| 510 | 0.02043 |
| 511 | 0.02083 |
| 512 | 0.00067 |
| 513 | 0.03186 |
| 514 | 0.00047 |
| 515 | n.a. |
| 516 | 0.00017 |
| 517 | 0.00015 |
| 518 | 0.00022 |
| 519 | 0.01392 |
| 520 | 0.00223 |
| 521 | 0.07862 |
| 522 | 0.00005 |
| 523 | 0.00053 |
| 524 | 0.00140 |
| 525 | 0.00708 |
| 526 | 0.00460 |
| 527 | 0.00098 |
| 528 | 0.49728 |
| 529 | 0.00031 |
| 530 | 0.01394 |
| 531 | 0.00753 |
| 532 | 0.00021 |
| 533 | 0.00557 |
| 534 | 0.00542 |
| 535 | 0.00065 |
| 536 | 0.00393 |
| 537 | 0.23732 |
| 538 | 0.00393 |
| 539 | 0.00925 |
| 540 | 0.00005 |
| 541 | 1.10980 |
| 542 | 0.08185 |
| 543 | 0.00002 |
| 544 | 0.00872 |
| 545 | 0.04690 |
| 546 | 0.13999 |
| 547 | 0.00586 |
| 548 | 0.00085 |
| 549 | 0.00066 |
| 550 | 0.00981 |
| 551 | 0.00018 |
| 552 | 0.02622 |
| 553 | 0.00455 |
| 554 | 0.00056 |
| 555 | 0.00034 |
| 556 | 0.00045 |
| 557 | 0.01241 |
| 558 | 0.00060 |
| 559 | 0.00046 |
| 560 | 0.00125 |
| 561 | 1.45428 |
| 562 | 0.00020 |
| 563 | 0.01273 |
| 564 | 0.00086 |
| 565 | 0.00104 |
| 566 | 0.01465 |
| 567 | 2.01026 |
| 568 | 0.40849 |
| 569 | 0.00009 |
| 570 | 0.03583 |
| 571 | 0.00055 |
| 572 | 0.18421 |
| 573 | 0.90405 |
| 574 | 0.06540 |
| 575 | 0.00402 |
| 576 | 0.08506 |
| 577 | 0.25053 |
| 578 | 0.00051 |
| 579 | 0.02396 |
| 580 | 0.67908 |
| 581 | 0.00222 |
| 582 | 0.16749 |
| 583 | 0.29510 |
| 584 | 0.00048 |
| 585 | 0.00006 |
| 586 | 0.00547 |
| 587 | 0.00240 |
| 588 | 0.04041 |
| 589 | 0.00059 |
| 590 | 0.00034 |
| 591 | 0.00364 |
| 592 | 0.00138 |
| 593 | 0.00136 |
| 594 | 0.00002 |
| 595 | 0.00363 |
| 596 | 0.35747 |
| 597 | 0.13537 |
| 598 | 0.00189 |
| 599 | 0.00084 |
| 600 | 0.00425 |
| 601 | 0.02639 |
| 602 | 0.00011 |
| 603 | 0.03441 |
| 604 | 0.00689 |
| 605 | 0.00086 |
| 606 | 0.00517 |
| 607 | 0.00008 |
| 608 | 0.00361 |
| 609 | 0.00832 |
| 610 | 0.00040 |
| 611 | 0.00068 |
| 612 | 0.00013 |
| 613 | 0.00091 |
| 614 | 0.00549 |
| 615 | 0.00165 |
| 616 | 0.00101 |
| 617 | 0.00816 |
| 618 | 0.09511 |
| 619 | 0.00079 |
| 620 | 0.18714 |
| 621 | 0.00362 |
| 622 | 0.00063 |
| 623 | 0.01263 |
| 624 | 0.00821 |
| 625 | 0.00875 |
| 626 | 0.05645 |
| 627 | 0.00005 |
| 628 | 0.14945 |
| 629 | 0.00145 |
| 630 | 0.00136 |
| 631 | 0.01072 |
| 632 | 0.01232 |
| 633 | 1.30680 |
| 634 | 0.10422 |
| 635 | 0.94980 |
| 636 | 0.00003 |
| 637 | 0.00744 |
| 638 | 0.00052 |
| 639 | 0.00021 |
| 640 | 0.04367 |
| 641 | 0.06744 |
| 642 | 0.02751 |
| 643 | 0.00667 |
| 644 | 0.00022 |
| 645 | 0.00654 |
| 646 | 0.42352 |
| 647 | 0.00097 |
| 648 | 1.50441 |
| 649 | 0.00012 |
| 650 | 0.00974 |
| 651 | 0.00948 |
| 652 | 0.00019 |
| 653 | 0.00100 |
| 654 | 0.00004 |
| 655 | 0.55728 |
| 656 | 0.03974 |
| 657 | 0.01723 |
| 658 | 0.41223 |
| 659 | 0.04091 |
| 660 | 0.00079 |
| 661 | 0.02194 |
| 662 | 0.01192 |
| 663 | 0.01306 |
| 664 | 0.00274 |
| 665 | 0.00023 |
| 666 | 0.00220 |
| 667 | 0.00009 |
| 668 | 0.00975 |
| 669 | 0.00065 |
| 670 | 0.00100 |
| 671 | 0.00555 |
| 672 | 0.00831 |
| 673 | 0.00200 |
| 674 | 0.00808 |
| 675 | 0.00876 |
| 676 | 0.00294 |
| 677 | 0.01297 |
| 678 | 0.03388 |
| 679 | 0.00015 |
| 680 | 0.00171 |
| 681 | 0.00741 |
| 682 | 0.03837 |
| 683 | 0.00021 |
| 684 | 0.00433 |
| 685 | 0.00096 |
| 686 | 0.01424 |
| 687 | 0.05412 |
| 688 | 0.00896 |
| 689 | 0.09676 |
| 690 | 0.00061 |
| 691 | 0.00016 |
| 692 | 0.02006 |
| 693 | 0.02454 |
| 694 | 0.23628 |
| 695 | 0.04378 |
| 696 | 0.04220 |
| 697 | 0.00265 |
| 698 | 0.05587 |
| 699 | 0.00034 |
| 700 | 0.00256 |
| 701 | 0.06439 |
| 702 | 0.25501 |
| 703 | 0.00263 |
| 704 | 0.00030 |
| 705 | 0.00057 |
| 706 | 0.28006 |
| 707 | 0.00244 |
| 708 | 0.00263 |
| 709 | 0.02426 |
| 710 | 0.00261 |
| 711 | 0.00020 |
| 712 | 0.49721 |
| 713 | 0.00342 |
| 714 | 0.03003 |
| 715 | 0.00045 |
| 716 | 0.02919 |
| 717 | 0.08378 |
| 718 | 0.54956 |
| 719 | 0.06893 |
| 720 | 0.00026 |
| 721 | 0.54401 |
| 722 | 0.05707 |
| 723 | 0.00137 |
| 724 | 0.00380 |
| 725 | 0.04657 |
| 726 | 0.00010 |
| 727 | 0.00268 |
| 728 | 0.00419 |
| 729 | 0.00009 |
| 730 | 0.00201 |
| 731 | 0.00018 |
| 732 | 0.00618 |
| 733 | 0.02953 |
| 734 | 0.56767 |
| 735 | 0.00234 |
| 736 | 0.00165 |
| 737 | 0.01203 |
| 738 | 0.32766 |
| 739 | 0.00126 |
| 740 | 0.00132 |
| 741 | 0.00015 |
| 742 | 0.00218 |
| 743 | 0.01316 |
| 744 | 0.01363 |
| 745 | 0.00637 |
| 746 | 0.00864 |
| 747 | 0.00009 |
| 748 | 0.03720 |
| 749 | 0.00025 |
| 750 | 0.44451 |
| 751 | 0.00066 |
| 752 | 0.00029 |
| 753 | 1.48771 |
| 754 | 0.00043 |
| 755 | 0.00061 |
| 756 | 0.09850 |
| 757 | 0.00377 |
| 758 | 0.14514 |
| 759 | 0.01103 |
| 760 | 0.00609 |
| 761 | 0.14702 |
| 762 | 0.03573 |
| 763 | 0.02709 |
| 764 | 0.00108 |
| 765 | 0.00449 |
| 766 | 0.00283 |
| 767 | 0.00407 |
| 768 | 0.00008 |
| 769 | 0.01177 |
| 770 | 0.00023 |
| 771 | 0.00702 |
| 772 | 0.00005 |
| 773 | 0.00131 |
| 774 | 0.00794 |
| 775 | 0.00201 |
| 776 | 0.00590 |
| 777 | 0.00127 |
| 778 | 0.00099 |
| 779 | 0.00836 |
| 780 | 0.00337 |
| 781 | 0.00093 |
| 782 | 0.01779 |
| 783 | 0.03367 |
| 784 | 0.00518 |
| 785 | 0.00039 |
| 786 | 0.00801 |
| 787 | 0.00120 |
| 788 | 0.02247 |
| 789 | 0.40408 |
| 790 | 0.00968 |
| 791 | 0.01763 |
| 792 | 0.00198 |
| 793 | 0.00007 |
| 794 | 0.00103 |
| 795 | 0.03335 |
| 796 | 0.00117 |
| 797 | 0.00931 |
| 798 | 0.00240 |
| 799 | 1.33220 |
| 800 | 0.55397 |
| 801 | 0.00816 |
| 802 | 0.00018 |
| 803 | 0.00139 |
| 804 | 0.00716 |
| 805 | 0.00473 |
| 806 | 0.00972 |
| 807 | 0.47846 |
| 808 | 0.00029 |
| 809 | 0.00034 |
| 810 | 0.00114 |
| 811 | 0.00043 |
| 812 | 0.00030 |
| 813 | 0.00018 |
| 814 | 0.06028 |
| 815 | 0.00060 |
| 816 | 0.00161 |
| 817 | 0.18026 |
| 818 | 0.00059 |
| 819 | 0.00659 |
| 820 | 0.00077 |
| 821 | 0.00200 |
| 822 | 0.00088 |
| 823 | 0.00021 |
| 824 | 0.01033 |
| 825 | 0.00437 |
| 826 | 0.00056 |
| 827 | 0.02119 |
| 828 | 0.42342 |
| 829 | 0.03844 |
| 830 | 0.03647 |
| 831 | 1.21706 |
| 832 | 0.00239 |
| 833 | 0.00039 |
| 834 | 0.00006 |
| 835 | 0.00237 |
| 836 | 0.00008 |
| 837 | 0.70708 |
| 838 | 0.10159 |
| 839 | 0.00027 |
| 840 | 0.00291 |
| 841 | 0.01064 |
| 842 | 0.13335 |
| 843 | 0.00107 |
| 844 | 0.00071 |
| 845 | 0.00983 |
| 846 | 0.00078 |
| 847 | 0.00053 |
| 848 | 0.00067 |
| 849 | 0.00007 |
| 850 | 0.01370 |
| 851 | 0.00023 |
| 852 | 0.00006 |
| 853 | 0.00001 |
| 854 | n.a. |
| 855 | n.a. |
| 856 | 0.00041 |
| 857 | 0.00002 |
| 858 | 0.00002 |
| 859 | 0.00019 |
| 860 | 0.00290 |
| 861 | 0.05963 |
| 862 | 0.00157 |
| 863 | 0.04425 |
| 864 | 0.01433 |
| 865 | 0.01937 |
| 866 | 0.01913 |
| 867 | 0.00010 |
| 868 | 0.00141 |
| 869 | 0.11526 |
| 870 | 0.82063 |
| 871 | 0.34151 |
| 872 | 1.04072 |
| 873 | 0.00067 |
| 874 | 0.00280 |
| 875 | 0.00033 |
| 876 | 0.80626 |
| 877 | 0.01521 |
| 878 | 0.01834 |
| 879 | 0.30034 |
| 880 | 0.83210 |
| 881 | 0.67020 |
| 882 | 0.00182 |
| 883 | 0.00039 |
| 884 | 0.00147 |
| 885 | 0.35177 |
| 886 | 0.00638 |
| 887 | 0.00598 |
| 888 | 0.00285 |
| 889 | 0.00274 |
| 890 | 0.00122 |
| 891 | 0.03398 |
| 892 | 0.01235 |
| 893 | 0.09516 |
| 894 | 0.07745 |
| 895 | 0.02353 |
| 896 | 0.20853 |
| 897 | 0.30840 |
| 898 | 0.21789 |
| 899 | 0.10025 |
| 900 | 0.02526 |
| 901 | 0.13574 |
| 902 | 3.11152 |
| 903 | 0.12542 |
| 904 | 0.15918 |
| 905 | 0.00592 |
| 906 | 0.00561 |
| 907 | 0.04313 |
| 908 | 0.05657 |
| 909 | 0.00051 |
| 910 | 0.00423 |
| 911 | 0.00003 |
| 912 | 0.00007 |
| 913 | 0.00044 |
| 914 | 0.00027 |
| 915 | 0.24121 |
| 916 | 0.03970 |
| 917 | 0.23725 |
| 918 | 0.96382 |
| 919 | 1.19930 |
| 920 | 0.00402 |
| 921 | 0.03145 |
| 922 | 0.32604 |
| 923 | 0.73068 |
| 924 | 0.04924 |
| 925 | 0.07918 |
| 926 | 0.00658 |
| 927 | 0.00251 |
| 928 | 0.00253 |
| 929 | 0.00385 |
| 930 | 0.00039 |
| 931 | 0.00135 |
| 932 | 0.01298 |
| 933 | 0.00063 |
| 934 | 0.00444 |
| 935 | 0.00704 |
| 936 | 0.13773 |
| 937 | 0.00158 |
| 938 | 0.00029 |
| 939 | 0.00341 |
| 940 | 0.00058 |
| 941 | 0.00004 |
| 942 | 0.00132 |
| 943 | 0.00009 |
| 944 | 0.00096 |
| 945 | 0.00007 |
| 946 | 0.00105 |
| 947 | 0.00912 |
| 948 | 0.00235 |
| 949 | 0.00016 |
| 950 | 0.00303 |
| 951 | 0.00028 |
| 952 | 0.00174 |
| 953 | 0.01118 |
| 954 | 0.00142 |
| 955 | 0.02311 |
| 956 | 0.00148 |
| 957 | 0.09734 |
| 958 | 0.15167 |
| 959 | 0.00744 |
| 960 | 0.00516 |
| 961 | 0.00121 |
| 962 | 0.00441 |
| 963 | 0.00395 |
| 964 | 0.00044 |
| 965 | 0.00003 |
| 966 | 0.00014 |
| 967 | 0.00018 |
| 968 | 0.00005 |
| 969 | 0.00042 |
| 970 | 0.00016 |
| 971 | 0.00277 |
| 972 | 0.08109 |
| 973 | 0.00027 |
| 974 | 0.00617 |
| 975 | 1.55107 |
| 976 | 1.78969 |
| 977 | 2.58237 |
| 978 | 2.20944 |
| 979 | 2.94208 |
| 980 | 2.52402 |
| 981 | 1.77492 |
| 982 | 4.96882 |
| 983 | 2.18111 |
| 984 | 2.76929 |
| 985 | 2.99369 |
| 986 | 2.35814 |
| 987 | 2.06069 |
| 988 | 2.06658 |
| 989 | 0.00005 |
| 990 | 0.00059 |
| 991 | 0.00203 |
| 992 | 0.00016 |
| 993 | 0.00080 |
| 994 | 0.00006 |
| 995 | 0.00274 |
| 996 | 0.00060 |

In one aspect, the present invention provides compounds of formula (I) and their pharmaceutically acceptable salts or esters as described herein, wherein said compounds of formula (I) and their pharmaceutically acceptable salts or esters have IC₅₀'s for MAGL inhibition below 25 μM, preferably below 10 μM, more preferably below 5 μM as measured in the MAGL assay described herein.

In one embodiment, compounds of formula (I) and their pharmaceutically acceptable salts or esters as described herein have IC₅₀ (MAGL inhibition) values between 0.000001 μM and 25 μM, particular compounds have IC₅₀ values between 0.000005 μM and 10 μM, further particular compounds have IC₅₀ values between 0.00005 μM and 5 μM, as measured in the MAGL assay described herein.

### Using the Compounds of the Invention

The compounds of the present invention are useful as medicaments for the treatment or prophylaxis of various diseases and disorders that are associated with monoacylglycerol lipase (MAGL).

Thus, in one aspect, the present invention provides a compound of formula (I), or a pharmaceutically acceptable salt thereof, as described herein for use as a therapeutically active substance.

In a further aspect, the present invention provides a compound of formula (I) as described herein, or a pharmaceutically acceptable salt thereof, for use in a method of inhibiting monoacylglycerol lipase in a mammal.

In a further aspect, the present invention provides a compound of formula (I) as described herein, or a pharmaceutically acceptable salt thereof, for use in the treatment or prophylaxis of diseases or disorders that are associated with monoacylglycerol lipase in a mammal.

In a further aspect, the present invention provides a method for the treatment or prophylaxis of diseases or disorders that are associated with monoacylglycerol lipase in a mammal, which method comprises administering a therapeutically effective amount of a compound of formula (I), or a pharmaceutically acceptable salt thereof, as described herein to the mammal.

In a further aspect, the present invention provides the use of a compound of formula (I), or a pharmaceutically acceptable salt thereof, as described herein in the preparation of a medicament for the treatment or prophylaxis of diseases or disorders that are associated with monoacylglycerol lipase in a mammal.

In a further aspect, the present invention provides the use of a compound of formula (I), or a pharmaceutically acceptable salt thereof, as described herein for the treatment or prophylaxis of diseases or disorders that are associated with monoacyiglycerol lipase in a mammal.

In one embodiment, said diseases or disorders that are associated with monoacylglycerol lipase are selected from neuroinflammation, neurodegenerative diseases, pain, cancer, mental disorders, and inflammatory bowel disease.

In one embodiment, said diseases or disorders that are associated with monoacylglycerol lipase are selected from multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome, visceral pain, and inflammatory bowel disease.

In one embodiment, said diseases or disorders that are associated with monoacylglycerol lipase are selected from neuroinflammation, neurodegenerative diseases, pain, cancer, mental disorders, and inflammatory bowel disease in a mammal.

In one embodiment, said diseases or disorders that are associated with monoacylglycerol lipase are selected from multiple sclerosis, Alzheimer's disease, and Parkinson's disease in a mammal.

In one embodiment, said diseases or disorders that are associated with monoacylglycerol lipase are multiple sclerosis.

In one embodiment, said diseases or disorders that are associated with monoacylglycerol lipase are selected from neuroinflammation and neurodegenerative diseases.

In one embodiment, said diseases or disorders that are associated with monoacylglycerol lipase are neurodegenerative diseases.

In one embodiment, said diseases or disorders that are associated with monoacylglycerol lipase are cancer.

In one embodiment, said diseases or disorders that are associated with monoacylglycerol lipase are inflammatory bowel disease.

In one embodiment, said diseases or disorders that are associated with monoacylglycerol lipase are pain.

### Pharmaceutical Compositions and Administration

In one aspect, the present invention provides a pharmaceutical composition comprising a compound of formula (I) as described herein and a therapeutically inert carrier.

In one embodiment, there is provided a pharmaceutical composition according to Example 941 or 942.

The compounds of formula (I) and their pharmaceutically acceptable salts and esters can be used as medicaments (e.g. in the form of pharmaceutical preparations). The pharmaceutical preparations can be administered internally, such as orally (e.g. in the form of tablets, coated tablets, dragees, hard and soft gelatin capsules, solutions, emulsions or suspensions), nasally (e.g. in the form of nasal sprays) or rectally (e.g. in the form of suppositories). However, the administration can also be effected parentally, such as intramuscularly or intravenously (e.g. in the form of injection solutions).

The compounds of formula (I) and their pharmaceutically acceptable salts and esters can be processed with pharmaceutically inert, inorganic or organic adjuvants for the production of tablets, coated tablets, dragees and hard gelatin capsules. Lactose, corn starch or derivatives thereof, talc, stearic acid or its salts etc. can be used, for example, as such adjuvants for tablets, dragees and hard gelatin capsules.

Suitable adjuvants for soft gelatin capsules are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should be appropriate. It will, however, be clear that the upper limit given herein can be exceeded when this is shown to be indicated.

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC) or crystallization.

All reaction examples and intermediates were prepared under an argon atmosphere if not specified otherwise.

### Example 1

### [6-(5-cyclopropyl-4H-1,2,4-triazol-3-yl)-2-azaspiro[3.3]heptan-2-yl]-[3-[6-[[1-(trifluoromethyl)cyclopropyl]amino]-3-pyridyl]azetidin-1-yl]methanone

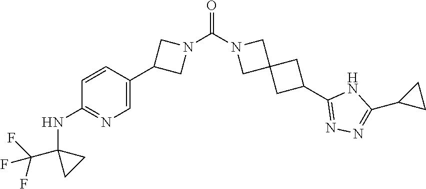 [SMILES: O=C(N1CC(c2ccc(NC3(C(F)(F)F)CC3)nc2)C1)N1CC2(CC(c3nnc(C4CC4)[nH]3)C2)C1]

To a solution of 6-(3-cyclopropyl-1H-1,2,4-triazol-5-yl)-2-azaspiro[3.3]heptane; trifluoroacetic acid (A.1) (35 mg, 0.110 mmol) in N,N-dimethylformamide (0.700 mL) cooled down to 0° C. was added DIPEA (154 μL, 0.880 mmo) followed by addition of bis(1,2,4-triazol-1-yl)methanone (19.0 mg, 0.115 mmol) after which the reaction mixture was stirred at 0° C. for 30 min to generate [6-(5-cyclopropyl-4H-1,2,4-triazol-3-yl)-2-azaspiro[3.3]heptan-2-yl]-(1,2,4-triazol-1-yl)methanone intermediate. Addition of [5-(azetidin-3-yl)-2-pyridyl]-[1-(trifluoromethyl)cyclopropyl]amine4-methylbenzene sulfonic acid (B.190) (69.5 mg, 0.115 mmol) to the reaction mixture which was then stirred at 50° C. for 18 h. The crude reaction mixture was directly submitted for reversed-phase HPLC purification to yield 21.4 mg of the desired product. MS (ESI): m/z=488.3 [M+H]⁺

### Example 3

### [6-(5-cyclopropyl-4H-1,2,4-triazol-3-yl)-2-azaspiro[3.3]heptan-2-yl]-[6-[15-(trifluoromethyl)pyrazin-2-yl]methyl]-2-azaspiro[3.3]heptan-2-yl]methanone

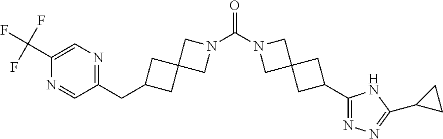 [SMILES: O=C(N1CC2(CC(Cc3cnc(C(F)(F)F)cn3)C2)C1)N1CC2(CC(c3nnc(C4CC4)[nH]3)C2)C1]

To a solution of 6-[[5-(trifluoromethyl)pyrazin-2-yl]methyl]-2-azaspiro[3.3]heptane; 4-methylbenzenesulfonic acid (B.4) (12.0 g, 27.9 mmol) in acetonitrile (114 mL) cooled down to 0° C., was added DIPEA (18.0 g, 24.4 mL, 140 mmol) followed by addition of bis(1,2,4-triazol-1-yl)methanone (4.82 g, 29.3 mmol) after which the reaction mixture was stirred at 0° C. for 30 min. 6-(3-cyclopropyl-1H-1,2,4-triazol-5-yl)-2-azaspiro[3.3]heptane; 2,2,2-trifluoroacetic acid (A.1) (11.1 g, 34.9 mmol) was added to the reaction mixture which was then stirred at 50° C. for 18 h. Volatiles were removed in vacuo, the crude residue was dissolved in 250 mL ethyl acetate and washed with 125 mL saturated aqueous NH₄Cl solution. The organic phase was collected and the aqueous phase was back-extracted with 250 mL ethyl acetate. The combined organic phases were dried over sodium sulfate and evaporated down to dryness to yield a crude residue, which was purified by flash chromatography (eluent mixture of heptane and a solution (EtOAc:EtOH 3:1) (10% to 80%) to yield a yellow gum (11.5 g) which was further purified by SFC to yield the title compound (9.12 g, 66%) as a white solid. MS (ESI): m/z=488.4 [M+H]⁺

In analogy to Example 1 and Example 3, Examples in the following table were generated, using the respective building blocks A.X and B.X. In some cases (marked with *) the reaction was carried out with isolated [6-(5-cyclopropyl-4H-1,2,4-triazol-3-yl)-2-azaspiro[3.3]heptan-2-yl]-(1,2,4-triazol-1-yl)methanone intermediate. In some cases, TEA can be used instead of DIPEA, and DMF and ACN can be used interchangeably as solvents.

Lengthy table referenced here
US20250099448A1-20250327-T00001
Please refer to the end of the specification for access instructions.

### Example 158

### [6-(5-cyclobutyl-4H-1,2,4-triazol-3 yl)-2-azaspiro[3.3]heptan-2-yl]-[6-[(2,4-difluorophenyl)methyl]-2,6-diazaspiro[3.3]heptan-2-yl]methanone

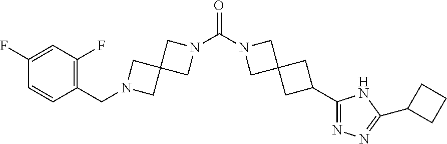 [SMILES: O=C(N1CC2(CC(c3nnc(C4CCC4)[nH]3)C2)C1)N1CC2(CN(Cc3ccc(F)cc3F)C2)C1]

To a solution of 2-[6-(2,4-difluorobenzyl)-2,6-diazaspiro[3.3]heptane-2-carbonyl]-2-azaspiro[3.3]heptane-6-carbonitrile (108 mg, 0.287 mmol) in 1-butanol (1.6 mL) was added cyclobutanecarbohydrazide (CAS: 98069-56-8) (98.3 mg, 0.861 mmol) and 1,8-diazabicyclo(5.4.0)undec-7-ene (65.6 mg, 0.431 mmol) after which the reaction was stirred at 110° C. for 18 h. A further addition of cyclobutanecarbohydrazide (98.3 mg, 0.861 mmol) and 1,8-diazabicyclo(5.4.0)undec-7-ene (65.6 mg, 0.431 mmol) was made, after which the reaction mixture was stirred at 160° C. for 20 h under microwave radiation. Volatiles were removed in vacuo. The crude residue was dissolved in DMF, which was directly submitted for reversed-phase HPLC purification to yield the title compound (39.7 mg). MS (ESI): m/z=469.4 [M+H]⁺

### Step a) 2-(1,2,4-triazole-1-carbonyl)-2-azaspiro[3.3]heptane-6-carbonitrile

To a solution of tert-butyl 6-cyano-2-azaspiro[3.3]heptane-2-carboxylate (CAS: 1374658-94-2) (1.0 g, 4.41 mmol) in dichloromethane (5 mL) was added TFA (1.02 mL, 13.2 mmol) and the reaction mixture was stirred at RT for 18 h. The reaction mixture was then diluted with 10 mL of dichloromethane and cooled down to 0° C., followed by addition of DIPEA (4.62 mL, 13.2 mmol) and bis(1,2,4-triazol-1-yl)methanone (760 mg, 4.63 mmol). The reaction mixture was then stirred at 0° C. for 1 h and at RT for 18 h. The reaction was then diluted with dichloromethane and washed with aq. Na₂CO₃ 1 M solution. The organic phase was collected and the aqueous phase was back-extracted with dichloromethane. The combined organic phases were dried over sodium sulfate and evaporated down to dryness to yield 1120 mg of the crude title compound (purity roughly 85%) which was used without further purification. MS (ESI): m/z=218.1 [M+H]⁺

### Step b) 2-[6-(2,4-difluorobenzyl)-2,6-diazaspiro[3.3]heptane-2-carbonyl)-2-azaspiro[3.3]heptane-6-carbonitrile

To a solution of 2-(1,2,4-triazole-1-carbonyl)-2-azaspiro[3.3]heptane-6-carbonitrile (1.15 g, 5.29 mmol) in acetonitrile (25 mL) was added DIPEA (5.55 mL, 31.8 mL) and 2-(2,4-difluorobenzyl)-2,6-diazaspiro[3.3]heptane; trifluoroacetic acid (3.87 g, 5.82 mmol) after which the reaction mixture was stirred at 50° C. for 18 h. Volatiles were removed in vacuo and the crude residue was partitioned between ethyl acetate and aq. Na₂CO₃ 1 M solution. The organic phase was collected and the aqueous phase was back-extracted with ethyl acetate. The combined organic phases were dried over sodium sulfate and evaporated down to dryness. The crude material was purified by flash chromatography with a 120 g SiO₂ column, using an eluent mixture of dichloromethane and methanol (0% to 10%) to yield 1.35 g of the title compound. MS (ESI): m/z=373.3 [M+H]⁺

### Example 172

### 2-[[2-[6-(5-cyclopropyl-4H-1,2,4-triazol-3-yl)-2-azaspiro[3.3]heptane-2-carbonyl]-2,6-diazaspiro[3.3]heptan-6-yl]methyl]benzoic Acid

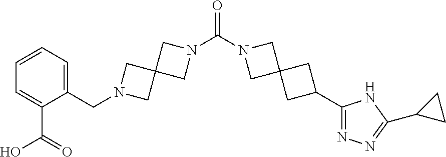 [SMILES: O=C(O)c1ccccc1CN1CC2(C1)CN(C(=O)N1CC3(CC(c4nnc(C5CC5)[nH]4)C3)C1)C2]

A solution of methyl 2-[[2-[6-(5-cyclopropyl-4H-1,2,4-triazol-3-yl)-2-azaspiro[3.3]heptane-2-carbonyl]-2,6-diazaspiro[3.3]heptan-6-yl]methyl]benzoate (Example 171) (150 mg, 0.315 mmol) in 5 M NaOH 5.0 M aqueous solution (189 μL, 0.944 mmol) was stirred at 50°c for 18 h. Volatiles were removed in vacuo, the obtained crude residue was dissolved in DMF and submitted for reversed-phase HPLC purification to yield 61.8 mg of the title compound. MS (ESI⁻): m/z=461.4 [M−H]⁻

### Example 217

### 1-[2-[6-(5-cyclopropyl-4H-1,2,4-triazol-3-yl)-2-azaspiro[3.3]heptane-2-carbonyl]-2,6-diazaspiro[3.3]heptan-6-yl]-2,2,2-trifluoro-ethanone

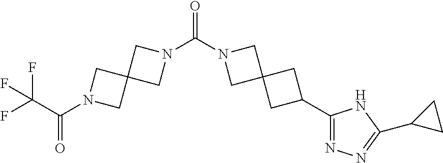 [SMILES: O=C(N1CC2(CC(c3nnc(C4CC4)[nH]3)C2)C1)N1CC2(C1)CN(C(=O)C(F)(F)F)C2]

To a solution of 6-[6-(5-cyclopropyl-4H-1,2,4-triazol-3-yl)-2-azaspiro[3.3]heptane-2-carbonyl]-2,6-diazaspiro[3.3]heptane-2-carboxylic acid tert-butyl ester (555 mg, 1.17 mmol) in dichloromethane (4.5 mL) was added TFA (898 μL, 11.7 mmol) and the reaction mixture was stirred at room temperature for 18 h. Volatiles were removed in vacuo to a residue that was submitted for reversed-phase HPLC purification to yield 208 mg of the title compound (as a side product of the originally intended reaction). MS (ESI⁻): m/z=425.3 [M+H]⁺

### Step a) 1-[2-[6-(5-cyclopropyl-4H-1,2,4-triazol-3-yl)-2-azaspiro[3.3]heptane-2-carbonyl]-2,6-diazaspiro[3.3]heptan-6 yl]-2,2,2-trifluoro-ethanone

To a solution of 2,6-diazaspiro[3.3]heptane-2-carboxylic acid tert-butyl ester; oxalic acid (519 mg, 1.07 mmol) in N,N-dimethylformamide (10 mL) cooled down to 0° C. was added DIPEA (1.38 g, 1.86 mL, 10.7 mmol) and bis(1,2,4-triazol-1-yl)methanone (0.350 g, 2.13 mmol) after which the reaction mixture was stirred at 0° C. for 30 min. Addition of 6-(5-cyclopropyl-H-1,2,4-triazol-3-yl)-2-azaspiro[3.3]heptane; trifluoroacetic acid (A.1) (1.36 g, 2.13 mmol) to the reaction mixture which was then stirred at 100° C. for 18 h. Volatiles were removed in vacuo, the crude residue was then partitioned between ethyl acetate and sat. aq. sol. NH₄Cl solution. The organic phase was collected and the aqueous phase was back-extracted with ethyl acetate. The combined organic phases were dried over sodium sulfate and evaporated down to dryness. The crude material was purified by flash chromatography (eluent mixture of dichloromethane and methanol (0% to 10%)) to yield the title compound (555 mg). MS (ESI): m/z=429.4 [M+H]⁺

### Example 303

### [6-(5-cyclopropyl-4H-1,2,4-triazol-3-yl)-2-azaspiro[3.3]heptan-2-yl]-[3-[6-(2-oxa-7-azaspiro[3.4]octan-7 yl)-3-pyridyl]azetidin-1-yl]methanone

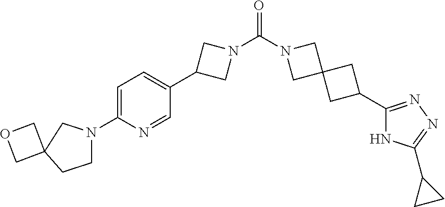 [SMILES: O=C(N1CC(c2ccc(N3CCC4(COC4)C3)nc2)C1)N1CC2(CC(c3nnc(C4CC4)[nH]3)C2)C1]

To a suspension of [3-(6-bromo-3-pyridyl)azetidin-1-yl]-[6-(5-cyclopropyl-4H-1,2,4-triazol-3-yl)-2-azaspiro[3.3]heptan-2-yl]methanone (50 mg, 0.102 mmol) and 2-oxa-7-azaspiro[3.4]octane; oxalic acid (20.62 mg, 0.102 mmol) in 1,4-dioxane (0.636 mL) under argon were added PdCl(crotyl)AmPhos (2.5 mg, 0.005 mmol), 2 M sodium tert-butoxide (2 M in THF) (152.25 μL, 0.305 mmol), and the mixture was heated in a microwave oven at 80° C. for 3 h. The mixture was filtered and the filtrate was evaporated. Purification by RP-HPLC gave the title compound (0.002 g, 5.18%) as a colorless solid. MS (ESI): m/z=474.3 [M−H]⁻

### Step a): 3-(6-bromo-3 pyridyl)azetidin-1-yl]-[6-(5-cyclopropyl-4H-1,2,4-triazol-3 yl)-2-azaspiro[3.3]heptan-2 yl]methanone

A solution of 6-(5-cyclopropyl-4H-1,2,4-triazol-3-yl)-2-azaspiro[3.3]heptane; 2,2,2-trifluoroacetic acid (149.4 mg, 0.469 mmol), CDT (76.97 mg, 0.469 mmol) and Et₃N (326.89 μL, 2.35 mmol) in ACN (1.7 mL) was stirred for 3 h at 23° C., before being treated with 5-(azetidin-3-yl)-2-bromo-pyridine (CAS RN: 1260681-59-1; 150.6 mg, 0.391 mmol). The mixture was heated for 18 h at 50° C., before being evaporated. Purification by FC(SiO₂; DCM/MeOH) gave the title compound (0.159 g; 82.6%) as a light yellow oil. MS (ESI): m/z=443.1 [M+H]⁺

### Example 304 and Example 305

### Trans-[6-(5-Cyclopropyl-4H-1,2,4-triazol-3-yl)-2-azaspiro[3.3]heptan-2-yl]-[3-[4-[3-(difluoromethyl)cyclobutyl]phenyl]azetidin-1-yl]methanone (Example 304)

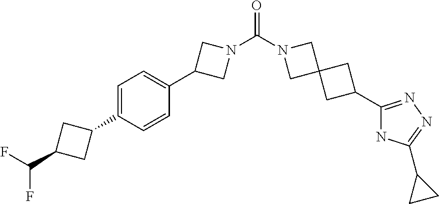 [SMILES: O=C(N1CC(c2ccc([C@H]3C[C@H](C(F)F)C3)cc2)C1)N1CC2(CC(c3nnc(C4CC4)[nH]3)C2)C1]

### cis-[6-(5-Cyclopropyl-4H-1,2,4-triazol-3-yl)-2-azaspiro[3.3]heptan-2-yl]-[3-[4-[3-(difluoromethyl)cyclobutyl]phenyl]azetidin-1-yl]methanone (Example 305)

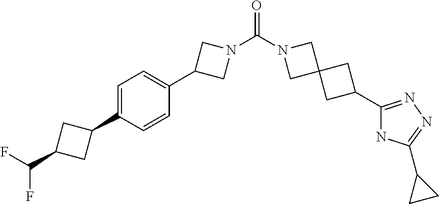 [SMILES: O=C(N1CC(c2ccc([C@H]3C[C@@H](C(F)F)C3)cc2)C1)N1CC2(CC(c3nnc(C4CC4)[nH]3)C2)C1]

The mixture of [6-(5-cyclopropyl-4H-1,2,4-triazol-3-yl)-2-azaspiro[3.3]heptan-2-yl]-[3-[4-[3-difluoromethyl)cyclobutyl]phenyl]azetidin-1-yl]methanone was separated on a SFC column (chiral 4-Cellulose) using 30% of methanol to get the desired products was colorless solids. MS (ESI): m/z=468.4 [M+H]⁺.

trans-[6-(5-Cyclopropyl-4H-1,2,4-triazol-3-yl)-2-azaspiro[3.3]heptan-2-yl]-[3-[4-[3-(difluoromethyl)cyclobutyl]phenyl]azetidin-1-yl]methanone (Example 304) is the second eluting isomer.

cis-[6-(5-Cyclopropyl-4H-1,2,4-triazol-3-yl)-2-azaspiro[3.3]heptan-2-yl]-[3-[4-[3-(difluoromethyl)cyclobutyl]phenyl]azetidin-1-yl]methanone (Example 305) is the first eluting isomer.

### Step a) Tert-Butyl 3-[4-[3-(difluoromethyl)cyclobutyl]phenyl]azetidine-1-carboxylate

In a sealed and argon filled tube were combined tert-butyl 3-(4-bromophenyl)azetidine-1-carboxylate (CAS RN: 1203681-52-0; 470 mg, 1.51 mmol), dichloronickel; 1,2-dimethoxyethane (CAS RN: 29046-78-4; 33.08 mg, 0.151 mmol), bis[3,5-difluoro-2-[5-(trifluoromethyl)-2-pyridyl]phenyl]iridium(1+); 4-tert-butyl-2-(4-tert-butyl-2-pyridyl)pyridine; hexafluorophosphate (CAS RN: 870987-63-6; 16.89 mg, 0.015 mmol) and 4-tert-butyl-2-(4-tert-butyl-2-pyridyl)pyridine (CAS RN: 72914-19-3; 40.41 mg, 0.151 mmol) and the mixture was suspended in ethylene glycol dimethyl ether (12.5 mL) before addition of bis(trimethylsilyl)silyl-trimethyl-silane (CAS RN: 1873-77-4; 464.45 μL, 1.51 mmol) and 2,6-dimethylpyridine (350.67 μL, 3.01 mmol). After degassing with Ar, 1-bromo-3-(difluoromethyl)cyclobutane (CAS RN: 2241140-79-2; 334.23 mg, 1.81 mmol) was added and the suspension was stirred in the Penn photoreactor with a 420 nm lamp (50% intensity) for 8 h, before being filtered and evaporated. Purification by FC (SiO₂; heptane/EtOAc) gave the title compound (0.110 g; 20.1%) as a colorless oil. MS (ESI): m/z=282.2 [M-tBu+H]⁺

### Step b) 3-[4-[3-(Difluoromethyl)cyclobutyl]phenyl]azetidine; 4-methylbenzenesulfonic Acid

A solution of tert-butyl 3-[4-[3-(difluoromethyl)cyclobutyl]phenyl]azetidine-1-carboxylate (110 mg, 0.326 mmol) and p-toluenesulfonic acid monohydrate (68.22 mg, 0.359 mmol) in ethyl acetate (0.6 mL) was stirred at reflux for 60 min. A suspension formed which was filtered. The filter cake was washed with a small volume of ethyl acetate, to give the title compound (0.10 g, 70.4%) as a colorless solid. MS (ESI): m/z=238.2 [M+H]⁺

### Step c) [6-(5-Cyclopropyl-4H-1,2,4-triazol-3-yl)-2-azaspiro[3.3]heptan-2 yl]-[3-[4-[3-(difluoromethyl)cyclobutyl]phenyl]azetidin-1-yl]methanone

To a suspension of 6-(5-cyclopropyl-4H-1,2,4-triazol-3-yl)-2-azaspiro[3.3]heptane; 2,2,2-trifluoroacetic acid (120.56 mg, 0.379 mmol) and CDI (62.16 mg, 0.379 mmol) in acetonitrile (0.900 mL) was added TEA (191.97 μL, 1.38 mmol) and the mixture was stirred at 23° C. for 30 min. To the clear and colorless solution was added 3-[4-[3-(difluoromethyl)cyclobutyl]phenyl]azetidine 0.1:1 tosylic acid (150 mg, 0.344 mmol) and the solution was stirred at 50° C. for 22 h, before being evaporated. Purification by RP-HPLC gave the title compound (0.065 g; 39.5%) as a colorless gum. MS (ESI): m/z=468.3 [M+H]⁺

### Example 306

### [6-(5-Cyclopropyl-4H-1,2,4-triazol-3 yl)-2-azaspiro[3.3]heptan-2-yl]-[3-hydroxy-3-[4-[3-(trifluoromethyl)azetidin-1-yl]phenyl]azetidin-1 yl)methanone

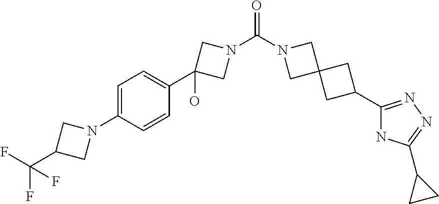 [SMILES: O=C(N1CC2(CC(c3nnc(C4CC4)[nH]3)C2)C1)N1CC(O)(c2ccc(N3CC(C(F)(F)F)C3)cc2)C1]

To a suspension of [3-(4-bromophenyl)-3-hydroxy-azetidin-1-yl]-[6-(5-cyclopropyl-4H-1,2,4-triazol-3-yl)-2-azaspiro[3.3]heptan-2-yl]methanone (127 mg, 0.230 mmol) and 3-(trifluoromethyl)azetidine (28.77 mg, 0.230 mmol) in 1,4-dioxane (1.4 mL) under argon were added PdCl(crotyl)AmPhos (CAS RN: 1334497-06-1; 5.67 mg, 0.011 mmol), 2 M sodium tert-butoxide (2 M in THF) (344.97 μL, 0.690 mmol), and the mixture was heated in a microwave oven at 80° C. for 30 minutes. Another batch of 3-(trifluoromethyl)azetidine (57.54 mg, 0.460 mmol) was added and the mixture was heated in the microwave oven to 110° C. for 30 min. Another batch of 3-(trifluoromethyl)azetidine (57.54 mg, 0.460 mmol) and 2 M sodium tert-butoxide (2 M in THF) (229.98 μL, 0.460 mmol) were added and the mixture was heated in the microwave to 120° C. for 30 min. Purification by RP-HPLC gave the title compound (0.012 g; 10.3%) as a colorless solid. MS (ESI): m/z=503.3 [M+H]⁺

### Step a) [3-(4-Bromophenyl)-3-hydroxy-azetidin-1 yl]-[6-(5-cyclopropyl-4H-1,2,4-triazol-3 yl)-2-azaspiro[3.3]heptan-2-yl]methanone

To a suspension of 6-(5-cyclopropyl-4H-1,2,4-triazol-3-yl)-2-azaspiro[3.3]heptane; 2,2,2-trifluoroacetic acid (198.52 mg, 0.624 mmol) and CDI (102.36 mg, 0.624 mmol) in acetonitrile (1.5 mL) was added TEA (316.11 μL, 2.27 mmol) and the mixture was stirred at 23° C. for 1.5 h. To the clear and colorless solution was added 3-(4-bromophenyl)azetidin-3-ol; hydrochloride (CAS RN: 1989671-90-0; 150 mg, 0.567 mmol) and the solution was stirred at 50° C. for 16 h. The mixture was evaporated. Purification by FC (SiO₂; heptane/EtOAc/EtOH) gave the title compound (0.127 g, 40.5%) as a colorless foam. MS (ESI): m/z=458.1 [M+H]⁺

### Example 387

### 2-[3-[1-[6-(5-cyclopropyl-4H-1,2,4-triazol-3-yl)-2-azaspiro[3.3]heptane-2-carbonyl]azetidin-3-yl]oxyphenyl]-2-methyl-propanoic Acid

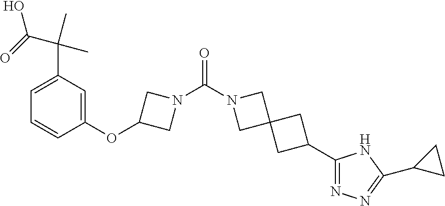 [SMILES: CC(C)(C(=O)O)c1cccc(OC2CN(C(=O)N3CC4(CC(c5nnc(C6CC6)[nH]5)C4)C3)C2)c1]

To a solution of Example 318 (80 mg, 0.117 mmol) in MeOH (1 mL) was added 5 M NaOH aqueous solution (70.06 μL, 0.350 mmol). The mixture was stirred for 18 h at 50° C., before being evaporated. Purification by RP-HPLC gave the title compound (12.3 mg, 22.2% yield). MS (ESI): m/z=466.5 [M+H]⁺

### Examples 388 and 389

### [3-[[2-fluoro-4-(trifluoromethyl)phenyl]methoxy]azetidin-1-yl]-[6-[5-[(1R)-2,2,2-trifluoro-1-hydroxy-ethyl]-4H-1,2,4-triazol-3-yl]-2-azaspiro[3.3]heptan-2-yl]methanone (Example 388) and [3-[[2-fluoro-4-(trifluoromethyl)phenyl]methoxy]azetidin-1-yl]-[6-[5-[(1S)-2,2,2-trifluoro-1-hydroxy-ethyl]-4H-1,2,4-triazol-3 yl]-2-azaspiro[3.3]heptan-2-yl]methanone (Example 389)

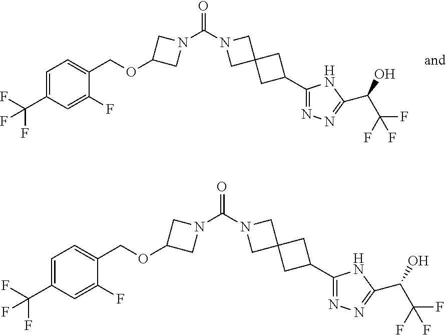 [SMILES: O=C(N1CC(OCc2ccc(C(F)(F)F)cc2F)C1)N1CC2(CC(c3nnc([C@@H](O)C(F)(F)F)[nH]3)C2)C1 ; O=C(N1CC(OCc2ccc(C(F)(F)F)cc2F)C1)N1CC2(CC(c3nnc([C@H](O)C(F)(F)F)[nH]3)C2)C1]

Example 336 (62.9 mg, 0.12 mmol) was purified by chiral chromatography, to give Example 388 (22.6 mg, 19.8% yield) as the first eluting compound and Example 389 (25.4 mg, 22.3% yield) as the second eluting compound. MS (ESI): m/z=538.3 [M+H]⁺ for both enantiomers

### Example 390

### 2-[4-[1-[6-(5-cyclopropyl-4H-1,2,4-triazol-3-yl)-2-azaspiro[3.3]heptane-2-carbonyl]azetidin-3-yl]phenyl]benzoic Acid

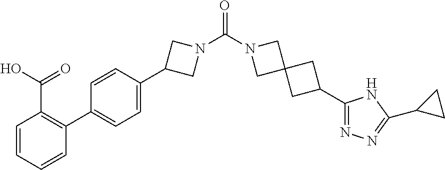 [SMILES: O=C(O)c1ccccc1-c1ccc(C2CN(C(=O)N3CC4(CC(c5nnc(C6CC6)[nH]5)C4)C3)C2)cc1]

To a solution of Example 339 (75 mg, 0.143 mmol) in MeOH (1 mL) was added 5 M aqueous NaOH solution (85.91 μL, 0.430 mmol). The reaction mixture was stirred for 18 h at 50° C., before being evaporated and purified by RP-HPLC, to give the title compound (51.3 mg, 70.4% yield). MS (ESI): m/z=484.3 [M+H]⁺

### Example 391

### 2-[4-[1-[6-(5-cyclopropyl-4H-1,2,4-triazol-3-yl)-2-azaspiro[3.3]heptane-2-carbonyl]azetidin-3 yl]phenyl]-N-ethyl-benzamide

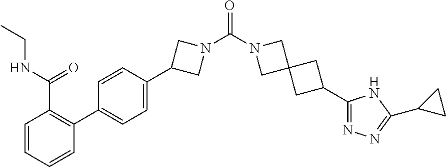 [SMILES: CCNC(=O)c1ccccc1-c1ccc(C2CN(C(=O)N3CC4(CC(c5nnc(C6CC6)[nH]5)C4)C3)C2)cc1]

To a solution of Example 390 (45 mg, 0.088 mmol) in DMF (1 mL) were added DIPEA (108.09 μL, 0.619 mmol) and HATU (35.3 mg, 0.093 mmol), at 23° C. The mixture was stirred for 30 min at this temperature, before being treated with ethylamine; hydrochloride (21.63 mg, 0.265 mmol). The mixture was stirred for another 18 h, before being purified by RP-HPLC, to give the title compound (40.8 mg, 85.9% yield). MS (ESI): m/z=511.4 [M+H]⁺

### Example 392

### 1-[4 [1-[6-(5-cyclopropyl-4H-1,2,4-triazol-3-yl)-2-azaspiro[3.3]heptane-2-carbonyl]azetidin-3-yl]phenyl]cyclopropanecarboxylic Acid

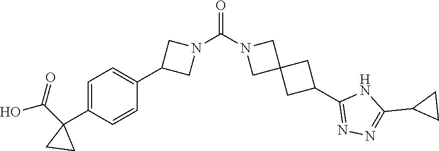 [SMILES: O=C(N1CC(c2ccc(C3(C(=O)O)CC3)cc2)C1)N1CC2(CC(c3nnc(C4CC4)[nH]3)C2)C1]

To a solution of Example 372 (90 mg, 0.185 mmol) in MeOH (I mL) was added 5 M NaOH (111.14 μL, 0.556 mmol). The mixture was stirred for 18 h at 50° C., before being evaporated. Purification by RP-HPLC gave the title compound (43.5 mg, 49.8%). MS (ESI): m/z=448

3 [M+H]⁺

### Example 393

### 1-[4-[1-[6-(5-cyclopropyl-4H-1,2,4-triazol-3-yl)-2-azaspiro[3.3]heptane-2-carbonyl]azetidin-3-yl]phenyl]cyclopropanecarboaamide

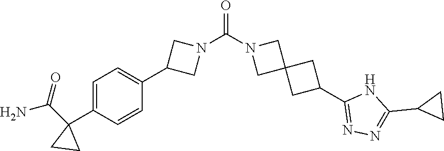 [SMILES: NC(=O)C1(c2ccc(C3CN(C(=O)N4CC5(CC(c6nnc(C7CC7)[nH]6)C5)C4)C3)cc2)CC1]

To a solution of Example 392 (38 mg, 0.085 mmol,) in DMF (1 mL) was added DIPEA (118.53 μL, 0.679 mmol) and HATU (33.9 mg, 0.089 mmol), at 23° C. The mixture was stirred for 20 min at this temperature, before being treated with ammonium chloride (22.71 mg, 0.425 mmol). The mixture was stirred for another 18 h before being directly purified by RP-HPLC, to give the title compound (28.6 mg, 67.9%). MS (ESI): m/z=447.3 [M+H]⁺

### Example 394

### (2S)-1-[4-[1-[6-(5-cyclopropyl-4H-1,2,4-triazol-3-yl)-2-azaspiro[3.3]heptane-2-carbonyl]azetidin-3 yl]phenyl]piperidine-2-carboxamide

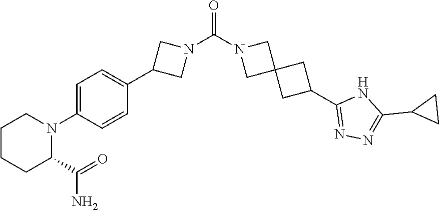 [SMILES: NC(=O)[C@@H]1CCCCN1c1ccc(C2CN(C(=O)N3CC4(CC(c5nnc(C6CC6)[nH]5)C4)C3)C2)cc1]

Example 384 (45.7 mg, 0.089 mmol) was separated by chiral SFC, to give the title compound (21.8 mg, 45.2% yield). MS (ESI): m/z=490.3 [M+H]⁺

### Examples 395 and 396

### (2S)-1-[4-[1-[6-(5-cyclopropyl-4H-1,2,4-triazol-3-yl)-2-azaspiro[3.3]heptane-2-carbonyl]azetidin-3-yl]phenyl]-4,4-difluoro-piperidine-2-carboxamide (Example 395) and (2R)-1-[4-[1-[6-(5-cyclopropyl-4H-1,2,4-triazol-3-yl)-2-azaspiro[3.3]heptane-2-carbonyl]azetidin-3-yl]phenyl]-4,4-difluoro-piperidine-2-carboxamide (Example 396)

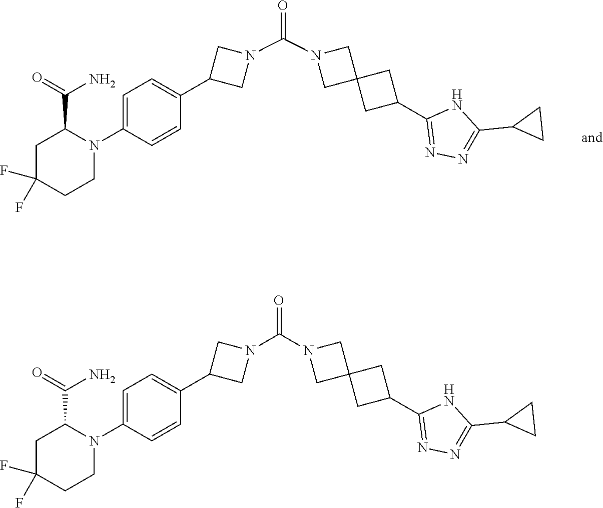 [SMILES: NC(=O)[C@@H]1CC(F)(F)CCN1c1ccc(C2CN(C(=O)N3CC4(CC(c5nnc(C6CC6)[nH]5)C4)C3)C2)cc1 ; NC(=O)[C@H]1CC(F)(F)CCN1c1ccc(C2CN(C(=O)N3CC4(CC(c5nnc(C6CC6)[nH]5)C4)C3)C2)cc1]

Example 385 (36.2 mg, 0.065 mmol) was separated by chiral SFC, to give Example 88 (12.1 mg, 33.4°% yield) and Example 89 (12.4 mg, 34.2% yield). MS (ESI): m/z=526.3 [M+H]⁺ for both.

### Examples 397 and 398

### (2S)-1-[4-[1-[6-(5-cyclopropyl-4H-1,2,4-triazol-3-yl)-2-azaspiro[3.3]heptane-2-carbonyl]azetidin-3-yl]phenyl]pyrrolidine-2-carboxamide (Example 397) and (2R)-1 [4-[1-[6-(5-cyclopropyl-4H-1,2,4-triazol-3-yl)-2-azaspiro[3.3]heptane-2-carbonyl]azetidin-3-yl]phenyl]pyrrolidine-2-carboxamide (Example 398)

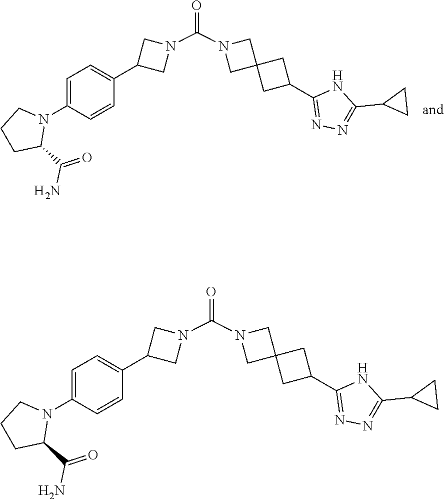 [SMILES: NC(=O)[C@@H]1CCCN1c1ccc(C2CN(C(=O)N3CC4(CC(c5nnc(C6CC6)[nH]5)C4)C3)C2)cc1 ; NC(=O)[C@H]1CCCN1c1ccc(C2CN(C(=O)N3CC4(CC(c5nnc(C6CC6)[nH]5)C4)C3)C2)cc1]

Example 386 (24.6 mg, 0.049 mmol) was separated by chiral SFC, to give Example 397 (6.8 mg, 27.9% yield) and Example 398 (7.2 mg, 29.3% yield). MS (ES): m/z=476.3 [M+H]⁺ for both.

### Example 473

### [6-(3-cyclopropyl-1H-1,2,4-triazol-5-yl)-2-azaspiro[3.3]heptan-2-yl]-[3-[[5-(trifluoromethyl)pyrazin-2-yl]amino]-1-bicyclo[1.1.1]pentanyl]methanone

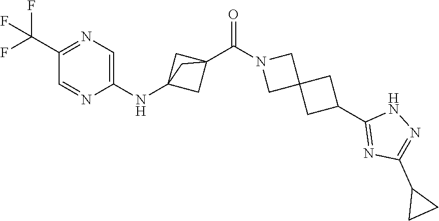 [SMILES: O=C(N1CC2(CC(c3nc(C4CC4)n[nH]3)C2)C1)C12CC(Nc3cnc(C(F)(F)F)cn3)(C1)C2]

A solution of 3-[[5-(trifluoromethyl)pyrazin-2-yl]amino]bicyclo[1.1.1]pentane-1-carboxylic acid (45 mg, 148.24 umol) in N,N-dimethylformamide (1 mL) was treated with DIPEA (76.57 mg, 103.47 uL, 592.95 umol) and HATU (59.18 mg, 155.65 umol), at 23° C. The mixture was stirred for 5 min at this temperature, before being treated with 6-(3-cyclopropyl-1H-1,2,4-triazol-5-yl)-2-azaspiro[3.3]heptane; 2,2,2-trifluoroacetic acid (A.1; 56.62 mg, 177.89 umol). The mixture was stirred at 23° C. for 18 h. Purification by RP-HPLC gave the title compound (45.6 mg, 64%) as a colorless gum. MS (ESI): m/z=460.3 [M+H]⁺

### Step a): 3-[[5-(trifluoromethyl)pyrazin-2 yl]amino]bicyclo[1.1.1]pentane-1-carboxylic Acid

A solution of 3-aminobicyclo[1.1.1]pentane-1-carboxylic acid methyl ester; hydrochloride (CAS RN: 676371-65-6; 400 mg, 2.25 mmol) and DIPEA (825.17 uL, 4.73 mmol) in N,N-dimethylformamide, extra dry (10.5 mL) was treated with 2-fluoro-5-(trifluoromethyl)pyrazine (CAS RN: 1220799-65-4; 523.59 mg, 3.15 mmol), at 0° C. The mixture was stirred for 10 min at this temperature and for 18 h at 23° C., before being evaporated. The residue was dissolved in MeOH (10.5 mL) and treated with 5 M aqueouse NaOH (2.7 mL, 13.51 mmol). The mixture was stirred for 2 h at 23° C. and evaporated. The residue was partitioned between EtOAc and 1 M aqueous HCl, and the organic layer was collected. The aqueous phase was extracted with EtOAc. The combined organic layers were dried over Na2SO4, filtered and evaporated, to give the crude title compound (642 mg, 94%) as a yellow solid. MS (ESI): m/z=274.1 [M+H]⁺

### Example 811

### [6-(5-cyclopropyl-4H-1,2,4-triazol-3-yl)-2-azaspiro[3.3]heptan-2-yl]-[4-[[5-(trifluoromethyl)pyrazin-2-yl]amino]cuban-1-yl]methanone

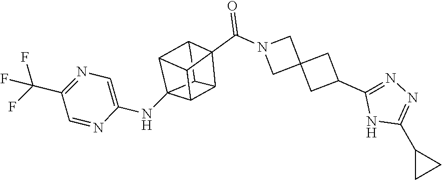 [SMILES: O=C(N1CC2(CC(c3nnc(C4CC4)[nH]3)C2)C1)C12C3CC4C1C1C2C3C41Nc1cnc(C(F)(F)F)cn1]

To a solution of 4-[[5-(trifluoromethyl)pyrazin-2-yl]amino]cubane-1-carboxylic acid (B.553) (15 mg, 0.046 mmol) in N,N-dimethylformamide (0.461 mL) was added DIEA (41.7 mg, 56.3 μL, 0.323 mmol) and HATU (26.3 mg, 0.069 mmol). Then, after pre-stirring for 5 minutes, 6-(5-cyclopropyl-4H-1,2,4-triazol-3-yl)-2-azaspiro[3.3]heptane; 2,2,2-trifluoroacetic acid (A.1) (22.0 mg, 0.069 mmol) was added in one portion. The reaction mixture was allowed to stir for 1 h, the reaction mixture was concentrated under reduced pressure and purified by reverse phase HPLC (80% MeCN in H₂O) to afford the title compound (15.2 mg, 63.2%) as white solid. MS (ESI): m/z=496.2 [M+H]⁺; ¹H NMR (300 MHz, METHANOL-d4) δ=8.35 (s, 1H), 8.07 (s, 1H), 7.91 (s, 1H), 4.39 (s, 1H), 4.27-4.12 (m, 8H), 4.00 (s, 1H), 3.52-3.43 (m, 1H), 2.70-2.59 (m, 2H), 2.58-2.47 (m, 2H), 2.08-1.97 (m, 1H), 1.11-1.01 (m, 2H), 0.99-0.92 (m, 2H).

### Example 948

### [6-[5-(1-aminocyclopropyl)-4H-1,2,4-triazol-3-yl]-2-azaspiro[3.3]heptan-2-yl]-[6-[[5-(trifluoromethyl)-2-pyridyl]methyl]-2-azaspiro[3.3]heptan-2-yl]methanone

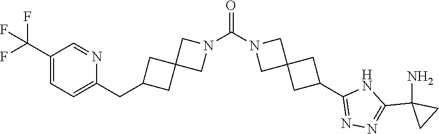 [SMILES: NC1(c2nnc(C3CC4(C3)CN(C(=O)N3CC5(CC(Cc6ccc(C(F)(F)F)cn6)C5)C3)C4)[nH]2)CC1]

To a solution of N-[1-[5-(2-azaspiro[3.3]heptan-6-yl)-1H-1,2,4-triazol-3-yl]cyclopropyl]carbamic acid tert-butyl ester; acetic acid (A.9) (140 mg, 295 μmol) in N,N-dimethylformamide (1.72 mL) cooled down to 0° C. was added DIPEA (267 mg, 361 μL, 2.07 mmol) followed by addition of bis(1,2,4-triazol-1-yl)methanone (50.9 mg. 310 μmol) after which the reaction mixture was stirred at 0° C. for 30 min. Addition of 6-[[5-(trifluoromethyl)-2-pyridyl]methyl]-2-azaspiro[3.3]heptane; 4-methylbenzenesulfonic acid (B.69) (204 mg, 339 μmol) to the reaction mixture which was then stirred at 50° C. for 18 h. Volatiles were removed in vacuo and the crude residue was dissolved in dichloromethane (1.66 mL) followed by addition of TFA (673 mg, 455 μL, 5.9 mmol) after which the reaction mixture was stirred at RT for 18 h. Volatiles were removed in vacuo, the crude residue was dissolved in DMF and the crude solution was directly submitted for a purification by reversed-phase HPLC to give a colorless gum which was further purified by SFC to give the title compound (7.5 mg) as a white solid. MS (ESI): m/z=502.4 [M+H]⁺

In analogy to Example 948, Examples in the following table were generated, using the respective building blocks A.9 and B.X.

| | | | | MS, |
|---|---|---|---|---|
| | | | Building | ESI: |
| Ex. | Structure | Systematic Name | Blocks | m/z |
| 474 | 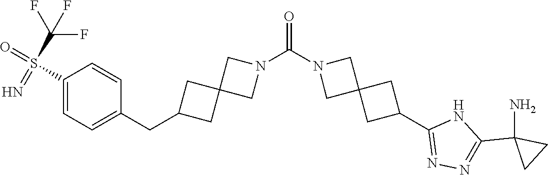 [SMILES: N=[S@](=O)(c1ccc(CC2CC3(C2)CN(C(=O)N2CC4(CC(c5nnc(C6(N)CC6)[nH]5)C4)C2)C3)cc1)C(F)(F)F] | [6-[5-(1- aminocyclopropyl)-4H- 1,2,4-triazol-3-yl]-2- azaspiro[3.3]heptan-2- yl]-[6-[[4- (trifluoromethyl- sulfonimidoyl) phenyl]methyl]- 2-azaspiro[3.3]heptan- 2-yl]methanone | A.9 and B.313 | 564.4 [M + H]⁺ |
| 636 | 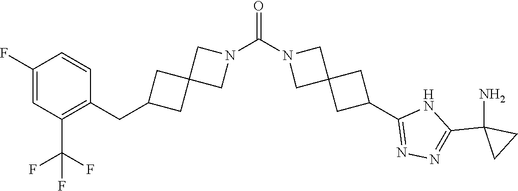 [SMILES: NC1(c2nnc(C3CC4(C3)CN(C(=O)N3CC5(CC(Cc6ccc(F)cc6C(F)(F)F)C5)C3)C4)[nH]2)CC1] | [6-[5-(1- aminocyclopropyl)-4H- 1,2,4-triazol-3-yl]-2- azaspiro[3.3]heptan-2- yl]-[6-[4-fluoro-2- (trifluoromethyl)phenyl] methyl]-2- azaspiro[3.3]heptan-2- yl]methanone | A.9 and B.17 | 519.4 [M + H]⁺ |
| 852 | 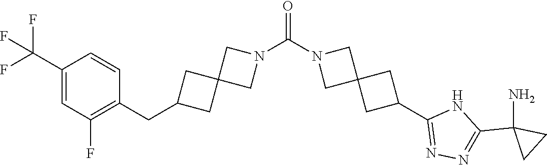 [SMILES: NC1(c2nnc(C3CC4(C3)CN(C(=O)N3CC5(CC(Cc6ccc(C(F)(F)F)cc6F)C5)C3)C4)[nH]2)CC1] | [6-[5-(1- aminocyclopropyl)-4H- 1,2,4-triazol-3-yl]-2- azaspiro[3.3]heptan-2- yl]-[6-[[2-fluoro-4- (trifluoromethyl)phenyl] methyl]-2- azaspiro[3.3]heptan-2- yl]methanone | A.9 and B.19 | 519.4 [M + H]⁺ |
| 854 | 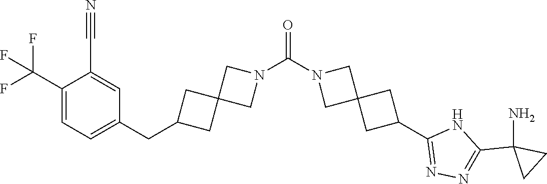 [SMILES: N#Cc1cc(CC2CC3(C2)CN(C(=O)N2CC4(CC(c5nnc(C6(N)CC6)[nH]5)C4)C2)C3)ccc1C(F)(F)F] | 5-[[2-[6-[5-(1- aminocyclopropyl)-4H- 1,2,4-triazol-3-yl]-2- azaspiro[3.3]heptane-2- carbonyl]-2- azaspiro[3.3]heptan-6- yl]methyl]-2- (trifluoromethyl) benzonitrile | A.9 and B.13 | 519.4 [M + H]⁺ |
| 855 | 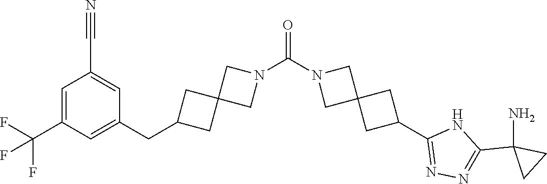 [SMILES: N#Cc1cc(CC2CC3(C2)CN(C(=O)N2CC4(CC(c5nnc(C6(N)CC6)[nH]5)C4)C2)C3)cc(C(F)(F)F)c1] | 3-[[2-[6-[5-(1- aminocyclopropyl)-4H- 1,2,4-triazol-3-yl]-2- azaspiro[3.3]heptane-2- carbonyl]-2- azaspiro[3.3]heptan-6- yl]methyl]-5- (trifluoromethyl) benzonitrile | A.9 and B.306 | 519.4 [M + H]⁺ |

The following molecules were generated in analogy to the molecules already described:

| | | | MS, |
|---|---|---|---|
| | | | ESI: |
| Ex. | Structure | Systematic Name | m/z |
| 414 | 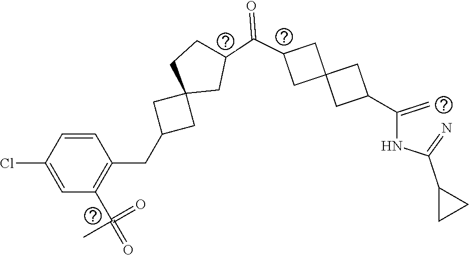 | [2-(4-chloro-2-mesyl-benzyl)-6- azaspiro[3.4]octan-6-yl]-[6-(5- cyclopropyl-4H-1,2,4-triazol-3-yl)- 2-azaspiro[3.3]heptan-2- yl]methanone | 544.2 [M + H]⁺ |
| 416 | 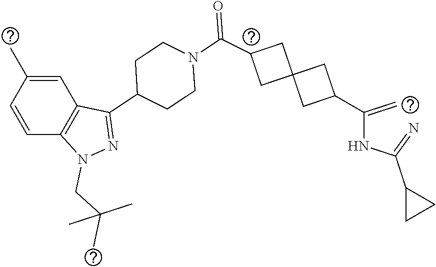 [SMILES: C=C(NC(=N)C1CC1)C1CC2(C1)CC(C(=O)N1CCC(c3nn(CC(C)(C)C)c4ccc(C)cc34)CC1)C2] | [4-[5-chloro-1-(2-hydroxy-2- methyl-propyl)indazol-3- yl]piperidino]-[6-(5-cyclopropyl- 4H-1,2,4-triazol-3-yl)-2- azaspiro[3.3]heptan-2- yl]methanone | 536.3 [M − H]⁻ |
| 429 | 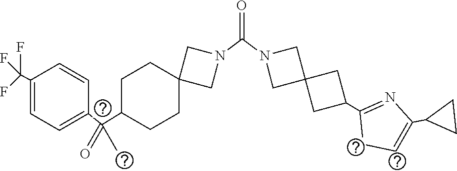 | [6-(3-cyclopropyl-1H-1,2,4-triazol- 5-yl)-2-azaspiro[3.3]heptan-2-yl]- [7-[4- (trifluoromethyl)phenyl] sulfonimidoyl]-2- azaspiro[3.5]nonan-2- yl]methanone | 563.2 [M + H]⁺ |
| 449 | 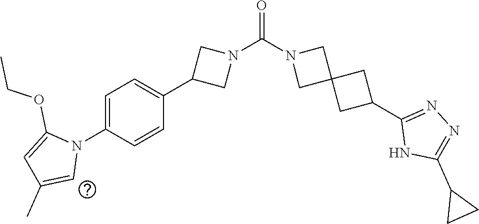 [SMILES: CCOc1cc(C)cn1-c1ccc(C2CN(C(=O)N3CC4(CC(c5nnc(C6CC6)[nH]5)C4)C3)C2)cc1] | [6-(5-cyclopropyl-4H-1,2,4-triazol- 3-yl)-2-azaspiro[3.3]heptan-2-yl]- [3-[4-(5-ethoxy-3-methyl-pyrazol- 1-yl)phenyl]azetidin-1- yl]methanone | 486.5 [M − H]⁻ |
| 456 | 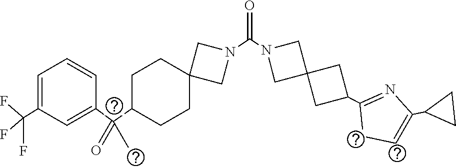 | [6-(3-cyclopropyl-1H-1,2,4-triazol- 5-yl)-2-azaspiro[3.3]heptan-2-yl]- [7-[3- (trifluoromethyl)phenyl] sulfonimidoyl]-2- azaspiro[3.5]nonan-2- yl]methanone | 563.2 [M + H]⁺ |
| 500 | 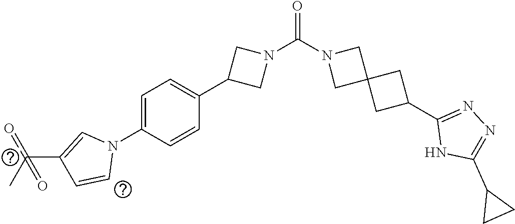 | [6-(5-cyclopropyl-4H-1,2,4-triazol- 3-yl)-2-azaspiro[3.3]heptan-2-yl]- [3-[4-(4-mesylpyrazol-1- yl)phenyl]azetidin-1-yl]methanone | 508.2 [M + H]⁺ |
| 516 | 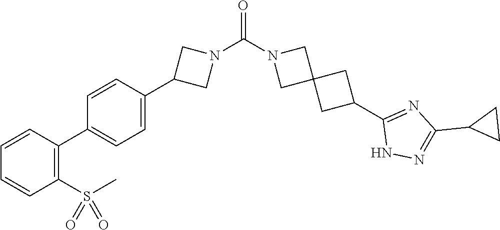 [SMILES: CS(=O)(=O)c1ccccc1-c1ccc(C2CN(C(=O)N3CC4(CC(c5nc(C6CC6)n[nH]5)C4)C3)C2)cc1] | [6-(3-cyclopropyl-1H-1,2,4-triazol- 5-yl)-2-azaspiro[3.3]heptan-2-yl]- [3-[4-(2- mesylphenyl)phenyl]azetidin-1- yl]methanone | 518.2 [M + H]⁺ |
| 538 | 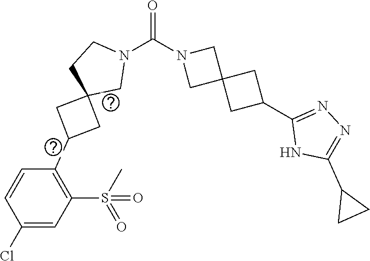 | [2-(4-chloro-2-mesyl-phenyl)-6- azaspiro[3.4]octan-6-yl]-[6-(5- cyclopropyl-4H-1,2,4-triazol-3-yl)- 2-azaspiro[3.3]heptan-2- yl]methanone | 530.2 [M + H]⁺ |
| 559 | 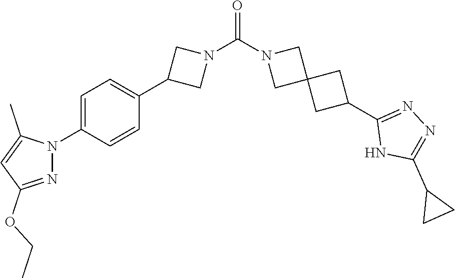 [SMILES: CCOc1cc(C)n(-c2ccc(C3CN(C(=O)N4CC5(CC(c6nnc(C7CC7)[nH]6)C5)C4)C3)cc2)n1] | [6-(5-cyclopropyl-4H-1,2,4-triazol- 3-yl)-2-azaspiro[3.3]heptan-2-yl]- [3-[4-(3-ethoxy-5-methyl-pyrazol- 1-yl)phenyl]azetidin-1- yl]methanone | 486.5 [M − H]⁻ |
| 576 | 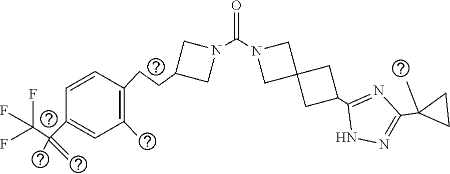 | [3-[2-fluoro-4- (trifluoromethylsulfonimidoyl) benzyl]oxyazetidin-1-yl]-[6-[3-(1- hydroxycyclopropyl)-1H-1,2,4- triazol-5-yl]-2-azaspiro[3.3]heptan- 2-yl]methanone | 559.2 [M + H]⁺ |
| 577 | 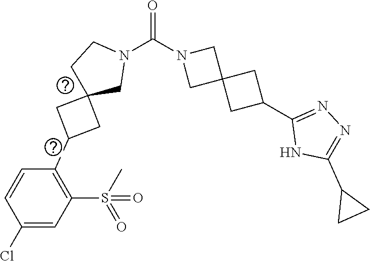 | [2-(4-chloro-2-mesyl-phenyl)-6- azaspiro[3.4]octan-6-yl]-[6-(5- cyclopropyl-4H-1,2,4-triazol-3-yl)- 2-azaspiro[3.3]heptan-2- yl]methanone | 530.2 [M + H]⁺ |
| 600 | 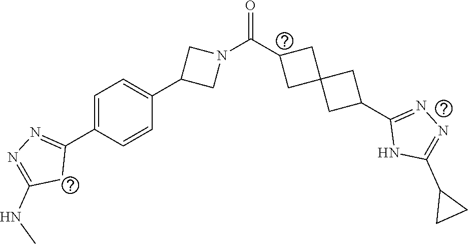 [SMILES: CNC1=NN=C(c2ccc(C3CN(C(=O)C4CC5(C4)CC(c4nnc(C6CC6)[nH]4)C5)C3)cc2)C1] | [6-(5-cyclopropyl-4H-1,2,4-triazol- 3-yl)-2-azaspiro[3.3]heptan-2-yl]- [3-[4-[5-(methylamino)-1,3,4- thiadiazol-2-yl]phenyl]azetidin-1- yl]methanone | 475.5 [M − H]⁻ |
| 632 | 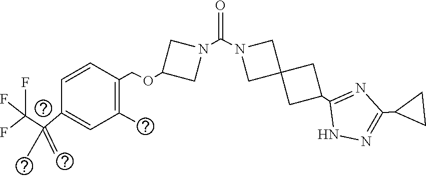 | [6-(3-cyclopropyl-1H-1,2,4-triazol- 5-yl)-2-azaspiro[3,3]heptan-2-yl]- [3-[2-fluoro-4- (trifluoromethylsulfonimidoyl)benz yl]oxyazetidin-1-yl]methanone | 543.2 [M + H]⁺ |
| 648 | 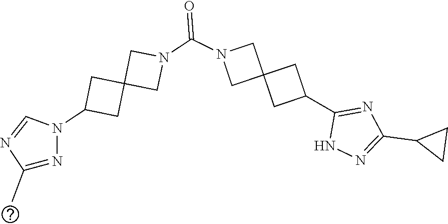 [SMILES: Cc1ncn(C2CC3(C2)CN(C(=O)N2CC4(CC(c5nc(C6CC6)n[nH]5)C4)C2)C3)n1] | [6-(3-cyclopropyl-1H-1,2,4-triazol- 5-yl)-2-azaspiro[3.3]heptan-2-yl]- [6-(3-hydroxy-1,2,4-triazol-1-yl)-2- azaspiro[3.3]heptan-2- yl]methanone | 411.3 [M + H]⁺ |
| 696 | 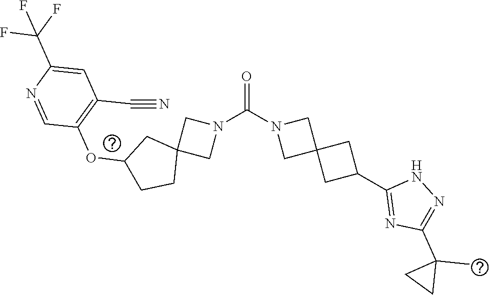 [SMILES: CC1(c2n[nH]c(C3CC4(C3)CN(C(=O)N3CC5(CCC(Oc6cnc(C(F)(F)F)cc6C#N)C5)C3)C4)n2)CC1] | 5-[(6S)-2-[6-[3-(1- hydroxycyclopropyl)-1H-1,2,4- triazol-5-yl]-2- azaspiro[3.3]heptane-2-carbonyl]- 2-azaspiro[3.4]octan-6-yl]oxy]-2- (trifluoromethyl)isonicotinonitrile | 544.3 [M + H]⁺ |
| 735 | 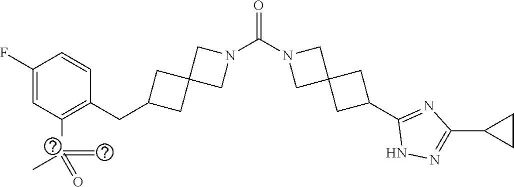 | [6-(3-cyclopropyl-1H-1,2,4-triazol- 5-yl)-2-azaspiro[3.3]heptan-2-yl]- [6-[4-fluoro-2- (methylsulfonimidoyl)benzyl]-2- azaspiro[3.3]heptan-2- yl]methanone | 513.3 [M + H]⁺ |
| 781 | 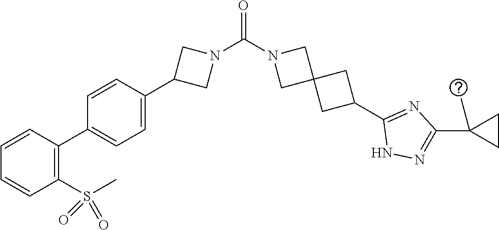 [SMILES: CC1(c2n[nH]c(C3CC4(C3)CN(C(=O)N3CC(c5ccc(-c6ccccc6S(C)(=O)=O)cc5)C3)C4)n2)CC1] | [6-[3-(1-hydroxycyclopropyl)-1H- 1,2,4-triazol-5-yl]-2- azaspiro[3.3]heptan-2-yl]-[3-[4-(2- mesylphenyl)phenyl]azetidin-1- yl]methanone | 534.3 [M + H]⁺ |
| 802 | 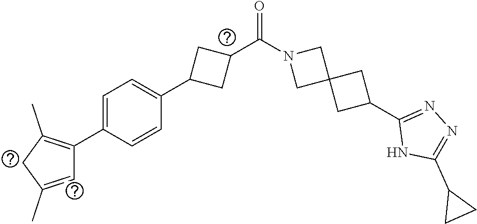 | [6-(5-cyclopropyl-4H-1,2,4-triazol- 3-yl)-2-azaspiro[3.3]heptan-2-yl]- [3-[4-(2,5-dimethylthiazol-4- yl)phenyl]azetidin-1-yl]methanone | 473.4 [M + H]⁺ |
| 806 | 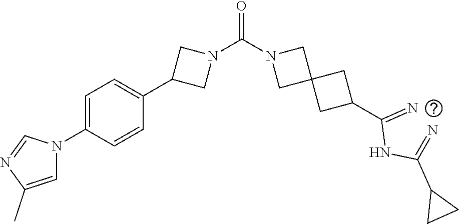 | [6-(5-cyclopropyl-4H-1,2,4-triazol- 3-yl)-2-azaspiro[3.3]heptan-2-yl]- [3-[4-(4-methylimidazol-1- yl)phenyl]azetidin-1-yl]methanone | 222.8 [M + H]²⁺ |
| 836 | 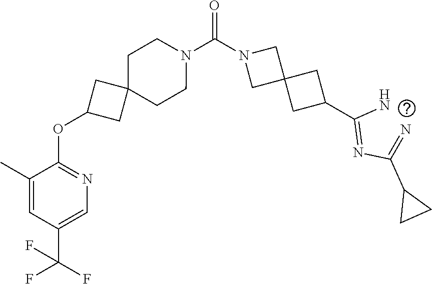 | [6-(3-cyclopropyl-1H-1,2,4-triazol- 5-yl)-2-azaspiro[3.3]heptan-2-yl]- [2-[3-methyl-5-(trifluoromethyl)- 2-pyridyl]oxy]-7- azaspiro[3.5]nonan-7-yl]methanone | 531.4 [M + H]⁺ |
| 840 | 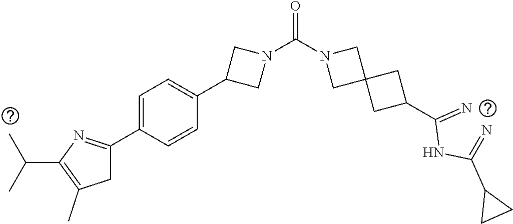 | [6-(5-cyclopropyl-4H-1,2,4-triazol- 3-yl)-2-azaspiro[3.3]heptan-2-yl]- [3-[4-[4-(1-hydroxyethyl)-5- methyl-thiazol-2- yl]phenyl]azetidin-1-yl]methanone | 505.3 [M + H]⁺ |
| 842 | 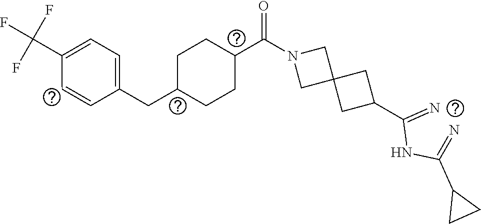 [SMILES: N=C(NC(=N)C1CC2(C1)CN(C(=O)C1CCC(Cc3ccc(C(F)(F)F)cc3)CC1)C2)C1CC1] | [6-(5-cyclopropyl-4H-1,2,4- triazol-3-yl)-2-azaspiro[3.3]heptan- 2-yl]-[4-[6-(trifluoromethyl)-3- pyridyl]methyl]piperazino] methanone | 476.2 [M + H]⁺ |
| 858 | 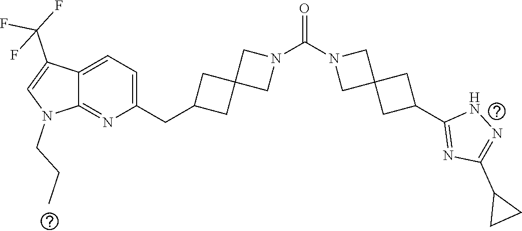 [SMILES: CCCn1cc(C(F)(F)F)c2ccc(CC3CC4(C3)CN(C(=O)N3CC5(CC(c6nc(C7CC7)n[nH]6)C5)C3)C4)nc21] | [6-(3-cyclopropyl-1H-1,2,4-triazol- 5-yl)-2-azaspiro[3.3]heptan-2-yl]- [6-[1-(2-hydroxyethyl)-3- (trifluoromethyl)pyrrolo[2,3- b]pyridin-6-yl]methyl]-2- azaspiro[3.3]heptan-2- yl]methanone | 568.5 [M + H]⁺ |
| 861 | 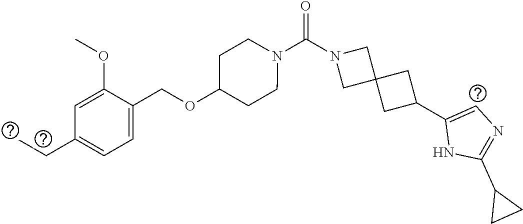 [SMILES: CCc1ccc(COC2CCN(C(=O)N3CC4(CC(c5cnc(C6CC6)[nH]5)C4)C3)CC2)c(OC)c1] | 4-[1-[6-(5-cyclopropyl-4H-1,2,4- triazol-3-yl)-2- azaspiro[3.3]heptane-2-carbonyl]- 4-piperidyl]oxymethyl]-3-methoxy- benzonitrile | 477.3 [M + H]⁺ |
| 864 | 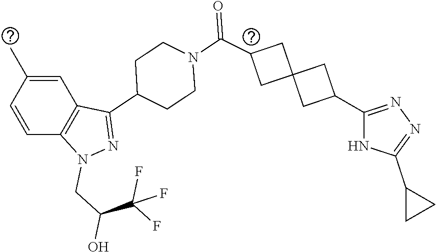 | [4-[5-chloro-1-[(2S)-3,3,3-trifluoro- 2-hydroxy-propyl]indazol-3- yl\|piperidino]-[6-(5-cyclopropyl- 4H-1,2,4-triazol-3-yl)-2- azaspiro[3.3]heptan-2- yl]methanone | 578.3 [M + H]⁺ |
| 865 | 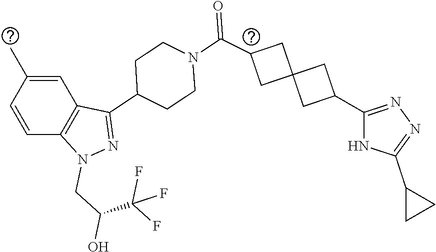 | [4-[5-chloro-1-[(2R)-3,3,3- trifluoro-2-hydroxy-propyl]indazol- 3-yl]piperidino]-[6-(5-cyclopropyl- 4H-1,2,4-triazol-3-yl)-2- azaspiro[3.3]heptan-2- yl]methanone | 578.3 [M + H]⁺ |
| 870 | 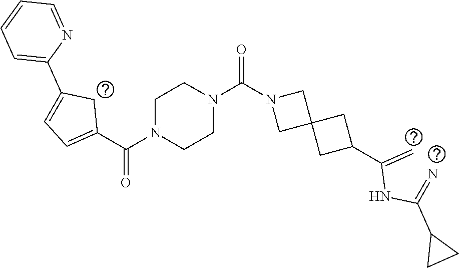 [SMILES: C=C(NC(=N)C1CC1)C1CC2(C1)CN(C(=O)N1CCN(C(=O)C3=CC=C(c4ccccn4)C3)CC1)C2] | [4-[6-(5-cyclopropyl-4H-1,2,4- triazol-3-yl)-2- azaspiro[3.3]heptane-2- carbonyl]piperazino]-[5-(2- pyridyl)-2-thienyl]methanone | 504.2 [M + H]⁺ |
| 871 | 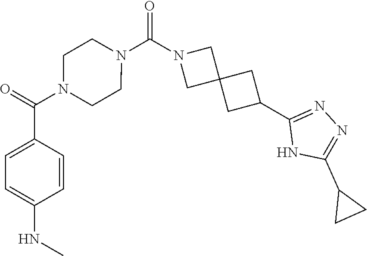 [SMILES: CNc1ccc(C(=O)N2CCN(C(=O)N3CC4(CC(c5nnc(C6CC6)[nH]5)C4)C3)CC2)cc1] | [4-[6-(5-cyclopropyl-4H-1,2,4- triazol-3-yl)-2- azaspiro[3.3]heptane-2- carbonyl]piperazino]-[4- (methylamino)phenyl]methanone | 450.3 [M + H]⁺ |
| 872 | 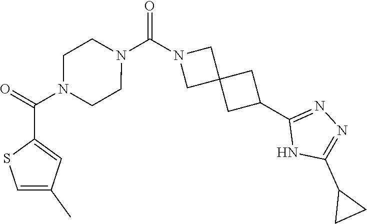 [SMILES: Cc1csc(C(=O)N2CCN(C(=O)N3CC4(CC(c5nnc(C6CC6)[nH]5)C4)C3)CC2)c1] | [4-[6-(5-cyclopropyl-4H-1,2,4- triazol-3-yl)-2- azaspiro[3.3]heptane-2- carbonyl]piperazino]-(4-methyl-2- thienyl)methanone | 441.2 [M + H]⁺ |
| 876 | 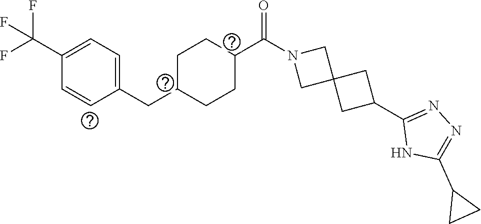 [SMILES: O=C(C1CCC(Cc2ccc(C(F)(F)F)cc2)CC1)N1CC2(CC(c3nnc(C4CC4)[nH]3)C2)C1] | [6-(5-cyclopropyl-4H-1,2,4-triazol- 3-yl)-2-azaspiro[3.3]heptan-2-yl]- [4-[5-(trifluoromethyl)-2- pyridyl]methyl]piperazino] methanone | 474.2 [M − H]⁻ |
| 879 | 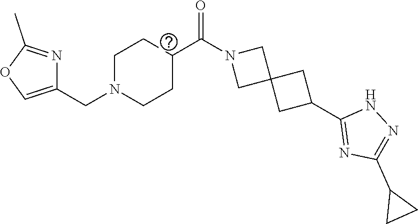 | [6-(3-cyclopropyl-1H-1,2,4-triazol- 5-yl)-2-azaspiro[3.3]heptan-2-yl]- [4-[(2-methyloxazol-4- yl)methyl]piperazino]methanone | 456.3 [M + HC OO]⁻ |
| 880 | 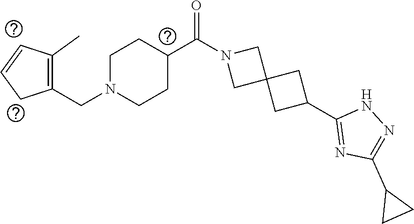 | [6-(3-cyclopropyl-1H-1,2,4-triazol- 5-yl)-2-azaspiro[3.3]heptan-2-yl]- [4-[(4-methylthiazol-5- yl)methyl]piperazino]methanone | 427.3 [M + H]⁺ |
| 881 | 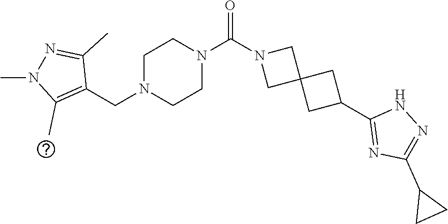 | [4-[(5-chloro-1,3-dimethyl-pyrazol- 4-yl)methyl]piperazino]-[6-(3- cyclopropyl-1H-1,2,4-triazol-5-yl)- 2-azaspiro[3.3]heptan-2- yl]methanone | 503.3 [M + HC OO]⁻ |
| 885 | 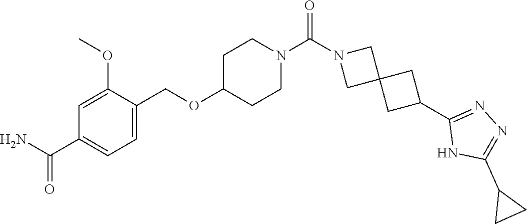 [SMILES: COc1cc(C(N)=O)ccc1COC1CCN(C(=O)N2CC3(CC(c4nnc(C5CC5)[nH]4)C3)C2)CC1] | 4-[[1-[6-(5-cyclopropyl-4H-1,2,4- triazol-3-yl)-2- azaspiro[3.3]heptane-2-carbonyl]- 4-piperidyl]oxymethyl]-3-methoxy- benzamide | 495.3 [M + H]⁺ |
| 891 | 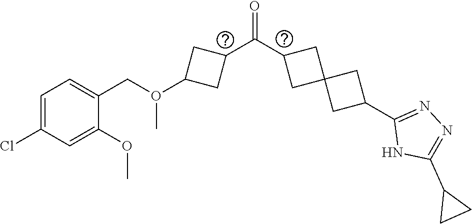 | [3-[(4-chloro-2-methoxy-benzyl)- methyl-amino]azetidin-1-yl]-[6-(5- cyclopropyl-4H-1,2,4-triazol-3-yl)- 2-azaspiro[3.3]heptan-2- yl]methanone | 471.3 [M + H]⁺ |
| 892 | 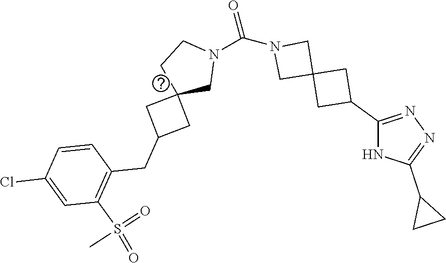 | [2-(4-chloro-2-mesyl-benzyl)-6- azaspiro[3.4]octan-6-yl]-[6-(5- cyclopropyl-4H-1,2,4-triazol-3-yl)- 2-azaspiro[3.3]heptan-2- yl]methanone | 544.2 [M + H]⁺ |
| 893 | 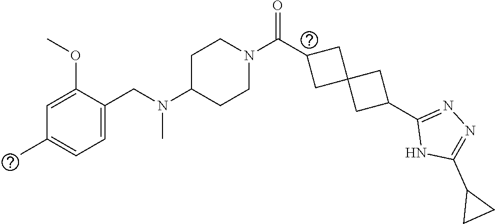 | [4-[(4-chloro-2-methoxy-benzyl)- methyl-amino]piperidino]-[6-(5- cyclopropyl-4H-1,2,4-triazol-3-yl)- 2-azaspiro[3.3]heptan-2- yl]methanone | 499.3 [M + H]⁺ |
| 895 | 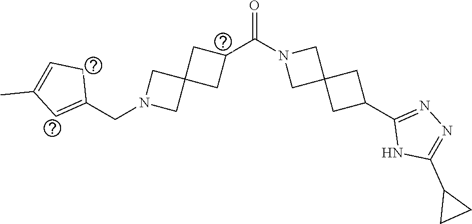 | [6-(5-cyclopropyl-4H-1,2,4-triazol- 3-yl)-2-azaspiro[3.3]heptan-2-yl]- [6-[(4-methylthiazol-2-yl)methyl]- 2,6-diazaspiro[3.3]heptan-2- yl]methanone | 440.2 [M + H]⁺ |
| 899 | 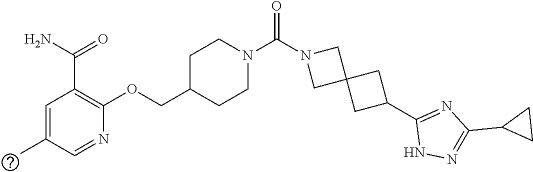 | 5-chloro-2-[1-[6-(3-cyclopropyl- 1H-1,2,4-triazol-5-yl)-2- azaspiro[3.3]heptane-2-carbonyl]- 4-piperidyl]methoxy]nicotinamide | 500.2 [M + H]⁺ |
| 902 | 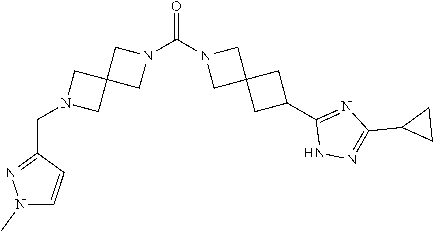 [SMILES: Cn1ccc(CN2CC3(C2)CN(C(=O)N2CC4(CC(c5nc(C6CC6)n[nH]5)C4)C2)C3)n1] | [6-(3-cyclopropyl-1H-1,2,4-triazol- 5-yl)-2-azaspiro[3.3]heptan-2-yl]- [6-[(1-methylpyrazol-3-yl)methyl]- 2,6-diazaspiro[3.3]heptan-2- yl]methanone | 423.3 [M + H]⁺ |
| 903 | 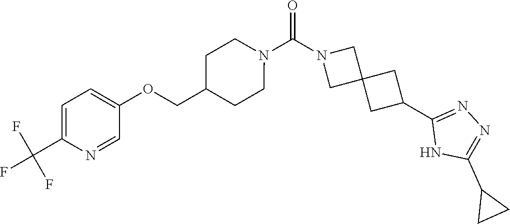 [SMILES: O=C(N1CCC(COc2ccc(C(F)(F)F)nc2)CC1)N1CC2(CC(c3nnc(C4CC4)[nH]3)C2)C1] | [6-(5-cyclopropyl-4H-1,2,4-triazol- 3-yl)-2-azaspiro[3.3]heptan-2-yl]- [4-[6-(trifluoromethyl)-3- pyridyl]oxymethyl]piperidino] methanone | 491.3 [M + H]⁺ |
| 904 | 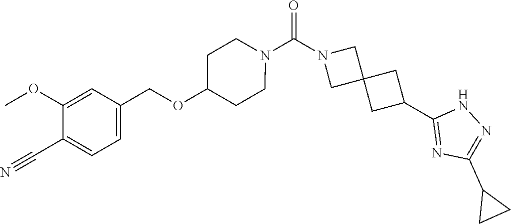 [SMILES: COc1cc(COC2CCN(C(=O)N3CC4(CC(c5nc(C6CC6)n[nH]5)C4)C3)CC2)ccc1C#N] | 4-[1-[6-(3-cyclopropyl-1H-1,2,4- triazol-5-yl)-2- azaspiro[3.3]heptane-2-carbonyl]- 4-piperidyl]oxymethyl]-2-methoxy- benzonitrile | 477.2 [M + H]⁺ |
| 906 | 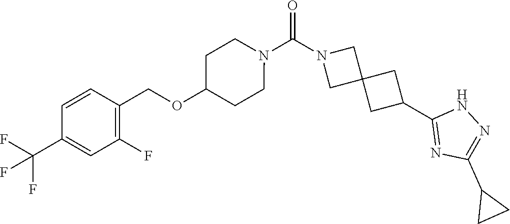 [SMILES: O=C(N1CCC(OCc2ccc(C(F)(F)F)cc2F)CC1)N1CC2(CC(c3nc(C4CC4)n[nH]3)C2)C1] | [6-(3-cyclopropyl-1H-1,2,4-triazol- 5-yl)-2-azaspiro[3.3]heptan-2-yl]- [4-[2-fluoro-4- (trifluoromethyl)benzyl]oxypiperidi no]methanone | 508.2 [M + H]⁺ |
| 907 | 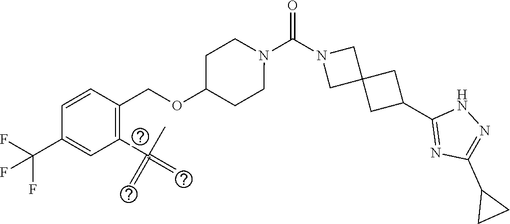 | [6-(3-cyclopropyl-1H-1,2,4-triazol- 5-yl)-2-azaspiro[3.3]heptan-2-yl]- [4-[2-mesyl-4- (trifluoromethyl)benzyl]oxypiperidi no]methanone | 568.3 [M + H]⁺ |
| 908 | 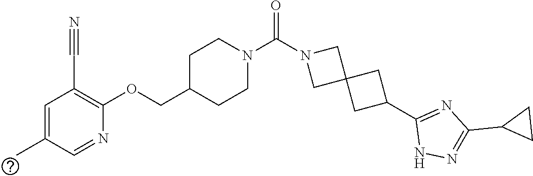 | 5-chloro-2-[1-[6-(3-cyclopropyl- 1H-1,2,4-triazol-5-yl)-2- azaspiro[3.3]heptane-2-carbonyl]- 4-piperidyl]methoxy]nicotinonitrile | 482.2 [M + H]⁺ |
| 910 | 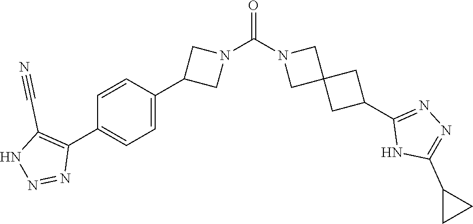 [SMILES: N#Cc1[nH]nnc1-c1ccc(C2CN(C(=O)N3CC4(CC(c5nnc(C6CC6)[nH]5)C4)C3)C2)cc1] | 4-[4-[1-[6-(5-cyclopropyl-4H- 1,2,4-triazol-3-yl)-2- azaspiro[3.3]heptane-2- carbonyl]azetidin-3-yl]phenyl]-1H- triazole-5-carbonitrile | 456.2 [M + H]⁺ |
| 915 | 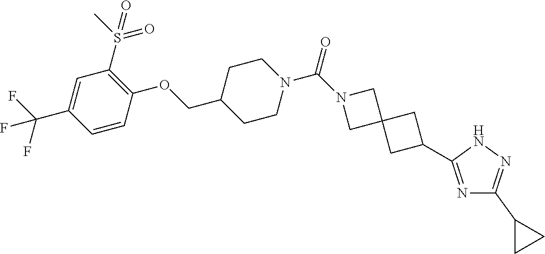 [SMILES: CS(=O)(=O)c1cc(C(F)(F)F)ccc1OCC1CCN(C(=O)N2CC3(CC(c4nc(C5CC5)n[nH]4)C3)C2)CC1] | [6-(3-cyclopropyl-1H-1,2,4-triazol- 5-yl)-2-azaspiro[3.3]heptan-2-yl]- [4-[[2-mesyl-4- (trifluoromethyl)phenoxy]methyl] piperidino]methanone | 568.2 [M + H]⁺ |
| 916 | 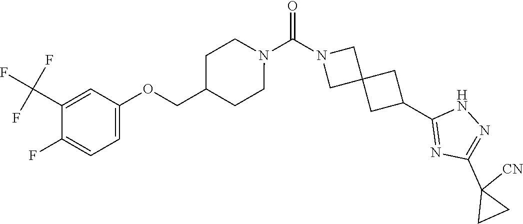 [SMILES: N#CC1(c2n[nH]c(C3CC4(C3)CN(C(=O)N3CCC(COc5ccc(F)c(C(F)(F)F)c5)CC3)C4)n2)CC1] | [4-[[4-fluoro-3- (trifluoromethyl)phenoxy]methyl] piperidino]-[6-[3-(1- hydroxycyclopropyl)-1H-1,2,4- triazol-5-yl]-2-azaspiro[3.3]heptan- 2-yl]methanone | 524.2 [M + H]⁺ |
| 917 | 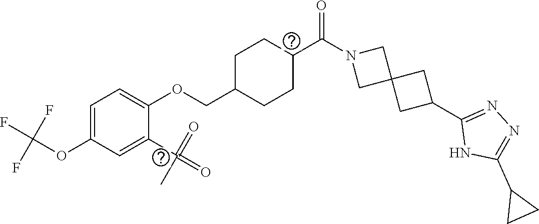 | [6-(5-cyclopropyl-4H-1,2,4-triazol- 3-yl)-2-azaspiro[3.3]heptan-2-yl]- [4-[[2-mesyl-4- (trifluoromethoxy)phenoxy]methyl] piperidino]methanone | 584.2 [M + H]⁺ |
| 918 | 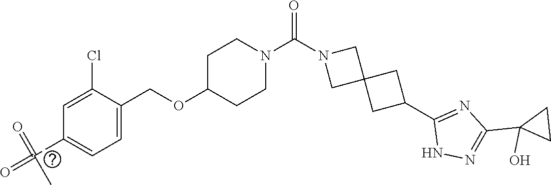 | [4-(2-chloro-4-mesyl- benzyl)oxypiperidino]-[6-[3-(1- hydroxycyclopropyl)-1H-1,2,4- triazol-5-yl]-2-azaspiro[3.3]heptan- 2-yl]methanone | 550.2 [M + H]⁺ |
| 919 | 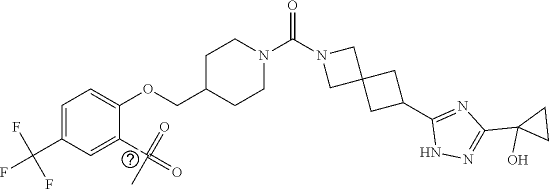 | [6-[3-(1-hydroxycyclopropyl)-1H- 1,2,4-triazol-5-yl]-2- azaspiro[3.3]heptan-2-yl]-[4-[[2- mesyl-4- (trifluoromethyl)phenoxy]methyl] piperidino]methanone | 584.2 [M + H]⁺ |
| 921 | 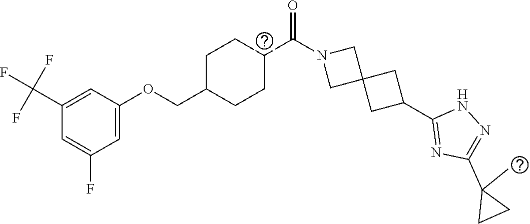 | [4-[[3-fluoro-5- (trifluoromethyl)phenoxy]methyl] piperidino]-[6-[3-(1- hydroxycyclopropyl)-1H-1,2,4- triazol-5-yl]-2-azaspiro[3.3]heptan- 2-yl]methanone | 524.2 [M + H]⁺ |
| 922 | 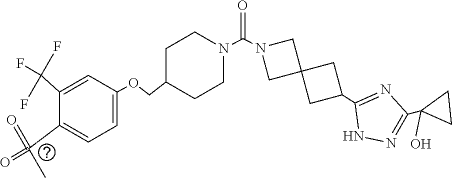 | [6-[3-(1-hydroxycyclopropyl)-1H- 1,2,4-triazol-5-yl]-2- azaspiro[3.3]heptan-2-yl]-[4-[4- mesyl-3- (trifluoromethyl)phenoxy]methyl] piperidino]methanone | 584.2 [M + H]⁺ |
| 923 | 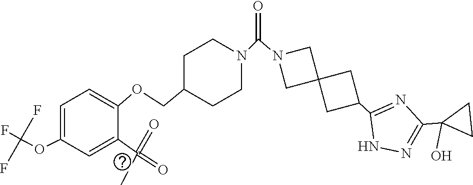 | [6-[3-(1-hydroxycyclopropyl)-1H- 1,2,4-triazol-5-yl]-2- azaspiro[3.3]heptan-2-yl]-[4-[2- mesyl-4- (trifluoromethoxy)phenoxy]methyl] piperidino]methanone | 600.2 [M + H]⁺ |
| 924 | 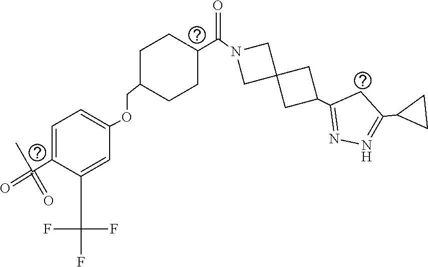 | [6-(5-cyclopropyl-1H-1,2,4-triazol- 3-yl)-2-azaspiro[3.3]heptan-2-yl]- [4-[[4-mesyl-3- (trifluoromethyl)phenoxy]methyl] piperidino]methanone | 568.2 [M + H]⁺ |
| 925 | 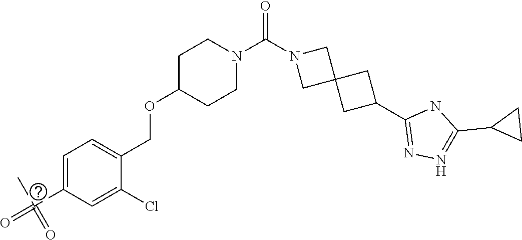 | [4-(2-chloro-4-mesyl- benzyl)oxypiperidino]-[6-(5- cyclopropyl-1H-1,2,4-triazol-3-yl)- 2-azaspiro[3.3]heptan-2- yl]methanone | 534.2 [M + H]⁺ |
| 932 | 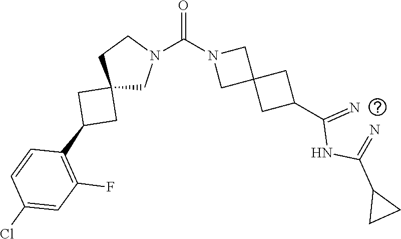 [SMILES: N=C(NC(=N)C1CC2(C1)CN(C(=O)N1CC[C@]3(C1)C[C@H](c1ccc(Cl)cc1F)C3)C2)C1CC1] | [2-(4-chloro-2-fluoro-phenyl)-6- azaspiro[3.4]octan-6-yl]-[6-(5- cyclopropyl-4H-1,2,4-triazol-3-yl)- 2-azaspiro[3.3]heptan-2- ylJmethanone | 470.4 [M + H]⁺ |
| 933 | 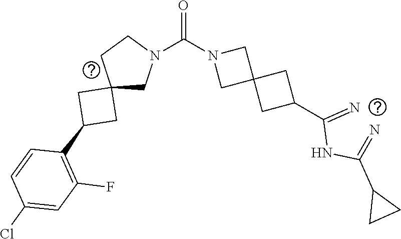 [SMILES: N=C(NC(=N)C1CC2(C1)CN(C(=O)N1CC[C@]3(C1)C[C@@H](c1ccc(Cl)cc1F)C3)C2)C1CC1] | [2-(4-chloro-2-fluoro-phenyl)-6- azaspiro[3.4]octan-6-yl]-[6-(5- cyclopropyl-4H-1,2,4-triazol-3-yl)- 2-azaspiro [3.3]heptan-2- yl]methanone | 470.4 [M + H]⁺ |
| 955 | 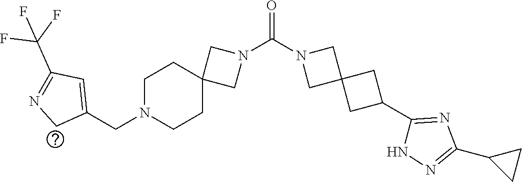 [SMILES: O=C(N1CC2(CCN(CC3=CC(C(F)(F)F)=NC3)CC2)C1)N1CC2(CC(c3nc(C4CC4)n[nH]3)C2)C1] | [6-(3-cyclopropyl-1H-1,2,4-triazol- 5-yl)-2-azaspiro[3.3]heptan-2-yl]- [7-[[3-(trifluoromethyl)isoxazol-5- yl]methyl]-2,7- diazaspiro[3.5]nonan-2- yl]methanone | 506.5 [M + H]⁺ |
| 958 | 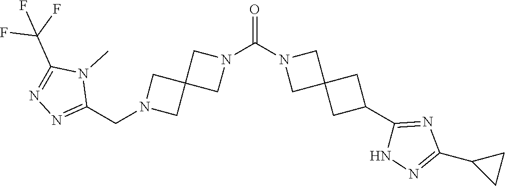 [SMILES: Cn1c(CN2CC3(C2)CN(C(=O)N2CC4(CC(c5nc(C6CC6)n[nH]5)C4)C2)C3)nnc1C(F)(F)F] | [6-(3-cyclopropyl-1H-1,2,4-triazol- 5-yl)-2-azaspiro[3.3]heptan-2-yl]- [6-[[4-methyl-5-(trifluoromethyl)- 1,2,4-triazol-3-yl]methyl]-2,6- diazaspiro[3.3]heptan-2- yl]methanone | 492.5 [M + H]⁺ |
| 979 | 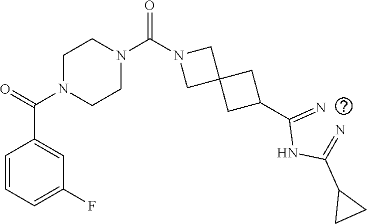 [SMILES: N=C(NC(=N)C1CC2(C1)CN(C(=O)N1CCN(C(=O)c3cccc(F)c3)CC1)C2)C1CC1] | [4-[6-(5-cyclopropyl-4H-1,2,4- triazol-3-yl)-2- azaspiro[3.3]heptane-2- carbonyl]piperazino]-(3- fluorophenyl)methanone | 439.2 [M + H^{]+} |
| 980 | 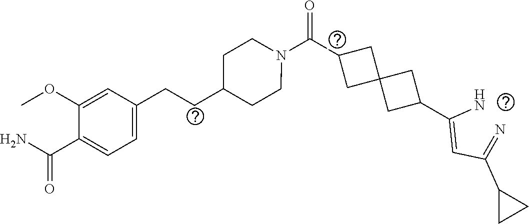 | 4-[[1-[6-(3-cyclopropyl-1H-1,2,4- triazol-5-yl)-2- azaspiro[3.3]heptane-2-carbonyl]- 4-piperidyl]oxymethyl]-2-methoxy- benzamide | 495.3 [M + H]⁺ |

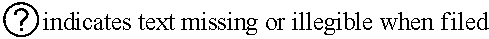

### Synthesis of Building Blocks

### Example A. 1

### 6-(5-cyclopropyl-4H-1,2,4-triazol-3-yl)-2-azaspiro[3.3]heptane; 2,2,2-trifluoroacetic Acid

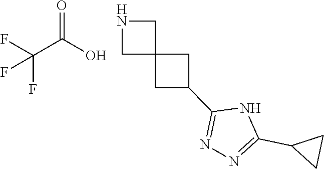

To a solution of tert-butyl 6-(5-cyclopropyl-4H-1,2,4-triazol-3-yl)-2-azaspiro[3.3]heptane-2-carboxylate (150.0 g, 492.79 mmol) in DCM (1.5 L) was added 2,2,2-trifluoroacetic acid (280.94 g, 2464 mmol). The mixture was stirred for 12 h at 25° C., before being evaporated. The residue was diluted with PE/MTBE (200 mL/100 mL) and stirred for 18 h at 25° C. The resulting precipitate was filtered off and collected. The precipitate was diluted with EtOAc and stirred for 2 h at 25° C., and the mixture was filtered off again. The cake was dried, to give the title compound (100.01 g, 63.8% yield) as a white solid. MS (ESI): m/z=205.2 [M+H]⁺

### Step a): Tert-Butyl 6-(5-cyclopropyl-4H-1,2,4-triazol-3 yl)-2-azaspiro[3.3]heptane-2-carboxylate

To a solution of tert-butyl 6-cyano-2-azaspiro[3.3]heptane-2-carboxylate (120.0 g, 539.86 mmol), cyclopropane carbohydrazide (81.08 g, 809.79 mmol) in 1-butanol (1200 mL) was added K₂CO₃ (223.84 g, 1620 mmol), at 23° C. The mixture was heated to 125° C. and stirred for 60 h at this temperature, before being cooled down. The mixture was filtered off, and the residue was evaporated. Purification by FC (SiO₂; PE/EtOAc) gave the title compound (100 g, 60.85% yield) as a yellow solid. MS (ESI): m/z=305.2 [M+H]⁺

### Example A.2

### 1-[5-(2-azaspiro[3.3]heptan-6-yl)-4H-1,2,4-triazol-3 yl]cyclopropanol; 2,2,2-trifluoroacetic Acid

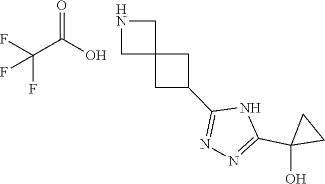

To a solution of 6-[3-(1-hydroxycyclopropyl)-1H-1,2,4-triazol-5-yl]-2-azaspiro[3.3]heptane-2-carboxylic acid tert-butyl ester (2.21 g, 6.9 mmol) in DCM (25 mL) was added TFA (5.31 mL, 68.98 mmol). The mixture was stirred for 18 h at 23° C. before being evaporated, to give the crude title compound (4.59 g, 99.6%). MS (ESI): m/z=221.1 [M+H]⁺

### Step a): Tert-Butyl 6-carbamothioyl-2-azaspiro[3.3]heptane-2-carboxylate

To a solution of tert-butyl 6-cyano-2-azaspiro[3.3]heptane-2-carboxylate (CAS RN: 1374658-94-2, 59.3 g, 266.78 mmol) in DMF (600 mL) was added MgCl₂(25.06 g, 266.78 mmol) at 0° C., followed by dropwise addition of (NH₄)₂S (182.6 mL, 533.56 mmol), at 0° C. The mixture was stirred for 12 h at 25° C., before being diluted with water (2.0 L) and extracted with EtOAc (3×0.5 L). The combined organic layers were washed with brine, dried over Na₂SO₄, filtered, and evaporated, to give the title compound (70.5 g, quant.) as a yellow solid. MS (ESI): m/z=257.2 [M+H]⁺

### Step b): Tert-Butyl 6-(methylsulfanylcarbonimidoyl)-2-azaspiro[3.3]heptane-2-carboxylate; Hydroiodide

To a solution of 6-thiocarbamoyl-2-azaspiro[3.3]heptane-2-carboxylic acid tert-butyl ester (12.87 g, 45.18 mmol) in acetone (240 mL) at 0° C. was added slowly iodomethane (7.7 g, 3.39 mL, 54.22 mmol) over 5 min. The mixture was stirred for 15 min at 0° C., before being allowed to warm up to 23° C. and stirred for another 18 h. The mixture was evaporated, to give the title compound (19.7 g, 99.6% yield) as a white foam. MS (ESI): m/z=271.3 [M+H]⁺

### Step c): 1-hydroxycyclopropanecarbohydrazide

To a solution of 1-hydroxycyclopropanecarboxylic acid methyl ester (25 g, 215.29 mmol) in 1-butanol (300 mL) was added hydrazine 35% aqueous solution (28.96 mL, 322.94 mmol) and the reaction mixture was stirred for 18 h at 80° C. The mixture was evaporated, to give the title compound (24.97 g, 98.0%). MS (ESI): m/z=117.0 [M+H]⁺

### Step d): Tert-Butyl 6-[5-(1-hydroxycyclopropyl)-4H-1,2,4-triazol-3 yl]-2-azaspiro[3.3]heptane-2-carboxylate

To a suspension of 6-[(methylthio)carbonimidoyl]-2-azaspiro[3.3]heptane-2-carboxylic acid tert-butyl ester 0.1:1 hydrogen iodide (18.88 g, 47.4 mmol) in 1-butanol (219.42 mL) was added DIPEA (9.19 g, 12.42 mL, 71.1 mmol) and 1-hydroxycyclopropanecarbohydrazide (6.37 g, 52.14 mmol), at 19° C. The mixture was stirred for 30 min at this temperature, before being heated and stored for 18 h at 90° C. The mixture was cooled down and dissolved in 250 ml of EtOAc. The resulting white precipitate was filtered off. The filtrate was concentrated and diluted with another 200 mL, and the white precipitate was filtered again. The remaining filtrate was diluted with EtOAc, and the organic layer was washed with saturated aqueous NH₄Cl and brine, dried over Na₂SO₄, filtered, and evaporated. The combined crude material was purified by FC (SiO₂; DCM/MeOH), to give the title compound (7.66 g, 56.7% yield) as a white solid. MS (ESI): m/z=319.4 [M−H]⁻

### Example A.3

### 6-(5-cyclobutyl-4H-1,2,4-triazol-3-yl)-2-azaspiro[3.3]heptane; 2,2,2-trifluoroacetic Acid

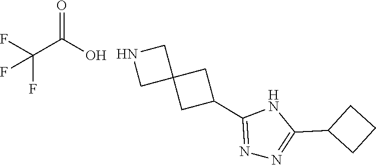

To a solution of 6-(5-cyclobutyl-4H-1,2,4-triazol-3-yl)-2-azaspiro[3.3]heptane-2-carboxylic acid tert-butyl ester (2.30 g, 7.22 mmol) in DCM (18 mL) was added TFA (5.56 mL, 72.23 mmol), at 23° C. The mixture was stirred for 18 h at this temperature before being evaporated, to give the title compound (4.78 g, 99.5% yield). MS (ESI): m/z=219.2 [M+H]⁺

### Step a): Tert-Butyl 6-(5-cyclobutyl-4H-1,2,4-triazol-3-yl)-2-azaspiro[3.3]heptane-2-carboxylate

To a solution of tert-butyl 6-cyano-2-azaspiro[3.3]heptane-2-carboxylate (2.5 g, 11.25 mmol) in 1-butanol (40 mL) were added cyclobutanecarbohydrazide (1.93 g, 16.87 mmol) and potassium carbonate (2.33 g, 16.87 mmol), at 23° C. The mixture was heated to 120° C. and stirred for 72 h at this temperature, before being cooled down and evaporated. The residue was partitioned between EtOAc and saturated aqueous NH₄Cl. The organic layer was collected, and the aqueous phase was extracted with EtOAc (2×). The combined organic layers were washed with brine, dried over Na₂SO₄, filtered, and evaporated. Purification by FC (SiO₂; DCM/MeOH) and SFC gave the title compound (2.49 g, 66.2% yield). MS (ESI): m/z=263.2 [M−tBu+H]⁺

### Example A.4

### 1-[5-(2-azaspiro[3.3]heptan-6-yl)-4H-1,2,4-triazol-3-yl]-2,2,2-trifluoro-ethanol; 2,2,2-trifluoroacetic Acid

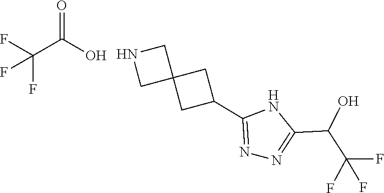

To a solution of 6-[3-(2,2,2-trifluoro-1-hydroxy-ethyl)-1H-1,2,4-triazol-5-yl]-2-azaspiro[3.3]heptane-2-carboxylic acid tert-butyl ester (375 mg, 1.03 mmol) in DCM (5 mL) was added TFA (5.8 mL, 75.26 mmol), at 23° C. The mixture was stirred for another 19 h at this temperature before being evaporated, to give the crude title compound (780 mg, quant.). MS (ESI): m/z=263.1 [M+H]⁺

### Step a): 6-hydroxy-2-azaspiro[3.3]heptane-2-carboxylic Acid Tert-Butyl Ester

To a solution of tert-butyl 6-oxo-2-azaspiro[3.3]heptane-2-carboxylate (CAS RN: 1181816-12-5; 20 g, 94.67 mmol) in MeOH (197.24 mL) was added NaBH₄ (7.16 g, 189.34 mmol), at 0° C. under N₂. The mixture was stirred for 1 h at 0° C., before being evaporated. The residue was dissolved in saturated aqueous NaHCO₃, and the aqueous solution was extracted with DCM. The combined organic layers were dried over MgSO₄, filtered, and evaporated, to give the title compound (19 g, 94% yield) as a white solid. MS (ESI): m/z=158.1 [M+H]⁺

### Step b): Tert-Butyl 6-methylsulfonyloxy-2-azaspiro[3.3]heptane-2-carboxylate

Two batches were conducted in parallel. To a solution of tert-butyl 6-hydroxy-2-azaspiro[3.3]heptane-2-carboxylate (100 g, 468.9 mmol) in DCM (1000 mL) was added TEA (128.0 g, 1172.2 mmol), at 23° C. The reaction mixture was then cooled down to 0° C., and MsCl (81.49 g, 703.3 mmol) was added dropwise. The mixture was stirred for 16 h at 25° C., after which both batches were combined together and poured into water (2 L). The organic layer was separated, washed with brine (2×2000 mL), dried over Na₂SO₄, filtered, and evaporated, to give the title compound (269 g, quant.) as a crude white solid, which was directly used in the next step.

### Step c): Tert-Butyl 6-cyano-2-azaspiro[3.3]heptane-2-carboxylate

Two batches were conducted in parallel. To a solution of tert-butyl 6-methylsulfonyloxy-2-azaspiro[3.3]heptane-2-carboxylate (134.0 g, 459.9 mmol) in DMF (1350 mL) was added NaCN (66.06 g, 1347.9 mmol), at 25° C. The mixture was heated to 100° C. and stirred for 16 h at this temperature, before being poured into ice-cold water (2500 mL, pH>9). The aqueous layer was extracted with EtOAc (2×1 L). The combined organic layers were washed with brine (2×1.5 L), dried over Na2SO4, filtered, and evaporated. Both batches were combined, to give the title compound (125 g, 61.14% yield) as a crude yellow solid, which was directly used in the next step.

### Step d): Tert-Butyl 6-carbamothioyl-2-azaspiro[3.3]heptane-2-carboxylate

To a solution of tert-butyl 6-cyano-2-azaspiro[3.3]heptane-2-carboxylate (59.3 g, 266.78 mmol) in DMF (600 mL) was added MgCl₂ (25.06 g, 266.78 mmol) at 0° C. (NH₄)₂S (182.6 mL, 533.56 mmol) was then added dropwise at 0° C., and the mixture was stirred for 12 h at 25° C. The mixture was diluted with H₂O (2 L), and extracted with EtOAc (3×0.5 L). The combined organic layers were washed with brine (1.5 L), dried over Na₂SO₄, filtered, and evaporated, to give the title compound (70.5 g, quant.) as a yellow solid. MS (ESI): m/z=257.2 [M+H]⁺

### Step e): 6-[3-(2,2,2-trifluoro-1-hydroxy-ethyl)-1H-1,2,4-triazol-5 yl]-2-azaspiro[3.3]heptane-2-carboxylic Acid Tert-Butyl Ester

To a solution of 6-thiocarbamoyl-2-azaspiro[3.3]heptane-2-carboxylic acid tert-butyl ester (1.0 g, 3.71 mmol) in acetone (13 mL) was treated dropwise with iodomethane (278.06 μL, 4.45 mmol), at 0° C. The mixture was stirred for 10 min at this temperature, and another 18 h at 23° C., before being evaporated. The crude residue was added to a solution of 3,3,3-trifluoro-2-hydroxy-propionohydrazide (644.38 mg, 4.08 mmol) and DIPEA (969.94 μL, 5.56 mmol) in 1-butanol (13 mL), and the mixture was stirred for 1 h at 23° C. The mixture was then heated to 100° C. and stirred for 18 h at this temperature, before being cooled down and evaporated. The crude residue was partitioned between EtOAc and saturated aqueous NH₄Cl (pH aqueous phase after extraction 6-7). The organic layer was collected and the aqueous phase was back-extracted with EtOAc. The combined organic layers were dried over Na₂SO₄, filtered, and evaporated. Purification by FC (SiO₂. DCM/MeOH) followed by RP-HPLC gave the title compound (375 mg, 26.5% yield). MS (ESI): m/z=363.2 [M+H]⁺

### Example A.5

### 6-[5-(3,3-difluorocyclobutyl)-4H-1,2,4-triazol-3-yl]-2-azaspiro[3.3]heptane; 2,2,2-trifluoroacetic Acid

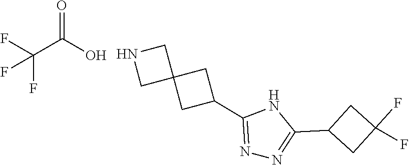

To a solution of 6-[5-(3,3-difluorocyclobutyl)-4H-1,2,4-triazol-3-yl]-2-azaspiro[3.3]heptane-2-carboxylic acid tert-butyl ester (2710 mg, 6.88 mmol) in dichloromethane (20 mL) was added TFA (7.85 g, 5.3 mL, 68.8 mmol) and the reaction mixture was stirred at RT for 18 h. Volatiles were removed in vacuo to give 5010 mg of the crude desired product as colorless viscous oil (purity ˜50%, major contaminant excess of TFA), which was used without further purification. MS (ESI): m/z=255.2 [M-TFA+H]⁺

### Step a) 6-[5-(3,3-difluorocyclobutyl)-4H-1,2,4-triazol-3 yl]-2-azaspiro[3.3]heptane-2-carboxylic Acid Tert-Butyl Ester

To a solution of tert-butyl 6-cyano-2-azaspiro[3.3]heptane-2-carboxylate (CAS: 1374658-94-2) (2.5 g, 11.3 mmol) in 1-butanol (45 mL) was added 3,3-difluorocyclobutanecarbohydrazide (CAS: 1447946-33-9) (2.03 g, 13.5 mmol) and potassium carbonate (1.87 g, 13.5 mmol) and the reaction mixture was stirred at 110° C. for 72 h.

Volatiles were removed in vacuo and the crude residue was then partitioned between 100 mL of ethyl acetate and 75 mL of sat. aq. NH₄Cl solution (pH was adjusted to 6-7 if needed), the organic phase was collected and the aqueous phase was back-extracted twice with 100 mL ethyl acetate. The combined organic phases were dried over sodium sulfate and evaporated down to dryness to yield a crude residue, which was purified by flash chromatography (eluent mixture of heptane and a solution of (EtOAc:EtOH 3:1) (5% to 60%)) to yield the title compound (2.71 g) as a white solid. MS (ESI): m/z=299.1 [M-C₄H₈+H]⁺

In analogy to Example A.5, the following building blocks were made from commercial starting materials:

| | | | | MS, |
|---|---|---|---|---|
| | | | Building | ESI: |
| Ex. | Structure | Systematic Name | Blocks | m/z |
| A.6 | 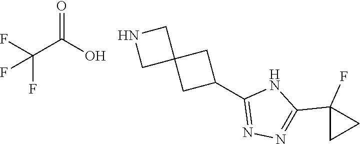 [SMILES: O=C(O)C(F)(F)F ; FC1(c2nnc(C3CC4(CNC4)C3)[nH]2)CC1] | 6-[5-(1- fluorocyclopropyl)-4/I- 1,2,4-triazol-3-yl]-2- azaspiro[3.3]heptane; 2,2,2-trifluoroacetic acid | 1- fluorocyclo- propane-I- carbohydrazide (CAS: 2257473 -93-9) | 319.0 [M − TsOH + H]⁺ |

### Example A.7

### 6-[5-(oxetan-3-yl)-4H-1,2,4-triazol-3-yl]-2-azaspiro[3.3]heptane; Acetic Acid

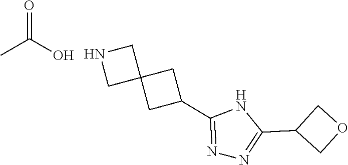

6-[3-(oxetan-3-yl)-1H-1,2,4-triazol-5-yl]-2-azaspiro[3.3]heptane-2-carboxylic acid benzyl ester (1.33 g) was dissolved in MeOH (15 mL) and acetic acid (645 μL), and placed under Argon. 20% Pd(OH)₂/C (wet) (133 mg) was added, and the solution was hydrogenated under at atmosphere of H₂ (3 bar) at 40° C. over 18 h, then cooled, the atmosphere replaced with Argon, and the solution filtered to give a filtrate. The obtained filtrate was concentrated in vacuo to give 1230 mg of the crude desired product as a colorless viscous oil, purity has been roughly estimated to at least 80%, which was used directly without further purification. MS (ESI): m/z=221.2 [M-TFA+H]⁺

### Step a) 6-methylsulfonyloxy-2-azaspiro[3.3]heptane-2-carboxylic Acid Benzyl Ester

To a solution of 6-hydroxy-2-azaspiro[3.3]heptane-2-carboxylic acid benzyl ester (CAS: 1363383-32-7) (18 g, 72.8 mmol) in DCM (290 mL) cooled down to 0° C. was added triethylamine (14.7 g, 20.3 mL, 146 mmol) and methanesulfonyl chloride (9.17 g, 6.2 mL, 80.1 mmol) was added dropwise and the mixture was then stirred at 0° C. for 20 min and at room temperature for 17 h. The mixture was diluted with DCM (200 mL) and washed with water (300 mL). The organic phase was washed with brine (250 mL) dried over Na₂SO₄ and evaporated to dryness. The crude desired product was obtained as a yellow solid (23.9 g) which was used without further purification. MS (ESI): m/z=326.1 [M+H]⁺

### Step b) 6-cyano-2-azaspiro[3.3]heptane-2-carboxylic acid benzyl ester

To a solution of 6-methylsulfonyloxy-2-azaspiro[3.3]heptane-2-carboxylic acid benzyl ester (23300 mg, 71.6 mmol) in N,N-dimethylformamide (250 mL) was added NaCN (7.02 g, 143 mmol). The mixture was heated to 100° C. for 18 h. Volatiles were removed in vacuo and the crude residue was partitioned between ethyl acetate and water. The organic phase was collected and the aqueous phase was back-extracted twice with ethyl acetate. The combined organic phases were dried over sodium sulfate and evaporated down to dryness. The crude material was purified by column chromatography using heptane/ethyl acetate (5-60%) as eluent to give the title compound (13.8 g) as a yellow oil. MS (ESI): m/z=257.3 [M+H]⁺

### Step c) 6-thiocarbamoyl-2-azaspiro[3.3]heptane-2-carboxylic Acid Benzyl Ester

To a solution of 6-cyano-2-azaspiro[3.3]heptane-2-carboxylic acid benzyl ester (10 g, 38.2 mmol) in methanol (174 mL) was added ammonium sulfide (14.8 g, 14.8 mL, 95.6 mmol) and the reaction mixture was stirred at 50° C. for 18 h. Addition of ammonium sulfide (8.88 g, 8.88 mL, 57.4 mmol) and the reaction mixture was again stirred at 50° C. for 18 h. Volatiles were removed in vacuo and the crude residue was partitioned between 250 mL ethyl acetate and 100 mL aq. sol. HCl 1 M. The organic phase was collected and washed with 100 mL brine. The organic phase was dried over sodium sulfate and evaporated down to dryness to yield 11350 mg of the crude title compound as a yellow viscous oil and which was used without further purification. MS (ESI): m/z=291.2 [M+H]⁺

### Step d) 6-[(methylthio)carbonimidoyl]-2-azaspiro[3.3]heptane-2-carboxylic Acid Benzyl ester 1:1 hydrogen iodide

To a solution of 6-thiocarbamoyl-2-azaspiro[3.3]heptane-2-carboxylic acid benzyl ester (2500 mg, 8.61 mmol,) in tetrahydrofuran, extra dry (40 mL) under an inert atmosphere was added iodomethane (1.83 g, 808 μL, 12.9 mmol) and the reaction mixture was stirred at RT for 18 h. Volatiles were removed in vacuo to give 3750 mg of the crude title compound as a light yellow solid which was used without further purification. MS (ESI): m/z=305.1 [M+H]⁺ (Note: after evaporation of THF, the crude residue was sonicated in Ether to obtain a solid after re-evaporation of volatiles)

### Step e) 6-[3-(oxetan-3 yl)-1H-1,2,4-triazol-5 yl]-2-azaspiro[3.3]heptane-2-carboxylic Acid Benzyl Ester

To a round-bottom flask containing 6-[(methylthio)carbonimidoyl]-2-azaspiro[3.3]heptane-2-carboxylic acid benzyl ester 1:1 hydrogen iodide (3400 mg, 7.86 mmol) under an inert atmosphere was added a solution of DIPEA (1.12 g, 1.51 mL, 8.65 mmol) and oxetane-3-carbohydrazide (CAS: 1781585-25-8) (1.19 g, 10.2 mmol) in 1-butanol (40 mL)/methanol (5 mL) (Note: methanol was needed to help the dissolution of the hydrazide which does not seem to be very soluble in 1-butanol) and the reaction mixture was then stirred at RT for 90 min. The reaction mixture was then stirred at 80° C. for 18 h. Volatiles were removed in vacuo, the crude residue was partitioned between ethyl acetate and saturated aqueous solution NH₄Cl, the aqueous phase was collected and the aqueous phase was back-extracted with ethyl acetate. The combined organic phases were dried over sodium sulfate and evaporated down to dryness to give 4.86 g of a residue which was purified by flash chromatography (eluent mixture of heptane and a solution (EtOAc:EtOH 3:1) (5% to 70%)) to give the title compound (809 mg) as a colorless gum. MS (ESI): m/z=355.2 [M+H]⁺

### Example A.8

### 6-[5-(trifluoromethyl)-4H-1,2,4-triazol-3-yl]-2-azaspiro[3.3]heptane; 2,2,2-trifluoroacetic Acid

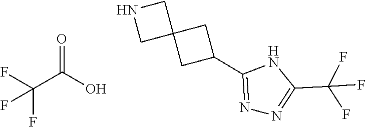 [SMILES: O=C(O)C(F)(F)F ; FC(F)(F)c1nnc(C2CC3(CNC3)C2)[nH]1]

To a solution of 6-[3-(trifluoromethyl)-1H-1,2,4-triazol-5-yl]-2-azaspiro[3.3]heptane-2-carboxylic acid tert-butyl ester (175 mg, 0.448 mmol) in dichloromethane (2 mL) was added TFA (510 mg, 345 μL, 4.48 mmol) after which the reaction mixture was stirred at RT for 4 h. Volatiles were removed in vacuo to give 280 mg of the crude title compound as a viscous orange oil (purity ˜55%, major contaminant excess of TFA) which was used without further purification. MS (ESI): m/z=233.2 [M-TFA+H]⁺

### Step a) 6-thiocarbamoyl-2-azaspiro[3.3]heptane-2-carboxylic Acid Tert-Bury Ester

To a solution of 6-cyano-2-azaspiro[3.3]heptane-2-carboxylic acid tert-butyl ester (CAS: 1374658-94-2) (9100 mg, 40.1 mmol) in methanol (182 mL) was added ammonium sulfide (15.5 g, 15.5 mL, 100 mmol) and the reaction mixture was stirred at 50° C. for 18 h. Volatiles were removed in vacuo, the crude residue was partitioned between 250 mL ethyl acetate and 150 mL of a 1 M HCL aqueous solution, the organic phase was collected and the aqueous phase was back-extracted with 250 mL ethyl acetate. The combined organic phase were dried over sodium sulfate and evaporated down to dryness to give 8210 mg of the crude title compound as an off-white solid which was used without further purification. MS (ESI): m/z=257.2 [M+H]⁺

### Step b) 6-[(methylthio)carbonimidoyl]-2-azaspiro[3.3]heptane-2-carboxylic Acid Tert-Butyl Ester. 1:1 Hydrogen Iodide

To a solution of 6-thiocarbamoyl-2-azaspiro[3.3]heptane-2-carboxylic acid tert-butyl ester (10500 mg, 36.9 mmol) in acetone (156 mL) was added iodomethane (6.28 g, 2.77 mL, 44.2 mmol) and the reaction mixture was stirred at RT for 18 h. Volatiles were removed in vacuo to give 14.8 g of the crude desired product as a light yellow solid which was used without further purification. MS (ESI): m/z=271.3 [M+H]⁺

### Step c) 6-[3-(trifluoromethyl)-1H-1,2,4-triazol-5 yl]-2-azaspiro[3.3]heptane-2-carboxylic Acid Tert-Butyl Ester

To a suspension of 2,2,2-trifluoroacetohydrazide (CAS: 1538-08-5) (530 mg, 4.14 mmol) in 1-butanol (22.5 mL) was added DIPEA (584 mg, 789 μL, 4.52 mmol) and 6-[(methylthio)carbonimidoyl]-2-azaspiro[3.3]heptane-2-carboxylic acid tert-butyl ester.1:1 hydrogen iodide (1500 mg, 3.77 mmol) after which the reaction mixture was stirred at RT for 2 h and at 80° C. for 18 h. Volatiles were removed in vacuo, the crude residue was dissolved in 30 mL ethyl acetate and poured into a separating funnel containing 20 mL 1N HCl aqueous solution for extraction. The organic phase was collected and the aqueous phase was back-extracted with 30 mL ethyl acetate. The combined organic phases were dried over sodium sulfate and evaporated down to dryness. The residue was purified by flash chromatography (eluent mixture of heptane and a solution (EtOAc:EtOH 3:1) (10% to 80%)) to yield the title compound (175 mg) as a yellow solid. MS (ESI): m/z=333.1 [M−H]⁻

### Example A.9

### N-[1-[5-(2-azaspiro[3.3]heptan-6-yl)-1H-1,2,4-triazol-3-yl]cyclopropyl]carbamic Acid Tert-Butyl Ester; Acetic Acid

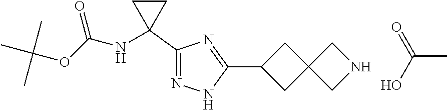

6-[3-[1-(tert-butoxycarbonylamino)cyclopropyl]-1H-1,2,4-triazol-5-yl]-2-azaspiro[3.3]heptane-2-carboxylic acid benzyl ester (4.18 g) was dissolved in MeOH (30 mL) and acetic acid (1.58 mL), and placed under Argon. 20% Pd(OH)₂/C (wet) (418 mg) was added, and the solution was hydrogenated under at atmosphere of H₂ (3 bar) at 40° C. over 18 h, then cooled, the atmosphere replaced with Argon, and the solution filtered to give a filtrate. The obtained filtrate was concentrated in vacuo to give 3480 mg of the crude desired product as a colorless viscous oil, purity has been roughly estimated to at least 80%, which was used directly without further purification. MS (ESI): m/z=320.2 [M−TFA+H]⁺

### Step a) N (1-carbazoylcyclopropyl)carbamic Acid Tert-Butyl Ester

To a solution of 1-(tert-butoxycarbonylamino)cyclopropanecarboxylic acid methyl ester (CAS: 66494-26-6) (3000 mg, 13.9 mmol) in methanol (30 mL) was added hydrazine 35% aqueous solution (2.55 g, 2.5 mL, 27.9 mmol) and the reaction mixture was stirred at 50° C. for 2 days, giving partial conversion. The reaction was then stirred at 70° C. in a sealed vial for 18 h. Volatiles were removed in vacuo and the crude residue was dissolved in 40 mL 1-butanol followed by addition of hydrazine 35% aqueous solution (2.55 g, 2.5 mL, 27.9 mmol). The reaction vial was sealed and stirred at 100° C. for 18 h. A further addition of hydrazine 35% aqueous solution (2.55 g, 2.5 mL, 27.9 mmol) was made, and the reaction mixture was again stirred at 100° C. for 18 h. Volatiles were removed in vacuo to yield the crude title compound (3220 mg) of the crude title compound which was used without further purification. MS (ESI): m/z=160.0 [M-C₄H₈+H]⁺

### Step b) 6-[3-[1-(tert-butoxycarbonylamino)cyclopropyl]-1H-1,2,4-triazol-5-yl]-2-azaspiro[3.3]heptane-2-carboxylic Acid Benzyl Ester

To a round-bottom flask containing 6-[(methylthio)carbonimidoyl]-2-azaspiro[3.3]heptane-2-carboxylic acid benzyl ester 1:1 hydrogen iodide (A.7, Step a) (5190 mg, 10.8 mmol) was added a solution of N-(1-carbazoylcyclopropyl)carbamic acid tert-butyl ester (2.44 g, 11.3 mmol) and DIPEA (2.09 g, 2.83 mL, 16.2 mmol) in 1-butanol (45 mL) and the reaction mixture was stirred at RT under an inert atmosphere for 2 h. The reaction mixture was then stirred at 90° C. for 18 h. Volatiles were removed in vacuo, the crude residue was partitioned between ethyl acetate and sat. aq. NH₄Cl solution and the organic phase was collected. The aqueous phase was back-extracted with ethyl acetate and the combined organic phases were dried over sodium sulfate and evaporated down to dryness to a residue which was purified by flash chromatography (eluent mixture of heptane and a solution (EtOAc:EtOH 3:1) (10% to 70%)) to give the title compound (3570 g) as a white solid. MS (ESI): m/z=454.3 [M+H]⁺

### Example A.10

### 6-(4H-1,2,4-triazol-3-yl)-2-azaspiro[3.3]heptane; 2,2,2-trifluoroacetic Acid

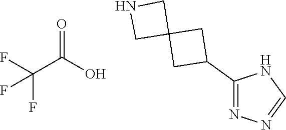 [SMILES: O=C(O)C(F)(F)F ; c1nnc(C2CC3(CNC3)C2)[nH]1]

To a solution of 6-(1H-1,2,4-triazol-5-yl)-2-azaspiro[3.3]heptane-2-carboxylic acid tert-butyl ester (1000 mg, 3.78 mmol) in dichloromethane (15 mL) was added TFA (4.31 g, 2.91 mL, 37.8 mmol) and the reaction mixture was stirred at RT for 18 h. Volatiles were removed in vacuo to give the crude title compound (2100 mg) which was used without further purification. Purity roughly 50% with TFA as major contaminant. MS (ESI): m/z=165.1 [M-TFA+H]⁺

### Step a) 6-(1H-1,2,4-triazol-5-yl)-2-azaspiro[3.3]heptane-2-carboxylic Acid Tert-Butyl Ester

A solution of 6-carbamoyl-2-azaspiro[3.3]heptane-2-carboxylic acid tert-butyl ester (CAS: 1831097-50-7) (4000 mg, 16.7 mmol) in DMF-DMA (39.7 g, 44.6 mL, 333 mmol) was stirred at 90° C. for 2.5 h. Volatiles were removed in vacuo, the obtained crude residue was dissolved in 1,4-dioxane (30 mL) followed by addition of hydrazine (3.05 g, 2.99 mL, 33.3 mmol) and acetic acid (2.0 g, 1.91 mL, 33.3 mmol) after which the reaction mixture was stirred at 90° C. for 18 h. The reaction mixture was poured into a separating funnel containing ethyl acetate and sat. aq. NH₄Cl solution. After the extraction, the organic phase was collected and the aqueous phase was back-extracted with ethyl acetate. The combined organic phases were dried over sodium sulfate and evaporated down to dryness to yield the crude title compound (4.47 g) which was used without further purification. MS (ES): m/z=265.3 [M+H]⁺

### Example B.1

### [3-(2-azaspiro[3.3]heptan-6-ylmethyl)phenyl]-imino-oxo-(trifluoromethyl)-λ⁶-sulfane; 4-methylbenzenesulfonic Acid

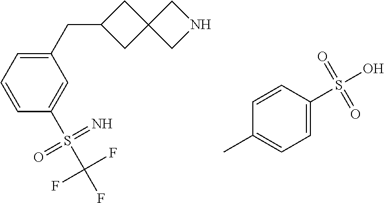 [SMILES: N=S(=O)(c1cccc(CC2CC3(CNC3)C2)c1)C(F)(F)F ; Cc1ccc(S(=O)(=O)O)cc1]

A mixture of p-toluenesulfonic acid (1.18 g, 6.83 mmol) and Tertt-butyl 6-[[3-(trifluoromethylsulfonimidoyl)phenyl]methyl]-2-azaspiro[3.3]heptane-2-carboxylate (1.3 g, 3.11 mmol) in ethyl acetate (30 mL) was stirred at 40° C. for 24 h. After the completion of the reaction, the reaction mixture was concentrated and purified by HPLC to afford the title compound (339 mg, 0.690 mmol, 15.6% yield) as brown viscous oil. MS (ESI): m/z=319.0 [M-TsOH+H]⁺

### Step a) Tert-Butyl 6-[(4,4,5,5-tetramethyl-1,3,2-dioxaborolan-2 yl)methylene]-2-azaspiro[3.3]heptane-2-carboxylate

A mixture of 2,2,6,6-tetramethylpiperidine (95.9 mL, 568 mmol) in THE (750 mL) was cooled to −30° C. under a N₂ atmosphere. n-BuLi (227 mL, 568 mmol) was added dropwise, and the reaction mixture was stirred at the same temperature for 30 min. Next, the reaction was cooled to −60° C., and a solution of 4,4,5,5-tetramethyl-2-[(4,4,5,5-tetramethyl-1,3,2-dioxaborolan-2-yl)methyl]-1,3,2-dioxaborolane (136 g, 506 mmol) in THE (750 mL) was added dropwise. After stirring for 30 min, a solution of tert-butyl 6-oxo-2-azaspiro[3.3]heptane-2-carboxylate (100 g, 473 mmol) in THE (300 mL) was added in dropwise at −60° C. The reaction mixture was allowed to slowly warm up to 25° C. and stirred at 25° C. for 12 h. The mixture was added H₂O (80 mL) slowly and then purified together with an additional batch of equal size by silica gel column (PE/EA=1:0 to 3:1 gradient) to give the title compound (220 g, 656 mmol, approx 69% yield per batch) as a white solid which was confirmed by ¹H NMR (400 MHz, CHLOROFORM-d) δ=5.21-5.16 (m, 1H), 3.99-3.89 (m, 4H), 3.13-2.90 (m, 4H), 1.46-1.41 (m, 9H), 1.26-1.20 ppm (m, 13H).

### Step b) Tert-Butyl 6-[[3-(trifluoromethylsulfonimidoyl)phenyl]methylene]-2-azaspiro[3.3]heptane-2-carboxylate

(3-bromophenyl)-imino-oxo-(trifluoromethyl)-λ6-sulfane (2.47 g, 8.59 mmol), tert-butyl 6-[(4,4,5,5-tetramethyl-1,3,2-dioxaborolan-2-yl)methylene]-2-azaspiro[3.3]heptane-2-carboxylate (2.4 g, 7.16 mmol), 1,1′-bis(diphenylphosphino)ferrocene-palladium(II)dichloride dichloromethane complex (1.17 g, 1.43 mmol) and potassium carbonate (1.98 g, 14.3 mmol) were dissolved in 1,4-Dioxane (40 mL) and water (8 mL). The reaction mixture was heated to 120° C. under argon for 16 h. The reaction mixture was concentrated under reduced pressure. The residue was partitioned between ethyl acetate and water. The organic layer was washed with brine. The extract was dried over sodium sulfate, filtered through a thin layer of silica gel and evaporated. The crude product was purified by column chromatography to afford the title compound (1 g, 2.4 mmol, 31.9% yield) as light yellow solid. MS (ESI): m/z=361.0 [M-tBu+H]⁺.

### Step c) Tert-Butyl 6-[[3-(trifluoromethylsulfonimidoyl)phenyl]methyl]-2-azaspiro[3.3]heptane-2-carboxylate

A mixture of tert-butyl 6-[[3-(trifluoromethylsulfonimidoyl)phenyl]methylene]-2-azaspiro[3.3]heptane-2-carboxylate (1.3 g, 3.12 mmol) and palladium on carbon (10%) (0.16 mL, 1.56 mmol) in EtOAc (35 mL) was stirred in an autoclave for 24 h under 30 bar of H₂. Then the reaction mixture was filtered and concentrated to afford the title compound (1.3 g, 3.11 mmol, 96.5% yield) a as grey oil. MS (ESI): m/z=319.0 [M-Boc+H]⁺.

### Example B.4

### 6-[[5-(trifluoromethyl)pyrazin-2-yl]methyl]-2-azaspiro[3.3]heptane; 4-methylbenzenesulfonic Acid

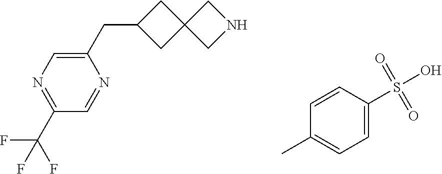 [SMILES: FC(F)(F)c1cnc(CC2CC3(CNC3)C2)cn1 ; Cc1ccc(S(=O)(=O)O)cc1]

A mixture of p-toluenesulfonic acid (10.7 g, 61.9 mmol), andTertt-butyl 6-[[5-(trifluoromethyl)pyrazin-2-yl]methyl]-2-azaspiro[3.3]heptane-2-carboxylate (18.4 g, 51.5 mmol) in EtOAc (180 mL) was stirred at 80° C. for 12 h. The mixture was filtered and the cake was concentrated to give the title compound (7.5 g, 17.5 mmol, 31.1% yield) as grey solid. The filtrate was concentrated and lyophilized to give a further batch of the title compound (1.5.0 g, 34.93 mmol, 67.7% yield) as yellow solid. MS (ESI): m/z=258.2 [M-TsOH+H]⁺. ¹H NMR (400 MHz, DMSO-d₆) δ=9.08 (s, 1H), 8.74 (s, 1H), 8.56-8.35 (m. 2H), 7.48 (d, J=7.9 Hz, 2H), 7.11 (d, J=7.8 Hz, 2H), 3.96 (t, 0.1=6.1 Hz, 2H), 3.87 (t, J=6.2 Hz, 2H), 2.97 (d, J=7.6 Hz, 2H), 2.56 (br d, J=7.9 Hz, 1H), 2.34-2.29 (m, 2H), 2.28 (s, 3H), 2.04-1.94 (m, 2H)

### Step a) Tert-Butyl 6[[5-(trifluoromethyl)pyrazin-2 yl]methylene]-2-azaspiro[3.3]heptane-2-carboxylate

To a solution of 2-bromo-5-(trifluoromethyl)pyrazine WAS: 799557-87-2) (3.39 g, 14.9 mmol) and tert-butyl 6-[(4,4,5,5-tetramethyl-1,3,2-dioxaborolan-2-yl)methylene]-2-azaspiro[3.3]heptane-2-carboxylate (generated in B.1, Step a)) (5.0 g, 14.9 mmol), potassium carbonate (4.12 g, 29.8 mmol) in 1,4-Dioxane (50 mL 1 and water (10 mL) was added 1,1′-bis(diphenylphosphino)ferrocene-palladium(11)dichloride dichloromethane complex (1.22 g, 1.49 mmol), the mixture was degassed with N₂ 3 times and stirred at 80° C. under N₂ atmosphere for 12 h. The mixture was concentrated under reduced pressure to give a residue which was purified by silica gel chromatography (eluent of 0-30% Ethyl acetate/Petroleum ether) to give a residue which was triturated with petroleum ether at 25° C. for 30 min to give the title compound (4.0 g, 76% yield) as a white solid. MS (ESI): m/z=300.0 [M-C₄H₈+H]⁺

### Step b) Tert-Butyl 6-[[5-(trifluoromethyl)pyrazin-2 yl]methyl]-2-azaspiro[3.3]heptane-2-carboxylate

To a solution of tert-butyl 6-[[5-(trifluoromethyl)pyrazin-2-yl]methylene]-2-azaspiro[3.3]heptane-2-carboxylate (5.0 g, 14.1 mmol) in EtOAc (50 mL) was added 10% Pd/C (1.66 g. 1.41 mmol) under N₂. The suspension was degassed under vacuum and purged with H₂ several times. The mixture was stirred under H₂ (15 psi) at 25° C. for 3 h. The reaction mixture was filtered and the filtrate was concentrated to give the title compound (3.0 g, 99% yield) as green solid. MS (ESI): m/z:=302.1 [M-C₄H₈+H]⁺; ¹H NMR (400 MHz, CHLOROFORM-d) δ=8.86 (s, 1H), 8.50 (s, 1H), 3.93 (s, 2H), 3.85 (s, 2H), 2.98 (d, J=7.6 Hz, 2H), 2.63 (spt, J=7.9 Hz; 1H), 2.40-2.25 (m, 2H), 2.02-1.91 (m, 2H), 1.43 (s, 10H)

In analogy to Example B.1 and B.4, the following building blocks were generated using the relevant (hetero)aryl bromide or iodide building block for the Suzuki coupling in Step b. In some cases, alternative salts (e.g. trifluoroacetate, ditosylate, hydrochloride) were also used. To introduce different spiro-ring systems further building block substitutions can be made, for example Example B.25 used tert-butyl 7-oxo-2-azaspiro[3.5]nonane-2-carboxylate (CAS: 1363381-22-9) in place of tert-butyl 6-oxo-2-azaspiro[3.3]heptane-2-carboxylate in Step a), and Example B.26 and B.47 used tert-butyl 6-oxo-2-azaspiro[3.4]octane-2-carboxylate (CAS: 1363382-39-1) in place of tert-butyl 6-oxo-2-azaspiro[3.3]heptane-2-carboxylate in Step a). In some cases the synthesis was planned to include removal of an additional Br group or protecting group in the hydrogenation step, or an additional protecting group in the Boc deprotection step.

| | | | | MS, |
|---|---|---|---|---|
| | | | Building | ESI: |
| Ex. | Structure | Systematic Name | Blocks | m/z |
| B.2 | 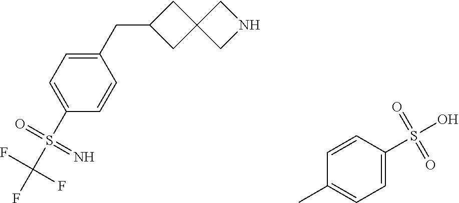 [SMILES: N=S(=O)(c1ccc(CC2CC3(CNC3)C2)cc1)C(F)(F)F ; Cc1ccc(S(=O)(=O)O)cc1] | [4-(2- azaspiro[3.3]heptan-6- ylmethyl)phenyl]- imino-oxo- (trifluoromethyl)-λ⁶- sulfane;4- methylbenzenesulfonic acid | (4- bromophenyl) (imino) (trifluoromethyl)- λ⁶- sulfanone (CAS: 1421133- 03-0) | 319.0 [M − TsOH + H]⁺ |
| B.3 | 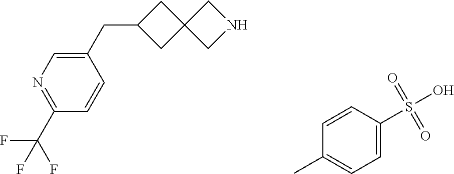 [SMILES: FC(F)(F)c1ccc(CC2CC3(CNC3)C2)cn1 ; Cc1ccc(S(=O)(=O)O)cc1] | 6-[[6-(trifluoromethyl)- 3-pyridyl]methyl]-2- azaspiro[3.3]heptane; 4- methylbenzenesulfonic acid | 5-bromo- 2- (trifluoromethyl) pyridine (CAS: 436799- 32-5) | 257.0 [M − TsOH + H]⁺ |
| B.5 | 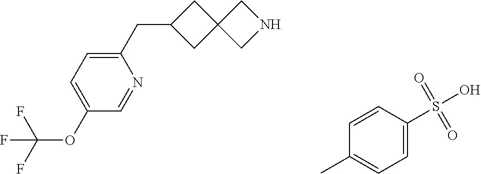 [SMILES: FC(F)(F)Oc1ccc(CC2CC3(CNC3)C2)nc1 ; Cc1ccc(S(=O)(=O)O)cc1] | 6-[6-(trifluoromethyl)- 3-pyridyl]methyl]-2- azaspiro[3.3]heptane; 4- methylbenzenesulfonic acid | 2-bromo- 5- (trifluoro methoxy) pyridine (CAS: 888327- 36-4) | 273.3 [M − TsOH + H]⁺ |
| B.6 | 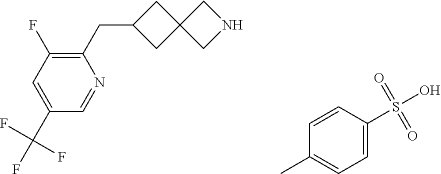 [SMILES: Fc1cc(C(F)(F)F)cnc1CC1CC2(CNC2)C1 ; Cc1ccc(S(=O)(=O)O)cc1] | 6-[[3-fluoro-5- (trifluoromethyl)-2- pyridyl]methyl]-2- azaspiro[3.3]heptane;4- methylbenzenesulfonic acid | 2-bromo- 3-fluoro- 5- (trifluoro methyl) pyridine (CAS: 89402- 29-9) | 447.0 [M + H]⁺ |
| B.7 | 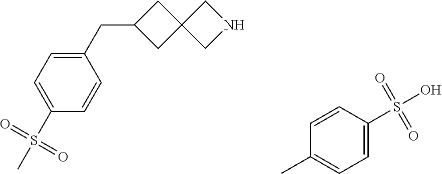 [SMILES: CS(=O)(=O)c1ccc(CC2CC3(CNC3)C2)cc1 ; Cc1ccc(S(=O)(=O)O)cc1] | 6-[(4- methylsulfonylphenyl) methyl]-2- azaspiro[3.3]heptane; 4- methylbenzenesulfonic acid | 4- bromophenyl- methyl sulfone (CAS: 3466-32- 8) | 266.0 [M − TsOH + H]⁺ |
| B.8 | 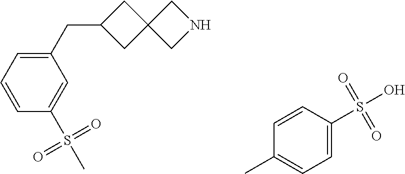 [SMILES: CS(=O)(=O)c1cccc(CC2CC3(CNC3)C2)c1 ; Cc1ccc(S(=O)(=O)O)cc1] | 6-[(3- methylsulfonylphenyl) methyl]-2- azaspiro[3.3]heptane; 4- methylbenzenesulfonic acid | 3- bromophenyl- methylsulfone (CAS: 34896- 80-5) | 265.9 [M − TsOH + H]⁺ |
| B.9 | 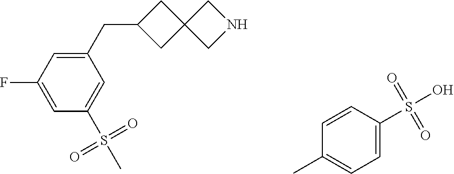 [SMILES: CS(=O)(=O)c1cc(F)cc(CC2CC3(CNC3)C2)c1 ; Cc1ccc(S(=O)(=O)O)cc1] | 6-[(3-fluoro-5- methylsulfonyl- phenyl)methyl]-2- azaspiro[3.3]heptane;4- methylbenzenesulfonic acid | 1-bromo- 3-fluoro-5- methylsulfonyl- benzene (CAS: 1207970-78-2) | 284.0 [M − TsOH + H]⁺ |
| B.10 | 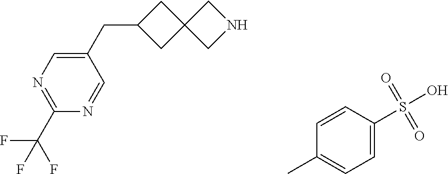 [SMILES: FC(F)(F)c1ncc(CC2CC3(CNC3)C2)cn1 ; Cc1ccc(S(=O)(=O)O)cc1] | 6-[[2- (trifluoromethyl) pyrimidin-5- yl]methyl]-2- azaspiro[3.3]heptane; 4- methylbenzenesulfonic acid | 5-bromo-2- (trifluoromethyl) pyrimidine (CAS: 799557- 86-1) | 258.1 [M − TsOH + H]⁺ |
| B.11 | 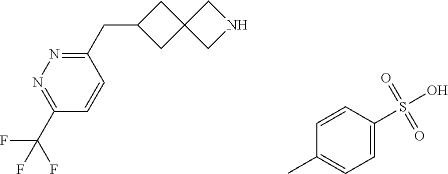 [SMILES: FC(F)(F)c1ccc(CC2CC3(CNC3)C2)nn1 ; Cc1ccc(S(=O)(=O)O)cc1] | 6-[[6- (trifluoromethyl) pyridazin-3- yl]methyl]-2- azaspiro[3.3]heptane; 4- methylbenzenesulfonic acid | 3-bromo-6- (trifluoromethyl) pyridazine (CAS: 174607- 37-5) | 258.4 [M − TsOH + H]⁺ |
| B.12 | 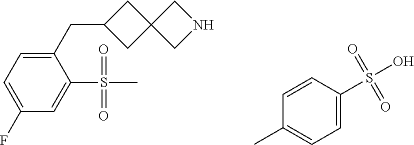 [SMILES: CS(=O)(=O)c1cc(F)ccc1CC1CC2(CNC2)C1 ; Cc1ccc(S(=O)(=O)O)cc1] | 6-[(4-fluoro-2- methylsulfonyl- phenyl)methyl]-2- azaspiro[3.3] heptane; 4- methylbenzenesulfonic acid | 1-bromo- 4-fluoro-2- methanesulfonyl- benzene (CAS: 628692- 10-4) | 284.4 [M − TsOH + H]⁺ |
| B.13 | 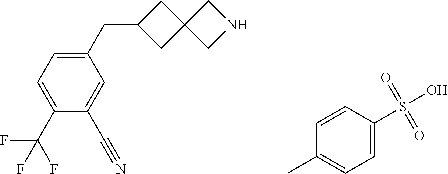 [SMILES: N#Cc1cc(CC2CC3(CNC3)C2)ccc1C(F)(F)F ; Cc1ccc(S(=O)(=O)O)cc1] | 5-(2- azaspiro[3.3]heptan-6- ylmethyl)-2- (trifluoromethyl) benzonitrile; 4- methylbenzenesulfonic acid | 5-bromo-2- (trifluoromethyl) benzonitrile (CAS: 1208076-28-1) | 281.1 [M − TsOH + H]⁺ |
| B.14 | 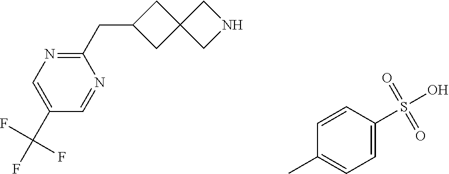 [SMILES: FC(F)(F)c1cnc(CC2CC3(CNC3)C2)nc1 ; Cc1ccc(S(=O)(=O)O)cc1] | 6-[[5- (trifluoromethyl) pyrimidin-2-yl] methyl]-2- azaspiro[3.3]heptane; 4- methylbenzenesulfonic acid | 2-bromo-5- (trifluoromethyl) pyrimidine (CAS: 69034- 09-9) | 257.9 [M − TsOH + H]⁺ |
| B.15 | 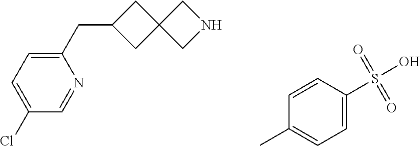 | 6-[(5-chloro-2- pyridyl)methyl]-2- azaspiro[3.3]heptane; 4- methylbenzenesulfonic acid | 2-bromo- 5- chloropyridine | 223.2 [M − TsOH + H]⁺ |
| | *PtOH catalyst used in Step c) | | | |
| B.16 | 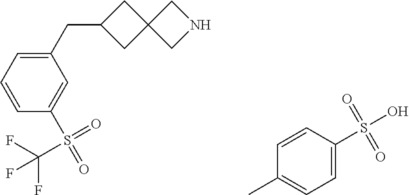 [SMILES: O=S(=O)(c1cccc(CC2CC3(CNC3)C2)c1)C(F)(F)F ; Cc1ccc(S(=O)(=O)O)cc1] | 6-[[3- (trifluoromethylsulfonyl) phenyl]methyl]-2- azaspiro[3.3]heptane; 4- methylbenzenesulfonic acid | 1-bromo-3- ((trifluoromethyl) sulfonyl) benzene (CAS: 2728-70- 3) | 319.0 [M − TsOH + H]⁺ |
| B.17 | 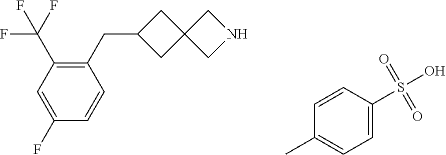 [SMILES: Fc1ccc(CC2CC3(CNC3)C2)c(C(F)(F)F)c1 ; Cc1ccc(S(=O)(=O)O)cc1] | 6-[4-fluoro-2- (trifluoromethyl)phenyl] methyl]-2- azaspiro[3.3]heptane; 4- methylbenzenesulfonic acid | 2-bromo-5- fluoro- benzotrifluoride | 274.0 [M − TsOH + H]⁺ |
| B.18 | 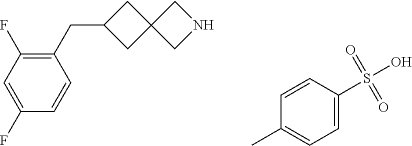 [SMILES: Fc1ccc(CC2CC3(CNC3)C2)c(F)c1 ; Cc1ccc(S(=O)(=O)O)cc1] | 6-[(2,4- difluorophenyl)methyl]- 2-azaspiro[3.3]heptane; 4- methylbenzenesulfonic acid | 1-bromo- 2,4- difluorobenzene | 224.0 [M − TsOH + H]⁺ |
| B.19 | 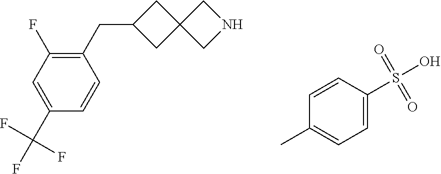 [SMILES: Fc1cc(C(F)(F)F)ccc1CC1CC2(CNC2)C1 ; Cc1ccc(S(=O)(=O)O)cc1] | 6-[[2-fluoro-4- (trifluoromethyl)phenyl] metbyl]-2- azaspiro[3.3]heptane; 4- methylbenzenesulfonic acid | 4-bromo-3- fluoro- benzotrifluoride | 274.0 [M − TsOH + H]⁺ |
| B.20 | 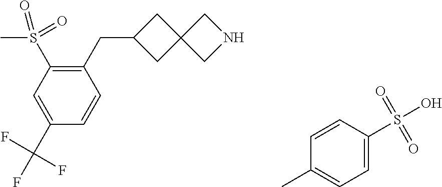 [SMILES: CS(=O)(=O)c1cc(C(F)(F)F)ccc1CC1CC2(CNC2)C1 ; Cc1ccc(S(=O)(=O)O)cc1] | 6-[[2-methylsulfonyl-4- (trifluoromethyl)phenyl] methyl]-2- azaspiro[3.3]heptane; 4- methylbenzenesulfonic acid | 1-bromo-2- methanesulfonyl- 4- (trifluoromethyl) benzene (CAS: 1215205-98-3) | 334.1 [M − TsOH + H]⁺ |
| B.21 | 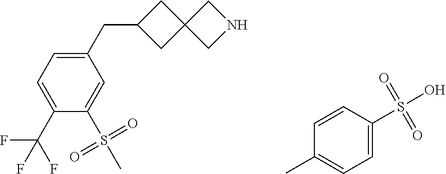 [SMILES: CS(=O)(=O)c1cc(CC2CC3(CNC3)C2)ccc1C(F)(F)F ; Cc1ccc(S(=O)(=O)O)cc1] | 6-[[3-methylsulfonyl-4- (trifluoromethyl)phenyl] methyl]-2- azaspiro[3.3]heptane; 4- methylbenzenesulfonic acid | 4-bromo-2- methylsulfonyl- 1- (trifluoromethyl) benzene (C.1) | 334.0 [M − TsOH + H]⁺ |
| B.22 | 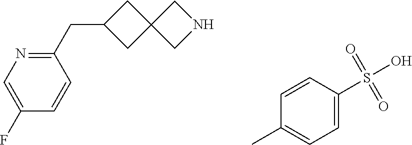 | 6-[(5-fluoro-2- pyridyl)methyl]-2- azaspiro[3.3]heptane; 4- methylbenzenesulfonic acid | 2-bromo- 5-fluoropyridine | 207.5 [M − TsOH + H]⁺ |
| B.23 | 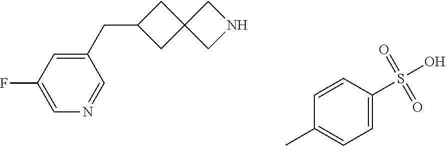 | 6-[(5-fluoro-3- pyridyl)methyl]-2- azaspiro[3.3]heptane; 4- methylbenzenesulfonic acid | 3-bromo- 5- fluoropyridine | 207.2 [M − TsOH + H]⁺ |
| B.24 | 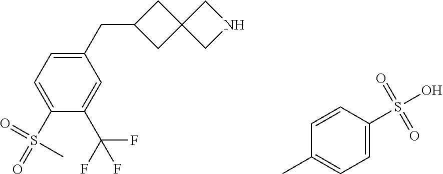 [SMILES: CS(=O)(=O)c1ccc(CC2CC3(CNC3)C2)cc1C(F)(F)F ; Cc1ccc(S(=O)(=O)O)cc1] | 6-[4-methylsulfonyl-3- (trifluoromethyl)phenyl] methyl]-2- azaspiro[3.3]heptane; 4- methylbenzenesulfonic acid | 4-bromo- 1- methylsulfonyl- 2- (trifluoromethyl) benzene (CAS: 300356- 32-5) | 334.2 [M − TsOH + H]⁺ |
| B.25 | 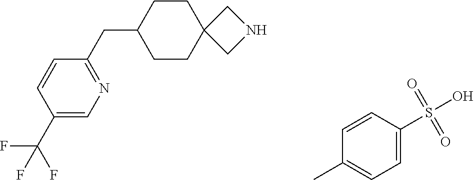 [SMILES: FC(F)(F)c1ccc(CC2CCC3(CC2)CNC3)nc1 ; Cc1ccc(S(=O)(=O)O)cc1] | 7-[[5-(trifluoromethyl)- 2-pyridyl]methyl]-2- azaspiro[3.5]nonane; 4- methylbenzenesulfonic acid | 2-bromo- 5- (trifluoromethyl) pyridine | 285.2 [M − TsOH + H]⁺ |
| B.26 | 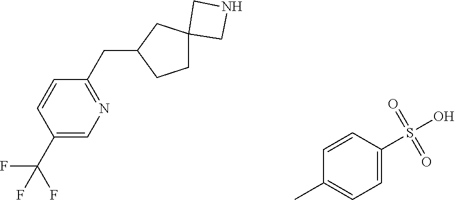 [SMILES: FC(F)(F)c1ccc(CC2CCC3(CNC3)C2)nc1 ; Cc1ccc(S(=O)(=O)O)cc1] | 6-[5-(trifluoromethyl)- 2-pyridyl]methyl]-2- azaspiro[3.4]octane; 4- methylbenzenesulfonic acid | 2-bromo- 5- (trifluoromethyl) pyridine | 271.2 [M − TsOH + H]⁺ |
| B.34 | 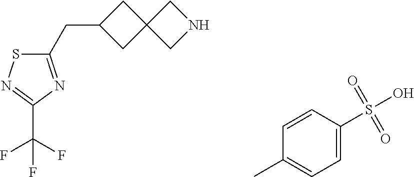 [SMILES: FC(F)(F)c1nsc(CC2CC3(CNC3)C2)n1 ; Cc1ccc(S(=O)(=O)O)cc1] | 5-(2- azaspiro[3.3]heptan-6- ylmethyl)-3- (trifluoromethyl)-1,2,4- thiadiazole; 4- methylbenzenesulfonic acid | 5-bromo-3- (trifluoro methyl)- 1,2,4- thiadiazole (CAS: 1781783- 65-0) | 264.0 [M − TsOH + H]⁺ |
| B.35 | 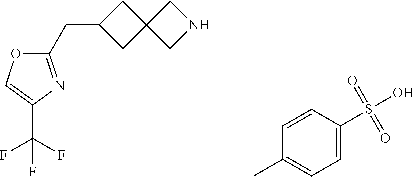 [SMILES: FC(F)(F)c1coc(CC2CC3(CNC3)C2)n1 ; Cc1ccc(S(=O)(=O)O)cc1] | 2-(2- azaspiro[3.3]heptan-6- ylmethyl)-4- (trifluoromethyl) oxazole; 4- methylbenzenesulfonic acid | 2-bromo-4- (trifluoromethyl) oxazole (CAS: 1060816- 14-9) | 247.2 [M − TsOH + H]⁺ |
| B.47 | 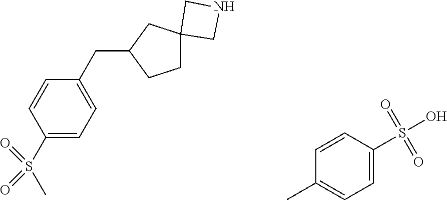 [SMILES: CS(=O)(=O)c1ccc(CC2CCC3(CNC3)C2)cc1 ; Cc1ccc(S(=O)(=O)O)cc1] | 6-[(4- methylsulfonylphenyl) methyl]-2- azaspiro[3.4]octane; 4- methylbenzenesulfonic acid | 4- bromophenyl- methylsulfone | 280.0 [M − TsOH + H]⁺ |
| B.69 | 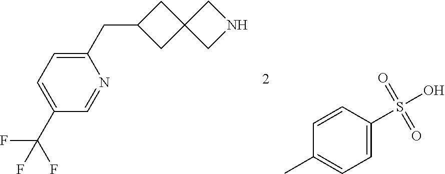 [SMILES: FC(F)(F)c1ccc(CC2CC3(CNC3)C2)nc1 ; Cc1ccc(S(=O)(=O)O)cc1] | 6-[[5-(trifluoromethyl)- 2-pyridyl]methyl]-2- azaspiro[3.3]heptane; di 4- methylbenzenesulfonic acid | 2-bromo-5- (trifluoromethyl) pyridine | 257.4 [M − TsOH + H]⁺ |
| B.70 | 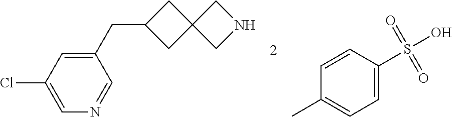 | 6-[(5-chloro-3- pyridyl)methyl]-2- azaspiro[3.3]heptane; di 4- methylbenzenesulfonic acid | 3-bromo- 5- chloropyridine | 223.1 [M − TsOH + H]⁺ |
| | *PtO₂ catalyst used in Step c) | | | |
| B.71 | 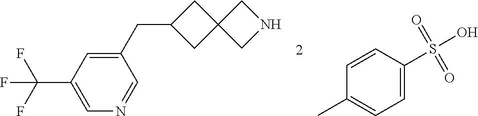 [SMILES: FC(F)(F)c1cncc(CC2CC3(CNC3)C2)c1 ; Cc1ccc(S(=O)(=O)O)cc1] | 6-[5-(trifluoromethyl)- 3-pyridyl]methyl]-2- azaspiro[3.3]heptane; di 4- methylbenzenesulfonic acid | 3-bromo-5- (trifluoro methyl) pyridine | 257.4 [M − TsOH + H]⁺ |
| B.72 | 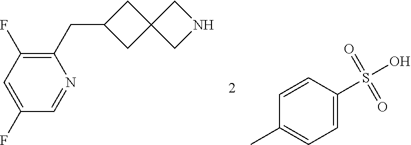 [SMILES: Fc1cnc(CC2CC3(CNC3)C2)c(F)c1 ; Cc1ccc(S(=O)(=O)O)cc1] | 6-[(3,5-difluoro-2- pyridyl)methyl]-2- azaspiro[3.3]heptane; di 4- methylbenzenesulfonic acid | 2-bromo-3,5- difluoropyridine | 225.0 [M − TsOH + H]⁺ |
| B.73 | 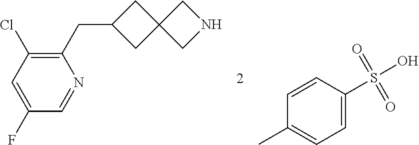 [SMILES: Fc1cnc(CC2CC3(CNC3)C2)c(Cl)c1 ; Cc1ccc(S(=O)(=O)O)cc1] | 6-[(3-chloro-5-fluoro-2- pyridyl)methyl]-2- azaspiro[3.3]heptane;di 4- methylbenzenesulfonic acid | 2-bromo- 3-chloro- 5-fluoro- pyridine | 241.1 [M − TsOH + H]⁺ |
| B.74 | 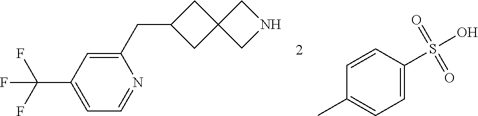 [SMILES: FC(F)(F)c1ccnc(CC2CC3(CNC3)C2)c1 ; Cc1ccc(S(=O)(=O)O)cc1] | 6-[[4-(trifluoromethyl)- 2-pyridyl]methyl]-2- azaspiro[3.3]heptane; di 4- methylbenzenesulfonic acid; | 2-bromo-4- (trifluoromethyl) pyridine | 257.5 [M − TsOH + H]⁺ |
| B.75 | 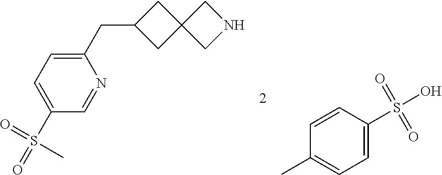 [SMILES: CS(=O)(=O)c1ccc(CC2CC3(CNC3)C2)nc1 ; Cc1ccc(S(=O)(=O)O)cc1] | 6-[(5-methylsulfonyl-2- pyridyl)methyl]-2- azaspiro[3.3]heptane; di 4- methylbenzenesulfonic acid | 2-bromo- 5- (methylsulfonyl) pyridine (CAS: 343262- 51-1) | 267.2 [M − TsOH + H]⁺ |
| B.76 | 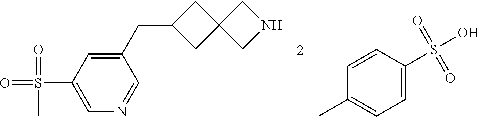 [SMILES: CS(=O)(=O)c1cncc(CC2CC3(CNC3)C2)c1 ; Cc1ccc(S(=O)(=O)O)cc1] | 6-[(5-methylsulfonyl-3- pyridyl)methyl]-2- azaspiro[3.3]heptane; 4- methylbenzenesulfonic acid | 3-bromo-5- (methylsulfonyl) pyridine (CAS: 445491- 71-4) | 267.2 [M − 2TsOH + H]⁺ |
| B.77 | 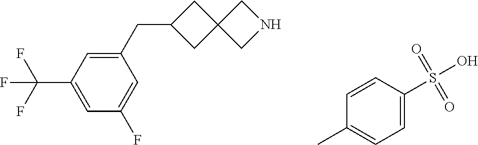 [SMILES: Fc1cc(CC2CC3(CNC3)C2)cc(C(F)(F)F)c1 ; Cc1ccc(S(=O)(=O)O)cc1] | 6-[[3-fluoro-5- (trifluoromethyl)phenyl] methyl]-2- azaspiro[3.3]heptane; 4- methylbenzenesulfonic acid | 3-bromo- 5- fluoro- benzotrifluoride | 274.0 [M − TsOH + H]⁺ |
| B.78 | 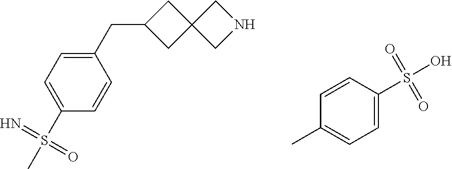 [SMILES: CS(=N)(=O)c1ccc(CC2CC3(CNC3)C2)cc1 ; Cc1ccc(S(=O)(=O)O)cc1] | [4-(2- azaspiro[3.3]heptan-6- ylmethyl)phenyl]- imino-methyl-oxo-A6- sulfane; 4- methylbenzenesulfonic acid | 1-bromo-4-(S- methyl- sulfonimidoyl) benzene (CAS: 145026- 07-9) | 265.2 [M − TsOH + H]⁺ |
| B.79 | 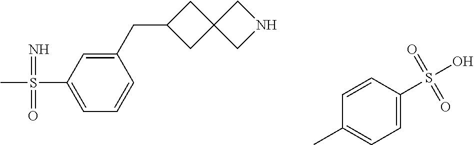 [SMILES: CS(=N)(=O)c1cccc(CC2CC3(CNC3)C2)c1 ; Cc1ccc(S(=O)(=O)O)cc1] | [3-(2- azaspiro[3.3]heptan-6- ylmethyl)phenyl]- imino-methyl-oxo-26- sulfane; 4- methylbenzenesulfonic acid | (3- bromophenyl) (imino) (methyl)-16- sulfanone (CAS: 1789700- 09-9) | 265.2 [M − TsOH + H]⁺ |
| B.80 | 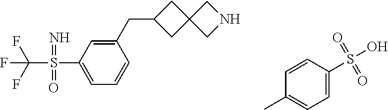 [SMILES: N=S(=O)(c1cccc(CC2CC3(CNC3)C2)c1)C(F)(F)F ; Cc1ccc(S(=O)(=O)O)cc1] | [3-(2- azaspiro[3.3]heptan-6- ylmethyl)phenyl]- imino-oxo- (trifluoromethyl)-λ⁶- sulfane; 4- methylbenzenesulfonic acid | (3- bromophenyl) (imino) (trifluoromethyl)- λ⁶-sulfanone (CAS: 2411289-54-6) | 319.0 [M − TsOH + H]⁺ |
| B.81 | 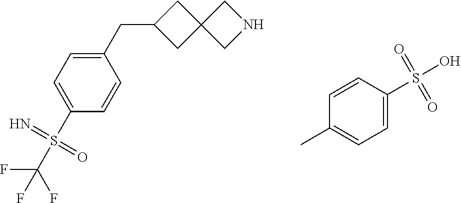 [SMILES: N=S(=O)(c1ccc(CC2CC3(CNC3)C2)cc1)C(F)(F)F ; Cc1ccc(S(=O)(=O)O)cc1] | [4-(2- azaspiro[3.3]heptan-6- ylmethyl)phenyl]- imino-oxo- (trifluoromethyl)-λ⁶- sulfane; 4- methylbenzenesulfonic acid | (4- bromophenyl) (imino) (trifluoromethyl)- λ6- sulfanone (CAS: 1421133- 03-0) | 319.2 [M − TsOH + H]⁺ |
| B.82 | 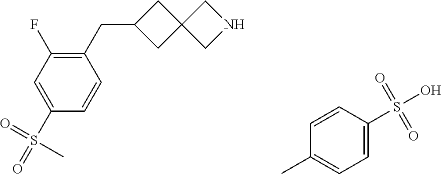 [SMILES: CS(=O)(=O)c1ccc(CC2CC3(CNC3)C2)c(F)c1 ; Cc1ccc(S(=O)(=O)O)cc1] | 6-[(2-fluoro-4- methylsulfonyl- phenyl)methyl]-2- azaspiro[3.3]heptane; 4- methylbenzenesulfonic acid | 1-bromo- 2-fluoro-4- methyl- sulfonylbenzene (CAS: 1032825- 02-7) | 284.1 [M − TsOH + H]⁺ |
| B.86 | 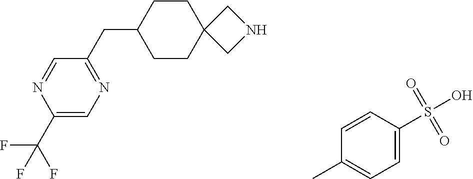 [SMILES: FC(F)(F)c1cnc(CC2CCC3(CC2)CNC3)cn1 ; Cc1ccc(S(=O)(=O)O)cc1] | 7-[[5- (trifluoromethyl) pyrazin-2-yl] methyl]-2- azaspiro[3.5] nonane; 4- methylbenzenesulfonic acid | 2-bromo-5- (trifluoromethyl) pyrazine | 285.0 [M − TsOH + H]⁺ |
| B.87 | 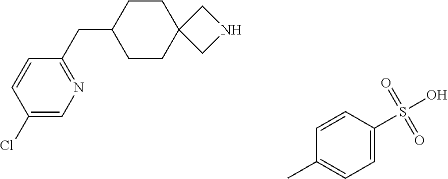 [SMILES: Clc1ccc(CC2CCC3(CC2)CNC3)nc1 ; Cc1ccc(S(=O)(=O)O)cc1] | 7-[(5-chloro-2- pyridyl)methyl]-2- azaspiro[3.5]nonane; 4- methylbenzenesulfonic acid | 2-bromo- 5- chloropyridine | 251.2 [M − TsOH + H]⁺ |
| | *Rh(10% on C) catalyst used in | | | |
| | Step c) | | | |
| B.88 | 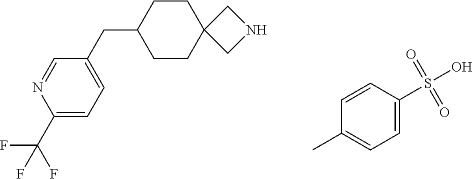 [SMILES: FC(F)(F)c1ccc(CC2CCC3(CC2)CNC3)cn1 ; Cc1ccc(S(=O)(=O)O)cc1] | 7-[[6-(trifluoromethyl)- 3-pyridyl]methyl]-2- azaspiro[3.5]nonane; 4- methylbenzenesulfonic acid | 5-bromo-2- (trifluoromethyl) pyridine | 285.2 [M − TsOH + H]⁺ |
| B.89 | 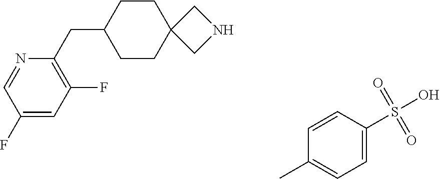 [SMILES: Fc1cnc(CC2CCC3(CC2)CNC3)c(F)c1 ; Cc1ccc(S(=O)(=O)O)cc1] | 7-[(3,5-difluoro-2- pyridyl)methyl]-2- azaspiro[3.5]nonane; 4- methylbenzenesulfonic acid | 2-bromo- 3,5- difluoropyridine | 253.2 [M − TsOH + H]⁺ |
| B.90 | 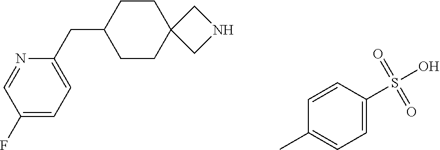 [SMILES: Fc1ccc(CC2CCC3(CC2)CNC3)nc1 ; Cc1ccc(S(=O)(=O)O)cc1] | 7-[(5-fluoro-2- pyridyl)methyl]-2- azaspiro[3.5]nonane; 4- methylbenzenesulfonic acid | 2-bromo- 5- fluoropyridine | 235.4 [M − TsOH + H]⁺ |
| B.91 | 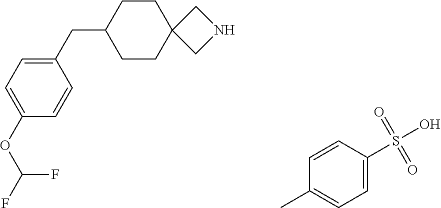 [SMILES: FC(F)Oc1ccc(CC2CCC3(CC2)CNC3)cc1 ; Cc1ccc(S(=O)(=O)O)cc1] | 6-[[4- (difluoromethoxy) phenyl]methyl]-2- azaspiro[3.3]heptane; 4- methylbenzenesulfonic acid | 5-bromo- 2- (difluoromethoxy) pyridine (CAS: 899452- 26-7) | 283.2 [M − TsOH + H]⁺ |
| B.92 | 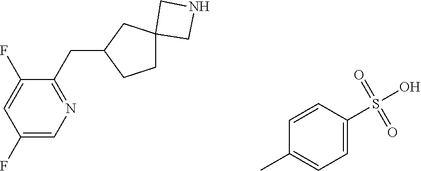 [SMILES: Fc1cnc(CC2CCC3(CNC3)C2)c(F)c1 ; Cc1ccc(S(=O)(=O)O)cc1] | 6-[(3,5-difluoro-2- pyridyl)methyl]-2- azaspiro[3.4]octane; 4- methylbenzenesulfonic acid | 2-bromo- 3,5- difluoropyridine | 239.2 [M − TsOH + H]⁺ |
| B.93 | 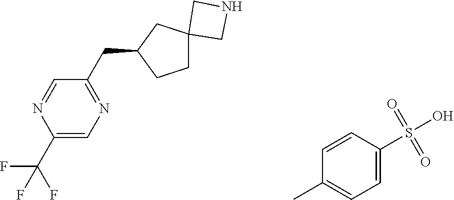 [SMILES: FC(F)(F)c1cnc(C[C@@H]2CCC3(CNC3)C2)cn1 ; Cc1ccc(S(=O)(=O)O)cc1] | (6R)-6-[[5- (trifluoromethyl) pyrazin-2-yl]methyl]-2- azaspiro[3.4]octane; 4- methylbenzenesulfonic acid | 2-bromo- 5- (trifluoromethyl) pyrazine | 272.2 [M − TsOH + H]⁺ |
| | (arbitrary assignment of | | | |
| | stereochemistry, separated by | | | |
| | chiral SFC after Step c ) | | | |
| B.94 | 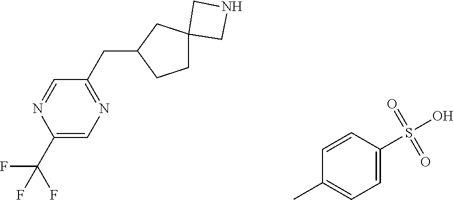 [SMILES: FC(F)(F)c1cnc(CC2CCC3(CNC3)C2)cn1 ; Cc1ccc(S(=O)(=O)O)cc1] | 6-[[5- (trifluoromethyl) pyrazin-2-yl]methyl]-2- azaspiro[3.4]octane; 4- methylbenzenesulfonic acid | 2-bromo-5- (trifluoromethyl) pyrazine | 272.2 [M − TsOH + H]⁺ |
| | (arbitrary assignment of | | | |
| | stereochemistry, separated by | | | |
| | chiral SFC after Step c ) | | | |
| B.95 | 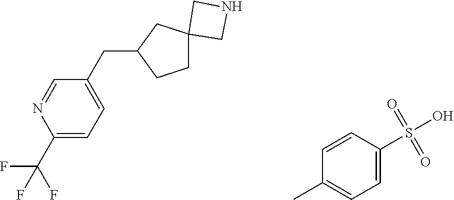 [SMILES: FC(F)(F)c1ccc(CC2CCC3(CNC3)C2)cn1 ; Cc1ccc(S(=O)(=O)O)cc1] | 6-[[6-(trifluoromethyl)- 3-pyridyl]methyl]-2- azaspiro[3.4]octane; 4- methylbenzenesulfonic acid | 5-bromo- 2- (trifluoromethyl) pyridine | 271.1 [M − TsOH + H]⁺ |
| B.96 | 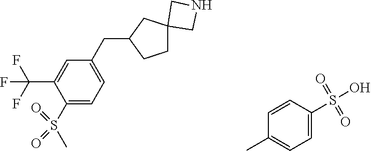 [SMILES: CS(=O)(=O)c1ccc(CC2CCC3(CNC3)C2)cc1C(F)(F)F ; Cc1ccc(S(=O)(=O)O)cc1] | 6-[[4-methylsulfonyl-3- (trifluoromethyl)phenyl] methyl]-2- azaspiro[3.4]octane; 4- methylbenzenesulfonic acid | 4-bromo- 1-mesyl- 2- (trifluoromethyl) benzene (CAS: 300356- 32-5) | 348.3 [M − TsOH + H]⁺ |
| B.302 | 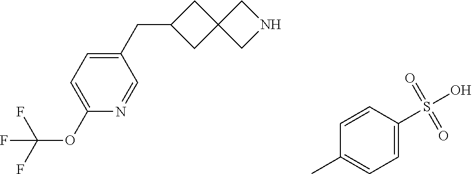 [SMILES: FC(F)(F)Oc1ccc(CC2CC3(CNC3)C2)cn1 ; Cc1ccc(S(=O)(=O)O)cc1] | 6-[6- (trifluoromethoxy)-3- pyridyl]methyl]-2- azaspiro[3.3]heptane; 4- methylbenzenesulfonic acid | 5-bromo-2- (trifluoro methoxy) pyridine (CAS: 886371- 77-3) | 273.3 [M − TsOH + H]⁺ |
| B.305 | 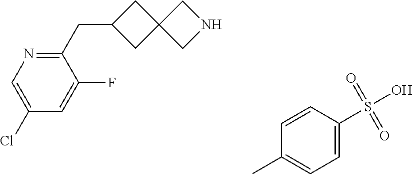 [SMILES: Fc1cc(Cl)cnc1CC1CC2(CNC2)C1 ; Cc1ccc(S(=O)(=O)O)cc1] | 6-[(5-chloro-3-fluoro-2- pyridyl)methyl]-2- azaspiro[3.3]heptane; di 4- methylbenzenesulfonic acid | 2-bromo- 5-chloro- 3-fluoro- pyridine (CAS: 514797- 97-8) | 241.1 [M − 2TsOH + H]⁺ |
| B.306 | 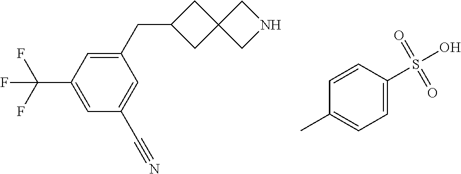 [SMILES: N#Cc1cc(CC2CC3(CNC3)C2)cc(C(F)(F)F)c1 ; Cc1ccc(S(=O)(=O)O)cc1] | 3-(2- azaspiro[3.3]heptan-6- ylmethyl)-5- (trifluoromethyl) benzonitrile; 4- methylbenzenesulfonic acid | 3-bromo- 5- (trifluoromethyl) benzonitrile (CAS: 691877- 03-9) | 281.2 [M − TsOH + H]⁺ |
| B.307 | 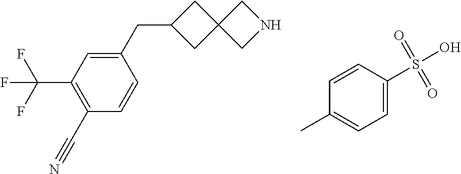 [SMILES: N#Cc1ccc(CC2CC3(CNC3)C2)cc1C(F)(F)F ; Cc1ccc(S(=O)(=O)O)cc1] | 4-(2- azaspiro[3.3]heptan-6- ylmethyl)-2- (trifluoromethyl) benzonitrile; di 4- methylbenzenesulfonic acid | 4-bromo- 2- (trifluoromethyl) benzonitrile (CAS: 191165- 13-6) | 281.1 [M − 2TsOH + H]⁺ |
| B.308 | 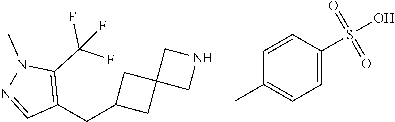 [SMILES: Cn1ncc(CC2CC3(CNC3)C2)c1C(F)(F)F ; Cc1ccc(S(=O)(=O)O)cc1] | 6-[[1-methyl-5- (trifluoromethyl) pyrazol- 4-yl]methyl]-2- azaspiro[3.3]heptane; 4- methylbenzenesulfonic acid | 4-bromo-1- methyl- 5- (trifluoromethyl) pyrazole | 260.2 [M − TsOH + H]⁺ |
| B.309 | 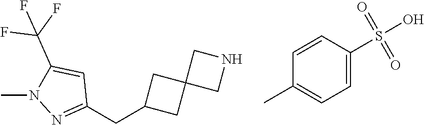 [SMILES: Cn1nc(CC2CC3(CNC3)C2)cc1C(F)(F)F ; Cc1ccc(S(=O)(=O)O)cc1] | 6-[1-methyl-5- (trifluoromethyl) pyrazol- 3-yl]methyl]-2- azaspiro[3.3]heptane; 4- methylbenzenesulfonic acid | 3-bromo-1- methyl- 5- (trifluoromethyl) pyrazole | 260.0 [M − TsOH + H]⁺ |
| B.310 | 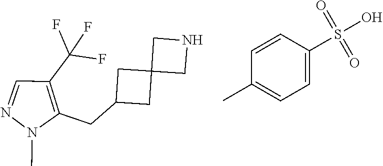 [SMILES: Cn1ncc(C(F)(F)F)c1CC1CC2(CNC2)C1 ; Cc1ccc(S(=O)(=O)O)cc1] | 6-[2-methyl-4- (trifluoromethyl)pyrazol- 3-yl]methyl]-2- azaspiro[3.3]heptane; 4- methylbenzenesulfonic acid; | 5-bromo-1- methyl-4- (trifluoromethyl) pyrazole (CAS: 1783749- 68-7) | 260.0 [M − TsOH + H]⁺ |
| B.312 | 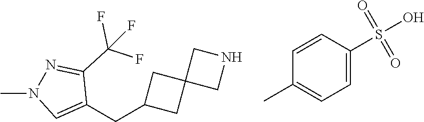 [SMILES: Cn1cc(CC2CC3(CNC3)C2)c(C(F)(F)F)n1 ; Cc1ccc(S(=O)(=O)O)cc1] | 6-[1-methyl-3- (trifluoromethyl) pyrazo 1-4-yl]methyl]-2- azaspiro[3.3]heptane; 4- methylbenzenesulfonic acid | 4-bromo-1- methyl-3- (trifluoro methyl)- 1H-pyrazole (CAS: 497832-99-2) | 260.2 [M − TsOH + H]⁺ |
| B.313 | 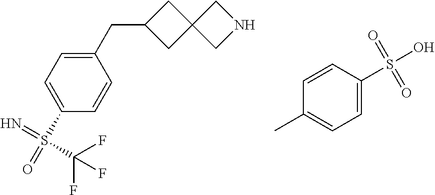 | [4-(2- azaspiro[3.3]heptan-6- ylmethyl)phenyl]- imino-oxo- (trifluoromethyl)-λ⁶- sulfane; 4- methylbenzenesulfonic acid | (4- bromophenyl) (imino) (trifluoromethyl)- λ6- sulfanone (CAS: 1421133-03-0) | 319.1 [M − TsOH + H]⁺ |
| | (arbitrary assignment of | | | |
| | stereochemistry, separated by | | | |
| | chiral SFC after Step c) | | | |
| B.314 | 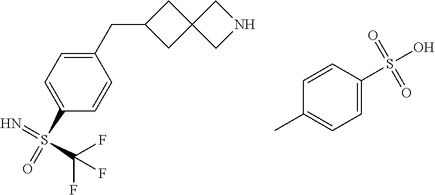 [SMILES: N=[S@](=O)(c1ccc(CC2CC3(CNC3)C2)cc1)C(F)(F)F ; Cc1ccc(S(=O)(=O)O)cc1] | [4-(2- azaspiro[3.3.]heptan-6- ylmethyl)phenyl]- imino-oxo- (trifluoromethyl)-λ⁶- sulfane; 4- methylbenzenesulfonic acid | (4- bromophenyl) (imino) (trifluoromethyl)- λ6- sulfanone (CAS: 1421133-03-0) | 319.3 [M − TsOH + H]⁺ |
| | (arbitrary assignment of | | | |
| | stereochemistry, separated by | | | |
| | chiral SFC after Step c) | | | |
| P.77 | 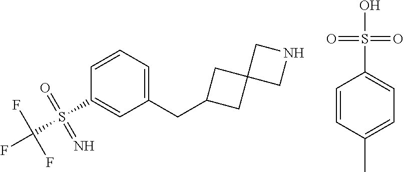 | [3-(2- azaspiro[3.3]heptan-6- ylmethyl)phenyl]- imino-oxo- (trifluoromethyl)-λ⁶- sulfane; 4- methylbenzenesulfonic acid | (3- bromophenyl)- imino- oxo- (trifluoro methyl)- λ 6- sulfane | 319.1 [M + H]⁺ |
| | (arbitrary assignment of | | | |
| | stereochemistry, separated by | | | |
| | chiral SFC after Step c ) | | | |
| P.78 | 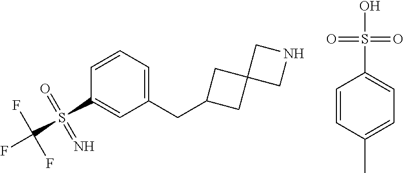 | [3-(2- azaspiro[3.3]heptan-6- ylmethyl)phenyl]- imino-oxo- (trifluoromethyl)-λ⁶- sulfane; 4- methylbenzenesulfonic acid | (3- bromophenyl)- imino- oxo- (trifluoro methyl)- λ 6- sulfane | 319.1 [M + H]⁺ |
| | (arbitrary assignment of | | | |
| | stereochemistry, separated by | | | |
| | chiral SFC after Step c ) | | | |
| P.41 | 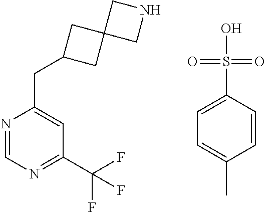 [SMILES: FC(F)(F)c1cc(CC2CC3(CNC3)C2)ncn1 ; Cc1ccc(S(=O)(=O)O)cc1] | 4- methylbenzenesulfonic acid;6-[[6- (trifluoromethyl) pyrimidin- 4-yl]methyl]-2- azaspiro[3.3]heptane | 4-chloro-6- (trifluoromethyl) pyrimidine | 258.0 [M + H]⁺ |
| P.42 | 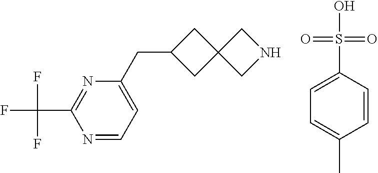 | 4- methylbenzenesulfonic acid;6-[[2- (trifluoromethyl) pyrimidin- 4-yl]methyl]-2- azaspiro[3.3]heptane | 4-chloro- 2- (trifluoromethyl) pyrimidine | 258.1 [M + H]⁺ |
| P.7 | 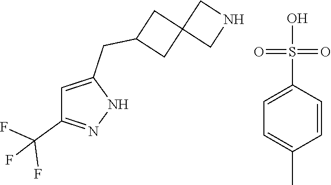 | 6-[3-(trifluoromethyl)- 1H-pyrazol-5- yl]methyl]-2- azaspiro[3.3]heptane; 4- methylbenzenesulfonic acid | 5-bromo-3- (trifluoromethyl)- 1H- pyrazole | 246.0 [M + H]⁺ |
| P.10 | 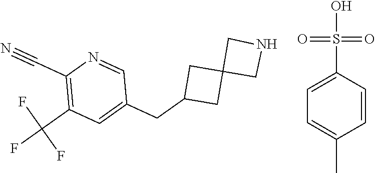 | 5-(2- azaspiro[3.3]heptan-6- ylmethyl)-3- (trifluoromethyl) picolinonitrile; 4- methylbenzenesulfonic acid | 5-bromo-3- (trifluoromethyl) picolinonitrile (CAS 1214377- 57-7) | 282.2 [M + H]⁺ |
| P.11 | 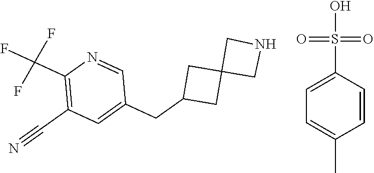 | 5-(2- azaspiro[3.3]heptan-6- ylmethyl)-2- (trifluoromethyl)nicotin onitrile; 4- methylbenzenesulfonic acid | 5-bromo- 2- (trifluoromethyl) nicotinonitrile (CAS 1245914- 98-0) | 282.2 [M + H]⁺ |
| P.12 | 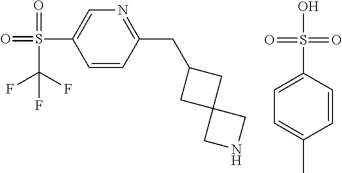 | 6-[[5- (trifluoromethyl- sulfonyl)- 2-pyridyl]methyl]-2- azaspiro[3.3]heptane; 4- methylbenzenesulfonic acid | 5-chloro- 2- (trifluoro- methylsulfonyl) pyridine (CAS: 2091447- 89-9) | 321.1 [M + H]⁺ |
| P.13 | 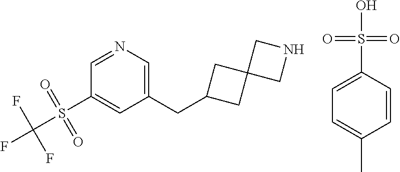 | 4- methylbenzenesulfonic acid;6-[[5- (trifluoromethyl- sulfonyl)- 3-pyridyl]methyl]-2- azaspiro[3.3]heptane | 3-bromo- 5- (trifluoro methylsulfonyl) pyridine (CAS: 1936542- 46-9) | 321.1 [M + H]⁺ |
| P.31 | 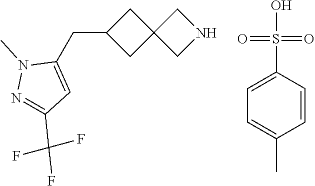 | 6-[[2-methyl-5- (trifluoromethyl)pyrazol- 3-yl]methyl]-2- azaspiro[3.3]heptane; 4- methylbenzenesulfonic acid | 5-bromo- 1- methyl- 3- (trifluoromethyl) pyrazole | 260.2 [M + H]⁺ |
| P.34 | 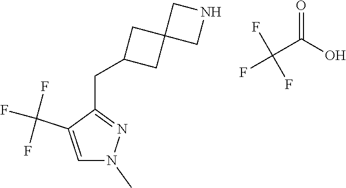 [SMILES: Cn1cc(C(F)(F)F)c(CC2CC3(CNC3)C2)n1 ; O=C(O)C(F)(F)F] | 6-[1-methyl-4- (trifluoromethyl)pyrazo 1-3-yl]methyl]-2- azaspiro[3.3]heptane; 2,2,2-trifluoroacetic acid | 3-bromo- 1- methyl- 4- (trifluoromethyl) pyrazole | 260.1 [M + H]⁺ |
| P.37 | 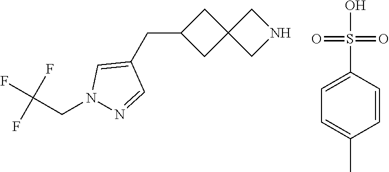 | 6-[1-(2,2,2- trifluoroethyl)pyrazol- 4-yl]methyl]-2- azaspiro[3.3]heptane; 4- methylbenzenesulfonic acid | 4-bromo- 1-(2,2,2- trifluoroethyl) pyrazole | 246.0 [M + H]⁺ |
| P.66 | 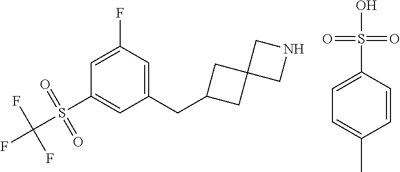 | 6-[[3-fluoro-5- (trifluoromethylsulfonyl) phenyl]methyl]-2- azaspiro[3.3]heptane;4- methylbenzenesulfonic acid | 1-bromo- 3-fluoro- 5- (trifluoromethyl- sulfonyl) benzene | 338.0 [M + H]⁺ |
| P.74 | 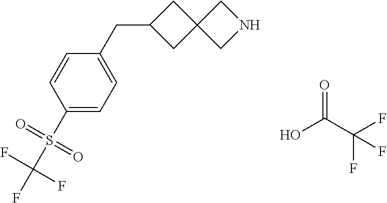 [SMILES: O=S(=O)(c1ccc(CC2CC3(CNC3)C2)cc1)C(F)(F)F ; O=C(O)C(F)(F)F] | 6-[[4- (trifluoromethyl- sulfonyl) phenyl]methyl]-2- azaspiro[3.3]heptane; 2,2,2-trifluoroacetic acid | 1-bromo-4- (trifluoro- methylsulfonyl) benzene | 320.1 [M + H]⁺ |
| P.90 | 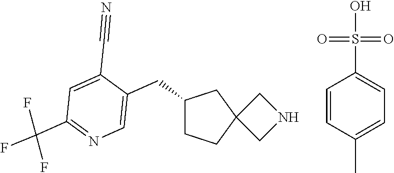 | 5-[[rac-(6S)-2- azaspiro[3.4]octan-6- yl]methyl]-2- (trifluoromethyl) pyridine- 4-carbonitrile; 4- methylbenzenesulfonic acid | 5-bromo-2- (trifluoromethyl) pyridine- 4- carbonitrile (CAS: 1070892- 04-4) | 296.2 [M + H]⁺ |
| | (arbitrary assignment of | | | |
| | stereochemistry, separated by | | | |
| | chiral SFC after Step c ) | | | |
| P.91 | 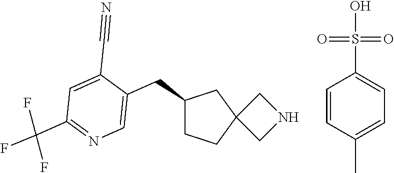 | 5-[[rac-(6R)-2- azaspiro[3.4]octan-6- yl]methyl]-2- (trifluoromethyl) pyridine-4- carbonitrile; 4- methylbenzenesulfonic acid | 5-bromo-2- (trifluoromethyl) pyridine- 4- carbonitrile (CAS: 1070892- 04-4) | 296.2 [M + H]⁺ |
| | (arbitrary assignment of | | | |
| | stereochemistry, separated by | | | |
| | chiral SFC after Step c ) | | | |
| P.95 | 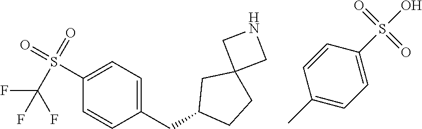 | (6R)-6-[[4- (trifluoro- methylsulfonyl) phenyl]methyl]-2- azaspiro[3.4]octane; 4- methylbenzenesulfonic acid | 1-bromo-4- (trifluoro- methylsulfonyl) benzene | 334.2 [M + H]⁺ |
| | (arbitrary assignment of | | | |
| | stereochemistry, separated by | | | |
| | chiral HPLC after Step c) | | | |
| P.96 | 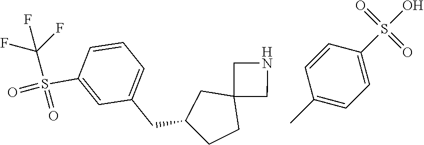 | (6R)-6-[[3- (trifluoromethylsulfonyl) phenyl]methyl]-2- azaspiro[3.4]octane; 4- methylbenzenesulfonic acid; | 1-bromo-3- (trifluoro- methylsulfonyl) benzene | 334.1 [M + H]⁺ |
| | (arbitrary assignment of | | | |
| | stereochemistry, separated by | | | |
| | chiral HPLC after Step c) | | | |
| B.315 | 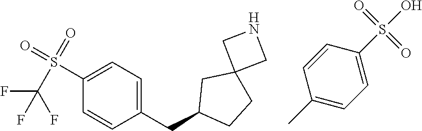 | (6S)-6-[[4- (trifluoro- methylsulfonyl) phenyl]methyl]-2- azaspiro[3.4]octane; 4- methylbenzenesulfonic acid | 1-bromo- 4- (trifluoro- methylsulfonyl) benzene | 334.2 [M + H]⁺ |
| | (arbitrary assignment of | | | |
| | stereochemistry, separated by | | | |
| | chiral HPLC after Step c) | | | |
| B.316 | 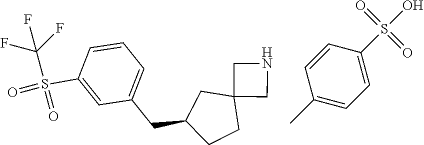 | (6S)-6-[[3- (trifluoromethylsulfonyl) phenyl methyl]-2- azaspiro[3.4]octane; 4- methylbenzenesulfonic acid; | 1-bromo-3- (trifluoro- methylsulfonyl) benzene | 334.1 [M + H]⁺ |
| | (arbitrary assignment of | | | |
| | stereochemistry, separated by | | | |
| | chiral HPLC after Step c) | | | |
| P.71 | 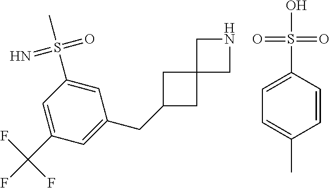 | [3-(2- azaspiro[3.3]heptan-6- ylmethyl)-5- (trifluoromethyl) phenyl]- imino-methyl-oxo-λ⁶- sulfane; 4- methyl- benzenesulfonic acid | C.6 [3- bromo-5- (trifluoromethyl) phenyl]- imino- methyl- oxo-λ6- sulfane | 333.2 [M + H]⁺ |
| B.318 | 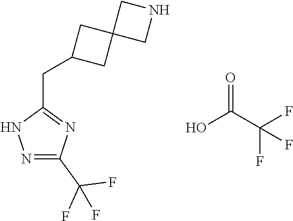 [SMILES: FC(F)(F)c1n[nH]c(CC2CC3(CNC3)C2)n1 ; O=C(O)C(F)(F)F] | 6-[[3-(trifluoromethyl)- 1H-1,2,4-triazol-5- yl]methyl]-2- azaspiro[3.3]heptane; 2,2,2-trifluoroacetic acid | 3-Bromo-5- (trifluoro methyl)- IH- 1,2,4- triazole (CAS: 1185320-36-8) | 247.5 [M − TFA + H]⁺ |
| B.321 | 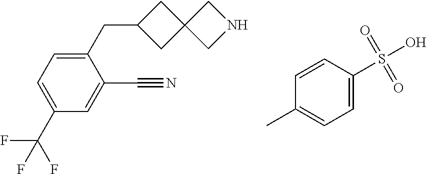 [SMILES: N#Cc1cc(C(F)(F)F)ccc1CC1CC2(CNC2)C1 ; Cc1ccc(S(=O)(=O)O)cc1] | 2-(2-azaspiro [3.3]heptan-6- ylmethyl)-5- (trifluoromethyl) benzonitrile; 4- methyl- benzenesulfonic acid | 2-bromo-5- (trifluoromethyl) benzonitrile (CAS: 1483-55-2) | 281.1 [M − TsOH + H]⁺ |
| B.322 | 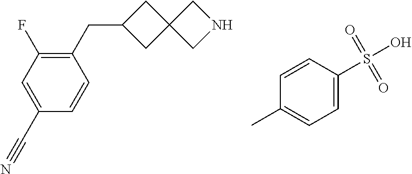 [SMILES: N#Cc1ccc(CC2CC3(CNC3)C2)c(F)c1 ; Cc1ccc(S(=O)(=O)O)cc1] | 4-(2- azaspiro[3.3]heptan-6- ylmethyl)-3-fluoro- benzonitrile; 4- methylbenzenesulfonic acid | 4-bromo- 3- fluorobenzonitrile | 231.2 [M − TsOH + H]⁺ |
| B.330 | 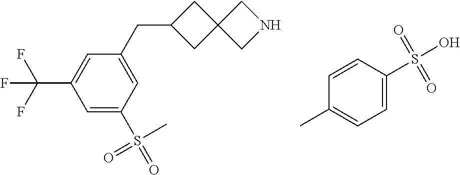 [SMILES: CS(=O)(=O)c1cc(CC2CC3(CNC3)C2)cc(C(F)(F)F)c1 ; Cc1ccc(S(=O)(=O)O)cc1] | 6-[[3-methylsulfonyl-5- (trifluoromethyl)phenyl] methyl]-2- azaspiro[3.3]heptane; 4- methylbenzenesulfonic acid | 1-bromo-3- methylsulfonyl- 5- (trifluoromethyl) benzene (CAS: 1215205- 96-1) | 334.1 [M − TsOH + H]⁺ |
| B.331 | 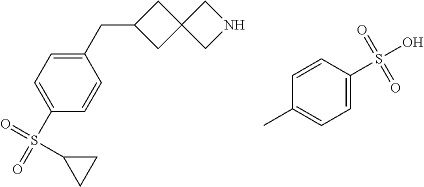 [SMILES: O=S(=O)(c1ccc(CC2CC3(CNC3)C2)cc1)C1CC1 ; Cc1ccc(S(=O)(=O)O)cc1] | 6-[(4- cyclopropyl- sulfonylphenyl) methyl]-2- azaspiro[3.3]heptane; 4- methylbenzenesulfonic acid | 1-bromo-4- cyclopropyl- sulfonyl- benzene (CAS: 648906- 28-9) | 292.2 [M − TsOH + H]⁺ |
| B.335 | 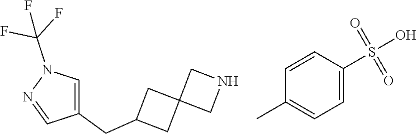 [SMILES: FC(F)(F)n1cc(CC2CC3(CNC3)C2)cn1 ; Cc1ccc(S(=O)(=O)O)cc1] | 6-[[1- (trifluoromethyl) pyrazo 1-4-yl]methyl]-2- azaspiro[3.3]heptane; 4- methylbenzenesulfonic acid | 4-bromo- 1- (trifluoromethyl) pyrazole (CAS: 1046831- 97-3) | 246.1 [M − TsOH + H]⁺ |
| B.345 | 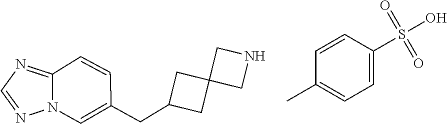 [SMILES: c1nc2ccc(CC3CC4(CNC4)C3)cn2n1 ; Cc1ccc(S(=O)(=O)O)cc1] | 6-(2- azaspiro[3.3]heptan-6- ylmethyl)- [1,2,4]triazolo[1,5- a]pyridine; di 4- methylbenzenesulfonic acid | 6-bromo- [1,2,4] triazolo[1,5- a]pyridine (CAS: 356560- 80-0) | 229.1 [M − TsOH + H]⁺ |
| B.346 | 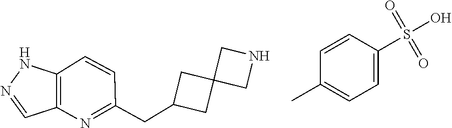 [SMILES: c1cc2[nH]ncc2nc1CC1CC2(CNC2)C1 ; Cc1ccc(S(=O)(=O)O)cc1] | 5-(2- azaspiro[3.3]heptan-6- ylmethyl)-1H- pyrazolo[4,3- b]pyridine;4- methylbenzenesulfonic acid | 5-bromo-1H- pyrazolo[4,3- b]pyridine (CAS: 1227628-78-5) | 229.1 [M − 2TsOH + H]⁺ |
| B.353 | 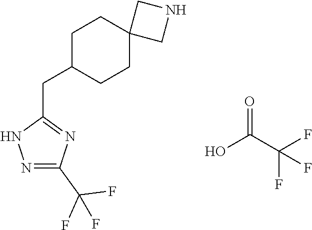 [SMILES: FC(F)(F)c1n[nH]c(CC2CCC3(CC2)CNC3)n1 ; O=C(O)C(F)(F)F] | 7-[[3-(trifluoromethyl)- 1H-1,2,4-triazol-5- yl]methyl]-2- azaspiro[3.5]nonane; 2,2,2-trifluoroacetic acid | 3-Bromo- 5- (trifluoro methyl)- 1H- 1,2,4- triazole (CAS: 1185320- 36-8) | 275.2 [M − TsOH + H]⁺ |
| B.355 | 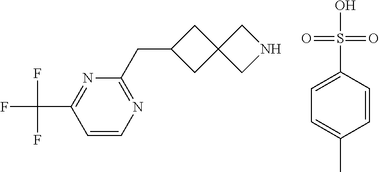 | 6-[[4- (trifluoromethyl) pyrimidin- 2-yl]methyl]-2- azaspiro[3.3]heptane; 4- methylbenzenesulfonic acid | 2-chloro-4- (trifluoromethyl) pyrimidine | 258.2 [M − TsOH + H]⁺ |
| B.356 | 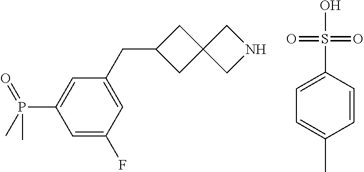 | 6-[(3- dimethylphosphoryl-5- fluoro-phenyl)methyl]- 2-azaspiro[3.3]heptane; 4- methylbenzenesulfonic acid | 1-bromo- 3- dimethyl- phosphoryl-5- fluoro- benzene (C.7) | 282.0 [M − TsOH + H]⁺ |
| B.357 | 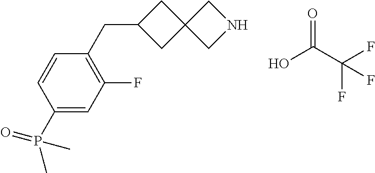 [SMILES: CP(C)(=O)c1ccc(CC2CC3(CNC3)C2)c(F)c1 ; O=C(O)C(F)(F)F] | 6-[(4- dimethylphosphoryl-2- fluoro-phenyl)methyl]- 2-azaspiro[3.3]heptane; 4- methylbenzenesulfonic acid | 1-chloro-4- dimethyl- phosphoryl- 2- fluoro- benzene (C.8) | 282.1 [M − TFA + H]⁺ |
| B.374 | 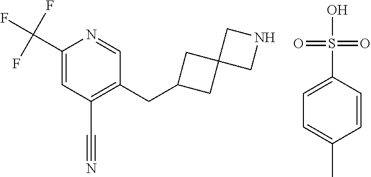 | 5-(2- azaspiro[3.3]heptan-6- ylmethyl)-2- (trifluoromethyl) pyridine-4- carbonitrile; 4- methylbenzenesulfonic acid | 5-Bromo-2- (trifluoromethyl) isonicotinonitrile (CAS: 1070892- 04-4) | 282.1 [M − TsOH + H]⁺ |
| B.398 | 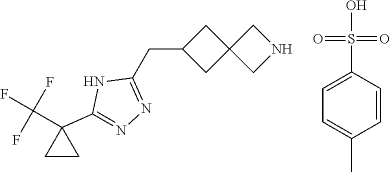 | 6-[[5-[1- (trifluoromethyl) cyclopropyl]-4H- 1,2,4-triazol- 3-yl]methyl]-2- azaspiro[3.3]heptane; 4- methylbenzenesulfonic acid | 3-bromo-5-[1- (trifluoromethyl) cyclopropyl]-IH- 1,2,4- triazole (C.10) | 287.1 [M − TsOH + H]⁺ |
| B.401 | 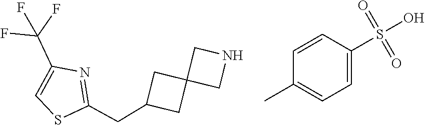 [SMILES: FC(F)(F)c1csc(CC2CC3(CNC3)C2)n1 ; Cc1ccc(S(=O)(=O)O)cc1] | 2-(2- azaspiro[3.3]heptan-6- ylmethyl)-4- (trifluoromethyl) thiazole; 4- methylbenzenesulfonic acid | 2-bromo- 4- (trifluoromethyl) thiazole (CAS: 41731- 39-9) | 263.0 [M − TsOH + H]⁺ |
| B.407 | 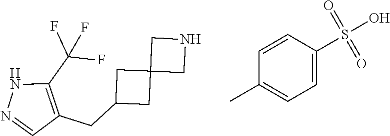 [SMILES: FC(F)(F)c1[nH]ncc1CC1CC2(CNC2)C1 ; Cc1ccc(S(=O)(=O)O)cc1] | 6-[[5-(trifluoromethyl)- 1H-pyrazol-4- yl]methyl]-2- azaspiro[3.3]heptane; 4- methylbenzenesulfonic acid | 4-bromo-5- (trifluoromethyl)- 1H-pyrazole (CAS: 19968- 17-3) | 246.2 [M − TsOH + H]⁺ |
| B.410 | 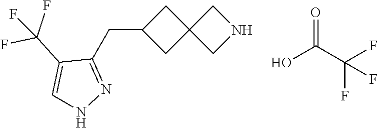 [SMILES: FC(F)(F)c1c[nH]nc1CC1CC2(CNC2)C1 ; O=C(O)C(F)(F)F] | 6-[[4-(trifluoromethyl)- 1H-pyrazol-3- yl]methyl]-2- azaspiro[3.3]heptane; 2,2,2-trifluoroacetic acid | 3-bromo-4- (trifluoromethyl)- 1H-pyrazole (CAS: 1936710- 01-8) | 246.2 [M − TFA + H]⁺ |
| B.413 | 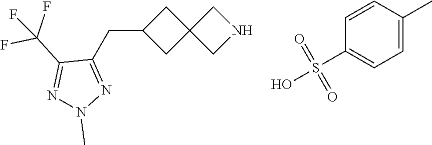 [SMILES: Cn1nc(CC2CC3(CNC3)C2)c(C(F)(F)F)n1 ; Cc1ccc(S(=O)(=O)O)cc1] | 6-[2-methyl-5- (trifluoromethyl)triazol- 4-yl]methyl]-2- azaspiro[3.3]heptane; 4- methylbenzenesulfonic acid | 4-bromo-2- methyl-5- (trifluoromethyl)- 2H-1,2,3- triazole (CAS: 1643359-24-3) | 261.0 [M − TsOH + H]⁺ |
| B.441 | 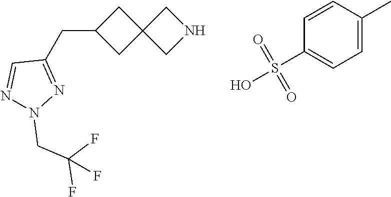 [SMILES: FC(F)(F)Cn1ncc(CC2CC3(CNC3)C2)n1 ; Cc1ccc(S(=O)(=O)O)cc1] | 6-[2-(2,2,2- trifluoroethyl)triazol-4- yl]methyl]-2- azaspiro[3.3]heptane; 4- methylbenzenesulfonic acid | 4,5- dibromo- 2-(2,2,2- trifluoroethyl) triazole (C.11) | 261.0 [M − TsOH + H]⁺ |
| B.442 | 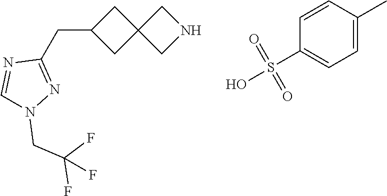 [SMILES: FC(F)(F)Cn1cnc(CC2CC3(CNC3)C2)n1 ; Cc1ccc(S(=O)(=O)O)cc1] | 6-[1-(2,2,2- trifluoroethyl)-1,2,4- triazol-3-yl]methyl]-2- azaspiro[3.3]heptane; 4- methylbenzenesulfonic acid | 3-bromo- 1-(2,2,2- trifluoroethyl)- 1,2,4- triazole (C.12) | 261.3 [M − TFA + H]⁺ |
| B.467 | 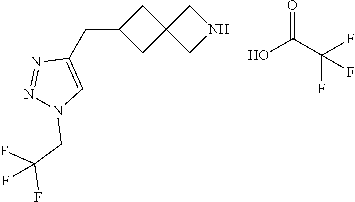 [SMILES: FC(F)(F)Cn1cc(CC2CC3(CNC3)C2)nn1 ; O=C(O)C(F)(F)F] | 6-[[1-(2,2,2- trifluoroethyl)triazol-4- yl]methyl]-2- azaspiro[3.3]heptane; 2,2,2-trifluoroacetic acid | 4-bromo- 1-(2,2,2- trifluoroethyl)- 1H- 1,2,3- triazole (CAS: 1393539- 84-8) | 261.2 [M − TFA + H]⁺ |
| B.492 | 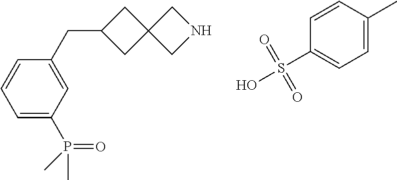 [SMILES: CP(C)(=O)c1cccc(CC2CC3(CNC3)C2)c1 ; Cc1ccc(S(=O)(=O)O)cc1] | 6-[(3- dimethyl- phosphorylphenyl) methyl]-2- azaspiro[3.3]heptane; 4- methylbenzenesulfonic acid | 1-bromo-3- dimethyl- phosphoryl- benzene (CAS: 60398- 84-7) | 264.2 [M − TsOH + H]⁺ |
| B.498 | 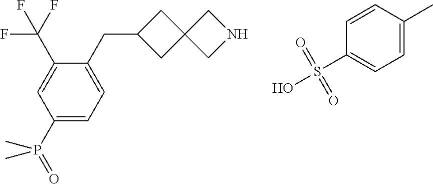 [SMILES: CP(C)(=O)c1ccc(CC2CC3(CNC3)C2)c(C(F)(F)F)c1 ; Cc1ccc(S(=O)(=O)O)cc1] | 6-[[4- dimethylphosphoryl-2- (trifluoromethyl) phenyl] methyl]-2- azaspiro[3.3]heptane; 4- methylbenzenesulfonic acid | 1-bromo- 4- dimethyl- phosphoryl- 2- (trifluoromethyl) benzene (C.14) | 332.0 [M − TsOH + H]⁺ |
| B.499 | 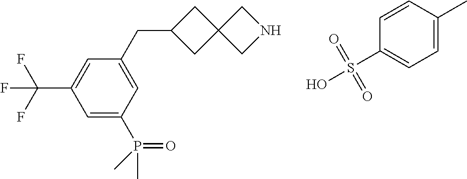 [SMILES: CP(C)(=O)c1cc(CC2CC3(CNC3)C2)cc(C(F)(F)F)c1 ; Cc1ccc(S(=O)(=O)O)cc1] | 6-[[3- dimethylphosphoryl-5- (trifluoromethyl)phenyl methyl]-2- azaspiro[3.3]heptane; 4- methylbenzenesulfonic acid | 1-bromo- 3- dimethyl phosphoryl-5- (trifluoromethyl) benzene (C.15) | 332.2 [M − TsOH + H]⁺ |
| B.500 | 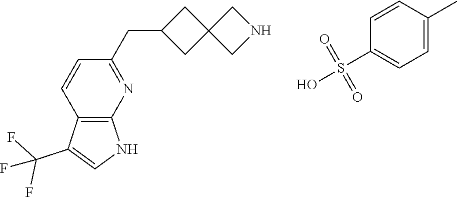 [SMILES: FC(F)(F)c1c[nH]c2nc(CC3CC4(CNC4)C3)ccc12 ; Cc1ccc(S(=O)(=O)O)cc1] | 6-(2- azaspiro[3.3]heptan-6- ylmethyl)-3- (trifluoromethyl)-1H- pyrrolo[2,3-b]pyridine; 4- methylbenzenesulfonic acid | 6-bromo-3- (trifluoro methyl)- 1H- pyrrolo[2,3- b]pyridine (CAS: 1190320- 80-9) | 294.2 [M − TsOH + H]⁺ |
| B.501 | 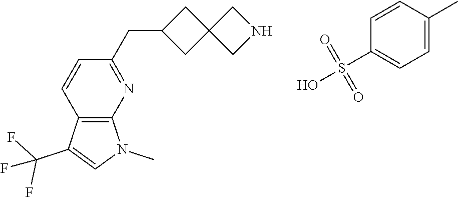 [SMILES: Cn1cc(C(F)(F)F)c2ccc(CC3CC4(CNC4)C3)nc21 ; Cc1ccc(S(=O)(=O)O)cc1] | 6-(2- azaspiro[3.3]heptan-6- ylmethyl)-1-methyl-3- (trifluoromethyl)pyrrolo [2,3-b]pyridine; 4- methylbenzenesulfonic acid | 6-bromo-3- (trifluoromethyl)- 1H- pyrrolo[2,3- b]pyridine (CAS: 1190320-80-9) | 310.2 [M − TsOH + H]⁺ |
| | *via methylation of the precursor | | | |
| | of B.500 after Step c), using NaH, | | | |
| | Mel, followed by deprotection | | | |
| B.503 | 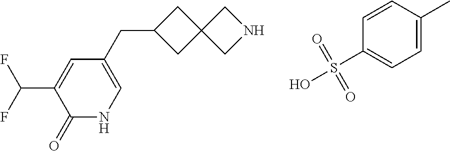 [SMILES: O=c1[nH]cc(CC2CC3(CNC3)C2)cc1C(F)F ; Cc1ccc(S(=O)(=O)O)cc1] | 5-(2- azaspiro[3.3]heptan-6- ylmethyl)-3- (difluoromethyl)-1H- pyridin-2-one; 4- methylbenzenesulfonic acid | 2- benzyloxy-5- bromo-3- (difluoromethyl) pyridine (C.16) | 255.0 [M − TsOH + H]⁺ |
| | *benzyl protecting group also | | | |
| | removed during hydrogenation | | | |
| | step | | | |
| B.505 | 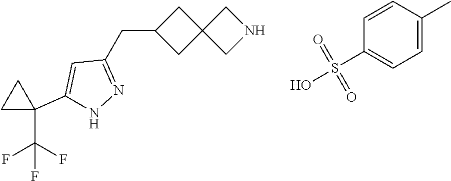 [SMILES: FC(F)(F)C1(c2cc(CC3CC4(CNC4)C3)n[nH]2)CC1 ; Cc1ccc(S(=O)(=O)O)cc1] | 6-[[5-[1- (trifluoromethyl) cyclopropyl]-1- {H}-pyrazol- 3-yl]methyl]-2- azaspiro[3.3]heptane; 4- methylbenzenesulfonic acid | 3-bromo- 5-[1- (trifluoromethyl) cyclopropyl]-1H- pyrazole | 286.1 [M − TsOH + H]⁺ |
| B.506 | 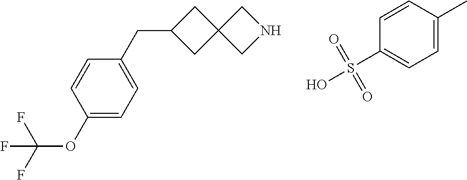 [SMILES: FC(F)(F)Oc1ccc(CC2CC3(CNC3)C2)cc1 ; Cc1ccc(S(=O)(=O)O)cc1] | 6-[[4- (trifluoromethoxy) phenyl]methyl]-2- azaspiro[3.3]heptane; 4- methylbenzenesulfonic acid | 1-bromo-4- (trifluoromethoxy) benzene | 272.2 [M − TsOH + H]⁺ |
| B.507 | 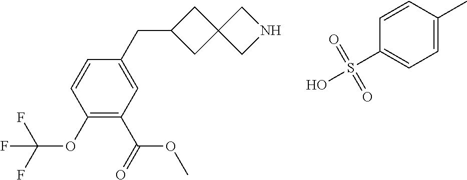 [SMILES: COC(=O)c1cc(CC2CC3(CNC3)C2)ccc1OC(F)(F)F ; Cc1ccc(S(=O)(=O)O)cc1] | methyl 5-(2- azaspiro[3.3]heptan-6- ylmethyl)-2- (trifluoromethoxy) benzoate; 4- methylbenzenesulfonic acid | 5-bromo- 2- (trifluoromethoxy) benzoic acid methyl ester | 330.1 [M − TsOH + H]⁺ |
| B.508 | 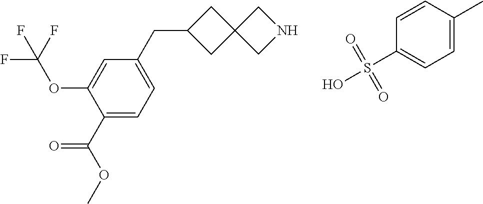 [SMILES: COC(=O)c1ccc(CC2CC3(CNC3)C2)cc1OC(F)(F)F ; Cc1ccc(S(=O)(=O)O)cc1] | methyl 4-(2- azaspiro[3.3]heptan-6- ylmethyl)-2- (trifluoromethoxy) benzoate; 4- methylbenzenesulfonic acid | 4-bromo- 2- (trifluoromethoxy) benzoic acid methyl ester | 330.1 [M − TsOH + H]⁺ |
| B.511 | 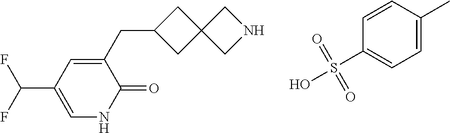 [SMILES: O=c1[nH]cc(C(F)F)cc1CC1CC2(CNC2)C1 ; Cc1ccc(S(=O)(=O)O)cc1] | 3-(2- azaspiro[3.3]heptan-6- ylmethyl)-5- (difluoromethyl)-1H- pyridin-2-one; 4- methylbenzenesulfonic acid; 4- methylbenzenesulfonic acid | 2- benzyloxy-3- bromo-5- (difluoro methyl)pyridine (C.19) | 255.0 [M − TsOH + H]⁺ |
| | *benzyl protecting group also | | | |
| | removed during hydrogenation step | | | |
| B.512 | 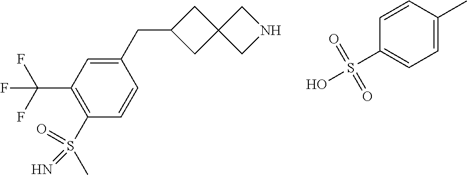 [SMILES: CS(=N)(=O)c1ccc(CC2CC3(CNC3)C2)cc1C(F)(F)F ; Cc1ccc(S(=O)(=O)O)cc1] | [4-(2- azaspiro[3.3]heptan-6- ylmethyl)-2- (trifluoromethyl) phenyl]- imino-methyl-oxo-λ⁶- sulfane; 4- methylbenzenesulfonic acid | [4- bromo-2- (trifluoro methyl) phenyl]- imino- methyl- oxo-λ6- sulfane (C.20) | 333.2 [M − TsOH + H]⁺ |
| B.514 | 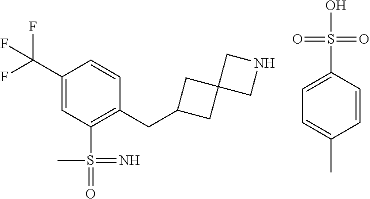 | [2-(2- azaspiro[3.3]heptan-6- ylmethyl)-5- (trifluoromethyl) phenyl]- imino-methyl-oxo-λ⁶- sulfane; 4- methylbenzenesulfonic acid | [2- bromo-5- (trifluoro methyl)phenyl]- imino- methyl- oxo-λ6- sulfane (C.21) | 333.0 [M − TsOH + H]⁺ |
| B.536 | 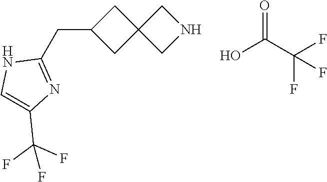 [SMILES: FC(F)(F)c1c[nH]c(CC2CC3(CNC3)C2)n1 ; O=C(O)C(F)(F)F] | 6-[4-(trifluoromethyl)- 1H-imidazol-2- yl]methyl]-2- azaspiro[3.3]heptane; 2,2,2-trifluoroacetic acid | 2-[[2- bromo-4- (trifluoromethyl) imidazol- 1-yl]methoxy] ethyl- trimethyl-silane (C.23) | 246.0 [M − TFA + H]⁺ |
| | (SEM protecting group also | | | |
| | removed during Boc deprotection) | | | |
| B.537 | 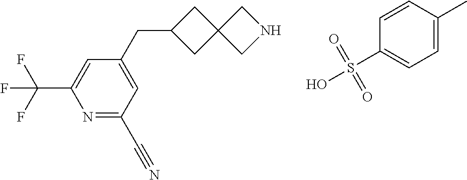 [SMILES: N#Cc1cc(CC2CC3(CNC3)C2)cc(C(F)(F)F)n1 ; Cc1ccc(S(=O)(=O)O)cc1] | 4-(2- azaspiro[3.3]heptan-6- ylmethyl)-6- (trifluoromethyl) pyridine- 2-carbonitrile; 4- methylbenzenesulfonic acid | 4-bromo-6- (trifluoromethyl) picolinonitrile (CAS: 1242968-52-0) | 282.2 [M − TsOH + H]⁺ |
| B.544 | 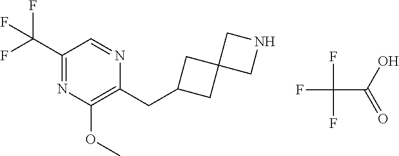 [SMILES: COc1nc(C(F)(F)F)cnc1CC1CC2(CNC2)C1 ; O=C(O)C(F)(F)F] | 6-[[3-methoxy-5- (trifluoromethyl) pyrazin-2-yl]methyl]-2- azaspiro[3.3]heptane; 2,2,2-trifluoroacetic acid | 2-bromo- 3-methoxy-5- (trifluoromethyl) pyrazine (C.25) | 288.2 [M − TFA + H]⁺ |
| B.545 | 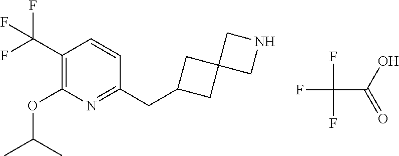 [SMILES: CC(C)Oc1nc(CC2CC3(CNC3)C2)ccc1C(F)(F)F ; O=C(O)C(F)(F)F] | 6-[6-isopropoxy-5- (trifluoromethyl)-2- pyridyl]methyl]-2- azaspiro[3.3]heptane; 2,2,2-trifluoroacetic acid | 6-bromo-2- isopropoxy-3- (trifluoromethyl) pyridine (C.26) | 315.2 [M − TFA + H]⁺ |
| B.556 | 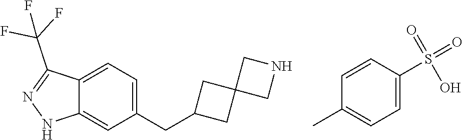 [SMILES: FC(F)(F)c1n[nH]c2cc(CC3CC4(CNC4)C3)ccc12 ; Cc1ccc(S(=O)(=O)O)cc1] | 6-(2- azaspiro[3.3]heptan-6- ylmethyl)-3- (trifluoromethyl)-1H- indazole; 4- methylbenzenesulfonic acid | 6-bromo-3- (trifluoromethyl)- 1H- indazole (CAS: 1374258- 63-5) | 296.1 |
| B.557 | 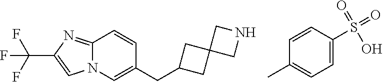 [SMILES: FC(F)(F)c1cn2cc(CC3CC4(CNC4)C3)ccc2n1 ; Cc1ccc(S(=O)(=O)O)cc1] | 6-(2-azaspiro[3.3] heptan-6-ylmethyl)-2- (trifluoromethyl)imidazo [1,2-a]pyridine; 4- methylbenzenesulfonic acid | 6-bromo-2- (trifluoro methyl) imidazo[1,2- a]pyridine (CAS: 150780-40-8) | 296.1 [M − TsOH + H]⁺ |
| B.558 | 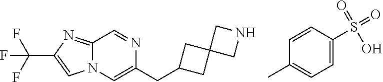 [SMILES: FC(F)(F)c1cn2cc(CC3CC4(CNC4)C3)ncc2n1 ; Cc1ccc(S(=O)(=O)O)cc1] | 6-(2-azaspiro[3.3]heptan- 6-ylmethyl)-2- (trifluoromethyl)imidazo [1,2-a]pyrazine; 4- methylbenzenesulfonic acid | 6-bromo-2- (trifluoro methyl) imidazo[1,2- a]pyrazine (CAS: 1208084-50-7) | 297.4 [M − TsOH + H]⁺ |
| B.559 | 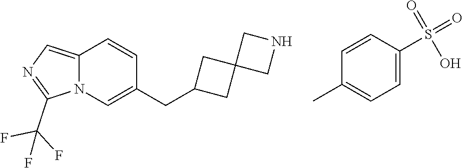 [SMILES: FC(F)(F)c1ncc2ccc(CC3CC4(CNC4)C3)cn12 ; Cc1ccc(S(=O)(=O)O)cc1] | 6-(2- azaspiro[3,3 ]heptan-6- ylmethyl)-3- (trifluoromethyl) imidazo [1,5-a]pyridine; 4- methylbenzenesulfonic acid | 6-bromo-3- (trifluoromethyl) imidazo[1,5- a]pyridine (CAS: 1262619 -64-6) | 296.4 [M − TsOH + H]⁺ |
| B.560 | 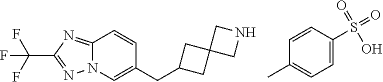 [SMILES: FC(F)(F)c1nc2ccc(CC3CC4(CNC4)C3)cn2n1 ; Cc1ccc(S(=O)(=O)O)cc1] | 6-(2-azaspiro[3.3] heptan-6-ylmethyl)-2- (trifluoromethyl)-[1,2,4] triazolo[1,5-a]pyridine; 4-methylbenzenesulfonic acid | 6-bromo-2- (trifluoromethyl)- [1,2,4]triazolo [1,5- a]pyridine (CAS: 1159812-99-3) | 297.1 [M − TsOH + H]⁺ |
| B.561 | 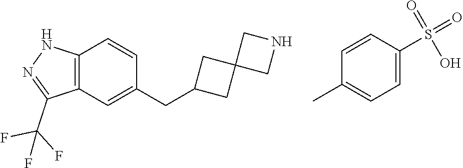 [SMILES: FC(F)(F)c1n[nH]c2ccc(CC3CC4(CNC4)C3)cc12 ; Cc1ccc(S(=O)(=O)O)cc1] | 5-(2- azaspiro[3.3]heptan-6- ylmethyl)-3- (trifluoromethyl)-1H- indazole;4- methylbenzenesulfonic acid | 5-bromo-3- (trifluoromethyl)- 1H- indazole (CAS: 57631-11-5) | 296.1 [M − TsOH + H]⁺ |
| B.562 | 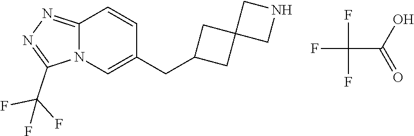 [SMILES: FC(F)(F)c1nnc2ccc(CC3CC4(CNC4)C3)cn12 ; O=C(O)C(F)(F)F] | 6-(2- azaspiro[3.3]heptan-6- ylmethyl)-3- (trifluoromethyl)- [1,2,4]triazolo[4,3- a]pyridine; trifluoroacetic acid | 6-bromo-3- (trifluoromethyl)- [1,2,4]triazolo [4,3-a]pyridine (CAS: 1166819- 53-9) | 297.2 [M − TFA + H]⁺ |
| B.563 | 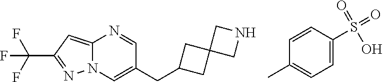 [SMILES: FC(F)(F)c1cc2ncc(CC3CC4(CNC4)C3)cn2n1 ; Cc1ccc(S(=O)(=O)O)cc1] | 6-(2-azaspiro[3.3] heptan-6-ylmethyl)-2- (trifluoromethyl)pyrazolo [1,5-a]pyrimidine; 4- methylbenzenesulfonic acid | 6-bromo-2- (trifluoromethyl) pyrazolo[1,5-a] pyrimidine (CAS: 1379358-08-3) | [M − TsOH + H]⁺ |

### Example B.27

### 7-[6-(trifluoromethyl)pyridazin 3-yl]oxy-2-azaspiro[3.5]nonane; 4-methylbenzenesulfonic Acid

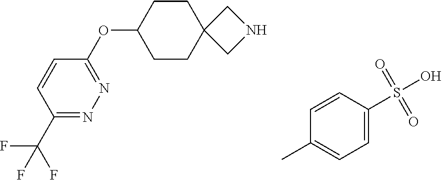 [SMILES: FC(F)(F)c1ccc(OC2CCC3(CC2)CNC3)nn1 ; Cc1ccc(S(=O)(=O)O)cc1]

To a suspension of 7-[6-(trifluoromethyl)pyridazin-3-yl]oxy-2-azaspiro[3.5]nonane-2-carboxylic acid tert-butyl ester (350 mg, 0.903 mmol) in isopropyl acetate (4 mL) and was added p-toluenesulfonic acid monohydrate (258 mg, 1.36 mmol). The mixture was stirred at 100° C. for 6 h. The reaction mixture was concentrated in vacuo. Et₂O was added, and the mixture filtered through sintered glass. The white solid was washed with Et₂O (2×) and dried in vacuo to afford the title compound (0.420 g, 88%) as a white solid. MS (ESI): m/z=288.0 [M-TsOH+H]⁺

### Step a) 7-[6-(trifluoromethyl)pyridazin-3 yl]oxy-2-azaspiro[3.5]nonane-2-carboxylic Acid Tert-Butyl Ester

To a solution of 7-hydroxy-2-azaspiro[3.5]nonane-2-carboxylic acid tert-butyl ester (CAS: 1363383-18-9) (350 mg, 1.45 mmol) and potassium t-butoxide (195 mg, 1.74 mmol) in N,N-dimethylformamide (3.5 mL) was added 3-fluoro-6-(trifluoromethyl)pyridazine (248 mg, 1.49 mmol). The mixture was stirred at 80° C. for 15 h. The reaction mixture was poured into EtOAc and washed with water and brine. The organic layer was dried over Na₂SO₄ and concentrated in vacuo. The crude material was purified by flash chromatography (eluting with 0% to 40% EtOAc in heptane) to afford the title compound (353 mg, 59.69%) as white solid. MS (ESI): m/z=332.1 [M-tBu+H]⁺

In analogy to Example B.27, the following building blocks were generated using the relevant (hetero)aryl halide and hydroxy-spirocyclic building blocks. In some cases alternative solvents e.g. DMSO in Step a) were used.

| | | | | MS, |
|---|---|---|---|---|
| | | | Building | ESI: |
| Ex. | Structure | Systematic Name | Blocks | m/z |
| B.43 | 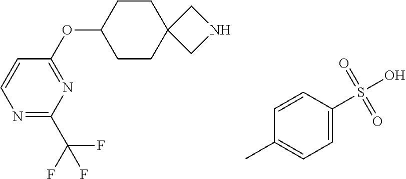 [SMILES: FC(F)(F)c1nccc(OC2CCC3(CC2)CNC3)n1 ; Cc1ccc(S(=O)(=O)O)cc1] | 7-[2- (trifluoromethyl) pyrimidin- 4-ylJoxy-2- azaspiro[3.5]nonane; 4- methylbenzenesulfonic acid | 4-chloro-2- (trifluoromethyl) pyrimidine, 7-hydroxy-2- azaspiro[3.5] nonane-2- carboxylic acid tert-butyl ester | 288.1 [M − TsOH + H]⁺ |
| B.44 | 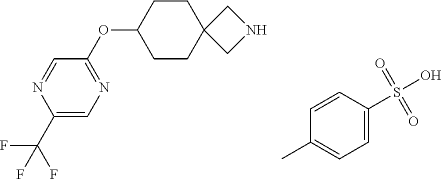 [SMILES: FC(F)(F)c1cnc(OC2CCC3(CC2)CNC3)cn1 ; Cc1ccc(S(=O)(=O)O)cc1] | 7-[5-(trifluoromethyl) pyrazin- 2-yl]oxy-2- azaspiro[3.5]nonane; 4- methylbenzenesulfonic acid | fluoro-5- (trifluoromethyl) pyrazine, 7- hydroxy-2- azaspiro[3.5] nonane-2- carboxylic acid tert-butyl ester | 288.1 [M − TsOH + H]⁺ |
| B.45 | 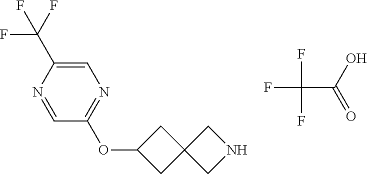 [SMILES: FC(F)(F)c1cnc(OC2CC3(CNC3)C2)cn1 ; O=C(O)C(F)(F)F] | 6-[5- (trifluoromethyl)pyrazin- 2-yl]oxy-2- azaspiro[3.3]heptane; trifluoroacetic acid | 2-chloro-5- (trifluoromethyl) pyrazine, 6- hydroxy-2- azaspiro[3.3] heptane-2- carboxylic acid tert-butyl ester (CAS: 1147557-97-8) | 260.1 [M − TsOH + H]⁺ |
| B.46 | 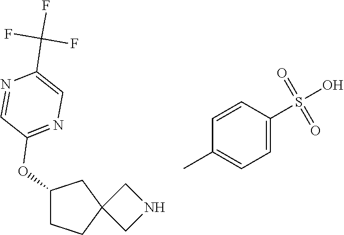 [SMILES: FC(F)(F)c1cnc(O[C@H]2CCC3(CNC3)C2)cn1 ; Cc1ccc(S(=O)(=O)O)cc1] | (6S)-6-[5- (trifluoromethyl)pyrazin- 2-yl]oxy-2- azaspiro[3.4]octane; 4- methylbenzenesulfonic acid | 2-fluoro-5- (trifluoromethyl) pyrazine, (6S)-6- hydroxy-2- azaspiro[3.4] octane-2- carboxylic acid tert-butyl ester (CAS: 2376903-72-7) | 274.0 [M − TsOH + H]⁺ |
| B.48 | 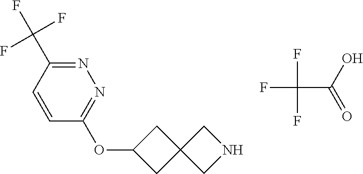 [SMILES: FC(F)(F)c1ccc(OC2CC3(CNC3)C2)nn1 ; O=C(O)C(F)(F)F] | 6-[6- (trifluoromethyl) pyridazin- 3-yl]oxy-2- azaspiro[3.3]heptane; trifluoroacetic acid | 3-chloro-6- (trifluoromethyl) pyridazine; 6-hydroxy-2- azaspiro[3.3] heptane-2- carboxylic acid tert-butyl ester (CAS: 1147557-97-8) | 260.1 [M − TsOH + H]⁺ |
| B.84 | 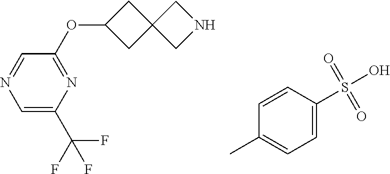 [SMILES: FC(F)(F)c1cncc(OC2CC3(CNC3)C2)n1 ; Cc1ccc(S(=O)(=O)O)cc1] | 6-[6- (trifluoromethyl)pyrazin- 2-yl]oxy-2- azaspiro[3.3]heptane; 4- methylbenzenesulfonic acid | 2-chloro-6- (trifluoromethyl) pyrazine (CAS: 61655- 69-4); 6- hydroxy-2- azaspiro[3.3] heptane-2- carboxylic acid tert-butyl ester | 260.1 [M − TsOH + H]⁺ |
| B.85 | 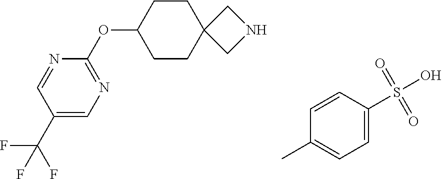 [SMILES: FC(F)(F)c1cnc(OC2CCC3(CC2)CNC3)nc1 ; Cc1ccc(S(=O)(=O)O)cc1] | 7-[5- (trifluoromethyl)pyrimidin- 2-yl]oxy-2- azaspiro[3.5]nonane; 4- methylbenzenesulfonic acid | 2-chloro-5- (trifluoromethyl) pyrimidine, 7-hydroxy-2- azaspiro[3.5] nonane-2- carboxylic acid tert-butyl ester | 288.1 [M − TsOH + H]⁺ |
| B.97 | 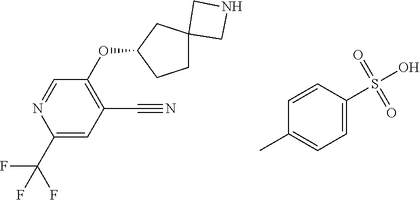 [SMILES: N#Cc1cc(C(F)(F)F)ncc1O[C@H]1CCC2(CNC2)C1 ; Cc1ccc(S(=O)(=O)O)cc1] | 5-[[(6S)-2- azaspiro[3.4]octan-6- yl]oxy]-2- (trifluoromethyl)pyridine- 4-carbonitrile; 4- methylbenzenesulfonic acid | 5-bromo-2- (trifluoromethyl) isonicotinonitrile (CAS: 1070892-04-4) and (6S)-6- hydroxy-2- azaspiro[3.4] octane-2- carboxylic acid tert-butyl ester (CAS: 2376903-72-7) | 298.2 [M − TsOH + H]⁺ |
| B.98 | 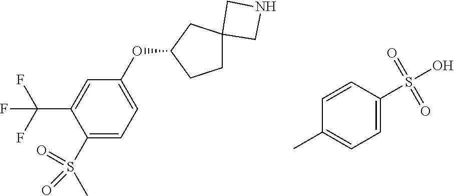 [SMILES: CS(=O)(=O)c1ccc(O[C@H]2CCC3(CNC3)C2)cc1C(F)(F)F ; Cc1ccc(S(=O)(=O)O)cc1] | (6S)-6-[4- methylsulfonyl-3- (trifluoromethyl)phenoxy]- 2-azaspiro[3.4]octane; 4-methylbenzenesulfonic acid | 4-bromo-1- mesyl-2- (trifluoromethyl) benzene and (6S)-6- hydroxy-2- azaspiro[3.4] octane-2- carboxylic acid tert-butyl ester (CAS: 2376903-72-7) and | 350.2 [M − TsOH + H]⁺ |
| B.230 | 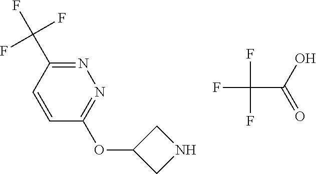 [SMILES: FC(F)(F)c1ccc(OC2CNC2)nn1 ; O=C(O)C(F)(F)F] | 3-(azetidin-3-yloxy)-6- (trifluoromethyl)pyridazine; 2,2,2-trifluoroacetic acid | 3-chloro-6- (trifluoromethyl) pyridazine (CAS RN: 258506-68-2) and tert-Butyl 3- hydroxyazetidine- 1-carboxylate (CAS RN: 141699-55-0) | 220.1 [M + H]⁺ |
| | In Step a), NaH was used | | | |
| | instead of KOtBu | | | |
| B.260 | 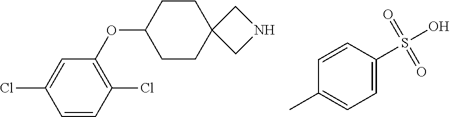 [SMILES: Clc1ccc(Cl)c(OC2CCC3(CC2)CNC3)c1 ; Cc1ccc(S(=O)(=O)O)cc1] | 7-(2,5-dichlorophenoxy)- 2-azaspiro[3.5]nonane;4- methylbenzenesulfonic acid | 1,4-dichloro-2- methylsulfonyl benzene (CAS RN: 66640-63- 9), 7-hydroxy-2- azaspiro[3.5] nonane-2- carboxylic acid | 286.2 [M + H]⁺ |
| | | | tert-butyl ester | |
| | | | (CAS RN: | |
| | | | 1363383-18-9) | |
| B.264 | 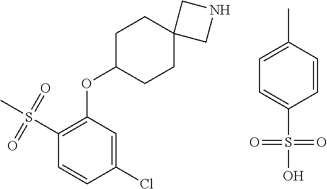 [SMILES: CS(=O)(=O)c1ccc(Cl)cc1OC1CCC2(CC1)CNC2 ; Cc1ccc(S(=O)(=O)O)cc1] | 7-(5-chloro-2- methylsulfonyl- phenoxy)-2- azaspiro[3.5]nonane;4- methylbenzenesulfonic acid | 4-chloro-2- fluoro-1- mesyl-benzene (CAS RN: 1261284-27- 8), 7-hydroxy-2- azaspiro[3.5] nonane-2- carboxylic acid tert-butyl ester (CAS RN: 1363383-18-9) | 330.1 [M + H]⁺ |
| B.269 | 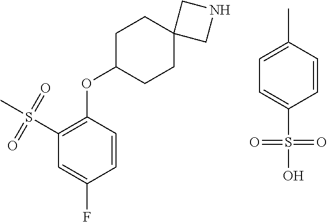 [SMILES: CS(=O)(=O)c1cc(F)ccc1OC1CCC2(CC1)CNC2 ; Cc1ccc(S(=O)(=O)O)cc1] | 7-(4-fluoro-2- methylsulfonyl- phenoxy)-2- azaspiro[3.5]nonane;4- methylbenzenesulfonic acid | 1,4-difluoro-2- mesyl-benzene (CAS RN: 61655-69-4), 7-hydroxy-2- azaspiro[3.5] nonane-2- carboxylic acid tert-butyl ester (CAS RN: 1363383-18-9) | 314.1 [M + H]⁺ |
| B.332 | 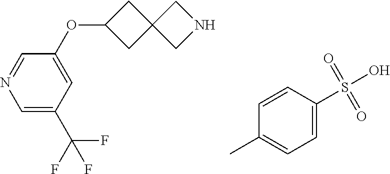 [SMILES: FC(F)(F)c1cncc(OC2CC3(CNC3)C2)c1 ; Cc1ccc(S(=O)(=O)O)cc1] | 6-[[5-(trifluoromethyl)-3- pyridyl]oxy]-2- azaspiro[3.3]heptane; 4- methylbenzenesulfonic acid | 3-fluoro-5- (trifluoromethyl) pyridine; 6- hydroxy-2- azaspiro[3.3] heptane-2- carboxylic acid tert-butyl ester | 260.1 [M − TsOH + H]⁺ |
| B.333 | 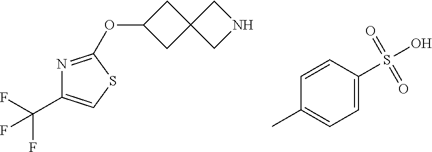 [SMILES: FC(F)(F)c1csc(OC2CC3(CNC3)C2)n1 ; Cc1ccc(S(=O)(=O)O)cc1] | 2-(2-azaspiro[3.3]heptan- 6-yloxy)-4- (trifluoromethyl)thiazole; 4-methylbenzenesulfonic acid | 2-bromo-4- (trifluoromethyl) thiazole; 6- hydroxy-2- azaspiro[3.3] heptane-2- carboxylic acid | 265.0 [M − TsOH + H]⁺ |
| B.340 | 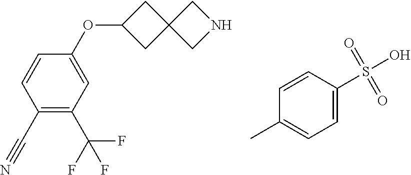 [SMILES: N#Cc1ccc(OC2CC3(CNC3)C2)cc1C(F)(F)F ; Cc1ccc(S(=O)(=O)O)cc1] | 4-(2-azaspiro[3.3]heptan- 6-yloxy)-2- (trifluoromethyl) benzonitrile;4- methylbenzenesulfonic acid | 4-fluoro-2- (trifluoromethyl) benzonitrile; 6-hydroxy-2- azaspiro[3.3] heptane-2- carboxylic acid tert-butyl ester | 283.0 [M − TsOH + H]⁺ |
| B.341 | 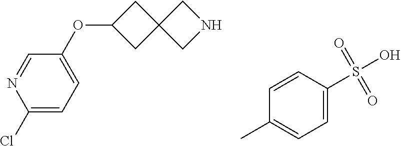 | 6-[(5-chloro-2- pyridyl)oxy]-2- azaspiro[3.3]heptane; 4- methylbenzenesulfonic acid | 5-chloro-2- fluoropyridine; 6-hydroxy-2- azaspiro[3.3] heptane-2- carboxylic acid tert-butyl ester | 225.0 [M − TsOH + H]⁺ |
| B.342 | 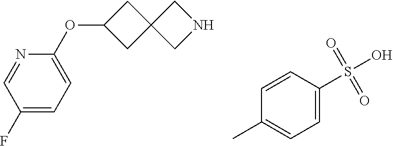 | 6-[(5-fluoro-2- pyridyl)oxy]-2- azaspiro[3.3]heptane; 4- methylbenzenesulfonic acid | 2,5- difluoropyridine; 6-hydroxy- 2- azaspiro[3.3] heptane-2- carboxylic acid tert-butyl ester | 209.1 [M − TsOH + H]⁺ |
| B.343 | 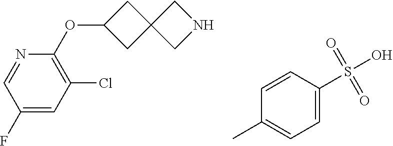 [SMILES: Fc1cnc(OC2CC3(CNC3)C2)c(Cl)c1 ; Cc1ccc(S(=O)(=O)O)cc1] | 6-[(3-chloro-5-fluoro-2- pyridyl)oxy]-2- azaspiro[3.3]heptane; 4- methylbenzenesulfonic acid | 5-chloro-2- fluoropyridine; 6-hydroxy-2- azaspiro[3.3] heptane-2- carboxylic acid tert-butyl ester | 243.0 [M − TsOH + H]⁺ |
| B.527 | 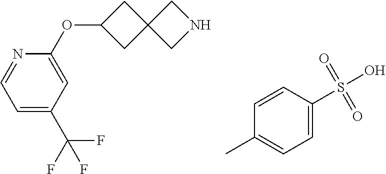 [SMILES: FC(F)(F)c1ccnc(OC2CC3(CNC3)C2)c1 ; Cc1ccc(S(=O)(=O)O)cc1] | 6-[4-(trifluoromethyl)-2- pyridyl]oxy]-2- azaspiro[3.3]heptane; 4- methylbenzenesulfonic acid | 2-fluoro-4- (trifluoromethyl) pyridine; 6- hydroxy-2- azaspiro[3.3] heptane-2- carboxylic acid tert-butyl ester | 259.0 [M − TsOH + H]⁺ |
| B.546 | 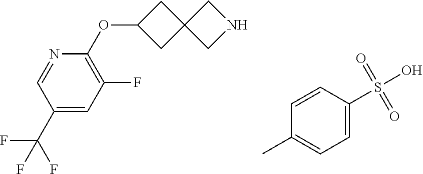 [SMILES: Fc1cc(C(F)(F)F)cnc1OC1CC2(CNC2)C1 ; Cc1ccc(S(=O)(=O)O)cc1] | 6-[[3-fluoro-5- (trifluoromethyl)-2- pyridyl]oxy]-2- azaspiro[3.3]heptane; 4- methylbenzenesulfonic acid | 2,3-difluoro-5- (trifluoromethyl) pyridine (CAS: 89402- 42-6); 6- hydroxy-2- azaspiro[3.3] heptane-2- carboxylic acid tert-butyl ester | 277.0 [M − TsOH + H]⁺ |
| M.20 | 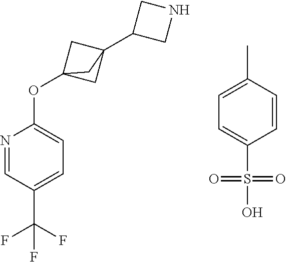 [SMILES: FC(F)(F)c1ccc(OC23CC(C4CNC4)(C2)C3)nc1 ; Cc1ccc(S(=O)(=O)O)cc1] | 2-[[3-(azetidin-3-yl)-1- bicyclo[1.1.1]pentanyl] oxy]-5- (trifluoromethyl)pyridine; 4- methylbenzenesulfonic acid | tert-butyl 3-(3- hydroxy-1- bicyclo[1.1.1] pentanyl)azetidine- 1- carboxylate (CAS RN: 2490406-07- 8), 2-fluoro-5- (trifluoromethyl) pyridine (CAS RN: 69045-82-5) | 285.2 [M + H]⁺ |

### Example B.38

### 6-[5-(trifluoromethyl)pyrazin-2-yl]-2-azaspiro[3.3]heptane; 4-methylbenzenesulfonic Acid

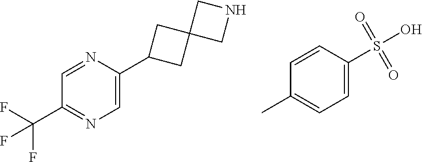 [SMILES: FC(F)(F)c1cnc(C2CC3(CNC3)C2)cn1 ; Cc1ccc(S(=O)(=O)O)cc1]

A solution of tert-butyl 6-[2-(trifluoromethyl)pyrimidin-5-yl)-2-azaspiro[3.3]heptane-2-carboxylate (1100 mg, 3.2 mmol) and p-toluenesulfonic acid (662 mg, 3.84 mmol) in EtOAc (10 mL) was stirred at 80° C. for 16 h. The mixture was concentrated under reduced pressure and deionized water was added and the mixture lyophilized to give the title compound (1300 mg, 3.13 mmol, 94% yield) as a white solid. MS (ESI): m/z=244.0 [M-TsOH+H]⁺

### Step a) Tert-Butyl 6-[2-(trifluoromethyl)pyrimidin-5-yl]-2-azaspiro[3.3]heptane-2-carboxylate

To a solution of tert-butyl 6-iodo-2-azaspiro[3.3]heptane-2-carboxylate (CAS: 2059140-61-1) (3130 mg, 9.69 mmol), 5-bromo-2-(trifluoromethyl)pyrimidine (1100 mg, 4.85 mmol), Ir[dF(CF₃)ppy]₂(dtbpy)(PF₆) (54.3 mg, 0.050 mmol), NiCl₂·dtbbpy (9.64 mg, 0.020 mmol), TTMSS (1205 mg, 4.85 mmol), Na₂CO₃ (1027 mg, 9.69 mmol) in DME (20 mL). The vial was sealed and placed under nitrogen was added. The reaction was stirred and irradiated with a 34 W blue LED lamp (7 cm away), with cooling fan to keep the reaction temperature at 25° C. for 14 h. The residue was purified by flash silica gel chromatography (eluent of 0-30% Ethyl acetate/Petroleum ether gradient) to give a residue which was purified by reversed-phase HPLC (0.1% FA condition) to give the title compound (1.1 g, 3.2 mmol, 66% yield) as a white solid. MS (ESI): m/z=288.1 [M-tBu+H]⁺

In analogy to Example B.38, the following building blocks were generated using the relevant (hetero)aryl bromide building block for the photochemical coupling in Step a. In some cases, alternative salts (e.g. trifluoroacetate, ditosylate, hydrochloride) were also used.

| | | | | MS, |
|---|---|---|---|---|
| | | | Building | ESI: |
| Ex. | Structure | Systematic Name | Blocks | m/z |
| B.49 | 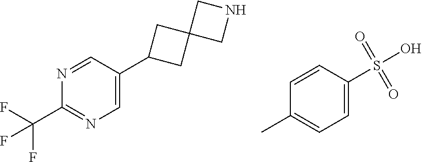 [SMILES: FC(F)(F)c1ncc(C2CC3(CNC3)C2)cn1 ; Cc1ccc(S(=O)(=O)O)cc1] | 6-[2- (trifluoromethyl) pyrimidin-5-yl]-2- azaspiro[3.3]heptane; 4- methylbenzenesulfonic acid | 5-bromo- 2- (trifluoro methyl) pyrimidine | 244.2 [M TsOH + H]⁺ |
| B.324 | 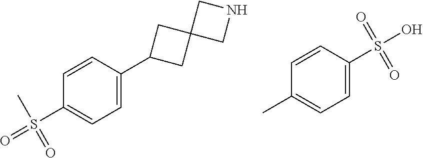 [SMILES: CS(=O)(=O)c1ccc(C2CC3(CNC3)C2)cc1 ; Cc1ccc(S(=O)(=O)O)cc1] | 6-(4- methylsulfonylphenyl)- 2-azaspiro[3.3]heptane; 4- methylbenzenesulfonic acid | 4- bromophenyl methyl sulfone | 252.1 [M − TsOH + H]⁺ |
| B.328 | 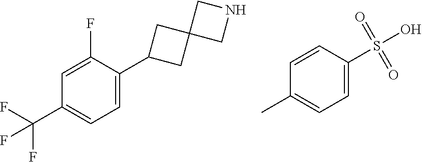 [SMILES: Fc1cc(C(F)(F)F)ccc1C1CC2(CNC2)C1 ; Cc1ccc(S(=O)(=O)O)cc1] | 6-[2-fluoro-4- (trifluoromethyl)phenyl 1-2- azaspiro[3.3]heptane; 4- methylbenzenesulfonic acid | 4-bromo- 3- fluorobenzo- trifluoride | 260.0 [M − TsOH + H]⁺ |
| B.370 | 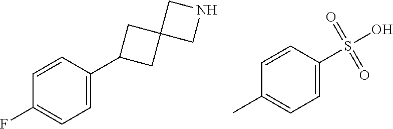 | 6-(4-fluorophenyl)-2- azaspiro[3.3]heptane; 4- methylbenzenesulfonic acid | 4- bromofluoro- benzene | 192.1 [M − TsOH + H]⁺ |
| B.371 | 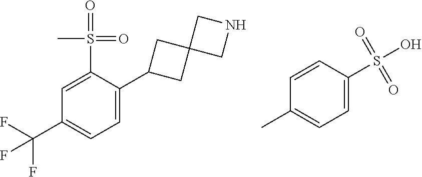 [SMILES: CS(=O)(=O)c1cc(C(F)(F)F)ccc1C1CC2(CNC2)C1 ; Cc1ccc(S(=O)(=O)O)cc1] | 6-[2-methylsulfonyl-4- (trifluoromethyl) phenyl]-2- azaspiro[3.3]heptane; 4- methylbenzenesulfonic acid | 1-bromo- 2- methylsulfonyl- 4- (trifluoro methyl) benzene (CAS: 1215205- 98-3) | 192.1 [M − TsOH + H]⁺ |
| B.547 | 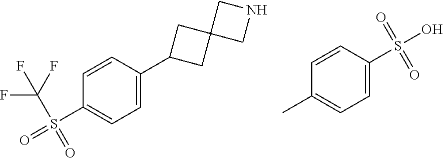 [SMILES: O=S(=O)(c1ccc(C2CC3(CNC3)C2)cc1)C(F)(F)F ; Cc1ccc(S(=O)(=O)O)cc1] | 6-[4- (trifluoromethylsulfonyl) phenyl]-2- azaspiro[3.3]heptane; 4- methylbenzenesulfonic acid | 1-bromo- 4- ((trifluoromethyl) sulfonyl) benzene (CAS: 312-20- 9) | 306.2 [M − TsOH + H]⁺ |

### Example B.325

### 6-(5-fluoro-3-pyridyl)-2-azaspiro[3.3]heptane; 4-methylbenzenesulfonic Acid

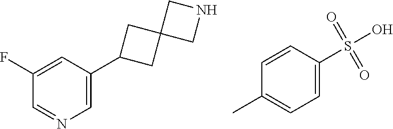

The solution of tert-butyl 6-(5-fluoro-3-pyridyl)-2-azaspiro[3.3]heptane-2-carboxylate (4.0 g, 13.68 mmol) and p-toluenesulfonic acid monohydrate (6.51 g, 34.21 mmol) in EtOAc (150 mL) was stirred at 25° C. for 18 h. Then RM was evaporated and obtained residue (as an oil) was stirred with TBME (150 mL) for 6 h. The obtained precipitate was filtered, washed with TBME and dried to give the title compound (5.66 g, 10.6 mmol, 73.2% yield) as white solid. MS (ESI): m/z=193.2 [M-TsOH+H]⁺

### Step a) Tert-Butyl 6-(5-fluoro-3-pyridyl)-2-azaspiro[3.3]heptane-2-carboxylate

To the stirred mixture of 5-fluoropyridine-3-boronic acid (CAS: 872041-86-6) (6.1 g, 43.3 mmol), (1S,2S)-2-aminocyclohexanol (249 mg, 2.17 mmol), Nickel(II) iodide (677 mg, 2.17 mmol) in iPrOH (140 mL) under Argon atmosphere, 2 M sodium bis(trimethylsilyl)amide solution in THE (21.7 mL, 43.3 mmol) was added via syringe at RT. Then RM was stirred for 10 min at ambient temperature, before tert-butyl 6-iodo-2-azaspiro[3.3]heptane-2-carboxylate (CAS: 2059140-61-1) (7.0 g, 21.7 mmol) was added. The reaction mixture was refluxed for 4 h and then stirred overnight at RT. The obtained mixture was filtered through SiO₂ and filter-cake washed with IPA. The filtarate was evaporated and residue was partioned between TBME and water. The organic layer was dried over Na₂SO₄ and evaporated in vacuum. The obtained crude product was purified with flash column chromatography to give the title compounds (3.95 g, 13.5 mmol, 59.3% yield) as white solid. MS (ESI): m/z=293.2 [M-TsOH+H]⁺

### Example B.29

### N-(2-azaspiro[3.3]heptan-6-ylmethyl)-1-(trifluoromethyl)cyclopropanamine; 4-methylbenzenesulfonic Acid

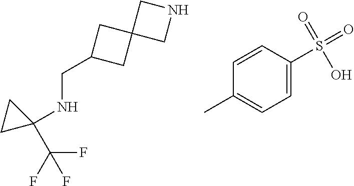 [SMILES: FC(F)(F)C1(NCC2CC3(CNC3)C2)CC1 ; Cc1ccc(S(=O)(=O)O)cc1]

p-toluenesulfonic acid monohydrate (1024 mg, 5.38 mmol) was added to a stirred solution of tert-butyl 6-[[[1-(trifluoromethyl)cyclopropyl]amino]methyl]-2-azaspiro[3.3]heptane-2-carboxylate (600 mg, 1.79 mmol) in acetonitrile (20 mL). The reaction mixture was stirred for 16 h. The solvent was evaporated under reduced pressure and the residue was triturated with MTBE to give the title compound (668 mg, 1.15 mmol, 64% yield) as a white solid. MS (ESI): m/z=235.2 [M-TsOH+H]⁺

### Step a) Tert-Butyl 6-[[1-(trifluoromethyl)cyclopropyl]carbamoyl]-2-azaspiro[3.3]heptane-2-carboxylate

To a stirred solution of 2-tert-butoxycarbonyl-2-azaspiro[3.3]heptane-6-carboxylic acid (CAS: 1211526-53-2) (2.0 g, 8.29 mmol) and 1-(trifluoromethyl)cyclopropanamine hydrochloride (CAS: 112738-67-7) (1340 mg, 8.29 mmol) in DMF (5 mL) were added O-(7-azabenzotriazol-1-yl)-N,N,N′,N′-tetramethyluronium hexafluorophosphate (3782 mg, 9.95 mmol) and N,N-diisopropylethylamine (5.05 mL, 29.0 mmol). The mixture was stirred overnight at room temp. and then poured onto water and extracted with EtOAc (2×50 mL). The combined organic fractions were washed three times with water, dried under anhydrous sodium sulfate, and the solvent was removed under vacuum. The residue was triturated with 25 ml of MTBE to give the title compound (1.7 g, 4.88 mmol, 59% yield) as white solid. MS (ESI): m/z=347.2 [M−H]⁻

### Step b) Tert-Butyl 6-[[[1-(trifluoromethyl)cyclopropyl]amino]methyl]-2-azaspiro[3.3]heptane-2-carboxylate

Tert-butyl 6-[[1-(trifluoromethyl)cyclopropyl]carbamoyl]-2-azaspiro[3.3]heptane-2-carboxylate (1.1 g, 3.16 mmol) was dissolved in THE (30 mL). Borane-methyl sulfide complex (0.48 g, 6.32 mmol) was added at 0 C. The reaction mixture was stirred at reflux for 6 h and then cooled to 0 C and quenched with the drop-wise addition of methanol (5 mL) and then concentrated in vacuo. The residue was diluted with brine and then extracted with EtOAc (3 times). The organic layers were combined, dried over anhydrous sodium sulfate, filtered and concentrated to afford the title compound (600 mg, 1.79 mmol, 57% yield) as colorless oil. MS (ESI): m/z=279.0 [M-tBu+H]⁺

### Example B.30

### N-(2-azaspiro[3.3]heptan-6-yl)-3-(trifluoromethyl)benzenesulfonamide; trifluoroacetic Acid

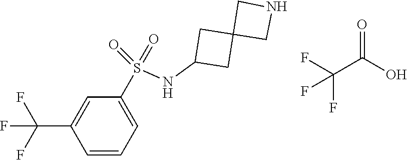

To a solution of 6-[[3-(trifluoromethyl)phenyl]sulfonylamino]-2-azaspiro[3.3]heptane-2-carboxylic acid tert-butyl ester (1245 mg, 2.96 mmol) in dichloromethane (8 mL) was added TFA (3.38 g, 2.28 mL, 29.6 mmol) and the reaction mixture was then stirred at room temp for 18 h. Volatiles were removed in vacuo to yield 1910 mg of the crude title compound, purity of roughly 65% and major contaminant excess of TFA, which was used without further purification. MS (ESI): m/z=321.1 [M-TFA+H]⁺

### Step a) 6-[[3-(trifluoromethyl)phenyl]sulfonylamino]-2-azaspiro[3.3]heptane-2-carboxylic Acid Tert-Butyl Ester

To a solution of 6-amino-2-azaspiro[3.3]heptane-2-carboxylic acid tert-butyl ester (750 mg, 3.53 mmol) in dichloromethane (15 mL) cooled down to 0° C. was added DIPEA (685 mg, 926 μL, 5.3 mmol) and 3-(trifluoromethyl)benzenesulfonyl chloride (907 mg, 3.71 mmol) after which the reaction mixture was stirred at 0° C. for 30 min and at r.t for 1 h. The reaction mixture was poured into a separating funnel containing dichloromethane and aq. sol. Na₂CO₃ 1M. The organic phase was collected and the aqueous phase was back-extracted with dichloromethane. The combined organic phases were dried over sodium sulfate and evaporated down to dryness. The crude material was purified by flash chromatography with an eluent mixture of heptane and ethyl acetate (10% to 90%) to yield 775 mg of the title compound. MS (ESI): m/z=365.1 [M-tBu+H]⁺

In analogy to Example B.30, the following building blocks were generated using the relevant sulfonyl chloride building block. For examples B.141-B. 144, 3-aminoazetidine-1-carboxylic acid tert-butyl ester was used in place of 6-amino-2-azaspiro[3.3]heptane-2-carboxylic acid tert-butyl ester. In examples B. 146-B.147, 3-(aminomethyl)azetidine-1-carboxylic acid tert-butyl ester was used in place of 6-amino-2-azaspiro[3.3]heptane-2-carboxylic acid tert-butyl ester. In Example B.175, an ethoxycarbamate protecting group was used instead of Boc, and was removed by alkaline hydrolysis. In Examples B.176-B.178, 4-(aminomethyl)piperidine-1-carboxylic acid tert-butyl ester was used in place of 6-amino-2-azaspiro[3.3]heptane-2-carboxylic acid tert-butyl ester. In some cases, alternative salts (e.g. trifluoroacetate, tosylate, ditosylate, hydrochloride) were also used.

For B. 178, the free base was isolated after deprotection with HCl and extraction from aqueous 33% NaOH solution in the final step.

| | | | | MS, |
|---|---|---|---|---|
| | | | Building | ESI: |
| Ex. | Structure | Systematic Name | Blocks | m/z |
| B.141 | 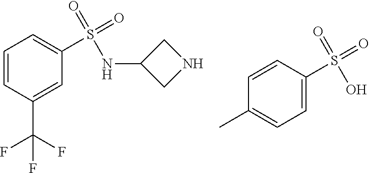 [SMILES: O=S(=O)(NC1CNC1)c1cccc(C(F)(F)F)c1 ; Cc1ccc(S(=O)(=O)O)cc1] | N-(azetidin-3-yl)-3- (trifluoromethyl) benzenesulfonamide; 4- methylbenzenesulfonic acid | 3- (trifluoro methyl) benzene- sulfonyl chloride | 281.0 [M − TsOH + H]⁺ |
| B.142 | 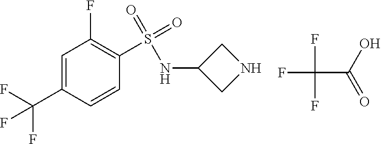 | N-(azetidin-3-yl)-2- fluoro-4- (trifluoromethyl) benzenesulfonamide; trifluoroacetic acid | 2-fluoro- 4- (trifluoromethyl) benzenesulfonyl chloride | 299.1 [M − TsOH + H]⁺ |
| B.143 | 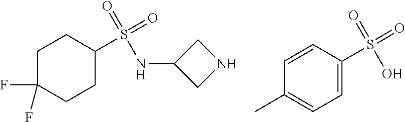 [SMILES: O=S(=O)(NC1CNC1)C1CCC(F)(F)CC1 ; Cc1ccc(S(=O)(=O)O)cc1] | N-(azetidin-3-yl)-4,4- difluoro- cyclohexanesulfonamide; 4-methylbenzenesulfonic acid | 4,4- difluorocyclo- hexanesulfonyl chloride | 255.0 [M − TsOH + H]⁺ |
| B.144 | 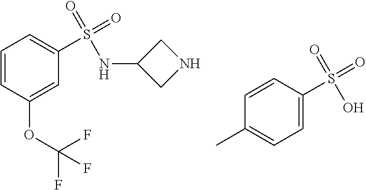 [SMILES: O=S(=O)(NC1CNC1)c1cccc(OC(F)(F)F)c1 ; Cc1ccc(S(=O)(=O)O)cc1] | N-(azetidin-3-yl)-3- (trifluoromethoxy) benzenesulfonamide; 4- methylbenzenesulfonic acid | 3-(trifluoro- methoxy) benzene- sulfonylchloride | 297.0 [M − TsOH + H]⁺ |
| B.146 | 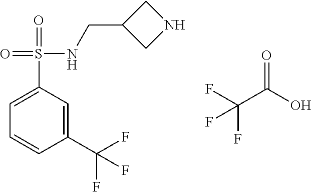 [SMILES: O=S(=O)(NCC1CNC1)c1cccc(C(F)(F)F)c1 ; O=C(O)C(F)(F)F] | N-(azetidin-3- ylmethyl)-3- (trifluoromethyl) benzenesulfonamide; trifluoroacetic acid | 3-(trifluoromethyl) benzenesulfonyl chloride | 295.2 [M − TFA + H]⁺ |
| B.147 | 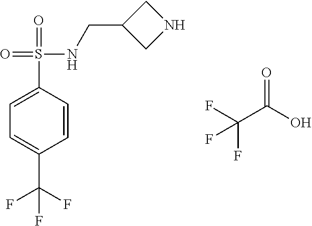 [SMILES: O=S(=O)(NCC1CNC1)c1ccc(C(F)(F)F)cc1 ; O=C(O)C(F)(F)F] | N-(azetidin-3- ylmethyl)-4- (trifluoromethyl) benzenesulfonamide; trifluoroacetic acid | 4-(trifluoromethyl) benzenesulfonyl chloride | 295.2 [M − TFA + H]⁺ |
| B.158 | 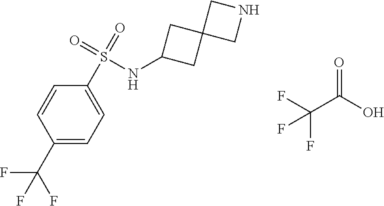 [SMILES: O=S(=O)(NC1CC2(CNC2)C1)c1ccc(C(F)(F)F)cc1 ; O=C(O)C(F)(F)F] | N-(2- azaspiro[3.3]heptan-6- yl)-4- (trifluoromethyl) benzenesulfonamide; trifluoroacetic acid | 4- (trifluoromethyl) benzenesulfonyl chloride | 321.1 [M − TFA + H]⁺ |
| B.159 | 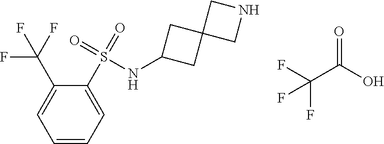 [SMILES: O=S(=O)(NC1CC2(CNC2)C1)c1ccccc1C(F)(F)F ; O=C(O)C(F)(F)F] | N-(2- azaspiro[3.3]heptan-6- yl)-2- (trifluoromethyl) benzenesulfonamide; trifluoroacetic acid | 2- (trifluoromethyl) benzenesulfonyl chloride | 321.1 [M − TFA + H]⁺ |
| B.160 | 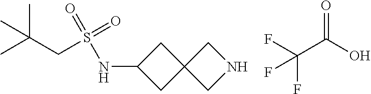 | N-(2- azaspiro[3.3]heptan-6- yl)-2,2-dimethyl- propane-1-sulfonamide; trifluoroacetic acid | 2,2- dimethyl propane- 1- sulfonyl chloride | 247.2 [M − TFA + H]⁺ |
| B.175 | 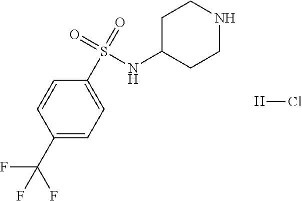 [SMILES: O=S(=O)(NC1CCNCC1)c1ccc(C(F)(F)F)cc1 ; Cl] | N-(4-piperidyl)-4- (trifluoromethyl) benzenesulfonamide; hydrochloride | 4- (trifluoromethyl) benzenesulfonyl chloride | 309.1 [M − HCl + H]⁺ |
| B.176 | 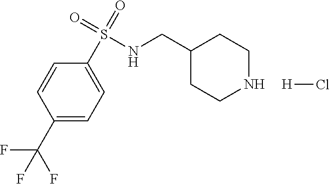 | N-(4-piperidylmethyl)- 4- (trifluoromethyl) benzenesulfonamide; hydrochloride | 4- (trifluoromethyl) benzenesuIfonyl chloride | 323.1 [M − HCl + H]⁺ |
| B.177 | 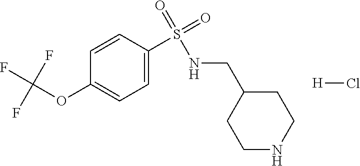 | N-(4-piperidylmethyl)- 4-(trifluoromethoxy) benzenesulfonamide; hydrochloride | 4- (trifluoromethoxy) benzene sulfonyl chloride | 339.1 [M − HCl + H]⁺ |
| B.178 | 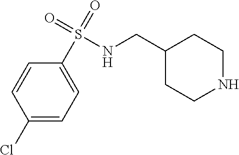 [SMILES: O=S(=O)(NCC1CCNCC1)c1ccc(Cl)cc1] | 4-chloro-N-(4- piperidylmethyl) benzenesulfonamide | 4-chlorobenzene- sulfonyl chloride | 288.8 [M + H]⁺ |

### Example B.31

### N-[6-(trifluoromethyl)pyridazin-3-yl]-2-azaspiro[3.3]heptan-6-amine; trifluoroacetic Acid

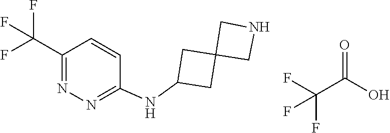

To a solution of 6-[[6-(trifluoromethyl)pyridazin-3-yl]amino]-2-azaspiro[3.3]heptane-2-carboxylic acid tert-butyl ester (708 mg, 1.94 mmol) in dichloromethane (8 mL) was added TFA (2.21 g, 1.49 mL, 19.4 mmol) and the reaction mixture was stirred at r.t for 18 h. Volatiles were removed in vacuo to yield 1310 mg of the crude title compound, purity roughly 55% with major contaminant excess TFA, which was used without further purification. MS (ESI): m/z=259.1 [M-TFA+H]⁺

### Step a) 6-[[6-(trifluoromethyl)pyridazin-3 yl]amino]-2-azaspiro[3.3]heptane-2-carboxylic Acid Tert-Butyl Ester

To a solution of 6-amino-2-azaspiro[3.3]heptane-2-carboxylic acid tert-butyl ester (832 mg, 3.92 mmol) in N,N-dimethylformamide (12 mL) was added DIPEA (690 mg, 932 μL, 5.34 mmol) and 3-chloro-6-(trifluoromethyl)pyridazine (650 mg, 3.56 mmol) after which the reaction mixture was stirred at 80° C. for 18 h. Volatiles were removed in vacuo and the crude residue was partitioned between ethyl acetate and sat. aq. NH₄Cl solution. The organic phase was collected and the aqueous phase was back-extracted with ethyl acetate. The combined organic phases were dried over sodium sulfate and evaporated down to dryness. The crude material was purified by flash chromatography, using an eluent mixture of dichloromethane and methanol (0% to 10%) to yield 708 mg of the title compound. MS (ESI): m/z=359.2 [M+H]⁺

### Example B.32

### 6-[[4-(trifluoromethyl)pyrazol-1-yl]methyl]-2-azaspiro[3.3]heptane; 4-methylbenzenesulfonic Acid

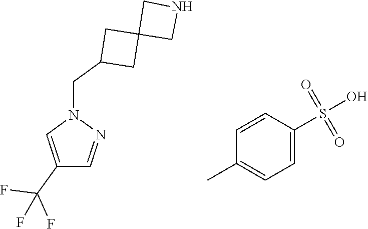 [SMILES: FC(F)(F)c1cnn(CC2CC3(CNC3)C2)c1 ; Cc1ccc(S(=O)(=O)O)cc1]

A solution of tert-butyl 6-[[4-(trifluoromethyl)pyrazol-1-yl]methyl]-2-azaspiro[3.3]heptane-2-carboxylate (675 mg, 1.95 mmol) and p-toluenesulfonic acid (404 mg, 2.35 mmol) in EtOAc (6 mL) was stirred at 80° C. for 12 h. The mixture was concentrated under vacuum to give a residue. To the residue was added deionized water and the mixture was lyophilized to give the title compound (794 mg, 96% yield) as a white solid. MS (ESI): m/z=246.2 [M-TsOH+H]⁺

### Step a) Tert-Butyl 6-[[4-(trifluoromethyl)pyrazol-1-yl]methylene]-2-azaspiro[3.3]heptane-2-carboxylate

To a solution of 4-(trifluoromethyl)-1H-pyrazole (2435 mg, 17.9 mmol), tert-butyl 6-[(4,4,5,5-tetramethyl-1,3,2-dioxaborolan-2-yl)methylene]-2-azaspiro[3.3]heptane-2-carboxylate (Example B.1, Step a)) (2000 mg, 5.97 mmol) and pyridine (1.45 mL, 17.9 mmol) in DMSO (80 mL) was added copper diacetate (2380 mg, 11.9 mmol) under 02 atmosphere, then stirred at 100° C. for 12 h under 02 (balloon) condition. The aqueous phase was extracted with ethyl acetate (200 mL×3). The combined organic phase was washed with brine (200 mL×3), dried with anhydrous Na₂SO₄, filtered and concentrated in vacuum. The residue was purified by flash silica gel chromatography (eluent of 0 to 30% ethyl acetate/petroleum ether) to give a crude product which was purified by reversed-phase HPLC (0.1% FA condition) to give the title compound (640 mg, 31% yield) as a brown solid. MS (ESI): m/z=288.1 [M-tBu+H]⁺

### Step b) Tert-Butyl 6-[[4-(trifluoromethyl)pyrazol-1-yl]methyl]-2-azaspiro[3.3]heptane-2-carboxylate

To a solution of tert-butyl 6-[[4-(trifluoromethyl)pyrazol-1-yl]methylene]-2-azaspiro[3.3]heptane-2-carboxylate (690 mg, 2.01 mmol) in EtOAc (7 mL) was added wet Pd/C (230 mg, 0.200 mmol), the mixture was stirred at 25° C. under H₂ atmosphere (balloon) for 2 h. The mixture was then filtered and the filtrate was concentrated to give the title compound (690 mg, 99% yield) as yellow solid. MS (ESI): m/z=346.1 [M+H]⁺

In analogy to Example B.32, the following building blocks were generated using the relevant (hetero)aryl building block for the Chan Lam-type coupling in Step 1. In some cases, alternative salts (e.g. trifluoroacetate, ditosylate, hydrochloride) were also used.

| | | | | MS, |
|---|---|---|---|---|
| | | | Building | ESI: |
| Ex. | Structure | Systematic Name | Blocks | m/z |
| B.36 | 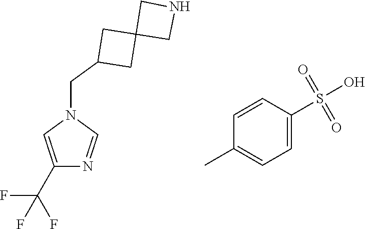 [SMILES: FC(F)(F)c1cn(CC2CC3(CNC3)C2)cn1 ; Cc1ccc(S(=O)(=O)O)cc1] | 6-[[4- (trifluoromethyl) imidazol-1-yl]methyl]-2- azaspiro[3.3]heptane; 4- methylbenzenesulfonic acid | 4- (trifluoro methyl)- 1H- imidazole (CAS: 33468- 69-8) | 246.3 [M − TsOH + H]⁺ |
| B.41 | 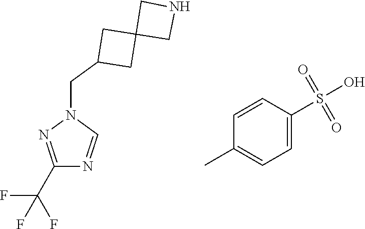 [SMILES: FC(F)(F)c1ncn(CC2CC3(CNC3)C2)n1 ; Cc1ccc(S(=O)(=O)O)cc1] | 6-[[3-(trifluoromethyl)- 1,2,4-triazol-1- yl]methyl]-2- azaspiro[3.3]heptane; 4- methylbenzenesulfonic acid | 3-(trifluoro methyl)- 1H- 1,2,4- triazole (CAS: 60406- 75-9) | 247.1 [M − TsOH + H]⁺ |
| B.417 | 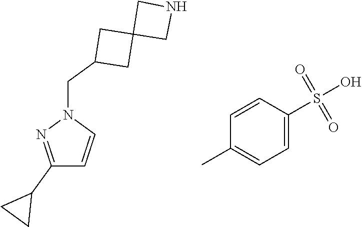 [SMILES: c1cn(CC2CC3(CNC3)C2)nc1C1CC1 ; Cc1ccc(S(=O)(=O)O)cc1] | 6-[(3- cyclopropylpyrazol-1- yl)methyl]-2- azaspiro[3.3] heptane; di 4- methylbenzenesulfonic acid | 5- cyclopropyl- 1H- pyrazole | 218.2 [M − 2TsOH + H]⁺ |

### Example B.37

### 7-[[1-(trifluoromethyl)cyclopropyl]methoxy]-2-azaspiro[3.5]nonane; 4-methylbenzenesulfonic Acid

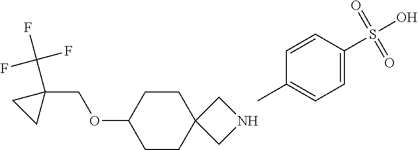

To a solution of 7-[[1-(trifluoromethyl)cyclopropyl]methoxy]-2-azaspiro[3.5]nonane-2-carboxylic acid tert-butyl ester (675 mg, 1.86 mmol) in isopropyl acetate (14 mL) was added p-toluenesulfonic acid monohydrate (424 mg, 2.23 mmol). The mixture was stirred at 80° C. for 5 h. The reaction mixture was concentrated in vacuo. Et₂O was added, and the mixture filtered through sintered glass. The white solid was washed twice with Et₂O and dried in vacuo to afford the title compound (752 mg, 88%) as a white solid MS (ESI): m/z=264.4 [M+H]⁺

### Step a) 7-[[1-(trifluoromethyl)cyclopropyl]methoxy]-2-azaspiro[3.5]nonane-2-carboxylic Acid Tert-Butyl Ester

To a solution of 7-hydroxy-2-azaspiro[3.5]nonane-2-carboxylic acid tert-butyl ester (CAS: 1363383-18-9) (0.600 g, 2.49 mmol) in N,N-dimethylformamide, extra dry (6 mL) was added sodium hydride (114 mg, 2.86 mmol). The mixture was stirred at room temp. for 1 h. 1-(bromomethyl)-1-(trifluoromethyl)cyclopropane (505 mg, 2.49 mmol) was added. The mixture was stirred at 80° C. for 16 h.

The reaction mixture was poured into EtOAc and washed with water and brine. The organic layer was dried over Na₂SO₄ and concentrated in vacuo. The crude material was purified by flash chromatography (eluting with 0% to 40% AcOEt in heptane) to afford the title compound (681 mg, 68%). MS (ESI): m/z=308.1 [M-tBu+H]⁺

In analogy to Example B.37, the following building blocks were generated using the relevant commercially available building blocks in step a).

| | | | | MS, |
|---|---|---|---|---|
| | | | | ESI: |
| Ex. | Structure | Systematic Name | Building Blocks | m/z |
| B.246 | 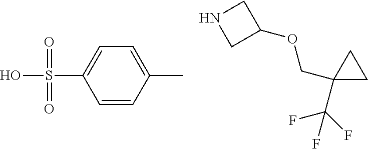 | 4- methylbenzenesulfonic acid;3-[1- (trifluoromethyl) cyclopropyl]methoxy] azetidine | tert-butyl 3- hydroxyazetidine-1- carboxylate (CAS RN: 141699-55-0) and 1- (bromomethyl)-1- (trifluoromethyl) cyclopropane (CAS RN: 1155272-93-7) | 196.0 [M + H]⁺ |

### Example B.39

### 6-[[1-(trifluoromethyl)cyclopropyl]methoxy]-2-azaspiro[3.3]heptane; 4-methylbenzenesulfonic Acid

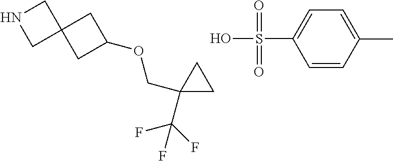

A solution of 6-[[1-(trifluoromethyl)cyclopropyl]methoxy]-2-azaspiro[3.3]heptane-2-carboxylic acid tert-butyl ester (633 mg, 1.89 mmol) in ethyl acetate (20 mL) was treated with p-toluenesulfonic acid monohydrate (366 mg, 1.93 mmol), at 23° C. The mixture was then heated to 80° C. for 18 h, before being cooled down to 23° C. and evaporated, to give 6-[[1-(trifluoromethyl)cyclopropyl]methoxy]-2-azaspiro[3.3]heptane 4-methylbenzenesulfonate (769 mg, 95.0%) as light yellow solid. MS (ESI): m/z=236.2 [M-C₇H₈O₃S+H]⁺

### Step a) 6-[[1-(trifluoromethyl)cyclopropyl]methoxy]-2-azaspiro[3.3]heptane-2-carboxylic Acid Tert-Butyl Ester

A solution of 6-hydroxy-2-azaspiro[3.3]heptane-2-carboxylic acid tert-butyl ester (500 mg, 2.34 mmol) in N,N-dimethylformamide, extra dry (10 mL) was treated with 1-(bromomethyl)-1-(trifluoromethyl)cyclopropane (476 mg, 2.34 mmol), at 23° C. under Ar. The mixture was stirred for another 30 min at this temperature, before being heated to 80° C. and stirred for 21.5 h. The mixture was then cooled down to 23° C., diluted with EtOAc, and the organic layer was washed with 1 M NaHCO₃ solution (1×), water (2×), and brine (1×). The organic layer was then dried over Na₂SO₄, filtered, and evaporated, to give the title compound (633 mg, 73%) as a crude colorless oil which was used directly without further purification. MS (ESI): m/z=280.2 [M+H-tBu]⁺

### Example B.40

### N-[1-(trifluoromethyl)cyclopropyl]-2-azaspiro[3.3]heptan-6-amine; 4 methylbenzenesulfonic Acid

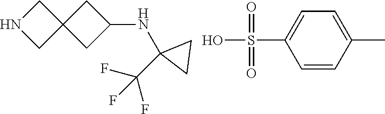

A solution of tert-butyl 6-[[1-(trifluoromethyl)cyclopropyl]amino]-2-azaspiro[3.3]heptane-2-carboxylate (880 mg, 2.75 mmol) and p-toluenesulfonic acid monohydrate (1568 mg, 8.24 mmol) in EtOAc (30 mL) was heated at reflux for 3 h, then cooled to room temp. and stirred for another 16 h. The obtained precipitate was collected by filtration, washed with EtOAc (15 mL) and dried under vacuum to provide the title compound (1335 mg, 84% yield) as a white solid. MS (ESI): m/z=221.2 [M-TsOH+H]⁺

### Step a) Tert-Butyl 6-[[1-(trifluoromethyl)cyclopropyl]amino]-2-azaspiro[3.3]heptane-2-carboxylate

To a stirred mixture of 1-(trifluoromethyl)cyclopropanamine hydrochloride (918 mg, 5.68 mmol) and tert-butyl 6-oxo-2-azaspiro[3.3]heptane-2-carboxylate (CAS: 1181816-12-5) (1.2 g, 5.68 mmol) in DCM (70 mL), triethylamine (2.38 mL, 17.0 mmol) was added. The reaction mixture was stirred at room temp. for 20 min. Then sodium triacetoxyborohydride (2410 mg, 11.4 mmol) was added to the solution in one portion and obtained mixture was stored for 18 h at 23° C. Then reaction mixture was diluted with DCM (50 mL), and 5% NaHCO₃(aq. sol.) (80 mL) was added. The organic phase was separated, and the aqueous layer was extracted with DCM (50 mL). The organic layers were combined, washed with brine (50 mL), dried over Na₂SO₄ and evaporated. Purification by FC (SiO₂; PE/MTBE) gave the title compound (70 mg, 3.7% yield) as a white solid. MS (ESI): m/z=321.2 [M+H]⁺

### Example B.42

### N-[1-(trifluoromethyl)cyclopropyl]-2-azaspiro[3.3]heptane-6-carboxamide; Hydrochloride

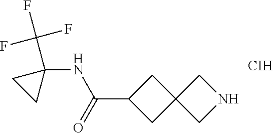

To a solution of benzyl 6-[[1-(trifluoromethyl)cyclopropyl]carbamoyl]-2-azaspiro[3.3]heptane-2-carboxylate (200 mg, 0.520 mmol) in methanol (10 ml-) was added palladium (10% on carbon) (0.02 mL, 0.190 mmol). The reaction mixture was stirred for 24 h at room temperature under a hydrogen atmosphere. The solids were removed by filtration and the filtrate was concentrated in vacuo. Then the residue was dissolved in THE (10 mL) and treated with 4 N HCl in dioxane, and stirred for 10 min at 23° C. The precipitate was collected by filtration to afford the title compound (60 mg, 40% yield) as a white solid. MS (ESI): m/z=249.2 [M+H]⁺

### Step a) Benzyl 6-[[1-(trifluoromethyl)cyclopropyl]carbamoyl]-2-azaspiro[3.3]heptane-2-carboxylate

To a stirred solution of 2-benzyloxycarbonyl-2-azaspiro[3.3]heptane-6-carboxylic acid (CAS: 1291487-33-6) (1.3 g, 4.72 mmol) and 1-(trifluoromethyl)cyclopropanamine hydrochloride (0.76 g, 4.72 mmol) in DMF (10 mL) were added HATU (2.33 g, 6.14 mmol) and TEA (2.3 mL, 16.5 mmol). The mixture was stinted overnight at 23° C. and then poured onto water and extracted with EtOAc (2×100 mL). The combined organic fractions were washed three times with water, dried under anhydrous sodium sulfate, and the solvent was removed under vacuum. The residue was triturated with 50 mL of MTBE to give the title compound (1 g, 55% yield) as a white solid. MS (ESI): m/z=383.2 [M+H]⁺

### Example B.51

### 6-[3-(trifluoromethyl)azetidin-1-yl]-2-azaspiro[3.3]heptane; 4-methylbenzenesulfonic

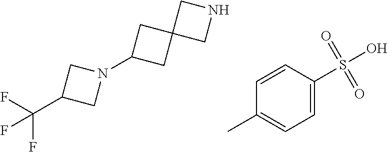

A solution of tert-butyl 6-[3-(trifluoromethyl)azetidin-1-yl]-2-azaspiro[3.3]heptane-2-carboxylate (550 mg, 1.72 mmol) and p-toluenesulfonic acid monobydrate (980 mg, 5.15 mmol) in EtOAc (50 mL) was heated at reflux for 2 h, then cooled to room temp. and stirred for another 16 h. The precipitate was collected by filtration, washed with ethyl acetate (20 mL) and dried under vacuum to provide the title compound (486 mg, 49% yield) as a white solid. MS (ESI): m/z=221.2 [M-TsOH+H]t

### Step a) Tert-Butyl 6-[3-(trifluoromethyl)azetidin-1 yl]-2-azaspiro[3.3]heptane-2-carboxylate

Tert-butyl 6-oxo-2-azaspiro[3.3]heptane-2-carboxylate (CAS: 1181816-12-5) (800 mg, 3.79 mmol), 3-(trifluoromethyl)azetidine; hydrochloride (CAS: 1221272-90-7) (612 mg, 3.79 mmol), and triethylamine (1.58 mL, 11.4 mmol) were mixed in DCM (40 mL) and stirred for 10 min at room temperature. Then sodium triacetoxyborohydride (1.61 g, 7.57 mmol) was added in one portion, and the reaction mixture was stirred for 18 h at room temperature. Then reaction mixture was diluted with DCM (50 mL), and 5% NaHCO₃ aq. sol. (80 mL) was added. The organic phase was separated, and the aqueous layer was extracted with DCM (50 mL). The organic layers were combined, washed with brine (50 mL), dried over Na₂SO₄ and evaporated. The cude product was purified by HPLC to obtain the title compound (550 mg, 44% yield) as a light yellow solid. MS (ESI): m/z=321.0 [M+H]⁺

In analogy to Example B.51, the following building blocks were generated using the relevant amine building block in Step a).

| | | | Building | MS, |
|---|---|---|---|---|
| | | | Blocks | ESI: |
| Ex. | Structure | Systematic Name | | m/z |
| B.52 | 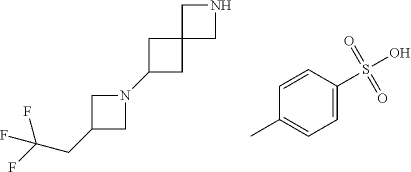 [SMILES: FC(F)(F)CC1CN(C2CC3(CNC3)C2)C1 ; Cc1ccc(S(=O)(=O)O)cc1] | 6-[3-(2,2,2- trifluoroethyl)azetidin- 1-yl]-2- azaspiro[3.3]heptane; 4- methylbenzenesulfonic acid | 3-(2,2,2- trifluoroethyl) azetidine; hydrochłoride (CAS: 1394041- 81-6) | 235.2 [M − TsOH + H]⁺ |
| B.155 | 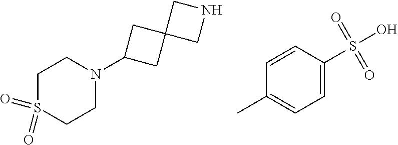 | 4-(2- azaspiro[3.3]heptan-6- yl)-1,4-thiazinane 1,1- dioxide; 4- methylbenzenesulfonic acid | thiomorp holine 1,1- dioxide hydrochloride | 231.2 [M − TsOH + H]⁺. |
| B.156 | 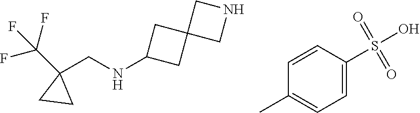 [SMILES: FC(F)(F)C1(CNC2CC3(CNC3)C2)CC1 ; Cc1ccc(S(=O)(=O)O)cc1] | N-[[1- (trifluoromethyl) cyclopropyl]methyl]-2- azaspiro[3.3]heptan-6- amine; 4- methylbenzenesulfonic acid | [1- (trifluoromethyl) cyclopropyl] methanamine; hydrochloride | 235.2 [M − TsOH + H]⁺ |

### Example B.53

### 7-[[4-(trifluoromethylsulfonyl)phenyl]methyl]-2,7-diazaspiro[3.5]nonane; trifluoroacetic Acid

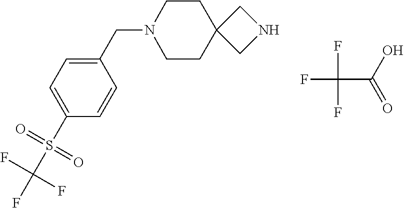 [SMILES: O=S(=O)(c1ccc(CN2CCC3(CC2)CNC3)cc1)C(F)(F)F ; O=C(O)C(F)(F)F]

To a solution of 7-(4-triflylbenzyl)-2,7-diazaspiro[3.5]nonane-2-carboxylic acid tert-butyl ester (360 mg, 0.803 mmol) in isopropyl acetate (6 mL) was added p-toluenesulfonic acid monohydrate (336 mg, 1.77 mmol). The mixture was stirred at 80° C. for 5 h. Et₂O was added, and the mixture filtered through sintered glass. The white solid was washed twice with Et₂O and dried in vacuo to afford the title compound (537 mg, 92%) as white solid. MS (ESI): m/z=349.1 [M−TFA+H]⁺

### Step a) 7-(4-triflylbenzyl)-2,7-diazaspiro[3.5]nonane-2-carboxylic Acid Tert-Butyl Ester

To a solution of 4-triflylbenzaldehyde (CAS: 650-89-5) (316 mg, 1.33 mmol) and 2,7-diazaspiro[3.5]nonane-2-carboxylic acid tert-butyl ester (CAS: 236406-55-6) (0.300 g, 1.33 mmol) in 1,2-dichloroethane (2.5 mL) was added sodium triacetoxy borohydride (309 mg, 1.46 mmol) and acetic acid (159 mg, 152 μL, 2.65 mmol). The mixture was stirred at room temp for 2 h. The reaction mixture was poured into EtOAc:THF 2:1 and washed with NaHCO₃sat. aq. sol., water and brine. The organic layer was dried over Na₂SO₄ and evaporated. Purification by FC (SiO₂; DCM/MeOH) gave the title compound (364 mg, 58%) as a white solid. MS (ESI): m/z=449.5[M+H]⁺

In analogy to Example B.53, the following building blocks were generated using the relevant building blocks in Step a).

| | | | | MS, |
|---|---|---|---|---|
| | | | Building | ESI: |
| Ex. | Structure | Systematic Name | Blocks | m/z |
| B.54 | 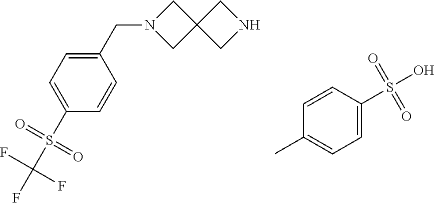 [SMILES: O=S(=O)(c1ccc(CN2CC3(CNC3)C2)cc1)C(F)(F)F ; Cc1ccc(S(=O)(=O)O)cc1] | 2-[[4- (trifluoromethyl- sulfonyl) phenyl]methyl]- 2,6- diazaspiro[3.3] heptane; 4- methylbenzene- sulfonic acid | 4- triflyl- benzaldehyde, tert-Butyl 2,6- diazaspiro [3.3]heptane- 2- carboxylate oxalate(2:1) (CAS 10410 26-71-4) | 321.1 [M − TsOH + H]⁺ |
| B.99 | 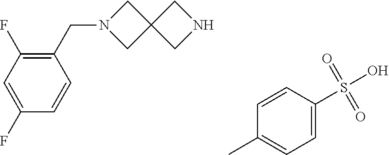 [SMILES: Fc1ccc(CN2CC3(CNC3)C2)c(F)c1 ; Cc1ccc(S(=O)(=O)O)cc1] | 2-[(2,4- difluorophenyl) methyl]-2,6- diazaspiro[3.3] heptane; 4- methylbenzene- sulfonic acid | tert-Butyl 2,6- diazaspiro [3.3]heptane- 2- carboxylate oxalate(2:1); 2,4- difluoro- benzaldehyde | 225.1 [M − TsOH + H]⁺ |
| B.100 | 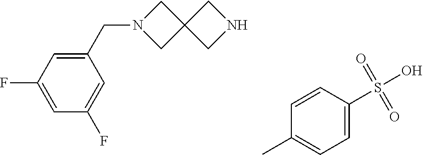 [SMILES: Fc1cc(F)cc(CN2CC3(CNC3)C2)c1 ; Cc1ccc(S(=O)(=O)O)cc1] | 2-[(3,5- difluorophenyl) methyl]-2,6- diazaspiro[3.3] heptane; 4- methylbenzene- sulfonic acid | tert-Butyl 2,6- diazaspiro [3.3]heptane- 2- carboxylate oxalate(2:1); 3,5- difluoro- benzaldehyde | 225.0 [M − TsOH + H]⁺ |
| B.102 | 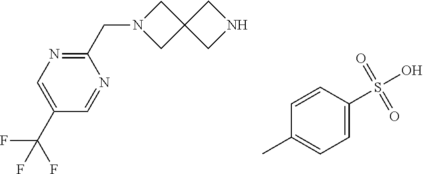 [SMILES: FC(F)(F)c1cnc(CN2CC3(CNC3)C2)nc1 ; Cc1ccc(S(=O)(=O)O)cc1] | 2-[[5- (trifluoromethyl) pyrimidin-2- yl]methyl]- 2,6- diazaspiro[3.3] heptane; 4- methylbenzene- sulfonic acid | tert-Butyl 2,6- diazaspiro [3.3]heptane- 2- carboxylate oxalate(2:1); 5- (trifluoro- meyhyl) pyrimidine- 2- carbaldehyde | 259.1 [M − TsOH + H]⁺ |
| B.104 | 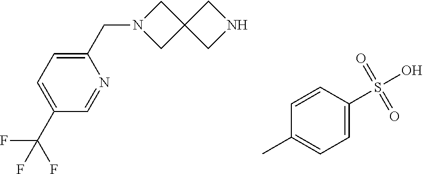 [SMILES: FC(F)(F)c1ccc(CN2CC3(CNC3)C2)nc1 ; Cc1ccc(S(=O)(=O)O)cc1] | 2-[[5- (trifluoromethyl)-2- pyridyl]methyl]-2,6- diazaspiro[3.3] heptane; 4- methylbenzene- sulfonic acid | tert-Butyl 2,6- diazaspiro [3.3]heptane- 2- carboxylate oxalate(2:1); 5- (trifluoro- methyl)-2- pyridine- carboxy- aldehyde | 258.4 [M − TsOH + H]⁺ |
| B.106 | 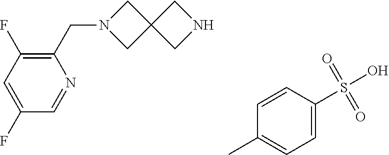 [SMILES: Fc1cnc(CN2CC3(CNC3)C2)c(F)c1 ; Cc1ccc(S(=O)(=O)O)cc1] | 2-[(3,5-difluoro-2- pyridyl)methyl]-2,6- diazaspiro[3.3] heptane; 4- methylbenzene- sulfonic acid | tert-Butyl 2,6- diazaspiro [3.3]heptane- 2- carboxylate oxalate(2:1); 3,5- difluoro- pyridine-2- carbaldehyde (CAS: 780801-58- 3) | 226.2 [M − TsOH + H]⁺ |
| B.107 | 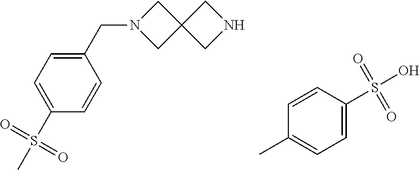 [SMILES: CS(=O)(=O)c1ccc(CN2CC3(CNC3)C2)cc1 ; Cc1ccc(S(=O)(=O)O)cc1] | 2-[(4- methylsulfonylphenyl) methyl]-2,6- diazaspiro[3.3] heptane; di 4- methylbenzene- sulfonic acid | tert-Butyl 2,6- diazaspiro [3.3]heptane- 2- carboxylate oxalate(2:1); 4- mesyl- benzaldehyde | 267.1 [M − 2TsOH + H]⁺ |
| B.108 | 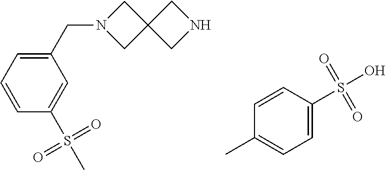 [SMILES: CS(=O)(=O)c1cccc(CN2CC3(CNC3)C2)c1 ; Cc1ccc(S(=O)(=O)O)cc1] | 2-[(3- methylsulfonylphenyl) methyl]-2,6- diazaspiro[3.3] heptane; di 4- methylbenzene- sulfonic acid | tert-Butyl 2,6- diazaspiro[3 .3]heptane- 2- carboxylate oxalate(2:1) ; 3- mesylbenzal dehyde | 267.1 [M − 2TsOH + H]⁺ |
| B.109 | 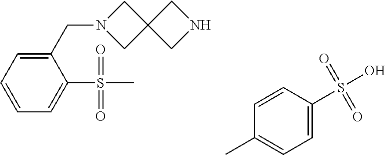 [SMILES: CS(=O)(=O)c1ccccc1CN1CC2(CNC2)C1 ; Cc1ccc(S(=O)(=O)O)cc1] | 2-[(2- methylsulfonylphenyl) methyl]-2,6- diazaspiro[3.3] heptane; di 4- methylbenzene- sulfonic acid | tert-Butyl 2,6- diazaspiro [3.3]heptane- 2- carboxylate oxalate(2:1); 2- mesylbenz- aldehyde | 267.1 [M − 2TsOH + H]⁺ |
| B.116 | 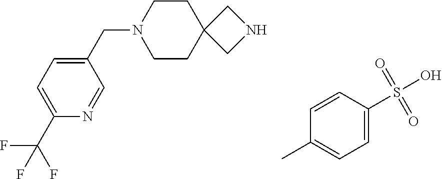 [SMILES: FC(F)(F)c1ccc(CN2CCC3(CC2)CNC3)cn1 ; Cc1ccc(S(=O)(=O)O)cc1] | 7-[[6- (trifluoromethyl)-3- pyridyl]methyl]-2,7- diazaspiro[3.5]nonane; 4- methylbenzene- sulfonic acid | 2,7- diazaspiro[3 5]nonane- 2- carboxylic acid tert- butyl ester; 6- (trifluorome thyl)nicotin aldehyde | 286.1 [M − TsOH + H]⁺ |
| B.117 | 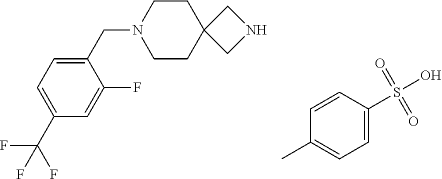 [SMILES: Fc1cc(C(F)(F)F)ccc1CN1CCC2(CC1)CNC2 ; Cc1ccc(S(=O)(=O)O)cc1] | 7-[[2-fluoro-4- (trifluoromethyl) phenyl]methyl]-2,7- diazaspiro[3.5] nonane; 4- methylbenzene- sulfonic acid | 2,7- diazaspiro [3.5]nonane- 2- carboxylic acid tert- butyl ester; 2-fluoro-4- (trifluoro- methyl) benzaldehyde | 303.1 [M − TsOH + H]⁺ |
| | 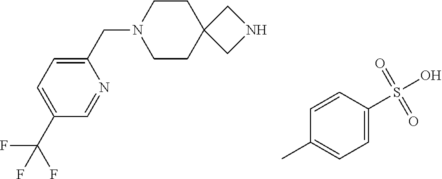 [SMILES: FC(F)(F)c1ccc(CN2CCC3(CC2)CNC3)nc1 ; Cc1ccc(S(=O)(=O)O)cc1] | 7-[[5- (trifluoromethyl)-2- pyridyl]methyl]-2,7- diazaspiro[3.5] nonane; 4- methylbenzene- sulfonic acid | 2,7- diazaspiro [3.5]nonane- 2- carboxylic acid tert- butyl ester; 5- (trifluoro- methyl) picolin aldehyde | 286.1 [M − TsOH + H]⁺ |
| B.119 | 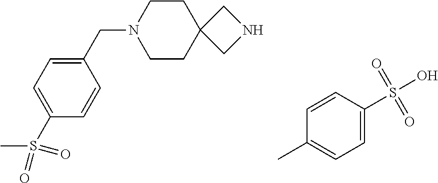 [SMILES: CS(=O)(=O)c1ccc(CN2CCC3(CC2)CNC3)cc1 ; Cc1ccc(S(=O)(=O)O)cc1] | 7-[(4- methylsulfonylphenyl) methyl]-2,7- diazaspiro[3.5] nonane; di 4- methylbenzene- sulfonic acid | 2,7- diazaspiro [3.5]nonane- 2- carboxylic acid tert- butyl ester; 4- mesylbenz- aldehyde | 295.2 [M − 2TsOH + H]⁺ |
| B.120 | 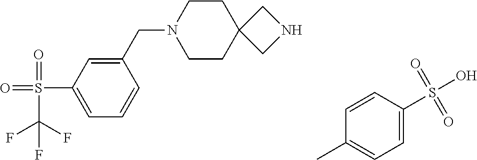 [SMILES: O=S(=O)(c1cccc(CN2CCC3(CC2)CNC3)c1)C(F)(F)F ; Cc1ccc(S(=O)(=O)O)cc1] | 7-[[3- (trifluoromethyl- sulfonyl)phenyl] methyl]- 2,7- diazaspiro[3.5] nonane; di 4- methylbenzene- sulfonic acid | 2,7- diazaspiro [3.5]nonane- 2- carboxylic acid tert- butyl ester; 3- triflylbenz- aldehyde (CAS: 1274904- 33-4) | 349.1 [M − 2TsOH + H]⁺ |
| B.121 | 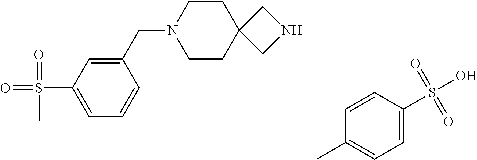 [SMILES: CS(=O)(=O)c1cccc(CN2CCC3(CC2)CNC3)c1 ; Cc1ccc(S(=O)(=O)O)cc1] | 7-[(3- methylsulfonylphenyl) methyl]-2,7- diazaspiro[3.5] nonane; 4- methylbenzene- sulfonic acid | 2,7- diazaspiro [3.5]nonane- 2- carboxylic acid tert- butyl ester; 3- mesylbenz- aldehyde | 295.2 [M − 2TsOH + H]⁺ |
| B.122 | 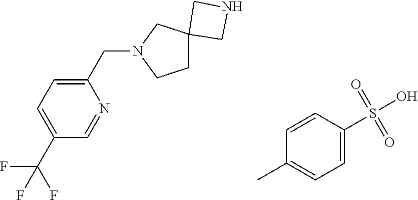 [SMILES: FC(F)(F)c1ccc(CN2CCC3(CNC3)C2)nc1 ; Cc1ccc(S(=O)(=O)O)cc1] | 6-[[5- (trifluoromethyl)-2- pyridyl]methyl]-2,6- diazaspiro[3.4] octane; 4- methylbenzene- sulfonic acid | tert-butyl 2,6- diazaspiro [3.4]octane-2- carboxylate; | 272.0 [M − 2TsOH + H]⁺ |
| B.123 | 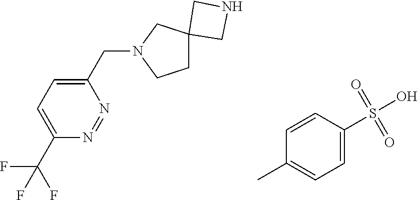 [SMILES: FC(F)(F)c1ccc(CN2CCC3(CNC3)C2)nn1 ; Cc1ccc(S(=O)(=O)O)cc1] | 6-[[6- (trifluoromethyl) pyridazin-3- yl]methyl]- 2,6- diazaspiro[3.4] octane; 4- methylbenzene- sulfonic acid | tert-butyl 2,6- diazaspiro [3.4]octane-2- carboxylate; 6- (trifluoro- methyl) pyridazine-3- carbaldehyde (CAS: 1245643- 49-5) | 273.0 [M − 2TsOH + H]⁺ |
| B.124 | 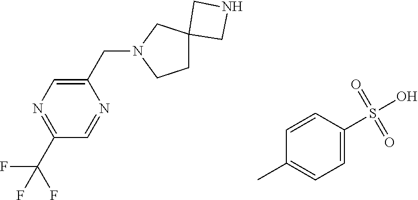 [SMILES: FC(F)(F)c1cnc(CN2CCC3(CNC3)C2)cn1 ; Cc1ccc(S(=O)(=O)O)cc1] | 6-[[5- (trifluoromethyl) pyrazin-2-yl] methyl]-2,6- diazaspiro[3.4] octane; 4- methylbenzene- sulfonic acid | tert-butyl 2,6- diazaspiro [3.4]octane-2- carboxylate; 5- (trifluoro- methyl) pyrazine-2- carbaldehyde (CAS: 1196151- 36-6) | 273.2 [M − 2TsOH + H]⁺ |
| B.125 | 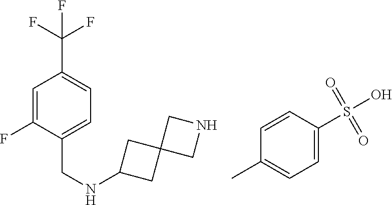 | N-[[2-fluoro-4- (trifluoromethyl) phenyl]methyl]-2- azaspiro[3.3]heptan- 6-amine; 4- methylbenzene- sulfonic acid | 6-amino-2- azaspiro[3.3] heptane-2- carboxylic acid tert- butyl ester; 2-fluoro-4- (trifluoro- methyl) benzaldehyde | 289.1 [M − TsOH + H]⁺ |
| B.126 | 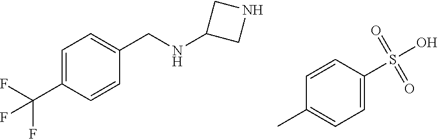 [SMILES: FC(F)(F)c1ccc(CNC2CNC2)cc1 ; Cc1ccc(S(=O)(=O)O)cc1] *Step a) used AcOH followed by sodium cyanoborohydride | N-[[4- (trifluoromethyl) phenyl]methyl] azetidin- 3-amine; 4- methylbenzene- sulfonic acid | 1-BOC-3- (amino) azetidine; 4- (trifluoro- methyl) benzaldehyde | 231.0 [M − TsOH + H]⁺ |
| B.127 | 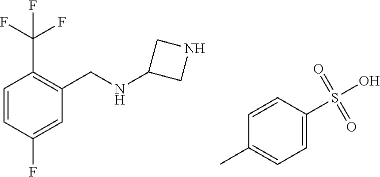 [SMILES: Fc1ccc(C(F)(F)F)c(CNC2CNC2)c1 ; Cc1ccc(S(=O)(=O)O)cc1] *Step a) used AcOH followed by sodium cyanoborohydride | N-[5-fluoro-2- (trifluoromethyl) phenyl]methyl] azetidin- 3-amine; 4- methylbenzene- sulfonic acid | 1-BOC-3- (amino) azetidine; 5- fluoro-2- (trifluoro- methyl) benzaldehyde | 249.2 [M − TsOH + H]⁺ |
| B.128 | 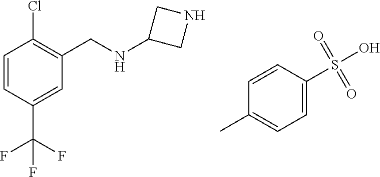 [SMILES: FC(F)(F)c1ccc(Cl)c(CNC2CNC2)c1 ; Cc1ccc(S(=O)(=O)O)cc1] *Step a) used AcOH followed by sodium cyanoborohydride | N-[[2-chloro-5- (trifluoromethyl) phenyl]methyl] azetidin- 3-amine; 4- methylbenzene- sulfonic acid | 1-BOC-3- (amino) azetidine; 2- chloro-5- (trifluoro- methyl) benzaldehyde | 265.1 [M − TsOH + H]⁺ |

| | | | | |
|---|---|---|---|---|
| B.129 | 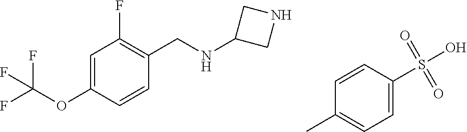 [SMILES: Fc1cc(OC(F)(F)F)ccc1CNC1CNC1 ; Cc1ccc(S(=O)(=O)O)cc1] *Step a) used AcOH followed by sodium cyanoborohydride | N-[[2-fluoro-4- (trifluoromethoxy) phenyl]methyl] azetidin- 3-amine; 4- methylbenzene- sulfonic acid | 1-BOC-3- (amino) azetidine; 2- fluoro-4- (trifluoro- methoxy) benzaldehyde | 265.1 [M − TsOH + H]⁺ |
| B.130 | 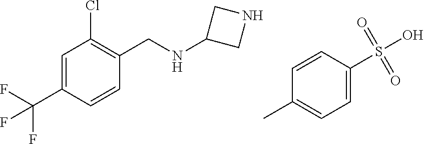 [SMILES: FC(F)(F)c1ccc(CNC2CNC2)c(Cl)c1 ; Cc1ccc(S(=O)(=O)O)cc1] *Step a) used AcOH followed by sodium cyanoborohydride | N-[[2-chloro-4- (trifluoromethyl) phenyl]methyl] azetidin- 3-amine; 4- methylbenzene- sulfonic acid | 1-BOC-3- (amino) azetidine; 2- chloro-4- (trifluoro- methyl) benzaldehyde | 265.1 [M − TsOH + H]⁺ |
| B.131 | 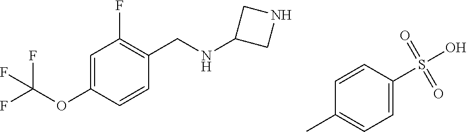 [SMILES: Fc1cc(OC(F)(F)F)ccc1CNC1CNC1 ; Cc1ccc(S(=O)(=O)O)cc1] *Step a) used AcOH followed by sodium cyanoborohydride | N-[[4- (trifluoromethoxy) phenyl]methyl] azetidin- 3-amine; 4- methylbenzene- sulfonic acid | 1-BOC-3- (amino) azetidine; 4- (trifluoro- methoxy) benzaldehyde | 247.1 [M − TsOH + H]⁺ |
| B.132 | 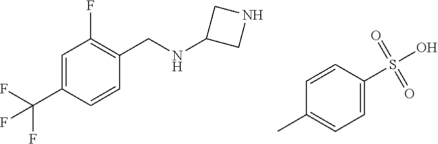 [SMILES: Fc1cc(C(F)(F)F)ccc1CNC1CNC1 ; Cc1ccc(S(=O)(=O)O)cc1] | N-[2-fluoro-4- (trifluoromethyl) phenyl]methyl] azetidin- 3-amine; 4- methylbenzene- sulfonic acid | 1-BOC-3- (amino) azetidine; 2- fluoro-4- (trifluoro- methyl) benzaldehyde | 249.1 [M − TsOH + H]⁺ |
| B.133 | 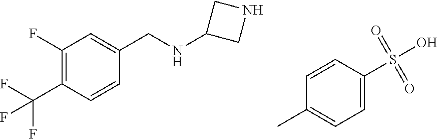 [SMILES: Fc1cc(CNC2CNC2)ccc1C(F)(F)F ; Cc1ccc(S(=O)(=O)O)cc1] *Step a) used AcOH followed by sodium cyanoborohydride | N-[[3-fluoro-4- (trifluoromethyl) phenyl]methyl] azetidin- 3-amine; 4- methylbenzene- sulfonic acid | 1-BOC-3- (amino) azetidine; 3- fluoro-4- (trifluoro- methyl) benzaldehyde | 249.1 [M − TsOH + H]⁺ |
| B.134 | 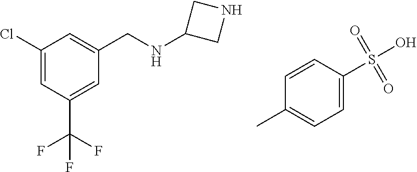 [SMILES: FC(F)(F)c1cc(Cl)cc(CNC2CNC2)c1 ; Cc1ccc(S(=O)(=O)O)cc1] *Step a) used AcOH followed by sodium cyanoborohydride | N-[[3-chloro-5- (trifluoromethyl) phenyl]methyl] azetidin- 3-amine; 4- methylbenzene- sulfonic acid | 1-BOC-3- (amino) azetidine; 3- chloro-5- (trifluoro- methyl) benzaldehyde | 265.0 [M − TsOH + H]⁺ |
| B.135 | 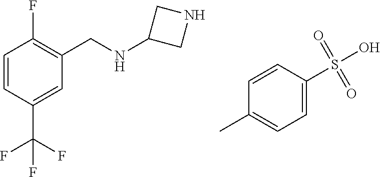 [SMILES: Fc1ccc(C(F)(F)F)cc1CNC1CNC1 ; Cc1ccc(S(=O)(=O)O)cc1] *Step a) used AcOH followed by sodium cyanoborohydride | N-[[2-fluoro-5- (trifluoromethyl) phenyl]methyl] azetidin- 3-amine; 4- methylbenzene- sulfonic acid | 1-BOC-3- (amino) azetidine; 2- fluoro-5- (trifluoro- methyl) benzaldehyde | 249.0 [M − TsOH + H]⁺ |
| B.136 | 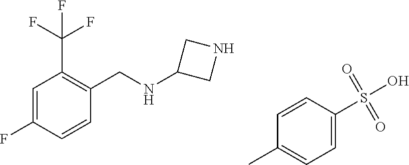 [SMILES: Fc1ccc(CNC2CNC2)c(C(F)(F)F)c1 ; Cc1ccc(S(=O)(=O)O)cc1] *Step a) used AcOH followed by sodium cyanoborohydride | N-[[4-fluoro-2- (trifluoromethyl) phenyl]methyl] azetidin- 3-amine; 4- methylbenzene- sulfonic acid | 1-BOC-3- (amino) azetidine; 4- fluoro-2- (trifluoro- methyl) benzaldehyde | 249.1 [M − TsOH + H]⁺ |
| B.137 | 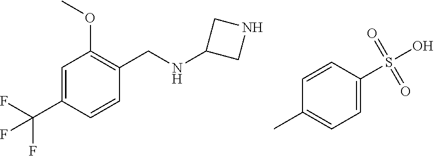 [SMILES: COc1cc(C(F)(F)F)ccc1CNC1CNC1 ; Cc1ccc(S(=O)(=O)O)cc1] *Step a) used AcOH followed by sodium cyanoborohydride | N-[2-methoxy-4- (trifluoromethyl) phenyl]methyl] azetidin- 3-amine; 4- methylbenzene- sulfonic acid | 1-BOC-3- (amino) azetidine; 2- methoxy-4- (trifluoro- methyl) benzaldehyde | 261.1 [M − TsOH + H]⁺ |
| B.138 | 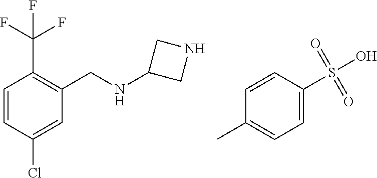 [SMILES: FC(F)(F)c1ccc(Cl)cc1CNC1CNC1 ; Cc1ccc(S(=O)(=O)O)cc1] *Step a) used AcOH followed by sodium cyanoborohydride | N-[[5-chloro-2- (trifluoromethyl) phenyl]methyl] azetidin- 3-amine; 4- methylbenzene- sulfonic acid | 1-BOC-3- (amino) azetidine; 5- chloro-2- (trifluoro- methyl) benzaldehyde | 265.1 [M − TsOH + H]⁺ |
| B.326 | 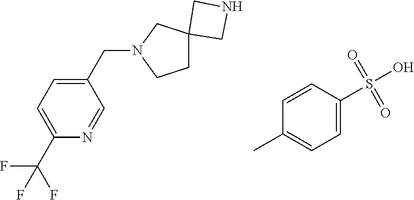 [SMILES: FC(F)(F)c1ccc(CN2CCC3(CNC3)C2)cn1 ; Cc1ccc(S(=O)(=O)O)cc1] | 6-[[6- (trifluoromethyl)-3- pyridyl]methyl]-2,6- diazaspiro[3.4] octane; di 4- methylbenzene- sulfonic acid | tert-butyl 2,6- diazaspiro [3.4]octane-2- carboxylate; 6- (trifluoro- methyl) pyridine-3- carboxaldehyde | 272.4 [M − 2TsOH + H]⁺ |
| B.347 | 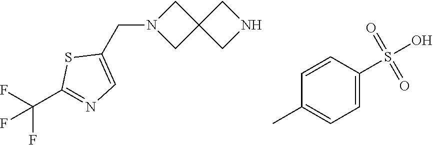 [SMILES: FC(F)(F)c1ncc(CN2CC3(CNC3)C2)s1 ; Cc1ccc(S(=O)(=O)O)cc1] | 5-(2,6- diazaspiro[3.3] heptan- 2-ylmethyl)-2- (trifluoromethyl) thiazole; di 4- methylbenzene- sulfonic acid | tert-Butyl 2,6- diazaspiro [3.3]heptane- 2- carboxylate oxalate(2:1) (CAS: 1374659- 01-4); 2- (trifluoro- methyl) thiazole-5- carbaldehyde (CAS: 903130-38- 1) | 264.0 [M − 2TsOH + H]⁺ |
| B.350 | 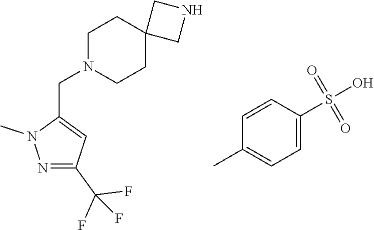 [SMILES: Cn1nc(C(F)(F)F)cc1CN1CCC2(CC1)CNC2 ; Cc1ccc(S(=O)(=O)O)cc1] | 7-[3- (trifluoromethyl)- 1H-pyrazol-5- yl]methyl]-2,7- diazaspiro[3.5] nonane; di 4- methylbenzene- sulfonic acid | 2-(tert- butoxycarbonyl)- 2,7- diazaspiro[3.5] nonane; 2-methyl-5- (trifluoro- methyl) pyrazole-3- carbaldehyde (CAS: 1414962- 92-7) | 289.1 [M − 2TsOH + H]⁺ |
| B.351 | 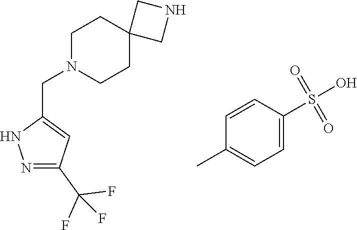 [SMILES: FC(F)(F)c1cc(CN2CCC3(CC2)CNC3)[nH]n1 ; Cc1ccc(S(=O)(=O)O)cc1] | 7-[[3- (trifluoromethyl)- 1H-pyrazol-5- yl]methyl]-2,7- diazaspiro[3.5] nonane; di 4- methylbenzene- sulfonic acid | 2-(tert- butoxycarbonyl)- 2,7- diazaspiro [3.5]nonane; 3- (trifluoro- methyl)-1H- pyrazole-5- carbaldehyde (CAS: 591234-14- 9) | 275.2 [M − 2TsOH + H]⁺ |
| B.352 | 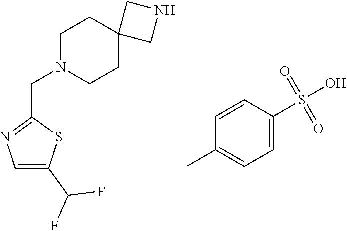 [SMILES: FC(F)c1cnc(CN2CCC3(CC2)CNC3)s1 ; Cc1ccc(S(=O)(=O)O)cc1] | 2-(2,7- diazaspiro[3.5]nonan- 7-ylmethyl)-5- (difluoromethyl) thiazole; 4- methylbenzene- sulfonic acid | 2-(tert- butoxycarbonyl)- 2,7- diazaspiro [3.5]nonane; 5- (difluoro- methyl) thiazole-2- carbaldehyde (CAS: 1780912- 17-5) | 274.0 [M − TsOH + H]⁺ |
| B.362 | 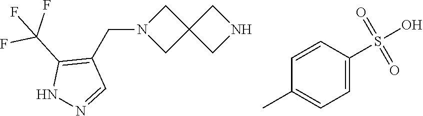 [SMILES: FC(F)(F)c1[nH]ncc1CN1CC2(CNC2)C1 ; Cc1ccc(S(=O)(=O)O)cc1] | 2-[[5- (trifluoromethyl)- 1H-pyrazol-4- yl]methyl]-2,6- diazaspiro[3.3] heptane; 4- methylbenzene- sulfonic acid | tert-Butyl 2,6- diazaspiro [3.3]heptane- 2- carboxylate oxalate(2:1) (CAS: 1374659- 01-4); 5- (trifluoro- methyl)-1H- pyrazole-4- carbaldehyde (CAS: 1001020- 14-9) | 247.0 [M − TsOH + H]⁺ |
| B.363 | 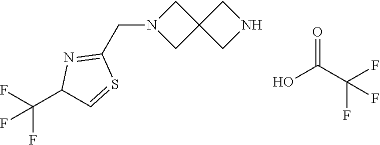 | 2-(2,6- diazaspiro[3.3]heptan- 2-ylmethyl)-4- (trifluoromethyl) thiazole; 2,2,2- trifluoroacetic acid | tert-butyl 2,6- diazaspiro [3.3]heptane- 2- carboxylate hydrochloride; 4- (trifluoro- methyl) thiazole-2- carbaldehyde (CAS: 354587-75- 0) | 264.0 [M − TFA + H]⁺ |
| B.364 | 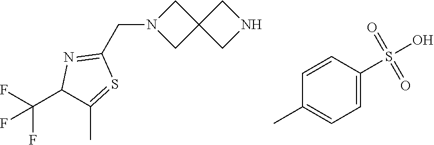 | 2-(2,6- diazaspiro[3.3]heptan- 2-ylmethyl)-5- methyl-4- (trifluoromethyl) thiazole; 4- methylbenzene- sulfonic acid | tert-butyl 2,6- diazaspiro [3.3]heptane- 2- carboxylate hydrochloride; 5- methyl-4- (trifluoro- methyl)-1,3- thiazole-2- carbaldehyde (CAS: 1379099- 99-6) | 278.0 [M − TsOH + H]⁺ |
| B.365 | 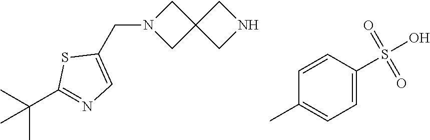 [SMILES: CC(C)(C)c1ncc(CN2CC3(CNC3)C2)s1 ; Cc1ccc(S(=O)(=O)O)cc1] | 2-tert-butyl-5-(2,6- diazaspiro[3.3] heptan- 2- ylmethyl)thiazole; 4- methylbenzene- sulfonic acid | tert-butyl 2,6- diazaspiro[3.3] heptane- 2- carboxylate; oxalic acid; 2-tert- butylthiazole- 5- carbaldehyde (CAS: 1211695- 52-1) | 252.0 [M − TsOH + H]⁺ |
| B.375 | 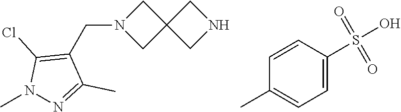 [SMILES: Cc1nn(C)c(Cl)c1CN1CC2(CNC2)C1 ; Cc1ccc(S(=O)(=O)O)cc1] | 2-[(5-chloro-1,3- dimethyl-pyrazol-4- yl)methyl]-2,6- diazaspiro[3.3] heptane; 4- methylbenzene- sulfonic acid | 2,6- diazaspiro [3.3]heptane- 2- carboxylic acid tert- butyl ester; 5-chloro- 1,3- dimethyl- pyrazole-4- carbaldehyde (CAS: 27006-76-4) | 241.1 [M − TsOH + H]⁺ |
| B.380 | 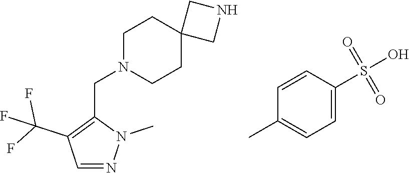 [SMILES: Cn1ncc(C(F)(F)F)c1CN1CCC2(CC1)CNC2 ; Cc1ccc(S(=O)(=O)O)cc1] | 7-[2-methyl-4- (trifluoromethyl) pyrazol-3-yl]methyl]- 2,7- diazaspiro[3.5] nonane; 4- methylbenzene- sulfonic acid | 2-(tert- butoxycarbonyl)- 2,7- diazaspiro [3.5]nonane; 1-methyl-4- (trifluoro- methyl)-1H- pyrazole-5- carbaldehyde (CAS: 1780459- 24-6) | 289.2 [M − TsOH + H]⁺ |
| B.385 | 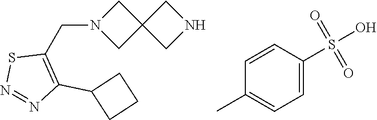 [SMILES: C1CC(c2nnsc2CN2CC3(CNC3)C2)C1 ; Cc1ccc(S(=O)(=O)O)cc1] | 4-cyclobutyl-5-(2,6- diazaspiro[3.3] heptan-2- ylmethyl)thiadiazole; 4- methylbenzene- sulfonic acid | tert-butyl 2,6- diazaspiro[3.3] heptane- 2- carboxylate hydrochloride; 4- cyclobutyl- thiadiazole-5- carbaldehyde (CAS: 2639443- 82-4) | 251.2 [M − TsOH + H]⁺ |
| B.391 | 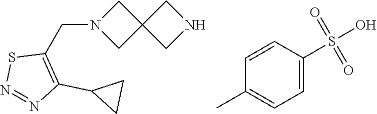 [SMILES: C1CC1c1nnsc1CN1CC2(CNC2)C1 ; Cc1ccc(S(=O)(=O)O)cc1] | 4-cyclopropyl-5- (2,6- diazaspiro[3.3] heptan-2- ylmethyl)thiadiazole; 4- methylbenzene- sulfonic acid | tert-butyl 2,6- diazaspiro [3.3]heptane- 2- carboxylate hydrochloride; 4- cyclopropyl thiadiazole- 5- carbaldehyde (CAS: 1000878- 41-0) | 237.0 [M − TsOH + H]⁺ |
| B.395 | 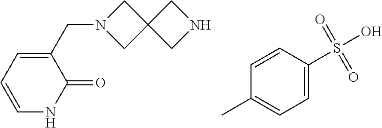 | 3-(2,6- diazaspiro[3.3]heptan- 2-ylmethyl)-1H- pyridin-2-one; 4- methylbenzene- sulfonic acid | tert-butyl 2,6- diazaspiro[3.3] heptane- 2- carboxylate hydrochloride; | 206.2 [M − TsOH + H]⁺ |
| B.397 | 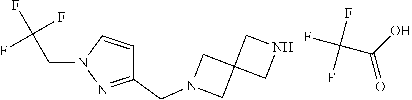 | 2-[1-(2,2,2- trifluoroethyl)pyrazol- 3-yl]methyl]-2,6- diazaspiro[3.3] heptane; 2,2,2- trifluoroacetic acid | tert-butyl 2,6- diazaspiro [3.3]heptane- 2- carboxylate hydrochloride; 1-(2,2,2- trifluoroethyl) pyrazole- 3- carbaldehyde (CAS: 1310379- 34-0) | 261.2 [M − TFA + H]⁺ |
| B.402 | 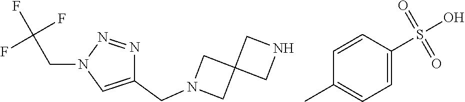 | 2-[[1-(2,2,2- trifluoroethyl)triazol- 4-yl]methyl]-2,6- diazaspiro[3.3] heptane; 4- methylbenzene- sulfonic acid | tert-butyl 2,6- diazaspiro [3.3]heptane- 2- carboxylate hydrochloride; 1-(2,2,2- trifluoro- ethyl)triazole- 4- carbaldehyde (CAS: 1393568- 30-3) | 262.0 [M − TsOH + H]⁺ |
| B.403 | 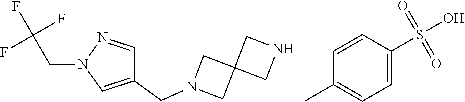 | 2-[ 1-(2,2,2- trifluoroethyl)pyrazol- 4-yl]methyl]-2,6- diazaspiro[3.3] heptane; 4- methylbenzene- sulfonic acid | tert-butyl 2,6- diazaspiro [3.3]heptane- 2- carboxylate hydrochloride; 1-(2,2,2- trifluoroethyl) pyrazole- 4- carbaldehyde (CAS: 790254-33- 0) | 261.0 [M − TsOH + H]⁺ |
| B.404 | 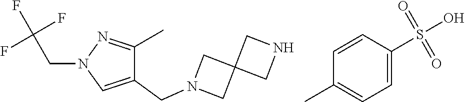 | 2-[[3-methyl-1- (2,2,2- trifluoroethyl)pyrazol- 4-yl]methyl]-2,6- diazaspiro[3.3] heptane; di 4- methylbenzene- sulfonic acid | tert-butyl 2,6- diazaspiro [3.3]heptane- 2- carboxylate; oxalic acid; 3-methyl-1- (2,2,2- trifluoroethyl) pyrazole- 4- carbaldehyde (CAS: 1006471- 12-0) | 275.1 [M − 2TsOH + H]⁺ |
| B.405 | 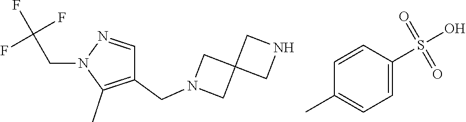 [SMILES: Cc1c(CN2CC3(CNC3)C2)cnn1CC(F)(F)F ; Cc1ccc(S(=O)(=O)O)cc1] | 2-[5-methyl-1- (2,2,2- trifluoroethyl)pyrazol- 4-yl]methyl]-2,6- diazaspiro[3.3] heptane; di 4- methylbenzene- sulfonic acid | tert-butyl 2,6- diazaspiro [3.3]heptane- 2- carboxylate; oxalic acid; 5-methyl-1- (2,2,2- trifluoroethyl) pyrazole- 4- carbaldehyde (CAS: 1936401- 01-2) | 275.2 [M − 2TsOH + H]⁺ |
| B.406 | 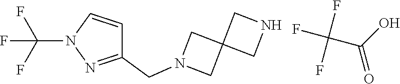 | 2-[[1- (trifluoromethyl) pyrazol-3-yl]methyl]- 2,6- diazaspiro[3.3] heptane; 2,2,2- trifluoroacetic acid | tert-butyl 2,6- diazaspiro [3.3]heptane- 2- carboxylate hydrochloride; 1- (trifluoro- methyl)pyrazole- 3- carbaldehyde (CAS: 2091951- 89-0) | 247.2 [M − TFA + H]⁺ |
| B.420 | 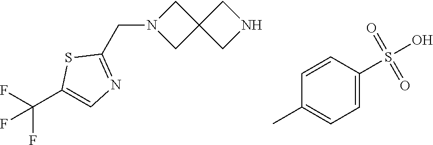 [SMILES: FC(F)(F)c1cnc(CN2CC3(CNC3)C2)s1 ; Cc1ccc(S(=O)(=O)O)cc1] | 2-(2,6- diazaspiro[3.3]heptan- 2-ylmethyl)-5- (trifluoromethyl) thiazole; 4- methylbenzene- sulfonic acid | tert-butyl 2,6- diazaspiro [3.3]heptane- 2- carboxylate hydrochloride; 5- (trifluoro- methyl)thiazole- 2- carbaldehyde (CAS: 1379174- 19-2) | 264.2 [M − TsOH + H]⁺ |
| B.421 | 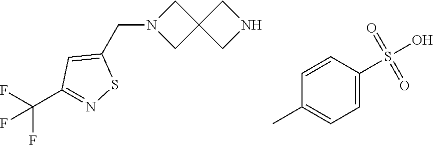 [SMILES: FC(F)(F)c1cc(CN2CC3(CNC3)C2)sn1 ; Cc1ccc(S(=O)(=O)O)cc1] | 5-(2,6- diazaspiro[3.3]heptan- 2-ylmethyl)-3- (trifluoromethyl) isothiazole; 4- methylbenzene- sulfonic acid | tert-butyl 2,6- diazaspiro [3.3]heptane- 2- carboxylate hydrochloride; 3- (trifluoro- methyl) isothiazole-5- carbaldehyde (CAS: 1784002- 13-6) | 264.2 [M − TsOH + H]⁺ |
| B.423 | 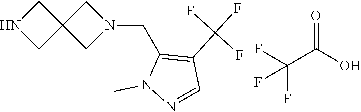 | 2-[2-methyl-4- (trifluoromethyl) pyrazol-3-yl]methyl]- 2,6- diazaspiro[3.3] heptane; 2,2,2- trifluoroacetic acid | tert-butyl 2,6- diazaspiro [3.3]heptane- 2- carboxylate hydrochloride; 2- methyl-4- (trifluoro- methyl) pyrazole-3- carbaldehyde (CAS: 1780459- 24-6) | 261.2 [M − TFA + H]⁺ |
| B.425 | 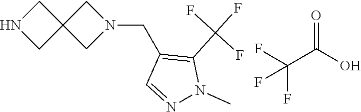 | 2-[1-methyl-5- (trifluoromethyl) pyrazol-4-yl]methyl]- 2,6- diazaspiro[3.3] heptane; 2,2,2- trifluoroacetic acid | tert-butyl 2,6- diazaspiro [3.3]heptane- 2- carboxylate hydrochloride; 1- methyl-5- trifluoro- methyl-4- pyrazole- carbaldehyde (CAS: 497833-04- 2) | 261.2 [M − TFA + H]⁺ |
| B.426 | 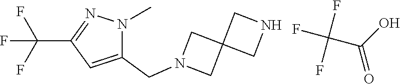 | 2-[[2-methyl-5- (trifluoromethyl) pyrazol-3-yl]methyl]- 2,6- diazaspiro[3.3] heptane; 2,2,2- trifluoroacetic acid | tert-butyl 2,6- diazaspiro [3.3]heptane- 2- carboxylate hydrochloride; 2- methyl-5- (trifluoro- methyl) pyrazole-3- carbaldehyde (CAS: 1414962- 92-7) | 261.2 [M − TFA + H]⁺ |
| B.427 | 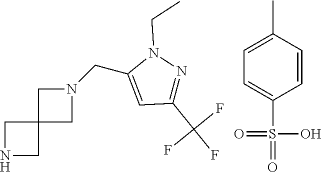 | 2-[2-ethyl-5- (trifluoromethyl) pyrazol-3-yl]methyl]- 2,6- diazaspiro[3.3] heptane; 4- methylbenzene- sulfonic acid | tert-butyl 2,6- diazaspiro [3.3]heptane- 2- carboxylate hydrochloride; 1-ethyl- 3- (trifluoro- methyl)-1H- pyrazole-5- carbaldehyde (CAS: 1443279- 59-1) | 275.2 [M − TsOH + H]⁺ |
| B.429 | 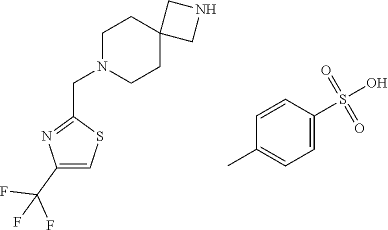 [SMILES: FC(F)(F)c1csc(CN2CCC3(CC2)CNC3)n1 ; Cc1ccc(S(=O)(=O)O)cc1] | 2-(2,7- diazaspiro[3.5]nonan- 7-ylmethyl)-4- (trifluoromethyl) thiazole; di 4- methylbenzene- sulfonic acid | 2-(tert- butoxycarbonyl)- 2,7- diazaspiro [3.5]nonane; 4- (trifluoro- methyl) thiazole-2- carbaldehyde (CAS: 354587-75- 0) | 292.0 [M − 2TsOH + H]⁺ |
| B.430 | 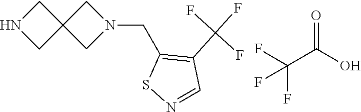 | 5-(2,6- diazaspiro[3.3]heptan- 2-ylmethyl)-4- (trifluoromethyl) isothiazole; 2,2,2- trifluoroacetic acid | tert-butyl 2,6- diazaspiro [3.3]heptane- 2- carboxylate hydrochloride; 4- (trifluoro- methyl) isothiazole-5- carbaldehyde (CAS: 1822992- 91-5) | 264.0 [M − TFA + H]⁺ |
| B.431 | 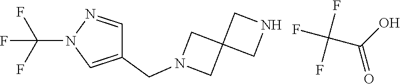 | 2-[[1- (trifluoromethyl) pyrazol-4-yl]methyl]- 2,6- diazaspiro[3.3] heptane; 2,2,2- trifluoroacetic acid | tert-butyl 2,6- diazaspiro [3.3]heptane- 2- carboxylate hydrochloride; 1- (trifluoro- methyl)pyrazol- 4- carbaldehyde (CAS: 1556454- 55-7) | 347.2 [M − TFA + H]⁺ |
| B.434 | 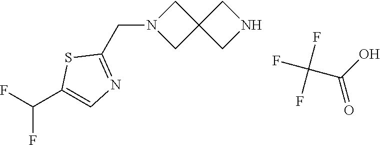 | 2-(2,6- diazaspiro[3.3]heptan- 2-ylmethyl)-5- (difluoromethyl) thiazole; 2,2,2- trifluoroacetic acid | tert-butyl 2,6- diazaspiro [3.3]heptane- 2- carboxylate hydrochloride; 5- (difluoro- methyl)-1,3- thiazole-2- carbaldehyde (CAS: 1780912- 17-5) | 246.2 [M − TFA + H]⁺ |
| B.436 | 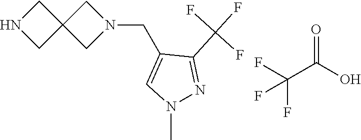 | 2-[[1-methyl-3- (trifluoromethyl) pyrazol-4-yl]methyl]- 2,6- diazaspiro[3.3] heptane; 2,2,2- trifluoroacetic acid | tert-butyl 2,6- diazaspiro [3.3]heptane- 2- carboxylate; oxalic acid; 1-methyl-3- (trifluoro- methyl) pyrazole-4- carbaldehyde (CAS: 128225-66- 1) | 261.1 [M − TFA + H]⁺ |
| B.438 | 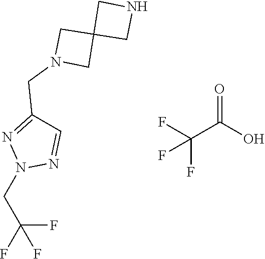 [SMILES: FC(F)(F)Cn1ncc(CN2CC3(CNC3)C2)n1 ; O=C(O)C(F)(F)F] | 2-[[2-(2,2,2- trifluoroethyl)triazol- 4-yl]methyl]-2,6- diazaspiro[3.3] heptane; 2,2,2- trifluoroacetic acid | tert-butyl 2,6- diazaspiro [3.3]heptane- 2- carboxylate hydrochloride; 2-(2,2,2- trifluoro- ethyl)triazole- 4- carbaldehyde (CAS: 128225-66- 1) | 262.2 [M − TFA + H]⁺ |
| B.440 | 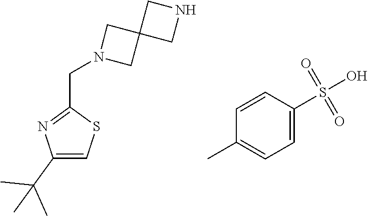 [SMILES: CC(C)(C)c1csc(CN2CC3(CNC3)C2)n1 ; Cc1ccc(S(=O)(=O)O)cc1] | 4-tert-butyl-2-(2,6- diazaspiro[3.3] heptan-2- ylmethyl)thiazole; 4- methylbenzene- sulfonic acid | tert-butyl 2,6- diazaspiro [3.3]heptane- 2- carboxylate hydrochloride; 4-tert- butylthiazole- 2- carbaldehyde (CAS: 211942-98- 2) | 252,2 [M − TsOH + H]⁺ |
| B.445 | 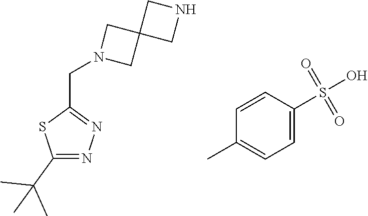 [SMILES: CC(C)(C)c1nnc(CN2CC3(CNC3)C2)s1 ; Cc1ccc(S(=O)(=O)O)cc1] | 2-tert-butyl-5-(2,6- diazaspiro[3.3]heptan- 2-ylmethyl)-1,3,4- thiadiazole; 4- methylbenzene- sulfonic acid | tert-butyl 2,6- diazaspiro [3.3]heptane- 2- carboxylate oxalate; 5- tert-butyl- 1,3,4- thiadiazole- 2- carbaldehyde (CAS: 2059955- 08-5) | 253.0 [M − TsOH + H]⁺ |
| B.446 | 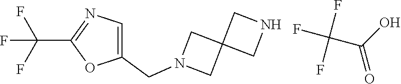 | 5-(2,6- diazaspiro[3.3]heptan- 2-ylmethyl)-2- (trifluoromethyl) oxazole; 2,2,2- trifluoroacetic acid | tert-butyl 2,6- diazaspiro [3.3]heptane- 2- carboxylate hydrochloride; 2- (trifluoro- methyl)oxazole- 5- carbaldehyde (CAS: 1378579- 95-3) | 248.2 [M − TFA + H]⁺ |
| B.447 | 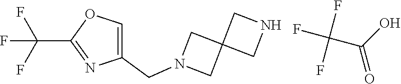 | 4-(2,6- diazaspiro[3.3] heptan- 2-ylmethyl)-2- (trifluoromethyl) oxazole; 2,2,2- trifluoroacetic acid | tert-butyl 2,6- diazaspiro [3.3]heptane- 2- carboxylate hydrochloride; 2- (trifluoro- methyl)oxazole- 4- carbaldehyde (CAS: 1240598- 47-3) | 248.2 [M − TFA + H]⁺ |
| B.448 | 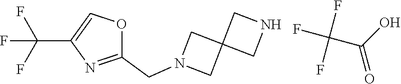 | 2-(2,6- diazaspiro[3.3]heptan- 2-ylmethyl)-4- (trifluoromethyl) oxazole; 2,2,2- trifluoroacetic acid | tert-butyl 2,6- diazaspiro [3.3]heptane- 2- carboxylate hydrochloride; 4- (trifluoro- methyl)oxazole- 2- carbaldehyde (CAS: 1784378- 54-6) | 248.1 [M − TFA + H]⁺ |
| B.449 | 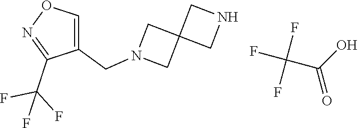 | 4-(2,6- diazaspiro[3.3]heptan- 2-ylmethyl)-3- (trifluoromethyl) isoxazole; 2,2,2- trifluoroacetic acid | tert-butyl 2,6- diazaspiro [3.3]heptane- 2- carboxylate hydrochloride; 3- (trifluoro- methyl) isoxazole-4- carbaldehyde (CAS: 1537457- 44-5) | 248.2 [M − TFA + H]⁺ |
| B.450 | 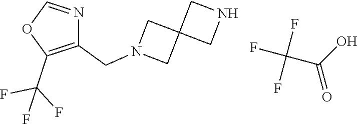 | 4-(2,6- diazaspiro[3.3]heptan- 2-ylmethyl)-5- (trifluoromethyl) oxazole; 2,2,2- trifluoroacetic acid | tert-butyl 2,6- diazaspiro [3.3]heptane- 2- carboxylate hydrochloride; 5- (trifluoro- methyl)oxazole- 4- carbaldehyde (CAS: 1781643- 66-0) | 248.1 [M − TFA + H]⁺ |
| B.451 | 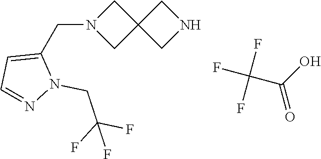 [SMILES: FC(F)(F)Cn1nccc1CN1CC2(CNC2)C1 ; O=C(O)C(F)(F)F] | 2-[[2-(2,2,2- trifluoroethyl)pyrazol- 3-yl]methyl]-2,6- diazaspiro[3.3] heptane; 2,2,2- trifluoroacetic acid | tert-butyl 2,6- diazaspiro [3.3]heptane- 2- carboxylate hydrochloride; 2-(2,2,2- trifluoroethyl) pyrazole- 3- carbaldehyde (CAS: 1227685- 53-1) | 261.2 [M − TFA + H]⁺ |
| B.452 | 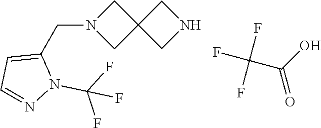 | 2-[[2- (trifluoromethyl) pyrazol-3-yl]methyl]- 2,6- diazaspiro[3.3] heptane; 2,2,2- trifluoroacetic acid | tert-butyl 2,6- diazaspiro [3.3]heptane- 2- carboxylate hydrochloride; 2- (trifluoro- methyl) pyrazole-3- carbaldehyde (CAS: 2091369- 84-3) | 247.0 [M − TFA + H]⁺ |
| B.453 | 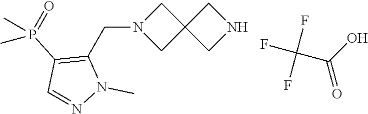 | 2-[(4- dimethylphosphoryl- 2-methyl-pyrazol-3- yl)methyl]-2,6- diazaspiro[3.3] heptane; 2,2,2- trifluoroacetic acid | tert-butyl 2- aza-6- azoniaspiro [3.3]heptane- 2- carboxylate oxalate; 4- dimethyl- phosphoryl-2- methyl- pyrazole-3- carbaldehyde (C.13) | 269.0 [M − TFA + H]⁺ |
| B.455 | 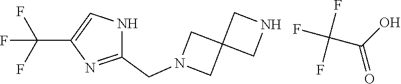 | 2-[[4- (trifluoromethyl)- 1H-imidazol-2- yl]methyl]-2,6- diazaspiro[3.3] heptane; 2,2,2- trifluoroacetic acid | 5- (trifluoro- methyl)-1H- imidazole- 2- carbaldehyde (CAS: 102808-02- 6) | 247.0 [M − TFA + H]⁺ |
| B.457 | 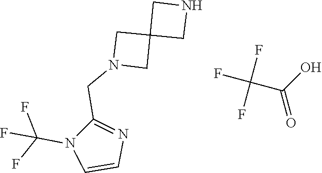 [SMILES: FC(F)(F)n1ccnc1CN1CC2(CNC2)C1 ; O=C(O)C(F)(F)F] | 2-[[1- (trifluoromethyl) imidazol-2-yl] methyl]- 2,6- diazaspiro[3.3] heptane; 2,2,2- trifluoroacetic acid; | tert-butyl 2,6- diazaspiro [3.3]heptane- 2- carboxylate hydrochloride; 1- (trifluoro- methyl) imidazole-2- carbaldehyde (CAS: 1554452- 65-1) | 247.2 [M − TFA + H]⁺ |
| B.458 | 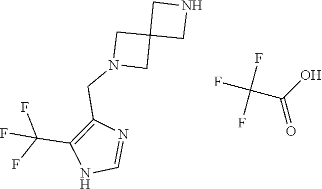 [SMILES: FC(F)(F)c1[nH]cnc1CN1CC2(CNC2)C1 ; O=C(O)C(F)(F)F] | 2-[[5- (trifluoromethyl)- 1H-imidazol-4- yl]methyl]-2,6- diazaspiro[3.3] heptane; 2,2,2- trifluoroacetic acid | tert-butyl 2,6- diazaspiro [3.3]heptane- 2- carboxylate hydrochloride; 5- (trifluoro- methyl)-1H- imidazole- 4- carbaldehyde (CAS: 915369-31- 2) | 247.2 [M − TFA + H]⁺ |
| B.460 | 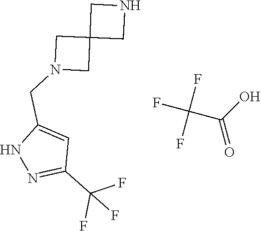 [SMILES: FC(F)(F)c1cc(CN2CC3(CNC3)C2)[nH]n1 ; O=C(O)C(F)(F)F] | 2-[[3- (trifluoromethyl)- 1H-pyrazol-5- yl]methyl]-2,6- diazaspiro[3.3] heptane; 2,2,2- trifluoroacetic acid | tert-butyl 2,6- diazaspiro [3.3]heptane- 2- carboxylate hydrochloride; 5- (trifluoro- methyl)-1H- pyrazole-3- carbaldehyde (CAS: 91234-14-9) | 247.0 [M − TFA + H]⁺ |
| B.461 | 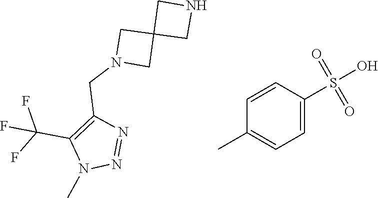 [SMILES: Cn1nnc(CN2CC3(CNC3)C2)c1C(F)(F)F ; Cc1ccc(S(=O)(=O)O)cc1] | 2-[1-methyl-5- (trifluoromethyl) triazol-4-yl] methyl]-2,6- diazaspiro[3.3] heptane; 4- methylbenzene- sulfonic acid | tert-butyl 2,6- diazaspiro [3.3]heptane- 2- carboxylate hydrochloride; 1- methyl-5- (trifluoro- methyl)triazole- 4- carbaldehyde (CAS: 1267185- 70-5) | 262.0 [M − TsOH + H]⁺ |
| B.462 | 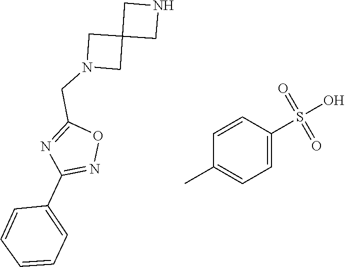 [SMILES: c1ccc(-c2noc(CN3CC4(CNC4)C3)n2)cc1 ; Cc1ccc(S(=O)(=O)O)cc1] | 5-(2,6- diazaspiro[3.3]heptan- 2-ylmethyl)-3- phenyl-1,2,4- oxadiazole; 4- methylbenzene- sulfonic acid | 2,6- diazaspiro [3.3]heptane- 2- carboxylic acid tert- butyl ester oxalate; 3- phenyl- 1,2,4- oxadiazole- 5- carbaldehyde (CAS: 73217-75-1) | 257.1 [M − TsOH + H]⁺ |
| B.465 | 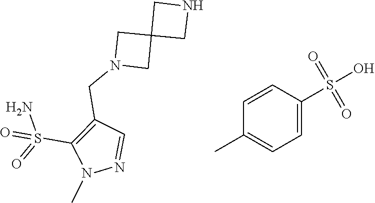 [SMILES: Cn1ncc(CN2CC3(CNC3)C2)c1S(N)(=O)=O ; Cc1ccc(S(=O)(=O)O)cc1] | 4-(2,6- diazaspiro[3.3]heptan- 2-ylmethyl)-2- methyl-pyrazole-3- sulfonamide; 4- methylbenzene- sulfonic acid | tert-butyl 2,6- diazaspiro [3.3]heptane- 2- carboxylate hydrochloride; 4- formyl-2- methyl- pyrazole-3- sulfonamide (CAS: 121905-03- 1) | 272.2 [M − TsOH + H]⁺ |
| B.466 | 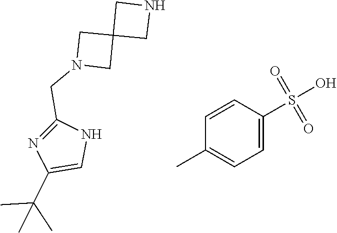 [SMILES: CC(C)(C)c1c[nH]c(CN2CC3(CNC3)C2)n1 ; Cc1ccc(S(=O)(=O)O)cc1] | 2-[(4-tert-butyl-1H- imidazol-2- yl)methyl]-2,6- diazaspiro[3.3] heptane; 4- methylbenzene- sulfonic acid | tert-butyl 2- aza-6- azoniaspiro [3.3]heptane- 2- carboxylate oxalate; 4- tert-butyl- 1H- imidazole- 2- carbaldehyde (CAS: 1339064- 78-6) | 235.2 [M − TsOH + H]⁺ |
| B.468 | 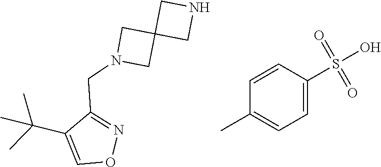 [SMILES: CC(C)(C)c1conc1CN1CC2(CNC2)C1 ; Cc1ccc(S(=O)(=O)O)cc1] | 4-tert-butyl-3-(2,6- diazaspiro[3.3] heptan-2- ylmethyl)isoxazole; 4- methylbenzene- sulfonic acid | tert-butyl 2- aza-6- azoniaspiro [3.3]heptane- 2- carboxylate oxalate; 4- tert- butylisoxazole- 3- carbaldehyde (CAS: 2416243- 20-2) | 236.0 [M − TsOH + H]⁺ |
| B.469 | 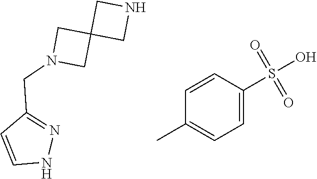 | 2-(1H-pyrazol-3- ylmethyl)-2,6- diazaspiro[3.3] heptane; 4- methylbenzene- sulfonic acid; | tert-butyl 2,6- diazaspiro [3.3]heptane- 2- carboxylate hydrochloride; 1H- pyrazole-3- carbaldehyde (CAS: 3920-50-1) | 179.2 [M − TsOH + H]⁺ |
| B.471 | 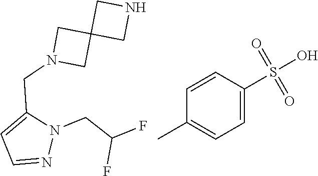 | 2-[[2-(2,2- difluoroethyl)pyrazol- 3-yl]methyl]-2,6- diazaspiro[3.3] heptane; 4- methylbenzene- sulfonic acid | tert-butyl 2,6- diazaspiro [3.3]heptane- 2- carboxylate hydrochloride; 2-(2,2- difluoroethyl) pyrazole- 3- carbaldehyde (CAS: 1783782- 61-5) | 243.2 [M − TsOH + H]⁺ |
| B.472 | 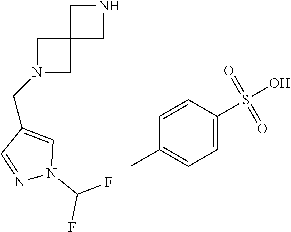 | 2-[[1- (difluoromethyl) pyrazol- 4-yl]methyl]-2,6- diazaspiro[3.3] heptane; 4- methylbenzene- sulfonic acid | tert-butyl 2,6- diazaspiro [3.3]heptane- 2- carboxylate hydrochloride; 1- (difluoromethyl) pyrazole-4- carbaldehyde (CAS: 1216244- 84-6) | 229,2 [M − TsOH + H]⁺ |
| B.474 | 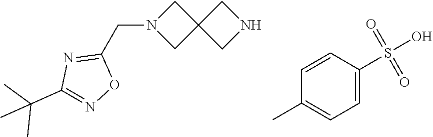 [SMILES: CC(C)(C)c1noc(CN2CC3(CNC3)C2)n1 ; Cc1ccc(S(=O)(=O)O)cc1] | 3-tert-butyl-5-(2,6- diazaspiro[3.3]heptan- 2-ylmethyl)-1,2,4- oxadiazole; 4- methylbenzene- sulfonic acid | tert-butyl 2,6- diazaspiro[3.3] heptane- 2- carboxylate oxalate; 3- tert-butyl- 1,2,4- oxadiazole- 5- carbaldehyde (CAS: 944901-78- 4) | 237.2 [M − TsOH + H]⁺ |
| B.475 | 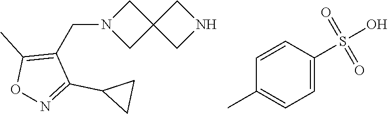 [SMILES: Cc1onc(C2CC2)c1CN1CC2(CNC2)C1 ; Cc1ccc(S(=O)(=O)O)cc1] | 3-cyclopropyl-4- (2,6- diazaspiro[3.3]heptan- 2-ylmethyl)-5- methyl-isoxazole; 4- methylbenzene- sulfonic acid | tert-butyl 2,6- diazaspiro[3.3] heptane- 2- carboxylate hydrochloride; 3- cyclopropyl- 5-methyl- isoxazole-4- carbaldehyde (CAS: 878204-45- 6 | 234.2 [M − TsOH + H]⁺ |
| B.476 | 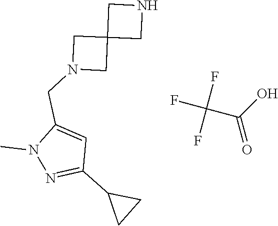 [SMILES: Cn1nc(C2CC2)cc1CN1CC2(CNC2)C1 ; O=C(O)C(F)(F)F] | 2-[(5-cyclopropyl-2- methyl-pyrazol-3- yl)methyl]-2,6- diazaspiro[3.3] heptane; 2,2,2- trifluoroacetic acid | tert-butyl 2,6- diazaspiro [3.3]heptane- 2- carboxylate hydrochloride; 3- cyclopropyl- 1-methyl- 1H- pyrazole-5- carbaldehyde (CAS: 1245808- 37-0) | 233.2 [M − TFA + H]⁺ |
| B.477 | 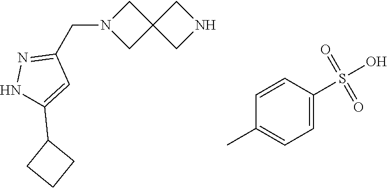 [SMILES: c1c(CN2CC3(CNC3)C2)n[nH]c1C1CCC1 ; Cc1ccc(S(=O)(=O)O)cc1] | 2-[(5-cyclobutyl-1H- pyrazol-3- yl)methyl]-2,6- diazaspiro[3.3] heptane; 4- methylbenzene- sulfonic acid | tert-butyl 2,6 diazaspiro [3.3]heptane- 2- carboxylate hydrochloride; 5- cyclobutyl- 1H- pyrazole-3- carbaldehyde (CAS: 1779480- 21-5) | 234.2 [M − TsOH + H]⁺ |
| B.479 | 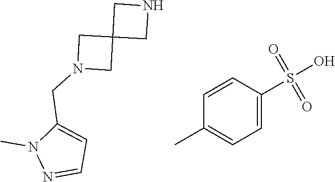 | 2-[(2-methylpyrazol- 3-yl)methyl]-2,6- diazaspiro[3.3] heptane; 4- methylbenzene- sulfonic acid | tert-butyl 2,6- diazaspiro [3.3]heptane- 2- carboxylate oxalate; 2- methyl- pyrazole-3- carbaldehyde (CAS: 27258-33-9) | 193.1 [M − TsOH + H]⁺ |
| B.480 | 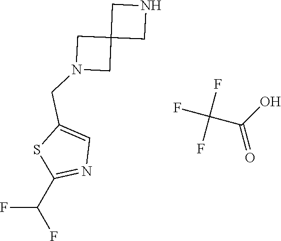 [SMILES: FC(F)c1ncc(CN2CC3(CNC3)C2)s1 ; O=C(O)C(F)(F)F] | 5-(2,6- diazaspiro[3.3]heptan- 2-ylmethyl)-2- (difluoromethyl) thiazole; 2,2,2- trifluoroacetic acid | tert-butyl 2,6- diazaspiro [3.3]heptane- 2- carboxylate hydrochloride; 2- (difluoro- methyl) thiazole-5- carbaldehyde (CAS: 1779840- 82-2) | 246.2 [M − TFA + H]⁺ |
| B.481 | 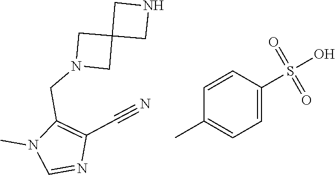 | 5-(2,6- diazaspiro[3.3]heptan- 2-ylmethyl)-1- methyl-imidazole-4- carbonitrile; 4- methylbenzene- sulfonic acid | tert-butyl 2,6- diazaspiro [3.3]heptane- 2- carboxylate oxalate; 5- formyl-1- methyl- imidazole- 4- carbonitrile (CAS: 916257-35- 7) | 218.0 [M − TsOH + H]⁺ |
| B.482 | 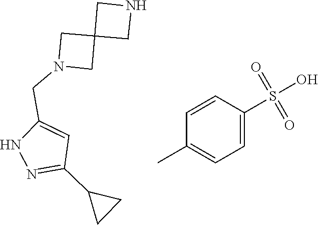 [SMILES: c1c(C2CC2)n[nH]c1CN1CC2(CNC2)C1 ; Cc1ccc(S(=O)(=O)O)cc1] | 2-[(3-cyclopropyl- 1H-pyrazol-5- yl)methyl]-2,6- diazaspiro[3.3] heptane; 4- methylbenzene sulfonic acid | tert-butyl 2,6- diazaspiro [3.3]heptane- 2- carboxylate oxalate; 3- cyclopropyl- 1H- pyrazole-5- carbaldehyde (CAS: 1284220- 47-8) | 219.1 [M − TsOH + H]⁺ |
| B.484 | 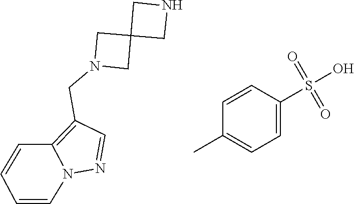 [SMILES: c1ccn2ncc(CN3CC4(CNC4)C3)c2c1 ; Cc1ccc(S(=O)(=O)O)cc1] | 3-(2,6- diazaspiro[3.3] heptan-2- ylmethyl)pyrazolo[1, 5-a]pyridine; 4- methylbenzene- sulfonic acid | 2,6- diazaspiro [3.3]heptane- 2- carboxylic acid tert- butyl ester; pyrazolo[1, 5-a]pyridine- 3- carbaldehyde (CAS : 73957-66-1) | 229.1 [M − TsOH + H]⁺ |
| B.489 | 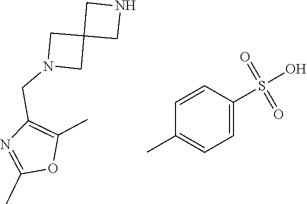 | 4-(2,6- diazaspiro[3.3]heptan- 2-ylmethyl)-2,5- dimethyl-oxazole; 4- methylbenzene- sulfonic acid | tert-butyl 2,6- diazaspiro [3.3]heptane- 2- carboxylate hydrochloride; 2,4- dimethyl- oxazole-5- carbaldehyde (CAS: 69062-86-8) | 208.1 [M- TsOH+ H]+ |
| B.490 | 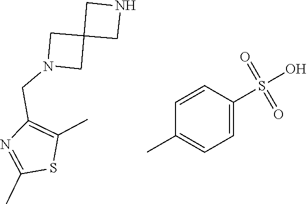 | 4-(2,6- diazaspiro[3.3]heptan- 2-ylmethyl)-2,5- dimethyl-thiazole; 4- methylbenzene- sulfonic acid | 2,6- diazaspiro[3.3] heptane- 2- carboxylic acid tert- butyl ester; 2,4- dimethyl- 1,3- thiazole-5- carbaldehyde (CAS: 95453-54-6) | 224.1 [M − TsOH + H]⁺ |
| B.515 | 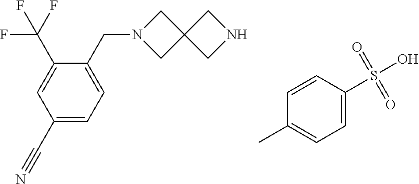 [SMILES: N#Cc1ccc(CN2CC3(CNC3)C2)c(C(F)(F)F)c1 ; Cc1ccc(S(=O)(=O)O)cc1] | 4-(2,6- diazaspiro[3.3]heptan- 2-ylmethyl)-3- (trifluoromethyl) benzonitrile; 4- methylbenzene- sulfonic acid | 2,6- diazaspiro [3.3]heptane- 2- carboxylic acid tert- butyl ester; 4-formyl-3- (trifluoro- methyl) benzonitrile (CAS: 211796-64- 4) | 282.1 [M − TsOH + H]⁺ |
| B.516 | 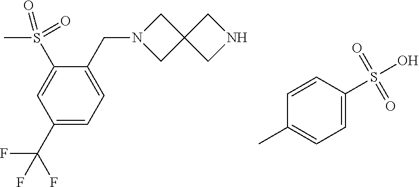 [SMILES: CS(=O)(=O)c1cc(C(F)(F)F)ccc1CN1CC2(CNC2)C1 ; Cc1ccc(S(=O)(=O)O)cc1] | 2-[[2- methylsulfonyl-4- (trifluoromethyl) phenyl]methyl]-2,6- diazaspiro[3.3] heptane; 4- methylbenzene- sulfonic acid | 2,6- diazaspiro [3.3]heptane- 2- carboxylic acid tert- butyl ester; 2-mesyl-4- (trifluoro- methyl) benzaldehyde (CAS: 225502-25- 0) | 335.1 [M − TsOH + H]⁺ |
| B.517 | 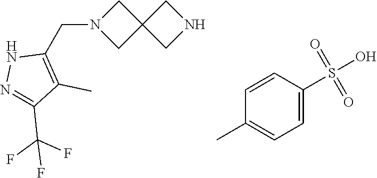 [SMILES: Cc1c(C(F)(F)F)n[nH]c1CN1CC2(CNC2)C1 ; Cc1ccc(S(=O)(=O)O)cc1] | 2-[[4-methyl-3- (trifluoromethyl)- 1H-pyrazol-5- yl]methyl]-2,6- diazaspiro[3.3] heptane; 4- methylbenzene- sulfonic acid | 2,6- diazaspiro [3.3]heptane- 2- carboxylic acid tert- butyl ester; 4-methyl-3- (trifluoro- methyl)-1H- pyrazole-5- carbaldehyde (CAS: 1284219- 94-8) | 259.2 [M − TsOH + H]⁺ |
| B.518 | 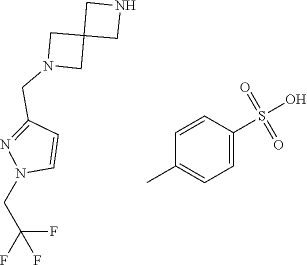 [SMILES: FC(F)(F)Cn1ccc(CN2CC3(CNC3)C2)n1 ; Cc1ccc(S(=O)(=O)O)cc1] | 2-[1-(2,2,2- trifluoroethyl)pyrazol- 3-yl]methyl]-2,6- diazaspiro[3.3] heptane; 4- methylbenzene- sulfonic acid | 2,6- diazaspiro [3.3]heptane- 2- carboxylic acid tert- butyl ester; 1-(3,3,3- trifluoro- propyl) pyrazole-3- carbaldehyde (C.22) | 275.1 [M − TsOH + H]⁺ |
| B.519 | 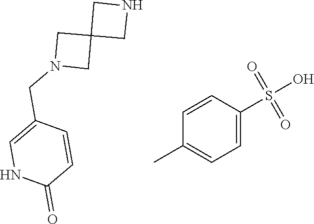 | 5-(2,6- diazaspiro[3.3]heptan- 2-ylmethyl)-1H- pyridin-2-one; 4- methylbenzene- sulfonic acid | 2,6- diazaspiro [3.3]heptane- 2- carboxylic acid tert- butyl ester; 6-oxo-1H- pyridine-3- carbaldehyde (CAS: 106984-91- 2) | 206.0 [M − TsOH + H]⁺ |
| B.522 | 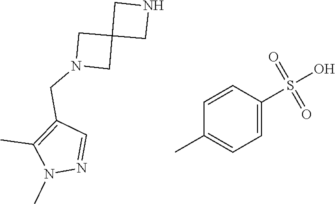 | 2-[(1,5- dimethylpyrazol-4- yl)methyl]-2,6- diazaspiro[3.3] heptane; 4- methylbenzene- sulfonic acid | 2,6- diazaspiro [3.3]heptane- 2- carboxylic acid tert- butyl ester hydrochloride; 1,5- dimethyl- pyrazole-4- carbaldehyde (CAS: 25711-30-2) | 207.2 [M − TsOH + H]⁺ |
| B.523 | 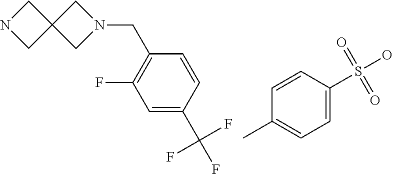 | 2-[[2-fluoro-4- (trifluoromethyl) phenyl] methyl]-2,6- diazaspiro[3.3] heptane; 4- methylbenzene- sulfonic acid | 2,6- diazaspiro [3.3]heptane- 2- carboxylic acid tert- butyl ester; oxalic acid; 2- fluoro-4- (trifluoro- methyl) benzaldehyde | 275.2 [M − TsOH + H]⁺ |
| B.524 | 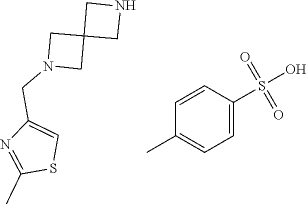 | 4-(2,6- diazaspiro[3.3]heptan- 2-ylmethyl)-2- methyl-thiazole; 4- methylbenzene- sulfonic acid | 2,6- diazaspiro [3.3]heptane- 2- carboxylic acid tert- butyl ester; formic acid; 2- methylthiazole- 4- carbaldehyde (CAS: 20949-84-2) | 210.1 [M − TsOH + H]⁺ |

### Example B.55

### 2-[3-(trifluoromethoxy)phenyl]sulfonyl]-2,6-diazaspiro[3.3]heptane; Trifluoroacetic

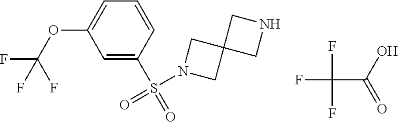 [SMILES: O=S(=O)(c1cccc(OC(F)(F)F)c1)N1CC2(CNC2)C1 ; O=C(O)C(F)(F)F]

To a solution of 2-[3-(trifluoromethoxy)phenyl]sulfonyl-2,6-diazaspiro[3.3]heptane-6-carboxylic acid tert-butyl ester (1350 mg, 3.2 mmol) in dichloromethane (13.5 mL) was added TFA (3.64 g, 2.46 mL, 32.0 mmol) and the reaction mixture was stirred at room temperature for 18 h. Volatiles were removed in vacuo to yield 1855 mg of the crude title compound (purity roughly 70% major contaminant excess of TFA), which was used without further purification. MS (ESI): m/z=323.1 [M-TFA+H]⁺

### Step a) 2-[3-(trifluoromethoxy)phenyl]sulfonyl-2,6-diazaspiro[3.3]heptane-6-carboxylic Acid Tert-Butyl Ester

To a suspension of 2,6-diazaspiro[3.3]heptane-2-carboxylic acid tert-butyl ester (790 mg, 3.98 mmol) in dichloromethane (18 mL) cooled down to 0° C. was added DIPEA (773 mg, 1.04 mL, 5.98 mmol) and 3-(trifluoromethoxy)benzenesulfonyl chloride (1.04 g, 3.98 mmol) after which the reaction mixture was stirred at 0° C. for 10 min and at room temp. for 1 h. The reaction mixture was diluted with dichloromethane and extracted with aq. Na₂CO₃ 1M solution. The organic phase was collected and the aqueous phase was back-extracted with dichloromethane. The combined organic phases were dried over sodium sulfate and evaporated down to dryness. The residue was purified by FC (SiO₂; heptane/EtOAc) to yield 1350 mg of the title compound. MS (ESI): m/z=367.1 [M-tBu+H]⁺

In analogy to Example B.55, the following building blocks were generated using the relevant building blocks in Step a). For Examples B.167 and B.168, 2,7-diazaspiro[3.5]nonane-2-carboxylic acid tert-butyl ester was used in place of 2,6-diazaspiro[3.3]heptane-2-carboxylic acid tert-butyl ester.

| | | | | MS, |
|---|---|---|---|---|
| | | | Building | ESI: |
| Ex. | Structure | Systematic Name | Blocks | m/z |
| B.56 | 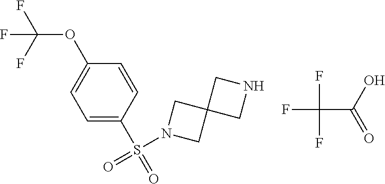 | 2-[4- (trifluoromethoxy) phenyl]sulfonyl-2,6- diazaspiro[3.3] heptane; trifluoroacetic acid | 4- (trifluoro- methoxy) benzene- sulfonyl chloride | 323.2 [M − TFA + H]⁺ |
| B.57 | 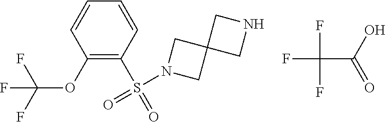 [SMILES: O=S(=O)(c1ccccc1OC(F)(F)F)N1CC2(CNC2)C1 ; O=C(O)C(F)(F)F] | 2-[2- (trifluoromethoxy) phenyl]sulfonyl-2,6- diazaspiro[3.3] heptane; trifluoroacetic acid | 2- (trifluoro- methoxy) benzene- sulfonyl chloride | 323.2 [M − TFA + H]⁺ |
| B.58 | 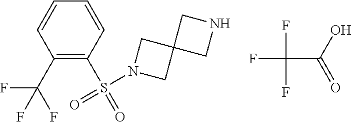 [SMILES: O=S(=O)(c1ccccc1C(F)(F)F)N1CC2(CNC2)C1 ; O=C(O)C(F)(F)F] | 2-[2- (trifluoromethyl) phenyl]sulfonyl-2,6- diazaspiro[3.3] heptane; trifluoroacetic acid | 2- (trifluoro- methyl) benzene- sulfonyl chloride | 307.2 [M − TFA + H]⁺ |
| B.59 | 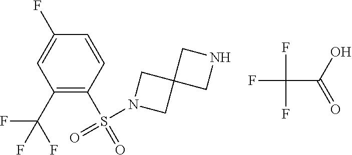 | 2-[4-fluoro-2- (trifluoromethyl) phenyl]sulfonyl-2,6- diazaspiro[3.3] heptane; trifluoroacetic acid | 4-fluoro-2- (trifluoro- methyl) benzene- sulfonyl chloride | 325.1 [M − TFA + H]⁺ |
| B.60 | 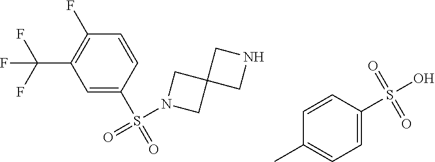 [SMILES: O=S(=O)(c1ccc(F)c(C(F)(F)F)c1)N1CC2(CNC2)C1 ; Cc1ccc(S(=O)(=O)O)cc1] | 2-[4-fluoro-3- (trifluoromethyl) phenyl]sulfonyl-2,6- diazaspiro[3.3] heptane; 4- methylbenzene- sulfonic acid | 4-fluoro-3- (trifluoro- methyl) benzene- sulfonyl chloride | 325.0 [M − TsOH + H]⁺ |
| B.61 | 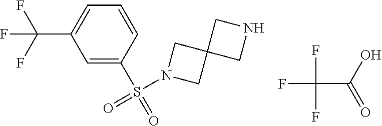 [SMILES: O=S(=O)(c1cccc(C(F)(F)F)c1)N1CC2(CNC2)C1 ; O=C(O)C(F)(F)F] | 2-[3- (trifluoromethyl) phenyl]sulfonyl-2,6- diazaspiro[3.3] heptane; trifluoroacetic acid | 3- (trifluoro- methyl) benzene- sulfonyl chloride | 307.1 [M − TFA + H]⁺ |
| B.62 | 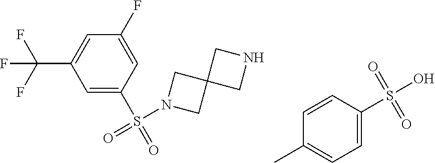 [SMILES: O=S(=O)(c1cc(F)cc(C(F)(F)F)c1)N1CC2(CNC2)C1 ; Cc1ccc(S(=O)(=O)O)cc1] | 2-[3-fluoro-5- (trifluoromethyl) phenyl]sulfonyl-2,6- diazaspiro[3.3] heptane; 4- methylbenzene- sulfonic acid | 3-fluoro-5- (trifluoro- methyl) benzene- sulfonyl chloride | 325.0 [M − TsOH + H]⁺ |
| B.63 | 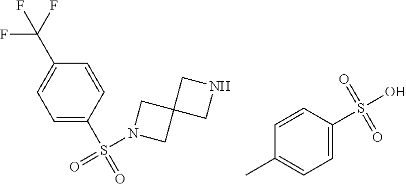 [SMILES: O=S(=O)(c1ccc(C(F)(F)F)cc1)N1CC2(CNC2)C1 ; Cc1ccc(S(=O)(=O)O)cc1] | 2-[4- (trifluoromethyl) phenyl]sulfonyl-2,6- diazaspiro[3.3] heptane; 4- methylbenzene- sulfonic acid | 4- (trifluoro- methyl) benzene- sulfonyl chloride | 307.0 [M − TsOH + H]⁺ |
| B.64 | 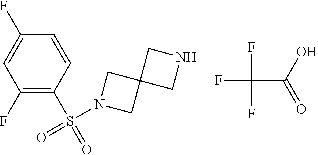 | 2-(2,4- difluorophenyl) sulfonyl-2,6- diazaspiro[3.3] heptane; trifluoroacetic acid | 2,4- difluoro- benzene- sulfonyl chloride | 275.1 [M − TFA + H]⁺ |
| B.65 | 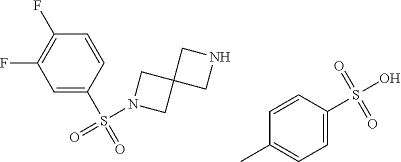 [SMILES: O=S(=O)(c1ccc(F)c(F)c1)N1CC2(CNC2)C1 ; Cc1ccc(S(=O)(=O)O)cc1] | 2-(3,4- difluorophenyl) sulfonyl-2,6- diazaspiro[3.3] heptane; 4- methylbenzene- sulfonic acid | 3,4- difluoro- benzene- sulfonyl chloride | 275.0 [M − TsOH + H]⁺ |
| B.66 | 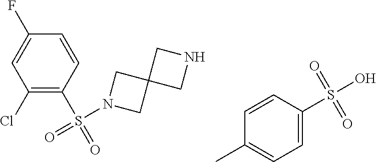 [SMILES: O=S(=O)(c1ccc(F)cc1Cl)N1CC2(CNC2)C1 ; Cc1ccc(S(=O)(=O)O)cc1] | 2-(2-chloro-4-fluoro- phenyl)sulfonyl-2,6- diazaspiro[3.3] heptane; 4- methylbenzene- sulfonic acid | 2-chloro-4- fluoro- benzene- sulfonyl chloride | 291.2 [M − TsOH + H]⁺ |
| B.67 | 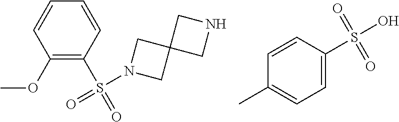 [SMILES: COc1ccccc1S(=O)(=O)N1CC2(CNC2)C1 ; Cc1ccc(S(=O)(=O)O)cc1] | 2-(2- methoxyphenyl) sulfonyl-2,6- diazaspiro[3.3] heptane; 4- methylbenzene- sulfonic acid | 2- methoxy- benzene- sulfonyl chloride | 269.2 [M − TsOH + H]⁺ |
| B.68 | 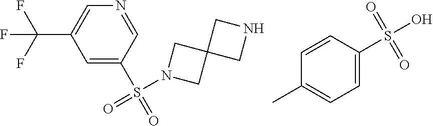 [SMILES: O=S(=O)(c1cncc(C(F)(F)F)c1)N1CC2(CNC2)C1 ; Cc1ccc(S(=O)(=O)O)cc1] | 2-[[5- (trifluoromethyl)-3- pyridyl]sulfonyl]- 2,6- diazaspiro[3.3] heptane; 4- methylbenzene- sulfonic acid | 5- (trifluoro- methyl) pyridine- 3-sulfonyl chloride (CAS: 1060802- 03-0) | 308.0 [M − TsOH + H]⁺ |
| B.33 | 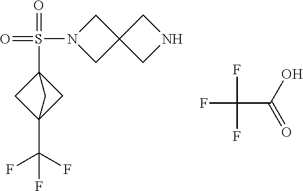 [SMILES: O=S(=O)(N1CC2(CNC2)C1)C12CC(C(F)(F)F)(C1)C2 ; O=C(O)C(F)(F)F] | 2-[[3- (trifluoromethyl)-1- bicyclo[1.1.1] pentanyl] sulfonyl]-2,6- diazaspiro[3.3] heptane; trifluoroacetic acid | 3- (trifluoro- methyl) bicyclo [1.1.1] pentane-1- sulfonyl chloride (CAS: 2167111- 82-0) | 297.1 [M − TFA + H]⁺ |
| B.148 | 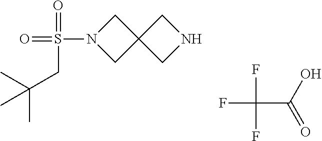 | 2-(2,2- dimethylpropyl- sulfonyl)-2,6- diazaspiro[3.3] heptane; trifluoroacetic acid | 2,2- dimethyl- propane-1- sulfonyl chloride | 233.2 [M − TFA + H]⁺ |
| B.167 | 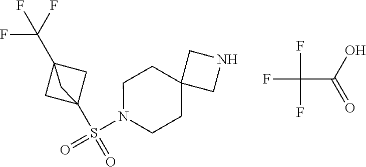 [SMILES: O=S(=O)(N1CCC2(CC1)CNC2)C12CC(C(F)(F)F)(C1)C2 ; O=C(O)C(F)(F)F] | 7-[[3- (trifluoromethyl)-1- bicyclo[1.1.1] pentanyl] sulfonyl]-2,7- diazaspiro[3.5] nonane; trifluoroacetic acid | 3- (trifluoro- methyl) bicyclo [1.1.1] pentane-1- sulfonyl chloride | 325.1 [M − TFA + H]⁺ |
| B.168 | 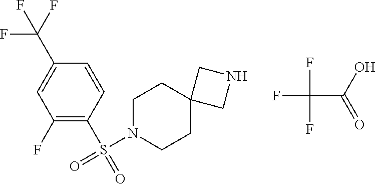 [SMILES: O=S(=O)(c1ccc(C(F)(F)F)cc1F)N1CCC2(CC1)CNC2 ; O=C(O)C(F)(F)F] | 7-[2-fluoro-4- (trifluoromethyl) phenyl]sulfonyl-2,7- diazaspiro[3.5] nonane; 2,2,2- trifluoroacetic acid | 2-fluoro-4- (trifluoro- methyl) benzene- sulfonyl chloride | 353.1 [M − TFA + H]⁺ |
| B.262 | 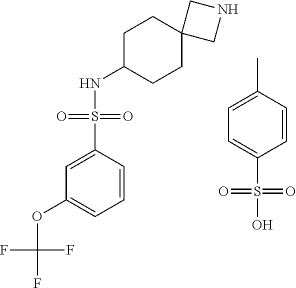 | N-(2- azaspiro[3.5]nonan- 7-yl)-3- (trifluoromethoxy) benzenesulfonamide; 4- methylbenzene- sulfonic acid | 7-amino-2- azaspiro[3.5] nonane-2- carboxylic acid tert- butyl ester (CAS RN: 1408075- 19-3) and 3- (trifluoro- methoxy) benzene- sulfonyl chloride (CAS RN: 220227-84- 9) | 409.1 [M − tBu + H]⁺ |

### Example B.83

### 6-((6-(trifluoromethyl)pyridin-3 yl)oxy)-2-azaspiro[3.3]heptane; 4-methylbenzenesulfonic Acid

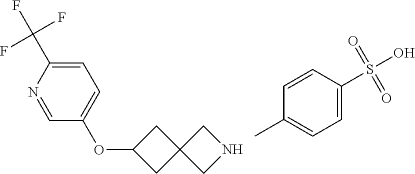

To a solution of tert-butyl 6-((6-(trifluoromethyl)pyridin-3-yl)oxy)-2-azaspiro[3.3]heptane-2-carboxylate (1.85 g, 5.16 mmol) in ethyl acetate (12.3 mL) was added 4-methylbenzenesulfonic acid monohydrate (1.03 g, 5.42 mmol). Then, the reaction mixture was refluxed (80° C.) for 16 h. The reaction mixture was cooled and the solvent was evaporated to give the title compound (2.25 g, 96% yield) as a light brown solid (2.25 g, 96%). MS (ESI): m/z=259.2 [M−TsOH+H]⁺

### Step a) Tert-Butyl 6-((6-(trifluoromethyl)pyridin-3-yl)oxy)-2-azaspiro[3.3]heptane-2-carboxylate

To a solution of 6-(trifluoromethyl)pyridin-3-ol (1.0 g, 6.13 mmol) in dry DMF (24.5 mL) was added NaH (270 mg, 6.74 mmol). The reaction mixture was stirred at RT for 30 min, followed by addition of tert-butyl 6-((methylsulfonyl)oxy)-2-azaspiro[3.3]heptane-2-carboxylate (CAS: 1239320-11-6) (1.79 g, 6.13 mmol). The reaction mixture was stirred at 80° C. for 16 h. The reaction mixture was diluted with EtOAc and washed with 1 M aq. NaHCO₃ solution. The organic phase was collected and the aqueous phase underwent back-extraction with EtOAc. The combined organic layers were dried over Na₂SO₄ and evaporated to dryness to give the title compound (1.95 g, 89°yield), which was obtained as a light brown solid. MS (ESI): m/z=303.1 [M-tBu+H]⁺

### Example B.101

### 2-[[6-(trifluoromethyl)pyridazin-3-yl]methyl]-2,6-diazaspiro[3.3]heptane; di 4-methylbenzenesulfonic Acid

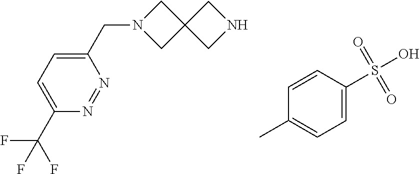 [SMILES: FC(F)(F)c1ccc(CN2CC3(CNC3)C2)nn1 ; Cc1ccc(S(=O)(=O)O)cc1]

A mixture of p-toluenesulfonic acid (1010 mg, 5.86 mmol), tert-butyl 6-[[6-(trifluoromethyl)pyridazin-3-yl]methyl]-2,6-diazaspiro[3.3]heptane-2-carboxylate (1000 mg, 2.79 mmol) in EtOAc (10 mL) was stirred at 80° C. for 12 h. The mixture was filtered and cake was concentrated to give the title compound (1450 mg, 86% yield). MS (ESI): m/z=59.2 [M−2TsOH+H]⁺

### Step a) 3-(chloromethyl)-6-(trifluoromethyl)pyridazine

A mixture of 3-methyl-6-(trifluoromethyl)pyridazine (2.0 g, 12.3 mmol) in 1,2-dichloroethane (40 mL) was added trichloroisocyanuric acid (958 mg, 4.12 mmol). The mixture was heated to 80° C. and stirred for 12 h. The residue was purified by FC to give the title compound (1.3 g, 54% yield) as a white solid. MS (ESI): m/z=197.1 [M+H]⁺

### Step b) tert-butyl 6-[[6-(trifluoromethyl)pyridazin-3 yl]methyl]-2,6-diazaspiro[3.3]heptane-2-carboxylate

To a solution of 3-(chloromethyl)-6-(trifluoromethyl)pyridazine (1.3 g, 6.61 mmol), ten-butyl 2,6-diazaspiro[3.3]heptane-2-carboxylate; oxalic acid (3.22 g, 6.61 mmol) in ACN (10 mL) was added K₂CO₃ (1.83 g, 13.2 mmol) at 25° C. The mixture was stirred at 25° C. for 12 h. The mixture was stirred at 50° C. for 2 h. The residue was purified by silica column (petroleum ether:ethyl acetate=10:1 to 0:1) and concentrated under reduced pressure to give the title compound (1.7 g, 71.7% yield) as a white solid. MS (ESI): m/z=359.3 [M+H]⁺

In analogy to Example B. 101, the following building blocks were generated using the relevant commercial building blocks in Step b).

| | | | | MS, |
|---|---|---|---|---|
| | | | Building | ESI: |
| Ex. | Structure | Systematic Name | Blocks | m/z |
| B.103 | 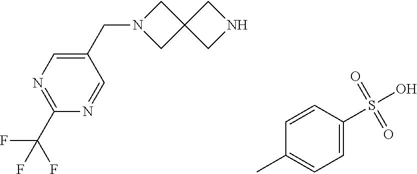 [SMILES: FC(F)(F)c1ncc(CN2CC3(CNC3)C2)cn1 ; Cc1ccc(S(=O)(=O)O)cc1] | 2-[[2- (trifluoromethyl) pyrimidin- 5-yl]methyl]- 2,6- diazaspiro[3.3] heptane; 4- methylbenzene- sulfonic acid | 5- (bromo- methyl)-2- (trifluoro- methyl) pyrimidine | 259.1 [M + H]⁺ |
| B.105 | 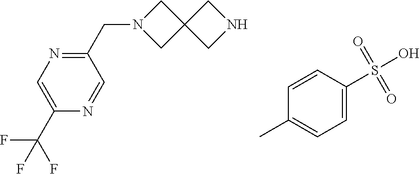 [SMILES: FC(F)(F)c1cnc(CN2CC3(CNC3)C2)cn1 ; Cc1ccc(S(=O)(=O)O)cc1] | 2-[[5- (trifluoromethyl) pyrazin-2-yl] methyl]-2,6- diazaspiro[3.3] heptane; 4- methylbenzene- sulfonic acid | 2- (bromo- methyl)-5- (trifluoro- methyl) pyrazine (CAS: 1447914- 67-1) | 259.4 [M + H]⁺ |
| B.111 | 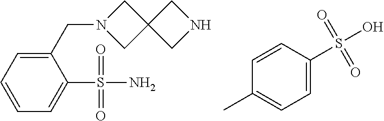 [SMILES: NS(=O)(=O)c1ccccc1CN1CC2(CNC2)C1 ; Cc1ccc(S(=O)(=O)O)cc1] | 2-(2,6- diazaspiro[3.3] heptan-2- ylmethyl)benzene- sulfonamide; 4- methylbenzene- sulfonic acid | 2- (chloro- methyl) benzene-1- sulfonamide (CAS: 81629-77-8) | 266.2 [M + H]⁺ |
| B.112 | 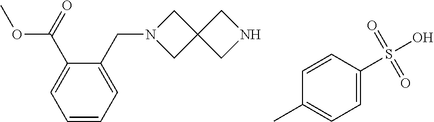 [SMILES: COC(=O)c1ccccc1CN1CC2(CNC2)C1 ; Cc1ccc(S(=O)(=O)O)cc1] | methyl 2-(2,6- diazaspiro[3.3] heptan-2- ylmethyl)benzoate; 4- methylbenzene- sulfonic acid | methyl 2- (bromo- methyl) benzoate | 247.2 [M + H]⁺ |
| B.323 | 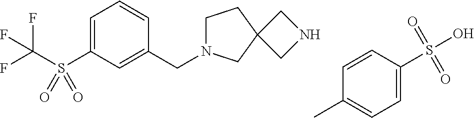 [SMILES: O=S(=O)(c1cccc(CN2CCC3(CNC3)C2)c1)C(F)(F)F ; Cc1ccc(S(=O)(=O)O)cc1] | 6-[[3- (trifluoromethyl- sulfonyl)phenyl] methyl]-2,6- diazaspiro[3.4] octane; 4- methylbenzene- sulfonic acid | 1- (bromo- methyl)-3- (trifluoro- methyl- sulfonyl) benzene (CAS: 45767-66-6) | 335.0 [M + H]⁺ |
| B.327 | 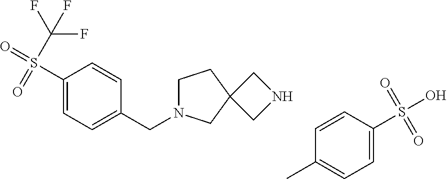 [SMILES: O=S(=O)(c1ccc(CN2CCC3(CNC3)C2)cc1)C(F)(F)F ; Cc1ccc(S(=O)(=O)O)cc1] | 6-[[4- (trifluoromethyl- sulfonyl)phenyl] methyl]-2,6- diazaspiro[3.4] octane; 4- methylbenzene- sulfonic acid | 1- (bromo- methyl)-4- (trifluoro- methyl- sulfonyl) benzene (CAS: 118089-89- 7) | 335.1 [M + H]⁺ |
| B.360 | 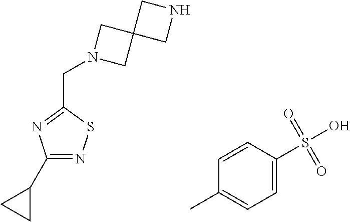 [SMILES: C1CC1c1nsc(CN2CC3(CNC3)C2)n1 ; Cc1ccc(S(=O)(=O)O)cc1] | 3-cyclopropyl-5- (2,6- diazaspiro[3.3] heptan- 2-ylmethyl)-1,2,4- thiadiazole; 4- methylbenzene- sulfonic acid | 5- (chloro- methyl)-3- cyclopropyl- 1,2,4- thiadiazole (CAS: 2090447- 99-5) | 237.0 [M + H]⁺ |
| B.361 | 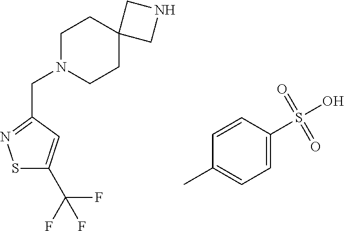 [SMILES: FC(F)(F)c1cc(CN2CCC3(CC2)CNC3)ns1 ; Cc1ccc(S(=O)(=O)O)cc1] | 3-(2,7- diazaspiro[3.5] nonan- 7-ylmethyl)-5- (trifluoromethyl) isothiazole; 4- methylbenzene- sulfonic acid | 3- (bromo- methyl)-5- (trifluoro- methyl) isothiazole (CAS: 2253638- 60-5) | 292.2 [M + H]⁺ |
| B.387 | 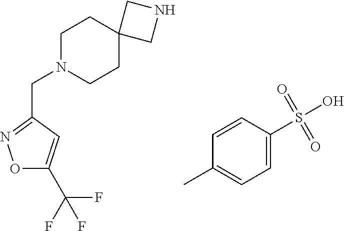 [SMILES: FC(F)(F)c1cc(CN2CCC3(CC2)CNC3)no1 ; Cc1ccc(S(=O)(=O)O)cc1] | 3-(2,7- diazaspiro[3.5] nonan- 7-ylmethyl)-5- (trifluoromethyl) isoxazole; 4- methylbenzene- sulfonic acid | 3- (bromo- methyl)-5- (trifluoro- methyl) isoxazole (CAS: 861135-68- 4) | 276.2 [M + H]⁺ |
| B.388 | 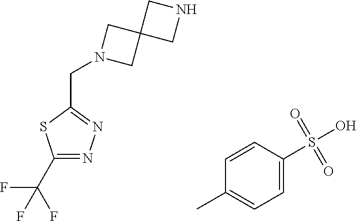 [SMILES: FC(F)(F)c1nnc(CN2CC3(CNC3)C2)s1 ; Cc1ccc(S(=O)(=O)O)cc1] | 2-(2,6- diazaspiro[3.3] heptan- 2-ylmethyl)-5- (trifluoromethyl)- 1,3,4-thiadiazole; 4- methylbenzene- sulfonic acid | 2- (chloro- methyl)-5- (trifluoro- methyl)- 1,3,4- thiadiazole (CAS: 1260672- 42-1) | 265.0 [M + H]⁺ |
| B.389 | 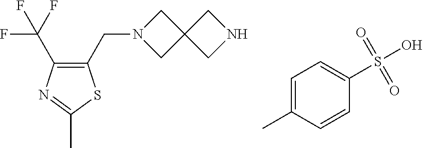 [SMILES: Cc1nc(C(F)(F)F)c(CN2CC3(CNC3)C2)s1 ; Cc1ccc(S(=O)(=O)O)cc1] | 5-(2,6- diazaspiro[3.3] heptan- 2-ylmethyl)-2- methyl-4- (trifluoromethyl) thiazole; 4- methylbenzene- sulfonic acid | 2-methyl-4- (trifluoro- methyl) thiazole-5- carbaldehyde (CAS: 1034566- 13-6) | 278.0 [M + H]⁺ |
| B.394 | 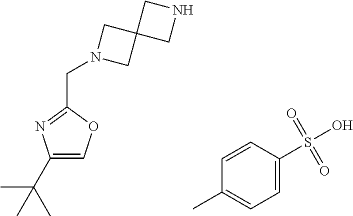 [SMILES: CC(C)(C)c1coc(CN2CC3(CNC3)C2)n1 ; Cc1ccc(S(=O)(=O)O)cc1] | 4-tert-butyl-2-(2,6- diazaspiro[3.3] heptan-2- ylmethyl)oxazole; 4- methylbenzene- sulfonic acid | 2- (bromo- methyl)- 4-tert- butyl- oxazole (CAS: 2126162- 62-5) | 236,0 [M + H]⁺ |
| B.396 | 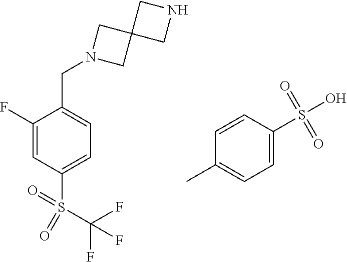 [SMILES: O=S(=O)(c1ccc(CN2CC3(CNC3)C2)c(F)c1)C(F)(F)F ; Cc1ccc(S(=O)(=O)O)cc1] | 2-[[2-fluoro-4- (trifluoromethyl- sulfonyl)phenyl] methyl]-2,6- diazaspiro[3.3] heptane; 4- methylbenzene- sulfonic acid | 2-fluoro-1- methyl-4- (trifluoro- methyl- sulfonyl) benzene (C.9) | 339.0 [M + H]⁺ |
| B.422 | 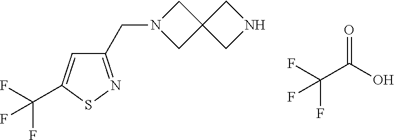 [SMILES: FC(F)(F)c1cc(CN2CC3(CNC3)C2)ns1 ; O=C(O)C(F)(F)F] | 3-(2,6- diazaspiro[3.3] heptan-2- ylmethyl)-5- (trifluoromethyl) isothiazole; 2,2,2- trifluoroacetic acid | 3- (bromo- methyl)-5- (trifluoro- methyl)-1,2- thiazole (CAS: 2253638- 60-5) | 264.0 [M − TFA + H]⁺ |
| B.424 | 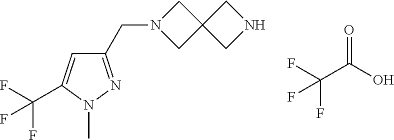 [SMILES: Cn1nc(CN2CC3(CNC3)C2)cc1C(F)(F)F ; O=C(O)C(F)(F)F] | 2-[[1-methyl-5- (trifluoromethyl) pyrazol-3-yl] methyl]-2,6- diazaspiro[3.3] heptane; 2,2,2- trifluoroacetic acid | 3- (chloro- methyl)-1- methyl-5- (trifluoro- methyl)-1H- pyrazole (CAS: 1260658- 92-1) | 261.2 [M − TFA + H]⁺ |
| B.428 | 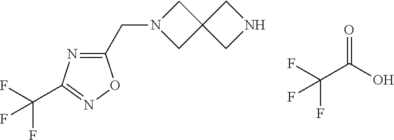 [SMILES: FC(F)(F)c1noc(CN2CC3(CNC3)C2)n1 ; O=C(O)C(F)(F)F] | 5-(2,6- diazaspiro[3.3] heptan-2- ylmethyl)-3- (trifluoromethyl)- 1,2,4-oxadiazole; 2,2,2-trifluoroacetic acid | 5- (chloro- methyl)-3- (trifluoro- methyl)- 1,2,4- oxadiazole (CAS: 100442-49- 7) | 249.0 [M − TFA + H]⁺ |
| B.433 | 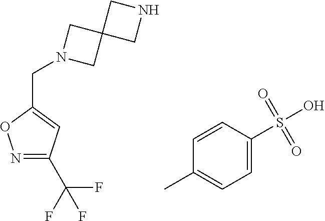 [SMILES: FC(F)(F)c1cc(CN2CC3(CNC3)C2)on1 ; Cc1ccc(S(=O)(=O)O)cc1] | 5-(2,6- diazaspiro[3.3] heptan-2- ylmethyl)-3- (trifluoromethyl) isoxazole; 4- methylbenzene- sulfonic acid | 5- (bromo- methyl)-3- (trifluoro- methyl) isoxazole (CAS: 1364677- 98-4) | 248,0 [M + H]⁺ |
| B.435 | 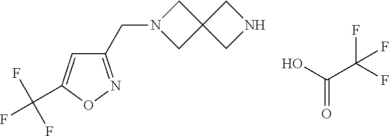 [SMILES: FC(F)(F)c1cc(CN2CC3(CNC3)C2)no1 ; O=C(O)C(F)(F)F] | 3-(2,6- diazaspiro[3.3] heptan-2- ylmethyl)-5- (trifluoromethyl) isoxazole; 2,2,2- trifluoroacetic acid | 3- (bromo- methyl)-5- (trifluoro- methyl) isoxazole (CAS: 861135-68- 4) | 248.2 [M − TFA + H]⁺ |
| B.437 | 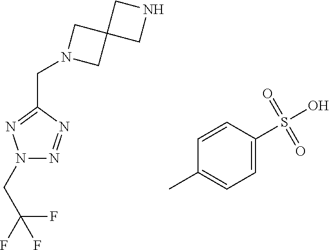 [SMILES: FC(F)(F)Cn1nnc(CN2CC3(CNC3)C2)n1 ; Cc1ccc(S(=O)(=O)O)cc1] | 2-[[2-(2,2,2- trifluoroethyl) tetrazol- 5-yl]methyl]-2,6- diazaspiro[3.3] heptane; 4- methylbenzene- sulfonic acid | 5- (chloro- methyl)- 2-(2,2,2- trifluoroethyl) tetrazole (CAS: 1803607- 11-5) | 263.0 [M − TsOH + H]⁺ |
| B.439 | 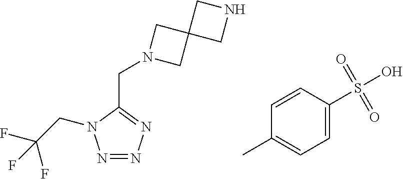 [SMILES: FC(F)(F)Cn1nnnc1CN1CC2(CNC2)C1 ; Cc1ccc(S(=O)(=O)O)cc1] | 2-[[1-(2,2,2- trifluoroethyl) tetrazol- 5-yl]methyl]-2,6- diazaspiro[3.3] heptane; 4- methylbenzene- sulfonic acid | 5- (chloro- methyl)- 1-(2,2,2- trifluoroethyl) tetrazole (CAS: 1341047- 82-2) | 263.0 [M − TsOH + H]⁺ |
| B.456 | 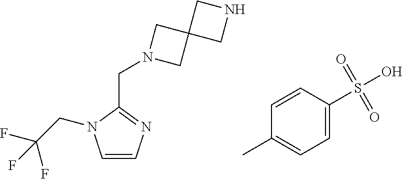 [SMILES: FC(F)(F)Cn1ccnc1CN1CC2(CNC2)C1 ; Cc1ccc(S(=O)(=O)O)cc1] | 2-[[1-(2,2,2- trifluoroethyl) imidazol- 2-yl]methyl]-2,6- diazaspiro[3.3] heptane; 4- methylbenzene- sulfonic acid | 2- (chloro- methyl)- 1-(2,2,2- trifluoroethyl) imidazole hydrochloride (CAS: 135206-93- 8) | 261.2 [M − TsOH + H]⁺ |
| B.470 | 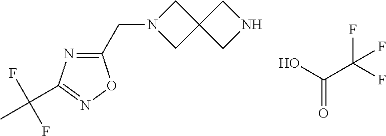 [SMILES: CC(F)(F)c1noc(CN2CC3(CNC3)C2)n1 ; O=C(O)C(F)(F)F] | 5-(2,6- diazaspiro[3.3] heptan- 2-ylmethyl)-3- (1,1-difluoroethyl)- 1,2,4-oxadiazole; 2,2,2-trifluoroacetic acid | 5- (chloro- methyl)- 3-(1,1- difluoro- ethyl)-1,2,4- oxadiazole (CAS: 1287685- 82-8) | 245.2 [M − TFA + H]⁺ |
| B.473 | 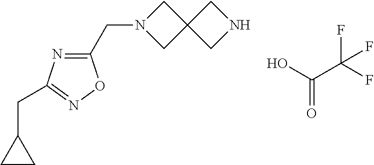 [SMILES: C1CC1Cc1noc(CN2CC3(CNC3)C2)n1 ; O=C(O)C(F)(F)F] | 3- (cyclopropyl- methyl)-5-(2,6- diazaspiro[3.3] heptan-2- ylmethyl)-1,2,4- oxadiazole; 2,2,2- trifluoroacetic acid | 5- (chloro- methyl)-3- (cyclopropyl- methyl)- 1,2,4- oxadiazole (CAS: 1506463- 66-6) | 235.2 [M − TFA + H]⁺ |
| B.525 | 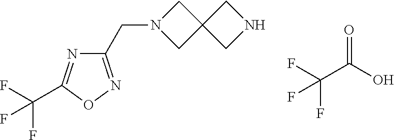 [SMILES: FC(F)(F)c1nc(CN2CC3(CNC3)C2)no1 ; O=C(O)C(F)(F)F] | 3-(2,6- diazaspiro[3.3] heptan-2- ylmethyl)-5- (trifluoromethyl)- 1,2,4-oxadiazole; 2,2,2-trifluoroacetic acid | 3- (chloro- methyl)-5- (trifluoro- methyl)- 1,2,4- oxadiazole (CAS: 890095-69- 9) | 249.0 [M − TFA + H]⁺ |
| B.444 | 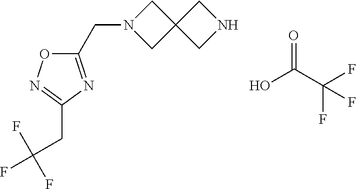 [SMILES: FC(F)(F)Cc1noc(CN2CC3(CNC3)C2)n1 ; O=C(O)C(F)(F)F] | 5-(2,6- diazaspiro[3.3] heptan-2- ylmethyl)-3- (2,2,2- trifluoroethyl)-1,2,4- oxadiazole; 2,2,2- trifluoroacetic acid | 5- (chloro- methyl)- 3-(2,2,2- trifluoro- ethyl)-1,2,4- oxadiazole (CAS: 1249397- 70-3) | 263.2 [M − TFA + H]⁺ |
| B.555 | 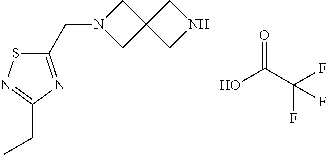 | 5-(2,6- diazaspiro[3.3] heptan-2- ylmethyl)-3- ethyl-1,2,4- thiadiazole; 2,2,2- trifluoroacetic acid | 5- (chloro- methyl)-3- ethyl-1,2,4- thiadiazole (CAS: 1312601- 20-9) | 225.0 [M − TFA + H]⁺ |

### Example B.110

### 2-[[4-fluoro-2-(trifluoromethyl)phenyl]methyl]-2,6-diazaspiro[3.3]heptane; trifluoroacetic Acid

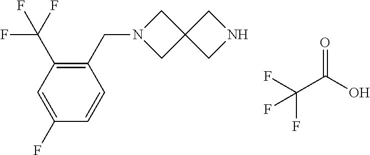 [SMILES: Fc1ccc(CN2CC3(CNC3)C2)c(C(F)(F)F)c1 ; O=C(O)C(F)(F)F]

To a solution of tert-butyl 6-(4-fluoro-2-(trifluoromethyl)benzyl)-2,6-diazaspiro[3.3]heptane-2-carboxylate (455 mg, 1.09 mmol) in dichloromethane (4 mL) was added TFA (843 μL, 10.9 μL) and the reaction mixture was stirred at RT for 18 h. Volatiles were removed in vacuo to yield 685 mg of the crude title compound (purity roughly 80%) which was used without further purification. MS (ESI): m/z=275.2 [M-TFA+H]⁺

### Step a) Tert-Butyl 6-(4 fluoro-2-(trifluoromethyl)benzoyl)-2,6-diazaspiro[3.3]heptane-2-carboxylate

To a solution of tert-butyl 2,6-diazaspiro[3.3]heptane-2-carboxylate (400 mg, 2.02 mmol) in CH₂Cl₂ (9 mL) cooled down to 0° C. was added DIPEA (652 mg, 881 μL, 5.04 mmol) and 4-fluoro-2-(trifluoromethyl)benzoyl chloride (503 mg, 2.22 mmol). The reaction mixture was stirred at 0° C. for 10 min and at RT for 18 h. The reaction mixture was diluted with dichloromethane and extracted with aq. Na₂CO₃ 1M solution, the organic phase was collected and the aqueous phase was back-extracted with dichloromethane. The combined organic phases were dried over sodium sulfate and evaporated down to dryness. The crude material was purified by flash chromatography, using an eluent mixture of heptane and ethyl acetate (5% to 80%) to give the title compound (569 mg). MS (ESI): m/z=389.3 [M+H]⁺

### Step b) Tert-Butyl 6-(4-fluoro-2-(trifluoromethyl)benzyl)-2,6-diazaspiro[3.3]heptane-2-carboxylate

To a solution of tert-butyl 6-(4-fluoro-2-(trifluoromethyl)benzoyl)-2,6-diazaspiro[3.3]heptane-2-carboxylate (565 mg, 1.45 mmol) in dry THE (5 mL) was slowly added borane tetrahydrofuran complex 1.0 M (3.64 mL, 3.64 mmol) and the reaction mixture was then refluxed for 20 h. The reaction was cooled down to 0° C. followed by addition of slow addition of methanol to quench excess borane after which it was stirred at 23° C. for 15 min followed by stirring at 55° C. for 18 h. Volatiles were removed in vacuo and the crude residue was directly purified by flash chromatography using an eluent mixture of dichloromethane and methanol (0% to 10%) to yield 417 mg of the title compound. MS (ESI): m/z=375.2 [M+H]⁺

### Example B.113

### 2-(2,6-diazaspiro[3.3]heptan-2 ylmethyl)-N-methyl-benzamide; 4-methylbenzenesulfonic Acid

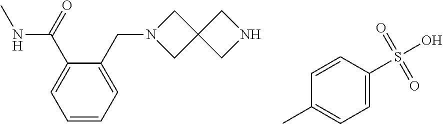 [SMILES: CNC(=O)c1ccccc1CN1CC2(CNC2)C1 ; Cc1ccc(S(=O)(=O)O)cc1]

A solution of tert-butyl 6-[[2-(methylcarbamoyl)phenyl]methyl]-2,6-diazaspiro[3.3]heptane-2-carboxylate (800 mg, 2.32 mmol) and p-toluenesulfonic acid monohydrate (1320 mg, 6.95 mmol) in EtOAc (60 mL) was heated at reflux for 2 h, then cooled to RT and stinted for another 16 h. The precipitate was collected by filtration, washed with ethyl acetate (20 mL) and dried under vacuum to provide the title compound (1069 mg, 1.81 mmol, 77% yield) as light yellow solid. MS (ESI): m/z=246.2 [M+H]⁺

### Step a) Tert-Butyl 6-[[2-(methylcarbamoyl)phenyl]methyl]-2,6-diazaspiro[3.3]heptane-2-carboxylate

tert-butyl 6-[(2-methoxycarbonylphenyl)methyl]-2,6-diazaspiro[3.3]heptane-2-carboxylate (Example B.112) (1000 mg, 2.89 mmol) was mixed with a solution of monomethylamine (43.3 mL, 866 mmol) (20% in MeOH) in a vial. Then the vial was sealed and heated at 70° C. for 24 h. The reaction mixture was then cooled to room temperature and concentrated in vacuo to give the title compound (970 mg, 92% yield) as light yellow viscous oil. The product was used in the next step without further purification. MS (ESI): m/z=346.2 [M+H]⁺

### Example B.114

### 2-[[2-[1-(methoxymethyl)-1,2,4-triazol-3-yl]phenyl]methyl]-2,6-diazaspiro[3.3]heptane; 4-methylbenzenesulfonic Acid

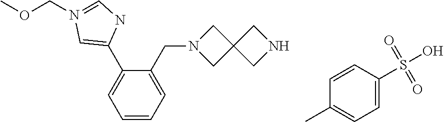

A solution of tert-butyl 6-[[2-[1-(methoxymethyl)-1,2,4-triazol-3-yl]phenyl]methyl]-2,6-diazaspiro[3.3]heptane-2-carboxylate (1050 mg, 2.63 mmol) and p-toluenesulfonic acid monohydrate (2000 mg, 10.5 mmol) in EtOAc (100 mL) was heated at reflux for 3 h, then cooled to RT and stirred for another 16 b. The precipitate was collected by filtration, washed with ethyl acetate (20 mL) and dried under vacuum to provide the title compound (1530 mg, 86% yield) as white solid. MS (ESI): m/z=300.2 [M+H]⁺

### Step a) 2-[1-(methoxymethyl)-1,2,4-triazol-3 yl]benzaldehyde

To a stirred suspension of 3-bromo-1-(methoxymethyl)-1,2,4-triazole (CAS: 1559064-16-2) (2.5 g, 13.0 mmol), cesium carbonate (1.06 g, 3.26 mmol), potassium carbonate (4.5 g, 32.6 mmol) and 2-formylphenylboronic acid (CAS: 40138-16-7) (2.54 g, 16.9 mmol) in 1,4-Dioxane (80 mL) and water (8 mL), flushed with Argon for 5 minutes, tetrakis(triphenylphosphine)palladium(0) (1054 mg, 0.910 mmol) was added. The mixture was stored at 100° C. for 18 h under an argon atmosphere (sealed tube). After cooling to RT, the reaction mixture was filtered and precipitate was washed with dioxane (50 mL). The filtrate was concentrated in vacuo and purified by flash chromatography to obtain 2 [1-(methoxymethyl)-1,2,4-triazol-3-yl]benzaldehyde (2.0 g. 67% yield) as a light yellow solid. MS (ESI): m/z=218.2 [M+H]⁺

### Step b) Tert-Butyl 6-[[2-[1-(methoxymethyl)-1,2,4-triazol-3 yl]phenyl]methyl]-2,6-diazaspiro[3.3]heptane-2-carboxylate

2-[1-(methoxymethyl)-1,2,4-triazol-3-yl]benzaldehyde (1000 mg, 4.6 mmol), tert-butyl 2,6-diazaspiro[3.3]heptane-2-carboxylate hydrochloride (1080 mg, 4.6 mmol), and triethylamine (1.92 mL, 13.8 mmol) were mixed in DCM (50 mL) and stirred for 20 min at RT. Then sodium triacetoxyborohydride (1.95 g, 9.21 mmol) was added in one portion, and the reaction mixture was stirred for 18 h at RT. Then reaction mixture was diluted with DCM (50 mL), and 5% NaHCO₃ (80 mL) was added. The organic phase was separated, and the aqueous layer was extracted with DCM (50 mL). The organic layers were combined, washed with brine (50 mL), dried over Na₂SO₄ and concentrated in vacuo.

The cude product was purified by flash chromatography to obtain the title compound (1050 mg, 54% yield) as a yellow oil. MS (ESI): m/z=400.2 [M+H]⁺

In analogy to Example B.114, the following building blocks were generated using the relevant commercial building blocks in Step a).

| | | | | MS, |
|---|---|---|---|---|
| | | | Building | ESI: |
| Ex. | Structure | Systematic Name | Blocks | m/z |
| B.115 | 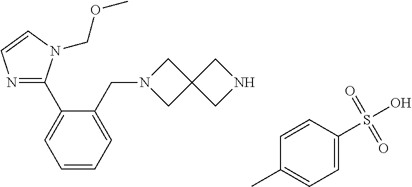 [SMILES: COCn1ccnc1-c1ccccc1CN1CC2(CNC2)C1 ; Cc1ccc(S(=O)(=O)O)cc1] | 2-[[2-[1- (methoxymethyl) imidazol-2- yl]phenyl]methyl]- 2,6- diazaspiro[3.3] heptane; 4- methylbenzene- sulfonic acid | 2-bromo-1- (methoxy- methyl) imidazole (CAS: 1001755- 89-0) | 299.4 [M + H]⁺ |

### Example B.139

### N-cyclopropyl-N-[[2-fluoro-4-(trifluoromethyl)phenyl]methyl]azetidin-3-amine; 4-methylbenzenesulfonic Acid

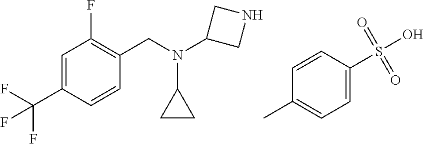 [SMILES: Fc1cc(C(F)(F)F)ccc1CN(C1CC1)C1CNC1 ; Cc1ccc(S(=O)(=O)O)cc1]

3-[cyclopropyl-[2-fluoro-4-(trifluoromethyl)benzyl]amino]azetidine-1-carboxylic acid tert-butyl ester (561 mg, 1.44 mmol) was dissolved in ethyl acetate (30 mL) and p-toluenesulfonic acid monohydrate (563 mg, 2.96 mmol) was added at room temperature. The solution was stirred over night under reflux. The mixture was extracted with EtOAc (20 mL), and washed with Na₂CO₃ (20 mL/10% aq. solution) and brine (20 mL). The organic layer was dried over MgSO₄ and concentrated in vacuo to obtain a yellow oil. The crude material was purified by flash chromatography (0% Heptane to 100% (TBME: MeOH 9:1) to 100% MeOH. The fractions were collected and dried to yield the title compound as light yellow oil (164 mg, 34.7%) MS (ESI): m/z=289.2 [M+H]⁺

### Step a) 3-[cyclopropyl-[2-[fluoro-4-(trifluoromethyl)benzyl]amino]azetidine-1-carboxylic Acid Tert-Butyl Ester

A solution of 2-fluoro-4-(trifluoromethyl)benzaldehyde (388 mg, 2.02 mmol) and 3-(cyclopropylamino)azetidine-1-carboxylic acid tert-butyl ester (CAS: 1342433-96-8) (500 mg, 2.36 mmol) in dichloromethane (10 mL) and acetic acid (297 mg, 283 μL, 4.94 mmol) was stirred for 10 min at room temperature, then sodium triacetoxyborohydride (1.21 g, 5.73 mmol) was added. The mixture was stirred at RT for 2 h. A further addition of sodium triacetoxyborohydride (424 mg, 2.0 mmol) was made, and the reaction was stirred over night. The milky white mixture was dissolved in DCM (20 mL), poured into NaHCO₃ sat. aq. solution (20 mL) and stirred for 2.5 h. The mixture was extracted with dichloromethane (30 mL), and the combined organics washed with water (30 mL) and NaCl sat. aq. (30 mL). The organic layer was dried over Na₂SO₄ and concentrated in vacuo to obtain a colorless oil. The residue was purified by column chromatography. The fractions were collected and dried to yield the title compound as colorless oil (561 mg, 60%). MS (ESI): m/z=333.2 [M-tBu+H]⁺

### Example B.140

### N-(cyclopropylmethyl)-N-[[2-fluoro-4-(trifluoromethyl)phenyl]methyl]azetidin-3-amine; 4-methylbenzenesulfonic Acid

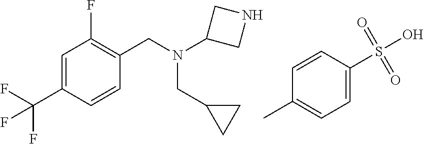

3-[cyclopropylmethyl-[2-fluoro-4-(trifluoromethyl)benzyl]amino]azetidine-1-carboxylic acid tert-butyl ester (343 mg, 0.852 mmol) was dissolved in ethyl acetate (8 mL) and p-toluenesulfonic acid monohydrate (357 mg, 1.88 mmol) was added at room temperature and the mixture was stirred over night under reflux. The cooled reaction mixture was extracted with ethyl acetate (2×15 ml) and washed with Na₂CO₃ (15 mL/10% aq. solution). The organic layer was dried over MgSO₄ and concentrated in vacuo to obtain the title compound (214 mg, 59%) as a yellow oil. MS (ESI): m/z=303.2 [M+H]⁺

### Step a) 3-[[2-fluoro-4-(trifluoromethyl)benzyl]amino]azetidine-1-carboxylic Acid Tert-Butyl Ester

To a solution of 2-fluoro-4-(trifluoromethyl)benzaldehyde (2.8 g, 14.6 mmol) and 3-aminoazetidine-1-carboxylic acid tert-butyl ester (2.76 g, 16.0 mmol) in dichloromethane (30 mL) was added sodium triacetoxyborohydride (3.4 g, 16.0 mmol) and acetic acid (1.75 g, 1.67 mL, 29.2 mmol). The white suspension was stirred at RT overnight. The reaction mixture was poured into DCM (100 mL) and washed with NaHCO₃ sat. aq. solution (100 mL) and water (100 mL), and brine (100 mL). The organic layer was dried over Na₂SO₄ and concentrated in vacuo as an colorless oil. The crude material was purified by flash chromatography to afford the title compound (2710 mg, 54%) as light yellow oil. MS (ESI): m/z=293.2 [M-Boc+H]⁺

### Step b) 3-[cyclopropylmethyl-[2 fluoro-4-(trifluoromethyl)benzyl]amino]azetidine-1-carboxylic Acid Tert-Butyl Ester

3-[[2-fluoro-4-(trifluoromethyl)benzyl]amino]azetidine-1-carboxylic acid tert-butyl ester (750 mg, 2.15 mmol) was dissolved in N,N-dimethylformamide (3 mL). Cesium carbonate (1.75 g, 5.38 mmol) was added at RT and the mixture was stirred for 1 h. Then bromomethylcyclopropane (436 mg, 314 μL, 3.23 mmol) was added slowly and the white suspension was stirred at 40° C. for 3 h. The white suspension was extracted two times with EtOAc. The organic layers were washed with water (2×50 mL), dried over Na₂SO₄ and concentrated to dryness. The crude material was purified by flash chromatography (eluting with heptane:TBME 95% to 60%) to afford the title compound (343 mg, 39.6%) as a light yellow oil. MS (ESI): m/z=403.2 [M+H]⁺

### Example B.145

### 1-(azetidin-3-yl)-4-(2,2,2-trifluoroethoxy)pyrazole; 4-methylbenzenesulfonic Acid

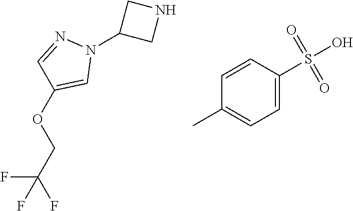

To a solution of tert-butyl 3-[4-(2,2,2-trifluoroethoxy)pyrazol-1-yl]azetidine-1-carboxylate (880 mg, 2.74 mmol) in EtOAc (25 mL) was added p-toluenesulfonic acid (566 mg, 3.29 mmol), the mixture was stirred at 80° C. for 12 h. The mixture was cooled to 20° C. and stirred for another 2 h, filtered and the filter cake was washed with EtOAc (20 mL). The filter cake was collected and concentrated to give the title compound (855 mg, 79% yield) as an off-white solid. MS (ESI): m/z=222.1 [M-TsOH+H]⁺

### Step a) Tert-Butyl 3-[4-(4,4,5,5-tetramethyl-1,3,2-dioxaborolan-2 yl)pyrazol-1-yl]azetidine-1-carboxylate

To a solution of 4-pyrazoleboronic acid pinacol ester (CAS: 269410-08-4) (3.0 g, 15.5 mmol) and 1-BOC-3-iodoazetidine (CAS: 254454-54-1) (4.38 g, 15.5 mmol) in DMF (75 mL) was added cesium carbonate (10.1 g, 30.9 mmol), the mixture was stirred at 90° C. under N₂ for 16 h. The mixture was diluted by water (300 mL) and extracted with EtOAc (100 mL three times), washed by brine (100 mL) and dried by Na₂SO₄. The combined organic phase was concentrated and purified by reversed flash (0.05% v/v FA condition) to give the title compound (1.9 g, 35% yield) as a yellow oil. MS (ESI): m/z=294.4 [M-C₄H₈+H]⁺

### Step b) Tert-Butyl 3-(4-hydroxypyrazol-1 yl)azetidine-1-carboxylate

To a cold (0° C., ice bath) solution of tert-butyl 3-[4-(4,4,5,5-tetramethyl-1,3,2-dioxaborolan-2-yl)pyrazol-1-yl]azetidine-1-carboxylate (1900 mg, 5.44 mmol) in THE (40 mL) was slowly added another solution of sodium hydroxide (435 mg, 10.9 mmol) in water (4 mL), followed by hydrogen peroxide (1234 mg, 10.9 mmol). The reaction mixture was stirred at 0-20° C. for 3 h. LCMS showed the reaction was complete. The mixture was carefully neutralized to pH=5 with 1 N HCl and diluted with EtOAc (50 mL). The aqueous layer was extracted with EtOAc (50 mL three times). The combined organic layers were washed with Na₂SO₃ solution and brine, dried over Na₂SO₄ and concentrated under reduced pressure to afford the crude title compound (1.3 g, 99.9% yield) as a light yellow oil. MS (ESI): m/z=184.5 [M-C₄H₈+H]⁺

### Step c) Tt-Butyl 3-[4-(2,2,2-trifluoroethoxy)pyrazol-1 yl]azetidine-1-carboxylate

To a solution of tert-butyl 3-(4-hydroxypyrazol-1-yl)azetidine-1-carboxylate (1.3 g, 5.43 mmol) in DMF (30 mL) was added NaH (0.26 g, 6.52 mmol) at 0° C., stirred for 30 min. Then 2,2,2-trifluoroethyl trifluoromethanesulfonate (CAS: 6226-25-1) (1.28 mL, 8.15 mmol) was added dropwise at 0° C., the mixture was stirred at 20° C. for 2 h. The mixture was diluted by water (300 mL) and extracted with EtOAc (100 mL three times). The combined organic phase was washed by brine (100 mL), dried by Na₂SO₄, concentrated and purified by reversed flash (0.05% v/v FA condition) to give the title compound (982 mg, 52% yield) as yellow oil. MS (ESI): m/z=266.0 [M-C₄H₈+H]⁺

### Example B.149

### N-(1-methylcyclopropyl)-2,6-diazaspiro[3.3]heptane-2-sulfonamide; 2,2,2-trifluoroacetic Acid

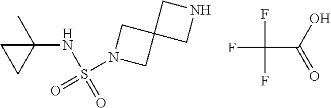

To a solution of 2-[(1-methylcyclopropyl)sulfamoyl]-2,6-diazaspiro[3.3]heptane-6-carboxylic acid tert-butyl ester (448 mg, 1.35 mmol) in dichloromethane (5 mL) was added TFA (1.54 g, 1.04 mL, 13.52 mmol, 10 eq) and the reaction mixture was stirred at room temperature for 18 h. Volatiles were removed in vacuo to yield the crude title compound (736 mg), roughly 63% purity with excess TFA as major contaminant, which was used directly without further purification. MS (ESI): m/z=232.2 [M-TFA+H]⁺

### Step a) 2-(2-methylimidazol-1 yl)sulfonyl-2,6-diazaspiro[3.3]heptane-6-carboxylic Acid Tert-Butyl Ester

To a solution of 2-methyl-1-(2-methylimidazol-1-yl)sulfonyl-imidazole (1.5 g, 6.63 mmol) in dichloromethane (27 mL) under an inert atmosphere cooled down to 0° C. was slowly added methyl trifluoromethanesulfonate (1.09 g, 730 μL, 6.63 mmol). Upon completion of reagent addition a white precipitate began to form and the reaction mixture was allowed to stir at 0° C. and slowly warm up to room temperature overnight. Volatiles were removed in vacuo to give 2.60 g of the crude intermediate as a white solid which was used without further purification. The crude solid was dissolved in acetonitrile, extra dry (27 mL) followed by addition of 2,6-diazaspiro[3.3]heptane-2-carboxylic acid tert-butyl ester (1.31 g, 6.63 mmol) after which the reaction mixture was stirred at 80° C. for 64 h. The reaction mixture was diluted with ethyl acetate, poured into a separating funnel and extracted with aq. sol. Na₂CO₃ 1 M. The organic phase was collected and the aqueous phase was back-extracted with ethyl acetate. The combined organic phases were dried over sodium sulfate and evaporated down to dryness. The crude material was submitted for SFC purification to yield 1372 mg of the title compound. MS (ESI): m/z=343.2 [M-TFA+H]⁺

### Step b) 2-[(1-methylcyclopropyl)sulfamoyl]-2,6-diazaspiro[3.3]heptane-6-carboxylic Acid Tert-Butyl Ester

To a solution of 2-(2-methylimidazol-1-yl)sulfonyl-2,6-diazaspiro[3.3]heptane-6-carboxylic acid tert-butyl ester (778 mg, 2.27 mmol) in dichloromethane (10 mL) cooled down to 0° C. was added methyl trifluoromethanesulfonate (392 mg, 263 μL, 2.39 mmol) and the reaction mixture was stirred at 0° C. for 3 h. Volatiles were removed in vacuo and the crude white solid was re-dissolved in acetonitrile, extra dry (10 mL) followed by addition of (1-methylcyclopropyl)amine (242 mg, 3.41 mmol) after which the reaction mixture was stirred at 70° C. for 18 h. Volatiles were removed in vacuo. The crude residue was dissolved in ethyl acetate, transferred into a separating funnel and extracted with sat. aq. solution Na₂CO₃. The organic phase was collected and the aqueous phase was back-extracted with ethyl acetate. The combined organic phases were dried over sodium sulfate and evaporated down to dryness. Purification by FC (SiO₂; DCM/MeOH) gave the title compound (448 mg). MS (ESI): m/z=330.3 [M+H]⁺

In analogy to Example B.149, the following building blocks were generated using the relevant commercial building blocks in Step b).

| | | | | MS, |
|---|---|---|---|---|
| | | | Building | ESI: |
| Ex. | Structure | Systematic Name | Blocks | m/z |
| B.150 | 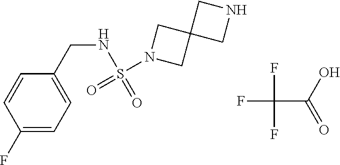 [SMILES: O=S(=O)(NCc1ccc(F)cc1)N1CC2(CNC2)C1 ; O=C(O)C(F)(F)F] | N-[(4- fluorophenyl) methyl]-2,6- diazaspiro[3.3] heptane- 2-sulfonamide; trifluoroacetic acid | (4- fluorobenzyl) amine | 286.1 [M − TFA + H]⁺ |
| B.151 | 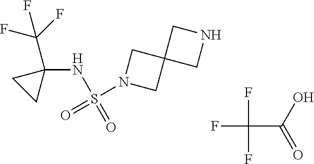 [SMILES: O=S(=O)(NC1(C(F)(F)F)CC1)N1CC2(CNC2)C1 ; O=C(O)C(F)(F)F] | N-[1- (trifluoromethyl) cyclopropyl]-2,6- diazaspiro[3.3] heptane-2- sulfonamide; trifluoroacetic acid | [1- (trifluoro- methyl) cyclopropyl] amine | 286.1 [M − TFA + H]⁺ |

### Example B.152

### N-[[1-(trifluoromethyl)cyclopropyl]methyl]-2,6-diazaspiro[3.3]heptane-2-sulfonamide; 4-methylbenzenesulfonic Acid

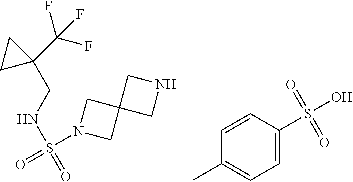 [SMILES: O=S(=O)(NCC1(C(F)(F)F)CC1)N1CC2(CNC2)C1 ; Cc1ccc(S(=O)(=O)O)cc1]

A solution of tert-butyl 2-[[1-(trifluoromethyl)cyclopropyl]methylsulfamoyl]-2,6-diazaspiro[3.3]heptane-6-carboxylate (450 mg, 1.13 mmol) and p-toluenesulfonic acid monohydrate (429 mg, 2.25 mmol) in EtOAc (15 mL) was heated at reflux for 2 h, then cooled to RT and stirred for another 16 h. The precipitate was collected by filtration, washed with EtOAc (5 mL) and dried under vacuum to provide the title compound (298 mg, 53% yield). MS (ESI): m/z=300.2 [M-TsOH+H]⁺

### Step a) Tert-Butyl 2-chlorosulfonyl-2,6-diazaspiro[3.3]heptane-6-carboxylate

To a stirred solution of sulfinyl chloride (0.63 g, 4.69 mmol) in DCM (15 mL) at 0° C. was added a mixture of triethylamine (1.19 mL, 8.52 mmol) and tert-butyl 2,6-diazaspiro[3.3]heptane-2-carboxylate hydrochloride (1.0 g, 4.26 mmol) (as a solution in 15 mL of DCM) at such a rate as to keep the temperature below 20° C. The reaction mixture was stirred at room temperature for 18 h, then evaporated to dryness. The crude sulfamoyl chloride (30% purity) was used directly in next step without further purification.

### Step b) Tert-Butyl 2-[[1-(trifluoromethyl)cyclopropyl]methylsulfamoyl]-2,6-diazaspiro[3.3]heptane-6-carboxylate

To a stirred mixture of tert-butyl 2-chlorosulfonyl-2,6-diazaspiro[3.3]heptane-6-carboxylate (310 mg, 1.04 mmol) and [1-(trifluoromethyl)cyclopropyl]methanamine; hydrochloride (238 mg, 1.36 mmol) in ACN (10 mL), N,N-diisopropylethylamine (0.55 mL, 3.13 mmol) was added. Then the tube was was sealed and stirred at 40° C. for 18 h. Then the reaction mixture was concentrated to dryness and the residue was taken up in DCM (20 mL) and the organics washed with water (2×5 mL) and saturated brine solution (5 mL). The organic layer was dried (Na₂SO₄) before being concentrated to dryness in vacuo. The (290 mg, 0.730 mmol, 66% yield) was used to the next step without further purification. MS (ESI): m/z=398.2 [M−H]⁻

### Example B.153

### 2-[5-(trifluoromethyl)pyrazin-2-yl]-2,6-diazaspiro[3.3]heptane; 4-methylbenzenesulfonic Acid

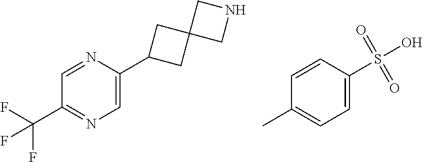 [SMILES: FC(F)(F)c1cnc(C2CC3(CNC3)C2)cn1 ; Cc1ccc(S(=O)(=O)O)cc1]

In a flask was added 6-[5-(trifluoromethyl)pyrazin-2-yl]-2,6-diazaspiro[3.3]heptane-2-carboxylic acid tert-butyl ester (312 mg, 0.906 mmol), p-toluenesulfonic acid monohydrate (361.96 mg, 1.9 mmol) in ethyl acetate (7.64 mL) the mixture was stirred at reflux overnight. The crude residue was washed with EtOAc and diethylether to afford the title compound as an off-white solid with an assumed purity of 95%, which was used without further purification. MS (ESI): m/z=245.1 [M-TsOH+H]⁺

### Step a) 6-[5-(trifluoromethyl)pyrazin-2-yl]-2,6-diazaspiro[3.3]heptane-2-carboxylic Acid Tert-Butyl Ester

In a flask was added 2-bromo-5-(trifluoromethyl)pyrazine (280 mg, 1.23 mmol), 2-Boc-2,6-diazaspiro[3.3]heptane (269 mg, 1.36 mmol) and cesium carbonate (804 mg, 2.47 mmol) in 1,4-dioxane (6. mL). The suspension was bubbled with N₂ for 5 min and chloro(2-dicyclohexylphosphino-2′,6′-diisopropoxy-1,1′-biphenyl)[2-(2′-amino-1,1′-biphenyl)]palladium(II) (47.9 mg, 0.062 mmol) was added. The mixture was heated at 110° C. for 2 h. The mixture was diluted in EtOAc and filtered through celite, the filtrate was concentrated. Purification by FC(SiO₂; heptane/EtOAc) gave the title compound as a yellow solid which was directly used in the next step. MS (ESI): m/z=345.1 [M+H]⁺

In analogy to Example B. 153, the following building blocks were generated using the relevant commercial building blocks.

| | | | | MS, |
|---|---|---|---|---|
| | | | Building | ESI: |
| Ex. | Structure | Systematic Name | Blocks | m/z |
| B.372 | 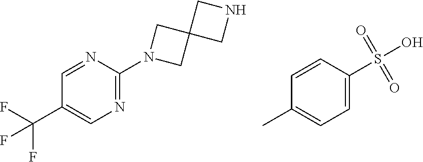 [SMILES: FC(F)(F)c1cnc(N2CC3(CNC3)C2)nc1 ; Cc1ccc(S(=O)(=O)O)cc1] | 2-[5- (trifluoromethyl) pyrimidin-2-yl]-2,6- diazaspiro[3.3] heptane; 4- methylbenzene- sulfonic acid | 2-bromo-5- (trifluoro- methyl) pyrimidine | 245.1 [M − TsOH + H]⁺ |

### Example B.154

### 1-(azetidin-3-ylmethyl)-4,4-difluoro-piperidine; 4-methylbenzenesulfonic Acid

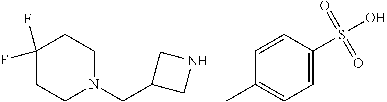

To a solution of 3-[(4,4-difluoropiperidino)methyl]azetidine-1-carboxylic acid tert-butyl ester (780 mg, 2.55 mmol) in isopropyl acetate (12 mL) was added p-toluenesulfonic acid monohydrate (971 mg, 5.1 mmol). The mixture was stirred at 80° C. for 3 h to give a white suspension. The reaction mixture was filtered through sintered glass, washed twice with Et₂O, dried in vacuo to afford the title compound (1.32 g, 92%) as white solid. MS (ESI): m/z=191.2 [M-TsOH+H]⁺

### Step a) 3-[(4,4-difluoropiperidino)methyl]azetidine-1-carboxylic Acid Tert-Butyl Ester

To a solution of 3-formylazetidine-1-carboxylic acid tert-butyl ester (0.630 g, 3.4 mmol), 4,4-difluoropiperidine; hydrochloride (536 mg, 3.4 mmol) and Et₃N (344 mg, 474 μL, 3.4 mmol) in dichloromethane (8 mL) was added sodium triacetoxy borohydride (865 mg, 4.08 mmol). The mixture was stirred at RT for 3 h. The reaction mixture was poured into EtOAc/THF 2:1 and washed with sat. aq. NaHCO₃/NaCl solution. The organic layer was dried over Na₂SO₄ and evaporated. Purification by FC(SiO₂; heptane/EtOAc) gave the title compound (784 mg, 75%) as a white solid. MS (ESI): m/z=235.2 [M-C₄H₈+H]⁺

### Example B.157

### N-(2-azaspiro[3.3]heptan-6-yl)-1-(trifluoromethyl)cyclopropanecarboxamide; 4-methylbenzenesulfonic Acid

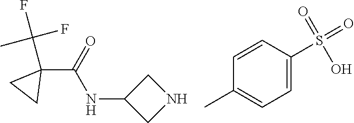

A solution of tert-butyl 6-[[1-(trifluoromethyl)cyclopropanecarbonyl]amino]-2-azaspiro[3.3]heptane-2-carboxylate (729 mg, 1.73 mmol, 67% yield) and p-toluenesulfonic acid monohydrate (0.98 g, 5.17 mmol) in EtOAc (50 mL) was heated at reflux for 2 h, then cooled to RT and stirred for another 16 h. The precipitate was collected by filtration, washed with ethyl acetate (20 mL) and dried under vacuum to provide N-(2-azaspiro[3.3]heptan-6-yl)-1-(trifluoromethyl)cyclopropanecarboxamide; 4-methylbenzenesulfonic acid (729 mg, 67% yield) as a white solid. MS (ESI): m/z=249.1 [M-TsOH+H]+

### Step a) Tert-Butyl 6-[[(1-(trifluoromethyl)cyclopropanecarbonyl]amino]-2-azaspiro[3.3]heptane-2-carboxylate

To a stirred solution of 1-(trifluoromethyl)cyclopropane-1-carboxylic acid (0.5 g, 3.22 mmol), tert-butyl 6-amino-2-azaspiro[3.3]heptane-2-carboxylate; hydrochloride (0.8 g, 3.22 mmol) and O-(7-azabenzotriazol-1-yl)-N,N,N′,N′-tetramethyluronium hexafluorophosphate (1.47 g, 3.86 mmol) (HATU) in DMF (8 mL), N,N-diisopropylethylamine (2.24 mL, 12.9 mmol) was added in one portion at RT. The resulting mixture was stirred overnight (18 h) at RT. Then poured onto water (50 mL), and resulting precipitate was filtered, washed with water and dried to give the title compound (0.900 g, 2.58 mmol, 76% yield) as a yellow solid. MS (ESI): m/z=347.2 [M+H]⁺

### Example B.161

### 3-(4-(1-(trifluoromethyl)cyclopropyl)phenyl)azetidine 4-methylbenzenesulfonate

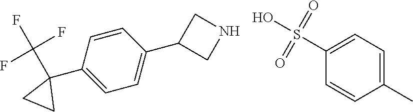

To a solution of tert-butyl 3-[4-[1-(trifluoromethyl)cyclopropyl]phenyl]azetidine-1-carboxylate (7.00 g, 20.5 mmol) in ethyl acetate (70 mL) was added p-toluenesulfonic acid (4.24 g, 24.6 mmol). The mixture was stinted at 80° C. for 3 h, cooled to room temperature, filtered and the filter cake was collected to give 4-methylbenzenesulfonic acid; 3-[4-[1-(trifluoromethyl)cyclopropyl]phenyl]azetidine (7600 mg, 89.6% yield) as a white solid. MS (ESI): m/z=242.4 [M-TsOH+H]⁺

### Step a) Tert-Butyl 3-[4-[1-(trifluoromethyl)cyclopropyl]phenyl]azetidine-1-carboxylate

To a 500 mL vial equipped with a stir bar was added tert-butyl 3-bromoazetidine-1-carboxylate (CAS: tert-butyl 3-bromoazetidine-1-carboxylate) (8017 mg, 34.0 mmol), 1-bromo-4-(1-trifluoromethyl-cyclopropyl)-benzene (CAS: 1-bromo-4-(1-trifluoromethyl-cyclopropyl)-benzene) (9000 mg, 34.0 mmol), Ir[dF(CF3)ppy]₂(dtbbpy)PF₆ (381 mg, 0.340 mmol), NiCl₂glyme (37.3 mg, 0.170 mmol), 4-tert-butyl-2-(4-tert-butyl-2-pyridyl)pyridine (54.7 mg, 0.200 mmol), bis(trimethylsilyl)silyl-trimethyl-silane (8443 mg, 34.0 mmol) and Na₂CO₃ (7197 mg, 67.9 mmol) in DME (225 mL). The vial was sealed and placed under nitrogen. The reaction was stirred and irradiated with a 34 W blue LED lamp (7 cm away), with cooling fan to keep the reaction temperature at 25° C. for 20 h. LCMS showed the reaction was complete, the reaction was filtered and the filtrate was concentrated, the residue was purified by reverse phase flash chromatography (FA) and concentrated to give tert-butyl 3-[4-[1-(trifluoromethyl)cyclopropyl]phenyl]azetidine-1-carboxylate (7700 mg, 66.4% yield) as a light yellow solid. MS: MS (ESI): m/z=286.0 [M-C₄H₈+H]⁺

In analogy to Example B.161, the following building blocks were generated using the relevant commercial building blocks in Step a).

| | | | | MS, |
|---|---|---|---|---|
| | | | Building | ESI: |
| Ex. | Structure | Systematic Name | Blocks | m/z |
| B.162 | 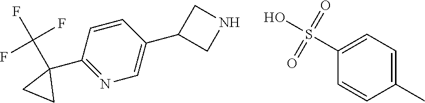 [SMILES: FC(F)(F)C1(c2ccc(C3CNC3)cn2)CC1 ; Cc1ccc(S(=O)(=O)O)cc1] | 5-(azetidin-3-yl)-2- [1- (trifluoromethyl) cyclopropyl] pyridine;4- methylbenzene- sulfonic acid | C.2 (5- bromo-2-[1- (trifluoro- methyl) cyclopropyl] pyridine) | 243.1 [M − TsOH + H]⁺ |
| B.163 | 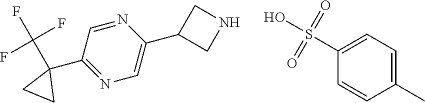 [SMILES: FC(F)(F)C1(c2cnc(C3CNC3)cn2)CC1 ; Cc1ccc(S(=O)(=O)O)cc1] | 2-(azetidin-3-yl)-5- [1- (trifluoromethyl) cyclopropyl] pyrazine; 4- methylbenzene- sulfonic acid | C.3 (2- chloro-5-[1- (trifluoro- methyl) cyclopropyl] pyrazine) | 244.5 [M − TsOH + H]⁺ |
| B.164 | 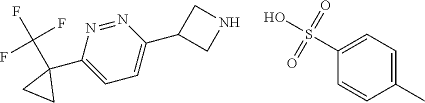 [SMILES: FC(F)(F)C1(c2ccc(C3CNC3)nn2)CC1 ; Cc1ccc(S(=O)(=O)O)cc1] | 3-(azetidin-3-yl)-6- [1- (trifluoromethyl) cyclopropyl] pyridazine; 4- methylbenzene- sulfonic acid | C.4 (3- bromo-6-[1- (trifluoro- methyl) cyclopropyl] pyridazine) | 244.4 [M − TsOH + H]⁺ |
| B.165 | 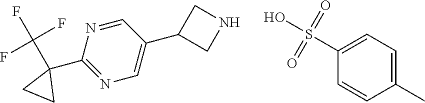 [SMILES: FC(F)(F)C1(c2ncc(C3CNC3)cn2)CC1 ; Cc1ccc(S(=O)(=O)O)cc1] | 5-(azetidin-3-yl)-2- [1- (trifluoromethyl) cyclopropyl] pyrimidine; 4- methylbenzene- sulfonic acid | C.5 (5- bromo-2-[1- (trifluoro- methyl) cyclopropyl] pyrimidine) | 244.5 [M − TsOH + H]⁺ |
| B.170 | 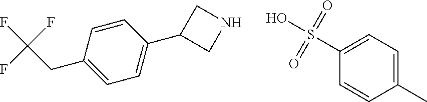 | 3-[4-(2,2,2- trifluoroethyl) phenyl]azetidine; 4- methylbenzene- sulfonic acid | 1-bromo-4- (2,2,2- trifluoro- ethyl) benzene (CAS: 155820-88- 5) | 216.2 [M − TsOH + H]⁺ |
| B.173 | 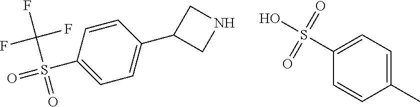 [SMILES: O=S(=O)(c1ccc(C2CNC2)cc1)C(F)(F)F ; Cc1ccc(S(=O)(=O)O)cc1] | 3-[4- (trifluoromethyl- sulfonyl) phenyl]azetidine; 4- methylbenzene- sulfonic acid | 1-bromo-4- [(trifluoro- methyl) sulfonyl] benzene | 266.1 [M − TsOH + H]⁺ |
| B.279 | 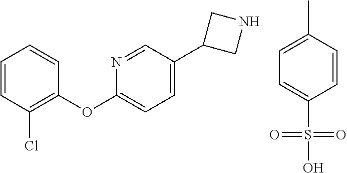 | 5-(azetidin-3-yl)-2- (2- chlorophenoxy) pyridine; 4- methylbenzene- sulfonic acid | 5-Bromo-2- (2- chloro- phenoxy) pyridine (CAS RN: 1240670- 82-9) | 261.1 [M + H]⁺ |
| B.282 | 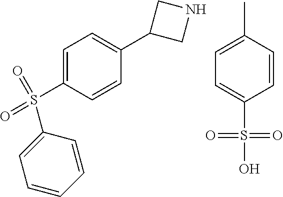 | 3-[4- (benzenesulfonyl) phenyl]azetidine; 4- methylbenzene- sulfonic acid | 1-Bromo-4- (phenyl- sulfonyl) benzene (CAS RN: 23038-36-0) | 274.2 [M + H]⁺ |
| B.283 | 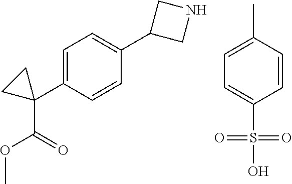 | methyl 1-[4- (azetidin-3-yl) phenyl]cyclopropane- carboxylate; 4- methylbenzene- sulfonic acid | Methyl 1- (4- bromo- phenyl) cyclopro- panecar- boxylate (CAS RN: 638220-35- 6 | 232.5 [M − Tos + H]⁺ |
| B.284 | 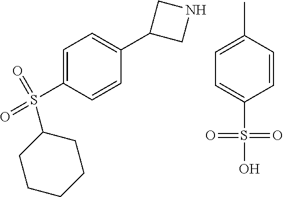 | 3-(4- cyclohexylsulfonyl- phenyl)azetidine; 4- methylbenzene- sulfonic acid | 1-Bromo-4- (cyclo- hexane- sulfonyl) benzene (CAS RN: 861113-45- 3) | 280,2 [M + H]⁺ |
| B.286 | 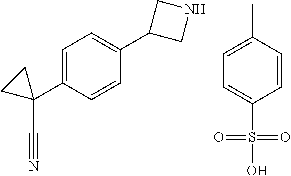 | 1-[4-(azetidin-3-yl) phenyl]cyclopropane- carbonitrile; 4- methylbenzene- sulfonic acid | 1-(4- Bromo- phenyl) cyclopro- panecarboni- trile (CAS RN: 124276-67- 1) | 199.5 [M − Tos + H]⁺ |
| B.290 | 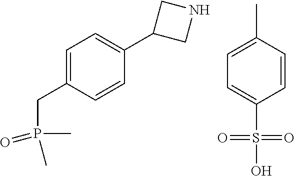 | 3-[4- (dimethylphosphoryl- methyl)phenyl] azetidine; 4- methylbenzene- sulfonic acid | 1-bromo-4- [(dimethyl- phosphoryl) methyl] benzene (CAS RN: 119199-90- 5) | 224.3 [M + H]⁺ |
| B.203 | 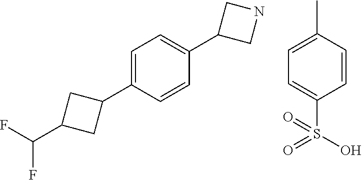 | 3-[4-[3- (Difluoromethyl) cyclobutyl] phenyl]azetidine; 4- methylbenzene- sulfonic acid | tert-butyl 3- (4- bromo- phenyl) azetidine-1- carboxylate (CAS RN 1203681- 52-0) and 1- bromo-3- (difluoro- | 238.2 [M + H]⁺ |
| | | | methyl) | |
| | | | cyclobutane | |
| | | | (CAS RN: | |
| | | | 2241140- | |
| | | | 79-2) | |
| B.296 | 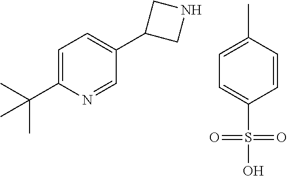 | 5-(azetidin-3-yl)-2- tert-butyl-pyridine; 4-methylbenzene- sulfonic acid | 5-Bromo-2- tert-butyl- pyridine (CAS RN: 39919-58-9) | 191.2 [M + H]⁺ |

### Example B.166

### 2-(azetidin-3-yl)-5-[1-(trifluoromethyl)cyclopropyl]pyridine; 4-methylbenzenesulfonic Acid

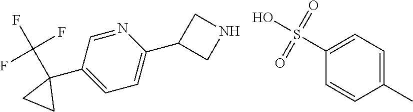

To a mixture of tert-butyl 3-[5-[1-(trifluoromethyl)cyclopropyl]-2-pyridyl]azetidine-1-carboxylate (910 mg, 2.66 mmol) in EtOAc (5 mL) was added p-toluenesulfonic acid (1053 mg, 6.11 mmol). The mixture was stirred at 80° C. for 12 h. The reaction mixture was filtered and the filtrate was concentrated under vacuum to give the title compound (1378 mg, 87% yield) as a white solid. MS (ESI): m/z=243.1 [M−2TsOH+H]⁺

### Step a) Tert-Butyl 3-(5-bromo-2 pyridyl)azetidine-1-carboxylate

To a suspension of Zinc dust (346 mg, 5.3 mmol) in THE (8 mL) were added 1,2-dibromoethane (66.4 mg, 30.4 μL, 0.353 mmol) and TMSCl (44.9 μL, 0.353 mmol) and the suspension was stirred at 60° C. for 15 min. A solution of tert-butyl 3-iodoazetidine-1-carboxylate (614 μL, 3.53 mmol) in DMA (8 mL) was added. The reaction mixture was stored an additional 15 min at 60° C. before cooling to 23° C. 5-bromo-2-iodopyridine (1.05 g, 3.71 mmol), 1,1′-BIS(DIPHENYLPHOSPHINO)FERROCENE-PALLADIUM(II)DICHLORIDE DICHLOROMETHANE COMPLEX (144 mg, 0.177 mmol) and cuprous iodide (34.32 mg, 0.177 mmol) were added and stirring was continued at 80° C. for 2 h. The reaction mixture was diluted with ethyl acetate and water and the mixture was filtered. The filtrate layers were separated. The aqueous layer was extracted twice with ethyl acetate. The organic layers were washed twice with water, dried over MgSO₄, filtered, and evaporated. Purification by FC(SiO₂; heptane/EtOAc) gave the title compound (0.713 g; 61.2%) as a light brown oil. MS (ESI): m/z=257.0 [M-C₄H₈+H]⁺

### Step b) Tert-Butyl 3-[5-[1-(trifluoromethyl)vinyl]-2-pyridyl]azetidine-1-carboxylate

To a solution of 1-(trifluoromethyl)vinylboronic acid hexylene glycol ester (1418 mg, 6.39 mmol) and tert-butyl 3-(5-bromo-2-pyridyl)azetidine-1-carboxylate (2000 mg, 6.39 mmol), POTASSIUM CARBONATE (1765 mg, 12.8 mmol) in 1,4-Dioxane (15 mL) and water (3 mL) was added 1,1′-bis(diphenylphosphino)ferrocene-palladium(II)dichloride dichloromethane complex (521 mg, 0.640 mmol), and the mixture stirred at 100° C. under N₂ atmosphere for 12 h. The reaction mixture was filtered and evaporated. Purification by FC (SiO₂; PE/EtOAc) gave the title compound (1400 mg, 63% yield) as a yellow oil. MS (ESI): m/z=273.1 [M-C₄H₈+H]⁺

### Step c) Tert-Butyl 3-[5-[1-(trifluoromethyl)cyclopropyl]-2 pyridyl]azetidine-1-carboxylate

To a solution of tert-butyl 3-[5-[1-(trifluoromethyl)vinyl]-2-pyridyl]azetidine-1-carboxylate (2160 mg, 7.93 mmol) in THF (20 mL) was added diphenyl(methyl)sulfonium tetrafluoroborate (2972 mg, 10.3 mmol). The suspension was cooled to 0° C. and NaHMDS in THF (1 M solution) (12.7 mL, 12.7 mmol) was added dropwise. The reaction mixture was warmed to 20° C. for and stirred for 12 h. The mixture was a yellow suspension. Purification by FC (SiO₂; PE/EtOAc) gave the title compound (910 mg, 2.66 mmol, 31% yield) as a yellow oil. MS (ESI): m/z=287.0 [M-C₄H₈+H]⁺

### Example B.169

### 3-[3-fluoro-4-(trifluoromethoxy)phenyl]azetidine; 4-methylbenzenesulfonic acid; 4-methylbenzenesulfonic Acid

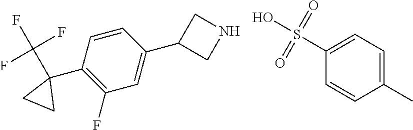

A solution of tert-butyl 3-(3-fluoro-4-(trifluoromethoxy)phenyl)azetidine-1-carboxylate (1.0 g, 2.98 mmol) and 4-methylbenzenesulfonic acid hydrate (596 mg, 3.13 mmol) in Ethyl acetate (10.2 mL) was stirred at reflux for 3 h. The rapidly formed suspension was stirred at 0° C. for 1 h, then filtered. The filter cake was washed with a small volume of ethyl acetate to get the title compound as a colorless solid. MS (ESI): m/z=236.2 [M+H]⁺

### Step a) Tert-Butyl 3-(3-fluoro-4-(trifluoromethoxy)phenyl)azetidine-1-carboxylate

To a stirred suspension of (3-fluoro-4-(trifluoromethoxy)phenyl)boronic acid (2.06 g, 9.18 mmol) in 2-Propanol (16.2 mL) was added tert-butyl 3-iodoazetidine-1-carboxylate (1.3 g, 798 μL, 4.59 mmol) at room temperature, to give a solution. To the mixture was added rac-(1R,2R)-2-aminocyclohexan-1-ol (31.7 mg, 276 μmol), nickel(II) iodide (86.1 mg, 276 μmol) and sodium bis(trimethylsilyl)amide 2 M in THE (4.59 mL, 9.18 mmol) under argon. The mixture was heated in a microwave oven for 1 h at 80° C. The reaction mixture was poured on water and ethyl acetate and the layers were separated. The aqueous layer was extracted twice with ethyl acetate. The organic layers were dried over MgSO₄, filtered, treated with silica gel and evaporated. Purification by FC (SiO₂; PE/EtOAc) gave the title compound as a colorless oil. MS (ESI): m/z=280.2 [M-C₄H₈+H]⁺

### Example B.171

### 5-(azetidin-3 yl)-3-[[1-(trifluoromethyl)cyclopropyl]methyl]-1,2,4-oxadiazole; 4-methylbenzenesulfonic Acid

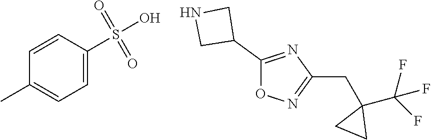 [SMILES: Cc1ccc(S(=O)(=O)O)cc1 ; FC(F)(F)C1(Cc2noc(C3CNC3)n2)CC1]

To a solution of tert-butyl 3-[3-[[1-(trifluoromethyl)cyclopropyl]methyl]-1,2,4-oxadiazol-5-yl]azetidine-1-carboxylate (320 mg, 0.920 mmol) in ethyl acetate (4 mL) was added p-toluenesulfonic acid (190 mg, 1.11 mmol), the mixture was stirred at 80° C. for 12 h. The mixture was cooled to 20° C. and evaporated under reduced pressure to give the residue. To the residue was then added 1 mL ethyl acetate, and a white solid was observed, the mixture was then filtered and washed with EA (5 mL) to give the cake 5-(azetidin-3-yl)-3-[[1-(trifluoromethyl)cyclopropyl]methyl]-1,2,4-oxadiazole; 4-methylbenzenesulfonic acid (335 mg, 86.7% yield) as an off white solid. MS (ESI): m/z=248.2 [M-TsOH+H]⁺

### Step a) N′-hydroxy-2-[1-(trifluoromethyl)cyclopropyl]acetamidine

To a solution of hydroxylamine hydrochloride (839 mg, 12.1 mmol) and 2-[1-(trifluoromethyl)cyclopropyl]acetonitrile (CAS: 1454690-79-9) (900 mg, 6.04 mmol) in methanol (7 mL), water (7 mL), and sodium carbonate (1280 mg, 12.1 mmol) was added. The mixture was stirred at 50° C. for 12 h. The mixture was filtered, and the filtrate was concentrated under vacuum to remove the ethanol, then the residual solution was extracted with EtOAc (50 mL twice), the combined organic phase was dried over Na₂SO₄, concentrated to give N′-hydroxy-2-[1-(trifluoromethyl)cyclopropyl]acetamidine (700 mg, 63.67% yield) as light yellow solid, which was used directly without further purification. MS (ESI): m/z=183.1 [M+H]⁺

### Step b) O3-[(Z)-[1-amino-2-[1-(trifluoromethyl)cyclopropyl]ethylidene]amino]O1-tert-butyl azetidine-1,3-dicarboxylate

To a solution of DIPEA (1441 mg, 11.2 mmol), HATU (1696 mg. 4.46 mmol) and 1-Boc-azetidine-3-carboxylic acid (CAS: 142253-55-2) (928 mg, 4.61 mmol) in DCM (16 mL) was stirred for 5 mm, then N′-hydroxy-2-[1-(trifluoromethyl)cyclopropyl]acetamidine (700 mg, 3.84 mmol) was added and stirred at 20° C. for 12 h. The reaction mixture was poured into H₂O (50 mL), extracted with EtOAc (50 mL×3), and purified with reversed phase column and lyophilized to give O3-[(Z)-[1-amino-2-[1-(trifluoromethyl)cyclopropyl]ethylidene]amino] O1-tert-butyl azetidine-1,3-dicarboxylate (1100 mg, 78% yield) as a light brown solid. MS (ESI): m/z=310.1 [M-C₄H₈+H]⁺

### Step c) Tert-Butyl 3-[3-[[1-(trifluoromethyl)cyclopropyl]methyl]-1,2,4-oxadiazol-5-yl]azetidine-1-carboxylate

A solution of O3-[(Z)-[1-amino-2-[1-(trifluoromethyl)cyclopropyl]ethylidene]amino] O1-tert-butyl azetidine-1,3-dicarboxylate (360 mg, 0.990 mmol) in DMF (18 mL) was stirred at 130° C. for 12 h. The reaction mixture was purified with reversed phase column (0.225% v/vFA) and lyophilized to give tert-butyl 3-[3-[[1-(trifluoromethyl)cyclopropyl]methyl]-1,2,4-oxadiazol-5-yl]azetidine-1-carboxylate (320 mg, 93.5% yield) as a yellow oil. MS (ESI): m/z=292.1 [M-C₄H₈+H]⁺

### Example B. 172

### 3-(azetidin-3-yl)-5-[1-(trifluoromethyl)cyclopropyl]-1,2,4-oxadiazole; 4-methylbenzenesulfonic Acid Salt

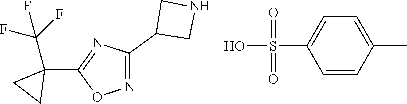 [SMILES: FC(F)(F)C1(c2nc(C3CNC3)no2)CC1 ; Cc1ccc(S(=O)(=O)O)cc1]

To a solution of tert-butyl 3-[5-[1-(trifluoromethyl)cyclopropyl]-1,2,4-oxadiazol-3-yl]azetidine-1-carboxylate (1200 mg, 3.6 mmol) in ethyl acetate (20 mL) was added p-toluenesulfonic acid (744 mg, 4.32 mmol), the mixture was stirred at 80° C. for 12 h. The mixture was cooled to room temperature and concentrated, to give the title compound (1315 mg, 89.7% yield) as a brown waxy solid. MS (ESI): m/z=234.4 [M-C₇H₈O₃S+H]⁺

### Step a) Tert-Butyl 3-(N-hydroxycarbamimidoyl)azetidine-1-carboxylate

To a solution of hydroxylamine hydrochloride (1.53 g, 22.0 mmol) and 1-Boc-3-cyanoazetidine (2.0 g, 11.0 mmol) in methanol (20 mL) and water (20 mL) was added sodium carbonate (2.33 g, 22.0 mmol) and the mixture was stirred at 50° C. for 12 h. The mixture was filtered, and the filtrate was concentrated under vacuum to remove the ethanol, then the residual mixture was extracted with EtOAc (50 mL×2). The combined organic phase was dried over Na₂SO₄ and concentrated to give the title compound (1.8 g, 76.2% yield) as a light yellow solid. MS (ESI): m/z=160.2 [M-C₄H₈+H]⁺

### Step b) 3-[(Z)—N′-[1-(trifluoromethyl)cyclopropanecarbonyl]oxycarbamimidoyl]azetidine-1-carboxylate

To a solution of 1-(trifluoromethyl)cyclopropane-1-carboxylic acid (1432 mg, 9.29 mmol), DIPEA (3603 mg, 27.9 mmol) and HATU (4240 mg, 11.2 mmol) in DCM (40 mL) was added tert-butyl 3-(N-hydroxycarbamimidoyl)azetidine-1-carboxylate (2000 mg, 9.29 mmol), then the reaction mixture was stirred at 20° C. for 16 h. The mixture was evaporated and purified by reverse flash chromatography (FA) to give the title compound (2600 mg, 79.7% yield) as light brown oil. MS (ESI): m/z=296.3 [M-C₄H₈+H]⁺

### Step c) Tert-Butyl 3-[5-[1-(trifluoromethyl)cyclopropyl]-1,2,4-oxadiazol-3 yl]azetidine-1-carboxylate

To a solution of tert-butyl 3-[(Z)—N′-[1-(trifluoromethyl) cyclopropanecarbonyl]oxycarbamimidoyl]azetidine-1-carboxylate (1500 mg, 4.27 mmol) in ethanol (37.5 mL) and water (37.5 mL) was added KOAc (838 mg, 8.54 mmol). The mixture was stirred at 80° C. for 12 h, then the mixture was concentrated and diluted with EtOAc (50 mL), washed with water and brine, dried over Na₂SO₄ and concentrated. The residue was purified by reverse flash chromatography to give the title compound (1250 mg, 87.8% yield) as light yellow oil. MS (ESI): m/z=278.4 [M-C₄H₈+H]⁺

### Example B. 174

### 3-(2-chloro-4-isopropylsulfonyl-phenyl)azetidine; 4-methylbenzenesulfonic Acid

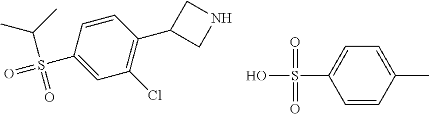 [SMILES: CC(C)S(=O)(=O)c1ccc(C2CNC2)c(Cl)c1 ; Cc1ccc(S(=O)(=O)O)cc1]

To a solution of 3-(2-chloro-4-isopropylsulfonyl-phenyl)azetidine-1-carboxylic acid ten-butyl ester (124 mg, 0.322 mmol) in ethyl acetate (1.1 mL) was added 4-methylbenzenesulfonic acid acid monohydrate (67.31 mg, 0.354 mmol) and the solution was stirred at reflux in a sealed tube for 1 h. After cooling down, the suspension was filtered. The filter cake was washed with a small volume of ethyl acetate to get the desired product as a colorless solid (0.131 g, 87%). MS (ESI): m/z=274.2 [M+H]⁺

### Step a) 3-(2-chloro-4-fluoro-phenyl)azetidine-1-carboxylic Acid Tert-Butyl Ester

To a 100 mL flask equipped with a stir bar were added (IR[dF(CF₃)PPY]₂(DTBPY))PF₆ (161 mg, 0.143 mmol), tert-butyl 3-bromoazetidine-1-carboxylate (3.38 g, 14.3 mmol), 1,1,1,3,3,3-hexamethyl-2-(trimethylsilyl)trisilane (4.4 mL, 14.3 mmol), 1,1,1,3,3,3-hexamethyl-2-(trimethylsilyl)trisilane (4.4 mL, 14.3 mmol) and anhydrous sodium carbonate (3.04 g, 28.6 mmol). The vial was sealed and placed under Ar before DME (29 mL) was added. To a separate vial were added NICKEL(11) CHLORIDE ETHYLENE GLYCOL DIMETHYL ETHER COMPLEX (15.74 mg. 0.072 mmol) and 4,4′-di-tert-butyl-2,2′-bipyridine (19.22 mg, 0.072 mmol), under Ar. This vial was sealed, purged with Ar, treated with DME (4 mL), and sonicated for 5 min, before being added to the main reaction vial. The mixture was stirred and irradiated with a 420 nm lamp for 64 h, before being filtered and evaporated. Purification by FC (SiO₂; heptane/EtOAc) gave the title compound (2.02 g; 39.5%) as a light yellow oil. MS (ESI): m/z=230.1 [M-tBu+H]⁺

### Step b) 3-[2-chloro-4-(isopropylthio)phenyl]azetidine-1-carboxylic Acid Tert-Butyl Ester

To a solution of 3-[2-chloro-4-fluoro-phenyl)azetidine-1-carboxylic acid tert-butyl ester (320 mg, 0.896 mmol) in dimethyl sulfoxide (5 mL) was added sodium; propane-2-thiolate (87.9 mg, 0.896 mmol) and the mixture was heated to 100° C. overnight. Another batch of sodium; propane-2-thiolate (87.9 mg, 0.896 mmol) was added and stirring was continued overnight at 100° C. The reaction mixture was poured on water and ethyl acetate and the layers were separated. The aqueous layer was extracted with ethyl acetate. The organic layers were washed with water, dried over MgSO₄, filtered, treated with silica and evaporated. The crude product was purified by silica gel chromatography eluting with a gradient of n-heptane: ethyl acetate (100:0 to 70:30) to get the desired product as a colorless gum (0.173 g; 54%). MS (ES): m/z=286.1 [M-C₄H₈+H]+.

### Step c) 3-(2-chloro-4-isopropylsulfonyl phenyl)azetidine-1-carboxylic Acid Tert-Butyl Ester

To a solution of 3-[2-chloro-4-(isopropylthio)phenyl]azetidine-1-carboxylic acid tert-butyl ester (173 mg, 0.481 mmol) in dichloromethane (1.5 mL) at RT was added carefully 3-chloroperoxybenzoic acid (269 mg, 1.2 mmol) and the rapidly formed suspension was stirred at RT for 2.75 h. The suspension was filtered. The filter cake was washed with a small volume of DCM. The filtrate was washed twice with aqueous half-saturated NaHCO₃ solution. The aqueous layers were extracted twice with DCM. The organic layers were dried over MgSO₄, filtered, treated with silica gel and evaporated. The reaction mixture was poured on half-saturated aqueous NH₄Cl solution and ethyl acetate and the layers were separated. The aqueous layer was extracted twice with ethyl acetate. The organic layers were washed twice with half-saturated aqueous NH₄Cl solution, dried over MgSO₄, filtered, treated with isolute and evaporated. Purification by FC (SiO₂; heptane/EtOAc) gave the title compound (0.124 g, 67%) as a colorless gum. MS (ESI): m/z=318.1 [M+H]⁺

### Example B.179

### 5-(azetidin-3-yl)-N-[[1-(trifluoromethyl)cyclopropyl]methyl]pyridin-2-amine; 4-methylbenzenesulfonic Acid

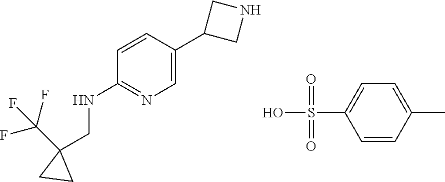 [SMILES: FC(F)(F)C1(CNc2ccc(C3CNC3)cn2)CC1 ; Cc1ccc(S(=O)(=O)O)cc1]

To a stirred solution of tert-butyl 3-[6-[[1-(trifluoromethyl)cyclopropyl]methylamino]-3-pyridyl]azetidine-1-carboxylate (5.5 g, 14.8 mmol) in EtOAc (300 mL), p-toluenesulfonic acid monohydrate (7.04 g, 37.0 mmol) was added. Then RM was stirred at 50° C. for 24 h. The reaction mixture was evaporated in vacuo and obtained residue was stirred with TBME (300 mL) for 12 h. The obtained precipitate was filtered, washed with TBME (2×200 mL) and dried to give the title compound (5318 mg, 55% yield) as a light yellow solid. MS (ESI): m/z=272.2 [M+H]⁺

### Step a) Tert-Butyl 3-(6-bromo-3-pyridyl)azetidine-1-carboxylate

The stirred mixture of tert-butyl 3-(p-tolylsulfonylhydrazono)azetidine-1-carboxylate (CAS: 1510865-66-3) (68.0 g, 200 mmol), 2-bromopyridine-5-boronic acid (53.8 g, 266 mmol) and potassium carbonate (41.5 g, 301 mmol) in dry 1,4-Dioxane (2800 mL) were refluxed for 24 h (Argon). Then obtained precipitate was filtered off and filtrate was evaporated to dryness. The obtained residue was partioned between TBME (2000 mL) and water (500 mL). The organic layer was washed with brine (100 mL), dried over Na₂SO₄ and evaporated in vacuum. The crude product was purified by flash chromatography to obtain the title compound (13.8 g, 44.0 mmol, 21% yield) as a light yellow oil. MS (ESI): m/z=313.0 [M+H]⁺

### Step b) Tert-Butyl 3-[6-[[1-(trifluoromethyl)cyclopropyl]methylamino]-3 pyridyl]azetidine-1-carboxylate

The mixture of tert-butyl 3-(6-bromo-3-pyridyl)azetidine-1-carboxylate (7.0 g, 22.4 mmol), [1-(trifluoromethyl)cyclopropyl]methanamine; hydrochloride (5.89 g, 33.5 mmol), tris(dibenzylideneacetone)dipalladium (1.02 g, 1.12 mmol), XantPhos (1.03 g, 1.79 mmol) and sodium tert-butoxide (6.44 g, 67.1 mmol) was sealed and stirred in degassed Toluene (100 mL) at 100° C. for 24 h (Argon atmosphere). Then RM was cooled to RT and filtered through a pad of SiO₂, washed with toluene (300 mL) and concentrated in vacuo. Purification by FC (SiO₂; hexane/MTBE) gave the title compound (5.5 g, 63% yield) as orange crystals. MS (ESI): m/z=372.2 [M+H]⁺

In analogy to Example B. 179, the following building blocks were generated using the relevant commercial building blocks.

| | | | | MS, |
|---|---|---|---|---|
| | | | Building | ESI: |
| Ex. | Structure | Systematic Name | Blocks | m/z |
| B.183 | 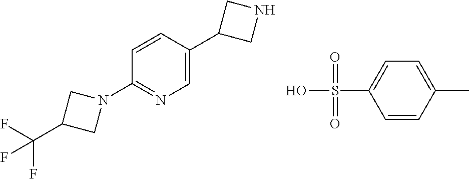 [SMILES: FC(F)(F)C1CN(c2ccc(C3CNC3)cn2)C1 ; Cc1ccc(S(=O)(=O)O)cc1] | 5-(azetidin-3-y1)-2- [3- (trifluoromethyl) azetidin-1-yl]pyridine; di 4- methylbenzenesulfonic acid | 3- (trifluoromethyl) azetidine; hydrochloride | 258.2 [M − 2TsOH + H]⁺ |
| | * Xphos catalyst used in Step b) | | | |

### Example B. 180

### 5-(azetidin-3-yl)-N-[[1-(trifluoromethyl)cyclopropyl]methyl]pyrazin-2-amine; di 4-methylbenzenesulfonic Acid

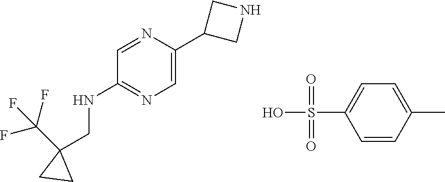 [SMILES: FC(F)(F)C1(CNc2cnc(C3CNC3)cn2)CC1 ; Cc1ccc(S(=O)(=O)O)cc1]

A mixture of tert-butyl 3-[5-[[1-(trifluoromethyl)cyclopropyl]methylamino]pyrazin-2-yl]azetidine-1-carboxylate (1.9 g, 5.1 mmol) and p-toluenesulfonic acid (1142 mg, 6.63 mmol) in EtOAc (20 mL) was stirred at 80° C. for 12 h. A further addition of p-toluenesulfonic acid (87.9 mg, 0.510 mmol) was made, and the mixture was stirred for another 12 h at 80° C. The reaction was concentrated under vacuum to give a residue. To the residue was added 80 mL water and the mixture was lyophilized to the title compound (2.38 g, 75% yield) as a yellow solid. MS (ESI): m/z=273.2 [M−2TsOH+H]⁺

### Step a) Tert-Butyl 3-(5-bromopyrazin-2 yl)azetidine-1-carboxylate

To a mixture of zinc (4131 mg, 63.2 mmol) in THF (96 mL) was added 1,2-dibromoethane (791 mg, 4.21 mmol) and CHLOROTRIMETHYLSILANE (458 mg, 4.21 mmol). The mixture was heated to 60° C. and stirred for 15 min. Then a mixture of 1-BOC-3-iodoazetidine (12.5 g, 44.2 mmol) in DMA (96 mL) was added. The mixture was stirred for another 15 min. The mixture was cooled to 20° C. and 2-bromo-5-iodopyrazine (12.0 g, 42.1 mmol), 1,1-BIS(DIPHENYLPHOSPHINO)FERROCENE-PALLADIUM(II)DICHLORIDEDICHLOROMETHANECOMPLEX (1720 mg, 2.11 mmol) and COPPER(I) IODIDE (0.07 mL, 2.11 mmol) were added. The mixture was heated to 80° C. and stored for 12 h. The mixture was added to 200 mL water and extracted with EtOAc (200 mL×3). The combined organic phases were evaporated and purified by FC (SiO₂; PE/EtOAc), to give the title compound (4.9 g, 37% yield) as a white solid. MS (ESI): m/z=258.1 [M-C₄H₈+H]⁺

### Step b) Tert-Butyl 3-[5-[[1-(trifluoromethyl)cyclopropyl]methylamino]pyrazin-2-yl]azetidine-1-carboxylate

To a solution of [1-(trifluoromethyl)cyclopropyl]methanamine; hydrochloride (1667 mg, 9.49 mmol), tert-butyl 3-(5-bromopyrazin-2-yl)azetidine-1-carboxylate (3000 mg, 9.55 mmol) and sPhos-Pd-G3 (836 mg, 0.950 mmol) in t-Amyl-OH (55.5 mL) was added tBuONa 1M THF SOLUTION (14.3 mL, 28.6 mmol) under N₂ atmosphere, the mixture was degassed with N₂ for 1 min and stirred under N₂ atmosphere at 100° C. for 12 h. The mixture was evaporated and purified by RP-HPLC, to give the title compound (1.9 g, 53% yield) as a yellow solid. MS (ESI): m/z=373.1 [M+H]⁺

In analogy to Example B. 180, the following building blocks were generated using the relevant commercial building blocks. In some cases alternative acids (e.g. TFA, HCl were used for the final deprotection).

| | | | | MS, |
|---|---|---|---|---|
| | | | Building | ESI: |
| Ex. | Structure | Systematic Name | Blocks | m/z |
| B.181 | 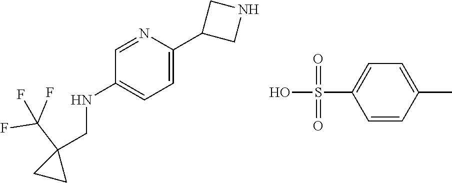 [SMILES: FC(F)(F)C1(CNc2ccc(C3CNC3)nc2)CC1 ; Cc1ccc(S(=O)(=O)O)cc1] | 6-(azetidin-3-yl)-N- [[1- (trifluoromethyl) cyclopropyl] methyl]pyridin- 3-amine; di 4- methylbenzenesulfonic acid | 2,5- dibromopyridine; [1- (trifluoromethyl) cyclopropyl] methanamine hydrochloride | 272.2 [M − 2TsOH + H]⁺ |
| | * (BuXPhos-Pd-G3 catalyst used in Step b) | | | |
| B.182 | 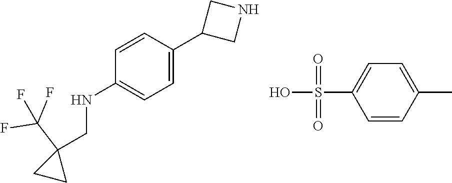 [SMILES: FC(F)(F)C1(CNc2ccc(C3CNC3)cc2)CC1 ; Cc1ccc(S(=O)(=O)O)cc1] | 4-(azetidin-3-yl)-N- [[1- (trifluoromethyl) cyclopropyl\|methyl] aniline; 4- methylbenzenesulfonic acid | Step b) 1- Boc-3-(4- bromophenyl) azetidine (CAS: 1203681- 52-0); [1- (trifluoromethyl) cyclopropyl] methanamine hydrochloride | 271.5 [M − TsOH + H]⁺ |
| | * tBuXPhos-Pd-G3 catalyst used | | | |
| | in Step b) | | | |
| B.184 | 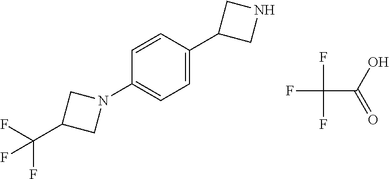 [SMILES: FC(F)(F)C1CN(c2ccc(C3CNC3)cc2)C1 ; O=C(O)C(F)(F)F] | 1-[4-(azetidin-3- yl)phenyl]-3- (trifluoromethyl)azet idine; trifluoroacetic acid | Step b) 1- Boc-3-(4- bromophenyl) azetidine (CAS: 1203681- 52-0); 3- (trifluoromethyl) azetidine hydrochloride | 257.4 [M − TFA + H]⁺ |
| | * Pd(OAc)₂/ (R)-(+)-2,2′- | | | |
| | bis(diphenylphosphino)-1,1'- | | | |
| | binaphthalenecatalyst/ligand used | | | |
| | in Step b) | | | |
| B.186 | 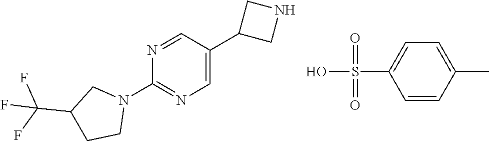 [SMILES: FC(F)(F)C1CCN(c2ncc(C3CNC3)cn2)C1 ; Cc1ccc(S(=O)(=O)O)cc1] | 5-(azetidin-3-y1)-2- [3- (trifluoromethyl) pyrrolidin-1- yl]pyrimidine; 4- methylbenzenesulfonic acid | 2-chloro-5- iodo- pyrimidine; 3- (trifluoromethyl) pyrrolidine hydrochloride | 273.1 [M − TsOH + H]⁺ |
| | * Xphos/ | | | |
| | tris(dibenzylideneacetone) | | | |
| | dipalladium (0) chloroform adduct | | | |
| | used in Step b) | | | |
| B.190 | 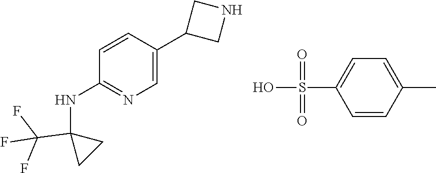 [SMILES: FC(F)(F)C1(Nc2ccc(C3CNC3)cn2)CC1 ; Cc1ccc(S(=O)(=O)O)cc1] | 5-(azetidin-3-yl)-N- [1- (trifluoromethyl) cyclopropyl]pyridin-2- amine; 4- methylbenzenesulfonic acid | Step b) 5- (1-Boc-3- azetidinyl)-2- chloropyridine (CAS: 870689-19-3); 1- (trifluoromethyl) cyclopropanamine hydrochloride (CAS: 112738-67-7) | 258.2 [M − TsOH + H]⁺ |
| | * tBuXPhos-Pd-G3 catalyst used in Step b) | | | |
| B.191 | 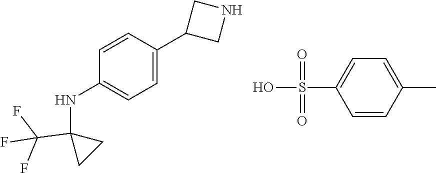 [SMILES: FC(F)(F)C1(Nc2ccc(C3CNC3)cc2)CC1 ; Cc1ccc(S(=O)(=O)O)cc1] | 4-(azetidin-3-y1)-N- [1- (trifluoromethyl) cyclopropyl]aniline; 4- methylbenzenesulfonic acid | Step b) 1- Boc-3-(4- bromophenyl) azetidine (CAS: 1203681- 52-0); 1- (trifluoromethyl) cyclopropanamine hydrochloride | 257.4 [M − TsOH + H]⁺ |
| | * tBuXPhos-Pd-G3 catalyst used | | | |
| | in Step b) | | | |
| B.495 | 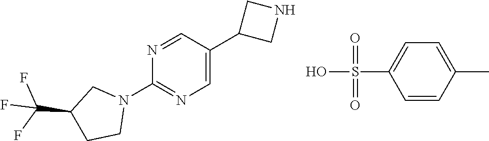 [SMILES: FC(F)(F)[C@@H]1CCN(c2ncc(C3CNC3)cn2)C1 ; Cc1ccc(S(=O)(=O)O)cc1] | 5-(azetidin-3-y1)-2- [(3R)-3- (trifluoromethyl) pyrrolidin-1- yl]pyrimidine;4- methylbenzenesulfonic acid | 2-chloro-5-iodo- pyrimidine;3- (trifluoromethyl) pyrrolidine hydrochloride | 273.2 [M − TsOH + H]+ |
| | * Xphos/ | | | |
| | tris(dibenzylideneacetone) | | | |
| | dipalladium (0) chloroform adduct | | | |
| | used in Step b); arbitrary | | | |
| | assignment of enantiomers, | | | |
| | separated by SFC after Step b) | | | |
| B.496 | 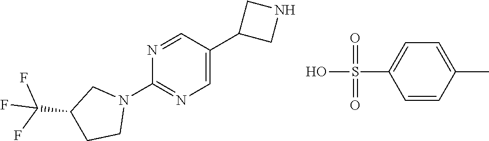 [SMILES: FC(F)(F)[C@H]1CCN(c2ncc(C3CNC3)cn2)C1 ; Cc1ccc(S(=O)(=O)O)cc1] | 5-(azetidin-3-y1)-2- [(3,5)-3- (trifluoromethyl) pyrrolidin-1- yl]pyrimidine;4- methylbenzenesulfonic acid | 2-chloro-5- iodo- pyrimidine; 3- (trifluoromethyl) pyrrolidine hydrochloride | 273.2 [M − TsOH + H]⁺ |
| | * Xphos/ | | | |
| | tris(dibenzylideneacetone) | | | |
| | dipalladium (0) chloroform adduct | | | |
| | used in Step b); arbitrary | | | |
| | assignment of enantiomers, | | | |
| | separated by SFC after Step b) | | | |

### Example B.185

### 5-(azetidin-3-yl)-2-[13-(trifluoromethyl)azetidin-1-yl]pyrimidine; di 4-methylbenzenesulfonic Acid

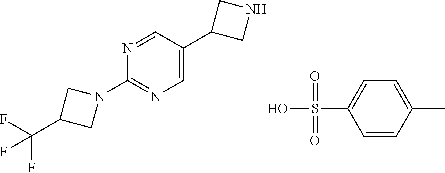 [SMILES: FC(F)(F)C1CN(c2ncc(C3CNC3)cn2)C1 ; Cc1ccc(S(=O)(=O)O)cc1]

A mixture of p-toluenesulfonic acid (1474 mg, 8.56 mmol), tert-butyl 3-[2-[3-(trifluoromethyl)azetidin-1-yl]pyrimidin-5-yl]azetidine-1-carboxylate (2.36 g, 6.59 mmol) in EtOAc (20 mL) was stirred at 80° C. for 12 h. The mixture was filtered and cake was concentrated to give the title compound (3.49 g, 88% yield) as a white solid. MS (ESI): m/z=259.2 [M−2TsOH+H]⁺

### Step a) 5-bromo-2-[3-(trifluoromethyl)azetidin-1-yl]pyrimidine

A solution of DIPEA (4.8 g, 37.1 mmol), 3-(trifluoromethyl)azetidine; hydrochloride (2.0 g, 12.4 mmol) and 5-bromo-2-fluoropyrimidine (2.63 g, 14.9 mmol) in DMSO (15 mL) was stirred at 100° C. for 16 h.

The aqueous phase was extracted with ethyl acetate (100 mL×3). The combined organic phase was washed with brine (100 mL×3), dried with anhydrous Na₂SO₄, filtered and concentrated in vacuum. Purification by FC (SiO₂; PE/EtOAc) gave the title compound (3.24 g, 93% yield) as a yellow solid. ¹H NMR (400 MHz, CHLOROFORM-d) δ=8.35 (s, 2H), 4.33-4.25 (m, 2H), 4.23-4.16 (m, 2H), 3.46-3.31 ppm (m, 1H).

### Step b) Tert-Butyl 3-[2-[3-(trifluoromethyl)azetidin-1-yl]pyrimidin-5-yl]azetidine-1-carboxylate

To a 250 mL vial equipped with a stir bar was added tert-butyl 3-bromoazetidine-1-carboxylate (3482 mg, 14.8 mmol), 5-bromo-2-[3-(trifluoromethyl)azetidin-1-yl]pyrimidine (3200 mg, 11.4 mmol), Ir[dF(CF₃)ppy]₂(dtbbpy)PF₆ (127 mg, 0.110 mmol), NiCl₂·dtbbpy (22.6 mg, 0.060 mmol), Na₂CO₃ (2405 mg, 22.7 mmol), TTMSS (2822 mg, 11.4 mmol) in DME (100 mL). The vial was sealed and placed under nitrogen. The reaction was stirred and irradiated with a 34 W blue LED lamp (7 cm away), with cooling fan to keep the reaction temperature at 25° C. for 14 h. The mixture was filtered and evaporated. Purification by RP-HPLC gave the title compound (2.4 g, 6.7 mmol, 59% yield) as a white solid. MS (ESI): m/z=359.3 [M+H]⁺

In analogy to Example B. 180, the following building blocks were generated using the relevant commercial building blocks. In some cases, alternative conditions for the SNAr reaction in Step a) were used such as K₂CO₃, DMF, 110° C. microwave.

| | | | | MS, |
|---|---|---|---|---|
| | | | Building | ESI: |
| Ex. | Structure | Systematic Name | Blocks | m/z |
| B.187 | 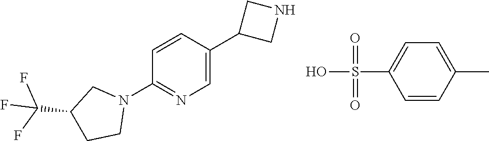 [SMILES: FC(F)(F)[C@H]1CCN(c2ccc(C3CNC3)cn2)C1 ; Cc1ccc(S(=O)(=O)O)cc1] | 5-(azetidin-3-yl)-2- [(3S)-3- (trifluoromethyl) pyrrolidin-1- yl]pyridine;4- methylbenzenesulfonic acid | 5-bromo-2- fluoropyridine; 3- (trifluoromethyl) pyrrolidine hydrochloride | 272.1 [M − TsOH + H]⁺ |
| | *arbitrary assignment of | | | |
| | stereochemistry, separated by | | | |
| | chiral SFC after Step b) | | | |
| B.188 | 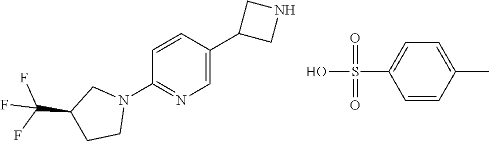 [SMILES: FC(F)(F)[C@@H]1CCN(c2ccc(C3CNC3)cn2)C1 ; Cc1ccc(S(=O)(=O)O)cc1] | 5-(azetidin-3-y1)-2- [(3R)-3- (trifluoromethyl) pyrrolidin-1- yl]pyridine;4- methylbenzenesulfonic acid | 5-bromo-2- fluoropyridine; 3- (trifluoromethyl) pyrrolidine hydrochloride | 272.1 [M − 2TsOH + H]⁺ |
| | *arbitrary assignment of | | | |
| | stereochemistry, separated by | | | |
| | chiral SFC after Step b) | | | |
| B.189 | 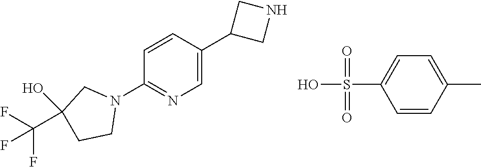 [SMILES: OC1(C(F)(F)F)CCN(c2ccc(C3CNC3)cn2)C1 ; Cc1ccc(S(=O)(=O)O)cc1] | 1-[5-(azetidin-3-y1)- 2-pyridyl]-3- (trifluoromethyl) pyrrolidin-3-ol; 4- methylbenzenesulfonic acid | 5-bromo-2- fluoropyridine; 3-(trifluoromethyl) pyrrolidin-3-ol hydrochloride | 288.1 [M − 2TsOH + H]⁺ |
| B.192 | 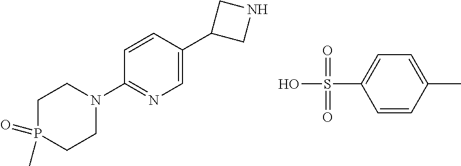 [SMILES: CP1(=O)CCN(c2ccc(C3CNC3)cn2)CC1 ; Cc1ccc(S(=O)(=O)O)cc1] | 1-[5-(azetidin-3-yl)- 2-pyridyl]-4-methyl- 1,4λ⁵- azaphosphinane 4- oxide; 4- methylbenzenesulfonic acid | 5-bromo-2- fluoropyridine; 4- methyl- 1,4λ⁵- azaphosphinane 4-oxide hydrochloride (CAS: 945459-80-3) | 266.1 [M − 2TsOH + H]⁺ |
| B.196 | 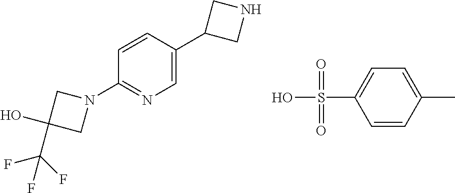 [SMILES: OC1(C(F)(F)F)CN(c2ccc(C3CNC3)cn2)C1 ; Cc1ccc(S(=O)(=O)O)cc1] | 1-[5-(azetidin-3-y])- 2-pyridyl]-3- (trifluoromethyl) azetidin-3-ol; di 4- methylbenzenesulfonic acid | 5-bromo-2- fluoropyridine; 3- (trifluoromethyl) azetidin-3-ol hydrochloride | 274.2 [M − 2TsOH + H]⁺ |

### Example B.193

### 4-[5-(azetidin-3-yl)-2-pyridyl]-1,4-thiazinane 1,1-dioxide; trifluoroacetic Acid

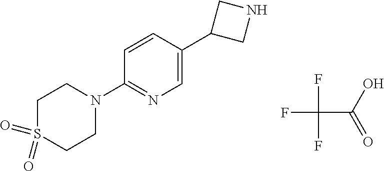 [SMILES: O=S1(=O)CCN(c2ccc(C3CNC3)cn2)CC1 ; O=C(O)C(F)(F)F]

A solution of 3-[6-(1,1-diketo-1,4-thiazinan-4-yl)-3-pyridyl]azetidine-1-carboxylic acid tert-butyl ester (160 mg, 0.435 mmol) in dichloromethane (5 mL) was treated with TFA (336 μL, 4.35 mmol) at 25° C. The mixture was stirred for 18 h at this temperature, before being evaporated. The material was used directly in the next step without further purification. MS (ESI): m/z=268.2 [M-TFA+H]⁺

### Step a): 3-(6-fluoro-3 pyridyl)azetidine-1-carboxylic Acid Tert-Butyl Ester

To a 500 mL flask equipped with a stir bar were added 5-bromo-2-fluoro-pyridine (CAS RN: 766-11-0; 6 g, 34.09 mmol), tert-butyl 3-bromoazetidine-1-carboxylate (8.85 g, 37.5 mmol), (IR[dF(CF₃)PPY]₂(DTBPY))PF₆ (382.5 mg, 0.341 mmol), TRIS(TRIMETHYLSILYL)SILANE (8.48 g, 10.52 mL, 34.09 mmol) and anhydrous sodium carbonate (5.42 g, 51.14 mmol), at 23° C. under Ar. DME (320 mL) was added and the mixture was sparged with Ar for 10 min, before being sealed. To a separate vial (flushed with argon) were added NICKEL(II) CHLORIDE ETHYLENE GLYCOL DIMETHYL ETHER COMPLEX (37.45 mg, 0.170 mmol), 4,4′-DI-TERT-BUTYL-2,2′-BIPYRIDINE (45.75 mg, 0.170 mmol) and DME (20 mL). The vial was sealed, sparged with Ar, and sonicated for 10 min, before being added to the matin reaction vial. The reaction was stirred under Ar and irradiated with a 465 nm lamp for 20 h, before being filtered and evaporated. Purification by FC (SiO₂; heptane/EtOAc) gave the title compound (5.69 g, 59.58%) as an orange viscous oil. MS (ESI): m/z=253.1 [M+H]⁺

### Step b): 3-(6-thiomorpholino-3-pyridyl)azetidine-1-carboxylic Acid Tert-Butyl Ester

A solution of thiomorpholine (1.84 g, 17.84 mmol) in DMSO (7.5 mL) was treated with DIPEA (3.05 mL, 17.84 mmol) and 3-(6-fluoro-3-pyridyl)azetidine-1-carboxylic acid tert-butyl ester (450 mg, 1.78 mmol), at 23° C. under Ar. The mixture was heated to 120° C. and stirred at this temperature for 78 h, before being cooled down to 23° C. The solution was diluted with EtOAc, and the organic phase washed with water (2×), brine (1×), dried over Na₂SO₄, and evaporated. Purification by FC (SiO₂; heptane/EtOAc) gave the title compound (560 mg, 74.87%) as a light brown oil. MS (ESI): m/z=336.3 [M+H]⁺

### Step c): 3-[6-(1,1-diketo-1,4-thiazinan-4 yl)-3 pyridyl]azetidine-1-carboxylic Acid Tert-Butyl Ester

A solution of 3-(6-thiomorpholino-3-pyridyl)azetidine-1-carboxylic acid tert-butyl ester (560 mg, 1.34 mmol) in dichloromethane (16 mL) was treated with 3-chloroperoxybenzoic acid (460.93 mg, 2.67 mmol), at 0° C. under Ar. The mixture was stirred for 1 h at this temperature, before being allowed to warm up to 23° C. over 16 h. The mixture was then diluted with DCM and washed with saturated aqueous Na₂S₂O₃. The organic layer was dried over Na₂SO₄, filtered, and evaporated. Purification by FC (SiO₂; heptane/EtOAc) gave the title compound (160 mg, 32.6%) as a colorless oil. MS (ESI): m/z=368.2 [M+H]⁺

### Example B.197

### 1-[4-(Azetidin-3-yl)phenyl]-3,5-diisopropyl-pyrazole; 4-methylbenzenesulfonic Acid

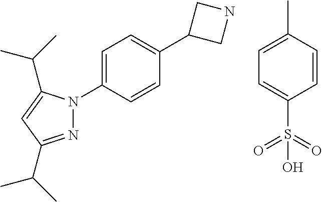

To a solution of 3-[4-(3,5-diisopropylpyrazol-1-yl)phenyl]azetidine-1-carboxylic acid tert-butyl ester (245 mg, 0.639 mmol) in EtOAc (3.71 mL) was added 4-methylbenzenesulfonic acid hydrate (249.09 mg, 1.31 mmol). At 23° C. The mixture was stirred at reflux for 2 h, before being allowed to cool down. The mixture was evaporated, to give the title compound (0.419 g; 87.3%) as a light yellow foam. MS (ESI): m/z=284.3 [M+H]⁺

### Step a) 1-(4-Bromophenyl)-3,5-diisopropyl-pyrazole

A solution of 3,5-diisopropyl-1H-pyrazole (CAS RN: 17536-00-4; 220 mg, 1.45 mmol) in dichloromethane (14.36 mL) was treated with copper (II) acetate (354.31 mg, 1.95 mmol), TEA (0.4 mL, 2.89 mmol) and 4-bromophenylboronic acid (435.33 mg, 2.17 mmol), at 23° C. The mixture was stirred for 48 h at this temperature, before being filtered. The filtrate was washed with DCM and evaporated. Purification by FC (SiO₂; heptane/EtOAc) gave the title compound (0.274 g, 58.6%) as a colorless oil. MS (ESI): m/z=309.1 [M+H]⁺

### Step b) Tert-Butyl 3-[4-(3,5-diisopropylpyrazol-1 yl)phenyl]azetidine-1-carboxylate

To a 20 mL vial equipped with a stir bar were added (IR[DF(CF3)PPY]2(DTBPY))PF6 (9.97 mg, 0.009 mmol), 1-(4-bromophenyl)-3,5-diisopropyl-pyrazole (273 mg, 0.889 mmol), tert-butyl 3-bromoazetidine-1-carboxylate (218.09 μL, 1.33 mmol), TRIS(TRIMETHYLSILYL)SILANE (274.14 μL, 0.889 mmol) and anhydrous sodium carbonate (188.36 mg, 1.78 mmol). The vial was sealed and placed under argon before DME (7.56 mL) was added. To a separate vial were added NICKEL(II) CHLORIDE ETHYLENE GLYCOL DIMETHYL ETHER COMPLEX (1.95 mg, 0.009 mmol) and 4,4′-DI-TERT-BUTYL-2,2′-BIPYRIDINE (2.39 mg, 0.009 mmol). The precatalyst vial was sealed, sparged with argon, and treated with DME (3.02 mL). The precatalyst vial was sonicated for 5 min, after which 1.51 mL of it was syringed into the reaction vessel. The mixture was stirred and irradiated with a 420 nm lamp for 16 h (75% intensity), before being filtered and evaporated. Purification by FC (SiO₂; heptane/EtOAc) gave the title compound (0.246 g; 67.2%) as a light yellow oil. MS (ESI): m/z=384.3 [M+H]

In analogy to Example B. 197, Examples in the following table were generated, using the respective commercially available building blocks.

| | | | | MS, |
|---|---|---|---|---|
| | | | Building | ESI: |
| Ex. | Structure | Systematic Name | Blocks | m/z |
| B.198 | 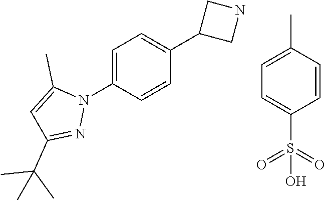 | 1-[4-(Azetidin-3- yl)phenyl]-3-tert-butyl- 5-methyl-pyrazole;4- methylbenzenesulfonic acid | 3-tert- Butyl-5- methyl- 1H- pyrazole (CAS RN 96440- 80-1) | 270.3 [M + H]⁺ |
| B.199 | 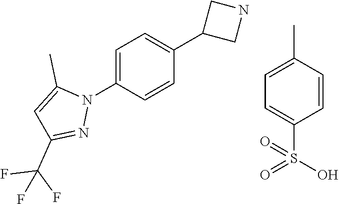 | 1-[4-(Azetidin-3- yl)phenyl]-5-methyl-3- (trifluoromethyl) pyrazole; 4- methylbenzenesulfonic acid | 5- Methyl- 3- (trifluoro methyl)- 1H- pyrazole (CAS RN: 10010- 93-2) | 282.2 [M + H]⁺ |
| B.205 | 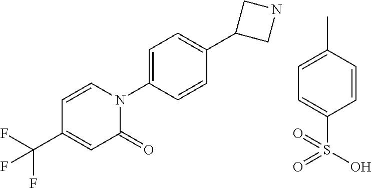 | 1-[4-(Azetidin-3- yl)phenyl]-4- (trifluoromethyl) pyridin-2-one;4- methylbenzenesulfonic acid | 4- (trifluoro methyl)- 2- pyridone (CAS RN 50650- 59-4) | 295.1 [M + H]⁺ |
| B.206 | 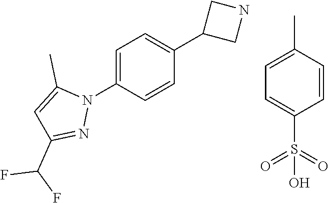 | 1-[4-(Azetidin-3- yl)phenyl]-3- (difluoromethyl)-5- methyl-pyrazole;4- methylbenzenesulfonic acid | 3- (Difluoro methyl)- 5- methyl- 1H- pyrazole (CAS RN 936033- 61-3) | 264.2 [M + H]⁺ |
| B.208 | 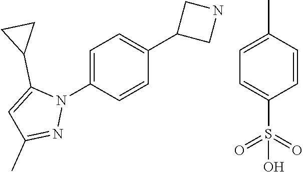 | 1-[4-(Azetidin-3- yl)phenyl]-5- cyclopropyl-3-methyl- pyrazole;4- methylbenzenesulfonic acid | 3- cyclopropyl- 5- methyl- 1H- pyrazole (CAS RN 1287752- 76-4) | 254.2 [M + H]⁺ |
| B.209 | 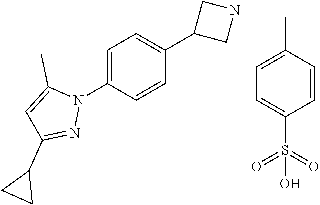 | 1-[4-(Azetidin-3- yl)phenyl]-3- cyclopropyl-5-methyl- pyrazole;4- methylbenzenesulfonic acid | 3- cyclopropyl- 5-methyl- 1H- pyrazole (CAS RN 1287752- 76-4) | 254,2 [M + H]⁺ |
| B.215 | 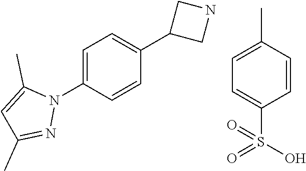 | 1-[4-(Azetidin-3- yl)phenyl]-3,5- dimethyl-pyrazole;4- methylbenzenesulfonic acid | 1-(4- Bromophenyl)- 3,5- dimethyl- pyrazole (CAS RN 62546- 27-4) | 228.1 [M + H]⁺ |

### Example B.200

### (4R) or (4S)-1-[4-(Azetidin-3-yl)phenyl]-4-(trifluoromethyl)piperidin-2-one; 4-methylbenzenesulfonic Acid

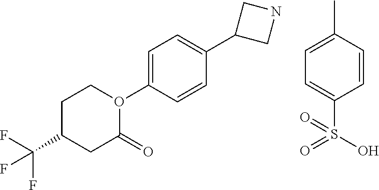

To a solution of 3-[4-[(4R)-2-keto-4-(trifluoromethyl)piperidino]phenyl]azetidine-1-carboxylic acid tert-butyl ester (59.8 mg, 0.150 mmol) in Ethyl acetate (0.871 mL) was added 4-methylbenzenesulfonic acid hydrate (8.56 mg, 0.045 mmol) and the mixture was stirred at reflux for 5 h. The reaction was allowed to cool down to 23° C. and filtered, to give the title compound (0.068 g; 96.7%) as an off-white solid. MS (ESI): m/z=299.2 [M+H]⁺

### Step a) 1-(4-Bromophenyl)-4-(trifluoromethyl)piperidin-2-one

In an argon flushed flask, 4-(trifluoromethyl)-2-piperidone (CAS RN: 1803588-50-2; 200 mg, 1.2 mmol), 1-bromo-4-iodobenzene (CAS RN: 589-87-7; 406.25 mg, 1.44 mmol), copper (I) iodide (11.4 mg, 0.060 mmol) and tripotassium phosphate (762.05 mg, 3.59 mmol) were combined with degassed DMF (2.39 mL) under Ar, to give a yellow suspension. The reaction mixture was stirred and heated to 110° C. for 48 h. The reaction mixture was poured into saturated aqueous NaHCO₃ (10 mL) and extracted with EtOAc (3×10 mL). The organic layers were combined and washed with H₂O (10 mL) and brine (20 mL), dried over Na₂SO₄, and evaporated. Purification by FC (SiO₂; heptane/EtOAc) gave the title compound (0.159 g, 42.0%) as white needles. MS (ESI): m/z=324.1 [M+H]

### Step b) Tert-Butyl 3-[4-[2-oxo-4-(trifluoromethyl)-1 piperidyl]phenyl]azetidine-1-carboxylate

To a 20 mL vial equipped with a stir bar was added photocatalyst (IR[dF(CF₃)PPY]₂(DTBPY))PF₆ (7.01 mg, 0.006 mmol), 1-(4-bromophenyl)-4-(trifluoromethyl)-2-piperidone (201.3 mg, 0.625 mmol), tert-butyl 3-bromoazetidine-1-carboxylate (153.38 μL, 0.937 mmol), TRIS(TRIMETHYLSILYL)SILANE (192.8 μL, 0.625 mmol) and anhydrous sodium carbonate (132.47 mg, 1.25 mmol). The vial was sealed and placed under Ar before DME (5.31 mL) was added. To a separate vial was added NICKEL(II) CHLORIDE ETHYLENE GLYCOL DIMETHYL ETHER COMPLEX (1.37 mg, 0.006 mmol) and 4,4′-DI-TERT-BUTYL-2,2′-BIPYRIDINE (1.68 mg, 0.006 mmol). The precatalyst vial was sealed, purged with argon, treated with DME (2.13 mL), and sonicated for 5 min, before being added to the main reaction mixture. The mixture was stirred and irradiated with a 420 nm lamp for 16 h (75% intensity) for 24 h, before being filtered and evaporated. Purification by FC (SiO₂; heptane/EtOAc) gave the title compound (0.151 g; 60.6%) as an off-white solid. MS (ESI): m/z=343.2 [M+H]

### Step c) Tert-Butyl 3-[4-[(4R) or (4S)-2-oxo-4-(trifluoromethyl)-1-piperidyl]phenyl]azetidine-1-carboxylate

tert-Butyl 3-[4-[2-oxo-4-(trifluoromethyl)-1-piperidyl]phenyl]azetidine-1-carboxylate (150 mg, 0.376 mmol) was separated by chiral SFC, to give the title compound (4R or 4S) (60.8 mg, 40.5%) as an off-white gum. MS (ESI): m/z=343.2 [M-tBu+H]⁺

### Example B.201

### (4S) or (4R)-1-[4-(Azetidin-3-yl)phenyl]-4-(trifluoromethyl)piperidin-2-one; 4-methylbenzenesulfonic Acid

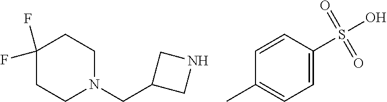

To a solution of 3-[4-[(4S)-2-keto-4-(trifluoromethyl)piperidino]phenyl]azetidine-1-carboxylic acid tert-butyl ester (62.4 mg, 0.157 mmol) in EtOAc (0.909 mL) was added 4-methylbenzenesulfonic acid hydrate (31.28 mg, 0.164 mmol) and the mixture was stirred at reflux for 5 h. The reaction was allowed to cool down, and the resulting precipitate was filtered off, to give the title compound (0.064 g; 87.8%) as a white solid. MS (ESI): m/z=299.2 [M+H]⁺

### Step a) 1-(4-Bromophenyl)-4-(trifluoromethyl)piperidin-2-one

In an argon flushed flask, 4-(trifluoromethyl)-2-piperidone (CAS RN: 1803588-50-2; 200 mg, 1.2 mmol), 1-bromo-4-iodobenzene (CAS RN: 589-87-7; 406.25 mg, 1.44 mmol), copper (I) iodide (11.4 mg, 0.060 mmol) and tripotassium phosphate (762.05 mg, 3.59 mmol) were combined with degassed DMF (2.39 mL) under Ar, to give a yellow suspension. The reaction mixture was stirred and heated to 110° C. for 48 h. The reaction mixture was poured into saturated aqueous NaHCO₃ (10 mL) and extracted with EtOAc (3×10 mL). The organic layers were combined and washed with H₂O (10 mL) and brine (20 mL), dried over Na₂SO₄, and evaporated. Purification by FC (SiO₂; heptane/EtOAc) gave the title compound (0.159 g, 42.0%) as white needles. MS (ESI): m/z=324.1 [M+H]⁺

### Step b) Tert-Butyl 3-[4-[2-oxo-4-(trifluoromethyl)-1 piperidyl]phenyl]azetidine-1-carboxylate

To a 20 mL vial equipped with a stir bar was added photocatalyst (IR[dF(CF₃)PPY]₂(DTBPY))PF₆ (7.01 mg, 0.006 mmol), 1-(4-bromophenyl)-4-(trifluoromethyl)-2-piperidone (201.3 mg, 0.625 mmol), tert-butyl 3-bromoazetidine-1-carboxylate (153.38 μL, 0.937 mmol), TRIS(TRIMETHYLSILYL)SILANE (192.8 μL, 0.625 mmol) and anhydrous sodium carbonate (132.47 mg, 1.25 mmol). The vial was sealed and placed under Ar before DME (5.31 mL) was added. To a separate vial was added NICKEL(II) CHLORIDE ETHYLENE GLYCOL DIMETHYL ETHER COMPLEX (1.37 mg, 0.006 mmol) and 4,4′-DI-TERT-BUTYL-2,2′-BIPYRIDINE (1.68 mg, 0.006 mmol). The precatalyst vial was sealed, purged with argon, treated with DME (2.13 mL), and sonicated for 5 min, before being added to the main reaction mixture. The mixture was stirred and irradiated with a 420 nm lamp for 16 h (75% intensity) for 24 h, before being filtered and evaporated. Purification by FC (SiO₂; heptane/EtOAc) gave the title compound (0.151 g; 60.6%) as an off-white solid. MS (ESI): m/z=343.2 [M+H]⁺

### Step c) Tert-Butyl 3-[4-[(4S) or (4R)-2-oxo-4-(trifluoromethyl)-1-piperidyl]phenyl]azetidine-1-carboxylate

tert-Butyl 3-[4-[2-oxo-4-(trifluoromethyl)-1-piperidyl]phenyl]azetidine-1-carboxylate (150 mg, 0.376 mmol) was separated by chiral SFC, to give the title compound (4R or 4S) (63.4 mg, 42.3%) as a white waxy solid. MS (ESI): m/z=343.2 [M-tBu+H]⁺.

### Example B.202

### 7-(5-Chloro-3-fluoro-2-pyridyl)-2,7-diazaspiro[3.5]nonane; 4-methylbenzenesulfonic Acid

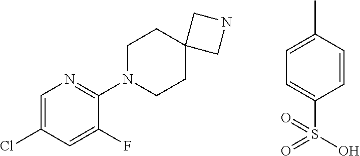 [SMILES: Fc1cc(Cl)cnc1N1CCC2(CC1)CNC2 ; Cc1ccc(S(=O)(=O)O)cc1]

To a solution of 7-(5-chloro-3-fluoro-2-pyridyl)-2,7-diazaspiro[3.5]nonane-2-carboxylic acid tert-butyl ester (112 mg, 0.315 mmol) in EtOAc (1.83 mL) was added 4-methylbenzenesulfonic acid hydrate (122.74 mg, 0.645 mmol). The mixture was stirred at reflux for 2 h, before being allowed to cool down and evaporated, to give the title compound (0.198 g; 99.8%) as a pink foam. MS (ESI): m/z=256.2 [M+H]⁺

### Step a) Tert-Butyl 7-(S-chloro-3 fluoro-2 pyridyl)-2,7-diazaspiro[3.5]nonane-2-carboxylate

To a suspension of 5-chloro-2,3-difluoro-pyridine (CAS RN: 89402-43-7; 50 mg, 0.334 mmol) and 2,7-diazaspiro[3.5]nonane-2-carboxylic acid tert-butyl ester (CAS RN: 236406-55-6; 79.44 mg, 0.351 mmol) in DMSO (1.39 mL) under Ar were added Et₃N (51.27 μL, 0.368 mmol) and the mixture was heated at 80° C. for 72 h. The reaction was cooled down, diluted with EtOAc (5 mL), and washed with water (3×5 mL) and brine (5 mL). The organic layer was dried over Na₂SO₄, filtered and evaporated, to give the title compound (0.113 g, 95.0%) as an off-white waxy solid. MS (ESI): m/z=300.2 [M+H-Buten]⁺

### Example B.204

### 7-(4-Chloro-2-fluoro-phenyl)-2,7-diazaspiro[3.5]nonane; 4-methylbenzenesulfonic Acid

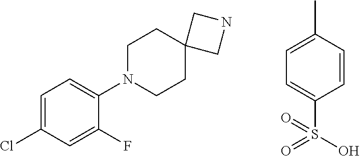 [SMILES: Fc1cc(Cl)ccc1N1CCC2(CC1)CNC2 ; Cc1ccc(S(=O)(=O)O)cc1]

A solution of 7-(4-chloro-2-fluoro-phenyl)-2,7-diazaspiro[3.5]nonane-2-carboxylic acid tert-butyl ester (100 mg, 0.282 mmol) in EtOAc (1.64 mL) was treated with 4-methylbenzenesulfonic acid hydrate (109.89 mg, 0.578 mmol) and stirred at reflux for 2 h. The resulting precipitate was filtered off, to give the title compound as a pink solid. MS (ESI): m/z=255.2 [M+H]⁺

### Step a) Tert-Butyl 7-(4-chloro-2 fluoro phenyl)-2,7-diazaspiro[3.5]nonane-2-carboxylate

Under argon, to a 2 mL microwave vial was added 1-bromo-4-chloro-2-fluoro-benzene (125.15 μL, 1. mmol, CASRN 1996-29-8), 2,7-diazaspiro[3.5]nonane-2-carboxylic acid tert-butyl ester (453.85 mg, 2.01 mmol, CAS RN 236406-55-6), palladium (H) acetate (22.51 mg, 0.100 mmol), 2-(di-t-butylphosphino)biphenyl (29.92 mg, 0.100 mmol, CAS RN 224311-51-7), sodium tert-butoxide (144.54 mg, 1.5 mmol) and toluene (2.1 mL). The reaction was then stirred and irradiated with microwave irradiation at 130° C. for 30 min. The reaction was then diluted with EtOAc (10 mL) and filtered, then washed with water (3×10 mL) and brine (15 mL). The organic layer was then dried over Na₂SO₄ and concentrated in vacuo. Purification by FC (SiO₂; heptane/EtOAc) gave the title compound (0.273 g, 72.9%) as a colorless waxy solid. MS (ESI): m/z=355.3 [M+H]⁺

### Example B.207

### 2-(Azetidin-3-yl)-5-spiro[3.3]heptan-2-yl-pyrazine; 4-methylbenzenesulfonic Acid

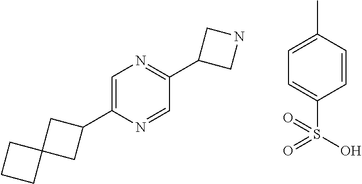 [SMILES: c1nc(C2CC3(CCC3)C2)cnc1C1CNC1 ; Cc1ccc(S(=O)(=O)O)cc1]

A solution of 3-(5-spiro[3.3]heptan-2-ylpyrazin-2-yl)azetidine-1-carboxylic acid tert-butyl ester (66 mg, 0.112 mmol) and p-toluenesulfonic acid monohydrate (70.42 mg, 0.370 mmol) in ethyl acetate (0.500 mL) was stirred at reflux for 60 min. The resulting suspension was filtered, and the filtrate was evaporated, to give the title compound (0.075 g, 71.7%) as a light brown foam. MS (ESI): m/z=230.2 [M+H]+

### Step a) Tert-Butyl 3-(5-bromopyrazin-2 yl)azetidine-1-carboxylate

To a suspension of zinc dust (346.4 mg, 5.3 mmol) in THE (8 mL) were added 1,2-dibromoethane (30.44 uL, 0.353 mmol) and chlorotrimethylsilane (44.93 μL, 0.353 mmol) and the suspension was stirred at 60° C. for 15 min. A solution of tert-butyl 3-iodoazetidine-1-carboxylate (613.5 μL, 3.53 mmol, CAS RN 254454-54-1) in N,N-DMA (8 mL) was added. The reaction mixture was stirred an additional 15 min at 60° C. before cooling to RT. 2,5-Dibromopyrazine (882.25 mg, 3.71 mmol, CAS RN 23229-26-7), 1, P-bis(diphenylphosphino)ferrocene-palladium(II)dichloride DCM complex (144.23 mg, 0.177 mmol) and Cu(I)iodide (34.32 mg, 0.177 mmol) were added and stirring was continued at 80° C. for 3 h. The reaction mixture was diluted with ethyl acetate and water and the mixture was filtered. The filtrate layers were separated. The aqueous layer was extracted twice with ethyl acetate. The organic layers were washed twice with water, dried over MgSO₄, filtered, treated with silica gel and evaporated. Purification by FC (SiO₂; heptane/EtOAc) gave the title compound (0.156 g; 12.9%) as a colorless solid. MS (ESI): m/z=258.0 [M-C4H8+H]⁺

### Step b) Tert-Butyl 3-(5-spiro[3.3]heptan-2-ylpyrazin-2-yl)azetidine-1-carboxylate

In a sealed and argon filled tube were combined 2-bromospiro[3.3]heptane (100.3 mg, 0.573 mmol), 3-(5-bromopyrazin-2-yl)azetidine-1-carboxylic acid tert-butyl ester (150 mg, 0.477 mmol), dichloronickel; 1,2-dimethoxyethane (10.49 mg, 0.048 mmol), bis[3,5-difluoro-2-[5-(trifluoromethyl)-2-pyridyl]phenyl]iridium(1+); 4-tert-butyl-2-(4-tert-butyl-2-pyridyl)pyridine; hexafluorophosphate (5.36 mg, 0.005 mmol) and 4-tert-butyl-2-(4-tert-butyl-2-pyridyl)pyridine (12.81 mg, 0.048 mmol) and the mixture was suspended in ethylene glycol dimethyl ether (4 mL) before addition of bis(trimethylsilyl)silyl-trimethyl-silane (147.29 μL, 0.477 mmol) and 2,6-dimethylpyridine (111.21 μL, 0.955 mmol). After degassing with argon, the suspension was stirred in the Penn photoreactor with a 420 nm lamp (50% intensity) for 2 h. The reaction was treated with silica gel and evaporated. Purification by FC (SiO₂; heptane/EtOAc) gave the title compound (0.042 g, 21.3%) as a colorless solid. MS (ESI): m/z=330.2 [M+H]+

In analogy to Example B.207, Examples in the following table were prepared using the respective commercially available building blocks described in Step b).

| | | | | MS, |
|---|---|---|---|---|
| | | | Building | ESI: |
| Ex. | Structure | Systematic Name | Blocks | m/z |
| B.216 | 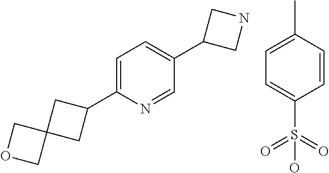 | 5-(Azetidin-3-yl)- 2-(2- oxaspiro[3.3] heptan-6- yl)pyridine;4- methylbenzenesul fonic acid | 6-bromo-2- oxaspiro[3.3] heptane (CAS RN 1363380-78-2) | 231.1 [M + H]⁺ |
| B.219 | 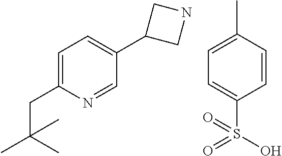 | 5-(Azetidin-3-yl)- 2-(2,2- dimethylpropyl) pyridine;4- methylbenzenesul fonic acid | 1-bromo-2,2- dimethyl- propane (CAS RN 630-17-1) | 205.1 [M + H]⁺ |
| B.220 | 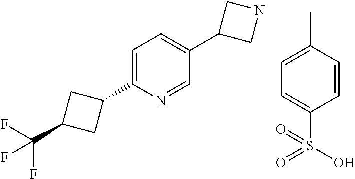 | (trans)-5- (Azetidin-3-yl)-2- [3- (trifluoromethyl) cyclobutyl]pyridine;4- methylbenzenesul fonic acid | 1-bromo-3- (trifluoromethyl) cyclobutane (CAS RN 2247103-30-4) | 257.1 [M + H]⁺ |
| B.221 | 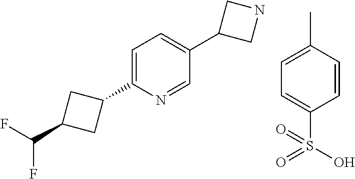 | (trans)-5- (Azetidin-3-yl)-2-[3- (difluoromethyl) cyclobutyl]pyridine;4- methylbenzenesul fonic acid | 1-bromo-3- (difluoromethyl) cyclobutane (CAS RN 2241140-79-2) | 239.1 [M + H]⁺ |

### Example B.210

### 5-[5-(Azetidin-3-yl)-2-pyridyl]-2,2-difluoro-5-azaspiro[2.4]heptane; 4-methylbenzenesulfonic Acid

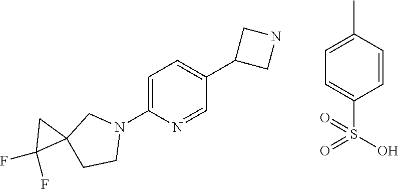 [SMILES: FC1(F)CC12CCN(c1ccc(C3CNC3)cn1)C2 ; Cc1ccc(S(=O)(=O)O)cc1]

To a solution of 3-[6-(2,2-difluoro-5-azaspiro[2.4]heptan-5-yl)-3-pyridyl]azetidine-1-carboxylic acid tert-butyl ester (100 mg, 0.274 mmol) in EtOAc (1.59 mL) was added 4-methylbenzenesulfonic acid hydrate (106.71 mg, 0.561 mmol) and the mixture was stirred at reflux for 2 h. The mixture was then evaporated, to give the title compound (0.184 g; 92.1%) as a light yellow foam. MS (ESI): m/z=266.2 [M+H]⁺

### Step a) Tert-Butyl 3-(6-bromo-3-pyridyl)azetidine-1-carboxylate

To a solution of 3-iodoazetidine-1-carboxylic acid tert-butyl ester (1 g, 3.53 mmol, CAS RN 254454-54-1) in isopropanol (10 mL) were added under argon (6-bromo-3-pyridyl)boronic acid (1.43 g, 7.06 mmol, CAS RN 223463-14-7), rac-(1 S,2S)-2-aminocyclobexanol (24.41 mg, 0.212 mmol), diiodonickel (66.23 mg, 0.212 mmol) and 2 M sodium bis(trimethylsilyl)amide in THF (3.53 mL, 7.06 mmol) and the mixture was stirred at RT for 10 min before being irridiated in the microwave oven for 30 min at 80° C. The mixture was poured on water and ethyl acetate and the layers were filtered. The filtrate layers were separated. The aqueous layer was extracted twice with ethyl acetate. The organic layers were dried over MgSO₄, filtered, treated with silica gel and evaporated. Purification by FC (SiO₂; heptane/EtOAc) gave the title compound (0.439 g; 37.7%) as a colorless semisolid. MS (ESI): m/z=315.1 [M+H]⁺

### Step b) Tert-Butyl 3-[6-(2,2-difluoro-5-azaspiro[2.4]heptan-S yl)-3 pyridyl]azetidine-1-carboxylate

To a suspension of 3-(6-bromo-3-pyridyl)azetidine-1-carboxylic acid tert-butyl ester (175 mg, 0.559 mmol) and 2,2-difluoro-5-azaspiro[2.4]heptane; hydrochloride (94.77 mg, 0.559 mmol) in 1,4-dioxane (3.5 mL) under argon were added PdCl(crotyl)AmPhos (13.78 mg, 0.028 mmol), sodium tert-butoxide (2M in THF) (838.15 μL, 1.68 mmol), and the mixture was heated in a microwave oven at 80° C. for 3 h. The mixture was filtered, then the filtrate was treated with silica gel and evaporated. Purification by FC (SiO₂; heptane/EtOAc) gave the title compound (0.107 g; 50.2%) as a yellow solid. MS (ESI): m/z=366.2 [M+H]⁺.

In analogy to Example B.210, Examples in the following table were prepared using the respective commercially available building blocks described in Step b).

| | | | | MS, |
|---|---|---|---|---|
| | | | Building | ESI: |
| Ex. | Structure | Systematic Name | Blocks | m/z |
| B.211 | 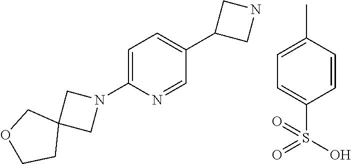 | 2-[5-(Azetidin-3- yl)-2-pyridy1]-6- oxa-2- azaspiro[3.4] octane;4- methylbenzenesul fonic acid | 6-Oxa-2- azaspiro[3.4] octane;oxalic acid (CAS RN 410070-90-5) | 246.2 [M + H]⁺ |
| B.214 | 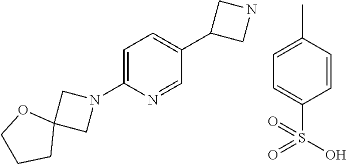 | 2-[5-(Azetidin-3- yl)-2-pyridy1]-5- oxa-2- azaspiro[3.4] octane;4- methylbenzenesul fonic acid | 5-Oxa-2- azaspiro[3.4] octane; oxalic acid (CAS RN 145309-24-6) | 246.2 [M + H]⁺ |
| B.222 | 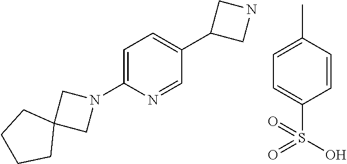 | 2-[5-(Azetidin-3- yl)-2-pyridy1]-2- azaspiro[3.4] octane;4- methylbenzenesul fonic acid | 2-azaspiro[3.4] octane (CAS RN 665-41-8) and tert-butyl 3-(6- chloro-3- pyridyl)azetidine- 1-carboxylate (CAS RN 870689-19-3) | 244.2 [M + H]⁺ |

### Example B.212

### 3-[4(Azetidin 3-yl)phenyl]-1-(trifluoromethyl)cyclobutanol; Hydrochloride

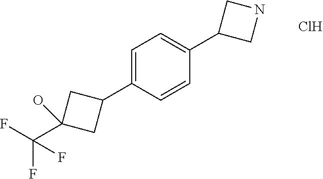 [SMILES: OC1(C(F)(F)F)CC(c2ccc(C3CNC3)cc2)C1 ; Cl]

A solution of tert-butyl 3-[4-[3-hydroxy-3-(trifluoromethyl)cyclobutyl]phenyl]azetidine-1-carboxylate (76 mg, 0.176 mmol) in chloroform (0.8 mL) was treated dropwise with 3 M HCl in CypOMe (176 μL, 0.528 mmol) and the solution was stirred at RT overnight. The reaction is a solution with an oily residue. It was completely evaporated (at 20° C.) to get the title compound (0.061 g, 90.1°/o) as a crude light yellow gum. MS (ESI): m/z=272.1 [M+H]⁺

### Step a) Tert-Butyl 3-[4-[3-hydroxy-3-(trifluoromethyl)cyclobutyl]phenyl]azetidine-1-carboxylate

To a solution of tert-butyl 3-[4-(3-oxocyclobutyl)phenyl]azetidine-1-carboxylate (97 mg, 0.283 mmol, CAS RN 2634758-48-6) in tetrahydrofuran (0.660 mL) at 0° C. was added (trifluoromethyl)trimethylsilane (67.88 μL, 0.425 mmol) followed by addition of 1 M tetrabutylammonium fluoride in THE (2.83 μL, 0.003 mmol). The solution was stirred at RT for 2 h. To the dark brown solution was added 1 M HCl in water (849.69 μL, 0.850 mmol). The mixture was vigourously stirred at RT overnight. It was diluted with ethyl acetate and water and the layers were separated. The aqueous layer was extracted once with ethyl acetate. The organic layers were dried over MgSO₄, filtered, treated with silica gel and evaporated. Purification by FC (SiO₂; heptane/EtOAc) gave the title compound as a colorless solid (0.076 g, 62.1%). MS (ESI): m/z=316.1 [M-C4H8+H]⁺

### Example B.213

### 2-(Azetidin-3-yl)-5-[3-(trifluoromethyl)azetidin-1-yl]pyridine; 4-methylbenzenesulfonic Acid

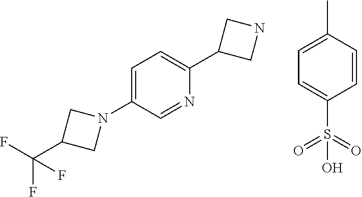

To a solution of 3-[5-[3-(trifluoromethyl)azetidin-1-yl]-2-pyridyl]azetidine-1-carboxylic acid tert-butyl ester (60 mg, 0.163 mmol) in ethyl acetate (0.400 mL) was treated with p-toluenesulfonic acid monohydrate (68.15 mg, 0.358 mmol) and the mixture was heated at reflux for 40 min. The suspension was allowed to cool down overnight while stirring. The suspension was filtered. The filter cake was washed with ethyl acetate to get the title compound (0.090 g, 87.2%) as a light brown solid. MS (ESI): m/z=258.1 [M+H]⁺.

### Step a) Tert-Butyl 3-[5-[3-(trifluoromethyl)azetidin-1 yl]-2-pyridyl]azetidine-1-carboxylate

To a suspension of tert-butyl 3-(5-bromo-2-pyridyl)azetidine-1-carboxylate (86 mg, 0.275 mmol, CAS RN 1922143-52-9) and 3-(trifluoromethyl)azetidine; hydrochloride (66.54 mg, 0.412 mmol, CAS RN 1221272-90-7) in 1,4-dioxane (1.75 mL) under argon were added RuPhos Pd G4 (11.68 mg, 0.014 mmol, CAS RN: 1599466-85-9), sodium tert-butoxide (2M in THF) (411.89 uL, 0.824 mmol), and and the mixture was heated in a microwave oven at 80° C. for 2 h. The mixture was diluted with ethyl acetate and water and the layers were separated. The aqueous layer was extracted twice with ethyl acetate. The aqueous layer was extracted once with ethyl acetate. The organic layers were dried over MgSO₄, filtered, treated with silica gel and evaporated. Purification by FC (SiO₂; heptane/EtOAc) gave the title compound (0.060 g; 59.3%) as a light brown gum. MS (ESI): m/z=358.2 [M+H]⁺

### Example B.217

### 3-(4-Cyclobutyl-3-methylsulfonyl-phenyl)azetidine; 4-methylbenzenesulfonic Acid

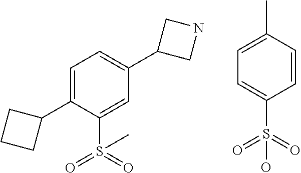

A solution of 3-(4-cyclobutyl-3-mesyl-phenyl)azetidine-1-carboxylic acid tert-butyl ester (39 mg, 0.100 mmol) and p-toluenesulfonic acid monohydrate (22.9 mg, 0.120 mmol) in ethyl acetate (0.300 mL) was heated at reflux for 1 h. A thick slurry formed. It was diluted with ethyl acetate and filtered. The filter cake was washed with ethyl acetate, to give the title compound (0.042 g, 95.7%) as a colorless solid. MS (ESI): m/z=266.2 [M+H]⁺

### Step a) Tert-Butyl 3-(4-cyclobutyl-3-fluoro-phenyl)azetidine-1-carboxylate

In a sealed and Ar filled tube were combined bromocyclobutane (CAS RN: 4399-47-7; 220.96 μL, 2.35 mmol), 3-(4-bromo-3-fluoro-phenyl)azetidine-1-carboxylic acid tert-butyl ester (CAS RN: 2222938-11-4; 327 mg, 0.782 mmol), dichloronickel; 1,2-dimethoxyethane (17.19 mg, 0.078 mmol), bis[3,5-difluoro-2-[5-(trifluoromethyl)-2-pyridyl]phenyl]iridium(1+); 4-tert-butyl-2-(4-tert-butyl-2-pyridyl)pyridine; hexafluorophosphate (8.78 mg, 0.008 mmol) and 4-tert-butyl-2-(4-tert-butyl-2-pyridyl)pyridine (21. mg, 0.078 mmol) and the mixture was suspended in ethylene glycol dimethyl ether (7 mL) before addition of bis(trimethylsilyl)silyl-trimethyl-silane (241.37 μL, 0.782 mmol) and 2,6-dimethylpyridine (182.24 μL, 1.56 mmol). After degassing with Ar the suspension was stirred in the Penn photoreactor with a 420 nm lamp (50% intensity) for 16 h, before being diluted with methanol and evaporated. Purification by FC (SiO₂; heptane/EtOAc) gave the title compound (0.080 g; 30.1%) as a colorless oil. MS (ESI): m/z=250.2 [M−C₄H₈+H]⁺

### Step b) Tert-Butyl 3-(4-cyclobutyl-3-methylsulfonyl-phenyl)azetidine-1-carboxylate

To a solution of tert-butyl 3-(4-cyclobutyl-3-fluoro-phenyl)azetidine-1-carboxylate (44 mg, 0.128 mmol) in DMSO (0.300 mL) under Ar was added NaSMe (13.48 mg, 0.192 mmol) and the mixture was stirred at 100° C. for 16 h. Another batch of NaSMe (8.99 mg, 0.128 mmol) was added and stirring continued for another 1 h at 100° C. The yellow solution was poured onto water and ethyl acetate and the layers were separated. The aqueous layer was extracted twice with ethyl acetate. The organic layers were washed twice with water, dried over MgSO₄, filtered and evaporated. The crude product (yellow oil, 50 mg) was dissolved in dichloromethane (0.300 mL) and cooled down in an ice-bath. After addition of 3-chloroperoxybenzoic acid (49.13 mg, 0.285 mmol), the suspension was stirred at 23° C. for 1 h. The suspension was diluted with DCM and half-saturated aqueous NaHCO₃ solution and the layers were separated. The aqueous layer was extracted once with DCM. The organic layers were dried over MgSO₄, filtered, and evaporated. Purification by FC (SiO₂; heptane/EtOAc) gave the title compound (0.006 g; 12.1%) as a colorless gum. MS (ESI): m/z=310.1 [M-C4H8+H]⁺

### Example B.218

### 2-[1-[5-(Azetidin-3-yl)-2-pyridyl]azetidin-3-yl]propan-2-ol; 4-methylbenzenesulfonic Acid

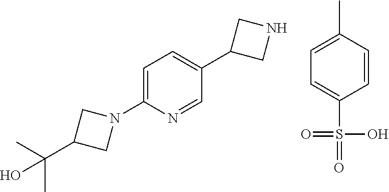

A suspension of 3-[6-[3-(1-hydroxy-1-methyl-ethyl)azetidin-1-yl]-3-pyridyl]azetidine-1-carboxylic acid tert-butyl ester (50 mg, 0.131 mmol) and p-toluenesulfonic acid monohydrate (54.8 mg, 0.288 mmol) in ethyl acetate (0.900 mL) was stirred at reflux for 1 h before being evaporated, to get the title compound (0.091 g, 93.9%) as a light brown gum. MS (ESI): m/z=248.1 [M+H]⁺

### Step a) Tert-Butyl 3-[6-[3-(1-hydroxy-1-methyl-ethyl)azetidin-1-yl]-3-pyridyl]azetidine-1-carboxylate

To a suspension of 3-(6-chloro-3-pyridyl)azetidine-1-carboxylic acid tert-butyl ester (205 mg, 0.763 mmol, CAS RN 870689-19-3) and 2-(azetidin-3-yl)propan-2-ol; hydrochloride (115.67 mg, 0.763 mmol, CAS RN 1357923-33-1) in tert.butanol (4.5 mL) under argon were added x-phos (32.73 mg, 0.069 mmol, CAS RN 564483-18-7), tris(dibenzylideneacetone)dipalladium (0) chloroform adduct (23.69 mg, 0.023 mmol, CAS RN 52522-40-4), and cesium carbonate (994.16 mg, 3.05 mmol) and the mixture was heated in a microwave oven at 90° C. for 3 h. The mixture was filtered, then the filtrate was treated with silica gel and evaporated. Purification by FC (SiO₂; heptane/EtOAc) gave the title compound (0.050 g; 17.1%) as a light yellow solid. MS (ESI): m/z=348.2 [M+H]⁺

### Example B.223

### 3-[[2-methoxy-4-(trifluoromethyl)phenyl]methoxy]azetidine; 2,2,2-trifluoroacetic Acid

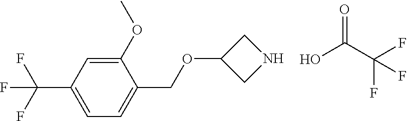 [SMILES: COc1cc(C(F)(F)F)ccc1COC1CNC1 ; O=C(O)C(F)(F)F]

To a solution of tert-butyl 3-((2-methoxy-4-(trifluoromethyl)benzyl)oxy)azetidine-1-carboxylate (400 mg, 1.11 mmol) in CH₂Cl₂ (5 mL) was added TFA (426 μL, 5.53 mmol). The mixture was stirred for 3 h at 25° C. before being evaporated, to give the title compound (475 mg, quant.) as a crude product which was directly used in the next step. MS (ESI): m/z=262.2 [M+H]⁺

### Step a) Tert-butyl 3-[[2-methoxy-4-(trifluoromethyl)phenyl]methoxy]azetidine-1-carboxylate

To a solution of tert-butyl 3-hydroxyazetidine-1-carboxylate (CAS RN: 141699-55-0; 200 mg, 1.15 mmol) in dry THF (5 mL) was added potassium tert-butoxide (1.65 M solution in THF) (735 μL, 1.21 mmol), at 25° C. The reaction mixture was stirred for 15 min at this temperature, before being treated with 1-(bromomethyl)-2-methoxy-4-(trifluoromethyl)benzene (CAS RN: 886500-59-0; 311 mg, 1.15 mmol). The mixture was stirred for another 14 h, and diluted with EtOAc and extracted with 1 M aqueous NaHCO₃. The organic phase was collected and the aqueous phase was back-extracted with EtOAc. The combined organic phases were dried over Na₂SO₄, filtered, and evaporated, to give the title compound (401 mg, 96% yield) as a crude product, which as directly used in the next step. MS (ESI): m/z=306.1 [M-tBu+H]⁺

In analogy to Example B.223, the following building blocks were generated using tert-butyl 3-hydroxyazetidine-1-carboxylate (CAS RN: 141699-55-0) and the relevant commercial building block in Step a).

| | | | | MS, |
|---|---|---|---|---|
| | | | Building | ESI: |
| Ex. | Structure | Systematic Name | Blocks | m/z |
| B.236 | 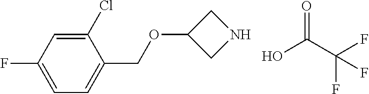 [SMILES: Fc1ccc(COC2CNC2)c(Cl)c1 ; O=C(O)C(F)(F)F] | 3-[(2-chloro-4- fluoro- phenyl)methoxy] azetidine;2,2,2- trifluoroacetic acid | 2-Chloro-4- fluorobenzy 1 bromide (CAS RN: 45767-66-6) | 216.1 [M + H]⁺ |

### Example B.224

### 3-[[2-fluoro-4-(trifluoromethoxy)phenyl]methoxy]azetidine; 4-methylbenzenesulfonic Acid

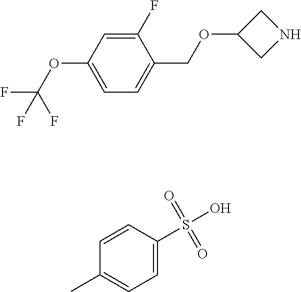 [SMILES: Fc1cc(OC(F)(F)F)ccc1COC1CNC1 ; Cc1ccc(S(=O)(=O)O)cc1]

To a solution of tert-butyl 3-((2-fluoro-4-(trifluoromethoxy)benzyl)oxy)azetidine-1-carboxylate (2.00 g, 5.47 mmol) in EtOAc (13 mL) was added 4-methylbenzenesulfonic acid monohydrate (1.09 g, 5.75 mmol), at 25° C. The mixture was then heated to 80° C., and stirred for 18 h at this temperature before being evaporated, to give the title compound (2.14 g, 88% yield) as a white solid. MS (ESI): m/z=266.2 [M-Tos+H]⁺

### Step a): Tert-butyl 3-[[2 fluoro-4-(trifluoromethoxy)phenyl]methoxy]azetidine-1-carboxylate

To a solution of tert-butyl 3-hydroxyazetidine-1-carboxylate (CAS RN: 141699-55-0; 1.00 g, 5.77 mmol) in dry THE (25 mL) was added potassium tert-butoxide (1.65 M solution in THF) (3.85 mL, 6.35 mmol), at 25° C. under Ar. The mixture was stirred for 30 min at this temperature, before being treated with 1-(bromomethyl)-2-fluoro-4-(trifluoromethoxy)benzene (CAS RN: 1240257-47-9; 1.58 g, 5.77 mmol). The mixture was stirred for another 18 h at this temperature, diluted with EtOAc, and extracted with 1 M NaHCO₃. The aqueous phase was back-extracted with EtOAc. The combined organic phases were dried over Na₂SO₄, filtered, and evaporated, to give the title compound (2.00 g, 93% yield) as a yellow viscous liquid. MS (ESI): m/z=310.1 [M-tBu+H]⁺

In analogy to Example B.224, the following building blocks were generated using the relevant commercial building blocks and tert-butyl 3-hydroxyazetidine-1-carboxylate (CAS RN: 141699-55-0) in Step a).

| | | | | MS, |
|---|---|---|---|---|
| | | | Building | ESI: |
| Ex. | Structure | Systematic Name | Blocks | m/z |
| B.227 | 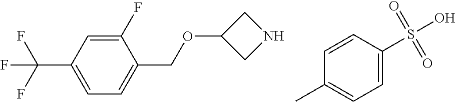 [SMILES: Fc1cc(C(F)(F)F)ccc1COC1CNC1 ; Cc1ccc(S(=O)(=O)O)cc1] | 3-[[2-fluoro-4- (trifluoromethyl)phenyl] methoxy]azetidine; 4- methylbenzenesulfonic acid | 1- (chloromethyl)- 2-fluoro-4- (trifluoromethyl) benzene (CAS RN: 1000339- 60-5) | 250.2 [M − Tos + H] ⁺ |
| B.231 | 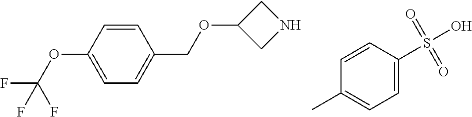 [SMILES: FC(F)(F)Oc1ccc(COC2CNC2)cc1 ; Cc1ccc(S(=O)(=O)O)cc1] | 4- methylbenzenesulfonic acid;3-[[4- (trifluoromethoxy) phenyl]methoxy] azetidine | 1- (Bromomethyl)- 4- (trifluoromethoxy) benzene (CAS RN: 50824-05- 0) | 248.2 [M − Tos + H] ⁺ |
| B.244 | 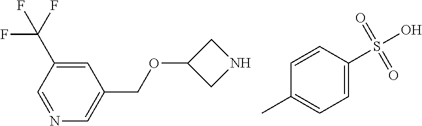 [SMILES: FC(F)(F)c1cncc(COC2CNC2)c1 ; Cc1ccc(S(=O)(=O)O)cc1] | 3-(azetidin-3- yloxymethyl)-5- (trifluoromethyl) pyridine;4- methylbenzenesulfonic acid | 3- (Bromomethyl)- 5- (trifluoromethyl) pyridine (CAS RN: 1227574- 31-3) | 233.4 [M − Tos + H] ⁺ |
| B.247 | 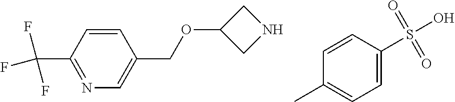 [SMILES: FC(F)(F)c1ccc(COC2CNC2)cn1 ; Cc1ccc(S(=O)(=O)O)cc1] | 5-(azetidin-3- yloxymethyl)-2- (trifluoromethyl) pyridine;4- methylbenzenesulfonic acid | 5- (bromomethyl)- 2- (trifluoromethyl) pyridine (CAS RN: 108274-33- 5) | 233.1 [M − Tos + H] ⁺ |

### Example B.225

### 3-[[2-fluoro-4-(trifluoromethylsulfonyl)phenyl]methoxy]azetidine; 4-methylbenzenesulfonic Acid

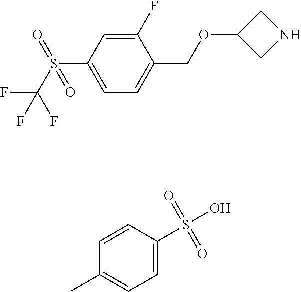 [SMILES: O=S(=O)(c1ccc(COC2CNC2)c(F)c1)C(F)(F)F ; Cc1ccc(S(=O)(=O)O)cc1]

A solution of p-toluenesulfonic acid (674.84 mg, 3.92 mmol) and tert-butyl 3-[[2-fluoro-4-(trifluoromethylsulfonyl)phenyl]methoxy]azetidine-1-carboxylate (1.35 g, 3.27 mmol) in EtOAc (14 mL) was stirred at 80° C. for 12 h. The mixture was filtered and the cake was dried, to give the title compound (915 mg, 56% yield) as a white solid. MS (ESI): m/z=314.1 [M-TsOH+H]⁺

### Step a): Tert-butyl 3-[(2-fluoro-4-iodo-phenyl)methoxy]azetidine-1-carboxylase

To a solution of tert-butyl 3-hydroxyazetidine-1-carboxylate (CAS RN: 141699-55-0; 2.92 g, 16.83 mmol) in THE (50 mL) were added potassium tert-butoxide (3.78 g, 33.66 mmol) and 1-(bromomethyl)-2-fluoro-4-iodo-benzene (CAS RN: 85510-81-2; 5.3 g, 16.83 mmol), at 25° C. under Ar. The mixture was stirred for 12 h at 30° C., before being evaporated. Purification by FC (SiO₂; PE/EtOAc) and RP-HPLC gave the title compound (3.0 g, 44% yield) as a colorless oil. 1H NMR (400 MHz, CDCl₃) δ=7.51 (dd, J=1.4, 8.1 Hz, 1H), 7.43 (dd, J=1.6, 9.1 Hz, 1H), 7.14 (t, 0.1=7.8 Hz, 1H), 4.46 (s, 2H), 4.32 (tdd, J=2.0, 4.3, 6.4 Hz, 1H), 4.11-4.05 (m, 2H), 3.89-3.83 (m, 2H), 1.44 ppm (s, 9H).

### Step b): Tert-Butyl 3-[[2 fluoro-4-(trifluoromethylsulfanyl)phenyl]methoxy]azetidine-1-carboxylate

Two batches were set up in parallel. To a 40 mL vial equipped with a magnetic stir bar were added trifluoromethylsulfanylsilver (769.63 mg, 3.68 mmol), tert-butyl 3-[(2-fluoro-4-iodo-phenyl)methoxy]azetidine-1-carboxylate (1.0 g, 2.46 mmol) and bpy (383.53 mg, 2.46 mmol) in ACN (10 mL), and then was added CuI (467.68 mg, 2.46 mmol) under N₂ atmosphere. The mixture stirred at 100° C. for 17 h under N₂ atmosphere. The mixture was filtered and evaporated. Purification by FC (SiO₂; PE/EtOAc) gave the title compound (1.68 g, 90% yield) as a white solid. MS (ESI): m/z=282.2 [M-C₅H₈O₂+H]⁺

### Step c): Tert-Butyl 3-[[2-ffluoro-4-(trifluoromethylsulfonyl)phenyl]methoxy]azetidine-1-carboxylate

To a solution of tert-butyl 3-[[2-fluoro-4-(trifluoromethylsulfanyl)phenyl]methoxy]azetidine-1-carboxylate (1.58 g, 4.14 mmol) in 1:1:2 1,2-dichloroethane/ACN/water (60 mL) was added sodium periodate (1.77 g, 8.29 mmol) and ruthenium(III) chloride hydrate (9.34 mg, 0.040 mmol), at 0° C. The mixture was stirred for 12 h at 30° C., before being extracted with EtOAc (3×). The combined organic layers were washed with brine (3×), dried over Na₂SO₄, filtered, and evaporated. Purification by FC (SiO₂; PE/EtOAc) gave the title compound (1.45 g, 85% yield) as a colorless oil. MS (ESI): m/z=258.2 [M-C₄H₈+H]⁺

In analogy to Example B.225, the following building blocks were generated using the relevant commercial building blocks and tert-butyl 3-hydroxyazetidine-1-carboxylate (CAS RN: 141699-55-0) in Step a).

| | | | | MS, |
|---|---|---|---|---|
| | | | Building | ESI: |
| Ex. | Structure | Systematic Name | Blocks | m/z |
| B.232 | 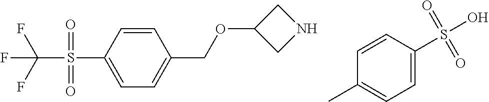 [SMILES: O=S(=O)(c1ccc(COC2CNC2)cc1)C(F)(F)F ; Cc1ccc(S(=O)(=O)O)cc1] | 4- methylbenzenesulfonic acid;3-[[4- (trifluoromethylsulfonyl) phenyl]methoxy] azetidine | 1- (Bromomethyl)- 4- iodobenzene (CAS RN: 16004-15-2) | 295.7 [M − Tos + H] ⁺ |
| B.237 | 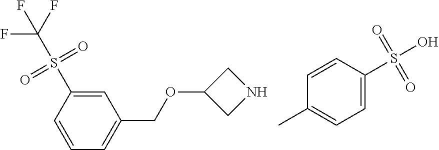 | 4- methylbenzenesulfonic acid;3-[[3- (trifluoromethylsulfonyl) phenyl]methoxy]azetidine | 3- Iodobenzyl bromide (CAS RN: 49617-83-6) | 296.3 [M − Tos + H] ⁺ |

### Example B.226

### 2-(azetidin-3-yloxy)-6-cyclopropyl-benzonitrile; 4-methylbenzenesulfonic Acid

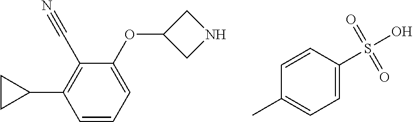 [SMILES: N#Cc1c(OC2CNC2)cccc1C1CC1 ; Cc1ccc(S(=O)(=O)O)cc1]

To a solution of tert-butyl 3-(2-cyano-3-cyclopropylphenoxy)azetidine-1-carboxylate (220 mg, 700 μmol) in EtOAc (1.67 mL) was added 4-methylbenzenesulfonic acid monohydrate (140 mg, 735 μcool). The reaction mixture was refluxed (80° C.) for 16 h, before being cooled down and evaporated, to give the title compound (268 mg, 94% yield) as a white solid. MS (ESI): m/z=215.1 [M-TsOH+H]⁺

### Step a): Tert-Butyl 3-(3-bromo-2-cyan phenoxy)azetidine-1-carboxylate

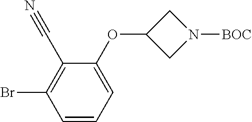 [SMILES: CC(C)(C)OC(=O)N1CC(Oc2cccc(Br)c2C#N)C1]

A solution of 2-bromo-6-hydroxybenzonitrile (CAS RN: 73289-85-7; 450 mg, 2.27 mmol) and tert-butyl 3-hydroxyazetidine-1-carboxylate (CAS RN: 141699-55-0; 394 mg, 2.27 mmol) in dry toluene (7.1 mL) was sparged with Ar, before being treated with (tributylphosphoranylidene)acetonitrile (918 μL, 3.41 mmol). The mixture was stirred at for 2 h at 100° C., before being diluted with EtOAc and washed with 1 M aqueous NaHCO₃ solution. The organic phase was collected and the aqueous phase underwent back-extraction with EtOAc. The combined organic layers were washed with brine, dried over Na₂SO₄, and evaporated. Purification by FC (SiO₂; heptane/EtOAc) gave the title compound (488 mg, 58% yield) as a light brown viscous oil. MS (ESI): m/z=299.0 [M-tBu+H]⁺

### Step b): Tert-Butyl 3-(2-cyan-3-cyclopropyl-phenoxy)azetidine-1-carboxylate

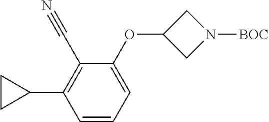 [SMILES: CC(C)(C)OC(=O)N1CC(Oc2cccc(C3CC3)c2C#N)C1]

A solution of tert-butyl 3-(3-bromo-2-cyanophenoxy)azetidine-1-carboxylate (488 mg, 1.38 mmol) in 10:1 1,4-dioxane/water (10 mL) was treated with cyclopropylboronic acid (178 mg, 2.07 mmol) and K₂CO₃ (382 mg, 2.76 mmol), at 23° C. under Ar. The mixture was sparged with Ar, before being treated with bis(triphenylphosphine)palladium (I1) chloride (97 mg, 138 μmot). The mixture was heated to 90° C. and stirred for 18 h at this temperature, before being cooled down and diluted with EtOAc. The mixture was washed with water, and the organic layer was washed with brine, dried over Na₂SO₄, filtered, and evaporated. Purification by FC (SiO₂; heptane/EtOAc) gave the title compound (220 mg, 48% yield) as a colorless viscous oil. MS (ESI): m/z=259.2 [M-tBu+H]⁺

In analogy to Example B.226, the following building blocks were generated using the relevant commercial building blocks and tert-butyl 3-hydroxyazetidine-1-carboxylate (CAS RN: 141699-55-0) in Step a).

| | | | | MS, |
|---|---|---|---|---|
| | | | Building | ESI: |
| Ex. | Structure | Systematic Name | Blocks | m/z |
| B.229 | 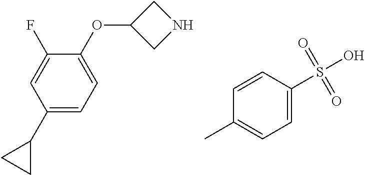 | 3-(4-cyclopropyl-2- fluoro- phenoxy)azetidine;4- methylbenzenesulfonic acid | 4-bromo-2- fluorophenol (CAS RN; 2105-94-4) | 208.1 [M − Tos + H]⁺ |
| B.233 | 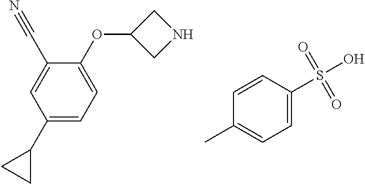 [SMILES: N#Cc1cc(C2CC2)ccc1OC1CNC1 ; Cc1ccc(S(=O)(=O)O)cc1] | 2-(azetidin-3-yloxy)-5- cyclopropyl- benzonitrile;4- methylbenzenesulfonic acid | 5-Bromo-2- hydroxybenzonitrile (CAS RN: 40530-18- 5) | 215.2 [M − Tos + H]⁺ |
| B.235 | 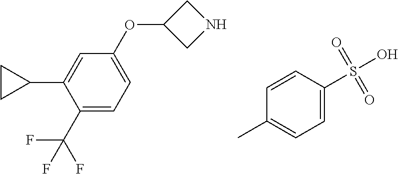 [SMILES: FC(F)(F)c1ccc(OC2CNC2)cc1C1CC1 ; Cc1ccc(S(=O)(=O)O)cc1] | 3-[3-cyclopropyl-4- (trifluoromethyl)phenoxy Jazetidine;4- methylbenzenesulfonic acid | 3-Bromo-4- (trifluoromethyl) phenol (CAS RN: 1214385- 56-4) | 258.2 [M − Tos + H]⁺ |
| B.238 | 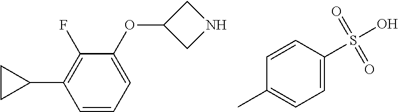 | 3-(3-cyclopropyl-2- fluoro- phenoxy)azetidine;4- methylbenzenesulfonic acid | 3-Bromo-2- fluorophenol (CAS RN: 156682-53-0) | 208.1 [M − Tos + H]⁺ |
| B.240 | 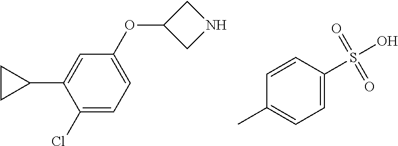 | 3-(4-chloro-3- cyclopropyl- phenoxy)azetidine;4- methylbenzenesulfonic acid | 3-Bromo-4- chlorophenol (CAS RN: 13659-24-0) | 224.1 [M − Tos + H]⁺ |
| B.243 | 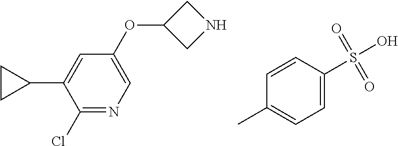 | 5-(azetidin-3-yloxy)-2- chloro-3-cyclopropyl- pyridine;4- methylbenzenesulfonic acid | 5-bromo-6- chloropyridin- 3-ol (CAS RN: 130115-85-4) | 225.1 [M − Tos + H]⁺ |

### Example B.228

### 3-(4-cyclopropylphenoxy)azetidine; 4-methylbenzenesulfonic Acid

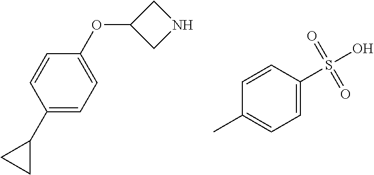

To a solution of tert-butyl 3-(4-cyclopropylphenoxy)azetidine-1-carboxylate (179 mg, 619 μmot) in EtOAc (1.47 mL) was added 4-methylbenzenesulfonic acid monohydrate (124 mg, 650 μmol), at 23° C. The reaction mixture was refluxed for 16 h at 80° C., before being cooled down and evaporated, to give the title compound (220 mg, 94% yield) as a white solid. MS (ESI): m/z=190.1 [M-TsOH+H]⁺

### Step a): Tert-Butyl 3-(4-cyclopropylphenoxy)azetidine-1-carboxylate

A solution of 4-cyclopropylphenol (CAS RN: 10292-61-2; 150 mg, 1.12 mmol) and tert-butyl 3-hydroxyazetidine-1-carboxylate (CAS RN: 141699-55-0; 194 mg, 1.12 mmol) in dry Toluene (3.49 mL) was sparged with Ar, at 23° C. (Tributylphosphoranylidene)acetonitrile (452 μL, 1.68 mmol) was added, and the mixture was stirred for 2 h at 100° C., before being cooled down, diluted with EtOAc, and washed with 1 M aqueous NaHCO₃. The organic phase was collected and the aqueous phase underwent back-extraction with EtOAc. The combined organic layers were washed with brine, dried over Na₂SO₄, filtered, and evaporated. Purification by FC (SiO₂; heptane/EtOAc) gave the title compound (179 mg, 53% yield) as a colorless viscous oil. MS (ESI): m/z=234.2 [M-tBu+H]⁺

In analogy to Example B.228, the following building blocks were generated using the relevant commercial building blocks and tert-butyl 3-hydroxyazetidine-1-carboxylate (CAS RN: 141699-55-0) in Step a).

| | | | | MS, |
|---|---|---|---|---|
| | | | Building | ESI: |
| Ex. | Structure | Systematic Name | Blocks | m/z |
| B.239 | 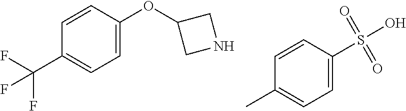 | 4- methylbenzenesulfonic acid;3-[4- (trifluoromethyl) phenoxy]azetidine | 4- (Trifluoromethyl) phenol (CAS RN: 402-45-9) | 218.1 [M − Tos + H]⁺ |

### Example B.234

### Methyl 2-[3-(azetidin-3-yloxy)phenyl]-2-methyl-propanoate; 4-methylbenzenesulfonic Acid

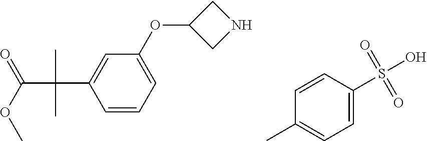 [SMILES: COC(=O)C(C)(C)c1cccc(OC2CNC2)c1 ; Cc1ccc(S(=O)(=O)O)cc1]

A solution of tert-butyl 3-[3-(2-methoxy-1,1-dimethyl-2-oxo-ethyl)phenoxy]azetidine-1-carboxylate (2.0 g, 5.72 mmol) and p-toluenesulfonic acid (1182.76 mg, 6.87 mmol) in EtOAc (25 mL) was stirred at 80° C. for 12 h. The mixture was filtered, and the crystalline solid was washed with EtOAc (10 mL) and dried, to give the title compound (2327 mg, 95.6% yield) as a grey solid. MS (ESI): m/z=250.4 [M-TsOH+H]⁺

### Step a): Tert-Butyl 3-[3-(2-methoxy-1,1-dimethyl-2-oxo-ethyl)phenoxy]azetidine-1-carboxylate

To a mixture of methyl 2-(3-bromophenyl)-2-methyl-propanoate (CAS RN: 251458-15-8; 5.0 g, 19.45 mmol), tert-butyl 3-hydroxyazetidine-1-carboxylate (CAS RN: 141699-55-0; 3.4 g, 19.45 mmol) and K₃PO₄ (8.25 g, 38.89 mmol) in toluene (100 mL) was added tBuXphos-G3-Pd (772.37 mg, 0.970 mmol), at 23° C. under N₂. The mixture was stinted at 110° C. for 12 h, before being cooled down, filtered, and evaporated. Purification by FC (SiO₂; PE/EtOAc) gave the title compound (4.10 g, 60.34% yield) as a light yellow oil. MS (ESI): m/z=250.5 [M-Boc+H]⁺

### Example B.241

### 3-[(4,4-difluorocyclohexyl)methoxy]azetidine; 4-methylbenzenesulfonic Acid

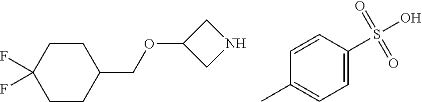

To a solution of 3-[(4,4-difluorocyclohexyl)methoxy]azetidine-1-carboxylic acid tert-butyl ester (250 mg, 0.737 mmol) in isopropyl acetate (12 mL) was added p-toluenesulfonic acid monohydrate (154.17 mg, 0.811 mmol). The mixture was stirred at 80° C. for 3 h, before being cooled down and evaporated. Trituration with Et₂O gave the title compound (264 mg, 85.43%) as a white solid. MS (ESI): m/z=206.1 [M+H]⁺

### Step a): Tert-Butyl 3-[(4,4-difluorocyclohexyl)methoxy]azetidine-1-carboxylate

To a solution of 4-(bromomethyl)-1,1-difluoro-cyclohexane (CAS RN: 858121-94-5; 1.75 g, 8.21 mmol) and 3-hydroxyazetidine-1-carboxylic acid tert-butyl ester (CAS RN: 141699-55-0; 1.49 g, 8.62 mmol) in THE (12 mL) were added tetrabutylammonium chloride (114.14 mg, 0.411 mmol) and 10 M NaOH (2.35 mL, 24.64 mmol). The mixture was stirred at 50° C. for 6 h. Citric acid 10% was added. The reaction mixture was poured into EtOAc. The organic layer was washed with water and brine, dried over Na₂SO₄, filtered, and evaporated. Purification by FC (SiO₂; heptane/EtOAc) gave the title compound (229 mg, 8.22%) as a colorless oil. MS (ESI): m/z=250.1 [M+H-Buten]⁺

In analogy to Example B.241, the following building blocks were generated using the relevant commercial building blocks and tert-butyl 3-hydroxyazetidine-1-carboxylate (CAS RN: 141699-55-0) in Step a).

| | | | | MS, |
|---|---|---|---|---|
| | | | Building | ESI: |
| Ex. | Structure | Systematic Name | Blocks | m/z |
| B.242 | 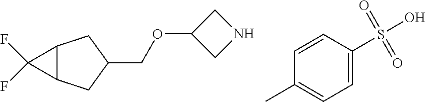 | 3-[(6,6-difluoro-3- bicyclo[3.1.0]hexanyl) methoxy]azetidine;4- methylbenzenesulfonic acid | 3- (bromomethyl) -6,6-difluoro- bicyclo[3.1.0] hexane (CAS RN: 1393569- 74-8) | 204.0 [M + H]⁺ |
| B.248 | 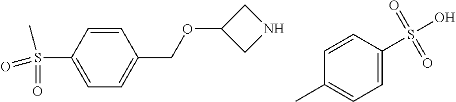 [SMILES: CS(=O)(=O)c1ccc(COC2CNC2)cc1 ; Cc1ccc(S(=O)(=O)O)cc1] | 3-[(4- methylsulfonylphenyl) methoxy]azetidine; 4- methylbenzenesulfonic acid | 1- (chloromethyl) -4-mesyl- benzene (CAS RN: 51419-59- 1) | 242.1 [M + H]⁺ |
| B.249 | 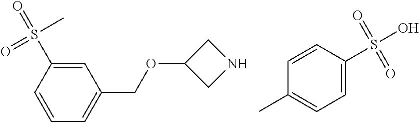 [SMILES: CS(=O)(=O)c1cccc(COC2CNC2)c1 ; Cc1ccc(S(=O)(=O)O)cc1] | 4- methylbenzenesulfonic acid;3-[(3- methylsulfonylphenyl) methoxy]azetidine | (bromomethyl)- 3-mesyl- benzene (CAS RN: 82657-76- 9) | 242.0 [M + H]⁺ |
| B.250 | 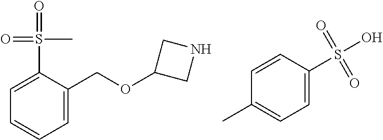 [SMILES: CS(=O)(=O)c1ccccc1COC1CNC1 ; Cc1ccc(S(=O)(=O)O)cc1] | 4- methylbenzenesulfonic acid;3-[(2- methylsulfonylphenyl) methoxy]azetidine | 1- (bromomethyl)- 2-mesyl- benzene (CAS RN: 82657-74- 7) | 242.1 [M + H]⁺ |

### Example B.245

### 3-[(2-fluoro-4-methylsulfonyl-phenyl)methoxy]azetidine; 4-methylbenzenesulfonic Acid

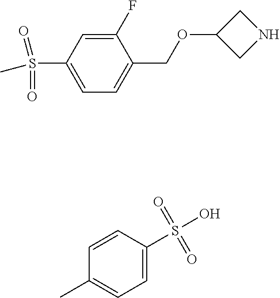 [SMILES: CS(=O)(=O)c1ccc(COC2CNC2)c(F)c1 ; Cc1ccc(S(=O)(=O)O)cc1]

PTSA (1.08 g, 5.66 mmol) was dissolved in isopropyl acetate (10 mL) and heated up to 80° C. A solution of 3-(2-fluoro-4-mesyl-benzyl)oxyazetidine-1-carboxylic acid tert-butyl ester (1.85 g, 5.15 mmol) in isopropyl acetate (10 mL) was added to the reaction mixture at 80° C. The mixture was stirred for 1.5 h at this temperature, before being cooled down to 0° C. and filtered over sintered glass, washed (2× isopropyl acetate) and dried, to give the title compound (1.76 g, 71.32%) as a white solid. MS (ESI): m/z=260.2 [M+H]⁺

### Step a): 1-(chloromethyl)-2 fluoro-4-methylsulfonyl-benzene

A solution of SOCl₂ (1.64 g, 1.01 mL, 13.81 mmol) and tetrabutylammonium chloride (72.13 mg, 0.260 mmol) in DCM (10 mL) was heated to 45° C. and stirred for 5 min at this temperature, before being treated dropwise with (2-fluoro-4-mesyl-phenyl)methanol (CAS RN: 1461702-87-3; 1.06 g, 5.19 mmol). The mixture was stirred for another 2.5 h at 45° C., before being cooled to 0° C. and quenched with 10 mL of water. The resulting solution was stirred for 10 min at 23° C., and poured into a saturated aqueous solution of NaHCO₃ (strong gas evolution). The mixture was poured into DCM, and the organic layer was washed with water and brine, dried over Na₂SO₄, filtered, and evaporated, to give the title compound (1.14 g, 88.78%) as a crude yellow solid, which was directly used in the next step.

### Step b): Tert-Butyl 3-[(2-fluoro-4-methylsulfonyl-phenyl)methoxy]azetidine-1-carboxylate

To a solution of 3-hydroxyazetidine-1-carboxylic acid tert-butyl ester (886.82 mg, 5.12 mmol) and tetrabutylammonium chloride (71.15 mg, 0.256 mmol) in THF (5 mL) was added.sodium hydroxide (2.19 g, 1.67 mL, 15.36 mmol), at 23° C. The mixture was heated up to 60° C. and a solution of 1-(chloromethyl)-2-fluoro-4-mesyl-benzene (1.14 g, 5.12 mmol) in THF (5 mL) was added. The mixture was stirred for 1.5 h at 60° C., before being cooled down and treated with citric acid 20% (pH 8). The mixture was poured into EtOAc and washed with water and brine. The organic layer was dried over Na₂SO₄, filtered and evaporated, to give the title compound (1.85 g, 90.48%) as a yellow viscous oil. MS (ESI): m/z=304.2 [M+H]⁺

### Example B.251

### 3-[4-(4-chloro-2-methylsulfonyl-phenyl)phenyl]azetidine; 4-methylbenzenesulfonic Acid

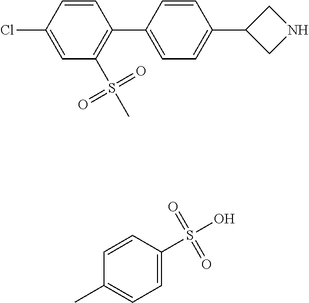 [SMILES: CS(=O)(=O)c1cc(Cl)ccc1-c1ccc(C2CNC2)cc1 ; Cc1ccc(S(=O)(=O)O)cc1]

A solution of tert-butyl 3-[4-(4-chloro-2-methylsulfonyl-phenyl)phenyl]azetidine-1-carboxylate (100.0 g, 237 mmol) and PTSA (44.89 g, 260.7 mmol) in EtOAc (1.7 L) was stirred at 80° C. for 12 h, before being filtered. The cake was washed with EtOAc (1 L) and dried under vacuum, to give the title compound (54 g, 70.8% yield) as a white solid. MS (ESI): m/z=322.1 [M-TsOH+H]⁺

### Step a): Tert-Butyl 3-(4-bromophenyl)azetidine-1-carboxylate

To a mixture of 4-bromophenylboronic acid (CAS RN: 5467-74-3; 200.04 g, 996.08 mmol) and tert-butyl 3-iodoazetidine-1-carboxylate (CAS RN: 254454-54-1; 141.0 g, 498.04 mmol) in 2-propanol (500 mL) was added rac-(1R,2R)-2-aminocyclohexan-1-ol (3.44 g, 29.88 mmol), and nickel(B) iodide (9.34 g, 29.88 mmol). A mixture of sodium bis(trimethylsilyl)amide in THE (1 L, 1000 mmol) was added slowly to the reaction mixture, under N₂ and keeping the temperature below 30° C. After the resulting mixture was stirred at 25° C. for 30 min, the mixture was heated to 80° C. and stirred for 12 h. The reaction mixture was poured onto H₂O (3 L) and EtOAc(3 L), and the layers were separated. The aqueous layer was extracted twice with EtOAc (2×2 L). The combined organic layers were evaporated, and purified by FC (SiO₂; PE/EtOAc), to give the title compound (140 g, 90.04% yield) as an off-white oil. MS (ESI): m/z=256.1 [M-tBu+H]⁺

### Step b): Tert-Butyl 3-[4-(4-chloro-2 fluoro phenyl)phenyl]azetidine-1-carboxylate

To a solution of tert-butyl 3-(4-bromophenyl)azetidine-1-carboxylate (80.0 g, 256.25 mmol), 4-chloro-2-fluorophenylboronic acid (89.36 g, 512.49 mmol) and Na₂CO₃ (54.32 g, 512.49 mmol) in 1,4-Dioxane (1.6 L mL) and water (160 ML) was added Pd(PPh₃)₂Cl₂ (9.37 g, 12.81 mmol). The mixture was then stirred for 12 h at 100° C. under N₂, before being filtered. The filtrate was evaporated, and the residue was treated with water (2 L), extracted with DCM (3×2 L). The combined organic layers were washed with brine, dried over Na₂SO₄, filtered, and evaporated. Trituration with PE (300 mL) at 25° C. gave the title compound (50 g, 53.93% yield). MS (ESI): m/z=306.1 [M-tBu+H]⁺

### Step c): Tert-Butyl 3-[4-(4-chloro-2-methylsulfanyl phenyl)phenyl]azetidine-1-carboxylate

To a solution of tert-butyl 3-[4-(4-chloro-2-fluoro-phenyl)phenyl]azetidine-1-carboxylate (150.0 g, 414.55 mmol) in DMSO (1.5 L) was added NaSMe (63.14 g, 900.82 mmol), at 0° C. The mixture was stirred for 12 h at 25° C., before being poured into water (5 L). The aqueous layer was extracted with EtOAc 83×3 L), and poured into saturated aqueous NaClO₄. The combined organic layers were washed with brine, dried over Na₂SO₄, filtered, and evaporated, to give the title compound (130 g, 80.42% yield) as a yellow oil. MS (ESI): m/z=334.1 [M−tBu+H]⁺

### Step d): Tert-Butyl 3-[4-(4-chloro-2-methylsulfonylphenyl)phenyl]azetidine-1-carboxylate

To a solution of tert-butyl 3-[4-(4-chloro-2-methylsulfanyl-phenyl)phenyl]azetidine-1-carboxylate (130.0 g, 333.38 mmol) in DCM (1.3 L) was slowly added 3-chloroperoxybenzoic acid (172.6 g, 1000 mmol), at 25° C. The mixture was stirred for 4 h at this temperature, before being washed with saturated aqueous Na₂SO₃ (3×2 L). The aqueous layer was extracted with EtOAc 3×3 L). The combined organic layers were dried over Na₂SO₄, filtered, and evaporated. Purification by FC (SiO₂; PE/EtOAc) gave the title compound (100 g, 71.1% yield) as a yellow oil. MS (ESI): m/z=365.8 [M−tBu+H]⁺

### Example B.252

### 3-[4(2-chloro-4-methylsulfonyl-phenyl)phenyl]azetidine; 4-methylbenzenesulfonic Acid

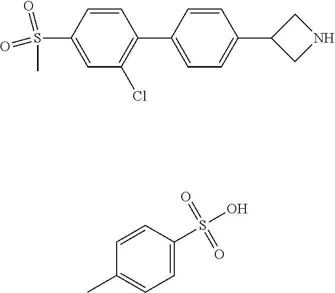 [SMILES: CS(=O)(=O)c1ccc(-c2ccc(C3CNC3)cc2)c(Cl)c1 ; Cc1ccc(S(=O)(=O)O)cc1]

To a solution of 3-[4-(2-chloro-4-mesyl-phenyl)phenyl]azetidine-1-carboxylic acid tert-butyl ester (1.03 g, 2.32 mmol) in EtOAc (6 mL) was added p-toluenesulfonic acid monohydrate (551.41 mg, 2.9 mmol) and the suspension was stirred at reflux for 1 h. After cooling down, the suspension was filtered. The filter cake was washed with a small volume of EtOAc and dried, to give the title compound (0.940 g; 82.0%) as a yellow solid. MS (ESI): m/z=322.1 [M+H]⁺.

### Step a): Tert-Butyl 3-(4-bromophenyl)azetidine-1-carboxylate

To a mixture of 4-bromophenylboronic acid (CAS RN: 5467-74-3; 200.04 g, 996.08 mmol) and tert-butyl 3-iodoazetidine-1-carboxylate (CAS RN: 254454-54-1; 141.0 g, 498.04 mmol) in 2-propanol (500 mL) was added rac-(1R,2R)-2-aminocyclohexan-1-ol (3.44 g, 29.88 mmol), and nickel(II) iodide (9.34 g, 29.88 mmol). A mixture of sodium bis(trimethylsilyl)amide in THE (1 L, 1000 mmol) was added slowly to the reaction mixture, under N₂ and keeping the temperature below 30° C. After the resulting mixture was stinted at 25° C. for 30 min, the mixture was heated to 80° C. and stirred for 12 h. The reaction mixture was poured onto H₂O (3 L) and EtOAc (3 L), and the layers were separated. The aqueous layer was extracted twice with EtOAc (2×2 L). The combined organic layers were evaporated, and purified by FC (SiO₂; PE/EtOAc), to give the title compound (140 g, 90.04% yield) as an off-white oil. MS (ESI): m/z=256.1 [M-tBu+H]⁺

### Step b): 3-[4-(2-chloro-4-mesyl-phenyl)phenyl]azetidine-1-carboxylic Acid Tert-Butyl Ester

A suspension of 2-(2-chloro-4-mesyl-phenyl)-4,4,5,5-tetramethyl-1,3,2-dioxaborolane (829.55 mg, 2.62 mmol), 3-(4-bromophenyl)azetidine-1-carboxylic acid tert-butyl ester (818 mg, 2.62 mmol), tetrakis(triphenylphosphine)palladium (0) (15.14 mg, 0.013 mmol) and K₂CO₃ (1.81 g, 13.1 mmol) in THE (10 mL) and water (1 mL) under Ar was heated for 30 min at 110° C. in a MW. The mixture was poured on water and EtOAc, and the layers were separated. The aqueous layer was extracted with EtOAC (2×). The combined organic layers were dried over MgSO₄, filtered, and evaporated. Purification by FC (SiO₂, heptane/EtOAc) gave the title compound (1.03 g; 88.5%) as a colorless oil. MS (ESI): m/z=366.1 [M-tBu+H]⁺

### Example B.253

### Methyl 2-[4-(azetidin-3-yl)phenyl]benzoate; 4-methylbenzenesulfonic Acid

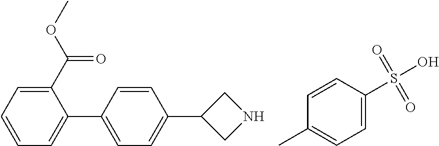

A solution of tert-butyl 3-[4-(2-methoxycarbonylphenyl)phenyl]azetidine-1-carboxylate (500.0 mg, 1.36 mmol) and p-toluenesulfonic acid monohydrate (517.69 mg, 2.72 mmol) in EtOAc (50 mL) was stirred for 3 h at 70° C., before being evaporated. Trituration with TBME/Et₂O (1:1, 50 mL) gave the title compound (524 mg, 83.23°% yield) as a white solid. MS (ESI): m/z=268.2 [M+H]⁺

### Step a): Tert-Butyl 3-(4-bromophenyl)azetidine-1-carboxylate

To a mixture of 4-bromophenylboronic acid (CAS RN: 5467-74-3; 200.04 g, 996.08 mmol) and tert-butyl 3-iodoazetidine-1-carboxylate (CAS RN: 254454-54-1; 141.0 g, 498.04 mmol) in 2-propanol (500 mL) was added rac-(1R,2R)-2-aminocyclohexan-1-ol (3.44 g, 29.88 mmol), and nickel(II) iodide (9.34 g, 29.88 mmol). A mixture of sodium bis(trimethylsilyl)amide in THE (1 L, 1000 mmol) was added slowly to the reaction mixture, under N₂ and keeping the temperature below 30° C. After the resulting mixture was stirred at 25° C. for 30 min, the mixture was heated to 80° C. and stirred for 12 h. The reaction mixture was poured onto H₂O (3 L) and EtOAc (3 L), and the layers were separated. The aqueous layer was extracted twice with EtOAc (2×2 L). The combined organic layers were evaporated, and purified by FC (SiO₂; PE/EtOAc), to give the title compound (140 g, 448.43 mmol, 90.04% yield) as an off-white oil. MS (ESI): m/z=256.1 [M-tBu+H]⁺

### Step b): Tert-Butyl 3-[4-(2-methoxycarbonylphenyl)phenyl]azetidine-1-carboxylate

A solution of tert-butyl 3-(4-bromophenyl)azetidine-1-carboxylate (2.1 g, 6.73 mmol), 2-methoxycarbonylphenylboronic acid (1.69 g, 9.42 mmol) and cesium carbonate (3.29 g, 10.09 mmol) in 1,4-Dioxane (55 mL) and Water (3 mL) was sparged with Argon for 5 min, before being treated with 1,1′-bis(diphenylphosphino)ferrocene-palladium(II)dichloride dichloromethane complex (439.1 mg, 0.540 mmol). The mixture was stirred for 18 h at 95° C., before being cooled down, filtered over silica, and washed with 1,4-dioxane. The filtrate was evaporated, and purification by FC (SiO₂; PE/EtOAc) gave the title compound (1.1 g, mmol, 42.28% yield) as a light yellow oil. MS (ESI): m/z=312.1 [M-tBu+H]⁺

### Example B.254

### 2-[4-(azetidin-3-yl)phenyl]benzamide; 4-methylbenzenesulfonic Acid

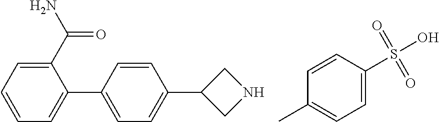

A solution of tert-butyl 3-[4-(2-carbamoylphenyl)phenyl]azetidine-1-carboxylate (350.0 mg, 0.990 mmol) and p-toluenesulfonic acid monohydrate (377.82 mg, 1.99 mmol) in EtOAc (30 mL) was stirred at 80° C. for 3 h, before being evaporated. Purification by RP-HPLC gave the title compound (166.7 mg, 38.75% yield) as a yellow viscous oil. MS (ESI): m/z=253.2 [M+H]⁺

### Step a): Tert-Butyl 3-(4-bromophenyl)azetidine-1-carboxylate

To a mixture of 4-bromophenylboronic acid (CAS RN: 5467-74-3; 200.04 g, 996.08 mmol) and tert-butyl 3-iodoazetidine-1-carboxylate (CAS RN: 254454-54-1; 141.0 g. 498.04 mmol) in 2-propanol (500 mL) was added rac-(I R,2R)-2-aminocyclohexan-1-ol (3.44 g, 29.88 mmol), and nickel(II) iodide (9.34 g, 29.88 mmol). A mixture of sodium bis(trimethylsilyl)amide in THE (1 L, 1000 mmol) was added slowly to the reaction mixture, under N₂ and keeping the temperature below 30° C. After the resulting mixture was stirred at 25° C. for 30 min, the mixture was heated to 80° C. and stirred for 12 h. The reaction mixture was poured onto H₂O (3 L) and EtOAc (3 L), and the layers were separated. The aqueous layer was extracted twice with EtOAc (2×2 L). The combined organic layers were evaporated, and purified by FC (SiO₂; PE/EtOAc), to give the title compound (140 g, 448.43 mmol, 90.04% yield) as an off-white oil. MS (ESI): m/z=256.1 [M−tBu+H]⁺

### Step b): Tert-Butyl 3-[4-(2-methoxycarbonylphenyl)phenyl]azetidine-1-carboxylate

A solution of tert-butyl 3-(4-bromophenyl)azetidine-1-carboxylate (2.1 g, 6.73 mmol), 2-methoxycarbonylphenylboronic acid (1.69 g, 9.42 mmol) and cesium carbonate (3.29 g, 10.09 mmol) in 1,4-Dioxane (55 mL) and Water (3 mL) was sparged with Argon for 5 min, before being treated with 1,1′-bis(diphenylphosphino)ferrocene-palladium(II)dichloride dichloromethane complex (439.1 mg, 0.540 mmol). The mixture was stirred for 18 h at 95° C., before being cooled down, filtered over silica, and washed with 1,4-dioxane. The filtrate was evaporated, and purification by FC (SiO₂; PE/EtOAc) gave the title compound (1.1 g, mmol, 42.28% yield) as a light yellow oil. MS (ESI): m/z=312.1 [M-tBu+H]⁺

### Step c): 2-[4-(1-tert-butoxycarbonyl azetidin-3 yl)phenyl]benzoic Acid

To a solution of tert-butyl 3-[4-(2-methoxycarbonylphenyl)phenyl]azetidine-1-carboxylate (600.0 mg, 1.63 mmol) in 4:4:1 THF/MeOH/water (22.5 mL) was added lithium hydroxide monohydrate (342.59 mg, 8.16 mmol). The mixture was stirred for 18 h at 25° C., before being evaporated and acidified to pH 4 with a saturated solution of citric acid. The resulting suspension was stirred for 30 min and filtered. The cake was washed with water and dried, to give the title compound (350 mg, 57.62% yield) as a white solid. MS (ESI): m/z=352.2 [M−H]⁻

### Step d): Tert-Butyl 3-[4-(2-carbamoylphenyl)phenyl]azetidine-1-carboxylate

A solution of 2-[4-(1-tert-butoxycarbonylazetidin-3-yl)phenyl]benzoic acid (400.0 mg, 1.13 mmol) in THE (10 mL) was treated with CDI (162.15 mg, 1.47 mmol), at 23° C. The mixture was stirred for 3 h at this temperature, before being treated with ammonium hydroxide (3 mL, 29% aqueous solution). The mixture was stirred for 18 h at this temperature, before being evaporated. The residue was partitioned between EtOAc (50 mL) and water (20 mL). The organic layer was dried over Na₂SO₄, filtered, and evaporated, to give the title compound (350 mg, 83.36% yield) as a white waxy solid. MS (ESI): m/z=350.8 [M−H]⁻

### Example B.255

### N-[[3-(azetidin-3-yl)-1-bicyclo[1.1.1]pentanyl]methyl]-1-(trifluoromethyl)cyclopropanamine; 4-methylbenzenesulfonic Acid

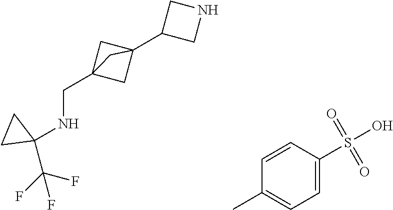 [SMILES: FC(F)(F)C1(NCC23CC(C4CNC4)(C2)C3)CC1 ; Cc1ccc(S(=O)(=O)O)cc1]

A solution of tert-butyl 3-[3-[[[1-(trifluoromethyl)cyclopropyl]amino]methyl]-1-bicyclo[1.1.1]pentanyl]azetidine-1-carboxylate (380.0 mg, 1.05 mmol) in EtOAc (25 mL) was treated with p-toluenesulfonic acid monohydrate (441.23 mg, 2.32 mmol), at 23° C. The mixture was stirred for 16 h at 50° C., before being cooled down to 0° C. for 1 h. The resulting precipitate was collected by filtration and washed twice with MTBE (2*100 mL), to give the title compound (607.1 mg, 90.46% yield) as a light yellow solid. MS (ESI): m/z=261.2 [M+H]⁺

### Step a): Tert-Butyl 3-[3-(hydroxymethyl)-1-bicyclo[1.1.1]pentanyl]azetidine-1-carboxylate

A solution of 3-(1-tert-butoxycarbonylazetidin-3-yl)bicyclo[1.1.1]pentane-1-carboxylic acid (CAS RN: 2227205-20-9; 6.0 g, 22.45 mmol) in THE (70 mL) was treated with borane-methyl sulfide complex (4262.87 mg, 56.11 mmol), at 0° C. The mixture was stirred at reflux for 6 h, before being cooled down to 0° C., treated dropwise with MeOH (5 mL), and evaporated. The residue was diluted with brine, and extracted with EtOAc (3×). The organic layers were combined, dried over Na₂SO₄, filtered and evaporated, to give the title compound (5.4 g, 90.22% yield) as a white solid. MS (ESI): m/z=198.0 [M-Boc+H]⁺

### Step b): Tert-Butyl 3-(3 formyl-1-bicyclo[1.1.1]pentanyl)azetidine-1-carbonylate

To a solution of tert-butyl 3-[3-(hydroxymethyl)-1-bicyclo[1.1.1]pentanyl]azetidine-1-carboxylate (1.2 g, 4.74 mmol) in DCM (50 mL) was added DMP (2.4 g, 5.68 mmol), at 0° C. The mixture was allowed to warm up to 23° C. and was stirred for 3 h at this temperature. The mixture was treated with saturated aqueous sodium sulfite (40 mL) and extracted with DCM (2×20 mL), washed with saturated aqueous NaHCO₃ and brine (2×20 mL), dried over Na₂SO₄, filtered and evaporated, to give the title compound (1.1 g, 64.68% yield) as a colorless viscous oil, which was directly used in the next step.

### Step c): Tert-Butyl 3-[3-[[[1-(trifluoromethyl)cyclopropyl]amino]methyl]-1-bicyclo[1.1.1]pentanyl]azetidine-1-carboxylate

A solution of tert-butyl 3-(3-formyl-1-bicyclo[1.1.1]pentanyl)azetidine-1-carboxylate (1.1 g, 3.06 mmol) and 1-(trifluoromethyl)cyclopropanamine hydrochloride (544.46 mg, 3.37 mmol) in 1,2-dichloroethane (50 mL) was stirred stirred for 2 h at 23° C., before being treated with sodium triacetoxyborohydride (1.9 g, 9.19 mmol). The mixture was stirred at this temperature for another 18 h, before being treated with saturated aqueous NaHCO₃. The mixture was extracted with DCM. The combined organic layers were dried over Na₂SO₄, filtered, and evaporated, to give the title compound (380 mg, 34.41% yield) as a clear oil. MS (ESI): m/z=361.2 [M+H]⁺

### Example B.256

### 3-(azetidin-3-yl)-N-[[11-(trifluoromethyl)cyclopropyl]methyl]bicyclo[1.1.1]pentan-1-amine; 4-methylbenzenesulfonic Acid

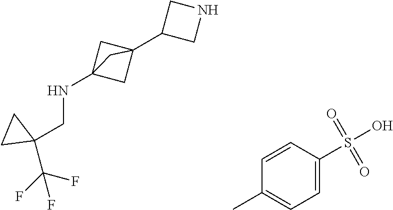 [SMILES: FC(F)(F)C1(CNC23CC(C4CNC4)(C2)C3)CC1 ; Cc1ccc(S(=O)(=O)O)cc1]

A solution of tert-butyl 3-[3-[[1-(trifluoromethyl)cyclopropyl]methylamino]-1-bicyclo[1.1.1]pentanyl]azetidine-1-carboxylate (800.0 mg, 2.22 mmol) in EtOAc (50 mL) was treated with p-toluenesulfonic acid monohydrate (928.91 mg, 4.88 mmol), at 23° C. The mixture was stirred for 16 h at this temperature, before being cooled down to 0° C. for 1 h. The precipitate was filtered, washed with MTBE, and dried, to give the title compound (1191 mg, 84.26% yield) as a white solid. MS (ESI): m/z=261.2 [M+H]⁺

### Step a): Tert-Butyl 3-[3-(benzyloxycarbonylamino)-1-bicyclo[1.1.1]pentanyl]azetidine-1-carboxylate

A solution of 3-(1-tert-butoxycarbonylazetidin-3-yl)bicyclo[1.1.1]pentane-1-carboxylic acid (CAS RN: 2227205-20-9; 2.0 g, 7.48 mmol) and benzyl alcohol (1.6 g, 14.96 mmol) in toluene (50 mL) was treated with TEA (3.13 mL, 22.45 mmol), at 23° C. The mixture was stirred for 5 min at this temperature, and diphenylphosphonic azide (1.69 mL, 7.86 mmol) was added. The mixture was stirred for another 15 min at 23° C., and for 16 h at 100° C. The mixture was cooled down, poured into ice-cold water (50 mL), and extracted with MTBE. The organic layer was washed with water and brine, dried over Na₂SO₄, filtered, and evaporated. Purification by FC (SiO₂; hexane/MTBE) gave the title compound (2.35 g, 80.11% yield) as a colorless viscous oil. MS (ESI): m/z=273.0 [M-Boc+H]⁺

### Step b): Tert-Butyl 3-(3-amino-1-bicyclo[1.1.1]pentanyl)azetidine-1-carboxylate

To a solution of tert-butyl 3-[3-(benzyloxycarbonylamino)-1-bicyclo[1.1.1]pentanyl]azetidine-1-carboxylate (2200.0 mg, 5.91 mmol) in MeOH (50 mL) was added palladium (10% on C) (0.21 mL, 0.210 mmol). The reaction mixture was stirred for 24 h at 23° C. under hydrogen atmosphere, before being put back under Ar and filtered. The filtrate was evaporated, and the residue was triturated with MTBE (100 mL). The precipitate was collected by filtration, to give the title compound (600 mg, 40.49% yield) as a white solid. MS (ESI): m/z=239.2 [M+H]

### Step c): Tert-Butyl 3-[3-[[1-(trifluoromethyl)cyclopropyl]methylamino]-1-bicyclo[1.1.1]pentanyl]azetidine-1-carboxylate

A solution of tert-butyl 3-(3-amino-1-bicyclo[1.1.1]pentanyl)azetidine-1-carboxylate (1.0 g, 4.2 mmol) and 1-(trifluoromethyl)cyclopropanecarbaldehyde (521.47 mg, 3.78 mmol) in 1,2-dichloroethane (50 mL) was stirred for 2 h at 23° C., before being treated with sodium triacetoxyborohydride (2.6 g, 12.59 mmol). The mixture was stirred for another 18 h at this temperature, before being treated with saturated aqueous NaHCO₃. The mixture was extracted with DCM (2×20 mL), and the combined organic layers were dried over Na₂SO₄, filtered, and evaporated. Purification by FC (SiO₂; hexane/MTBE) gave the title compound (800 mg, 50.26% yield) as a white solid. MS (ESI): m/z=361.2 [M+H]⁺

### Example B.257

### 2-[3-(azetidin-3-yl)-1-bicyclo[1.1.1]pentanyl]-5-(2,2-dimethylpropyl)-1,3,4-oxadiazole; 2,2,2-trifluoroacetic Acid

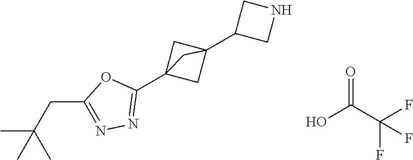 [SMILES: CC(C)(C)Cc1nnc(C23CC(C4CNC4)(C2)C3)o1 ; O=C(O)C(F)(F)F]

To a solution of 3-[3-(5-neopentyl-1,3,4-oxadiazol-2-yl)-1-bicyclo[1.1.1]pentanyl]azetidine-1-carboxylic acid tert-butyl ester (260 mg, 0.683 mmol) in DCM (3 mL) was added TFA (526.42 μL, 6.83 mmol), at 23° C. The mixture was stirred for 18 h at this temperature, before being evaporated, to give the title compound (425 mg, quant.). MS (ESI): m/z=262.2 [M+H]⁺

### Step a): 3-[3-[(3,3-dimethylbutanoylamino)carbamoyl]-1-bicyclo[1.1.1]pentanyl]azetidine-1-carboxylic Acid Tert-Butyl Ester

A solution of 3-(1-tert-butoxycarbonylazetidin-3-yl)bicyclo[1.1.1]pentane-1-carboxylic acid (CAS RN: 2227205-20-9; 250 mg, 0.935 mmol) in DCM (5 mL) was treated with CDI (159.23 mg, 0.982 mmol), at 0° C. The mixture was stirred for 15 min at this temperature, and for another 45 min at 23° C., before being treated with 3,3-dimethylbutyrohydrazide (133.93 mg, 1.03 mmol). The mixture was stirred for 18 h at 23° C., before being diluted with DCM. The organic layer was washed with 1 M Na₂CO₃ aqueous solution, dried over Na₂SO₄, filtered, and evaporated, to give the crude title compound (375 mg, quant.). MS (ESI): m/z=324.2 [M-tBu+H]⁺

### Step b): 3-[3-(5-neopentyl-1,3,4-oxadiazol-2-yl)-1-bicyclo[1.1.1]pentanyl]azetidine-1-carbolic Acid Tert-Butyl Ester

To a solution of 3-[3-[(3,3-dimethylbutanoylamino)carbamoyl]-1-bicyclo[1.1.1]pentanyl]azetidine-1-carboxylic acid tert-butyl ester (375 mg, 0.929 mmol) in THE (8 mL) under Ar was added Burgess reagent (553.38 mg, 2.32 mmol), at 23° C. The mixture was heated to 70° C. and stirred for 18 h at this temperature, before being partitioned between EtOAc and a 1 M Na₂CO₃ aqueous solution. The organic phase was collected and the aqueous phase was back-extracted with ethyl acetate. The combined organic phases were dried over Na₂SO₄, filtered, and evaporated. Purification by FC (SiO₂; heptane/EtOAc) gave the title compound (264 mg, 75% yield). MS (ESI): m/z=362.3 [M+H]⁺

In analogy to Example B.257, the following building blocks were generated using the relevant commercial building blocks and 3-(1-tert-butoxycarbonylazetidin-3-yl)bicyclo[1.1.1]pentane-1-carboxylic acid (CAS RN: 1211526-53-2) or 3-(1-tert-butoxycarbonyl-4-piperidyl)bicyclo[1.1.1]pentane-1-carboxylic acid (CAS RN: 2020070-00-0) in Step a).

| | | | | MS, |
|---|---|---|---|---|
| | | | Building | ESI: |
| Ex. | Structure | Systematic Name | Blocks | m/z |
| M.2 | 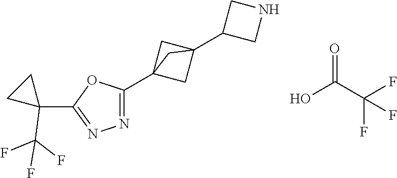 [SMILES: FC(F)(F)C1(c2nnc(C34CC(C5CNC5)(C3)C4)o2)CC1 ; O=C(O)C(F)(F)F] | 2-[3-(azetidin-3-y1)-1- bicyclo[1.1.1]pentanyl 1-5-[1- (trifluoromethyl)cyclo propyl]-1,3,4- oxadiazole;2,2,2- trifluoroacetic acid | 1-(trifluoro- methyl) cyclopropane- 1-carbo- hydrazide (CAS RN: 1016557-86-0) | 300.2 [M + H]⁺ |
| M.18 | 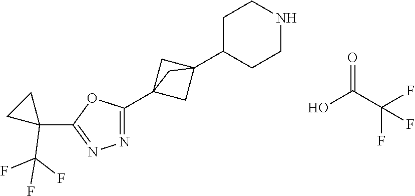 [SMILES: FC(F)(F)C1(c2nnc(C34CC(C5CCNCC5)(C3)C4)o2)CC1 ; O=C(O)C(F)(F)F] | 2-[3-(4-piperidyl)-1- bicyclo[1.1.1]pentanyl 1-5-[1- (trifluoromethyl) cyclopropyl]-1,3,4- oxadiazole;2,2,2- trifluoroacetic acid | 1-(trifluoro- methyl) cyclopropane- 1-carbo- hydrazide (CAS RN: 1016557-86-0) | 328.2 [M + H]⁺ |
| M.24 | 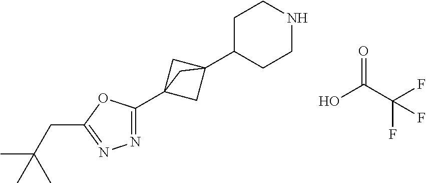 [SMILES: CC(C)(C)Cc1nnc(C23CC(C4CCNCC4)(C2)C3)o1 ; O=C(O)C(F)(F)F] | 2-(2,2- dimethylpropyl)-5-[3- (4-piperidy1)-1- bicyclo[1.1.1]pentanyl. 1-1,3,4- oxadiazole;2,2,2- trifluoroacetic acid | 3,3- dimethylbutanohydrazide (CAS RN: 712303-26-9) | 290 [M + H]⁺ |

### Example B.258

### 2-[3-(azetidin-3-yl)-1-bicyclo[1.1.1]pentanyl]-5-(2,2,2-trifluoroethyl)-1,3,4-oxadiazole; 2,2,2-trifluoroacetic Acid

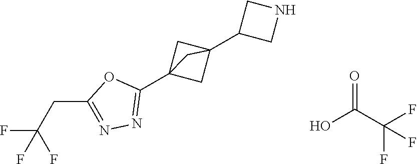 [SMILES: FC(F)(F)Cc1nnc(C23CC(C4CNC4)(C2)C3)o1 ; O=C(O)C(F)(F)F]

To a solution of tert-butyl 3-[3-[5-(2,2,2-trifluoroethyl)-1,3,4-oxadiazol-2-yl]-1-bicyclo[1.1.1]pentanyl]azetidine-1-carboxylate (2.0 g, 5.36 mmol) in DCM (200 mL) was added TFA (2.06 mL, 26.78 mmol), at 25° C. The mixture was stirred for 18 h at this temperature, before being evaporated. The residue was treated with TBME (200 mL), and the resulting precipitate was filtered, washed with TBME (2×50 mL), and dried, to give the title compound (1.8 g, 81.3% yield) as a light yellow solid. MS (ESI): m/z=274.0 [M+H]⁺

### Step a): Tert-Butyl 3-[3-[(3,3,3-trifluoropropanoylamino)carbamoyl]-1-bicyclo[1.1.1]pentanyl]azetidine-1-carboxylate

To a stirred solution of 3-(1-tert-butoxycarbonylazetidin-3-yl)bicyclo[1.1.1]pentane-1-carboxylic; acid (CAS RN: 1211526-53-2; 4.0 g, 14.96 mmol) in DCM (120 mL) was added CDI (2.79 g, 17.21 mmol), at 25° C. The mixture was stirred for 30 min at this temperature, before being treated with 3,3,3-trifluoropropanehydrazide (CAS RN: 934171-99-0; 2.23 g, 15.71 mmol). The mixture was stirred for another 18 h, before being diluted with DCM (150 mL). The organic layer was washed with water, dried over Na₂SO₄, filtered and evaporated, to give the title compound (5.8 g, 97.05% yield) as a white solid. MS (ESI): m/z=390.2 [M−H]⁻

### Step b): Tert-Butyl 3-[3-[5-(2,2,2-trifluoroethyl)-1,3,4-oxadiazol-2-yl]-1-bicyclo[1.1.1]pentanyl]azetidine-1-carboxylate

To a stirred solution of tert-butyl 3-[3-[(3,3,3-trifluoropropanoylamino)carbamoyl]-1-bicyclo[1.1.1]pentanyl]azetidine-1-carboxylate (5.8 g, 14.82 mmol) and DIPEA (7.74 mL, 44.46 mmol) in ACN (200 mL) was added p-toluenesulfonyl chloride (3.67 g, 19.26 mmol). The mixture was stirred for 24 h at 50° C., before being evaporated. Purification by FC (SiO₂; PE/TBME) gave the title compound (4.2 g, 72.11% yield) as a light brown solid. MS (ESI): m/z=318.0 [M−tBu+H]⁺

In analogy to Example B.258, the following building block was generated using the relevant commercial building blocks and 3-(1-tert-butoxycarbonylazetidin-3-yl)bicyclo[1.1.1]pentane-1-carboxylic acid (CAS RN: 1211526-53-2) in Step a).

| | | | Building | MS, ESI: |
|---|---|---|---|---|
| Ex. | Structure | Systematic Name | Blocks | m/z |
| B.259 | 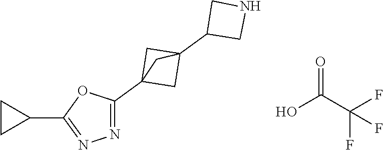 [SMILES: C1CC1c1nnc(C23CC(C4CNC4)(C2)C3)o1 ; O=C(O)C(F)(F)F] | 2-[3-(azetidin-3-yl)-1- bicyclo[1.1.1]pentanyl 1-5-cyclopropyl-1,3,4- oxadiazole;2,2,2- trifluoroacetic acid | cyclopropane- carbohydrazide (CAS RN: 6952-93-8) | 232.2 [M + H]⁺ |

### Example B.261

### 3-[[2-fluoro-4-(trifluoromethyl)phenoxy]methyl]azetidine; 2,2,2-trifluoroacetic Acid

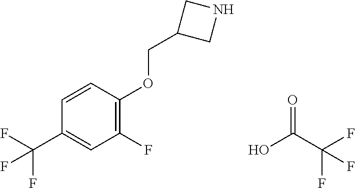 [SMILES: Fc1cc(C(F)(F)F)ccc1OCC1CNC1 ; O=C(O)C(F)(F)F]

A solution of tert-butyl 3-((2-fluoro-4-(trifluoromethyl)phenoxy)methyl)azetidine-1-carboxylate (35 mg, 100 μmol) in DCM (501 μL) was treated with TFA (154 μL, 2 mmol), at 23° C. The mixture was stirred for 4 h at this temperature before being evaporated, to give the title compound (40.7 mg, 101% yield) as a white solid. MS (ESI): m/z=250.1 [M+H]⁺

### Step a): Tert-Butyl 3-[[2 ffluoro-4-(trifluoromethyl)phenoxy]methyl]azetidine-1-carboxylate

A solution of 2-fluoro-4-(trifluoromethyl)phenol (CAS RN: 77227-78-2; 481 mg, 2.67 mmol) in DCM (13.4 mL) was treated with tert-butyl 3-(hydroxymethyl)azetidine-1-carboxylate (CAS RN: 142253-56-3; 500 mg, 2.67 mmol), triphenylphosphine (770 mg, 2.94 mmol), and DIAD (594 mg, 571 μl, 2.94 mmol), at 23° C. The mixture was stirred for 16 h at this temperature, before being treated with a saturated aqueous solution of NaHCO₃ (20 mL). The phases were separated, and the aqueous layer was extracted with DCM (2×). The combined organic layers were dried over Na₂SO₄, filtered, and evaporated. The crude residue was dissolved in EtOH (6 mL) and treated with a homogeneous solution of zinc chloride (218 mg, 1.6 mmol) in EtOH (2 mL. 0.5 M). The mixture was stirred for 30 min, during which a white solid precipitated. The precipitate was filtered off, and the filtrate evaporated. The filtrate was purified by FC (SiO₂; heptane/EtOAc), to give the title compound (592 mg, 60.3% yield) as a white solid. MS (ESI): m/z=294.0 [M-tBu+H]⁺

In analogy to Example B.261, the following building block was generated using the relevant commercial building block and tert-butyl 3-(hydroxymethyl)azetidine-1-carboxylate (CAS RN: 142253-56-3) in Step a). Deprotection could also be performed with 4M HCl in 1,4-dioxane.

| | | | Building | MS, ESI: |
|---|---|---|---|---|
| Ex. | Structure | Systematic Name | Blocks | m/z |
| B.267 | 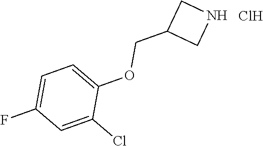 [SMILES: Fc1ccc(OCC2CNC2)c(Cl)c1 ; Cl] | 3-[(2-chloro-4-fluoro- phenoxy)methyl] azetidine; hydrochloride | 2-Chloro-4- fluorophenol (CAS RN: 1996-41-4) | 216.1 [M + H]⁺ |

### Example B.263

### 3-[2-[2-fluoro-4-(trifluoromethyl)phenyl]ethyl]azetidine; 2,2,2-trifluoroacetic Acid

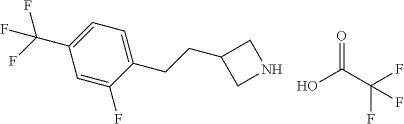 [SMILES: Fc1cc(C(F)(F)F)ccc1CCC1CNC1 ; O=C(O)C(F)(F)F]

A solution of tert-butyl 3-(2-fluoro-4-(trifluoromethyl)phenethyl)azetidine-1-carboxylate (600 mg, 1.73 mmol) in DCM (5 mL) was treated with TFA (1.33 mL, 17.3 mmol), at 23° C. The mixture was stirred for 3 h at this temperature, before being evaporated, to give the title compound (732 mg, quant.) as a crude colorless liquid. MS (ESI): m/z=248.2 [M+H]⁺

### Step a): Diethyl (2-fluoro-4-(trifluoromethyl)benzyl)phosphonate

A solution of 1-(bromomethyl)-2-fluoro-4-(trifluoromethyl)benzene (CAS RN: 239087-07-1; 1.1 g, 4.28 mmol) in triethyl phosphite (1.83 mL, 10.7 mmol) was stirred at reflux for 3 h. The clear and colorless mixture was directly purified by FC (SiO₂; heptane/EtOAc), to give the title compound (0.833 g; 62%) as a colorless oil. MS (ESI): m/z=315.2 [M+H]⁺

### Step b): Tert-Butyl (E)-3-(2-fluoro-4-(trifluoromethyl)styryl)azetidine-1-carboxylate

A suspension of NaH 55% in mineral oil (122 mg, 2.8 mmol) in THF (5 mL) was treated dropwise with a solution of diethyl (2-fluoro-4-(trifluoromethyl)benzyl)phosphonate (800 mg, 2.55 mmol) in THF (5 mL), at 0° C. The mixture was stirred for 30 min at this temperature, before being added dropwise to a solution of tert-butyl 3-formylazetidine-1-carboxylate (CAS RN: 177947-96-5; 472 mg, 2.55 mmol) in THE (2.5 mL), at 0° C. The mixture was stirred for another 3 h at 0° C., before being poured into water and EtOAc. The layers were separated, and the organic layer was washed with brine, dried over MgSO₄, filtered, and evaporated. Purification by FC (SiO₂; heptane/EtOAc) gave the title compound (607 mg, 69% yield) as a colorless oil. MS (ESI): m/z=290.1 [M-tBu+H]⁺

### Step c): Tert-Butyl 3-(2-fluoro-4-(trifluoromethyl)phenethyl)azetidine-1-carbonylate

To a solution of tert-butyl (E)-3-(2-fluoro-4-(trifluoromethyl)styryl)azetidine-1-carboxylate (607 mg, 1.76 mmol) in 1:1 MeOH/EtOAc (14 mL) was added Pd/C 10% (60 mg, 1.76 mmol). The mixture was stirred for 4 h at 23° C., under H₂ atmosphere, before being filtered. The filtrate was evaporated, to give the title compound (0.61 g; 98.0%) as a colorless oil. MS (ESI): m/z=292.2 [M-tBu+H]⁺

### Example B.265

### N-[5-(trifluoromethyl)pyrazin-2-yl]-2-azaspiro[3.5]nonan-7-amine; 4-methylbenzenesulfonic Acid

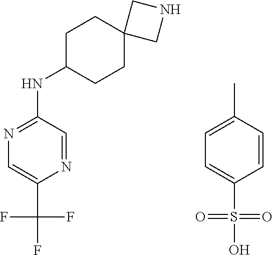 [SMILES: FC(F)(F)c1cnc(NC2CCC3(CC2)CNC3)cn1 ; Cc1ccc(S(=O)(=O)O)cc1]

To a solution of 7-[[5-(trifluoromethyl)pyrazin-2-yl]amino]-2-azaspiro[3.5]nonane-2-carboxylic acid tert-butyl ester (525 mg, 1.36 mmol) in isopropyl acetate (12 mL) was added p-toluenesulfonic acid monohydrate (568.57 mg, 2.99 mmol). The mixture was stirred at 80° C. for 5 h, before being evaporated. Et₂O was added, and the resulting precipitate was filtered off, washed with Et₂O (2×), and dried, to give the title compound (755 mg, 83.7%) as a white solid MS (ESI) m/z=287.1 [M+H]⁺

### Step a): Tert-Butyl 7-[[5-(trifluoromethyl)pyrazin-2-yl]amino]-2-azaspiro[3.5]nonane-2-carboxylate

To a solution of 7-amino-2-azaspiro[3.5]nonane-2-carboxylic acid tert-butyl ester (CAS RN: 1408075-19-3; 1.33 g, 5.53 mmol) and DIPEA (1.45 mL, 8.3 mmol) in DMSO (10 mL) was added 2-fluoro-5-(trifluoromethyl)pyrazine (CAS RN: 1220799-65-4; 919.06 mg, 5.53 mmol). The mixture was stirred at 60° C. for 5 h, before being poured into EtOAc. The mixture was washed with water and brine. The organic layer was dried over Na₂SO₄, filtered, and evaporated. Purification by FC (SiO₂; heptane/EtOAc) gave the title compound (1.66 g, 73.75% yield) as a white solid. MS (ESI): m/z=331.2 [M+H-Buten]⁺

### Example B.266

### 1-(2-azaspiro[3.3]heptan-6-yl)-N-[[1-(trifluoromethyl)cyclopropyl]methyl]methanamine; 4-methylbenzenesulfonic Acid

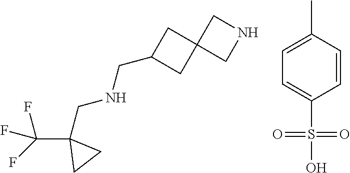

A solution of tert-butyl 6-[[[1-(trifluoromethyl)cyclopropyl]methylamino]methyl]-2-azaspiro[3.3]heptane-2-carboxylate; 4-methylbenzenesulfonic acid (450.0 mg, 0.860 mmol) in ACN (15 mL) was treated with PTSA (328.84 mg, 1.73 mmol), at 23° C. The reaction mixture was stirred for 24 h at 70° C., before being evaporated. Trituration with i-PrOH gave the title compound (190 mg, 37.09% yield) as a white solid. MS (ESI): m/z=249.2 [M+H]⁺

### Step a): Tert-Butyl 6-[[1-(trifluoromethyl)cyclopropyl]methylcarbamoyl]-2-azaspiro[3.3]heptane-2-carboxylate

To a stirred solution of 2-tert-butoxycarbonyl-2-azaspiro[3.3]heptane-6-carboxylic acid (CAS RN: 1211526-53-2; 1.0 g, 4.14 mmol) and [1-(trifluoromethyl)cyclopropyl]methanamine; hydrochloride (CAS RN: 1783418-59-6; 727.7 mg, 4.14 mmol) in DMF (5 mL) were added HATU (1.89 g, 4.97 mmol) and DIPEA (2.53 mL, 14.51 mmol). The mixture was stirred for 18 h at 23° C., before being poured into water and extracted with EtOAc (2×50 mL). The combined organic layers were washed with water, dried over Na₂SO₄, filtered, and evaporated. Trituration with MTBE gave the title compound (1 g, 2.76 mmol, 63.25% yield) as a white solid. MS (ESI): m/z=361.0 [M−H]⁻

### Step b): Tert-Butyl 6-[[[1-(trifluoromethyl)cyclopropyl]methylamino]methyl]-2-azaspiro[3.3]heptane-2-carboxylate

A solution of tert-butyl 6-[[1-(trifluoromethyl)cyclopropyl]methylcarbamoyl]-2-azaspiro[3.3]heptane-2-carboxylate (1.0 g, 2.76 mmol) in THE (30 mL) was treated with borane-methyl sulfide complex (628.9 mg, 8.28 mmol), at 0° C. The mixture was stirred at reflux for 6 h, before being cooled down to 0° C., treated dropwise with MeOH (5 mL), and evaporated. The residue was diluted with brine and extracted with EtOAc (3×). The combined organic layers were dried over Na₂SO₄, filtered, and evaporated. The residue was dissolved in MTBE (30 mL), and PTSA was added to adjust the pH to 4. The resulting precipitate was collected by filtration, to give the title compound (450 mg, 29.76°% yield) as a white solid. MS (ESI): m/z=349 [M+H]⁺

In analogy to Example B.266, the following building block was generated using the relevant commercial building block and of 2-tert-butoxycarbonyl-2-azaspiro[3.3]heptane-6-carboxylic acid (CAS RN: 1211526-53-2) in Step a).

| | | | Building | MS, ESI: |
|---|---|---|---|---|
| Ex. | Structure | Systematic Name | Blocks | m/z |
| B.271 | 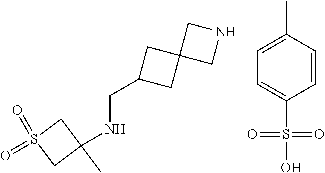 | 3 N-(2- azaspiro[3.3]heptan-6- ylmethyl)-3-methyl- 1,1-dioxo-thietan-3- amine;4- methylbenzenesulfonic acid | 3-methyl-1,1- dioxo-thietan- 3- amine; hydrochloride (CAS RN: 2225141- 46-6) | 245.0 [M + H]⁺ |
| B.276 | 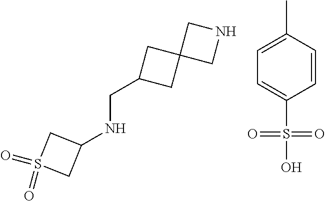 | N-(2- azaspiro[3.3]heptan-6- ylmethyl)-1,1-dioxo- thietan-3-amine;4- methylbenzenesulfonic acid | 3- Aminothietane 1,1-Dioxide hydrochloride (CAS RN: 1422344-24-8) | 231.4 [M + H]⁺ |

### Example B.268

### N-(2-azaspiro[3.3]heptan-6-ylmethyl)-3-(trifluoromethyl)oxetan-3-amine; 4-methylbenzenesulfonic Acid

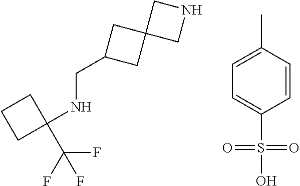 [SMILES: FC(F)(F)C1(NCC2CC3(CNC3)C2)CCC1 ; Cc1ccc(S(=O)(=O)O)cc1]

A solution of tert-butyl 6-[[[3-(trifluoromethyl)oxetan-3-yl]amino]methyl]-2-azaspiro[3.3]heptane-2-carboxylate (270.0 mg, 0.770 mmol) in 4:1 MTBE/ACN (25 mL) was treated with PTSA (366.45 mg, 1.93 mmol), at 23° C. The mixture was stirred for 18 h at 40° C., and the resulting precipitate was filtered off, washed with Et₂O and dried, to give the title compound (187.7 mg, 38.9% yield) as a white solid. MS (ESI): m/z=251.0 [M+H]⁺

### Step a): Tert-Butyl 6-[[[3-(trifluoromethyl)oxetan-3-yl]amino]methyl]-2-azaspiro[3.3]heptane-2-carboxylate

A solution of 3-(trifluoromethyl)oxetan-3-amine; hydrochloride (394.07 mg, 2 mmol) in DCE (15 mL) was treated with tert-butyl 6-formyl-2-azaspiro[3.3]heptane-2-carboxylate (CAS RN: 1440960-67-7; 300.0 mg, 1.3 mmol) and TEA (0.24 mL, 1.73 mmol), at 23° C. The mixture was stirred for 10 min at this temperature, before being treated with acetic acid (159.94 mg, 2.66 mmol). The mixture was stirred for another 60 min, and sodium triacetoxyborohydride (705.59 mg, 3.33 mmol) was added. The mixture was stirred for another 18 h, before being treated with saturated aqueous NaHCO₃. The mixture was extracted with DCM (2×50 mL). The combined organic layers were washed with brine, dried over Na₂SO₄, filtered, and evaporated, to give the title compound (390 mg, 71.05% yield) as a light yellow solid. MS (ESI): m/z=351.2 [M+H]⁺

### Example B.270

### 6-(2-chloro-4-fluoro-phenyl)sulfonyl-2-azaspiro[3.3]heptane; 2,2,2-trifluoroacetic Acid

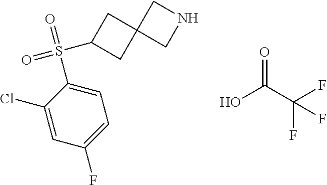 [SMILES: O=S(=O)(c1ccc(F)cc1Cl)C1CC2(CNC2)C1 ; O=C(O)C(F)(F)F]

A solution of tert-butyl 6-((2-chloro-4-fluorophenyl)sulfonyl)-2-azaspiro[3.3]heptane-2-carboxylate (209 mg, 536 μmol) in DCM (2 mL) was treated with TFA (207 μL, 2.68 mmol), at 23° C. The mixture was stirred for 16 h at this temperature, before being evaporated, to give the title compound (195 mg, 90.1% yield) as a white solid. MS (ESI): m/z=290.1 [M+H]⁺

### Step a): Tert-Butyl 6-((methylsulfonyl)oxy)-2-azaspiro[3.3]heptane-2-carboxylate

To a solution of tert-butyl 6-hydroxy-2-azaspiro[3.3]heptane-2-carboxylate (CAS RN: 1147557-97-8; 1 g, 4.69 mmol) in dry DCM (11.8 mL) was added TEA (1.31 mL, 9.38 mmol). The mixture was cooled to 0° C. and methanesulfonyl chloride (401 μL, 5.16 mmol) was added. The mixture was stirred for 4 h at 23° C., before being washed with water and brine. The organic layer was dried over Na₂SO₄, filtered, and evaporated, to give the title compound (1.355 g, 97.2% yield) as an orange solid. MS (ESI): m/z=236.2 [M-tBu+H]⁺

### Step b): Tert-Butyl 6-((2-chloro-4-fluorophenyl)thio)-2-azaspiro[3.3]heptane-2-carboxylate

A solution of tert-butyl 6-((methylsulfonyl)oxy)-2-azaspiro[3.3]heptane-2-carboxylate (537.5 mg, 1.84 mmol) in DMF (7 mL) was treated with 2-chloro-4-fluorobenzenethiol (250 mg, 1.54 mmol) and K₂CO₃ (425 mg, 3.07 mmol), at 23° C. under Ar. The mixture was heated to 80° C. and stirred for 4 h at this temperature, before being cooled down, diluted with EtOAc (20 mL), and washed with water (2×40 mL) and brine (1×40 mL). The organic layer was dried over MgSO₄, filtered, and evaporated. Purification by FC (SiO₂; heptane/EtOAc) gave the title compound (218 mg, 37.6% yield) as a colorless oil. MS (ESI): m/z=302.1 [M-tBu+H]⁺

### Step c): Tert-Butyl 6-((2-chloro-4-fluorophenyl)sulfonyl)-2-azaspiro[3.3]heptane-2-carboxylate

A solution of tert-butyl 6-((2-chloro-4-fluorophenyl)thio)-2-azaspiro[3.3]heptane-2-carboxylate (218 mg, 609 μmol) in DCM (8 mL) was treated portionwise with mCPBA (315 mg, 1.28 mmol) at 10-12° C. The cooling bath was removed, and the moisture was stirred at 20° C. for 3 h, before being diluted with DCM. The organic layer was washed with 5% NaHCO₃, water and brine, dried over MgSO₄, filtered, and evaporated. Purification by FC (SiO₂; heptane/EtOAc) gave the title compound (209.0 mg, 88% yield) as a white solid. MS (ESI): m/z=334.0 [M-tBu+H]⁺

### Example B.272

### N-(2-azaspiro[3.5]nonan-7-yl)-4,4-difluoro-cyclohezanesulfonamide; 4-methylbenzenesulfonic Acid

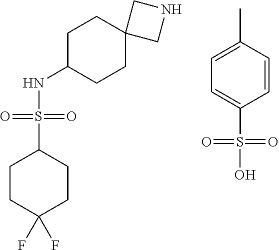 [SMILES: O=S(=O)(NC1CCC2(CC1)CNC2)C1CCC(F)(F)CC1 ; Cc1ccc(S(=O)(=O)O)cc1]

To a suspension of 7-[(4,4-difluorocyclohexyl)sulfonylamino]-2-azaspiro[3.5]nonane-2-carboxylic acid tert-butyl ester (310 mg, 0.734 mmol) in isopropyl acetate (2 mL) and tetrahydroftuan (8 mL) was added PTSA (209.34 mg, 1.1 mmol). The mixture was stirred at 100° C. for 6 h, before being cooled down and evaporated. The residue was treated with Et₂O, and the resulting precipitate was filtered off, washed with Et₂O, and dried, to give the title (335 mg, 87.7% yield) as a white solid. MS (ESI): m/z=323.1 [M+H]⁺

### Step a): Tert-Butyl 7-[(4,4-difluorocyclohexyl)sulfonylamino]-2-azaspiro[3.5]nonane-2-carboxylate

To a solution of 7-amino-2-azaspiro[3.5]nonane-2-carboxylic acid tert-butyl ester (CAS RN: 1408075-19-3; 0.250 g, 1.04 mmol) and DIPEA (272.5 μL, 1.56 mmol) in DCM (4.29 mL) was added 4,4-difluorocyclohexanesulfonyl chloride (CAS RN: 1196154-77-4; 227.44 mg, 1.04 mmol), at 0° C. The mixture was allowed to warm up to 23° C. and stirred for 15 h at this temperature, before being diluted with 1:1 DCM/Et₂O (10 mL). The resulting suspension was filtered off, washed with Et₂O (2×), and dried, to give the title compound (316 mg, 68.3% yield) as a white solid. MS (ESI): m/z=367.2 [M+H-Buten]⁺

### Example B.273

### N-[[1-(trifluoromethyl)cyclopropyl]methyl]-2-azaspiro[3.3]heptane-6-carboxamide; Hydrochloride

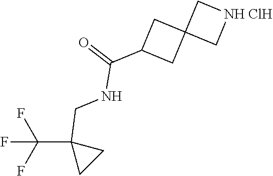

To a solution of benzyl 6-[[1-(trifluoromethyl)cyclopropyl]methylcarbamoyl]-2-azaspiro[3.3]heptane-2-carboxylate (500.0 mg, 1.26 mmol) in MeOH (10 mL) was added 10% Pd/C (0.04 mL, 0.380 mmol). The mixture was stirred for 48 h at 23° C. under hydrogen atmosphere. The solids were removed by filtration and the filtrate was evaporated. The residue was dissolved in THE (10 mL) and treated with 1,4-dioxane saturated with HCl, stirred for 10 min at 23° C. The precipitate was collected by filtration, to give the title compound (140.8 mg, 37.37% yield) as a white solid. MS (ESI): m/z=263.2 [M+H]⁺

### Step a): Benzyl 7-[(4,4-difluorocyclohexyl)sulfonylamino]-2-azaspiro[3.5]nonane-2-carboxylate

To a stirred solution of 2-benzyloxycarbonyl-2-azaspiro[3.3]heptane-6-carboxylic acid (CAS RN: 1291487-33-6; 550.0 mg, 2 mmol) and [1-(trifluoromethyl)cyclopropyl]methanamine; hydrochloride (CAS RN: 1783418-59-6; 350.78 mg, 2 mmol) in DMF (10 mL) were added HATU (987.52 mg, 2.6 mmol, 1.3 eq) and TEA (0.97 mL, 6.99 mmol). The mixture was stirred for 18 b at 23° C., before being poured onto water and extracted with EtOAc (2×50 mL). The combined organic layers were washed with water, dried over Na₂SO₄, and evaporated. Trituration with 50 mL of MTBE gave the title compound (600 mg, 75.76% yield) as a white solid. MS (ESI): m/z=397.2 [M+H]⁺

### Example B.274

### 3-(azetidin-3-yl)-1-[[4-(trifluoromethyl)phenyl]methyl]azetidine; 4-methylbenzenesulfonic Acid

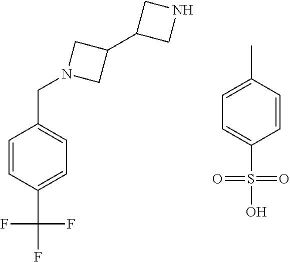 [SMILES: FC(F)(F)c1ccc(CN2CC(C3CNC3)C2)cc1 ; Cc1ccc(S(=O)(=O)O)cc1]

To a solution of 3-[l-[4-(trifluoromethyl)benzyl]azetidin-3-yl]azetidine-1-carboxylic acid tert-butyl ester (220 mg, 0.594 mmol) in isopropyl acetate (5 mL) was added PTSA (248.55 mg, 1.31 mmol). The mixture was stirred at 80° C. for 5 h, before being cooled down and filtered off. The white precipitate was washed with Et₂O (2×) and dried, to give the title compound (331 mg, 86.13%) as a white solid. MS (ESI): m/z=271.1 [M+H]⁺

### Step a): Tert-Butyl 3-[1-[[4-(trifluromethyl)phenyl]methyl]azetidin-3 yl]azetidine-1-carboxylate

To a solution of 4-(trifluoromethyl)benzaldehyde (CAS RN: 455-19-6; 246.06 mg, 1.41 mmol) and 3-(azetidin-3-yl)azetidine-1-carboxylic acid tert-butyl ester (CAS RN: 2007910-70-3; 0.300 g, 1.41 mmol) in 1,2-dichloroethane (2.5 mL) was added sodium triacetoxy borohydride (329.46 mg, 1.55 mmol) and acetic acid (161.79 μL, 2.83 mmol). The mixture was stirred for 2 h at 23° C., before being diluted with EtOAc/THF 2:1. The mixture was washed with saturated aqueous NaHCO₃, water, and brine. The organic layer was dried over Na₂SO₄, filtered, and evaporated. Purification by FC (SiO₂; DCM/MeOH) gave the title compound (223 mg, 40.5% yield) as a colorless oil. MS (ESI): m/z=371.2 [M+H]⁺

### Example B.275

### 2-(4-fluorophenyl)-2-(4-piperidyl)acetamide; 4-methylbenzenesulfonic Acid

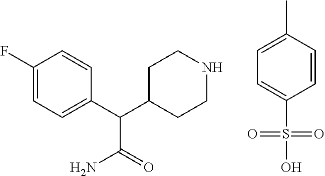 [SMILES: NC(=O)C(c1ccc(F)cc1)C1CCNCC1 ; Cc1ccc(S(=O)(=O)O)cc1]

PTSA (267.17 mg. 1.4 mmol) was added to a stirred solution of tert-butyl 4-[2-amino-1-(4-fluorophenyl)-2-oxo-ethyl]piperidine-1-carboxylate (450.0 mg, 1.34 mmol) in EtOAc (40 mL). The mixture was heated for 16 h at 70° C., before being cooled down and evaporated. Trituration with MTBE gave the title compound (340.2 mg, 61.57% yield) as a light brown solid. MS (ESI): m/z=237.2 [M+H]⁺

### Step a): Tert-Butyl 4-[cyano-(4-fluorophenyl)methylene]piperidine-1-carboxylate

To the solution of 2-(4-fluorophenyl)acetonitrile (CAS RN: 459-22-3; 888 μL, 7.4 mmol) in EtOH (37 mL) was added sodium ethanolate (604 mg, 8.88 mmol), at 23° C. The mixture was stirred for 30 min at this temperature, before being treated dropwise with a solution of tert-butyl 4-oxopiperidine-1-carboxylate (CAS RN: 79099-07-3; 1.47 g, 7.4 mmol) in EtOH (37 mL). The mixture was stirred for 18 h at 23° C., before being poured into a saturated aqueous NH₄Cl solution and EtOAc. The layers were separated, and the aqueous layer was extracted with EtOAc (2×). The combined organic layers were dried over MgSO₄, filtered, and evaporated. Purification by FC (SiO₂; heptane/EtOAc) gave the title compound (2.32 g, 99.1% yield) as a colorless solid. MS (ESI): m/z=261.2 [M-C₄H₈+H]⁺

### Step b): Tert-Butyl 4-[cyano-(4-fluorophenyl)methyl]piperidine-1-carboxylate

A solution of tert-butyl 4-(cyano(4-fluorophenyl)methylene)piperidine-1-carboxylate (526 mg, 1.66 mmol) in 1:1 MeOH/EtOAc (10 mL) was treated with Pd/C 10% (106.2 mg, 100 μmol), at 23° C. The mixture was stinted for 18 h under hydrogen atmosphere at 1.3 bars, before being filtered. The filtrate was evaporated. Purification by FC (SiO₂; heptane/EtOAc) gave the title compound (0.415 g; 78.4% yield) as a colorless gum. MS (ESI): m/z=263.2 [M+H]⁺

### Step c): 2-(4 fluorophenyl)-2-(4 piperidyl)acetic Acid

A solution of tert-butyl 4-(cyano(4-fluorophenyl)methyl)piperidine-1-carboxylate (555 mg, 1.74 mmol) in HBr 48% in water (5.55 mL, 49.1 mmol) was stirred at reflux for 4.5 h, before being evaporated. The residue was suspended in 2-propanol (2 mL), homogenized and filtered. The filter cake was washed three times with 2-propanol (3×1 mL). The mother liquor was completely evaporated and dried for 2 h at high vacuum in the presence of P₂O₅ to yield the title compound (0.535 g; 96.5% yield) as a light brown solid. MS (ESI): m/z=238.2 [M−HBr+H]⁺

### Step d): 2-(1-tert-butoxycarbonyl-4 piperidyl)-2-(4-fluorophenyl)acetic Acid

To a turbid solution of 2-(4-fluorophenyl)-2-(piperidin-4-yl)acetic acid hydrobromide (535 mg, 1.55 mmol) in 1 M NaOH (3.09 mL, 3.09 mmol) was added dropwise a solution of Boc₂O (391 μL, 1.68 mmol) in DME (5 mL). The mixture was stirred for 3 h at 23° C., before being evaporated. The residue was taken up in 1.2 mL citric acid 10% in water (pH approx. 4) and ethyl acetate, and the layers were separated. The aqueous layer was extracted once with ethyl acetate. The combined organic layers were dried over MgSO₄, filtered and evaporated, to give the title compound (0.520 g; 99.6% yield) as a light brown solid. MS (ESI): m/z=336.3 [M−H]⁻

### Step e): Tert-Butyl 4-[2-amino-1-(4-fluorophenyl)-2-oxo-ethyl]piperidine-1-carboxylate

A solution of 2-(l-tert-butoxycarbonyl-4-piperidyl)-2-(4-fluorophenyl)acetic acid (2.0 g, 5.93 mmol) in THE (20 mL) was treated with CDI (1.44 g, 8.89 mmol), at 23° C. The mixture was stirred for 30 min at that temperature, before being treated dropwise with ammonia (25% in water) (1.01 g, 59.28 mmol). The mixture was stirred for another 16 h, before being diluted with EtOAc (40 mL) and washed with water. The organic layer was dried over Na₂SO₄, filtered, and evaporated, to give the title compound (1.9 g, 90.52% yield) as a white solid. MS (ESI): m/z=237.2 [M-Boc+H]⁺

### Example B.277

### 5-[1-[4-(azetidin-3-yl)phenyl]cyclopropyl]-2-tert-butyl-tetrazole; 4-methylbenzenesulfonic Acid

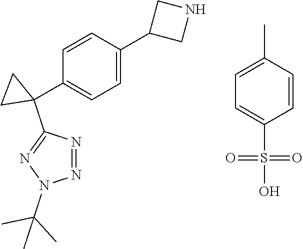 [SMILES: CC(C)(C)n1nnc(C2(c3ccc(C4CNC4)cc3)CC2)n1 ; Cc1ccc(S(=O)(=O)O)cc1]

To a solution of tert-butyl 3-[4-[1-(2H-tetrazol-5-yl)cyclopropyl]phenyl]azetidine-1-carboxylate (1900.0 mg, 5.57 mmol) (80% purity) in EtOAc (30 mL) was added p-toluenesulfonic acid (1149.98 mg, 6.68 mmol). The mixture was stirred for 12 h at 50° C., before being evaporated. Purification by RP-HPLC gave the title compound as an unexpected side product during this deprotection reaction (20.9 mg, 0.56% yield). MS (ESI): m/z=298.2 [M+H]⁺

### Step a): Tert-Butyl 3-[4-(1-cyanocyclopropyl)phenyl]azetidine-1-carboxylate

A vial was charged with tert-butyl 3-bromoazetidine-1-carboxylate (CAS RN: 1064194-10-0; 2.76 g, 11.71 mmol), 1-(4-bromophenyl)cyclopropanecarbonitrile (CAS RN: 124276-67-1; 2.0 g, 9.01 mmol), Ir[dF(CF₃)ppy]₂(dtbbpy)PF₆ (100.95 mg, 0.090 mmol), NiCl₂-dtbbpy (17.92 mg, 0.050 mmol), Na₂CO₃ (1909.04 mg, 18.01 mmol), bis(trimethylsilyl)silyl-trimethyl-silane (2.24 g, 9.01 mmol) and DCE (40 mL). The vial was sealed and placed under nitrogen. The reaction was stirred and irradiated with a 34 W blue LED lamp (7 cm away) equipped with cooling fan to keep the reaction temperature at 25° C. for 20 h. The mixture was filtered off and evaporated. Purification by RP-HPLC gave the title compound (1.60 g, 59.54% yield) as a yellow oil. MS (ESI): m/z=243.4 [M-C₄H₈+H]⁺

### Step b): Tert-Butyl 3-[4-[1-(2H-tetrazol-5-yl)cyclopropyl]phenyl]azetidine-1-carboxylate

Caution, for this reaction, use a protective shield and carry out all operations in the ventilation hood. To a stirred suspension of tert-butyl 3-[4-(1-cyanocyclopropyl)phenyl]azetidine-1-carboxylate (1.70 g, 5.7 mmol) and dibutyloxostannane (425.5 mg, 1.71 mmol) in dry 1,4-dioxane (30 mL) was added azidotrimethylsilane (3.02 mL, 22.79 mmol), at 23° C. The mixture was heated to 110° C., and stirred for 18 h at this temperature. The mixture was cooled down and evaporated, to give the title compound (1.9 g, 84.98% yield) as a dark brown oil. MS (ESI): m/z=340.2 [M−H]⁻

### Example B.278

### 4-methylbenzenesulfonic acid; 3-[4-(2,2,2-trifluoro-1,1-dimethyl-ethoxy)phenyl]azetidine

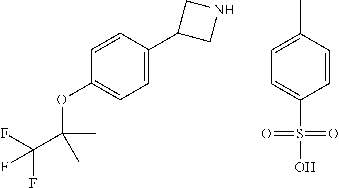 [SMILES: CC(C)(Oc1ccc(C2CNC2)cc1)C(F)(F)F ; Cc1ccc(S(=O)(=O)O)cc1]

A solution of tert-butyl 3-(4-((1,1,1-trifluoro-2-methylpropan-2-yl)oxy)phenyl)azetidine-1-carboxylate (450 mg, 1.25 mmol) and 4-methylbenzenesulfonic acid hydrate (250 mg, 1.31 mmol) in EtOAc (3 mL) was stirred at reflux for 1.5 h, before being cooled down. The precipitate was filtered off, and the cake was washed with EtOAc and dried, to give the title compound (0.436 g; 75.1% yield) as a colorless solid. MS (ESI): m/z=260.2 [M+H]⁺

### Step a): 1-bromo-4-(2,2,2-trifluoro-1,1-dimethyl-ethoxy)benzene

To a solution of 4-((1,1,1-trifluoro-2-methylpropan-2-yl)oxy) aniline (CAS RN: 1255649-29-6; 3.00 g, 13.7 mmol) in ACN (66 mL) was added copper (II) bromide (3.36 g, 15.1 mmol), at 23° C. To the dark suspension was added dropwise a solution of tert-butyl nitrite (1.99 mL, 15.1 mmol) in ACN (16.7 mL) over 30 min. The mixture was stirred for 18 h at 23° C., before being diluted with ACN, treated with silica, and evaporated. Purification by FC (SiO₂; heptane) gave the title compound (1.7 g; 35.5% yield) as a colorless oil. MS (ESI): m/z=282.4 [M+H]⁺

### Step b): Tert-Butyl 3-[4-(2,2,2-trifluoro-1,1-dimethyl-ethoxy)phenyl]azetidine-1-carboxylate

To an 10 mL flask equipped with a magnetic stir bar was added photocatalyst (Ir[dF(CF₃)ppy]₂(dtbpy))PF₆ (31.3 mg, 27.9 μmol), 1-bromo-4-((1,1,1-trifluoro-2-methylpropan-2-yl)oxy)benzene (790 mg. 2.79 mmol), tert-butyl 3-bromoazetidine-1-carboxylate (CAS RN: 1064194-10-0; 659 mg, 2.79 mmol), 1,1,1,3,3,3-hexamethyl-2-(trimethylsilyl)trisilane (862 μL, 2.79 mmol) and anhydrous sodium carbonate (592 mg, 5.58 mmol). The vial was sealed and placed under Ar before DME (11.1 ml) was added. To a separate vial was added nickel(II) chloride ethylane glycol dimethyl ether complex (12.3 mg, 55.8 μmol) and 4,4′-di-tert-butyl-2,2′-bipyridine (15 mg, 55.8 μmol). The precatalyst vial was sealed, purged with Ar, and DME (4 mL) was added. This vial was sonicated for 5 min, after which 1 mL of it was added into the reaction vessel. The mixture was stirred and irradiated with a 420 nm lamp for 65 h, before being filtered. The filtrate was treated with silica gel and evaporated. Purification by FC (SiO₂; heptane/EtOAc) gave the title compound (0.409 g; 38.7% yield) as a colorless solid. MS (ESI): m/z=304.2 [M−C₄H₈+H]⁺

### Example B.280

### 5-[4-(azetidin-3-yl)phenyl]-1-(2,2-dimethylpropyl)triazole; 4-methylbenzenesulfonic Acid

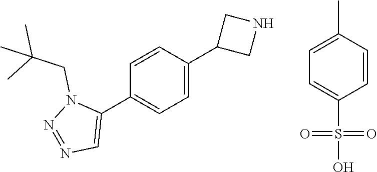 [SMILES: CC(C)(C)Cn1nncc1-c1ccc(C2CNC2)cc1 ; Cc1ccc(S(=O)(=O)O)cc1]

A solution of tert-butyl 3-[4-[3-(2,2-dimethylpropyl)triazol-4-yl]phenyl]azetidine-1-carboxylate (450.0 mg, 1.21 mmol) and p-toluenesulfonic acid monohydrate (346.56 mg, 1.82 mmol) in EtOAc (25 mL) was stirred at 50° C. for 8 h, before being evaporated. The residue was treated with THF (50 mL), and the resulting precipitate was filtered off, washed with THF, and dried, to give the title compound (500.9 mg, 88.5% yield) as a light grey solid. MS (ESI): m/z=271.2 [M+H]⁺

### Step a): 5-(4-bromophenyl)-1-(2,2-dimethylpropyl)triazole

A solution of neopentylamine (CAS RN: 5813-64-9; 5.54 g, 63.4 mmol) in toluene (400 mL) was treated with 4′-bromoacetophenone (CAS RN: 99-90-1; 9.7 g, 48.73 mmol), 1-azido-4-nitro-benzene (CAS RN: 17271-88-4; 8.0 g, 48.73 mmol), 4 Å MS, and acetic acid (877.97 mg, 14.62 mmol), at 23° C. The mixture was refluxed for 72 h, before being cooled down, filtered, and evaporated. Purification by FC (SiO₂; CHCl₃/ACN) gave the title compound (6.65 g, 44.06% yield) as a light brown solid. MS (ESI): m/z=294.2/296.2 [M+H]⁺

### Step b): Tert-Butyl 3-[4-[3-(2,2-dimethylpropyl)triazol-4-yl]phenyl]azetidine-1-carboxylate

To a 40 mL vial equipped with a magnetic stir bar were added tert-butyl 3-bromoazetidine-1-carboxylate (1.0 g, 4.24 mmol), 5-(4-bromophenyl)-1-(2,2-dimethylpropyl)triazole (1.245 g, 4.24 mmol), Ir[dF(CF₃)ppy]₂(dtbbpy)PF₆ (56.97 mg, 0.050 mmol), NiCl₂-glyme (5.58 mg, 0.030 mmol), 4-tert-butyl-2-(4-tert-butyl-2-pyridyl)pyridine (8.18 mg. 0.030 mmol), bis(trimethylsilyl)silyl-trimethyl-silane (1.26 g, 5.08 mmol) and Na₂CO₃ (1.08 g, 10.16 mmol) in DME (40 mL). The vial was sealed and placed under nitrogen. The reaction was stirred and irradiated with a 34 W blue LED lamp (7 cm away) at 25° C. for 14 h, before being filtered and evaporated. Purification by FC (SiO₂; PE/EtOAc) gave the title compound (900 mg, 55.6% yield) as a yellow oil. MS (ESI): m/z=371.4 [M+H]⁺

### Example B.281

### 2-[4-(azetidin-3-yl)phenoxy]-5-(trifluoromethyl)pyrazine; 4-methylbenzenesulfonic Acid

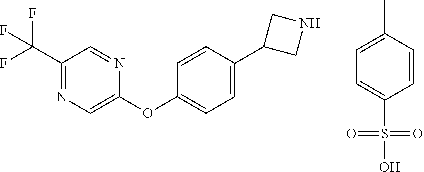 [SMILES: FC(F)(F)c1cnc(Oc2ccc(C3CNC3)cc2)cn1 ; Cc1ccc(S(=O)(=O)O)cc1]

A solution of 4-methylbenzenesulfonic acid hydrate (350.55 mg, 1.84 mmol) and 3-[4-[5-(trifluoromethyl)pyrazin-2-yl]oxyphenyl]azetidine-1-carboxylic acid tert-butyl ester (690 mg, 1.68 mmol) in EtOAc was stirred at reflux for 6 h, before being cooled down. The resulting precipitate was filtered off, washed with EtOAc, and dried, to give the title compound (740 mg, 81.26% yield) as a white solid. MS (ESI): m/z=296.1 [M+H]⁺

### Step a): Tert-Butyl 3-(4-hydroxyphenyl)azetidine-1-carboxylate

To a 40 mL vial equipped with a magnetic stir bar were added tert-butyl 3-bromoazetidine-1-carboxylate (CAS RN: 1064194-10-0; 8.00 g, 33.88 mmol), 4-bromophenol (CAS RN: 106-41-2; 5.862 g, 33.88 mmol), Ir[dF(CF₃)ppy]₂(dtbbpy)PF₆ (379.82 mg, 0.340 mmol), NiCl₂-glyme (37.22 mg, 0.170 mmol), 4-tert-butyl-2-(4-tert-butyl-2-pyridyl)pyridine (54.56 mg, 0.200 mmol), bis(trimethylsilyl)silyl-trimethyl-silane (8425.23 mg, 33.88 mmol) and Na₂CO₃ (7182.41 mg, 67.77 mmol) in DME (60 mL). The vial was sealed and placed under nitrogen. The reaction was stirred and irradiated with a 34 W blue LED lamp (7 cm away), with cooling fan to keep the reaction temperature at 25° C. for 14 hr. The mixture was filtered, evaporated, and purified by RP-HPLC, to give the title compound (6.95 g, 82.28% yield) as a light yellow solid. MS (ESI): m/z=194.0 [M−C₄H₈+H]⁺

### Step b): Tert-Butyl 3-[4-[5-(trifluoromethyl)pyrazin-2 yl]oxyphenyl]azetidine-1-carboxylate

To a solution of 3-(4-hydroxyphenyl)azetidine-1-carboxylic acid tert-butyl ester (450 mg, 1.8 mmol) and potassium carbonate (748.4 mg, 5.41 mmol) in DMSO (6.75 mL) was added 2-bromo-5-(trifluoromethyl)pyrazine (450.66 mg, 1.99 mmol). The mixture was heated to 100° C. and stirred for 1 h at this temperature, before being cooled down and diluted with EtOAc. The organic layer was washed with water and brine, dried over MgSO₄, filtered, and evaporated. Purification by FC (SiO₂; heptane/MTBE) gave the title compound (691 mg, 94.89% yield) as a light yellow solid. MS (ESI): m/z=340.1 [M-tBu+H]⁺

In analogy to Example B.281, the following building block was generated using the relevant commercial building block and 3-(4-hydroxyphenyl)azetidine-1-carboxylic acid tert-butyl ester (described in Step a)) in Step b).

| | | | Building | MS, ESI: |
|---|---|---|---|---|
| Ex. | Structure | Systematic Name | Blocks | m/z |
| B.285 | 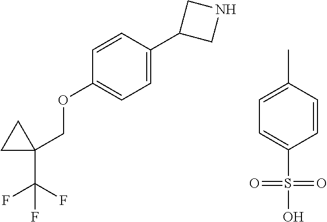 [SMILES: FC(F)(F)C1(COc2ccc(C3CNC3)cc2)CC1 ; Cc1ccc(S(=O)(=O)O)cc1] | 4- methylbenzenesulfonic acid;3-[4-[1- (trifluoromethyl) cyclopropyl]methoxy] phenyl]azetidine | 1- (bromomethyl)- 1- (trifluoromethyl)- cyclopropane (CAS RN: 1155272-93-7) | 272.1 [M + H]⁺ |

### Example B.287

### 3-[4-(azetidin-3-yl)phenyl]-5-[1-(trifluoromethyl)cyclopropyl]-4H-1,2,4-triazole; 4-methylbenzenesulfonic Acid

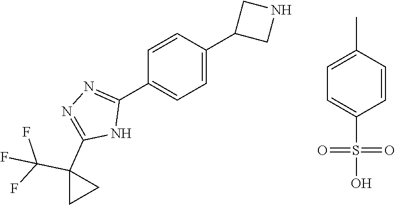 [SMILES: FC(F)(F)C1(c2nnc(-c3ccc(C4CNC4)cc3)[nH]2)CC1 ; Cc1ccc(S(=O)(=O)O)cc1]

To a solution of tert-butyl 3-[4-[5-[1-(trifluoromethyl)cyclopropyl]-4H-1,2,4-triazol-3-yl]phenyl]azetidine-1-carboxylate (700.0 mg, 1.71 mmol) in EtOAc (43.75 mL) was added p-toluenesulfonic acid monohydrate (717.25 mg, 3.77 mmol), at 23° C. The mixture was heated to 47° C. and stirred for 18 h at this temperature, before being evaporated. Trituration with Et₂O gave the title compound (604 mg, 53.99°% yield) as a white solid. MS (ESI): m/z=309.0 [M+H]⁺

### Step a): Tert-Butyl 3-[4-[5-[1-(trifluoromethyl)cyclopropyl]-4H-1,2,4-triazol-3-yl]phenyl]azetidine-1-carboxylate

A solution of 1-(trifluoromethyl)cyclopropanecarbohydrazide (CAS RN: 1016557.86-0; 4.07 g, 24.19 mmol), tert-butyl 3-(4-cyanophenyl)azetidine-1-carboxylate (CAS RN: 206446-41-5; 1.25 g, 4.84 mmol) and potassium carbonate (6.69 g, 48.39 mmol) in 1-butanol (112.5 mL) was stirred for 90 h at 150° C., before being cooled down, filtered, and evaporated. Trituration with hexane gave the title compound (700 mg, 34.71% yield) as a white solid. MS (ESI): m/z=409.0 [M+H]⁺

### Example B.288

### 3-(azetidin-3-yl)-6-(2-chlorophenoxy)pyridazine; 4-methylbenzenesalfonic Acid

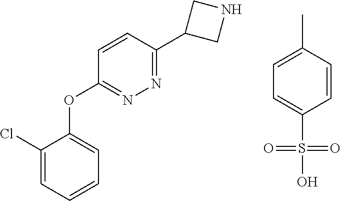 [SMILES: Clc1ccccc1Oc1ccc(C2CNC2)nn1 ; Cc1ccc(S(=O)(=O)O)cc1]

A solution of 3-[6-(2-chlorophenoxy)pyridazin-3-yl]azetidine-1-carboxylic acid tert-butyl ester (150 mg, 0.415 mmol) in EtOAc (5 mL) was treated with PTSA (82.8 mg, 0.435 mmol), at 23° C. The mixture was heated to 70° C. and stirred for 18 h at this temperature, before being cooled down. The mixture was diluted with Et₂O, and the resulting precipitate was filtered off, washed with Et₂O, and dried, to give the title compound (170 mg, 93.56%) as a light brown solid. MS (ESI): m/z=262.1 [M+H]⁺

### Step a): 3-bromo-6-(2-chlorophenoxy)pyridazine

A solution of 3,6-dibromopyridazine (CAS RN: 17973-86-3; 3.57 g, 15 mmol) and 2-chlorophenol (CAS RN: 95-57-8; 2.02 g, 1.61 mL, 15.75 mmol) in DMF (30 mL) was treated with K₂CO₃ (3.73 g, 27 mmol), at 23° C. The mixture was stirred for 4 h at 80° C., before being allowed to cool down. The resulting precipitate was filtered off and washed with DMF. The filtrate was evaporated and purified by FC (SiO₂; heptane/MTBE), to give the title compound (4 g, 88.73%) as a white solid. MS (ESI): m/z=287.0 [M+H]

### Step b): Tert-Butyl 3-[6-(2-chlorophenoxy)pyridazin-3-yl]azetidine-1-carboxylate

To a 20 mL vial equipped with a magnetic stir bar were added 3-bromo-6-(2-chlorophenoxy)pyridazine (1.22 g, 4.27 mmol), tert-butyl 3-bromoazetidine-1-carboxylate (1.01 g, 4.27 mmol), (Ir[dF(CF₃)ppy]₂(dtbpy))PF₆ (47.9 mg, 42.7 μmot), tris(trimethylsilyl)silane (1.32 mL, 4.27 mmol) and anhydrous sodium carbonate (679 mg, 6.41 mmol), under Ar. DME (11.5 mL) was added and the mixture was stirred for 5 min with Ar bubbling through the mixture. The vial was sealed. To a separate vial were added Nickel(II) chloride ethylene glycol dimethyl ether complex (4.69 mg, 21.4 μmol) and 4,4′-di-tert-butyl-2,2′-bipyridine (5.73 mg, 21.4 μmot). DME (1.15 mL) was added. The precatalyst vial was sealed, purged with Ar, and sonicated for 5 min, before being added to the other vial. The mixture was stirred and irradiated with a 420 nm lamp, under Ar atmosphere for 8 h, before being filtered off and evaporated. Purification by FC (SiO₂; heptane/MTBE) gave the title compound (435 mg, 25.8%) as a light brown waxy solid. MS (ESI): m/z=306.1 [M-tBu+H]⁺

### Example B.289

### 5-[1-[4-(azetidin-3-yl)phenyl]cyclopropyl]-1H-tetrazole; 4-methylbenzenesulfonic Acid

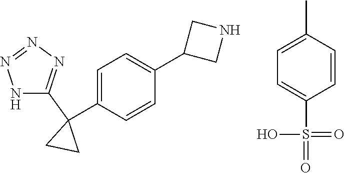 [SMILES: c1cc(C2(c3nnn[nH]3)CC2)ccc1C1CNC1 ; Cc1ccc(S(=O)(=O)O)cc1]

To the solution of tert-butyl 3-[4-[1-(2H-tetrazol-5-yl)cyclopropyl]phenyl]azetidine-1-carboxylate (1.9 g, 4.45 mmol) (80% purity) in EtOAc (30 mL) was added p-toluenesulfonic acid monohydrate (1.0 g, 5.34 mmol). The mixture was stirred for 24 h at 50° C., before being evaporated. Purification by RP-HPLC gave the title compound (323 mg, 15.97% yield) as a light yellow viscous oil. MS (ESI): m/z=242.1 [M+H]

### Step a): Tert-Butyl 3-[4-(1-cyanocyclopropyl)phenyl]azetidine-1-carboxylate

A vial was charged with tert-butyl 3-bromoazetidine-1-carboxylate (CAS RN: 1064194-10-0; 2.76 g, 11.71 mmol), 1-(4-bromophenyl)cyclopropanecarbonitrile (CAS RN: 124276-67-1; 2.0 g, 9.01 mmol), Ir[dF(CF₃)ppy]₂(dtbbpy)PF₆ (100.95 mg, 0.090 mmol), NiCl₂·dtbbpy (17.92 mg, 0.050 mmol), Na₂CO₃ (1909.04 mg, 18.01 mmol), bis(trimethylsilyl)silyl-trimethyl-silane (2.24 g, 9.01 mmol) and DCE (40 mL). The vial was sealed and placed under nitrogen. The reaction was stirred and irradiated with a 34 W blue LED lamp (7 cm away) equipped with cooling fan to keep the reaction temperature at 25° C. for 20 h. The mixture was filtered off and evaporated. Purification by RP-HPLC gave the title compound (1.60 g, 59.54% yield) as a yellow oil. MS (ESI): m/z=243.4 [M-C₄H₈+H]⁺

### Step b): Tert-Butyl 3-[4-[1-(2H-tetrazol-5-yl)cyclopropyl]phenyl]azetidine-1-carboxylate

Caution, for this reaction, use a protective shield and carry out all operations in the ventilation hood. To a stirred suspension of tert-butyl 3-[4-(1-cyanocyclopropyl)phenyl]azetidine-1-carboxylate (1.70 g, 5.7 mmol) and dibutyloxostannane (425.5 mg, 1.71 mmol) in dry 1,4-dioxane (30 mL) was added azidotrimethylsilane (3.02 mL, 22.79 mmol), at 23° C. The mixture was heated to 110° C., and stirred for 18 h at this temperature. The mixture was cooled down and evaporated, to give the title compound (1.9 g, 84.98% yield) as a dark brown oil. MS (ESI): m/z=340.2[M−H]⁻

### Example B.291

### 5-(azetidin-3-yl)-N-(4-isopropylphenyl)-N-methyl-pyridin-2-amine; 4-methylbenzenesulfonic Acid

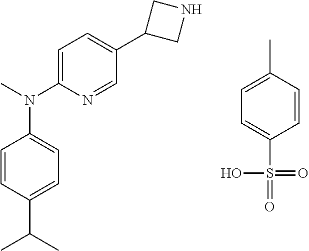 [SMILES: CC(C)c1ccc(N(C)c2ccc(C3CNC3)cn2)cc1 ; Cc1ccc(S(=O)(=O)O)cc1]

A solution of 3-[6-(4-isopropyl-N-methyl-anilino)-3-pyridyl]azetidine-1-carboxylic acid tert-butyl ester (334 mg, 0.875 mmol) and p-toluenesulfonic acid monohydrate (499.59 mg, 2.63 mmol) in EtOAc (3 mL) was heated at reflux for 1 h, before being cooled down to 23° C. and stirred for 18 h at this temperature. The resulting precipitate was filtered off and washed with EtOAc. Purification by FC (Si—NH₂; ACN/MeOH) gave the title compound (0.211 g, 85.6%) as a yellow oil. MS (ESI): m/z=282.3 [M+H]⁺.

### Step a): 5-bromo-N-(4-isopropylphenyl)-N-methyl-pyridin-2-amine

A solution of (5-bromo-2-pyridyl)-p-cumenyl-amine (CAS RN: 107962-10-7; 400 mg, 1.37 mmol) in THE (3.41 mL) was treated with NaH (55% in mineral oil) (71.93 mg, 1.65 mmol), at 0° C. The mixture was stirred for 45 min at this temperature, before being treated with iodomethane (120.25 μL, 1.92 mmol). The mixture was stirred for 46 h at 23° C., before being poured onto water and EtOAc, and the layers were separated. The aqueous layer was extracted with EtOAc. The combined organic layers were dried over MgSO₄, filtered, treated with silica gel, and evaporated. Purification by FC (SiO₂; heptane/EtOAc) gave the title compound (0.363 g; 86.5%) as a colorless oil. MS (ESI): m/z=305.1 [M+H]⁺

### Step b): Tert-Butyl 3-[6-(4-isopropyl-N-methyl-anilino)-3-pyridyl]azetidine-1-carboxylate

To a sealed vial equipped with a stir bar were added bis[3,5-difluoro-2-[5-(trifluoromethyl)-2-pyridyl]phenyl]iridium(1+); 4-tert-butyl-2-(4-tert-butyl-2-pyridyl)pyridine; hexafluorophosphate (13.23 mg, 0.012 mmol), 3-bromoazetidine-1-carboxylic acid tert-butyl ester (417.74 mg, 1.77 mmol), (5-bromo-2-pyridyl)-methyl-p-cumenyl-amine (360 mg, 1.18 mmol), bis(trimethylsilyl)silyl-trimethyl-silane (363.89 μL, 1.18 mmol) and Na₂CO₃ (250.03 mg, 2.36 mmol). The vial was sealed and placed under Ar before ethylene glycol dimethyl ether (3.4 mL) was added. To a separate vial were added dichloronickel; 1,2-dimethoxyethane (2.59 mg, 0.012 mmol) and 4-tert-butyl-2-(4-tert-butyl-2-pyridyl)pyridine (3.17 mg, 0.012 mmol). This vial was sealed, purged with Ar, and ethylene glycol dimethyl ether (1 mL) was added. The mixture was sonicated for 5 min, after which 0.5 mL of it was added to the main reaction mixture. The mixture was stirred and irradiated with a 420 nm lamp (75% intensity) for 6 h, before being filtered off. Purification by FC (SiO₂; heptane/EtOAc) gave the title compound (0.334 g; 74.2%) as a light yellow oil. MS (ESI): m/z=382.3 [M+H]⁺

In analogy to Example B.291, the following building block was generated using the relevant commercial building block in Step a).

| | | | Building | MS, ESI: |
|---|---|---|---|---|
| Ex. | Structure | Systematic Name | Blocks | m/z |
| B.292 | 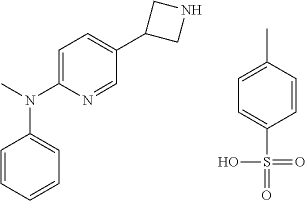 [SMILES: CN(c1ccccc1)c1ccc(C2CNC2)cn1 ; Cc1ccc(S(=O)(=O)O)cc1] | 45-(azetidin-3-yl)-N- methyl-N-phenyl- pyridin-2-amine;4- methylbenzenesulfonic acid | 5-Bromo-N- phenyl-2- pyridinamine (CAS RN: 54904-03-9) | 240.2 [M + H]⁺ |

### Example B.293

### 1-[4-(azetidin-3-yl)phenyl]piperidine-2-carboxamide; 4-methylbenzenesulfonic Acid

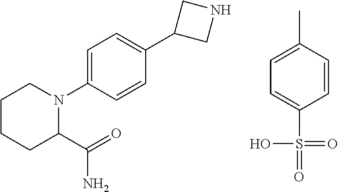 [SMILES: NC(=O)C1CCCCN1c1ccc(C2CNC2)cc1 ; Cc1ccc(S(=O)(=O)O)cc1]

PTSA (635.02 mg, 3.34 mmol) was added to a solution of tert-butyl 3-[4-(2-carbamoyl-1-piperidyl)phenyl]azetidine-1-carboxylate (400.0 mg, 1.11 mmol) in ACN (50 mL). The mixture was refluxed for 6 h, before being cooled down. The precipitate was collected by filtration and recrystallized from i-PrOH, to give the title compound (340 mg, 68.78% yield) as a light brown solid. MS (ESI): m/z=260.0 [M+H]⁺

### Step a): Tert-Butyl 3-(4-bromophenyl)azetidine-1-carboxylate

To a mixture of 4-bromophenylboronic acid (CAS RN: 5467-74-3; 200.04 g, 996.08 mmol) and tert-butyl 3-iodoazetidine-1-carboxylate (CAS RN: 254454-54-1; 141.0 g, 498.04 mmol) in 2-propanol (500 mL) was added rac-(1R,2R)-2-aminocyclohexan-1-ol (3.44 g, 29.88 mmol), and nickel(H) iodide (9.34 g, 29.88 mmol). A mixture of sodium bis(trimethylsilyl)amide in THE (1 L, 1000 mmol) was added slowly to the reaction mixture, under N₂ and keeping the temperature below 30° C. After the resulting mixture was stirred at 25° C. for 30 min, the mixture was heated to 80° C. and stirred for 12 h. The reaction mixture was poured onto H₂O (3 L) and EtOAc(3 L), and the layers were separated. The aqueous layer was extracted twice with EtOAc (2×2 L). The combined organic layers were evaporated, and purified by FC (SiO₂; PE/EtOAc), to give the title compound (140 g, 90.04% yield) as an off-white oil. MS (ESI): m/z=256.1 [M-tBu+H]⁺

### Step b): 1-[4-(1-tert-butoxycarbonylazetidin-3 yl)phenyl]piperidine-2-carboxylic Acid

A solution of pipecolinic acid (2.07 g, 16.02 mmol), tert-butyl 3-(4-bromophenyl)azetidine-1-carboxylate (2.0 g, 6.41 mmol), copper(I) iodide (0.04 mL, 1.28 mmol) and phosphoric acid, potassium salt (2.12 mL, 25.62 mmol) in DMSO (40 mL) was stirred for 24 h at 110° C., under Ar. The mixture was cooled down, poured onto water, and acidified with an aqueous solution of citric acid. The aqueous mixture was extracted with EtOAc, and the combined organic layers were washed with brine, dried over Na₂SO₄, filtered, and evaporated, to give the title compound (1.9 g, 82.28% yield) as a white solid. MS (ESI): m/z=361.0 [M+H]⁺

### Step c): Tert-Butyl 3-[4-(2-carbamoyl-1-piperidyl)phenyl]azetidine-1-carboxylate

A solution of 1-[4-(1-tert-butoxycarbonylazetidin-3-yl)phenyl]piperidine-2-carboxylic acid (400.0 mg, 1.11 mmol) and CDI (233.92 mg, 1.44 mmol) in THE (25 mL) was stirred at 50° C. for 1 h, before being cooled down to 23° C. The mixture was treated with ammonia (188.99 mg, 11.1 mmol) (28% in water) and stirred for 12 h at 23° C., before being evaporated. The residue was dissolved in water, and the resulting precipitate was filtered and dried, to give the title compound (400 mg, 95.3% yield) as a light brown solid. MS (ESI): m/z=360.2 [M+H]⁺

In analogy to Example B.293, the following building block was generated using the relevant commercial building block in Step b).

| | | | Building | MS, ESI: |
|---|---|---|---|---|
| Ex. | Structure | Systematic Name | Blocks | m/z |
| B.294 | 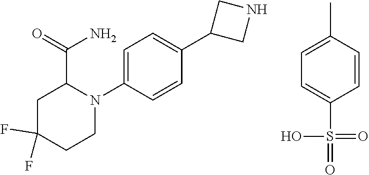 [SMILES: NC(=O)C1CC(F)(F)CCN1c1ccc(C2CNC2)cc1 ; Cc1ccc(S(=O)(=O)O)cc1] | 1-[4-(azetidin-3- yl)phenyl]-4,4- difluoro-piperidine-2- carboxamide;4- methylbenzenesulfonic acid | 4,4- difluoropiperidine- 2- carboxylic acid (CAS RN: 1402664-50-9) | 296.2 [M + H]⁺ |
| B.295 | 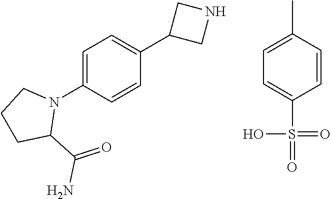 [SMILES: NC(=O)C1CCCN1c1ccc(C2CNC2)cc1 ; Cc1ccc(S(=O)(=O)O)cc1] | 1-[4-(azetidin-3- yl)phenyl]pyrrolidine- 2-carboxamide;4- methylbenzenesulfonic acid | Pyrrolidine-2- carboxylic acid (CAS RN: 609-36-9) | 246.2 [M + H]⁺ |

### Example E.1

### 6-[(4-dimethylphosphorylphenyl)methyl]-2-azaspiro[3.3]heptane; 4-methylbenzenesulfonic Acid

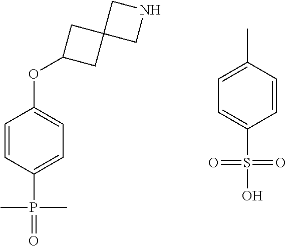 [SMILES: CP(C)(=O)c1ccc(OC2CC3(CNC3)C2)cc1 ; Cc1ccc(S(=O)(=O)O)cc1]

To a solution of tert-butyl 6-[(4-dimethylphosphorylphenyl)methyl]-2-azaspiro[3.3]heptane-2-carboxylate (1.35 g, 3.71 mmol) in EtOAc (50 mL), p-toluenesulfonic acid monohydrate (1.41 g, 7.43 mmol) was added. The mixture was stirred for 12 h at 25° C., before being evaporated to dryness. The residue was purified by RP-HPLC, to give the title compound (474.3 mg, 27.85% yield) as a light yellow solid. MS (ESI): m/z=264.2 [M+H]⁺

### Step a) Tert-Butyl 6-[(4,4,5,5-tetramethyl-1,3,2-dioxaborolan-2-yl)methylene]-2-azaspiro[3.3]heptane-2-carboxylate

A mixture of 2,2,6,6-tetramethylpiperidine (95.9 mL, 568 mmol) in THE (750 mL) was cooled to −30° C. under a N₂ atmosphere. n-BuLi (227 mL, 568 mmol) was added dropwise, and the reaction mixture was stirred at the same temperature for 30 min. Next, the reaction was cooled to −60° C., and a solution of 4,4,5,5-tetramethyl-2-[(4,4,5,5-tetramethyl-1,3,2-dioxaborolan-2-yl)methyl]-1,3,2-dioxaborolane (136 g, 506 mmol) in THE (750 mL) was added dropwise. After stirring for 30 min, a solution of tert-butyl 6-oxo-2-azaspiro[3.3]heptane-2-carboxylate (100 g, 473 mmol) in THE (300 mL) was added in dropwise at −60° C. The reaction mixture was allowed to slowly warm up to 25° C. and stirred at 25° C. for 12 h. The mixture was added H₂O (80 mL) slowly and then purified together with an additional batch of equal size by silica gel column (PE/EA=1:0 to 3:1 gradient) to give the title compound (220 g, 656 mmol, approx 69% yield per batch) as a white solid which was confirmed by ¹H NMR (400 MHz, CHLOROFORM-d) δ=5.21-5.16 (m, 1H), 3.99-3.89 (m, 4H), 3.13-2.90 (m, 4H), 1.46-1.41 (m, 9H), 1.26-1.20 ppm (m, 13H).

### Step b) Tert-Butyl 6-[(4-dimethylphosphorylphenyl)methylene]-2-azaspiro[3.3]heptane-2-carboxylate

To a stirred suspension of 1-bromo-4-dimethylphosphoryl-benzene (1.75 g, 7.52 mmol), tert-butyl 6-[(4,4,5,5-tetramethyl-1,3,2-dioxaborolan-2-yl)methylene]-2-azaspiro[3.3]heptane-2-carboxylate (2.52 g, 7.52 mmol) and potassium carbonate (2.08 g, 15.03 mmol) in 1,4-Dioxane (59.5 mL) and Water (10.5 mL), flushed with Argon for 5 minutes, Pd(dppf)Cl₂·CH₂Cl₂ (1043.53 mg, 1.28 mmol) was added. The mixture was stirred for 18 h at 80° C. in Argon atmosphere (sealed tube). After cooling to RT, the reaction mixture was filtered through SiO₂ (10 g) and washed with 1,4-dioxane (50 mL). The filtrate was concentrated to give crude product which was purified by FC (SiO₂, PE/MTBE then MTBE/MeOH) to obtain the title compound (1.40 g, 46.9% yield) as a grey solid. MS (ESI): m/z=362.2 [M+H]⁺

### Step c) Tert-Butyl 6-[(4-dimethylphosphorylphenyl)methyl]-2-azaspiro[3.3]heptane-2-carboxylate

The stirred solution of tert-butyl 6-[(4-dimethylphosphorylphenyl)methylene]-2-azaspiro[3.3]heptane-2-carboxylate (1.40 g, 3.87 mmol) and Pd/C (10%) (140 mg) in EtOAc (100 mL) was hydrogenated at 3800 mmHg for 18 h at 25° C. The reaction mixture was filtered and concentrated in vacuum to give the crude title compound (1.35 g, 79.59% yield) as a light green solid. MS (ESI): m/z=364.4 [M+H]⁺

### Example E.2

### 6-[(5-dimethylphosphoryl-2-pyridyl)methyl]-2-azaspiro[3.3]heptane; 4-methylbenzenesulfonic Acid

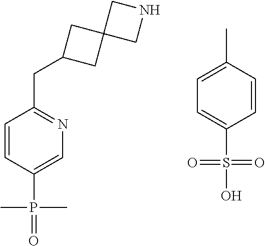 [SMILES: CP(C)(=O)c1ccc(CC2CC3(CNC3)C2)nc1 ; Cc1ccc(S(=O)(=O)O)cc1]

A solution of tert-butyl 6-[(5-dimethylphosphoryl-2-pyridyl)methyl]-2-azaspiro[3.3]heptane-2-carboxylate (998.0 mg, 2.74 mmol) and p-toluenesulfonic acid monohydrate (1.30 g, 6.85 mmol) in EtOAc (70 mL) was stirred at 25° C. for 18 h, before being evaporated. Trituration with MTBE and Et₂O gave the title compound (781.0 mg, 44.51% yield) as a light brown waxy solid. MS (ESI): m/z=265.2 [M+H]⁺

### Step a) Tert-Butyl 6-[(4,4,5,5-tetramethyl-1,3,2-dioxaborolan-2-yl)methylene]-2-azaspiro[3.3]heptane-2-carboxylate

A mixture of 2,2,6,6-tetramethylpiperidine (95.9 mL, 568 mmol) in THF (750 mL) was cooled to −30° C. under a N₂ atmosphere. n-BuLi (227 mL, 568 mmol) was added dropwise, and the reaction mixture was stirred at the same temperature for 30 min. Next, the reaction was cooled to −60° C., and a solution of 4,4,5,5-tetramethyl-2-[(4,4,5,5-tetramethyl-1,3,2-dioxaborolan-2-yl)methyl]-1,3,2-dioxaborolane (136 g, 506 mmol) in THF (750 mL) was added dropwise. After stirring for 30 min, a solution of tert-butyl 6-oxo-2-azaspiro[3.3]heptane-2-carboxylate (100 g, 473 mmol) in THF (300 mL) was added in dropwise at −60° C. The reaction mixture was allowed to slowly warm up to 25° C. and stirred at 25° C. for 12 h. The mixture was added H₂O (80 mL) slowly and then purified together with an additional batch of equal size by silica gel column (PE/EA=1:0 to 3:1 gradient) to give the title compound (220 g, 656 mmol, approx 69% yield per batch) as a white solid which was confirmed by ¹H NMR (400 MHz, CHLOROFORM-d) δ=5.21-5.16 (m, 1H), 3.99-3.89 (m, 4H), 3.13-2.90 (m, 4H), 1.46-1.41 (m, 9H), 1.26-1.20 ppm (m, 13H).

### Step b) Tert-Butyl 6-[(5-dimethylphosphoryl-2-pyridyl)methylene]-2-azaspiro[3.3]heptane-2-carboxylate

2-Chloro-5-dimethylphosphoryl-pyridine (1.02 g, 5.37 mmol), tert-butyl 6-[(4,4,5,5-tetramethyl-1,3,2-dioxaborolan-2-yl)methylene]-2-azaspiro[3.3]heptane-2-carboxylate (1.80 g, 5.37 mmol), Pd(dppf)Cl₂·CH₂Cl₂ (657.69 mg, 0.81 mmol) and potassium carbonate (1.48 g, 10.74 mmol) were dissolved in 1,4-Dioxane (50 mL) and Water (10 mL). The reaction mixture was stirred at 88° C. under Argon for 8 h (sealed tube). The reaction mixture was evaporated. The residue was partitioned between ethyl acetate and water. The organic layer was washed with brine. The organic layer was dried over sodium sulfate, filtered and evaporated. Purification by FC (SiO₂; PE/MTBE) gave the title compound (1.10 g, 51.44% yield) as a yellow solid. MS (ESI): m/z=363.2 [M+H]⁺

### Step c) Tert-Butyl 6-[(5-dimethylphosphoryl-2-pyridyl)methyl]-2-azaspiro[3.3]heptane-2-carboxylate

A solution of tert-butyl 6-[(5-dimethylphosphoryl-2-pyridyl)methylene]-2-azaspiro[3.3]heptane-2-carboxylate (1.10 g, 3.04 mmol) and Pd/C (10%) (110 mg) in EtOAc (100 mL) was hydrogenated at 3800 mmHg for 48 h at RI (LCMS control). The reaction mixture was filtered off and evaporated, to give the crude title compound (998.0 mg, 85.71% yield) as a light grey solid. MS (ESI): m/z=365.2 [M+H]⁺

### Example E.8

### 1-[3-(azetidin-3-yl)-1-bicyclo[1.1.1]pentanyl]-5-cyclopropyl-3-methyl-pyrazole; 4-methylbenzenesulfonic Acid

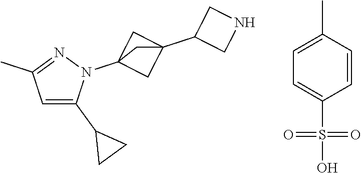 [SMILES: Cc1cc(C2CC2)n(C23CC(C4CNC4)(C2)C3)n1 ; Cc1ccc(S(=O)(=O)O)cc1]

To a solution of tert-butyl 3-[3-(5-cyclopropyl-3-methyl-pyrazol-1-yl)-1-bicyclo[1.1.1]pentanyl]azetidine-1-carboxylate (558.0 mg, 1.62 mmol) in MeOH (3 mL), p-toluenesulfonic acid (419.65 mg, 2.44 mmol) was added, and the resulting mixture was stirred for 16 h. The mixture was evaporated, triturated with acetonitrile (10 mL), filtered and dried. Purification by RP-HPLC gave the title compound (390.0 mg, 54.88% yield) as a white solid. MS (ESI): m/z=244.0 [M+H]⁺

### Step a) Tert-Butyl 3-[3-(benzyloxycarbonylamino)-1-bicyclo[1.1.1]pentanyl]azetidine-1-carboxylate

To a solution of 3-(1-tert-butoxycarbonylazetidin-3-yl)bicyclo[1.1.1]pentane-1-carboxylic acid (4.25 g, 15.9 mmol) and benzyl alcohol (3.29 mL, 31.8 mmol) in Toluene (60 mL) at ambient temperature was added triethylamine (6.65 mL, 47.7 mmol). The mixture was stirred for 5 min, and diphenylphosphonic azide (3.6 mL, 16.69 mmol) was added. The mixture was stirred another 15 min at ambient temperature, and for 16 h at 100° C. After cooling, the mixture was poured into ice-cold water (100 mL) and extracted with MTBE. The organic layer was washed with H₂O and brine, dried over Na₂SO₄, filtered, and evaporated. Purification by FC gave the title compound (3.7 g, 59.36% yield) as a white solid. MS (ESI): m/z=371.2 [M−H]⁻

### Step b): Tert-Butyl 3-(3-amino-1-bicyclo[1.1.1]pentanyl)azetidine-1-carboxylate

To a solution of tert-butyl 3-[3-(benzyloxycarbonylamino)-1-bicyclo[1.1.1]pentanyl]azetidine-1-carboxylate (4.15 g, 11.14 mmol) in MeOH (50 mL) was added Pd/C (10%) (0.58 mL, 0.56 mmol). The reaction mixture was stirred for 48 h at room temperature under hydrogen atmosphere. The solids were removed by filtration and the filtrate was concentrated in vacuo, to give the title compound (2.6 g, 93.01% yield) as a colorless oil. MS (ESI): m/z=239.2 [M+H]⁺

### Step c): Tert-Butyl 3-[3-(5-cyclopropyl-3-methyl-pyrazol-1 yl)-1-bicyclo[1.1.1]pentanyl]azetidine-1-carboxylate

To a solution of tert-butyl 3-(3-amino-1-bicyclo[1.1.1]pentanyl)azetidine-1-carboxylate (1.3 g, 5.45 mmol) in DMF (10 mL) 1-cyclopropylbutane-1,3-dione (0.76 g, 6.0 mmol) and O-(4-nitrobenzoyl)hydroxylamine (1.49 g, 8.18 mmol) were added. The mixture was stirred at 85° C. for 2 h, cooled down, water (25 mL) was added and extracted with at EtOAc (3 times 15 mL each). The organics were then separated and dried (Na₂SO₄) before concentration to dryness. Purification by RP-HPLC gave the title compound (558.0 mg, 29.78% yield). MS (ESI): m/z=344.2 [M+H]⁺

### Example B.311

### 6-[[5-(trifluoromethyl)pyrazol-1-yl]methyl]-2-azaspiro[3.3]heptane; 4-methylbenzenesulfonic Acid

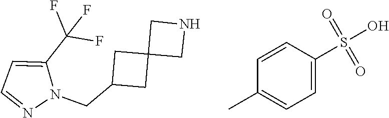 [SMILES: FC(F)(F)c1ccnn1CC1CC2(CNC2)C1 ; Cc1ccc(S(=O)(=O)O)cc1]

To a solution of tert-butyl 6-[[5-(trifluoromethyl)pyrazol-1-yl]methyl]-2-azaspiro[3.3]heptane-2-carboxylate (200 mg, 0.58 mmol) in EtOAc (2 mL) was added p-toluenesulfonic acid (110 mg, 0.64 mmol) and stirred for 12 h at 80° C. The reaction mixture was filtered and concentrated under reduced pressure to give the title compound (230 mg, 0.55 mmol, 95% yield) as a yellow oil. MS (ESI): m/z=246.1 [M+H]⁺.

### Step a) tert-butyl 6-[[5-(trifluoromethyl)pyrazol-1-yl]methyl]-2-azaspiro[3.3]heptane-2-carboxylate

To the mixture of tert-butyl 6-(hydroxymethyl)-2-azaspiro[3.3]heptane-2-carboxylate (CAS: 1363381-93-4) (10.0 g, 44.0 mmol), 5-(trifluoromethyl)-1H-pyrazole (5.99 g, 44.0 mmol), triphenylphosphine (14.4 g, 55.0 mmol) in THE (100 mL) was added diisopropyl azodicarboxylate (10.4 mL, 52.8 mmol) at 0° C., then the reaction mixture was stirred at 20° C. for 12 h under N₂. The reaction mixture was diluted with water 100 mL and extracted with EtOAc 300 mL (100 mL×3). The combined organic layers were washed with aq. sat. NaCl solution (50 mL), dried over Na₂SO₄, filtered and concentrated under reduced pressure to give a residue. The residue was purified by normal phase preparative HPLC to give the title compound (3.7 g, 10.7 mmol, 24.4% yield) as a yellow solid. MS (ESI): m/z=346.1 [M+H]⁺

In analogy to Example B.311, the following building block was generated using the relevant commercial building block in Step a).

| | | | Building | MS, ESI: |
|---|---|---|---|---|
| Ex. | Structure | Systematic Name | Blocks | m/z |
| P.51 | 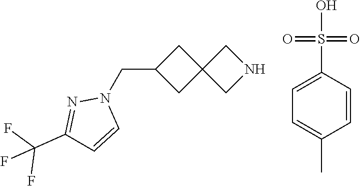 [SMILES: FC(F)(F)c1ccn(CC2CC3(CNC3)C2)n1 ; Cc1ccc(S(=O)(=O)O)cc1] | 6-[[3- (trifluoromethyl) pyrazol- 1-yl]methyl]-2- azaspiro[3.3]heptane; 4- methylbenzenesulfonic acid | 5- (trifluoromethyl)- 1H-pyrazole | 246.2 [M − TsOH + H]⁺ |
| | (regioisomeric product | | | |
| | generated together with | | | |
| | B.311) | | | |
| B.357 | 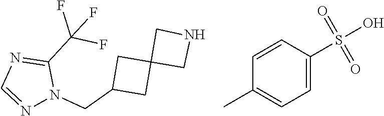 [SMILES: FC(F)(F)c1ncnn1CC1CC2(CNC2)C1 ; Cc1ccc(S(=O)(=O)O)cc1] | 6-[[5- (trifluoromethyl)- 1,2,4-triazol-1- yl]methyl]-2- azaspiro[3.3]heptane; 4- methylbenzenesulfonic acid | 3- (trifluoromethyl)- 1H-1,2,4-triazole | 247.2 [M − TsOH + H]⁺ |

### Example B.317

### 2-(2-azaspiro[3.3]heptan-6-ylmethyl)-5-[1-(trifluoromethyl)cyclopropyl]-1,3,4-oxadiazole; 4-methylbenzenesulfonic Acid

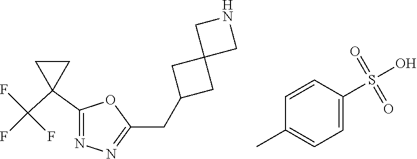 [SMILES: FC(F)(F)C1(c2nnc(CC3CC4(CNC4)C3)o2)CC1 ; Cc1ccc(S(=O)(=O)O)cc1]

To a solution of 6-[[5-[1-(trifluoromethyl)cyclopropyl]-1,3,4-oxadiazol-2-yl]methyl]-2-azaspiro[3.3]heptane-2-carboxylic acid tert-butyl ester (3.22 g, 8.31 mmol) in isopropyl acetate (163 mL) was added p-toluenesulfonic acid monohydrate (1.9 g, 9.97 mmol). The mixture was stirred at 85° C. for 4 h. The reaction mixture was concentrated in vacuo. Et₂O (100 mL) was added (starts to partly crystallize overnight). The mixture was concentrated in vacuo to afford the title compound (4.25 g, 45%) as a white solid. MS (ESI): m/z=288.0 [M+H]⁺

### Step a) 6-(2-hydrazino-2-keto-ethyl)-2-azaspiro[3.3]heptane-2-carboxylic Acid Tert-Butyl Ester

To a solution of 2-(2-tert-butoxycarbonyl-2-azaspiro[3.3]heptan-6-yl)acetic acid (CAS: 1251002-39-7) (2.5 g, 9.79 mmol) in tetrahydrofuran (30 mL) was added CDI (1.75 g, 10.8 mmol). The mixture was stirred at 70° C. for 1.5 h. The RM was cooled to 0° C., hydrazine monohydrate (1.47 g, 1.42 mL, 29.4 mmol) was added, and the mixture was stirred at RT for 30 min. The reaction mixture was poured into EtOAc/THF 2:1, washed with water and brine, dried over Na₂SO₄ and concentrated in vacuo to afford the title compound (3.85 g, 99%) as a white solid. MS (ESI): m/z=214.1 [M+H-C₄H₈]⁺

### Step b) 6-[2-keto-2-[N′-[1-(trifluoromethyl)cyclopropanecarbonyl]hydrazino]ethyl]-2-azaspiro[3.3]heptane-2-carboxylic Acid Tert-Butyl Ester

To a solution of 1-(trifluoromethyl)cyclopropanecarboxylic acid (CAS: 277756-46-4) (1.5 g, 9.72 mmol) in dichloromethane (30 mL) was added 1-chloro-N,N,2-trimethylpropenylamine (1.56 g, 1.54 mL, 11.7 mmol). The mixture was stirred at RT for 2 h. This acid chloride solution was added was added dropwise to a solution of 6-(2-hydrazino-2-keto-ethyl)-2-azaspiro[3.3]heptane-2-carboxylic acid tert-butyl ester (3.85 g, 9.72 mmol) and DIEA (2.51 g, 3.4 mL, 19.4 mmol) in tetrahydrofuran (20 mL) at room temperature. The reaction mixture was stirred at RT for 2 h. The reaction mixture was poured into EtOAc/THF 2:1 and washed with water and brine. The organic layer was dried over Na₂SO₄ and concentrated in vacuo. The crude material was purified by flash chromatography (silica gel, 80 g, 0% to 5% methanol in CH₂Cl₂) to the title compound (3.49 g, 84.14%) as white solid. MS (ESI): m/z=350.1 [M+H-C₄H₈]⁺

### Step c) 6-[[5-[1-(trifluoromethyl)cyclopropyl]-1,3,4-oxadiazol-2-yl]methyl]-2-azaspiro[3.3]heptane-2-carboxylic Acid Tert-Butyl Ester

To a solution of 6-[2-keto-2-[N′-[1-(trifluoromethyl)cyclopropanecarbonyl]hydrazino]ethyl]-2-azaspiro[3.3]heptane-2-carboxylic acid tert-butyl ester (3.49 g, 8.61 mmol) in tetrahydrofuran (40 mL) was added Burgess Reagent (4.1 g, 17.2 mmol). The mixture was stirred at 60° C. for 1 h. The reaction mixture was poured into EtOAc/THF 3:1 and washed with water, and brine. The organic layer was dried over Na₂SO₄ and concentrated in vacuo. The crude material was purified by flash chromatography (silica gel, 80 g, 0% to 5% methanol in CH₂Cl₂) to afford 6-[[5-[1-(trifluoromethyl)cyclopropyl]-1,3,4-oxadiazol-2-yl]methyl]-2-azaspiro[3.3]heptane-2-carboxylic acid tert-butyl ester (3.22 g, 92%) as white solid. MS (ESI): m/z=388.2 [M+H]⁺.

### Example P.23

### 1-(2-Azaspiro[3.3]heptan-6-ylmethyl)-4-(trifluoromethyl)pyridin-2-one; 4-methylbenzenesulfonic Acid

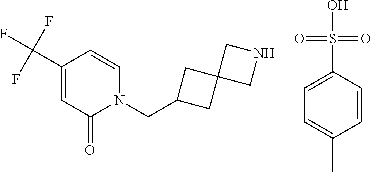

To a solution of tert-butyl 6-[[2-oxo-4-(trifluoromethyl)-1-pyridyl]methyl]-2-azaspiro[3.3]heptane-2-carboxylate (5 g, 13.4 mmol) in ethyl acetate (150 mL) was added p-toluenesulfonic acid monohydrate (5.1 g, 26.8 mmol) and stirred at 25° C. for 72 h. The reaction mixture was filtered and washed with diethyl ether to afford 1-(2-azaspiro[3.3]heptan-6-ylmethyl)-4-(trifluoromethyl)pyridin-2-one; 4-methylbenzenesulfonic acid (5.34 mg, 85% yield) as light yellow solid. MS (ESI): m/z=273.0 [M-TsOH+H]⁺.

### Step a) Tert-Butyl 6-(methylsulfonyl/oxymethyl)-2-azaspiro[3.3]heptane-2-carboxylate

To a stirred solution of tert-butyl 6-(hydroxymethyl)-2-azaspiro[3.3]heptane-2-carboxylate (CAS: 1363381-93-4) (14.9 g, 65.7 mmol) in DCM (299 mL) was added triethylamine (13.7 mL, 98.6 mmol), cooled the reaction mixture to 0° C. followed by dropwise addition of methanesulfonyl chloride (6.1 mL, 78.9 mmol) then reaction mixture was stirred at room temperature for 4 h. The reaction mixture was diluted with water and extracted with DCM, washed with water, brine, dried over anhydrous sodium sulphate and evaporate under reduced pressure to give the title compound (19.8 g, 64.8 mmol, 93.7% yield) as a light yellow solid. MS (ESI): m/z=250.0 [M-Bu+H]⁺.

### Step b) Tert-Butyl 6-[[2-oxo-4-(trifluoromethyl)-1-pyridyl]methyl]-2-azaspiro[3.3]heptane-2-carboxylate

4-(trifluoromethyl)-1H-pyridin-2-one (CAS: 50650-59-4) (5.34 g, 32.8 mmol) was added in small portions under argon at 0° C. to a suspension of sodium hydride 60% in oil (2.14 g, 49.1 mmol) in DMF (100 mL). The mixture was stirred at 0° C. for 10 min and at room temperature for 30 min. The reaction mixture was cooled to 0° C. and ten-butyl 6-(methylsulfonyloxymethyl)-2-azaspiro[3.3]heptane-2-carboxylate (10 g, 32.7 mmol), sodium iodide (4.91 g, 32.7 mmol) were added in one portion. The mixture was stirred at 0° C. for 1 hour then at 80° C. for 18 hours. The reaction mixture was poured into EtOAc and washed with water and brine. The organic layer was dried over Na₂SO₄ and concentrated in vacuo. The crude material was purified by flash chromatography (silica gel, 120 g, 0% to 100% tert-butylmethylether in heptane) to afford the title compound (3.6 g, 29% yield) as yellow solid. MS (ESI): m/z=273.0 [M+H]⁺.

In analogy to Example P.23, the following building block was generated using the relevant commercial building block in Step b). In some cases NMP was used as solvent.

| | | | Building | MS, ESI: |
|---|---|---|---|---|
| Ex. | Structure | Systematic Name | Blocks | m/z |
| P.24 | 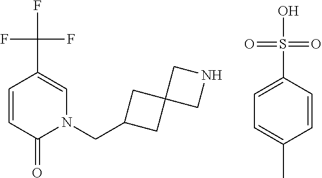 [SMILES: O=c1ccc(C(F)(F)F)cn1CC1CC2(CNC2)C1 ; Cc1ccc(S(=O)(=O)O)cc1] | 1-(2- azaspiro[3.3]heptan-6- ylmethyl)-5- (trifluoromethyl) pyridin-2-one; 4- methylbenzenesulfonic acid | 5- (trifluoromethyl)- 1H-pyridin-2-one (CAS: 33252-63- 0) | 273.0 [M − TsOH + H]⁺ |
| B.419 | 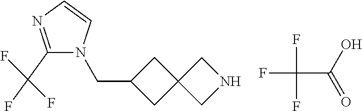 | 6-[[2- (trifluoromethyl) imidazol-1- yl]methyl]-2- azaspiro[3.3]heptane; 2,2,2-trifluoroacetic acid | 2- (trifluoromethyl)- 1H-imidazole (CAS: 66675-22- 7) | 246.2 [M − TFA + H]⁺ |
| B.509 | 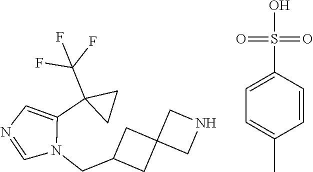 | 6-[[5-[1- (trifluoromethyl) cyclopropyl] imidazol-1- yl]methyl]-2- azaspiro[3.3]heptane; 4- methylbenzenesulfonic acid | 4-[1- (trifluoromethyl) cyclopropyl]-1H- imidazole (C.18) | 286.2 [M − TsOH + H]⁺ |
| | (regioisomer B.538 | | | |
| | generated concurrently) | | | |
| B.510 | 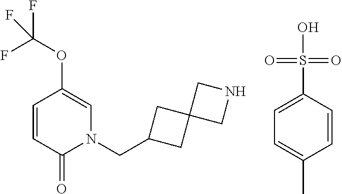 [SMILES: O=c1ccc(OC(F)(F)F)cn1CC1CC2(CNC2)C1 ; Cc1ccc(S(=O)(=O)O)cc1] | 1-(2- azaspiro[3.3]heptan-6- ylmethyl)-5- (trifluoromethoxy) pyridin-2-one; 4- methylbenzenesulfonic acid | 5- (trifluoromethoxy) pyridin-2-ol (CAS: 1052722- 33-4) | 333.2 [M − TsOH + H]⁺ |
| B.511 | 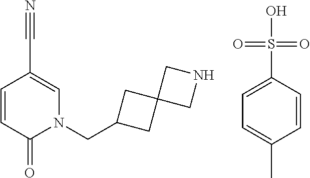 [SMILES: N#Cc1ccc(=O)n(CC2CC3(CNC3)C2)c1 ; Cc1ccc(S(=O)(=O)O)cc1] | 1-(2- azaspiro[3.3]heptan-6- ylmethyl)-6-oxo- pyridine-3- carbonitrile; 4- methylbenzenesulfonic acid | 6- hydroxy- nicotinonitrile (CAS: 95891-30-8) | 230.0 [M − TsOH + H]⁺ |
| B.512 | 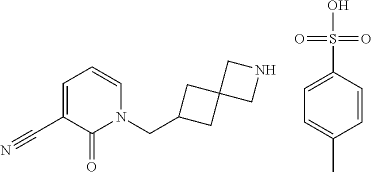 [SMILES: N#Cc1cccn(CC2CC3(CNC3)C2)c1=O ; Cc1ccc(S(=O)(=O)O)cc1] | 1-(2- azaspiro[3.3]heptan-6- ylmethyl)-2-oxo- pyridine-3- carbonitrile; 4- methylbenzenesulfonic acid | 2- hydroxypyridine- 3-carbonitrile (CAS: 609-71-2) | 230.2 [M − TsOH + H]⁺ |
| B.513 | 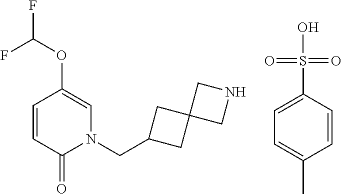 [SMILES: O=c1ccc(OC(F)F)cn1CC1CC2(CNC2)C1 ; Cc1ccc(S(=O)(=O)O)cc1] | 1-(2- azaspiro[3.3]heptan-6- ylmethyl)-5- (difluoromethoxy) pyridin-2-one; 4- methylbenzenesulfonic acid | 5- (difluoromethoxy)- 1H-pyridin-2- one (CAS: 130318-75-1) | 271.2 [M − TsOH + H]⁺ |
| B.538 | 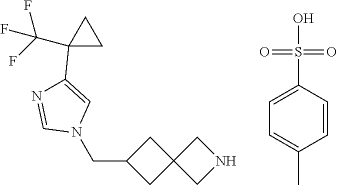 [SMILES: FC(F)(F)C1(c2cn(CC3CC4(CNC4)C3)cn2)CC1 ; Cc1ccc(S(=O)(=O)O)cc1] | 6-[[4-[1- (trifluoromethyl) cyclopropyl] imidazol-1- yl]methyl]-2- azaspiro[3.3]heptane; 4- methylbenzenesulfonic acid | 4-[1- (trifluoromethyl) cyclopropyl]-1H- imidazole (C.18) | 286.2 [M − TsOH + H]⁺ |
| | (regioisomer B.538 | | | |
| | generated concurrently) | | | |

### Example P.50

### 1-(2-Azaspiro[3.3]heptan-6-ylmethyl)-4-(trifluoromethyl)pyridin-2-one; 4-methylbenzenesulfonic Acid

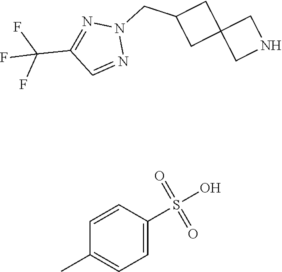 [SMILES: FC(F)(F)c1cnn(CC2CC3(CNC3)C2)n1 ; Cc1ccc(S(=O)(=O)O)cc1]

To a solution of tert-butyl 6-[[4-(trifluoromethyl)triazol-2-yl]methyl]-2-azaspiro[3.3]heptane-2-carboxylate (2.5 g, 7.2 mmol) in EtOAc (100 mL) was added p-toluenesulfonic acid monohydrate (4.12 g, 21.6 mmol). The reaction mixture was stirred at 25° C. for 18 h, filtered and washed with diethyl ether to afford the title compound (3.63 g, 81% yield) as white powder. MS (ESI): m/z=247.2 [M+H]⁺.

### Step a) Tert-Butyl 6-[[4-(trifluoromethyl)triazol-2-yl]methyl]-2-azaspiro[3.3]heptane-2-carboxylate

To a mixture of 4-(trifluoromethyl)-1H-triazole (2.69 g, 19.7 mmol), lithium bromide (3.41 g, 39.3 mmol) in acetonitrile (300 mL) was added tert-butyl 6-(methylsulfonyloxymethyl)-2-azaspiro[3.3]heptane-2-carboxylate (P.23, Step a) (6.0 g, 19.65 mmol, 1 eq, CAS 2740574-92-7). The reaction mixture was stirred at 50° C. for 18 h, filtered and concentrated in vacuo. The crude material was purified by flash chromatography (silica gel, 220 g, 0% to 100% MTBE in heptane) to afford the title compound (2.5 g, 36% yield) as light yellow solid. MS (ESI): m/z=247.0 [M+H-Boc]⁺.

In analogy to Example P.50, the following building block was generated using the relevant commercial building block in Step a). In some cases sodium iodide was used in place of lithium bromide in Step a).

| | | | Building | MS, ESI: |
|---|---|---|---|---|
| Ex. | Structure | Systematic Name | Blocks | m/z |
| B.354 | 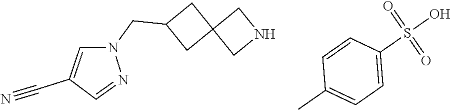 | 1-(2- azaspiro[3.3]heptan-6- ylmethyl)pyrazole-4- carbonitrile; 4- methylbenzenesulfonic acid | 1H-pyrazole-4- carbonitrile (CAS: 31108-57-3) | 203.0 [M − TsOH + H]⁺ |
| B.399 | 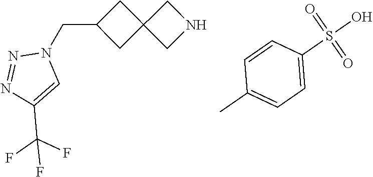 [SMILES: FC(F)(F)c1cn(CC2CC3(CNC3)C2)nn1 ; Cc1ccc(S(=O)(=O)O)cc1] | 6-[[4- (trifluoromethyl)triazo 1-1-yl]methyl]-2- azaspiro[3.3]heptane; 4- methylbenzenesulfonic acid | 4- (trifluoromethyl)- 1H-triazole | 247.0 [M − TsOH + H]⁺ |
| | (generated as minor | | | |
| | regioisomer along with | | | |
| | P.50 in Step a) | | | |
| B.418 | 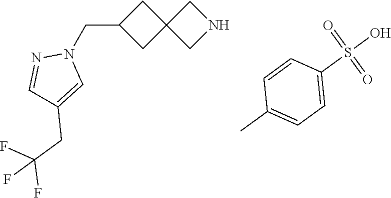 [SMILES: FC(F)(F)Cc1cnn(CC2CC3(CNC3)C2)c1 ; Cc1ccc(S(=O)(=O)O)cc1] | 6-[[4-(2,2,2- trifluoroethyl)pyrazol- 1-yl]methyl]-2- azaspiro[3.3]heptane; 4- methylbenzenesulfonic acid | 4-(2,2,2- trifluoroethyl)- 1H-pyrazole (CAS: 1211529- 61-1) | 260.2 [M − TsOH + H]⁺ |

### Example P.62

### 6-[[3-(Difluoromethyl)-1H-pyrazol-5-yl]methyl]-2-azaspiro[3.3]heptane; 2,2,2-trifluoroacetic Acid

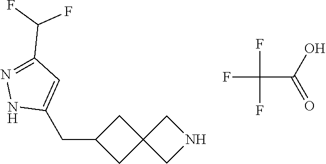 [SMILES: FC(F)c1cc(CC2CC3(CNC3)C2)[nH]n1 ; O=C(O)C(F)(F)F]

To a solution of tert-butyl 6-[[5-(difluoromethyl)-2-(2-trimethylsilylethoxymethyl)pyrazol-3-yl]methyl]-2-azaspiro[3.3]heptane-2-carboxylate (6 g, 13.1 mmol) in dichloromethane (40 mL) was added 2,2,2-trifluoroacetic acid (20 mL) at 0° C. The mixture was stirred at 20° C. for 16 h. The reaction mixture was concentrated in vacuo. The residue was purified by prep-HPLC to afford the title compound (3.94 g, 87% yield) as white solid. MS (ESI): m/z=228.2 [M+H]⁺.

### Step a) Tert-Butyl 6-[[5-(difluoromethyl)-2-(2-trimethylsilylethoxymethyl)pyrazol-3-yl]methylene]-2-azaspiro[3.3]heptane-2-carboxylate

To a mixture of 2-[[5-bromo-3-(difluoromethyl)pyrazol-1-yl]methoxy]ethyl-trimethyl-silane (5.3 g, 16.2 mmol, CAS 2416163-95-4), tert-butyl 6-[(4,4,5,5-tetramethyl-1,3,2-dioxaborolan-2-yl)methylene]-2-azaspiro[3.3]heptane-2-carboxylate (5.97 g, 17.8 mmol, CAS 2763647-64-7) and potassium carbonate (4.47 g, 32.4 mmol) in 1,4-dioxane (50 mL) and water (5 mL) was added cyclopenta-2,4-dien-1-yl(diphenyl)phosphane; dichloromethane; dichloropalladium; iron(2+) (1.32 g, 1.62 mmol, 0.1 eq, CAS 95464-05-4) under N₂. The mixture was stirred at 100° C. for 2 h under N₂ atmosphere. The reaction mixture was poured into water (500 mL). The aqueous phase was extracted with EtOAc (300 mL×2). The organic phase was washed with brine (600 mL), dried with anhydrous Na₂SO₄, filtered and concentrated in vacuo. The residue was purified by column chromatography (SiO₂, petroleum ether/ethyl acetate=100/1 to 1/1) to afford the title compound (6.1 g, 13.4 mmol, 79% yield) as alight yellow solid. MS (ESI): m/z=456.3 [M+H]⁺.

### Step b) Tert-Butyl 6-[[5-(difluoromethyl)-2-(2-trimethylsilylethoxymethyl)pyrazol-3-yl]methyl]-2-azaspiro[3.3]heptane-2-carboxylate

To a solution of tert-butyl 6-[[5-(difluoromethyl)-2-(2-trimethylsilylethoxymethyl)pyrazol-3-yl]methylene]-2-azaspiro[3.3]heptane-2-carboxylate (7 g, 15.4 mmol) in EtOAc (100 mL) was added Pd/C 10% (2 g, 4.61 mmol, 0.3 eq) under N₂ atmosphere. The mixture was stirred at 25° C. for 0.5 h under H₂ (15 PSI) atmosphere. The reaction mixture was filtered and concentrated in vacuo to afford tert-butyl 6-[[5-(difluoromethyl)-2-(2-trimethylsilylethoxymethyl)pyrazol-3-yl]methyl]-2-azaspiro[3.3]heptane-2-carboxylate (6 g, 85% yield) as a colorless oil.

### Example B.329

### [4-(2-azaspiro[3.3]heptan-6-ylmethyl)phenyl]imino-oxo-(trifluoromethyl)-λ⁶-sulfane; 4-methylbenzenesulfonic Acid

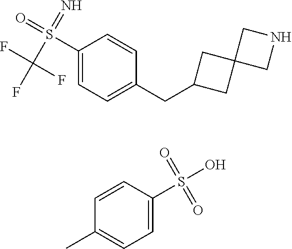 [SMILES: N=S(=O)(c1ccc(CC2CC3(CNC3)C2)cc1)C(F)(F)F ; Cc1ccc(S(=O)(=O)O)cc1]

To a mixture of tert-butyl 6-[[2-fluoro-4-(trifluoromethylsulfonimidoyl)phenyl]methyl]-2-azaspiro[3.3]heptane-2-carboxylate (2.03 g, 4.65 mmol) in EtOAc (20 mL) was added p-toluenesulfonic acid (0.96 g, 5.58 mmol) at 20° C. Then the mixture was stirred at 80° C. for 12 h. The mixture was concentrated to remove the solvent, then added deionized water and lyophilized to give the title compound (2.05 g, 4.03 mmol, 83.2% yield) as yellow gum. MS (ESI): m/z=337.1 [M-TsOH+H]⁺

### Step a) Tert-Butyl 6-[[Z fluoro-4-(trifluoromethylsulfanyl)phenyl]methylene]-2-azaspiro[3.3]heptane-2-carboxylate

To a solution of tert-butyl 6-[(4,4,5,5-tetramethyl-1,3,2-dioxaborolan-2-yl)methylene]-2-azaspiro[3.3]heptane-2-carboxylate (15.6 g, 46.5 mmol), 1-bromo-2-fluoro-4-(trifluoromethylsulfanyl)benzene (CAS: 1520947-39-0) (12.8 g, 46.5 mmol) and POTASSIUM CARBONATE (12.9 g, 93.1 mmol) in 1,4-Dioxane (170 mL) and water (34 mL) was added 1,1′-bis(diphenylphosphino)ferrocene-palladium(II)dichloride dichloromethane complex (3.8 g, 4.65 mmol) at 25° C., then the mixture was stirred at 80° C. under N₂ atmosphere for 12 h. The mixture was purified by chromatography on silica gel (PE:EA=5:1) and concentrated under vacuum to give a crude product, which was further purified by PREP-HPLC to give the title compound (5.3 g, 13.1 mmol, 28% yield) as a yellow oil. MS (ESI): m/z=348.0 [M-C₄H₈+H]⁺

### Step b) of Tert-Butyl 6-[[2 fluoro-4-(trifluoromethylsulfanyl)phenyl]methyl]-2-azaspiro[3.3]heptane-2-carboxylate

To a solution of tert-butyl 6-[[2-fluoro-4-(trifluoromethylsulfanyl)phenyl]methylene]-2-azaspiro[3.3]heptane-2-carboxylate (5.3 g, 13.1 mmol) in EtOAc (80 mL) was added wet Pd/C (1.8 g, 1.31 mmol) at 25° C. under N₂, the mixture was stirred at 25° C. under H₂ atmosphere (balloon) for 12 h. The mixture was then filtered and the filtrate was concentrated to give the title compound (5.5 g, 13.6 mmol, 95.0% yield) as colorless oil. MS (ESI): m/z=350.0 [M-C₄H₈+H]⁺

### Step c) Tert-Butyl 6-[[2-fluoro-4-(trifluoromethylsulfonimidoyl)phenyl]methyl]-2-azaspiro[3.3]heptane-2-carboxylate

To a soution of tert-butyl 6-[[2-fluoro-4-(trifluoromethylsulfanyl)phenyl]methyl]-2-azaspiro[3.3]heptane-2-carboxylate (5.3 g, 13.1 mmol) in trifluoroethanol (55.0 mL) was added PhI(OAc)₂ (17.7 g, 54.9 mmol) and NH₂COONH₄ (3.06 g, 39.2 mmol) at 25° C. Then the mixture was stirred at 60° C. for 12 h. The mixture was purified by PREP-HPLC (Phenomenex luna C18 150*40 mm*1 5 um water(FA)-ACN) to give the title compound (2.03 g, 4.65 mmol, 36% yield) as yellow oil. MS (ESI): m/z=381.1 [M-C₄H₈+H]⁺

In analogy to Example B.329, the following building block was generated using the relevant commercial building block in Step a).

| | | | Building | MS, ESI: |
|---|---|---|---|---|
| Ex. | Structure | Systematic Name | Blocks | m/z |
| B.497 | 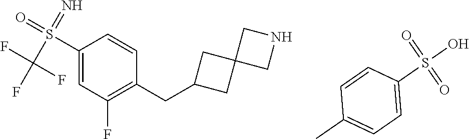 [SMILES: N=S(=O)(c1ccc(CC2CC3(CNC3)C2)c(F)c1)C(F)(F)F ; Cc1ccc(S(=O)(=O)O)cc1] | [4-(2- azaspiro[3.3]heptan-6- ylmethyl)-3-fluoro- phenyl]-imino-oxo- (trifluoromethyl)-2.6- sulfane; 4- methylbenzenesulfonic acid | 1-bromo-2- fluoro-4- (trifluoromethyl- sulfanyl) benzene (CAS: 1520947- 39-0) | 337.1 [M − TsOH + H]⁺ |

### Example B.334

### 5-(2-azaspiro[3.3]heptan-6-yloxy)-2-(trifluoromethyl)benzonitrile; 4-methylbenzenesulfonic Acid

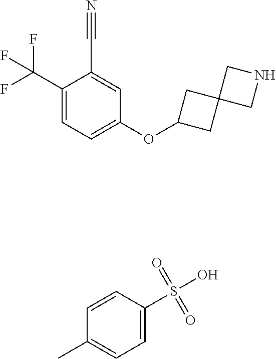 [SMILES: N#Cc1cc(OC2CC3(CNC3)C2)ccc1C(F)(F)F ; Cc1ccc(S(=O)(=O)O)cc1]

To a solution of tert-butyl 6-[3-cyano-4-(trifluoromethyl)phenoxy]-2-azaspiro[3.3]heptane-2-carboxylate (452 mg, 1.18 mmol) in Ethyl acetate (10 mL) p-toluenesulfonic acid monohydrate (292 mg, 1.54 mmol) was added. The mixture was stirred at 20° C. for 16 h, the precipitated solid was filtered, washed with ACN and dried to give the title compound (283 mg, 0.62 mmol, 53% yield) as a white solid. MS (ESI): m/z=283.2 [M-TsOH+H]⁺

### Step a) Tert-Butyl 6-[3-cyano-4-(trifluoromethyl)phenoxy]-2-azaspiro[3.3]heptane-2-carboxylate

A solution of tert-butyl 6-hydroxy-2-azaspiro[3.3]heptane-2-carboxylate (500 mg, 2.34 mmol) and 5-hydroxy-2-(trifluoromethyl)benzonitrile (483 mg, 2.58 mmol) in toluene (10 mL) was cooled to 0° C. Triphenylphosphine (799 mg, 3.05 mmol) and diisopropyl azodicarboxylate (0.6 mL, 3.05 mmol) were added under Ar. The mixture was warmed to 20° C. and stirred for 16 h. The mixture was concentrated, the residue was triturated with TBME. The precipitated solid was filtered off, the filtrate was concentrated. The residue was purified by FC (silica, 20% EtOAc in hexane) to afford the title compound (430 mg, 1.12 mmol, 48% yield) as a white solid. LCMS: molecular peak is not shown.

In analogy to Example B.334, the following building block was generated using the relevant commercial building block in Step a).

| | | | Building | MS, ESI: |
|---|---|---|---|---|
| Ex. | Structure | Systematic Name | Blocks | m/z |
| B.376 | 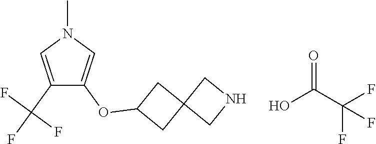 [SMILES: Cn1cc(OC2CC3(CNC3)C2)c(C(F)(F)F)c1 ; O=C(O)C(F)(F)F] | 6-[1-methyl-3- (trifluoromethyl) pyrazol-4-yl]oxy-2- azaspiro[3.3]heptane; 2,2,2-trifluoroacetic acid | 1-methyl-3- (trifluoromethyl) pyrazol-4-ol (CAS: 172834-79-6) | 262.2 [M − TFA + H]⁺ |
| B.463 | 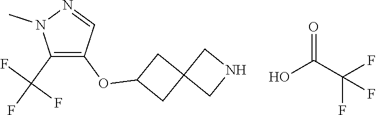 [SMILES: Cn1ncc(OC2CC3(CNC3)C2)c1C(F)(F)F ; O=C(O)C(F)(F)F] | 6-[1-methyl-5- (trifluoromethyl) pyrazol-4-yl]oxy-2- azaspiro[3.3]heptane; 2,2,2-trifluoroacetic acid | 1-methyl-5- (trifluoromethyl) pyrazol-4-ol (CAS: 161038-53-5) | 262.0 [M − TFA + H]⁺ |
| B.464 | 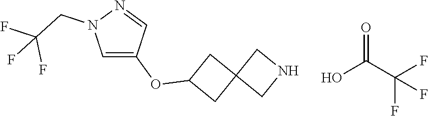 [SMILES: FC(F)(F)Cn1cc(OC2CC3(CNC3)C2)cn1 ; O=C(O)C(F)(F)F] | 6-[1-(2,2,2- trifluoroethyl)pyrazol- 4-yl]oxy-2- azaspiro[3.3]heptane; 2,2,2-trifluoroacetic acid | 1-(2,2,2- trifluoroethyl) pyrazol- 4-ol (CAS: 1598936-56-1) | 262.0 [M − TFA + H]⁺ |

### Example B.336

### 6-[3-(trifluoromethoxy)phenyl]sulfonyl-2-azaspiro[3.3]heptane; 2,2,2-trifluoroacetic Acid

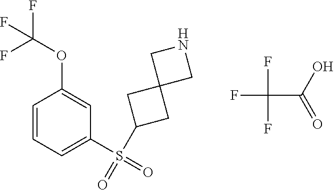

To a solution of 7-[3-(trifluoromethyl)phenyl]sulfonyl-2-azaspiro[3.5]nonane-2-carboxylic acid tert-butyl ester (267 mg, 0.585 mmol) in dichloromethane (3 mL) was added TFA (667 mg, 451 μL, 5.85 mmol) and the reaction mixture was stirred at RT for 18 h. Volatiles were removed in vacuo to give 431 mg of the crude title compound as colorless viscous oil (purity˜60% major contaminant excess of TFA), which was used without further purification. MS (ESI): m/z=322.1 [M-TsOH+H]⁺

### Step a) Tert-Butyl 6-[[2-fluoro-4-(trifluoromethylsulfonimidoyl)phenyl]methyl]-2-azaspiro[3.3]heptane-2-carboxylate

To a solution of 6-methylsulfonyloxy-2-azaspiro[3.3]heptane-2-carboxylic acid tert-butyl ester (1000 mg, 3.26 mmol) in tetrahydrofuran (15 mL) was added 3-(trifluoromethoxy)benzenethiol (696 mg, 3.59 mmol) and cesium carbonate (1.06 g, 3.26 mmol) after which the reaction mixture was stirred at 60° C. for 18 h. A further addition of of 3-(trifluoromethoxy)benzenethiol (348 mg) and cesium carbonate (503 mg) was made, after which the reaction mixture was stirred again at 60° C. for 6 h. The reaction mixture was poured into a separating funnel containing ethyl acetate and aq. Na₂CO₃ 1 M solution for extraction. The organic phase was collected and the aqueous phase was back-extracted with ethyl acetate. The combined organic phases were dried over sodium sulfate and evaporated down to dryness. The crude material was purified by flash chromatography (silica gel, ethyl acetate in heptane (5% to 25%) to yield 1.11 g of the title compound. MS (ESI): m/z=334.0 [M-C₄H₈+H]⁺

### Step b) 7-[3-(trifluoromethyl)phenyl]sulfonyl-2-azaspiro[3.5]nonane-2-carboxylic Acid Tert-Butyl Ester

To a suspension of 6-[[3-(trifluoromethoxy)phenyl]thio]-2-azaspiro[3.3]heptane-2-carboxylic acid tert-butyl ester (250 mg, 0.629 mmol) in a mixture of methanol (3 mL)/water (3 mL) was added oxone (812 mg, 1.32 mmol) and sodium bicarbonate (159 mg, 1.89 mmol) after which the reaction mixture was stirred at RT for 18 h. The reaction mixture was poured into a separating funnel containing ethyl acetate and aq. sol. 1 M NaHCO₃, after extraction the organic phase was collected and the aqueous phase was back-extracted with ethyl acetate. The combined organic phases were dried over sodium sulfate and evaporated down to dryness to 265 mg of the crude title compound as a white solid. MS (ESI): m/z=366.1 [M-C₄H₈+H]⁺

In analogy to Example B.336, the following building block was generated using the relevant commercial building blocks.

| | | | Building | MS, ESI: |
|---|---|---|---|---|
| Ex. | Structure | Systematic Name | Blocks | m/z |
| B.337 | 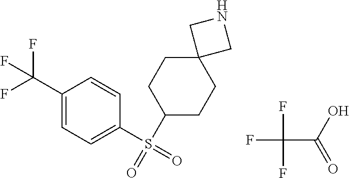 [SMILES: O=S(=O)(c1ccc(C(F)(F)F)cc1)C1CCC2(CC1)CNC2 ; O=C(O)C(F)(F)F] | 7-[4- (trifluoromethyl) phenyl]sulfonyl-2- azaspiro[3.5]nonane; 2,2,2-trifluoroacetic acid | 4- (trifluoromethyl) benzenethiol; 7- methylsulfonyloxy- 2- azaspiro[3.5] nonane-2- carboxylic acid tert-butyl ester (CAS: 1421270-78-1) | 334.1 [M − TsOH + H]⁺ |
| B.338 | 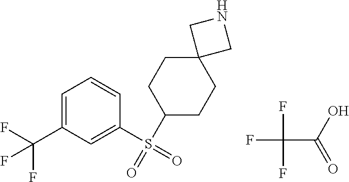 [SMILES: O=S(=O)(c1cccc(C(F)(F)F)c1)C1CCC2(CC1)CNC2 ; O=C(O)C(F)(F)F] | 7-[3- (trifluoromethyl) phenyl]sulfonyl-2- azaspiro[3.5]nonane; 2,2,2-trifluoroacetic acid | 3- (trifluoromethyl) benzenethiol; 7- methylsulfonyloxy- 2- azaspiro[3.5] nonane-2-carboxylic acid tert-butyl ester (CAS: 1421270-78-1) | 334.1 [M − TsOH + H]⁺ |

### Example B.339

### 2-azaspiro[3.3]heptan-6-yl-imino-oxo-[3-(trifluoromethoxy)phenyl]-λ⁶-sulfane; 2,2,2-trifluoroacetic Acid

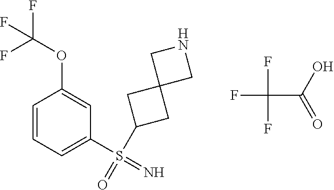

To a solution of 6-[[3-(trifluoromethoxy)phenyl]sulfonimidoyl]-2-azaspiro[3.3]heptane-2-carboxylic acid tert-butyl ester (225 mg, 0.508 mmol) in dichloromethane (2 mL) was added TFA (580 mg, 392 μL, 5.08 mmol) and the reaction mixture was stirred at RT for 18 h. Volatiles were removed in vacuo to give 440 mg of the crude title compound (purity ˜50%, major contaminant excess of TFA) as a yellow viscous oil, which was used without further purification. MS (ESI): m/z=321.1 [M-TsOH+H]⁺

### Step a) 6-[[3-(trifluoromethoxy)phenyl]sulfonimidoyl]-2-azaspiro[3.3]heptane-2-carboxylic Acid Tert-Butyl Ester

To a solution of 6-[[3-(trifluoromethoxy)phenyl]thio]-2-azaspiro[3.3]heptane-2-carboxylic acid tert-butyl ester (B.336, Step a) (300 mg, 0.755 mmol) in methanol (1.5 mL) was added iodobenzene diacetate (608 mg, 1.89 mmol) and ammonium carbamate (58.9 mg, 0.755 mmol) after which the reaction mixture was stirred at RT for 3 h. The crude reaction solution was absorbed onto H-MN isolute and dried under vacuum followed by direct purification by flash chromatography with a SiO₂ column (eluent mixture of heptane and a solution (EtOAc:EtOH 3:1) (5% to 50%)) to give 228 mg of the title compound as a colorless gum. MS (ESI): m/z=421.1 [M+H]⁺

### Example B.349

### 3-(2-azaspiro[3.3]heptan-6-ylmethyl)-5-(trifluoromethyl)-1,2,4-thiadiazole; 4-methylbenzenesulfonic Acid

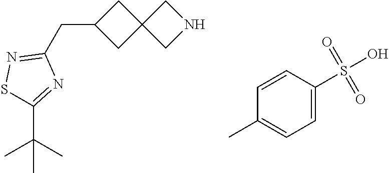 [SMILES: CC(C)(C)c1nc(CC2CC3(CNC3)C2)ns1 ; Cc1ccc(S(=O)(=O)O)cc1]

A mixture of tert-butyl 6-[[5-(trifluoromethyl)-1,2,4-thiadiazol-3-yl]methyl]-2-azaspiro[3.3]heptane-2-carboxylate (400 mg, 1.1 mmol) and TsOH (227 mg, 1.32 mmol) in EtOAc (5 mL) was stirred at 80° C. for 3 h. The reaction mixture was concentrated under reduced pressure and lyophilized to afford the title compound (415 mg, 0.95 mmol, 85.3% yield) as a white solid. MS (ESI): m/z=264.0 [M-TsOH+H]⁺

### Step a) Tert-Butyl 6-(cyanomethyl)-2-azaspiro[3.3]heptane-2-carboxylate

To a solution of tert-butyl 6-(methylsulfonyloxymethyl)-2-azaspiro[3.3]heptane-2-carboxylate (2740574-92-7) (5.0 g, 16.4 mmol) in DMSO (70 mL) was added potassium cyanide (2.45 g, 37.7 mmol) at room temperature. The resulting solution was stirred for 18 h at 80° C. The reaction was quenched by water (50 mL) and then extracted with TBME (3×100 mL). The organic layers were washed with brine, dried over Na₂SO₄, filtered and concentrated in vacuum to give the title compound (3.4 g, 14.4 mmol, 83.6% yield) as white solid. MS (ESI): m/z=181.0 [M-C₄H₈+H]⁺.

### Step b) Tert-Butyl 6-[2-(hydroxyamino)-2-imino-ethyl]-2-azaspiro[3.3]heptane-2-carboxylate

To a mixture of hydroxylamine; hydrochloride (2353 mg, 33.9 mmol) in ethanol (25 mL) was added TEA (3426 mg, 33.9 mmol) at 25° C. After 1 h, tert-butyl 6-(cyanomethyl)-2-azaspiro[3.3]heptane-2-carboxylate (4000 mg. 16.9 mmol) added to the reaction mixture above, then the reaction was stirred at 50° C. for further 12 h. The reaction was concentrated under reduced pressure to give a residue. The residue was dissolved in water (50.0 mL), extracted with ethyl acetate (50.0 mL×3), the combined extracts were concentrated under reduced pressure to give the title compound (4.5 g, 16.7 mmol, 98.7% yield) as a colorless oil, which was used for next step without further purification. MS (ESI): m/z=270.1 [M+H]⁺

### Step c) Tert-Butyl 6-(2-amino-2-imino-ethyl)-2-azaspiro[3.3]heptane-2-carboxylate; Acetic Acid

To a solution of tert-butyl 6-[2-(hydroxyamino)-2-imino-ethyl]-2-azaspiro[3.3]heptane-2-carboxylate (4.5 g, 16.7 mmol) and Ac₂O (2.56 g, 25.1 mmo) in Acetic acid (20 mL) was added Pd/C(wet) (1.2 g) at 25° C. Then the reaction was stirred at 25° C. under H₂ atmosphere(H₂ balloon) for 12 h. The reaction mixture was filtered, the filtrate was concentrated under reduced pressure to give the crude title compound (5.2 g, 16.6 mmol, 99.3% yield) as a light yellow oil. MS (ESI): m/z=254.0 [M-AcOH+H]⁺

### Step d) Tert-Butyl 6-[(5-amino-1,2,4-thiadiazol-3-yl)methyl]-2-azaspiro[3.3]heptane-2-carboxylate

To a solution of tert-butyl 6-(2-amino-2-imino-ethyl)-2-azaspiro[3.3]heptane-2-carboxylate; acetic acid (3000 mg, 9.57 mmol) in water (30 mL) was added sodium hypochlorite (15.0 mL, 10.5 mmol) dropwise at 0° C., then the mixture was stinted at 20° C. for 1 h, then diluted with water and extracted with EtOAc (20 mL×3), the combined organic phase was dried over Na₂SO₄ and concentrated, the residue was dissolved in Methanol (30 mL), potassium thiocyanate (1023 mg, 10.5 mmol) was added at 0° C., then the solution was stirred at 20° C. for 11 h. The reaction mixture was filtered, the filtrate was concentrated under reduced pressure to give a residue. The residue was purified by column on silica (ethyl acetate:petroleum ether 0-80%) and concentrated under reduced pressure to give the title compound (1400 mg, 4.51 mmol, 47.1% yield) as a brown solid. MS (ESI): m/z=311.0 [M+H]⁺

### Step e) Tert-Butyl 6-[(5-iodo-1,2,4-thiadiazol-3-yl)methyl]-2-azaspiro[3.3]heptane-2-carboxylate

To a mixture of tert-butyl 6-[(5-amino-1,2,4-thiadiazol-3-yl)methyl]-2-azaspiro[3.3]heptane-2-carboxylate (1400 mg, 4.51 mmol and CuI (1718 mg, 9.02 mmol) in MeCN (50 mL) was added a solution of tert-butyl nitrite (930 mg, 9.02 mmol, 2.0 eq) in MeCN (5 mL) at 25° C., then the reaction was stirred at 70° C. under N₂ atmosphere for 12 h. The reaction mixture was filtered, the filtrate was concentrated under reduced pressure to give a residue. The residue was purified by column on silica (ethyl acetate:petroleum ether 0-40%) and concentrated under reduced pressure to give the title compound (900 mg, 2.14 mmol, 47.4% yield) as a yellow solid. MS (ESI): m/z=421.9 [M+H]⁺

### Step f) Tert-Butyl 6-[[S-(trifluoromethyl)-1,2,4-thiadiazol-3 yl]methyl]-2-azaspiro[3.3]heptane-2-carboxylate

A mixture of tert-butyl 6-[(5-iodo-1,2,4-thiadiazol-3-yl)methyl]-2-azaspiro[3.3]heptane-2-carboxylate (890 mg. 2.11 mmol), Diphenyl(trifluoromethyl)sulfonium trifluoromethanesulfonate (1025 mg, 2.54 mmol) and CuI (1207 mg, 6.34 mmol) in DMF (8 mL) was stirred at 60° C. under N₂ atmosphere for 12 h. The reaction mixture was filtered, the filtrate was concentrated under reduced pressure to give a residue. The residue was purified by reverse flash (FA condition; MeCN:H₂O=0-70%) and lyophilized to afford a residue. This residue was purified by column on silica (ethyl acetate:petroleum ether 0-30%) and concentrated under reduced pressure to give the title compound (400 mg, 1.1 mmol, 52.1% yield) as a colorless oil. MS (ESI): m/z=307.9 [M-C₄H₈+H]⁺

### Example B.358

### 4-(2-azaspiro[3.3]heptan-6-ylmethyl)-2-(trifluoromethoxy)benzamide; 4-methylbenzenesulfonic Acid

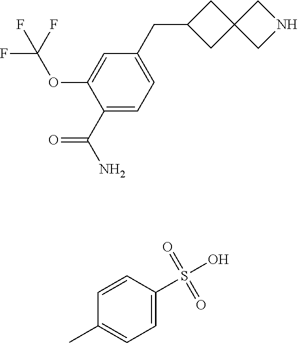 [SMILES: NC(=O)c1ccc(CC2CC3(CNC3)C2)cc1OC(F)(F)F ; Cc1ccc(S(=O)(=O)O)cc1]

To a solution of 6-[4-carbamoyl-3-(trifluoromethoxy)benzyl]-2-azaspiro[3.3]heptane-2-carboxylic acid tert-butyl ester (63 mg, 0.152 mmol) in ethyl acetate (1.01 mL) was added p-toluenesulfonic acid monohydrate (30.4 mg, 0.160 mmol). The reaction mixture was stirred overnight at reflux. Solvents were removed and product used without further purification for next step. MS (ESI): m/z=315.1 [M+H]⁺

### Step a) 6-[4-carbomethoxy-3-(trifluoromethoxy)benzylidene]-2-azaspiro[3.3]heptane-2-carboxylic Acid Tert-Butyl Ester

4-bromo-2-(trifluoromethoxy)benzoic acid methyl ester (981 mg, 603 μL, 3.28 mmol) and 6-[(4,4,5,5-tetramethyl-1,3,2-dioxaborolan-2-yl)methylene]-2-azaspiro[3.3]heptane-2-carboxylic acid tert-butyl ester (1000 mg, 2.98 mmol) were dissolved in a degassed solution of 1,4-dioxane (149 mL) and water (14.9 mL). The reaction mixture was degassed for 5 min again before addition of 1,1′-bis(di-tert-butylphosphino)ferrocene-palladium dichloride (97.2 mg, 0.149 mmol) followed by tripotassium phosphate (1.27 g, 5.97 mmol). The reaction mixture was then stirred at room temp. for 3.5 h. The reaction mixture was poured into EtOAc, washed with water. The aqueous layer was extracted back twice. Combined organic layers were washed with brine, dried over Na₂SO₄, filtered and concentrated under reduced pressure. The resulting crude was then purified by flash chromatography eluting with a gradient Heptane/EtOAc 0-35% to afford the title compound as a white solid. MS (ESI): m/z=372.1 [M-C₄H₈+H]⁺

### Step b) 6-[4-carbomethoxy-3-(trifluoromethoxy)benzyl]-2-azaspiro[3.3]heptane-2-carboxylic Acid Tert-Butyl Ester

6-[4-carbomethoxy-3-(trifluoromethoxy)benzyl idene]-2-azaspiro[3.3]heptane-2-carboxylic acid tert-butyl ester (1050 mg, 2.46 mmol) was dissolved in methanol (81.9 mL) and tetrahydrofuran (40.9 mL). The reaction mixture was degassed for 10 min with argon. Then, still under argon, platinum (IV) oxide (112 mg, 0.491 mmol) was added to the mixture. The argon atmosphere was replaced by hydrogen (via balloon), and the reaction mixture stirred under hydrogen atmosphere for 1 h. The reaction mixture was filtrated, and the resulting solution was concentrated under reduced pressure to afford the crude title compound as an oil, which was used directly in the next step without further purification. MS (ESI): m/z=374.1 [M-C₄H₈+H]⁺

### Step c) 4-[(2-tert-butoxycarbonyl-2-azaspiro[3.3]heptan-6 yl)methyl]-2-(trifluoromethoxy) Benzoic Acid

To a solution of 6-[4-carbomethoxy-3-(trifluoromethoxy)benzyl]-2-azaspiro[3.3]heptane-2-carboxylic acid tert-butyl ester (460 mg, 1.04 mmol) in tetrahydrofuran (2.6 mL) and methanol (2.6 mL) was added 1 M NaOH aqueous solution (4.16 mL, 4.16 mmol) at room temperature. The mixture was then heated at 70° C. for 48 h. Organic solvent was removed under reduced pressure and then the resulting crude material was acidified until pH 2. Then the aqueous layer was extracted three times with EtOAc. Organic layers were combined, dried over Na₂SO₄, filtered off and concentrated under reduced pressure to yield a crude product that was used directly in the next step without further purification. MS (ESI): m/z=360.1 [M-C₄H₈+H]⁺

### Step d) 6-[4-carbamoyl-3-(trifluoromethoxy)benzyl]-2-azaspiro[3.3]heptane-2-carboxylic Acid Tert-Butyl Ester

A solution of 4-[(2-tert-butoxycarbonyl-2-azaspiro[3.3]heptan-6-yl)methyl]-2-(trifluoromethoxy)benzoic acid (440 mg, 1.06 mmol) in dichloromethane (5.3 mL) was cooled at 0° C. CDI (177 mg, 1.06 mmol) was added and the resulting mixture was stirred 15 min. 2 M NH₃ in isopropanol solution (2.65 mL, 5.3 mmol) was then added and the reaction mixture was stirred overnight at room temp. A further addition of 2 M NH₃ in isopropanol solution (2.65 mL, 5.3 mmol) was made and the reaction stirred for 48 h. The reaction mixture was washed with 1 M HCl (aq) twice, and with water. The aqueous layer was back-extracted with dichloromethane. The combined organic layers were dried over Na₂SO₄ and concentrated under reduced pressure. The crude material was then purified by flash chromatography using hexane/EtOAc to afford the title compound as a viscous oil. MS (ESI): m/z=359.1 [M-C₄H₈+H]⁺

In analogy to Example B.358, the following building block was generated using the relevant commercial building blocks.

| | | | Building | MS, ESI: |
|---|---|---|---|---|
| Ex. | Structure | Systematic Name | Blocks | m/z |
| B.359 | 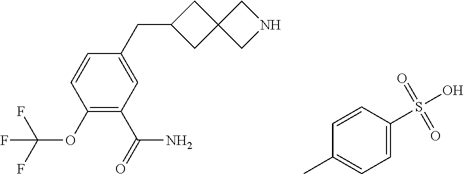 [SMILES: NC(=O)c1cc(CC2CC3(CNC3)C2)ccc1OC(F)(F)F ; Cc1ccc(S(=O)(=O)O)cc1] | 5-(2- azaspiro[3.3]heptan-6- ylmethyl)-2- (trifluoromethoxy) benzamide; 4- methylbenzenesulfonic acid | 5-bromo-2- (trifluoromethoxy) benzoic acid methyl ester | 315.1 [M − TsOH + H]⁺ |

### Example B.366

### 2-[5-(trifluoromethyl)-2-pyridyl]-2,6-diazaspiro[3.3]heptane; 4-methylbenzenesulfonic Acid

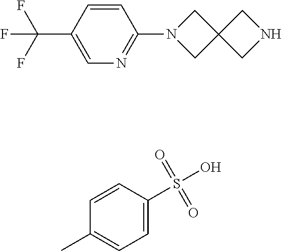 [SMILES: FC(F)(F)c1ccc(N2CC3(CNC3)C2)nc1 ; Cc1ccc(S(=O)(=O)O)cc1]

In a flask was added 6-[5-(trifluoromethyl)-2-pyridyl]-2,6-diazaspiro[3.3]heptane-2-carboxylic acid tert-butyl ester (285 mg, 0.830 mmol) and p-toluenesulfonic acid monohydrate (174 mg, 0.913 mmol) in ethyl acetate (7 mL), and the mixture was stirred at reflux overnight. Another equivalent of p-toluenesulfonic acid monohydrate (174 mg, 0.913 mmol) was added and the reaction stirred for a further 6 h. The solvent was evaporated and the crude was washed with diethyl ether to afford the crude title compound as an orange solid. MS (ESI): m/z=244.1 [M+H]⁺

### Step a) 6-[5-(trifluoromethyl)-2-pyridyl]2,6-diazaspiro[3.3]heptane-2-carboxylic Acid tert-butyl ester

In a flask was added 2-bromo-5-(trifluoromethyl)pyridine (250 mg, 1.11 mmol), 2-Boc-2,6-diazaspiro[3.3]heptane (241 mg, 1.22 mmol) and cesium carbonate (721 mg, 2.21 mmol) in 1,4-dioxane (5.38 mL) The suspension was bubbled with N₂ for 5 min and chloro(2-dicyclohexylphosphino-2′,6′-diisopropoxy-1,1′-biphenyl)[2-(2′-amino-1,1′-biphenyl)]palladium(II) (43.0 mg, 0.055 mmol) was added. The mixture was heated at 100° C. for 2 h. The mixture was diluted in EtOAc and filtered through celite, the filtrate was concentrated. Purification was performed by flash chromatography (heptane/EtOAc with gradient from 0 to 40% EtOAc to afford the title compound as a yellow solid. MS (ESI): m/z=344.1 [M+H]⁺

In analogy to Example B.367, the following building block was generated using the relevant commercial building blocks. In some cases different palladium/ligand catalysts were used in Step a) (e.g. Pd₂dba₃/Xantphos).

| | | | Building | MS, ESI: |
|---|---|---|---|---|
| Ex. | Structure | Systematic Name | Blocks | m/z |
| B.367 | 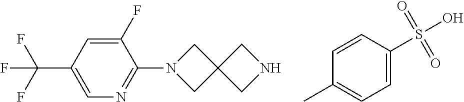 [SMILES: Fc1cc(C(F)(F)F)cnc1N1CC2(CNC2)C1 ; Cc1ccc(S(=O)(=O)O)cc1] | 2-[3-fluoro-5- (trifluoromethyl)-2- pyridyl]-2,6- diazaspiro[3.3]heptane: 4- methylbenzenesulfonic acid | 2-bromo-3- fluoro-5- (trifluoromethyl) pyridine (CAS: 89402-29-9) | 262.1 [M − TsOH + H]⁺ |
| B.378 | 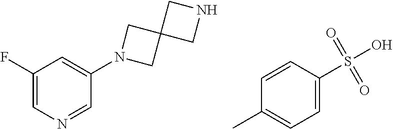 | 2-(5-fluoro-3- pyridyl)-2,6- diazaspiro[3.3] heptane;4- methylbenzenesulfonic acid | 3-bromo-5- fluoropyridine | 194.0 [M − TsOH + H]⁺ |

### Example B.377

### 6-[[4-[1-(trifluoromethyl)cyclopropyl]pyrazol-1-yl]methyl]-2-azaspiro[3.3]heptane; 4-methylbenzenesulfonic Acid

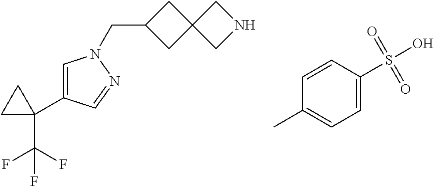 [SMILES: FC(F)(F)C1(c2cnn(CC3CC4(CNC4)C3)c2)CC1 ; Cc1ccc(S(=O)(=O)O)cc1]

A solution of tert-butyl 6-[[4-[1-(trifluoromethyl)cyclopropyl]pyrazol-1-yl]methyl]-2-azaspiro[3.3]heptane-2-carboxylate (400 mg, 1.04 mmol) and p-toluenesulfonic acid (214 mg, 1.25 mmol) in EtOAc (8 mL) was stuffed at 80° C. for 12 h. The reaction mixture was concentrated and lyophilized to give the title compound (467 mg, 1.02 mmol, 91% yield) as a light brown solid. MS (ESI): m/z=286.2 [M+H]⁺

### Step a) Tert-Butyl 6-[(4-bromopyrazol-1-yl)methylene]-2-azaspiro[3.3]heptane-2-carboxylate

To a solution of 4-bromopyrazole (2000 mg, 13.6 mmol) in DCE (40 mL) were added ten-butyl 6-[(4,4,5,5-tetramethyl-1,3,2-dioxaborolan-2-yl)methylene]-2-azaspiro[3.3]heptane-2-carboxylate (6840 mg, 20.4 mmol), pyridine (2153 mg, 27.2 mmol), boric acid (841 mg, 13.6 mmol) and Cu(OAc)₂ (3670 mg, 18.4 mmol). The mixture was stirred at 70° C. for 12 h under O₂. The reaction mixture was purified by prep-HPLC and lyophilized. The residue was triturated in petroleum ether (10 mL) and stirred for 10 min. The solid was collected by filtration to give the title compound (2867 mg, 8.09 mmol, 59% yield) as an off-white solid. MS (ESI): m/z=298.1 [M+H]⁺

### Step b) Tert-Butyl 6-[(4-bromopyrazol-1 yl)methyl]-2-azaspiro[3.3]heptane-2-carboxylate

To a solution of tert-butyl 6-[(4-bromopyrazol-1-yl)methylene]-2-azaspiro[3.3]heptane-2-carboxylate (2300 mg, 6.49 mmol) in EtOAc (25 mL) was added PtO₂ (920 mg, 4.05 mmol) at 20° C. under N₂, then the mixture was stirred at 20° C. under H₂ atmosphere (balloon) for 1 h. The precipitate was filtered off and the filtrate was dried in vacuo. The residue was purified over column chromatography (hexane/EtOAc, 1:1) to give the title compound (2200 mg, 6.18 mmol, 95% yield) as a light yellow solid.

MS (ESI): m/z=302.0 [M-C₄H₈+H]⁺

### Step c) Tert-Butyl 6-[[4-[1-(trifluoromethyl)vinyl]pyrazol-1-yl]methyl]-2-azaspiro[3.3]heptane-2-carboxylate

To a solution of tert-butyl 6-[(4-bromopyrazol-1-yl)methyl]-2-azaspiro[3.3]heptane-2-carboxylate (1700 mg, 4.77 mmol) in 1,4-Dioxane (20 mL) and water (4 mL) was added 1 (trifluoromethyl)vinylboronic acid hexylene glycol ester (1270 mg, 5.73 mmol), K₂CO₃ (1980 mg, 14.3 mmol) and [1,1′-BIS(DIPHENYLPHOSPHINO)FERROCENE]PALLADIUM (II) CHLORIDE (390 mg, 0.48 mmol) under N₂. The mixture was stirred at 80° C. for 12 h under N₂ atmosphere. The precipitate was filtered off and the filtrate was dried in vacuo. The residue was purified over column chromatography (PE/EA, 0-60%). The reaction mixture was purified by prep-HPLC, and lyophilized to give the title compound (1134.0 mg, 3.05 mmol, 64% yield) as light brown solid. MS (ESI): m/z=372.1 [M+H]⁺

### Step d) Tert-Butyl 6-[[4-[1-(trifluoromethyl)cyclopropyl]pyrazol-1 yl]methyl]-2-azaspiro[3.3]heptane-2-carboxylate

To a solution of tert-butyl 6-[[4-[1-(trifluoromethyl)vinyl]pyrazol-1-yl]methyl]-2-azaspiro[3.3]heptane-2-carboxylate (1000 mg, 2.69 mmol) in THE (20 mL) was added diphenyl(methyl)sulfonium tetrafluoroborate (1008 mg, 3.5 mmol). The suspension was cooled to 0° C. and NaHMDS/THF (1 M) (10.8 mL, 10.8 mmol) was added dropwise. The reaction mixture was warmed to 20° C. for and stirred for 12 h. The reaction mixture was purified by prep-HPLC and lyophilized to give the title compound (432 mg, 1.12 mmol, 42% yield) as light yellow solid. MS (ESI): m/z=386.1 [M+H]⁺

In analogy to Example B.377, the following building block was generated using the relevant commercial building blocks.

| | | | Building | MS, ESI: |
|---|---|---|---|---|
| Ex. | Structure | Systematic Name | Blocks | m/z |
| B.384 | 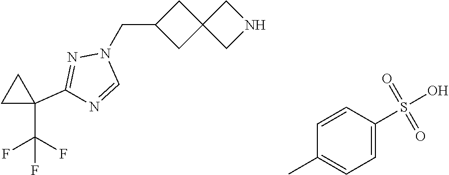 [SMILES: FC(F)(F)C1(c2ncn(CC3CC4(CNC4)C3)n2)CC1 ; Cc1ccc(S(=O)(=O)O)cc1] | 6-[[3-[1- (trifluoromethyl) cyclopropyl]- 1,2,4-triazol- 1-yl]methyl]-2- azaspiro[3.3]heptane; 4- methylbenzenesulfonic acid | 3-bromo-1H- 1,2,4-triazole | 287.0 [M − TsOH + H]⁺ |

### Example B.379

### 6-[[1-cyclopropyl-3-(trifluoromethyl)pyrazol-4-yl]methyl]-2-azaspiro[3.3]heptane; 4-methylbenzenesulfonic Acid

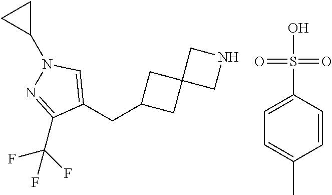

To the mixture of tert-butyl 6-[[1-cyclopropyl-3-(trifluoromethyl)pyrazol-4-yl]methyl]-2-azaspiro[3.3]heptane-2-carboxylate (790 mg, 2.05 mmol) in EtOAc (8 mL) was added p-toluenesulfonic acid (388 mg, 2.25 mmol) at 25° C., then the reaction mixture was stirred at 80° C. for 12 h. The reaction mixture was concentrated under reduced pressure to give a residue. 20 mL deionized water and 2 mL ACN was added to the residue, which was then lyophilized to give the title compound (811 mg, 1.77 mmol, 85% yield) as a yellow oil. MS (ESI): m/z=286.1 [M-TsOH+H]⁺

### Step a) Tert-Butyl 6-[[5-(trifluoromethyl)-1H pyrazol-4-yl]methylene]-2-azaspiro[3.3]heptane-2-carboxylate

To the solution of tert-butyl 6-[(4,4,5,5-tetramethyl-1,3,2-dioxaborolan-2-yl)methylene]-2-azaspiro[3.3]heptane-2-carboxylate (5009 mg, 14.9 mmol), 4-bromo-5-(trifluoromethyl)-1H-pyrazole (2920 mg, 13.6 mmol) in 1,4-dioxane (73 mL), water (14.6 mL) was added potassium carbonate (3750 mg, 27.2 mmol), 1,1′-bis(diphenylphosphino)ferrocene-palladium(II)dichloride dichloromethane complex (554 mg, 0.68 mmol) at 20° C., then the reaction was stirred at 100° C. for 12 h under N₂. The reaction mixture was filtered and concentrated under reduced pressure to give a residue which was purified by flash silica gel chromatography (eluent of 0-40% ethyl acetate/petroleum ether) to give a crude product which was further purified by flash silica gel chromatography (eluent of 0-40% ethyl acetate/petroleum ether) to give the title compound (790 mg, 2.3 mmol, 17% yield) as a yellow oil. MS (ESI): m/z=288.0 [M-C₄H₈+H]⁺.

### Step b) Tert-Butyl 6-[[1-cyclopropyl-3-(trifluoromethyl)pyrazol-4-yl]methylene]-2-azaspiro[3.3]heptane-2-carboxylate

To a mixture of tert-butyl 6-[[5-(trifluoromethyl)-1H-pyrazol-4-yl]methylene]-2-azaspiro[3.3]heptane-2-carboxylate (600 mg, 1.75 mmol), cyclopropylboronic acid (600 mg, 6.99 mmol) in DCE (6 mL) was added pyridine (0.42 mL, 5.24 mmol), boric acid (108 mg, 1.75 mmol), copper diacetate (698 mg, 3.5 mmol) at 20° C., then the reaction mixture was stirred at 100° C. for 16 h under O₂ (balloon). The reaction mixture was filtered and then diluted with water 50 mL and extracted with EtOAc 150 mL (50 mL×3). The combined organic layers were washed with brine (40 mL) dried over Na₂SO₄, filtered and concentrated under reduced pressure to give a residue which was purified by silica gel chromatography (0-28% ethyl acetate/petroleum ether) to give the title compound (350 mg, 0.91 mmol, 52%) as a colorless oil. MS (ESI): m/z=328.0 [M-C₄H₈+H]⁺.

### Step c) Tert-Butyl 6-[[1-cyclopropyl-3-(trifluoromethyl)pyrazol-4-yl]methyl]-2-azaspiro[3.3]heptane-2-carboxylate

To the mixture of tert-butyl 6-[[1-cyclopropyl-3-(trifluoromethyl)pyrazol-4-yl]methylene]-2-azaspiro[3.3]heptane-2-carboxylate (720 mg, 1.88 mmol) in EtOAc (15 mL) was added Pd/C (wet, 216 mg, 10%) at 25° C., then the reaction mixture was stirred at 25° C. for 0.5 h under H₂ (15 Psi). The reaction mixture was filtered and the filter liquor was concentrated under reduced pressure to give the title compound (640 mg, 1.66 mmol, 88.4% yield) as a colorless oil. MS (ESI): m/z=330.0 [M-C₄H₈+H]⁺.

### Example B.381

### 6-[[5-methyl-3-(trifluoromethyl)pyrazol-1-yl]methyl]-2-azaspiro[3.3]heptane; 4-methylbenzenesulfonic Acid

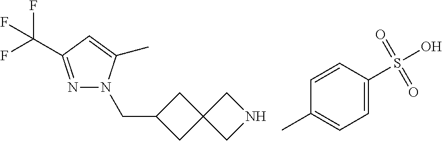

A mixture of tert-butyl 6-[[5-methyl-3-(trifluoromethyl)pyrazol-1-yl]methyl]-2-azaspiro[3.3]heptane-2-carboxylate (3.8 g, 10.57 mmol, 1.0 eq) and p-toluenesulfonic acid (4.55 g, 26.43 mmol, 2.5 eq) in EtOAc (70 mL) was stirred at 25° C. for 24 h. Then the reaction mixture was concentrated and crystallized from MTBE to give 4-methylbenzenesulfonic acid; 6-[[5-methyl-3-(trifluoromethyl)pyrazol-1-yl]methyl]-2-azaspiro[3.3]heptane (3940 mg, 9.13 mmol, 86 O/yield) as white solid. MS (ESI): m/z=260.2 [M+H]⁺

### Step a) Tert-Butyl 6-[[5-(methyl-3-(trifluoromethyl)pyrazol-1-yl]methyl]-2-azaspiro[3.3]heptane-2-carboxylate

To a solution of tert-butyl 6-(hydroxymethyl)-2-azaspiro[3.3]heptane-2-carboxylate (1000 mg, 4.4 mmol) in toluene (20 mL) was added 3-methyl-5-(trifluoromethyl)-1H-pyrazole (CAS: 10010-93-2) (660 mg, 4.4 mmol), and (Cyanomethylene)tributylphosphorane (1590 mg, 6.6 mmol). The reaction mixture was stirred at 100° C. for 12 h under N₂ atmosphere. The reaction mixture was filtered, and the filtrate was concentrated under reduced pressure to give a residue. The residue was purified by prep-HPLC and lyophilized to give the title compound (485 mg, 1.35 mmol, 31% yield) as dark brown powder. MS (ESI): m/z=304.0 [M+H]⁺

Note: Regioisomer tert-butyl 6-[[3-methyl-5-(trifluoromethyl)pyrazol-1-yl]methyl]-2-azaspiro[3.3]heptane-2-carboxylate (735 mg, 2.05 mmol, 46.5% yield) was also generated as a dark brown oil. MS (ESI): m/z=304.0 [M+H]⁺

### Example B.382

### 6-[[3-methyl-5-(trifluoromethyl)pyrazol-1-yl]methyl]-2-azaspiro[3.3]heptane; 4-methylbenzenesulfonic Acid

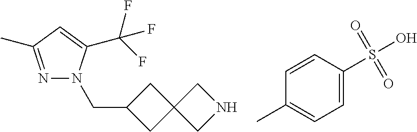

A mixture of tert-butyl 6-[[3-methyl-5-(trifluoromethyl)pyrazol-1-yl]methyl]-2-azaspiro[3.3]heptane-2-carboxylate (generated as regioisomer in B.381, Step a)) (2.3 g, 6.4 mmol) and p-toluenesulfonic acid (2755 mg, 16.0 mmol) in EtOAc (50 mL) was stirred at 25° C. for 24 h. Then the reaction mixture was concentrated and crystallized from MTBE to give the title compound (1468 mg, 3.4 mmol, 53% yield) as white solid. MS (ESI): m/z=260.2 [M+H]⁺

In analogy to Example B.381B.382, the following regioisomeric pairs of building blocks were generated using the relevant commercial building blocks.

| | | | | MS, |
|---|---|---|---|---|
| | | | | ESI: |
| Ex. | Structure | Systematic Name | Building Blocks | m/z |
| B.392 | 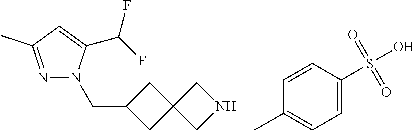 | 6-[[5- (difluoromethyl)-3- methyl-pyrazol-1- yl]methyl]-2- azaspiro[3.3]heptane; 4- methylbenzenesulfonic acid | 5- (difluoromethyl)- 3-methyl-1H- pyrazole (CAS: 936033-61-3) | 242.1 [M − TsOH + H]⁺ |
| B.393 | 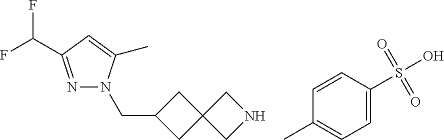 | 6-[[3- (difluoromethyl)-5- methyl-pyrazol-1- yl]methyl]-2- azaspiro[3.3]heptane; 4- methylbenzenesulfonic acid | 5- (difluoromethyl)- 3-methyl-1H- pyrazole (CAS: 936033-61-3) | 242.1 [M − TsOH + H]⁺ |
| B.411 | 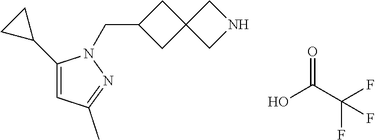 [SMILES: Cc1cc(C2CC2)n(CC2CC3(CNC3)C2)n1 ; O=C(O)C(F)(F)F] | 6-[(5-cyclopropyl-3- methyl-pyrazol-1- yl)methyl]-2- azaspiro[3.3]heptane; 2,2,2-trifluoroacetic acid | 5-cyclopropyl-3- methyl-1H- pyrazole (CAS: 1343588-67-9) | 232.2 [M − TFA + H]⁺ |
| | *mixture with B.412, | | | |
| | separation possible after | | | |
| | urea coupling | | | |
| B.412 | 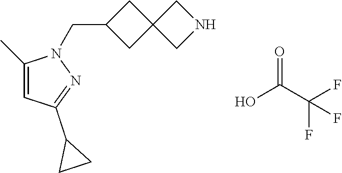 [SMILES: Cc1cc(C2CC2)nn1CC1CC2(CNC2)C1 ; O=C(O)C(F)(F)F] | 6-[(3-cyclopropyl-5- methyl-pyrazol-1- yl)methyl]-2- azaspiro[3.3]heptane; 2,2,2-trifluoroacetic acid | 5-cyclopropyl-3- methyl-1H- pyrazole (CAS: 1343588-67-9) | 232.2 [M − TFA + H]⁺ |
| | *mixture with B.411, | | | |
| | separation possible after | | | |
| | urea coupling | | | |

### Example B.386

### [4-(2-azaspiro[3.3]heptan-6-yloxy)phenyl]-imino-oxo-(trifluoromethyl)-λ⁶-sulfane; 4-methylbenzenesulfonic Acid

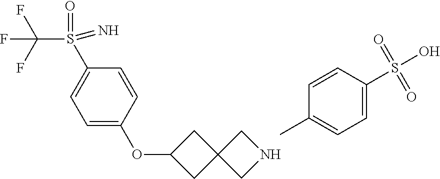

To solution of tert-butyl 6-[4-(trifluoromethylsulfonimidoyl)phenoxy]-2-azaspiro[3.3]heptane-2-carboxylate (380 mg, 0.9 mmol) in EtOAc (5 mL) was added p-toluenesulfonic acid monohydrate (206 mg, 1.08 mmol) and stirred at 25° C. for 18 h, then reaction mixture was evaporated and purified by HPLC to give the title compound (143 mg, 0.29 mmol, 31% yield) as a yellow solid. MS (ESI): m/z=321.0 [M+H]⁺

### Step a) 4-(trifluoromethylsulfonimidoyl)phenol

To a stirred solution of imino-oxo-[4-(4,4,5,5-tetramethyl-1,3,2-dioxaborolan-2-yl)phenyl]-(trifluoromethyl)-λ6-sulfane (CAS: 1798336-50-1) (1.5 g, 4.48 mmol) in THE (75 mL) at 0° C. under air was added sodium hydrogen carbonate (376 mg, 4.48 mmol) in water (7.5 mL), followed by slow addition of hydrogen peroxide (1522 mg, 44.8 mmol). The reaction mixture was stirred at room temperature for 4 h. The resultant mixture was washed with water and aqueous sodium hydrogensulfite before being dried and concentrated to afford 4-(trifluoromethylsulfonimidoyl)phenol (1.0 g, 4.44 mmol, 94% yield) as yellow solid. MS (ESI): m/z=226.0 [M+H]⁺

### Step b) Tert-Butyl 6-[4-(trifluoromethylsulfonimidoyl)phenoxy]-2-azaspiro[3.3]heptane-2-carboxylate

To a mixture of 4-(trifluoromethylsulfonimidoyl)phenol (1.0 g, 4.44 mmol) and tert-butyl 6-hydroxy-2-azaspiro[3.3]heptane-2-carboxylate (947 mg, 4.44 mmol) was added cyanomethylenetributylphosphorane (2140 mg, 8.88 mmol) in Toluene (30 mL), and the resulting mixture was heated at 120° C. and stirred for 48 h. The reaction solution was concentrated. The residue was purified by flash chromatography (hexane/MTBE (10-100%)) to obtain the title compound (320 mg, 0.76 mmol, 16% yield) as yellow solid. MS (ESI): m/z=365.0 [M-C₄H₈+H]⁺.

In analogy to Example B.386, the following building block was generated using the relevant commercial building blocks.

| | | | | MS, |
|---|---|---|---|---|
| | | | | ESI: |
| Ex. | Structure | Systematic Name | Building Blocks | m/z |
| B.390 | 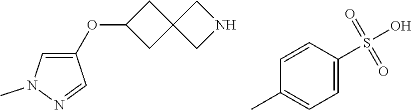 | 6-(1-methylpyrazol-4- yl)oxy-2- azaspiro[3.3]heptane; 4- methylbenzenesulfonic acid | 1-methylpyrazol- 4-ol | 194.2 [M − TsOH + H]⁺ |

### Example B.400

### 3-(2-azaspiro[3.3]heptan-6-ylmethyl)-5-(trifluoromethyl)-1,2,4-oxadiazole; 4-methylbenzenesulfonic Acid

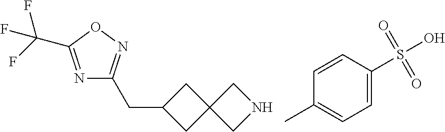

A mixture of tert-butyl 6-[[5-(trifluoromethyl)-1,2,4-oxadiazol-3-yl]methyl]-2-azaspiro[3.3]heptane-2-carboxylate (350 mg, 1.01 mmol) and TsOH (208 mg, 1.21 mmol) in Ethyl acetate (10 mL) was stirred at 80° C. for 12 h. The reaction mixture was concentrated under reduced pressure to give a residue. The residue was dissolved in MeCN (2.0 mL), water (20.0 mL) and lyophilized to afford the title compound (410 mg, 0.98 mmol, 91% yield) as a white solid. MS (ESI): m/z=248.0 [M-TsOH+H]⁺

### Step a) Tert-Butyl 6-[(2Z)-2-amino-2-hydroxyimino-ethyl]-2-azaspiro[3.3]heptane-2-carboxylate

To a mixture of hydroxylamine; hydrochloride (2353 mg, 33.9 mmol) in ethanol (25 mL) was added TEA (3426 mg, 33.9 mmol) at 25° C. After 1 h, tert-butyl 6-(cyanomethyl)-2-azaspiro[3.3]heptane-2-carboxylate (B.349, Step a)) (4000 mg, 16.9 mmol) was added to the reaction mixture, then the reaction was stirred at 50° C. for further 12 h. The reaction was concentrated under reduced pressure to give a residue. The residue was dissolved in water (50 mL), extracted with ethyl acetate (50 mL×3), and the combined extracts were concentrated under reduced pressure to give the title compound (4.5 g, 16.7 mmol. 99% yield) as a colorless oil, which was used for next step without further purification. MS (ESI): m/z=270.1 [M+H]⁺

### Step b) Tert-Butyl 6-[[5-(trifluoromethyl)-1,2,4-oxadiazol-3-yl]methyl]-2-azaspiro[3.3]heptane-2-carboxylate

To a solution of tert-butyl 6-[(2Z)-2-amino-2-hydroxyimino-ethyl]-2-azaspiro[3.3]heptane-2-carboxylate (800 mg, 2.97 mmol) in Methanol (20 mL) was added NaOMe (MeOH solution) (1.19 mL, 5.94 mmol) and ethyl trifluoroacetate (2110 mg, 14.9 mmol) at 30° C. Then the reaction was stirred at 50° C. for 12 h. The reaction was concentrated under reduced pressure to give a residue. The residue was dissolved in water (20 mL), the reaction mixture was extracted with ethyl acetate (10 mL×3), and the combined extracts were concentrated under reduced pressure to give a residue. The residue was purified by prep-HPLC and lyophilized to afford the title compound (360.0 mg, 1.04 mmol, 34.9% yield) as a colorless oil. MS (ESI): m/z=292.0 [M-C₄H₈+H]⁺

### Example B.408

### 6-[[1-(2,2,2-trifluoroethyl)-3-(trifluoromethyl)pyrazol-4-yl]methyl]-2-azaspiro[3.3]heptane; 4-methylbenzenesulfonic Acid

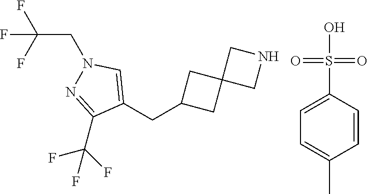

To the mixture of tert-butyl 6-[[1-(2,2,2-trifluoroethyl)-3-(trifluoromethyl)pyrazol-4-yl]methyl]-2-azaspiro[3.3]heptane-2-carboxylate (1.05 g, 2.46 mmol) in EtOAc (10 mL) was added p-toluenesulfonic acid (465 mg, 2.7 mmol) at 20° C., then the reaction mixture was stirred at 80° C. for 12 h. The reaction mixture was concentrated under reduced pressure to give a residue. Then to the residue was added 20 mL deionized water and 2 mL CAN. The mixture was lyophilized to give the title compound (1224 mg, 2.45 mmol, 99% yield) as a light yellow oil. MS (ESI): m/z=328.0 [M+H]⁺.

### Step a) Tert-Butyl 6-[[1-(2,2,2-trifluoroethyl)-3-(trichloromethyl)pyrazol-4-yl]methylene]-2-azaspiro[3.3]heptane-2-carboxylate

To the mixture of tert-butyl 6-[[5-(trifluoromethyl)-1H-pyrazol-4-yl]methylene]-2-azaspiro[3.3]heptane-2-carboxylate (B.379, Step a) (2000 mg, 5.83 mmol), cesium carbonate (5694 mg, 17.5 mmol) in DMF (20 mL) was added a solution of 2,2,2-trifluoroethyl trifluoromethanesulfonate (2704 mg, 11.7 mmol) in DMF (2 mL) at 0° C., and the mixture was stirred at 20° C. for 12 h. The reaction mixture was diluted with water (100 mL) and extracted with EtOAc (100 mL×3). The combined organic layers were washed with brince (50 mL) dried over Na₂SO₄, filtered and concentrated under reduced pressure to give a residue, which was purified by flash silica gel chromatography (Eluent of 0-28°/o Ethyl acetate/Petroleum ether) to give the title compound (1.1 g, 2.59 mmol, 44% yield) as a white solid. MS (ESI): m/z=370.0 [M-C₄H₈+H]⁺.

### Step b) Tert-Butyl 6-[[1-(2,2,2-trifluoroethyl)-3-(trifluoromethyl)pyrazol-4-yl]methyl]-2-azaspiro[3.3]heptane-2-carboxylate

To the mixture of tert-butyl 6-[[1-(2,2,2-trifluoroethyl)-3-(trifluoromethyl)pyrazol-4-yl]methylene]-2-azaspiro[3.3]heptane-2-carboxylate (1000 mg, 2.35 mmol) in EtOAc (10 mL) was added Pd/C (300 mg, 10%, wet.) at 20° C., then the reaction mixture was stirred at 20° C. for 1 h under H₂. The reaction mixture was concentrated under reduced pressure to give tert-butyl 6-[[1-(2,2,2-trifluoroethyl)-3-(trifluoromethyl)pyrazol-4-yl]methyl]-2-azaspiro[3.3]heptane-2-carboxylate (1.05 g, 2.46 mmol, 104% yield) as a colorless oil. MS (ESI): m/z=372.0 [M-C₄H₈+H]⁺.

### Example B.409

### 6-[(3-cyclopropyl-1H-pyrazol-5-yl)methyl]-2-azaspiro[3.3]heptane; 2,2,2-trifluoroacetic Acid

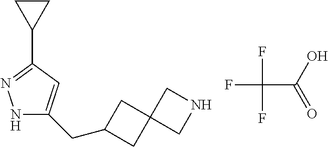

To a solution of 6-[(3-cyclopropyl-1H-pyrazol-5-yl)methyl]-2-azaspiro[3.3]heptane-2-carboxylic acid tert-butyl ester (180 mg, 567 μmol) in dichloromethane (2.44 mL) was added TFA (647 mg, 437 μL, 5.67 mmol) and the reaction mixture was stirred at RT for 18 h. Volatiles were removed in vacuo to give 374 mg of the crude title compound which was used without further purification, purity approx. 50% major contaminant excess of TFA. MS (ESI): m/z=218.2 [M-TFA+H]⁺.

### Step a) Tert-Butyl 6-(4-cyclopropyl-2,4-dioxo-butyl)-2-azaspiro[3.3]heptane-2-carboxylate

To a solution of tert-butyl 6-(2-ethoxy-2-oxo-ethyl)-2-azaspiro[3.3]heptane-2-carboxylate (CAS: 2173992-27-) (2000 mg, 7.06 mmol) in THE (20 mL) was added NaH (791 mg, 19.8 mmol) at 0° C. After 0.5 h, cyclopropyl methyl ketone (594 mg, 7.06 mmol) was added at 0° C. The mixture was stirred at 60° C. for 12 h. The reaction mixture was quenched by addition H₂O (10 mL) at 25° C., and then diluted with H₂O (10 mL) and extracted with EtOAc (10 mL×3). The combined organic layers were washed with sat. aq. NaCl solution (10 mL×3), dried over anhydrous Na₂SO₄, filtered and concentrated under reduced pressure to give a residue. The residue was purified by prep-TLC (SiO₂, Petroleum ether/Ethyl acetate=3:1) to give the title compound (600 mg, 1.87 mmol, 26% yield) as a yellow oil. MS (ESI): m/z=266.2 [M-C₄H₈+H]⁺.

### Step b) Tert-Butyl 6-[(5-cyclopropyl-1H-pyrazol-3 yl)methyl]-2-azaspiro[3.3]heptane-2-carboxylate

To a solution of tert-butyl 6-(4-cyclopropyl-2,4-dioxo-butyl)-2-azaspiro[3.3]heptane-2-carboxylate (540 mg, 1.68 mmol) in Ethanol (5 mL) was added hydrazine (108 mg, 3.36 mmol) at 20° C. and the resulting mixture was stirred at 60° C. for 1 h. The filter liquor was purified by prep-HPLC and lyophilized to give the title compound (458 mg, 1.44 mmol, 77% yield) as colorless oil. MS (ESI): m/z=262.1 [M-C₄H₈+H]⁺.

### Example B.414

### 5-(2-azaspiro[3.3]heptan-6-ylmethyl)-3-(trifluoromethyl)-1,2,4-oxadiazole; 4-methylbenzenesulfonic Acid

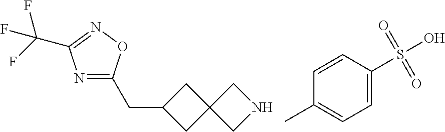

To a solution of tert-butyl 6-[[3-(trifluoromethyl)-1,2,4-oxadiazol-5-yl]methyl]-2-azaspiro[3.3]heptane-2-carboxylate (118.0 mg, 0.34 mmol) in EtOAc (2 mL) was added p-toluenesulfonic acid (70.2 mg, 0.41 mmol) at 80° C. and stirred for 12 h. The mixture was concentrated under reduced pressure to give a residue. To the residue was added deionized water and lyophilized to give the title compound (125 mg, 0.3 mmol, 84% yield) as white solid. MS (ESI): m/z=248.1 [M-TsOH+H]⁺

### Step a) Tert-Butyl 6-[[3-(trifluoromethyl)-1,2,4-oxadiazol-5-yl]methyl]-2-azaspiro[3.3]heptane-2-carboxylate

To a solution of tert-butyl 6-(2-ethoxy-2-oxo-ethyl)-2-azaspiro[3.3]heptane-2-carboxylate (CAS: 2173992-27-1) (300 mg, 1.06 mmol), 2,2,2-trifluoro-N′-hydroxy-acetamidine (CAS: 4314-35-6) in methanol (3 mL) was added sodium methoxide in MeOH solution (5 M) (0.42 mL, 2.12 mmol) at 25° C., then the mixture was stirred at 60° C. for 12 h. The residue was purified by prep-HPLC and lyophilized to give the title compound (120 mg, 0.35 mmol, 33% yield) as yellow oil. ¹H NMR (400 MHz, CHLOROFORM-d) δ=3.96 (s, 2H), 3.85 (s, 2H), 3.06 (d, J=7.6 Hz, 2H), 2.71 (spt, J=7.9 Hz, 1H), 2.51-2.39 (m, 2H), 2.07-1.95 (m, 2H), 1.44 (s, 9H). MS (ESI): m/z=292.0 [M-C₄H₈+H]⁺;

### Example B.415

### 6-[[2-(2,2,2-trifluoroethyl)-5-(trifluoromethyl)pyrazol-3-yl]methyl]-2-azaspiro[3.3]heptane; 4-methylbenzenesulfonic Acid

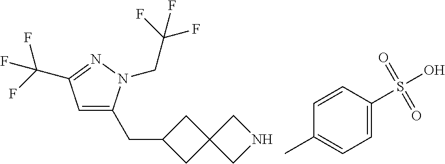

A solution of p-toluenesulfonic acid (247 mg, 1.43 mmol) and tert-butyl 6-[[2-(2,2,2-trifluoroethyl)-5-(trifluoromethyl)pyrazol-3-yl]methyl]-2-azaspiro[3.3]heptane-2-carboxylate (510 mg, 1.19 mmol) in EtOAc (5 mL) was stirred at 80° C. for 12 h. The mixture was concentrated under reduced pressure to give residue. To the residue was added deionized water and lyophilized to give the title compound (550 mg, 1.1 mmol. 92.3% yield) as a colorless oil. MS (ESI): m/z=328.0 [M-TsOH+H]⁺

### Step a) Tert-Butyl 6-[[2-(2,2,2-trifluoroethyl)-5-(trifluoromethyl)pyrazol-3-yl]methyl]-2-azaspiro[3.3]heptane-2-carboxylate

To a solution of tert-butyl 6-[[5-(trifluoromethyl)-1H-pyrazol-3-yl]methyl]-2-azaspiro[3.3]heptane-2-carboxylate (generated in the synthesis of P.7, Step c)) (1.5 g, 4.34 mmol) in THE (15 mL) was added NaH (261 mg, 6.51 mmol) at 0° C. for 0.5 h. Then 2,2,2-trifluoroethyl trifluoromethanesulfonate (2016 mg, 8.69 mmol) was added at 20° C., and the reaction stirred for 12 h. The reaction was quenched by ice slowly and then extracted with EtOAc (20 mL×3). The combined organic phase was washed with brine (20 mL×3), dried over anhydrous Na₂SO₄, filtered and concentrated in vacuo. The residue was purified by prep-HPLC and lyophilized to give regioisomer tert-butyl 6-[[1-(2,2,2-trifluoroethyl)-5-(trifluoromethyl)pyrazol-3-yl]methyl]-2-azaspiro[3.3]heptane-2-carboxylate (240.0 mg, 0.56 mmol, 12.93% yield) (crude) and tert-butyl 6-[[2-(2,2,2-trifluoroethyl)-5-(trifluoromethyl)pyrazol-3-yl]methyl]-2-azaspiro[3.3]heptane-2-carboxylate (800 mg, 1.87 mmol, 43.1% yield) (crude) as yellow solid. The residue was purified by prep-HPLC to give the title compound (530 mg, 1.24 mmol, 66% yield) as a colorless oil. MS (ESI): m/z=372.0 [M-C₄H₈+H]⁺

### Example B.416

### 6-[[1-(2,2,2-trifluoroethyl)-5-(trifluoromethyl)pyrazol-3-yl]methyl]-2-azaspiro[3.3]heptane; 4-methylbenzenesulfonic Acid

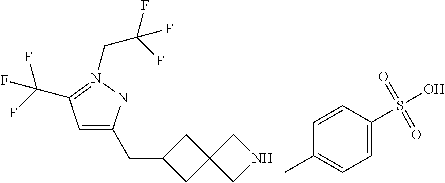

A solution of p-toluenesulfonic acid (43.5 mg, 0.25 mmol) and tert-butyl 6-[[1-(2,2,2-trifluoroethyl)-5-(trifluoromethyl)pyrazol-3-yl]methyl]-2-azaspiro[3.3]heptane-2-carboxylate (regioisomeric product from B.415, Step a)) (90.0 mg, 0.21 mmol) in EtOAc (2 mL) was stirred at 80° C. for 12 h. The mixture was concentrated under reduced pressure to give residue. The residue was added deionized water and lyophilized to give 4-methylbenzenesulfonic acid; 6-[[1-(2,2,2-trifluoroethyl)-5-(trifluoromethyl)pyrazol-3-yl]methyl]-2-azaspiro[3.3]heptane (95.4 mg, 0.19 mmol, 88.31% yield) as a off-white solid. MS (ESI): m/z=328.0 [M-TsOH+H]⁺; ¹H NMR (400 MHz, DMSO-d6) δ=8.56-8.34 (m, 1H), 7.47 (d, J=8.1 Hz, 3H), 7.11 (d, J=7.9 Hz, 3H), 6.85 (s, 1H), 5.16 (q, J=8.8 Hz, 2H), 4.00-3.92 (m, 2H), 3.87-3.79 (m, 2H), 2.64 (d, J=7.3 Hz, 2H), 2.48-2.35 (m, 2H), 2.34-2.30 (m, 1H), 2.29 (s, 4H), 1.95-1.86 (m, 2H)

### Example B.443

### 6-[[3-(2,2,2-trifluoroethyl)-1H-1,2,4-triazol-5-yl]methyl]-2-azaspiro[3.3]heptane; 2,2,2-trifluoroacetic Acid

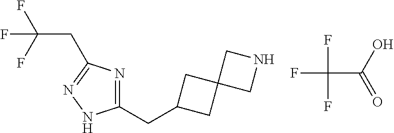

To a solution of 6-[[5-(2,2,2-trifluoroethyl)-1H-1,2,4-triazol-3-yl]methyl]-2-azaspiro[3.3]heptane-2-carboxylic acid tert-butyl ester (155 mg, 430 μmol) in dichloromethane (2 mL) was added TFA (490 mg, 331 μL, 4.3 mmol) and the reaction mixture was stirred at RT for 18 h. Volatiles were removed in vacuo to yield 31 8 mg of the crude title compound as a viscous yellow oil, purity ˜50% major contaminant excess of TFA, which was used without further purification. MS (ESI): m/z=261.2 [M-TFA+H]⁺

### Step a) 6-[[5-(2,2,2-trifluoroethyl)-1H-1,2,4-triazol-3 yl]methyl]-2-azaspiro[3.3]heptane-2-carboxylic Acid Tert-Butyl Ester

To a solution of 3,3,3-trifluoropropanehydrazide (CAS: 934171-99-0) (272 mg, 1.91 mmol) and tert-butyl 6-(2-amino-2-imino-ethyl)-2-azaspiro[3.3]heptane-2-carboxylate; acetic acid (B.349, Step c)) (600 mg, 1.91 mmol) in DMF (5 mL) was added EtONa (195 mg, 2.87 mmol) at 25b° C. under N₂ atmosphere. Then the reaction was stirred at 100° C. under N₂ atmosphere for 12 h. The reaction was purified by prep-HPLC and lyophilized to afford the title compound (200 mg, 0.55 mmol, 29% yield) as a white solid. MS (ESI): m/z=361.1 [M+H]⁺; ¹H NMR (400 MHz, METHANOL-d4) δ=3.92 (s, 2H), 3.80 (s, 2H), 3.67-3.50 (m, 2H), 2.84 (br d, J=7.2 Hz, 2H), 2.57 (td, J=7.9, 15.6 Hz, 1H), 2.31 (ddd, J=2.4, 8.0, 10.4 Hz, 2H), 1.98-1.91 (m, 2H), 1.42 (s, 9H)

### Example B.493

### 6-[6-(trifluoromethyl)-3-pyridyl]-2-azaspiro[3.3]heptane; 4-methylbenzenesulfonic Acid

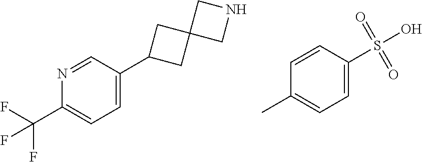 [SMILES: FC(F)(F)c1ccc(C2CC3(CNC3)C2)cn1 ; Cc1ccc(S(=O)(=O)O)cc1]

The mixture of tert-butyl 6-[6-(trifluoromethyl)-3-pyridyl]-2-azaspiro[3.3]heptane-2-carboxylate (1450 mg, 4.24 mmol) and p-toluenesulfonic acid (1605 mg, 9.32 mmol) in ethyl acetate (10 mL) was stirred at 80° C. for 16 h. The reaction mixture was filtered and the cake was concentrated to give the title compound (2110 mg, 3.6 mmol, 84% yield) as a off-white solid. MS (ESI): m/z=243.3 [M-TsOH+H]⁺

### Step a) Tert-Butyl 6-[6-(trifluoromethyl)-3-pyridyl]-2-azaspiro[3.3]heptane-2-carboxylate

Two batches were set up in parallel. Tert-butyl 6-iodo-2-azaspiro[3.3]heptane-2-carboxylate (1.0 g, 3.09 mmol), 2-trifluoromethylpyridine-5-boronic acid (1180 mg, 6.19 mmol), Sodium bis(trimethylsilyl)amide in THE (6.19 mL, 6.19 mmol), trans-2-aminocyclohexanol hydrochloride (28.2 mg, 0.190 mmol) and nickel(II) iodide (58.0 mg, 0.190 mmol) were taken up into a microwave tube in 2-propanol (10 mL). The sealed tube was heated at 110° C. for 2.5 h under microwave. The reaction was quenched by H₂O slowly. The residue was purified by flash silica gel chromatography (eluent of 0 to 20% ethyl acetate/petroleum ether gradient) to give the title compound (1.5 g, 4.38 mmol, 71% yield) as yellow solid. MS (ESI): m/z=287.2 [M-tBu+H]⁺

In analogy to Example B.493, the following building block was generated using the relevant commercial building blocks.

| | | | | MS, |
|---|---|---|---|---|
| | | | | ESI: |
| Ex. | Structure | Systematic Name | Building Blocks | m/z |
| B.538 | 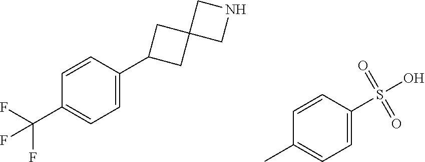 [SMILES: FC(F)(F)c1ccc(C2CC3(CNC3)C2)cc1 ; Cc1ccc(S(=O)(=O)O)cc1] | 6-[4- (trifluoromethyl) phenyl]-2- azaspiro[3.3]heptane; 4- methylbenzenesulfonic acid | 4- (trifluoromethyl) phenyl]boronic acid | 242.1 [M − TsOH + H]⁺ |

### Example B.494

### 6-(3-cyclopropyl-1H-1,2,4-triazol-1-yl)-2-azaspiro[3.3]heptane 2,2,2-trifluoroacetate

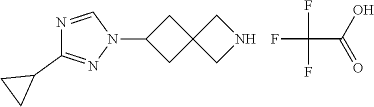

To a solution of tert-butyl 6-(3-cyclopropyl-1,2,4-triazol-1-yl)-2-azaspiro[3.3]heptane-2-carboxylate (6.00 g, 19.7 mmol) in DCM (120 mL) was added TFA (46.2 g, 405 mmol, 30 mL) at 25° C. The mixture was stirred at 30° C. for 16 h. The reaction mixture was filtered and concentrated under reduced pressure to give a residue. The crude product was used into the next step without further purification. The title compound (14.0 g, crude) was used in the next step without further purification. MS (ESI): m/z=205.2 [M+H]⁺

### Step a) Tert-Butyl 6-methylsulfonyloxy-2-azaspiro[3.3]heptane-2-carboxylate

To a solution of tert-butyl 6-hydroxy-2-azaspiro[3.3]heptane-2-carboxylate (CAS: 1147557-97-8) (10.0 g, 46.9 mmol) in DCM (200 mL) was added TEA (7.12 g, 70.3 mmol, 9.79 mL) and MsCl (6.90 g, 60.2 mmol, 4.66 mL) dropwise at 0° C., The mixture was stirred at 30° C. for 2 h. The reaction mixture was quenched by addition of aq NaHCO₃ solution (200 mL), and then extracted with DCM (300 mL×2). The combined organic layers were dried over Na₂SO₄, filtered and concentrated under reduced pressure to give a residue (13.5 g crude, 46.3 mmol, 98.8% yield), which was used into the next step without further purification. MS (ESI): m/z=236.2 [M+H]⁺

### Step b) Tert-Butyl 6-(3-cyclopropyl-1.2,4-triazol-1 yl)-2-azaspiro[3.3]heptane-2-carboxylate

To a solution of tert-butyl 6-methylsulfonyloxy-2-azaspiro[3.3]heptane-2-carboxylate (12.0 g, 41.2 mmol, 90.0% purity) in ACN (200 mL) was added 3-cyclopropyl-1H-1,2,4-triazole (CAS: 1211390-33-8) (4.50 g, 41.2 mmol) and Cs₂CO₃ (26.8 g, 82.4 mmol) at 25° C. The mixture was stirred at 100° C. for 16 h. The reaction mixture was filtered and concentrated under reduced pressure to give a residue. The residue was further separated by SFC to obtain the title compound (6.77 g, 22.2 mmol, 54.0% yield) as a brown solid. MS (ESI): m/z=305.2 [M+H]⁺

### Example B.504

### [6-(2-azaspiro[3.3]heptan-6-ylmethyl)-3-pyridyl]-imino-oxo-(trifluoromethyl)-λ6-sulfane; 4-methylbenzenesulfonic Acid

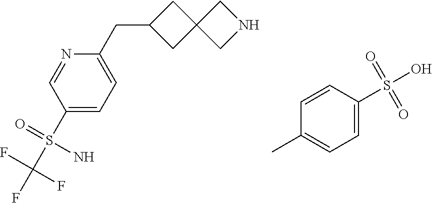

To a solution of tert-butyl 6-[[5-(trifluoromethylsulfonimidoyl)-2-pyridyl]methyl]-2-azaspiro[3.3]heptane-2-carboxylate (4.8 g, 11.4 mmol) in EtOAc (48 mL) was added p-toluenesulfonic acid (2.36 g, 13.7 mmol) at 20° C. Then the mixture was stirred at 80° C. for 12 h. The mixture was concentrated to remove the solvent, then deionized water was added and mixture was lyophilized to give the title compound (5608 mg, 11.4 mmol, 93.8% yield) as a brown solid. MS (ESI): m/z=320.0 [M-TsOH+H]⁺

### Step a) Tert-Butyl 6-[[5-(trifluoromethylsulfanyl)-2-pyridyl]methylene]-2-azaspiro[3.3]heptane-2-carboxylate

To a solution of tert-butyl 6-[(4,4,5,5-tetramethyl-1,3,2-dioxaborolan-2-yl)methylene]-2-azaspiro[3.3]heptane-2-carboxylate (58.8 g, 175 mmol), 2-chloro-5-(trifluoromethylsulfanyl)pyridine (CAS: 1204234-95-6) (37.5 g, 175 mmol) and K₂CO₃ (48.5 g, 351 mmol) in 1,4-Dioxane (400 mL) and water (80 mL) was added Pd(dppf)Cl₂·DCM (14.3 g, 17.5 mmol) at 20° C., the mixture was stirred at 80° C. under N₂ atmosphere for 12 h. The reaction mixture was filtered and the filtrate was concentrated under vacuum to give a crude product which was purified by silica gel chromatography (eluent of 0-30% Ethyl acetate/Petroleum ether) and concentrated under reduced pressure to give the title compound (28500 mg, 73.8 mmol, 42.1% yield) as an off-white solid. MS (ESI): m/z=331.0 [M-C₄H₈+H]⁺

### Step b) Tert-Butyl 6-[[5-(trifluoromethylsulfanyl)-2 pyridyl]methyl]-2-azaspiro[3.3]heptane-2-carboxylate

To a solution of tert-butyl 6-[[5-(trifluoromethylsulfanyl)-2-pyridyl]methylene]-2-azaspiro[3.3]heptane-2-carboxylate (28.0 g, 72.5 mmol) in EtOAc (280 mL) was added wet Pd/C (28.0 g) at 30° C., then the mixture was stirred at 30° C. for 12 h. The reaction mixture was filtered and the filtrate was concentrated under vacuum to give tert-butyl 6-[[5-(trifluoromethylsulfanyl)-2-pyridyl]methyl]-2-azaspiro[3.3]heptane-2-carboxylate (27.0 g, 69.5 mmol, 96% yield) as yellow solid. MS (ESI): m/z=333.0 [M-C₄H₈+H]⁺

### Step c) Tert-Butyl 6-[[5-(trifluoromethylsuifonimidoyl)-2-pyridyl]methyl]-2-azaspiro[3.3]heptane-2-carboxylate

To a solution of tert-butyl 6-[[5-(trifluoromethylsulfanyl-2-pyridyl]methyl]-2-azaspiro[3.3]heptane-2-carboxylate (20.0 g, 51.5 mmol) in HFIP (300 mL) was added PhI(OAc)₂ (69.7 g, 216 mmol) and NH₂COONH₄ (12.1 g, 154 mmol) at 20° C. The mixture was stirred at 25° C. for 12 h. The mixture was concentrated under vacuum to give residue. The residue was purified by prep-HPLC (water (FA)-ACN) and lyophilized to give the title compound (4.0 g, 9.54 mmol, 18.5% yield) as a dark brown oil. MS (ES): m/z=364.1 [M-C₄H₈+H]⁺

In analogy to Example B.504, the following building block was generated using the relevant commercial building blocks.

| | | | | MS, |
|---|---|---|---|---|
| | | | | ESI: |
| Ex. | Structure | Systematic Name | Building Blocks | m/z |
| B.541 | 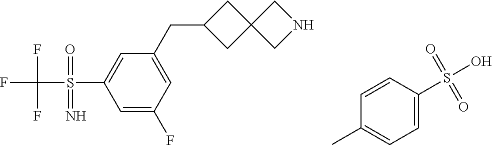 [SMILES: N=S(=O)(c1cc(F)cc(CC2CC3(CNC3)C2)c1)C(F)(F)F ; Cc1ccc(S(=O)(=O)O)cc1] | [3-(2- azaspiro[3.3]heptan-6- ylmethyl)-5-fluoro- phenyl]-imino-oxo- (trifluoromethyl)-λ⁶- sulfane; 4- methylbenzenesulfonic acid | 1-bromo-3- fluoro-5- (trifluoromethyl- sulfanyl)benzene (C.24) | 337.0 [M − TsOH + H]⁺ |

### Example B.520

### 2-[[6-(2,2,2-trifluoroethoxy)-3-pyridyl]methyl]-2,6-diazaspiro[3.3]heptane; 4-methylbenzenesulfonic Acid

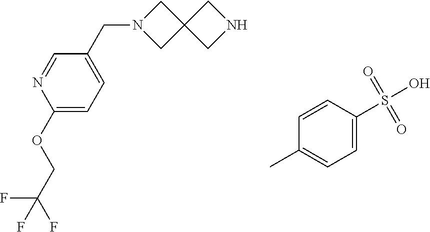 [SMILES: FC(F)(F)COc1ccc(CN2CC3(CNC3)C2)cn1 ; Cc1ccc(S(=O)(=O)O)cc1]

The mixture of tert-butyl 6-[[6-(2,2,2-trifluoroethoxy)-3-pyridyl]methyl]-2,6-diazaspiro[3.3]heptane-2-carboxylate (800.0 mg, 2.07 mmol) and p-toluenesulfonic acid monohydrate (1.3 g, 6.81 mmol) in EtOAc (60 mL) was stirred at 55° C. for 2 h. After cooling to RT, the obtained precipitate was filtered, washed with EtOAc and dried to give the title compound (1.1 g, 1.74 mmol, 80.1% yield) as white solid. MS (ESI): m/z=288.2 [M+H]⁺.

### Step a) Tert-Butyl 6-[[6-(2,2,2-trifluoroethoxy)-3 pyridyl]methyl]-2,6-diazaspiro[3.3]heptane-2-carboxylate

To a stirred suspension of tert-butyl 6-[(6-oxo-1H-pyridin-3-yl)methyl]-2,6-diazaspiro[3.3]heptane-2-carboxylate (B.519, Step a) (2.7 g, 8.84 mmol) and potassium carbonate (4.89 g, 35.4 mmol) in DMF (50 mL), 2,2,2-trifluoroethyl trifluoromethanesulfonate (3.08 g, 13.3 mmol) was added. Then RM was stirred at RT for 18 h The precipitate was filtered-off and filtrate was partitioned between water (150 mL) and TBME (250 mL). The organic layer was washed with brine, dried over Na₂SO₄ and evaporated in vacuum. The crude product was purified by flash chromatography (hexane/MTBE (50-100%)/methanol (0-100%)) to give two regioisomers: tert-butyl 6-[[6-oxo-1-(2,2,2-trifluoroethyl)-3-pyridyl]methyl]-2,6-diazaspiro[3.3]heptane-2-carboxylate (precursor of B.549) (750 mg, 1.94 mmol, 20.8% yield); MS (ESI): m/z=388.2 [M+H]⁺) and tert-butyl 6-[[6-(2,2,2-trifluoroethoxy)-3-pyridyl]methyl]-2,6-diazaspiro[3.3]heptane-2-carboxylate (1.1 g, 2.84 mmol, 30.51% yield); MS (ESI): m/z=388.0 [M+H]⁺).

In analogy to the sequence illustrated by B.519/B.520 the following building blocks were generated using the relevant commercial building blocks.

| | | | | MS, |
|---|---|---|---|---|
| | | | | ESI: |
| Ex. | Structure | Systematic Name | Building Blocks | m/z |
| B.521 | 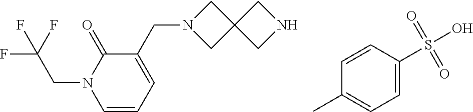 [SMILES: O=c1c(CN2CC3(CNC3)C2)cccn1CC(F)(F)F ; Cc1ccc(S(=O)(=O)O)cc1] | 3-(2,6- diazaspiro[3.3]heptan- 2-ylmethyl)-1-(2,2,2- trifluoroethyl)pyridin- 2-one; 4- methylbenzenesulfonic acid | 2- hydroxynicotin- aldehyde (CAS: 36404-89-4) | 288.2 [M − TsOH + H]⁺ |
| B.549 | 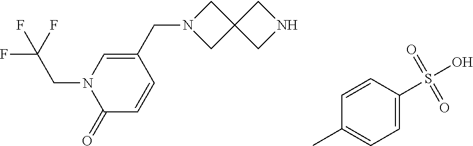 [SMILES: O=c1ccc(CN2CC3(CNC3)C2)cn1CC(F)(F)F ; Cc1ccc(S(=O)(=O)O)cc1] | 5-(2,6- diazaspiro[3.3]heptan- 2-ylmethyl)-1-(2,2,2- trifluoroethyl)pyridin- 2-one; 4- methylbenzenesulfonic acid | 6-oxo-1H- pyridine-3- carbaldehyde (CAS: 106984- 91-2) | 288.0 [M − TsOH + H]⁺ |

### Example B.526

### 5-(2,6-diazaspiro[3.3]heptan-2-ylmethyl)-3-[1-(trifluoromethyl)cyclopropyl]-1,2,4-oxadiazole; Trifluoroacetic Acid

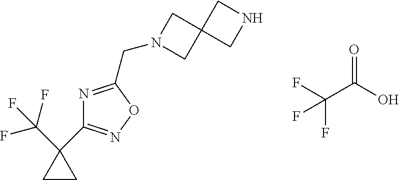 [SMILES: FC(F)(F)C1(c2noc(CN3CC4(CNC4)C3)n2)CC1 ; O=C(O)C(F)(F)F]

To a solution of 6-[[3-[1-(trifluoromethyl)cyclopropyl]-1,2,4-oxadiazol-5-yl]methyl]-2,6-diazaspiro[3.3]heptane-2-carboxylic acid tert-butyl ester (231 mg, 595 μmol) in dichloromethane (1.98 mL) was added TFA (678 mg, 458 μL, 5.95 mmol) and the reaction mixture was stirred at RI for 18 h. Volatiles were removed in vacuo to give 478 mg of the crude desired product as a light yellow viscous oil, purity ˜50% major contaminant excess of TFA. The crude was used without further purification. MS (ESI): m/z=289.2 [M+H]⁺

### Step a) Tert-Butyl 6-[2-[(Z)-[amino-[1-(trifluoromethyl)cyclopropyl]methylene]amino]oxy-2-oxo-ethyl]-2,6-diazaspiro[3.3]heptane-2-carboxylate

A solution of 2-(2-tert-butoxycarbonyl-2,6-diazaspiro[3.3]heptan-6-yl)acetic acid (CAS: 1937278-38-0) (1.45 g, 5.66 mmol) and CDI (1.1 g, 6.79 mmol) in DMF (10 mL) was stirred at room temperature for 3 hours. N′-hydroxy-1-(trifluoromethyl)cyclopropanecarboxamidine (CAS: 2172624-76-7) (951 mg, 5.66 mmol) was added to the solution and stirred at 30° C. for 12 h. The reaction was purified by prep-HPLC (water (NH₄HCO₃)-ACN 27%-57) to afford the title compound (670 mg, 1.65 mmol, 29% yield) as a white solid. MS (ESI): m/z=407.1 [M+H]⁺; ¹H NMR (400 MHz, CHLOROFORM-d) δ=5.26 (br s, 1H), 4.09 (s, 1H), 4.02 (s, 3H), 3.97 (br s, 1H), 3.54 (s, 4H), 3.48-3.39 (m, 2H), 1.44 (s, 9H), 1.40-1.10 (m, 4H)

### Step b) 6-[[3-[1-(trifluoromethyl)cyclopropyl]-1,2,4-oxadiazol-5 yl]methyl]-2,6-diazaspiro[3.3]heptane-2-carboxylic Acid Tert-Butyl Ester

A solution of tert-butyl 6-[2-[(Z)-[amino-[1-(trifluoromethyl)cyclopropyl]methylene]amino]oxy-2-oxo-ethyl]-2,6-diazaspiro[3.3]heptane-2-carboxylate (600 mg, 1.48 mmol) and Bu₄NF (0.37 mL, 0.37 mmol, 0.25 eq) in THE (15 mL) was stirred at 70° C. for 1 h. The reaction was purified by prep-TLC (PE: EtOAc=1:1) to the crude product. The crude product was purified by prep-HPLC (water (FA)-ACN 18°%-48%) to afford the title compound (260 mg, 0.67 mmol, 43% yield) as a yellow solid. MS (ESI): m/z=389.0 [M+H]⁺; ¹H NMR (400 MHz, CHLOROFORM-d) δ=4.00 (s, 4H), 3.81 (s, 2H), 3.52 (s, 4H), 1.52 (br d, J=3.3 Hz, 2H), 1.48 (br d, J=3.0 Hz, 2H), 1.42 (s, 9H).

### Example B.528

### 6-[(4-methylsulfonylpyrazol-1-yl)methyl]-2-azaspiro[3.3]heptane; 4-methylbenzenesulfonic Acid

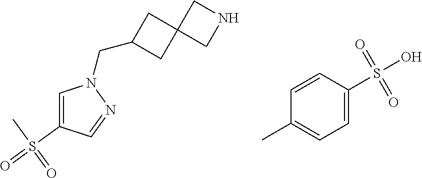 [SMILES: CS(=O)(=O)c1cnn(CC2CC3(CNC3)C2)c1 ; Cc1ccc(S(=O)(=O)O)cc1]

A mixture of tert-butyl 6-[(4-methylsulfonylpyrazol-1-yl)methyl]-2-azaspiro[3.3]heptane-2-carboxylate (1.29 g, 3.63 mmol) and p-toluenesulfonic acid (750 mg, 4.36 mmol) in EtOAc (50 mL) was stirred at 80° C. for 12 h under N₂ balloon. The reaction was concentrated and the residue was dissolved in water (30 mL). The solution was lyophilized to afford the title compound (1.46 g, 3.41 mmol, 90% yield) as a light yellow solid. MS (ESI): m/z=256.2 [M+H]⁺; ¹H NMR (400 MHz, CHLOROFORM-d) δ=8.58 (br s, 2H), 7.93 (s, 1H), 7.72 (s, 1H), 7.62 (d, J=8.0 Hz, 2H), 7.11 (d, J=8.0 Hz, 2H), 4.10-3.85 (m, 6H), 3.01 (s, 3H), 2.54 (dd, J=7.9, 15.6 Hz, 1H), 2.28 (s, 3H), 2.25-2.15 (m, 2H), 1.95-1.86 (m, 2H).

### Step a) Tert-Butyl 6-[(4-iodopyrazol-1 yl)methylene]-2-azaspiro[3.3]heptane-2-carboxylate

To a solution of tert-butyl 6-[(4,4,5,5-tetramethyl-1,3,2-dioxaborolan-2-yl)methylene]-2-azaspiro[3.3]heptane-2-carboxylate (45.6 g, 136 mmol), 4-iodopyrazole (24.0 g, 123 mmol), boric acid (7.65 g, 123 mmol) and pyridine (30 mL, 371 mmol) in DCE (230 mL) was added copper diacetate (49.4 g, 247 mmol) under O₂ atmosphere. The mixture was stirred at 70° C. for 12 h under 02 atmosphere. The reaction was concentrated. The residue was purified by flash chromatography on silica gel eluting with PE/EtOAc (4:1) to afford the crude product, which was purified by prep-HPLC (water(FA)-ACN 55%-75%) to afford the title compound (17.7 g, 44.1 mmol, 35.6% yield) as a white solid. MS (ESI): m/z=346.0 [M-C₄H₈+H]⁺

### Step b) Tert-Butyl 6-[(4-methylsulfonylpyrazol-1 yl)methylene]-2-azaspiro[3.3]heptane-2-carboxylate

To a solution of tert-butyl 6-[(4-iodopyrazol-1-yl)methylene]-2-azaspiro[3.3]heptane-2-carboxylate (2.0 g, 4.98 mmol, 1.0 eq) in DMSO (30 mL) was added sodium methanesulfonate (1.18 g, 9.97 mmol, 2.0 eq), Cu(OTf)₂ (361 mg, 1.0 mmol) and (R,R)-1,2-bis(N-methylamino)cyclohexane (142 mg, 1.0 mmol) at 110° C. for 12 h. The reaction was concentrated and the residue was purified by flash chromatography on silica gel eluting with PE/EtOAc (1:2) to afford the title compound (1.45 g, 4.1 mmol, 82% yield) as a white solid. MS (ESI): m/z=298.1 [M-C₄H₈+H]⁺

### Step c) Tert-Butyl 6-[(4-methylsulfonylpyrazol-1 yl)methyl]-2-azaspiro[3.3]heptane-2 carboxylate

To a solution of tert-butyl 6-[(4-methylsulfonylpyrazol-1-yl)methylene]-2-azaspiro[3.3]heptane-2-carboxylate (1.25 g, 3.54 mmol) in EtOAc (50 mL) was added Pd/C (0.5 g, 0.35 mmol) at 25° C. and stirred for 1 h under H₂ balloon (760 mm Hg). The reaction was filtered and concentrated to afford the title compound (1.15 g, 3.24 mmol, 91% yield) as a white solid. MS (ESI): m/z=300.3 [M-C₄H₈+H]⁺

### Example B.529

### 6-[[4-(trifluoromethylsulfonyl)pyrazol-1-yl]methyl]-2-azaspiro[3.3]heptane 4-methylbenzenesulfonic Acid

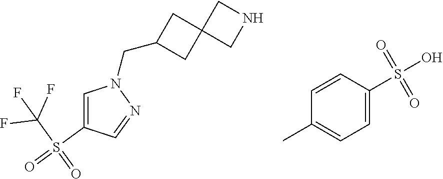 [SMILES: O=S(=O)(c1cnn(CC2CC3(CNC3)C2)c1)C(F)(F)F ; Cc1ccc(S(=O)(=O)O)cc1]

A mixture of tert-butyl 6-[[4-(trifluoromethylsulfonyl)pyrazol-1-yl]methyl]-2-azaspiro[3.3]heptane-2-carboxylate (630 mg, 1.54 mmol) and p-toluenesulfonic acid (317.97 mg, 1.85 mmol, 1.2 eq) in EtOAc (20 mL) was stirred at 80° C. for 12 h under N₂ balloon. The reaction was concentrated and the residue was dissolved in H₂O (30 mL). The solution was lyophilized to afford the title compound (738 mg, 1.53 mmol, 97% yield) as a light yellow solid. MS (ESI): m/z=310.1 [M+H]⁺

### Step a) Tert-Butyl 6-[[4-(trifluoromethylsulfanyl)pyrazol-1 yl]methylene]-2-azaspiro[3.3]heptane-2-carboxylate

To a solution of tert-butyl 6-[(4-iodopyrazol-1-yl)methylene]-2-azaspiro[3.3]heptane-2-carboxylate (B.528, Step a) (30.0 mg, 0.07 mmol) in ACN (3 mL) was added silver(I) Trifluoromethanethiolate (46.9 mg, 0.22 mmol), CuI (28.5 mg, 0.15 mmol) and 2-pyridin-2-ylpyridine (23.4 mg, 0.15 mmol) in a sealed tube at 90° C. for 12 h. The reaction was concentrated. The residue was purified by prep-HPLC (water (FA)-ACN] 52%-82%); to afford the title compound (13.0 mg, 0.03 mmol, 46% yield) as a yellow solid. MS (ESI): m/z=320.1 [M-C₄H₈+H]⁺

### Step b) Tert-Butyl 6-[[4-(trifluoromethylsulfanyl)pyrazol-1 yl]methyl]-2-azaspiro[3.3]heptane-2-carboxylate

To a solution of tert-butyl 6-[[4-(trifluoromethylsulfanyl)pyrazol-1-yl]methylene]-2-azaspiro[3.3]heptane-2-carboxylate (2.8 g, 7.46 mmol) in EtOAc (50 mL) was added Pd/C (1.0 g, 0.75 mmol) at 25° C. and stirred for 1 h under H₂ balloon (760 mm Hg). The reaction was filtered and concentrated to afford the title compound (2.8 g, 7.42 mmol, 99% yield) as a white solid. MS (ESI): m/z=322.2 [M-C₄H₈+H]⁺

### Step c) Tert-Butyl 6-[[4-(trifluoromethylsulfonyl)pyrazol-1-yl]methyl]-2-azaspiro[3.3]heptane-2-carboxylate

To a solution of tert-butyl 6-[[4-(trifluoromethylsulfanyl)pyrazol-1-yl]methyl]-2-azaspiro[3.3]heptane-2-carboxylate (1.0 g, 2.65 mmol) in 1,2-dichloroethane (4 mL), ACN (4 mL) and water (8 mL) was added sodium periodate (1.13 g, 5.3 mmol) and ruthenium(III) chloride hydrate (59.7 mg, 0.26 mmol) at 0° C., then the mixture was stirred at 25° C. for 12 h. The reaction was concentrated. The residue was purified by prep-HPLC (water(FA)-ACN 50%-80%) to afford the title compound (630 mg, 1.54 mmol, 58% yield) as a grey solid. MS (ESI): m/z=354.3 [M-C₄H₈+H]⁺

### Example B.530

### [1-(2-azaspiro[3.3]heptan-6-ylmethyl)pyrazol-4-yl]-imino-oxo-(trifluoromethyl)-λ⁶-sulfane; 4-methylbenzenesulfonic Acid

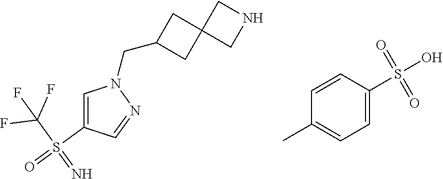 [SMILES: N=S(=O)(c1cnn(CC2CC3(CNC3)C2)c1)C(F)(F)F ; Cc1ccc(S(=O)(=O)O)cc1]

A mixture of tert-butyl 6-[[4-(trifluoromethylsulfonimidoyl)pyrazol-1-yl]methyl]-2-azaspiro[3.3]heptane-2-carboxylate (650 mg, 1.59 mmol) and p-toluenesulfonic acid (329 mg, 1.91 mmol) in EtOAc (10 mL) was stirred at 80° C. for 12 h under N₂ balloon. The reaction was concentrated and the residue was dissolved in H₂O (30 mL). The solution was lyophilized to the title compound (623 mg, 1.3 mmol, 77% yield) as a light yellow oil. MS (ESI): m/z=309.1 [M+H]⁺

### Step a) Tert-Butyl 6-[[4-(trifluoromethylsulfonimidoyl)pyrazol-1 yl]methyl]-2-azaspiro[3.3]heptane-2-carboxylate

A mixture of tert-butyl 6-[[4-(trifluoromethylsulfanyl)pyrazol-1-yl]methyl]-2-azaspiro[3.3]heptane-2-carboxylate (B.529, Step b) (1.8 g, 4.77 mmol), PhI(OAc)₂ (6.14 g, 19.1 mmol) and NH₂COONH₄ (1.49 g, 19.1 mmol) in TFE was stirred at 30° C. for 12 h. The reaction was concentrated. The residue was purified by prep-HPLC (water (FA)-ACN, 60%-80%) to afford the title compound (650 mg, 1.59 mmol, 33% yield) as a grey solid. MS (ESI): m/z=353.1 [M-C₄H₈+H]⁺

### Example B.531

### 6-[difluoro-[5-(trifluoromethyl)pyrazin-2-yl]methyl]-2-azaspiro[3.3]heptane; 4-methylbenzenesulfonic Acid

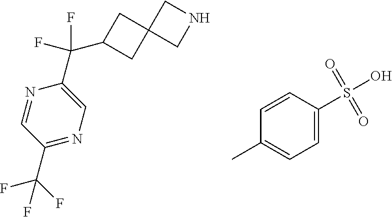 [SMILES: FC(F)(F)c1cnc(C(F)(F)C2CC3(CNC3)C2)cn1 ; Cc1ccc(S(=O)(=O)O)cc1]

To a solution of 6-[difluoro-[6-(trifluoromethyl)-3-pyridyl]methyl]-2-azaspiro[3.3]heptane-2-carboxylic acid tert-butyl ester (77 mg, 196 μmol) in isopropyl acetate (3 mL) was added p-toluenesulfonic acid monohydrate (82.1 mg, 432 μmol). The mixture was stirred at 80° C. for 3 h. The reaction mixture was concentrated in vacuo to afford 6-[difluoro-[6-(trifluoromethyl)-3-pyridyl]methyl]-2-azaspiro[3.3]heptane 1:2 4-methylbenzenesulfonic acid (163 mg, 99%) as a colorless oil. MS (ESI): m/z=293.0 [M+H]⁺

### Step a) Tert-Butyl 6-[5-(trifluoromethyl)pyrazine-2-carbonyl]-2-azaspiro[3.3]-2-carboxylate

To a solution of tert-butyl 6-[[5-(trifluoromethyl)pyrazin-2-yl]methyl]-2-azaspiro[3.3]heptane-2-carboxylate (B.4, Step c) (5.3 g, 14.8 mmol) in 1,4-Dioxane (2 mL) was added SeO₂ (16.5 g, 148 mmol). The reaction mixture was stirred at 105° C. for 4 h. The reaction was concentrated. The residue was purified by prep-HPLC (water(FA)-ACN, 50%-80%) to afford the crude title compound (550 mg, 1.48 mmol, 10% yield) as a yellow solid. MS (ESI): m/z=316.1 [M-C₄H₈+H]⁺; ¹H NMR (400 MHz, CHLOROFORM-d) δ=9.25 (s, 1H), 8.91 (s, 1H), 3.96 (s, 2H), 3.80 (s, 2H), 2.66-2.49 (m, 1H), 2.45 (d, J=8.5 Hz, 4H), 1.36 (s, 9H)

### Step b) 6-[difluoro-[6-(trifluoromethyl)-3-pyridyl]methyl]-2-azaspiro[3.3]heptane-2-carboxylic Acid Tert-Butyl Ester

To a solution of tert-butyl 6-[6-(trifluoromethyl)pyridine-3-carbonyl]-2-azaspiro[3.3]heptane-2-carboxylate (750 mg, 2.03 mmol) in DCM (5 mL) was added DAST (5.0 mL, 37.8 mmol) dropwise at 0° C. under N₂ atmosphere. The reaction mixture was stirred at 25° C. for 12 h. The reaction mixture was poured into saturated NaHCO₃ aqueous solution (200 mL) dropwise and then extracted with DCM (50 mL×2). The combined organic layers were dried over Na₂SO₄, filtered and concentrated. The residue was purified by prep-HPLC (water(FA)-ACN, 58%-88%); to afford the title compound (510 mg, 1.3 mmol, 64% yield) as a light yellow solid. MS (ESI): m/z=337.0 [M-C₄H₈+H]⁺

In analogy to Example B.531, the following building blocks were generated using the relevant commercial building blocks.

| | | | | MS, |
|---|---|---|---|---|
| | | | | ESI: |
| Ex. | Structure | Systematic Name | Building Blocks | m/z |
| B.532 | 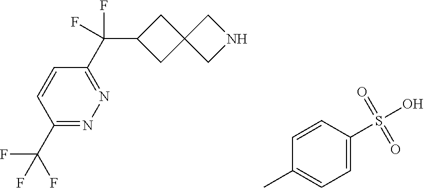 [SMILES: FC(F)(F)c1ccc(C(F)(F)C2CC3(CNC3)C2)nn1 ; Cc1ccc(S(=O)(=O)O)cc1] | 6-[difluoro-[6- (trifluoromethyl) pyridazin- 3-yl\|methyl]-2- azaspiro[3.3]heptane; 4- methylbenzenesulfonic acid | tert-butyl 6-[[6- (trifluoromethyl) pyridazin-3- yl]methyl]-2- azaspiro[3.3] heptane- 2-carboxylate (B.11, Step c) | 294.0 [M − TsOH + H]⁺ |

### Example B.533

### 6-[difluoro-[6-(trifluoromethyl)-3-pyridyl]methyl]-2-azaspiro[3.3]heptane; 4-methylbenzenesulfonic Acid

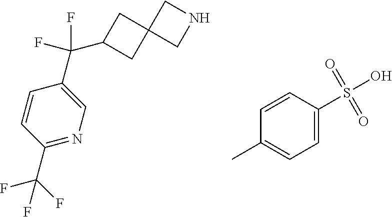 [SMILES: FC(F)(F)c1ccc(C(F)(F)C2CC3(CNC3)C2)cn1 ; Cc1ccc(S(=O)(=O)O)cc1]

To a solution of 6-[difluoro-[6-(trifluoromethyl)-3-pyridyl]methyl]-2-azaspiro[3.3]heptane-2-carboxylic acid tert-butyl ester (77 mg, 196 μmol) in isopropyl acetate (3 mL) was added p-toluenesulfonic acid monohydrate (82.1 mg, 432 μmol). The mixture was stirred at 80° C. for 3 h. The reaction mixture was concentrated in vacuo to afford the title compound (163 mg, 99%) as colorless oil. MS (ESI): m/z=293.0 [M+H]⁺

### Step a) Tert-Butyl 6-[6-(trifluoromethyl)pyridine-3-carbonyl]-2-azaspiro[3.3]heptane-2-carboxylate

To a solution of tert-butyl 6-[methoxy(methyl)carbamoyl]-2-azaspiro[3.3]heptane-2-carboxylate (CAS: 2428601-18-5) (4.3 g, 15.1 mmol), and 5-bromo-2-(trifluoromethyl)pyridine (6.83 g, 30.2 mmol) in THE (100 mL) was added n-BuLi (18.2 mL, 45.4 mmol) at −78° C. for 1 h. Then the reaction mixture was stirred at 30° C. for 12 h. The reaction mixture was poured into saturated NH₄Cl aqueous solution (500 mL) and then extracted with ethyl acetate (60 mL×3). The organic layer was washed with brine.

The residue was purified by flash chromatography on silica gel eluting with PE/EtOAc (2:1) to afford the title compound (1400 mg, 3.78 mmol, 25% yield) as a brown solid. ¹H NMR (400 MHz, METHANOL-d4) δ=9.17 (s, 1H), 8.49 (dd, J=1.5, 8.3 Hz, 1H), 7.95 (d, J=8.3 Hz, 1H), 4.11-3.99 (m, 3H), 3.84 (s, 2H), 2.65-2.48 (m, 4H), 1.43 (s, 9H).

Note that regioisomer was generated concurrently: tert-butyl 6-[2-(trifluoromethyl)pyridine-4-carbonyl]-2-azaspiro[3.3]heptane-2-carboxylate (1100 mg, 2.97 mmol, 20% yield) as a brown oil. ¹H NMR (400 MHz, METHANOL-d4) δ=8.92 (d, J=5.0 Hz, 1H), 8.15 (s, 1H), 8.04 (dd, J=1.1, 4.9 Hz, 1H), 4.03 (s, 3H), 3.83 (s, 2H), 2.55 (br dd, J=8.3, 12.1 Hz, 4H), 1.43 (s, 9H)

### Step b) 6-[difluoro-[6-(trifluoromethyl)-3-pyridyl]methyl]-2-azaspiro[3.3]heptane-2-carboxylic Acid Tert-Butyl Ester

To a solution of tert-butyl 6-[6-(trifluoromethyl)pyridine-3-carbonyl]-2-azaspiro[3.3]heptane-2-carboxylate (750 mg, 2.03 mmol) in DCM (5 mL) was added DAST (5.0 mL, 37.8 mmol) dropwise at 0° C. under N₂ atmosphere. The reaction mixture was stirred at 25° C. for 12 h. The reaction mixture was added into saturated NaHCO₃ aqueous solution (200 mL) dropwise and then extracted with DCM (50 mL×2). The combined organic layers were dried over Na₂SO₄, filtered and concentrated. The residue was purified by prep-HPLC (water(FA)-ACN, 58%-88%); to afford the title compound (510 mg, 1.3 mmol, 64% yield) as a light yellow solid. MS (ESI): m/z=337.0 [M-C₄H₈+H]⁺; ¹H NMR (400 MHz, METHANOL-d4) δ=8.87 (s, 1H), 8.26-8.13 (m, 1H), 7.94 (d, J=8.3 Hz, 1H), 4.03-3.83 (m, 4H), 3.18-2.96 (m, 1H), 2.47-2.27 (m, 4H), 1.44 (s, 9H)

### Example B.534

### 6-[fluoro-[6-(trifluoromethyl)-3-pyridyl]methyl]-2-azaspiro[3.3]heptane; 4-methylbenzenesulfonic Acid

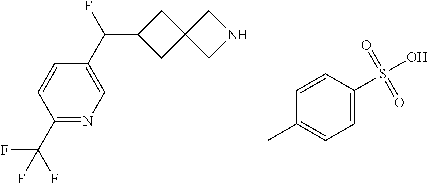 [SMILES: FC(c1ccc(C(F)(F)F)nc1)C1CC2(CNC2)C1 ; Cc1ccc(S(=O)(=O)O)cc1]

To an solution of 6-[fluoro-[6-(trifluoromethyl)-3-pyridyl]methyl]-2-azaspiro[3.3]heptane-2-carboxylic acid tert-butyl ester (42 mg, 112 μmol) in ethyl acetate (3.2 mL) was added p-toluenesulfonic acid (38.6 mg, 224 μmol) and the mixture was heated at reflux for 1 h. The clear, colorless solution was allowed to cool down to RT, and was concentrated in a rotary evaporator and dried on HV to yield the title compound (90 mg). MS (ESI): m/z=275.1 [M-TsOH+H]⁺

### Step a) 6-[hydroxy-[6-(trifluoromethyl)-3 pyridyl]methyl]-2-azaspiro[3.3]heptane-2-carboxylic Acid Tert-Butyl Ester

The 1.3 M isopropylmagnesium chloride lithium chloride complex solution, 1.3 M in THF (1.7 mL, 2.21 mmol) was cooled to 0° C. A solution of 5-bromo-2-(trifluoromethyl)pyridine (500 mg, 2.21 mmol) in tetrahydrofuran (3 mL) was added dropwise. The mixture was stirred for 1 h at RT, then cooled again to 0° C. and a solution of 6-formyl-2-azaspiro[3.3]heptane-2-carboxylic acid tert-butyl ester (CAS: 1440960-67-7) (548 mg, 2.43 mmol) in THF was added. After the addition was complete, the mixture was warmed to RT and stirred for 1.5 h. The reaction was quenched at 0° C. by addition of ammonium chloride (aq.) solution, and the mixture was then diluted with water and extracted with EtOAc (×2). The organic phases were dried over Na₂SO₄, filtered and concentrated in vacuo. The residue was purified by silica column chromatography (heptane/EtOAc 0-60%) to yield the title compound (400 mg, 46%) as a colorless foam. MS (ESI): m/z=417.2 [M−C₄H₈+H]⁺

### Step b) 6-[fluoro-[6-(trifluoromethyl)-3 pyridyl]methyl]-2-azaspiro[3.3]heptane-2-carboxylic Acid Tert-Butyl Ester

6-[hydroxy-[6-(trifluoromethyl)-3-pyridyl]methyl]-2-azaspiro[3.3]heptane-2-carboxylic acid tert-butyl ester (150 mg, 403 μmol) was dessolved in dichloromethane (4.62 mL) and cooled to −20° C. Then 1.3 M 4-morpholinylsulfur trifluoride (449 mg, 310 μL, 403 μmol) was added, and the mixture stirred for 1 h while it warmed from −20° C. and a further 1 h at 0° C. The reaction was quenched with ice, diluted with 2M KHCO₃ (aq.) solution, and extracted with 2×DCM. The organic phases were dried over sodium sulfate, filtered and concentrated in vacuo. The residue was purified by silica column chromatography (heptane/EtOAc, 0%-80%) to yield the title compound (42 mg, 26% yield) as a colorless oil. MS (ESI): m/z=319.0 [M−C₄H₈+H]⁺

### Example B.535

### 2-(2-azaspiro[3.5]nonan-7-ylmethyl)-5-(trifluoromethyl)-1,3,4-oxadiazole; 4-methylbenzenesulfonic Acid

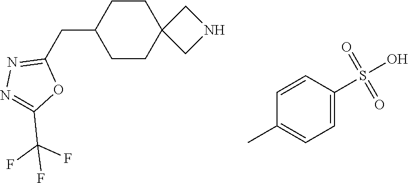 [SMILES: FC(F)(F)c1nnc(CC2CCC3(CC2)CNC3)o1 ; Cc1ccc(S(=O)(=O)O)cc1]

To a solution of 7-[[5-(trifluoromethyl)-1,3,4-oxadiazol-2-yl]methyl]-2-azaspiro[3.5]nonane-2-carboxylic acid tert-butyl ester (46.8 mg, 0.125 mmol) in dichloromethane (0.5 mL) was added TFA (142 mg, 96.1 μL, 1.25 mmol) and the reaction mixture was stirred at RT for 5 h. Volatiles were removed in vacuo to yield 95 mg of the crude title compound which was used without further purification, purity roughly 50% with major contaminant excess of TFA. MS (ESI): m/z=276.3 [M-TsOH+H]⁺

### Step a) Tert-Butyl 7-(2-ethoxy-2-oxo-ethyl)-2-azaspiro[3.5]nonane-2-carboxylate

To a solution of triethyl phosphonoacetate (4500 mg, 20.1 mmol) in THE (8 mL), NaH (963 mg, 24.1 mmol, 1.2 eq) was added slowly at 0° C. and stirred for 30 mins, then tert-butyl 7-oxo-2-azaspiro[3.5]nonane-2-carboxylate (CAS: 1363381-22-9) (5284 mg, 22.1 mmol) in THE (200 mL) was added and stirred at 0° C. for 1.5 h. The reaction solution was quenched with H₂O, extracted with EtOAc, the organic layer was purified with silica chromatography column (100% PE to PE:EA=10:1) and evaporated to give the title compound (5500 mg, 17.8 mmol, 89% yield) as colorless oil. MS (ESI): m/z=254.2 [M−C₄H₈+H]⁺

### Step b) Tert-Butyl 7-(2-hydrazino-2-oxo-ethyl)-2-azaspiro[3.5]nonane-2-carboxylate

To a solution of tert-butyl 7-(2-ethoxy-2-oxo-ethyl)-2-azaspiro[3.5]nonane-2-carboxylate (1500 mg, 4.82 mmol) and N₂H₄·H₂O (369 mg, 7.22 mmol) in ethanol (15 mL) was stirred at 80° C. for 12 h. A further addition of N₂H₄·H₂O (425 mg, 7.22 mmol) was made, and the mixture stirred at 80° C. for 12 h. The residue was poured into water. The aqueous phase was extracted with ethyl acetate (30 mL×3). The combined organic phase was washed with brine (30 mL×3), dried with anhydrous Na₂SO₄, filtered and concentrated in vacuum to give the title compound (700 mg, 2.35 mmol, 49% yield) as white solid. MS (ESI): m/z=242.2 [M−C₄H₈+H]⁺

### Step b) Tert-Butyl 7-[2-oxo-2-[2-(2,2,2-trifluoroacetyl)hydrazino]ethyl]-2-azaspiro[3.5]nonane-2-carboxylate

To a solution of tert-butyl 7-(2-hydrazino-2-oxo-ethyl)-2-azaspiro[3.5]nonane-2-carboxylate (1.2 g, 4.04 mmol) in THE (3.12 mL), trifluoroacetic anhydride (1.14 mL, 8.07 mmol) was added and stirred at 25° C. for 2 h. Saturated aqueous NaHCO₃ solution was added, The aqueous phase was extracted with ethyl acetate (30 mL×3). The combined organic phase was washed with brine (30 mL×3), dried with anhydrous Na₂SO₄, filtered and concentrated in vacuum. The crude product was purified by reversed-phase HPLC (0.1% FA condition) to give the title compound (330 mg, 0.84 mmol, 21% yield); MS (ESI): m/z=338.1 [M+H]⁺, along with impurity 2,2,2-trifluoro-N′-[2-[2-(2,2,2-trifluoroacetyl)-2-azaspiro[3.5]nonan-7-yl]acetyl]acetohydrazide (640 mg, 1.64 mmol, 41% yield).

### Step c) 7-[[5-(trifluoromethyl)-1,3,4-oxadiazol-2 yl]methyl]-2-azaspiro[3.5]nonane-2-carboxylic Acid Tert-Butyl Ester

A solution of PPh₃ (386 mg, 1.47 mmol) in THE (3 mL) was purged with N₂ three times, then iodine (374 mg, 1.47 mmol), TEA (373 mg, 3.69 mmol) and tert-butyl 7-[2-oxo-2-[2-(2,2,2-trifluoroacetyl)hydrazino]ethyl]-2-azaspiro[3.5]nonane-2-carboxylate (290 mg, 0.74 mmol) was added and stirred at 25° C. for 12 h. The reaction mixture was concentrated under reduced pressure to give a residue. The residue was purified by prep-HPLC (water(FA)-ACN) and lyophized to give the title compound (70 mg, 0.19 mmol, 25 yield) as yellow solid; MS (ESI): m/z=319.9 [M+H]⁺

### Example B.539

### [4-(azetidin 3-yl)phenyl]-imino-oxo-(trifluoromethyl)-λ⁶-sulfane; 4-methylbenzenesulfonic Acid

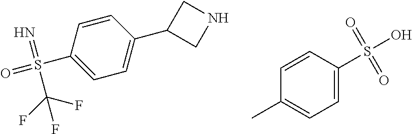 [SMILES: N=S(=O)(c1ccc(C2CNC2)cc1)C(F)(F)F ; Cc1ccc(S(=O)(=O)O)cc1]

A mixture of tert-butyl 3-[4-(trifluoromethylsulfonimidoyl)phenyl]azetidine-1-carboxylate (620 mg, 1.7 mmol) and p-toluenesulfonic acid (381 mg, 2.21 mmol) in EtOAc (6 mL) was stirred at 80° C. for 12 h. To the mixture was added p-toluenesulfonic acid (29.3 mg, 0.17 mmol) and stirred for another 12 h at 80° C. The reaction was concentrated under vacuum to give a residue. To the residue was added 35 mL water and lyophilized to give the title compound (740 mg, 1.7 mmol, 99% yield) as white solid. MS (ESI): m/z=265.2 [M-TsOH+H]⁺

### Step a) Tert-Butyl 3-[4-(trifluoromethylsulfanyl)phenyl]azetidine-1-carboxylate

To a solution of tert-butyl 3-bromoazetidine-1-carboxylate (CAS: 1064194-10-0) (1194 mg, 5.06 mmol), 4-(trifluoromethylthio)bromobenzene (CAS: 333-47-1) (1000 mg, 3.89 mmol), Ir[dF(CF₃)ppy]₂(dtbpy)(PF₆) (43.61 mg, 0.04 mmol), NiCl₂·dtbbpy (7.74 mg, 0.02 mmol), TTMSS (967 mg, 3.89 mmol), Na₂CO₃ (825 mg, 7.78 mmol) in DME (20 mL). The vial was sealed and placed under nitrogen was added. The reaction was stirred and irradiated with a 34 W blue LED lamp (7 cm away), with cooling fan to keep the reaction temperature at 25° C. for 14 h. The residue was purified by reversed-phase HPLC (0.1% FA condition) to give the title compound (590 mg, 1.77 mmol, 46% yield) as light brown oil. MS (ESI): m/z=278.1 [M-C₄H₈+H]⁺

### Step b) Tert-Butyl 3-[4-(trifluoromethylsulfonimidoyl)phenyl]azetidine-1-carboxylate

To a solution of tert-butyl 3-[4-(trifluoromethylsulfanyl)phenyl]azetidine-1-carboxylate (590 mg, 1.77 mmol) in trifluoroethanol (6.0 mL) was added PhI(OAc)₂ (2394 mg, 7.43 mmol), NH₂COONH₄ (414 mg, 5.31 mmol) at 25° C. The mixture was stirred at 60° C. for 3 h. Two equivalent reactions were combined for workup. The combined reaction mixture was concentrated in vacuo. The residue was purified by flash silica gel chromatography (eluent of 0-30% Ethyl acetate/Petroleum ether) to give the title compound (600 mg, 1.65 mmol, 93% yield) as yellow oil. MS (ESI): m/z=309.1 [M-C₄H₈+H]⁺

### Example B.540

### 3-[4(1-methylsulfonylcyclopropyl)phenyl]azetidine; 4-methylbenzenesulfonic Acid

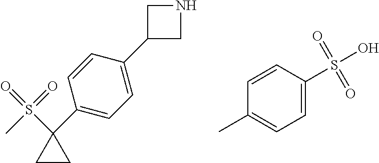 [SMILES: CS(=O)(=O)C1(c2ccc(C3CNC3)cc2)CC1 ; Cc1ccc(S(=O)(=O)O)cc1]

A solution of tert-butyl 3-[4-(1-methylsulfonylcyclopropyl)phenyl]azetidine-1-carboxylate (300 mg, 0.85 mmol) and p-toluenesulfonic acid monohydrate (243.55 mg, 1.28 mmol, 1.5 eq) in EtOAc (15 mL)/TBME (15 mL) was stirred at 25° C. for 18 h. The solvent was evaporated and obtained residue was stirred with TBME (150 mL) for 18 h. The obtained precipitate was filtered, washed with TBME and dried in vacuum to give the title compound (285 mg, 0.67 mmol, 72% yield) as light yellow solid. MS (ESI): m/z=252.2 [M+H]⁺

### Step a) Tert-Butyl 3-[4-(1-methylsulfonylvinyl)phenyl]azetidine-1-carboxylate

A mixture of tert-butyl 3-[4-(4,4,5,5-tetramethyl-1,3,2-dioxaborolan-2-yl)phenyl]azetidine-1-carboxylate (CAS: 1613259-77-0) (2900 mg, 8.07 mmol), 1-bromo-1-methylsulfonyl-ethylene (CAS: 35495-31-9) (1718 mg, 9.28 mmol), Pd(dppf)Cl₂·CH₂Cl₂ (989 mg, 1.21 mmol), potassium carbonate (2231 mg, 16.1 mmol) in THF (50 mL) and Water (7 mL) was heated at 70° C. in a sealed vial under Argon atmosphere for 18 h. Then RM was cooled to RT, filtered, quenched with water and extracted with EtOAc (3×50 mL). The combined organic layer was washed with brine, dried over Na₂SO₄, filtered and concentrated under reduced pressure. The residue was purified by column chromatography (hexane/MTBE 0-70%), to give the title compound (870 mg, 2.58 mmol, 30% yield) as light red semisolid. MS (ESI): m/z=238.2 [M+H]⁺

### Step b) Tert-Butyl 3-[4-(1-methylsulfonylcyclopropyl)phenyl]azetidine-1-carboxylate

To a stirred solution of diphenyl(methyl)sulfonium tetrafluoroborate (1114 mg, 3.87 mmol) and tert-butyl 3-[4-(1-methylsulfonylvinyl)phenyl]azetidine-1-carboxylate (870 mg, 2.58 mmol) in anhydrous THF (25 mL), sodium bis(trimethylsilyl)amide (2.0 mL, 4.64 mmol) (40% in THF) was added at 0° C. under Ar. The reaction mixture was stirred at 0° C. for 10 min and then at 25° C. for 10 h. The reaction mixture was quenched with aqueous saturated ammonium chloride solution (15 mL), diluted with water (50 mL) and the organics were extracted with ethyl acetate (2×50 mL). The organic layer was washed with brine, dried over sodium sulfate, filtered and the solvent was evaporated in vacuo. The crude product was purified by column chromatography (hexane/TBME 0-80%) to give the title compound (300 mg, 0.85 mmol, 31% yield) as light yellow solid. MS (ESI): m/z=252.2 [M-Boc+H]⁺

### Example B.542 and B.543

### 6-[(1R)-1-[4-methyl-5-(trifluoromethyl)-2-pyridyl]ethyl]-2-azaspiro[3.3]heptane; 4-methylbenzenesulfonic acid and 6-[(1S)-1-[4-methyl-5-(trifluoromethyl)-2-pyridyl]ethyl]-2-azaspiro[3.3]heptane; 4-methylbenzenesulfonic Acid

### (Arbitrary Assignment of Enantiomers)

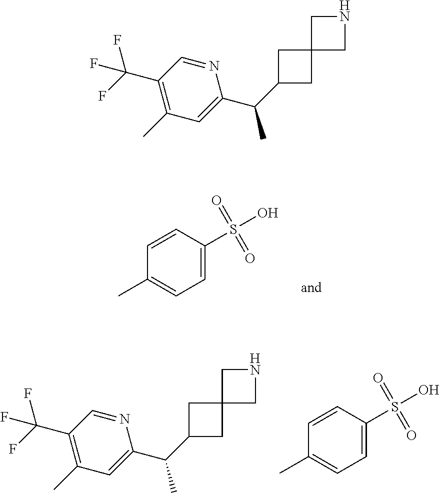 [SMILES: Cc1cc([C@H](C)C2CC3(CNC3)C2)ncc1C(F)(F)F ; Cc1ccc(S(=O)(=O)O)cc1 ; Cc1cc([C@@H](C)C2CC3(CNC3)C2)ncc1C(F)(F)F ; Cc1ccc(S(=O)(=O)O)cc1]

The solution of tert-butyl 6-[(I R)-1-[4-methyl-5-(trifluoromethyl)-2-pyridyl]ethyl]-2-azaspiro[3.3]heptane-2-carboxylate (250 mg, 0.65 mmol) and p-toluenesulfonic acid monohydrate (309 mg, 1.63 mmol) in EtOAc (10 mL) was stirred at 60° C. for 3 h. After cooling to RT, the obtained precipitate was filtered, washed with EtOAc and dried in vacuum to give the title compound B.542 (237 mg, 0.38 mmol, 55% yield) as white solid. MS (ESI): m/z=285.2 [M+H]⁺. Deprotection of the other enantiomer carried out in analogy to yield title compound B.543 (241 mg, 0.38 mmol, 58% yield) as white solid. MS (ESI): m/z=285.2 [M+H]⁺

### Step a) Tert-Butyl 6-[1-(4,4,5,5-tetramethyl-1,3,2-dioxaborolan-2-yl)ethylidene]-2-azaspiro[3.3]heptane-2-carboxylate

To a stirred −30° C. solution of 2,2,6,6-tetramethylpiperidine (4.31 mL, 25.6 mmol) in THE (75 mL) under Ar, butyllithium solution (2.5 M in hexane) (10.2 mL, 25.6 mmol) was added. The solution was stirred at −30° C. for 30 min and cooled to −78° C. The solution of 4,4,5,5-tetramethyl-2-[1-(4,4,5,5-tetramethyl-1,3,2-dioxaborolan-2-yl)ethyl]-1,3,2-dioxaborolane (CAS: 1227056-25-8) (6.61 g, 23.4 mmol) in 10 mL of dry THE was added dropwise and reaction mixture was stirred at −78° C. for 30 min. The solution of tert-butyl 6-oxo-2-azaspiro[3.3]heptane-2-carboxylate (4500 mg, 21.3 mmol) in 15 mL of dry THE was added dropwise at −78° C. and reaction mixture was allowed to warm and stirred overnight at room temperature. The reaction was quenched with water; the mixture was vacuum-concentrated, and the residue was separated between water and DCM. The organic layer was washed with citric acid, water, brine, dried over sodium sulfate and concentrated to give residue which was purified by column chromatography (1:4 TBME/hexane) to give the title compound (3.1 g, 8.88 mmol, 41% yield) as a white solid. MS (ESI): m/z=294.2 [M-C₄H₈+H]⁺

### Step b) Tert-Butyl 6-[1-[4-methyl-5-(trifluoromethyl)-2 pyridyl]ethylidene]-2-azaspiro[3.3]heptane-2-carboxylate

To a stirred suspension of 2-chloro-4-methyl-5-(trifluoromethyl)pyridine (1.36 g, 6.93 mmol), tert-butyl 6-[1-(4,4,5,5-tetramethyl-1,3,2-dioxaborolan-2-yl)ethylidene]-2-azaspiro[3.3]heptane-2-carboxylate (2.2 g, 6.3 mmol) and cesium carbonate (4.1 g, 12.6 mmol) in dry 1,4-Dioxane (110 mL), flushed with Argon for 3 minutes, 1,1′-bis(diphenylphosphino)ferrocene-palladium(II)dichloride dichloromethane complex (771 mg, 0.94 mmol) was added. The mixture was stirred at 80° C. for 48 h under Argon atmosphere. After cooling to RT, the reaction mixture was filtered and filtrate was concentrated. The obtained residue was partitioned between EtOAc and brine. The organic layer was dried over Na₂SO₄ and concentrated to give crude product, which was purified by column chromatography (hexane/MTBE 10-23%) to obtain the title compound (700 mg, 1.83 mmol, 28% yield) as light yellow solid. MS (ESI): m/z=327.2 [M-C₄H₈+H]⁺

### Step c) Tert-Butyl 6-[1-[4-methyl-5-(trifluoromethyl)-1 pyridyl]ethyl]-2-azaspiro[3.3]heptane-2-carboxylate

The stirred mixture of tert-butyl 6-[1-[4-methyl-5-(trifluoromethyl)-2-pyridyl]ethylidene]-2-azaspiro[3.3]heptane-2-carboxylate (700 mg, 1.83 mmol) and Pd/C (5%) (200 mg) in EtOAc (50 mL) was hydrogenated in autoclave at 15200 mmHg (20 bar) for 24 h at 25° C. The reaction mixture was filtered and concentrated in vacuum to give the title compound (680 mg, 1.77 mmol, 92% yield) as orange gum; MS (ESI): m/z=329.0 [M-C₄H₈+H]⁺. A mixture of enantiomers tert-butyl 6-[1-[4-methyl-5-(trifluoromethyl)-2-pyridyl]ethyl]-2-azaspiro[3.3]heptane-2-carboxylate (680 mg, 1.77 mmol) was separated by chiral chromatography to give tert-butyl 6-[(1S)-1-[4-methyl-5-(trifluoromethyl)-2-pyridyl]ethyl]-2-azaspiro[3.3]heptane-2-carboxylate (270 mg, 0.7 mmol, 40% yield) (or enantiomer) RetTime (min): 11.2 and tert-butyl 6-[(1R)-1-[4-methyl-5-(trifluoromethyl)-2-pyridyl]ethyl]-2-azaspiro[3.3]heptane-2-carboxylate (250 mg, 0.65 mmol, 37% yield) (or enantiomer) RetTime(min): 13.9.

### Example B.548

### [4-(azetidin-3-yl)phenyl]-cyclopropyl-imino-oxo-4′-sulfane; 4-methylbenzenesulfonic Acid

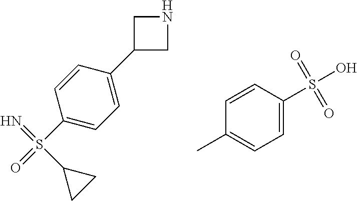 [SMILES: N=S(=O)(c1ccc(C2CNC2)cc1)C1CC1 ; Cc1ccc(S(=O)(=O)O)cc1]

A solution of p-toluenesulfonic acid (709 mg, 4.12 mmol) and tert-butyl 3-[4-(cyclopropylsulfonimidoyl)phenyl]azetidine-1-carboxylate (630 mg, 1.87 mmol) in EtOAc (8 mL) was stirred at 80° C. for 12 h. The mixture was concentrated under vacuum to give a residue. Deionized water was added to the residue and the crude was lyophilized to give the title compound (917 mg, 1.58 mmol, 84% yield) as a white solid. MS (ESI): m/z=237.1 [M−2TsOH+H]⁺

### Step a) 1-promo-4-cyclopropylsulfanyl-benzene

To a solution of 4-bromothiophenol (5.0 g, 26.5 mmol) in DMSO (125 mL) was added potassium tert-butoxide (6.53 g, 58.2 mmol), cyclopropyl bromide (19196 mg, 159 mmol) at 20° C. The mixture was stirred at 80° C. for 12 h under N₂. A further addition of cyclopropyl bromide (19196 mg, 159 mmol) was made, with stirring at 80° C. for 12 h. The reaction mixture was diluted with 200 mL water, extracted with 100×3 mL EtOAc. The combined organic layers were washed with 100 mL brine, dried over Na₂SO₄ and filtered. The filtrate was concentrated under reduced pressure to give a residue. The residue was purified by column chromatography (Petroleum ether/Ethyl acetate=1:0) and concentrated under reduced pressure to give the title compound (3700 mg, 16.2 mmol, 61% yield) as a yellow oil. ¹H NMR (400 MHz, CHLOROFORM-d) δ ppm 7.35-7.45 (m, 2H) 7.19-7.26 (m, 2H) 2.11-2.22 (m, 1H) 1.03-1.16 (m, 2H) 0.64-0.75 (m, 2H)

### Step b) Tert-Butyl 3-(4-cyclopropylsulfanylphenyl)azetidine-1-carboxylate

To a solution of tert-butyl 3-bromoazetidine-1-carboxylate (2380 mg, 10.1 mmol), 1-bromo-4-cyclopropylsulfanyl-benzene (1540 mg, 6.72 mmol), Ir[dF(CF₃)ppy]₂(dtbpy)(PF₆) (75.3 mg, 0.07 mmol), NiCl₂·dtbbpy (13.4 mg, 0.03 mmol), TTMSS (1671 mg, 6.72 mmol), Na₂CO₃ (1425 mg, 13.4 mmol) in DME (40 mL). The vial was sealed and placed under nitrogen, and the reaction was stirred and irradiated with a 34 W blue LED lamp (7 cm away), with cooling fan to keep the reaction temperature at 20° C. for 14 h. The reaction was filtered and the filtrate was concentrated under reduced pressure to give a residue. The residue was purified by rev, column chromatography (Petroleum ether/Ethyl acetate=10:1). The purified solution was concentrated under reduced pressure to give a tert-butyl 3-(4-cyclopropylsulfanylphenyl)azetidine-1-carboxylate (2600 mg, 8.51 mmol, 89% yield) as a yellow oil. MS (ESI): m/z=250.2 [M-C₄H₈+H]+

### Step c) Tert-Butyl 3-[4-(cyclopropylsulfonimidoyl)phenyl]azetidine-1-carboxylate

To a solution of tert-butyl 3-(4-cyclopropylsulfanylphenyl)azetidine-1-carboxylate (2600 mg, 8.51 mmol) in trifluoroethanol (55.0 mL, 52.0 mmol) was added PhI(OAc)₂ (11516 mg, 35.8 mmol), NH₂COONH₄ (1994 mg, 25.5 mmol) at 20° C. The mixture was stirred at 60° C. for 3 h. The reaction was diluted with 100 ml water, extracted with 2×50 mL EtOAc. The combined organic layers were washed with 50 mL brine, dried over Na₂SO₄ and filtered. The filtrate was concentrated under reduced pressure to give a residue, which was purified by prep-HPLC (water(0.225% FA)-CAN 16%-46%), and lyophilized to give a residue. The residue was further purified by prep-HPLC (Hexane-EtOH: 5%-45%). The purified solution was concentrated under reduced pressure to give the title compound (630 mg, 1.87 mmol, 22% yield) as a yellow oil. MS (ESI): m/z=337.2 [M+H]⁺

### Example B.550

### 2-(azetidin-3-yl)-5-[[1-(trifluoromethyl)cyclopropyl]methoxy]pyrazine; 4-methylbenzenesulfonic Acid

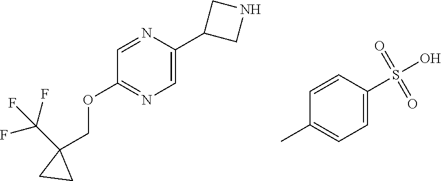 [SMILES: FC(F)(F)C1(COc2cnc(C3CNC3)cn2)CC1 ; Cc1ccc(S(=O)(=O)O)cc1]

To a stirred solution of tert-butyl 3-[5-[[1-(trifluoromethyl)cyclopropyl]methoxy]pyrazin-2-yl]azetidine-1-carboxylate (1550 mg, 4.15 mmol) in EtOAc (70 mL), p-toluenesulfonic acid monohydrate (1974 mg, 10.4 mmol) was added. Then RM was stirred at 35° C. for 18 h. The obtained precipitate was filtered, washed with TBME and dried to give the title compound (1530 mg, 2.48 mmol, 57% yield) as white solid. MS (ESI): m/z=274.2 [M+H]⁺.

### Step a) 2-bromo-5-[[1-(trifluoromethyl)cyclopropyl]methoxy]pyrazine

To a solution of [1-(trifluoromethyl)cyclopropyl]methanol (3.0 g, 21.4 mmol) and 2-bromo-5-fluoro-pyrazine (4.17 g, 23.6 mmol) in THE (50 mL) cooled in ice/water, was added potassium tert-butoxide (3.12 g, 27.8 mmol) in one portion. The reaction mixture was stirred at 20° C. for 12 h. The reaction mixture was concentrated under reduced pressure to give a residue. The residue was diluted with water (100 mL) and then extracted with ethyl acetate (30 mL×3). The combined organic layers were washed with brine (50 mL), dried over Na₂SO₄, filtered and concentrated under reduced pressure to give the crude product. The crude product was purified by flash chromatography on silica gel eluting with PE/EtOAc (15:1) to afford the title compound (4.86 g, 16.4 mmol, 76% yield) as a yellow solid. MS (ESI): m/z=297.0 [M+H]⁺.

### Step b) Tert-Butyl 3-[5-[[1-(trifluoromethyl)cyclopropyl]methoxy]pyrazin-2-yl]azetidine-1-carboxylate

Three batches were set up in parallel. To a mixture of zinc (330 mg, 5.05 mmol) in DMA (8 mL) was added 1,2-dibromoethane (63.2 mg, 0.34 mmol) and CHLOROTRIMETHYLSILANE (36.6 mg, 0.34 mmol). The mixture was heated to 60° C. and stirred for 15 min. Then a mixture of 1-Boc-3-iodoazetidine (1000 mg, 3.53 mmol) in DMA (8 mL) was added. The mixture was stirred for another 15 min. The mixture was cooled to 20° C. and 2-bromo-5-[[1-(trifluoromethyl)cyclopropyl]methoxy]pyrazine (1000 mg, 3.37 mmol), 1,1-BIS(DIPHENYLPHOSPHINO)FERROCENE-PALLADIUM(II)DICHLORIDEDICHLOROMETHANECOMPLEX (137 mg, 0.17 mmol) and COPPER(I) IODIDE (0.01 mL, 0.17 mmol) were added. The mixture was heated to 80° C. and stirred for 12 h. The reaction mixture was filtered and the filter was concentrated to give a residue. The residue was extracted with ethyl acetate (100 mL×3). The combined organic phase was washed with brine (100 mL×3), dried with anhydrous Na₂SO₄, filtered and concentrated in vacuum. The residue was purified by prep-HPLC (water(0.225% FA)-ACN) and lyophilized to give a crude product. Two separate reactions combined for further purification. The combined crude product was purified by silica gel chromatography (0-30% Ethyl acetate/Petroleum ether) and concentrated to give the title compound (1.69 g, 4.53 mmol, 45% yield) as yellow solid. MS (ESI): m/z=318.1 [M-C₄H₈+H]⁺; ¹H NMR (400 MHz, CHLOROFORM-d) δ=8.29 (d, J=1.5 Hz, 1H), 7.93 (d, J=1.0 Hz, 1H), 4.45 (s, 2H), 4.33-4.24 (m, 2H), 4.14-4.11 (m, 2H), 3.85 (tt, J=6.0, 8.8 Hz, 1H), 1.47 (s, 9H), 1.20-1.13 (m, 2H), 0.96-0.91 (m, 2H)

### Example B.551

### 1-(2-azaspiro[3.3]heptan-6-ylmethyl)-5-trifluoromethyl)pyrazin-2-one; 2,2,2-trifluoroacetic Acid

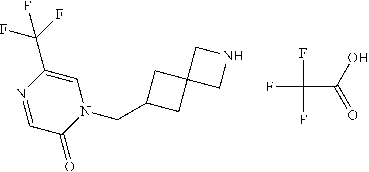 [SMILES: O=c1cnc(C(F)(F)F)cn1CC1CC2(CNC2)C1 ; O=C(O)C(F)(F)F]

To a solution of 6-[[2-keto-5-(trifluoromethyl)pyrazin-1-yl]methyl]-2-azaspiro[3.3]heptane-2-carboxylic acid tert-butyl ester (80 mg, 214 μmol) in dichloromethane (715 μL) was added TFA (244. mg, 165 μL, 2.14 mmol) and the reaction mixture was stirred at RT for 18 h. Volatiles were removed in vacuo to give 161 mg of the crude title compound as a light yellow viscous oil, purity-50%, major contaminant excess of TFA. The crude was used without further purification. MS (ESI): m/z=274.2 [M-TFA+H]⁺.

### Step a) Tert-Butyl 6-[[2-oxo-5-(trifluoromethyl)pyrazin-1-yl]methylene]-2-azaspiro[3.3]heptane-2-carboxylate

To a solution of tert-butyl 6-[(4,4,5,5-tetramethyl-1,3,2-dioxaborolan-2-yl)methylene]-2-azaspiro[3.3]heptane-2-carboxylate (B.1, Step a) (3.34 g, 9.95 mmol), 5-(trifluoromethyl)pyrazin-2-ol (CAS: 5-(trifluoromethyl)pyrazin-2-ol) (1.48 g, 9.04 mmol), boric acid (559 mg, 9.04 mmol) and pyridine (2.19 mL, 27.1 mmol) in DCE (20 mL) was added copper diacetate (3.61 g, 18.1 mmol) under O₂ atmosphere. The reaction mixture was stirred at 70° C. for 12 h under O2 (balloon) condition, and concentrated under reduced pressure to give a residue. The residue was purified by reversed-phase HPLC(0.1% FA condition, MeOH/H₂O=75%) and lyophilized to give a crude product was triturated with petroleum ether (10 mL) at 25° C. for 30 min to give the title compound (1.34 g, 3.61 mmol, 40% yield) as white solid. MS (ESI): m/z=316.2 [M-C₄H₈+H]⁺; ¹H NMR (400 MHz, CHLOROFORM-d) δ=8.20 (s, 1H), 7.55 (s, 1H), 6.80 (br d, J=2.1 Hz, 1H), 4.00 (s, 4H), 3.11 (br s, 4H), 1.44 (s, 9H)

### Step b) Tert-Butyl 6-[[6-oxo-3-(trifluoromethyl)-2,3-dihydropyrazin-1-yl]methyl]-2-azaspiro[3.3]heptane-2-carboxylate

To a solution of tert-butyl 6-[[2-oxo-5-(trifluoromethyl)pyrazin-1-yl]methylene]-2-azaspiro[3.3]heptane-2-carboxylate (1.5 g, 4.04 mmol) in ethyl acetate (20 mL) was added Pd/C (wet) (500 mg) at 25° C. Then the reaction was stirred at 25° C. under H₂ atmosphere (balloon) for 2 h. The reaction mixture was filtered and the filtrate was concentrated under reduced pressure to give tert-butyl 6-[[6-oxo-3-(trifluoromethyl)-2,3-dihydropyrazin-1-yl]methyl]-2-azaspiro[3.3]heptane-2-carboxylate (600.0 mg, 1.6 mmol, 39% yield). MS (ESI): m/z=276.1 [M−Boc+H]⁺ and tert-butyl 6-[[2-oxo-5-(trifluoromethyl)piperazin-1-yl]methyl]-2-azaspiro[3.3]heptane-2-carboxylate (600.0 mg, 1.59 mmol, 40% yield) MS (ESI): m/z=278.1 [M−Boc+H]⁺ as brown solid.

Note: over hydrogenation of the pyrazine ring compared to standard synthesis. Both products carried into the next step, where it was possible to re-oxidize the dihydropyrazin-1-yl compound back to the pyrazine (piperazin-1-yl compound recovered unreacted).

### Step c) 6-[[2-keto-5-(trifluoromethyl)pyrazin-1 yl]methyl]-2-azaspiro[3.3]heptane-2-carboxylic Acid Tert-Butyl Ester

To the mixture of tert-butyl 6-[[2-oxo-5-(trifluoromethyl)piperazin-1-yl]methyl]-2-azaspiro[3.3]heptane-2-carboxylate (500 mg, 1.32 mmol) in DCE (15 mL) was added MnO₂ (4.61 g, 53.0 mmol), then the reaction mixture was stirred at 20° C. for 12 h. The reaction mixture was filtered and the filtrate was concentrated under reduced pressure to give residue. The residue was purified by prep-HPLC (water(FA)-ACN) and lyophilized to give the title compound (79.8 mg, 0.21 mmol, 16% yield) as a yellow solid. MS (ESI): m/z=318.0 [M-C₄H₈+H]⁺; 274.0 [M−Boc+H]⁺; ¹H NMR (400 MHz, CHLOROFORM-d) δ=8.16 (s, 1H), 7.50 (s, 1H), 3.95-3.92 (m, 4H), 3.87 (s, 2H), 2.70-2.59 (m, 1H), 2.38-2.29 (m, 2H), 2.05-1.96 (m, 2H), 1.43 (s, 9H), 1.26 (s, 1H)

### Example B.552

### 6-[[5-(trifluoromethyl)-2-pyridyl]oxymethyl]-2-azaspiro[3.3]heptane; 4-methylbenzenesulfonic Acid

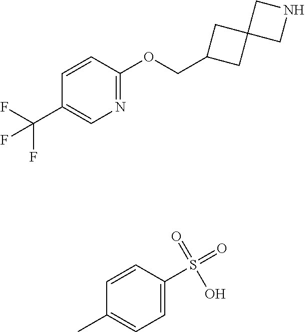 [SMILES: FC(F)(F)c1ccc(OCC2CC3(CNC3)C2)nc1 ; Cc1ccc(S(=O)(=O)O)cc1]

To a solution of tert-butyl 6-[[5-(trifluoromethyl)-2-pyridyl]oxymethyl]-2-azaspiro[3.3]heptane-2-carboxylate (1299 mg, 3.49 mmol) in EtOAc (21.7 mL) p-toluenesulfonic acid monohydrate (1991 mg, 10.5 mmol) was added. Reaction mixture was stirred at 25° C. for 72 h, then filtered and washed with ether to give the title compound (1538 mg, 3.46 mmol, 94% yield) as white solid. MS (ESI): m/z=273.0 [M+H]⁺.

### Step a) Tert-Butyl 6-[[5-(trifluoromethyl)-2 pyridyl]oxymethyl]-2-azaspiro[3.3]heptane-2-carboxylate

To a solution of 5-(trifluoromethyl)-2-pyridone (294 mg, 1.8 mmol) in dry N,N-dimethylformamide (7.5 mL) cooled down to 0° C. under an inert atmosphere was added NaH (60% in mineral oil) (72 mg, 1.8 mmol) and the reaction mixture was stirred at 0° C. for 10 min and 15 mm at RT followed by addition of 6-(methylsulfonyloxymethyl)-2-azaspiro[3.3]heptane-2-carboxylic acid tert-butyl ester (P.23, Step a) (500 mg, 1.64 mmol) after which the reaction mixture was stirred at RT for 18 h. Volatiles were removed in vacuo, the crude residue was partitioned between ethyl acetate and aq. Na₂CO₃ 1 M (aq.). The organic phase was collected and the aqueous phase was back-extracted with ethyl acetate. The combined organic phases were dried over sodium sulfate and evaporated down to dryness to give a residue. The crude material was purified by flash chromatography, eluent mixture of heptane and ethyl acetate (5% to 80%) to give the title compound (227 mg) as a white solid. MS (ESI): m/z=317.2 [M-C₄H₈+H]⁺. Also isolated was regioisomeric side product 6-[[2-keto-5-(trifluoromethyl)-1-pyridyl]methyl]-2-azaspiro[3.3]heptane-2-carboxylic acid tert-butyl ester (precursor, P.24) (266 mg); MS (ESI): m/z=317.2 [M-C₄H₈+H]⁺.

### Example B.553

### 4-[[5-(Trifluoromethyl)pyrazin-2-yl]amino]cubane-1-carboxylic Acid

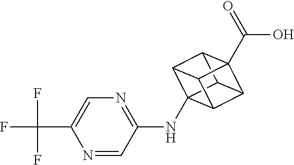 [SMILES: O=C(O)C12C3C4C1C1C2C3C41Nc1cnc(C(F)(F)F)cn1]

To a solution of 4-[[5-(trifluoromethyl)pyrazin-2-yl]amino]cubane-1-carboxylic acid methyl ester (52 mg, 0.153 mmol) in THE (1.31 mL) was added water (0.218 mL) and LiOH monohydrate (19.24 mg, 0.458 mmol). The mixture was sonicated and allowed to stir for 1 h. EtOAc was added and 2 M HCl (aq.). The phases were separated and the organic layer was washed with brine, dried over Na₂SO₄, filtered and evaporated under reduced pressure to get the title compound as a yellow solid (36.5 mg, 73.3%). MS (ESI): m/z=308.1 [M−H]⁻.

### Step a) Methyl 4-[[5-(trifluoromethyl)pyrazin-2-yl]amino]cubane-1-carboxylate

To a solution of 4-(tert-butoxycarbonylamino)cubane-1-carboxylic acid methyl ester (42.3 mg, 0.153 mmol, CAS RN: 883554-71-0) in DCM (0.423 mL) was added TFA (423 μL, 5.49 mmol) and the reaction was allowed to stir for 1 h. Then, TFA and DCM were evaporated under reduced pressure (TFA azeotropically removed with 3×1 mL DCM). The residue was taken up in DMF, extra dry (1.53 mL). Then, DIEA (59.1 mg, 79.9 μL, 0.458 mmol) was added as well as 2-fluoro-5-(trifluoromethyl)pyrazine (27.8 μL, 0.229 mmol). The reaction vessel was flushed with nitrogen before being sealed and allowed to stir over night. The reaction mixture was diluted with DCM and water. The phases were separated and the aqueous layer was extracted with DCM. The combined organic layers were washed with brine, dried over Na₂SO₄, filtered and evaporated under reduced pressure to get the desire product as a yellow gum (52 mg, 100%). MS (ESI): m/z=324.1 [M+H]⁺.

### Example B.554

### 4-[[2-Fluoro-4-(trifluoromethoxy)phenyl]methoxy]piperidine; Hydrochloride Salt

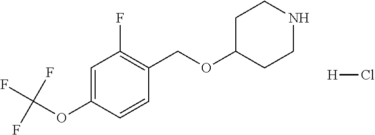

To a solution of tert-butyl 4-[[2-fluoro-4-(trifluoromethoxy)phenyl]methoxy]piperidine-1-carboxylate (750 mg, 1.91 mmol) in DCM (11.8 mL) and methanol (2.35 mL), 4 M HCl (4 M in Dioxane) (753 μL, 3.01 mmol) was added and the mixture was stirred overnight at RT. The mixture was evaporated to give the title compound as a colorless solid (0.5 g, 80.4%). The product was used as is for next step.

### Step a) Tert-Butyl 4-[[2-fluoro-4-(trifluoromethoxy)phenyl]methoxy]piperidine-1-carboxylate

To a solution of 4-hydroxypiperidine-1-carboxylic acid tert-butyl ester (1.5 g, 7.45 mmol, CAS RN 109384-19-2) in dry THF (3 mL) was added potassium tert-butoxide 1M solution in THF (1.04 ml, 1.04 mmol) and the turbid reaction mixture was stirred at RT for 15 min followed by addition of 1-(bromomethyl)-2-chloro-4-fluorobenzene (222 mg, 994 μcool, CAS RN 1240257-47-9). The reaction mixture was then stirred at RT for 3 hours. The reaction mixture was poured on water and ethyl acetate and the layers were separated. The organic layer was washed twice with water and dried over MgSO₄, filtered and evaporated to give the desired product as a colorless oil (2.7 g, 73.6%). The product was used as is for next step.

### Example M.1

### 3-[3-(4-Chloro-2-methylsulfonyl-phenyl)-1-bicyclo[1.1.1]pentanyl]azetidine; 4-methylbenzenesulfonic Acid

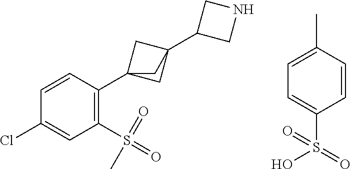 [SMILES: CS(=O)(=O)c1cc(Cl)ccc1C12CC(C3CNC3)(C1)C2 ; Cc1ccc(S(=O)(=O)O)cc1]

To a solution of 3-[3-(4-chloro-2-mesyl-phenyl)-1-bicyclo[1.1.1]pentanyl]azetidine-1-carboxylic acid tert-butyl ester (161 mg, 0.391 mmol) in ethyl acetate (1.5 mL) was added p-toluenesulfonic acid monohydrate (89.21 mg, 0.469 mmol) and the mixture was stirred at reflux for 1.5 h. After cooling down to 23° C., the solution was stirred at this temperature for 2 h, before being evaporated to give the title compound (0.222 g, 99.7%) as a colorless foam. MS (ESI): m/z=312.0 [M+H]⁺

### Step a): 3-[3-(4-chloro-2-fluoro-phenyl)-1-bicyclo[1.1.1]pentanyl]azetidine-1-carboxylic Acid Tert-Butyl Ester

In a sealed and argon filled tube were combined 3-(3-iodo-1-bicyclo[1.1.1]pentanyl)azetidine-1-carboxylic acid tert-butyl ester (CAS RN: 2375261-02-0; 410 mg, 1.17 mmol), 1-bromo-4-chloro-2-fluoro-benzene (219.81 μL, 1.76 mmol), dichloronickel, 1,2-dimethoxyethane (25.8 mg, 0.117 mmol), bis[3,5-difluoro-2-[5-(trifluoromethyl)-2-pyridyl]phenyl]iridium(1+); 4-tert-butyl-2-(4-tert-butyl-2-pyridyl)pyridine; hexafluorophosphate (13.17 mg, 0.012 mmol) and 4-tert-butyl-2-(4-tert-butyl-2-pyridyl)pyridine (31.51 mg, 0.117 mmol) and the mixture was suspended in ethylene glycol dimethyl ether (12 mL) before addition of bis(trimethylsilyl)silyl-trimethyl-silane (362.22 μL, 1.17 mmol) and 2,6-dimethylpyridine (251.61 mg, 2.35 mmol). After degassing with argon, the suspension was stirred in a photoreactor with a 420 nm lamp (25% intensity) for 21.5 h. The mixture was diluted with methanol, treated with silica gel and evaporated. Purification by FC (SiO₂; heptane/EtOAc) gave the title compound (0.091 g, 22.0%) as a colorless oil. MS (ESI): m/z=296.0 [M-C4H8+H]⁺.

### Step b): Tert-Butyl 3-[3-(4-chloro-2-methylsulfonyl phenyl)-1-bicyclo[1.1.1]pentanyl]azetidine-1-carboxylate

To a solution of tert-butyl 3-[3-(4-chloro-2-fluoro-phenyl)-1-bicyclo[1.1.1]pentanyl]azetidine-1-carboxylate (300 mg, 0.767 mmol) in dimethyl sulfoxide (2.5 mL) was added NaSMe (80.68 mg, 1.15 mmol) and the mixture was stirred at 100° C. for 1 hour. The mixture was allowed to cool down to RT. The reaction mixture was poured on water and ethyl acetate and the layers were separated. The aqueous layer was extracted twice with ethyl acetate. The organic layers were washed twice with water, dried over MgSO4, filtered and evaporated. The crude intermediate was dissolved in methanol (1.8 mL), water (1.8 mL) and oxone (990.7 mg, 1.61 mmol) and NaHCO₃ (154.73 mg, 1.84 mmol) were added. After 2.75 hours another batch of oxone (235.88 mg, 0.384 mmol) and NaHCO₃ (32.23 mg, 0.384 mmol) was added. The mixture was stirred overnight. The mixture was diluted with saturated aqueous NaHCO₃ solution and ethyl acetate and the layers were separated. The aqueous layer was extracted twice with ethyl acetate. The organic layers were washed once with water, dried over MgSO₄, filtered and evaporated. Purification by FC (SiO₂; heptane/EtOAc) gave the title compound (0.135 g, 42.7%) as a colorless gum. MS (ESI): m/z=356.0 [M-C₄H₈+H]⁺

In analogy to Example M. 1, the following building block was generated using the relevant commercial building block in Step a).

| | | | Building | MS, ESI: |
|---|---|---|---|---|
| Ex. | Structure | Systematic Name | Blocks | m/z |
| M.11 *depro- tec- tion with TFA | 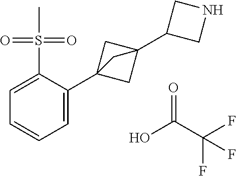 | 3-[3-(2-mesylphenyl)- 1- bicyclo[1.1.1]pentanyl] azetidine .1:1 2,2,2- trifluoroacetic acid | bromo-[2- (methylthio) phenyl] magnesium 0.5M solution in THF | 278.1 [M + H]⁺ |

### Example M.3

### 2-[3-(azetidin-3-yl)-1-bicyclo[1.1.1]pentanyl]-5-(2,2-dimethylpropyl)-1,3,4-thiadiazole; 4-methylbenzenesulfonic Acid

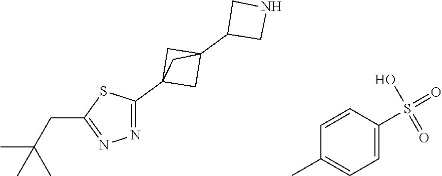 [SMILES: CC(C)(C)Cc1nnc(C23CC(C4CNC4)(C2)C3)s1 ; Cc1ccc(S(=O)(=O)O)cc1]

A solution of tert-butyl 3-[3-[5-(2,2-dimethylpropyl)-1,3,4-thiadiazol-2-yl]-1-bicyclo[1.1.1]pentanyl]azetidine-1-carboxylate (888.0 mg, 2.35 mmol) and p-toluenesulfonic acid monohydrate (559.26 mg, 2.94 mmol) in Methanol (30 mL) was stirred at 20° C. for 72 h, before being evaporated. The residue was treated with Et₂O (50 mL) and stirred for 18 h. The resulting precipitate was filtered, washed with Et₂O and dried in vacuo to give the title compound (830.0 mg, 70.63% yield) as a white solid. MS (ES): m/z=278.2 [M+H]⁺

### Step a): Tert-Butyl 3-[3-[(3,3-dimethylbutanoylamino)carbamoyl]-1-bicyclo[1.1.1]pentanyl]azetidine-1-carboxylate

A solution of 3,3-dimethylbutanehydrazide (CAS RN: 712303-26-9; 1.95 g, 14.96 mmol) in DMF (100 mL) was treated with HATU (7.39 g, 19.45 mmol) and DIPEA (7.73 g, 59.85 mmol), and was stirred for 10 min at 23° C. 3-(1-Tert-butoxycarbonylazetidin-3-yl)bicyclo[1.1.1]pentane-1-carboxylic acid (CAS RN: 1211526-53-2; 4.0 g, 14.96 mmol) was added in one portion and the mixture was stirred for another 23 h at 23° C., before being poured onto water (300 mL) and extracted with TBME (3×100 mL). The combined organic layers were successively washed with water (50 mL) and brine (50 mL), dried over Na₂SO₄, filtered and co-evaporated with toluene (100 mL), to give the title compound (5.2 g, 87.0% yield) as a light yellow solid. MS (ES): m/z=378.2 [M−H]⁻

### Step b): Tert-Butyl 3-[3-[5-(2,2-dimethylpropyl)-1,3,4-thiadiazol-2 yl]-1-bicyclo[1.1.1]pentanyl]azetidine-1-carboxylate

A solution of tert-butyl 3-[3-[(3,3-dimethylbutanoylamino)carbamoyl]-1-bicyclo[1.1.1]pentanyl]azetidine-1-carboxylate (5.2 g, 13.7 mmol) in toluene (250 mL) was added Lawesson's reagent (6.65 g, 16.44 mmol). The mixture was stirred 80° C. for 18 h, before being cooled down. The reaction mixture was washed with aqueous sodium bicarbonate solution (150 mL) followed by water (30 mL). The aqueous layer was extracted with TBME (2×100 mL). The combined organic layers were washed with water (100 mL) and brine (100 mL), dried over anhydrous sodium sulfate and evaporated. Purification by FC (SiO₂; PE/MTBE) gave the title compound (888.0 mg, 16.31% yield) as a white solid. MS (ESI): m/z=378.2 [M+H]⁺

In analogy to Example M.3, the following building block was generated using the relevant commercial building block in Step a).

| | | | Building | MS, ESI: |
|---|---|---|---|---|
| Ex. | Structure | Systematic Name | Blocks | m/z |
| M.6 *depro- tec- tion with TFA | 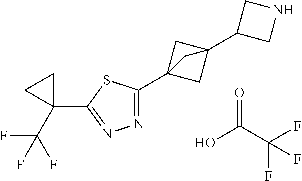 | 2-[3-(azetidin-3-yl)-1- bicyclo[1.1.1]pentanyl]- 5-[1- (trifluoromethyl) cyclopropyl]-1,3,4- thiadiazole; 2,2,2- trifluoroacetic acid | 1-(trifluoro- methyl) cyclopropane- 1-carbo- hydrazide (CAS RN: 1016557-86-0) | 316.2 [M + H]⁺ |

### Example M.4

### 1: 3-[3-(4-triflylphenyl)-1-bicyclo[1.1.1]pentanyl]azetidine.1:1 2,2,2-trifluoroacetic Acid

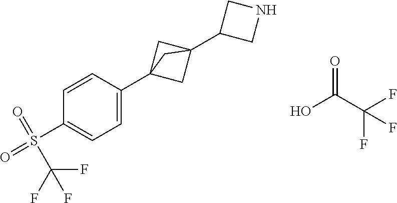 [SMILES: O=S(=O)(c1ccc(C23CC(C4CNC4)(C2)C3)cc1)C(F)(F)F ; O=C(O)C(F)(F)F]

A solution of 3-[3-(4-triflylphenyl)-1-bicyclo[1.1.1]pentanyl]azetidine-1-carboxylic acid tert-butyl ester (435 mg, 957.77 μmot) in dichloromethane (4 mL) was treated with TFA (737.87 μL, 9.58 mmol), at 23° C. The mixture was stirred for 18 h at this temperature before being evaporated, to give the title compound (593 mg, quant.) as a light yellow viscous oil. MS (ESI): m/z=332.0 [M+H]⁺

### Step a): 3-(3-phthalimidooxycarbonyl-1-bicyclo[1.1.1]pentanyl)azetidine-1-carboxylic Acid Tert-Butyl Ester

A solution of 3-(1-tert-butoxycarbonylazetidin-3-yl)bicyclo[1.1.1]pentane-1-carboxylic acid (CAS RN: 1211526-53-2; 5 g, 18.7 mmol) in dichloromethane (92 mL) was treated with 2-hydroxyisoindoline-1,3-quinone (3.36 g. 20.57 mmol), DCC (4.25 g, 20.57 mmol) and DMAP (228.51 mg, 1.87 mmol), at 23° C. The mixture was stirred for 18 h, before being evaporated. Purification by FC (SiO₂; CH₂Cl₂/EtOAc) gave the title compound (7.28 g, 92.5% yield) as a white solid. MS (ESI): m/z=357.1 [M-tBu+H]⁺

### Step b): 3-[3-(4-triflylphenyl)-1-bicyclo[1.1.1]pentanyl]azetidine-1-carboxylic Acid Tert-Butyl Ester

A MW vial was charged with 3-(3-phthalimidooxycarbonyl-1-bicyclo[1.1.1]pentanyl)azetidine-1-carboxylic acid tert-butyl ester (800 mg, 1.84 mmol), 1-bromo-4-triflyl-benzene (1.07 g, 3.69 mmol), Ni(dtbbpy)Br₂ (179.44 mg, 368.54 μmol), NaHCO₃ (619.14 mg, 7.37 mmol) and 2,6-dimethyl-1,4-dihydropyridine-3,5-dicarboxylic acid diethyl ester (933.47 mg, 3.69 mmol), put under Ar, and sealed. N,N-dimethylacetamide, extra dry (18.41 mL) was added, and the mixture was stirred in a photoreactor under 395 nm LED irradiation (stirring 1000 rpm, fan speed 6800, LED lamp intensity 50%) for 16 h at 23° C., before being evaporated. The residue was partitioned between TBME and aqeuous 1 M NaOH. The organic layer was collected and the aqueous phase was back-extracted with TBME. The combined organic layers were dried over sodium sulfate and evaporated. Purification by FC (SiO₂; heptane/MTBE) gave the title compound (463 mg, 52.1% yield) as a light yellow viscous oil. MS (ESI): m/z=376.1 [M-tBu+H]⁺

In analogy to Example M.4, the following building block was generated using the relevant commercial building block in Step b).

| | | | Building | MS, ESI: |
|---|---|---|---|---|
| Ex. | Structure | Systematic Name | Blocks | m/z |
| M.9 | 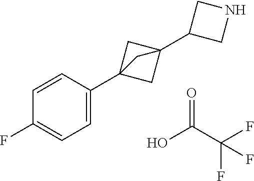 | 3-[3-(4-fluorophenyl)- 1- bicyclo[1.1.1]pentanyl] azetidine; 2,2,2- trifluoroacetic acid | 1-Bromo-4- fluorobenzene (CAS RN: 460-00-4) | 218.1 [M + H]⁺ |
| M.27 | 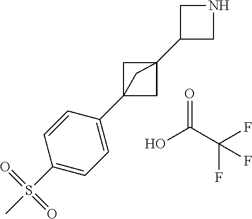 | 3-[3-(4- methylsulfonylphenyl)- 1-bicyclo[1.1.1]- pentanyl]azetidine; 2,2,2- trifluoroacetic acid | 1-bromo-4- mesyl-benzene (CAS RN: 3466-32-8) | 278.1 [M + H]⁺ |
| M.28 | 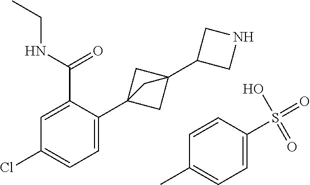 | 2-[3-(azetidin-3-yl)-1- bicyclo[1.1.1]pentanyl]- 5-chloro-N-ethyl- benzamide; 4- methylbenzenesulfonic acid | 2-bromo-5- chloro-N- ethyl- benzamide (CAS RN: 150079-87-1) | 305.1 [M + H]⁺ |
| | *deprotection with PTSA | | | |
| M.29 | 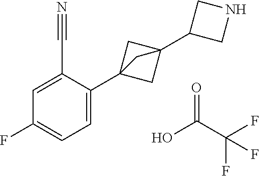 | 2-[3-(azetidin-3-yl)-1- bicyclo[1.1.1]pentanyl]- 5-fluoro- benzonitrile; 2,2,2- trifluoroacetic acid | 2-bromo-5- fluoro- benzonitrile (CAS RN: 57381-39-2) | 243.2 [M + H]⁺ |
| M.31 | 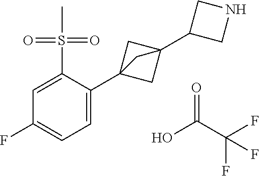 | 3-[3-(4-fluoro-2- methylsulfonyl- phenyl)-1- bicyclo[1.1.1]- pentanyl]azetidine; 2,2,2- trifluoroacetic acid | 1-bromo-4- fluoro-2- mesyl-benzene (CAS RN: 628692-10-4) | 296.1 [M + H]⁺ |

### Example M.5

### 3-[3-[4-(trifluoromethoxy)phenyl]-1-bicyclo[1.1.1]pentanyl]azetidine 0.1:1 2,2,2-trifluoroacetic Acid

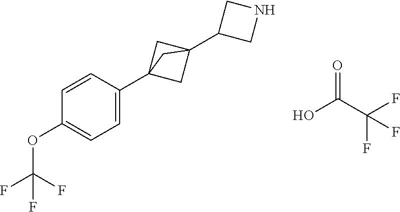 [SMILES: FC(F)(F)Oc1ccc(C23CC(C4CNC4)(C2)C3)cc1 ; O=C(O)C(F)(F)F]

A solution of 3-[3-[4-(trifluoromethoxy)phenyl]-1-bicyclo[1.1.1]pentanyl]azetidine-1-carboxylic acid tert-butyl ester (178 mg, 0.464 mmol) in dichloromethane (2 mL) was treated with TFA (357.67 μL, 4.64 mmol) and stirred for 18 h at 23° C. before being evaporated, to give the crude title compound (268 mg, quart.). MS (ESI): m/z=284.1 [M+H]⁺

### Step a): 3-[3-[4-(trifluoromethoxy)phenyl]-1-bicyclo[1.1.1]pentanyl]azetidine-1-carboxylic Acid Tert-Butyl Ester

A flask was charged with 3-(3-iodo-1-bicyclo[1.1.1]pentanyl)azetidine-1-carboxylic acid tert-butyl ester (CAS RN: 2375261-02-0; 500 mg, 1.43 mmol) and Fe(acac)₃ (101.13 mg, 0.286 mmol) after which the atmosphere was switched to an argon atmosphere followed by addition of tetrahydrofuran, extra dry (1.5 mL) and TMEDA (85.9 μL, 0.573 mmol). The mixture was treated dropwise with 0.500 M bromo-[4-(trifluoromethoxy)phenyl]magnesium (4.58 mL, 2.29 mmol) with a syringe pump over 60 min after which the reaction was stirred at r.t for 2 h, before being treated with of a few drops of sat. aq. NH₄Cl and stirred at 23° C. for 5 min. The mixture was poured into a separating funnel containing ethyl acetate and sat. aq. NH₄Cl. The organic layer was collected and the aqueous layer was back-extracted with ethyl acetate. The combined organic layers were dried over sodium sulfate and evaporated. Purification by FC (SiO₂; heptane/TBME) and RP-HPLC gave the title compound as a colorless gum (178 mg, 31% yield). MS (ESI): m/z=328.1 [M-tBu+H]⁺

In analogy to Example M.5, the following building block was generated using the relevant commercial building block in Step a).

| | | | Building | MS, ESI: |
|---|---|---|---|---|
| Ex. | Structure | Systematic Name | Blocks | m/z |
| M.8 | 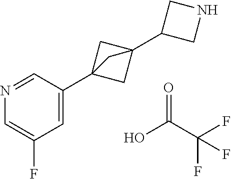 | 3-[3-(azetidin-3-yl)-1- bicyclo[1.1.1]pentanyl]- 5-fluoro-pyridine .1:1 2,2,2- trifluoroacetic acid | 0.250M bromo-(5- fluoro-3- pyridyl) magnesium in 2-Me- THF | 219.1 [M + H]⁺ |
| M.26 | 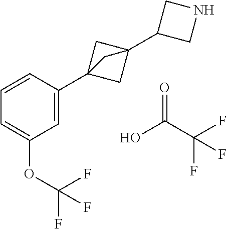 | 2,2,2-trifluoroacetic acid; 3-[3-[3- (trifluoromethoxy) phenyl]-1- bicyclo[1.1.1]pentanyl] azetidine | 0.5M bromo- [3- (trifluoro- methoxy) phenyl]mag- nesium in THF | 284.1 [M + H]⁺ |
| M.30 | 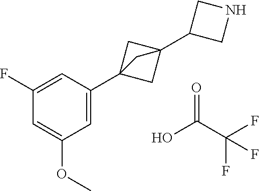 | 3-[3-(3-fluoro-5- methoxy-phenyl)-1- bicyclo[1.1.1]pentanyl] azetidine; 2,2,2- trifluoroacetic acid | 0.5M bromo- (3-fluoro-5- methoxy- phenyl)magne- sium in THF | 248.2 [M + H]⁺ |

### Example M.7

### 5-[[3-(azetidin-3-yl)-1-bicyclo[1.1.1]pentanyl]methyl]-2-(trifluoromethyl)pyridine; 4-methylbenzenesulfonic Acid

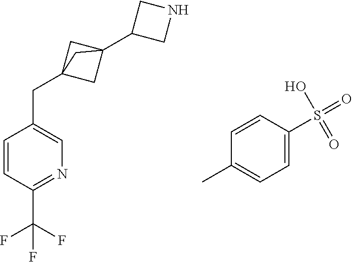 [SMILES: FC(F)(F)c1ccc(CC23CC(C4CNC4)(C2)C3)cn1 ; Cc1ccc(S(=O)(=O)O)cc1]

p-Toluenesulfonic acid monohydrate (525.27 mg, 2.76 mmol) was added to a stirred solution of tert-butyl 3-[3-[[6-(trifluoromethyl)-3-pyridyl]methyl]-1-bicyclo[1.1.1]pentanyl]azetidine-1-carboxylate (480.0 mg, 1.26 mmol) in EtOAc (25 mL). The mixture was stirred at 25° C. for 16 h before being evaporated, to give the title compound (465.2 mg, 77.47% yield) as a light yellow solid. MS (ESI): m/z=283.2 [M+H]⁺

### Step a): 4-methyl-N—[(E)-[6-(trifluoromethyl)-3-pyridyl]methyleneamino]benzenesulfonamide

A solution of 6-(trifluoromethyl)pyridine-3-carboxaldehyde (CAS RN: 386704-12-7; 2.7 g, 15.42 mmol) and 4-methylbenzenesulfonhydrazide (3158.6 mg, 16.96 mmol) in MeOH (30 mL) was refluxed for 16 h, before being cooled down and evaporated. Trituration with Et₂O and hexanes gave the title compound (5.2 g, 88.41% yield) as a yellow solid. MS (ESI): m/z=344.0 [M+H]⁺

### Step b): Tert-Butyl 3-[3-[[6-(trifluoromethyl)-3 pyridyl]methyl]-1-bicyclo[1.1.1]pentanyl]azetidine-1-carboxylate

A solution of [3-(1-tert-butoxycarbonylazetidin-3-yl)-1-bicyclo[1.1.1]pentanyl]boronic acid (2.22 g, 8.3 mmol), 4-methyl-N—[(E)-[6-(trifluoromethyl)-3-pyridyl]methyleneamino]benzenesulfonamide (1.9 g, 5.53 mmol) and cesium carbonate (2.7 g, 8.3 mmol) in dry 1,4-dioxane (50 mL) were refluxed for 48 h under Ar. The resulting precipitate was filtered off and the filtrate was evaporated. The residue was partitioned between TBME (200 mL) and water (50 mL). The organic layer was washed with brine (20 mL), dried over Na₂SO₄ and evaporated. Purification by FC (SiO₂; PE/MTBE) gave the title compound (580.0 mg, 27.41% yield) as a light yellow oil. MS (ESI): m/z=283.2 [M-Boc+H]⁺

In analogy to Example M.7, the following building block was generated using the relevant commercial building block in Step a).

| | | | Building | MS, ESI: |
|---|---|---|---|---|
| Ex. | Structure | Systematic Name | Blocks | m/z |
| M.23 | 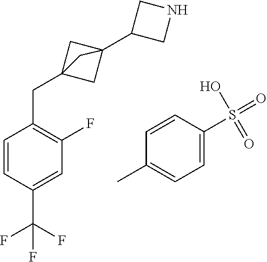 [SMILES: Fc1cc(C(F)(F)F)ccc1CC12CC(C3CNC3)(C1)C2 ; Cc1ccc(S(=O)(=O)O)cc1] | 3-[3-[[2-fluoro-4- (trifluoromethyl) phenyl]methyl]-1- bicyclo[1.1.1]pentanyl] azetidine; 4- methylbenzenesulfonic acid | 2-fluoro-4- (trifluoro- methyl)benzal- dehyde (CAS RN: 89763-93- 9) | 300.2 [M + H]⁺ |

### Example M.10

3-[3-[difluoro-[4-(trifluoromethyl)phenyl]methyl]-1-bicyclo[1.1.1]pentanyl]azetidine; 4-methylbenzenesulfonic Acid

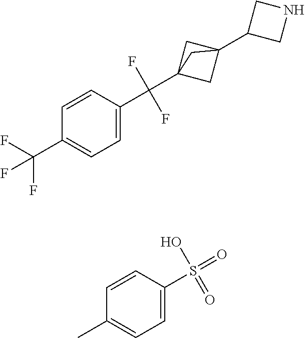 [SMILES: FC(F)(F)c1ccc(C(F)(F)C23CC(C4CNC4)(C2)C3)cc1 ; Cc1ccc(S(=O)(=O)O)cc1]

p-Toluenesulfonic acid monohydrate (0.98 g, 5.17 mmol) was added to a solution of tert-butyl 3-[3-[difluoro-[4-(trifluoromethyl)phenyl]methyl]-1-bicyclo[1.1.1]pentanyl]azetidine-1-carboxylate (1.8 g, 4.31 mmol) in EtOAc (50 mL). The mixture was stirred at 23° C. for 16 h, before being evaporated. Trituration with MTBE gave the title compound (1.65 g, 74.17% yield) as a white solid. MS (ESI): m/z=318.0 [M+H]⁺

### Step a): 3-[3-[methoxy(methyl)carbamoyl]-1-bicyclo[1.1.1]pentanyl]azetidine-1-carboxylic Acid Tert-Butyl Ester

A solution of 3-(1-tert-butoxycarbonylazetidin-3-yl)bicyclo[1.1.1]pentane-1-carboxylic acid (CAS RN: 1211526-53-2; 3 g, 11.22 mmol) in dichloromethane (45 mL) was cooled down to 0° C. and treated with CDI (1.91 g, 11.78 mmol), under Ar. The mixture was stirred for 5 min at this temperature before being allowed to warm up to 23° C. and treated with N,O-dimethylhydroxylamine hydrochloride (1.15 g, 11.78 mmol) and DIPEA (2.94 mL, 16.83 mmol). The mixture was stirred for 18 h at 23° C., and poured in a separating funnel containing dichloromethane and 0.5 M aqueous HCl. After extraction, the organic phase was collected and the aqueous phase was back-extracted with dichloromethane. The combined organic phases were washed with brine, dried over sodium sulfate and evaporated, to give the title compound (3.38 g, quant.) as a crude solid. MS (ESI): m/z=311.3 [M+H]⁺

### Step b): Tert-Butyl 3-[3-[4-(trifluoromethyl)benzoyl]-1-bicyclo[1.1.1]pentanyl]azetidine-1-carboxylate

A solution of tert-butyl 3-[3-[methoxy(methyl)carbamoyl]-1-bicyclo[1.1.1]pentanyl]azetidine-1-carboxylate (3.4 g, 10.95 mmol) in THE (10 mL) at 0° C. was treated with the Grignard reagent (which was prepared from magnesium (665.59 mg, 27.38 mmol) and 4-bromobenzotrifluoride (3.83 mL, 27.38 mmol) and the reaction mixture was stirred at for 48 h at 23° C. before being quenched with aqueous NH₄Cl. The aqueous phase was extracted with EtOAc and the combined organic phases were dried over Na₂SO₄, filtered, and evaporated. Purification by FC gave the title compound (250 mg, 39.25% yield) as a light yellow oil. MS (ESI): m/z=296 [M-boc+H]⁺

### Step c): Tert-Butyl 3-[3-[difluoro-[4-(trifluoromethyl)phenyl]methyl]-1-bicyclo[1.1.1]pentanyl]azetidine-1-carboxylate

A solution of tert-butyl 3-[3-[4-(trifluoromethyl)benzoyl]-1-bicyclo[1.1.1]pentanyl]azetidine-1-carboxylate (3.0 g, 7.59 mmol) in toluene (10 mL) was treated dropwise with a solution of bis(2-methoxyethyl)aminosulfur trifluoride (5.04 g, 22.76 mmol) in toluene (15 mL), at 23° C. The mixture was stirred for 2 d at this temperature, before being cooled down to 0° C. and slowly treated with a saturated aqueous NaHCO₃ solution. The mixture was extracted with EtOAc (3×50 mL). The organic layers were washed with brine, dried over Na₂SO₄, filtered, and evaporated. Purification by FC gave the title compound (2.0 g, 57.47% yield) as a light yellow solid. MS (ESI): m/z=318.2 [M-boc+H]⁺

### Example M.12

### 2-[[3-(azetidin-3-yl)-1-bicyclo[1.1.1]pentanyl]methyl]-5-cyclopropyl-1,3,4-oxadiazole; 2,2,2-trifluoroacetic Acid

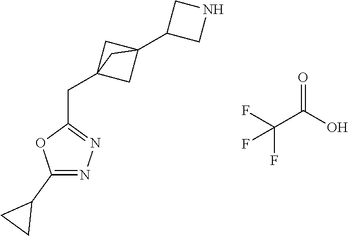 [SMILES: C1CC1c1nnc(CC23CC(C4CNC4)(C2)C3)o1 ; O=C(O)C(F)(F)F]

A solution of tert-butyl 3-[3-[(5-cyclopropyl-1,3,4-oxadiazol-2-yl)methyl]-1-bicyclo[1.1.1]pentanyl]azetidine-1-carboxylate (550.0 mg, 1.59 mmol) and trifluoroacetic acid (0.61 mL, 7.96 mmol) in DCM (50 mL) was stirred at 23° C. for 48 h, before being evaporated. Purification by RP-HPLC gave the title compound (103.1 mg, 16.22% yield) as a colorless viscous oil. MS (ESI): m/z=246.0 [M+H]⁺

### Step a): Tert-Butyl 3-(3-isobuloxycarbonyloxycarbonyl-1-bicyclo[1.1.1]pentanyl)azetidine-1-carboxylate

A solution of 3-(1-tert-butoxycarbonylazetidin-3-yl)bicyclo[1.1.1]pentane-1-carboxylic acid (CAS RN: 2227205-20-9; 5.0 g, 18.7 mmol) and 4-methylmorpholine (2270.31 mg, 22.45 mmol) in THF (150 mL) was treated dropwise with isobutyl chloroformate (2.55 g, 18.7 mmol), at 0° C. The mixture was allowed to warm up to 23° C. and stirred for 1 h at this temperature. A precipitate formed and was filtered off. The filtrate was evaporated, to give the crude title compound (6.1 g, 84.32% yield), which was directly used in the next step.

### Step b): Tert-Butyl 3-[3-(2-diazoacetyl)-1-bicyclo[1.1.1]pentanyl]azetidine-1-carboxylate

A solution of tert-butyl 3-(3-isobutoxycarbonyloxycarbonyl-1-bicyclo[1.1.1]pentanyl)azetidine-1-carboxylate (1400.0 mg, 3.81 mmol) in THF (30 mL) was treated dropwise with a solution of diazomethane (480.54 mg, 11.43 mmol) in TBME (50 mL), at 0° C. The mixture was allowed to warm up to 23° C. and stirred for another 3 h at this temperature, before being carefully treated with 75 mL of 0.5 N acetic acid. Saturated aqueous sodium bicarbonate solution (75 mL) was then added carefully. The organic layer was washed with saturated aqueous sodium chloride (75 mL), dried over magnesium sulfate, filtered, and evaporated. The title compound was used directly in the next step.

Caution Diazomethane should be handled in an efficient fume hood behind a protection shield because of its toxicity and the possibility of explosions.

### Step c): 2-[3-(1-tert-butoxycarbonylazetidin-3-yl)-1-bicyclo[1.1.1]pentanyl]acetic Acid

A 500-mL, three-necked flask was equipped with a nitrogen gas inlet, bubble counter, septum and a magnetic stirring bar. The flask was carefully wrapped in aluminum foil (to exclude light during the reaction). The crude diazo ketone from the preceding step was dissolved in THF (380 mL) and added to the flask under an atmosphere of nitrogen. De-ionized water (38 mL) was added, the flask was immersed in a dry ice-acetone bath, and the solution was cooled to −25° C. (temperature of the acetone cooling bath) for 30 min. Silver trifluoroacetate (2.72 g, 12.3 mmol) was placed in a 50-mL Erlenmeyer flask and quickly dissolved in triethylamine (39 mL, 279 mmol). The resulting solution was added to the diazoketone solution in one portion (via syringe). The solution was allowed to warm to room temperature overnight. The solution was transferred to a 1-L, round-bottomed flask and the reaction vessel was rinsed with ethyl acetate (2×10 mL). The solution was evaporated and the residue was treated with saturated aqueous sodium bicarbonate (NaHCO₃) solution (100 mL) and stirred for 1 h at 23° C. Extraction with ethyl acetate (200 mL) gave the desired crude compound which was directly used in the next step.

### Step e): Tert-Butyl 3-[3-[2-[2-(cyclopropanecarbonyl)hydrazino]-2-oxo-ethyl]-1-bicyclo[1.1.1]pentanyl]azetidine-1-carboxylate

A solution of 2-[3-(1-tert-butoxycarbonylazetidin-3-yl)-1-bicyclo[1.1.1]pentanyl]acetic acid (1700.0 mg, 6.04 mmol) in DCM (50 mL) was treated with CDI (1273.68 mg, 7.85 mmol), at 23° C. The mixture was stirred for 45 min at this temperature, before being treated with cyclopropane carbohydrazide (665.45 mg, 6.65 mmol). The mixture was stored for another 18 h at 23° C., before being diluted with DCM (50 mL) and washed with DCM (50 mL). The organic layer was washed with water and brined, dried over Na2SO4, filtered and evaporated, to give the title compound (2.150 g, 89.09% yield) as a light yellow solid. MS (ESI): m/z=362.0 [M−H]⁻

### Step f): Tert-Butyl 3-[3-[(5-cyclopropyl-1,3,4-oxadiazol-1 yl)methyl]-1-bicyclo[1.1.1]pentanyl]azetidine-1-carboxylate

A solution of tert-butyl 3-[3-[2-[2-(cyclopropanecarbonyl)hydrazino]-2-oxo-ethyl]-1-bicyclo[1.1.1]pentanyl]azetidine-1-carboxylate (2150.0 mg, 5.92 mmol) in ACN (150 mL) was treated with with N,N-diisopropylethylamine (3.09 mL, 17.75 mmol) and p-toluenesulfonyl chloride (1466.13 mg, 7.69 mmol), at 23° C. The mixture was stirred for 12 h at this temperature, and for 24 h at 50° C., before being evaporated. Purification by FC (SiO₂; CHCl₃/ACN) gave the title compound (550.0 mg, 25.57% yield) as a light yellow oil. MS (ESI): m/z=290.2 [M-t-Bu+H]⁺

### Example M.13

### 2-[[3-(azetidin-3-yl)-1-bicyclo[1.1.1]pentanyl]methoxy]-5-(trifluoromethyl)pyridine; 2,2,2-trifluoroacetic Acid

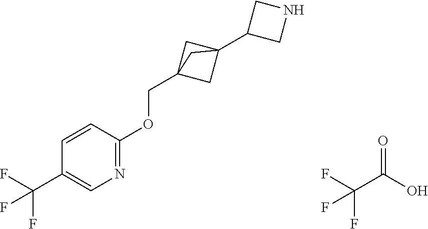 [SMILES: FC(F)(F)c1ccc(OCC23CC(C4CNC4)(C2)C3)nc1 ; O=C(O)C(F)(F)F]

A solution of 3-[3-[[5-(trifluoromethyl)-2-pyridyl]oxymethyl]-1-bicyclo[1.1.1]pentanyl]azetidine-1-carboxylic acid tert-butyl ester (300 mg, 0.753 mmol) in dichloromethane (3 mL) was treated with TFA (580.1 μL, 7.53 mmol), at 23° C. The mixture was stirred for 18 h at this temperature, before being evaporated to give the title compound (615 mg, quant.). MS (ESI): m/z=299.2 [M+H]⁺

### Step a): Tert-Butyl 3-[3-(hydroxymethyl)-1-bicyclo[1.1.1]pentanyl]azetidine-1-carboxylate

A solution of 3-(1-tert-butoxycarbonylazetidin-3-yl)bicyclo[1.1.1]pentane-1-carboxylic acid (CAS RN: 2227205-20-9; 6.0 g, 22.45 mmol) in THE (70 mL) was treated with borane-methyl sulfide complex (4262.87 mg, 56.11 mmol), at 0° C. The mixture was then stirred to reflux for 6 h, before being cooled down to 0° C., treated dropwise with MeOH (5 mL), and evaporated. The residue was dissolved in brine and EtOAc, and the aqueous layer was washed with EtOAc. The organic layers were combined, dried over Na₂SO₄, filtered, and evaporated, to give the title compound (5.4 g, 90.22% yield) as a white solid. MS (ESI): m/z=198.0 [M-boc+H]⁺

### Step b): 3-[3-(methylsulfonyloxymethyl)-1-bicyclo[1.1.1]pentanyl]azetidine-1-carbolic Acid Tert-Butyl Ester

A solution of 3-(3-methylol-1-bicyclo[1.1.1]pentanyl)azetidine-1-carboxylic acid tert-butyl ester (3.31 g, 13.07 mmol) in dichloromethane (45 mL) was treated with TEA (2.73 mL, 19.6 mmol) and Mesyl-Cl (1.12 mL, 14.37 mmol), at 0° C. The mixture was stirred for 10 min at this temperature, and 18 h at 23° C., before being diluted with dichloromethane and water. The aqueous phase was washed with dichloromethane, and the combined organic layers were dried over Na₂SO₄, filtered, and evaporated, to give the crude title compound (4.195 g, quart.) as a brown solid. MS (ESI): m/z=276.2 [M-tBu+H]⁺

### Step c): 3-[3-[[5-(trifluoromethyl)-2-pyridyl]oxymethyl]-1-bicyclo[1.1.1]pentanyl]azetidine-1-carboxylic Acid Tert-Butyl Ester

A solution of 5-(trifluoromethyl)-2-pyridone (CAS RN: 33252-63-0; 460.12 mg, 2.82 mmol) in N,N-dimethylformamide (11 mL) was treated with NaH (112.84 mg, 2.82 mmol), at 0° C. The mixture was stirred for 15 min at this temperature, before being treated 3-[3-(methylsulfonyloxymethyl)-1-bicyclo[1.1.1]pentanyl]azetidine-1-carboxylic acid tert-butyl ester (850 mg, 2.56 mmol). The mixture was stirred for 18 h at 90° C., before being treated with a few drops of saturated aqueous NH₄Cl. The mixture was concentrated, and the residue was dissolved in EtOAc and water. The aqueous phase was washed with EtOAc, and the combined organic layers were dried over Na₂SO₄, filtered, and evaporated. FC (SiO₂; heptane/EtOAc) gave the title compound (300 mg, 28% yield). MS (ESI): m/z=343.2 [M+H]⁺

In analogy to Example M. 13, the following building block was generated using the relevant commercial building block in Step c).

| | | | Building | MS, ESI: |
|---|---|---|---|---|
| Ex. | Structure | Systematic Name | Blocks | m/z |
| M.14 | 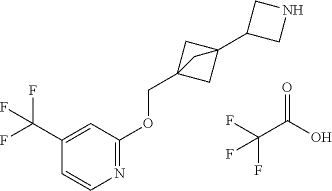 | 2-[[3-(azetidin-3-yl)- 1- bicyclo[1.1.1]pentanyl] methoxy]-4- (trifluoromethyl) pyridine; 2,2,2- trifluoroacetic acid | 2-Hydroxy-4- (trifluoro- methyl) pyridine (CAS RN: 50650-59-4) | 299.2 [M + H]⁺ |
| M.15 | 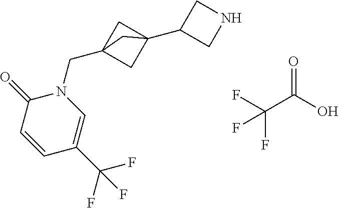 [SMILES: O=c1ccc(C(F)(F)F)cn1CC12CC(C3CNC3)(C1)C2 ; O=C(O)C(F)(F)F] | 1-[[3-(azetidin-3-yl)- 1- bicyclo[1.1.1]pentanyl] methyl]-5- (trifluoromethyl) pyridin-2-one; 2,2,2- trifluoroacetic acid | 5- (trifluoro- methyl)- 2-pyridone (CAS RN: 33252-63-0) | 299.2 [M + H]⁺ |
| M.16 | 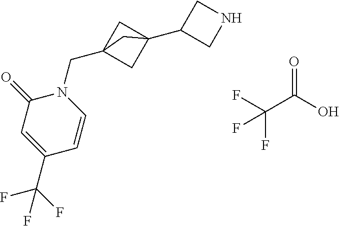 [SMILES: O=c1cc(C(F)(F)F)ccn1CC12CC(C3CNC3)(C1)C2 ; O=C(O)C(F)(F)F] | 1-[[3-(azetidin-3-yl)- 1- bicyclo[1.1.1]pentanyl] methyl]-4- (trifluoromethyl) pyridin-2-one; 2,2,2- trifluoroacetic acid | 2-Hydroxy-4- (trifluoro- methyl) pyridine (CAS RN: 50650-59-4) | 299.2 [M + H]⁺ |
| M.17 | 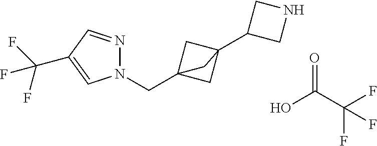 | 1-[[3-(azetidin-3-yl)- 1- bicyclo[1.1.1]pentanyl] methyl]-4- (trifluoromethyl) pyrazole; 2,2,2- trifluoroacetic acid | 4- Trifluoro- methyl- 1H-pyrazole (CAS RN: 52222-73-8) | 272.2 [M + H]⁺ |

### Example M.19

### 2-[[3-(azetidin-3-yl)-1-bicyclo[1.1.1]pentanyl]oxy]-5-(trifluoromethyl)pyrazine; 4-methylbenzenesulfonic Acid

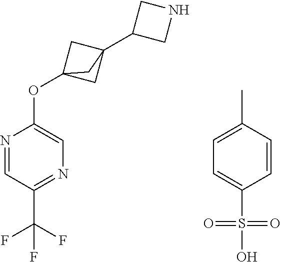 [SMILES: FC(F)(F)c1cnc(OC23CC(C4CNC4)(C2)C3)cn1 ; Cc1ccc(S(=O)(=O)O)cc1]

A solution of tert-butyl 3-[3-[5-(trifluoromethyl)pyrazin-2-yl]oxy-1-bicyclo[1.1.1]pentanyl]azetidine-1-carboxylate (200.0 mg, 0.52 mmol) in EtOAc (25 mL) was treated with PTSA (148.08 mg, 0.78 mmol), at 23° C. The mixture was stirred for 16 h at this temperature before being cooled down to 0° C. and stirred for 1 h at that temperature. The resulting precipitate was collected by filtration and washed twice with MTBE (2*50 mL), to give the title compound (190.0 mg, 76.03% yield) as a yellow solid. MS (ESI): m/z=286.2 [M+H]⁺

### Step a): Tert-Butyl 3-[3-[5-(trifluoro(methyl)pyrazin-2-yl]oxy-1-bicyclo[1.1.1]pentanyl]azetidine-1-carboxylate

A solution of tert-butyl 3-(3-hydroxy-1-bicyclo[1.1.1]pentanyl)azetidine-1-carboxylate (CAS RN: 2490406-07-8; 700.0 mg, 2.93 mmol) and 2-fluoro-5-(trifluoromethyl)pyrazine (CAS RN: 69045-82-5; 534.38 mg, 3.22 mmol) in ACN (30 mL) was treated with cesium carbonate (1906.1 mg, 5.85 mmol), at 25° C. under Ar. The mixture was stirred at 80° C. for 16 h, before being filtered. The cake was washed with ACN (30 mL), and the filtrate was evaporated. The residue was diluted with H₂O (30 mL) and extracted with EtOAc (3×10 mL). The combined organic layers were washed with saturated aqueous NaCl, dried over Na₂SO₄, and evaporated, to give the title compound (200 mg, 42.14% yield) as a light brown solid. MS (ESI): m/z=286 [M-boc+H]⁺

### Example M.21

### N-[3-(azetidin-3-yl)-1-bicyclo[1.1.1]pentanyl]-5-(trifluoromethyl)pyrazin-2-amine; 2,2,2-trifluoroacetic Acid

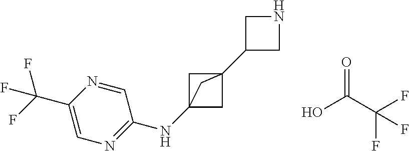 [SMILES: FC(F)(F)c1cnc(NC23CC(C4CNC4)(C2)C3)cn1 ; O=C(O)C(F)(F)F]

A solution of 3-[3-[[5-(trifluoromethyl)pyrazin-2-yl]amino]-1-bicyclo[1.1.1]pentanyl]azetidine-1-carboxylic acid tert-butyl ester (245 mg, 0.625 mmol) in dichloromethane (3 mL) was treated with TFA (481.2 uL, 6.25 mmol), at 23° C. The mixture was stirred for 18 h at this temperature before being evaporated, to give the crude title compound (493 mg, 99%). MS (ESI): m/z=285.1 [M+H]⁺

### Step a): Tert-Butyl 3-[3-(benzyloxycarbonylamino)-1-bicyclo[1.1.1]pentanyl]azetidine-1-carboxylate

To a solution of 3-(1-tert-butoxycarbonylazetidin-3-yl)bicyclo[1.1.1]pentane-1-carboxylic acid (CAS RN: 4.25 g, 15.9 mmol) and benzyl alcohol (3.29 mL, 31.8 mmol) in Toluene (60 mL) was added triethylamine (6.65 mL, 47.7 mmol), at 23° C. The mixture was stirred for 5 min at the temperature, and diphenyiphosphonic azide (3.6 mL, 16.69 mmol) was added. The mixture was stirred for another 15 min at 23° C., and for 16 h at 100° C. After cooling, it was poured into iced H₂O (100 mL) and extracted with MTBE. The organic layer was washed with H₂O and brine, dried over Na₂SO₄, filtered, and evaporated. Purification by FC gave the title compound (3.7 g, 59.36% yield) as a white solid. MS (ESI): m/z=371.2 [M−H]⁻

### Step b): Tert-Butyl 3-(3-amino-1-bicyclo[1.1.1]pentanyl)azetidine-1-carboxylate

To a solution of tert-butyl 3-[3-(benzyloxycarbonylamino)-1-bicyclo[1.1.1]pentanyl]azetidine-1-carboxylate (4.15 g, 11.14 mmol) in methanol (50 mL) was added palladium on carbon (10%) (0.58 mL, 0.56 mmol). The mixture was stirred 48 h at 23° C. under hydrogen atmosphere. The solids were removed by filtration and the filtrate was evaporated. The residue was treated with 4 M HCl in dioxane and stirred for 10 min at 0° C. The precipitate was collected by filtration, to give the title compound (2.6 g, 93.01% yield) as a colorless oil. MS (ESI): m/z=239.2 [M+H]⁺

### Step c): 3-[3-[[5-(trifluoromethyl)pyrazin-2-yl]amino]-1-bicyclo[1.1.1]pentanyl]azetidine-1-carboxylic Acid Tert-Butyl Ester

To a solution of 3-(3-amino-1-bicyclo[1.1.1]pentanyl)azetidine-1-carboxylic acid tert-butyl ester (150 mg, 0.629 mmol) and DIPEA (219.64 uL, 1.26 mmol) in N,N-dimethylformamide, extra dry (3 mL) cooled down to 0° C. was added followed by addition of 2-fluoro-5-(trifluoromethyl)pyrazine (146.34 mg, 0.881 mmol) after which the reaction mixture was stirred at 0° C. for 10 min and allowed to warm up to r.t after which the reaction was stirred for 3 h. Volatiles were evaporated, and purification by FC (SiO₂; heptane/EtOAc) gave the title compound (196 mg, 79%) as a white solid. MS (ES): m/z=329.2 [M-tBu+H]⁺

### Example M.22

### 2-[[3-(azetidin-3-yl)-1-bicyclo[1.1.1]pentanyl]sulfonyl]-5-(trifluoromethyl)pyridine; 4-methylbenzenesulfonic Acid

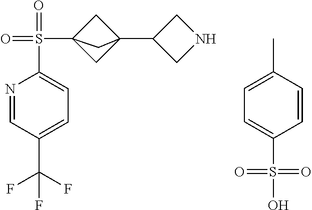 [SMILES: O=S(=O)(c1ccc(C(F)(F)F)cn1)C12CC(C3CNC3)(C1)C2 ; Cc1ccc(S(=O)(=O)O)cc1]

A solution of tert-butyl 3-[3-[[5-(trifluoromethyl)-2-pyridyl]sulfonyl]-1-bicyclo[1.1.1]pentanyl]azetidine-1-carboxylate (95.0 mg, 0.16 mmol) and PTSA (0.07 g, 0.38 mmol) in EtOAc (4 mL) was stirred at 25° C. for 24 h, before being evaporated. Crystallization from MTBE gave the title compound (79.8 mg, 96.0% yield) as a light brown solid. MS (ESI): m/z=333.2 [M+H]⁺

### Step a): 5-(trifluoromethyl)-2-[[5-(trifluoromethyl)-2-pyridyl]disulfanyl]pyridine

A solution of 2-mercapto-5-(trifluoromethyl)pyridine (CAS RN: 76041-72-0; 4.0 g, 22.33 mmol) in dichloromethane (250 mL) was treated with sodium hydrogen carbonate (2.44 g, 29.02 mmol), at 25° C. under Ar. The mixture was stirred for 20 min at this temperature, before being treated with iodine-131 (8.50 g, 33.49 mmol). The mixture was stirred for another 30 min at 25° C., treated with saturated aqueous Na₂S₂O₃, and stirred for 15 min. The layers were separated, and the organic layer was washed with water, dried over Na₂SO₄, filtered, and evaporated to give the title compound (3.6 g, 43.9% yield) as a yellow solid. MS (ESI): m/z=357.0 [M+H]⁺

### Step b): Tert-Butyl 3-[3-[[5-(trifluoromethyl)-2-pyridyl]sulfanyl]-1-bicyclo[1.1.1]pentanyl]azetidine-1-carboxylate

A solution of 5-(trifluoromethyl)-2-[[5-(trifluoromethyl)-2-pyridyl]disulfanyl]pyridine (1.0 g, 2.81 mmol) and 4-methylmorpholine (0.11 mL, 0.98 mmol) DCM (10 mL) was cooled to −30° C. under Ar, and treated dropwise with isobutyl chloroformate (0.13 mL, 0.98 mmol). The mixture was stirred for 15 min at −10° C., before being wrapped with aluminium foil to avoid light. 3-(1-Tert-butoxycarbonylazetidin-3-yl)bicyclo[1.1.1]pentane-1-carboxylic acid (0.25 g, 0.94 mmol) was added in one portion, and the mixture was stirred for 2 h at 0° C. before being filtered and evaporated (water bath below 40° C.). The resulting Barton ester was dissolved in benzene (20 mL) and 2-mercaptopyridine N-oxide sodium salt (0.15 g, 1.03 mmol) was added. Argon was bubbled through the solution for 10 min, and then the mixture was irradiated with a 500 W halogen lamp under argon atmosphere for 3 h. The solvent was evaporated. Purification by FC (SiO₂; hexane/MTBE) gave the title compound (100.0 mg, 22.7% yield) as a colorless oil. MS (ESI): m/z=401.2 [M+H]⁺

### Step c): Tert-Butyl 3-[3-[[5-(trifluoromethyl)-2-pyridyl]sulfonyl]-1-bicyclo[1.1.1]pentanyl]azetidine-1-carboxylate

3-Chloroperoxybenzoic acid (0.14 g, 0.75 mmol) was added to a solution of tert-butyl 3-[3-[[5-(trifluoromethyl)-2-pyridyl]sulfanyl]-1-bicyclo[1.1.1]pentanyl]azetidine-1-carboxylate (100.0 mg, 0.25 mmol) in DCM (10 mL) at 0° C. The reaction mixture was stirred at 23° C. for 12 h. The mixture was washed with aqueous Na₂S₂O₃ and NaHCO₃ solutions and water. The organic layer was dried (Na₂SO₄) and evaporated to give the title compound (95 mg, 66% yield) as a light yellow solid. MS (ESI): m/z=377.0 [M-tBu+H]⁺

In analogy to Example M.22, the following building block was generated using the relevant commercial building block in Step a).

| | | | Building | MS, ESI: |
|---|---|---|---|---|
| Ex. | Structure | Systematic Name | Blocks | m/z |
| M.34 | 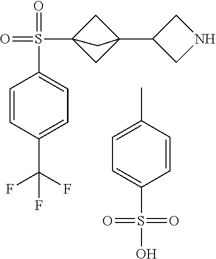 | 4- methylbenzenesulfonic acid; 3-[3-[4- (trifluoromethyl) phenyl]sulfonyl-1- bicyclo[1.1.1]pentanyl] azetidine | 4- (Trifluoro- methyl) benzenethiol (CAS RN: 825-83-2) | 332.0 [M + H]⁺ |

### Example M.25

### 1-[3-(azetidin-3-yl)-1-bicyclo[1.1.1]pentanyl]-3,5-dimethyl-pyrazole; 4-methylbenzenesulfonic Acid

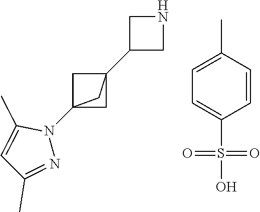

A solution of tert-butyl 3-[3-(3,5-dimethylpyrazol-1-yl)-1-bicyclo[1.1.1]pentanyl]azetidine-1-carboxylate (597.0 mg, 1.88 mmol) in MeOH (3 mL) was treated with PTSA (485.79 mg, 2.82 mmol), at 23° C. The mixture was stirred for 72 h at 40° C., before being evaporated. Trituration with ACN and purification by RP-HPLC gave the title compound (374.7 mg, 48.59% yield) as a light yellow solid. MS (ESI): m/z=218.2 [M+H]⁺

### Step a): Tert-Butyl 3-[3-(benzyloxycarbonylamino)-1-bicyclo[1.1.1]pentanyl]azetidine-1-carboxylate

A solution of 3-(1-tert-butoxycarbonylazetidin-3-yl)bicyclo[1.1.1]pentane-1-carboxylic acid (CAS RN: 2227205-20-9; 4.25 g, 15.9 mmol) and benzyl alcohol (3.29 mL, 31.8 mmol) in Toluene (60 mL) was treated with triethylamine (6.65 mL, 47.7 mmol), at 23° C. The mixture was stirred for 5 min at this temperature, before being treated with diphenylphosphonic azide (3.6 mL, 16.69 mmol). The mixture was stirred for another 15 min at this temperature and for 16 h at 100° C., before being cooled down. The mixture was poured into iced H₂O (100 mL) and extracted with MTBE. The organic layer was washed with H₂O and brine, dried over Na₂SO₄, filtered, and evaporated. Purification by FC (SiO₂) gave the title compound (3.7 g, 59.36% yield) as a white solid. MS (ESI): m/z=371.2 [M−H]⁻

### Step b): Tert-Butyl 3-(3-amino-1-bicyclo[1.1.1]pentanyl)azetidine-1-carboxylate

A solution of tert-butyl 3-[3-(benzyloxycarbonylamino)-1-bicyclo[1.1.1]pentanyl]azetidine-1-carboxylate (4.15 g, 11.14 mmol) in MeOH (50 mL) was treated with Pd/C (10%) (0.58 mL, 0.56 mmol), under Ar. The mixture was stirred for 48 h at 23° C. under hydrogen atmosphere, before being filtered off. The filtrate was evaporated, to give the title compound (2.6 g, 93.01% yield) as a colorless oil. MS (ESI): m/z=239.2 [M+H]⁺

### Step c): Tert-Butyl 3-[3-(3,5-dimethylpyrazol-1 yl)-1-bicyclo[1.1.1]pentanyl]azetidine-1-carboxylate

A solution of tert-butyl 3-(3-amino-1-bicyclo[1.1.1]pentanyl)azetidine-1-carboxylate (1.3 g, 5.45 mmol) in DMF (10 mL) was treated with acetylacetone (0.61 mL, 6.0 mmol) and O-(4-nitrobenzoyl)hydroxylamine (1.49 g, 8.18 mmol), at 23° C. The mixture was stirred at 85° C. for 2 h, cooled down, and treated with water (25 mL). The mixture was extracted with EtOAc (3 times 15 mL each). The organic layers were dried over Na₂SO₄, filtered and evaporated. Purification by RP-HPLC gave the title compound (597.0 mg, 32.76% yield). MS (ESI): m/z=318.2 [M+H]⁺

### Example M.32

### 5-[3-(azetidin-3-yl)-1-bicyclo[1.1.1]pentanyl]-3-methyl-1H-pyrazole; 4-methylbenzenesulfonic Acid

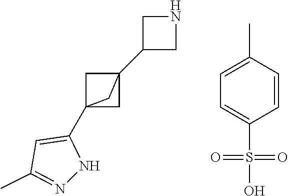

A solution of tert-butyl 3-[3-(3-methyl-1H-pyrazol-5-yl)-1-bicyclo[1.1.1]pentanyl]azetidine-1-carboxylate (360.0 mg, 1.19 mmol) and PTSA (473.98 mg, 2.49 mmol) in MeOH (15 mL) was stirred at 20° C. for 48 h, before being evaporated. Purification by RP-HPLC gave the title compound (268.9 mg, 57.34% yield) as a white solid. MS (ESI): m/z=204 [M+H]⁺

### Step a): Tert-Butyl 3-[3-(3-oxobutanoyl)-1-bicyclo[1.1.1]pentanyl]azetidine-1-carboxylate

Part 1. A solution of 3-(1-tert-butoxycarbonylazetidin-3-yl)bicyclo[1.1.1]pentane-1-carboxylic acid (1.5 g, 5.61 mmol) and 4-methylmorpholine (0.68 mL, 6.17 mmol) in THE (60 mL) was treated dropwise with isobutyl chloroformate (766.38 mg, 5.61 mmol), at −30° C. under Ar. The mixture was stirred for another hour at 0° C., and the resulting precipitate was filtered off. The filtrate—tert-butyl 3-(3-isobutoxycarbonyloxycarbonyl-1-bicyclo[1.1.1]pentanyl)azetidine-1-carboxylate (as solution in THF)—was used directly to the next step.

Part 2. A solution of diisopropylamine (1.03 mL, 7.29 mmol) in THF (30 mL) was treated with n-Butyllithium 23% solution in hexane (503.16 mg, 7.86 mmol), at −78° C. under Ar. The mixture was stirred for 10 min at −40° C.

Part 3. Acetone Li-salt. Acetone (0.54 mL, 7.29 mmol) was added to freshly prepared lithium diisopropyl amide (7.3 mmol) (THF/hexane solution from Part 2). at −30° C. and the obtained solution was stirred for 20 min at 0° C.

Part 4. The product from Part 1 was added to the product from Part 3 (Acetone Li-salt) at 0° C. The mixture was allowed to warm up to 23° C. and stirred for 18 h at that temperature, before being diluted with saturated ammonium chloride solution (30 mL) and citric acid (30 mL, water solution) and extracted with TBME (3×70 mL). The combined organic layers were washed with water, dried over sodium sulfate and evaporated. Purification by FC (SiO₂; hexane/MTBE) gave the title compound (380.0 mg, 20.93% yield) as a mixture of tautomers.

### Step 2): Tert-Butyl 3-[3-(3-methyl-1H-pyrazol-5-yl)-1-bicyclo[1.1.1]pentanyl]azetidine-1-carboxylate

A solution of tert-butyl 3-[3-(3-oxobutanoyl)-1-bicyclo[1.1.1]pentanyl]azetidine-1-carboxylate (380.0 mg, 1.24 mmol) in Methanol (30 mL) was treated with hydrazine hydrate (61.89 mg, 1.24 mmol), at 23° C. The mixture was stirred at 60° C. for 4 h and evaporated. The residue was partitioned between water and TBME, the organic layer was dried over Na₂SO₄, filtered and evaporated to give the title compound (360.0 mg, 91.18% yield) as a white semisolid. MS (ESI): m/z=304.2 [M+H]⁺

In analogy to Example M.32, the following building block was generated using the relevant commercial building block in Step a).

| | | | Building | MS, ESI: |
|---|---|---|---|---|
| Ex. | Structure | Systematic Name | Blocks | m/z |
| M.33 | 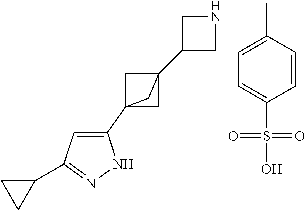 | 5-[3-(azetidin-3-y])-1- bicyclo[1.1.1]pentanyl]- 3-cyclopropyl-1H- pyrazole; 4- methylbenzenesulfonic acid | cyclopropyl methyl ketone (CAS RN: 765-43-5) | 230.2 [M + H]⁺ |

### Example M.35

### 4-methylbenzenesulfonic acid; 3-[3-[[1-(trifluoromethyl)cyclopropyl]methylsulfonyl]-1-bicyclo[1.1.1]pentanyl]azetidine

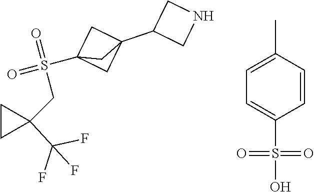 [SMILES: O=S(=O)(CC1(C(F)(F)F)CC1)C12CC(C3CNC3)(C1)C2 ; Cc1ccc(S(=O)(=O)O)cc1]

A solution of tert-butyl 3-[3-[[1-(trifluoromethyl)cyclopropyl]methylsulfonyl]-1-bicyclo[1.1.1]pentanyl]azetidine-1-carboxylate (155.0 mg, 0.38 mmol) and PTSA (108.01 mg, 0.57 mmol) in MeOH (2 mL) was stirred at 25° C. for 18 h, before being evaporated. The residue was triturated with MTBE (15 mL), to give the title compound (186.5 mg, 97.19% yield) as a white solid. MS (ESI): m/z=310.1 [M+H]⁺

### Step a): Tert-Butyl 3-[3-(2 pyridylsulfanyl)-1-bicyclo[1.1.1]pentanyl]azetidine-1-carboxylate

A solution of 2,2′-dipyridyl disulfide (12.36 g, 56.11 mmol) and 4-methylmorpholine (2.16 mL, 19.64 mmol) in DCM (120 mL) was treated dropwise with isobutyl chloroformate (2.56 mL, 19.64 mmol), at −30° C. under Ar. The mixture was stirred for 15 min at −15° C. The reaction vessel was shielded from light (i.e. wrapped with aluminum foil), and 3-(1-tert-butoxycarbonylazetidin-3-yl)bicyclo[1.1.1]pentane-1-carboxylic acid (5.0 g, 18.7 mmol) was added in one portion. After stirring at −5° C. for 2 h, the mixture was filtered and evaporated to dryness under reduced pressure (bath temperature less than 40° C.). The resulting Barton ester was dissolved in Benzene (240 mL) and 2-mercaptopyridine N-oxide sodium salt (3.07 g, 20.57 mmol) was added. Argon was bubbled through the solution for 10 min, and the mixture was irradiated with a 500 W halogen lamp under argon atmosphere for 3 h. The solvent was evaporated. Purification by FC (SiO₂; PE/MTBE) gave the title compound (780.0 mg, 11.16% yield) as a white solid. MS (ESI): m/z=333.2 [M+H]⁺

### Step b): Tert-Butyl 3-[3-(2-pyridylsulfonyl)-1-bicyclo[1.1.1]pentanyl]azetidine-1-carboxylate

A solution of tert-butyl 3-[3-(2-pyridylsulfanyl)-1-bicyclo[1.1.1]pentanyl]azetidine-1-carboxylate (2.3 g, 6.92 mmol) in DCM (150 mL) was treated portionwise with 3-chloroperoxybenzoic acid (3581.58 mg, 20.75 mmol), at 0° C. The mixture was stirred for 18 h at 23° C., before being cooled down again to 0° C. The resulting precipitate was filtered off and washed with DCM (50 mL). The filtrate was washed with saturated aqueous Na₂S₂O₃ (20 mL) and saturated aqueous NaHCO₃ (20 mL). The combined organic layers were dried over Na₂SO₄, filtered and evaporated. Purification by FC (SiO₂; CHCl₃/ACN) gave the title compound (1.5 g, 56.52% yield) as a white solid. MS (ESI): m/z=365.2 [M+H]⁺

### Step c): Sodium; 3-(1-tert-butoxycarbonylazetidin-3 yl)bicyclo[1.1.1]pentane-1 sulfinate

A suspension of sodium hydride, 60% in oil (290 mg, 7.1 mmol) in THE (36 mL) was treated dropwise with ethanethiol (0.93 mL, 12.48 mmol), at 0° C. under Ar. The mixture was stirred for 2 h at 23° C., before being treated with a solution of tert-butyl 3-[3-(2-pyridylsulfonyl)-1-bicyclo[1.1.1]pentanyl]azetidine-1-carboxylate (1300.0 mg, 3.57 mmol) in THE (20 mL). The mixture was stirred for another 18 h at 23° C., before being diluted with Et₂O (250 mL). The resulting precipitate was filtered, washed with Et₂O (50 mL), and dried, to give the crude title compound (1.35 g, 97.87% yield). MS (ESI): m/z=286.2 [M+H-tBu]⁺

### Step d): Tert-Butyl 3-[3-[[1-(trifluoromethyl)cyclopropyl]methylsulfonyl]-1-bicyclo[1.1.1]pentanyl]azetidine-1-carboxylate

A suspension of sodium; 3-(1-tert-butoxycarbonylazetidin-3-yl)bicyclo[1.1.1]pentane-1-sulfinate (300.0 mg, 0.97 mmol) (80% purity) in DMF (6 mL) was treated with 1-(bromomethyl)-1-(trifluoromethyl)cyclopropane (295.29 mg, 1.45 mmol), at 23° C. The mixture was stirred at 44° C. for 18 h, before being diluted with water (30 mL) and extracted with MTBE (3×25 mL). The combined organic layers were dried over Na₂SO₄, filtered and evaporated. Purification by RP-HPLC gave the title compound (130.0 mg, 32.08% yield) as a white solid. MS (ES): m/z=310.2 [M-Boc+H]⁺

### Example M.36

### N-[[5-(azetidin-3-yl)pyrazin-2-yl]methyl]-1-(trifluoromethyl)cyclopropanamine; 2,2,2-trifluoroacetic Acid

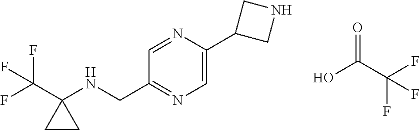 [SMILES: FC(F)(F)C1(NCc2cnc(C3CNC3)cn2)CC1 ; O=C(O)C(F)(F)F]

A solution of tert-butyl 3-[5-[[[1-(trifluoromethyl)cyclopropyl]amino]methyl]pyrazin-2-yl]azetidine-1-carboxylate (20.0 mg, 0.05 mmol) in DCM (0.5 mL) was treated with TFA (0.1 mL) stinted at 20° C. for 1 h before being evaporated, to give the title compound (10.0 mg, 68.39% yield) as a yellow oil. MS (ESI): m/z=273.0 [M+H]⁺

### Step a): (E)-1-(5-chloropyrazin-2 yl)-N-[1-(trifluoromethyl)cyclopropyl]methanimine

A solution of tert-butyl 5-chloropyrazine-2-carbaldehyde (3.0 g, 21.05 mmol) in DCM (40 mL) was treated with 1-(trifluoromethyl)cyclopropanamine hydrochloride (3.4 g, 21.05 mmol) and triethylamine (2.93 mL, 21.05 mmol), at 23° C. The mixture was stirred for 18 h at this temperature, before being diluted with water. The organic layer was dried over Na₂SO₄, filtered, and evaporated, to give the title compound (5.10 g. 87.37% yield) which was directly used in the next step.

### Step b): N [(5-chloropyrazin-2 yl)methyl]-1-(trifluoromethyl)cyclopropanamine

A solution of (E)-1-(5-chloropyrazin-2-yl)-N-[1-(trifluoromethyl)cyclopropyl]methanimine (5.1 g, 20.4 mmol) in MeOH (100 mL) was treated with sodium borohydride (1.93 g, 51.08 mmol), at 0° C. The mixture was stirred for 18 h at 23° C., before being evaporated. The residue was diluted with water and extracted with EtOAc. The organic layer was washed with NaHSO₄ solution, dried over Na₂SO₄, filtered and evaporated, to give the title compound (4.0 g, 54.46% yield) which was directly used in the next step. MS (ESI): m/z=251.9 [M+H]⁺

### Step c): Tert-Butyl 3-[5-[[[1-(trifluoromethyl)cyclopropyl]amino]methyl]pyrazin-2-yl]azetidine-1-carboxylate

The reaction was set up with three batches in parallel. A solution of Zinc (389.72 mg, 5.96 mmol) in DMA (8 mL) was treated with 1,2-dibromoethane (74.65 mg, 0.4 mmol) and chlorotrimethylsilane (43.17 mg, 0.4 mmol). The mixture was heated to 60° C. and stirred for 15 min, before being treated with a solution of 1-Boc-3-iodoazetidine (1237.57 mg, 4.37 mmol) in DMA (8 mL). The mixture was stirred for another 15 min, before being cooled down to 20° C. and treated with N-[(5-chloropyrazin-2-yl)methyl]-1-(trifluoromethyl)cyclopropanamine (1.0 g, 3.97 mmol), 1,1-bis(diphenylphosphino)feirocene-palladium(II)dichloridechloromethane complex (162.26 mg, 0.2 mmol) and CuI (0.01 mL, 0.2 mmol). The mixture was heated to 80° C. and stinted for 12 h. The residue was extracted with ethyl acetate (150 mL×3). The combined organic layers were washed with brine (150 mL×3), dried with anhydrous Na₂SO₄. filtered and evaporated. Purification by FC (SiO₂; PE/EtOAc) gave the title compound (1.4 g, 47.3% yield) as a yellow solid. MS (ESI): m/z=317.0 [M+H]⁺

### Example M.37

### 4-methylbenzenesulfonic Acid; 3-[4-[1-(trifluoromethylsulfonyl)cyclopropyl]phenyl]azetidine

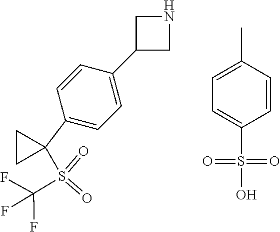 [SMILES: O=S(=O)(C(F)(F)F)C1(c2ccc(C3CNC3)cc2)CC1 ; Cc1ccc(S(=O)(=O)O)cc1]

A solution of tert-butyl 3-[4-[1-(trifluoromethylsulfonyl)cyclopropyl]phenyl]azetidine-1-carboxylate (700.0 mg, 1.73 mmol) and PTSA (356.78 mg, 2.07 mmol) in EtOAc (7 mL) was stirred at 80° C. for 12 h, before being evaporated. The residue was suspended in deionized water (30 mL) and lyophilized, to give the title compound (753.0 mg, 88.7% yield) as an off-white solid. MS (ESI): m/z=306.1 [M+H]⁺

### Step a): 1-bromo-4-(trifluoromethylsulfonylmethyl)benzene

A solution of 4-bromobenzyl bromide (CAS RN: 589-15-1; 5.0 g, 20.01 mmol) in DMF (30 mL) was treated with sodium trifluoromethanesulfinate (9.37 g, 60.02 mmol) and potassium iodide (0.83 g, 5.0 mmol), at 23° C. The mixture was stirred for 1 h at 120° C., before being cooled down and treated with water. The aqueous solution was adjusted to pH 2 with 3 M aqueous HCl, and evaporated, to give the crude title compound (5.43 g, 89.55% yield) as a light yellow solid. ¹H NMR (400 MHz, CHLOROFORM-d) δ=7.64-7.56 (m, 2H), 7.30 (d, J=8.4 Hz, 2H), 4.44 ppm (s, 2H).

### Step b): 1-bromo-4-[1-(trifluoromethylsulfonyl)cyclopropyl]benzene

A solution of 1-bromo-4-(trifluoromethylsulfonylmethyl)benzene (4.9 g, 16.17 mmol) in DMF (50 mL) was treated with 1,2-dibromoethane (3.04 g, 16.17 mmol) and K₂CO₃ (6.69 g, 48.5 mmol), at 23° C. The mixture was stirred for 12 h at 110° C., before being cooled down and poured into water (100 mL). The aqueous layer was extracted with ethyl acetate (3×30 mL). The combined organic layers were dried over Na₂SO₄, filtered and evaporated. Purification by FC (SiO₂; PE/EtOAc) gave the title compound (3.69 g, 69.35% yield) as a yellow oil. ¹H NMR (400 MHz, CHLOROFORM-d) δ=7.59-7.49 (m, 2H), 7.41 (d, J=8.4 Hz, 2H), 7.27 (s, 1H), 2.07-2.05 (m, 2H), 1.54-1.46 (m, 2H), 1.27 (t, J=7.2 Hz, 1H), 1.31-1.22 ppm (m, 1H).

### Step c): Tert-Butyl 3-[4-[1-(trifluoromethylsulfonyl)cyclopropyl]phenyl]azetidine-1-carboxylate

A solution of 1-bromo-4-[1-(trifluoromethylsulfonyl)cyclopropyl]benzene (1.0 g, 3.04 mmol) in DME (10 mL) was treated with 1-Boc-3-iodoazetidine (1.72 g, 6.08 mmol), Ir[dF(CF₃)ppy]₂(dtbpy)(PF₆) (34.03 mg, 0.03 mmol), NiCl₂·dtbbpy (6.05 mg, 0.02 mmol), TTMSS (755.48 mg, 3.04 mmol), and Na₂CO₃ (644.12 mg, 6.08 mmol) and the reaction was stirred at 2.5° C. and irradiated with a 34 W blue LED lamp (7 cm away), with cooling fan to keep the reaction temperature at 25° C. stirred for 14 h and under N₂. The mixture was evaporated. Purification by RP-HPLC gave the title compound (487.0 mg, 39.53% yield) as a yellow oil. MS (ESI): m/z=350.2 [M-C₄H⁸+H]⁺

### Example M.38

### 1-[4-(azetidin-3-yl)phenyl]-3-cyclopropyl-1,2,4-triazole; 4-methylbenzenesulfonic Acid

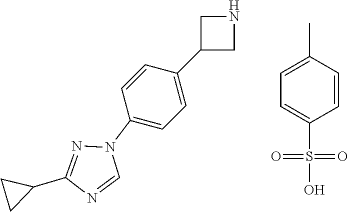 [SMILES: c1cc(-n2cnc(C3CC3)n2)ccc1C1CNC1 ; Cc1ccc(S(=O)(=O)O)cc1]

A solution of 3-[4-(3-cyclopropyl-1,2,4-triazol-1-yl)phenyl]azetidine-1-carboxylic acid tert-butyl ester (81.5 mg, 0.239 mmol) in EtOAc (0.405 mL) was treated with a solution of PTSA (93.36 mg, 0.491 mmol) in EtOAc (0.405 mL), at 23° C. The mixture was stirred at reflux for 2 h, before being cooled down and evaporated, affording the title compound (0.106 g; 68.6%) as a light yellow solid. MS (ESI): m/z=241.2 [M+H]⁺

### Step a): 1-(4-bromophenyl)-3-cyclopropyl-1,2,4-triazole

A solution of 3-cyclopropyl-1H-1,2,4-triazole (CAS RN: 1211390-33-8; 109.13 mg, 1 mmol) and 4-bromophenylboronic acid (CAS RN: 5467-74-3; 301.25 mg, 1.5 mmol) in dichloromethane (9.94 mL) was treated with copper (II) acetate (245.2 mg, 1.35 mmol), at 23° C. under air. The mixture was stirred for another 72 h at this temperature, before being filtered. The cake was washed with DCM, and the filtrate was evaporated. Purification by FC (SiO₂; DCM/MeOH) gave the title compound (0.053 g, 19.8%) as a way solid. MS (ESI): m/z=264.1 [M+H]⁺

### Step b): 3-[4-(3-cyclopropyl-1,2,4-triazol-1 yl)phenyl]azetidine-1-carboxylic Acid Tert-Butyl Ester

To a 20 mL vial equipped with a stir bar was added photocatalyst (IR[DF(CF3)PPY]2(DTBPY))PF6 (4.84 mg. 0.004 mmol), 1-(4-bromophenyl)-3-cyclopropyl-1,2,4-triazole (114 mg, 0.432 mmol), tert-butyl 3-bromoazetidine-1-carboxylate (105.94 μL, 0.647 mmol), tris(trimethylsilyl)silane (133.16 μL, 0.432 mmol) and anhydrous sodium carbonate (91.49 mg, 0.863 mmol). The vial was sealed and placed under argon before DME (3.67 mL) was added. To a separate vial was added Nickel(II) chlrodie ethylene glycol dimethyl ether complex (1.9 mg, 0.009 mmol) and 4,4′-di-tert-butyl-2,2′-bipyridine (2.32 mg, 0.009 mmol). The precatalyst vial was sealed, purged with argon then to it was added DME (1.47 mL). The precatalyst vial was sonicated for 5 min, after which, 0.37 mL (0.5 mol % catalyst, 0.005 eq) of it was syringed into the reaction vessel. The reaction mixture, a suspension, was degassed with argon. The reaction was stirred and irradiated with a 420 nm lamp for 16 h (75% intensity). The reaction was quenched by exposure to air, filtered and washed with a small volume of EtOAc. The filtrate was treated with silica gel and evaporated. Purification by FC (SiO₂; heptane/EtOAc) gave the title compound (0.082 g; 44.9%) as a light yellow oil. MS (ESI): m/z=341.3 [M+H]⁺

In analogy to Example M.38, the following building blocks were generated using the relevant commercial building block in Step a).

| | | | Building | MS, ESI: |
|---|---|---|---|---|
| Ex. | Structure | Systematic Name | Blocks | m/z |
| M.39 | 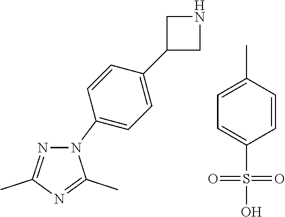 [SMILES: Cc1nc(C)n(-c2ccc(C3CNC3)cc2)n1 ; Cc1ccc(S(=O)(=O)O)cc1] | 1-[4-(azetidin-3- yl)phenyl]-3,5- dimethyl-1,2,4- triazole; 4- methylbenzenesulfonic acid | 3,5-dimethyl- 1H-1,2,4- triazole (CAS RN: 7343-34- 2) | 229.2 [M + H]⁺ |
| M.40 | 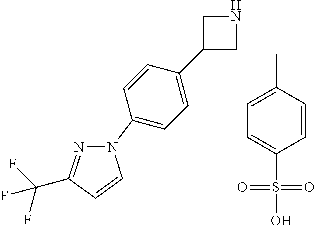 | 1-[4-(azetidin-3- yl)phenyl]-3- (trifluoromethyl) pyrazole; 4- methylbenzenesulfonic acid | 3- (trifluoro- methyl)- 1H-pyrazole (CAS RN: 20154-03-4) | 268.2 [M + H]⁺ |
| M.41 | 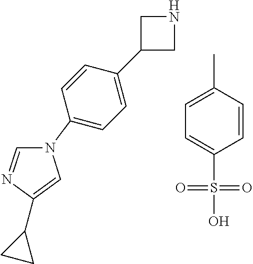 | 1-[4-(azetidin-3- yl)phenyl]-4- cyclopropyl- imidazole; 4- methylbenzenesulfonic acid | 4-cyclopropyl- 1H-imidazole (CAS RN: 89830-98-8) | 240.2 [M + H]⁺ |
| M.42 | 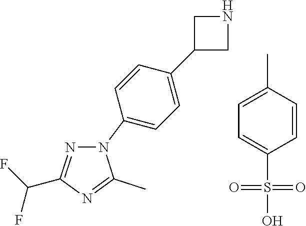 | 1-[4-(azetidin-3- yl)phenyl]-3- (difluoromethyl)-5- methyl-1,2,4- triazole; 4- methylbenzenesulfonic acid | 5- (difluoro- methyl)- 3-methyl- 1H-1,2,4- triazole (CAS RN: 1094760- 03-8) | 265.1 [M + H]⁺ |
| M.46 | 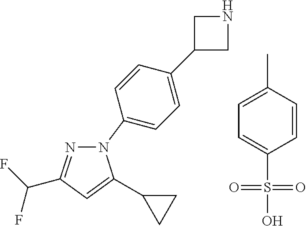 | 1-[4-(azetidin-3- yl)phenyl]-5- cyclopropyl-3- (difluoromethyl) pyrazole; 4- methylbenzenesulfonic acid | 5-cyclopropyl- 3- (difluoro- methyl)- 1H-pyrazole (CAS RN: 1062296-03-0) | 290.2 [M + H]⁺ |
| M.47 | 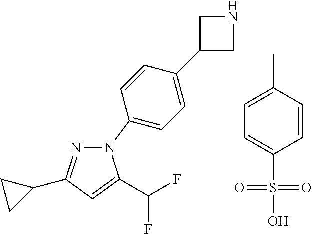 | 1-[4-(azetidin-3- yl)phenyl]-3- cyclopropyl-5- (difluoromethyl) pyrazole; 4- methylbenzenesulfonic acid | 5-cyclopropyl- 3- (difluoro- methyl)- 1H-pyrazole (CAS RN: 1062296-03-0) | 290.2 [M + H]⁺ |
| M.51 | 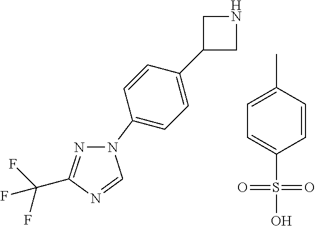 | 1-[4-(azetidin-3- yl)phenyl]-3- (trifluoromethyl)- 1,2,4-triazole; 4- methylbenzenesulfonic acid | 3- (trifluoro- methyl)- 1H-1,2,4- triazole (CAS RN: 60406-75- 9) | 269.2 [M + H]⁺ |

### Example M.43

### 5-[4-(azetidin-3-yl)phenyl]-3-(trifluoromethyl)-1H-pyrazole; 4-methylbenzenesulfonic Acid

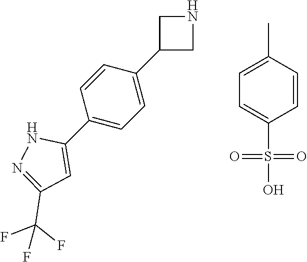 [SMILES: FC(F)(F)c1cc(-c2ccc(C3CNC3)cc2)[nH]n1 ; Cc1ccc(S(=O)(=O)O)cc1]

A solution of 3-[4-[3-(trifluoromethyl)-1H-pyrazol-5-yl]phenyl]azetidine-1-carboxylic acid tert-butyl ester (18.18 g, 49.49 mmol) in EtOAc (140 mL) was treated with PTSA (10.35 g, 54.44 mmol), at 23° C. The mixture was stirred for 45 min at 80° C., before being cooled down and filtered. The cake was washed with EtOAc and dried, to give the title compound (18.694 g, 85.9%) as a colorless solid. MS (ESI): m/z=268.2 [M+H]⁺

### Step a): 3-[4-[3-(trifluoromethyl)-1H-pyrazol-5-yl]phenyl]azetidine-1-carboxylic Acid Tert-Butyl Ester

In an argon filled flask were combined 3-bromoazetidine-1-carboxylic acid tert-butyl ester (CAS RN: 1064194-10-0; 10.71 g, 45.35 mmol), 5-(4-bromophenyl)-3-(trifluoromethyl)-1H-pyrazole (CAS RN: 219986-65-9; 12 g, 41.23 mmol), bis[3,5-difluoro-2-[5-(trifluoromethyl)-2-pyridyl]phenyl]iridium(1+); 4-tert-butyl-2-(4-tert-butyl-2-pyridyl)pyridine; hexafluorophosphate (462.53 mg, 412.27 umol), Na₂CO₃ (8.74 g, 82.45 mmol) and bis(trimethylsilyl)silyl-trimethyl-silane (12.72 mL, 41.23 mmol) and the mixture was suspended in ethylene glycol dimethyl ether (240 mL) and degassed with argon by bubbling it through the suspension. In a sealed and argon filled vial were combined dichloronickel; 1,2-dimethoxyethane (45.29 mg, 206.14 umol) and 4-tert-butyl-2-(4-tert-butyl-2-pyridyl)pyridine (55.33 mg, 206.14 umol) together with ethylene glycol dimethyl ether (24 mL) and this mixture was degassed for 5 minutes by bubbling argon through it. After being sonicated for 5 minutes, the precatalyst was syringed into the main reaction flask. After degassing with argon, the suspension was vigourously stirred under a blue LED lamp for 120 h. The suspension was filtered. To the filtrate was added silica gel (60 g) and the mixture was evaporated. Purification by FC (SiO₂; heptane/EtOAc) gave the title compound (8.17 g, 51.2%) as a light yellow solid. MS (ESI): m/z=366.3 [M−H]⁻

### Example M.44

### 4-[4-(azetidin-3-yl)phenyl]-1-methyl-3-(trifluoromethyl)pyrazole; 4-methylbenzenesulfonic Acid

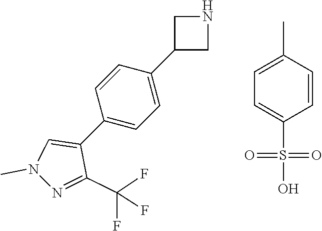 [SMILES: Cn1cc(-c2ccc(C3CNC3)cc2)c(C(F)(F)F)n1 ; Cc1ccc(S(=O)(=O)O)cc1]

A solution of 3-[4-[1-methyl-3-(trifluoromethyl)pyrazol-4-yl]phenyl]azetidine-1-carboxylic acid tert-butyl ester (133 mg, 0.349 mmol) in EtOAc (2.02 mL) was treated with PTSA (135.98 mg, 0.715 mmol), at 23° C. The mixture was stirred for 2 h at 80° C., before being cooled down and filtered out. The cake was dried, to give the title compound (0.227 g; 93.9%) as a yellow solid. MS (ESI): m/z=282.2 [M+H]⁺

### Step a): 3-[4-[1-methyl-3-(trifluoromethyl)pyrazol-4-yl]phenyl]azetidine-1-carboxylic Acid Tert-Butyl Ester

A solution of 1-methyl-4-(4,4,5,5-tetramethyl-1,3,2-dioxaborolan-2-yl)-3-(trifluoromethyl)pyrazole (CAS RN: 1218790-53-4; 106.11 mg, 0.384 mmol), 3-(4-bromophenyl)azetidine-1-carboxylic acid tert-butyl ester (CAS RN: 1064194-10-0; 120 mg, 0.384 mmol), potassium carbonate (265.61 mg, 1.92 mmol) and tetrakis(triphenylphosphine)palladium (0) (22.21 mg, 0.019 mmol) in THE (1.81 mL) and Water (0.181 mL) was vigorously stirred under argon at 80° C. for 3 h. The reaction mixture was poured on water (5 mL) and ethyl acetate (5 mL) and the layers were separated. The aqueous layer was extracted twice with ethyl acetate (2×5 mL). The organic layers were combined and dried over MgSO₄, filtered, treated with silica gel and evaporated. Purification by FC (SiO₂; heptane/EtOAc) gave the title compound (0.135 g, 87.4%) as a yellow gum. MS (ESI): m/z=326.2 [M-C₄H₈+H]⁺

### Example M.45

### 2-[3-(4-piperidyl)-5-(trifluoromethyl)indazol-1-yl]ethanol; Hydrochloride

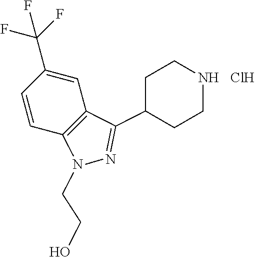 [SMILES: OCCn1nc(C2CCNCC2)c2cc(C(F)(F)F)ccc21 ; Cl]

A solution of tert-butyl 4-[1-(2-hydroxyethyl)-5-(trifluoromethyl)indazol-3-yl]piperidine-1-carboxylate (600.0 mg, 1.45 mmol) in HCl/dioxane (5.0 mL, 2.0 mmol) at 20° C. The mixture was stirred at 20° C. for 1 h, before being evaporated, to give the title compound (405.0 mg, 84.61% yield) as a yellow solid. MS (ESI): m/z=314.1 [M+H]⁺

### Step a): Tert-Butyl 4-[2 fluoro-5-(trifluoromethyl)benzoyl]piperidine-1-carboxylate

A solution of 3-bromo-4-fluorobenzotrifluoride (CAS RN: 68322-84-9; 5.35 g, 22.03 mmol) in THE (30 mL) was treated dropwise with n-BuLi (12.34 mL, 30.84 mmol), at −78° C. under Ar. The mixture was stirred for 30 min at this temperature, before being treated with 1-Boc-4-(methoxy-methyl-carbamoyl)-piperidine (4.0 g, 14.69 mmol). The mixture was allowed to warm up to 20° C. and stirred for 2 h at this temperature, before being treated with 10 mL of water and extracted with EtOAc. The combined organic layers were dried over Na₂SO₄, filtered, and evaporated. Purification by FC (SiO₂; PE/EtOAc) gave the title compound (800.0 mg, 19.35% yield) as an orange solid. MS (ESI): m/z=320.0 [M-C₄H₈+H]⁺

### Step b): Tert-Butyl 4-[1-(2-hydroxyethyl)-5-(trifluoromethyl)indazol-3 yl]piperidine-1-carboxylate

A solution of tert-butyl 4-(5-trifluoromethyl-2-fluoro-benzoyl)piperidine-1-carboxylate (800.0 mg, 2.34 mmol) in 1-butanol (15 mL) was treated with 2-hydrazineylethan-1-ol (391.85 mg, 5.15 mmol), at 20° C. The mixture was stirred at 120° C. for 12 h, before being evaporated. Purification by FC (SiO₂; PE/EtOAc) gave the title compound (680.0 mg, 76.48% yield) as a yellow solid. MS (ESI): m/z=324.1 [M-C₄H₅+H]⁺

### Example M.48

### (4-ethylphenyl)-piperazin-1-yl-methanone; Hydrochloride

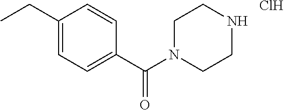 [SMILES: CCc1ccc(C(=O)N2CCNCC2)cc1 ; Cl]

A solution of tert-butyl 4-(4-ethylbenzoyl)piperazine-1-carboxylate (1730.0 mg, 5.43 mmol) in 4 M HCl/MeOH (20.0 mL, 5.43 mmol) was stirred at 25° C. for 12 h. The mixture was then evaporated, to give the title compound (1.19 g, quant.) as a white solid.

### Step a): Tert-Butyl 4-(4-ethylbenzoyl)piperazine-1-carboxylate

A solution of 4-ethylbenzoyl chloride (CAS RN: 16331-45-6; 1.0 g, 5.93 mmol) in DCM (1.5 mL) was treated with triethylamine (2.48 mL, 17.79 mmol) and 1-Boc-piperazine (CAS RN: 76535-74-5; 1.89 mL, 11.86 mmol), at 23° C. The mixture was stirred at 25° C. for 12 h and evaporated. Purification by RP-HPLC gave the title compound (1.73 g, 91.62% yield) as a white solid. MS (ESI): m/z=319.1 [M+H]⁺

### Example M.49

### 7-(4-chloro-2-mesyl-phenyl)-2,7-diazaspiro[3.5]nonane 0.1:2 tosylic Acid

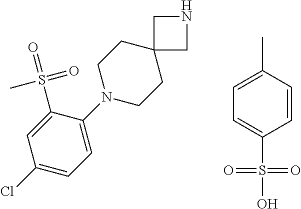 [SMILES: CS(=O)(=O)c1cc(Cl)ccc1N1CCC2(CC1)CNC2 ; Cc1ccc(S(=O)(=O)O)cc1]

A solution of 7-(4-chloro-2-mesyl-phenyl)-2,7-diazaspiro[3.5]nonane-2-carboxylic acid tert-butyl ester (37 mg, 0.089 mmol) in EtOAc (0.517 mL) was treated with PTSA (34.77 mg, 0.183 mmol), at 23° C. The mixture was stirred for 2 h at 80° C., before being cooled down and evaporated, affording the title compound (0.057 g; 92.7%) as an orange foam. MS (ESI): m/z=315.2 [M+H]⁺

### Step a): 1-bromo-4-chloro-2-mesyl-benzene

A suspension of 1-bromo-4-chloro-2-fluoro-benzene (CAS RN: 1996-29-8; 1.19 mL, 9.55 mmol) and NaSMe (702.77 mg, 10.03 mmol) in DMSO (21.33 mL) was stirred under argon at 100° C. for 3 h, before being cooled down. The mixture was poured on water and EtOAc and the layers were separated. The aqueous layer was extracted twice with EtOAc. The combined organic layers were washed twice with water, dried over Na₂SO₄, filtered and evaporated. The obtained intermediate (3.18 g, light yellow gum) was dissolved in dichloromethane (33.33 mL). The solution was cooled in an ice-bath and 3-chloroperoxybenzoic acid (3.06 g, 12.41 mmol) was added in portions. The reaction mixture was stirred at 23° C. for 18 h, and evaporated. Purification by FC (SiO₂; heptane/EtOAc) gave the title compound (1.26 g; 48.9%) as a colorless solid. MS (ESI): m/z=271.0 [M+H]⁺

### Step b): 7-(4-chloro-2-mesyl phenyl)-2,7-diazaspiro[3.5]nonane-2-carboxylic Acid Tert-Butyl Ester

A MW vial was charged with 1-bromo-4-chloro-2-mesyl-benzene (200 mg, 0.742 mmol), 2,7-diazaspiro[3.5]nonane-2-carboxylic acid tert-butyl ester (335.86 mg, 1.48 mmol), palladium (II) acetate (16.66 mg, 0.074 mmol), 2-(di-t-butylphosphino)biphenyl (22.14 mg, 0.074 mmol), sodium tert-butoxide (106.96 mg, 1.11 mmol) and toluene (1.56 mL), at 23° C. under Ar. The mixture was stirred in a MW at 130° C. for 30 min, diluted with EtOAc, and filtered. The filtrate was washed with water and brine. The combined organic layers were dried over Na₂SO₄, filtered and evaporated. Purification by FC (SiO₂; heptane/EtOAc) gave the title compound (0.018 g, 5.59%) as a colorless gum. MS (ESI): m/z=415.3 [M+H]⁺

### Example M30

### [3-(2-azaspiro[3.3]heptan-6-yloxy)phenyl]-imino-oxo-(trifluoromethyl)-λ⁶-sulfane; 4-methylbenzenesulfonic Acid

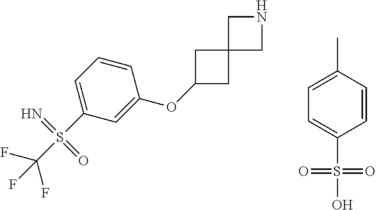 [SMILES: N=S(=O)(c1cccc(OC2CC3(CNC3)C2)c1)C(F)(F)F ; Cc1ccc(S(=O)(=O)O)cc1]

A solution of tert-butyl 6-[3-(trifluoromethylsulfonimidoyl)phenoxy]-2-azaspiro[3.3]heptane-2-carboxylate (840.0 mg, 2.0 mmol) in EtOAc (50 mL) was treated with PTSA (456.04 mg, 2.4 mmol), at 23° C. The mixture was stirred for another 16 h at this temperature, before being evaporated. Purification by RP-HPLC gave the title compound (253.6 mg, 24.48% yield) as a white solid. MS (ESI): m/z=321.2 [M+H]⁺

### Step a): 3-((trifluoromethylsulfonimidoyl)phenol

A solution of imino-oxo-[3-(4,4,5,5-tetramethyl-1,3,2-dioxaborolan-2-yl)phenyl]-(trifluoromethyl)λ6-sulfane (1.8 g, 5.37 mmol) in THE (90 mL) was treated with a solution of sodium hydrogen carbonate (0.45 g, 5.37 mmol) in water (9 mL), at 0° C. under air, followed by slow addition of hydrogen peroxide (6.09 g, 53.71 mmol). The mixture was stirred at room temperature for 1 h, before being washed with water and aqueous sodium hydrogensulfite. The organic layer was dried and evaporated, to give the title compound (1.0 g, 78.55% yield) as a light brown oil. MS (ESI): m/z=226.0 [M+H]⁺

### Step b): Tert-Butyl 6-[3-(trifluoromethylsulfonimidoyl)phenoxy]-2-azaspiro[3.3]heptane-2-carboxylate

A solution of tert-butyl 6-hydroxy-2-azaspiro[3.3]heptane-2-carboxylate (CAS RN: 1147557-97-8; 947.07 mg, 4.44 mmol) and 3-(trifluoromethylsulfonimidoyl)phenol (1.0 g, 4.44 mmol) in toluene (50 mL) was treated with cyanomethylenetributylphosphorane (2143.52 mg, 8.88 mmol), at 23° C. under Ar. The mixture was stirred for 16 h at 120° C., before being cooled down and evaporated. Purification by RP-HPLC gave the title compound (540.0 mg, 27.48% yield) as a light yellow solid. MS (ESI): m/z=321.0 [M−boc+H]⁺

### Example M.52

### N-(azetidin-3-ylmethyl)-1-(4-chloro-2-methoxy-phenyl)-N-methyl-methanamine; 4-methylbenzenesulfonic Acid

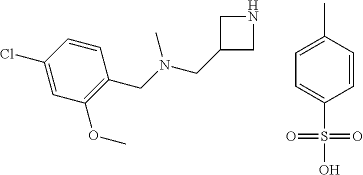

A solution of 3-[[(4-chloro-2-methoxy-benzyl)-methyl-amino]methyl]azetidine-1-carboxylic acid tert-butyl ester (246 mg, 0.693 mmol) in EtOAc (5.16 mL) was treated with PTSA (290 mg, 1.52 mmol), at 23° C. The mixture was stirred for 16 h at 80° C., before being cooled down and evaporated. Trituration with Et₂O gave the title compound (395.4 mg, 85.38%) as a colorless amorphous solid. MS (ESI): m/z=255.1 [M+H]⁺

### Step a): 4-chloro-2-methoxybenzaldehyde

A solution of (4-chloro-2-methoxyphenyl)methanol (1.00 g, 5.79 mmol) in CHCl₃ (12 mL) was added to a mixture of MnO₂ (5.04 g, 57.9 mmol) in CHCl₃ (12 mL), at 23° C. The mixture was stirred at reflux for 2 h, cooled down to 23° C., filtered over decalite, and evaporated, giving the title compound (877 mg, 88%) as a light yellow solid. MS (ESI): m/z=171.0 [M+H]⁺

### Step b): 3-[[(4-chloro-2-methoxy-benzyl)-methyl-amino]methyl]azetidine-1-carboxylic Acid Tert-Butyl Ester

A solution of 4-chloro-2-methoxy-benzaldehyde (200 mg, 1.17 mmol) and 3-(methylaminomethyl)azetidine-1-carboxylic acid tert-butyl ester (246.55 mg, 1.23 mmol) in dichloromethane (10 mL) and acetic acid (147.65 μL, 2.58 mmol) was stirred for 30 min at 23° C., before being treated with sodium triacetoxy borohydride (1.21 g, 5.73 mmol). The mixture was stirred for another 16 h at this temperature, before being poured into saturated aqueous NaHCO₃ (5 mL) and stirred for 1.5 h. The mixture was extracted with dichloromethane (2*10 mL). The organic layer was dried over Na₂SO₄, filtered and evaporated. Purification by FC (SiO₂; PE/MTBE) gave the title compound (246 mg, 58.54%) as a colorless viscous oil. MS (ESI): m/z=355.1 [M+H]⁺

### Synthesis of Non-Commercial Building Blocks

### Example C.1

### 4-bromo-2-methylsulfonyl-1-(trifluoromethyl)benzene

To a solution of 4-chloro-2-methylsulfanyl-1-(trifluoromethyl)benzene (1950 mg, 8.6 mmol) in 1,2-dichloroethane (20 mL), CH₃CN (20 mL) and water (40 mL) cooled with a water bath was added sodium periodate (3680 mg, 17.2 mmol) and ruthenium(III) chloride hydrate (19.4 mg, 0.090 mmol) at 0° C., then the mixture was stirred at 25° C. for 12 h. The reaction mixture was diluted with 40 mL water, and extracted with 50 mL DCM and 2×50 mL EtOAc. The combined organic layers were washed with 75 mL brine, dried over Na₂SO₄, filtered, and evaporated. Purification by FC (SiO₂; PE/EtOAc) gave the title compound (1800 mg, 73% yield) as a white solid. ¹H NMR (400 MHz, CHLOROFORM-d) δ ppm 8.32 (d, J=1.96 Hz, 1H) 7.87 (d, J=8.44 Hz, 1H) 7.76 (dd, J=8.44, 1.22 Hz, 1H) 3.22 ppm (s, 3H).

### Step a) 4-chloro-2-methylsulfanyl-1-(trifluoromethyl)benzene

A mixture of 4-chloro-2-fluoro-1-(trifluoromethyl)benzene (CAS: 94444-59-4) (5.0 g, 25.2 mmol) in DMF (50 mL) was added sodium methanethiolate (2.12 g, 30.2 mmol). The mixture was stirred at 50° C. for 2 h. The reaction mixture was diluted with 150 mL water, extracted with 2×75 mL EtOAc. The combined organic layers were washed with 100 mL brine, dried over Na₂SO₄, filtered, and evaporated. Purification by FC (SiO₂; PE/) gave the title compound (2 g, 31.5% yield) as a yellow solid. ¹H NMR (400 MHz, CHLOROFORM-d) δ ppm 7.55 (d, J=8.44 Hz, 1H) 7.30 (s, 1H) 7.20 (dd, J=8.38, 0.92 Hz, 1H) 2.52-2.57 ppm (m, 3H).

### Example C.2

### 5-bromo-2-[1-(trifluoromethyl)cyclopropyl]pyridine

To a solution of 5-bromo-2-[1-(trifluoromethyl)vinyl]pyridine (1100 mg, 4.36 mmol) in THF (40 mL) was added diphenyl(methyl)sulfonium tetrafluoroborate (1635 mg, 5.67 mmol). The suspension was stirred at room temperature for 0.5 h and finally a solution of NaHMDS (6.98 mL, 6.98 mmol, 1 M in THF) was added dropwise. The reaction mixture was heated at 25° C. for 1 h. The mixture was quenched by 10 mL MeOH and evaporated Purification by FC (SiO₂; PE/EtOAc) gave the title compound (900 mg, 78% yield) as a white solid. MS (ESI): m/z=266.3 [M+H]^{+ 1}H NMR (400 MHz, CHLOROFORM-d) δ=8.58 (d, J=2.3 Hz, 1H), 7.79 (dd, J=2.4, 8.4 Hz, 1H), 7.46 (d, J=8.4 Hz, 1H), 1.46-1.39 ppm (m, 4H).

### Step a) 5-bromo-2-[1-(trifluoromethyl)vinyl]pyridine

To a solution of 1-(trifluoromethyl)vinylboronic acid hexylene glycol ester (3374 mg, 15.2 mmol) and 2,5-dibromopyridine (3000 mg, 12.7 mmol), potassium carbonate (3500 mg, 25.3 mmol) in 1,4-Dioxane (60 mL) and water (12 mL) was added TETRAKIS[TRIPHENYLPHOSPHINE]PALLADIUM(0) (1463 mg, 1.27 mmol), the mixture was stirred at 80° C. under N₂ atmosphere for 12 h. The mixture was diluted with water (100 mL) and extracted with EtOAc (200 mL 3×), the combined organic phase was then washed with brine, dried over Na₂SO₄ and concentrated. Purification by FC (SiO₂; PE) gave the title compound as a light yellow oil. ¹H NMR (400 MHz, CHLOROFORM-d) δ=8.70 (d, J=2.2 Hz, 1H), 7.86 (dd, J=2.3, 8.6 Hz, 1H), 7.39 (d, J=8.6 Hz, 1H), 6.60 (d, J=1.7 Hz, 1H), 6.16 ppm (s, 1H).

### Example C.3

### 2-chloro-5-[1-(trifluoromethyl)cyclopropyl]pyrazine

To a solution of 2-chloro-5-[1-(trifluoromethyl)vinyl]pyrazine (1600 mg, 7.67 mmol) in THF (60 mL) was added diphenyl(methyl)sulfonium tetrafluoroborate (2873 mg, 9.97 mmol). The suspension was stirred at 0° C. for 0.5 h and finally a solution of NaHMDS (12.3 mL, 12.3 mmol, 1 M in THF) was added dropwise. The reaction mixture was heated at 25° C. for 1 h. Water (50 mL) was added in the reaction mixture and the aqueous phase was extracted with EtOAc (40 mL) twice. The organic layers were combined and washed with brine and dried over Na₂SO₄, and evaporated. Purification by FC (SiO₂; PE/EtOAc) gave the title compound (1500 mg, 63.89% yield) as a yellow oil. ¹H NMR (400 MHz, CHLOROFORM-d) δ=8.53 (s, 1H), 8.43 (d, J=1.2 Hz, 1H), 1.48-1.42 (m, 2H), 1.36 ppm (br s, 2H).

### Step a) 2-chloro-5-[1-(trifluoromethyl)vinyl]pyrazine

To a solution of 1-(trifluoromethyl)vinylboronic acid hexylene glycol ester (3443 mg, 15.5 mmol) and 2-bromo-5-chloropyrazine (3000 mg, 15.5 mmol), potassium carbonate (4290 mg, 31.0 mmol) in 1,4-Dioxane (60 mL) and water (6 mL) was added 1,1′-bis(diphenylphosphino)ferrocene-palladium(II)dichloride dichloromethane complex (1266 mg, 1.55 mmol), the mixture was stirred at 80° C. under N₂ atmosphere for 12 h, before being filtered and evaporated. Purification by FC (SiO₂; PE) gave the title compound (1600 mg, 49% yield) as a yellow solid. MS (ESI): m/z=209.3 [M+H]⁺

### Example C.4

### 3-bromo-6-[1-(trifluoromethyl)cyclopropyl]pyridazine

To a solution of 3-bromo-6-[1-(trifluoromethyl)vinyl]pyridazine (340 mg, 1.34 mmol) in THF (10 mL) was added diphenyl(methyl)sulfonium tetrafluoroborate (503 mg, 1.75 mmol). The suspension was stirred at 0° C. for 0.5 h and finally a solution of NaHMDS (2.15 mL, 2.15 mmol, 1 M in THF) was added dropwise. The reaction mixture was heated at 25° C. for 1 h. The mixture was quenched by 6 mL MeOH, and the reaction mixture was partitioned between EtOAc (20 mL) and water (40 mL) three times, and evaporated. Purification by prep-HPLC gave the title compound (280 mg, 78% yield) as a white solid. MS (ESI): m/z=267.3 [M+H]⁺

### Step a) 3-bromo-6-[1-(trifluoromethyl)vinyl]pyridazine

To a solution of 1-(trifluoromethyl)vinylboronic acid hexylene glycol ester (560 mg, 2.52 mmol) and 3,6-dibromopyridazine (500 mg, 2.1 mmol), potassium carbonate (581 mg, 4.2 mmol) in 1,4-Dioxane (10 mL) and Water (2 mL) was added 1,1′-bis(diphenylphosphino)ferrocene-palladium(II)dichloride dichloromethane complex (172 mg, 0.210 mmol), the mixture was stirred at 80° C. under N₂ atmosphere for 3 h. The mixture (combined with another duplicate reaction) was diluted with water (100 mL) and extracted with EtOAc (200 mL three times), the combined organic phase was then washed with brine, dried over Na₂SO₄ and concentrated. Purification by FC (SiO₂; PE) gave the title compound (300 mg, 56% yield) as a white crystalline solid. MS (ESI): m/z=253.3 [M+H]⁺

### Example C.5

### 5-bromo-2-[1-(trifluoromethyl)cyclopropyl]pyrimidine

To a solution of 5-bromo-2-[1-(trifluoromethyl)vinyl]pyrimidine (1900 mg, 7.51 mmol) in THF (60 mL) was added diphenyl(methyl)sulfonium tetrafluoroborate (2813 mg, 9.76 mmol). The suspension was stirred at room temperature for 0.5 h and finally a solution of NaHMDS (12.0 mL, 12.0 mmol) was added dropwise. The reaction mixture was heated at 25° C. for 1 h. The mixture was quenched by 10 mL MeOH, then the mixture was concentrated under reduced pressure to give the residue. Purification by FC (SiO₂; PE/EtOAc) gave the title compound (1400 mg, 69.8% yield) as a white solid. MS (ESI): m/z=269.0 [M+H]⁺

### Step a) 5-bromo-2-[1-(trifluoromethyl)vinyl]pyrimidine

To a solution of 1-(trifluoromethyl)vinylboronic acid hexylene glycol ester (4704 mg, 21.2 mmol) and 2,5-dibromopyrimidine (4200 mg, 17.7 mmol), potassium carbonate (4880 mg, 35.3 mmol) in 1,4-Dioxane (50 mL) and water (10 mL) was added 1,1′-bis(diphenylphosphino)ferrocene-palladium(II)dichloride dichloromethane complex (1441 mg, 1.77 mmol), the mixture was stirred at 80° C. under N² atmosphere for 12 h. The mixture was diluted with water (100 mL) and extracted with EtOAc (100 mL×3). The combined organic layers were washed with brine, dried over Na₂SO₄ and concentrated. Purification by FC (SiO₂; PE/EtOAc) gave the title compound (2.4 g, 53.7% yield) as a white solid. ¹H NMR (400 MHz, CHLOROFORM-d) δ=8.83 (s, 2H), 7.01 (d, J=1.8 Hz, 1H), 6.41 ppm (s, 1H).

### Example C.6

### [3-bromo-5-(trifluoromethyl)phenyl]-imino-methyl-oxo-λ⁶-sulfane

Under argon atmosphere, a 200 mL sealed tube was charged with 1-bromo-3-methylsulfinyl-5-(trifluoromethyl)benzene (10.0 g, 34.8 mmol), amino 4-nitrobenzoate; trifluoromethanesulfonic acid (28.9 g, 87.1 mmol), ferrous sulfate (1058 mg, 6.97 mmol), 1,10-phenanthroline (2511 mg, 13.9 mmol) and ACN (80 mL). Then, the reaction mixture was stirred at 40° C. for 72 h. After cooling to room temperature, the reaction mixture was quenched with a saturated NaHCO₃ solution (100 mL). The mixture was extracted with CH₂Cl₂ (150 mL×3). The combined organic layers were dried over anhydrous Na₂SO₄ and concentrated in vacuo. The product was purified by column chromatography to give the title compound (2.2 g, 7.28 mmol, 19.9% yield) as a light yellow solid. MS (ESI): m/z=302.0/304.0 [M+H]⁺

### Step a) 1-bromo-3-methylsulfinyl-5-(trifluoromethyl)benzene

1-bromo-3-methylsulfanyl-5-(trifluoromethyl)benzene (11.0 g, 40.6 mmol) was dissolved in trifluoroacetic acid (62.5 mL, 812 mmol), and the resulting mixture was cooled at 0° C. with an ice bath. Next, hydrogen peroxide (3.77 mL, 44.6 mmol) in water (4 mL) was added, and the mixture was stirred 12 h at room temperature. After completion, the mixture was concentrated and partitioned between DCM and NaHCO₃ sat. aq. solution. The organic layer was dried and concentrated to obtain the title compound (10.0 g, 34.83 mmol, 81.55% yield) as white solid. MS (ESI): m/z=287.0/289.0 [M+H]+

### Example C.7

### 1-chloro-4-dimethylphosphoryl-2-fluoro-benzene

To a solution of 4-chloro-3-fluoroiodobenzene (2000 mg, 7.8 mmol) in 1,4-dioxane (20 mL) was added TEA (2.17 mL, 15.6 mmol), methylphosphonoylmethane (CAS: 7211-39-4) (730 mg, 9.36 mmol), Xantphos (903 mg, 1.56 mmol) and Pd₂(dba)₃ (714 mg, 0.78 mmol) at 25° C. The reaction mixture was stirred at 70° C. for 12 h under N₂ atmosphere. The reaction mixture was filtered, the filtrate was concentrated under reduced pressure to give a residue. The crude product was purified by reverse flash (FA condition; MeCN:H₂O 0-30%) and lyophilized to give 1-chloro-4-dimethylphosphoryl-2-fluoro-benzene (1387 mg, 6.71 mmol, 86% yield) as light yellow powder. MS (ESI): m/z=207.0 [M+H]+

### Example C.8

### 1-bromo-3-dimethylphosphoryl-5-fluoro-benzene

To a solution of 1-bromo-3-fluoro-5-iodo-benzene (2.0 g, 6.65 mmol) in 1,4-dioxane (20 mL) was added TEA (2.32 mL, 16.6 mmol), methylphosphonoylmethane (CAS: 7211-39-4) (545 mg, 6.98 mmol), Xantphos (1923 mg, 3.32 mmol) and Pd₂(dba)₃ (609 mg, 0.66 mmol). The reaction mixture was stirred at 60° C. for 16 h under N₂ atmosphere. The reaction mixture was filtered and the filter was concentrated. The crude product was purified by reversed-phase HPLC (0.1% FA condition) and lyophilized to give 1-bromo-3-dimethylphosphoryl-5-fluoro-benzene (1.36 g, 5.42 mmol, 81.5% yield) as brown solid which confirmed by ¹H NMR. MS (ESI): m/z=251.1 [M+H]⁺; ¹H NMR (400 MHz, CHLOROFORM-d) δ=7.68-7.62 (m, 1H), 7.45-7.37 (m, 2H), 1.78 (s, 3H), 1.75 (s, 3H)

### Example C.9

### 1-(bromomethyl)-2-fluoro-4-(trifluoromethylsulfonyl)benzene

To a mixture of 2-fluoro-1-methyl-4-(trifluoromethylsulfonyl)benzene (4.2 g, 17.3 mmol) in trifluoromethylbenzene (80.0 mL, 646 mmol) was added N-BROMOSUCCINIMIDE (6.33 g, 35.6 mmol) and 2,2-AZOBIS(2-METHYLPROPIONITRILE) (5.84 g, 35.6 mmol). The reaction mixture was stirred at 90° C. for 12 h under N₂ atmosphere. The reaction mixture was concentrated under reduced pressure to give a residue. The residue was purified by flash chromatography on silica gel eluting with PE/EtOAc (9:1) to afford the title compound (2.5 g, 7.79 mmol, 44.9% yield) as a yellow oil. ¹H NMR (400 MHz, CHLOROFORM-d) δ=4.55 (s, 2H), 7.73-7.79 (m, 2H), 7.85 (dd, J=8.01, 1.28 Hz, 1H).

### Example C.10

### 3-bromo-5-[1-(trifluoromethyl)cyclopropyl]-1H-1,2,4-triazole

To a solution of 3-[1-(trifluoromethyl)cyclopropyl]-1H-1,2,4-triazole (5.07 g, 27.2 mmol) in N,N-dimethylformamide (25 mL) was added NBS (10.7 g, 59.8 mmol). The mixture was stirred at 60° C. for 15 h. The reaction mixture was poured into EtOAc and washed with water and brine. The organic layer was dried over Na₂SO₄ and concentrated in vacuo. The crude material was purified by flash chromatography (0% to 20% AcOEt in heptane) to afford the title compound (3.08 g, 42.0%) as a white solid. MS (ESI): m/z=255.9/257.9 [M+H]⁺

### Step a) (NE)-N-(dimethylaminomethylene)-1-(trifluoromethyl)cyclopropanecarboxamide

1-(trifluoromethyl)cyclopropanecarboxamide (5.0 g, 32.7 mmol) was dissolved in N,N-dimethylformamide dimethyl acetal (27.2 g, 30.4 mL, 229 mmol). The mixture was stirred at RT for 6 h. The reaction mixture was poured into EtOAc and washed with water (three times) and brine. The organic layer was dried over Na₂SO₄ and concentrated in vacuo to afford the title compound (6.5 g, 90.8%) as yellow amorphous solid. MS (ESI): m/z=209.1 [M+H]⁺

### Step b) 3-[1-(trifluoromethyl)cyclopropyl]-1H-1.2,4-triazole

To a ice cooled solution of (NE)-N-(dimethylaminomethylene)-1-(trifluoromethyl)cyclopropanecarboxamide (6.5 g, 28.1 mmol) in acetic acid (18 mL) was added hydrazine; hydrate (1.55 g, 1.5 mL, 30.9 mmol). The mixture was stirred at RT for 2 h, then at 50° C. for 2 h. The reaction mixture concentrated in vacuo and dissolved in EtOAc. NaOH 1 N aq. was added (pH 7-8). The mixture was washed with water and brine. The organic layer was dried over Na₂SO₄ and concentrated in vacuo to afford the title compound (5.07 g, 96.8%) as yellow solid. MS (ESI): m/z=178.1 [M+H]⁺

### Example C.11

### 4,5-dibromo-2-(2,2,2-trifluoroethyl)triazole

To a solution of 4,5-dibromo-2H-triazole (5.0 g, 22.0 mmol) in ACN (50 mL) was added cesium carbonate (7.18 g, 22.0 mmol). Then, 2,2,2-trifluoroethyl trifluoromethanesulfonate (5.12 g, 22.0 mmol) was added dropwise at 0° C. The reactional mixture was stirred for 16 h at RT. The reaction mixture was poured into water, EtOAc was added and the aqueous layer was extracted three times with EtOAc. Then, the combined organic layers were washed with water and brine, dried over Na₂SO₄, filtered off and evaporated. The crude product was purified by column chromatography (silica gel, PE/EtOAc 0-7%). The fractions containing the pure product were combined and evaporated to give 4,5-dibromo-2-(2,2,2-trifluoroethyl)triazole (5.3 g, 17.2 mmol, 77.9% yield) as yellow oil. ¹H NMR (400 MHz, CHLOROFORM-d) δ=4.95 (q, J=7.9 Hz, 2H)

### Example C.12

### 3-bromo-1-(2,2,2-trifluoroethyl)-1,2,4-triazole

To a solution of 3-bromo-1H-1,2,4-triazole (8000 mg, 54.1 mmol) in THE (80 mL) was added NaH (3.89 g, 97.3 mmol) at 0° C. After 0.5 h, was added 2,2,2-trifluoroethyl trifluoromethanesulfonate (25.1 g, 108 mmol) and the resulting mixture was stirred at 20° C. for 12 h. The reaction mixture was poured into water (200 mL) and extracted with EtOAc (200 mL×3). Combined extracts were washed with brine (150 mL), dried over Na₂SO₄, filtered, and concentrated under vacuum to give a residue. The residue was purified by prep-HPLC and was concentrated under reduced pressure to give the title compound (6000 mg, 26.1 mmol, 48.3% yield) as brown oil. MS (ESI): m/z=229.9 [M+H]⁺; ¹H NMR (400 MHz, CHLOROFORM-d) δ=12.49 (s, 1H), 9.14 (q, J=8.1 Hz, 2H)

### Example C.13

### 4-dimethylphosphoryl-2-methyl-pyrazole-3-carbaldehyde

To a solution of 4-iodo-2-methyl-pyrazole-3-carbaldehyde (CAS: 959986-66-4) (2000 mg, 8.47 mmol) in 1,4-dioxane (20 mL) was added TEA (2.36 mL, 17.0 mmol), dimethylphosphine oxide (794 mg, 10.2 mmol, 1.2 eq), Xantphos (981 mg, 1.69 mmol) and Pd₂(dba)₃ (776 mg, 0.85 mmol). The reaction mixture was stirred at 70° C. for 12 h under N₂ atmosphere. The reaction mixture was filtered, the filtrate was concentrated under reduced pressure to give a residue. The residue was purified by reverse flash chromatography (FA condition; MeCN:H₂O=0-30%) and lyophilized to give the title compound as light yellow solid. MS (ESI): m/z=187.0 [M+H]⁺; ¹H NMR (400 MHz, CHLOROFORM-d) δ ppm 1.81 (s, 3H) 1.84 (s, 3H) 4.25 (s, 3H) 7.60 (br d, J=1.83 Hz, 1H) 10.49 (s, 1H).

### Example C.14

### 1-bromo-4-dimethylphosphoryl-2-(trifluoromethyl)benzene

To methylphosphonoylmethane (1.85 g, 23.7 mmol), 1-bromo-4-iodo-2-(trifluoromethyl)benzene (CAS: 364-11-4) (8.3 g, 23.7 mmol), 9,9-dimethyl-4,5-bis(diphenylphosphino)xanthene (137 mg, 0.24 mmol) and triethylamine (3.86 mL, 27.7 mmol) was added 1,4-Dioxane (80 mL). Tris(dibenzylideneacetone)dipalladium (0) (108 mg, 0.12 mmol) was added, and the mixture was stirred at RT under Ar for 24 h. The reaction mixture was filtered through celite, and the filtrate was concentrated under reduced pressure. The residue was purified by FC (eluting with petroleum ether/MtBE with MtBE from 0-100%, further with MtBE/methanol with methanol from 0-25%) to afford 1-bromo-4-dimethylphosphoryl-2-(trifluoromethyl)benzene (50 mg, 0.170 mmol, 18.5% yield) as white semisolid. MS (ESI): m/z=301.0/303.0 [M+H]⁺

### Example C.15

### 1-bromo-3-dimethylphosphoryl-5-(trifluoromethyl)benzene

Methylphosphonoylmethane (2224 mg, 28.5 mmol), 1-bromo-3-iodo-5-(trifluoromethyl)benzene (CAS: 481075-59-6) (5.0 g, 14.3 mmol), 9,9-dimethyl-4,5-bis(diphenylphosphino)xanthene (206 mg, 0.36 mmol) and tris(dibenzylideneacetone)dipalladium (0) (130. mg, 0.14 mmol) were dissolved in 1,4-Dioxane (50 mL) and triethylamine (2.98 mL, 21.4 mmol). The reaction mixture was stirred at 25° C. under argon for 48 h. The reaction mixture was concentrated under reduced pressure. The residue was partitioned between ethyl acetate and water. The organic layer was washed with brine. The extract was dried over sodium sulfate and evaporated. The crude product was purified by flash chromatography to afford the title compound (1.9 g, 6.31 mmol, 42.5% yield) as an orange solid. MS (ESI): m/z=301.0 [M+H]⁺.

### Example C.16

### 2-benzyloxy-5-bromo-3-(difluoromethyl)pyridine

To the ice-water cooled stirred solution of 5-bromo-3-(difluoromethyl)-2-fluoro-pyridine (CAS: 1805222-04-1) (1.5 g, 6.64 mmol) and benzyl alcohol (754 mg, 6.97 mmol) in THE (45 mL), potassium tert-butoxide (819 mg, 7.3 mmol) was added in one portion. Then RM was stirred at 25° C. for 12 h and evaporated in vacuum. The obtained residue was partitioned between water (30 mL) and EtOAc (50 mL). The organic layer was dried over Na₂SO₄ and evaporated to give 2-benzyloxy-5-bromo-3-(difluoromethyl)pyridine (1.9 g, 6.05 mmol, 86.6% yield) as white solid (95% NMR). MS (ESI): m/z=314.0 [M+H]⁺.

### Example C.17

### 3-bromo-5-[1-(trifluoromethyl)cyclopropyl]-1H-pyrazole

To a solution of 5-[1-(trifluoromethyl)cyclopropyl]-1H-pyrazol-3-amine (2.3 g, 12.0 mmol) in ACN (40 mL) was added a solution of isopentyl nitrite (1.55 g, 13.2 mmol) in ACN (5 mL) dropwise at 0° C. The reaction was stirred at 0° C. for 1 h. Then CuBr₂ (1.61 g, 7.22 mmol) was added into the mixture at 0° C. and the resulting mixture was stirred at 20° C. for 12 h. The reaction mixture was concentrated under reduced pressure to give a residue. The residue was purified by reversed-phase HPLC (water(0.1% FA)-ACN]). The purified solution was lyophilized to afford the title compound (740 mg, 2.9 mmol, 24.1% yield) as a light green solid. MS (ESI): m/z=257.2 [M+H]⁺.

### Example C.18

### 4-[1-(trifluoromethyl)cyclopropyl]-1H-imidazole

2-bromo-1-[1-(trifluoromethyl)cyclopropyl]ethanone (5.0 g, 21.6 mmol) was added to methanamide (50.0 mL, 1259 mmol) and the mixture was stirred at 180° C. for 2 h. After the reaction was completed, 500 mL of water was added, and the mixture was washed three times with dichloromethane (100 mL×3), then the aqueous phase was adjusted to pH 8 with a 1 M aqueous NaOH solution, and the aqueous phase was extracted twice with dichloromethane (50 mL×2). The organic phase was dried and concentrated to give 4-[1-(trifluoromethyl)cyclopropyl]-1H-imidazole (800 mg, 4.54 mmol, 15.5% yield) as brown solid. MS (ESI): m/z=177.2 [M+H]⁺.

### Example C.19

### 2-benzyloxy-3-bromo-5-(difluoromethyl)pyridine

To the ice-water cooled stirred solution of 3-bromo-5-(difluoromethyl)-2-fluoro-pyridine (1.4 g, 6.19 mmol) and benzyl alcohol (703 mg, 6.5 mmol) in THE (40 mL), potassium tert-butoxide (765 mg, 6.81 mmol) was added in one portion. The reaction mixture was stirred at 25° C. for 18 h and evaporated in vacuum. The obtained residue was partitioned between water (40 mL) and EtOAc (200 mL). The organic layer was dried over Na₂SO₄ and evaporated in vacuum to give the title compound (1.7 g, 5.41 mmol, 83% yield) as light yellow microcrystals. MS (ESI): m/z=314.0/316.0 [M+H]⁺

### Example C.20

### [4-bromo-2-(trifluoromethyl)phenyl]-imino-methyl-oxo-λ⁶-sulfane

Iodobenzene diacetate (23.8 g, 73.8 mmol) and ammonium carbamate (3.6 g, 46.1 mmol) were added to a solution of 4-bromo-1-methylsulfanyl-2-(trifluoromethyl)benzene (CAS: 300356-31-4) (5.0 g, 18.4 mmol) in 2,2,2-trifluoroethan-1-ol (50.0 mL). The reaction mixture was stirred at RT for 24 h. After completion, the solvent was evaporated and the residue was diluted between water and EtOAc. The organic layer was dried over Na₂SO₄, filtered and concentrated to yield the title compound (7.8 g, 25.8 mmol, 4.2% yield) as dark brown oil. MS (ESI): m/z=302.0 [M+H]⁺.

### Example C.21

### [2-bromo-5-(trifluoromethyl)phenyl]-imino-methyl-oxo-λ⁶-sulfane

Under an argon atmosphere, a 30 mL sealed tube was charged with 1-bromo-2-methylsulfinyl-4-(trifluoromethyl)benzene (2.5 g, 8.71 mmol), amino 4-nitrobenzoate; trifluoromethanesulfonic acid (7232 mg, 21.8 mmol), ferrous sulfate (265 mg, 1.74 mmol), 1,10-phenanthroline (628 mg, 3.48 mmol) and ACN (20 mL). Then, the reaction mixture was stirred at 30° C. for 48 h. After cooling to room temperature, the reaction mixture was quenched with a saturated NaHCO₃ solution (5 mL). The organic layer was separated from the aqueous one, which was extracted with CH₂Cl₂ (5 mL×3). The combined organic layers were dried over anhydrous Na₂SO₄ and concentrated in vacuo. The product was purified by flash chromatography (0-50% acetonitrile/chloroform, then 10-40% acetonitrile/water) to give the title compound (900 mg, 2.98 mmol, 31% yield) as light yellow solid. LCMS 302.0/304.0 [M+H]⁺

### Step a) 1-bromo-2-methylsulfinyl-4-(trifluoromethyl)benzene

1-bromo-2-methylsulfanyl-4-(trifluoromethyl)benzene (CAS: 142994-07-8) (10.0 g, 28.4 mmol) was dissolved in trifluoroacetic acid (100 mL, 1298 mmol), and the resulting mixture was cooled at 0° C. with an ice bath. Next, hydrogen peroxide (2.4 mL, 28.4 mmol) in water (3.24 mL) was added, and the mixture was stirred 18 h at RT. After completion, the mixture was concentrated and diluted between DCM and NaHCO₃ aq. 1 M solution. The organic layer was dried and concentrated, then was purified by flash chromatography (hexane/MTBE 20-42%) to obtain the title compound (6.0 g, 20.9 mmol, 73.6% yield) as light yellow oil; MS (ESI): m/z=287.0/288.9 [M+H]⁺

### Example C.22

### 1-(3,3,3-trifluoropropyl)pyrazole-3-carbaldehyde

To a solution of 1H-pyrazole-3-carbaldehyde (5.0 g, 52.0 mmol) in N,N-dimethylformamide (100 mL) was added 1,1,1-trifluoro-3-iodopropane (17.5 g, 78.1 mmol), Cs₂CO₃ (33.9 g, 104 mmol, 2.0 eq) and 18-crown-6 (6.88 g, 26.02 mmol, 0.5 eq). The reaction mixture was stirred at 90° C. for 3 h. Then 1,1,1-trifluoro-3-iodopropane (17.5 g, 78.1 mmol) and 18-crown-6 (6.88 g, 26.0 mmol) was added into the mixture and the resulting mixture was stirred at 90° C. for another 60 h. The reaction mixture was poured into water (200 mL) and then extracted with ethyl acetate (100 mL×3). The combined organic layers were washed with brine (100 mL), dried over Na₂SO₄, filtered and concentrated under reduced pressure to give a residue. The residue was purified by flash chromatography on silica gel eluting with PE/EtOAc (3:1) to afford the product 1-(3,3,3-trifluoropropyl)pyrazole-3-carbaldehyde (1.15 g, 5.99 mmol, 12% yield) as a yellow oil. MS (ESI): m/z=193.1 [M+H]; ¹H NMR (400 MHz, CHLOROFORM-d) δ=9.96 (s, 1H), 7.48 (d, J=2.1 Hz, 1H), 6.81 (d, J=2.4 Hz, 1H), 4.46 (t, J=7.2 Hz, 2H), 2.82 (tq, J=7.2, 10.2 Hz, 2H).

### Example C.23

### 2-[[2-bromo-4-trifluoromethyl)imidazol-1-yl]methoxy]ethyl-trimethyl-silane

A mixture of trimethyl-[2-[[4-(trifluoromethyl)imidazol-1-yl]methoxy]ethyl]silane (30.0 g, 113 mmol), N-bromosuccinimide (26.1 g, 146 mmol) and AIBN (3.7 g, 22.5 mmol) in Carbon tetrachloride (300 mL) was stirred at 60° C. for 12 h. The reaction mixture was filtered, the filtrate was concentrated under reduced pressure to give a residue. The residue was purified by reverse flash (FA condition; MeCN:H₂O 0-70%) and lyophilized to afford the title compound (21.0 g, 60.8 mmol, 54% yield) as a yellow oil. MS (ESI): m/z=346.9 [M+H]⁺; ¹H NMR (400 MHz, CHLOROFORM-d) δ=7.45 (s, 1H), 5.30 (s, 2H), 3.66-3.53 (m, 2H), 1.02-0.89 (m, 2H), 0.01 (s, 9H)

### Example C.24

### 1-bromo-3-fluoro-5-(trifluoromethylsulfanyl)benzene

To a solution of 1-bromo-3-fluoro-5-iodo-benzene (2000 mg, 6.65 mmol) in ACN (10 mL) were added 2,2′-bipyridine (1038 mg, 6.65 mmol), CuI (1266 mg, 6.65 mmol) and trifluoromethylsulfanylsilver (1667 mg, 7.98 mmol). The mixture was stirred at 90° C. for 12 h under N₂ balloon. The reaction mixture was filtered to get 1-bromo-3-fluoro-5-(trifluoromethylsulfanyl)benzene (1.8 g, 6.54 mmol, 98.45% yield) as a green liquid solution. The filtrate was used in the next step of the reaction directly. (NMR of solution concentrated in vacuo to give crude product: ¹H NMR (400 MHz, CHLOROFORM-d) δ=7.69 (d, J=0.6 Hz, 1H), 7.48-7.37 (m, 1H), 7.32-7.21 (m, 1H))

### Example C.25

### 2-bromo-3-methoxy-5-(trifluoromethyl)pyrazine

To a mixture of 3-methoxy-5-(trifluoromethyl)pyrazin-2-amine (1.6 g, 8.28 mmol) and CuBr (1.42 g, 9.94 mmol) in MeCN (15 mL) was added a solution of tert-butyl nitrite (1.28 g, 12.4 mmol) in MeCN (5 mL) at 25° C., then the reaction was stirred at 70° C. under N₂ atmosphere for 12 h. The reaction mixture was filtered, the filtrate was concentrated under reduced pressure at room temperature to give a residue. The residue was purified by column chromatography (ethyl acetate:petroleum ether 0-10%) and concentrated under reduced pressure at room temperature to give the title compound (800 mg, 3.11 mmol, 38% yield) as a colorless oil. MS (ESI): m/z=256.9 [M+H]⁺. NOTE:The product was volatile.

### Example C.26

### 6-bromo-2-isopropoxy-3-(trifluoromethyl)pyridine

2-propanol (0.69 mL, 9.02 mmol) was dissolved in dry DMF (10 mL), obtained solution was cooled to 0° C. and sodium hydride, 60% in oil (0.49 g, 12.3 mmol) was added portionwise. After 30 minutes stirring at 0-10° C., 6-bromo-2-fluoro-3-(trifluoromethyl)pyridine (2.0 g, 8.2 mmol) was dissolved in dry DMF (10 mL) and cooled to 0° C. Previously obtained sodium isopropylate solution in DMF was added dropwise at 0-10° C. and reaction mixture was stirred at 20° C. for 16 h. The reaction mixture was quenched with saturated NH₄Cl solution (150 mL) and extracted with MTBE (150 mL). Organic extract was washed with brine (2×150 mL), dried over anhydrous sodium sulfate and evaporated in vacuum to obtain the title compound (2.2 g, 7.74 mmol, 80% yield), which was used in the next step without additional purification.

### Example 997

A compound of formula (I) can be used in a manner known per se as the active ingredient for the production of tablets of the following composition:

| | Per tablet |
|---|---|
| Active ingredient | 200 mg |
| Microcrystalline cellulose | 155 mg |
| Corn starch | 25 mg |
| Talc | 25 mg |
| Hydroxypropylmethylcellulose | 20 mg |
| | 425 mg |

### Example 998

A compound of formula (I) can be used in a manner known per se as the active ingredient for the production of capsules of the following composition:

| | Per capsule |
|---|---|
| Active ingredient | 100.0 mg |
| Corn starch | 20.0 mg |
| Lactose | 95.0 mg |
| Talc | 4.5 mg |
| Magnesium stearate | 0.5 mg |
| | 220.0 mg |

LENGTHY TABLES
The patent application contains a lengthy table section. A copy of the table is available in electronic form from the USPTO web site (https://seqdata.uspto.gov/?pageRequest=docDetail&DocID=US20250099448A1). An electronic copy of the table will also be available from the USPTO upon request and payment of the fee set forth in 37 CFR 1.19(b)(3).

1. A compound of formula (I) 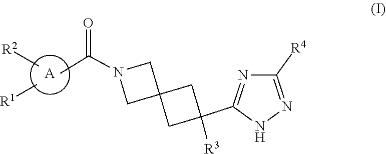
or a pharmaceutically acceptable salt thereof, wherein:
A is selected from: 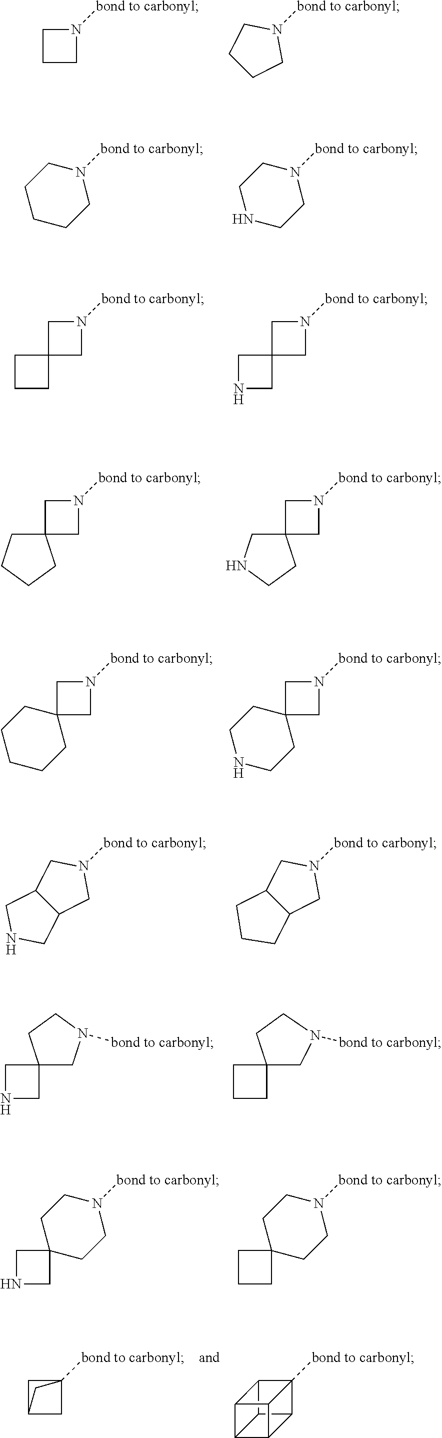
B is selected from C₆-C₁₄-aryl, C₃-C₁₀-cycloalkyl, 5- to 14-membered heteroaryl, and 3- to 14-membered heterocyclyl;
C is selected from C₆-C₁₄-aryl, C₃-C₁₀-cycloalkyl, 3- to 14-membered heterocyclyl, and 5- to 14-membered heteroaryl;
L¹ is selected from a covalent bond, carbonyl, —CR^{8a}R^{8b}—, —CH₂O—, —OCH₂—, —CH₂NR^{13a}—, —NR^{13b}CH₂—, —CH₂CH₂—, —CH₂NR^{13c}CH₂—, —CH₂NHCO—, —O—, —NH—, —SO₂NH—, —NHSO₂—, —SO₂NHCH₂—, —CH₂NHSO₂—, —SO₂—, —CH₂SO₂—, —SO₂CH₂—, —NHC(O)—, —C(O)NH—, and 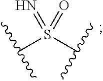 [SMILES: *S(*)(=N)=O]
L² is selected from a covalent bond, —CR^{8a}R^{8b}—, —NH—, —N(C₁-C₆-alkyl), —O—, —CH₂NH—, —NHCH₂—, —CH₂O—, —OCH₂—, —SO₂—, —CH₂SO₂—, and —SO₂CH₂—;
X is NH or O;
R¹ is selected from a group 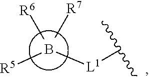
C₁-C₆-alkyl-SO₂—NH—, C₁-C₆-alkyl-SO₂—, and halo-C₁-C₆-alkyl-C(O)—;
R² is selected from hydrogen and hydroxy;
R³ is selected from hydrogen, halogen, cyano, C₁-C₆-alkyl, C₁-C₆-alkoxy, and hydroxy;
R⁴ is selected from hydrogen and a group —C(R^{4a}R^{4b}R^{4c});
R^{4a} is selected from C₁-C₆-alkyl and halo-C₁-C₆-alkyl; R^{4b} is selected from hydrogen and hydroxy; and R^{4c} is hydrogen; or
R^{4a}, R^{4b}, and R^{4c} are each independently selected from hydrogen and halogen; or
R^{4a} and R^{4b}, taken together with the carbon atom to which they are attached, form a C₃-C₁₀-cycloalkyl or a 3- to 14-membered heterocyclyl; and R^{4c} is selected from hydrogen, hydroxy, amino, and halogen; wherein said C₃-C₁₀-cycloalkyl and 3- to 14-membered heterocyclyl are optionally substituted with 1-2 halogen substituents;
R⁵ is selected from hydrogen, hydroxy, halogen, cyano, C₁-C₆-alkyl, C₁-C₆-alkoxy, halo-C₁-C₆-alkyl, hydroxy-C₁-C₆-alkyl, hydroxy-halo-C₁-C₆-alkyl, carboxy-C₁-C₆-alkyl, halo-C₁-C₆-alkoxy, oxo, NH₂—SO₂—, amino, C₁-C₆-alkyl-NH—, (C₁-C₆-alkyl)₂N—, carbamoyl, C₁-C₆-alkyl-NH—C(O)—, (C₁-C₆-alkyl)₂N—C(O)—, carboxy, C₁-C₆-alkoxycarbonyl, C₁-C₆-alkoxycarbonyl-C ₁—C₆-alkyl-, (C₁-C₆-alkyl)₂P(O)—, (C₁-C₆-alkyl)₂P(O)—C₁-C₆-alkyl-, a group 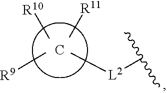
and a group 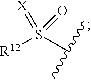
R⁶ is selected from hydrogen, halogen, cyano, oxo, C₁-C₆-alkoxy, halo-C₁-C₆-alkoxy, halo-C₁-C₆-alkyl, and C₁-C₆-alkyl-SO₂—;
R⁷ is selected from hydrogen and C₁-C₆-alkyl;
R^{8a} is selected from hydrogen, halogen, C₁-C₆-alkyl, and carbamoyl;
R^{8b} is selected from hydrogen and halogen;
R⁹ is selected from hydrogen, halogen, cyano, hydroxy, oxo, carboxy, C₁-C₆-alkoxycarbonyl, amino, C₁-C₆-alkyl-NH—, (C₁-C₆-alkyl)₂N—, carbamoyl, C₁-C₆-alkyl-NH—C(O)—, C₁-C₆-alkyl, hydroxy-C₁-C₆-alkyl, halo-C₁-C₆-alkyl, C₁-C₆-alkoxy-C₁-C₆-alkyl, C₁-C₆-alkoxy, halo-C₁-C₆-alkoxy, C₃-C₁₀-cycloalkyl, cyano, C₁-C₆-alkyl-SO₂—, halo-C₁-C₆-alkyl-SO₂—, and 5- to 14-membered heteroaryl; wherein said C₃-C₁₀-cycloalkyl and 5- to 14-membered heteroaryl is optionally substituted with one substituent selected from C₁-C₆-alkyl and halo-C₁-C₆-alkyl; or
R⁹ and R⁵ together form a group 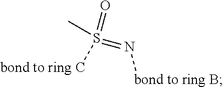
R¹⁰ is selected from hydrogen, halogen, C₁-C₆-alkyl, halo-C₁-C₆-alkyl, and oxo;
R¹¹ is selected from hydrogen and halogen;
R¹² is selected from amino, C₁-C₆-alkyl and halo-C₁-C₆-alkyl; and
R^{13a}, R^{13b}, and R^{13c} are each independently selected from hydrogen, C₁-C₆-alkyl, C₃-C₁₀-cycloalkyl, C₃-C₁₀-cycloalkyl-C₁-C₆-alkyl-.

2. The compound of formula (I) according to claim 1, or a pharmaceutically acceptable salt thereof, wherein A is selected from:

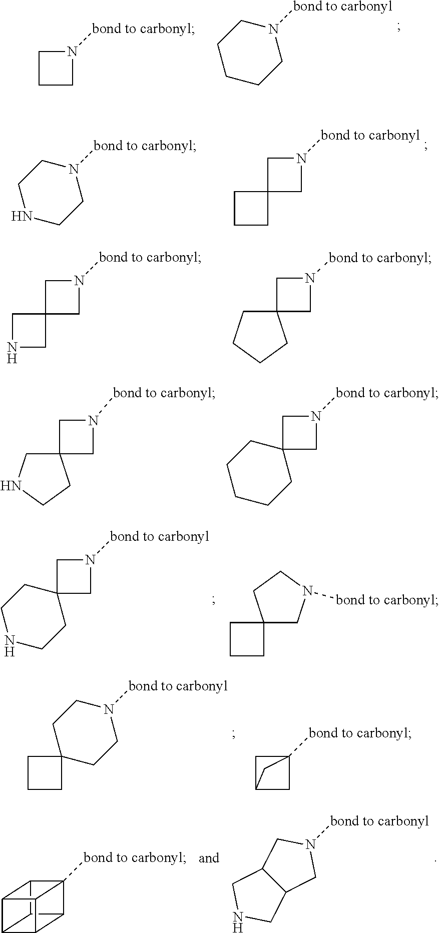

3. The compound of formula (I) according to claim 2, or a pharmaceutically acceptable salt thereof, wherein A is selected from:

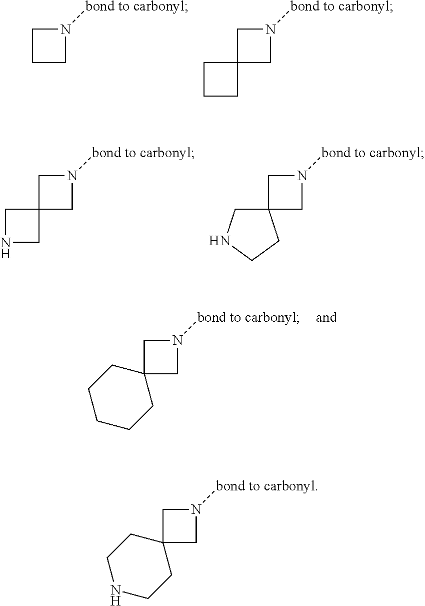

4. The compound of formula (I) according to any one of claims 1 to 3, or a pharmaceutically acceptable salt thereof, wherein:
B is selected from C₆-C₁₄-aryl, C₃-C₁₀-cycloalkyl, 5- to 14-membered heteroaryl, and 3- to 14-membered heterocyclyl;
C is selected from C₆-C₁₄-aryl, C₃-C₁₀-cycloalkyl, 3- to 14-membered heterocyclyl, and 5- to 14-membered heteroaryl;
L¹ is selected from a covalent bond, carbonyl, —CR^{8a}R^{8b}—, —CH₂O—, —OCH₂—, —CH₂NR^{13a}—, —NR^{13b}CH₂—, —CH₂CH₂—, —CH₂NR^{13c}CH₂—, —CH₂NHCO—, —O—, —NH—, —SO₂NH—, —NHSO₂—, —SO₂NHCH₂—, —CH₂NHSO₂—, —SO₂—, —NHC(O)—H, —C(O)NH—, and 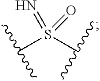 [SMILES: *S(*)(=N)=O]
L² is selected from a covalent bond, —CR^{8a}R^{8b}—, —NH—, —N(C₁-C₆-alkyl)-, —O—, —CH₂NH—, —NHCH₂—, —CH₂O—, —OCH₂—, —SO₂—, and —CH₂SO₂—;
X is NH or O;
R¹ is selected from a group 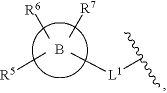
C₁-C₆-alkyl-SO₂—NH—, C₁-C₆-alkyl-SO₂—, and halo-C₁-C₆-alkyl-C(O)—;
R² is selected from hydrogen and hydroxy;
R⁵ is selected from hydrogen, hydroxy, halogen, cyano, C₁-C₆-alkyl, C₁-C₆-alkoxy, halo-C₁-C₆-alkyl, hydroxy-C₁-C₆-alkyl, hydroxy-halo-C₁-C₆-alkyl, carboxy-C₁-C₆-alkyl, halo-C₁-C₆-alkoxy, oxo, NH₂—SO₂—, C₁-C₆-alkyl-NH—, carbamoyl, C₁-C₆-alkyl-NH—C(O)—, carboxy, C₁-C₆-alkoxycarbonyl, C₁-C₆-alkoxycarbonyl-C₁-C₆-alkyl-, (C₁-C₆-alkyl)₂P(O)—, (C₁-C₆-alkyl)₂P(O)—C₁-C₆-alkyl-, a group 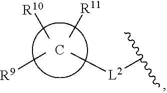
and a group 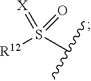
R⁶ is selected from hydrogen, halogen, cyano, oxo, C₁-C₆-alkoxy, halo-C₁-C₆-alkoxy, halo-C₁-C₆-alkyl, and C₁-C₆-alkyl-SO₂—;
R⁷ is selected from hydrogen and C₁-C₆-alkyl;
R^{8a} is selected from hydrogen, halogen, C₁-C₆-alkyl, and carbamoyl;
R^{8b} is selected from hydrogen and halogen;
R⁹ is selected from hydrogen, halogen, cyano, hydroxy, oxo, carboxy, C₁-C₆-alkoxycarbonyl, C₁-C₆-alkyl-NH—, carbamoyl, C₁-C₆-alkyl-NH—C(O)—, C₁-C₆-alkyl, hydroxy-C₁-C₆-alkyl, halo-C₁-C₆-alkyl, C₁-C₆-alkoxy-C₁-C₆-alkyl, C₁-C₆-alkoxy, halo-C₁-C₆-alkoxy, C₃-C₁₀-cycloalkyl, cyano, C₁-C₆-alkyl-SO₂—, halo-C₁-C₆-alkyl-SO₂—, and 5- to 14-membered heteroaryl; wherein said C₃-C₁₀-cycloalkyl and 5- to 14-membered heteroaryl is optionally substituted with one substituent selected from C₁-C₆-alkyl and halo-C₁-C₆-alkyl; or
R⁹ and R⁵ together form a group 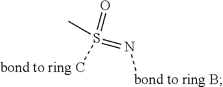
R¹⁰ is selected from hydrogen, halogen, C₁-C₆-alkyl, halo-C₁-C₆-alkyl, and oxo;
R¹¹ is selected from hydrogen and halogen;
R¹² is selected from amino, C₁-C₆-alkyl and halo-C₁-C₆-alkyl;
R^{13a} is selected from hydrogen, C₁-C₆-alkyl, C₃-C₁₀-cycloalkyl, C₃-C₁₀-cycloalkyl-C₁-C₆-alkyl-;
R^{13b} is hydrogen; and
R^{13c} is selected from hydrogen and C₁-C₆-alkyl.

5. The compound of formula (I) according to claim 4, or a pharmaceutically acceptable salt thereof, wherein:
B is selected from C₆-C₁₄-aryl, C₃-C₁₀-cycloalkyl, 5- to 14-membered heteroaryl, and 3- to 14-membered heterocyclyl;
C is selected from C₃-C₁₀-cycloalkyl and 5- to 14-membered heteroaryl;
L¹ is selected from a covalent bond, —CR^{8a}R^{8b}—, and —CH₂O—;
L² is selected from a covalent bond, —NH—, and —CH₂NH—;
X is NH or O;
R¹ is a group 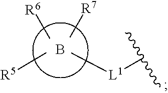
R² is hydrogen;
R⁵ is selected from halogen, cyano, halo-C₁-C₆-alkyl, oxo, halo-C₁-C₆-alkoxy, C₁-C₆-alkyl-SO₂—, halo-C₁-C₆-alkyl-SO₂—, a group 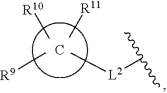
and a group 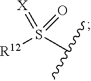
R⁶ is selected from hydrogen, halogen, cyano, and halo-C₁-C₆-alkyl;
R⁷ is selected from hydrogen and C₁-C₆-alkyl;
R^{8a} is selected from hydrogen and halogen;
R^{8b} is selected from hydrogen and halogen;
R⁹ is selected from hydrogen, C₁-C₆-alkyl and halo-C₁-C₆-alkyl;
R¹⁰ is selected from hydrogen, C₁-C₆-alkyl, and halo-C₁-C₆-alkyl;
R¹¹ is hydrogen; and
R¹² is halo-C₁-C₆-alkyl.

6. The compound of formula (I) according to claim 5, or a pharmaceutically acceptable salt thereof, wherein:
B is selected from phenyl, bicyclo[1.1.1]pentyl, pyridyl, oxazolyl, pyrimidinyl, pyrazinyl, pyridazinyl, 1,2,4-thiadiazolyl, pyrazolyl, imidazolyl, 1H-1,2,4-triazolyl, triazol-2-yl, 2H-triazolyl, isoxazolyl, 1,2,4-oxadiazolyl, 1,3,4-oxadiazolyl, 1,2-dihydropyridyl, and 1,2-dihydropyrazinyl;
C is selected from cyclopropyl, pyrazolyl, 1,3,4-oxadiazolyl, 1,3,4-thiadiazolyl, and pyrazinyl;
L¹ is selected from a covalent bond, —CR^{8a}R^{8b}—, and —CH₂O—;
L² is selected from a covalent bond, —NH—, and —CH₂NH—;
X is NH or O;
R¹ is a group 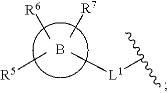
R² is hydrogen;
R⁵ is selected from chloro, fluoro, cyano, CF₃, 2,2,2-trifluoroethyl, CF₃O, oxo, methylsulfonyl, CF₃—SO₂—, a group 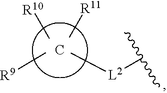
and a group 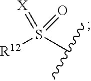
R⁶ is selected from hydrogen, fluoro, cyano, and CF₃;
R⁷ is selected from hydrogen and methyl;
R^{8a} is selected from hydrogen and fluoro;
R^{8b} is selected from hydrogen and fluoro;
R⁹ is selected from hydrogen, methyl, 2,2-dimethylpropyl, CHF₂, and CF₃;
R¹⁰ is selected from hydrogen, methyl, and CF₃;
R¹¹ is hydrogen; and
R¹² is CF₃.

7. The compound of formula (I) according to any one of claims 1 to 6, or a pharmaceutically acceptable salt thereof, wherein R³ is hydrogen.

8. The compound of formula (I) according to any one of claims 1 to 7, or a pharmaceutically acceptable salt thereof, wherein:
R⁴ is a group —C(R^{4a}R^{4b}R^{4c}); wherein
R^{4a}, R^{4b}, and R^{4c} are each independently halogen; or
R^{4a} and R^{4b}, taken together with the carbon atom to which they are attached, form a C₃-C₁₀-cycloalkyl; and R^{4c} is selected from hydrogen, hydroxy, and halogen; wherein said C₃-C₁₀-cycloalkyl is optionally substituted with 1-2 halogen substituents.

9. The compound of formula (I) according to claim 8, or a pharmaceutically acceptable salt thereof, wherein:
R⁴ is a group —C(R^{4a}R^{4b}R^{4c}); wherein
R^{4a}, R^{4b}, and R^{4c} are each independently fluoro; or
R^{4a} and R^{4b}, taken together with the carbon atom to which they are attached, form a cyclopropyl or a cyclobutyl; and R^{4c} is selected from hydrogen, hydroxy, and fluoro; wherein said cyclopropyl is optionally substituted with 1-2 fluoro substituents.

10. The compound of formula (I) according to claim 1, or a pharmaceutically acceptable salt thereof, wherein:
A is selected from: 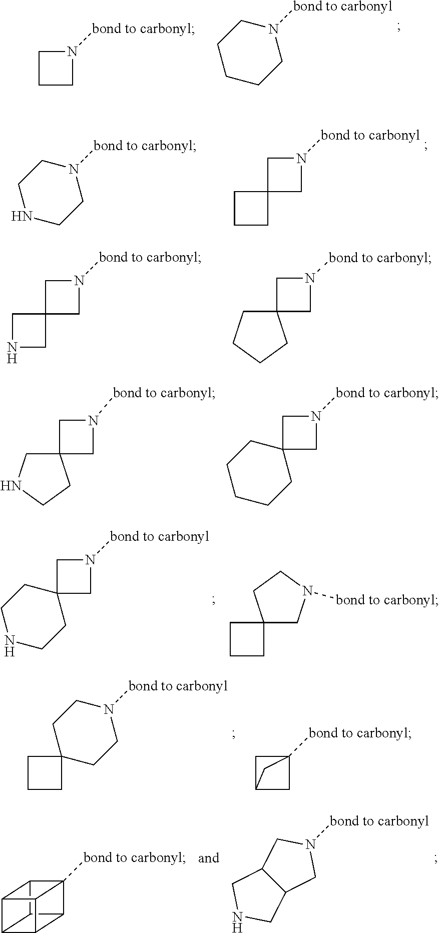
B is selected from C₆-C₁₄-aryl, C₃-C₁₀-cycloalkyl, 5- to 14-membered heteroaryl, and 3- to 14-membered heterocyclyl;
C is selected from C₆-C₁₄-aryl, C₃-C₁₀-cycloalkyl, 3- to 14-membered heterocyclyl, and 5- to 14-membered heteroaryl;
L¹ is selected from a covalent bond, carbonyl, —CR^{8a}R^{8b}—, —CH₂O—, —OCH₂—, —CH₂NR^{13a}—, —NR^{13b}CH₂—, —CH₂CH₂—, —CH₂NR^{13c}CH₂—, —CH₂NHCO—, —O—, —NH—, —SO₂NH—, —NHSO₂—, —SO₂NHCH₂—, —CH₂NHSO₂—, —SO₂—, —NHC(O)—, —C(O)NH—, and 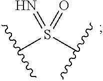 [SMILES: *S(*)(=N)=O]
L² is selected from a covalent bond, —CR^{8a}R^{8b}—, —NH—, —N(C₁-C₆-alkyl)-, —O—, —CH₂NH—, —NHCH₂—, —CH₂O—, —OCH₂—, —SO₂—, and —CH₂SO₂—;
X is NH or O;
R¹ is selected from a group 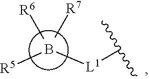
C₁-C₆-alkyl-SO₂—NH—, C₁-C₆-alkyl-SO₂—, and halo-C₁-C₆-alkyl-C(O)—;
R² is selected from hydrogen and hydroxy;
R³ is hydrogen;
R⁴ is selected from hydrogen and a group —C(R^{4a}R^{4b}R^{4c});
R^{4a} is halo-C₁-C₆-alkyl; R^{4b} is hydroxy; and R^{4c} is hydrogen; or
R^{4a}, R^{4b}, and R^{4c} are each independently halogen; or
R^{4a} and R^{4b}, taken together with the carbon atom to which they are attached, form a C₃-C₁₀-cycloalkyl or a 3- to 14-membered heterocyclyl; and R^{4c} is selected from hydrogen, hydroxy, amino, and halogen; wherein said C₃-C₁₀-cycloalkyl and 3- to 14-membered heterocyclyl are optionally substituted with 1-2 halogen substituents;
R⁵ is selected from hydrogen, hydroxy, halogen, cyano, C₁-C₆-alkyl, C₁-C₆-alkoxy, halo-C₁-C₆-alkyl, hydroxy-C₁-C₆-alkyl, hydroxy-halo-C₁-C₆-alkyl, carboxy-C₁-C₆-alkyl, halo-C₁-C₆-alkoxy, oxo, NH₂—SO₂—, C₁-C₆-alkyl-NH—, carbamoyl, C₁-C₆-alkyl-NH—C(O)—, carboxy, C₁-C₆-alkoxycarbonyl, C₁-C₆-alkoxycarbonyl-C₁-C₆-alkyl-, (C₁-C₆-alkyl)₂P(O)—, (C₁-C₆-alkyl)₂P(O)—C₁-C₆-alkyl-, a group 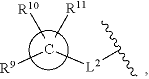
and a group 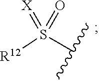
R⁶ is selected from hydrogen, halogen, cyano, oxo, C₁-C₆-alkoxy, halo-C₁-C₆-alkoxy, halo-C₁-C₆-alkyl, and C₁-C₆-alkyl-SO₂—;
R⁷ is selected from hydrogen and C₁-C₆-alkyl;
R^{8a} is selected from hydrogen, halogen, C₁-C₆-alkyl, and carbamoyl;
R^{8b} is selected from hydrogen and halogen;
R⁹ is selected from hydrogen, halogen, cyano, hydroxy, oxo, carboxy, C₁-C₆-alkoxycarbonyl, C₁-C₆-alkyl-NH—, carbamoyl, C₁-C₆-alkyl-NH—C(O)—, C₁-C₆-alkyl, hydroxy-C₁-C₆-alkyl, halo-C₁-C₆-alkyl, C₁-C₆-alkoxy-C₁-C₆-alkyl, C₁-C₆-alkoxy, halo-C₁-C₆-alkoxy, C₃-C₁₀-cycloalkyl, cyano, C₁-C₆-alkyl-SO₂—, halo-C₁-C₆-alkyl-SO₂—, and 5- to 14-membered heteroaryl; wherein said C₃-C₁₀-cycloalkyl and 5- to 14-membered heteroaryl is optionally substituted with one substituent selected from C₁-C₆-alkyl and halo-C₁-C₆-alkyl; or
R⁹ and R⁵ together form a group 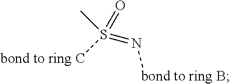
R¹⁰ is selected from hydrogen, halogen, C₁-C₆-alkyl, halo-C₁-C₆-alkyl, and oxo;
R¹¹ is selected from hydrogen and halogen;
R¹² is selected from amino, C₁-C₆-alkyl and halo-C₁-C₆-alkyl;
R^{13a} is selected from hydrogen, C₁-C₆-alkyl, C₃-C₁₀-cycloalkyl, C₃-C₁₀-cycloalkyl-C₁-C₆-alkyl-;
R^{13b} is hydrogen; and
R^{13c} is selected from hydrogen and C₁-C₆-alkyl.

11. The compound of formula (I) according to claim 10, or a pharmaceutically acceptable salt thereof, wherein:
A is selected from: 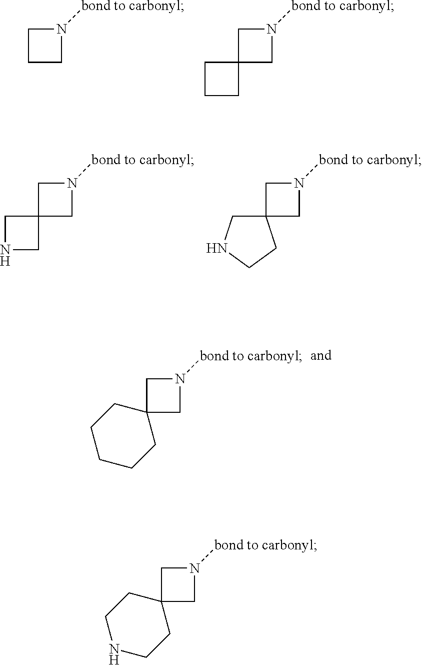
B is selected from C₆-C₁₄-aryl, C₃-C₁₀-cycloalkyl, 5- to 14-membered heteroaryl, and 3- to 14-membered heterocyclyl;
C is selected from C₃-C₁₀-cycloalkyl and 5- to 14-membered heteroaryl;
L¹ is selected from a covalent bond, —CR^{8a}R^{8b}—, and —CH₂O—;
L² is selected from a covalent bond, —NH—, and —CH₂NH—;
X is NH or O;
R¹ is a group 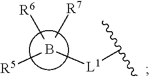
R² is hydrogen;
R³ is hydrogen;
R⁴ is a group —C(R^{4a}R^{4b}R^{4c});
R^{4a}, R^{4b}, and R^{4c} are each independently halogen; or
R^{4a} and R^{4b}, taken together with the carbon atom to which they are attached, form a C₃-C₁₀-cycloalkyl; and R^{4c} is selected from hydrogen, hydroxy, and halogen; wherein said C₃-C₁₀-cycloalkyl is optionally substituted with 1-2 halogen substituents;
R⁵ is selected from halogen, cyano, halo-C₁-C₆-alkyl, halo-C₁-C₆-alkoxy, C₁-C₆—alkyl-SO₂—, halo-C ₁—C₆-alkyl-SO₂—, oxo, a group 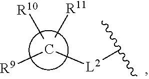
and a group 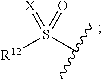
R⁶ is selected from hydrogen, halogen, cyano, and halo-C₁-C₆-alkyl;
R⁷ is selected from hydrogen and C₁-C₆-alkyl;
R^{8a} is selected from hydrogen and halogen;
R^{8b} is selected from hydrogen and halogen;
R⁹ is selected from hydrogen, C₁-C₆-alkyl and halo-C₁-C₆-alkyl;
R¹⁰ is selected from hydrogen, C₁-C₆-alkyl, and halo-C₁-C₆-alkyl;
R¹¹ is hydrogen; and
R¹² is halo-C₁-C₆-alkyl.

12. The compound of formula (I) according to claim 11, or a pharmaceutically acceptable salt thereof, wherein:
A is selected from: 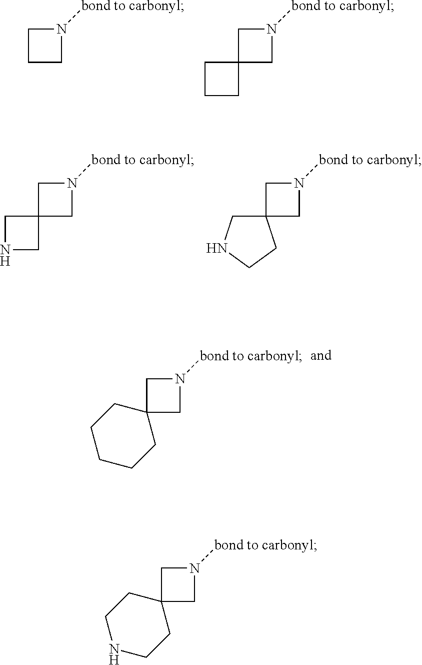
B is selected from phenyl, bicyclo[1.1.1]pentanyl, pyridyl, oxazolyl, pyrimidinyl, pyrazinyl, pyridazinyl, 1,2,4-thiadiazolyl, pyrazolyl, imidazolyl, 1H-1,2,4-triazolyl, triazol-2-yl, 2H-triazolyl, isoxazolyl, 1,2,4-oxadiazolyl, 1,3,4-oxadiazolyl, 1,2-dihydropyridyl, and 1,2-dihydropyrazinyl;
C is selected from cyclopropyl, pyrazolyl, 1,3,4-oxadiazole, pyrazinyl, and 1,3,4-thiadiazolyl;
L¹ is selected from a covalent bond, —CR^{8a}R^{8b}—, and —CH₂O—;
L² is selected from a covalent bond, —NH—, and —CH₂NH—;
X is NH or O;
R¹ is a group 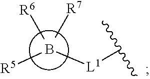
R² is hydrogen;
R³ is hydrogen;
R⁴ is a group —C(R^{4a}R^{4b}R^{4c});
R^{4a}, R^{4b}, and R^{4c} are each independently fluoro; or
R^{4a} and R^{4b}, taken together with the carbon atom to which they are attached, form a cyclopropyl; and R^{4c} is selected from hydrogen, hydroxy, and fluoro; wherein said cyclopropyl is optionally substituted with 1-2 fluoro substituents;
R⁵ is selected from chloro, fluoro, cyano, CF₃, 2,2,2-trifluoroethyl, CF₃O, methylsulfonyl, CF₃—SO₂—, oxo, a group 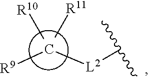
and a group 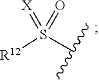
R⁶ is selected from hydrogen, fluoro, cyano, and CF₃;
R⁷ is selected from hydrogen and methyl;
R^{8a} is selected from hydrogen and fluoro;
R^{8b} is selected from hydrogen and fluoro;
R⁹ is selected from hydrogen, methyl, 2,2-dimethylpropyl, CHF₂ and CF₃;
R¹⁰ is selected from hydrogen, methyl, and CF₃;
R¹¹ is hydrogen; and
R¹² is CF₃.

13. The compound of formula (I) according to claim 1, or a pharmaceutically acceptable salt thereof, selected from:
[6-(5-cyclopropyl-4H-1,2,4-triazol-3-yl)-2-azaspiro[3.3]heptan-2-yl]-[3-[6-[[1-(trifluoromethyl)cyclopropyl]amino]-3-pyridyl]azetidin-1-yl]methanone;
[6-(3-cyclopropyl-1H-1,2,4-triazol-5-yl)-2-azaspiro[3.3]heptan-2-yl]-[6-[4-(trifluoromethylsulfonimidoyl)benzyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[5-(trifluoromethyl)pyrazin-2-yl]methyl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[[5-(trifluoromethyl)pyrazin-2-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[6-(trifluoromethyl)-3-pyridyl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[6-(trifluoromethyl)-3-pyridyl]methyl]-2-azaspiro[3.3]heptan-2-yl]methanone;
[6-(5-cyclobutyl-4H-1,2,4-triazol-3-yl)-2-azaspiro[3.3]heptan-2-yl]-[6-[[6-(trifluoromethyl)-3-pyridyl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[5-(trifluoromethoxy)-2-pyridyl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[5-(trifluoromethoxy)-2-pyridyl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[3-fluoro-5-(trifluoromethyl)-2-pyridyl]methyl]-2-azaspiro[3.3]heptan-2-yl]methanone;
[6-[[3-fluoro-5-(trifluoromethyl)-2-pyridyl]methyl]-2-azaspiro[3.3]heptan-2-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[6-[(4-methylsulfonylphenyl)methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[(4-methylsulfonylphenyl)methyl]-2-azaspiro[3.3]heptan-2-yl]methanone;
[6-(5-cyclobutyl-4H-1,2,4-triazol-3-yl)-2-azaspiro[3.3]heptan-2-yl]-[6-[(4-methylsulfonylphenyl)methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[(3-methylsulfonylphenyl)methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[(3-methylsulfonylphenyl)methyl]-2-azaspiro[3.3]heptan-2-yl]methanone;
[6-(5-cyclobutyl-4H-1,2,4-triazol-3-yl)-2-azaspiro[3.3]heptan-2-yl]-[6-[(3-methylsulfonylphenyl)methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[(3-fluoro-5-methylsulfonyl-phenyl)methyl]-2-azaspiro[3.3]heptan-2-yl]methanone;
[6-[(3-fluoro-5-methylsulfonyl-phenyl)methyl]-2-azaspiro[3.3]heptan-2-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[6-[[2-(trifluoromethyl)pyrimidin-5-yl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[2-(trifluoromethyl)pyrimidin-5-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[6-(trifluoromethyl)pyridazin-3-yl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[6-(trifluoromethyl)pyridazin-3-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[(4-fluoro-2-methylsulfonyl-phenyl)methyl]-2-azaspiro[3.3]heptan-2-yl]methanone;
[6-(5-cyclobutyl-4H-1,2,4-triazol-3-yl)-2-azaspiro[3.3]heptan-2-yl]-[6-[(4-fluoro-2-methylsulfonyl-phenyl)methyl]-2-azaspiro[3.3]heptan-2-yl]methanone;
5-[[2-[6-(5-cyclopropyl-4H-1,2,4-triazol-3-yl)-2-azaspiro[3.3]heptane-2-carbonyl]-2-azaspiro[3.3]heptan-6-yl]methyl]-2-(trifluoromethyl)benzonitrile;
5-[[2-[6-[5-(1-hydroxycyclopropyl)-4H-1,2,4-triazol-3-yl]-2-azaspiro[3.3]heptane-2-carbonyl]-2-azaspiro[3.3]heptan-6-yl]methyl]-2-(trifluoromethyl)benzonitrile;
[6-(5-cyclopropyl-4H-1,2,4-triazol-3-yl)-2-azaspiro[3.3]heptan-2-yl]-[6-[[5-(trifluoromethyl)pyrimidin-2-yl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[5-(trifluoromethyl)pyrimidin-2-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[3-(trifluoromethylsulfonyl)phenyl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[3-(trifluoromethylsulfonyl)phenyl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[4-fluoro-2-(trifluoromethyl)phenyl]methyl]-2-azaspiro[3.3]heptan-2-yl]methanone;
[6-[[4-fluoro-2-(trifluoromethyl)phenyl]methyl]-2-azaspiro[3.3]heptan-2-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[6-[(2,4-difluorophenyl)methyl]-2-azaspiro[3.3]heptan-2-yl]methanone;
[6-[(2,4-difluorophenyl)methyl]-2-azaspiro[3.3]heptan-2-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[6-[[2-fluoro-4-(trifluoromethyl)phenyl]methyl]-2-azaspiro[3.3]heptan-2-yl]methanone;
[6-[[2-fluoro-4-(trifluoromethyl)phenyl]methyl]-2-azaspiro[3.3]heptan-2-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[6-[[2-methylsulfonyl-4-(trifluoromethyl)phenyl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[2-methylsulfonyl-4-(trifluoromethyl)phenyl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[3-methylsulfonyl-4-(trifluoromethyl)phenyl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[3-methylsulfonyl-4-(trifluoromethyl)phenyl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[(5-fluoro-2-pyridyl)methyl]-2-azaspiro[3.3]heptan-2-yl]methanone;
[6-[(5-fluoro-2-pyridyl)methyl]-2-azaspiro[3.3]heptan-2-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[6-[(5-fluoro-3-pyridyl)methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[4-methylsulfonyl-3-(trifluoromethyl)phenyl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[4-methylsulfonyl-3-(trifluoromethyl)phenyl]methyl]-2-azaspiro[3.3]heptan-2-yl]methanone;
[6-(5-cyclopropyl-4H-1,2,4-triazol-3-yl)-2-azaspiro[3.3]heptan-2-yl]-[7-[[5-(trifluoromethyl)-2-pyridyl]methyl]-2-azaspiro[3.5]nonan-2-yl]methanone;
[6-[5-(1-hydroxycyclopropyl)-4H-1,2,4-triazol-3-yl]-2-azaspiro[3.3]heptan-2-yl]-[7-[[5-(trifluoromethyl)-2-pyridyl]methyl]-2-azaspiro[3.5]nonan-2-yl]methanone;
[6-(5-cyclopropyl-4H-1,2,4-triazol-3-yl)-2-azaspiro[3.3]heptan-2-yl]-[6-[[5-(trifluoromethyl)-2-pyridyl]methyl]-2-azaspiro[3.4]octan-2-yl]methanone;
[6-(5-cyclopropyl-4H-1,2,4-triazol-3-yl)-2-azaspiro[3.3]heptan-2-yl]-[7-[6-(trifluoromethyl)pyridazin-3-yl]oxy-2-azaspiro[3.5]nonan-2-yl]methanone;
[6-(5-cyclobutyl-4H-1,2,4-triazol-3-yl)-2-azaspiro[3.3]heptan-2-yl]-[7-[6-(trifluoromethyl)pyridazin-3-yl]oxy-2-azaspiro[3.5]nonan-2-yl]methanone;
[6-(5-cyclopropyl-4H-1,2,4-triazol-3-yl)-2-azaspiro[3.3]heptan-2-yl]-[3-[6-(4-methyl-4-oxo-1,4λ⁵-azaphosphinan-1-yl)-3-pyridyl]azetidin-1-yl]methanone;
[6-(5-cyclopropyl-4H-1,2,4-triazol-3-yl)-2-azaspiro[3.3]heptan-2-yl]-[6-[[[1-(trifluoromethyl)cyclopropyl]amino]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[[1-(trifluoromethyl)cyclopropyl]amino]methyl]-2-azaspiro[3.3]heptan-2-yl]methanone;
N-[2-[6-(5-cyclopropyl-4H-1,2,4-triazol-3-yl)-2-azaspiro[3.3]heptane-2-carbonyl]-2-azaspiro[3.3]heptan-6-yl]-3-(trifluoromethyl)benzenesulfonamide;
[6-(5-cyclopropyl-4H-1,2,4-triazol-3-yl)-2-azaspiro[3.3]heptan-2-yl]-[6-[[6-(trifluoromethyl)pyridazin-3-yl]amino]-2-azaspiro[3.3]heptan-2-yl]methanone;
[6-(5-cyclopropyl-4H-1,2,4-triazol-3-yl)-2-azaspiro[3.3]heptan-2-yl]-[6-[[4-(trifluoromethyl)pyrazol-1-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[3-(trifluoromethyl)-1,2,4-thiadiazol-5-yl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[3-(trifluoromethyl)-1,2,4-thiadiazol-5-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[4-(trifluoromethyl)oxazol-2-yl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[4-(trifluoromethyl)oxazol-2-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[4-(trifluoromethyl)imidazol-1-yl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[4-(trifluoromethyl)imidazol-1-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[7-[[1-(trifluoromethyl)cyclopropyl]methoxy]-2-azaspiro[3.5]nonan-2-yl]methanone;
[6-[5-(1-hydroxycyclopropyl)-4H-1,2,4-triazol-3-yl]-2-azaspiro[3.3]heptan-2-yl]-[7-[[1-(trifluoromethyl)cyclopropyl]methoxy]-2-azaspiro[3.5]nonan-2-yl]methanone;
[6-(5-cyclopropyl-4H-1,2,4-triazol-3-yl)-2-azaspiro[3.3]heptan-2-yl]-[6-[5-(trifluoromethyl)pyrazin-2-yl]-2-azaspiro[3.3]heptan-2-yl]methanone;
[6-(5-cyclopropyl-4H-1,2,4-triazol-3-yl)-2-azaspiro[3.3]heptan-2-yl]-[6-[[1-(trifluoromethyl)cyclopropyl]methoxy]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[1-(trifluoromethyl)cyclopropyl]methoxy]-2-azaspiro[3.3]heptan-2-yl]methanone;
[6-(5-cyclopropyl-4H-1,2,4-triazol-3-yl)-2-azaspiro[3.3]heptan-2-yl]-[6-[[1-(trifluoromethyl)cyclopropyl]amino]-2-azaspiro[3.3]heptan-2-yl]methanone;
[6-(5-cyclopropyl-4H-1,2,4-triazol-3-yl)-2-azaspiro[3.3]heptan-2-yl]-[6-[[3-(trifluoromethyl)-1,2,4-triazol-1-yl]methyl]-2-azaspiro[3.3]heptan-2-yl]methanone;
2-[6-(5-cyclopropyl-4H-1,2,4-triazol-3-yl)-2-azaspiro[3.3]heptane-2-carbonyl]-N-[1-(trifluoromethyl)cyclopropyl]-2-azaspiro[3.3]heptane-6-carboxamide;
[6-(5-cyclopropyl-4H-1,2,4-triazol-3-yl)-2-azaspiro[3.3]heptan-2-yl]-[7-[2-(trifluoromethyl)pyrimidin-4-yl]oxy-2-azaspiro[3.5]nonan-2-yl]methanone;
[6-(5-cyclopropyl-4H-1,2,4-triazol-3-yl)-2-azaspiro[3.3]heptan-2-yl]-[7-[5-(trifluoromethyl)pyrazin-2-yl]oxy-2-azaspiro[3.5]nonan-2-yl]methanone;
[6-(5-cyclopropyl-4H-1,2,4-triazol-3-yl)-2-azaspiro[3.3]heptan-2-yl]-[6-[5-(trifluoromethyl)pyrazin-2-yl]oxy-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[5-(trifluoromethyl)pyrazin-2-yl]oxy-2-azaspiro[3.3]heptan-2-yl]methanone;
[6-(5-cyclopropyl-4H-1,2,4-triazol-3-yl)-2-azaspiro[3.3]heptan-2-yl]-[6-[(4-methylsulfonylphenyl)methyl]-2-azaspiro[3.4]octan-2-yl]methanone;
[6-(5-cyclopropyl-4H-1,2,4-triazol-3-yl)-2-azaspiro[3.3]heptan-2-yl]-[6-[6-(trifluoromethyl)pyridazin-3-yl]oxy-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[6-(trifluoromethyl)pyridazin-3-yl]oxy-2-azaspiro[3.3]heptan-2-yl]methanone;
[6-(5-cyclopropyl-4H-1,2,4-triazol-3-yl)-2-azaspiro[3.3]heptan-2-yl]-[3-[6-(1,1-dioxo-1,4-thiazinan-4-yl)-3-pyridyl]azetidin-1-yl]methanone;
[6-(5-cyclopropyl-4H-1,2,4-triazol-3-yl)-2-azaspiro[3.3]heptan-2-yl]-[3-[6-[(1,1-dioxothiolan-3-yl)amino]-3-pyridyl]azetidin-1-yl]methanone;
[6-(5-cyclopropyl-4H-1,2,4-triazol-3-yl)-2-azaspiro[3.3]heptan-2-yl]-[3-[6-(3-hydroxy-3-methyl-azetidin-1-yl)-3-pyridyl]azetidin-1-yl]methanone;
[6-(5-cyclopropyl-4H-1,2,4-triazol-3-yl)-2-azaspiro[3.3]heptan-2-yl]-[6-[3-(trifluoromethyl)azetidin-1-yl]-2-azaspiro[3.3]heptan-2-yl]methanone;
[6-(5-cyclopropyl-4H-1,2,4-triazol-3-yl)-2-azaspiro[3.3]heptan-2-yl]-[6-[3-(2,2,2-trifluoroethyl)azetidin-1-yl]-2-azaspiro[3.3]heptan-2-yl]methanone;
[6-(5-cyclopropyl-4H-1,2,4-triazol-3-yl)-2-azaspiro[3.3]heptan-2-yl]-[7-[[4-(trifluoromethylsulfonyl)phenyl]methyl]-2,7-diazaspiro[3.5]nonan-2-yl]methanone;
[6-(5-cyclopropyl-4H-1,2,4-triazol-3-yl)-2-azaspiro[3.3]heptan-2-yl]-[6-[[4-(trifluoromethylsulfonyl)phenyl]methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[2-[3-(trifluoromethoxy)phenyl]sulfonyl-2,6-diazaspiro[3.3]heptan-6-yl]methanone;
[6-[5-(1-hydroxycyclopropyl)-4H-1,2,4-triazol-3-yl]-2-azaspiro[3.3]heptan-2-yl]-[2-[3-(trifluoromethoxy)phenyl]sulfonyl-2,6-diazaspiro[3.3]heptan-6-yl]methanone;
[6-(5-cyclobutyl-4H-1,2,4-triazol-3-yl)-2-azaspiro[3.3]heptan-2-yl]-[2-[3-(trifluoromethoxy)phenyl]sulfonyl-2,6-diazaspiro[3.3]heptan-6-yl]methanone;
[6-(5-cyclopropyl-4H-1,2,4-triazol-3-yl)-2-azaspiro[3.3]heptan-2-yl]-[(6S)-6-[5-(trifluoromethyl)pyrazin-2-yl]oxy-2-azaspiro[3.4]octan-2-yl]methanone;
[6-(5-cyclopropyl-4H-1,2,4-triazol-3-yl)-2-azaspiro[3.3]heptan-2-yl]-[2-[4-(trifluoromethoxy)phenyl]sulfonyl-2,6-diazaspiro[3.3]heptan-6-yl]methanone;
[6-[5-(1-hydroxycyclopropyl)-4H-1,2,4-triazol-3-yl]-2-azaspiro[3.3]heptan-2-yl]-[2-[4-(trifluoromethoxy)phenyl]sulfonyl-2,6-diazaspiro[3.3]heptan-6-yl]methanone;
[6-(5-cyclobutyl-4H-1,2,4-triazol-3-yl)-2-azaspiro[3.3]heptan-2-yl]-[2-[4-(trifluoromethoxy)phenyl]sulfonyl-2,6-diazaspiro[3.3]heptan-6-yl]methanone;
[6-(5-cyclopropyl-4H-1,2,4-triazol-3-yl)-2-azaspiro[3.3]heptan-2-yl]-[2-[2-(trifluoromethoxy)phenyl]sulfonyl-2,6-diazaspiro[3.3]heptan-6-yl]methanone;
[6-[5-(1-hydroxycyclopropyl)-4H-1,2,4-triazol-3-yl]-2-azaspiro[3.3]heptan-2-yl]-[2-[2-(trifluoromethoxy)phenyl]sulfonyl-2,6-diazaspiro[3.3]heptan-6-yl]methanone;
[6-(5-cyclopropyl-4H-1,2,4-triazol-3-yl)-2-azaspiro[3.3]heptan-2-yl]-[2-[2-(trifluoromethyl)phenyl]sulfonyl-2,6-diazaspiro[3.3]heptan-6-yl]methanone;
[6-[5-(1-hydroxycyclopropyl)-4H-1,2,4-triazol-3-yl]-2-azaspiro[3.3]heptan-2-yl]-[2-[2-(trifluoromethyl)phenyl]sulfonyl-2,6-diazaspiro[3.3]heptan-6-yl]methanone;
[6-(5-cyclopropyl-4H-1,2,4-triazol-3-yl)-2-azaspiro[3.3]heptan-2-yl]-[2-[4-fluoro-2-(trifluoromethyl)phenyl]sulfonyl-2,6-diazaspiro[3.3]heptan-6-yl]methanone;
[6-(5-cyclopropyl-4H-1,2,4-triazol-3-yl)-2-azaspiro[3.3]heptan-2-yl]-[2-[3-(trifluoromethyl)phenyl]sulfonyl-2,6-diazaspiro[3.3]heptan-6-yl]methanone;
[6-[5-(1-hydroxycyclopropyl)-4H-1,2,4-triazol-3-yl]-2-azaspiro[3.3]heptan-2-yl]-[2-[3-(trifluoromethyl)phenyl]sulfonyl-2,6-diazaspiro[3.3]heptan-6-yl]methanone;
[6-(5-cyclopropyl-4H-1,2,4-triazol-3-yl)-2-azaspiro[3.3]heptan-2-yl]-[2-[3-fluoro-5-(trifluoromethyl)phenyl]sulfonyl-2,6-diazaspiro[3.3]heptan-6-yl]methanone;
[6-(5-cyclopropyl-4H-1,2,4-triazol-3-yl)-2-azaspiro[3.3]heptan-2-yl]-[2-[4-fluoro-3-(trifluoromethyl)phenyl]sulfonyl-2,6-diazaspiro[3.3]heptan-6-yl]methanone;
[6-(5-cyclopropyl-4H-1,2,4-triazol-3-yl)-2-azaspiro[3.3]heptan-2-yl]-[2-[4-(trifluoromethyl)phenyl]sulfonyl-2,6-diazaspiro[3.3]heptan-6-yl]methanone;
[6-[5-(1-hydroxycyclopropyl)-4H-1,2,4-triazol-3-yl]-2-azaspiro[3.3]heptan-2-yl]-[2-[4-(trifluoromethyl)phenyl]sulfonyl-2,6-diazaspiro[3.3]heptan-6-yl]methanone;
[6-(5-cyclopropyl-4H-1,2,4-triazol-3-yl)-2-azaspiro[3.3]heptan-2-yl]-[2-(2,4-difluorophenyl)sulfonyl-2,6-diazaspiro[3.3]heptan-6-yl]methanone;
[6-(5-cyclopropyl-4H-1,2,4-triazol-3-yl)-2-azaspiro[3.3]heptan-2-yl]-[2-(3,4-difluorophenyl)sulfonyl-2,6-diazaspiro[3.3]heptan-6-yl]methanone;
[2-(2-chloro-4-fluoro-phenyl)sulfonyl-2,6-diazaspiro[3.3]heptan-6-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[2-(2-methoxyphenyl)sulfonyl-2,6-diazaspiro[3.3]heptan-6-yl]methanone;
[6-(5-cyclopropyl-4H-1,2,4-triazol-3-yl)-2-azaspiro[3.3]heptan-2-yl]-[2-[[5-(trifluoromethyl)-3-pyridyl]sulfonyl]-2,6-diazaspiro[3.3]heptan-6-yl]methanone;
[6-(5-cyclopropyl-4H-1,2,4-triazol-3-yl)-2-azaspiro[3.3]heptan-2-yl]-[6-[[5-(trifluoromethyl)-2-pyridyl]methyl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[5-(trifluoromethyl)-2-pyridyl]methyl]-2-azaspiro[3.3]heptan-2-yl]methanone;
[6-(5-cyclobutyl-4H-1,2,4-triazol-3-yl)-2-azaspiro[3.3]heptan-2-yl]-[6-[[5-(trifluoromethyl)-2-pyridyl]methyl]-2-azaspiro[3.3]heptan-2-yl]methanone;
[6-[(5-chloro-3-pyridyl)methyl]-2-azaspiro[3.3]heptan-2-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[6-[[5-(trifluoromethyl)-3-pyridyl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[(3,5-difluoro-2-pyridyl)methyl]-2-azaspiro[3.3]heptan-2-yl]methanone;
[6-[(3,5-difluoro-2-pyridyl)methyl]-2-azaspiro[3.3]heptan-2-yl]-[6-[5-(1-hydroxycyclopropyl)-4H-1,2,4-triazol-3-yl]-2-azaspiro[3.3]heptan-2-yl]methanone;
[6-[(3-chloro-5-fluoro-2-pyridyl)methyl]-2-azaspiro[3.3]heptan-2-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[6-[[4-(trifluoromethyl)-2-pyridyl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[(5-methylsulfonyl-2-pyridyl)methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[(5-methylsulfonyl-3-pyridyl)methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[3-fluoro-5-(trifluoromethyl)phenyl]methyl]-2-azaspiro[3.3]heptan-2-yl]methanone;
[6-[[3-fluoro-5-(trifluoromethyl)phenyl]methyl]-2-azaspiro[3.3]heptan-2-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[6-[[4-(methylsulfonimidoyl)phenyl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[3-(methylsulfonimidoyl)phenyl]methyl]-2-azaspiro[3.3]heptan-2-yl]methanone;
(rac)-[6-(5-cyclopropyl-4H-1,2,4-triazol-3-yl)-2-azaspiro[3.3]heptan-2-yl]-[6-[[3-(trifluoromethylsulfonimidoyl)phenyl]methyl]-2-azaspiro[3.3]heptan-2-yl]methanone;
(S)-[6-(5-cyclopropyl-4H-1,2,4-triazol-3-yl)-2-azaspiro[3.3]heptan-2-yl]-[6-[[3-(trifluoromethylsulfonimidoyl)phenyl]methyl]-2-azaspiro[3.3]heptan-2-yl]methanone;
(R)-[6-(5-cyclopropyl-4H-1,2,4-triazol-3-yl)-2-azaspiro[3.3]heptan-2-yl]-[6-[[3-(trifluoromethylsulfonimidoyl)phenyl]methyl]-2-azaspiro[3.3]heptan-2-yl]methanone;
(R)-[6-(5-cyclopropyl-4H-1,2,4-triazol-3-yl)-2-azaspiro[3.3]heptan-2-yl]-[6-[[4-(trifluoromethylsulfonimidoyl)phenyl]methyl]-2-azaspiro[3.3]heptan-2-yl]methanone;
(S)-[6-(5-cyclopropyl-4H-1,2,4-triazol-3-yl)-2-azaspiro[3.3]heptan-2-yl]-[6-[[4-(trifluoromethylsulfonimidoyl)phenyl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[(2-fluoro-4-methylsulfonyl-phenyl)methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[6-(trifluoromethyl)-3-pyridyl]oxy]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[6-[[6-(trifluoromethyl)-3-pyridyl]oxy]-2-azaspiro[3.3]heptan-2-yl]methanone;
[6-(5-cyclopropyl-4H-1,2,4-triazol-3-yl)-2-azaspiro[3.3]heptan-2-yl]-[6-[6-(trifluoromethyl)pyrazin-2-yl]oxy-2-azaspiro[3.3]heptan-2-yl]methanone;
[6-(5-cyclopropyl-4H-1,2,4-triazol-3-yl)-2-azaspiro[3.3]heptan-2-yl]-[7-[5-(trifluoromethyl)pyrimidin-2-yl]oxy-2-azaspiro[3.5]nonan-2-yl]methanone;
[6-(5-cyclopropyl-4H-1,2,4-triazol-3-yl)-2-azaspiro[3.3]heptan-2-yl]-[2-[[3-(trifluoromethyl)-1-bicyclo[1.1.1]pentanyl]sulfonyl]-2,6-diazaspiro[3.3]heptan-6-yl]methanone;
[6-(5-cyclopropyl-4H-1,2,4-triazol-3-yl)-2-azaspiro[3.3]heptan-2-yl]-[7-[[5-(trifluoromethyl)pyrazin-2-yl]methyl]-2-azaspiro[3.5]nonan-2-yl]methanone;
[6-[5-(1-hydroxycyclopropyl)-4H-1,2,4-triazol-3-yl]-2-azaspiro[3.3]heptan-2-yl]-[7-[[5-(trifluoromethyl)pyrazin-2-yl]methyl]-2-azaspiro[3.5]nonan-2-yl]methanone;
[7-[(5-chloro-2-pyridyl)methyl]-2-azaspiro[3.5]nonan-2-yl]-[6-(5-cyclopropyl-4H-1,2,4-triazol-3-yl)-2-azaspiro[3.3]heptan-2-yl]methanone;
[7-[(5-chloro-2-pyridyl)methyl]-2-azaspiro[3.5]nonan-2-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[7-[[6-(trifluoromethyl)-3-pyridyl]methyl]-2-azaspiro[3.5]nonan-2-yl]methanone;
[6-[5-(1-hydroxycyclopropyl)-4H-1,2,4-triazol-3-yl]-2-azaspiro[3.3]heptan-2-yl]-[7-[[6-(trifluoromethyl)-3-pyridyl]methyl]-2-azaspiro[3.5]nonan-2-yl]methanone;
[6-(5-cyclopropyl-4H-1,2,4-triazol-3-yl)-2-azaspiro[3.3]heptan-2-yl]-[7-[(3,5-difluoro-2-pyridyl)methyl]-2-azaspiro[3.5]nonan-2-yl]methanone;
[7-[(3,5-difluoro-2-pyridyl)methyl]-2-azaspiro[3.5]nonan-2-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[7-[(5-fluoro-2-pyridyl)methyl]-2-azaspiro[3.5]nonan-2-yl]methanone;
[7-[(5-fluoro-2-pyridyl)methyl]-2-azaspiro[3.5]nonan-2-yl]-[6-[5-(1-hydroxycyclopropyl)-4H-1,2,4-triazol-3-yl]-2-azaspiro[3.3]heptan-2-yl]methanone;
[7-[[6-(difluoromethoxy)-3-pyridyl]methyl]-2-azaspiro[3.5]nonan-2-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[6-[(3,5-difluoro-2-pyridyl)methyl]-2-azaspiro[3.4]octan-2-yl]methanone;
[6-(5-cyclopropyl-4H-1,2,4-triazol-3-yl)-2-azaspiro[3.3]heptan-2-yl]-[rac-(6S)-6-[(3,5-difluoro-2-pyridyl)methyl]-2-azaspiro[3.4]octan-2-yl]methanone;
[6-(5-cyclopropyl-4H-1,2,4-triazol-3-yl)-2-azaspiro[3.3]heptan-2-yl]-[rac-(6R)-6-[(3,5-difluoro-2-pyridyl)methyl]-2-azaspiro[3.4]octan-2-yl]methanone;
[6-(5-cyclopropyl-4H-1,2,4-triazol-3-yl)-2-azaspiro[3.3]heptan-2-yl]-[rac-(6R)-6-[[5-(trifluoromethyl)pyrazin-2-yl]methyl]-2-azaspiro[3.4]octan-2-yl]methanone;
[6-(5-cyclopropyl-4H-1,2,4-triazol-3-yl)-2-azaspiro[3.3]heptan-2-yl]-[rac-(6S)-6-[[5-(trifluoromethyl)pyrazin-2-yl]methyl]-2-azaspiro[3.4]octan-2-yl]methanone;
[6-(5-cyclopropyl-4H-1,2,4-triazol-3-yl)-2-azaspiro[3.3]heptan-2-yl]-[6-[[6-(trifluoromethyl)-3-pyridyl]methyl]-2-azaspiro[3.4]octan-2-yl]methanone;
[6-(5-cyclopropyl-4H-1,2,4-triazol-3-yl)-2-azaspiro[3.3]heptan-2-yl]-[6-[[4-methylsulfonyl-3-(trifluoromethyl)phenyl]methyl]-2-azaspiro[3.4]octan-2-yl]methanone;
5-[[rac-(6S)-2-[6-(5-cyclopropyl-4H-1,2,4-triazol-3-yl)-2-azaspiro[3.3]heptane-2-carbonyl]-2-azaspiro[3.4]octan-6-yl]oxy]-2-(trifluoromethyl)pyridine-4-carbonitrile;
[6-(5-cyclopropyl-4H-1,2,4-triazol-3-yl)-2-azaspiro[3.3]heptan-2-yl]-[rac-(6S)-6-[4-methylsulfonyl-3-(trifluoromethyl)phenoxy]-2-azaspiro[3.4]octan-2-yl]methanone;
[6-(5-cyclopropyl-4H-1,2,4-triazol-3-yl)-2-azaspiro[3.3]heptan-2-yl]-[6-[(2,4-difluorophenyl)methyl]-2,6-diazaspiro[3.3]heptan-2-yl]methanone;
[6-[(2,4-difluorophenyl)methyl]-2,6-diazaspiro[3.3]heptan-2-yl]-[6-[5-(1-hydroxycyclopropyl)-4H-1,2,4-triazol-3-yl]-2-azaspiro[3.3]heptan-2-yl]methanone;
[6-(5-cyclobutyl-4H-1,2,4-triazol-3-yl)-2-azaspiro[3.3]heptan-2-yl]-[6-[(2,4-difluorophenyl)methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[(3,5-difluorophenyl)methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[[6-(trifluoromethyl)pyridazin-3-yl]methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[[5-(trifluoromethyl)pyrimidin-2-yl]methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[[2-(trifluoromethyl)pyrimidin-5-yl]methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[[5-(trifluoromethyl)-2-pyridyl]methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[[5-(trifluoromethyl)pyrazin-2-yl]methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[(3,5-difluoro-2-pyridyl)methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[(4-methylsulfonylphenyl)methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[(3-methylsulfonylphenyl)methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[(2-methylsulfonylphenyl)methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[[4-fluoro-2-(trifluoromethyl)phenyl]methyl]-2,6-diazaspiro[3.3]heptan-2-yl]methanone;
2-[[2-[6-(5-cyclopropyl-4H-1,2,4-triazol-3-yl)-2-azaspiro[3.3]heptane-2-carbonyl]-2,6-diazaspiro[3.3]heptan-6-yl]methyl]benzenesulfonamide;
methyl 2-[[2-[6-(5-cyclopropyl-4H-1,2,4-triazol-3-yl)-2-azaspiro[3.3]heptane-2-carbonyl]-2,6-diazaspiro[3.3]heptan-6-yl]methyl]benzoate;
2-[[2-[6-(5-cyclopropyl-4H-1,2,4-triazol-3-yl)-2-azaspiro[3.3]heptane-2-carbonyl]-2,6-diazaspiro[3.3]heptan-6-yl]methyl]benzoic acid;
2-[[2-[6-(5-cyclopropyl-4H-1,2,4-triazol-3-yl)-2-azaspiro[3.3]heptane-2-carbonyl]-2,6-diazaspiro[3.3]heptan-6-yl]methyl]-N-methyl-benzamide;
[6-(5-cyclopropyl-4H-1,2,4-triazol-3-yl)-2-azaspiro[3.3]heptan-2-yl]-[6-[[2-[1-(methoxymethyl)-1,2,4-triazol-3-yl]phenyl]methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[[2-[1-(methoxymethyl)imidazol-2-yl]phenyl]methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[7-[[6-(trifluoromethyl)-3-pyridyl]methyl]-2,7-diazaspiro[3.5]nonan-2-yl]methanone;
[6-(5-cyclopropyl-4H-1,2,4-triazol-3-yl)-2-azaspiro[3.3]heptan-2-yl]-[7-[[2-fluoro-4-(trifluoromethyl)phenyl]methyl]-2,7-diazaspiro[3.5]nonan-2-yl]methanone;
[6-(5-cyclopropyl-4H-1,2,4-triazol-3-yl)-2-azaspiro[3.3]heptan-2-yl]-[7-[[5-(trifluoromethyl)-2-pyridyl]methyl]-2,7-diazaspiro[3.5]nonan-2-yl]methanone;
[6-(5-cyclopropyl-4H-1,2,4-triazol-3-yl)-2-azaspiro[3.3]heptan-2-yl]-[7-[(4-methylsulfonylphenyl)methyl]-2,7-diazaspiro[3.5]nonan-2-yl]methanone;
[6-(5-cyclopropyl-4H-1,2,4-triazol-3-yl)-2-azaspiro[3.3]heptan-2-yl]-[7-[[3-(trifluoromethylsulfonyl)phenyl]methyl]-2,7-diazaspiro[3.5]nonan-2-yl]methanone;
[6-(5-cyclopropyl-4H-1,2,4-triazol-3-yl)-2-azaspiro[3.3]heptan-2-yl]-[7-[(3-methylsulfonylphenyl)methyl]-2,7-diazaspiro[3.5]nonan-2-yl]methanone;
[6-(5-cyclopropyl-4H-1,2,4-triazol-3-yl)-2-azaspiro[3.3]heptan-2-yl]-[7-[[5-(trifluoromethyl)-2-pyridyl]methyl]-2,7-diazaspiro[3.4]octan-2-yl]methanone;
[6-(5-cyclopropyl-4H-1,2,4-triazol-3-yl)-2-azaspiro[3.3]heptan-2-yl]-[7-[[6-(trifluoromethyl)pyridazin-3-yl]methyl]-2,7-diazaspiro[3.4]octan-2-yl]methanone;
[6-(5-cyclopropyl-4H-1,2,4-triazol-3-yl)-2-azaspiro[3.3]heptan-2-yl]-[7-[[5-(trifluoromethyl)pyrazin-2-yl]methyl]-2,7-diazaspiro[3.4]octan-2-yl]methanone;
[6-(5-cyclopropyl-4H-1,2,4-triazol-3-yl)-2-azaspiro[3.3]heptan-2-yl]-[6-[[2-fluoro-4-(trifluoromethyl)phenyl]methylamino]-2-azaspiro[3.3]heptan-2-yl]methanone;
[6-(5-cyclopropyl-4H-1,2,4-triazol-3-yl)-2-azaspiro[3.3]heptan-2-yl]-[3-[[4-(trifluoromethyl)phenyl]methylamino]azetidin-1-yl]methanone;
[6-(5-cyclopropyl-4H-1,2,4-triazol-3-yl)-2-azaspiro[3.3]heptan-2-yl]-[3-[[5-fluoro-2-(trifluoromethyl)phenyl]methylamino]azetidin-1-yl]methanone;
[3-[[2-chloro-5-(trifluoromethyl)phenyl]methylamino]azetidin-1-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[3-[[2-fluoro-4-(trifluoromethoxy)phenyl]methylamino]azetidin-1-yl]methanone;
[3-[[2-chloro-4-(trifluoromethyl)phenyl]methylamino]azetidin-1-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[3-[[4-(trifluoromethoxy)phenyl]methylamino]azetidin-1-yl]methanone;
[6-(5-cyclopropyl-4H-1,2,4-triazol-3-yl)-2-azaspiro[3.3]heptan-2-yl]-[3-[[2-fluoro-4-(trifluoromethyl)phenyl]methylamino]azetidin-1-yl]methanone;
[6-(5-cyclopropyl-4H-1,2,4-triazol-3-yl)-2-azaspiro[3.3]heptan-2-yl]-[3-[[3-fluoro-4-(trifluoromethyl)phenyl]methylamino]azetidin-1-yl]methanone;
[3-[[3-chloro-5-(trifluoromethyl)phenyl]methylamino]azetidin-1-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[3-[[2-fluoro-5-(trifluoromethyl)phenyl]methylamino]azetidin-1-yl]methanone;
[6-(5-cyclopropyl-4H-1,2,4-triazol-3-yl)-2-azaspiro[3.3]heptan-2-yl]-[3-[[4-fluoro-2-(trifluoromethyl)phenyl]methylamino]azetidin-1-yl]methanone;
[6-(5-cyclopropyl-4H-1,2,4-triazol-3-yl)-2-azaspiro[3.3]heptan-2-yl]-[3-[[2-methoxy-4-(trifluoromethyl)phenyl]methylamino]azetidin-1-yl]methanone;
[3-[[2-fluoro-4-(trifluoromethyl)phenyl]methylamino]azetidin-1-yl]-[6-[5-(1-hydroxycyclopropyl)-4H-1,2,4-triazol-3-yl]-2-azaspiro[3.3]heptan-2-yl]methanone;
[3-[[5-chloro-2-(trifluoromethyl)phenyl]methylamino]azetidin-1-yl]-[6-(5-cyclopropyl-4H-1,2,4-triazol-3-yl)-2-azaspiro[3.3]heptan-2-yl]methanone;
[3-[cyclopropyl-[[2-fluoro-4-(trifluoromethyl)phenyl]methyl]amino]azetidin-1-yl]-[6-(5-cyclopropyl-4H-1,2,4-triazol-3-yl)-2-azaspiro[3.3]heptan-2-yl]methanone;
[3-[cyclopropylmethyl-[[2-fluoro-4-(trifluoromethyl)phenyl]methyl]amino]azetidin-1-yl]-[6-(5-cyclopropyl-4H-1,2,4-triazol-3-yl)-2-azaspiro[3.3]heptan-2-yl]methanone;
N-[1-[6-(5-cyclopropyl-4H-1,2,4-triazol-3-yl)-2-azaspiro[3.3]heptane-2-carbonyl]azetidin-3-yl]-3-(trifluoromethyl)benzenesulfonamide;
N-[1-[6-(5-cyclopropyl-4H-1,2,4-triazol-3-yl)-2-azaspiro[3.3]heptane-2-carbonyl]azetidin-3-yl]-2-fluoro-4-(trifluoromethyl)benzenesulfonamide;
N-[1-[6-(5-cyclopropyl-4H-1,2,4-triazol-3-yl)-2-azaspiro[3.3]heptane-2-carbonyl]azetidin-3-yl]-4,4-difluoro-cyclohexanesulfonamide;
N-[1-[6-(5-cyclopropyl-4H-1,2,4-triazol-3-yl)-2-azaspiro[3.3]heptane-2-carbonyl]azetidin-3-yl]-3-(trifluoromethoxy)benzenesulfonamide;
[6-(5-cyclobutyl-4H-1,2,4-triazol-3-yl)-2-azaspiro[3.3]heptan-2-yl]-[3-[[2-fluoro-4-(trifluoromethyl)phenyl]methylamino]azetidin-1-yl]methanone;
[6-(5-cyclopropyl-4H-1,2,4-triazol-3-yl)-2-azaspiro[3.3]heptan-2-yl]-[3-[4-(2,2,2-trifluoroethoxy)pyrazol-1-yl]azetidin-1-yl]methanone;
N-[[1-[6-(5-cyclopropyl-4H-1,2,4-triazol-3-yl)-2-azaspiro[3.3]heptane-2-carbonyl]azetidin-3-yl]methyl]-3-(trifluoromethyl)benzenesulfonamide;
N-[[1-[6-(5-cyclopropyl-4H-1,2,4-triazol-3-yl)-2-azaspiro[3.3]heptane-2-carbonyl]azetidin-3-yl]methyl]-4-(trifluoromethyl)benzenesulfonamide;
N-[[1-[6-[5-(1-hydroxycyclopropyl)-4H-1,2,4-triazol-3-yl]-2-azaspiro[3.3]heptane-2-carbonyl]azetidin-3-yl]methyl]-4-(trifluoromethyl)benzenesulfonamide;
N-[[1-[6-[5-(1-hydroxycyclopropyl)-4H-1,2,4-triazol-3-yl]-2-azaspiro[3.3]heptane-2-carbonyl]azetidin-3-yl]methyl]-3-(trifluoromethyl)benzenesulfonamide;
[6-(5-cyclopropyl-4H-1,2,4-triazol-3-yl)-2-azaspiro[3.3]heptan-2-yl]-[2-(2,2-dimethylpropylsulfonyl)-2,6-diazaspiro[3.3]heptan-6-yl]methanone;
6-[6-(5-cyclopropyl-4H-1,2,4-triazol-3-yl)-2-azaspiro[3.3]heptane-2-carbonyl]-N-(1-methylcyclopropyl)-2,6-diazaspiro[3.3]heptane-2-sulfonamide;
6-[6-(5-cyclopropyl-4H-1,2,4-triazol-3-yl)-2-azaspiro[3.3]heptane-2-carbonyl]-N-[(4-fluorophenyl)methyl]-2,6-diazaspiro[3.3]heptane-2-sulfonamide;
6-[6-(5-cyclopropyl-4H-1,2,4-triazol-3-yl)-2-azaspiro[3.3]heptane-2-carbonyl]-N-[1-(trifluoromethyl)cyclopropyl]-2,6-diazaspiro[3.3]heptane-2-sulfonamide;
6-[6-(5-cyclopropyl-4H-1,2,4-triazol-3-yl)-2-azaspiro[3.3]heptane-2-carbonyl]—N [[1-(trifluoromethyl)cyclopropyl]methyl]-2,6-diazaspiro[3.3]heptane-2-sulfonamide;
1-[2-[6-(5-cyclopropyl-4H-1,2,4-triazol-3-yl)-2-azaspiro[3.3]heptane-2-carbonyl]-2,6-diazaspiro[3.3]heptan-6-yl]-2,2,2-trifluoro-ethanone;
[6-(5-cyclopropyl-4H-1,2,4-triazol-3-yl)-2-azaspiro[3.3]heptan-2-yl]-[6-[5-(trifluoromethyl)pyrazin-2-yl]-2,6-diazaspiro[3.3]heptan-2-yl]methanone;
[6-(5-cyclopropyl-4H-1,2,4-triazol-3-yl)-2-azaspiro[3.3]heptan-2-yl]-[3-[(4,4-difluoro-1-piperidyl)methyl]azetidin-1-yl]methanone;
[6-(5-cyclopropyl-4H-1,2,4-triazol-3-yl)-2-azaspiro[3.3]heptan-2-yl]-[6-(1,1-dioxo-1,4-thiazinan-4-yl)-2-azaspiro[3.3]heptan-2-yl]methanone;
[6-(5-cyclopropyl-4H-1,2,4-triazol-3-yl)-2-azaspiro[3.3]heptan-2-yl]-[6-[[1-(trifluoromethyl)cyclopropyl]methylamino]-2-azaspiro[3.3]heptan-2-yl]methanone;
N-[2-[6-(5-cyclopropyl-4H-1,2,4-triazol-3-yl)-2-azaspiro[3.3]heptane-2-carbonyl]-2-azaspiro[3.3]heptan-6-yl]-1-(trifluoromethyl)cyclopropanecarboxamide;
N-[2-[6-(5-cyclopropyl-4H-1,2,4-triazol-3-yl)-2-azaspiro[3.3]heptane-2-carbonyl]-2-azaspiro[3.3]heptan-6-yl]-4-(trifluoromethyl)benzenesulfonamide;
N-[2-[6-(5-cyclopropyl-4H-1,2,4-triazol-3-yl)-2-azaspiro[3.3]heptane-2-carbonyl]-2-azaspiro[3.3]heptan-6-yl]-2-(trifluoromethyl)benzenesulfonamide;
N-[2-[6-(5-cyclopropyl-4H-1,2,4-triazol-3-yl)-2-azaspiro[3.3]heptane-2-carbonyl]-2-azaspiro[3.3]heptan-6-yl]-2,2-dimethyl-propane-1-sulfonamide;
[6-(5-cyclopropyl-4H-1,2,4-triazol-3-yl)-2-azaspiro[3.3]heptan-2-yl]-[3-[4-[1-(trifluoromethyl)cyclopropyl]phenyl]azetidin-1-yl]methanone;
[6-[5-(1-hydroxycyclopropyl)-4H-1,2,4-triazol-3-yl]-2-azaspiro[3.3]heptan-2-yl]-[3-[4-[1-(trifluoromethyl)cyclopropyl]phenyl]azetidin-1-yl]methanone;
[6-[5-(2,2,2-trifluoro-1-hydroxy-ethyl)-4H-1,2,4-triazol-3-yl]-2-azaspiro[3.3]heptan-2-yl]-[3-[4-[1-(trifluoromethyl)cyclopropyl]phenyl]azetidin-1-yl]methanone;
[6-[5-[(1R)-2,2,2-trifluoro-1-hydroxy-ethyl]-4H-1,2,4-triazol-3-yl]-2-azaspiro[3.3]heptan-2-yl]-[3-[4-[1-(trifluoromethyl)cyclopropyl]phenyl]azetidin-1-yl]methanone;
[6-[5-[(1 S)-2,2,2-trifluoro-1-hydroxy-ethyl]-4H-1,2,4-triazol-3-yl]-2-azaspiro[3.3]heptan-2-yl]-[3-[4-[1-(trifluoromethyl)cyclopropyl]phenyl]azetidin-1-yl]methanone;
[6-(5-cyclopropyl-4H-1,2,4-triazol-3-yl)-2-azaspiro[3.3]heptan-2-yl]-[3-[6-[1-(trifluoromethyl)cyclopropyl]-3-pyridyl]azetidin-1-yl]methanone;
[6-[5-(1-hydroxycyclopropyl)-4H-1,2,4-triazol-3-yl]-2-azaspiro[3.3]heptan-2-yl]-[3-[6-[1-(trifluoromethyl)cyclopropyl]-3-pyridyl]azetidin-1-yl]methanone;
[6-(5-cyclopropyl-4H-1,2,4-triazol-3-yl)-2-azaspiro[3.3]heptan-2-yl]-[3-[5-[1-(trifluoromethyl)cyclopropyl]pyrazin-2-yl]azetidin-1-yl]methanone;
[6-(5-cyclopropyl-4H-1,2,4-triazol-3-yl)-2-azaspiro[3.3]heptan-2-yl]-[3-[6-[1-(trifluoromethyl)cyclopropyl]pyridazin-3-yl]azetidin-1-yl]methanone;
[6-(5-cyclopropyl-4H-1,2,4-triazol-3-yl)-2-azaspiro[3.3]heptan-2-yl]-[3-[2-[1-(trifluoromethyl)cyclopropyl]pyrimidin-5-yl]azetidin-1-yl]methanone;
[6-(5-cyclopropyl-4H-1,2,4-triazol-3-yl)-2-azaspiro[3.3]heptan-2-yl]-[3-[5-[1-(trifluoromethyl)cyclopropyl]-2-pyridyl]azetidin-1-yl]methanone;
[6-(5-cyclopropyl-4H-1,2,4-triazol-3-yl)-2-azaspiro[3.3]heptan-2-yl]-[7-[[3-(trifluoromethyl)-1-bicyclo[1.1.1]pentanyl]sulfonyl]-2,7-diazaspiro[3.5]nonan-2-yl]methanone;
[6-[5-(1-hydroxycyclopropyl)-4H-1,2,4-triazol-3-yl]-2-azaspiro[3.3]heptan-2-yl]-[7-[[3-(trifluoromethyl)-1-bicyclo[1.1.1]pentanyl]sulfonyl]-2,7-diazaspiro[3.5]nonan-2-yl]methanone;
[6-(5-cyclobutyl-4H-1,2,4-triazol-3-yl)-2-azaspiro[3.3]heptan-2-yl]-[7-[[3-(trifluoromethyl)-1-bicyclo[1.1.1]pentanyl]sulfonyl]-2,7-diazaspiro[3.5]nonan-2-yl]methanone;
[6-(5-cyclopropyl-4H-1,2,4-triazol-3-yl)-2-azaspiro[3.3]heptan-2-yl]-[7-[2-fluoro-4-(trifluoromethyl)phenyl]sulfonyl-2,7-diazaspiro[3.5]nonan-2-yl]methanone;
[6-(5-cyclopropyl-4H-1,2,4-triazol-3-yl)-2-azaspiro[3.3]heptan-2-yl]-[3-[3-fluoro-4-(trifluoromethoxy)phenyl]azetidin-1-yl]methanone;
[3-[3-fluoro-4-(trifluoromethoxy)phenyl]azetidin-1-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[3-[4-(trifluoromethylsulfonyl)phenyl]azetidin-1-yl]methanone;
[6-[5-(1-hydroxycyclopropyl)-4H-1,2,4-triazol-3-yl]-2-azaspiro[3.3]heptan-2-yl]-[3-[4-(2,2,2-trifluoroethyl)phenyl]azetidin-1-yl]methanone;
[6-(5-cyclopropyl-4H-1,2,4-triazol-3-yl)-2-azaspiro[3.3]heptan-2-yl]-[3-[3-[[1-(trifluoromethyl)cyclopropyl]methyl]-1,2,4-oxadiazol-5-yl]azetidin-1-yl]methanone;
[6-(5-cyclopropyl-4H-1,2,4-triazol-3-yl)-2-azaspiro[3.3]heptan-2-yl]-[3-[5-[1-(trifluoromethyl)cyclopropyl]-1,2,4-oxadiazol-3-yl]azetidin-1-yl]methanone;
[3-(2-chloro-4-isopropylsulfonyl-phenyl)azetidin-1-yl]-[6-(5-cyclopropyl-4H-1,2,4-triazol-3-yl)-2-azaspiro[3.3]heptan-2-yl]methanone;
N-[1-[6-(5-cyclopropyl-4H-1,2,4-triazol-3-yl)-2-azaspiro[3.3]heptane-2-carbonyl]-4-piperidyl]-4-(trifluoromethyl)benzenesulfonamide;
N-[1-[6-(5-cyclopropyl-4H-1,2,4-triazol-3-yl)-2-azaspiro[3.3]heptane-2-carbonyl]-4-piperidyl]-4-fluoro-benzenesulfonamide;
N-[[1-[6-(5-cyclopropyl-4H-1,2,4-triazol-3-yl)-2-azaspiro[3.3]heptane-2-carbonyl]-4-piperidyl]methyl]-4-(trifluoromethyl)benzenesulfonamide;
N-[[1-[6-(5-cyclopropyl-4H-1,2,4-triazol-3-yl)-2-azaspiro[3.3]heptane-2-carbonyl]-4-piperidyl]methyl]-4-(trifluoromethoxy)benzenesulfonamide;
4-chloro-N-[[1-[6-(5-cyclopropyl-4H-1,2,4-triazol-3-yl)-2-azaspiro[3.3]heptane-2-carbonyl]-4-piperidyl]methyl]benzenesulfonamide;
[6-(5-cyclopropyl-4H-1,2,4-triazol-3-yl)-2-azaspiro[3.3]heptan-2-yl]-[3-[6-[[1-(trifluoromethyl)cyclopropyl]methylamino]-3-pyridyl]azetidin-1-yl]methanone;
[6-[5-(1-hydroxycyclopropyl)-4H-1,2,4-triazol-3-yl]-2-azaspiro[3.3]heptan-2-yl]-[3-[6-[[1-(trifluoromethyl)cyclopropyl]methylamino]-3-pyridyl]azetidin-1-yl]methanone;
[6-(5-cyclopropyl-4H-1,2,4-triazol-3-yl)-2-azaspiro[3.3]heptan-2-yl]-[3-[5-[[1-(trifluoromethyl)cyclopropyl]methylamino]pyrazin-2-yl]azetidin-1-yl]methanone;
[6-(5-cyclopropyl-4H-1,2,4-triazol-3-yl)-2-azaspiro[3.3]heptan-2-yl]-[3-[5-[[1-(trifluoromethyl)cyclopropyl]methylamino]-2-pyridyl]azetidin-1-yl]methanone;
[6-(5-cyclopropyl-4H-1,2,4-triazol-3-yl)-2-azaspiro[3.3]heptan-2-yl]-[3-[4-[[1-(trifluoromethyl)cyclopropyl]methylamino]phenyl]azetidin-1-yl]methanone;
[6-(5-cyclopropyl-4H-1,2,4-triazol-3-yl)-2-azaspiro[3.3]heptan-2-yl]-[3-[6-[3-(trifluoromethyl)azetidin-1-yl]-3-pyridyl]azetidin-1-yl]methanone;
[6-[5-(1-hydroxycyclopropyl)-4H-1,2,4-triazol-3-yl]-2-azaspiro[3.3]heptan-2-yl]-[3-[6-[3-(trifluoromethyl)azetidin-1-yl]-3-pyridyl]azetidin-1-yl]methanone;
[6-(5-cyclobutyl-4H-1,2,4-triazol-3-yl)-2-azaspiro[3.3]heptan-2-yl]-[3-[6-[3-(trifluoromethyl)azetidin-1-yl]-3-pyridyl]azetidin-1-yl]methanone;
[6-(5-cyclopropyl-4H-1,2,4-triazol-3-yl)-2-azaspiro[3.3]heptan-2-yl]-[3-[4-[3-(trifluoromethyl)azetidin-1-yl]phenyl]azetidin-1-yl]methanone;
[6-[5-(1-hydroxycyclopropyl)-4H-1,2,4-triazol-3-yl]-2-azaspiro[3.3]heptan-2-yl]-[3-[4-[3-(trifluoromethyl)azetidin-1-yl]phenyl]azetidin-1-yl]methanone;
[6-(5-cyclopropyl-4H-1,2,4-triazol-3-yl)-2-azaspiro[3.3]heptan-2-yl]-[3-[2-[3-(trifluoromethyl)azetidin-1-yl]pyrimidin-5-yl]azetidin-1-yl]methanone;
[6-(5-cyclopropyl-4H-1,2,4-triazol-3-yl)-2-azaspiro[3.3]heptan-2-yl]-[3-[2-[3-(trifluoromethyl)pyrrolidin-1-yl]pyrimidin-5-yl]azetidin-1-yl]methanone;
[6-(5-cyclopropyl-4H-1,2,4-triazol-3-yl)-2-azaspiro[3.3]heptan-2-yl]-[3-[2-[(3R)-3-(trifluoromethyl)pyrrolidin-1-yl]pyrimidin-5-yl]azetidin-1-yl]methanone;
[6-(5-cyclopropyl-4H-1,2,4-triazol-3-yl)-2-azaspiro[3.3]heptan-2-yl]-[3-[2-[(3 S)-3-(trifluoromethyl)pyrrolidin-1-yl]pyrimidin-5-yl]azetidin-1-yl]methanone;
[6-(5-cyclopropyl-4H-1,2,4-triazol-3-yl)-2-azaspiro[3.3]heptan-2-yl]-[3-[6-[(3 S)-3-(trifluoromethyl)pyrrolidin-1-yl]-3-pyridyl]azetidin-1-yl]methanone;
[6-(5-cyclopropyl-4H-1,2,4-triazol-3-yl)-2-azaspiro[3.3]heptan-2-yl]-[3-[6-[(3R)-3-(trifluoromethyl)pyrrolidin-1-yl]-3-pyridyl]azetidin-1-yl]methanone;
[6-(5-cyclopropyl-4H-1,2,4-triazol-3-yl)-2-azaspiro[3.3]heptan-2-yl]-[3-[6-[3-hydroxy-3-(trifluoromethyl)pyrrolidin-1-yl]-3-pyridyl]azetidin-1-yl]methanone;
[6-(5-cyclopropyl-4H-1,2,4-triazol-3-yl)-2-azaspiro[3.3]heptan-2-yl]-[3-[6-[rac-(3R)-3-hydroxy-3-(trifluoromethyl)pyrrolidin-1-yl]-3-pyridyl]azetidin-1-yl]methanone;
[6-(5-cyclopropyl-4H-1,2,4-triazol-3-yl)-2-azaspiro[3.3]heptan-2-yl]-[3-[6-[rac-(3 S)-3-hydroxy-3-(trifluoromethyl)pyrrolidin-1-yl]-3-pyridyl]azetidin-1-yl]methanone;
[6-[5-(1-hydroxycyclopropyl)-4H-1,2,4-triazol-3-yl]-2-azaspiro[3.3]heptan-2-yl]-[3-[6-[[1-(trifluoromethyl)cyclopropyl]amino]-3-pyridyl]azetidin-1-yl]methanone;
[6-(5-cyclobutyl-4H-1,2,4-triazol-3-yl)-2-azaspiro[3.3]heptan-2-yl]-[3-[6-[[1-(trifluoromethyl)cyclopropyl]amino]-3-pyridyl]azetidin-1-yl]methanone;
[6-(5-cyclopropyl-4H-1,2,4-triazol-3-yl)-2-azaspiro[3.3]heptan-2-yl]-[3-[4-[[1-(trifluoromethyl)cyclopropyl]amino]phenyl]azetidin-1-yl]methanone;
[6-(5-cyclopropyl-4H-1,2,4-triazol-3-yl)-2-azaspiro[3.3]heptan-2-yl]-[3-[6-[3-hydroxy-3-(trifluoromethyl)azetidin-1-yl]-3-pyridyl]azetidin-1-yl]methanone;
[6-(5-Cyclopropyl-4H-1,2,4-triazol-3-yl)-2-azaspiro[3.3]heptan-2-yl]-[3-[4-(3,5-diisopropylpyrazol-1-yl)phenyl]azetidin-1-yl]methanone;
[3-[4-(3-tert-Butyl-5-methyl-pyrazol-1-yl)phenyl]azetidin-1-yl]-[6-(5-cyclopropyl-4H-1,2,4-triazol-3-yl)-2-azaspiro[3.3]heptan-2-yl]methanone;
[6-(5-Cyclopropyl-4H-1,2,4-triazol-3-yl)-2-azaspiro[3.3]heptan-2-yl]-[3-[4-[5-methyl-3-(trifluoromethyl)pyrazol-1-yl]phenyl]azetidin-1-yl]methanone;
(4R) or (4S)-1-[4-[1-[6-(5-Cyclopropyl-4H-1,2,4-triazol-3-yl)-2-azaspiro[3.3]heptane-2-carbonyl]azetidin-3-yl]phenyl]-4-(trifluoromethyl)piperidin-2-one;
(4S) or (4R)-1-[4-[1-[6-(5-Cyclopropyl-4H-1,2,4-triazol-3-yl)-2-azaspiro[3.3]heptane-2-carbonyl]azetidin-3-yl]phenyl]-4-(trifluoromethyl)piperidin-2-one;
[7-(5-Chloro-3-fluoro-2-pyridyl)-2,7-diazaspiro[3.5]nonan-2-yl]-[6-(5-cyclopropyl-4H-1,2,4-triazol-3-yl)-2-azaspiro[3.3]heptan-2-yl]methanone;
[3-[4-[3-(Difluoromethyl)cyclobutyl]phenyl]azetidin-1-yl]-[6-[5-(1-hydroxycyclopropyl)-4H-1,2,4-triazol-3-yl]-2-azaspiro[3.3]heptan-2-yl]methanone;
[7-(4-Chloro-2-fluoro-phenyl)-2,7-diazaspiro[3.5]nonan-2-yl]-[6-(5-cyclopropyl-4H-1,2,4-triazol-3-yl)-2-azaspiro[3.3]heptan-2-yl]methanone;
1-[4-[1-[6-(5-Cyclopropyl-4H-1,2,4-triazol-3-yl)-2-azaspiro[3.3]heptane-2-carbonyl]azetidin-3-yl]phenyl]-4-(trifluoromethyl)pyridin-2-one;
[6-(5-Cyclopropyl-4H-1,2,4-triazol-3-yl)-2-azaspiro[3.3]heptan-2-yl]-[3-[4-[3-(difluoromethyl)-5-methyl-pyrazol-1-yl]phenyl]azetidin-1-yl]methanone;
[6-(5-Cyclopropyl-4H-1,2,4-triazol-3-yl)-2-azaspiro[3.3]heptan-2-yl]-[3-(5-spiro[3.3]heptan-2-ylpyrazin-2-yl)azetidin-1-yl]methanone;
[3-[4-(5-Cyclopropyl-3-methyl-pyrazol-1-yl)phenyl]azetidin-1-yl]-[6-(5-cyclopropyl-4H-1,2,4-triazol-3-yl)-2-azaspiro[3.3]heptan-2-yl]methanone;
[3-[4-(3-Cyclopropyl-5-methyl-pyrazol-1-yl)phenyl]azetidin-1-yl]-[6-(5-cyclopropyl-4H-1,2,4-triazol-3-yl)-2-azaspiro[3.3]heptan-2-yl]methanone;
[6-(5-Cyclopropyl-4H-1,2,4-triazol-3-yl)-2-azaspiro[3.3]heptan-2-yl]-[3-[6-(2,2-difluoro-5-azaspiro[2.4]heptan-5-yl)-3-pyridyl]azetidin-1-yl]methanone;
[6-(5-Cyclopropyl-4H-1,2,4-triazol-3-yl)-2-azaspiro[3.3]heptan-2-yl]-[3-[6-(6-oxa-2-azaspiro[3.4]octan-2-yl)-3-pyridyl]azetidin-1-yl]methanone;
[6-(5-Cyclopropyl-4H-1,2,4-triazol-3-yl)-2-azaspiro[3.3]heptan-2-yl]-[3-[4-[3-hydroxy-3-(trifluoromethyl)cyclobutyl]phenyl]azetidin-1-yl]methanone;
[6-(5-Cyclopropyl-4H-1,2,4-triazol-3-yl)-2-azaspiro[3.3]heptan-2-yl]-[3-[5-[3-(trifluoromethyl)azetidin-1-yl]-2-pyridyl]azetidin-1-yl]methanone;
[6-(5-Cyclopropyl-4H-1,2,4-triazol-3-yl)-2-azaspiro[3.3]heptan-2-yl]-[3-[6-(5-oxa-2-azaspiro[3.4]octan-2-yl)-3-pyridyl]azetidin-1-yl]methanone;
[6-(5-Cyclopropyl-4H-1,2,4-triazol-3-yl)-2-azaspiro[3.3]heptan-2-yl]-[3-[4-(3,5-dimethylpyrazol-1-yl)phenyl]azetidin-1-yl]methanone;
[6-(5-Cyclopropyl-4H-1,2,4-triazol-3-yl)-2-azaspiro[3.3]heptan-2-yl]-[3-[4-[1-(hydroxymethyl)cyclopropyl]phenyl]azetidin-1-yl]methanone;
[6-(5-Cyclopropyl-4H-1,2,4-triazol-3-yl)-2-azaspiro[3.3]heptan-2-yl]-[3-[6-(2-oxaspiro[3.3]heptan-6-yl)-3-pyridyl]azetidin-1-yl]methanone; 4-methylbenzenesulfonic acid;
[3-(4-Cyclobutyl-3-methylsulfonyl-phenyl)azetidin-1-yl]-[6-(5-cyclopropyl-4H-1,2,4-triazol-3-yl)-2-azaspiro[3.3]heptan-2-yl]methanone;
[6-(5-Cyclopropyl-4H-1,2,4-triazol-3-yl)-2-azaspiro[3.3]heptan-2-yl]-[3-[6-[3-(1-hydroxy-1-methyl-ethyl)azetidin-1-yl]-3-pyridyl]azetidin-1-yl]methanone;
[6-(5-Cyclopropyl-4H-1,2,4-triazol-3-yl)-2-azaspiro[3.3]heptan-2-yl]-[3-[6-(2,2-dimethylpropyl)-3-pyridyl]azetidin-1-yl]methanone;
(trans)-[6-(5-Cyclopropyl-4H-1,2,4-triazol-3-yl)-2-azaspiro[3.3]heptan-2-yl]-[3-[6-[3-(trifluoromethyl)cyclobutyl]-3-pyridyl]azetidin-1-yl]methanone;
(trans)-[6-(5-Cyclopropyl-4H-1,2,4-triazol-3-yl)-2-azaspiro[3.3]heptan-2-yl]-[3-[6-[3-(difluoromethyl)cyclobutyl]-3-pyridyl]azetidin-1-yl]methanone;
[3-[6-(2-Azaspiro[3.4]octan-2-yl)-3-pyridyl]azetidin-1-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[3-[6-(2-oxa-7-azaspiro[3.4]octan-7-yl)-3-pyridyl]azetidin-1-yl]methanone;
trans-[6-(5-Cyclopropyl-4H-1,2,4-triazol-3-yl)-2-azaspiro[3.3]heptan-2-yl]-[3-[4-[3-(difluoromethyl)cyclobutyl]phenyl]azetidin-1-yl]methanone;
cis-[6-(5-Cyclopropyl-4H-1,2,4-triazol-3-yl)-2-azaspiro[3.3]heptan-2-yl]-[3-[4-[3-(difluoromethyl)cyclobutyl]phenyl]azetidin-1-yl]methanone;
[6-(5-Cyclopropyl-4H-1,2,4-triazol-3-yl)-2-azaspiro[3.3]heptan-2-yl]-[3-hydroxy-3-[4-[3-(trifluoromethyl)azetidin-1-yl]phenyl]azetidin-1-yl]methanone;
[6-(5-cyclopropyl-4H-1,2,4-triazol-3-yl)-2-azaspiro[3.3]heptan-2-yl]-[3-[[2-methoxy-4-(trifluoromethyl)phenyl]methoxy]azetidin-1-yl]methanone;
[6-(5-cyclopropyl-4H-1,2,4-triazol-3-yl)-2-azaspiro[3.3]heptan-2-yl]-[3-[[2-methoxy-4-(trifluoromethyl)phenyl]methoxy]azetidin-1-yl]methanone;
[6-(5-cyclopropyl-4H-1,2,4-triazol-3-yl)-2-azaspiro[3.3]heptan-2-yl]-[3-[[2-fluoro-4-(trifluoromethylsulfonyl)phenyl]methoxy]azetidin-1-yl]methanone;
2-cyclopropyl-6-[1-[6-(5-cyclopropyl-4H-1,2,4-triazol-3-yl)-2-azaspiro[3.3]heptane-2-carbonyl]azetidin-3-yl]oxy-benzonitrile;
[6-(5-cyclopropyl-4H-1,2,4-triazol-3-yl)-2-azaspiro[3.3]heptan-2-yl]-[3-[[2-fluoro-4-(trifluoromethyl)phenyl]methoxy]azetidin-1-yl]methanone;
[3-(4-cyclopropylphenoxy)azetidin-1-yl]-[6-[5-(1-hydroxycyclopropyl)-4H-1,2,4-triazol-3-yl]-2-azaspiro[3.3]heptan-2-yl]methanone;
[3-(4-cyclopropyl-2-fluoro-phenoxy)azetidin-1-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[3-[6-(trifluoromethyl)pyridazin-3-yl]oxyazetidin-1-yl]methanone;
[6-(5-cyclopropyl-4H-1,2,4-triazol-3-yl)-2-azaspiro[3.3]heptan-2-yl]-[3-[[4-(trifluoromethoxy)phenyl]methoxy]azetidin-1-yl]methanone;
[6-(5-cyclopropyl-4H-1,2,4-triazol-3-yl)-2-azaspiro[3.3]heptan-2-yl]-[3-[[4-(trifluoromethylsulfonyl)phenyl]methoxy]azetidin-1-yl]methanone;
5-cyclopropyl-2-[1-[6-[5-(1-hydroxycyclopropyl)-4H-1,2,4-triazol-3-yl]-2-azaspiro[3.3]heptane-2-carbonyl]azetidin-3-yl]oxy-benzonitrile;
methyl 2-[3-[1-[6-(5-cyclopropyl-4H-1,2,4-triazol-3-yl)-2-azaspiro[3.3]heptane-2-carbonyl]azetidin-3-yl]oxyphenyl]-2-methyl-propanoate;
[3-[3-cyclopropyl-4-(trifluoromethyl)phenoxy]azetidin-1-yl]-[6-[5-(1-hydroxycyclopropyl)-4H-1,2,4-triazol-3-yl]-2-azaspiro[3.3]heptan-2-yl]methanone;
[3-[(2-chloro-4-fluoro-phenyl)methoxy]azetidin-1-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[3-[[3-(trifluoromethylsulfonyl)phenyl]methoxy]azetidin-1-yl]methanone;
[3-(3-cyclopropyl-2-fluoro-phenoxy)azetidin-1-yl]-[6-[5-(1-hydroxycyclopropyl)-4H-1,2,4-triazol-3-yl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[3-[4-(trifluoromethyl)phenoxy]azetidin-1-yl]methanone;
[3-[[2-fluoro-4-(trifluoromethyl)phenyl]methoxy]azetidin-1-yl]-[6-[5-(1-hydroxycyclopropyl)-4H-1,2,4-triazol-3-yl]-2-azaspiro[3.3]heptan-2-yl]methanone;
[3-(4-chloro-3-cyclopropyl-phenoxy)azetidin-1-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[3-[(4,4-difluorocyclohexyl)methoxy]azetidin-1-yl]methanone;
[6-(5-cyclopropyl-4H-1,2,4-triazol-3-yl)-2-azaspiro[3.3]heptan-2-yl]-[3-[(6,6-difluoro-3-bicyclo[3.1.0]hexanyl)methoxy]azetidin-1-yl]methanone;
[3-[(6-chloro-5-cyclopropyl-3-pyridyl)oxy]azetidin-1-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[3-[[5-(trifluoromethyl)-3-pyridyl]methoxy]azetidin-1-yl]methanone;
[6-(5-cyclopropyl-4H-1,2,4-triazol-3-yl)-2-azaspiro[3.3]heptan-2-yl]-[3-[(2-fluoro-4-methylsulfonyl-phenyl)methoxy]azetidin-1-yl]methanone;
[6-(5-cyclopropyl-4H-1,2,4-triazol-3-yl)-2-azaspiro[3.3]heptan-2-yl]-[3-[[1-(trifluoromethyl)cyclopropyl]methoxy]azetidin-1-yl]methanone;
[6-(5-cyclopropyl-4H-1,2,4-triazol-3-yl)-2-azaspiro[3.3]heptan-2-yl]-[3-[[6-(trifluoromethyl)-3-pyridyl]methoxy]azetidin-1-yl]methanone;
[6-(5-cyclopropyl-4H-1,2,4-triazol-3-yl)-2-azaspiro[3.3]heptan-2-yl]-[3-[(4-methylsulfonylphenyl)methoxy]azetidin-1-yl]methanone;
[6-(5-cyclopropyl-4H-1,2,4-triazol-3-yl)-2-azaspiro[3.3]heptan-2-yl]-[3-[(3-methylsulfonylphenyl)methoxy]azetidin-1-yl]methanone;
[6-(5-cyclopropyl-4H-1,2,4-triazol-3-yl)-2-azaspiro[3.3]heptan-2-yl]-[3-[(2-methylsulfonylphenyl)methoxy]azetidin-1-yl]methanone;
[3-[[2-fluoro-4-(trifluoromethyl)phenyl]methoxy]azetidin-1-yl]-[6-[5-(2,2,2-trifluoro-1-hydroxy-ethyl)-4H-1,2,4-triazol-3-yl]-2-azaspiro[3.3]heptan-2-yl]methanone;
[3-[4-(4-chloro-2-methylsulfonyl-phenyl)phenyl]azetidin-1-yl]-[6-(5-cyclopropyl-4H-1,2,4-triazol-3-yl)-2-azaspiro[3.3]heptan-2-yl]methanone;
[3-[4-(2-chloro-4-methylsulfonyl-phenyl)phenyl]azetidin-1-yl]-[6-(5-cyclopropyl-4H-1,2,4-triazol-3-yl)-2-azaspiro[3.3]heptan-2-yl]methanone;
methyl 2-[4-[1-[6-(5-cyclopropyl-4H-1,2,4-triazol-3-yl)-2-azaspiro[3.3]heptane-2-carbonyl]azetidin-3-yl]phenyl]benzoate;
2-[4-[1-[6-(5-cyclopropyl-4H-1,2,4-triazol-3-yl)-2-azaspiro[3.3]heptane-2-carbonyl]azetidin-3-yl]phenyl]benzamide;
[6-(5-cyclopropyl-4H-1,2,4-triazol-3-yl)-2-azaspiro[3.3]heptan-2-yl]-[3-[3-[[[1-(trifluoromethyl)cyclopropyl]amino]methyl]-1-bicyclo[1.1.1]pentanyl]azetidin-1-yl]methanone;
[6-(5-cyclopropyl-4H-1,2,4-triazol-3-yl)-2-azaspiro[3.3]heptan-2-yl]-[3-[3-[[1-(trifluoromethyl)cyclopropyl]methylamino]-1-bicyclo[1.1.1]pentanyl]azetidin-1-yl]methanone;
[6-(5-cyclopropyl-4H-1,2,4-triazol-3-yl)-2-azaspiro[3.3]heptan-2-yl]-[3-[3-[5-(2,2-dimethylpropyl)-1,3,4-oxadiazol-2-yl]-1-bicyclo[1.1.1]pentanyl]azetidin-1-yl]methanone;
[6-(5-cyclopropyl-4H-1,2,4-triazol-3-yl)-2-azaspiro[3.3]heptan-2-yl]-[3-[3-[5-(2,2,2-trifluoroethyl)-1,3,4-oxadiazol-2-yl]-1-bicyclo[1.1.1]pentanyl]azetidin-1-yl]methanone;
[3-[3-(5-cyclopropyl-1,3,4-oxadiazol-2-yl)-1-bicyclo[1.1.1]pentanyl]azetidin-1-yl]-[6-(5-cyclopropyl-4H-1,2,4-triazol-3-yl)-2-azaspiro[3.3]heptan-2-yl]methanone;
[7-(2,5-dichlorophenoxy)-2-azaspiro[3.5]nonan-2-yl]-[6-[5-(1-hydroxycyclopropyl)-4H-1,2,4-triazol-3-yl]-2-azaspiro[3.3]heptan-2-yl]methanone;
[6-(5-cyclopropyl-4H-1,2,4-triazol-3-yl)-2-azaspiro[3.3]heptan-2-yl]-[3-[[2-fluoro-4-(trifluoromethyl)phenoxy]methyl]azetidin-1-yl]methanone;
N-[2-[6-(5-cyclopropyl-4H-1,2,4-triazol-3-yl)-2-azaspiro[3.3]heptane-2-carbonyl]-2-azaspiro[3.5]nonan-7-yl]-3-(trifluoromethoxy)benzenesulfonamide;
[3-[2-[2-fluoro-4-(trifluoromethyl)phenyl]ethyl]azetidin-1-yl]-[6-[3-(1-hydroxycyclopropyl)-1H-1,2,4-triazol-5-yl]-2-azaspiro[3.3]heptan-2-yl]methanone;
[7-(5-chloro-2-methylsulfonyl-phenoxy)-2-azaspiro[3.5]nonan-2-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[7-[[5-(trifluoromethyl)pyrazin-2-yl]amino]-2-azaspiro[3.5]nonan-2-yl]methanone;
[6-(5-cyclopropyl-4H-1,2,4-triazol-3-yl)-2-azaspiro[3.3]heptan-2-yl]-[6-[[[1-(trifluoromethyl)cyclopropyl]methylamino]methyl]-2-azaspiro[3.3]heptan-2-yl]methanone;
[3-[(2-chloro-4-fluoro-phenoxy)methyl]azetidin-1-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[6-[[[3-(trifluoromethyl)oxetan-3-yl]amino]methyl]-2-azaspiro[3.3]heptan-2-yl]methanone;
[6-(5-cyclopropyl-4H-1,2,4-triazol-3-yl)-2-azaspiro[3.3]heptan-2-yl]-[7-(4-fluoro-2-methylsulfonyl-phenoxy)-2-azaspiro[3.5]nonan-2-yl]methanone;
[6-(2-chloro-4-fluoro-phenyl)sulfonyl-2-azaspiro[3.3]heptan-2-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[6-[[(3-methyl-1,1-dioxo-thietan-3-yl)amino]methyl]-2-azaspiro[3.3]heptan-2-yl]methanone;
N-[2-[6-(5-cyclopropyl-4H-1,2,4-triazol-3-yl)-2-azaspiro[3.3]heptane-2-carbonyl]-2-azaspiro[3.5]nonan-7-yl]-4,4-difluoro-cyclohexanesulfonamide;
2-[6-(5-cyclopropyl-4H-1,2,4-triazol-3-yl)-2-azaspiro[3.3]heptane-2-carbonyl]—N-[[1-(trifluoromethyl)cyclopropyl]methyl]-2-azaspiro[3.3]heptane-6-carboxamide;
[6-(5-cyclopropyl-4H-1,2,4-triazol-3-yl)-2-azaspiro[3.3]heptan-2-yl]-[3-[1-[[4-(trifluoromethyl)phenyl]methyl]azetidin-3-yl]azetidin-1-yl]methanone;
[3-[(4-chlorophenyl)methyl]azetidin-1-yl]-[6-[5-(1-hydroxycyclopropyl)-4H-1,2,4-triazol-3-yl]-2-azaspiro[3.3]heptan-2-yl]methanone;
2-[1-[6-(5-cyclopropyl-4H-1,2,4-triazol-3-yl)-2-azaspiro[3.3]heptane-2-carbonyl]-4-piperidyl]-2-(4-fluorophenyl)acetamide;
[(3aR,6aS)-2-benzyl-1,3,3a,4,6,6a-hexahydropyrrolo[3,4-c]pyrrol-5-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[6-[[(1,1-dioxothietan-3-yl)amino]methyl]-2-azaspiro[3.3]heptan-2-yl]methanone;
[3-[4-[1-(2-tert-butyltetrazol-5-yl)cyclopropyl]phenyl]azetidin-1-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[3-[4-(2,2,2-trifluoro-1,1-dimethyl-ethoxy)phenyl]azetidin-1-yl]methanone;
[3-[6-(2-chlorophenoxy)-3-pyridyl]azetidin-1-yl]-[6-(5-cyclopropyl-4H-1,2,4-triazol-3-yl)-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[3-[4-(2,2,2-trifluoro-1,1-dimethyl-ethoxy)phenyl]azetidin-1-yl]methanone;
[6-(5-cyclopropyl-4H-1,2,4-triazol-3-yl)-2-azaspiro[3.3]heptan-2-yl]-[3-[4-[3-(2,2-dimethylpropyl)triazol-4-yl]phenyl]azetidin-1-yl]methanone;
[6-(5-cyclopropyl-4H-1,2,4-triazol-3-yl)-2-azaspiro[3.3]heptan-2-yl]-[3-[4-[5-(trifluoromethyl)pyrazin-2-yl]oxyphenyl]azetidin-1-yl]methanone;
[3-[4-(benzenesulfonyl)phenyl]azetidin-1-yl]-[6-(5-cyclopropyl-4H-1,2,4-triazol-3-yl)-2-azaspiro[3.3]heptan-2-yl]methanone;
methyl 1-[4-[1-[6-(5-cyclopropyl-4H-1,2,4-triazol-3-yl)-2-azaspiro[3.3]heptane-2-carbonyl]azetidin-3-yl]phenyl]cyclopropanecarboxylate;
[3-(4-cyclohexylsulfonylphenyl)azetidin-1-yl]-[6-(5-cyclopropyl-4H-1,2,4-triazol-3-yl)-2-azaspiro[3.3]heptan-2-yl]methanone;
[3-[6-(2-chlorophenoxy)-3-pyridyl]azetidin-1-yl]-[6-[5-(1-hydroxycyclopropyl)-4H-1,2,4-triazol-3-yl]-2-azaspiro[3.3]heptan-2-yl]methanone;
[6-[5-(1-hydroxycyclopropyl)-4H-1,2,4-triazol-3-yl]-2-azaspiro[3.3]heptan-2-yl]-[3-[4-[[1-(trifluoromethyl)cyclopropyl]methoxy]phenyl]azetidin-1-yl]methanone;
1-[4-[1-[6-(5-cyclopropyl-4H-1,2,4-triazol-3-yl)-2-azaspiro[3.3]heptane-2-carbonyl]azetidin-3-yl]phenyl]cyclopropanecarbonitrile;
[6-(3-cyclopropyl-1H-1,2,4-triazol-5-yl)-2-azaspiro[3.3]heptan-2-yl]-[3-[4-[5-[1-(trifluoromethyl)cyclopropyl]-4H-1,2,4-triazol-3-yl]phenyl]azetidin-1-yl]methanone;
[3-[6-(2-chlorophenoxy)pyridazin-3-yl]azetidin-1-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[3-[4-[1-(1H-tetrazol-5-yl)cyclopropyl]phenyl]azetidin-1-yl]methanone;
4-[1-[6-(5-cyclopropyl-4H-1,2,4-triazol-3-yl)-2-azaspiro[3.3]heptane-2-carbonyl]azetidin-3-yl]benzenesulfonamide;
[6-(5-cyclopropyl-4H-1,2,4-triazol-3-yl)-2-azaspiro[3.3]heptan-2-yl]-[3-[4-(dimethylphosphorylmethyl)phenyl]azetidin-1-yl]methanone;
[6-(5-cyclopropyl-4H-1,2,4-triazol-3-yl)-2-azaspiro[3.3]heptan-2-yl]-[3-[6-(4-isopropyl-N-methyl-anilino)-3-pyridyl]azetidin-1-yl]methanone;
[6-(5-cyclopropyl-4H-1,2,4-triazol-3-yl)-2-azaspiro[3.3]heptan-2-yl]-[3-[6-(N-methylanilino)-3-pyridyl]azetidin-1-yl]methanone;
1-[4-[1-[6-(5-cyclopropyl-4H-1,2,4-triazol-3-yl)-2-azaspiro[3.3]heptane-2-carbonyl]azetidin-3-yl]phenyl]piperidine-2-carboxamide;
1-[4-[1-[6-(5-cyclopropyl-4H-1,2,4-triazol-3-yl)-2-azaspiro[3.3]heptane-2-carbonyl]azetidin-3-yl]phenyl]-4,4-difluoro-piperidine-2-carboxamide;
1-[4-[1-[6-(5-cyclopropyl-4H-1,2,4-triazol-3-yl)-2-azaspiro[3.3]heptane-2-carbonyl]azetidin-3-yl]phenyl]pyrrolidine-2-carboxamide;
2-[3-[1-[6-(5-cyclopropyl-4H-1,2,4-triazol-3-yl)-2-azaspiro[3.3]heptane-2-carbonyl]azetidin-3-yl]oxyphenyl]-2-methyl-propanoic acid;
[3-[[2-fluoro-4-(trifluoromethyl)phenyl]methoxy]azetidin-1-yl]-[6-[5-[(1R)-2,2,2-trifluoro-1-hydroxy-ethyl]-4H-1,2,4-triazol-3-yl]-2-azaspiro[3.3]heptan-2-yl]methanone;
[3-[[2-fluoro-4-(trifluoromethyl)phenyl]methoxy]azetidin-1-yl]-[6-[5-[(1 S)-2,2,2-trifluoro-1-hydroxy-ethyl]-4H-1,2,4-triazol-3-yl]-2-azaspiro[3.3]heptan-2-yl]methanone;
2-[4-[1-[6-(5-cyclopropyl-4H-1,2,4-triazol-3-yl)-2-azaspiro[3.3]heptane-2-carbonyl]azetidin-3-yl]phenyl]benzoic acid;
2-[4-[1-[6-(5-cyclopropyl-4H-1,2,4-triazol-3-yl)-2-azaspiro[3.3]heptane-2-carbonyl]azetidin-3-yl]phenyl]-N-ethyl-benzamide;
1-[4-[1-[6-(5-cyclopropyl-4H-1,2,4-triazol-3-yl)-2-azaspiro[3.3]heptane-2-carbonyl]azetidin-3-yl]phenyl]cyclopropanecarboxylic acid;
1-[4-[1-[6-(5-cyclopropyl-4H-1,2,4-triazol-3-yl)-2-azaspiro[3.3]heptane-2-carbonyl]azetidin-3-yl]phenyl]cyclopropanecarboxamide;
(2S)-1-[4-[1-[6-(5-cyclopropyl-4H-1,2,4-triazol-3-yl)-2-azaspiro[3.3]heptane-2-carbonyl]azetidin-3-yl]phenyl]piperidine-2-carboxamide;
(2S)-1-[4-[1-[6-(5-cyclopropyl-4H-1,2,4-triazol-3-yl)-2-azaspiro[3.3]heptane-2-carbonyl]azetidin-3-yl]phenyl]-4,4-difluoro-piperidine-2-carboxamide;
(2R)-1-[4-[1-[6-(5-cyclopropyl-4H-1,2,4-triazol-3-yl)-2-azaspiro[3.3]heptane-2-carbonyl]azetidin-3-yl]phenyl]-4,4-difluoro-piperidine-2-carboxamide;
(2S)-1-[4-[1-[6-(5-cyclopropyl-4H-1,2,4-triazol-3-yl)-2-azaspiro[3.3]heptane-2-carbonyl]azetidin-3-yl]phenyl]pyrrolidine-2-carboxamide;
(2R)-1-[4-[1-[6-(5-cyclopropyl-4H-1,2,4-triazol-3-yl)-2-azaspiro[3.3]heptane-2-carbonyl]azetidin-3-yl]phenyl]pyrrolidine-2-carboxamide;
[3-(6-tert-butyl-3-pyridyl)azetidin-1-yl]-[6-(3-cyclopropyl-1H-1,2,4-triazol-5-yl)-2-azaspiro[3.3]heptan-2-yl]methanone;
[[6-(5-cyclopropyl-4H-1,2,4-triazol-3-yl)-2-azaspiro[3.3]heptan-2-yl]-[6-[[6-(trifluoromethyl)pyrimidin-4-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-(trifluoromethyl)pyrazol-4-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[2-(trifluoromethyl)pyrimidin-4-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[4-(trifluoromethyl)pyrimidin-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)-1H-pyrazol-5-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-cyclopropyl-3-(trifluoromethyl)pyrazol-4-yl]methyl]-2-azaspiro[3.3]heptan-2-yl]methanone;
1-[[2-[6-(3-cyclopropyl-1H-1,2,4-triazol-5-yl)-2-azaspiro[3.3]heptane-2-carbonyl]-2-azaspiro[3.3]heptan-6-yl]methyl]-5-(trifluoromethyl)-2-pyridone;
bis[6-(3-cyclopropyl-1H-1,2,4-triazol-5-yl)-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[3-[4-[5-methyl-3-(trifluoromethyl)pyrazol-1-yl]phenyl]azetidin-1-yl]methanone;
1-[[2-[6-(3-cyclopropyl-1H-1,2,4-triazol-5-yl)-2-azaspiro[3.3]heptane-2-carbonyl]-2-azaspiro[3.3]heptan-6-ylidene]methyl]-5-(trifluoromethyl)-2-pyridone;
[6-(3-cyclopropyl-1H-1,2,4-triazol-5-yl)-2-azaspiro[3.3]heptan-2-yl]-[6-[[5-(trifluoromethyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[4-(trifluoromethyl)triazol-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[4-(trifluoromethyl)triazol-1-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-(4-triflylphenyl)-2-azaspiro[3.3]heptan-2-yl]methanone;
[2-(4-chloro-2-mesyl-benzyl)-6-azaspiro[3.4]octan-6-yl]-[6-(5-cyclopropyl-4H-1,2,4-triazol-3-yl)-2-azaspiro[3.3]heptan-2-yl]methanone;
[6-[(2-tert-butylthiazol-5-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[4-[5-chloro-1-(2-hydroxy-2-methyl-propyl)indazol-3-yl]piperidino]-[6-(5-cyclopropyl-4H-1,2,4-triazol-3-yl)-2-azaspiro[3.3]heptan-2-yl]methanone;
1-[[2-[6-[3-(1-hydroxycyclopropyl)-1H-1,2,4-triazol-5-yl]-2-azaspiro[3.3]heptane-2-carbonyl]-2-azaspiro[3.3]heptan-6-yl]methyl]-5-(trifluoromethyl)-2-pyridone;
[6-(3-cyclopropyl-1H-1,2,4-triazol-5-yl)-2-azaspiro[3.3]heptan-2-yl]-[4-[3-[5-[1-(trifluoromethyl)cyclopropyl]-1,3,4-oxadiazol-2-yl]-1-bicyclo[1.1.1]pentanyl]piperidino]methanone;
[6-[3-(1-hydroxycyclopropyl)-1H-1,2,4-triazol-5-yl]-2-azaspiro[3.3]heptan-2-yl]-[3-[3-(4-triflylphenyl)-1-bicyclo[1.1.1]pentanyl]azetidin-1-yl]methanone;
[6-(3-cyclopropyl-1H-1,2,4-triazol-5-yl)-2-azaspiro[3.3]heptan-2-yl]-[6-(4-triflylbenzyl)-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[7-[3-(trifluoromethyl)phenyl]sulfonyl-2-azaspiro[3.5]nonan-2-yl]methanone;
[6-(3-cyclopropyl-1H-1,2,4-triazol-5-yl)-2-azaspiro[3.3]heptan-2-yl]-[3-[3-[5-[1-(trifluoromethyl)cyclopropyl]-1,3,4-oxadiazol-2-yl]-1-bicyclo[1.1.1]pentanyl]azetidin-1-yl]methanone;
[6-(3-cyclopropyl-1H-1,2,4-triazol-5-yl)-2-azaspiro[3.3]heptan-2-yl]-[6-[4-(trifluoromethyl)thiazol-2-yl]oxy-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[5-(trifluoromethyl)pyrazin-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[1-methyl-3-(trifluoromethyl)pyrazol-4-yl]oxy-2-azaspiro[3.3]heptan-2-yl]methanone;
4-[[2-[6-(3-cyclopropyl-1H-1,2,4-triazol-5-yl)-2-azaspiro[3.3]heptane-2-carbonyl]-2,6-diazaspiro[3.3]heptan-6-yl]methyl]-1-(2,2,2-trifluoroethyl)-2-pyridone;
[6-[3-(1-fluorocyclopropyl)-1H-1,2,4-triazol-5-yl]-2-azaspiro[3.3]heptan-2-yl]-[6-(4-triflylbenzyl)-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[3-methyl-5-(trifluoromethyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[7-[[4-(trifluoromethyl)phenyl]sulfonimidoyl]-2-azaspiro[3.5]nonan-2-yl]methanone;
[6-(3-cyclopropyl-1H-1,2,4-triazol-5-yl)-2-azaspiro[3.3]heptan-2-yl]-[6-[[4-(trifluoromethyl)thiazol-2-yl]methyl]-2,6-diazaspiro[3.3]heptan-2-yl]methanone;
[6-[3-(1-fluorocyclopropyl)-1H-1,2,4-triazol-5-yl]-2-azaspiro[3.3]heptan-2-yl]-[3-[5-[[1-(trifluoromethyl)cyclopropyl]methoxy]pyrazin-2-yl]azetidin-1-yl]methanone;
[6-(3-cyclopropyl-1H-1,2,4-triazol-5-yl)-2-azaspiro[3.3]heptan-2-yl]-[7-[[5-(trifluoromethyl)isothiazol-3-yl]methyl]-2,7-diazaspiro[3.5]nonan-2-yl]methanone;
5-[[2-[6-[5-(1-hydroxycyclopropyl)-4H-1,2,4-triazol-3-yl]-2-azaspiro[3.3]heptane-2-carbonyl]-2-azaspiro[3.3]heptan-6-yl]methyl]-2-(trifluoromethyl)isonicotinonitrile;
[6-(3-cyclopropyl-1H-1,2,4-triazol-5-yl)-2-azaspiro[3.3]heptan-2-yl]-[6-[(5-fluoro-2-pyridyl)oxy]-2-azaspiro[3.3]heptan-2-yl]methanone;
[6-(3-cyclopropyl-1H-1,2,4-triazol-5-yl)-2-azaspiro[3.3]heptan-2-yl]-[7-[3-(trifluoromethyl)phenyl]sulfonyl-2-azaspiro[3.5]nonan-2-yl]methanone;
[6-(3-cyclopropyl-1H-1,2,4-triazol-5-yl)-2-azaspiro[3.3]heptan-2-yl]-[7-[[5-(trifluoromethyl)-4H-1,2,4-triazol-3-yl]methyl]-2-azaspiro[3.5]nonan-2-yl]methanone;
[6-(3-cyclopropyl-1H-1,2,4-triazol-5-yl)-2-azaspiro[3.3]heptan-2-yl]-[6-[[1-methyl-5-(trifluoromethyl)triazol-4-yl]methyl]-2,6-diazaspiro[3.3]heptan-2-yl]methanone;
[3-[3-[[2-fluoro-4-(trifluoromethyl)phenyl]methyl]-1-bicyclo[1.1.1]pentanyl]azetidin-1-yl]-[6-[3-(1-hydroxycyclopropyl)-1H-1,2,4-triazol-5-yl]-2-azaspiro[3.3]heptan-2-yl]methanone;
[6-(3-cyclopropyl-1H-1,2,4-triazol-5-yl)-2-azaspiro[3.3]heptan-2-yl]-[4-[3-(5-neopentyl-1,3,4-oxadiazol-2-yl)-1-bicyclo[1.1.1]pentanyl]piperidino]methanone;
[6-(3-cyclopropyl-1H-1,2,4-triazol-5-yl)-2-azaspiro[3.3]heptan-2-yl]-[6-[[3-methyl-1-(2,2,2-trifluoroethyl)pyrazol-4-yl]methyl]-2,6-diazaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[3-(trifluoromethyl)-1,2,4-triazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-(2,2,2-trifluoroethyl)triazol-4-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[3-[5-[[[1-(trifluoromethyl);
cyclopropyl]amino]methyl]pyrazin-2-yl]azetidin-1-yl]methanone;
[6-(3-cyclopropyl-1H-1,2,4-triazol-5-yl)-2-azaspiro[3.3]heptan-2-yl]-[6-[[5-(trifluoromethyl)-1,2,4-triazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-methyl-3-(trifluoromethyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[difluoro-[6-(trifluoromethyl)pyridazin-3-yl]methyl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[3-[3-[5-[1-(trifluoromethyl)cyclopropyl]-1,3,4-thiadiazol-2-yl]-1-bicyclo[1.1.1]pentanyl]azetidin-1-yl]methanone;
[6-[difluoro-[6-(trifluoromethyl)pyridazin-3-yl]methyl]-2-azaspiro[3.3]heptan-2-yl]-[6-[3-(1-hydroxycyclopropyl)-1H-1,2,4-triazol-5-yl]-2-azaspiro[3.3]heptan-2-yl]methanone;
[6-(5-cyclopropyl-4H-1,2,4-triazol-3-yl)-2-azaspiro[3.3]heptan-2-yl]-[3-[4-(5-ethoxy-3-methyl-pyrazol-1-yl)phenyl]azetidin-1-yl]methanone;
[6-(3-cyclopropyl-1H-1,2,4-triazol-5-yl)-2-azaspiro[3.3]heptan-2-yl]-[6-[[5-(trifluoromethyl)-1,2,4-thiadiazol-3-yl]methyl]-2-azaspiro[3.3]heptan-2-yl]methanone;
[6-(2-fluoro-4-triflyl-benzyl)-2,6-diazaspiro[3.3]heptan-2-yl]-[6-[3-(1-hydroxycyclopropyl)-1H-1,2,4-triazol-5-yl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[6-(trifluoromethyl)pyridazin-3-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[2-(trifluoromethyl)imidazol-1-yl]methyl]-2-azaspiro[3.3]heptan-2-yl]methanone;
[3-[4-(4-chloro-2-mesyl-phenyl)phenyl]azetidin-1-yl]-[6-(1H-1,2,4-triazol-5-yl)-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[3-fluoro-5-(trifluoromethyl)benzyl]-2-azaspiro[3.3]heptan-2-yl]methanone;
[6-(3-cyclopropyl-1H-1,2,4-triazol-5-yl)-2-azaspiro[3.3]heptan-2-yl]-[7-[[3-(trifluoromethyl)phenyl]sulfonimidoyl]-2-azaspiro[3.5]nonan-2-yl]methanone;
[6-[3-(1-fluorocyclopropyl)-1H-1,2,4-triazol-5-yl]-2-azaspiro[3.3]heptan-2-yl]-[6-[[5-(trifluoromethyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-methyl-4-(trifluoromethyl)thiazol-2-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[3-[3-[3-(trifluoromethoxy)phenyl]-1-bicyclo[1.1.1]pentanyl]azetidin-1-yl]methanone;
[6-[3-(1-fluorocyclopropyl)-1H-1,2,4-triazol-5-yl]-2-azaspiro[3.3]heptan-2-yl]-[6-[[6-(trifluoromethyl)pyridazin-3-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[7-[[2-methyl-4-(trifluoromethyl)pyrazol-3-yl]methyl]-2,7-diazaspiro[3.5]nonan-2-yl]methanone;
[6-(3-cyclopropyl-1H-1,2,4-triazol-5-yl)-2-azaspiro[3.3]heptan-2-yl]-[6-(4-dimethylphosphoryl-2-fluoro-benzyl)-2-azaspiro[3.3]heptan-2-yl]methanone;
2-[3-[1-[6-(3-cyclopropyl-1H-1,2,4-triazol-5-yl)-2-azaspiro[3.3]heptane-2-carbonyl]azetidin-3-yl]-1-bicyclo[1.1.1]pentanyl]-5-fluoro-benzonitrile;
3-[[2-[6-[3-(1-fluorocyclopropyl)-1H-1,2,4-triazol-5-yl]-2-azaspiro[3.3]heptane-2-carbonyl]-2-azaspiro[3.3]heptan-6-yl]methyl]-5-(trifluoromethyl)benzonitrile;
[6-[3-(3,3-difluorocyclobutyl)-1H-1,2,4-triazol-5-yl]-2-azaspiro[3.3]heptan-2-yl]-[6-[[5-(trifluoromethyl)pyrazin-2-yl]methyl]-2-azaspiro[3.3]heptan-2-yl]methanone;
[3-[3-[5-(2,2-dimethylpropyl)-1,3,4-thiadiazol-2-yl]-1-bicyclo[1.1.1];
pentanyl]azetidin-1-yl]-[6-[3-(1-hydroxycyclopropyl)-1H-1,2,4-triazol-5-yl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[2-fluoro-4-(trifluoromethyl)benzyl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[4-fluoro-2-(trifluoromethyl)benzyl]-2-azaspiro[3.3]heptan-2-yl]methanone;
[6-(3-cyclopropyl-1H-1,2,4-triazol-5-yl)-2-azaspiro[3.3]heptan-2-yl]-[3-[5-[[1-(trifluoromethyl)cyclopropyl]methoxy]pyrazin-2-yl]azetidin-1-yl]methanone;
[6-[3-(1-fluorocyclopropyl)-1H-1,2,4-triazol-5-yl]-2-azaspiro[3.3]heptan-2-yl]-[3-(2-fluoro-4-triflyl-benzyl)oxyazetidin-1-yl]methanone;
[6-[3-(1-hydroxycyclopropyl)-1H-1,2,4-triazol-5-yl]-2-azaspiro[3.3]heptan-2-yl]-[3-[5-[1-(trifluoromethyl)cyclopropyl]-2-pyridyl]azetidin-1-yl]methanone;
[6-(3-cyclopropyl-1H-1,2,4-triazol-5-yl)-2-azaspiro[3.3]heptan-2-yl]-[6-[[1-methyl-3-(trifluoromethyl)pyrazol-4-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[1-methyl-3-(trifluoromethyl)pyrazol-4-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[3-[[5-(trifluoromethyl)pyrazin-2-yl]amino]-1-bicyclo[1.1.1]pentanyl]methanone;
[6-[3-(1-aminocyclopropyl)-1H-1,2,4-triazol-5-yl]-2-azaspiro[3.3]heptan-2-yl]-[6-[[4-(trifluoromethyl-sulfonimidoyl)phenyl]methyl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[1-methyl-5-(trifluoromethyl)pyrazol-4-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[3-[(8 S)-8-methyl-8-oxo-8λ⁶-thia-9-azatricyclo[8.4.0.02,7]tetradeca-1(10),2(7),3,5,8,11,13-heptaen-12-yl]azetidin-1-yl]methanone;
2-[[2-[6-[5-(1-hydroxycyclopropyl)-4H-1,2,4-triazol-3-yl]-2-azaspiro[3.3]heptane-2-carbonyl]-2-azaspiro[3.3]heptan-6-yl]methyl]-5-(trifluoromethyl)benzonitrile;
[6-(5-cyclopropyl-4H-1,2,4-triazol-3-yl)-2-azaspiro[3.3]heptan-2-yl]-[3-[(8R)-8-methyl-8-oxo-8λ⁶-thia-9-azatricyclo[8.4.0.02,7]tetradeca-1(10),2(7),3,5,8,11,13-heptaen-12-yl]azetidin-1-yl]methanone;
[6-(3-cyclopropyl-1H-1,2,4-triazol-5-yl)-2-azaspiro[3.3]heptan-2-yl]-[6-[[1-methyl-4-(trifluoromethyl)pyrazol-3-yl]methyl]-2-azaspiro[3.3]heptan-2-yl]methanone;
1-[[2-[6-(3-cyclopropyl-1H-1,2,4-triazol-5-yl)-2-azaspiro[3.3]heptane-2-carbonyl]-2-azaspiro[3.3]heptan-6-yl]methyl]-6-keto-nicotinonitrile;
[6-(3-cyclopropyl-1H-1,2,4-triazol-5-yl)-2-azaspiro[3.3]heptan-2-yl]-[6-[2-(methylsulfonimidoyl)-4-(trifluoromethyl)benzyl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[7-(3-triflylbenzyl)-2,7-diazaspiro[3.4]octan-2-yl]methanone;
[6-[(4-tert-butyloxazol-2-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[3-(trifluoromethoxy)phenyl]sulfonyl-2-azaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethyl)-1,3,4-thiadiazol-2-yl]methyl]-2,6-diazaspiro[3.3]heptan-2-yl]methanone;
4-[[2-[6-[3-(3,3-difluorocyclobutyl)-1H-1,2,4-triazol-5-yl]-2-azaspiro[3.3]heptane-2-carbonyl]-2-azaspiro[3.3]heptan-6-yl]methyl]-3-fluoro-benzonitrile;
[6-[(5-tert-butyl-1,3,4-thiadiazol-2-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-[(4-cyclopropylthiadiazol-5-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[4-(trifluoromethyl)thiazol-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[3-[3-[4-(trifluoromethoxy)phenyl]-1-bicyclo[1.1.1]pentanyl]azetidin-1-yl]methanone;
[6-[3-(1-hydroxycyclopropyl)-1H-1,2,4-triazol-5-yl]-2-azaspiro[3.3]heptan-2-yl]-[6-[[1-methyl-5-(trifluoromethyl)pyrazol-3-yl]methyl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[3-[3-(5-neopentyl-1,3,4-thiadiazol-2-yl)-1-bicyclo[1.1.1]pentanyl]azetidin-1-yl]methanone;
[6-[3-(3,3-difluorocyclobutyl)-1H-1,2,4-triazol-5-yl]-2-azaspiro[3.3]heptan-2-yl]-[6-[3-(methylsulfonimidoyl)benzyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethyl)-1H-1,2,4-triazol-3-yl]methyl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[5-(difluoromethyl)-1H-pyrazol-3-yl]methyl]-2-azaspiro[3.3]heptan-2-yl]methanone;
[3-[4-(cyclopropylsulfonimidoyl);
phenyl]azetidin-1-yl]-[6-[3-(1-hydroxycyclopropyl)-1H-1,2,4-triazol-5-yl]-2-azaspiro[3.3]heptan-2-yl]methanone;
[6-(5-cyclopropyl-4H-1,2,4-triazol-3-yl)-2-azaspiro[3.3]heptan-2-yl]-[6-(5-fluoro-3-pyridyl)-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[4-[3-(5-neopentyl-1,3,4-oxadiazol-2-yl)-1-bicyclo[1.1.1]pentanyl]piperidino]methanone;
5-[[2-[6-(3-cyclopropyl-1H-1,2,4-triazol-5-yl)-2-azaspiro[3.3]heptane-2-carbonyl]-2-azaspiro[3.3]heptan-6-yl]oxy]-2-(trifluoromethyl)benzonitrile;
[6-(5-cyclopropyl-4H-1,2,4-triazol-3-yl)-2-azaspiro[3.3]heptan-2-yl]-[3-[4-(4-mesylpyrazol-1-yl)phenyl]azetidin-1-yl]methanone;
[6-(3-cyclopropyl-1H-1,2,4-triazol-5-yl)-2-azaspiro[3.3]heptan-2-yl]-[6-[[6-(trifluoromethoxy)-3-pyridyl]methyl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[3-(2-fluoro-4-triflyl-benzyl)oxyazetidin-1-yl]methanone;
[6-[3-(1-fluorocyclopropyl)-1H-1,2,4-triazol-5-yl]-2-azaspiro[3.3]heptan-2-yl]-[3-[5-[[1-(trifluoromethyl);
cyclopropyl]methylamino]pyrazin-2-yl]azetidin-1-yl]methanone;
[6-(3-cyclopropyl-1H-1,2,4-triazol-5-yl)-2-azaspiro[3.3]heptan-2-yl]-[6-[[2-methyl-4-(trifluoromethyl)thiazol-5-yl]methyl]-2,6-diazaspiro[3.3]heptan-2-yl]methanone;
[6-(4-cyclopropylsulfonylbenzyl)-2-azaspiro[3.3]heptan-2-yl]-[6-[3-(1-hydroxycyclopropyl)-1H-1,2,4-triazol-5-yl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-methyl-5-(trifluoromethyl)pyrazol-3-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[7-[[5-(difluoromethyl)thiazol-2-yl]methyl]-2,7-diazaspiro[3.5]nonan-2-yl]methanone;
[6-[3-(3,3-difluorocyclobutyl)-1H-1,2,4-triazol-5-yl]-2-azaspiro[3.3]heptan-2-yl]-[6-[[3-fluoro-5-(trifluoromethyl)-2-pyridyl]methyl]-2-azaspiro[3.3]heptan-2-yl]methanone;
5-fluoro-2-[3-[1-[6-[3-(1-hydroxycyclopropyl)-1H-1,2,4-triazol-5-yl]-2-azaspiro[3.3]heptane-2-carbonyl]azetidin-3-yl]-1-bicyclo[1.1.1]pentanyl]benzonitrile;
1-[[2-[6-(3-cyclopropyl-1H-1,2,4-triazol-5-yl)-2-azaspiro[3.3]heptane-2-carbonyl]-2-azaspiro[3.3]heptan-6-yl]methyl]-2-keto-nicotinonitrile;
[6-[3-(3,3-difluorocyclobutyl)-1H-1,2,4-triazol-5-yl]-2-azaspiro[3.3]heptan-2-yl]-[6-[[1-methyl-5-(trifluoromethyl)pyrazol-3-yl]methyl]-2-azaspiro[3.3]heptan-2-yl]methanone;
5-[[2-[6-[3-(1-fluorocyclopropyl)-1H-1,2,4-triazol-5-yl]-2-azaspiro[3.3]heptane-2-carbonyl]-2-azaspiro[3.3]heptan-6-yl]methyl]-3-(trifluoromethyl)picolinonitrile;
[6-(3-cyclopropyl-1H-1,2,4-triazol-5-yl)-2-azaspiro[3.3]heptan-2-yl]-[6-[[1-(trifluoromethyl)pyrazol-4-yl]methyl]-2,6-diazaspiro[3.3]heptan-2-yl]methanone;
[3-[3-(3-cyclopropyl-1H-pyrazol-5-yl)-1-bicyclo[1.1.1]pentanyl]azetidin-1-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethyl)-1H-pyrazol-4-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[3-[4-(2-mesylphenyl)phenyl]azetidin-1-yl]methanone;
[6-[3-(1-fluorocyclopropyl)-1H-1,2,4-triazol-5-yl]-2-azaspiro[3.3]heptan-2-yl]-[6-[[5-(trifluoromethyl)-2-pyridyl]methyl]-2-azaspiro[3.3]heptan-2-yl]methanone;
[6-(3-fluoro-5-triflyl-benzyl)-2-azaspiro[3.3]heptan-2-yl]-[6-[3-(1-hydroxycyclopropyl)-1H-1,2,4-triazol-5-yl]-2-azaspiro[3.3]heptan-2-yl]methanone;
[6-(5-cyclopropyl-4H-1,2,4-triazol-3-yl)-2-azaspiro[3.3]heptan-2-yl]-[4-[1-(2-hydroxyethyl)-5-(trifluoromethyl)indazol-3-yl]piperidino]methanone;
[6-(3-cyclopropyl-1H-1,2,4-triazol-5-yl)-2-azaspiro[3.3]heptan-2-yl]-[6-[[5-(trifluoromethyl)-3-pyridyl]oxy]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-(2,2,2-trifluoroethyl)tetrazol-5-yl]methyl]-2,6-diazaspiro[3.3]heptan-2-yl]methanone;
[6-[3-(1-fluorocyclopropyl)-1H-1,2,4-triazol-5-yl]-2-azaspiro[3.3]heptan-2-yl]-[6-[2-fluoro-4-(trifluoromethyl)benzyl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[4-(trifluoromethyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[4-(trifluoromethyl)-1H-imidazol-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[7-[[5-(trifluoromethyl)-1H-pyrazol-3-yl]methyl]-2,7-diazaspiro[3.5]nonan-2-yl]methanone;
[6-[3-(3,3-difluorocyclobutyl)-1H-1,2,4-triazol-5-yl]-2-azaspiro[3.3]heptan-2-yl]-[6-(4-triflylbenzyl)-2-azaspiro[3.3]heptan-2-yl]methanone;
[6-(3-cyclopropyl-1H-1,2,4-triazol-5-yl)-2-azaspiro[3.3]heptan-2-yl]-[3-[3-[[2-fluoro-4-(trifluoromethyl);
phenyl]methyl]-1-bicyclo[1.1.1];
pentanyl]azetidin-1-yl]methanone;
[6-(3-cyclopropyl-1H-1,2,4-triazol-5-yl)-2-azaspiro[3.3]heptan-2-yl]-[3-[3-[[1-(trifluoromethyl)cyclopropyl];
methylsulfonyl]-1-bicyclo[1.1.1];
pentanyl]azetidin-1-yl]methanone;
[6-[3-(1-hydroxycyclopropyl)-1H-1,2,4-triazol-5-yl]-2-azaspiro[3.3]heptan-2-yl]-[3-[3-[3-(trifluoromethoxy)phenyl]-1-bicyclo[1.1.1]pentanyl]azetidin-1-yl]methanone;
[6-[3-(3,3-difluorocyclobutyl)-1H-1,2,4-triazol-5-yl]-2-azaspiro[3.3]heptan-2-yl]-[6-[[1-methyl-5-(trifluoromethyl)pyrazol-4-yl]methyl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[3-(trifluoromethyl)isothiazol-5-yl]methyl]-2,6-diazaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[3-(trifluoromethylsulfonimidoyl)benzyl]-2-azaspiro[3.3]heptan-2-yl]methanone;
[6-(3-cyclopropyl-1H-1,2,4-triazol-5-yl)-2-azaspiro[3.3]heptan-2-yl]-[6-[3-(trifluoromethoxy)phenyl]sulfonyl-2-azaspiro[3.3]heptan-2-yl]methanone;
[6-(5-cyclopropyl-4H-1,2,4-triazol-3-yl)-2-azaspiro[3.3]heptan-2-yl]-[3-[4-[3-(difluoromethyl)-5-methyl-1,2,4-triazol-1-yl]phenyl]azetidin-1-yl]methanone;
5-[[2-[6-(5-cyclopropyl-4H-1,2,4-triazol-3-yl)-2-azaspiro[3.3]heptane-2-carbonyl]-2-azaspiro[3.3]heptan-6-yl]methyl]-3-(trifluoromethyl)picolinonitrile;
[6-[3-(1-hydroxycyclopropyl)-1H-1,2,4-triazol-5-yl]-2-azaspiro[3.3]heptan-2-yl]-[6-[[4-(trifluoromethyl)thiazol-2-yl]methyl]-2-azaspiro[3.3]heptan-2-yl]methanone;
5-[[2-[6-(3-cyclopropyl-1H-1,2,4-triazol-5-yl)-2-azaspiro[3.3]heptane-2-carbonyl]-2,6-diazaspiro[3.3]heptan-6-yl]methyl]-1-methyl-imidazole-4-carbonitrile;
[2-(4-chloro-2-mesyl-phenyl)-6-azaspiro[3.4]octan-6-yl]-[6-(5-cyclopropyl-4H-1,2,4-triazol-3-yl)-2-azaspiro[3.3]heptan-2-yl]methanone;
[6-(3-cyclopropyl-1H-1,2,4-triazol-5-yl)-2-azaspiro[3.3]heptan-2-yl]-[7-[[2-methyl-5-(trifluoromethyl)pyrazol-3-yl]methyl]-2,7-diazaspiro[3.5]nonan-2-yl]methanone;
[6-(3-cyclopropyl-1H-1,2,4-triazol-5-yl)-2-azaspiro[3.3]heptan-2-yl]-[3-[3-(4-triflylphenyl)-1-bicyclo[1.1.1]pentanyl]azetidin-1-yl]methanone;
5-[[2-[6-(3-cyclopropyl-1H-1,2,4-triazol-5-yl)-2-azaspiro[3.3]heptane-2-carbonyl]-2,6-diazaspiro[3.3]heptan-6-yl]methyl]-2-pyridone;
[6-(3-cyclopropyl-1H-1,2,4-triazol-5-yl)-2-azaspiro[3.3]heptan-2-yl]-[6-(1-methylpyrazol-4-yl)oxy-2-azaspiro[3.3]heptan-2-yl]methanone;
[6-(3-cyclopropyl-1H-1,2,4-triazol-5-yl)-2-azaspiro[3.3]heptan-2-yl]-[(6S)-6-(3-triflylbenzyl)-2-azaspiro[3.4]octan-2-yl]methanone;
[6-(3-cyclopropyl-1H-1,2,4-triazol-5-yl)-2-azaspiro[3.3]heptan-2-yl]-[7-[[5-(trifluoromethyl)isoxazol-3-yl]methyl]-2,7-diazaspiro[3.5]nonan-2-yl]methanone;
[6-[3-(3,3-difluorocyclobutyl)-1H-1,2,4-triazol-5-yl]-2-azaspiro[3.3]heptan-2-yl]-[6-[[5-(trifluoromethyl)-3-pyridyl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[2-(2,2,2-trifluoroethyl)tetrazol-5-yl]methyl]-2,6-diazaspiro[3.3]heptan-2-yl]methanone;
5-[[2-[6-[3-(3,3-difluorocyclobutyl)-1H-1,2,4-triazol-5-yl]-2-azaspiro[3.3]heptane-2-carbonyl]-2-azaspiro[3.3]heptan-6-yl]methyl]-2-(trifluoromethyl)benzonitrile;
[6-[3-(1-fluorocyclopropyl)-1H-1,2,4-triazol-5-yl]-2-azaspiro[3.3]heptan-2-yl]-[6-[[4-(trifluoromethyl)triazol-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[7-(3-triflylbenzyl)-2,7-diazaspiro[3.4]octan-2-yl]methanone;
[6-[3-(1-hydroxycyclopropyl)-1H-1,2,4-triazol-5-yl]-2-azaspiro[3.3]heptan-2-yl]-[7-[4-(trifluoromethyl)phenyl]sulfonyl-2-azaspiro[3.5]nonan-2-yl]methanone;
[6-[3-(1-hydroxycyclopropyl)-1H-1,2,4-triazol-5-yl]-2-azaspiro[3.3]heptan-2-yl]-[3-[3-[4-(trifluoromethoxy)phenyl]-1-bicyclo[1.1.1]pentanyl]azetidin-1-yl]methanone;
[6-[3-(1-hydroxycyclopropyl)-1H-1,2,4-triazol-5-yl]-2-azaspiro[3.3]heptan-2-yl]-[3-[3-[5-(trifluoromethyl)pyrazin-2-yl]oxy-1-bicyclo[1.1.1]pentanyl]azetidin-1-yl]methanone;
[6-(3-cyclopropyl-1H-1,2,4-triazol-5-yl)-2-azaspiro[3.3]heptan-2-yl]-[6-[[2-ethyl-5-(trifluoromethyl)pyrazol-3-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-(2-fluoro-4-triflyl-benzyl)-2,6-diazaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[4-(trifluoromethylsulfonimidoyl)benzyl]-2-azaspiro[3.3]heptan-2-yl]methanone;
2-[[2-[6-(5-cyclopropyl-4H-1,2,4-triazol-3-yl)-2-azaspiro[3.3]heptane-2-carbonyl]-2-azaspiro[3.3]heptan-6-yl]methyl]-5-(trifluoromethyl)benzonitrile;
[6-(3-cyclopropyl-1H-1,2,4-triazol-5-yl)-2-azaspiro[3.3]heptan-2-yl]-[3-[3-[5-[1-(trifluoromethyl)cyclopropyl]-1,3,4-thiadiazol-2-yl]-1-bicyclo[1.1.1]pentanyl]azetidin-1-yl]methanone;
[6-[3-(1-hydroxycyclopropyl)-1H-1,2,4-triazol-5-yl]-2-azaspiro[3.3]heptan-2-yl]-[6-[4-(trifluoromethylsulfonimidoyl)benzyl]-2-azaspiro[3.3]heptan-2-yl]methanone;
[6-(5-cyclopropyl-4H-1,2,4-triazol-3-yl)-2-azaspiro[3.3]heptan-2-yl]-[3-[4-(3-ethoxy-5-methyl-pyrazol-1-yl)phenyl]azetidin-1-yl]methanone;
[6-[3-(1-hydroxycyclopropyl)-1H-1,2,4-triazol-5-yl]-2-azaspiro[3.3]heptan-2-yl]-[6-[(5-triflyl-3-pyridyl)methyl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[3-(trifluoromethyl)isothiazol-5-yl]methyl]-2,6-diazaspiro[3.3]heptan-2-yl]methanone;
[3-[3-(4-chloro-2-mesyl-phenyl)-1-bicyclo[1.1.1]pentanyl]azetidin-1-yl]-[6-(5-cyclopropyl-4H-1,2,4-triazol-3-yl)-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[3-[4-[1-(trifluoromethyl)cyclopropyl]phenyl]azetidin-1-yl]methanone;
[6-[3-(1-fluorocyclopropyl)-1H-1,2,4-triazol-5-yl]-2-azaspiro[3.3]heptan-2-yl]-[3-[4-(trifluoromethylsulfonimidoyl)phenyl]azetidin-1-yl]methanone;
[6-[3-(1-fluorocyclopropyl)-1H-1,2,4-triazol-5-yl]-2-azaspiro[3.3]heptan-2-yl]-[6-[[2-(trifluoromethyl)pyrimidin-5-yl]methyl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[6-(trifluoromethoxy)-3-pyridyl]methyl]-2-azaspiro[3.3]heptan-2-yl]methanone;
1-[[2-[6-[3-(3,3-difluorocyclobutyl)-1H-1,2,4-triazol-5-yl]-2-azaspiro[3.3]heptane-2-carbonyl]-2-azaspiro[3.3]heptan-6-yl]methyl]pyrazole-4-carbonitrile;
[6-(3-cyclopropyl-1H-1,2,4-triazol-5-yl)-2-azaspiro[3.3]heptan-2-yl]-[6-[[1-(2,2,2-trifluoroethyl)pyrazol-4-yl]methyl]-2,6-diazaspiro[3.3]heptan-2-yl]methanone;
4-[[2-[6-[3-(1-fluorocyclopropyl)-1H-1,2,4-triazol-5-yl]-2-azaspiro[3.3]heptane-2-carbonyl]-2-azaspiro[3.3]heptan-6-yl]methyl]-2-(trifluoromethyl)benzonitrile;
[6-[3-(1-hydroxycyclopropyl)-1H-1,2,4-triazol-5-yl]-2-azaspiro[3.3]heptan-2-yl]-[3-[3-[[6-(trifluoromethyl)-3-pyridyl]methyl]-1-bicyclo[1.1.1]pentanyl]azetidin-1-yl]methanone;
[6-(3-cyclopropyl-1H-1,2,4-triazol-5-yl)-2-azaspiro[3.3]heptan-2-yl]-[6-[[1-(2,2,2-trifluoroethyl)-3-(trifluoromethyl)pyrazol-4-yl]methyl]-2-azaspiro[3.3]heptan-2-yl]methanone;
3-[[2-[6-(3-cyclopropyl-1H-1,2,4-triazol-5-yl)-2-azaspiro[3.3]heptane-2-carbonyl]-2,6-diazaspiro[3.3]heptan-6-yl]methyl]-1-(2,2,2-trifluoroethyl)-2-pyridone;
[6-[3-(1-hydroxycyclopropyl)-1H-1,2,4-triazol-5-yl]-2-azaspiro[3.3]heptan-2-yl]-[3-[3-[5-[1-(trifluoromethyl)cyclopropyl]-1,3,4-oxadiazol-2-yl]-1-bicyclo[1.1.1]pentanyl]azetidin-1-yl]methanone;
[6-[3-(3,3-difluorocyclobutyl)-1H-1,2,4-triazol-5-yl]-2-azaspiro[3.3]heptan-2-yl]-[7-[[5-(trifluoromethyl)-2-pyridyl]methyl]-2-azaspiro[3.5]nonan-2-yl]methanone;
[6-(5-cyclopropyl-4H-1,2,4-triazol-3-yl)-2-azaspiro[3.3]heptan-2-yl]-[6-[[3-(trifluoromethoxy)phenyl]sulfonimidoyl]-2-azaspiro[3.3]heptan-2-yl]methanone;
[3-[2-fluoro-4-(trifluoromethyl-sulfonimidoyl)benzyl]oxyazetidin-1-yl]-[6-[3-(1-hydroxycyclopropyl)-1H-1,2,4-triazol-5-yl]-2-azaspiro[3.3]heptan-2-yl]methanone;
[2-(4-chloro-2-mesyl-phenyl)-6-azaspiro[3.4]octan-6-yl]-[6-(5-cyclopropyl-4H-1,2,4-triazol-3-yl)-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[3-[3-[[1-(trifluoromethyl)-cyclopropyl]methylamino]-1-bicyclo[1.1.1]pentanyl]azetidin-1-yl]methanone;
3-[[2-[6-[3-(3,3-difluorocyclobutyl)-1H-1,2,4-triazol-5-yl]-2-azaspiro[3.3]heptane-2-carbonyl]-2-azaspiro[3.3]heptan-6-yl]methyl]-5-(trifluoromethyl)benzonitrile;
4-[[2-[6-(3-cyclopropyl-1H-1,2,4-triazol-5-yl)-2-azaspiro[3.3]heptane-2-carbonyl]-2,6-diazaspiro[3.3]heptan-6-yl]methyl]-2-methyl-pyrazole-3-sulfonamide;
[6-(3-cyclopropyl-1H-1,2,4-triazol-5-yl)-2-azaspiro[3.3]heptan-2-yl]-[3-[3-[5-(2,2-dimethylpropyl)-1,3,4-thiadiazol-2-yl]-1-bicyclo[1.1.1]-pentanyl]azetidin-1-yl]methanone;
[6-[3-(3,3-difluorocyclobutyl)-1H-1,2,4-triazol-5-yl]-2-azaspiro[3.3]heptan-2-yl]-[6-(4-mesylbenzyl)-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[3-[3-(5-neopentyl-1,3,4-thiadiazol-2-yl)-1-bicyclo[1.1.1]pentanyl]azetidin-1-yl]methanone;
[6-(3-cyclopropyl-1H-1,2,4-triazol-5-yl)-2-azaspiro[3.3]heptan-2-yl]-[6-[2-fluoro-4-(trifluoromethyl)benzyl]-2,6-diazaspiro[3.3]heptan-2-yl]methanone;
5-[[2-[6-[3-(1-fluorocyclopropyl)-1H-1,2,4-triazol-5-yl]-2-azaspiro[3.3]heptane-2-carbonyl]-2-azaspiro[3.3]heptan-6-yl]methyl]-2-(trifluoromethyl)benzonitrile;
[6-(2,4-difluorobenzyl)-2-azaspiro[3.3]heptan-2-yl]-[6-[3-(3,3-difluorocyclobutyl)-1H-1,2,4-triazol-5-yl]-2-azaspiro[3.3]heptan-2-yl]methanone;
[3-[3-(5-cyclopropyl-3-methyl-pyrazol-1-yl)-1-bicyclo[1.1.1]pentanyl]azetidin-1-yl]-[6-(3-cyclopropyl-1H-1,2,4-triazol-5-yl)-2-azaspiro[3.3]heptan-2-yl]methanone;
[3-[3-(5-cyclopropyl-3-methyl-pyrazol-1-yl)-1-bicyclo[1.1.1]pentanyl]azetidin-1-yl]-[6-[3-(1-hydroxycyclopropyl)-1H-1,2,4-triazol-5-yl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[[3-(trifluoromethyl)-1H-pyrazol-5-yl]methyl]-2-azaspiro[3.3]heptan-2-yl]methanone;
4-[[2-[6-(3-cyclopropyl-1H-1,2,4-triazol-5-yl)-2-azaspiro[3.3]heptane-2-carbonyl]-2-azaspiro[3.3]heptan-6-yl]methyl]-2-(trifluoromethyl)benzonitrile;
[6-[(4-tert-butylisoxazol-3-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[1-methyl-5-(trifluoromethyl)pyrazol-4-yl]oxy-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[3-[3-[[5-(trifluoromethyl)pyrazin-2-yl]amino]-1-bicyclo[1.1.1]pentanyl]azetidin-1-yl]methanone;
[6-(3-cyclopropyl-1H-1,2,4-triazol-5-yl)-2-azaspiro[3.3]heptan-2-yl]-[(6R)-6-(3-triflylbenzyl)-2-azaspiro[3.4]octan-2-yl]methanone;
[6-(3-cyclopropyl-1H-1,2,4-triazol-5-yl)-2-azaspiro[3.3]heptan-2-yl]-[3-[3-(3-methyl-1H-pyrazol-5-yl)-1-bicyclo[1.1.1]pentanyl]azetidin-1-yl]methanone;
[6-[3-(3,3-difluorocyclobutyl)-1H-1,2,4-triazol-5-yl]-2-azaspiro[3.3]heptan-2-yl]-[6-[[5-(trifluoromethyl)thiazol-2-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[2-(2,2-difluoroethyl)pyrazol-3-yl]methyl]-2,6-diazaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[5-(trifluoromethyl)thiazol-2-yl]methyl]-2,6-diazaspiro[3.3]heptan-2-yl]methanone;
[6-[3-(1-hydroxycyclopropyl)-1H-1,2,4-triazol-5-yl]-2-azaspiro[3.3]heptan-2-yl]-[(6S)-6-(4-triflylbenzyl)-2-azaspiro[3.4]octan-2-yl]methanone;
[6-(5-cyclopropyl-4H-1,2,4-triazol-3-yl)-2-azaspiro[3.3]heptan-2-yl]-[3-[4-[5-(methylamino)-1,3,4-thiadiazol-2-yl]phenyl]azetidin-1-yl]methanone;
[6-(3-cyclopropyl-1H-1,2,4-triazol-5-yl)-2-azaspiro[3.3]heptan-2-yl]-[6-[[1-(trifluoromethyl)pyrazol-3-yl]methyl]-2,6-diazaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[1-methyl-5-(trifluoromethyl)pyrazol-3-yl]methyl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[4-(trifluoromethylsulfonimidoyl)benzyl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-(3-triflylbenzyl)-2-azaspiro[3.3]heptan-2-yl]methanone;
4-[[2-[6-(3-cyclopropyl-1H-1,2,4-triazol-5-yl)-2-azaspiro[3.3]heptane-2-carbonyl]-2-azaspiro[3.3]heptan-6-yl]oxy]-2-(trifluoromethyl)benzonitrile;
5-[[2-[6-[5-(1-hydroxycyclopropyl)-4H-1,2,4-triazol-3-yl]-2-azaspiro[3.3]heptane-2-carbonyl]-2-azaspiro[3.3]heptan-6-yl]methyl]-2-(trifluoromethyl)nicotinonitrile;
[6-(3-cyclopropyl-1H-1,2,4-triazol-5-yl)-2-azaspiro[3.3]heptan-2-yl]-[6-[[5-(trifluoromethyl)-1H-pyrazol-4-yl]methyl]-2-azaspiro[3.3]heptan-2-yl]methanone;
[6-[(4-cyclobutylthiadiazol-5-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethyl)isoxazol-3-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[5-(difluoromethyl)-1H-pyrazol-3-yl]methyl]-2-azaspiro[3.3]heptan-2-yl]methanone;
5-[[(6S)-2-[6-(5-cyclopropyl-4H-1,2,4-triazol-3-yl)-2-azaspiro[3.3]heptane-2-carbonyl]-2-azaspiro[3.4]octan-6-yl]methyl]-2-(trifluoromethyl)isonicotinonitrile;
[6-(3-cyclopropyl-1H-1,2,4-triazol-5-yl)-2-azaspiro[3.3]heptan-2-yl]-[(6S)-6-(4-triflylbenzyl)-2-azaspiro[3.4]octan-2-yl]methanone;
[6-[(5-chloro-3-fluoro-2-pyridyl)methyl]-2-azaspiro[3.3]heptan-2-yl]-[6-(3-cyclopropyl-1H-1,2,4-triazol-5-yl)-2-azaspiro[3.3]heptan-2-yl]methanone;
[6-[(5-cyclobutyl-1H-pyrazol-3-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-[[5-(difluoromethyl)-1H-pyrazol-3-yl]methyl]-2-azaspiro[3.3]heptan-2-yl]-[6-[3-(1-hydroxycyclopropyl)-1H-1,2,4-triazol-5-yl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[(5-triflyl-2-pyridyl)methyl]-2-azaspiro[3.3]heptan-2-yl]methanone;
[6-[(3-chloro-5-fluoro-2-pyridyl)methyl]-2-azaspiro[3.3]heptan-2-yl]-[6-[3-(3,3-difluorocyclobutyl)-1H-1,2,4-triazol-5-yl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-(2,2,2-trifluoroethyl)pyrazol-3-yl]methyl]-2,6-diazaspiro[3.3]heptan-2-yl]methanone;
[6-[(5-chloro-2-pyridyl)oxy]-2-azaspiro[3.3]heptan-2-yl]-[6-(3-cyclopropyl-1H-1,2,4-triazol-5-yl)-2-azaspiro[3.3]heptan-2-yl]methanone;
[6-[[3-fluoro-5-(trifluoromethyl)-2-pyridyl]methyl]-2-azaspiro[3.3]heptan-2-yl]-[6-(1H-1,2,4-triazol-5-yl)-2-azaspiro[3.3]heptan-2-yl]methanone;
1-[[2-[6-[3-(1-hydroxycyclopropyl)-1H-1,2,4-triazol-5-yl]-2-azaspiro[3.3]heptane-2-carbonyl]-2-azaspiro[3.3]heptan-6-yl]methyl]-5-(trifluoromethyl)-2-pyridone;
[6-[(3-chloro-5-fluoro-2-pyridyl)oxy]-2-azaspiro[3.3]heptan-2-yl]-[6-(3-cyclopropyl-1H-1,2,4-triazol-5-yl)-2-azaspiro[3.3]heptan-2-yl]methanone;
[6-[(5-cyclopropyl-2-methyl-pyrazol-3-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[[5-(difluoromethyl)thiazol-2-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethyl)-1H-pyrazol-3-yl]methyl]-2,6-diazaspiro[3.3]heptan-2-yl]methanone;
[6-[3-(1-hydroxycyclopropyl)-1H-1,2,4-triazol-5-yl]-2-azaspiro[3.3]heptan-2-yl]-[6-([1,2,4]triazolo[1,5-a]pyridin-6-ylmethyl)-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-(3-triflylbenzyl)-2-azaspiro[3.3]heptan-2-yl]methanone;
[6-(3-cyclopropyl-1H-1,2,4-triazol-5-yl)-2-azaspiro[3.3]heptan-2-yl]-[6-[[1-(difluoromethyl)pyrazol-4-yl]methyl]-2,6-diazaspiro[3.3]heptan-2-yl]methanone;
4-[[2-[6-[3-(1-hydroxycyclopropyl)-1H-1,2,4-triazol-5-yl]-2-azaspiro[3.3]heptane-2-carbonyl]-2-azaspiro[3.3]heptan-6-yl]methyl]-2-(trifluoromethyl)benzonitrile;
[6-(5-cyclopropyl-4H-1,2,4-triazol-3-yl)-2-azaspiro[3.3]heptan-2-yl]-[6-[2-(trifluoromethyl)pyrimidin-5-yl]-2-azaspiro[3.3]heptan-2-yl]methanone;
[6-(3-cyclopropyl-1H-1,2,4-triazol-5-yl)-2-azaspiro[3.3]heptan-2-yl]-[6-[1-(2,2,2-trifluoroethyl)pyrazol-4-yl]oxy-2-azaspiro[3.3]heptan-2-yl]methanone;
[6-(3-cyclopropyl-1H-1,2,4-triazol-5-yl)-2-azaspiro[3.3]heptan-2-yl]-[3-[2-fluoro-4-(trifluoromethdylsulfonimidoyl)benzyl]oxyazetidin-1-yl]methanone;
[6-[2-mesyl-4-(trifluoromethyl)benzyl]-2-azaspiro[3.3]heptan-2-yl]-[6-(1H-1,2,4-triazol-5-yl)-2-azaspiro[3.3]heptan-2-yl]methanone;
[6-[3-(oxetan-3-yl)-1H-1,2,4-triazol-5-yl]-2-azaspiro[3.3]heptan-2-yl]-[6-[[4-(trifluoromethyl)pyrazol-1-yl]methyl]-2-azaspiro[3.3]heptan-2-yl]methanone;
[6-(1H-1,2,4-triazol-5-yl)-2-azaspiro[3.3]heptan-2-yl]-[3-[4-[1-(trifluoromethyl)cyclopropyl]phenyl]azetidin-1-yl]methanone;
[6-[3-(1-aminocyclopropyl)-1H-1,2,4-triazol-5-yl]-2-azaspiro[3.3]heptan-2-yl]-[6-[4-fluoro-2-(trifluoromethyl)benzyl]-2-azaspiro[3.3]heptan-2-yl]methanone;
[6-[(3-cyclopropyl-1,2,4-thiadiazol-5-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[3-[4-(trifluoromethylsulfonimidoyl)phenyl]azetidin-1-yl]methanone; methanone;
[6-[3-(1-hydroxycyclopropyl)-1H-1,2,4-triazol-5-yl]-2-azaspiro[3.3]heptan-2-yl]-[6-[3-(trifluoromethylsulfonimidoyl)benzyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-methyl-1-(2,2,2-trifluoroethyl)pyrazol-4-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethyl)-1H-imidazol-2-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[2-(trifluoromethyl)oxazol-5-yl]methyl]-2,6-diazaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[4-(trifluoromethyl)thiazol-2-yl]methyl]-2,6-diazaspiro[3.3]heptan-2-yl]methanone;
[6-[(3-cyclopropyl-1H-pyrazol-5-yl)methyl]-2-azaspiro[3.3]heptan-2-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-([1,2,4]triazolo[1,5-a]pyridin-6-ylmethyl)-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[3-[6-[3-hydroxy-3-(trifluoromethyl)azetidin-1-yl]-3-pyridyl]azetidin-1-yl]methanone;
[6-[3-(1-fluorocyclopropyl)-1H-1,2,4-triazol-5-yl]-2-azaspiro[3.3]heptan-2-yl]-[6-[[5-(trifluoromethyl)pyrimidin-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-(3-hydroxy-1,2,4-triazol-1-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[3-fluoro-5-(trifluoromethylsulfonimidoyl)benzyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[2-methyl-5-(trifluoromethyl)pyrazol-3-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[2-(difluoromethyl)thiazol-5-yl]methyl]-2,6-diazaspiro[3.3]heptan-2-yl]methanone;
[6-[3-(1-fluorocyclopropyl)-1H-1,2,4-triazol-5-yl]-2-azaspiro[3.3]heptan-2-yl]-[6-[(5-triflyl-2-pyridyl)methyl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[(6R)-6-(4-triflylbenzyl)-2-azaspiro[3.4]octan-2-yl]methanone;
[6-(3-cyclopropyl-1H-1,2,4-triazol-5-yl)-2-azaspiro[3.3]heptan-2-yl]-[6-(3-fluoro-5-triflyl-benzyl)-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[4-(trifluoromethyl)isothiazol-5-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[2-(trifluoromethyl)pyrazol-3-yl]methyl]-2,6-diazaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[3-(trifluoromethylsulfonimidoyl)phenyl]methyl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[2-methyl-4-(trifluoromethyl)pyrazol-3-yl]methyl]-2,6-diazaspiro[3.3]heptan-2-yl]methanone;
[6-[(4-tert-butyl-1H-imidazol-2-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[3-(methylsulfonimidoyl)-5-(trifluoromethyl)benzyl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[5-(trifluoromethyl)-2-pyridyl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)-1,2,4-oxadiazol-5-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)isoxazol-4-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[2-methyl-4-(trifluoromethyl)pyrazol-3-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[3-fluoro-5-(trifluoromethyl)-2-pyridyl]oxy]-2-azaspiro[3.3]heptan-2-yl]methanone;
[6-(3-cyclopropyl-1H-1,2,4-triazol-5-yl)-2-azaspiro[3.3]heptan-2-yl]-[3-[2-[(3R)-3-(trifluoromethyl)pyrrolidino]pyrimidin-5-yl]azetidin-1-yl]methanone;
[6-(3-cyclopropyl-1H-1,2,4-triazol-5-yl)-2-azaspiro[3.3]heptan-2-yl]-[(6R)-6-(4-triflylbenzyl)-2-azaspiro[3.4]octan-2-yl]methanone;
[6-[3-(3,3-difluorocyclobutyl)-1H-1,2,4-triazol-5-yl]-2-azaspiro[3.3]heptan-2-yl]-[6-[[3-(trifluoromethylsulfonimidoyl)phenyl]methyl]-2-azaspiro[3.3]heptan-2-yl]methanone;
5-[[(6R)-2-[6-(5-cyclopropyl-4H-1,2,4-triazol-3-yl)-2-azaspiro[3.3]heptane-2-carbonyl]-2-azaspiro[3.4]octan-6-yl]methyl]-2-(trifluoromethyl)isonicotinonitrile;
[6-[3-(1-hydroxycyclopropyl)-1H-1,2,4-triazol-5-yl]-2-azaspiro[3.3]heptan-2-yl]-[6-[3-mesyl-5-(trifluoromethyl)benzyl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[[4-(trifluoromethyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)isoxazol-5-yl]methyl]-2,6-diazaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[[3-(trifluoromethyl)pyrazol-1-yl]methyl]-2-azaspiro[3.3]heptan-2-yl]methanone;
[3-[3-(4-fluorophenyl)-1-bicyclo[1.1.1]pentanyl]azetidin-1-yl]-[6-[3-(1-hydroxycyclopropyl)-1H-1,2,4-triazol-5-yl]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethyl)isothiazol-3-yl]methyl]-2,6-diazaspiro[3.3]heptan-2-yl]methanone;
[6-[(5-chloro-3-fluoro-2-pyridyl)methyl]-2-azaspiro[3.3]heptan-2-yl]-[6-[3-(1-hydroxycyclopropyl)-1H-1,2,4-triazol-5-yl]-2-azaspiro[3.3]heptan-2-yl]methanone;
4-[[2-[6-[3-(3,3-difluorocyclobutyl)-1H-1,2,4-triazol-5-yl]-2-azaspiro[3.3]heptane-2-carbonyl]-2-azaspiro[3.3]heptan-6-yl]methyl]-2-(trifluoromethyl)benzonitrile;
[6-(5-cyclopropyl-4H-1,2,4-triazol-3-yl)-2-azaspiro[3.3]heptan-2-yl]-[6-[3-fluoro-5-(trifluoromethyl)-2-pyridyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[2-(2,2,2-trifluoroethyl)-5-(trifluoromethyl)pyrazol-3-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-(3-dimethylphosphoryl-5-fluoro-benzyl)-2-azaspiro[3.3]heptan-2-yl]methanone;
[6-(3-cyclopropyl-1H-1,2,4-triazol-5-yl)-2-azaspiro[3.3]heptan-2-yl]-[6-[[1-methyl-5-(trifluoromethyl)pyrazol-3-yl]methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[5-(trifluoromethyl)-2-pyridyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)-1,2,4-oxadiazol-5-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-(1H-pyrazolo[4,3-b]pyridin-5-ylmethyl)-2-azaspiro[3.3]heptan-2-yl]methanone;
[6-(3-cyclopropyl-1H-1,2,4-triazol-5-yl)-2-azaspiro[3.3]heptan-2-yl]-[3-[3-(4-fluoro-2-mesyl-phenyl)-1-bicyclo[1.1.1]pentanyl]azetidin-1-yl]methanone;
[6-[(3-cyclopropyl-5-methyl-isoxazol-4-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[4-(trifluoromethylsulfonimidoyl)phenyl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-methyl-5-(trifluoromethyl)pyrazol-4-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[2-(2,2,2-trifluoroethyl)triazol-4-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[4-(2,2,2-trifluoroethyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[2-methyl-4-(trifluoromethyl)pyrazol-3-yl]methyl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[6-(1H-pyrazolo[4,3-b]pyridin-5-ylmethyl)-2-azaspiro[3.3]heptan-2-yl]methanone;
[6-(3-cyclopropyl-1H-1,2,4-triazol-5-yl)-2-azaspiro[3.3]heptan-2-yl]-[6-[[4-(trifluoromethyl)oxazol-2-yl]methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[5-(trifluoromethyl)pyrimidin-2-yl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[3-(2,2,2-trifluoroethyl)-1,2,4-oxadiazol-5-yl]methyl]-2,6-diazaspiro[3.3]heptan-2-yl]methanone;
5-[[(6S)-2-[6-[3-(1-hydroxy-cyclopropyl)-1H-1,2,4-triazol-5-yl]-2-azaspiro[3.3]heptane-2-carbonyl]-2-azaspiro[3.4]octan-6-yl]oxy]-2-(trifluoromethyl)isonicotinonitrile;
[6-[3-(1-hydroxycyclopropyl)-1H-1,2,4-triazol-5-yl]-2-azaspiro[3.3]heptan-2-yl]-[3-[4-(trifluoromethylsulfonimidoyl)phenyl]azetidin-1-yl]methanone;
[6-(3-cyclopropyl-1H-1,2,4-triazol-5-yl)-2-azaspiro[3.3]heptan-2-yl]-[6-[[1-(2,2,2-trifluoroethyl)imidazol-2-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[3-[3-[[5-(trifluoromethyl)pyrazin-2-yl]-amino]-1-bicyclo[1.1.1]-pentanyl]azetidin-1-yl]methanone;
[6-(3-cyclopropyl-1H-1,2,4-triazol-5-yl)-2-azaspiro[3.3]heptan-2-yl]-[6-[[4-(trifluoromethyl)isothiazol-5-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethyl)oxazol-4-yl]methyl]-2,6-diazaspiro[3.3]heptan-2-yl]methanone;
[3-[3-(3-cyclopropyl-1H-pyrazol-5-yl)-1-bicyclo[1.1.1]pentanyl]azetidin-1-yl]-[6-[3-(3,3-difluorocyclobutyl)-1H-1,2,4-triazol-5-yl]-2-azaspiro[3.3]heptan-2-yl]methanone;
[6-(3-cyclopropyl-1H-1,2,4-triazol-5-yl)-2-azaspiro[3.3]heptan-2-yl]-[7-(4-triflylbenzyl)-2,7-diazaspiro[3.4]octan-2-yl]methanone;
[6-(3-cyclopropyl-1H-1,2,4-triazol-5-yl)-2-azaspiro[3.3]heptan-2-yl]-[6-[[4-(trifluoromethyl)thiazol-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[3-[3-(3-fluoro-5-methoxy-phenyl)-1-bicyclo[1.1.1]pentanyl]azetidin-1-yl]methanone;
[6-[3-(1-hydroxycyclopropyl)-1H-1,2,4-triazol-5-yl]-2-azaspiro-[3.3]heptan-2-yl]-[4-[3-[5-[1-(trifluoromethyl)cyclopropyl]-1,3,4-oxadiazol-2-yl]-1-bicyclo-[1.1.1]pentanyl]piperidino]methanone;
[6-(3-cyclopropyl-1H-1,2,4-triazol-5-yl)-2-azaspiro[3.3]heptan-2-yl]-[3-[3-[5-(trifluoromethyl)pyrazin-2-yl]oxy-1-bicyclo[1.1.1]pentanyl]azetidin-1-yl]methanone;
[6-(3-cyclopropyl-1H-1,2,4-triazol-5-yl)-2-azaspiro[3.3]heptan-2-yl]-[3-[2-[(3 S)-3-(trifluoromethyl)pyrrolidino]pyrimidin-5-yl]azetidin-1-yl]methanone;
[6-(3-cyclopropyl-1H-1,2,4-triazol-5-yl)-2-azaspiro[3.3]heptan-2-yl]-[6-[[1-(trifluoromethyl)imidazol-2-yl]methyl]-2,6-diazaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[[6-(trifluoromethoxy)-3-pyridyl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[4-[1-(trifluoromethyl)cyclopropyl]-pyrazol-1-yl]methyl]-2-azaspiro[3.3]heptan-2-yl]methanone;
[3-[3-(3,5-dimethylpyrazol-1-yl)-1-bicyclo[1.1.1]pentanyl]azetidin-1-yl]-[6-[3-(1-hydroxycyclopropyl)-1H-1,2,4-triazol-5-yl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)isothiazol-5-yl]methyl]-2,6-diazaspiro[3.3]heptan-2-yl]methanone;
[6-[3-(1-hydroxycyclopropyl)-1H-1,2,4-triazol-5-yl]-2-azaspiro[3.3]heptan-2-yl]-[7-(4-triflylbenzyl)-2,7-diazaspiro[3.4]octan-2-yl]methanone;
[6-[2-fluoro-4-(trifluoromethylsulfonimidoyl)benzyl]-2-azaspiro[3.3]heptan-2-yl]-[6-[3-(1-hydroxycyclopropyl)-1H-1,2,4-triazol-5-yl]-2-azaspiro[3.3]heptan-2-yl]methanone;
[6-[3-(oxetan-3-yl)-1H-1,2,4-triazol-5-yl]-2-azaspiro[3.3]heptan-2-yl]-[6-[[5-(trifluoromethyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[2-(2,2,2-trifluoroethyl)pyrazol-3-yl]methyl]-2,6-diazaspiro[3.3]heptan-2-yl]methanone;
3-[[2-[6-(3-cyclopropyl-1H-1,2,4-triazol-5-yl)-2-azaspiro[3.3]heptane-2-carbonyl]-2,6-diazaspiro[3.3]heptan-6-yl]methyl]-2-pyridone;
1-[[2-[6-[3-(1-hydroxycyclopropyl)-1H-1,2,4-triazol-5-yl]-2-azaspiro[3.3]heptane-2-carbonyl]-2-azaspiro[3.3]heptan-6-yl]methyl]pyrazole-4-carbonitrile;
[6-(3-cyclopropyl-1H-1,2,4-triazol-5-yl)-2-azaspiro[3.3]heptan-2-yl]-[6-[[2-methyl-5-(trifluoromethyl)triazol-4-yl]methyl]-2-azaspiro[3.3]heptan-2-yl]methanone;
[6-(1H-1,2,4-triazol-5-yl)-2-azaspiro[3.3]heptan-2-yl]-[6-[[5-(trifluoromethoxy)-2-pyridyl]methyl]-2-azaspiro[3.3]heptan-2-yl]methanone;
[6-(3-cyclopropyl-1H-1,2,4-triazol-5-yl)-2-azaspiro[3.3]heptan-2-yl]-[3-[3-[4-(trifluoromethyl)phenyl]sulfonyl-1-bicyclo[1.1.1]pentanyl]azetidin-1-yl]methanone;
[6-(3-cyclopropyl-1H-1,2,4-triazol-5-yl)-2-azaspiro[3.3]heptan-2-yl]-[7-[4-(trifluoromethyl)phenyl]sulfonyl-2-azaspiro[3.5]nonan-2-yl]methanone;
1-[[2-[6-(3-cyclopropyl-1H-1,2,4-triazol-5-yl)-2-azaspiro[3.3]heptane-2-carbonyl]-2-azaspiro[3.3]heptan-6-yl]methyl]-5-(difluoromethoxy)-2-pyridone;
[6-(3-cyclopropyl-1H-1,2,4-triazol-5-yl)-2-azaspiro[3.3]heptan-2-yl]-[3-[3-(3,5-dimethylpyrazol-1-yl)-1-bicyclo[1.1.1]pentanyl]azetidin-1-yl]methanone;
[6-[3-(1-hydroxycyclopropyl)-1H-1,2,4-triazol-5-yl]-2-azaspiro[3.3]heptan-2-yl]-[(6S)-6-(3-triflylbenzyl)-2-azaspiro[3.4]octan-2-yl]methanone;
[6-[3-(1-hydroxycyclopropyl)-1H-1,2,4-triazol-5-yl]-2-azaspiro[3.3]heptan-2-yl]-[6-[[1-(trifluoromethyl)pyrazol-4-yl]methyl]-2-azaspiro[3.3]heptan-2-yl]methanone;
[6-[3-(oxetan-3-yl)-1H-1,2,4-triazol-5-yl]-2-azaspiro[3.3]heptan-2-yl]-[6-(3-triflylbenzyl)-2-azaspiro[3.3]heptan-2-yl]methanone;
[6-(3-cyclopropyl-1H-1,2,4-triazol-5-yl)-2-azaspiro[3.3]heptan-2-yl]-[6-[[1-methyl-5-(trifluoromethyl)pyrazol-4-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[4-(trifluoromethylsulfonimidoyl)phenoxy]-2-azaspiro[3.3]heptan-2-yl]methanone;
[6-(3-cyclopropyl-1H-1,2,4-triazol-5-yl)-2-azaspiro[3.3]heptan-2-yl]-[6-[(5-triflyl-3-pyridyl)methyl]-2-azaspiro[3.3]heptan-2-yl]methanone;
[6-(3-cyclopropyl-1H-1,2,4-triazol-5-yl)-2-azaspiro[3.3]heptan-2-yl]-[7-[[4-(trifluoromethyl)thiazol-2-yl]methyl]-2,7-diazaspiro[3.5]nonan-2-yl]methanone;
[6-[[3-(cyclopropylmethyl)-1,2,4-oxadiazol-5-yl]methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-(1H-1,2,4-triazol-5-yl)-2-azaspiro[3.3]heptan-2-yl]-[6-[3-(trifluoromethylsulfonimidoyl)benzyl]-2-azaspiro[3.3]heptan-2-yl]methanone;
[6-(3-cyclopropyl-1H-1,2,4-triazol-5-yl)-2-azaspiro[3.3]heptan-2-yl]-[6-[4-fluoro-2-(methylsulfonimidoyl)benzyl]-2-azaspiro[3.3]heptan-2-yl]methanone;
[6-(3-cyclopropyl-1H-1,2,4-triazol-5-yl)-2-azaspiro[3.3]heptan-2-yl]-[3-[3-[[5-(trifluoromethyl)-2-pyridyl]oxy]-1-bicyclo[1.1.1]pentanyl]azetidin-1-yl]methanone;
[6-[(5-chloro-3-fluoro-2-pyridyl)methyl]-2-azaspiro[3.3]heptan-2-yl]-[6-[3-(3,3-difluorocyclobutyl)-1H-1,2,4-triazol-5-yl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[[5-[1-(trifluoromethyl)cyclopropyl]-1,3,4-oxadiazol-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethylsulfonimidoyl)-2-pyridyl]methyl]-2-azaspiro[3.3]heptan-2-yl]methanone;
[6-[(4-tert-butylthiazol-2-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[(6R)-6-(3-triflylbenzyl)-2-azaspiro[3.4]octan-2-yl]methanone;
[6-[3-fluoro-5-(trifluoromethyl)benzyl]-2-azaspiro[3.3]heptan-2-yl]-[6-[3-(oxetan-3-yl)-1H-1,2,4-triazol-5-yl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(1,1-difluoroethyl)-1,2,4-oxadiazol-5-yl]methyl]-2,6-diazaspiro[3.3]heptan-2-yl]methanone;
[6-[[1-methyl-5-(trifluoromethyl)pyrazol-3-yl]methyl]-2-azaspiro[3.3]heptan-2-yl]-[6-[3-(oxetan-3-yl)-1H-1,2,4-triazol-5-yl]-2-azaspiro[3.3]heptan-2-yl]methanone;
[3-[4-(cyclopropylsulfonimidoyl)phenyl]azetidin-1-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-(3-cyclopropyl-1,2,4-triazol-1-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[[4-(trifluoromethyl)-1H-pyrazol-3-yl]methyl]-2-azaspiro[3.3]heptan-2-yl]methanone;
[6-[3-(oxetan-3-yl)-1H-1,2,4-triazol-5-yl]-2-azaspiro[3.3]heptan-2-yl]-[6-[[5-(trifluoromethyl)-3-pyridyl]methyl]-2-azaspiro[3.3]heptan-2-yl]methanone;
3-[[2-[6-[3-(1-hydroxycyclopropyl)-1H-1,2,4-triazol-5-yl]-2-azaspiro[3.3]heptane-2-carbonyl]-2-azaspiro[3.3]heptan-6-yl]methyl]-5-(trifluoromethyl)benzonitrile;
[6-[3-(3,3-difluorocyclobutyl)-1H-1,2,4-triazol-5-yl]-2-azaspiro-[3.3]heptan-2-yl]-[6-[[5-(trifluoromethyl)pyrazin-2-yl]methyl]-2,6-diazaspiro[3.3]heptan-2-yl]methanone;
[3-[4-(3,5-diisopropylpyrazol-1-yl)phenyl]azetidin-1-yl]-[6-[3-(1-hydroxycyclopropyl)-1H-1,2,4-triazol-5-yl]-2-azaspiro[3.3]heptan-2-yl]methanone;
3-fluoro-4-[[2-[6-[3-(1-hydroxycyclopropyl)-1H-1,2,4-triazol-5-yl]-2-azaspiro[3.3]heptane-2-carbonyl]-2-azaspiro[3.3]heptan-6-yl]methyl]benzonitrile;
[6-[3-mesyl-4-(trifluoromethyl)benzyl]-2-azaspiro[3.3]heptan-2-yl]-[6-(1H-1,2,4-triazol-5-yl)-2-azaspiro[3.3]heptan-2-yl]methanone;
[6-[4-fluoro-2-(trifluoromethyl)benzyl]-2-azaspiro[3.3]heptan-2-yl]-[6-[3-(oxetan-3-yl)-1H-1,2,4-triazol-5-yl]-2-azaspiro[3.3]heptan-2-yl]methanone;
[6-[3-(oxetan-3-yl)-1H-1,2,4-triazol-5-yl]-2-azaspiro[3.3]heptan-2-yl]-[6-(4-triflylbenzyl)-2-azaspiro[3.3]heptan-2-yl]methanone;
[7-[(5-chloro-2-pyridyl)methyl]-2-azaspiro[3.5]nonan-2-yl]-[6-[3-(3,3-difluorocyclobutyl)-1H-1,2,4-triazol-5-yl]-2-azaspiro[3.3]heptan-2-yl]methanone;
1-[[2-[6-(3-cyclopropyl-1H-1,2,4-triazol-5-yl)-2-azaspiro[3.3]heptane-2-carbonyl]-2-azaspiro[3.3]heptan-6-yl]methyl]-5-(trifluoromethoxy)-2-pyridone;
[6-[3-(3,3-difluorocyclobutyl)-1H-1,2,4-triazol-5-yl]-2-azaspiro[3.3]heptan-2-yl]-[6-[[3-(trifluoromethyl)isoxazol-5-yl]methyl]-2,6-diazaspiro[3.3]heptan-2-yl]methanone;
3-[[2-[6-[3-(oxetan-3-yl)-1H-1,2,4-triazol-5-yl]-2-azaspiro[3.3]heptane-2-carbonyl]-2-azaspiro[3.3]heptan-6-yl]methyl]-5-(trifluoromethyl)benzonitrile;
[6-(3-cyclopropyl-1H-1,2,4-triazol-5-yl)-2-azaspiro[3.3]heptan-2-yl]-[3-[3-(5-fluoro-3-pyridyl)-1-bicyclo[1.1.1]pentanyl]azetidin-1-yl]methanone;
[6-[3-(oxetan-3-yl)-1H-1,2,4-triazol-5-yl]-2-azaspiro[3.3]heptan-2-yl]-[6-[[5-(trifluoromethyl)pyrazin-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[(3-ethyl-1,2,4-thiadiazol-5-yl)methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[2-(trifluoromethyl)oxazol-4-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[1-(2,2,2-trifluoroethyl)pyrazol-4-yl]methyl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[[1-(2,2,2-trifluoroethyl)pyrazol-4-yl]methyl]-2-azaspiro[3.3]heptan-2-yl]methanone;
1-[[2-[6-(3-cyclopropyl-1H-1,2,4-triazol-5-yl)-2-azaspiro[3.3]heptane-2-carbonyl]-2-azaspiro[3.3]heptan-6-yl]methyl]-4-(trifluoromethyl)pyridin-2-one;
5-chloro-2-[3-[1-[6-(5-cyclopropyl-4H-1,2,4-triazol-3-yl)-2-azaspiro[3.3]heptane-2-carbonyl]azetidin-3-yl]-1-bicyclo[1.1.1]pentanyl]-N-ethyl-benzamide;
[6-[2-fluoro-4-(trifluoromethyl)benzyl]-2-azaspiro[3.3]heptan-2-yl]-[6-[3-(oxetan-3-yl)-1H-1,2,4-triazol-5-yl]-2-azaspiro[3.3]heptan-2-yl]methanone;
[6-[3-(oxetan-3-yl)-1H-1,2,4-triazol-5-yl]-2-azaspiro[3.3]heptan-2-yl]-[6-[[6-(trifluoromethoxy)-3-pyridyl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-methyl-3-(trifluoromethyl)pyrazol-4-yl]methyl]-2-azaspiro[3.3]heptan-2-yl]methanone;
4-[[2-[6-[3-(oxetan-3-yl)-1H-1,2,4-triazol-5-yl]-2-azaspiro[3.3]heptane-2-carbonyl]-2-azaspiro[3.3]heptan-6-yl]methyl]-2-(trifluoromethyl)benzonitrile;
4-[[2-[6-(3-cyclopropyl-1H-1,2,4-triazol-5-yl)-2-azaspiro[3.3]heptane-2-carbonyl]-2-azaspiro[3.3]heptan-6-yl]methyl]-3-fluoro-benzonitrile;
[6-(3-cyclopropyl-1H-1,2,4-triazol-5-yl)-2-azaspiro[3.3]heptan-2-yl]-[3-[3-(4-fluorophenyl)-1-bicyclo[1.1.1]pentanyl]azetidin-1-yl]methanone;
[6-[[1-methyl-5-(trifluoromethyl)pyrazol-4-yl]methyl]-2-azaspiro[3.3]heptan-2-yl]-[6-[3-(oxetan-3-yl)-1H-1,2,4-triazol-5-yl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[[2-methyl-5-(trifluoromethyl)pyrazol-3-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[3-[4-(1-mesylcyclopropyl)phenyl]azetidin-1-yl]methanone;
[6-[3-(1-hydroxycyclopropyl)-1H-1,2,4-triazol-5-yl]-2-azaspiro[3.3]heptan-2-yl]-[6-[[2-methyl-4-(trifluoromethyl)pyrazol-3-yl]methyl]-2-azaspiro[3.3]heptan-2-yl]methanone;
4-[[2-[6-(5-cyclopropyl-4H-1,2,4-triazol-3-yl)-2-azaspiro[3.3]heptane-2-carbonyl]-2-azaspiro[3.3]heptan-6-yl]methyl]-6-(trifluoromethyl)picolinonitrile;
[6-(3-cyclopropyl-1H-1,2,4-triazol-5-yl)-2-azaspiro[3.3]heptan-2-yl]-[7-[[5-(trifluoromethyl)-1,3,4-oxadiazol-2-yl]methyl]-2-azaspiro[3.5]nonan-2-yl]methanone;
[6-(3-cyclopropyl-1H-1,2,4-triazol-5-yl)-2-azaspiro[3.3]heptan-2-yl]-[6-[[4-(trifluoromethyl)thiazol-2-yl]methyl]-2,6-diazaspiro[3.3]heptan-2-yl]methanone;
[6-[3-(1-hydroxycyclopropyl)-1H-1,2,4-triazol-5-yl]-2-azaspiro[3.3]heptan-2-yl]-[3-[4-(2-mesylphenyl)phenyl]azetidin-1-yl]methanone;
[6-[3-(oxetan-3-yl)-1H-1,2,4-triazol-5-yl]-2-azaspiro[3.3]heptan-2-yl]-[6-[[4-(trifluoromethylsulfonimidoyl)phenyl]methyl]-2-azaspiro[3.3]heptan-2-yl]methanone;
[6-[3-(oxetan-3-yl)-1H-1,2,4-triazol-5-yl]-2-azaspiro[3.3]heptan-2-yl]-[6-[[4-(trifluoromethyl)thiazol-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[2-(trifluoromethyl)thiazol-5-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[2-methyl-5-(trifluoromethyl)pyrazol-3-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[3-[3-(4-methylsulfonylphenyl)-1-bicyclo[1.1.1]pentanyl]azetidin-1-yl]methanone;
[6-(3-cyclopropyl-1H-1,2,4-triazol-5-yl)-2-azaspiro[3.3]heptan-2-yl]-[6-[[3-[1-(trifluoromethyl)cyclopropyl]-1,2,4-triazol-1-yl]methyl]-2-azaspiro[3.3]heptan-2-yl]methanone;
[6-[[5-(trifluoromethyl)pyrazin-2-yl]methyl]-2-azaspiro[3.3]heptan-2-yl]-[6-[3-(trifluoromethyl)-1H-1,2,4-triazol-5-yl]-2-azaspiro[3.3]heptan-2-yl]methanone;
[6-[(3-chloro-5-fluoro-2-pyridyl)methyl]-2-azaspiro[3.3]heptan-2-yl]-[6-(1H-1,2,4-triazol-5-yl)-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[3-[4-(1-mesylcyclopropyl)phenyl]azetidin-1-yl]methanone;
[6-(4-mesylbenzyl)-2-azaspiro[3.3]heptan-2-yl]-[6-[3-(trifluoromethyl)-1H-1,2,4-triazol-5-yl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[5-[1-(trifluoromethyl);
cyclopropyl]-1,3,4-oxadiazol-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-(2,2,2-trifluoroethyl)-5-(trifluoromethyl)pyrazol-3-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethyl)-1,2,4-oxadiazol-3-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethyl)-1H-imidazol-4-yl]methyl]-2,6-diazaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[[5-(trifluoromethyl)pyrazol-1-yl]methyl]-2-azaspiro[3.3]heptan-2-yl]methanone;
3-fluoro-4-[[2-[6-[3-(oxetan-3-yl)-1H-1,2,4-triazol-5-yl]-2-azaspiro[3.3]heptane-2-carbonyl]-2-azaspiro[3.3]heptan-6-yl]methyl]benzonitrile;
[6-(3-cyclopropyl-1H-1,2,4-triazol-5-yl)-2-azaspiro[3.3]heptan-2-yl]-[3-[3-[difluoro-[4-(trifluoromethyl)-phenyl]methyl]-1-bicyclo[1.1.1]-pentanyl]azetidin-1-yl]methanone;
[6-(3-cyclopropyl-1H-1,2,4-triazol-5-yl)-2-azaspiro[3.3]heptan-2-yl]-[6-(1H-pyrazol-3-ylmethyl)-2,6-diazaspiro[3.3]heptan-2-yl]methanone;
[3-[3-(5-neopentyl-1,3,4-thiadiazol-2-yl)-1-bicyclo[1.1.1]pentanyl]azetidin-1-yl]-[6-[3-(oxetan-3-yl)-1H-1,2,4-triazol-5-yl]-2-azaspiro[3.3]heptan-2-yl]methanone;
1-[[2-[6-(3-cyclopropyl-1H-1,2,4-triazol-5-yl)-2-azaspiro[3.3]heptane-2-carbonyl]-2-azaspiro[3.3]heptan-6-yl]methyl]pyrazole-4-carbonitrile;
[6-(5-cyclopropyl-4H-1,2,4-triazol-3-yl)-2-azaspiro[3.3]heptan-2-yl]-[3-[4-(2,5-dimethylthiazol-4-yl)phenyl]azetidin-1-yl]methanone;
[6-(5-cyclopropyl-4H-1,2,4-triazol-3-yl)-2-azaspiro[3.3]heptan-2-yl]-[6-[2-mesyl-4-(trifluoromethyl)phenyl]-2-azaspiro[3.3]heptan-2-yl]methanone;
[6-(3-cyclopropyl-1H-1,2,4-triazol-5-yl)-2-azaspiro[3.3]heptan-2-yl]-[7-[[6-(trifluoromethyl)-3-pyridyl]methyl]-2,7-diazaspiro[3.4]octan-2-yl]methanone;
[6-(3-cyclopropyl-1H-1,2,4-triazol-5-yl)-2-azaspiro[3.3]heptan-2-yl]-[6-[6-(trifluoromethyl)-3-pyridyl]-2-azaspiro[3.3]heptan-2-yl]methanone;
[6-(5-cyclopropyl-4H-1,2,4-triazol-3-yl)-2-azaspiro[3.3]heptan-2-yl]-[3-[4-(4-methylimidazol-1-yl)phenyl]azetidin-1-yl]methanone;
[6-(3-cyclopropyl-1H-1,2,4-triazol-5-yl)-2-azaspiro[3.3]heptan-2-yl]-[3-[3-[[5-(trifluoromethyl)-2-pyridyl]-sulfonyl]-1-bicyclo[1.1.1]pentanyl]-azetidin-1-yl]methanone;
5-[[2-[6-[3-(oxetan-3-yl)-1H-1,2,4-triazol-5-yl]-2-azaspiro[3.3]heptane-2-carbonyl]-2-azaspiro[3.3]heptan-6-yl]methyl]-2-(trifluoromethyl)benzonitrile;
[6-(3-cyclopropyl-1H-1,2,4-triazol-5-yl)-2-azaspiro[3.3]heptan-2-yl]-[6-[3-mesyl-5-(trifluoromethyl)benzyl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[[1-methyl-3-(trifluoromethyl)pyrazol-4-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[4-[[5-(trifluoromethyl)pyrazin-2-yl]amino]cuban-1-yl]methanone;
[6-[3-(1-hydroxycyclopropyl)-1H-1,2,4-triazol-5-yl]-2-azaspiro[3.3]heptan-2-yl]-[3-[4-(3-neopentyltriazol-4-yl)phenyl]azetidin-1-yl]methanone;
[6-(3-cyclopropyl-1H-1,2,4-triazol-5-yl)-2-azaspiro[3.3]heptan-2-yl]-[6-[(5-triflyl-2-pyridyl)methyl]-2-azaspiro[3.3]heptan-2-yl]methanone;
[6-[3-(oxetan-3-yl)-1H-1,2,4-triazol-5-yl]-2-azaspiro[3.3]heptan-2-yl]-[7-[[5-(trifluoromethyl)-2-pyridyl]methyl]-2-azaspiro[3.5]nonan-2-yl]methanone;
[6-[3-(1-hydroxycyclopropyl)-1H-1,2,4-triazol-5-yl]-2-azaspiro[3.3]heptan-2-yl]-[6-[[1-methyl-5-(trifluoromethyl)pyrazol-4-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[5-[1-(trifluoromethyl)cyclopropyl]-1H-1,2,4-triazol-3-yl]methyl]-2-azaspiro[3.3]heptan-2-yl]methanone;
[6-(4-mesylbenzyl)-2-azaspiro[3.3]heptan-2-yl]-[6-[3-(oxetan-3-yl)-1H-1,2,4-triazol-5-yl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(difluoromethyl)-5-methyl-pyrazol-1-yl]methyl]-2-azaspiro[3.3]heptan-2-yl]methanone;
[6-[(3,5-difluoro-2-pyridyl)methyl]-2-azaspiro[3.3]heptan-2-yl]-[6-[3-(trifluoromethyl)-1H-1,2,4-triazol-5-yl]-2-azaspiro[3.3]heptan-2-yl]methanone;
[3-[4-(3-tert-butyl-5-methyl-pyrazol-1-yl)phenyl]azetidin-1-yl]-[6-[3-(1-hydroxycyclopropyl)-1H-1,2,4-triazol-5-yl]-2-azaspiro[3.3]heptan-2-yl]methanone;
[3-[4-(3-cyclopropyl-5-methyl-pyrazol-1-yl)phenyl]azetidin-1-yl]-[6-[3-(1-hydroxycyclopropyl)-1H-1,2,4-triazol-5-yl]-2-azaspiro[3.3]heptan-2-yl]methanone;
[6-[(3-cyclopropylpyrazol-1-yl)methyl]-2-azaspiro[3.3]heptan-2-yl]-[6-(3-cyclopropyl-1H-1,2,4-triazol-5-yl)-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[3-[4-[1-methyl-3-(trifluoromethyl)pyrazol-4-yl]phenyl]azetidin-1-yl]methanone;
[6-[3-(oxetan-3-yl)-1H-1,2,4-triazol-5-yl]-2-azaspiro[3.3]heptan-2-yl]-[6-[[5-(trifluoromethyl)-2-pyridyl]methyl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[3-[3-[[1-(trifluoromethyl)cyclopropyl]methylamino]-1-bicyclo[1.1.1]pentanyl]azetidin-1-yl]methanone;
[6-(5-cyclopropyl-4H-1,2,4-triazol-3-yl)-2-azaspiro[3.3]heptan-2-yl]-[6-[difluoro-[6-(trifluoromethyl)-3-pyridyl]methyl]-2-azaspiro[3.3]heptan-2-yl]methanone;
[6-[2-mesyl-4-(trifluoromethyl)benzyl]-2-azaspiro[3.3]heptan-2-yl]-[6-[3-(oxetan-3-yl)-1H-1,2,4-triazol-5-yl]-2-azaspiro[3.3]heptan-2-yl]methanone;
6-(5-cyclopropyl-4H-1,2,4-triazol-3-yl)-N-[[6-(trifluoromethyl)-3,4-diazatricyclo[7.1.1.03,7]undeca-4,6-dien-1-yl]methyl]-2-azaspiro[3.3]heptane-2-carboxamide;
[6-[3-(1-hydroxycyclopropyl)-1H-1,2,4-triazol-5-yl]-2-azaspiro[3.3]heptan-2-yl]-[6-[6-(trifluoromethyl)-3-pyridyl]-2-azaspiro[3.3]heptan-2-yl]methanone;
[6-[3-(oxetan-3-yl)-1H-1,2,4-triazol-5-yl]-2-azaspiro[3.3]heptan-2-yl]-[6-[4-(trifluoromethylsulfonimidoyl)benzyl]-2-azaspiro[3.3]heptan-2-yl]methanone;
[6-[(5-chloro-2-pyridyl)methyl]-2-azaspiro[3.3]heptan-2-yl]-[6-(1H-1,2,4-triazol-5-yl)-2-azaspiro[3.3]heptan-2-yl]methanone;
[3-[4-(4-cyclopropylimidazol-1-yl)phenyl]azetidin-1-yl]-[6-(5-cyclopropyl-4H-1,2,4-triazol-3-yl)-2-azaspiro[3.3]heptan-2-yl]methanone;
5-[[2-[6-(5-cyclopropyl-4H-1,2,4-triazol-3-yl)-2-azaspiro[3.3]heptane-2-carbonyl]-2-azaspiro[3.3]heptan-6-yl]methyl]-2-(trifluoromethyl)nicotinonitrile;
[6-(5-cyclopropyl-4H-1,2,4-triazol-3-yl)-2-azaspiro[3.3]heptan-2-yl]-[3-[4-[1-methyl-3-(trifluoromethyl)pyrazol-4-yl]phenyl]azetidin-1-yl]methanone;
[6-(2,4-difluorobenzyl)-2-azaspiro[3.3]heptan-2-yl]-[6-[3-(oxetan-3-yl)-1H-1,2,4-triazol-5-yl]-2-azaspiro[3.3]heptan-2-yl]methanone;
[6-(3-cyclopropyl-1H-1,2,4-triazol-5-yl)-2-azaspiro[3.3]heptan-2-yl]-[2-[[3-methyl-5-(trifluoromethyl)-2-pyridyl]oxy]-7-azaspiro[3.5]nonan-7-yl]methanone;
[6-[3-(3,3-difluorocyclobutyl)-1H-1,2,4-triazol-5-yl]-2-azaspiro[3.3]heptan-2-yl]-[6-[[4-(trifluoromethyl)triazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[(4-dimethylphosphoryl-2-methyl-pyrazol-3-yl)methyl]-2,6-diazaspiro[3.3]heptan-2-yl]methanone;
[3-[4-[5-cyclopropyl-3-(difluoromethyl)pyrazol-1-yl]phenyl]azetidin-1-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[3-[4-[4-(1-hydroxyethyl)-5-methyl-thiazol-2-yl]phenyl]azetidin-1-yl]methanone;
[6-(5-cyclopropyl-4H-1,2,4-triazol-3-yl)-2-azaspiro[3.3]heptan-2-yl]-[3-[4-(3,5-dimethyl-1,2,4-triazol-1-yl)phenyl]azetidin-1-yl]methanone; methanone;
[6-(5-cyclopropyl-4H-1,2,4-triazol-3-yl)-2-azaspiro[3.3]heptan-2-yl]-[4-[[6-(trifluoromethyl)-3-pyridyl]methyl]piperazino]methanone;
5-[[2-[6-(5-cyclopropyl-4H-1,2,4-triazol-3-yl)-2-azaspiro[3.3]heptane-2-carbonyl]-2-azaspiro[3.3]heptan-6-yl]methyl]-2-(trifluoromethyl)isonicotinonitrile;
[6-(3-cyclopropyl-1H-1,2,4-triazol-5-yl)-2-azaspiro[3.3]heptan-2-yl]-[6-[[3-(trifluoromethyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[fluoro-[6-(trifluoromethyl)pyridin-3-yl]methyl]-2-azaspiro[3.3]heptan-2-yl]methanone;
[6-[(5-cyclopropyl-3-methyl-pyrazol-1-yl)methyl]-2-azaspiro[3.3]heptan-2-yl]-[6-(3-cyclopropyl-1H-1,2,4-triazol-5-yl)-2-azaspiro[3.3]heptan-2-yl]methanone;
[6-[(3-cyclopropyl-5-methyl-pyrazol-1-yl)methyl]-2-azaspiro[3.3]heptan-2-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[3-(trifluoromethylsulfonimidoyl)phenoxy]-2-azaspiro[3.3]heptan-2-yl]methanone;
[6-(3-cyclopropyl-1H-1,2,4-triazol-5-yl)-2-azaspiro[3.3]heptan-2-yl]-[6-[2-fluoro-4-(trifluoromethylsulfonimidoyl)benzyl]-2-azaspiro[3.3]heptan-2-yl]methanone;
[6-[3-(trifluoromethyl)-1H-1,2,4-triazol-5-yl]-2-azaspiro[3.3]heptan-2-yl]-[6-(4-triflylbenzyl)-2,6-diazaspiro[3.3]heptan-2-yl]methanone;
[6-(5-cyclopropyl-4H-1,2,4-triazol-3-yl)-2-azaspiro[3.3]heptan-2-yl]-[3-[4-[3-(trifluoromethyl)-1H-pyrazol-5-yl]phenyl]azetidin-1-yl]methanone;
[6-[3-(1-aminocyclopropyl)-1H-1,2,4-triazol-5-yl]-2-azaspiro[3.3]heptan-2-yl]-[6-[2-fluoro-4-(trifluoromethyl)benzyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)-1H-pyrrolo[2,3-b]pyridin-6-yl]methyl]-2-azaspiro[3.3]heptan-2-yl]methanone;
5-[[2-[6-[3-(1-aminocyclopropyl)-1H-1,2,4-triazol-5-yl]-2-azaspiro[3.3]heptane-2-carbonyl]-2-azaspiro[3.3]heptan-6-yl]methyl]-2-(trifluoromethyl)benzonitrile;
3-[[2-[6-[3-(1-aminocyclopropyl)-1H-1,2,4-triazol-5-yl]-2-azaspiro[3.3]heptane-2-carbonyl]-2-azaspiro[3.3]heptan-6-yl]methyl]-5-(trifluoromethyl)benzonitrile;
[6-(3-cyclopropyl-1H-1,2,4-triazol-5-yl)-2-azaspiro[3.3]heptan-2-yl]-[6-[[1-methyl-3-(trifluoromethyl)pyrrolo[2,3-b]pyridin-6-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[4-(trifluoromethoxy)benzyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-(2-hydroxyethyl)-3-(trifluoromethyl)pyrrolo[2,3-b]pyridin-6-yl]methyl]-2-azaspiro[3.3]heptan-2-yl]methanone;
[6-(4-cyclopropylsulfonylbenzyl)-2-azaspiro[3.3]heptan-2-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[[4-[1-(trifluoromethyl)cyclopropyl]-imidazol-1-yl]methyl]-2-azaspiro[3.3]heptan-2-yl]methanone;
4-[[1-[6-(5-cyclopropyl-4H-1,2,4-triazol-3-yl)-2-azaspiro[3.3]heptane-2-carbonyl]-4-piperidyl]oxymethyl]-3-methoxy-benzonitrile;
[6-(5-cyclopropyl-4H-1,2,4-triazol-3-yl)-2-azaspiro[3.3]heptan-2-yl]-[6-[[5-[1-(trifluoromethyl)cyclopropyl]-1,3,4-oxadiazol-2-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[(5-dimethylphosphoryl-2-pyridyl)methyl]-2-azaspiro[3.3]heptan-2-yl]methanone;
[4-[5-chloro-1-[(2S)-3,3,3-trifluoro-2-hydroxy-propyl]indazol-3-yl]piperidino]-[6-(5-cyclopropyl-4H-1,2,4-triazol-3-yl)-2-azaspiro[3.3]heptan-2-yl]methanone;
[4-[5-chloro-1-[(2R)-3,3,3-trifluoro-2-hydroxy-propyl]indazol-3-yl]piperidino]-[6-(5-cyclopropyl-4H-1,2,4-triazol-3-yl)-2-azaspiro[3.3]heptan-2-yl]methanone;
[6-(3-cyclopropyl-1H-1,2,4-triazol-5-yl)-2-azaspiro[3.3]heptan-2-yl]-[3-[3-[[6-(trifluoromethyl)-3-pyridyl]methyl]-1-bicyclo[1.1.1]pentanyl]azetidin-1-yl]methanone;
3-[[2-[6-(3-cyclopropyl-1H-1,2,4-triazol-5-yl)-2-azaspiro[3.3]heptane-2-carbonyl]-2-azaspiro[3.3]heptan-6-yl]methyl]-5-(trifluoromethyl)benzonitrile;
[7-(4-chloro-2-mesyl-phenyl)-2,7-diazaspiro[3.5]nonan-2-yl]-[6-(5-cyclopropyl-4H-1,2,4-triazol-3-yl)-2-azaspiro[3.3]heptan-2-yl]methanone;
[4-[6-(5-cyclopropyl-4H-1,2,4-triazol-3-yl)-2-azaspiro[3.3]heptane-2-carbonyl]piperazino]-(4-ethylphenyl)methanone;
[4-[6-(5-cyclopropyl-4H-1,2,4-triazol-3-yl)-2-azaspiro[3.3]heptane-2-carbonyl]piperazino]-[5-(2-pyridyl)-2-thienyl]methanone;
[4-[6-(5-cyclopropyl-4H-1,2,4-triazol-3-yl)-2-azaspiro[3.3]heptane-2-carbonyl]piperazino]-[4-(methylamino)phenyl]methanone;
[4-[6-(5-cyclopropyl-4H-1,2,4-triazol-3-yl)-2-azaspiro[3.3]heptane-2-carbonyl]piperazino]-(4-methyl-2-thienyl)methanone;
[6-(5-cyclopropyl-4H-1,2,4-triazol-3-yl)-2-azaspiro[3.3]heptan-2-yl]-[3-[4-[3-(trifluoromethyl)pyrazol-1-yl]phenyl]azetidin-1-yl]methanone;
[6-(5-cyclopropyl-4H-1,2,4-triazol-3-yl)-2-azaspiro[3.3]heptan-2-yl]-[6-[[4-methyl-3-(trifluoromethyl)-1H-pyrazol-5-yl]methyl]-2,6-diazaspiro[3.3]heptan-2-yl]methanone;
[3-[4-[3-cyclopropyl-5-(difluoromethyl)pyrazol-1-yl]phenyl]azetidin-1-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[4-[[5-(trifluoromethyl)-2-pyridyl]methyl]piperazino]methanone;
[6-(5-cyclopropyl-4H-1,2,4-triazol-3-yl)-2-azaspiro[3.3]heptan-2-yl]-[6-[(2,4-dimethylthiazol-5-yl)methyl]-2,6-diazaspiro[3.3]heptan-2-yl]methanone;
[6-[(5-chloro-1,3-dimethyl-pyrazol-4-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[4-[(2-methyloxazol-4-yl)methyl]piperazino]methanone;
[6-(3-cyclopropyl-1H-1,2,4-triazol-5-yl)-2-azaspiro[3.3]heptan-2-yl]-[4-[(4-methylthiazol-5-yl)methyl]piperazino]methanone;
[4-[(5-chloro-1,3-dimethyl-pyrazol-4-yl)methyl]piperazino]-[6-(3-cyclopropyl-1H-1,2,4-triazol-5-yl)-2-azaspiro[3.3]heptan-2-yl]methanone;
4-[[2-[6-(3-cyclopropyl-1H-1,2,4-triazol-5-yl)-2-azaspiro[3.3]heptane-2-carbonyl]-2,6-diazaspiro[3.3]heptan-6-yl]methyl]-3-(trifluoromethyl)benzonitrile;
[6-(3-cyclopropyl-1H-1,2,4-triazol-5-yl)-2-azaspiro[3.3]heptan-2-yl]-[6-[[5-(difluoromethyl)-3-methyl-pyrazol-1-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[3-dimethylphosphoryl-5-(trifluoromethyl)benzyl]-2-azaspiro[3.3]heptan-2-yl]methanone;
4-[[1-[6-(5-cyclopropyl-4H-1,2,4-triazol-3-yl)-2-azaspiro[3.3]heptane-2-carbonyl]-4-piperidyl]oxymethyl]-3-methoxy-benzamide;
[6-[(3-tert-butyl-1,2,4-oxadiazol-5-yl)methyl]-2,6-diazaspiro[3.3]heptan-2-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[6-(pyrazolo[1,5-a]pyridin-3-ylmethyl)-2,6-diazaspiro[3.3]heptan-2-yl]methanone;
[6-(5-cyclopropyl-4H-1,2,4-triazol-3-yl)-2-azaspiro[3.3]heptan-2-yl]-[6-[(3-phenyl-1,2,4-oxadiazol-5-yl)methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[2-mesyl-4-(trifluoromethyl)benzyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[4-dimethylphosphoryl-2-(trifluoromethyl)benzyl]-2-azaspiro[3.3]heptan-2-yl]methanone;
[3-[(4-chloro-2-methoxy-benzyl)-methyl-amino]azetidin-1-yl]-[6-(5-cyclopropyl-4H-1,2,4-triazol-3-yl)-2-azaspiro[3.3]heptan-2-yl]methanone;
[2-(4-chloro-2-mesyl-benzyl)-6-azaspiro[3.4]octan-6-yl]-[6-(5-cyclopropyl-4H-1,2,4-triazol-3-yl)-2-azaspiro[3.3]heptan-2-yl]methanone;
[4-[(4-chloro-2-methoxy-benzyl)-methyl-amino]piperidino]-[6-(5-cyclopropyl-4H-1,2,4-triazol-3-yl)-2-azaspiro[3.3]heptan-2-yl]methanone;
[3-[[(4-chloro-2-methoxy-benzyl)-methyl-amino]methyl]azetidin-1-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[6-[(4-methylthiazol-2-yl)methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[(2-methylthiazol-4-yl)methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[(1,5-dimethylpyrazol-4-yl)methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[6-[(2,4-dimethyloxazol-5-yl)methyl]-2,6-diazaspiro[3.3]heptan-2-yl]methanone;
5-chloro-2-[[1-[6-(3-cyclopropyl-1H-1,2,4-triazol-5-yl)-2-azaspiro[3.3]heptane-2-carbonyl]-4-piperidyl]methoxy]nicotinamide;
[6-[(3-cyclopropyl-1H-pyrazol-5-yl)methyl]-2,6-diazaspiro[3.3]heptan-2-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[(2-methylpyrazol-3-yl)methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[(1-methylpyrazol-3-yl)methyl]-2,6-diazaspiro[3.3]heptan-2-yl]methanone;
[6-(5-cyclopropyl-4H-1,2,4-triazol-3-yl)-2-azaspiro[3.3]heptan-2-yl]-[4-[[6-(trifluoromethyl)-3-pyridyl]oxymethyl]piperidino]methanone;
4-[[1-[6-(3-cyclopropyl-1H-1,2,4-triazol-5-yl)-2-azaspiro[3.3]heptane-2-carbonyl]-4-piperidyl]oxymethyl]-2-methoxy-benzonitrile;
[6-(3-cyclopropyl-1H-1,2,4-triazol-5-yl)-2-azaspiro[3.3]heptan-2-yl]-[4-[2-fluoro-4-(trifluoromethoxy)benzyl]oxypiperidino]methanone;
[6-(3-cyclopropyl-1H-1,2,4-triazol-5-yl)-2-azaspiro[3.3]heptan-2-yl]-[4-[2-fluoro-4-(trifluoromethyl)benzyl]oxypiperidino]methanone;
[6-(3-cyclopropyl-1H-1,2,4-triazol-5-yl)-2-azaspiro[3.3]heptan-2-yl]-[4-[2-mesyl-4-(trifluoromethyl)benzyl]-oxypiperidino]methanone;
5-chloro-2-[[1-[6-(3-cyclopropyl-1H-1,2,4-triazol-5-yl)-2-azaspiro[3.3]heptane-2-carbonyl]-4-piperidyl]methoxy]nicotinonitrile;
[6-(3-cyclopropyl-1H-1,2,4-triazol-5-yl)-2-azaspiro[3.3]heptan-2-yl]-[3-[3-(2-methylsulfonylphenyl)-1-bicyclo[1.1.1]pentanyl]azetidin-1-yl]methanone;
4-[4-[1-[6-(5-cyclopropyl-4H-1,2,4-triazol-3-yl)-2-azaspiro[3.3]heptane-2-carbonyl]azetidin-3-yl]phenyl]-1H-triazole-5-carbonitrile;
4-[[2-[6-(3-cyclopropyl-1H-1,2,4-triazol-5-yl)-2-azaspiro[3.3]heptane-2-carbonyl]-2-azaspiro[3.3]heptan-6-yl]methyl]-2-(trifluoromethoxy)benzoic acid methyl ester;
5-[[2-[6-(3-cyclopropyl-1H-1,2,4-triazol-5-yl)-2-azaspiro[3.3]heptane-2-carbonyl]-2-azaspiro[3.3]heptan-6-yl]methyl]-2-(trifluoromethoxy)benzoic acid methyl ester;
4-[[2-[6-(3-cyclopropyl-1H-1,2,4-triazol-5-yl)-2-azaspiro[3.3]heptane-2-carbonyl]-2-azaspiro[3.3]heptan-6-yl]methyl]-2-(trifluoromethoxy)benzamide;
5-[[2-[6-(3-cyclopropyl-1H-1,2,4-triazol-5-yl)-2-azaspiro[3.3]heptane-2-carbonyl]-2-azaspiro[3.3]heptan-6-yl]methyl]-2-(trifluoromethoxy)benzamide;
[6-(3-cyclopropyl-1H-1,2,4-triazol-5-yl)-2-azaspiro[3.3]heptan-2-yl]-[4-[[2-mesyl-4-(trifluoromethyl)phenoxy]-methyl]piperidino]methanone;
[4-[[4-fluoro-3-(trifluoromethyl)phenoxy]methyl]piperidino]-[6-[3-(1-hydroxycyclopropyl)-1H-1,2,4-triazol-5-yl]-2-azaspiro[3.3]heptan-2-yl]methanone;
[6-(5-cyclopropyl-4H-1,2,4-triazol-3-yl)-2-azaspiro[3.3]heptan-2-yl]-[4-[[2-mesyl-4-(trifluoromethoxy)phenoxy]methyl]piperidino]methanone;
[4-(2-chloro-4-mesyl-benzyl)oxypiperidino]-[6-[3-(1-hydroxycyclopropyl)-1H-1,2,4-triazol-5-yl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[4-[[2-mesyl-4-(trifluoromethyl)phenoxy]methyl]-piperidino]methanone;
[6-[3-(1-hydroxycyclopropyl)-1H-1,2,4-triazol-5-yl]-2-azaspiro[3.3]heptan-2-yl]-[6-(4-triflylbenzyl)-2,6-diazaspiro[3.3]heptan-2-yl]methanone;
[4-[[3-fluoro-5-(trifluoromethyl)phenoxy]methyl]piperidino]-[6-[3-(1-hydroxycyclopropyl)-1H-1,2,4-triazol-5-yl]-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[4-[[4-mesyl-3-(trifluoromethyl)phenoxy]-methyl]piperidino]methanone;
[6-[3-(1-hydroxycyclopropyl)-1H-1,2,4-triazol-5-yl]-2-azaspiro[3.3]heptan-2-yl]-[4-[[2-mesyl-4-(trifluoromethoxy)phenoxy]methyl]-piperidino]methanone;
[6-(5-cyclopropyl-1H-1,2,4-triazol-3-yl)-2-azaspiro[3.3]heptan-2-yl]-[4-[[4-mesyl-3-(trifluoromethyl)phenoxy]-methyl]piperidino]methanone;
[4-(2-chloro-4-mesyl-benzyl)oxypiperidino]-[6-(5-cyclopropyl-1H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[6-(4-dimethylphosphorylbenzyl)-2-azaspiro[3.3]heptan-2-yl]methanone;
[6-[3-(1-hydroxycyclopropyl)-1H-1,2,4-triazol-5-yl]-2-azaspiro[3.3]heptan-2-yl]-[6-[[5-(trifluoromethyl)-2-pyridyl]methyl]-2-azaspiro[3.4]octan-2-yl]methanone;
[3-[3-[(5-cyclopropyl-1,3,4-oxadiazol-2-yl)methyl]-1-bicyclo[1.1.1]pentanyl]azetidin-1-yl]-[6-(3-cyclopropyl-1H-1,2,4-triazol-5-yl)-2-azaspiro[3.3]heptan-2-yl]methanone;
[6-[(3,5-difluoro-2-pyridyl)methyl]-2-azaspiro[3.4]octan-2-yl]-[6-[3-(1-hydroxycyclopropyl)-1H-1,2,4-triazol-5-yl]-2-azaspiro[3.3]heptan-2-yl]methanone;
[6-(5-cyclopropyl-4H-1,2,4-triazol-3-yl)-2-azaspiro[3.3]heptan-2-yl]-[3-[4-[3-(trifluoromethyl)-1,2,4-triazol-1-yl]phenyl]azetidin-1-yl]methanone;
[6-(5-cyclopropyl-4H-1,2,4-triazol-3-yl)-2-azaspiro[3.3]heptan-2-yl]-[6-(4-fluorophenyl)-2-azaspiro[3.3]heptan-2-yl]methanone;
[2-(4-chloro-2-fluoro-phenyl)-6-azaspiro[3.4]octan-6-yl]-[6-(5-cyclopropyl-4H-1,2,4-triazol-3-yl)-2-azaspiro[3.3]heptan-2-yl]methanone;
[2-(4-chloro-2-fluoro-phenyl)-6-azaspiro[3.4]octan-6-yl]-[6-(5-cyclopropyl-4H-1,2,4-triazol-3-yl)-2-azaspiro[3.3]heptan-2-yl]methanone;
[6-(5-cyclopropyl-4H-1,2,4-triazol-3-yl)-2-azaspiro[3.3]heptan-2-yl]-[3-[4-(3-cyclopropyl-1,2,4-triazol-1-yl)phenyl]azetidin-1-yl]methanone;
[6-(5-cyclopropyl-4H-1,2,4-triazol-3-yl)-2-azaspiro[3.3]heptan-2-yl]-[6-[(3-dimethylphosphorylphenyl)methyl]-2-azaspiro[3.3]heptan-2-yl]methanone;
[6-(5-cyclopropyl-4H-1,2,4-triazol-3-yl)-2-azaspiro[3.3]heptan-2-yl]-[6-(5-fluoro-3-pyridyl)-2,6-diazaspiro[3.3]heptan-2-yl]methanone;
[3-[6-(2-azaspiro[3.4]octan-2-yl)-3-pyridyl]azetidin-1-yl]-[6-[3-(1-hydroxycyclopropyl)-1H-1,2,4-triazol-5-yl]-2-azaspiro[3.3]heptan-2-yl]methanone;
[6-(5-cyclopropyl-4H-1,2,4-triazol-3-yl)-2-azaspiro[3.3]heptan-2-yl]-[6-[2-fluoro-4-(trifluoromethyl)phenyl]-2-azaspiro[3.3]heptan-2-yl]methanone;
[6-(5-cyclopropyl-4H-1,2,4-triazol-3-yl)-2-azaspiro[3.3]heptan-2-yl]-[6-(4-mesylphenyl)-2-azaspiro[3.3]heptan-2-yl]methanone;
[6-(5-cyclopropyl-4H-1,2,4-triazol-3-yl)-2-azaspiro[3.3]heptan-2-yl]-[6-[4-(trifluoromethyl)phenyl]-2-azaspiro[3.3]heptan-2-yl]methanone;
[6-(3-cyclopropyl-1H-1,2,4-triazol-5-yl)-2-azaspiro[3.3]heptan-2-yl]-[3-[3-[[5-(trifluoromethyl)-2-pyridyl]oxymethyl]-1-bicyclo[1.1.1]pentanyl]azetidin-1-yl]methanone;
[6-(3-cyclopropyl-1H-1,2,4-triazol-5-yl)-2-azaspiro[3.3]heptan-2-yl]-[3-[3-[[4-(trifluoromethyl)-2-pyridyl]oxymethyl]-1-bicyclo[1.1.1]pentanyl]azetidin-1-yl]methanone;
[6-(3-cyclopropyl-1H-1,2,4-triazol-5-yl)-2-azaspiro[3.3]heptan-2-yl]-[6-[[3-methoxy-5-(trifluoromethyl)pyrazin-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[difluoro-[5-(trifluoromethyl)pyrazin-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-[1-(trifluoromethyl)cyclopropyl]-1H-pyrazol-3-yl]methyl]-2-azaspiro[3.3]heptan-2-yl]methanone;
1-[[3-[1-[6-(3-cyclopropyl-1H-1,2,4-triazol-5-yl)-2-azaspiro[3.3]heptane-2-carbonyl]azetidin-3-yl]-1-bicyclo[1.1.1]pentanyl]methyl]-5-(trifluoromethyl)-2-pyridone;
1-[[3-[1-[6-(3-cyclopropyl-1H-1,2,4-triazol-5-yl)-2-azaspiro[3.3]heptane-2-carbonyl]azetidin-3-yl]-1-bicyclo[1.1.1]pentanyl]methyl]-4-(trifluoromethyl)-2-pyridone;
[6-[3-(1-aminocyclopropyl)-1H-1,2,4-triazol-5-yl]-2-azaspiro[3.3]heptan-2-yl]-[6-[[5-(trifluoromethyl)-2-pyridyl]methyl]-2-azaspiro[3.3]heptan-2-yl]methanone;
[6-(3-cyclopropyl-1H-1,2,4-triazol-5-yl)-2-azaspiro[3.3]heptan-2-yl]-[3-[3-[[4-(trifluoromethyl)pyrazol-1-yl]methyl]-1-bicyclo[1.1.1]pentanyl]azetidin-1-yl]methanone;
[6-(3-cyclopropyl-1H-1,2,4-triazol-5-yl)-2-azaspiro[3.3]heptan-2-yl]-[6-[[4-(trifluoromethylsulfonimidoyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[(4-triflylpyrazol-1-yl)methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[4-(methylsulfonimidoyl)-3-(trifluoromethyl)benzyl]-2-azaspiro[3.3]heptan-2-yl]methanone;
[6-(3-cyclopropyl-1H-1,2,4-triazol-5-yl)-2-azaspiro[3.3]heptan-2-yl]-[6-[(4-mesylpyrazol-1-yl)methyl]-2-azaspiro[3.3]heptan-2-yl]methanone;
5-[[2-[6-(3-cyclopropyl-1H-1,2,4-triazol-5-yl)-2-azaspiro[3.3]heptane-2-carbonyl]-2-azaspiro[3.3]heptan-6-yl]methyl]-3-(difluoromethyl)-2-pyridone;
[6-(3-cyclopropyl-1H-1,2,4-triazol-5-yl)-2-azaspiro[3.3]heptan-2-yl]-[7-[[3-(trifluoromethyl)isoxazol-5-yl]methyl]-2,7-diazaspiro[3.5]nonan-2-yl]methanone;
3-[[2-[6-(3-cyclopropyl-1H-1,2,4-triazol-5-yl)-2-azaspiro[3.3]heptane-2-carbonyl]-2-azaspiro[3.3]heptan-6-yl]methyl]-5-(difluoromethyl)-2-pyridone;
[6-(3-cyclopropyl-1H-1,2,4-triazol-5-yl)-2-azaspiro[3.3]heptan-2-yl]-[6-[[5-(trifluoromethyl)-1,2,4-oxadiazol-3-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[4-methyl-5-(trifluoromethyl)-1,2,4-triazol-3-yl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[3-[1-(trifluoromethyl)cyclopropyl]-1,2,4-oxadiazol-5-yl]methyl]-2,6-diazaspiro[3.3]heptan-2-yl]methanone;
1-[[2-[6-(3-cyclopropyl-1H-1,2,4-triazol-5-yl)-2-azaspiro[3.3]heptane-2-carbonyl]-2-azaspiro[3.3]heptan-6-yl]methyl]-5-(trifluoromethyl)pyrazin-2-one;
[6-(3-cyclopropyl-1H-1,2,4-triazol-5-yl)-2-azaspiro[3.3]heptan-2-yl]-[6-[[2-(2,2,2-trifluoroethyl)triazol-4-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-(2,2,2-trifluoroethyl)-1,2,4-triazol-3-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[6-(2,2,2-trifluoroethoxy)-3-pyridyl]methyl]-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[5-[1-(trifluoromethyl)cyclopropyl]imidazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[6-isopropoxy-5-(trifluoromethyl)-2-pyridyl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[(1 S)-1-[4-methyl-5-(trifluoromethyl)-2-pyridyl]ethyl]-2-azaspiro[3.3]heptan-2-yl]methanone;
[6-(3-cyclopropyl-1 #H-1,2,4-triazol-5-yl)-2-azaspiro[3.3]heptan-2-yl]-[6-[(1 #R)-1-[4-methyl-5-(trifluoromethyl)pyridin-2-yl]ethyl]-2-azaspiro[3.3]heptan-2-yl]methanone;
[6-(3-cyclopropyl-1H-1,2,4-triazol-5-yl)-2-azaspiro[3.3]heptan-2-yl]-[3-[4-(1-triflylcyclopropyl)phenyl]azetidin-1-yl]methanone;
[6-(3-cyclopropyl-1H-1,2,4-triazol-5-yl)-2-azaspiro[3.3]heptan-2-yl]-[6-[[4-(trifluoromethyl)-2-pyridyl]oxy]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethyl)-2-pyridyl]oxymethyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-(2,2,2-trifluoroethyl)triazol-4-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-(3,3,3-trifluoropropyl)pyrazol-3-yl]methyl]-2,6-diazaspiro[3.3]heptan-2-yl]methanone;
[6-[(5-cyclopropylpyrazol-1-yl)methyl]-2-azaspiro[3.3]heptan-2-yl]-[6-(3-cyclopropyl-1H-1,2,4-triazol-5-yl)-2-azaspiro[3.3]heptan-2-yl]methanone;
[3-[3-[difluoro-[4-(trifluoromethyl)phenyl]methyl]-1-bicyclo[1.1.1]pentanyl]azetidin-1-yl]-[6-[3-(1-hydroxycyclopropyl)-1H-1,2,4-triazol-5-yl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[3-[3-(3-methyl-1H-pyrazol-5-yl)-1-bicyclo[1.1.1]pentanyl]azetidin-1-yl]methanone;
[6-[3-(3,3-difluorocyclobutyl)-1H-1,2,4-triazol-5-yl]-2-azaspiro[3.3]heptan-2-yl]-[6-[4-(methylsulfonimidoyl)benzyl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[6-[(5-mesyl-2-pyridyl)methyl]-2-azaspiro[3.3]heptan-2-yl]methanone;
[6-[3-(3,3-difluorocyclobutyl)-1H-1,2,4-triazol-5-yl]-2-azaspiro[3.3]heptan-2-yl]-[3-[2-[3-(trifluoromethyl)azetidin-1-yl]pyrimidin-5-yl]azetidin-1-yl]methanone;
[4-[6-(5-cyclopropyl-4H-1,2,4-triazol-3-yl)-2-azaspiro[3.3]heptane-2-carbonyl]piperazino]-(3-fluorophenyl)methanone;
4-[[1-[6-(3-cyclopropyl-1H-1,2,4-triazol-5-yl)-2-azaspiro[3.3]heptane-2-carbonyl]-4-piperidyl]oxymethyl]-2-methoxy-benzamide;
[3-[4-(2-chloro-4-mesyl-phenyl)phenyl]azetidin-1-yl]-[6-(1H-1,2,4-triazol-5-yl)-2-azaspiro[3.3]heptan-2-yl]methanone;
[6-[2-fluoro-4-(trifluoromethyl)phenyl]-2-azaspiro[3.3]heptan-2-yl]-[6-(1H-1,2,4-triazol-5-yl)-2-azaspiro[3.3]heptan-2-yl]methanone;
[6-(1H-1,2,4-triazol-5-yl)-2-azaspiro[3.3]heptan-2-yl]-[6-[[[1-(trifluoromethyl)cyclopropyl]amino]methyl]-2-azaspiro[3.3]heptan-2-yl]methanone;
[6-[4-mesyl-3-(trifluoromethyl)benzyl]-2-azaspiro[3.3]heptan-2-yl]-[6-(1H-1,2,4-triazol-5-yl)-2-azaspiro[3.3]heptan-2-yl]methanone;
[6-(1H-1,2,4-triazol-5-yl)-2-azaspiro[3.3]heptan-2-yl]-[6-[[4-(trifluoromethyl)-2-pyridyl]methyl]-2-azaspiro[3.3]heptan-2-yl]methanone;
[6-(1H-1,2,4-triazol-5-yl)-2-azaspiro[3.3]heptan-2-yl]-[6-[[6-(trifluoromethyl)-3-pyridyl]methyl]-2-azaspiro[3.3]heptan-2-yl]methanone;
[6-(1H-1,2,4-triazol-5-yl)-2-azaspiro[3.3]heptan-2-yl]-[6-[[5-(trifluoromethyl)-2-pyridyl]methyl]-2-azaspiro[3.3]heptan-2-yl]methanone;
[6-[(3,5-difluoro-2-pyridyl)methyl]-2-azaspiro[3.3]heptan-2-yl]-[6-(1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)-1H-indazol-6-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[2-(trifluoromethyl)imidazo[1,2-a]pyridin-6-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[2-(trifluoromethyl)imidazo[1,2-a]pyrazin-6-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)imidazo[1,5-a]pyridin-6-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[2-(trifluoromethyl)-[1,2,4]triazolo[1,5-a]pyridin-6-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)-1H-indazol-5-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)-[1,2,4]triazolo[4,3-a]pyridin-6-yl]methyl]-2-azaspiro[3.3]heptan-2-yl]methanone; and
[6-(3-cyclopropyl-1H-1,2,4-triazol-5-yl)-2-azaspiro[3.3]heptan-2-yl]-[6-[[2-(trifluoromethyl)pyrazolo[1,5-a]pyrimidin-6-yl]methyl]-2-azaspiro[3.3]heptan-2-yl]methanone.

14. The compound of formula (I) according to claim 13, or a pharmaceutically acceptable salt thereof, selected from:
[6-(5-cyclopropyl-4H-1,2,4-triazol-3-yl)-2-azaspiro[3.3]heptan-2-yl]-[6-[[5-(trifluoromethyl)pyrazin-2-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[5-(trifluoromethyl)pyrimidin-2-yl]methyl]-2-azaspiro[3.3]heptan-2-yl]methanone;
[[6-(5-cyclopropyl-4H-1,2,4-triazol-3-yl)-2-azaspiro[3.3]heptan-2-yl]-[6-[[6-(trifluoromethyl)pyrimidin-4-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-(trifluoromethyl)pyrazol-4-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[4-(trifluoromethyl)pyrimidin-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)-1H-pyrazol-5-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[1-cyclopropyl-3-(trifluoromethyl)pyrazol-4-yl]methyl]-2-azaspiro[3.3]heptan-2-yl]methanone;
1-[[2-[6-(3-cyclopropyl-1H-1,2,4-triazol-5-yl)-2-azaspiro[3.3]heptane-2-carbonyl]-2-azaspiro[3.3]heptan-6-yl]methyl]-5-(trifluoromethyl)-2-pyridone;
[6-(3-cyclopropyl-1H-1,2,4-triazol-5-yl)-2-azaspiro[3.3]heptan-2-yl]-[6-[[5-(trifluoromethyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[4-(trifluoromethyl)triazol-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-(4-triflylbenzyl)-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[5-(trifluoromethyl)pyrazin-2-yl]methyl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-(4-triflylbenzyl)-2,6-diazaspiro[3.3]heptan-2-yl]methanone;
[6-(3-cyclopropyl-1H-1,2,4-triazol-5-yl)-2-azaspiro[3.3]heptan-2-yl]-[6-[[3-methyl-5-(trifluoromethyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-methyl-3-(trifluoromethyl)pyrazol-1-yl]methyl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[6-(trifluoromethyl)pyridazin-3-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[7-[[2-methyl-4-(trifluoromethyl)pyrazol-3-yl]methyl]-2,7-diazaspiro[3.5]nonan-2-yl]methanone;
[6-[3-(3,3-difluorocyclobutyl)-1H-1,2,4-triazol-5-yl]-2-azaspiro[3.3]heptan-2-yl]-[6-[[5-(trifluoromethyl)pyrazin-2-yl]methyl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[3-(2-fluoro-4-triflyl-benzyl)oxyazetidin-1-yl]methanone;
[6-[3-(1-hydroxycyclopropyl)-1H-1,2,4-triazol-5-yl]-2-azaspiro[3.3]heptan-2-yl]-[6-[[1-methyl-5-(trifluoromethyl)pyrazol-3-yl]methyl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[3-[5-[[1-(trifluoromethyl);
5-[[2-[6-[3-(1-fluorocyclopropyl)-1H-1,2,4-triazol-5-yl]-2-azaspiro[3.3]heptane-2-carbonyl]-2-azaspiro[3.3]heptan-6-yl]methyl]-3-(trifluoromethyl)picolinonitrile;
[6-[3-(1-fluorocyclopropyl)-1H-1,2,4-triazol-5-yl]-2-azaspiro[3.3]heptan-2-yl]-[6-[[5-(trifluoromethyl)-2-pyridyl]methyl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[4-(trifluoromethyl)pyrazol-1-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[3-[4-[3-(difluoromethyl)-5-methyl-1,2,4-triazol-1-yl]phenyl]azetidin-1-yl]methanone;
[6-(3-cyclopropyl-1H-1,2,4-triazol-5-yl)-2-azaspiro[3.3]heptan-2-yl]-[7-[[5-(trifluoromethyl)isoxazol-3-yl]methyl]-2,7-diazaspiro[3.5]nonan-2-yl]methanone;
[6-(3-cyclopropyl-1H-1,2,4-triazol-5-yl)-2-azaspiro[3.3]heptan-2-yl]-[7-(3-triflylbenzyl)-2,7-diazaspiro[3.4]octan-2-yl]methanone;
[6-[3-(1-fluorocyclopropyl)-1H-1,2,4-triazol-5-yl]-2-azaspiro[3.3]heptan-2-yl]-[3-[4-(trifluoromethylsulfonimidoyl)phenyl]azetidin-1-yl]methanone; methanone;
[6-[3-(1-fluorocyclopropyl)-1H-1,2,4-triazol-5-yl]-2-azaspiro[3.3]heptan-2-yl]-[6-[[2-(trifluoromethyl)pyrimidin-5-yl]methyl]-2-azaspiro[3.3]heptan-2-yl]methanone;
4-[[2-[6-[3-(1-fluorocyclopropyl)-1H-1,2,4-triazol-5-yl]-2-azaspiro[3.3]heptane-2-carbonyl]-2-azaspiro[3.3]heptan-6-yl]methyl]-2-(trifluoromethyl)benzonitrile;
[6-(3-cyclopropyl-1H-1,2,4-triazol-5-yl)-2-azaspiro[3.3]heptan-2-yl]-[3-[3-[5-(2,2-dimethylpropyl)-1,3,4-thiadiazol-2-yl]-1-bicyclo[1.1.1]-pentanyl]azetidin-1-yl]methanone;
5-[[2-[6-[3-(1-fluorocyclopropyl)-1H-1,2,4-triazol-5-yl]-2-azaspiro[3.3]heptane-2-carbonyl]-2-azaspiro[3.3]heptan-6-yl]methyl]-2-(trifluoromethyl)benzonitrile;
[6-[3-(1-fluorocyclopropyl)-1H-1,2,4-triazol-5-yl]-2-azaspiro[3.3]heptan-2-yl]-[6-[[1-methyl-5-(trifluoromethyl)pyrazol-3-yl]methyl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-(3-triflylbenzyl)-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[5-(trifluoromethyl)pyrimidin-2-yl]methyl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-[(5-triflyl-2-pyridyl)methyl]-2-azaspiro[3.3]heptan-2-yl]methanone;
[6-(3-cyclopropyl-1H-1,2,4-triazol-5-yl)-2-azaspiro[3.3]heptan-2-yl]-[6-(3-fluoro-5-triflyl-benzyl)-2-azaspiro[3.3]heptan-2-yl]methanone;
[6-(3-cyclopropyl-1H-1,2,4-triazol-5-yl)-2-azaspiro[3.3]heptan-2-yl]-[6-[[3-(trifluoromethyl)-1,2,4-oxadiazol-5-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[3-[3-[[5-(trifluoromethyl)pyrazin-2-yl]-amino]-1-bicyclo[1.1.1]-pentanyl]azetidin-1-yl]methanone;
[6-(3-cyclopropyl-1H-1,2,4-triazol-5-yl)-2-azaspiro[3.3]heptan-2-yl]-[6-[[4-[1-(trifluoromethyl)cyclopropyl]-pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[2-methyl-5-(trifluoromethyl)triazol-4-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[(5-triflyl-3-pyridyl)methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-(trifluoromethylsulfonimidoyl)-2-pyridyl]methyl]-2-azaspiro[3.3]heptan-2-yl]methanone;
[6-[[5-(trifluoromethyl)pyrazin-2-yl]methyl]-2-azaspiro[3.3]heptan-2-yl]-[6-[3-(trifluoromethyl)-1H-1,2,4-triazol-5-yl]-2-azaspiro[3.3]heptan-2-yl]methanone;
[6-[3-(1-fluorocyclopropyl)-1H-1,2,4-triazol-5-yl]-2-azaspiro[3.3]heptan-2-yl]-[6-[[5-[1-(trifluoromethyl);
[6-(3-cyclopropyl-1H-1,2,4-triazol-5-yl)-2-azaspiro[3.3]heptan-2-yl]-[6-[(5-triflyl-2-pyridyl)methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[difluoro-[6-(trifluoromethyl)-3-pyridyl]methyl]-2-azaspiro[3.3]heptan-2-yl]methanone;
5-[[2-[6-(5-cyclopropyl-4H-1,2,4-triazol-3-yl)-2-azaspiro[3.3]heptane-2-carbonyl]-2-azaspiro[3.3]heptan-6-yl]methyl]-2-(trifluoromethyl)nicotinonitrile;
5-[[2-[6-(5-cyclopropyl-4H-1,2,4-triazol-3-yl)-2-azaspiro[3.3]heptane-2-carbonyl]-2-azaspiro[3.3]heptan-6-yl]methyl]-2-(trifluoromethyl)isonicotinonitrile;
[6-(3-cyclopropyl-1H-1,2,4-triazol-5-yl)-2-azaspiro[3.3]heptan-2-yl]-[6-[[3-(trifluoromethyl)pyrazol-1-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[fluoro-[6-(trifluoromethyl)pyridin-3-yl]methyl]-2-azaspiro[3.3]heptan-2-yl]methanone;
[6-[(5-cyclopropyl-3-methyl-pyrazol-1-yl)methyl]-2-azaspiro[3.3]heptan-2-yl]-[6-(3-cyclopropyl-1H-1,2,4-triazol-5-yl)-2-azaspiro[3.3]heptan-2-yl]methanone;
[6-[(3-cyclopropyl-5-methyl-pyrazol-1-yl)methyl]-2-azaspiro[3.3]heptan-2-yl]-[6-(3-cyclopropyl-1H-1,2,4-triazol-5-yl)-2-azaspiro[3.3]heptan-2-yl]methanone;
[6-(3-cyclopropyl-1H-1,2,4-triazol-5-yl)-2-azaspiro[3.3]heptan-2-yl]-[6-[2-fluoro-4-(trifluoromethylsulfonimidoyl)benzyl]-2-azaspiro[3.3]heptan-2-yl]methanone;
[6-[3-(trifluoromethyl)-1H-1,2,4-triazol-5-yl]-2-azaspiro[3.3]heptan-2-yl]-[6-(4-triflylbenzyl)-2,6-diazaspiro[3.3]heptan-2-yl]methanone;
[6-(5-cyclopropyl-4H-1,2,4-triazol-3-yl)-2-azaspiro[3.3]heptan-2-yl]-[6-[[4-(trifluoromethyl)pyrazol-1-yl]methyl]-2-azaspiro[3.3]heptan-2-yl]methanone;
[6-(5-cyclopropyl-4H-1,2,4-triazol-3-yl)-2-azaspiro[3.3]heptan-2-yl]-[6-[[3-(trifluoromethyl)-1,2,4-thiadiazol-5-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[difluoro-[5-(trifluoromethyl)pyrazin-2-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[5-[1-(trifluoromethyl)cyclopropyl]-1H-pyrazol-3-yl]methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[(4-triflylpyrazol-1-yl)methyl]-2-azaspiro[3.3]heptan-2-yl]methanone;
[6-(3-cyclopropyl-1H-1,2,4-triazol-5-yl)-2-azaspiro[3.3]heptan-2-yl]-[6-[[3-[1-(trifluoromethyl)cyclopropyl]-1,2,4-oxadiazol-5-yl]methyl]-2,6-diazaspiro[3.3]heptan-2-yl]methanone;
1-[[2-[6-(3-cyclopropyl-1H-1,2,4-triazol-5-yl)-2-azaspiro[3.3]heptane-2-carbonyl]-2-azaspiro[3.3]heptan-6-yl]methyl]-5-(trifluoromethyl)pyrazin-2-one; and
[6-(3-cyclopropyl-1H-1,2,4-triazol-5-yl)-2-azaspiro[3.3]heptan-2-yl]-[6-[[1-(2,2,2-trifluoroethyl)-1,2,4-triazol-3-yl]methyl]-2-azaspiro[3.3]heptan-2-yl]methanone.

15. A process for manufacturing a compound of formula (I) according to any one of claims 1 to 14, or a pharmaceutically acceptable salt thereof, comprising reacting a compound of formula 2; 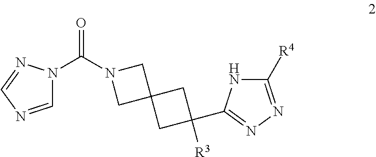
wherein R³ and R⁴ are as defined in any one of claims 1 to 14, with a compound of formula 1; 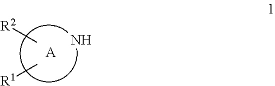
wherein R³ and R⁴ are as defined in any one of claims 1 to 14; by heating in a solvent, such as DMF or CH₃CN, in the presence of a base, such as DIPEA, to form said compound of formula (I).

16. The compound of formula (I) according to any one of claims 1 to 14, or a pharmaceutically acceptable salt thereof, when manufactured according to the process of claim 15.

17. A compound of formula (I) according to any one of claims 1 to 14, or a pharmaceutically acceptable salt thereof, for use as therapeutically active substance.

18. A pharmaceutical composition comprising a compound of formula (I) according to any one of claims 1 to 14, or a pharmaceutically acceptable salt thereof, and a therapeutically inert carrier.

19. A method for the treatment or prophylaxis of neuroinflammation, neurodegenerative diseases, pain, cancer, mental disorders and/or inflammatory bowel disease in a mammal, the method comprising administering a therapeutically effective amount of a compound of formula (I) according to any one of claims 1 to 14, or of a pharmaceutically acceptable salt thereof, or of a pharmaceutical composition according to claim 18, to said mammal.

20. The method according to claim 19, wherein said neuroinflammation, neurodegenerative diseases, pain, cancer and mental disorders are selected from multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain.

21. A compound according to any one of claims 1 to 14, or a pharmaceutically acceptable salt thereof, or a pharmaceutical composition according to claim 18, for use in a method according to claim 19 or 20.

22. Use of a compound according to any one of claims 1 to 14, or of a pharmaceutically acceptable salt thereof, or of a pharmaceutical composition according to claim 18, in a method according to claim 20 or 21.

23. Use of a compound according to any one of claims 1 to 14, or of a pharmaceutically acceptable salt thereof, in the preparation of a medicament for use in a method according to claim 20 or 21.

24. The invention as described hereinbefore.
